(19) United States
(12) Patent Application Publication
(10) Pub. No.: US 2025/0353864 A1
(43) Pub. Date: Nov. 20, 2025

(54) INDAZOLE CONTAINING MACROCYCLES AND THEIR USE

(71) Applicant: BLOSSOMHILL THERAPEUTICS, INC., San Diego, CA (US)

(72) Inventors: Jingrong Jean CUI, San Diego, CA (US); Eugene Yuanjin RUI, San Diego, CA (US); Evan W. ROGERS, San Diego, CA (US); Anindya SARKAR, San Diego, CA (US); Jane UNG, San Diego, CA (US)

(21) Appl. No.: 18/871,723

(22) PCT Filed: Jun. 7, 2023
(86) PCT No.: PCT/US2023/068066

§ 371 Date: Dec. 4, 2024

Related U.S. Application Data
(60) Provisional application No. 63/350,309, filed on Jun. 8, 2022.
(60) Provisional application No. 63/350,310, filed on Jun. 8, 2022.
(60) Provisional application No. 63/503,879, filed on May 23, 2023.

(51) Int. Cl. C07D 498/22; A61K 31/4162; A61K 31/424; A61K 31/429; A61K 31/437; A61K 31/439; A61K 31/454; A61K 31/496; A61K 31/4985; A61K 31/5377; C07D 491/18; C07D 491/22; C07D 513/22

(52) U.S. Cl. CPC C07D 498/22; A61K 31/4162; A61K 31/424; A61K 31/429; A61K 31/437; A61K 31/439; A61K 31/454; A61K 31/496; A61K 31/4985; A61K 31/5377; C07D 491/18; C07D 491/22; C07D 513/22

(57) ABSTRACT

The present disclosure relates to indazole containing macrocyclic compounds, pharmaceutical compositions containing macrocyclic compounds, and methods of using macrocyclic compounds to treat disease, such as cancer.

### RELATED APPLICATIONS

This application claims the benefit of U.S. Provisional Application No. 63/350,309, filed Jun. 8, 2022, U.S. Provisional Application No. 63/350,310, filed Jun. 8, 2022, and U.S. Provisional Application No. 63/503,879, filed May 23, 2023, the entire disclosures of all of which are incorporated herein by reference.

### TECHNICAL FIELD

The present disclosure relates to indazole containing macrocyclic compounds, pharmaceutical compositions containing macrocyclic compounds, and methods of using macrocyclic compounds to treat disease, such as cancer.

### BACKGROUND

Protein kinases are tightly regulated signaling proteins that orchestrate the activation of signaling cascades by phosphorylating target proteins in response to extracellular and intracellular stimuli. The human genome encodes approximately 518 protein kinases (Manning G, et al The protein kinase complement of the human genome. Science. 2002, 298:1912-34). Dysregulation of kinase activity is associated with many diseases, including cancers, and cardiovascular, degenerative, immunological, infectious, inflammatory, and metabolic diseases (Levitzki, A. Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et al New Perspectives, Opportunities, and Challenges in Exploring the Human Protein Kinome. Cancer Res. 2018, 78:15-29). The critical role of kinases in cancer and other diseases makes them attractive targets for drug inventions with 62 small molecule kinase inhibitors have been approved and 55 of them for cancer targeted therapies (Roskoski R Jr, Properties of FDA-approved Small Molecule Protein Kinase Inhibitors: A 2021 Update. Pharmacol Res 2021, 165:105463). Although kinase inhibitors have achieved dramatic success in cancer targeted therapies, the development of treatment resistance has remained as a challenge for small molecule kinase inhibitors. Acquired secondary mutations within kinase domain during the treatment often lead to treatment resistance to kinase inhibitors (Pottier C, et al Tyrosine Kinase Inhibitors in Cancer: Breakthrough and Challenges of Targeted Therapy. Cancers (Basel), 2020, 12:731). Resistance can also arise from subpopulations of tolerant/persister cells that survive in the presence of the treatment. Different processes contribute to the emergence of tolerant persister cells, including pathway rebound through the release of negative feedback loops, transcriptional rewiring mediated by chromatin remodeling and autocrine/paracrine communication among tumor cells and within the tumor microenvironment (Swayden M, et al Tolerant/Persister Cancer Cells and the Path to Resistance to Targeted Therapy. Cells 2020, 9, 2601). Therefore, it is necessary to invent kinase inhibitors that can target not only the kinase oncogenic drivers, overcome most frequent resistance mutations, but also tolerant persister cancer cells for overcoming resistance, achieving better efficacy and longer disease control.

Non-small-cell lung cancer (NSCLC) is the leading cause of cancer mortality worldwide (World Health Organisation. Cancer Fact Sheet 2017). Activating EGFR mutations have been reported in approximately 10% to 15% of cases of adenocarcinoma in white patients and 50% of cases in Asian patients (Chan B A, Hughes B G. Targeted therapy for non-small cell lung cancer: current standards and the promise of the future. Transl Lung Cancer Res 2015; 4:36-54). The two most frequent EGFR alterations found in NSCLC tumors are short in-frame deletions in exon 19 (del19) of the EGFR gene and L858R, a single missense mutation in exon 21 (Konduri K. et al. EGFR Fusions as Novel Therapeutic Targets in Lung Cancer. Cancer Discovery 2016, 6:601-11).

The first-generation reversible EGFR inhibitors, erlotinib and gefitinib are superior to chemotherapy in patients with advanced EGFR mutation-positive (Del19 or L858R) NSCLC and have been used as first-line standard of care in this setting. However, most patients will develop resistance to gefitinib or erlotinib with 50% to 70% of tumors developing EGFR T790M gatekeeper mutation with time of treatment (Sequist L V, et al. Genotypic and histological evolution of lung cancers acquiring resistance to EGFR inhibitors. Sci Transl Med 2011; 3:75ra26). The second generation of EGFR inhibitors afatinib and dacomitinib are covalent, irreversible EGFR inhibitors that also inhibit HER² and ERB4 of the ERB family (Li D, et al. BIBW2992, an irreversible EGFR/HER² inhibitor highly effective in preclinical lung cancer models. Oncogene 2008; 27: 4702-11; Ou S H, Soo R A. Dacomitinib in lung cancer: a “lost generation” EGFR tyrosine-kinase inhibitor from a bygone era?Drug Des Devel Ther 2015; 9:5641-53).

Although afatinib and dacomitinib are more potent EGFR inhibitors approved as first-line therapy for advanced EGFR mutation-positive (Del19 or L858R) NSCLC with longer progression free survival time (PFS) in comparison with gefitinib and erlotinib, EGFR T790M has been developed with time of treatment with afatinib (Tanaka K, et al. Acquisition of the T790M resistance mutation during afatinib treatment in EGFR tyrosine kinase inhibitor-naive patients with non-small cell lung cancer harboring EGFR mutations. Onco-target 2017; 8:68123-30). EGFR T790M confers resistance to dacomitinib In vitro studies (Kobayashi Y, et al. EGFR T790M and C797S mutations as mechanisms of acquired resistance to dacomitinib. J Thorac Oncol 2018; 13: 727-31). The third-generation EGFR inhibitor Osimertinib is also an irreversible inhibitor targeting both EGFR activating mutations (Del19 and L858R) and T790M resistant double mutations, with selectivity over the wild-type EGFR (Finlay M R, et al. Discovery of a potent and selective EGFR inhibitor (AZD9291) of both sensitizing and T790M resistance mutations that spares the wild type form of the receptor. J Med Chem 2014; 57:8249-67). Osimertinib was first approved for patients with metastatic EGFR T790M mutation-positive NSCLC after failure of first-line EGFR inhibitors, and later approved in the first-line setting for patients with EGFR mutation-positive NSCLC following the phase III FLAURA trial with head-to-head trials comparing with erlotinib or gefitinib (Soria J C, et al. Osimertinib in untreated EGFR-mutated advanced non-small-cell lung cancer. N Engl J Med 2018; 378:113-25). The mutation C797S at the EGFR covalent binding residue with irreversible EGFR inhibitor Osimertinib has been detected in Osimertinib-resistant patients (Ramalingam S S, et al. Mechanisms of acquired resistance to first-line osimertinib: preliminary data from the phase III FLAURA study. Presented at the ESMO 2018).

Therefore, it is necessary to develop a new generation reversible EGFR inhibitor that are potent against oncogenic driver EGFR mutations, such as L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S, as well as other emerging and established resistance mutations, while maintaining good selectivity over wild-type EGFR.

The proviral integration for the Moloney murine leukemia virus (PIM) kinases are oncogenic serine/threonine kinases that phosphorylate a wide range of substrates that regulate several of the hallmarks of cancer including tumor metabolism, survival, metastasis, immune evasion and inflammation (Toth R K, Warfel N A. Targeting PIM Kinases to Overcome Therapeutic Resistance in Cancer. Mol Cancer Ther. 2021, 20(1):3-10). PIM kinases interact with numerous major oncogenic players, including stabilization of p53, synergism with c-Myc, and notable parallel signaling with PI3K/Akt. The aberrant PIM kinase activity plays an important role in resistance mechanisms of chemotherapy, radiotherapy, anti-angiogenic therapies and targeted therapies, providing a rationale for co-targeting treatment strategies for a more durable patient response (Malone T, et al Current perspectives on targeting PIM kinases to overcome mechanisms of drug resistance and immune evasion in cancer. Pharmacol Ther 2020 March; 207).

The anaplastic lymphoma kinase (ALK) is a member of the family of insulin-like tyrosine kinase receptors involved in the oncogenesis of several tumor types. Approximately 5% of patients with non-small cell lung cancer (NSCLC) harbor rearrangement in the anaplastic lymphoma kinase (ALK) gene (Soda, M. et al. Identification of the transforming EML4-ALK fusion gene in non-small-cell lung cancer. Nature 2007, 448, 561-566). ALK inhibitors have been approved by FDA as the standard of care in the first- and second-line treatment of ALK-rearranged NSCLC patients. However, as complete response to ALK inhibitors is rare, almost all patients with ALK-rearranged NSCLC inevitably acquire resistance to ALK inhibitors, resulting in tumor recurrence. Drug resistance mechanisms include ALK-independent and ALK-dependent processes. ALK-independent resistance mechanisms involve the activation of bypass pathways, such as EGFR, c-MET, KRAS, and AXL or transformation into small cell lung cancer (Gainor, J. F. et al. Molecular mechanisms of resistance to first- and second generation ALK inhibitors in ALK-rearranged lung cancer. Cancer Discov. 2016, 6, 1118-1133). Although five ALK inhibitors have been approved, they have a limited clinical ability to overcome ALK-independent resistance mechanisms. Therefore, it is necessary to develop next generation multitargeted ALK inhibitors with ability targeting not only primary ALK fusions and ALK secondary resistance mutations, but also targeting mechanisms associated with tolerant persister cancer cells for better efficacy and longer duration of response.

The proviral integration for the Moloney murine leukemia virus (PIM) kinases are oncogenic serine/threonine kinases that phosphorylate a wide range of substrates that regulate several of the hallmarks of cancer including tumor metabolism, survival, metastasis, immune evasion and inflammation (Toth R K, Warfel N A. Targeting PIM Kinases to Overcome Therapeutic Resistance in Cancer. Mol Cancer Ther. 2021, 20(1):3-10). PIM kinases interact with numerous major oncogenic players, including stabilization of p53, synergism with c-Myc, and notable parallel signaling with PI3K/Akt. The aberrant PIM kinase activity plays an important role in resistance mechanisms of chemotherapy, radiotherapy, anti-angiogenic therapies and targeted therapies, providing a rationale for co-targeting treatment strategies for a more durable patient response (Malone T, et al Current perspectives on targeting PIM kinases to overcome mechanisms of drug resistance and immune evasion in cancer. Pharmacol Ther 2020 March; 207).

Cdc-like kinases (CLKs) are evolutionary conserved dual-specificity kinases that are able to phosphorylate serine, threonine, and tyrosine residues. CLKs catalyze the phosphorylation of SR proteins, serine, and arginine-rich splicing factors 1-12 (SRSF1-12), which regulate the spliceosome molecular machinery (Martin Moyano P, et al Cdc-Like Kinases (CLKs): Biology, Chemical Probes, and Therapeutic Potential. Int J Mol Sci 2020, 21(20):7549). Dysregulation of alternative splicing is a feature of cancer. High-frequency mutations of SF3B1 or SRSF2 have been described in patients with myelodysplastic syndromes (MDS), chronic myelomonocytic leukemia, and acute myeloid leukemia (AML) (Papaemmanuil et al, Genomic classification and prognosis in acute myeloid leukemia. N Engl J Med. 2016, 374:2209-2221). In addition, mutations in splicing-related genes have also been found in various solid cancers, including lung, breast, and pancreatic cancers (Dvinge H, et al RNA splicing factors as oncoproteins and tumour suppressors. Nat Rev Cancer 2016, 16: 413-430). The modulation of pre-mRNA splicing via inhibition of CLK kinases is an attractive anti-neoplastic strategy, especially for the cancers that exhibit aberrant pre-mRNA splicing.

Therefore, it is necessary to develop a new generation of multitargeted EGFR inhibitors and multitargeted ALK inhibitors that are potent against oncogenic driver EGFR mutations, ALK fusions, and point mutations, other emerging and established EGFR and ALK resistance mutations, as well as emerging resistance targets for tolerant/persistent cancer cells, e.g. PIM kinases and CLK kinases.

### SUMMARY

In one aspect, the disclosure provides a compound of the formula I, or a pharmaceutically acceptable salt thereof,

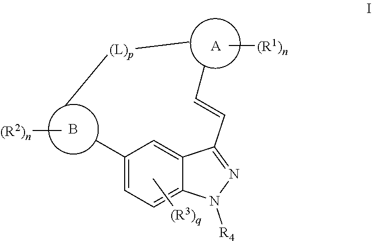

wherein R¹, R², R³, R⁴, A, B, L, m, n, p, and q are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷— fragment directly covalently to ring A.

In some embodiments, the disclosure provides a compound of the formula II, or a

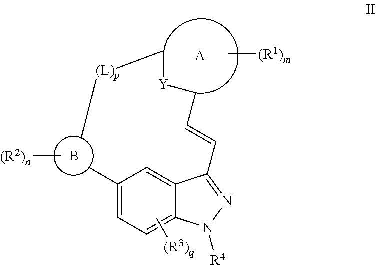

wherein R¹, R², R³, R⁴, A, B, L, Y, m, n, p, and q, are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷— fragment directly covalently to ring A.

In some embodiments, the disclosure provides a compound of the formula III, or a pharmaceutically acceptable salt thereof,

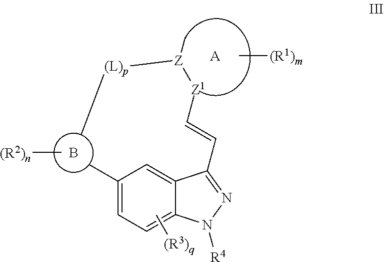

wherein R¹, R², R³, R⁴, A, B, L, Z, Z¹, X¹, X², X³, m, n, p, and q, are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷— fragment directly covalently to ring A.

In some embodiments, the disclosure provides a compound of the formula IV, or a

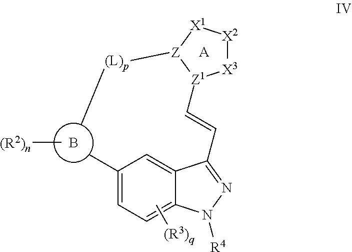

wherein R¹, R², R³, R⁴, A, B, L, Z, Z¹, X¹, X², X³, m, n, p, and q, are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷— fragment directly covalently to ring A.

In further aspects, the disclosure relates to a pharmaceutical composition comprising at least one compound of Formula (I)-(XII) or a pharmaceutically acceptable salt thereof.

Pharmaceutical compositions according to the disclosure may further comprise a pharmaceutically acceptable excipient.

In further aspects, the disclosure relates to a compound of Formula (I)-(XII), or a pharmaceutically acceptable salt thereof, for use as a medicament.

In further aspects, the disclosure relates to a method of treating disease, such as cancer comprising administering to a subject in need of such treatment an effective amount of at least one compound of Formula (I)-(XII), or a pharmaceutically acceptable salt thereof.

In further aspects, the disclosure relates to use of a compound of Formula (I)-(XII), or a pharmaceutically acceptable salt thereof, in the preparation of a medicament for the treatment of disease, such as cancer, and the use of such compounds and salts for treatment of such diseases.

In further aspects, the disclosure relates to a method of inhibiting EGFR, including the certain mutations as described herein, PIM kinases, and/or CLK kinases, comprising contacting a cell comprising one or more of an aberrant EGFR, including the certain mutations as described herein, a PMI kinase, and/or CLK kinase, with an effective amount of at least one compound of Formula (I)-(XII), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I

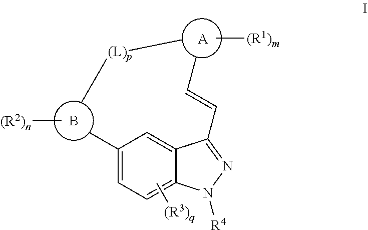

wherein
ring A is a 5- to 10-membered heteroarylene or C₆-C₁₀ arylene;
ring B is a 5-membered heteroarylene;
each L is independently —O—, —S—, —S(O)—, —S(O)₂—, —N(R⁵)C(O)—, —C(O)N(R⁵)—, —N(R⁵)—, —N(R⁵)S(O)—, —S(O)N(R⁵)—, —N(R⁵)S(O)₂—, —S(O)₂N(R⁵)—, or —C(R⁶)(R⁷)—, provided that (L)ₚ does not comprise an O—O, S—O, or N—N bond;
each R¹, R², and R³ when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NRaS(O)R^{b}, —NRaS(O)₂R^{b}, —NRaS(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, R^{c}, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
or two R¹, two R², or two R³, taken together with the atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹, R² and R³ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁴ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{e}, or —S(O)₂OR^{e};
each R⁵, when present, is independently H, deuterium, C₁-C₆ alkyl, C₂-C₆ alkenyl, C₂-C₆ alkynyl, C₃-C₆ cycloalkyl, —C(O)R^{a}, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{e}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{e}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁶ and R⁷, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)ORf, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
or two of R⁵, R⁶ and R⁷, taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁵, R⁶ and R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{c}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{e} and R^{d} or R^{c} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4;
n is 0, 1, 2, or 3;
p is 4, 5, 6, 7, 8 or 9; and
q is 0, 1, or 2;
or a pharmaceutically acceptable salt thereof;
provided that the compound is not of the formula

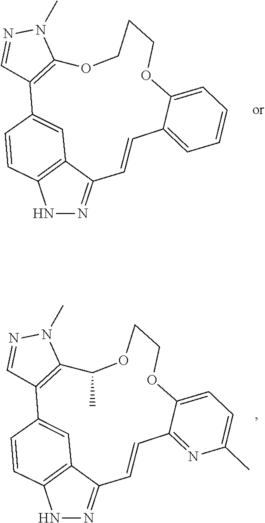 [SMILES: Cn1ncc2c1OCCCOc1ccccc1/C=C/c1n[nH]c3ccc-2cc13 ; Cc1ccc2c(n1)/C=C/c1n[nH]c3ccc(cc13)-c1cnn(C)c1[C@@H](C)OCCO2]

or a pharmaceutically acceptable salt thereof.

2. A compound of the formula 1

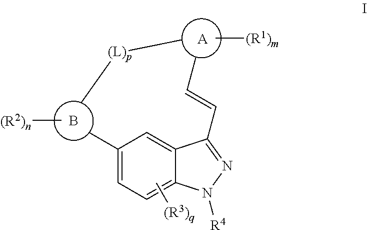

wherein
ring A is a 5- to 10-membered heteroarylene or C₆-C₁₀ arylene;
ring B is a 5-membered heteroarylene;
each L is independently —O—, —S—, —S(O)—, —S(O)₂—, —N(R⁵)C(O)—, —C(O)N(R⁵)—, —N(R⁵)—, —N(R⁵)S(O)—, —S(O)N(R⁵)—, —N(R⁵)S(O)₂—, —S(O)₂N(R⁵)—, or —C(R⁶)(R⁷)—, provided that (L)ₚ does not comprise an O—O, S—O, or N—N bond;
each R¹, R², and R³ when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
R⁴ is H, deuterium, —C(O)RC, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR^{c};
each R⁵, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁶ and R⁷, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
or two of R⁵, R⁶ and R⁷, taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁵, R⁶ and R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{c}, —S(O)₂R^{e}, —S(O)NR^{e}R^{f}, —S(O)₂NR^{e}R^{f}, —NR^{e}R^{f}, —NR^{c}C(O)R^{f}, —NR^{c}C(O)OR^{f}, —NR^{c}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₅-C₁₀ aryl, 5- to 10-membered heteroaryl, and C₁-C₆ alkylene-5- to 10-membered heteroaryl, or R^{a} and R^{b} or RC and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₅ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, 3, or 4;
n is 0, 1, 2, or 3;
p is 4, 5, 6, 7, 8 or 9; and
q is 0, 1, or 2;
or a pharmaceutically acceptable salt thereof;
provided that the compound is not of the formula

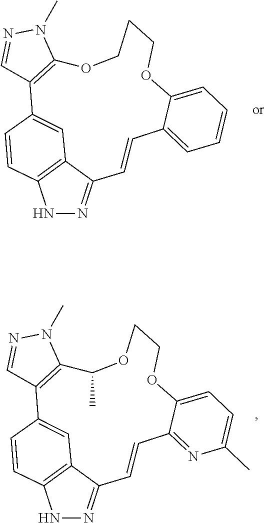 [SMILES: Cn1ncc2c1OCCCOc1ccccc1/C=C/c1n[nH]c3ccc-2cc13 ; Cc1ccc2c(n1)/C=C/c1n[nH]c3ccc(cc13)-c1cnn(C)c1[C@@H](C)OCCO2]

or a pharmaceutically acceptable salt thereof.

3. The compound of clause 1 or 2, or a pharmaceutically acceptable salt thereof, wherein (L)ₚ does not comprise a —NR⁵C(O)— directly covalently attached to ring A.

4. The compound of any one of the preceding clauses, or a pharmaceutically acceptable salt thereof, wherein ring A is a C₆-C₁₀ arylene, and m is 0, 1, or 2.

5. The compound of any one of the preceding clauses, or a pharmaceutically acceptable salt thereof, wherein ring A is a phenylene, and m is 0, 1, or 2.

6. The compound of any one of the preceding clauses, or a pharmaceutically acceptable salt thereof, wherein ring A is a phenylene, and m is 0 or 1.

7. The compound of any one of the preceding clauses, or a pharmaceutically acceptable salt thereof, wherein ring A is a phenylene, m is 1, and R¹ is methyl, ethyl, F, Cl, Br, —CN,

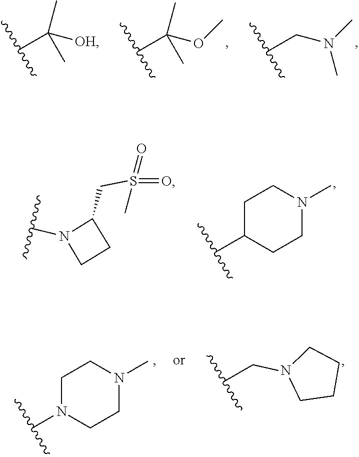

wherein each “ [symbol] ” represents a point of covalent attachment.

8. The compound of any one of the preceding clauses, or a pharmaceutically acceptable salt thereof, wherein ring A is

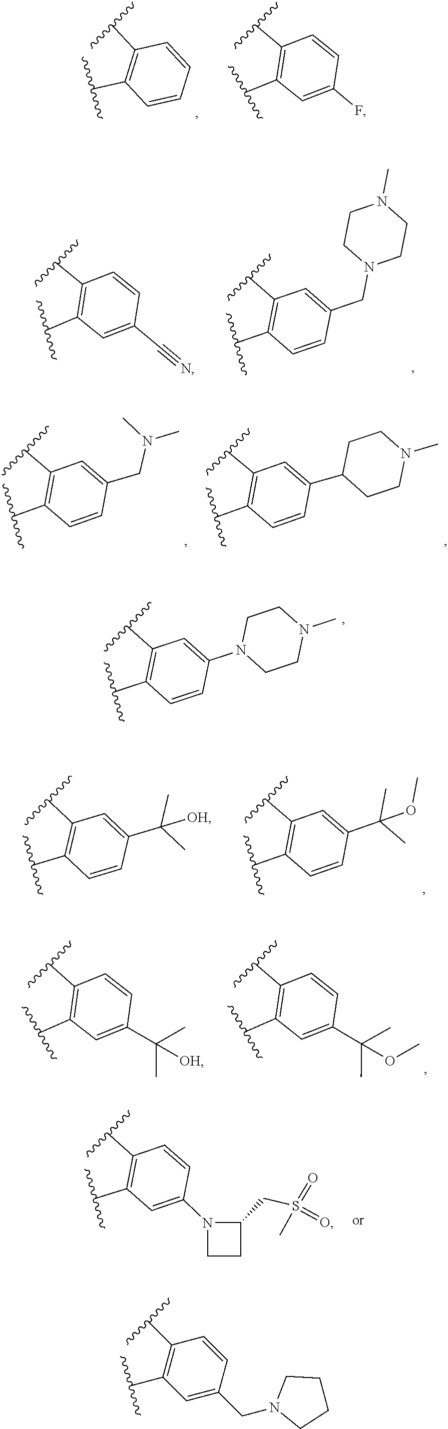

wherein each “ [symbol] ” represents a point of covalent attachment.

9. The compound of any one of clauses 1 to 4, or a pharmaceutically acceptable salt thereof, wherein ring A is a 5- to 10-membered heteroarylene, and m is 0, 1, 2, or 3.

10. The compound of any one of clauses 1 to 4, or a pharmaceutically acceptable salt thereof, wherein ring A is a 5- to 10-membered heteroarylene, and m is 0, 1, or 2.

11. The compound of any one of the clauses 1 to 4, or 10, or a pharmaceutically acceptable salt thereof, having the formula II

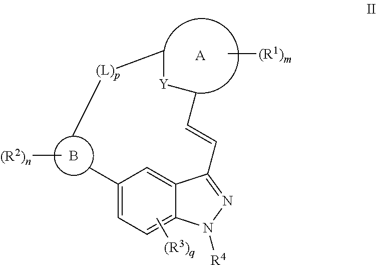

wherein Y is a single, a double bond, —O—, —S—, ═C(H)—, ═C(R⁵)—, —N(H)—, —N(R⁵)— or ═N—, and ring A is a 5- to 10-membered heteroarylene.

12. The compound of any one of clauses 1 to 4, 10 or 11, or a pharmaceutically acceptable salt thereof, having the formula III

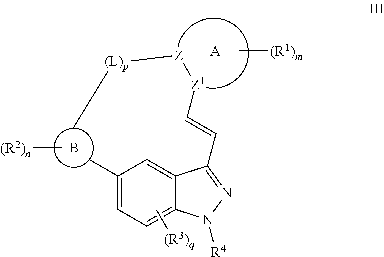

wherein Z is

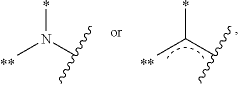

or, wherein * is a point of covalent attachment to (L)ₚ provided that * is not an N—O or N—N bond, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is or

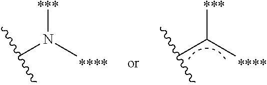

wherein *** is a point of covalent attachment to “ [symbol] ”, **** is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, provided that both Z and Z¹ are not a nitrogen atom; and ring A is a 5- to 10-membered heteroarylene.

13. The compound of any one of clauses 1 to 4, or 10 to 12, or a pharmaceutically acceptable salt thereof, having the formula IV

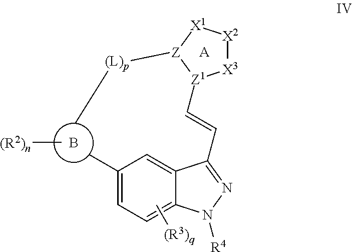

wherein
X¹, X², and X³ are each independently —O—, —S—, ═C(H)—, ═C(R⁵)—, —N(H)—, —N(R⁵)— or ═N—, provided that at least one of X¹, X², and X³ is not ═C(H)—, or ═C(R⁵)—;
Z is

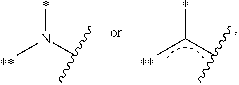

wherein * is a point of covalent attachment to (L)ₚ provided that * is not an N—O or N—N bond, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is

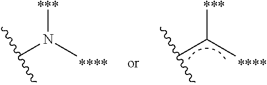

or wherein *** is a point of covalent attachment to “ [symbol] ”, **** is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, provided that both Z and Z are not a nitrogen atom; and
ring A is a 5- to 10-membered heteroarylene.

14. The compound of any one of the preceding clauses, or a pharmaceutically acceptable salt thereof, wherein ring A is a 5- to 10-membered heteroarylene selected from the group consisting of

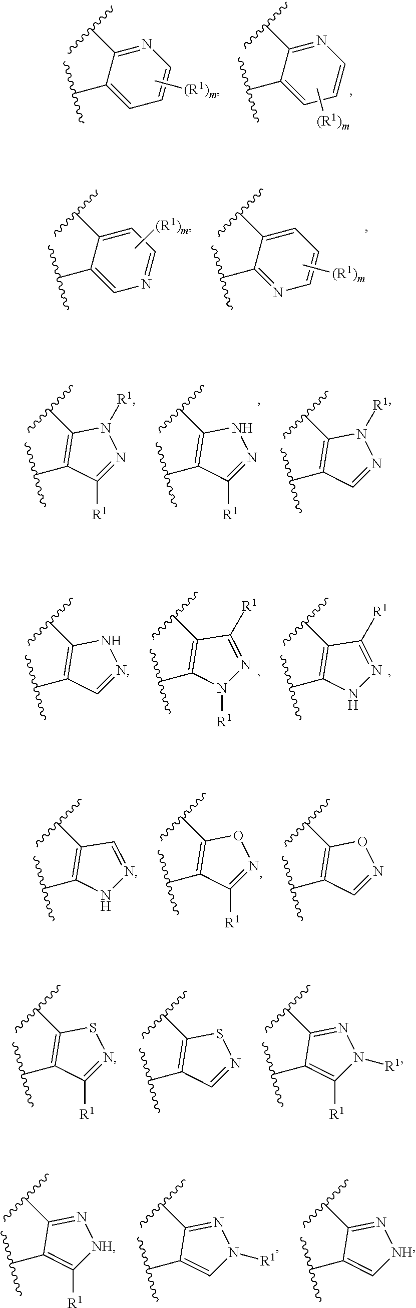

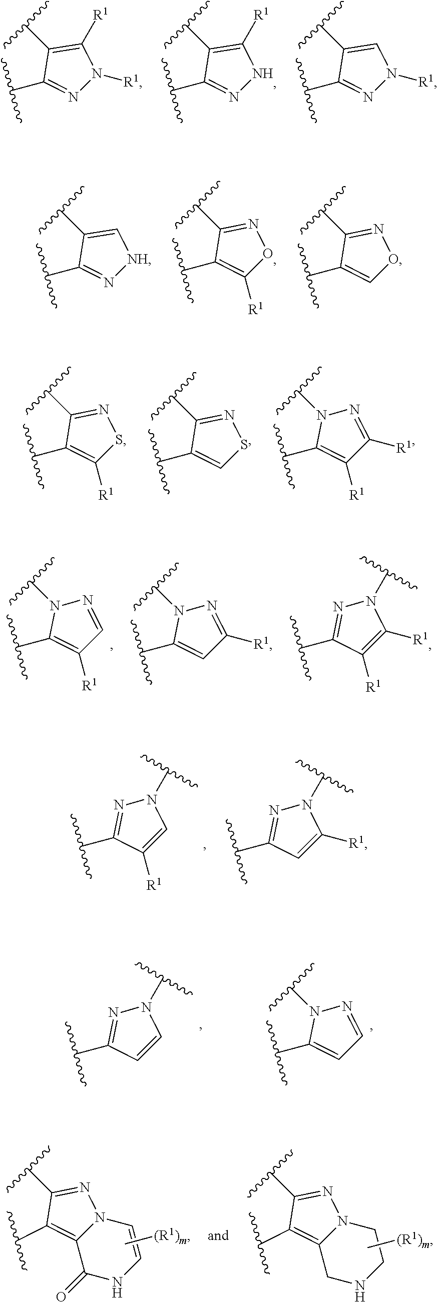

wherein each “ [symbol] ” represents a point of covalent attachment.

15. The compound of any one of the preceding clauses, or a pharmaceutically acceptable salt thereof, wherein ring A is a 5- to 10-membered heteroarylene selected from the group consisting of

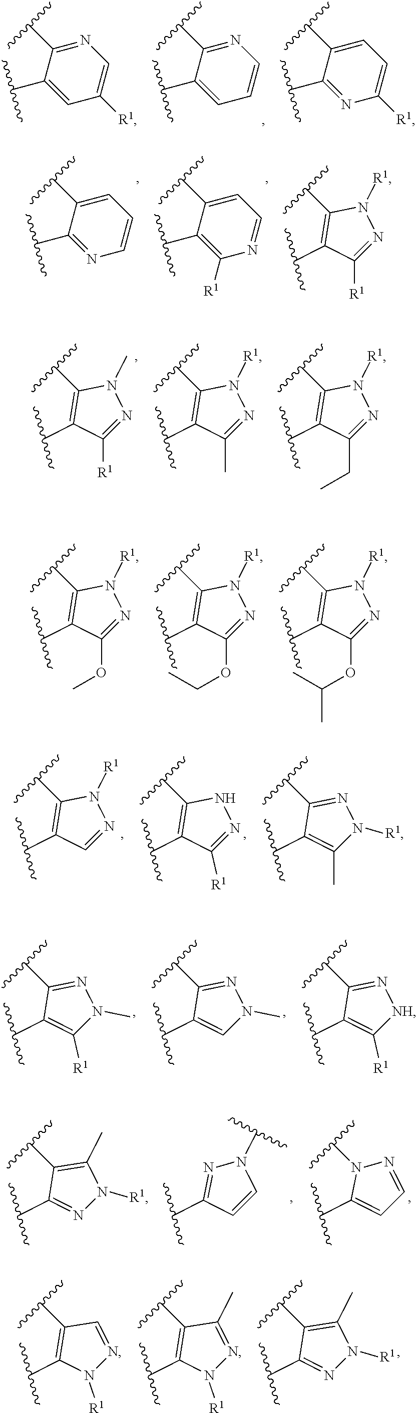

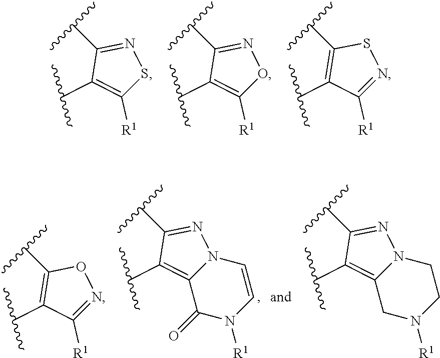

wherein each “ [symbol] ” represents a point of covalent attachment.

16. The compound of any one of the preceding clauses, or a pharmaceutically acceptable salt thereof, wherein ring A is a 5- to 10-membered heteroarylene selected from the group consisting of

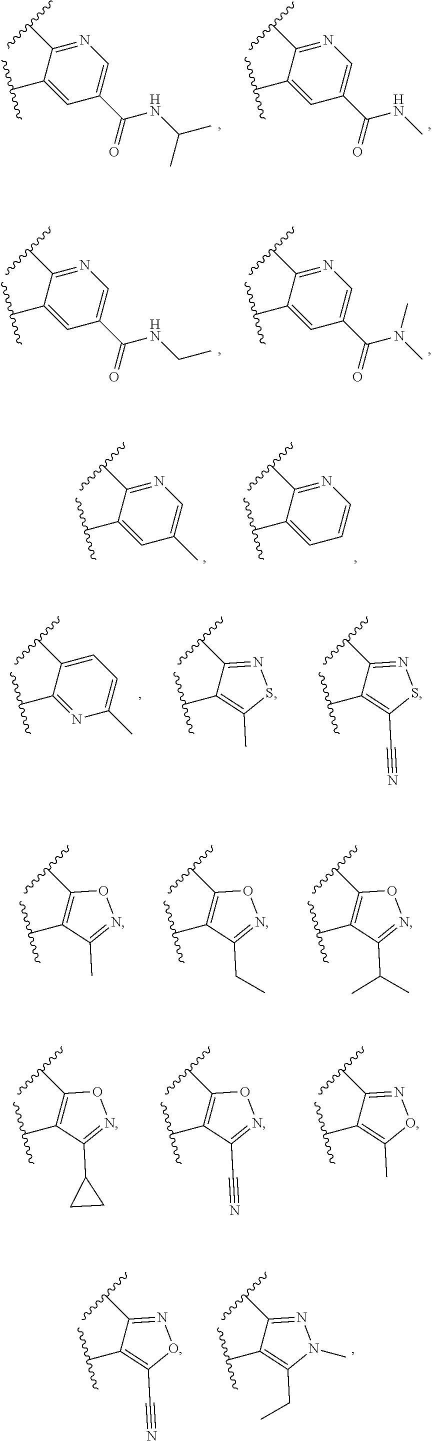

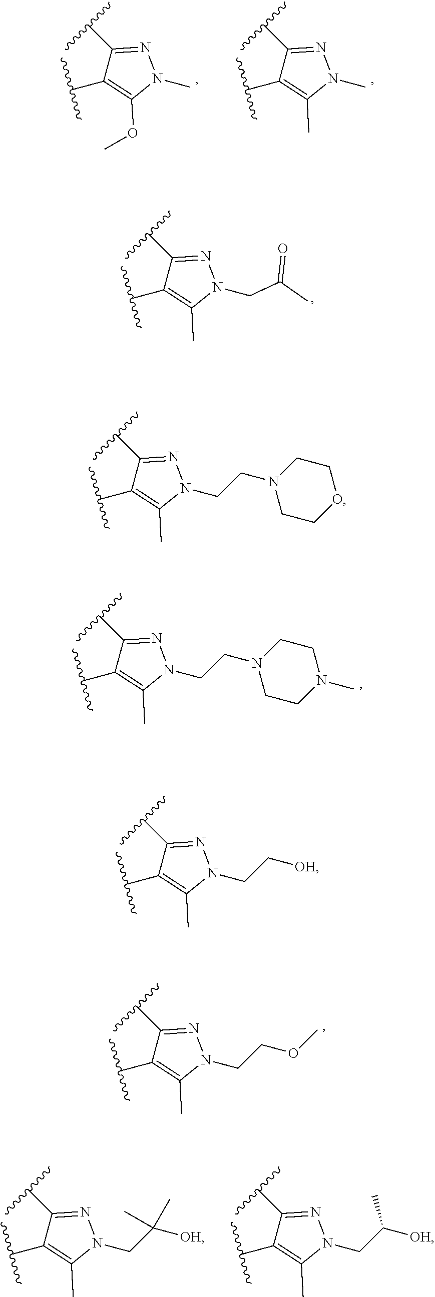

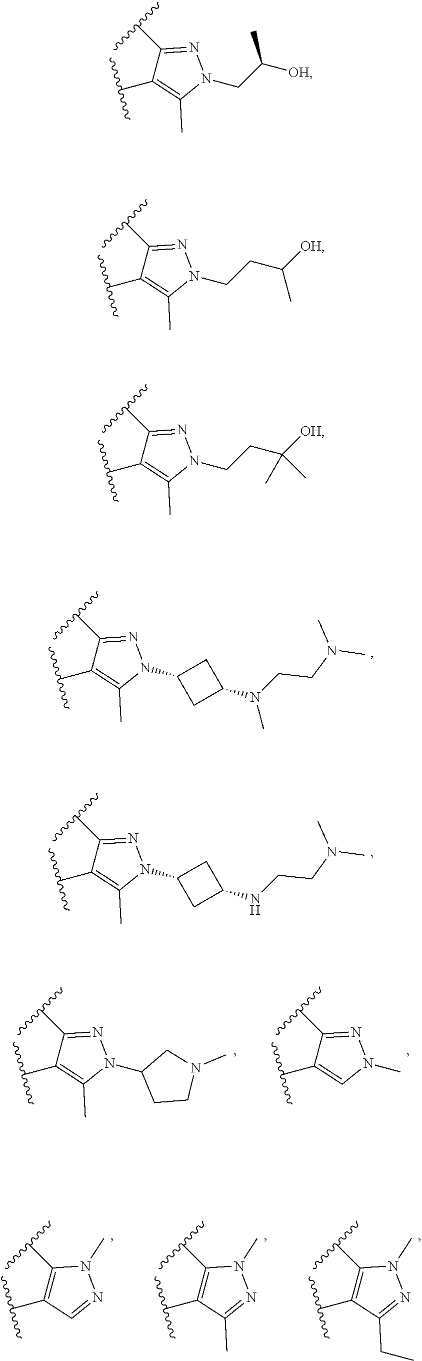

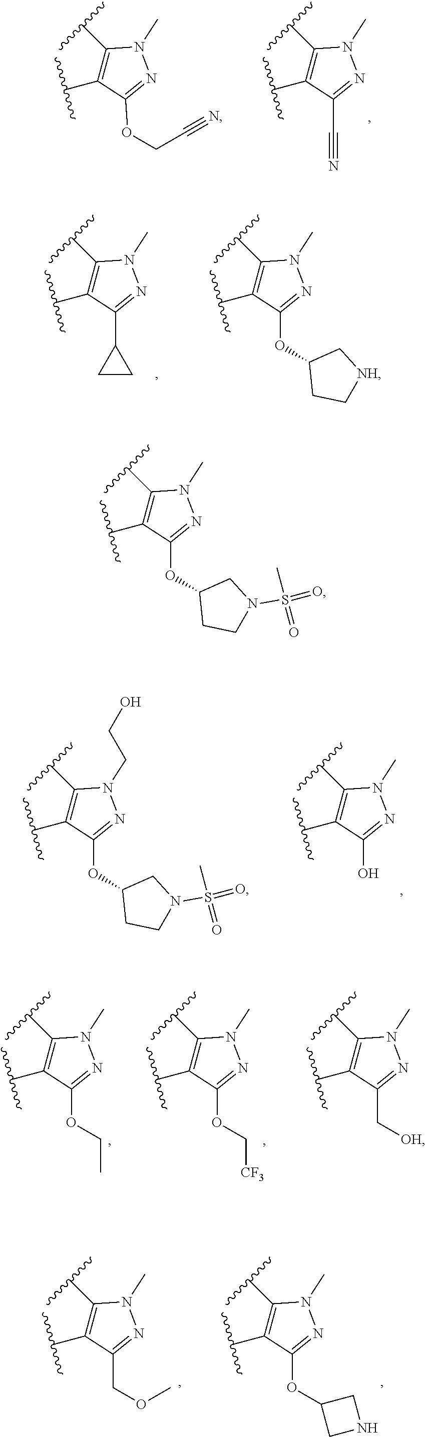

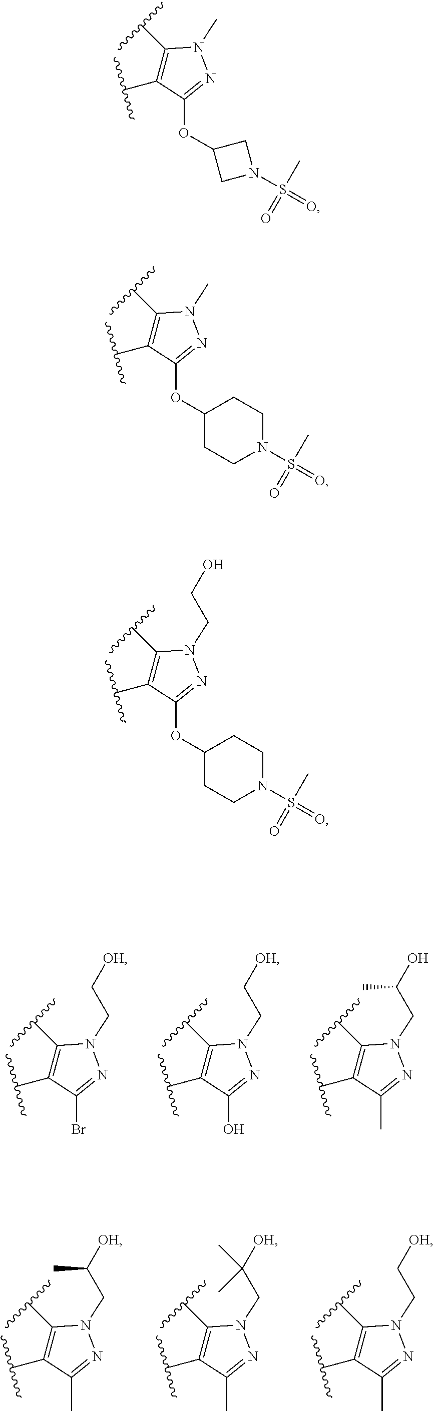

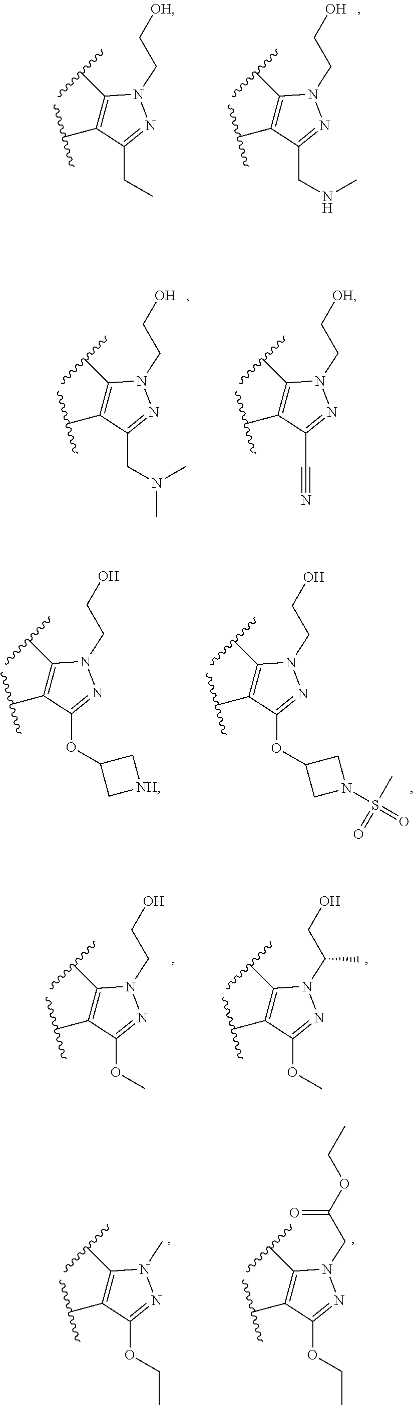

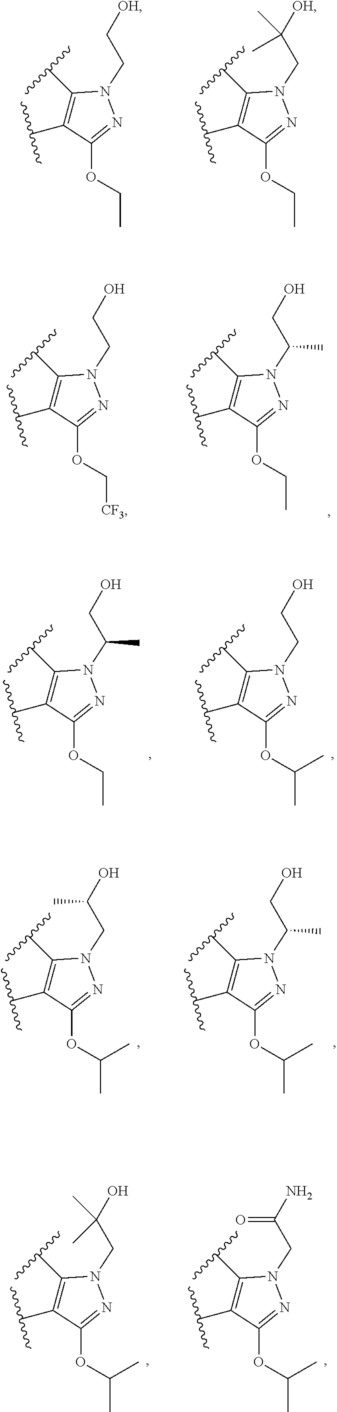

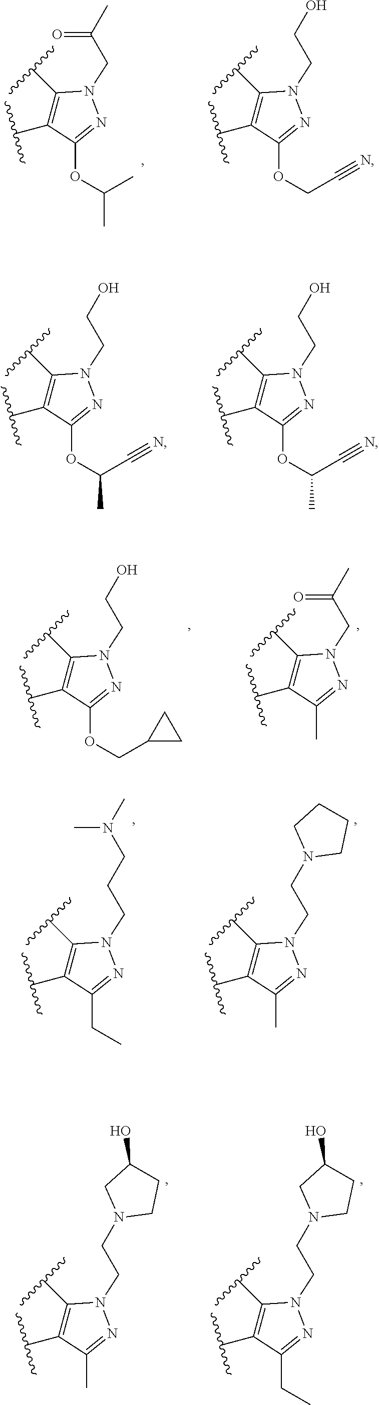

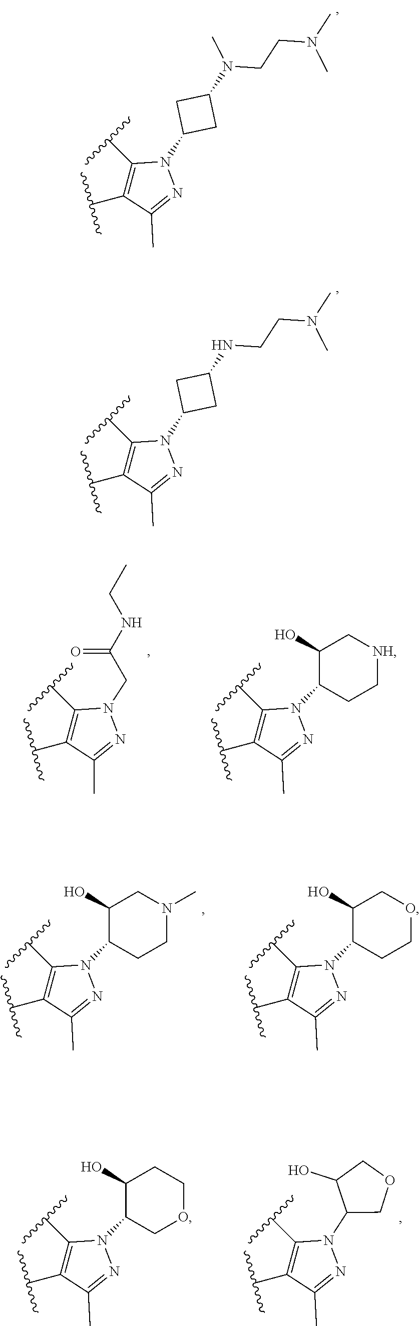

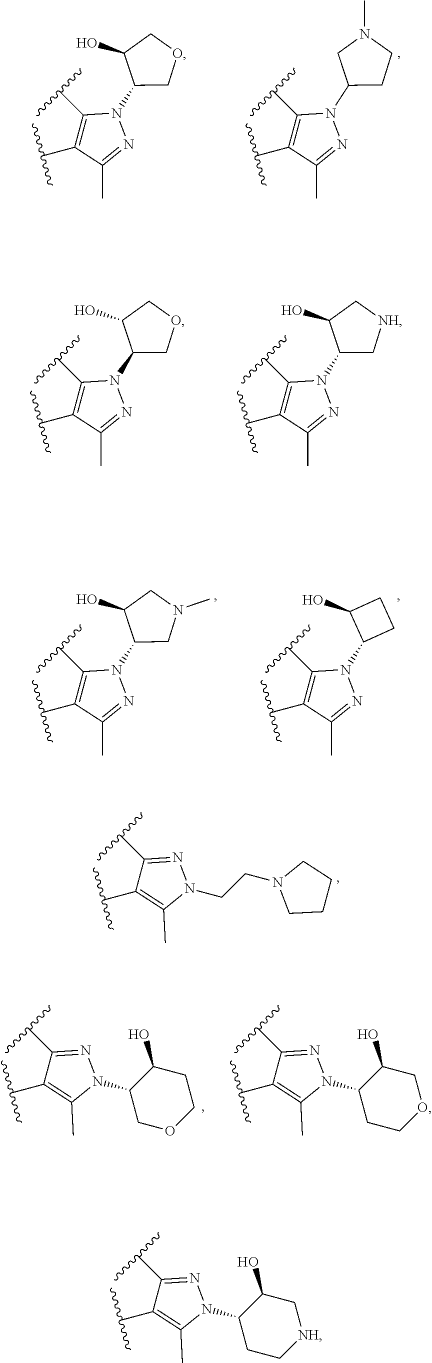

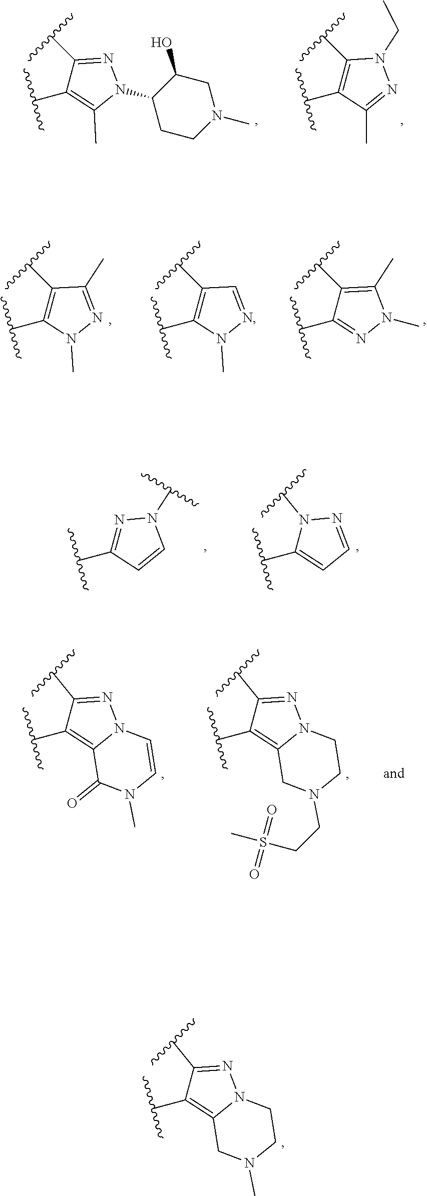

wherein each “ [symbol] ” represents a point of covalent attachment.

17. The compound of any one of the preceding clauses, or a pharmaceutically acceptable salt thereof, wherein ring B is a 5-membered heteroarylene selected from the group consisting of

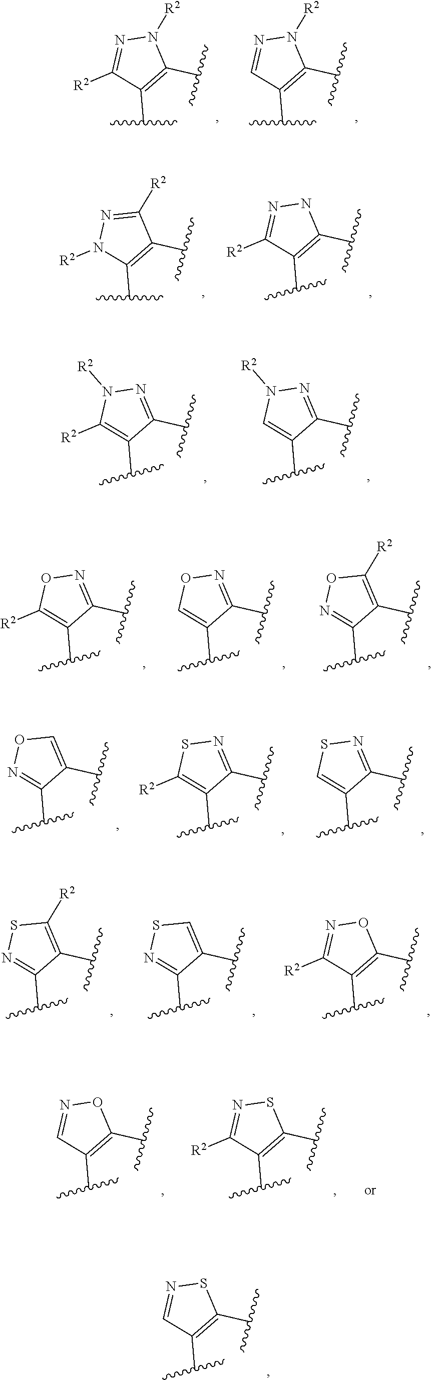

wherein each “ [symbol] ” represents a point of covalent attachment.

18. The compound of any one of the preceding clauses, or a pharmaceutically acceptable salt thereof, wherein ring B is a 5-membered heteroarylene selected from the group consisting of

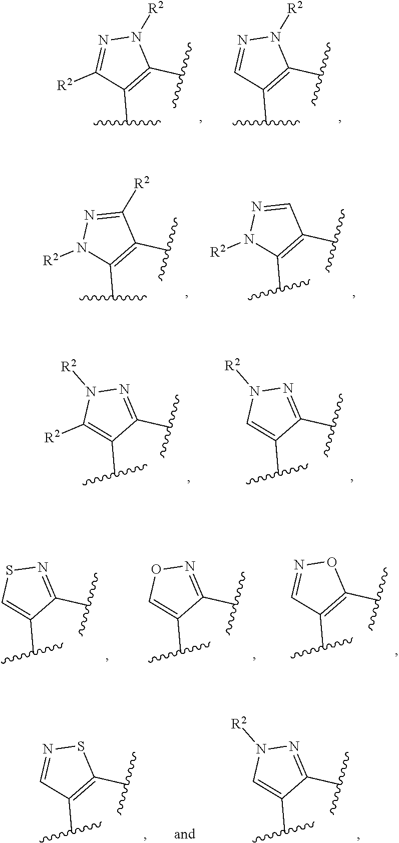

wherein each “ [symbol] ” represents a point of covalent attachment.

19. The compound of any one of the preceding clauses, or a pharmaceutically acceptable salt thereof, wherein ring B is a 5-membered heteroarylene selected from the group consisting of

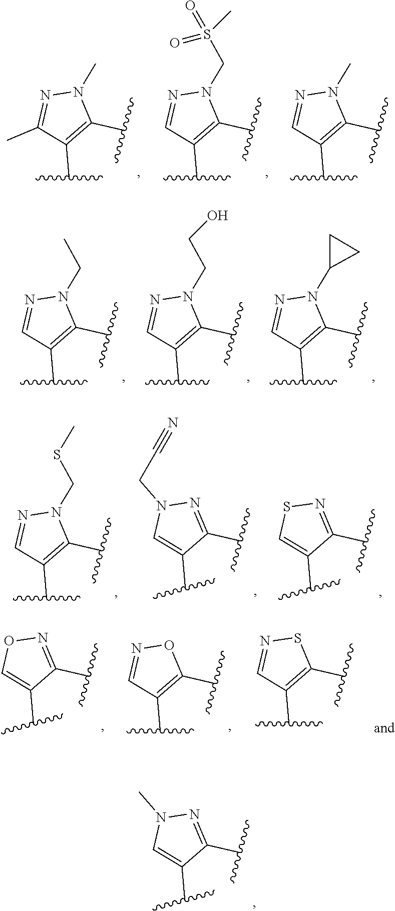

wherein each “ [symbol] ” represents a point of covalent attachment.

20. The compound of any one of the preceding clauses, or a pharmaceutically acceptable salt thereof, q is 0, 1, or 2.

21. The compound of any one of the preceding clauses, or a pharmaceutically acceptable salt thereof, q is 0 or 1.

22. The compound of any one of the preceding clauses, or a pharmaceutically acceptable salt thereof, wherein R³ is F.

23. The compound of any one of the preceding clauses, or a pharmaceutically acceptable salt thereof, wherein R⁴ is H or methyl.

24. The compound of any one of the preceding clauses, or a pharmaceutically acceptable salt thereof, wherein each L is independently —C(R⁶)(R⁷)—, —C(O)—, —O—, or —N(R⁵)—, provided that (L)ₚ does not comprise a —O—O— or a —O—N(R⁵)— bond, and the point of covalent attachment of (L)ₚ to ring A does not form a —N—N— or a —O—N— bond.

25. The compound of any one of the preceding clauses, or a pharmaceutically acceptable salt thereof, wherein -(L)ₚ- is —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂O(CR⁶R⁷)₂O—, —CR⁶R⁷O—CR⁶R⁷, —C(O)N(R⁵)—(CR⁶R⁷)₂—, —(CR⁶R⁷)₃O(CR⁶R⁷)₂—, —(CR⁶R⁷)₂O(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O—CR⁶R⁷—, —O(CR⁶R⁷)₂O(CR⁶R⁷)₂—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

26. The compound of any one of the preceding clauses, or a pharmaceutically acceptable salt thereof, wherein each R⁵ is independently H, methyl, ethyl, —C(O)CH₃, or —C(O)CH₂CH₃; or R⁵, when present, and an R⁶ or R⁷, when present, taken together with the atom or atoms to which they are attached, combine to form a 3- to 7-membered heterocycloalkyl; wherein each hydrogen atom in the 3- to 7-membered heterocycloalkyl formed when R⁵ and an R⁶ or R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{f}, —S(O)NR^{e}R^{f}, —S(O)₂NR^{e}R^{f}, —NR^{e}R^{f}, —NR^{c}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

27. The compound of any one of the preceding clauses, or a pharmaceutically acceptable salt thereof, wherein an R⁶ and R⁷, when present, taken together with the atom or atoms to which they are attached, combine to form a 3- to 7-membered heterocycloalkyl; wherein each hydrogen atom in the 3- to 7-membered heterocycloalkyl formed when R⁶ and R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

28. The compound of any one of the preceding clauses, or a pharmaceutically acceptable salt thereof, wherein each R⁶, that is not taken together with R⁵ or an R⁷, is independently H or C₁C₆ alkyl.

29. The compound of any one of the preceding clauses, or a pharmaceutically acceptable salt thereof, wherein one R⁶, that is not taken together with R⁵ or an R⁷, is methyl, and the remaining R⁶ and R⁷ are H.

30. The compound of any one of the preceding clauses, or a pharmaceutically acceptable salt thereof, wherein -(L)ₚ- is —O—(CH₂)₂O—CH₂—, —OC(H)(CH₃)—CH₂—O—CH₂—, —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —OCH₂—C(H)(CH₃)—N(CH(CH₃)₂)CH₂—, —OCH₂—C(H)(CH₂F)—N(CH₃)CH₂—, —OCH₂—C(H)(CH₂CH₃)—N(CH₃)CH₂—, —O(CH₂)₂N(C(O)CH₃)CH₂—, —OC(H)(CH₃)CH₂N(H)CH₂—, —OC(H)(CH₃)CH₂N(CH₃)CH₂—, —OC(H)(CH₂CN)CH₂N(CH₃)CH₂—, —OC(H)(CH₂CH₃)CH₂N(CH₃)CH₂—, —OC(H)(CH₂F)CH₂N(CH₃)CH₂—, —OC(H)(CHF₂)CH₂N(CH₃)CH₂—, —OC(H)(CH₂OH)CH₂N(CH₃)CH₂—, —OC(H)(CH₃)C(O)N(CH₃)CH₂—, —OC(H)(cyclobutyl)CH₂N(CH₃)CH₂—, —OC(H)(CH₃)CH₂N(CH₂CH₃)CH₂—, —OC(H)(CH₃)CH₂N(CH(CH₃)₂)—CH₂—, —OC(H)(CH₃)CH₂N(CH₃)—C(H)(CH₃)—, —OC(H)(CH₃)CH₂N(CH(oxetan-3-yl)-CH₂—, —OC(H)(CH₃)CH₂N(cyclopropyl)-CH₂—, —OCH₂C(H)(CH₃)N(cyclopropyl)-CH₂—, —OC(H)(CH₃)CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—C(H)(CH₃)—CH₂O—, —CH₂N(CH₃)—(CH₂)₂(C(H)CH₃)CH₂O—, —CH₂N(CH₃)—(CH₂)—(C(H)CH₃)O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—C(H)(CH₃)—O—, —O(CH₂)₂C(H)(CH₃)CH₂O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —OC(H)(CH₃)CH₂OCH₂—,

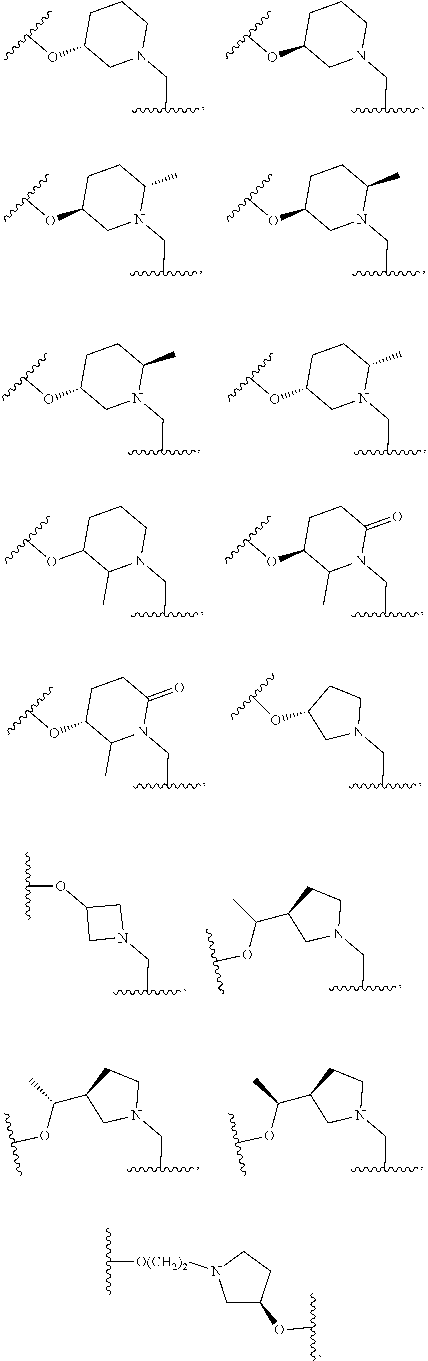

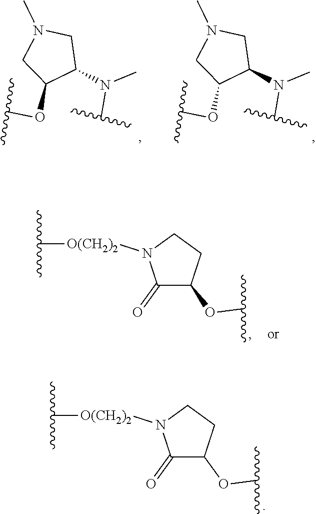

31. The compound of clause 1 or 2, selected from the group consisting of (11E)-1-methyl-1,18,19,21-tetrahydro-8H-10,7,4-(ethan[1]yl [1,2]diylidene)pyrazolo[3,4-f][1,4,12,13]benzodioxadiazacyclooctadecine;
(11E)-1-[(methanesulfonyl)methyl]-19,20-dihydro-1H,8H,18H-10,7,4-(ethan[1]yl[1,2]diylidene)pyrazolo[3,4-f][1,5,12,13]benzodioxadiazacyclooctadecine;
(11E)-1-methyl-18,19,20,21-tetrahydro-1H,8H-10,7,4-(ethan[1]yl[1,2]diylidene)pyrazolo[3,4-g][1,6,13,14]benzodioxadiazacyclononadecine;
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-[(4-methylpiperazin-1-yl)methyl]-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-[(dimethylamino)methyl]-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(1-methylpiperidin-4-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-(2-methoxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(4-methylpiperazin-1-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-fluoro-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-(2-hydroxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-{(2S)-2-[(methanesulfonyl)methyl]azetidin-1-yl}-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-25-oxo-3,5,6,9,10,19-hexahydro-8H-20,22-etheno-7,10-methanodipyrazolo[3,4-i:4′,3′-m][1,8,5]benzodioxazacyclooctadecine-14-carbonitrile; and
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecine;
or a pharmaceutically acceptable salt thereof.
32. The compound of clause 1 or 2, selected from the group consisting of (18E)-8-methyl-N-(propan-2-yl)-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N,8-dimethyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N,N,8-trimethyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N-ethyl-8-methyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(10S,18E)-8,10,16-trimethyl-2,8,11,12-tetrahydro-10H-3,5-ethenodipyrazolo[3′,4′:10,11;4″,3″:14,15][1,5]dioxacyclopentadecino[7,6-b]pyridine;
(10S,18E)-17-ethoxy-8,10,12-trimethyl-2,8,10,11,12,13-hexahydro-3,5-ethenodipyrazolo|3,4-j:4′,3′-nipyrido|4,3-f∥1,4|oxazacyclopentadecine; and
(10S,18E)-16-ethoxy-8,10,12-trimethyl-2,8,10,11,12,13-hexahydro-3,5-ethenodipyrazolo[3,4-j:4′,3′-n]pyrido[3,2-f][1,4]oxazacyclopentadecine;
or a pharmaceutically acceptable salt thereof.
33. The compound of clause 1 or 2, selected from the group consisting of (17E)-8,14,16-trimethyl-2,8,9,11,12,14-hexahydro-3,5-etheno[1,4]dioxacyclopentadecino[11,10-c:15,14-c′:6,7-c″]tripyrazole;
2-[(17E)-8,9,16-trimethyl-8,9,11,12-tetrahydro-3,5-etheno[1,4]dioxacyclopentadecino[11,10-c:15,14-c′:6,7-c″]tripyrazol-14(2H)-yl]ethan-1-ol;
(17E)-8,14,16-trimethyl-2,11,12,14-tetrahydro-8H,10H-3,5-etheno[1,5]dioxacyclopentadecino[10,11-c:15,14-c′:6,7-c″]tripyrazole;
(17E)-19-fluoro-8,14,16-trimethyl-2,11,12,14-tetrahydro-8H,10H-3,5-etheno[1,5]dioxacyclopentadecino[10,11-c:15,14-c′:6,7-c″]tripyrazole;
(17E)-16-ethyl-8,14-dimethyl-2,11,12,14-tetrahydro-8H,10H-3,5-etheno[1,5]dioxacyclopentadecino[10,11-c:15,14-c′:6,7-c″]tripyrazole;
1-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-2-methylpropan-2-ol;
1-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]propan-2-one;
2-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]ethan-1-ol;
(17E)-8,16-dimethyl-14-[2-(pyrrolidin-1-yl)ethyl]-2,11,12,14-tetrahydro-8H,10H-3,5-etheno[1,5]dioxacyclopentadecino[10,11-c:15,14-c′:6,7-c′]tripyrazole;
(3S)-1-{2-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]ethyl}pyrrolidin-3-ol;
(17E)-16-ethyl-8,12,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-8,10,14,16-tetramethyl-2,11,12,14-tetrahydro-8H,10H-3,5-etheno[1,5]dioxacyclopentadecino[10,11-c:15,14-c′:6,7-c″]tripyrazole;
(10R,17E)-8,10,14,16-tetramethyl-2,11,12,14-tetrahydro-8H,10H-3, 5-etheno[1,5]dioxacyclopentadecino[10,11-c:15,14-c′:6,7-c″]tripyrazole;
(12S,17E)-16-ethyl-8,12,14-trimethyl-2,11,12,14-tetrahydro-8H,10H-3,5-ethenol[1,5|dioxacyclopentadecinol10,11-c:15,14-c′:6,7-c″]tripyrazole;
(12R,17E)-16-ethyl-8,12,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-16-ethyl-8,10,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-16-cyclopropyl-8,10,14-trimethyl-2,11,12,14-tetrahydro-8H,10H-3,5-etheno[1,5]dioxacyclopentadecino[10,11-c:15,14-c′:6,7-c″]tripyrazole;
(17E)-16-(methoxymethyl)-8,14-dimethyl-2,11,12,14-tetrahydro-8H,10H-3,5-etheno[1,5]dioxacyclopentadecino[10,11-c:15,14-c′:6,7-c″]tripyrazole;
(17E)-8,14,16-trimethyl-2,10,11,12,13,14-hexahydro-8H-3,5-ethenotripyrazolo[3,4-f:3′,4′-j:4″,3″-n][1,4]oxazacyclopentadecine;
1-[(17E)-8,14,16-trimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one;
(17E)-8,12,14,16-tetramethyl-2,10,11,12,13,14-hexahydro-8H-3,5-ethenotripyrazolo[3,4-f:3′,4′-j:4″,3″-n][1,4]oxazacyclopentadecine;
(17E)-6,8,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(17E)-16-ethyl-8,12,14-trimethyl-2,10,11,12,13,14-hexahydro-8H-3,5-ethenotripyrazolo[3,4-f:3′,4′-j:4″,3″-n][1,4]oxazacyclopentadecine;
(17E)-7,14,16-trimethyl-2,10,11,12,13,14-hexahydro-7H-3,5-ethenotripyrazolo[3,4-f:3′,4′-j:4″,3″-n][1,4]oxazacyclopentadecine;
(17E)-7,12,14,16-tetramethyl-2,10,11,12,13,14-hexahydro-7H-3,5-ethenotripyrazolo[3,4-f:3′,4′-j:4″,3″-n][1,4]oxazacyclopentadecine;
2-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(17E)-8,14,16-trimethyl-2,9,10,11,12,14-hexahydro-8H-3,5-ethenotripyrazolo[3,4-f:4′,3′-j:4″,3″-n][1,4]oxazacyclopentadecine;
(17E)-8,10,14,16-tetramethyl-2,9,10,11,12,14-hexahydro-8H-3,5-ethenotripyrazolo[3,4-f:4′,3′-j:4″,3″-n][1,4]oxazacyclopentadecine;
2-[(17E)-8,16-dimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-10-ethyl-8,16-dimethyl-2,8,9,10,11,12-hexahydro-14H-3,5-ethenotripyrazolo|3,4-f:4′,3′-j:4″,3″-n∥1,4|oxazacyclopentadecin-14-yl|ethan-1-ol;
(19E)-8,13,16,18-tetramethyl-2,11,12,13,14,16-hexahydro-8H,10H-3,5-etheno[1,7]dioxacycloheptadecino[12,13-c:17,16-c′:8,9-c″]tripyrazole;
2-[(19E)-8,13,18-trimethyl-2,8,11,12,13,14-hexahydro-10H,16H-3,5-etheno[1,7]dioxacycloheptadecino[12,13-c:17,16-c′:8,9-c″]tripyrazol-16-yl]ethan-1-ol;
(19E)-8,16,18-trimethyl-2,11,12,16-tetrahydro-8H,10H-3,5-ethenotripyrazolo[3,4-h:3′,4′-l:⁴″,³″-p][1,7,4]dioxazacycloheptadecin-13(14H)-one;
(19E)-8,12,16,18-tetramethyl-2,11,12,16-tetrahydro-8H,10H-3,5-ethenotripyrazolo[3,4-h:3′,4′-l:4″,3″-p][1,7,4]dioxazacycloheptadecin-13(14H)-one;
(13R,19E)-8,13,16,18-tetramethyl-2,10,11,13,14,16-hexahydro-8H-3,5-etheno[1,4,7]trioxacycloheptadecino[12,13-c:17,16-c′:8,9-c″]tripyrazole;
(13S,19E)-8,13,16,18-tetramethyl-2,10,11,13,14,16-hexahydro-8H-3,5-etheno[1,4,7]trioxacycloheptadecino[12,13-c:17,16-c′:8,9-c″]tripyrazole;
2-[(19E)-8,13,18-trimethyl-2,8,10,11,13,14-hexahydro-16H-3,5-etheno[1,4,7]trioxacycloheptadecino[12,13-c:17,16-c′:8,9-c″]tripyrazol-16-yl]ethan-1-ol;
(19E)-8,13,18-trimethyl-16-[2-(pyrrolidin-1-yl)ethyl]-2,10,11,13,14,16-hexahydro-8H-3,5-etheno[1,4,7]trioxacycloheptadecino[12,13-c:17,16-c′:8,9-c′]tripyrazole;
(3S)-1-{2-[(17E)-16-ethyl-8-methyl-2,8,11,12-tetrahydro-10H,14H-3,5-etheno[1,5]dioxacyclopentadecino [10,11-c:15,14-c′:6,7-c″]tripyrazol-14-yl]ethyl}pyrrolidin-3-ol;
(19E)-22-fluoro-8,16,18-trimethyl-2,10,11,13,14,16-hexahydro-8H-3,5-etheno[1,4,7]trioxacycloheptadecino[12,13-c:17,16-c′:8,9-c′]tripyrazole;
3-[(17E)-16-ethyl-8-methyl-2,8,11,12-tetrahydro-10H,14H-3,5-etheno[1,5]dioxacyclopentadecino[l10,11-c:15,14-c′:6,7-c″]tripyrazol-14-yl]-N,N-dimethylpropan-1-amine;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]pyrrolidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-1-methylpyrrolidin-3-ol;
(3R,4S)-4-[(17E)-8,16-dimethyl-2,8,11,12-tetrahydro-10H,14H-3,5-etheno[1,5]dioxacyclopentadecino[l10,11-c:15,14-c′:6,7-c″]tripyrazol-14-yl]oxolan-3-ol;
(3S,4R)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]oxolan-3-ol;
(rac)-2-|(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]cyclobutan-1-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]piperidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-1-methylpiperidin-3-ol;
(rac)-1,5-anhydr-3o-2,3-dideoxy-3-[(17E)-8,16-dimethyl-2,8,11,12-tetrahydro-10H,14H-3,5-etheno[1,5]dioxacyclopentadecino[10,11-c:15,14-c′:6,7-c′]tripyrazol-14-yl]-L-threo-pentitol;
(rac)-1,5-anhydro-2,4-dideoxy-2-[(17E)-8,16-dimethyl-2,8,11,12-tetrahydro-10H,14H-3,5-etheno[l1,5]dioxacyclopentadecino[10,11-c:15,14-c′:6,7-c′]tripyrazol-14-yl]-L-threo-pentitol;
(12R,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(12S,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(rac)-(3S,4S)-1-methyl-4-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]pyrrolidin-3-ol;
(11S,17E)-8,11,14,16-tetramethyl-2,9,10,11,12,14-hexahydro-8H-3,5-ethenotripyrazolo[3,4-f:4′,3′-j:4″,3″-n][1,4]oxazacyclopentadecine;
(17E)-8,9,16-trimethyl-14-[2-(pyrrolidin-1-yl)ethyl]-2,8,9,11,12,14-hexahydro-3,5-etheno[1,4]dioxacyclopentadecino[11,10-c:15,14-c′:6,7-c″]tripyrazole;
[(10S,17E)-8,10,14-trimethyl-2,11,12,14-tetrahydro-8H,10H-3,5-etheno[1,5]dioxacyclopentadecino[10,11-c:15,14-c′:6,7-c″]tripyrazol-16-yl]methanol;
N¹—{(1s,3s)-3-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]cyclobutyl}-N¹,N²—,N²-trimethylethane-1,2-diamine;
(11S,17E)-8,10,11,14,16-pentamethyl-2,9,10,11,12,14-hexahydro-8H-3,5-ethenotripyrazolo[3,4-f:4′,3′-j:4″,3″-n][1,4]oxazacyclopentadecine;
N²—{(1s,3s)-3-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]cyclobutyl}-N¹,N¹-dimethylethane-1,2-diamine;
(10R,15E)-3,12,14-trimethyl-5,6,9,10,12,18-hexahydro-3H,8H-19,21-etheno-7,10-methanotripyrazolol3,4-i:3′,4′-m:4″,3″-q∥1,8,4|dioxazacyclooctadecin-24-one;
(17E)-16-ethyl-8,10,14-trimethyl-2,9,10,11,12,14-hexahydro-8H-3,5-ethenotripyrazolo[3,4-f:4′,3′-j:4″,3″-n][1,4]oxazacyclopentadecine;
(17E)-10-ethyl-8,14,16-trimethyl-2,9,10,11,12,14-hexahydro-8H-3,5-ethenotripyrazolo[3,4-f:4′,3′-j:4″,3″-n][1,4]oxazacyclopentadecine;
2-[(10S,17E)-8,10,16-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-19-fluoro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(17E)-8,10,16-trimethyl-14-(1-methylpyrrolidin-3-yl)-2,9,10,11,12,14-hexahydro-8H-3,5-ethenotripyrazolo[3,4-f:4′,3′-j:4″,3″-n][1,4]oxazacyclopentadecine;
2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(10R,19E)-8,16,18-trimethyl-2,8,10,11,12,13,15,16-octahydro-3,5-etheno-10,14-methanotripyrazolo[3,4-h:3′,4′-l:4″,3″-p][1, 6]oxazacycloheptadecine;
(2S)-1-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]propan-2-ol;
2-[(10S,17E)-19-chloro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2R)-1-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]propan-2-ol;
2-[(10S,17E)-16-ethyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-ethyl-8,10,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14-tetramethyl-16-[(piperidin-4-yl)oxy]-2,10,11,12,13,14-hexahydro-8H-3,5-ethenotripyrazolo[3,4-f:3′,4′-j:4″,3″-n][1,4]oxazacyclopentadecine;
2-[(10R,19E)-18-ethoxy-8-methyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(10S,17E)-6,8,10,12,16-pentamethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,14,16-tetramethyl-12-(oxetan-3-yl)-2,10,11,12,13,14-hexahydro-8H-3,5-ethenotripyrazolo|3,4-f:3′,4′-j:4″,3″-n∥1,4|oxazacyclopentadecine;
2-[(10S,17E)-16-ethoxy-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-{[1-(methanesulfonyl)piperidin-4-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,13R,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
(10S,13S,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
2-{(10S,17E)-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-[(azetidin-3-yl)oxy]-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-16-{[1-(methanesulfonyl)azetidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
2-1[(10S,17E)-16-ethoxy-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(17E)-10-cyclobutyl-8,12,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14-tetramethyl-16-{[(3S)-pyrrolidin-3-yl]oxy}-2,10,11,12,13,14-hexahydro-8H-3,5-ethenotripyrazolo[3,4-f:3′,4′-j:4″,3″-n][1,4]oxazacyclopentadecine;
(10S,17E)-16-{1[(3S)-1-(methanesulfonyl)pyrrolidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(2S)-2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(10S,17E)-16-ethoxy-8,10,14-trimethyl-12-(propan-2-yl)-2,10,11,12,13,14-hexahydro-8H-3,5-ethenotripyrazolo[3,4-f:3′,4′-j:4″,3″-n][1,4]oxazacyclopentadecine;
(10S,17E)-8,10,14-trimethyl-2,11,12,14-tetrahydro-8H,10H-3,5-etheno|1,5|dioxacyclopentadecino|10,11-c:15,14-c′:6,7-c″|tripyrazole-16-carbonitrile;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10R,19E)-18-ethoxy-8,16-dimethyl-2,8,10,11,12,13,15,16-octahydro-3,5-etheno-10,14-methanotripyrazolo[3,4-h:3′,4′-l:4″,3″-p][1,6]oxazacycloheptadecine;
(10S,17E)-12-cyclopropyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-8,10,12,14-tetramethyl-16-(2,2,2-trifluoroethoxy)-2,10,11,12,13,14-hexahydro-8H-3,5-ethenotripyrazolo[3,4-f:3′,4′-j:4″,3″-n][1,4]oxazacyclopentadecine;
{[(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
(10S,17E)-10,14,16-trimethyl-8-[(methylsulfanyl)methyl]-12-(propan-2-yl)-2,10,11,12,13,14-hexahydro-8H-3,5-ethenotripyrazolo[3,4-f:3′,4′-j:4″,3″-n][11,4]oxazacyclopentadecine;
(10S,17E)-12-ethyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-8-ethyl-10,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-8-ethyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2R)-2-[(1S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-19-fluoro-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-{[1-(methanesulfonyl)piperidin-4-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,19E)-8,16,18-trimethyl-2,8,10,11,12,13,15,16-octahydro-3,5-etheno-10,14-methanotripyrazolo[3,4-h:3′,4′-l:4″,3″-p][1,6]oxazacycloheptadecine;
2-[(1S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10S,17E)-16-{[(3S)-1-(methanesulfonyl)pyrrolidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-bromo-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-16-[(azetidin-3-yl)oxy]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-({[3-(methanesulfonyl)cyclobutyl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
(10R,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
2-[(10R,19E)-18-ethoxy-22-fluoro-8-methyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(11S,17E)-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-12-cyclopropyl-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-|(10R,17E)-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(11S,17E)-12-cyclopropyl-16-ethoxy-8,11-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10R,19E)-22-fluoro-8-methyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
(2S)-2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(11S,17E)-6,8,11-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-6,8,10-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
{[(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-cyclopropyl-16-ethoxy-6,8,10-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(11S,17E)-12-cyclopropyl-16-ethoxy-6,8,11-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-ethoxy-12-ethyl-19-fluoro-8,10,14-trimethyl-2,10,11,12,13,14-hexahydro-8H-3,5-ethenotripyrazolo[3,4-f:3′,4′-j:4″,3″-n][1,4]oxazacyclopentadecine;
(2S)-2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-i4-yl}propan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10S,17E)-12-ethyl-8,10,14-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
2-[(1S,17E)-19-fluoro-8,10,12-trimethyl-16-(2,2,2-trifluoroethoxy)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10R,17E)-12-cyclopropyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-12-cyclopropyl-8,11-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2S)-2-[(10S,17E)-16-ethoxy-19-fluoro-8,10,12-trimethyl-2,8,10,11,12,13-hexahydro-14H-3,5-ethenotripyrazolo[3,4f:3′,4′-j:4″,3″-n][1,4]oxazacyclopentadecin-14-yl]propan-1-ol;
(2S)-2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-t-ol;
2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
1-[(10R,17E)-16-ethoxy-14-(2-hydroxyethyl)-8,10-dimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one;
ethyl [(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]acetate;
(2S)-2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(10S,17E)-14-(2-hydroxyethyl)-8,10,12-trimethyl-16-[(propan-2-yl)oxy]-2,12,13,14-tetrahydro-8H-3,5-ethenotripyrazolo[3,4-f:3′,4′-j:4″,3″-n][1,4]oxazacyclopentadecin-11(10H)-one;
2-[(10S,17E)-16-{[1-(methanesulfonyl)azetidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
1-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]-2-methylpropan-2-ol;
2-{(10S,13R,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,13S,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-10-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-11-(fluoromethyl)-8,12-dimethyl-16-[(propan-2-yl)oxy]-2,8,10,11,12,13-hexahydro-14H-3,5-ethenotripyrazolo[3,4f:3′,4′-j:4″,3″-n][1,4]oxazacyclopentadecin-14-yl}ethan-1-ol;
(2S)-1-{(10R,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
(2S)-2-[(11S,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-[(10R,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(2S)-2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}acetamide;
(10S,17E)-19-fluoro-14-(2-hydroxyethyl)-8,10,12-trimethyl-2-(propan-2-yl)-2,10,11,12,13,14-hexahydro-8H-3,5-ethenotripyrazolo[3,4-f:3′,4′-j:4″,3″-n][1,4]oxazacyclopentadecin-16-ol;
2-[(8aR,19E)-1-(cyclopropylmethoxy)-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
(10R,18E)-17-ethoxy-8,15-dimethyl-2,8,11,12,14,15-hexahydro-10H-3,5-etheno-10,13-methanotripyrazolo[3,4-g:3′,4′-k:4″,3″-o][1,5]oxazacyclohexadecine;
{(17E)-14-(2-hydroxyethyl)-12-(propan-2-yl)-16-[(propan-2-yl)oxy]-2,10,11,12,13,14-hexahydro-7H-3,5-ethenotripyrazolo[3,4-f:3′,4′-j:4″,3″-n][1,4]oxazacyclopentadecin-7-yl}acetonitrile;
(17E)-16-ethoxy-8,14-dimethyl-2,8,10,11,13,14-hexahydro-3,5-etheno-10,12-methanotripyrazolo[3,4-f:3′,4′-j:4″,3″-n][1,4]oxazacyclopentadecine;
2-[(10S,17E)-16-ethoxy-19-fluoro-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(8aR,9R,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-22-fluoro-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(10S,17E)-8-cyclopropyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10S,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
2-{(10R,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(8aR,9S,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9R,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-22-fluoro-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
—{(10R,17E)-10-(hydroxymethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11S,17E)-16-ethoxy-11-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-10-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-(methoxymethyl)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
1-{(10S,17E)-12-ethyl-8,10-dimethyl-16-|(propan-2-yl)oxyl-2,8,10,11,12,13-hexahydro-14H-3,5-ethenotripyrazolo[3,4-f:3′,4′-j:4″,3″-n][1,4]oxazacyclopentadecin-14-yl}-2-methylpropan-2-ol;
1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-one;
(10R,17E)-14-(2-hydroxyethyl)-8,12-dimethyl-16-[(propan-2-yl)oxy]-2,10,11,12,13,14-hexahydro-8H-3,5-ethenotripyrazolo[3,4-f:3′,4′-j:4″,3″-n][1,4]oxazacyclopentadecine-10-carbonitrile;
2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-16-[(dimethylamino)methyl]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol:
(2R)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
(2S)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
2-[(4aS,7aS,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
2-[(4aR,7aR,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
{[(8aR,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
{[(8aR,9R,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
2-{(10S,17E)-10-(difluoromethyl)-8,12-dimethyl-16-|(propan-2-yl)oxyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(4aS,7aS,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(4aR,7aR,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(2S)-1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-{(11R,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11R,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-12-ethyl-19-fluoro-16-methoxy-8,10-dimethyl-2,8,10,11,12,13-hexahydro-14H-3,5-ethenotripyrazolo[3,4f:3′,4′-j:4″,3″-n][1,4]oxazacyclopentadecin-14-yl]propan-1-ol;
(10S,17E)-16-ethoxy-12-ethyl-14-(2-hydroxyethyl)-8,10-dimethyl-2,10,11,12,13,14-hexahydro-8H-3,5-ethenotripyrazolo[3,4-f:3′,4′-j:4″,3″-n][1,4]oxazacyclopentadecine-6-carbonitrile;
2-[(10S,17E)-16-ethoxy-6-ethynyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-(ethylamino)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-1-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-[(10S,17E)-6-amino-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2,2′-[(10S,17E)-10,12-dimethyl-16-[(propan-2-yl)oxy]-10,11,12,13-tetrahydro-8H-3,5-ethenotripyrazolo[3,4-f:3′,4′-j:4″,3″-n][1,4]oxazacyclopentadecine-8,14(2H)-diyl]di(ethan-1-ol);
(17E)-16-ethyl-8,14-dimethyl-2,8,10,11,13,14-hexahydro-3,5-etheno[1,4]dioxacyclopentadecino[10,11-c:15,14-c′:6,7-c″ ]tripyrazole;
2-[(17E)-8,16-dimethyl-2,10,11,13-tetrahydro-3,5-etheno[1,4]dioxacyclopentadecino[10,11-c:15,14-c′:6,7-c″]tripyrazol-14(8H)-yl]-N-ethylacetamide;
(17E)-8,14-dimethyl-2,8,10,11,13,14-hexahydro-3,5-etheno[1,4]dioxacyclopentadecino[10,11-c:15,14-c′:6,7-c″ ]tripyrazole-16-carbonitrile;
(17E)-8,14,16-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]thiazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]thiazole-16-carbonitrile; and
(10S,17E)-16-ethyl-8,10,14-trimethyl-2,8,10,11,13,14-hexahydro-3,5-etheno[1,4]dioxacyclopentadecino[10,11-c:15,14-c′:6,7-c″ ]tripyrazole;
or a pharmaceutically acceptable salt thereof.
34. The compound of clause 1 or 2, selected from the group consisting of (17E)-8,15,16-trimethyl-2,8,9,11,12,15-hexahydro-3,5-etheno[1,4]dioxacyclopentadecino[11,10-c:15,14-c′:6,7-c′]tripyrazole;
(17E)-8,15,16-trimethyl-2,11,12,15-tetrahydro-8H,10H-3,5-etheno[1,5]dioxacyclopentadecino[10,11-c:15,14-c′:6,7-c″ ]tripyrazole;
1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]propan-2-one;
1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-[5-yl]-2-methylpropan-2-ol;
2-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]ethan-1-ol;
(17E)-8,16-dimethyl-15-[2-(pyrrolidin-1-yl)ethyl]-2,11,12,15-tetrahydro-8H,10H-3,5-etheno[1,5]dioxacyclopentadecino[10,11-c:15,14-c′:6,7-c′]tripyrazole;
(10S,17E)-8,10,15,16-tetramethyl-2,10,11,12,13,15-hexahydro-8H-3,5-ethenotripyrazolo[3,4-f:3′,4′-j:4″,3″-n][1,4]oxazacyclopentadecine;
(17E)-8,15,16-trimethyl-2,10,11,12,13,15-hexahydro-8H-3,5-ethenotripyrazolo[3,4-f:3′,4′-j:4″,3″-n][1,4]oxazacyclopentadecine;
1-[(17E)-8,15,16-trimethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(17E)-8,12,15,16-tetramethyl-2,10,11,12,13,15-hexahydro-8H-3,5-ethenotripyrazolo[3,4-f:3′,4′-j:4″,3″-n][1,4]oxazacyclopentadecine;
1-[(10S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(10S,17E)-8,10,12,15,16-pentamethyl-2,10,11,12,13,15-hexahydro-8H-3,5-ethenotripyrazolo[3,4-f:3′,4′-j:4″,3″-n][1,4]oxazacyclopentadecine;
2-[(10S,17E)-8,10,16-trimethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
2-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
(10S,17E)-12-ethyl-8,10,15,16-tetramethyl-2,10,11,12,13,15-hexahydro-8H-3,5-ethenotripyrazolo[3,4-f:3′,4′-j:4″,3″-n][1,4]oxazacyclopentadecine;
1-[(10S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]propan-1-one;
2-[(19E)-8,13,18-trimethyl-2,8,11,12,13,14-hexahydro-10H,17H-3,5-etheno[1,7]dioxacycloheptadecino[12,13-c:17,16-c′:8,9-c″ ]tripyrazol-17-yl]ethan-1-ol;
(13E)-3-methyl-3,5,6,7,8,16-hexahydro-9,12-(azeno)-17,19-ethenodipyrazolo[3,4-1:4′,3′-p][1,6]oxazacycloheptadecine;
(18E)-8-methyl-2,8,10,11,12,13-hexahydro-3,5-ethenotripyrazolo[1,5-f:3′,4′-j:4″,3″-n][1,6]oxazacyclopentadecine;
(17E)-15-(2-methoxyethyl)-8,16-dimethyl-2,11,12,15-tetrahydro-8H,10H-3,5-etheno[1,5]dioxacyclopentadecino[10,11-c:15,14-c′:6,7-c″ ]tripyrazole;
2-[(17E)-8,9,16-trimethyl-8,9,11,12-tetrahydro-3,5-etheno[1,4]dioxacyclopentadecino[11,10-c:15,14-c′:6,7-c″]tripyrazol-15(2H)-yl]ethan-1-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]piperidin-3-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-1-methylpiperidin-3-ol;
(17E)-8,9,16-trimethyl-15-[2-(pyrrolidin-1-yl)ethyl]-2,8,9,11,12,15-hexahydro-3,5-etheno[1,4]dioxacyclopentadecino[11,10-c:15,14-c′:6,7-c″ ]tripyrazole;
(rac)-1,5-anhydro-2,3-dideoxy-3-[(17E)-8,16-dimethyl-2,8,11,12-tetrahydro-10H,15H-3,5-etheno[1,5]dioxacyclopentadecino[10,11-c:15,14-c′:6,7-c′]tripyrazol-15-yl]-L-threo-pentitol;
1,5-anhydro-2,4-dideoxy-2-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-L-threo-pentitol;
(10S,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(10S,17E)-8,10,12,16-tetramethyl-15-[2-(4-methylpiperazin-1-yl)ethyl]-2,10,11,12,13,15-hexahydro-8H-3,5-ethenotripyrazolo[3,4-f:3′,4′-j:4″,3″-n][1,4]oxazacyclopentadecine;
(20E)-8,18-dimethyl-2,8,11,12-tetrahydro-10H-3,5-ethenodipyrazolo[3″,4″:10′,11′;4′″,3′″:14′,15′][1,5]dioxacyclopentadecino[6′,7′:3,4]pyrazolo[1, 5-a]pyrazin-19(18H)-one;
(10S,17E)-8,10,12,16-tetramethyl-15-[2-(morpholin-4-yl)ethyl]-2,10,11,12,13,15-hexahydro-8H-3,5-ethenotripyrazolo[3,4-f:3′,4′-j:4″,3″-n][1,4]oxazacyclopentadecine;
4-[(1S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
N¹—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]cyclobutyl}-N¹,N²—,N²-trimethylethane-1,2-diamine;
2-methyl-4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
2-methyl-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
N²—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]cyclobutyl}-N¹,N¹-dimethylethane-1,2-diamine;
(17E)-8,10,16-trimethyl-15-(1-methylpyrrolidin-3-yl)-2,9,10,11,12,15-hexahydro-8H-3,5-ethenotripyrazolo[3,4-f:4′,3′-j:4″,3″-n][1,4]oxazacyclopentadecine;
(10R,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(2S)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,15-pentamethyl-2,10,11,12,13,15-hexahydro-8H-3,5-ethenotripyrazolo[4,3-f:3′,4′-j:4″,3″-n][1,4]oxazacyclopentadecine;
(2R)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,16-pentamethyl-2,10,11,12,13,16-hexahydro-8H-3,5-ethenotripyrazolo[4,3-f:3′,4′-j:4″,3″-n][1,4]oxazacyclopentadecine;
(10S,17E)-8,10,12,16-tetramethyl-2,8,10,11,12,13-hexahydro-3,5-etheno[1,2]oxazolo[3,4-f]dipyrazolo[3,4-j:4′,3′-n][1,4]oxazacyclopentadecine;
(10S,17E)-8,10,12,16-tetramethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12,16-tetramethyl-2,8,10,11,12,13-hexahydro-3,5-ethenodipyrazolo[3,4-j:4′,3′-n][1,2]thiazolo[3,4-f][1,4]oxazacyclopentadecine;
(10S,20E)-18-[2-(methanesulfonyl)ethyl]-8,10,12-trimethyl-2,8,10,11,12,13,16,17,18,19-decahydro-3,5-ethenopyrazino[1′,2′:1,5]pyrazolo[3,4-f]dipyrazolo[3,4-j:4′,3′-n][1,4]oxazacyclopentadecine;
(10S,17E)-16-cyclopropyl-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethyl-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethoxy-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10R,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]oxazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole-16-carbonitrile;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]thiazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]thiazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]oxazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]thiazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]thiazole-16-carbonitrile;
(17E)-8,16-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole;
(17E)-8,16-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole; and
(17E)-8,16-dimethyl-2,8,10,11-tetrahydro-13H-3,5-ethenodipyrazolo[3′,4′:10,11;4″,3″:14,15][1,4]dioxacyclopentadecino[6,7-c][1,2]thiazole;
or a pharmaceutically acceptable salt thereof.

35. A pharmaceutical composition comprising a compound of any one of the preceding clauses, and optionally one or more excipients.

36. A method of treating disease in a subject comprising, administering a therapeutically effective amount of a compound of any one of clauses 1 to 34, or a pharmaceutical composition of clause 35.

37. A compound according to any one of clauses 1 to 34, for use in a method of treating disease in a subject.

38. Use of a compound according to any one of clauses 1 to 34 in the manufacture of a medicament for the treatment of disease in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “*”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by

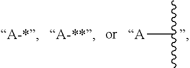

where each of “-*”, “-**”, and

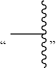

represents a bond to A and the point of covalent bond attachment to B. Alternatively, in some embodiments, the portion of A-B defined by the group or chemical structure B can be represented by

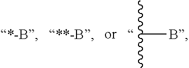

where each of “-*”, “-**”, and

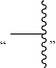

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. The term “alkenylene” refers to a straight- or branched-chain di-valent hydrocarbon group having one or more double bonds. In some embodiments, it can be advantageous to limit the number of atoms in an “alkenyl” or “alkenylene” to a specific range of atoms, such as C₂-C₂₀ alkenyl or C₂-C₂₀ alkenylene, C₂-C₁₂ alkenyl or C₂-C₁₂ alkenylene, or C₂-C₆ alkenyl or C₂-C₆ alkenylene. Examples of alkenyl groups include ethenyl (or vinyl), allyl, and but-3-en-1-yl. Examples of alkenylene groups include ethenylene (or vinylene) (—CH═CH—), n-propenylene (—CH═CHCH₂—), iso-propenylene (—CH═CH(CH₃)—), and the like. Included within this term are cis and trans isomers and mixtures thereof. It will be appreciated that an alkenyl or alkenylene group can be unsubstituted or substituted as described herein. An alkenyl or alkenylene group can be substituted with any of the substituents in the various embodiments described herein, including one or more of such substituents.

The term “alkynyl” refers to a straight- or branched-chain monovalent hydrocarbon group having one or more triple bonds. The term “alkynylene” refers to a straight- or branched-chain divalent hydrocarbon group having one or more triple bonds. In some embodiments, it can be advantageous to limit the number of atoms in an “alkynyl” or “alkynylene” to a specific range of atoms, such as C₂-C₂₀ alkynyl or C₂-C₂₀ alkynylene, C₂-C₁₂ alkynyl or C₂-C₁₂ alkynylene, or C₂-C₆ alkynyl or C₂-C₆ alkynylene. Examples of alkynyl groups include acetylenyl (—C≡CH) and propargyl (—CH₂C≡CH), but-3-yn-1,4-diyl (—C≡C≡CH₂CH₂—), and the like. It will be appreciated that an alkynyl or alkynylene group can be unsubstituted or substituted as described herein. An alkynyl or alkynylene group can be substituted with any of the substituents in the various embodiments described herein, including one or more of such substituents.

The term “cycloalkyl” refers to a saturated or partially saturated, monocyclic or polycyclic mono-valent carbocycle. The term “cycloalkylene” refers to a saturated or partially saturated, monocyclic or polycyclic divalent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include monovalent radicals of the following entities, while cycloalkylene groups include divalent radicals of the following entities, in the form of properly bonded moieties:

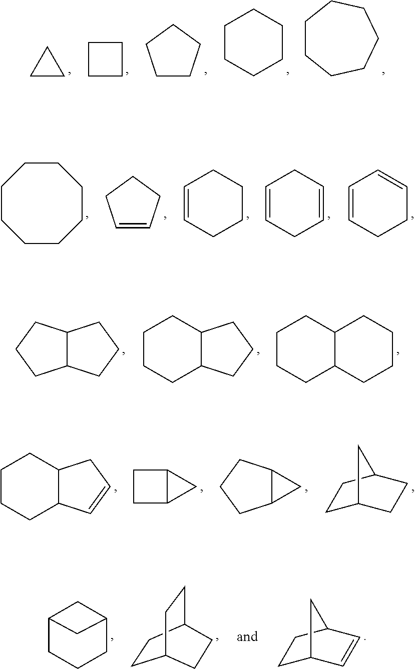

In particular, a cyclopropyl moiety can be depicted by the structural formula

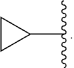 [SMILES: *C1CC1]

In particular, a cyclopropylene moiety can be depicted by the structural formula

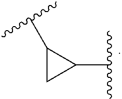 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents.

Examples of haloalkyl groups include —CF₃, —(CH₂)F, —CHF₂, —CH₂Br, —CH₂CF₃, and —CH₂CH₂F. The term “haloalkylene” refers to an alkyl group with one or more halo substituents. Examples of haloalkyl groups include —CF₂—, —C(H)(F)—, —C(H)(Br)—, —CH₂CF₂—, and —CH₂C(H)(F)—.

The term “aryl” refers to a monovalent all-carbon monocyclic or fused-ring polycyclic group having a completely conjugated pi-electron system. The term “arylene” refers to a divalent all-carbon monocyclic or fused-ring polycyclic group having a completely conjugated pi-electron system. In some embodiments, it can be advantageous to limit the number of atoms in an “aryl” or “arylene” to a specific range of atoms, such as mono-valent all-carbon monocyclic or fused-ring polycyclic groups of 6 to 14 carbon atoms (C₆-C₁₄ aryl), monovalent all-carbon monocyclic or fused-ring polycyclic groups of 6 to 10 carbon atoms (C₆-C₁₀ aryl), divalent all-carbon monocyclic or fused-ring polycyclic groups of 6 to 14 carbon atoms (C₆-C₁₄ arylene), divalent all-carbon monocyclic or fused-ring polycyclic groups of 6 to 10 carbon atoms (C₆-C₁₀ arylene). Examples, without limitation, of aryl groups are phenyl, naphthalenyl and anthracenyl. Examples, without limitation, of arylene groups are phenylene, naphthalenylene and anthracenylene. It will be appreciated that an aryl or arylene group can be unsubstituted or substituted as described herein. An aryl or arylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a divalent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group or an imino group on a carbon ring member or up to two oxo groups on sulfur ring members. Illustrative examples of heterocycloalkyl groups include monovalent radicals of the following entities, while heterocycloalkylene groups include divalent radicals of the following entities, in the form of properly bonded moieties:

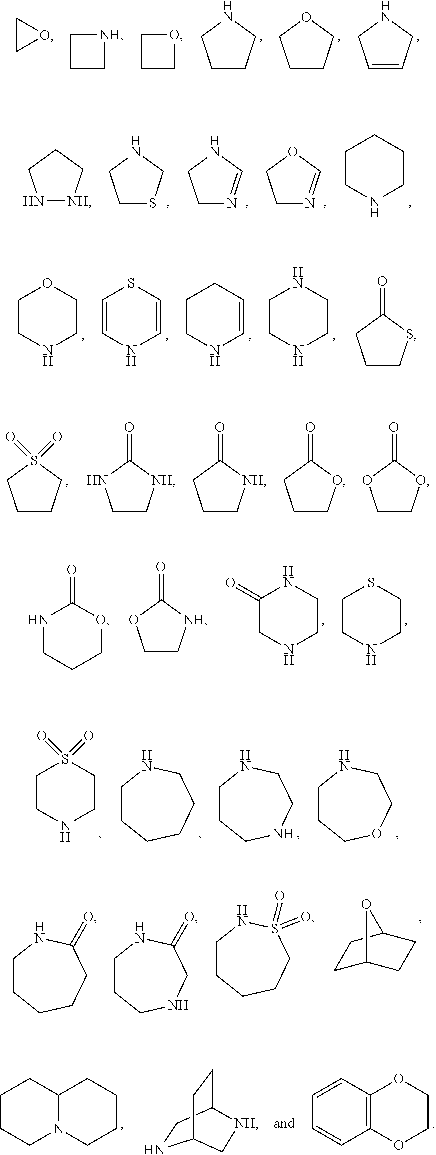

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2, 5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a divalent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5- to 10-ring atoms wherein at least one ring atom is a heteroatom, such as N, O, or S. The ring structure may optionally contain an oxo group or an imino group on a carbon ring member or up to two oxo groups on sulfur ring members. Illustrative examples of 5- to 10-membered heteroaryl groups include monovalent radicals of the following entities, while examples of 5- to 10-membered heteroarylene groups include divalent radicals of the following entities, in the form of properly bonded moieties:

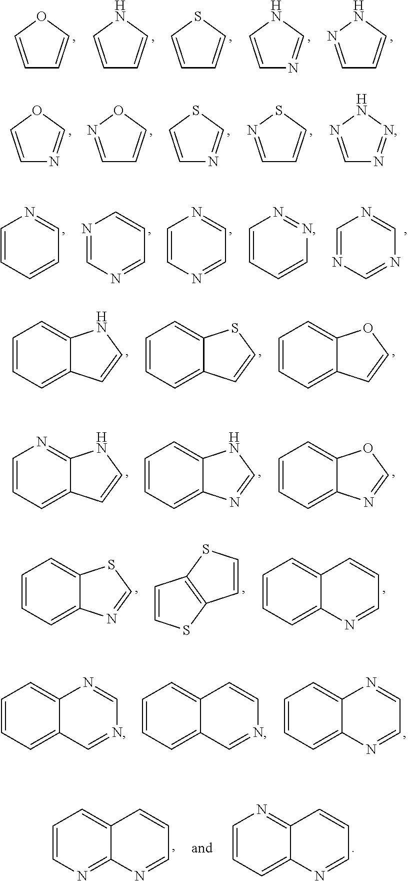

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. Non-limiting examples of six-membered heteroarylene groups include divalent radicals of pyridine, pyrazine, pyrimidine, pyridazine, or triazine. A “bicyclic heteroaryl” or “bicyclic heteroarylene” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole. Non-limiting examples of bicyclic heteroarylene groups include divalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole.

In particular, a pyrazolyl moiety can be depicted by the structural formula

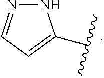 [SMILES: *c1ccn[nH]1]

In particular, an example of a pyrazolylene moiety can be depicted by the structural formula

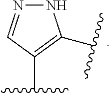 [SMILES: *c1cn[nH]c1*]

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Certain chemical entities of Formula (I)-(XII) may be depicted in two or more tautomeric forms. Any and all alternative tautomers are included within the scope of these formulas, and no inference should be made as to whether the chemical entity exists as the tautomeric form in which it is drawn. It will be understood that certain chemical entities described herein can exist in different tautomeric forms. It will be readily appreciated by one of skill in the art that because of rapid interconversion, tautomers can generally be considered to be the same chemical compound. Examples of tautomers include but are not limited to enol-keto tautomers, amine-imine tautomers, and the like.

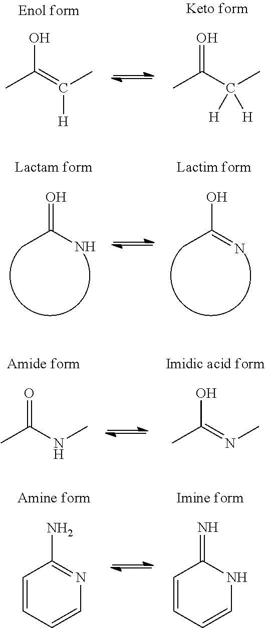

The nomenclature “(ATOM)ᵢ-(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃)

Any disubstituent referred to herein is meant to encompass the various attachment possibilities when more than one of such possibilities are allowed. For example, reference to disubstituent -J-K—, where J≠K, refers herein to such disubstituent with J attached to a first substituted member and K attached to a second substituted member, and it also refers to such disubstituent with J attached to the second substituted member and K attached to the first substituted member.

It will be appreciated that certain of the compounds described herein include one or more position that can exists as stereoisomers. For example, certain of the compounds described herein include one or more carbon atoms that can exist in one or more stereoisomeric arrangements. It will be appreciated that a carbon atom that can exist in stereoisomeric arrangements that is depicted without showing any stereoisomeric arrangement includes as a disclosure each of the possible stereoisomeric arrangements. For example a carbon atom having four groups that can be prioritized according to the Cahn-Ingold Prelog Rules known to one of skill in the art will be understood herein as describing no particular stereochemical definition as in the structure on the left below, and also as describing both possible stereoisomers (S) and (R) as shown below

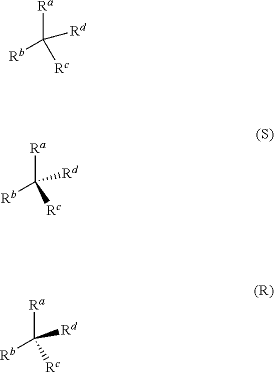

where R^{a}>R^{b}>R^{c}>R^{d} according to the Cahn-Ingold Prelog Rules.

The disclosure also includes pharmaceutically acceptable salts of the compounds represented by Formula (I)-(XII), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(XII) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)-(XII), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)-(XII)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs,” ed. H. Bundgaard, Elsevier, 1985.

The present disclosure also relates to pharmaceutically active metabolites of compounds of Formula (I)-(XII), and uses of such metabolites in the methods of the disclosure. A “pharmaceutically active metabolite” means a pharmacologically active product of metabolism in the body of a compound of Formula (I)-(XII) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

### REPRESENTATIVE EMBODIMENTS

In some embodiments, the disclosure provides a compound of the formula I, or a pharmaceutically acceptable salt thereof,

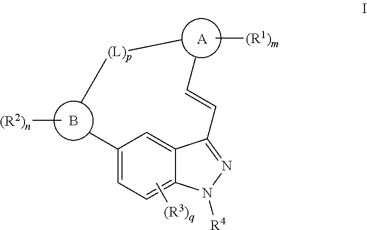

wherein R¹, R², R³, R⁴, A, B, L, m, n, p, and q are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula II, or a pharmaceutically acceptable salt thereof,

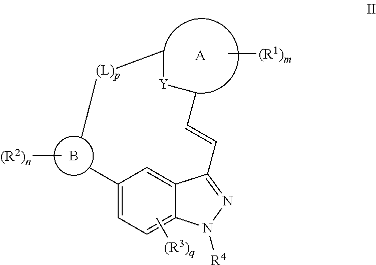

wherein R¹, R², R³, R⁴, A, B, L, Y, m, n, p, and q are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula III, or a pharmaceutically acceptable salt thereof,

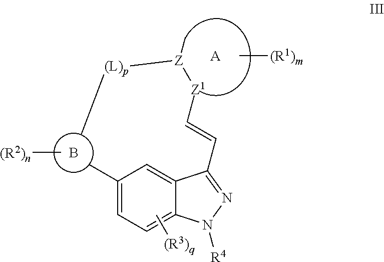

wherein R¹, R², R³, R⁴, A, B, L, Z, Z¹, m, n, p, and q are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula IV, or a pharmaceutically acceptable salt thereof,

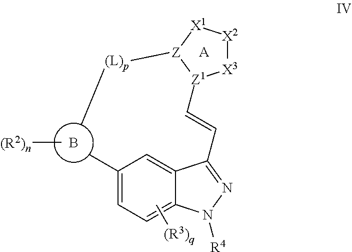

wherein R², R³, R⁴, A, B, L, X¹, X², X³, Z, Z¹, m, n, p, and q are as described herein.

In some embodiments of this aspect, (L)ₚ does not comprise a —NR⁵C(O)— directly covalently attached to ring A. In some embodiments of this aspect, (L)ₚ does not comprise a —O—CR⁶R⁷-fragment directly covalently to ring A.

In some embodiments, the disclosure provides a compound of the formula V, or a pharmaceutically acceptable salt thereof,

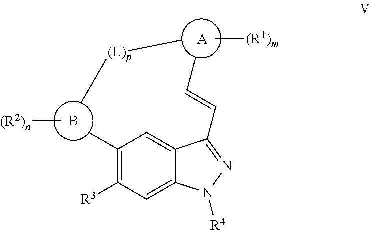

wherein R¹, R², R³, R⁴, A, B, L, m, n, and p are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula VI, or a pharmaceutically acceptable salt thereof,

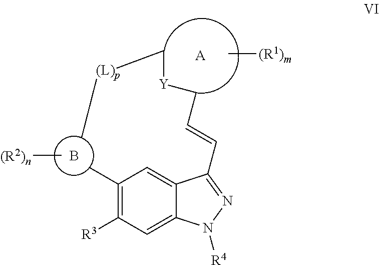

wherein R¹, R², R³, R⁴, A, B, L, Y, m, n, and p are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula VII, or a pharmaceutically acceptable salt thereof,

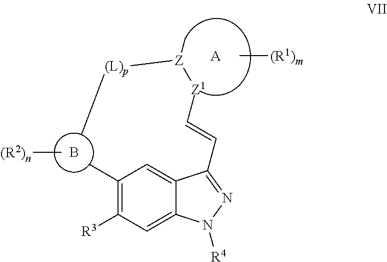

wherein R¹, R², R³, R⁴, A, B, L, Z, Z¹, m, n, and p are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷—fragment directly covalently to ring A.

In some embodiments, the disclosure provides a compound of the formula VIII, or a pharmaceutically acceptable salt thereof,

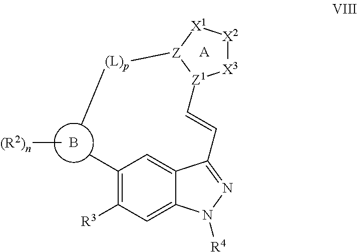

wherein R², R³, R⁴, A, B, L, X¹, X², X³, Z, Z¹, m, n, and p are as described herein. In some embodiments of this aspect, (L)ₚ does not comprise a —NR⁵C(O)— directly covalently attached to ring A. In some embodiments of this aspect, (L)ₚ does not comprise a —O—CR⁶R⁷-fragment directly covalently to ring A.

In some embodiments, the disclosure provides a compound of the formula IX, or a pharmaceutically acceptable salt thereof,

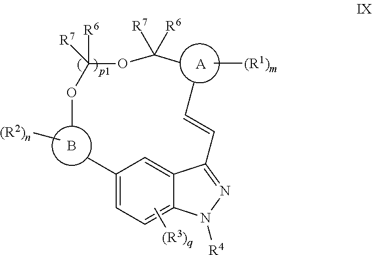

wherein R¹, R², R³, R⁴, R⁶, R⁷, A, B, m, n, p1, and q are as described herein.

In some embodiments, the disclosure provides a compound of the formula X, or a pharmaceutically acceptable salt thereof,

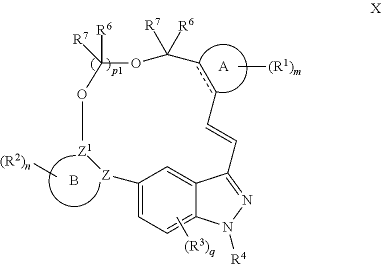

wherein R¹, R², R³, R⁴, R⁶, R⁷, A, B, Z, Z¹, m, n, p1, and q are as described herein.

In In some embodiments, the disclosure provides a compound of the formula XI, or a pharmaceutically acceptable salt thereof,

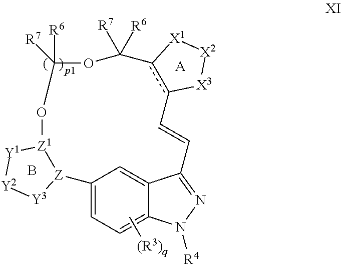

wherein R¹, R², R³, R⁴, R⁶, R⁷, A, B, X¹, X², X³, Y¹, Y², Y³, Z, Z¹, p1, and q are as described herein.

In some embodiments, the disclosure provides a compound of the formula XII, or a pharmaceutically acceptable salt thereof,

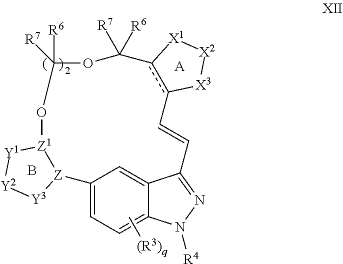

wherein R³, R⁴, R⁶, R⁷, A, B, X¹, X², X³, Y¹, Y², Y³ Z, Z¹, and q are as described herein.

In some embodiments, ring B is a 5- to 10-membered heteroarylene or a C₆-C₁₀ arylene. In some embodiments, Ring B is mono- or bi-cyclic C₆-C₁₀ arylene or mono- or bi-cyclic 5- to 10-membered heteroarylene.

In some embodiments, ring A is a 5- to 10-membered heteroarylene. In some embodiments, ring A is a 5- or 6-membered heteroarylene. In some embodiments, ring A is a 5-membered heteroarylene. In some embodiments, ring A is a 6-membered heteroarylene. In some embodiments, ring A is a fused bicyclic 8- to 10-membered heteroarylene.

In some embodiments, ring A is a 5- to 10-membered heteroarylene, such as a monocyclic 5- or 6-membered heteroarylene or a bicyclic 8- to 10-membered heteroarylene, wherein each hydrogen atom in the 5- to 10-membered heteroarylene, as described herein, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is pyrrolylene, isoxazolylene, isothiazolylene, pyrazolylene, or imidazolylene, wherein each hydrogen atom in pyrrolylene, isoxazolylene, isothiazolylene, pyrazolylene, and imidazolylene, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl. C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR′, —CN, or —NO₂.

In some embodiments, ring A is pyridinylene, pyrazinylene, pyrimidinylene, or pyridazineylene, wherein each hydrogen atom in pyridinylene, pyrazinylene, pyrimidinylene, or pyridazineylene, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is a 5- to 10-membered heteroarylene, such as a monocyclic 5- or 6-membered heteroarylene or a bicyclic 8- to 10-membered heteroarylene, wherein the 5- to 10-membered heteroarylene, as described herein, is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, and —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{c}R^{d}, —OC(═N)NR^{c}R^{d}, —OS(O)R^{e}, —OS(O)₂R^{c}, —OS(O)NR^{c}R^{d}, —OS(O)₂NR^{c}R^{d}, —SR^{c}, —S(O)RC, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, two R¹ taken together with the atoms to which they are attached combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is pyridinylene, pyrazinylene, pyrimidinylene, or pyridazineylene, wherein each is optionally substituted with 1, 2, 3, 4, or 5 of R¹ (m of R¹), each of which is independently selected from the group consisting of deuterium, halogen, C₁-C₈alkyl, C₂-C₅ alkenyl, C₂-C₅ alkynyl, C₃-C₈ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{e}, —S(O)R^{e}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, ring A is pyridinylene, pyrazinylene, pyrimidinylene, or pyridazineylene, wherein two R¹ in pyridinylene, pyrazinylene, pyrimidinylene, or pyridazineylene, as described herein, taken together with the atoms to which they are attached combine to form a C₃-C₅ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{c}, —P(O)₂OR^{e}, —CN, or —NO₂.

In some embodiments, ring A is pyrrolylene, isoxazolylene, isothiazolylene, pyrazolylene, or imidazolylene, wherein each is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, and —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{c}, —OC(O)NR^{c}R^{d}, —OC(═N)NR^{c}R^{d}, —OS(O)R^{c}, —OS(O)₂R^{e}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{e}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{c}, —CN, or —NO₂. In some embodiments, two R¹ in pyrrolylene, isoxazolylene, isothiazolylene, pyrazolylene, or imidazolylene, as described herein, taken together with the atoms to which they are attached combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is of the formula

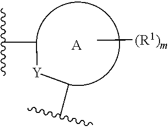

wherein Y is a single, a double bond, —O—, —S—, ═C(H)—, ═C(R¹)—, —N(H)—, —N(R¹)— or ═N—, each “ [symbol] ” represents a point of covalent attachment, and R¹ and m are as described herein. In some embodiments, ring A is 5- to 10-membered heteroarylene. In some embodiments, ring A is a 5- or 6-membered heteroarylene.

In some embodiments, ring A is of the formula

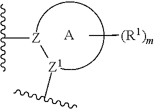

wherein Z is

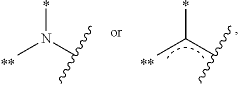

wherein * is a point of covalent attachment to (L)ₚ, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is

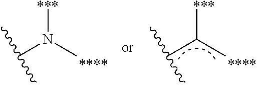

wherein *** is a point of covalent attachment to “ [symbol] ”, **** is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, and provided that both Z and Z¹ are not a nitrogen atom.

In some embodiments, ring A is 5- to 10-membered heteroarylene. In some embodiments, ring A is a 5- or 6-membered heteroarylene.

In some embodiments, ring A is of the formula

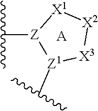

wherein
X¹, X², and X³ are each independently —O—, —S—, ═C(H)—, ═C(R⁵)—, —N(H)—, —N(R⁵)— or ═N—, provided that at least one of X¹, X², and X³ is not ═C(H)—, or ═C(R⁵)—;
Z is

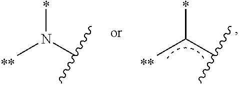

wherein * is a point of covalent attachment to (L)ₚ, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is

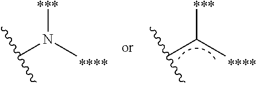

or wherein *** is a point of covalent attachment to “ [symbol] ”, **** is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, and provided that both Z and Z¹ are not a nitrogen atom. In some embodiments, ring A is 5- to 10-membered heteroarylene. In some embodiments, ring A is a 5- or 6-membered heteroarylene

In some embodiments, m is 0, 1, 2, 3, or 4. In some embodiments, m is 0, 1, 2, or 3. In some embodiments, m is 0, 1, or 2. In some embodiments, m is 0 or 1. In some embodiments, m is 0. In some embodiments, m is 1. In some embodiments, m is 2. In some embodiments, m is 3. In some embodiments, m is 4.

In some embodiments, ring A is a 5- to 10-membered heteroarylene selected from the group consisting of

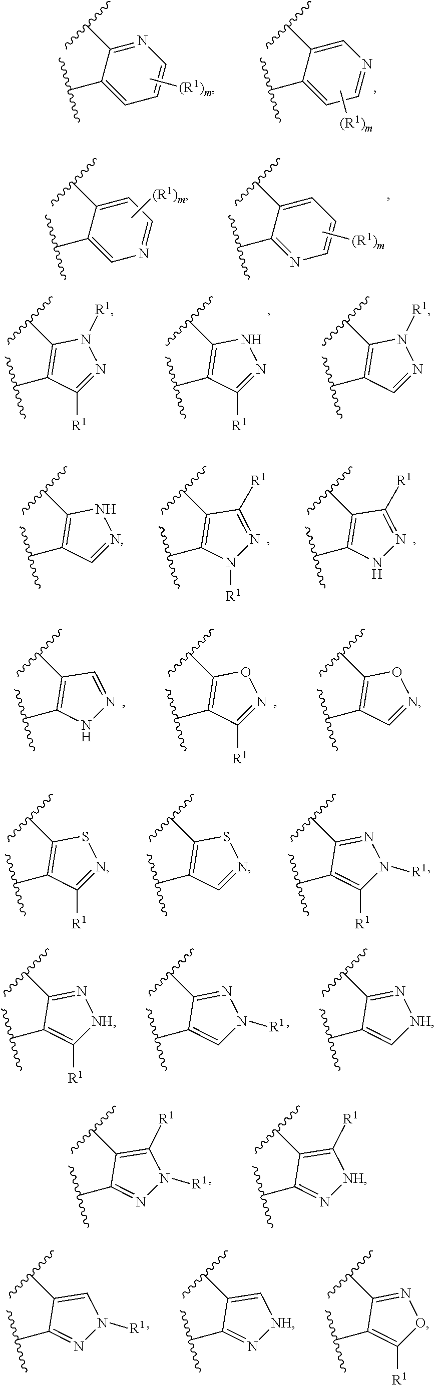

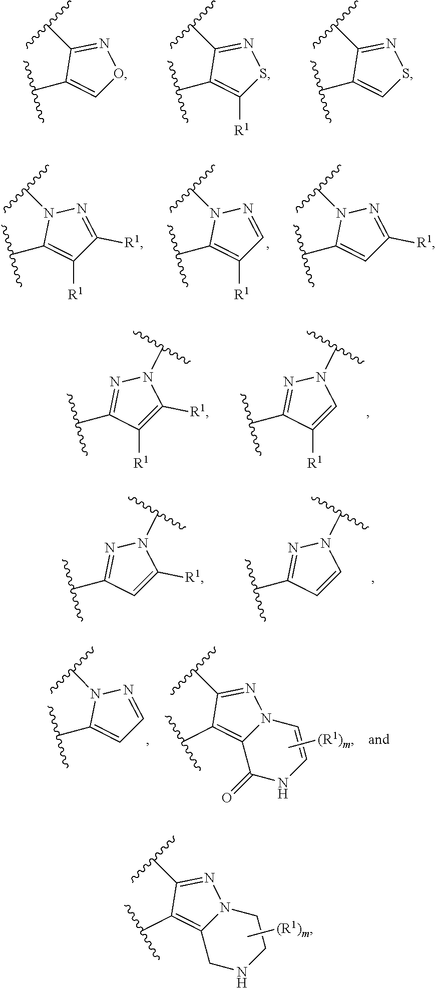

wherein each “ [symbol] ” represent a point of covalent attachment.

Tn some embodiments, ring A is a 5- or 6-membered heteroarylene selected from the group consisting of

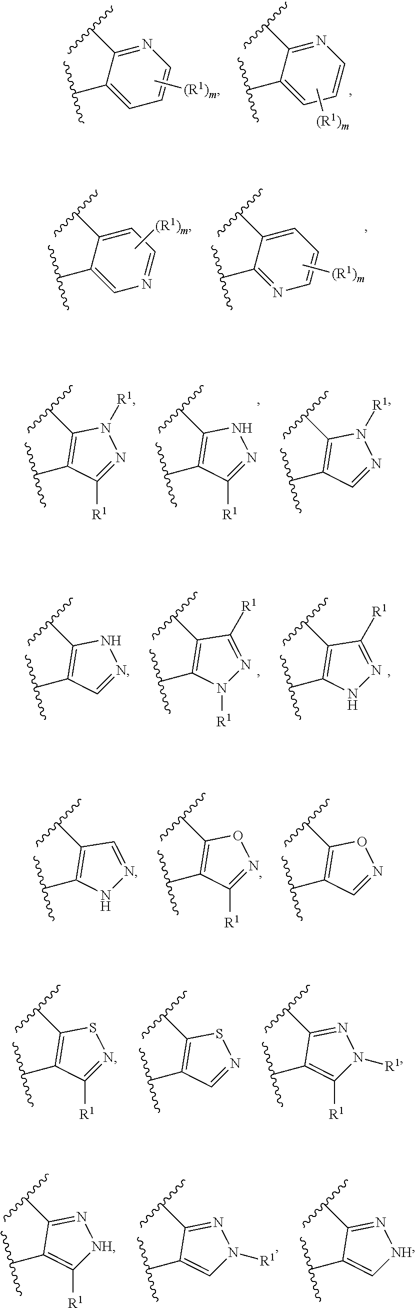

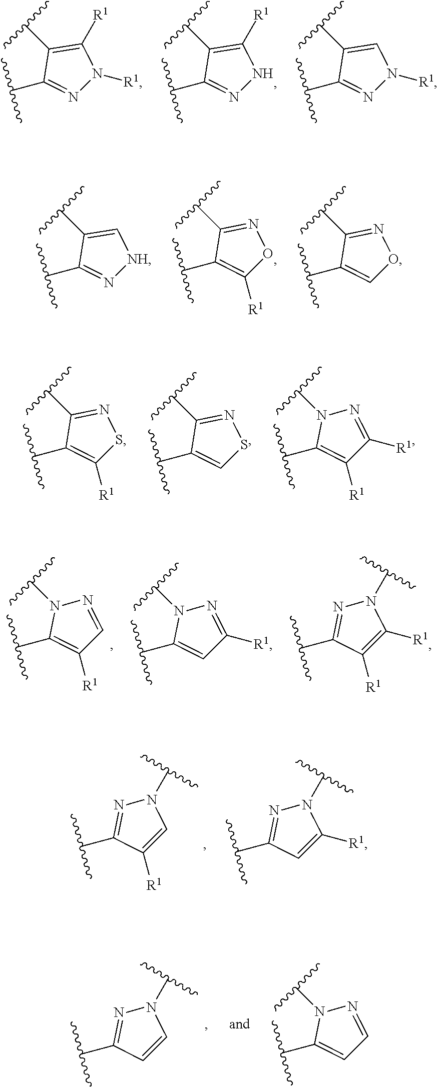

wherein each “ [symbol] ” represents a point of covalent attachment, and each R¹ is independently as described herein.

In some embodiments, ring A is a 5- to 10-membered heteroarylene selected from the group consisting of

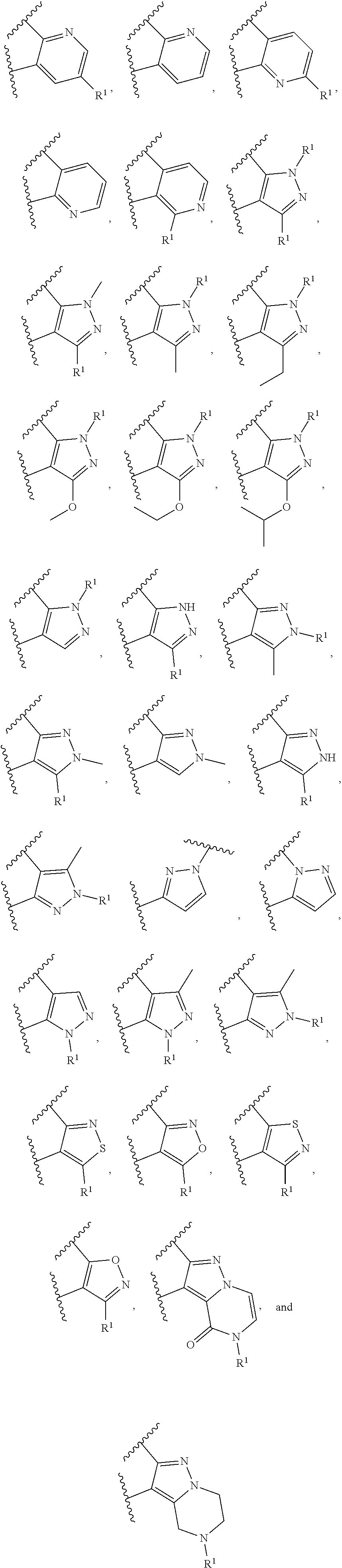

wherein each “ [symbol] ” represents a point of covalent attachment.

In some embodiments, ring A is a 5- or 6-membered heteroarylene selected from the group consisting of

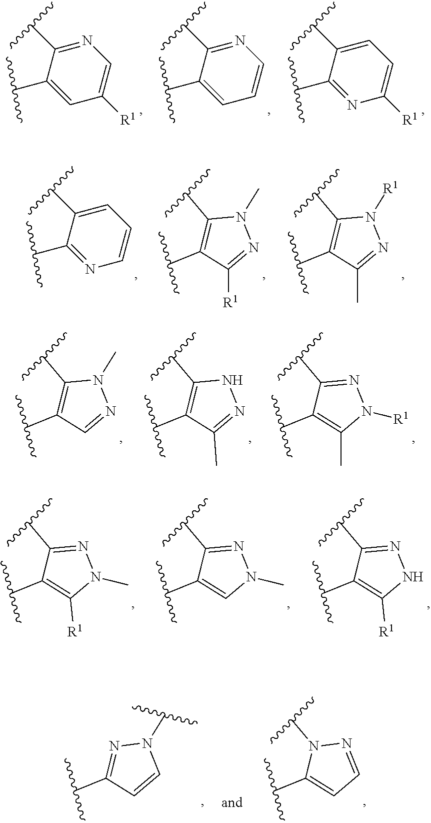

wherein each “ [symbol] ” represents a point of covalent attachment, and each R¹ is as described herein.

In some embodiments, each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{e}R^{f}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, each R¹ is independently deuterium, halogen, C₁-C₆ alkyl, C₃-C₆ cycloalkyl, 3- to 7-membered heterocycloalkyl, —CN, —OR^{a}, or —C(O)NR^{a}R^{b}, wherein each hydrogen atom in C₁-C₆ alkyl, C₃-C₆ cycloalkyl, 3- to 7-membered heterocycloalkyl, is independently optionally substituted deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R_{d}, —NR^{c}C(O)R^{d}, —N(CR^{c})(CC(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂R^{c}, —CN, or —NO₂.

In some embodiments, each R¹ is independently deuterium, halogen, C₁-C₆ alkyl, C₃-C₆ cycloalkyl, 3- to 7-membered heterocycloalkyl, or —C(O)NR^{a}R^{b}, wherein each hydrogen atom in C₁-C₆ alkyl, C₃-C₆ cycloalkyl, 3- to 7-membered heterocycloalkyl, is independently optionally substituted deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{e}, —CN, or —NO₂.

In some embodiments, two R¹ taken together with the atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{c}S(O)R^{f}, —NR^{c}S(O)₂R^{f}, —NR^{c}S(O)NR^{e}R^{f}, —NR^{e}S(O)₂NR^{e}R^{f}, —C(O)R^{c}, —C(O)OR^{c}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring A is a 5- to 10-membered heteroarylene selected from the group consisting of

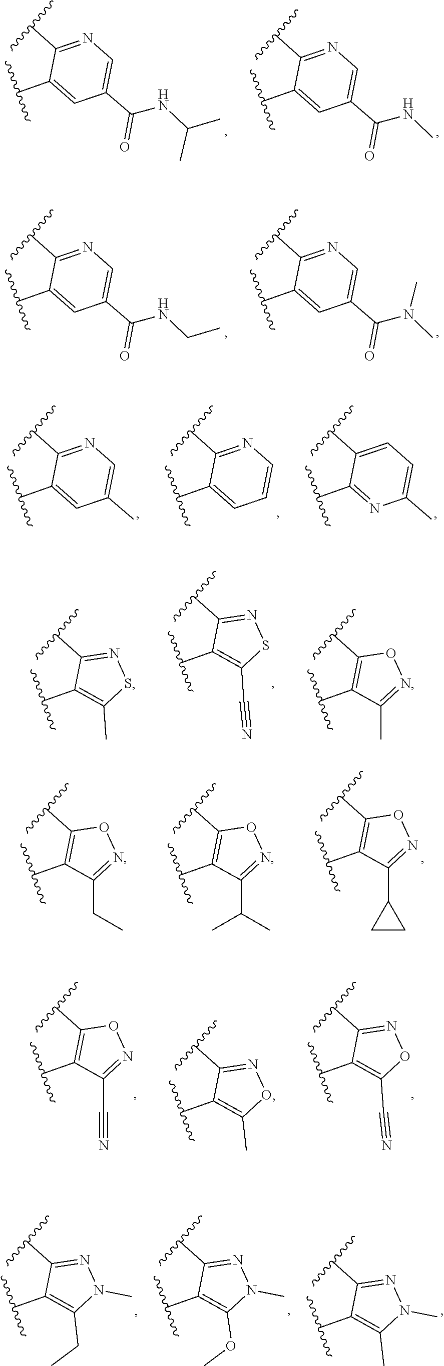

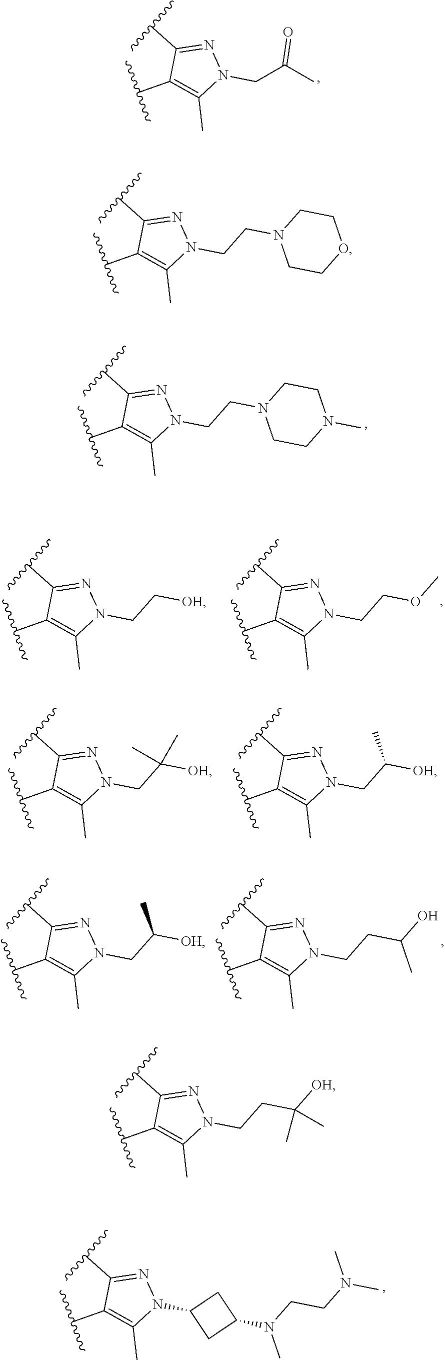

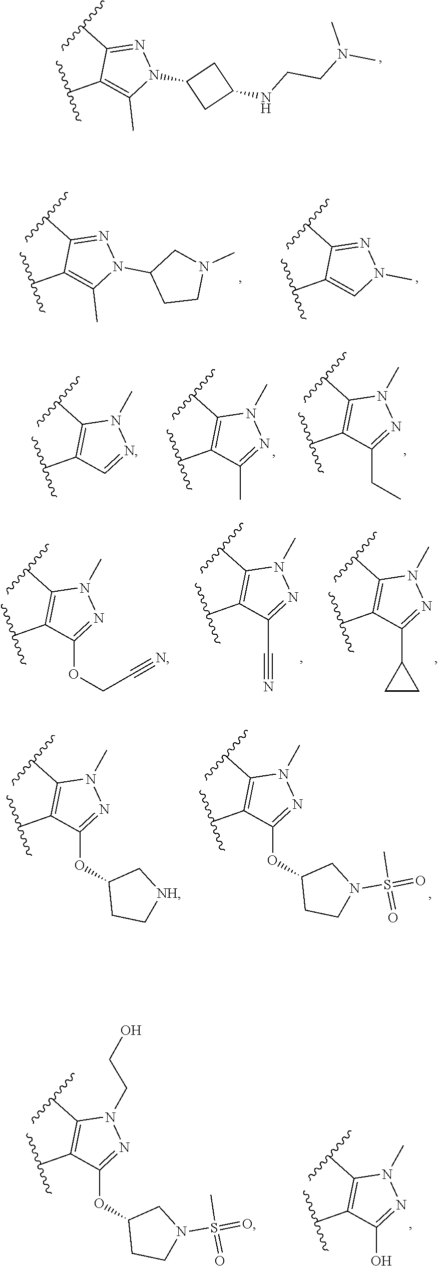

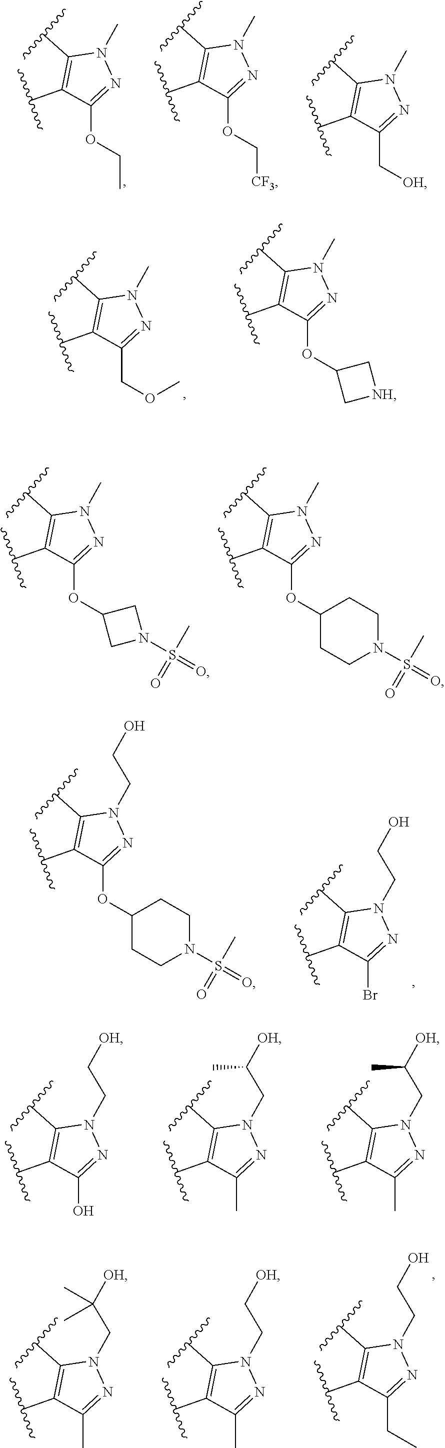

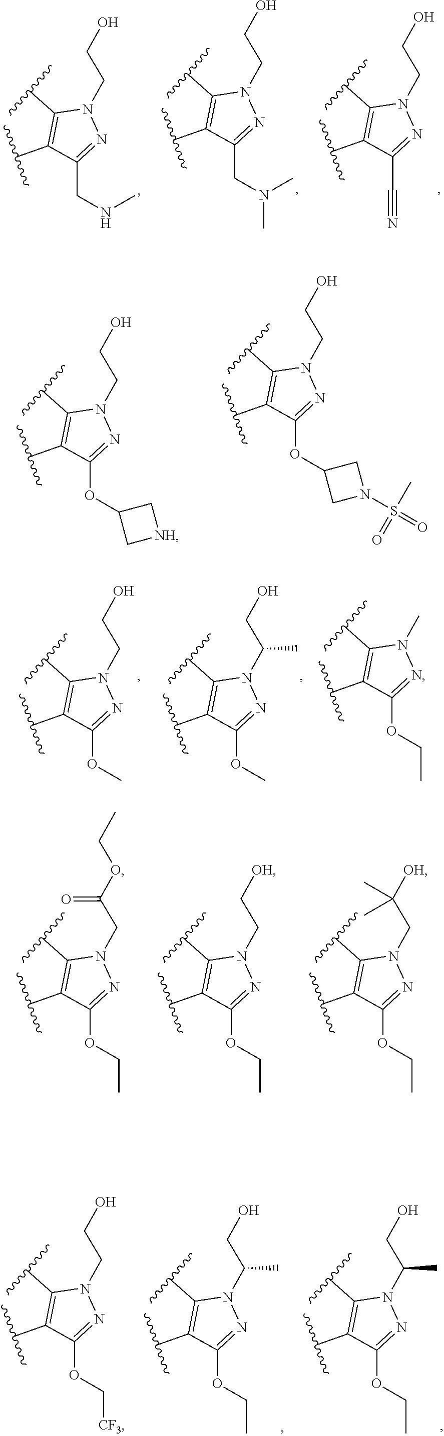

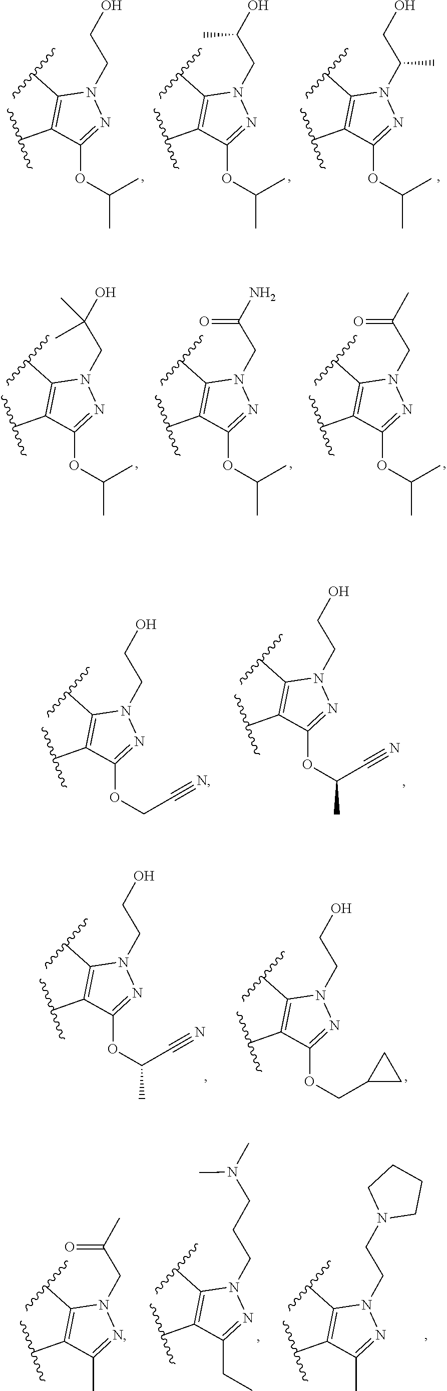

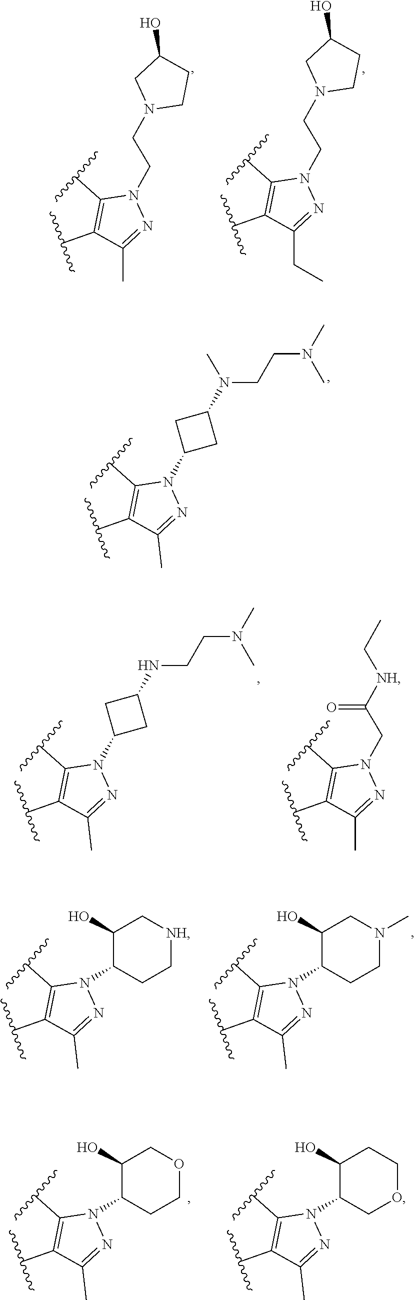

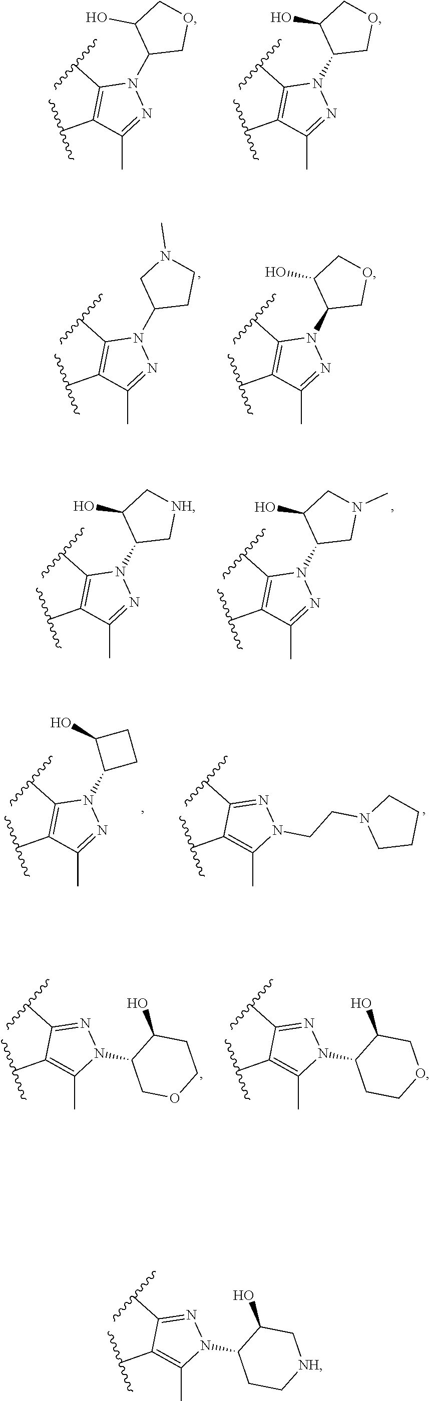

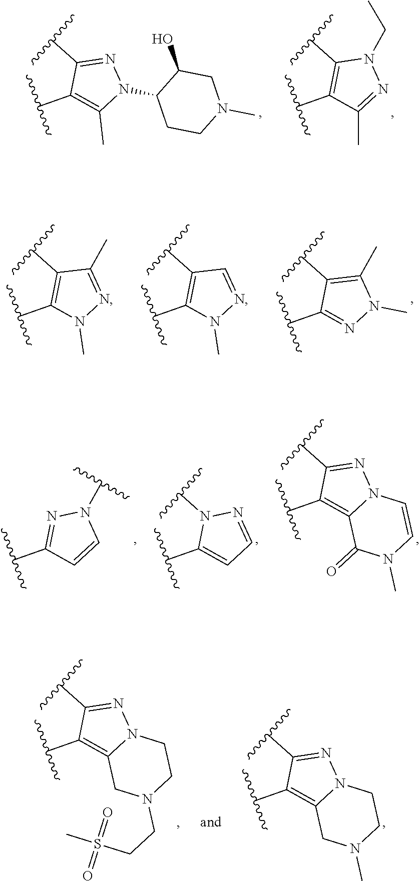

wherein each “ [symbol] ” represents a print of covalent attachment.

In some embodiments, Ring A is a 5- or 6-membered heteroarylene selected from the group consisting of

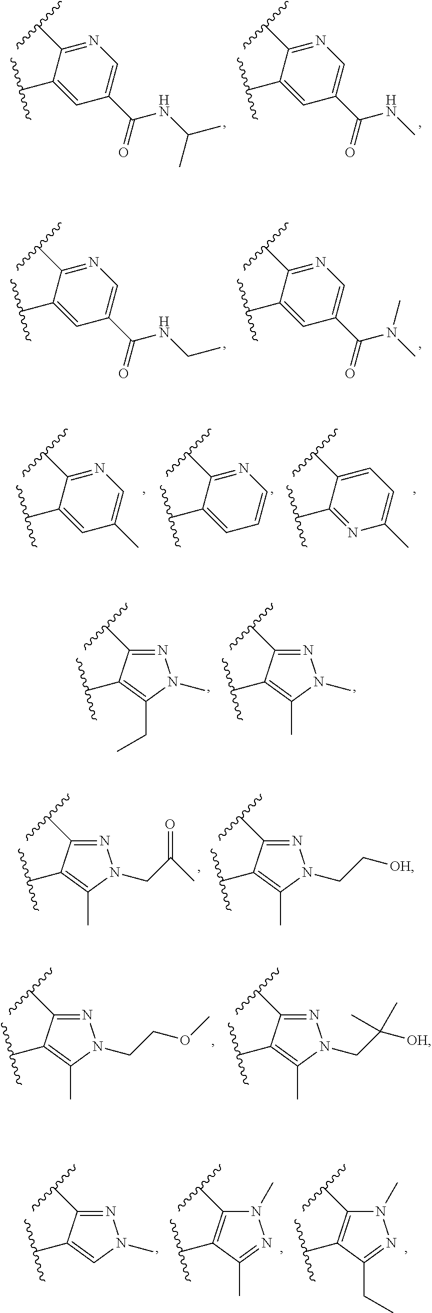

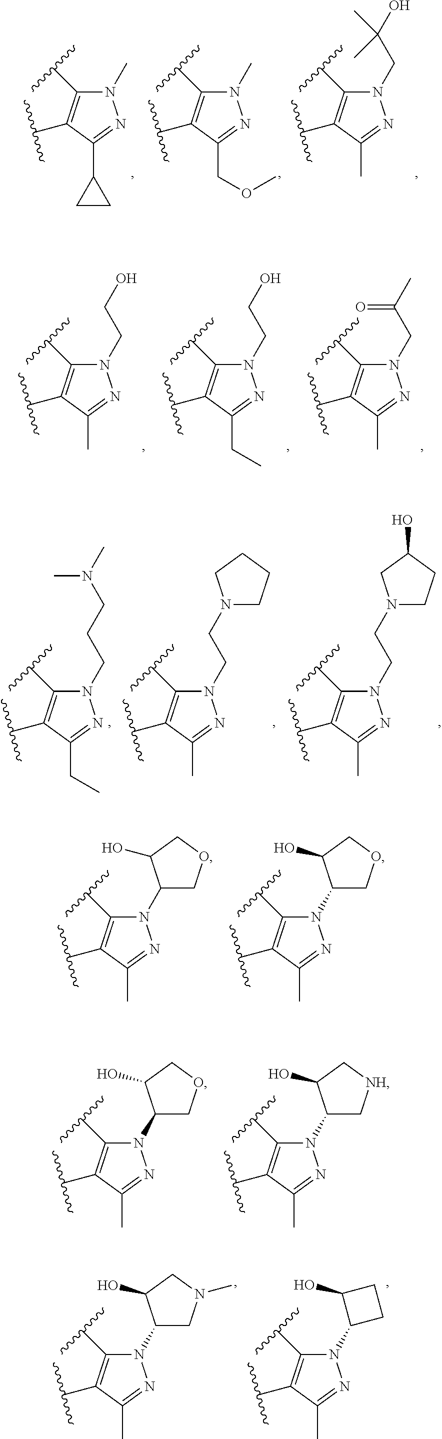

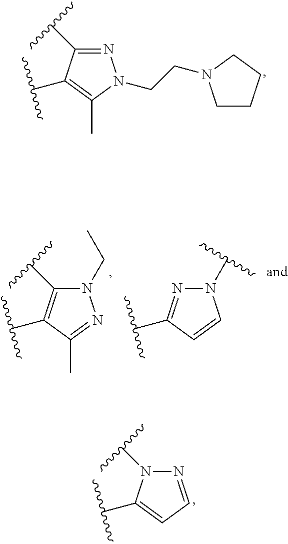

wherein each “ [symbol] ” represents a point of covalent attachment.

In some embodiments, Ring A is mono- or bi-cyclic C₆-C₁₀ arylene. In some embodiments, Ring A is monocyclic C₆-C₁₀ arylene. In some embodiments, Ring A is bicyclic C₆-C₁₀ arylene.

In some embodiments, Ring A is a C₆-C₁₀ mono- or bi-cyclic arylene, wherein each hydrogen atom in C₆-C₁₀ mono- or bi-cyclic arylene is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, Ring A is phenylene or naphthylene, wherein each hydrogen atom in phenylene or naphthylene is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)RC, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, Ring A is a C₆-C₁₀ mono- or bi-cyclic arylene, wherein each is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, and —NO₂.

In some embodiments, Ring A is phenylene or naphthylene, wherein each is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is a C₆-C₁₀ arylene, and m is as defined herein. In some embodiments, ring A is a phenylene, and m is as defined herein.

In some embodiments, m is 0, 1, 2, 3, or 4. In some embodiments, m is 0, 1, 2, or 3. In some embodiments, m is 0, 1, or 2. In some embodiments, m is 0 or 1. In some embodiments, m is 0. In some embodiments, m is 1. In some embodiments, m is 2. In some embodiments, m is 3. In some embodiments, m is 4.

In some embodiments, each R¹ is independently methyl, ethyl, F, Cl, Br, —CN,

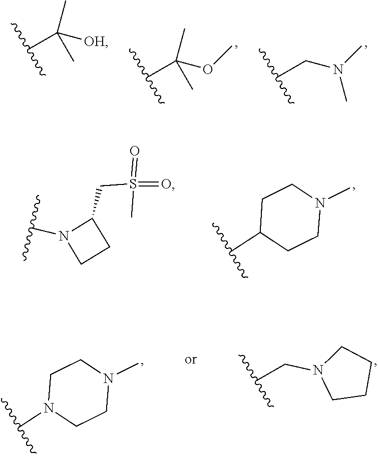

wherein each “ [symbol] ” represents a point of covalent attachment. In some embodiments, R¹ is methyl, ethyl, F, Cl, Br, or

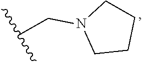 [SMILES: *CN1CCCC1]

wherein “ [symbol] ” represents a point of covalent attachment. In some embodiments, ring A is a phenylene, n is 1, and R¹ is methyl, ethyl, F, Cl, Br, or

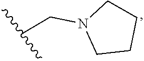 [SMILES: *CN1CCCC1]

wherein “ [symbol] ” represents a point of covalent attachment.

In some embodiments, ring A is a phenylene, m is 1, and R¹ is methyl, ethyl, F, Cl, Br, —CN,

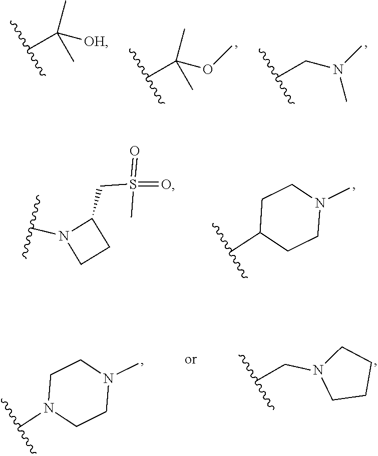

wherein each “ [symbol] ” represents a point of covalent attachment.

In some embodiments, ring A is of the formula

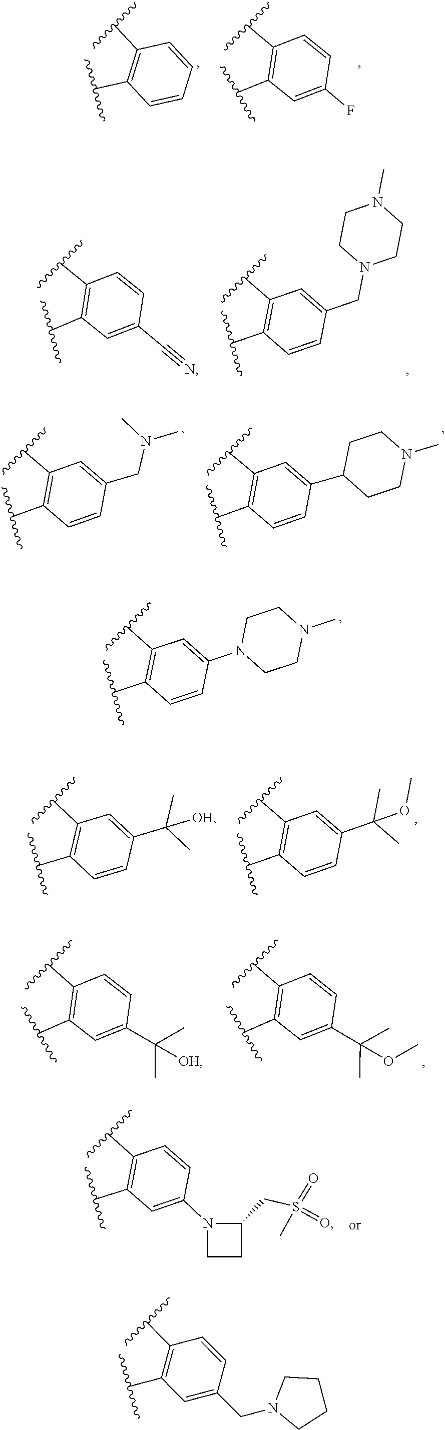

wherein each “ [symbol] ” represents a point of covalent attachment. In some embodiments, ring A is ring A is of the formula

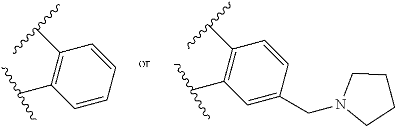 [SMILES: *c1ccccc1* ; *c1ccc(CN2CCCC2)cc1*]

wherein each “ [symbol] ” represents a point of covalent attachment.

In some embodiments, ring B is a 5- membered heteroarylene.

In some embodiments, ring B is pyrrolylene, isoxazolylene, isothiazolylene, pyrazolylene, or imidazolylene, wherein each hydrogen atom in pyrrolylene, isoxazolylene, isothiazolylene, pyrazolylene, and imidazolylene, is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)RC, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is pyrrolylene, isoxazolylene, isothiazolylene, pyrazolylene, or imidazolylene, wherein each is optionally substituted with 1, 2, 3, 4, or 5 of R² (n of R²), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, and —NO₂.

In some embodiments, n is 0, 1, 2, 3, or 4. In some embodiments, n is 0, 1, 2, or 3. In some embodiments, n is 0, 1, or 2. In some embodiments, n is 0 or 1. In some embodiments, n is 0. In some embodiments, n is 1. In some embodiments, n is 2. In some embodiments, n is 3.

In some embodiments, n is 4.

In some embodiments, ring B is a 5-membered heteroarylene selected from the group consisting of

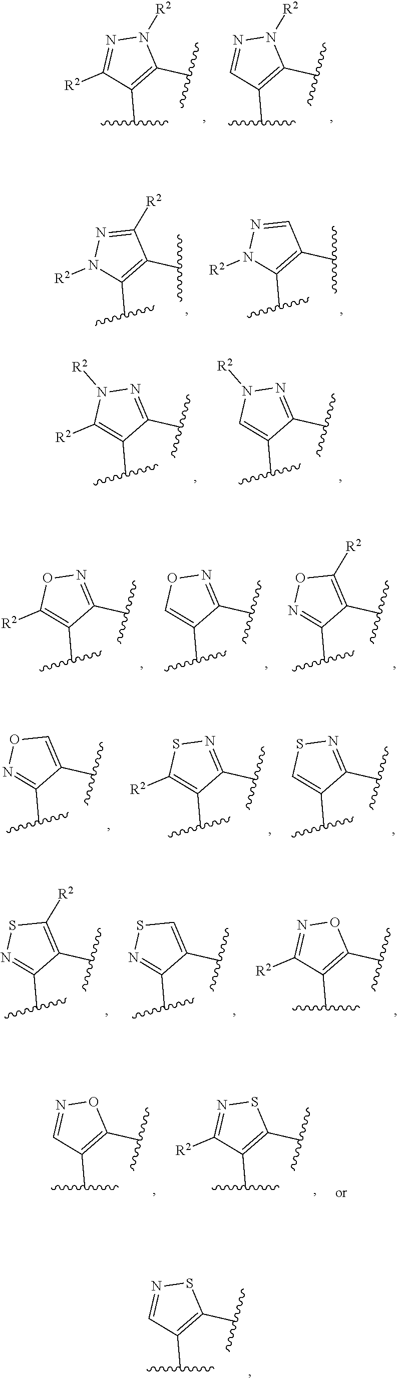

wherein each “ [symbol] ” represents a point of covalent attachment.

In some embodiments, ring B is a 5-membered heteroarylene selected from the group consisting of

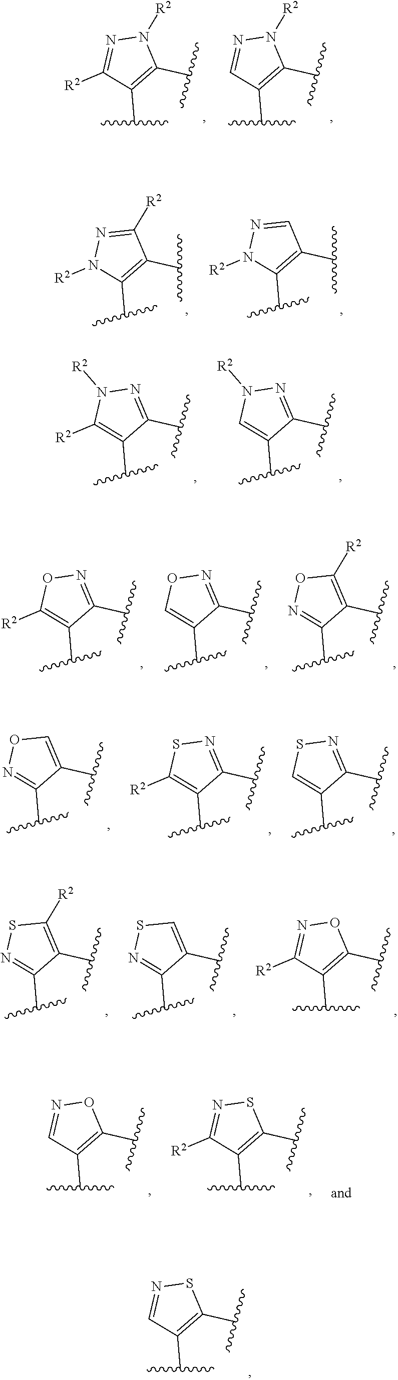

wherein each “ [symbol] ” represents a point of covalent attachment, and each R² is independently as described herein.

In some embodiments, ring B is a 5-membered heteroarylene selected from the group consisting of

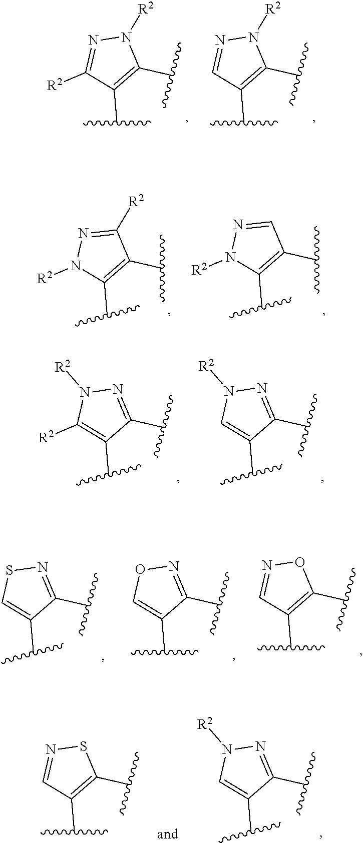

wherein each “ [symbol] ” represents a point of covalent attachment.

In some embodiments, ring B is a 5-membered heteroarylene selected from the group consisting of

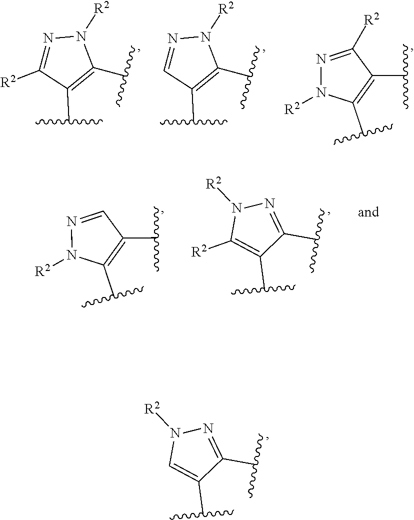

wherein each “ [symbol] ” represents a point of covalent attachment, and each R² is independently as described herein.

In some embodiments, each R² is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR., —OC(O)Rc, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, each R² is independently deuterium or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{c}R^{d}, —OC(═N)NR^{c}R^{d}, —OS(O)R^{e}, —OS(O)₂R^{c}, —OS(O)NR^{c}R^{d}, —OS(O)₂NR^{c}R^{d}, —SR^{c}, —S(O)RC, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{e}, —CN, or —NO₂. In some embodiments two R² taken together with the atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, ring B is a 5-membered heteroarylene selected from the group consisting of

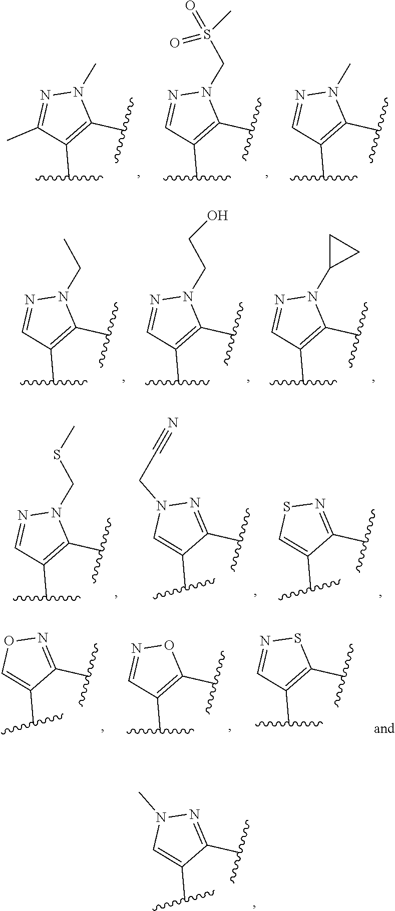

wherein each “ [symbol] ” represents a point of covalent attachment.

In some embodiments, q is 0, 1, or 2. In some embodiments, q is 0 or 1. In some embodiments, q is 0. In some embodiments, q is 1.

In some embodiments, R³ is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R³ is halogen. In some embodiments, R³ is fluoro, chloro, or bromo. In some embodiments, two R³ taken together with the atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R¹ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{c}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R⁴ is H, deuterium, C₁-C₆ alkyl, —C(O)R^{c}, —C(O)OR^{e}, —C(O)NR^{c}R^{d}, —P(O)₂R^{c}R^{d}, —P(O)₂NR^{c}R^{d}, —P(O)₂OR^{e}, or —S(O)₂OR^{c}. In some embodiments, R⁴ is H or deuterium. In some embodiments, R⁴ is H. In some embodiments, R⁴ is C₁-C₆ alkyl.

In some embodiments, R⁴ is methyl or ethyl.

In some embodiments, each R⁵, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{e}, —OS(O)₂R^{e}, —OS(O)NR^{c}R^{d}, —OS(O)₂NR^{c}R^{d}, —SR^{c}, —S(O)R^{e}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{e}, —C(O)OR^{e}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, each R⁵ is independently H, methyl, ethyl, —C(O)CH₃, or —C(O)CH₂CH₃; or an R⁵ combines with an R⁶ to form a 3- to 7-membered heterocycloalkyl.

In some embodiments, each R⁵, when present, is independently H, deuterium, C₁-C₆ alkyl, C₂-C₆ alkenyl, C₂-C₆ alkynyl, C₃-C₆ cycloalkyl, —C(O)R^{a}, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, each R⁵ is independently H, methyl, ethyl, —C(O)CH₃, or —C(O)CH₂CH₃; or an R⁵ combines with an R⁶ to form a 3- to 7-membered heterocycloalkyl.

In some embodiments, each L is independently —C(R⁶)(R⁷)—, —C(O)—, —O—, or —N(R⁵)—, provided that (L)ₚ does not comprise a —O—O— or a —O—N(R⁵)— bond, and the point of covalent attachment of (L)ₚ to ring A does not form a —N—N— or a —O—N— bond.

In some embodiments, p is 3, 4, 5, 6, 7, 8, or 9. In some embodiments, p is 4, 5, 6, 7, 8, or 9. In some embodiments, p is 4, 5, 6, 7, or 8. In some embodiments, p is 4, 5, 6, or 7. In some embodiments, p is 5, 6, 7, or 8. In some embodiments, p is 5, 6, or 7. In some embodiments, p is 3, 4, 5, 6, or 7. In some embodiments, p is 3, 4, 5, or 6. In some embodiments, p is 3. In some embodiments, p is 4. In some embodiments, p is 5. In some embodiments, p is 6. In some embodiments, p is 7. In some embodiments, p is 8. In some embodiments, p is 9.

In some embodiments, p1 is 2, 3, or 4. In some embodiments, p1 is 2. In some embodiments, p1 is 3. In some embodiments, p1 is 4.

In some embodiments, -(L)ₚ- comprises —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂O(CR⁶R⁷)₂—, —O(CR⁶R⁷)₂O(CR⁶R⁷)₂O—, —CR⁶R⁷O—CR⁶R⁷—C(O)N(R⁵)—(CR⁶R⁷)₂—, —(CR⁶R⁷)₃O(CR⁶R⁷)₂—, —(CR⁶R⁷)₂O(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

In some embodiments, -(L)ₚ- is —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂O(CR⁶R⁷)₂—, —O(CR⁶R⁷)₂O(CR⁶R⁷)₂O—, —CR⁶R⁷O—CR⁶R⁷—C(O)N(R⁵)—(CR⁶R⁷)₂—, —(CR⁶R⁷)₃O(CR⁶R⁷)₂—, —(CR⁶R⁷)₂O(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

In some embodiments, -(L)ₚ- comprises —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂O(CR⁶R⁷)₂O—, —CR⁶R⁷O—CR⁶R⁷—C(O)N(R⁵)—(CR⁶R⁷)₂—, —(CR⁶R⁷)₃O(CR⁶R⁷)₂—, —(CR⁶R⁷)₂O(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O—CR⁶R⁷—, —O(CR⁶R⁷)₂O(CR⁶R⁷)₂—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

In some embodiments, -(L)ₚ- is —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂O(CR⁶R⁷)₂O—, —CR⁶R⁷O—CR⁶R⁷—C(O)N(R⁵)—(CR⁶R⁷)₂—, —(CR⁶R⁷)₃O(CR⁶R⁷)₂—, —(CR⁶R⁷)₂O(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O—CR⁶R⁷—, —O(CR⁶R⁷)₂O(CR⁶R⁷)₂—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

In some embodiments, each R⁵ is H, methyl, ethyl, —C(O)CH₃, or —C(O)CH₂CH₃; or an R⁵ combines with an R⁶ to form a 3- to 7-membered heterocycloalkyl. In some embodiments, each R⁶ is H or C₁-C₆ alkyl; or an R⁶ combines with an R⁵ to form a 3- to 7-membered heterocycloalkyl. In some embodiments, one R⁶ is methyl, and the remaining R⁶ and R⁷ are H.

In some embodiments, each R⁵ is independently H, methyl, ethyl, —C(O)CH₃, or —C(O)CH₂CH₃; or R⁵, when present, and an R⁶ or R⁷, when present, taken together with the atom or atoms to which they are attached, combine to form a 3- to 7-membered heterocycloalkyl; wherein each hydrogen atom in the 3- to 7-membered heterocycloalkyl formed when R⁵ and an R⁶ or R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, an R⁶ and R⁷, when present, taken together with the atom or atoms to which they are attached, combine to form a 3- to 7-membered heterocycloalkyl; wherein each hydrogen atom in the 3- to 7-membered heterocycloalkyl formed when R⁶ and R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{c}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, -(L)ₚ- comprises —O—(CH₂)₂O—CH₂—, —OC(H)(CH₃)—CH₂—O—CH₂—, —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —OCH₂—C(H)(CH₃)—N(CH(CH₃)₂)CH₂—, —OCH₂—C(H)(CH₂F)—N(CH₃)CH₂—, —OCH₂—C(H)(CH₂CH₃)—N(CH₃)CH₂—, —O(CH₂)₂N(C(O)CH₃)CH₂—, —OC(H)(CH₃)CH₂N(H)CH₂—, —OC(H)(CH₃)CH₂N(CH₃)CH₂—, —OC(H)(CH₂CN)CH₂N(CH₃)CH₂—, —OC(H)(CH₂CH₃)CH₂N(CH₃)CH₂—, —OC(H)(CH₂F)CH₂N(CH₃)CH₂—, —OC(H)(CHF₂)CH₂N(CH₃)CH₂—, —OC(H)(CH₂OH)CH₂N(CH₃)CH₂—, —OC(H)(CH₃)C(O)N(CH₃)CH₂—, —OC(H)(cyclobutyl)CH₂N(CH₃)CH₂—, —OC(H)(CH₃)CH₂N(CH₂CH₃)CH₂—, —OC(H)(CH₃)CH₂N(CH(CH₃)₂)—CH₂—, —OC(H)(CH₃)CH₂N(CH₃)—C(H)(CH₃)—, —OC(H)(CH₃)CH₂N(CH(oxetan-3-yl)-CH₂—, —OC(H)(CH₃)CH₂N(cyclopropyl)-CH₂—, —OCH₂C(H)(CH₃)N(cyclopropyl)-CH₂—, —OC(H)(CH₃)CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—C(H)(CH₃)—CH₂O—, —CH₂N(CH₃)—(CH₂)₂(C(H)CH₃)CH₂O—, —CH₂N(CH₃)—(CH₂)—(C(H)CH₃)O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—C(H)(CH₃)—O—, —O(CH₂)₂C(H)(CH₃)CH₂O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —OC(H)(CH₃)CH₂OCH₂—, —O(CH₂)₂OCH₂C(H)(CH₃)O—, —O(CH₂)₂₀C(H)(CH₃)—CH₂O—,

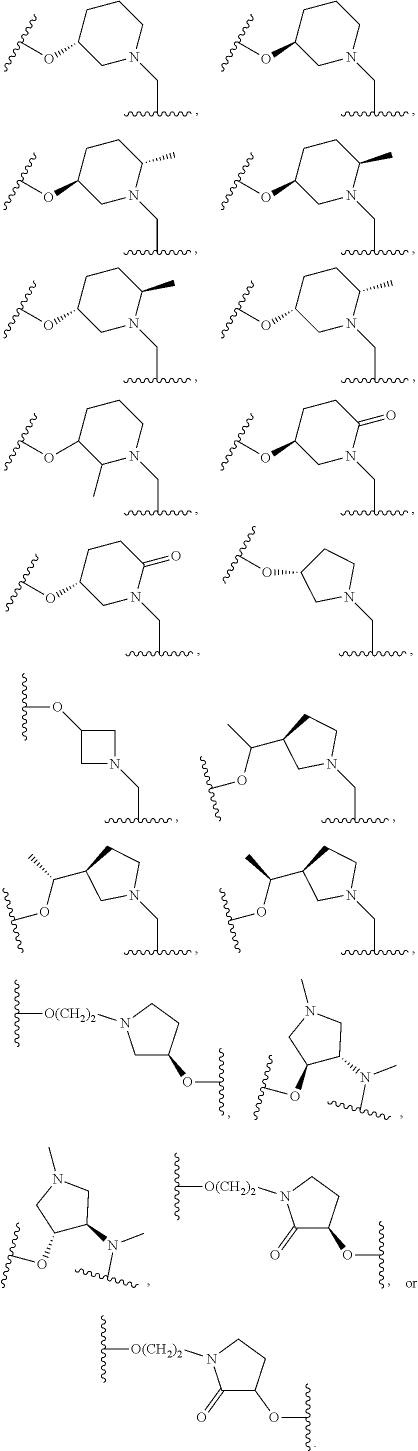

In some embodiments, -(L)ₚ- is —O—(CH₂)₂O—CH₂—, —OC(H)(CH₃)—CH₂—O—CH₂—, —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —OCH₂—C(H)(CH₃)—N(CH(CH₃)₂)CH₂—, —OCH₂—C(H)(CH₂F)—N(CH₃)CH₂—, —OCH₂—C(H)(CH₂CH₃)—N(CH₃)CH₂—, —O(CH₂)₂N(C(O)CH₃)CH₂—, —OC(H)(CH₃)CH₂N(H)CH₂—, —OC(H)(CH₃)CH₂N(CH₃)CH₂—, —OC(H)(CH₂CN)CH₂N(CH₃)CH₂—, —OC(H)(CH₂CH₃)CH₂N(CH₃)CH₂—, —OC(H)(CH₂F)CH₂N(CH₃)CH₂—, —OC(H)(CHF₂)CH₂N(CH₃)CH₂—, —OC(H)(CH₂OH)CH₂N(CH₃)CH₂—, —OC(H)(CH₃)C(O)N(CH₃)CH₂—, —OC(H)(cyclobutyl)CH₂N(CH₃)CH₂—, —OC(H)(CH₃)CH₂N(CH₂CH₃)CH₂—, —OC(H)(CH₃)CH₂N(CH(CH₃)₂)—CH₂—, —OC(H)(CH₃)CH₂N(CH₃)—C(H)(CH₃)—, —OC(H)(CH₃)CH₂N(CH(oxetan-3-yl)-CH₂—, —OC(H)(CH₃)CH₂N(cyclopropyl)-CH₂—, —OCH₂C(H)(CH₃)N(cyclopropyl)-CH₂—, —OC(H)(CH₃)CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—C(H)(CH₃)—CH₂O—, —CH₂N(CH₃)—(CH₂)₂(C(H)CH₃)CH₂O—, —CH₂N(CH₃)—(CH₂)—(C(H)CH₃)O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—C(H)(CH₃)—O—, —O(CH₂)₂C(H)(CH₃)CH₂O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —OC(H)(CH₃)CH₂OCH₂—, —O(CH₂)₂OCH₂C(H)(CH₃)O—, —O(CH₂)₂₀C(H)(CH₃)—CH₂O—,

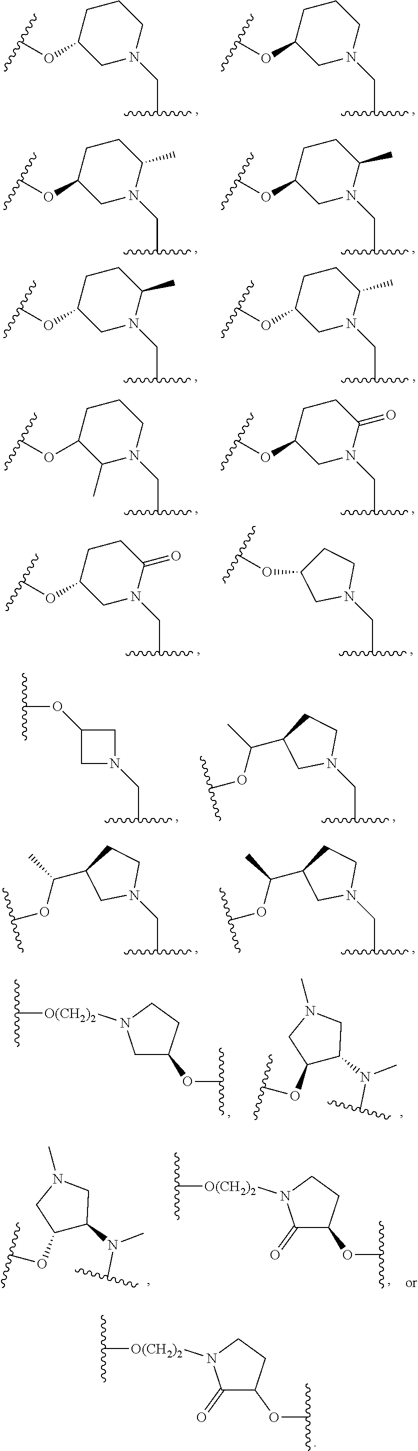

In some embodiments, -(L)ₚ- comprises —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —O(CH₂)₂N(C(O)CH₃)CH₂—, —O(C(H)(CH₃))CH₂N(H)CH₂—, —O(C(H)(CH₃))CH₂N(CH₃)CH₂—, —O(C(H)(CH₃))CH₂N(CH₂CH₃)CH₂—, —O(C(H)(CH₃))CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—(CH₂)₂(C(H)CH₃)CH₂O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—(C(H)(CH₃)—O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —O(CH₂)₂OCH₂C(H)(CH₃)O—, —O(CH₂)₂₀C(H)(CH₃)—CH₂O—, or —O(CH₂)₂ N

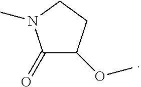

In some embodiments, -(L)ₚ- is —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —O(CH₂)₂N(C(O)CH₃)CH₂—, —O(C(H)(CH₃))CH₂N(H)CH₂—, —O(C(H)(CH₃))CH₂N(CH₃)CH₂—, —O(C(H)(CH₃))CH₂N(CH₂CH₃)CH₂—, —O(C(H)(CH₃))CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—(CH₂)₂(C(H)CH₃)CH₂O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—(C(H)(CH₃)—O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —O(CH₂)₂OCH₂C(H)(CH₃)O—, —O(CH₂)₂₀C(H)(CH₃)—CH₂O—, and —O(CH₂)₂

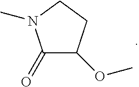

In connection with any of the embodiments described herein, (L)ₚ does not comprise a —NR⁵C(O)— directly covalently attached to ring A. In connection with any of the embodiments described herein, (L)ₚ does not comprise a —O—CR⁶R⁷—fragment directly covalently to ring A.

In connection with any of the embodiments described herein, (L)ₚ does not comprise a —NR⁵C(O)— directly covalently attached to ring A, and (L)ₚ does not comprise a —O—CR⁶R⁷-fragment directly covalently to ring A.

In some embodiments, the disclosure provides a compound selected from the group consisting of (11E)-1-methyl-1,18,19,21-tetrahydro-8H-10,7,4-(ethan[1]yl[1 ,2]diylidene)pyrazolo[3,4-f][1,4,12,13]benzodioxadiazacyclooctadecine;
(11E)-1-[(methanesulfonyl)methyl]-19,20-dihydro-1H,8H,18H-10,7,4-(ethan[1]yl[1,2]diylidene)pyrazolo[3,4-f][1,5,12,13]benzodioxadiazacyclooctadecine;
(11E)-1-methyl-18,19,20,21-tetrahydro-1H,8H-10,7,4-(ethan[1]yl[1,2]diylidene)pyrazolo[3,4-g][1,6,13,14]benzodioxadiazacyclononadecine;
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-[(4-methylpiperazin-1-yl)methyl]-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-[(dimethylamino)methyl]-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(1-methylpiperidin-4-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-(2-methoxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(4-methylpiperazin-1-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-fluoro-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-(2-hydroxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-{(2S)-2-[(methanesulfonyl)methyl]azetidin-1-yl}-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-25-oxo-3,5,6,9,10,19-hexahydro-8H-20,22-etheno-7,10-methanodipyrazolo[3,4-i:4′,3′-m][1,8,5]benzodioxazacyclooctadecine-14-carbonitrile; and
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecine;
or a pharmaceutically acceptable salt thereof.

32. The compound of clause 1 or 2, selected from the group consisting of (18E)-8-methyl-N-(propan-2-yl)-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N,8-dimethyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N,N,8-trimethyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N-ethyl-8-methyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(10S,18E)-8,10,16-trimethyl-2,8,11,12-tetrahydro-10H-3,5-ethenodipyrazolo[3′,4′:10,11;4″,3″:14,15][1,5]dioxacyclopentadecino[7,6-b]pyridine;
(10S,18E)-17-ethoxy-8,10,12-trimethyl-2,8,10,11,12,13-hexahydro-3,5-ethenodipyrazolo[3,4-j:4′,3′-n]pyrido[4,3-f][1,4]oxazacyclopentadecine; and
(10S,18E)-16-ethoxy-8,10,12-trimethyl-2,8,10,11,12,13-hexahydro-3,5-ethenodipyrazolo[3,4-j:4′,3′-n]pyrido[3,2-f][1,4]oxazacyclopentadecine;
or a pharmaceutically acceptable salt thereof.
33. The compound of clause 1 or 2, selected from the group consisting of (17E)-8,14,16-trimethyl-2,8,9,11,12,14-hexahydro-3,5-etheno[1,4]dioxacyclopentadecino[11,10-c:15,14-c′:6,7-c″ ]tripyrazole;
2-[(17E)-8,9,16-trimethyl-8,9,11,12-tetrahydro-3,5-etheno[1,4]dioxacyclopentadecino[11,10-c:15,14-c′:6,7-c″ ]tripyrazol-14(2H)-yl]ethan-1-ol;
(17E)-8,14,16-trimethyl-2,11,12,14-tetrahydro-8H,10H-3,5-etheno[1,5]dioxacyclopentadecino[10,11-c:15,14-c′:6,7-c″ ]tripyrazole;
(17E)-19-fluoro-8,14,16-trimethyl-2,11,12,14-tetrahydro-8H,10H-3,5-etheno[1,5]dioxacyclopentadecino[10,11-c:15,14-c′:6,7-c″]tripyrazole;
(17E)-16-ethyl-8,14-dimethyl-2,11,12,14-tetrahydro-8H,10H-3,5-etheno[1,5]dioxacyclopentadecino[10,11-c:15,14-c′:6,7-c″ ]tripyrazole;
1-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-2-methylpropan-2-ol;
1-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]propan-2-one;
2-[(17E)-8,16-dimethyl-2,8,11,12-tetrahydro-10H,14H-3,5-etheno[1,5]dioxacyclopentadecinol 10,11-c:15,14-c′:6,7-c″|tripyrazol-14-yl|ethan-1-ol;
(17E)-8,16-dimethyl-14-[2-(pyrrolidin-1-yl)ethyl]-2,11,12,14-tetrahydro-8H,10H-3,5-etheno[1,5]dioxacyclopentadecino[10,11-c:15,14-c′:6,7-c″ ]tripyrazole;
(3S)-1-{2-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]ethyl}pyrrolidin-3-ol;
(17E)-16-ethyl-8,12,14-trimethyl-2,11,12,14-tetrahydro-8H,10H-3,5-etheno[1,5]dioxacyclopentadecino[10,11-c:15,14-c′:6,7-c″ ]tripyrazole;
(10S,17E)-8,10,14,16-tetramethyl-2,11,12,14-tetrahydro-8H,10H-3,5-etheno[1,5]dioxacyclopentadecino[10,11-c:15,14-c′:6,7-c″ ]tripyrazole;
(10R,17E)-8,10,14,16-tetramethyl-2,11,12,14-tetrahydro-8H,10H-3,5-etheno[1,5]dioxacyclopentadecino[10,11-c:15,14-c′:6,7-c″ ]tripyrazole;
(12S,17E)-16-ethyl-8,12,14-trimethyl-2,11,12,14-tetrahydro-8H,10H-3,5-etheno[1,5]dioxacyclopentadecino[10,11-c:15,14-c′:6,7-c″ ]tripyrazole;
(12R,17E)-16-ethyl-8,12,14-trimethyl-2,11,12,14-tetrahydro-8H,10H-3,5-etheno[1,5]dioxacyclopentadecino[10,11-c:15,14-c′:6,7-c″ ]tripyrazole;
(10S,17E)-16-ethyl-8,10,14-trimethyl-2,11,12,14-tetrahydro-8H,10H-3,5-etheno[1,5]dioxacyclopentadecino[10,11-c:15,14-c′:6,7-c″ ]tripyrazole;
(10S,17E)-16-cyclopropyl-8,10,14-trimethyl-2,11,12,14-tetrahydro-8H,10H-3,5-etheno[1,5]dioxacyclopentadecino[10,11-c:15,14-c′:6,7-c″ ]tripyrazole;
(17E)-16-(methoxymethyl)-8,14-dimethyl-2,11,12,14-tetrahydro-8H,10H-3,5-etheno[1,5]dioxacyclopentadecino[10,11-c:15,14-c′:6,7-c″ ]tripyrazole;
(17E)-8,14,16-trimethyl-2,10,11,12,13,14-hexahydro-8H-3,5-ethenotripyrazolo[3,4-f:3′,4′-j:4″,3″-n][1,4]oxazacyclopentadecine;
1-[(17E)-8,14,16-trimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one;
(17E)-8,12,14,16-tetramethyl-2,10,11,12,13,14-hexahydro-8H-3,5-ethenotripyrazolo[3,4-f:3′,4′-j:4″,3″-n][1,4]oxazacyclopentadecine;
(17E)-6,8,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(17E)-16-ethyl-8,12,14-trimethyl-2,10,11,12,13,14-hexahydro-8H-3,5-ethenotripyrazolo[3,4-f:3′,4′-j:4″,3″-n][1,4]oxazacyclopentadecine;
(17E)-7,14,16-trimethyl-2,10,11,12,13,14-hexahydro-7H-3,5-ethenotripyrazolo[3,4-f:3′,4′-j:4″,3″-n][1,4]oxazacyclopentadecine;
(17E)-7,12,14,16-tetramethyl-2,10,11,12,13,14-hexahydro-7H-3,5-ethenotripyrazolo|3,4-f:3′,4′-j:4″,3″-n∥1,4|oxazacyclopentadecine;
2-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(17E)-8,14,16-trimethyl-2,9,10,11,12,14-hexahydro-8H-3,5-ethenotripyrazolo[3,4-f:4′,3′-j:4″,3″-n][1,4]oxazacyclopentadecine;
(17E)-8,10,14,16-tetramethyl-2,9,10,11,12,14-hexahydro-8H-3,5-ethenotripyrazolo[3,4-f:4′,3′-j:4″,3″-n][1,4]oxazacyclopentadecine;
2-[(17E)-8,16-dimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-10-ethyl-8,16-dimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
(19E)-8,13,16,18-tetramethyl-2,11,12,13,14,16-hexahydro-8H,10H-3,5-etheno[1,7]dioxacycloheptadecino[12,13-c:17,16-c′:8,9-c″ ]tripyrazole;
2-[(19E)-8,13,18-trimethyl-2,8,11,12,13,14-hexahydro-10H,16H-3,5-etheno[1,7]dioxacycloheptadecino[12,13-c:17,16-c′:8,9-c″ ]tripyrazol-16-yl]ethan-t-ol;
(19E)-8,16,18-trimethyl-2,11,12,16-tetrahydro-8H,10H-3,5-ethenotripyrazolo[3,4-h:3′,4′-l:4″,3″-p][1,7,4]dioxazacycloheptadecin-13(14H)-one;
(19E)-8,12,16,18-tetramethyl-2,11,12,16-tetrahydro-8H,10H-3,5-ethenotripyrazolo[3,4-h:3′,4′-l:4″,3″-p][1,7,4]dioxazacycloheptadecin-13(14H)-one;
(13R,19E)-8,13,16,18-tetramethyl-2,10,11,13,14,16-hexahydro-8H-3,5-etheno[1,4,7]trioxacycloheptadecino[12,13-c:17,16-c′:8,9-c″ ]tripyrazole;
(13S,19E)-8,13,16,18-tetramethyl-2,10,11,13,14,16-hexahydro-8H-3,5-etheno[1,4,7]trioxacycloheptadecino[12,13-c:17,16-c′:8,9-c″ ]tripyrazole;
2-[(19E)-8,13,18-trimethyl-2,8,10,11,13,14-hexahydro-16H-3,5-etheno[1,4,7]trioxacycloheptadecino[12,13-c:17,16-c′:8,9-c″ ]tripyrazol-16-yl]ethan-1-ol;
(19E)-8,13,18-trimethyl-16-[2-(pyrrolidin-1-yl)ethyl]-2,10,11,13,14,16-hexahydro-8H-3,5-etheno[1,4,7]trioxacycloheptadecino[12,13-c:17,16-c′:8,9-c′]tripyrazole;
(3S)-1-{2-[(17E)-16-ethyl-8-methyl-2,8,11,12-tetrahydro-10H,14H-3,5-etheno[1,5]dioxacyclopentadecinol[10,11-c:15,14-c′:6,7-c″ ]tripyrazol-14-yl]ethyl}pyrrolidin-3-ol;
(19E)-22-fluoro-8,16,18-trimethyl-2,10,11,13,14,16-hexahydro-8H-3,5-etheno[1,4,7]trioxacycloheptadecino[12,13-c:17,16-c′:8,9-c′]tripyrazole;
3-|(17E)-16-ethyl-8-methyl-2,8,11,12-tetrahydro-10H,14H-3,5-etheno[1,5]dioxacyclopentadecino[10,11-c:15,14-c′:6,7-c″ ]tripyrazol-14-yl]-N,N-dimethylpropan-i-amine;
(rac)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]pyrrolidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-1-methylpyrrolidin-3-ol;
(3R,4S)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]oxolan-3-ol;
(3S,4R)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]oxolan-3-ol;
(rac)-2-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]cyclobutan-1-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]piperidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-1-methylpiperidin-3-ol;
(rac)-1,5-anhydr-3o-2,3-dideoxy-3-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-L-threo-pentitol;
(rac)-1,5-anhydro-2,4-dideoxy-2-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-L-threo-pentitol;
(12R,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(12S,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(rac)-(3S,4S)-1-methyl-4-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]pyrrolidin-3-ol;
(11S,17E)-8,11,14,16-tetramethyl-2,9,10,11,12,14-hexahydro-8H-3,5-ethenotripyrazolo[3,4-f:4′,3′-j:4″,3″-n][1,4]oxazacyclopentadecine;
(17E)-8,9,16-trimethyl-14-[2-(pyrrolidin-1-yl)ethyl]-2,8,9,11,12,14-hexahydro-3,5-etheno[1,4]dioxacyclopentadecino[11,10-c:15,14-c′:6,7-c″ ]tripyrazole;
   |(10S,17E)-8,10,14-trimethyl-2,11,12,14-tetrahydro-8H,10H-3,5-etheno[1,5]dioxacyclopentadecino[10,11-c:15,14-c′:6,7-c″]tripyrazol-16-yl]methanol;
N¹—{(1s,3s)-3-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]cyclobutyl}-N¹,N²,N²-trimethylethane-1,2-diamine;
(11S,17E)-8,10,11,14,16-pentamethyl-2,9,10,11,12,14-hexahydro-8H-3,5-ethenotripyrazolo[3,4-f:4′,3′-j:4″,3″-n][1,4]oxazacyclopentadecine;
N²—{(1s,3s)-3-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]cyclobutyl}-N¹,N¹-dimethylethane-1,2-diamine;
(10R,15E)-3,12,14-trimethyl-5,6,9,10,12,18-hexahydro-3H,8H-19,21-etheno-7,10-methanotripyrazolo[3,4-i:3′,4′-m:4″,3″-q][1,8,4]dioxazacyclooctadecin-24-one;
(17E)-16-ethyl-8,10,14-trimethyl-2,9,10,11,12,14-hexahydro-8H-3,5-ethenotripyrazolo[3,4-f:4′,3′-j:4″,3″-n][1,4]oxazacyclopentadecine;
(17E)-10-ethyl-8,14,16-trimethyl-2,9,10,11,12,14-hexahydro-8H-3,5-ethenotripyrazolo[3,4-f:4′,3′-j:4″,3″-n][1,4]oxazacyclopentadecine;
2-[(10S,17E)-8,10,16-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-19-fluoro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(17E)-8,10,16-trimethyl-14-(1-methylpyrrolidin-3-yl)-2,9,10,11,12,14-hexahydro-8H-3,5-ethenotripyrazolo[3,4-f:4′,3′-j:4″,3″-n][1,4]oxazacyclopentadecine;
2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(10R,19E)-8,16,18-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(2S)-1-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]propan-2-ol;
2-[(10S,17E)-19-chloro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2R)-1-[(1S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]propan-2-ol;
2-[(1S,17E)-16-ethyl-8,10,12-trimethyl-2,8,10,11,12,13-hexahydro-14H-3,5-ethenotripyrazolo][3,4-f:3′,4′-j:4″,3″-n][1,4]oxazacyclopentadecin-14-yl|ethan-1-ol;
2-[(10S,17E)-12-ethyl-8,10,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14-tetramethyl-16-[(piperidin-4-yl)oxy]-2,10,11,12,13,14-hexahydro-8H-3,5-ethenotripyrazolo[3,4-f:3′,4′-j:4″,3″-n][1,4]oxazacyclopentadecine;
2-[(10R,19E)-18-ethoxy-8-methyl-2,10,11,12,13,15-hexahydro-3,5-etheno-10,14-methanotripyrazolo[3,4-h:3′,4′-l:4″,3″-p][11,6]oxazacycloheptadecin-16(8H)-yl]ethan-1-ol;
2-[(10S,17E)-6,8,10,12,16-pentamethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,14,16-tetramethyl-12-(oxetan-3-yl)-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-{[1-(methanesulfonyl)piperidin-4-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,13R,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
(10S,13S,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
2-{(10S,17E)-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-[(azetidin-3-yl)oxy]-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-16-{[1-(methanesulfonyl)azetidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(17E)-10-cyclobutyl-8,12,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14-tetramethyl-16-{[(3S)-pyrrolidin-3-yl]oxy}-2,10,11,12,13,14-hexahydro-8H-3,5-ethenotripyrazolo[3,4-f:3′,4′-j:4″,3″-n][1,4]oxazacyclopentadecine;
(10S,17E)-16-{[(3S)-1-(methanesulfonyl)pyrrolidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(2S)-2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(10S,17E)-16-ethoxy-8,10,14-trimethyl-12-(propan-2-yl)-2,10,11,12,13,14-hexahydro-8H-3,5-ethenotripyrazolo[3,4-f:3′,4′-j:4″,3″-n][1,4]oxazacyclopentadecine;
(10S,17E)-8,10,14-trimethyl-2,11,12,14-tetrahydro-8H,10H-3,5-etheno[1,5]dioxacyclopentadecino[10,11-c:15,14-c′:6,7-c″ ]tripyrazole-16-carbonitrile;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10R,19E)-18-ethoxy-8,16-dimethyl-2,8,10,11,12,13,15,16-octahydro-3,5-etheno-10,14-methanotripyrazolo[3,4-h:3′,4′-l:4″,3″-p][1,6]oxazacycloheptadecine;
(10S,17E)-12-cyclopropyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-8,10,12,14-tetramethyl-16-(2,2,2-trifluoroethoxy)-2,10,11,12,13,14-hexahydro-8H-3,5-ethenotripyrazolo[3,4-f:3′,4′-j:4″,3″-n][1,4]oxazacyclopentadecine;
{[(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
(10S,17E)-10,14,16-trimethyl-8-[(methylsulfanyl)methyl]-12-(propan-2-yl)-2,10,11,12,13,14-hexahydro-8H-3,5-ethenotripyrazolo[3,4-f:3′,4′-j:4″,3″-n][1,4]oxazacyclopentadecine;
(10S,17E)-12-ethyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-8-ethyl-10,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-8-ethyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2R)-2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-19-fluoro-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-{[1-(methanesulfonyl)piperidin-4-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,19E)-8,16,18-trimethyl-2,8,10,11,12,13,15,16-octahydro-3,5-etheno-10,14-methanotripyrazolo[3,4-h:3′,4′-l:4″,3″-p][1,6]oxazacycloheptadecine;
2-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10S,17E)-16-{[(3S)-1-(methanesulfonyl)pyrrolidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-bromo-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-16-[(azetidin-3-yl)oxy]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-{[3-(methanesulfonyl)cyclobutyl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
(10R,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
2-[(10R,19E)-18-ethoxy-22-fluoro-8-methyl-2,10,11,12,13,15-hexahydro-3,5-etheno-10,14-methanotripyrazolo[3,4-h:3′,4′-1:4″,3″-p][1,6]oxazacycloheptadecin-16(8H)-yl]ethan-1-ol;
2-{(11S,17E)-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-12-cyclopropyl-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10R,17E)-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(11S,17E)-12-cyclopropyl-16-ethoxy-8,11-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10R,19E)-22-fluoro-8-methyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
(2S)-2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(11S,17E)-6,8,11-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-6,8,10-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
{[(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-cyclopropyl-16-ethoxy-6,8,10-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(11S,17E)-12-cyclopropyl-16-ethoxy-6,8,11-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-ethoxy-12-ethyl-19-fluoro-8,10,14-trimethyl-2,10,11,12,13,14-hexahydro-8H-3,5-ethenotripyrazolo[3,4-f:3′,4′-j:4″,3″-n][1,4]oxazacyclopentadecine;
(2S)-2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10S,17E)-12-ethyl-8,10,14-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
2-[(10S,17E)-19-fluoro-8,10,12-trimethyl-16-(2,2,2-trifluoroethoxy)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10R,17E)-12-cyclopropyl-8,10-dimethyl-6-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-12-cyclopropyl-8,11-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2S)-2-[(10S,17E)-16-ethoxy-19-fluoro-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
1-[(10R,17E)-16-ethoxy-14-(2-hydroxyethyl)-8,10-dimethyl-2,8,10,11,13,14-hexahydro-12H-3,5-ethenotripyrazolo[3,4-f]3′,4′-j:4″,3″-n][1,4]oxazacyclopentadecin-12-yl]ethan-1-one;
ethyl [(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]acetate;
(2S)-2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(10S,17E)-14-(2-hydroxyethyl)-8,10,12-trimethyl-16-[(propan-2-yl)oxy]-2,12,13,14-tetrahydro-8H-3,5-ethenotripyrazolo|3,4-f:3′,4′-j:4″,3″-n∥1,4|oxazacyclopentadecin-11(10H)-one;
2-[(10S,17E)-16-{[1-(methanesulfonyl)azetidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
1-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]-2-methylpropan-2-ol;
2-{(10S,13R,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,13S,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-10-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-11-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2S)-1-{(10R,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
(2S)-2-[(11S,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-[(10R,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(2S)-2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(10S,17E)-12-ethyl-8,10-dimethyl-16-|(propan-2-yl)oxyl-2,8,10,11,12,13-hexahydro-14H-3,5-ethenotripyrazolo[3,4-f:3′,4′-j:4″,3″-n][1,4]oxazacyclopentadecin-14-yl}acetamide;
(10S,17E)-19-fluoro-14-(2-hydroxyethyl)-8,10,12-trimethyl-2-(propan-2-yl)-2,10,11,12,13,14-hexahydro-8H-3,5-ethenotripyrazolo[3,4-f:3′,4′-j:4″,3″-n][1,4]oxazacyclopentadecin-16-ol;
2-[(8aR,19E)-1-(cyclopropylmethoxy)-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
(10R,18E)-17-ethoxy-8,15-dimethyl-2,8,11,12,14,15-hexahydro-10H-3,5-etheno-10,13-methanotripyrazolo[3,4-g:3′,4′-k:4″,3″-o][1,5]oxazacyclohexadecine;
{(17E)-14-(2-hydroxyethyl)-12-(propan-2-yl)-16-[(propan-2-yl)oxy]-2,10,11,12,13,14-hexahydro-7H-3,5-ethenotripyrazolo[3,4-f:3′,4′-j:4″,3″-n][1,4]oxazacyclopentadecin-7-yl}acetonitrile;
(17E)-16-ethoxy-8,14-dimethyl-2,8,10,11,13,14-hexahydro-3,5-etheno-10,12-methanotripyrazolo[3,4-f:3′,4′-j:4″,3″-n][1,4]oxazacyclopentadecine;
2-[(10S,17E)-16-ethoxy-19-fluoro-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(8aR,9R,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-22-fluoro-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(10S,17E)-8-cyclopropyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10S,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
2-{(10R,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(8aR,9S,19E)-9,11-dimethyl-t-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9R,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-22-fluoro-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(10R,17E)-10-(hydroxymethyl)-8,12-dimethyl-16-|(propan-2-yl)oxyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11S,17E)-16-ethoxy-11-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-10-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-(methoxymethyl)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
1-{(10S,17E)-12-ethyl-8,10-dimethyl-16-└(propan-2-yl)oxy┘-2,8,10,11,12,13-hexahydro-14H-3,5-ethenotripyrazolo[3,4-f:3′,4′-j:4″,3″-n][1,4]oxazacyclopentadecin-14-yl}-2-methylpropan-2-ol;
1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-one;
(10R,17E)-14-(2-hydroxyethyl)-8,12-dimethyl-16-[(propan-2-yl)oxy]-2,10,11,12,13,14-hexahydro-8H-3,5-ethenotripyrazolo[3,4-f:3′,4′-j:4″,3″-n][1,4]oxazacyclopentadecine-10-carbonitrile;
2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-16-[(dimethylamino)methyl]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol:
(2R)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
(2S)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
2-[(4aS,7aS,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
2-|(4aR,7aR,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
{[(8aR,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
{[(8aR,9R,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
2-{(10S,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(4aS,7aS,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(4aR,7aR,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(2S)-1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-{(11R,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11R,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(10S,17E)-16-ethoxy-12-ethyl-14-(2-hydroxyethyl)-8,10-dimethyl-2,10,11,12,13,14-hexahydro-8H-3,5-ethenotripyrazolo[3,4-f:3′,4′-j:4″,3″-n][1,4]oxazacyclopentadecine-6-carbonitrile;
2-[(10S,17E)-16-ethoxy-6-ethynyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-(ethylamino)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-1-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-[(10S,17E)-6-amino-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2,2′-[(10S,17E)-10,12-dimethyl-16-[(propan-2-yl)oxy]-10,11,12,13-tetrahydro-8H-3,5-ethenotripyrazolo[3,4-f:3′,4′-j:4″,3″-n][1,4]oxazacyclopentadecine-8,14(2H)-diyl]di(ethan-1-ol);
(17E)-16-ethyl-8,14-dimethyl-2,8,10,11,13,14-hexahydro-3,5-etheno[1,4]dioxacyclopentadecino[10,11-c:15,14-c′:6,7-c″ ]tripyrazole;
2-[(17E)-8,16-dimethyl-2,10,11,13-tetrahydro-3,5-etheno[1,4]dioxacyclopentadecino[10,11-c:15,14-c′:6,7-c″ ]tripyrazol-14(8H)-yl]-N-ethylacetamide;
(17E)-8,14-dimethyl-2,8,10,11,13,14-hexahydro-3,5-etheno[1,4]dioxacyclopentadecino[10,11-c:15,14-c′:6,7-c″ ]tripyrazole-16-carbonitrile;
(17E)-8,14,16-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]thiazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]thiazole-16-carbonitrile; and
(10S,17E)-16-ethyl-8,10,14-trimethyl-2,8,10,11,13,14-hexahydro-3,5-etheno|1,4|dioxacyclopentadecinol10,11-c:15,14-c′:6,7-c″|tripyrazole;
or a pharmaceutically acceptable salt thereof.

34. The compound of clause 1 or 2, selected from the group consisting of (17E)-8,15,16-trimethyl-2,8,9,11,12,15-hexahydro-3,5-etheno[1,4]dioxacyclopentadecino[l11,10-c:15,14-c′:6,7-c″ ]tripyrazole;
(17E)-8,15,16-trimethyl-2,11,12,15-tetrahydro-8H,10H-3,5-etheno[1,5]dioxacyclopentadecino[10,11-c:15,14-c′:6,7-c″ ]tripyrazole;
1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]propan-2-one;
1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-2-methylpropan-2-ol;
2-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]ethan-1-ol;
(17E)-8,16-dimethyl-15-[2-(pyrrolidin-1-yl)ethyl]-2,11,12,15-tetrahydro-8H,10H-3,5-etheno[1,5]dioxacyclopentadecino[10,11-c:15,14-c′:6,7-c″ ]tripyrazole;
(10S,17E)-8,10,15,16-tetramethyl-2,10,11,12,13,15-hexahydro-8H-3,5-ethenotripyrazolo[3,4-f:3′,4′-j:4″,3″-n][1,4]oxazacyclopentadecine;
(17E)-8,15,16-trimethyl-2,10,11,12,13,15-hexahydro-8H-3,5-ethenotripyrazolo[3,4-f:3′,4′-j:4″,3″-n][1,4]oxazacyclopentadecine;
1-[(17E)-8,15,16-trimethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(17E)-8,12,15,16-tetramethyl-2,10,11,12,13,15-hexahydro-8H-3,5-ethenotripyrazolo[3,4-f:3′,4′-j:4″,3″-n][1,4]oxazacyclopentadecine;
1-[(10S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(10S,17E)-8,10,12,15,16-pentamethyl-2,10,11,12,13,15-hexahydro-8H-3,5-ethenotripyrazolo[3,4-f:3′,4′-j:4″,3″-n][1,4]oxazacyclopentadecine;
2-[(10S,17E)-8,10,16-trimethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
2-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
(10S,17E)-12-ethyl-8,10,15,16-tetramethyl-2,10,11,12,13,15-hexahydro-8H-3,5-ethenotripyrazolo|3,4-f:3′,4′-j:4″,3″-n∥1,4|oxazacyclopentadecine;
1-[(10S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]propan-1-one;
2-[(19E)-8,13,18-trimethyl-2,8,11,12,13,14-hexahydro-10H,17H-3,5-etheno[1,7]dioxacycloheptadecino[12,13-c:17,16-c′:8,9-c″ ]tripyrazol-17-yl]ethan-1-ol;
(13E)-3-methyl-3,5,6,7,8,16-hexahydro-9,12-(azeno)-17,19-ethenodipyrazolo[3,4-l:4′,3′-p][1,6]oxazacycloheptadecine;
(18E)-8-methyl-2,8,10,11,12,13-hexahydro-3,5-ethenotripyrazolo[1,5-f:3′,4′-j:4″,3″-n][1,6]oxazacyclopentadecine;
(17E)-15-(2-methoxyethyl)-8,16-dimethyl-2,11,12,15-tetrahydro-8H,10H-3,5-etheno[1,5]dioxacyclopentadecino[10,11-c:15,14-c′:6,7-c″ ]tripyrazole;
2-[(17E)-8,9,16-trimethyl-8,9,11,12-tetrahydro-3,5-etheno[1,4]dioxacyclopentadecino[11,10-c:15,14-c′:6,7-c″ ]tripyrazol-15(2H)-yl]ethan-1-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]piperidin-3-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-1-methylpiperidin-3-ol;
(17E)-8,9,16-trimethyl-15-[2-(pyrrolidin-1-yl)ethyl]-2,8,9,11,12,15-hexahydro-3,5-etheno[1,4]dioxacyclopentadecino[11,10-c:15,14-c′:6,7-c″ ]tripyrazole;
(rac)-1,5-anhydro-2,3-dideoxy-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-L-threo-pentitol;
1,5-anhydro-2,4-dideoxy-2-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-L-threo-pentitol;
(10S,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(10S,17E)-8,10,12,16-tetramethyl-15-[2-(4-methylpiperazin-1-yl)ethyl]-2,10,11,12,13,15-hexahydro-8H-3,5-ethenotripyrazolo[3,4-f:3′,4′-j:4″,3″-n][1,4]oxazacyclopentadecine;
(20E)-8,18-dimethyl-2,8,11,12-tetrahydro-10H-3,5-ethenodipyrazolo[3″,4″:10′,11′;4′″,3′″:14′,15′][1,5]dioxacyclopentadecino[6′,7′:3,4]pyrazolo[1, 5-a]pyrazin-19(18H)-one;
(10S,17E)-8,10,12,16-tetramethyl-15-[2-(morpholin-4-yl)ethyl]-2,10,11,12,13,15-hexahydro-8H-3,5-ethenotripyrazolo[3,4-f:3′,4′-j:4″,3″-n][1,4]oxazacyclopentadecine;
4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
N¹—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]cyclobutyl}-N²,N²,N²-trimethylethane-1,2-diamine;
2-methyl-4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
2-methyl-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
N²—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]cyclobutyl}-N¹,N¹-dimethylethane-1,2-diamine;
(17E)-8,10,16-trimethyl-15-(1-methylpyrrolidin-3-yl)-2,9,10,11,12,15-hexahydro-8H-3,5-ethenotripyrazolo[3,4-f:4′,3′-j:4″,3″-n][1,4]oxazacyclopentadecine;
(10R,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(2S)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,15-pentamethyl-2,10,11,12,13,15-hexahydro-8H-3,5-ethenotripyrazolo[4,3-f:3′,4′-j:4″,3″-n][1,4]oxazacyclopentadecine;
(2R)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,16-pentamethyl-2,10,11,12,13,16-hexahydro-8H-3,5-ethenotripyrazolo[4,3-f:3′,4′-j:4″,3″-n][1,4]oxazacyclopentadecine;
(10S,17E)-8,10,12,16-tetramethyl-2,8,10,11,12,13-hexahydro-3,5-etheno[1,2]oxazolo[3,4-f]dipyrazolo[3,4-j:4′,3′-n][1,4]oxazacyclopentadecine;
(10S,17E)-8,10,12,16-tetramethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12,16-tetramethyl-2,8,10,11,12,13-hexahydro-3,5-ethenodipyrazolo[3,4-j:4′,3′-n][1,2]thiazolo[3,4-f][1,4]oxazacyclopentadecine;
(10S,20E)-18-[2-(methanesulfonyl)ethyl]-8,10,12-trimethyl-2,8,10,11,12,13,16,17,18,19-decahydro-3,5-ethenopyrazino[1′,2′:1,5]pyrazolo [3,4-f]dipyrazolo[3,4-j:4′,3′-n][1,4]oxazacyclopentadecine;
(10S,17E)-16-cyclopropyl-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethyl-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethoxy-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10R,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]oxazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole-16-carbonitrile;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]thiazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]thiazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]oxazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]thiazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10-dimethyl-2,8,10,11-tetrahydro-13H-3,5-ethenodipyrazolo|3′,4′:10,11;4″,3″:14,15∥1,4|dioxacyclopentadecino|6,7-c∥1,2|thiazole-16-carbonitrile;
(17E)-8,16-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole;
(17E)-8,16-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole; and
(17E)-8,16-dimethyl-2,8,10,11-tetrahydro-13H-3,5-ethenodipyrazolo[3′,4′:10,11;4″,3″:14,15][1,4]dioxacyclopentadecino[6,7-c][1,2]thiazole;
or a pharmaceutically acceptable salt thereof.

The following represent illustrative embodiments of compounds of Formula (1)-(X11):

| Ex # | Structure | Name |
|---|---|---|
| 1 | 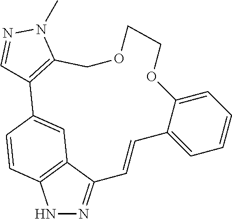 [SMILES: Cn1ncc2c1COCCOc1ccccc1/C=C/c1n[nH]c3ccc-2cc13] | (11E)-1-methyl-1,18,19,21-tetrahydro- 8H-10,7,4- (ethan[1]yl[1,2]diylidene)pyrazolo[3,4- f][1,4,12,13]benzodioxadiazacyclooctadecine |
| 2 | 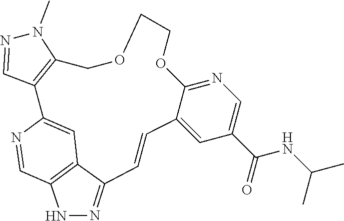 [SMILES: CC(C)NC(=O)c1cnc2c(c1)/C=C/c1n[nH]c3cnc(cc13)-c1cnn(C)c1COCCO2] | (18E)-8-methyl-N-(propan-2-yl)- 8,9,11,12-tetrahydro-2H-3,5- ethenodipyrazolo[3′,4′:9,10;4″,3″:13,14] [1,4]dioxacyclopentadecino[5,6- b]pyridine-16-carboxamide |
| 3 | 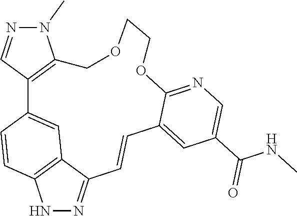 [SMILES: CNC(=O)c1cnc2c(c1)/C=C/c1n[nH]c3ccc(cc13)-c1cnn(C)c1COCCO2] | (18E)-N,8-dimethyl-8,9,11,12-tetrahydro- 2H-3,5- ethenodipyrazolo[3′,4′:9,10;4″,3″:13,14] [1,4]dioxacyclopentadecino[5,6- b]pyridine-16-carboxamide |
| 4 | 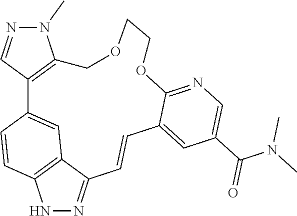 [SMILES: CN(C)C(=O)c1cnc2c(c1)/C=C/c1n[nH]c3ccc(cc13)-c1cnn(C)c1COCCO2] | (18E)-N,N,8-trimethyl-8,9,11,12- tetrahydro-2H-3,5- ethenodipyrazolo[3′,4′:9,10;4″,3″:13,14] [1,4]dioxacyclopentadecino[5,6- b]pyridine-16-carboxamide |
| 5 | 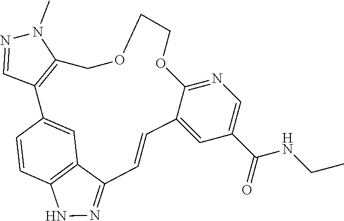 [SMILES: CCNC(=O)c1cnc2c(c1)/C=C/c1n[nH]c3ccc(cc13)-c1cnn(C)c1COCCO2] | (18E)-N-ethyl-8-methyl-8,9,11,12- tetrahydro-2H-3,5- ethenodipyrazolo[3′,4′:9,10;4″,3″:13,14] [1,4]dioxacyclopentadecino[5,6- b]pyridine-16-carboxamide |
| 6 | 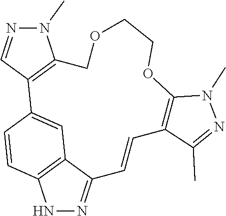 [SMILES: Cc1nn(C)c2c1/C=C/c1n[nH]c3ccc(cc13)-c1cnn(C)c1COCCO2] | (17E)-8,14,16-trimethyl-2,8,9,11,12,14- hexahydro-3,5- etheno[1,4]dioxacyclopentadecino[11,10- c:15,14-c′:6,7-c″]tripyrazole |
| 7 | 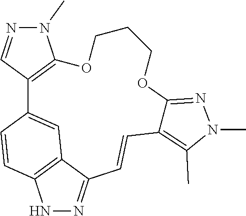 [SMILES: Cc1c2c(nn1C)OCCCOc1c(cnn1C)-c1ccc3[nH]nc(c3c1)/C=C/2] | (17E)-8,15,16-trimethyl-2,8,9,11,12,15- hexahydro-3,5- etheno[1,4]dioxacyclopentadecino[11,10- c:15,14-c′:6,7-c″]tripyrazole |
| 8 | 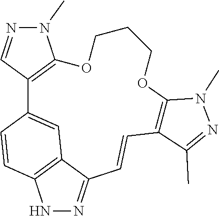 [SMILES: Cc1nn(C)c2c1/C=C/c1n[nH]c3ccc(cc13)-c1cnn(C)c1OCCCO2] | (17E)-8,14,16-trimethyl-2,11,12,14- tetrahydro-8H,10H-3,5- etheno[1,5]dioxacyclopentadecino[10,11- c:15,14-c′:6,7-c″]tripyrazole |
| 9 | 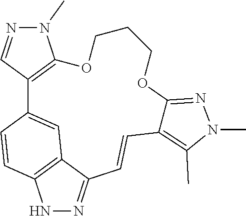 [SMILES: Cc1c2c(nn1C)OCCCOc1c(cnn1C)-c1ccc3[nH]nc(c3c1)/C=C/2] | (17E)-8,15,16-trimethyl-2,11,12,15- tetrahydro-8H,10H-3,5- etheno[1,5]dioxacyclopentadecino[10,11- c:15,14-c′:6,7-c″]tripyrazole |
| 10 | 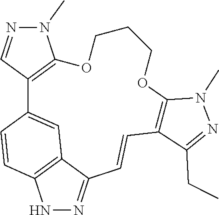 [SMILES: CCc1nn(C)c2c1/C=C/c1n[nH]c3ccc(cc13)-c1cnn(C)c1OCCCO2] | (17E)-16-ethyl-8,14-dimethyl- 2,11,12,14-tetrahydro-8H,10H-3,5- etheno[1,5]dioxacyclopentadecino[10,11- c:15,14-c′:6,7-c″]tripyrazole |
| 11 | 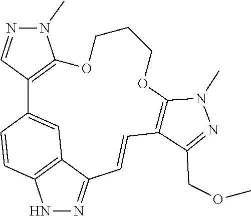 [SMILES: COCc1nn(C)c2c1/C=C/c1n[nH]c3ccc(cc13)-c1cnn(C)c1OCCCO2] | (17E)-16-(methoxymethyl)-8,14- dimethyl-2,11,12,14-tetrahydro-8H,10H- 3,5- etheno[1,5]dioxacyclopentadecino[10,11- c:15,14-c′:6,7-c″]tripyrazole |
| 12 | 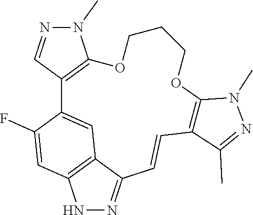 [SMILES: Cc1nn(C)c2c1/C=C/c1n[nH]c3cc(F)c(cc13)-c1cnn(C)c1OCCCO2] | (17E)-19-fluoro-8,14,16-trimethyl- 2,11,12,14-tetrahydro-8H,10H-3,5- etheno[1,5]dioxacyclopentadecino[10,11- c:15,14-c′:6,7-c″]tripyrazole |
| 13 | 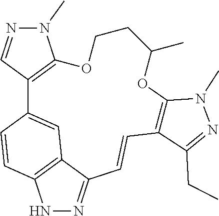 [SMILES: CCc1nn(C)c2c1/C=C/c1n[nH]c3ccc(cc13)-c1cnn(C)c1OCCC(C)O2] | (17E)-16-ethyl-8,12,14-trimethyl- 2,11,12,14-tetrahydro-8H,10H-3,5- etheno[1,5]dioxacyclopentadecino[10,11- c:15,14-c′:6,7-c″]tripyrazole |
| 14 | 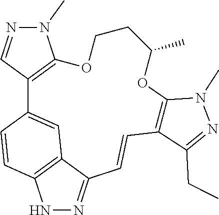 [SMILES: CCc1nn(C)c2c1/C=C/c1n[nH]c3ccc(cc13)-c1cnn(C)c1OCC[C@H](C)O2] | (12S,17E)-16-ethyl-8,12,14-trimethyl- 2,11,12,14-tetrahydro-8H,10H-3,5- etheno[1,5]dioxacyclopentadecino[10,11- c:15,14-c′:6,7-c″]tripyrazole |
| 15 | 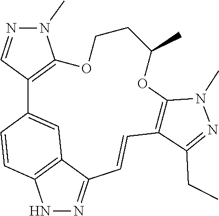 [SMILES: CCc1nn(C)c2c1/C=C/c1n[nH]c3ccc(cc13)-c1cnn(C)c1OCC[C@@H](C)O2] | (12R,17E)-16-ethyl-8,12,14-trimethyl- 2,11,12,14-tetrahydro-8H,10H-3,5- etheno[1,5]dioxacyclopentadecino[10,11- c:15,14-c′:6,7-c″]tripyrazole |
| 16 | 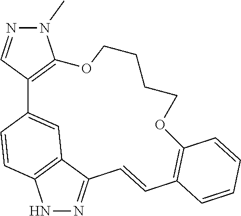 [SMILES: Cn1ncc2c1OCCCCOc1ccccc1/C=C/c1n[nH]c3ccc-2cc13] | (11E)-1-methyl-18,19,20,21-tetrahydro- 1H,8H-10,7,4- (ethan[1]yl[1,2]diylidene)pyrazolo[3,4- g][1,6,13,14]benzodioxadiazacyclononad ecine |
| 17 | 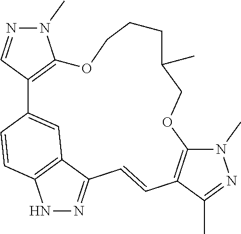 [SMILES: Cc1nn(C)c2c1/C=C/c1n[nH]c3ccc(cc13)-c1cnn(C)c1OCCCC(C)CO2] | (19E)-8,13,16,18-tetramethyl- 2,11,12,13,14,16-hexahydro-8H,10H-3,5- etheno[1,7]dioxacycloheptadecino[12,13- c:17,16-c′:8,9-c″]tripyrazole |
| 18 | 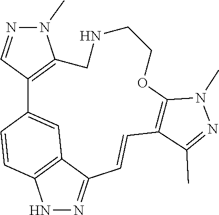 [SMILES: Cc1nn(C)c2c1/C=C/c1n[nH]c3ccc(cc13)-c1cnn(C)c1CNCCO2] | (17E)-8,14,16-trimethyl-2,9,10,11,12,14- hexahydro-8H-3,5-ethenotripyrazolo[3,4- f:4′,3′-j:4″,3″- n][1,4]oxazacyclopentadecine |
| 19 | 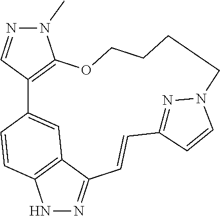 [SMILES: Cn1ncc2c1OCCCCn1ccc(n1)/C=C/c1n[nH]c3ccc-2cc13] | (13E)-3-methyl-3,5,6,7,8,16-hexahydro- 9,12-(azeno)-17,19- ethenodipyrazolo[3,4-l:4′,3′- p][1,6]oxazacycloheptadecine |
| 20 | 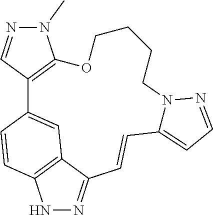 [SMILES: Cn1ncc2c1OCCCCn1nccc1/C=C/c1n[nH]c3ccc-2cc13] | (18E)-8-methyl-2,8,10,11,12,13- hexahydro-3,5-ethenotripyrazolo[1,5- f:3′,4′-j:4″,3″- n][1,6]oxazacyclopentadecine |
| 21 | 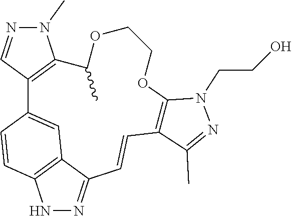 [SMILES: Cc1nn(CCO)c2c1/C=C/c1n[nH]c3ccc(cc13)-c1cnn(C)c1C(C)OCCO2] | 2-[(17E)-8,9,16-trimethyl-8,9,11,12- tetrahydro-3,5- etheno[1,4]dioxacyclopentadecino[11,10- c:15,14-c′:6,7-c″]tripyrazol-14(2H)- yl]ethan-1-ol |
| 22 | 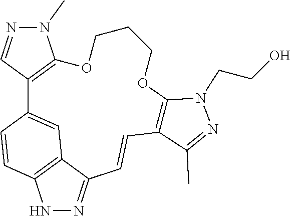 [SMILES: Cc1nn(CCO)c2c1/C=C/c1n[nH]c3ccc(cc13)-c1cnn(C)c1OCCCO2] | 2-[(17E)-8,16-dimethyl-2,8,11,12- tetrahydro-10H,14H-3,5-etheno[1,5]dioxacyclopentadecino[10,11- c:15,14-c′:6,7-c″]tripyrazol-14-yl]ethan- 1-ol |
| 23 | 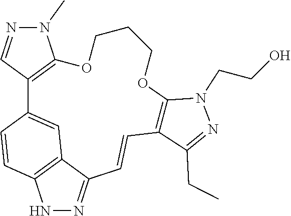 [SMILES: CCc1nn(CCO)c2c1/C=C/c1n[nH]c3ccc(cc13)-c1cnn(C)c1OCCCO2] | 2-[(17E)-16-ethyl-8-methyl-2,8,11,12- tetrahydro-10H,14H-3,5- etheno[1,5]dioxacyclopentadecino[10,11- c:15,14-c′:6,7-c″]tripyrazol-14-yl]ethan- 1-ol |
| 24 | 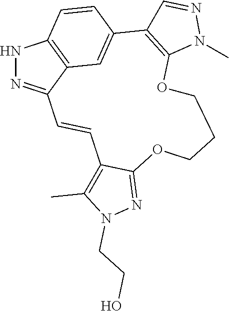 [SMILES: Cc1c2c(nn1CCO)OCCCOc1c(cnn1C)-c1ccc3[nH]nc(c3c1)/C=C/2] | 2-[(17E)-8,16-dimethyl-2,8,11,12- tetrahydro-10H,15H-3,5- etheno[1,5]dioxacyclopentadecino[10,11- c:15,14-c′:6,7-c″]tripyrazol-15-yl]ethan- 1-ol |
| 25 | 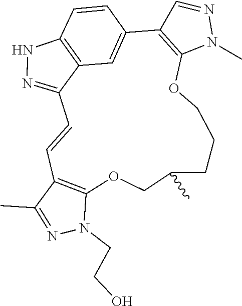 | 2-[(19E)-8,13,18-trimethyl- 2,8,11,12,13,14-hexahydro-10H,16H-3,5- etheno[1,7]dioxacycloheptadecino[12,13- c:17,16-c′:8,9-c″]tripyrazol-16-yl]ethan- 1-ol |
| 26 | 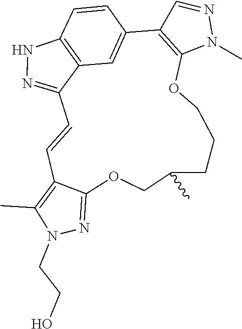 | 2-[(19E)-8,13,18-trimethyl- 2,8,11,12,13,14-hexahydro-10H,17H-3,5- etheno[1,7]dioxacycloheptadecino[12,13- c:17,16-c′:8,9-c″]tripyrazol-17-yl]ethan- 1-ol |
| 27 | 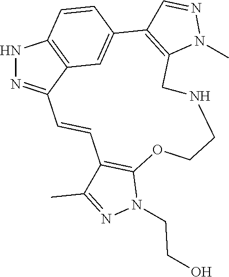 [SMILES: Cc1nn(CCO)c2c1/C=C/c1n[nH]c3ccc(cc13)-c1cnn(C)c1CNCCO2] | 2-[(17E)-8,16-dimethyl-2,8,9,10,11,12- hexahydro-14H-3,5- ethenotripyrazolo[3,4-f:4′,3′-j:4″,3″- n][1,4]oxazacyclopentadecin-14- yl]ethan-1-ol |
| 28 | 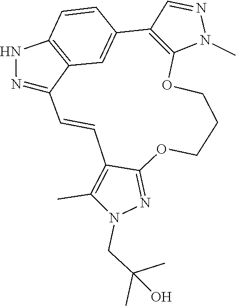 [SMILES: Cc1c2c(nn1CC(C)(C)O)OCCCOc1c(cnn1C)-c1ccc3[nH]nc(c3c1)/C=C/2] | 1-[(17E)-8,16-dimethyl-2,8,11,12- tetrahydro-10H,15H-3,5- etheno[1,5]dioxacyclopentadecino[10,11- c:15,14-c′:6,7-c″]tripyrazol-15-yl]-2- methylpropan-2-ol |
| 29 | 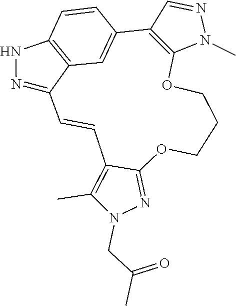 [SMILES: CC(=O)Cn1nc2c(c1C)/C=C/c1n[nH]c3ccc(cc13)-c1cnn(C)c1OCCCO2] | 1-[(17E)-8,16-dimethyl-2,8,11,12- tetrahydro-10H,15H-3,5- etheno[1,5]dioxacyclopentadecino[10,11- c:15,14-c′:6,7-c″]tripyrazol-15-yl]propan- 2-one |
| 30 | 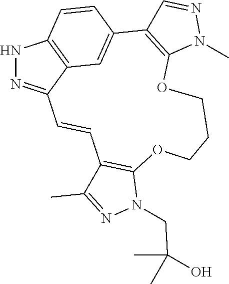 [SMILES: Cc1nn(CC(C)(C)O)c2c1/C=C/c1n[nH]c3ccc(cc13)-c1cnn(C)c1OCCCO2] | 1-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14-yl]-2- methylpropan-2-ol |
| 31 | 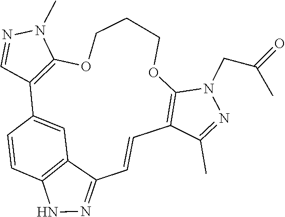 | 1-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14-yl]propan- 2-one |
| 32 | 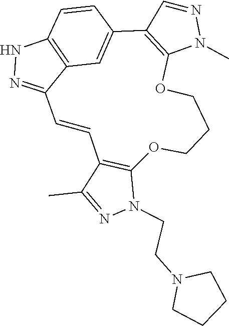 [SMILES: Cc1nn(CCN2CCCC2)c2c1/C=C/c1n[nH]c3ccc(cc13)-c1cnn(C)c1OCCCO2] | (17E)-8,16-dimethyl-14-[2-(pyrrolidin-1- yl)ethyl]-2,11,12,14-tetrahydro-8H,10H- 3,5- etheno[1,5]dioxacyclopentadecino[10,11- c:15,14-c′:6,7-c″]tripyrazole |
| 33 | 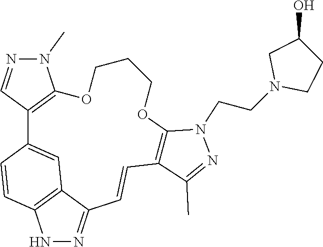 [SMILES: Cc1nn(CCN2CC[C@H](O)C2)c2c1/C=C/c1n[nH]c3ccc(cc13)-c1cnn(C)c1OCCCO2] | (3S)-1-{2-[(17E)-8,16-dimethyl- 2,8,11,12-tetrahydro-10H,14H-3,5- etheno[1,5]dioxacyclopentadecino[10,11- c:15,14-c′:6,7-c″]tripyrazol-14- yl]ethyl}pyrrolidin-3-ol |
| 34 | 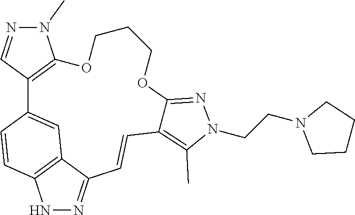 [SMILES: Cc1c2c(nn1CCN1CCCC1)OCCCOc1c(cnn1C)-c1ccc3[nH]nc(c3c1)/C=C/2] | (17E)-8,16-dimethyl-15-[2-(pyrrolidin-1- yl)ethyl]-2,11,12,15-tetrahydro-8H,10H- 3,5- etheno[1,5]dioxacyclopentadecino[10,11- c:15,14-c′:6,7-c″]tripyrazole |
| 35 | 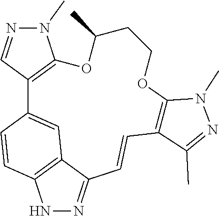 [SMILES: Cc1nn(C)c2c1/C=C/c1n[nH]c3ccc(cc13)-c1cnn(C)c1O[C@@H](C)CCO2] | (10S,17E)-8,10,14,16-tetramethyl- 2,11,12,14-tetrahydro-8H,10H-3,5- etheno[1,5]dioxacyclopentadecino[10,11- c:15,14-c′:6,7-c″]tripyrazole |
| 36 | 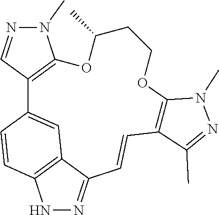 [SMILES: Cc1nn(C)c2c1/C=C/c1n[nH]c3ccc(cc13)-c1cnn(C)c1O[C@H](C)CCO2] | (10R,17E)-8,10,14,16-tetramethyl- 2,11,12,14-tetrahydro-8H,10H-3,5- etheno[1,5]dioxacyclopentadecino[10,11- c:15,14-c′:6,7-c″]tripyrazole |
| 37 | 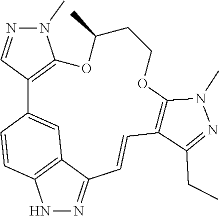 [SMILES: CCc1nn(C)c2c1/C=C/c1n[nH]c3ccc(cc13)-c1cnn(C)c1O[C@@H](C)CCO2] | (10S,17E)-16-ethyl-8,10,14-trimethyl- 2,11,12,14-tetrahydro-8H,10H-3,5- etheno[1,5]dioxacyclopentadecino[10,11- c:15,14-c′:6,7-c″]tripyrazole |
| 38 | 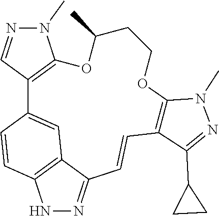 [SMILES: C[C@H]1CCOc2c(c(C3CC3)nn2C)/C=C/c2n[nH]c3ccc(cc23)-c2cnn(C)c2O1] | (10S,17E)-16-cyclopropyl-8,10,14- trimethyl-2,11,12,14-tetrahydro-8H,10H- 3,5- etheno[1,5]dioxacyclopentadecino[10,11- c:15,14-c′:6,7-c″]tripyrazole |
| 39 | 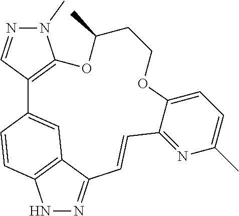 [SMILES: Cc1ccc2c(n1)/C=C/c1n[nH]c3ccc(cc13)-c1cnn(C)c1O[C@@H](C)CCO2] | (10S,18E)-8,10,16-trimethyl-2,8,11,12- tetrahydro-10H-3,5- ethenodipyrazolo[3′,4′:10,11;4″,3″:14,15] [1,5]dioxacyclopentadecino[7,6- b]pyridine |
| 40 | 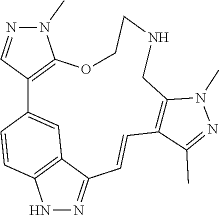 [SMILES: Cc1nn(C)c2c1/C=C/c1n[nH]c3ccc(cc13)-c1cnn(C)c1OCCNC2] | (17E)-8,14,16-trimethyl- 2,10,11,12,13,14-hexahydro-8H-3,5- ethenotripyrazolo[3,4-f:3′,4′-j:4″,3″- n][1,4]oxazacyclopentadecine |
| 41 | 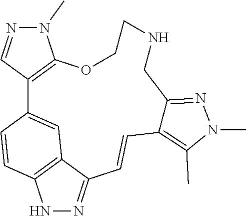 [SMILES: Cc1c2c(nn1C)CNCCOc1c(cnn1C)-c1ccc3[nH]nc(c3c1)/C=C/2] | (17E)-8,15,16-trimethyl- 2,10,11,12,13,15-hexahydro-8H-3,5- ethenotripyrazolo[3,4-f:3′,4′-j:4″,3″′- n][1,4]oxazacyclopentadecine |
| 42 | 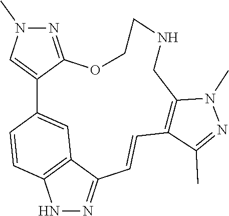 | (17E)-7,14,16-trimethyl- 2,10,11,12,13,14-hexahydro-7H-3,5- ethenotripyrazolo[3,4-f:3′,4′-j:4″,3″- n][1,4]oxazacyclopentadecine |
| 43 | 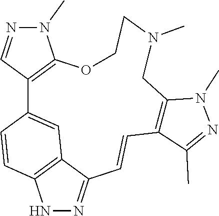 [SMILES: Cc1nn(C)c2c1/C=C/c1n[nH]c3ccc(cc13)-c1cnn(C)c1OCCN(C)C2] | (17E)-8,12,14,16-tetramethyl- 2,10,11,12,13,14-hexahydro-8H-3,5- ethenotripyrazolo[3,4-f:3′,4′-j:4″,3″- n][1,4]oxazacyclopentadecine |
| 44 | 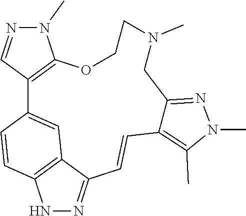 [SMILES: Cc1c2c(nn1C)CN(C)CCOc1c(cnn1C)-c1ccc3[nH]nc(c3c1)/C=C/2] | (17E)-8,12,15,16-tetramethyl- 2,10,11,12,13,15-hexahydro-8H-3,5- ethenotripyrazolo[3,4-f:3′,4′-j:4″,3″- n][1,4]oxazacyclopentadecine |
| 45 | 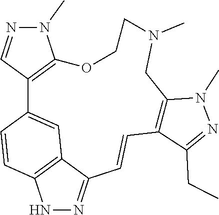 [SMILES: CCc1nn(C)c2c1/C=C/c1n[nH]c3ccc(cc13)-c1cnn(C)c1OCCN(C)C2] | (17E)-16-ethyl-8,12,14-trimethyl- 2,10,11,12,13,14-hexahydro-8H-3,5- ethenotripyrazolo[3,4-f:3′,4′-j:4″,3″- n][1,4]oxazacyclopentadecine |
| 46 | 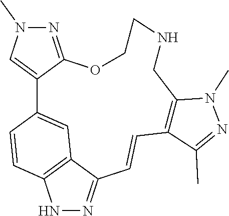 | (17E)-7,12,14,16-tetramethyl- 2,10,11,12,13,14-hexahydro-7H-3,5- ethenotripyrazolo[3,4-f:3′,4′-j:4″,3″- n][1,4]oxazacyclopentadecine |
| 47 | 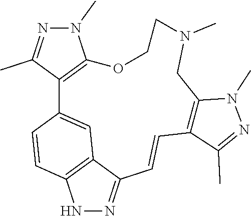 | (17E)-6,8,12,14,16-pentamethyl- 2,10,11,12,13,14-hexahydro-8H-3,5- ethenotripyrazolo[3,4-f:3′,4′-j:4″,3″- n][1,4]oxazacyclopentadecine |
| 48 | 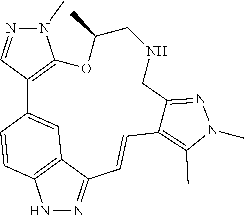 [SMILES: Cc1c2c(nn1C)CNC[C@H](C)Oc1c(cnn1C)-c1ccc3[nH]nc(c3c1)/C=C/2] | (10S,17E)-8,10,15,16-tetramethyl- 2,10,11,12,13,15-hexahydro-8H-3,5- ethenotripyrazolo[3,4-f:3′,4′-j:4″,3″- n][1,4]oxazacyclopentadecine |
| 49 | 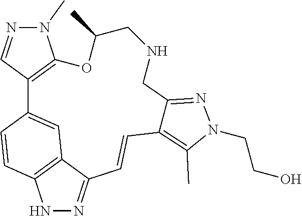 [SMILES: Cc1c2c(nn1CCO)CNC[C@H](C)Oc1c(cnn1C)-c1ccc3[nH]nc(c3c1)/C=C/2] | yl]ethan-1-ol 2-[(10S,17E)-8,10,16-trimethyl-2,8,10,11,12,13-hexahydro-15H-3,5- ethenotripyrazolo[3,4-f:3′,4′-j:4″,3″- n][1,4]oxazacyclopentadecin-15- |
| 50 | 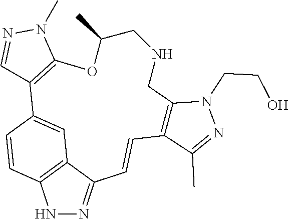 [SMILES: Cc1nn(CCO)c2c1/C=C/c1n[nH]c3ccc(cc13)-c1cnn(C)c1O[C@@H](C)CNC2] | 2-[(10S,17E)-8,10,16-trimethyl- 2,8,10,11,12,13-hexahydro-15H-3,5- ethenotripyrazolo[3,4-f:3′,4′-j:4″,3″-n] [1,4]oxazacyclopentadecin-14- yl]ethan-1-ol |
| 51 | 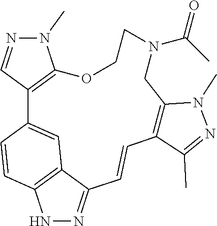 [SMILES: CC(=O)N1CCOc2c(cnn2C)-c2ccc3[nH]nc(c3c2)/C=C/c2c(C)nn(C)c2C1] | 1-[(17E)-8,14,16-trimethyl- 2,8,10,11,13,14-hexahydro-12H-3,5- ethenotripyrazolo[3,4-f:3′,4′-j:4″,3″- n][1,4]oxazacyclopentadecin-12- yl]ethan-1-one |
| 52 | 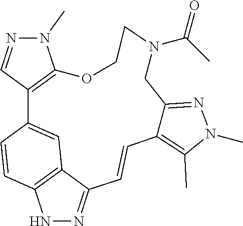 [SMILES: CC(=O)N1CCOc2c(cnn2C)-c2ccc3[nH]nc(c3c2)/C=C/c2c(nn(C)c2C)C1] | 1-[(17E)-8,15,16-trimethyl- 2,8,10,11,13,15-hexahydro-12H-3,5- ethenotripyrazolo[3,4-f:3′,4′-j:4″,3″- n][1,4]oxazacyclopentadecin-12- yl]ethan-1-one |
| 53 | 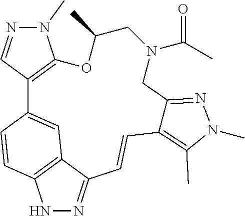 [SMILES: CC(=O)N1Cc2nn(C)c(C)c2/C=C/c2n[nH]c3ccc(cc23)-c2cnn(C)c2O[C@@H](C)C1] | 1-[(10S,17E)-8,10,15,16-tetramethyl- 2,8,10,11,13,15-hexahydro-12H-3,5- ethenotripyrazolo[3,4-f:3′,4′-j:4″,3″′- n][1,4]oxazacyclopentadecin-12- yl]ethan-1-one |
| 54 | 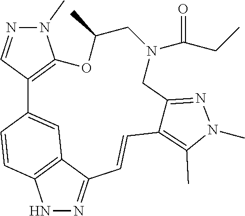 [SMILES: CCC(=O)N1Cc2nn(C)c(C)c2/C=C/c2n[nH]c3ccc(cc23)-c2cnn(C)c2O[C@@H](C)C1] | 1-[(10S,17E)-8,10,15,16-tetramethyl- 2,8,10,11,13,15-hexahydro-12H-3,5- ethenotripyrazolo[3,4-f:3′,4′-j:4″,3″- n][1,4]oxazacyclopentadecin-12- yl]propan-1-one |
| 55 | 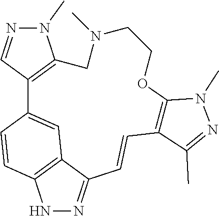 [SMILES: Cc1nn(C)c2c1/C=C/c1n[nH]c3ccc(cc13)-c1cnn(C)c1CN(C)CCO2] | (17E)-8,10,14,16-tetramethyl- 2,9,10,11,12,14-hexahydro-8H-3,5- ethenotripyrazolo[3,4-f:4′,3′-j:4″,3″- n][1,4]oxazacyclopentadecine |
| 56 | 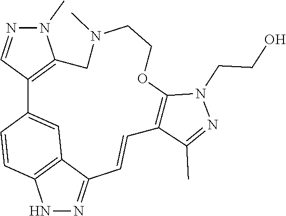 [SMILES: Cc1nn(CCO)c2c1/C=C/c1n[nH]c3ccc(cc13)-c1cnn(C)c1CN(C)CCO2] | 2-[(17E)-8,10,16-trimethyl- 2,8,9,10,11,12-hexahydro-14H-3,5- ethenotripyrazolo[3,4-f:4′,3′-j:4″,3″- n][1,4]oxazacyclopentadecin-14- yl]ethan-1-ol |
| 57 | 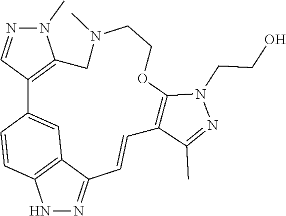 [SMILES: Cc1nn(CCO)c2c1/C=C/c1n[nH]c3ccc(cc13)-c1cnn(C)c1CN(C)CCO2] | 2-[(17E)-10-ethyl-8,16-dimethyl- 2,8,9,10,11,12-hexahydro-14H-3,5- ethenotripyrazolo[3,4-f:4′,3′-j:4″,3″- n][1,4]oxazacyclopentadecin-14- yl]ethan-1-ol |
| 58 | 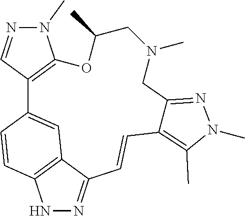 [SMILES: Cc1c2c(nn1C)CN(C)C[C@H](C)Oc1c(cnn1C)-c1ccc3[nH]nc(c3c1)/C=C/2] | (10S,17E)-8,10,12,15,16-pentamethyl- 2,10,11,12,13,15-hexahydro-8H-3,5- ethenotripyrazolo[3,4-f:3′,4′-j:4″,3″- n][1,4]oxazacyclopentadecine |
| 59 | 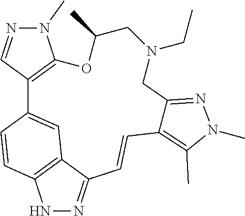 [SMILES: CCN1Cc2nn(C)c(C)c2/C=C/c2n[nH]c3ccc(cc23)-c2cnn(C)c2O[C@@H](C)C1] | (10S,17E)-12-ethyl-8,10,15,16- tetramethyl-2,10,11,12,13,15-hexahydro- 8H-3,5-ethenotripyrazolo[3,4-f:3′,4′- j:4″,3″-n][1,4]oxazacyclopentadecine |
| 60 | 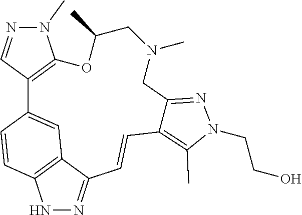 [SMILES: Cc1c2c(nn1CCO)CN(C)C[C@H](C)Oc1c(cnn1C)-c1ccc3[nH]nc(c3c1)/C=C/2] | 2-[(10S,17E)-8,10,12,16-tetramethyl- 2,8,10,11,12,13-hexahydro-15H-3,5- ethenotripyrazolo[3,4-f:3′,4′-j:4″,3″- n][1,4]oxazacyclopentadecin-15- yl]ethan-1-ol |
| 61 | 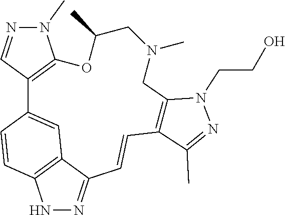 [SMILES: Cc1nn(CCO)c2c1/C=C/c1n[nH]c3ccc(cc13)-c1cnn(C)c1O[C@@H](C)CN(C)C2] | 2-[(10S,17E)-8,10,12,16-tetramethyl- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 62 | 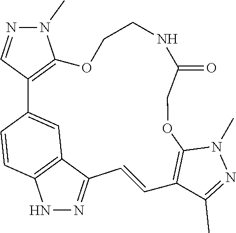 [SMILES: Cc1nn(C)c2c1/C=C/c1n[nH]c3ccc(cc13)-c1cnn(C)c1OCCNC(=O)CO2] | (19E)-8,16,18-trimethyl-2,11,12,16- tetrahydro-8H,10H-3,5- ethenotripyrazolo[3,4-h:3′,4′-l:4″,3″- p][1,7,4]dioxazacycloheptadecin- 13(14H)-one |
| 63 | 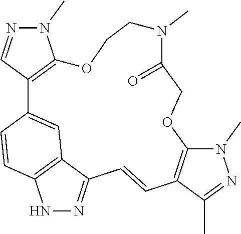 [SMILES: Cc1nn(C)c2c1/C=C/c1n[nH]c3ccc(cc13)-c1cnn(C)c1OCCN(C)C(=O)CO2] | (19E)-8,12,16,18-tetramethyl-2,11,12,16- tetrahydro-8H,10H-3,5- ethenotripyrazolo[3,4-h:3′,4′-l:4″,3″- p][1,7,4]dioxazacycloheptadecin- 13(14H)-one |
| 64 | 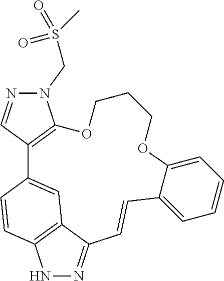 [SMILES: CS(=O)(=O)Cn1ncc2c1OCCCOc1ccccc1/C=C/c1n[nH]c3ccc-2cc13] | (11E)-1-[(methanesulfonyl)methyl]- 19,20-dihydro-1H,8H,18H-10,7,4- (ethan[1]yl[1,2]diylidene)pyrazolo[3,4- f][1,5,12,13]benzodioxadiazacyclooctadecine |
| 65 | 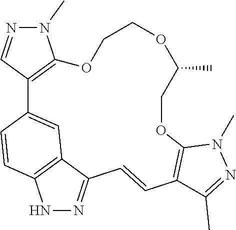 [SMILES: Cc1nn(C)c2c1/C=C/c1n[nH]c3ccc(cc13)-c1cnn(C)c1OCCO[C@H](C)CO2] | (13R,19E)-8,13,16,18-tetramethyl- 2,10,11,13,14,16-hexahydro-8H-3,5- etheno[1,4,7]trioxacycloheptadecino[12,1 3-c:17,16-c′:8,9-c″]tripyrazole |
| 66 | 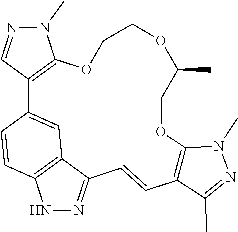 [SMILES: Cc1nn(C)c2c1/C=C/c1n[nH]c3ccc(cc13)-c1cnn(C)c1OCCO[C@@H](C)CO2] | (13S,19E)-8,13,16,18-tetramethyl- 2,10,11,13,14,16-hexahydro-8H-3,5- etheno[1,4,7]trioxacycloheptadecino[12,1 3-c:17,16-c′:8,9-c″]tripyrazole |
| 67 | 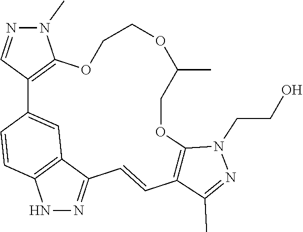 [SMILES: Cc1nn(CCO)c2c1/C=C/c1n[nH]c3ccc(cc13)-c1cnn(C)c1OCCOC(C)CO2] | 2-[(19E)-8,13,18-trimethyl- 2,8,10,11,13,14-hexahydro-16H-3,5- etheno[1,4,7]trioxacycloheptadecino[12,1 3-c:17,16-c′:8,9-c″]tripyrazol-16- yl]ethan-1-ol |
| 68 | 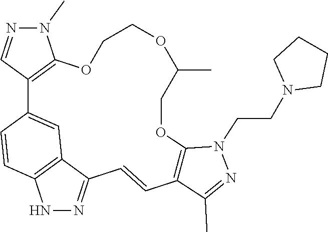 [SMILES: Cc1nn(CCN2CCCC2)c2c1/C=C/c1n[nH]c3ccc(cc13)-c1cnn(C)c1OCCOC(C)CO2] | (19E)-8,13,18-trimethyl-16-[2- (pyrrolidin-1-yl)ethyl]-2,10,11,13,14,16- hexahydro-8H-3,5- etheno[1,4,7]trioxacycloheptadecino[12,1 3-c:17,16-c′:8,9-c″]tripyrazole |
| 69 | 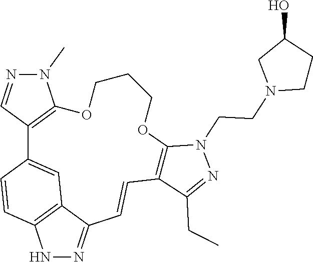 [SMILES: CCc1nn(CCN2CC[C@H](O)C2)c2c1/C=C/c1n[nH]c3ccc(cc13)-c1cnn(C)c1OCCCO2] | (3S)-1-{2-[(17E)-16-ethyl-8-methyl- 2,8,11,12-tetrahydro-10H,14H-3,5- etheno[1,5]dioxacyclopentadecino[10,11- c:15,14-c′:6,7-c″]tripyrazol-14- yl]ethyl}pyrrolidin-3-ol |
| 70 | 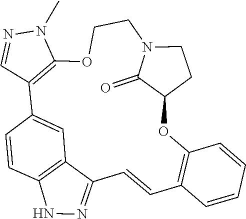 [SMILES: Cn1ncc2c1OCCN1CC[C@@H](Oc3ccccc3/C=C/c3n[nH]c4ccc-2cc34)C1=O] | (10R,16E)-3-methyl-3,5,6,9,10,19- hexahydro-8H-20,22-etheno-7,10- methanodipyrazolo[3,4-i:4′,3′- m][1,8,5]benzodioxazacyclooctadecin- 25-one |
| 71 | 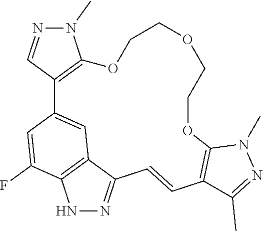 [SMILES: Cc1nn(C)c2c1/C=C/c1n[nH]c3c(F)cc(cc13)-c1cnn(C)c1OCCOCCO2] | (19E)-22-fluoro-8,16,18-trimethyl- 2,10,11,13,14,16-hexahydro-8H-3,5- etheno[1,4,7]trioxacycloheptadecino[12,1 3-c:17,16-c′:8,9-c″]tripyrazole |
| 72 | 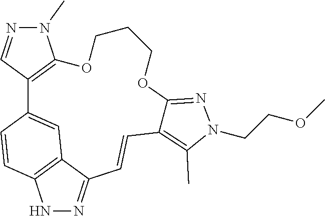 [SMILES: COCCn1nc2c(c1C)/C=C/c1n[nH]c3ccc(cc13)-c1cnn(C)c1OCCCO2] | (17E)-15-(2-methoxyethyl)-8,16- dimethyl-2,11,12,15-tetrahydro-8H,10H- 3,5- etheno[1,5]dioxacyclopentadecino[10,11- c:15,14-c′:6,7-c″]tripyrazole |
| 73 | 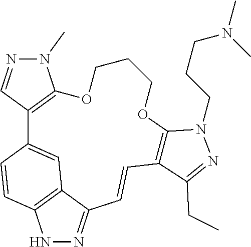 [SMILES: CCc1nn(CCCN(C)C)c2c1/C=C/c1n[nH]c3ccc(cc13)-c1cnn(C)c1OCCCO2] | 3-[(17E)-16-ethyl-8-methyl-2,8,11,12- tetrahydro-10H,14H-3,5- etheno[1,5]dioxacyclopentadecino[10,11- c:15,14-c′:6,7-c″]tripyrazol-14-yl]-N,N- dimethylpropan-1-amine |
| 74 | 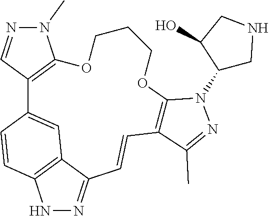 [SMILES: Cc1nn([C@H]2CNC[C@@H]2O)c2c1/C=C/c1n[nH]c3ccc(cc13)-c1cnn(C)c1OCCCO2] | (rac)-4-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14- yl]pyrrolidin-3-ol |
| 75 | 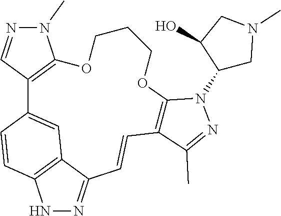 [SMILES: Cc1nn([C@H]2CN(C)C[C@@H]2O)c2c1/C=C/c1n[nH]c3ccc(cc13)-c1cnn(C)c1OCCCO2] | (rac)-4-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14-yl]-1- methylpyrrolidin-3-ol |
| 76 | 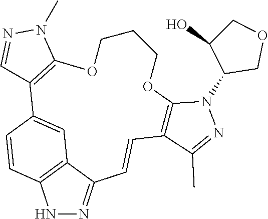 [SMILES: Cc1nn([C@H]2COC[C@@H]2O)c2c1/C=C/c1n[nH]c3ccc(cc13)-c1cnn(C)c1OCCCO2] | (3R,4S)-4-[(17E)-8,16-dimethyl- 2,8,11,12-tetrahydro-10H,14H-3,5- etheno[1,5]dioxacyclopentadecino[10,11- c:15,14-c′:6,7-c″]tripyrazol-14-yl]oxolan- 3-ol |
| 77 | 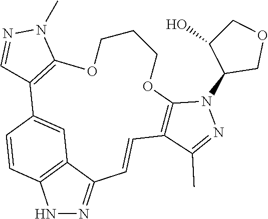 [SMILES: Cc1nn([C@@H]2COC[C@H]2O)c2c1/C=C/c1n[nH]c3ccc(cc13)-c1cnn(C)c1OCCCO2] | (3S,4R)-4-[(17E)-8,16-dimethyl- 2,8,11,12-tetrahydro-10H,14H-3,5- etheno[1,5]dioxacyclopentadecino[10,11- c:15,14-c′:6,7-c″]tripyrazol-14-yl]oxolan- 3-ol |
| 78 | 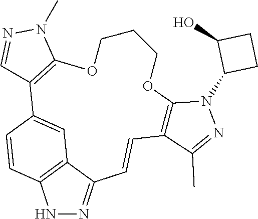 [SMILES: Cc1nn([C@H]2CC[C@@H]2O)c2c1/C=C/c1n[nH]c3ccc(cc13)-c1cnn(C)c1OCCCO2] | (rac)-2-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14- yl]cyclobutan-1-ol |
| 79 | 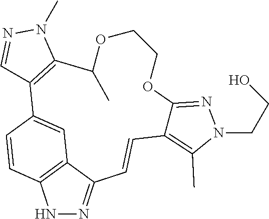 [SMILES: Cc1c2c(nn1CCO)OCCOC(C)c1c(cnn1C)-c1ccc3[nH]nc(c3c1)/C=C/2] | 2-[(17E)-8,9,16-trimethyl-8,9,11,12- tetrahydro-3,5- etheno[1,4]dioxacyclopentadecino[11,10- c:15,14-c′:6,7-c″]tripyrazol-15(2H)- yl]ethan-1-ol |
| 80 | 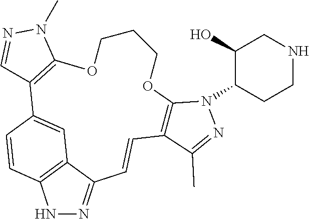 [SMILES: Cc1nn([C@H]2CCNC[C@@H]2O)c2c1/C=C/c1n[nH]c3ccc(cc13)-c1cnn(C)c1OCCCO2] | (rac)-4-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14- yl]piperidin-3-ol |
| 81 | 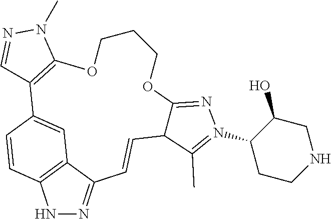 | (rac)-4-[(17E)-8,16-dimethyl-2,8,11,12- tetrahydro-10H,15H-3,5- etheno[1,5]dioxacyclopentadecino[10,11- c:15,14-c′:6,7-c″]tripyrazol-15- yl]piperidin-3-ol |
| 82 | 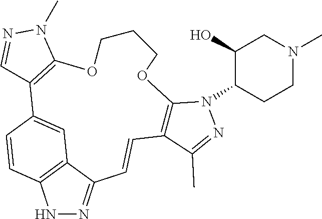 [SMILES: Cc1nn([C@H]2CCN(C)C[C@@H]2O)c2c1/C=C/c1n[nH]c3ccc(cc13)-c1cnn(C)c1OCCCO2] | (rac)-4-[(17E)-8,16-dimethyl-2,8,11,12- tetrahydro-10H,14H-3,5- etheno[1,5]dioxacyclopentadecino[10,11- c:15,14-c′:6,7-c″]tripyrazol-14-yl]-1- methylpiperidin-3-ol |
| 83 | 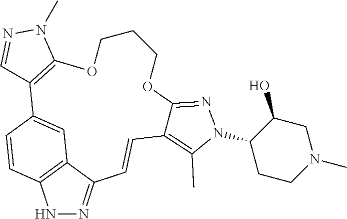 [SMILES: Cc1c2c(nn1[C@H]1CCN(C)C[C@@H]1O)OCCCOc1c(cnn1C)-c1ccc3[nH]nc(c3c1)/C=C/2] | (rac)-4-[(17E)-8,16-dimethyl-2,8,11,12- tetrahydro-10H,15H-3,5- etheno[1,5]dioxacyclopentadecino[10,11- c:15,14-c′:6,7-c″]tripyrazol-15-yl]-1- methylpiperidin-3-ol |
| 84 | 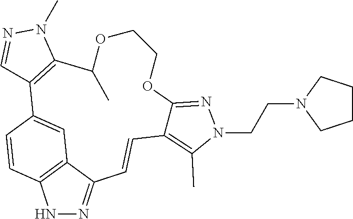 [SMILES: Cc1c2c(nn1CCN1CCCC1)OCCOC(C)c1c(cnn1C)-c1ccc3[nH]nc(c3c1)/C=C/2] | (17E)-8,9,16-trimethyl-15-[2-(pyrrolidin- 1-yl)ethyl]-2,8,9,11,12,15-hexahydro- 3,5- etheno[1,4]dioxacyclopentadecino[11,10- c:15,14-c′:6,7-c″]tripyrazole |
| 85 | 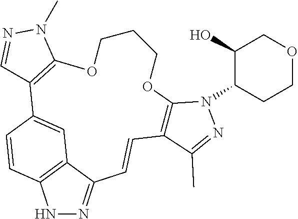 [SMILES: Cc1nn([C@H]2CCOC[C@@H]2O)c2c1/C=C/c1n[nH]c3ccc(cc13)-c1cnn(C)c1OCCCO2] | (rac)-1,5-anhydr-30-2,3-dideoxy-3- [(17E)-8,16-dimethyl-2,8,11,12- tetrahydro-10H,14H-3,5- etheno[1,5]dioxacyclopentadecino[10,11- c:15,14-c′:6,7-c″]tripyrazol-14-yl]-L- threo-pentitol |
| 86 | 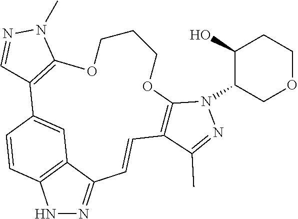 [SMILES: Cc1nn([C@H]2COCC[C@@H]2O)c2c1/C=C/c1n[nH]c3ccc(cc13)-c1cnn(C)c1OCCCO2] | (rac)-1,5-anhydro-2,4-dideoxy-2-[(17E)- 8,16-dimethyl-2,8,11,12-tetrahydro- 10H,14H-3,5- etheno[1,5]dioxacyclopentadecino[10,11- c:15,14-c′:6,7-c″]tripyrazol-14-yl]-L- threo-pentitol |
| 87 | 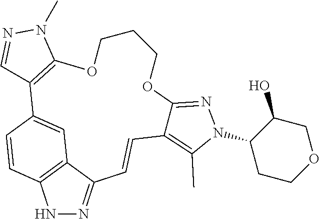 [SMILES: Cc1c2c(nn1[C@H]1CCOC[C@@H]1O)OCCCOc1c(cnn1C)-c1ccc3[nH]nc(c3c1)/C=C/2] | (rac)-1,5-anhydro-2,3-dideoxy-3-[(17E)- 8,16-dimethyl-2,8,11,12-tetrahydro- 10H,15H-3,5- etheno[1,5]dioxacyclopentadecino[10,11- c:15,14-c′:6,7-c″]tripyrazol-15-yl]-L- threo-pentitol |
| 88 | 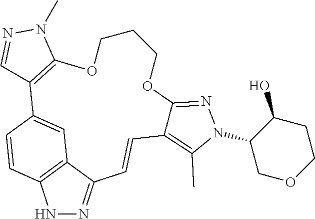 [SMILES: Cc1c2c(nn1[C@H]1COCC[C@@H]1O)OCCCOc1c(cnn1C)-c1ccc3[nH]nc(c3c1)/C=C/2] | 1,5-anhydro-2,4-dideoxy-2-[(17E)-8,16- dimethyl-2,8,11,12-tetrahydro-10H,15H- 3,5- etheno[1,5]dioxacyclopentadecino[10,11- c:15,14-c′:6,7-c″]tripyrazol-15-yl]-L- threo-pentitol |
| 89 | 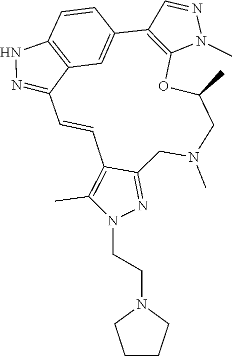 [SMILES: Cc1c2c(nn1CCN1CCCC1)CN(C)C[C@H](C)Oc1c(cnn1C)-c1ccc3[nH]nc(c3c1)/C=C/2] | (10S,17E)-8,10,12,16-tetramethyl-15-[2- (pyrrolidin-1-yl)ethyl]-2,10,11,12,13,15- hexahydro-8H-3,5-ethenotripyrazolo[3,4- f:3′,4′-j:4″,3″- n][1,4]oxazacyclopentadecine |
| 90 | 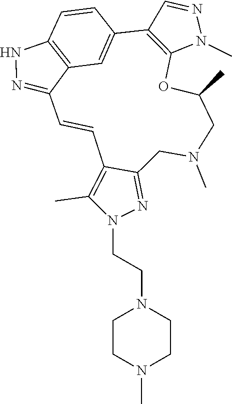 [SMILES: Cc1c2c(nn1CCN1CCN(C)CC1)CN(C)C[C@H](C)Oc1c(cnn1C)-c1ccc3[nH]nc(c3c1)/C=C/2] | (10S,17E)-8,10,12,16-tetramethyl-15-[2- (4-methylpiperazin-1-yl)ethyl]- 2,10,11,12,13,15-hexahydro-8H-3,5- ethenotripyrazolo[3,4-f:3′,4′-j:4″,3″- n][1,4]oxazacyclopentadecine |
| 91 | 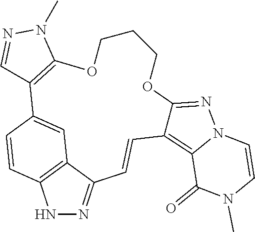 [SMILES: Cn1ncc2c1OCCCOc1nn3ccn(C)c(=O)c3c1/C=C/c1n[nH]c3ccc-2cc13] | (20E)-8,18-dimethyl-2,8,11,12- tetrahydro-10H-3,5- ethenodipyrazolo[3″,4″:10′,11′;4″,3′:14′, 15′][1,5]dioxacyclopentadecino[6′,7′:3,4] pyrazolo[1,5-a]pyrazin-19(18H)-one |
| 92 | 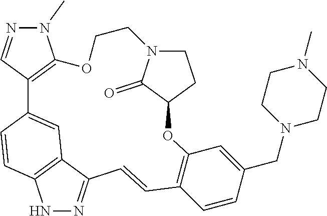 [SMILES: CN1CCN(Cc2ccc3c(c2)O[C@@H]2CCN(CCOc4c(cnn4C)-c4ccc5[nH]nc(c5c4)/C=C/3)C2=O)CC1] | (10R,16E)-3-methyl-13-[(4- methylpiperazin-1-yl)methyl]- 3,5,6,9,10,19-hexahydro-8H-20,22- etheno-7,10-methanodipyrazolo[3,4- i:4′,3′- m][1,8,5]benzodioxazacyclooctadecin- 25-one |
| 93 | 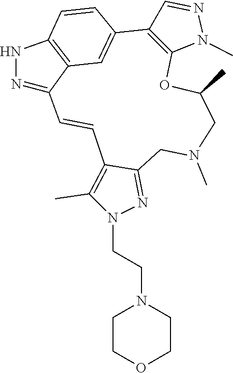 [SMILES: Cc1c2c(nn1CCN1CCOCC1)CN(C)C[C@H](C)Oc1c(cnn1C)-c1ccc3[nH]nc(c3c1)/C=C/2] | (10S,17E)-8,10,12,16-tetramethyl-15-[2- (morpholin-4-yl)ethyl]-2,10,11,12,13,15- hexahydro-8H-3,5-ethenotripyrazolo[3,4- f:3′,4′-j:4″,3″- n][1,4]oxazacyclopentadecine |
| 94 | 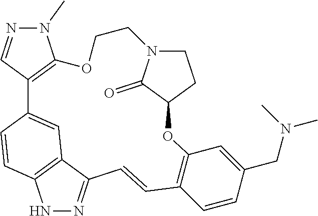 [SMILES: CN(C)Cc1ccc2c(c1)O[C@@H]1CCN(CCOc3c(cnn3C)-c3ccc4[nH]nc(c4c3)/C=C/2)C1=O] | (10R,16E)-13-[(dimethylamino)methyl]- 3-methyl-3,5,6,9,10,19-hexahydro-8H- 20,22-etheno-7,10- methanodipyrazolo[3,4-i:4′,3′- m][1,8,5]benzodioxazacyclooctadecin- 25-one |
| 95 | 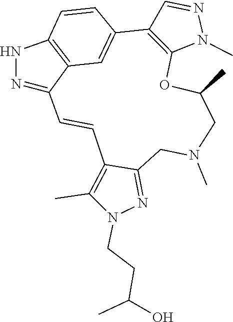 [SMILES: Cc1c2c(nn1CCC(C)O)CN(C)C[C@H](C)Oc1c(cnn1C)-c1ccc3[nH]nc(c3c1)/C=C/2] | 4-[(10S,17E)-8,10,12,16-tetramethyl- 2,8,10,11,12,13-hexahydro-15H-3,5- ethenotripyrazolo[3,4-f:3′,4′-j:4″,3″- n][1,4]oxazacyclopentadecin-15- yl]butan-2-ol |
| 96 | 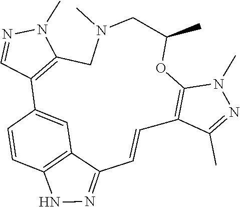 [SMILES: Cc1nn(C)c2c1/C=C/c1n[nH]c3ccc(cc13)-c1cnn(C)c1CN(C)C[C@@H](C)O2] | (12R,17E)-8,10,12,14,16-pentamethyl- 2,9,10,11,12,14-hexahydro-8H-3,5- ethenotripyrazolo[3,4-f:4′,3′-j:4″,3″- n[1,4]oxazacyclopentadecine |
| 97 | 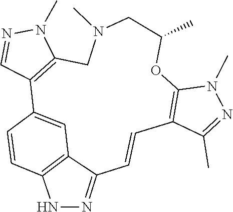 [SMILES: Cc1nn(C)c2c1/C=C/c1n[nH]c3ccc(cc13)-c1cnn(C)c1CN(C)C[C@H](C)O2] | (12S,17E)-8,10,12,14,16-pentamethyl- 2,9,10,11,12,14-hexahydro-8H-3,5- ethenotripyrazolo[3,4-f:4′,3′-j:4″,3″- n][1,4]oxazacyclopentadecine |
| 98 | 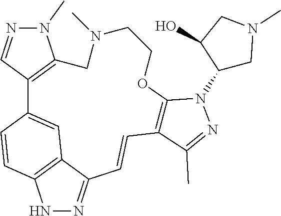 [SMILES: Cc1nn([C@H]2CN(C)C[C@@H]2O)c2c1/C=C/c1n[nH]c3ccc(cc13)-c1cnn(C)c1CN(C)CCO2] | (rac)-(3S,4S)-1-methyl-4-[(17E)-8,10,16- trimethyl-2,8,9,10,11,12-hexahydro-14H- 3,5-ethenotripyrazolo[3,4-f:4′,3′-j:4″,3″′- n][1,4]oxazacyclopentadecin-14- yl]pyrrolidin-3-ol |
| 99 | 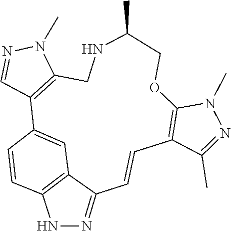 [SMILES: Cc1nn(C)c2c1/C=C/c1n[nH]c3ccc(cc13)-c1cnn(C)c1CN[C@@H](C)CO2] | (11S,17E)-8,11,14,16-tetramethyl- 2,9,10,11,12,14-hexahydro-8H-3,5- ethenotripyrazolo[3,4-f:4′,3′-j:4″,3″- n][1,4]oxazacyclopentadecine |
| 100 | 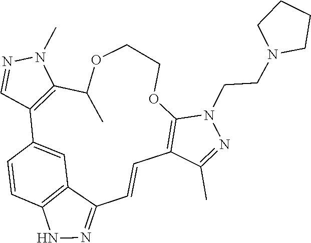 | (17E)-8,9,16-trimethyl-14-[2-(pyrrolidin- 1-yl)ethyl]-2,8,9,11,12,14-hexahydro- 3,5- etheno[1,4]dioxacyclopentadecino[11,10- c:15,14-c′:6,7-c″]tripyrazole |
| 101 | 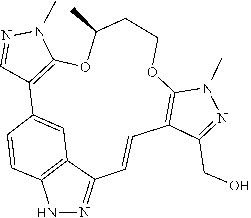 [SMILES: C[C@H]1CCOc2c(c(CO)nn2C)/C=C/c2n[nH]c3ccc(cc23)-c2cnn(C)c2O1] | [(10S,17E)-8,10,14-trimethyl-2,11,12,14- tetrahydro-8H,10H-3,5- etheno[1,5]dioxacyclopentadecino[10,11- c:15,14-c′:6,7-c″]tripyrazol-16- yl]methanol |
| 102 | 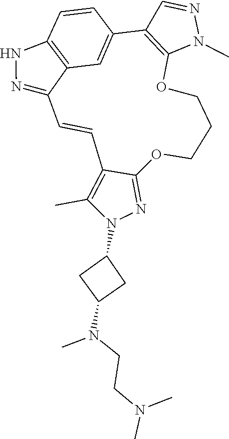 [SMILES: Cc1c2c(nn1[C@H]1C[C@@H](N(C)CCN(C)C)C1)OCCCOc1c(cnn1C)-c1ccc3[nH]nc(c3c1)/C=C/2] | N¹-{(1s,3s)-3-[(17E)-8,16-dimethyl- 2,8,11,12-tetrahydro-10H,15H-3,5- etheno[1,5]dioxacyclopentadecino[10,11- c:15,14-c′:6,7-c″]tripyrazol-15- yl]cyclobutyl}-N¹,N²,N²-trimethylethane- 1,2-diamine |
| 103 | 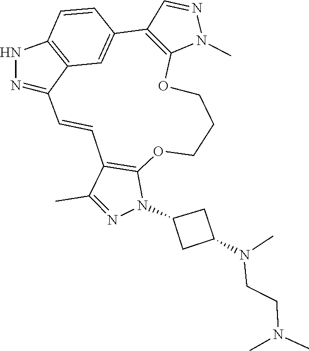 [SMILES: Cc1nn([C@H]2C[C@@H](N(C)CCN(C)C)C2)c2c1/C=C/c1n[nH]c3ccc(cc13)-c1cnn(C)c1OCCCO2] | N¹-{(1s,3s)-3-[(17E)-8,16-dimethyl- 2,8,11,12-tetrahydro-10H,14H-3,5- etheno[1,5]dioxacyclopentadecino[10,11- c:15,14-c′:6,7-c″]tripyrazol-14- yl]cyclobutyl}-N¹,N²,N²-trimethylethane- 1,2-diamine |
| 104 | 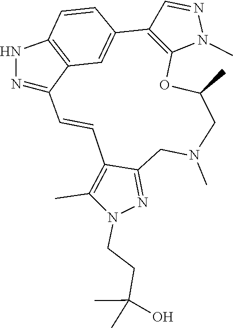 [SMILES: Cc1c2c(nn1CCC(C)(C)O)CN(C)C[C@H](C)Oc1c(cnn1C)-c1ccc3[nH]nc(c3c1)/C=C/2] | 2-methyl-4-[(10S,17E)-8,10,12,16- tetramethyl-2,8,10,11,12,13-hexahydro- 15H-3,5-ethenotripyrazolo[3,4-f:3′,4′- j:4″,3″-n][1,4]oxazacyclopentadecin-15- yl]butan-2-ol |
| 105 | 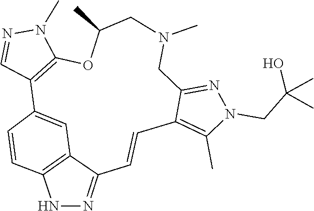 [SMILES: Cc1c2c(nn1CC(C)(C)O)CN(C)C[C@H](C)Oc1c(cnn1C)-c1ccc3[nH]nc(c3c1)/C=C/2] | 2-methyl-1-[(10S,17E)-8,10,12,16- tetramethyl-2,8,10,11,12,13-hexahydro- 15H-3,5-ethenotripyrazolo[3,4-f:3′,4′- j:4″,3″-n][1,4]oxazacyclopentadecin-15- yl]propan-2-ol |
| 106 | 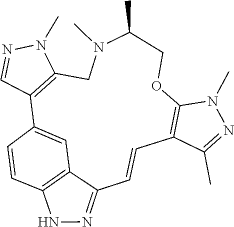 [SMILES: Cc1nn(C)c2c1/C=C/c1n[nH]c3ccc(cc13)-c1cnn(C)c1CN(C)[C@@H](C)CO2] | (11S,17E)-8,10,11,14,16-pentamethyl- 2,9,10,11,12,14-hexahydro-8H-3,5- ethenotripyrazolo[3,4-f:4′,3′-j:4″,3″- n][1,4]oxazacyclopentadecine |
| 107 | 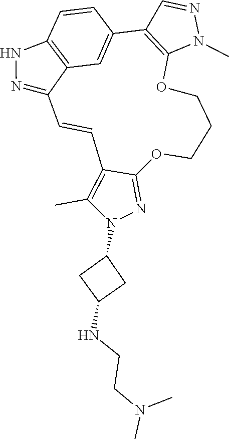 [SMILES: Cc1c2c(nn1[C@H]1C[C@@H](NCCN(C)C)C1)OCCCOc1c(cnn1C)-c1ccc3[nH]nc(c3c1)/C=C/2] | N²-{(1s,3s)-3-[(17E)-8,16-dimethyl- 2,8,11,12-tetrahydro-10H,15H-3,5- etheno[1,5]dioxacyclopentadecino[10,11- c:15,14-c′:6,7-c″]tripyrazol-15- yl]cyclobutyl}-N¹,N¹-dimethylethane- 1,2-diamine |
| 108 | 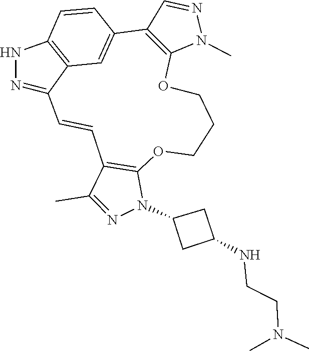 [SMILES: Cc1nn([C@H]2C[C@@H](NCCN(C)C)C2)c2c1/C=C/c1n[nH]c3ccc(cc13)-c1cnn(C)c1OCCCO2] | N²-{(1s,3s)-3-[(17E)-8,16-dimethyl- 2,8,11,12-tetrahydro-10H,14H-3,5- etheno[1,5]dioxacyclopentadecino[10,11- c:15,14-c′:6,7-c″]tripyrazol-14- yl]cyclobutyl}-N¹,N¹-dimethylethane- 1,2-diamine |
| 109 | 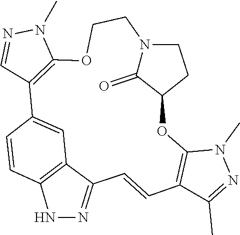 | (10R,15E)-3,12,14-trimethyl- 5,6,9,10,12,18-hexahydro-3H,8H-19,21- etheno-7,10-methanotripyrazolo[3,4- i:3′,4′-m:4″,3″- q][1,8,4]dioxazacyclooctadecin-24-one |
| 110 | 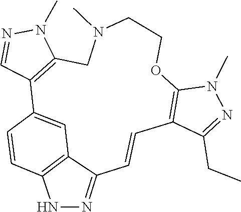 [SMILES: CCc1nn(C)c2c1/C=C/c1n[nH]c3ccc(cc13)-c1cnn(C)c1CN(C)CCO2] | (17E)-16-ethyl-8,10,14-trimethyl- 2,9,10,11,12,14-hexahydro-8H-3,5- ethenotripyrazolo[3,4-f:4′,3′-j:4″,3″- n][1,4]oxazacyclopentadecine |
| 111 | 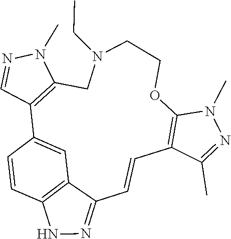 [SMILES: CCN1CCOc2c(c(C)nn2C)/C=C/c2n[nH]c3ccc(cc23)-c2cnn(C)c2C1] | (17E)-10-ethyl-8,14,16-trimethyl- 2,9,10,11,12,14-hexahydro-8H-3,5- ethenotripyrazolo[3,4-f:4′,3′-j:4″,3″- n][1,4]oxazacyclopentadecine |
| 112 | 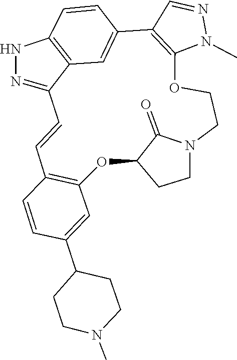 [SMILES: CN1CCC(c2ccc3c(c2)O[C@@H]2CCN(CCOc4c(cnn4C)-c4ccc5[nH]nc(c5c4)/C=C/3)C2=O)CC1] | (10R,16E)-3-methyl-13-(1- methylpiperidin-4-yl)-3,5,6,9,10,19- hexahydro-8H-20,22-etheno-7,10- methanodipyrazolo[3,4-i:4′,3′- m][1,8,5]benzodioxazacyclooctadecin- 25-one |
| 113 | 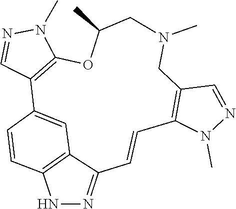 [SMILES: C[C@H]1CN(C)Cc2cnn(C)c2/C=C/c2n[nH]c3ccc(cc23)-c2cnn(C)c2O1] | (10S,17E)-8,10,12,16-tetramethyl- 2,10,11,12,13,16-hexahydro-8H-3,5- ethenotripyrazolo[4,3-f:3′,4′-j:4″,3″- n][1,4]oxazacyclopentadecine |
| 114 | 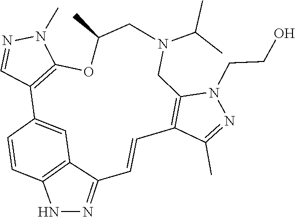 | 2-[(10S,17E)-8,10,16-trimethyl-12- (propan-2-yl)-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 115 | 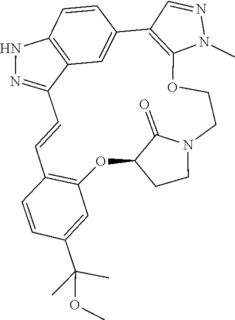 [SMILES: COC(C)(C)c1ccc2c(c1)O[C@@H]1CCN(CCOc3c(cnn3C)-c3ccc4[nH]nc(c4c3)/C=C/2)C1=O] | (10R,16E)-13-(2-methoxypropan-2-yl)-3- methyl-3,5,6,9,10,19-hexahydro-8H- 20,22-etheno-7,10- methanodipyrazolo[3,4-i:4′,3′- m][1,8,5]benzodioxazacyclooctadecin- 25-one |
| 116 | 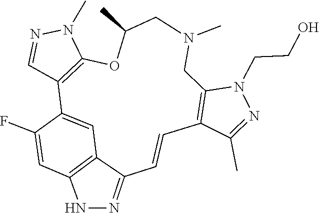 [SMILES: Cc1nn(CCO)c2c1/C=C/c1n[nH]c3cc(F)c(cc13)-c1cnn(C)c1O[C@@H](C)CN(C)C2] | 2-[(10S,17E)-19-fluoro-8,10,12,16- tetramethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 117 | 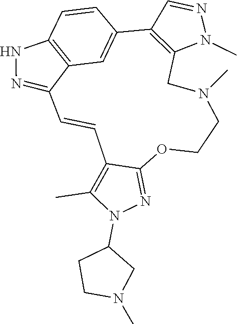 [SMILES: Cc1c2c(nn1C1CCN(C)C1)OCCN(C)Cc1c(cnn1C)-c1ccc3[nH]nc(c3c1)/C=C/2] | (17E)-8,10,16-trimethyl-15-(1- methylpyrrolidin-3-yl)-2,9,10,11,12,15- hexahydro-8H-3,5-ethenotripyrazolo[3,4- f:4′,3′-j:4″,3″- n][1,4]oxazacyclopentadecine |
| 118 | 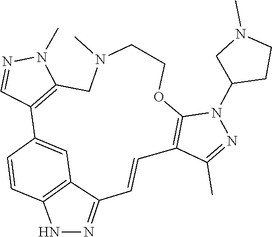 [SMILES: Cc1nn(C2CCN(C)C2)c2c1/C=C/c1n[nH]c3ccc(cc13)-c1cnn(C)c1CN(C)CCO2] | (17E)-8,10,16-trimethyl-14-(1- methylpyrrolidin-3-yl)-2,9,10,11,12,14- hexahydro-8H-3,5-ethenotripyrazolo[3,4- f:4′,3′-j:4″,3″- n][1,4]oxazacyclopentadecine |
| 119 | 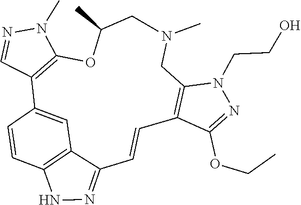 | 2-[(10S,17E)-16-ethoxy-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 120 | 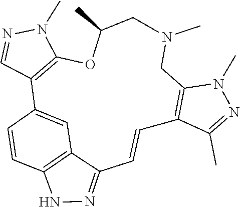 [SMILES: Cc1nn(C)c2c1/C=C/c1n[nH]c3ccc(cc13)-c1cnn(C)c1O[C@@H](C)CN(C)C2] | (10S,17E)-8,10,12,14,16-pentamethyl- 2,10,11,12,13,14-hexahydro-8H-3,5- ethenotripyrazolo[3,4-f:3′,4′-j:4″,3″- n][1,4]oxazacyclopentadecine |
| 121 | 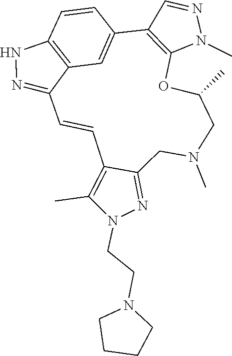 [SMILES: Cc1c2c(nn1CCN1CCCC1)CN(C)C[C@@H](C)Oc1c(cnn1C)-c1ccc3[nH]nc(c3c1)/C=C/2] | (10R,17E)-8,10,12,16-tetramethyl-15-[2- (pyrrolidin-1-yl)ethyl]-2,10,11,12,13,15- hexahydro-8H-3,5-ethenotripyrazolo[3,4- f:3′,4′-j:4″,3″- n][1,4]oxazacyclopentadecine |
| 122 | 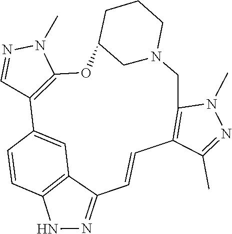 [SMILES: Cc1nn(C)c2c1/C=C/c1n[nH]c3ccc(cc13)-c1cnn(C)c1O[C@@H]1CCCN(C2)C1] | (10R,19E)-8,16,18-trimethyl- 2,8,10,11,12,13,15,16-octahydro-3,5- etheno-10,14-methanotripyrazolo[3,4- h:3′,4′-l:4″,3″- p][1,6]oxazacycloheptadecine |
| 123 | 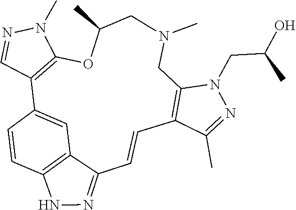 [SMILES: Cc1nn(C[C@H](C)O)c2c1/C=C/c1n[nH]c3ccc(cc13)-c1cnn(C)c1O[C@@H](C)CN(C)C2] | (2S)-1-[(10S,17E)-8,10,12,16- tetramethyl-2,8,10,11,12,13-hexahydro- 14H-3,5-ethenotripyrazolo[3,4-f:3′,4′- j:4″,3″-n][1,4]oxazacyclopentadecin-14- yl]propan-2-ol |
| 124 | 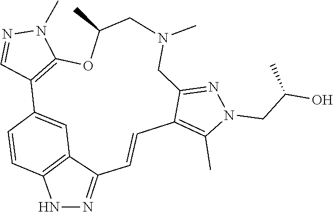 [SMILES: Cc1c2c(nn1C[C@H](C)O)CN(C)C[C@H](C)Oc1c(cnn1C)-c1ccc3[nH]nc(c3c1)/C=C/2] | (2S)-1-[(10S,17E)-8,10,12,16- tetramethyl-2,8,10,11,12,13-hexahydro- 15H-3,5-ethenotripyrazolo[3,4-f:3′,4′- j:4″,3″-n][1,4]oxazacyclopentadecin-15- yl]propan-2-ol |
| 125 | 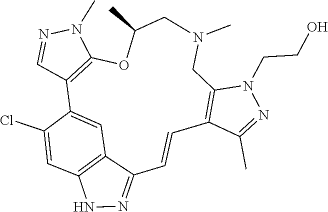 [SMILES: Cc1nn(CCO)c2c1/C=C/c1n[nH]c3cc(Cl)c(cc13)-c1cnn(C)c1O[C@@H](C)CN(C)C2] | 2-[(10S,17E)-19-chloro-8,10,12,16- tetramethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 126 | 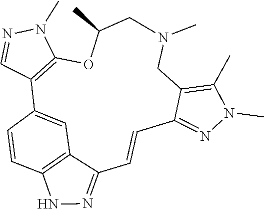 [SMILES: Cc1c2c(nn1C)/C=C/c1n[nH]c3ccc(cc13)-c1cnn(C)c1O[C@@H](C)CN(C)C2] | (10S,17E)-8,10,12,14,15-pentamethyl- 2,10,11,12,13,15-hexahydro-8H-3,5- ethenotripyrazolo[4,3-f:3′,4′-j:4″,3″- n][1,4]oxazacyclopentadecine |
| 127 | 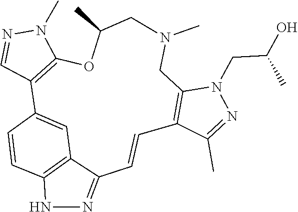 | (2R)-1-[(10S,17E)-8,10,12,16- tetramethyl-2,8,10,11,12,13-hexahydro- 14H-3,5-ethenotripyrazolo[3,4-f:3′,4′- j:4″,3″-n][1,4]oxazacyclopentadecin-14- yl]propan-2-ol |
| 128 | 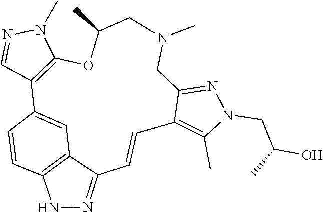 | (2R)-1-[(10S,17E)-8,10,12,16- tetramethyl-2,8,10,11,12,13-hexahydro- 15H-3,5-ethenotripyrazolo[3,4-f:3′,4′- j:4″,3″-n][1,4]oxazacyclopentadecin-15- yl]propan-2-ol |
| 129 | 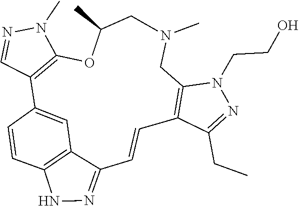 [SMILES: CCc1nn(CCO)c2c1/C=C/c1n[nH]c3ccc(cc13)-c1cnn(C)c1O[C@@H](C)CN(C)C2] | 2-[(10S,17E)-16-ethyl-8,10,12-trimethyl- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 130 | 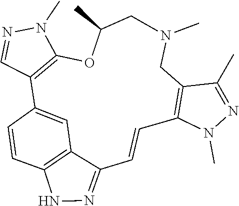 [SMILES: Cc1nn(C)c2c1CN(C)C[C@H](C)Oc1c(cnn1C)-c1ccc3[nH]nc(c3c1)/C=C/2] | (10S,17E)-8,10,12,14,16-pentamethyl- 2,10,11,12,13,16-hexahydro-8H-3,5- ethenotripyrazolo[4,3-f:3′,4′-j:4″,3″- n][1,4]oxazacyclopentadecine |
| 131 | 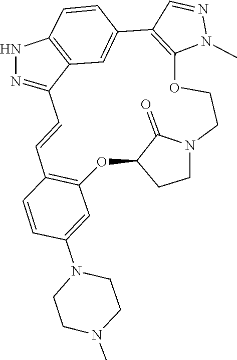 [SMILES: CN1CCN(c2ccc3c(c2)O[C@@H]2CCN(CCOc4c(cnn4C)-c4ccc5[nH]nc(c5c4)/C=C/3)C2=O)CC1] | (10R,16E)-3-methyl-13-(4- methylpiperazin-1-yl)-3,5,6,9,10,19- hexahydro-8H-20,22-etheno-7,10- methanodipyrazolo[3,4-i:4′,3′- m][1,8,5]benzodioxazacyclooctadecin- 25-one |
| 132 | 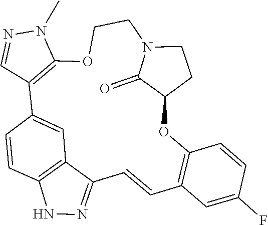 [SMILES: Cn1ncc2c1OCCN1CC[C@@H](Oc3ccc(F)cc3/C=C/c3n[nH]c4ccc-2cc34)C1=O] | (10R,16E)-14-fluoro-3-methyl- 3,5,6,9,10,19-hexahydro-8H-20,22- etheno-7,10-methanodipyrazolo[3,4- i:4′,3′- m][1,8,5]benzodioxazacyclooctadecin- 25-one |
| 133 | 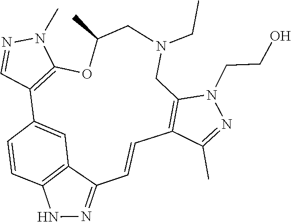 [SMILES: CCN1Cc2c(c(C)nn2CCO)/C=C/c2n[nH]c3ccc(cc23)-c2cnn(C)c2O[C@@H](C)C1] | 2-[(10S,17E)-12-ethyl-8,10,16-trimethyl- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 134 | 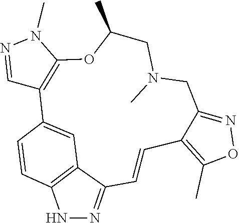 [SMILES: Cc1onc2c1/C=C/c1n[nH]c3ccc(cc13)-c1cnn(C)c1O[C@@H](C)CN(C)C2] | (10S,17E)-8,10,12,16-tetramethyl- 2,8,10,11,12,13-hexahydro-3,5- etheno[1,2]oxazolo[3,4-f]dipyrazolo[3,4- j:4′,3′-n][1,4]oxazacyclopentadecine |
| 135 | 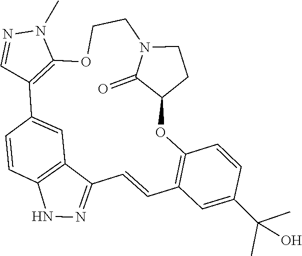 [SMILES: Cn1ncc2c1OCCN1CC[C@@H](Oc3ccc(C(C)(C)O)cc3/C=C/c3n[nH]c4ccc-2cc34)C1=O] | (10R,16E)-14-(2-hydroxypropan-2-yl)-3- methyl-3,5,6,9,10,19-hexahydro-8H- 20,22-etheno-7,10- methanodipyrazolo[3,4-i:4′,3′- m][1,8,5]benzodioxazacyclooctadecin- 25-one |
| 136 | 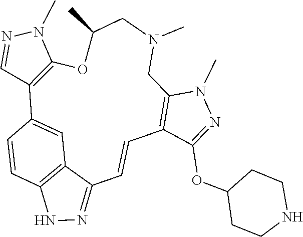 | (10S,17E)-8,10,12,14-tetramethyl-16- [(piperidin-4-yl)oxy]-2,10,11,12,13,14- hexahydro-8H-3,5-ethenotripyrazolo[3,4- f:3′,4′-j:4″,3″- n][1,4]oxazacyclopentadecine |
| 137 | 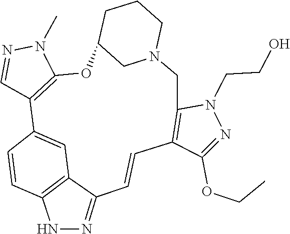 [SMILES: CCOc1nn(CCO)c2c1/C=C/c1n[nH]c3ccc(cc13)-c1cnn(C)c1O[C@@H]1CCCN(C2)C1] | 2-[(10R,19E)-18-ethoxy-8-methyl- 2,10,11,12,13,15-hexahydro-3,5-etheno- 10,14-methanotripyrazolo[3,4-h:3′,4′- l:4″,3″-p][1,6]oxazacycloheptadecin- 16(8H)-yl]ethan-1-ol |
| 138 | 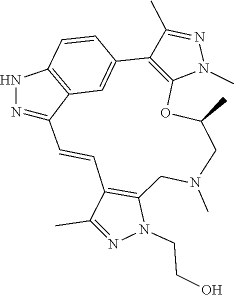 [SMILES: Cc1nn(CCO)c2c1/C=C/c1n[nH]c3ccc(cc13)-c1c(C)nn(C)c1O[C@@H](C)CN(C)C2] | 2-[(10S,17E)-6,8,10,12,16-pentamethyl- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 139 | 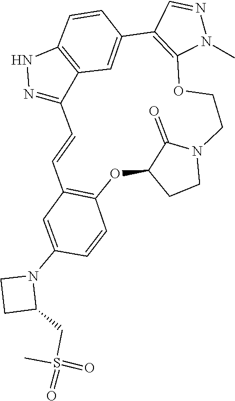 [SMILES: Cn1ncc2c1OCCN1CC[C@@H](Oc3ccc(N4CC[C@H]4CS(C)(=O)=O)cc3/C=C/c3n[nH]c4ccc-2cc34)C1=O] | (10R,16E)-14-{(25)-2- [(methanesulfonyl)methyl]azetidin-1-yl}- 3-methyl-3,5,6,9,10,19-hexahydro-8H- 20,22-etheno-7,10- methanodipyrazolo[3,4-i:4′,3′- m][1,8,5]benzodioxazacyclooctadecin- 25-one |
| 140 | 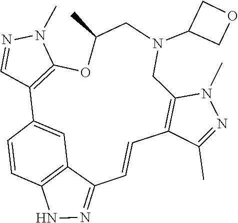 [SMILES: Cc1nn(C)c2c1/C=C/c1n[nH]c3ccc(cc13)-c1cnn(C)c1O[C@@H](C)CN(C1COC1)C2] | (10S,17E)-8,10,14,16-tetramethyl-12- (oxetan-3-yl)-2,10,11,12,13,14- hexahydro-8H-3,5-ethenotripyrazolo[3,4- f:3′,4′-j:4″,3″- n][1,4]oxazacyclopentadecine |
| 141 | 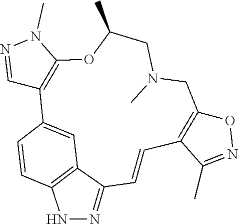 [SMILES: Cc1noc2c1/C=C/c1n[nH]c3ccc(cc13)-c1cnn(C)c1O[C@@H](C)CN(C)C2] | (10S,17E)-8,10,12,16-tetramethyl- 2,8,10,11,12,13-hexahydro-3,5- etheno[1,2]oxazolo[5,4-f]dipyrazolo[3,4- j:4′,3′-n][1,4]oxazacyclopentadecine |
| 142 | 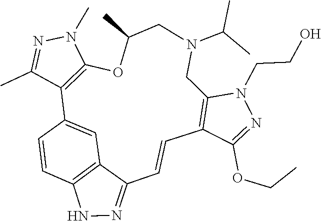 | 2-[(10S,17E)-16-ethoxy-6,8,10-trimethyl- 12-(propan-2-yl)-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 143 | 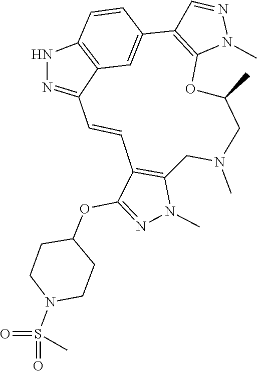 [SMILES: C[C@H]1CN(C)Cc2c(c(OC3CCN(S(C)(=O)=O)CC3)nn2C)/C=C/c2n[nH]c3ccc(cc23)-c2cnn(C)c2O1] | (10S,17E)-16-{[1- (methanesulfonyl)piperidin-4-yl]oxy}- 8,10,12,14-tetramethyl-2,10,11,12,13,14- hexahydro-8H-3,5-ethenotripyrazolo[3,4- f:3′,4′-j:4″,3″- n][1,4]oxazacyclopentadecine |
| 144 | 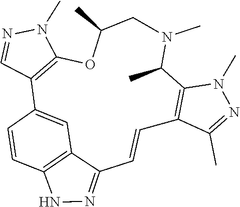 [SMILES: Cc1nn(C)c2c1/C=C/c1n[nH]c3ccc(cc13)-c1cnn(C)c1O[C@@H](C)CN(C)[C@@H]2C] | (10S,13R,17E)-8,10,12,13,14,16- hexamethyl-2,10,11,12,13,14-hexahydro- 8H-3,5-ethenotripyrazolo[3,4-f:3′,4′- j:4″,3″-n][1,4]oxazacyclopentadecine |
| 145 | 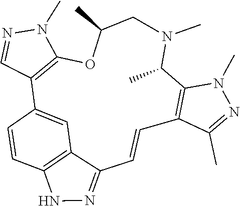 [SMILES: Cc1nn(C)c2c1/C=C/c1n[nH]c3ccc(cc13)-c1cnn(C)c1O[C@@H](C)CN(C)[C@H]2C] | (10S,13S,17E)-8,10,12,13,14,16- hexamethyl-2,10,11,12,13,14-hexahydro- 8H-3,5-ethenotripyrazolo[3,4-f:3′,4′- j:4″,3″-n][1,4]oxazacyclopentadecine |
| 146 | 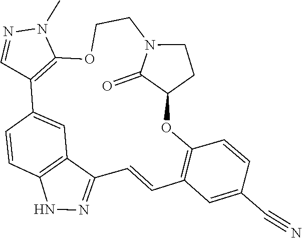 [SMILES: Cn1ncc2c1OCCN1CC[C@@H](Oc3ccc(C#N)cc3/C=C/c3n[nH]c4ccc-2cc34)C1=O] | (10R,16E)-3-methyl-25-oxo- 3,5,6,9,10,19-hexahydro-8H-20,22- etheno-7,10-methanodipyrazolo[3,4- i:4′,3′- m][1,8,5]benzodioxazacyclooctadecine- 14-carbonitrile |
| 147 | 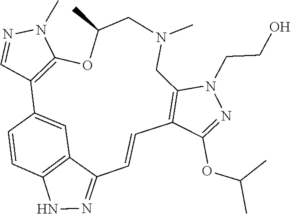 | 2-{(10S,17E)-8,10,12-trimethyl-16- [(propan-2-yl)oxy]-2,8,10,11,12,13- hexahydro-14H-3,5- ethenotripyrazolo[3,4-f:3′,4′-j:4″,3″- n][1,4]oxazacyclopentadecin-14- yl}ethan-1-ol |
| 148 | 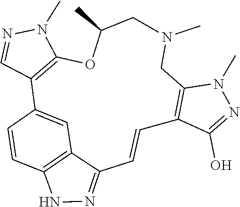 [SMILES: C[C@H]1CN(C)Cc2c(c(O)nn2C)/C=C/c2n[nH]c3ccc(cc23)-c2cnn(C)c2O1] | (10S,17E)-8,10,12,14-tetramethyl- 2,10,11,12,13,14-hexahydro-8H-3,5- ethenotripyrazolo[3,4-f:3′,4′-j:4″,3″- n][1,4]oxazacyclopentadecin-16-ol |
| 149 | 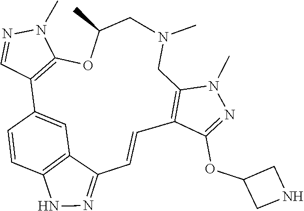 | (10S,17E)-16-[(azetidin-3-yl)oxy]- 8,10,12,14-tetramethyl-2,10,11,12,13,14- hexahydro-8H-3,5-ethenotripyrazolo[3,4- f:3′,4′-j:4″,3″- n][1,4]oxazacyclopentadecine |
| 150 | 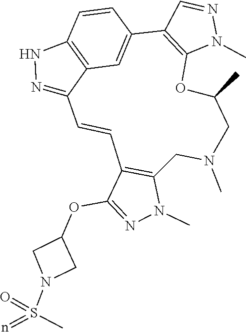 | (10S,17E)-16-{[1- (methanesulfonyl)azetidin-3-yl]oxy}- 8,10,12,14-tetramethyl-2,10,11,12,13,14- hexahydro-8H-3,5-ethenotripyrazolo[3,4- f:3′,4′-j:4″,3″- n][1,4]oxazacyclopentadecine |
| 151 | 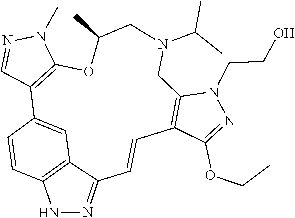 [SMILES: CCOc1nn(CCO)c2c1/C=C/c1n[nH]c3ccc(cc13)-c1cnn(C)c1O[C@@H](C)CN(C(C)C)C2] | 2-[(10S,17E)-16-ethoxy-8,10-dimethyl- 12-(propan-2-yl)-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 152 | 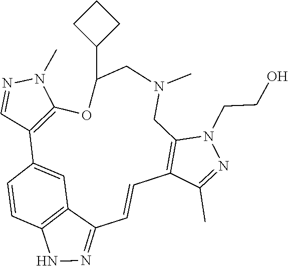 [SMILES: Cc1nn(CCO)c2c1/C=C/c1n[nH]c3ccc(cc13)-c1cnn(C)c1OC(C1CCC1)CN(C)C2] | 2-[(17E)-10-cyclobutyl-8,12,16- trimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 153 | 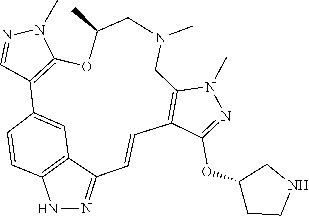 [SMILES: C[C@H]1CN(C)Cc2c(c(O[C@H]3CCNC3)nn2C)/C=C/c2n[nH]c3ccc(cc23)-c2cnn(C)c2O1] | (10S,17E)-8,10,12,14-tetramethyl-16- {[(3S)-pyrrolidin-3-yl]oxy}- 2,10,11,12,13,14-hexahydro-8H-3,5- ethenotripyrazolo[3,4-f:3′,4′-j:4″,3″- n][1,4]oxazacyclopentadecine |
| 154 | 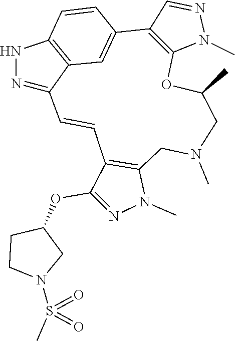 [SMILES: C[C@H]1CN(C)Cc2c(c(O[C@H]3CCN(S(C)(=O)=O)C3)nn2C)/C=C/c2n[nH]c3ccc(cc23)-c2cnn(C)c2O1] | (10S,17E)-16-{[(3S)-1- (methanesulfonyl)pyrrolidin-3-yl]oxy}- 8,10,12,14-tetramethyl-2,10,11,12,13,14- hexahydro-8H-3,5-ethenotripyrazolo[3,4- f:3′,4′-j:4″,3″- n][1,4]oxazacyclopentadecine |
| 155 | 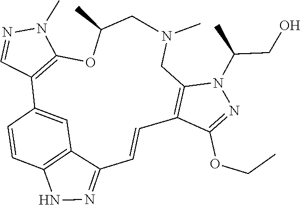 | (2S)-2-[(10S,17E)-16-ethoxy-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl]propan-1-ol |
| 156 | 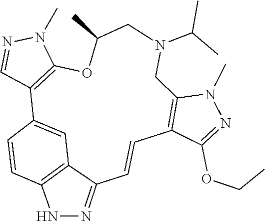 [SMILES: CCOc1nn(C)c2c1/C=C/c1n[nH]c3ccc(cc13)-c1cnn(C)c1O[C@@H](C)CN(C(C)C)C2] | (10S,17E)-16-ethoxy-8,10,14-trimethyl- 12-(propan-2-yl)-2,10,11,12,13,14- hexahydro-8H-3,5-ethenotripyrazolo[3,4- f:3′,4′-j:4″,3″- n][1,4]oxazacyclopentadecine |
| 157 | 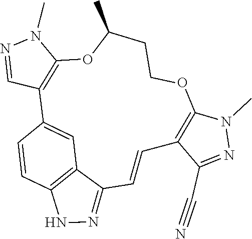 [SMILES: C[C@H]1CCOc2c(c(C#N)nn2C)/C=C/c2n[nH]c3ccc(cc23)-c2cnn(C)c2O1] | (10S,17E)-8,10,14-trimethyl-2,11,12,14- tetrahydro-8H,10H-3,5- etheno[1,5]dioxacyclopentadecino[10,11- c:15,14-c′:6,7-c″]tripyrazole-16- carbonitrile |
| 158 | 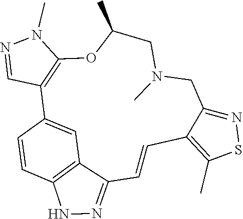 [SMILES: Cc1snc2c1/C=C/c1n[nH]c3ccc(cc13)-c1cnn(C)c1O[C@@H](C)CN(C)C2] | (10S,17E)-8,10,12,16-tetramethyl- 2,8,10,11,12,13-hexahydro-3,5- ethenodipyrazolo[3,4-j:4′,3′- n][1,2]thiazolo[3,4- f][1,4]oxazacyclopentadecine |
| 159 | 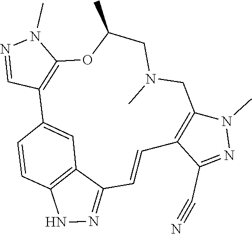 [SMILES: C[C@H]1CN(C)Cc2c(c(C#N)nn2C)/C=C/c2n[nH]c3ccc(cc23)-c2cnn(C)c2O1] | (10S,17E)-8,10,12,14-tetramethyl- 2,10,11,12,13,14-hexahydro-8H-3,5- ethenotripyrazolo[3,4-f:3′,4′-j:4″,3″- n][1,4]oxazacyclopentadecine-16- carbonitrile |
| 160 | 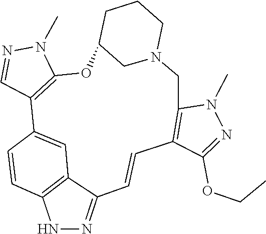 | (10R,19E)-18-ethoxy-8,16-dimethyl- 2,8,10,11,12,13,15,16-octahydro-3,5- etheno-10,14-methanotripyrazolo[3,4- h:3′,4′-l:4″,3″- p][1,6]oxazacycloheptadecine |
| 161 | 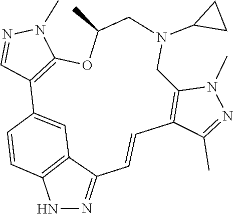 [SMILES: Cc1nn(C)c2c1/C=C/c1n[nH]c3ccc(cc13)-c1cnn(C)c1O[C@@H](C)CN(C1CC1)C2] | (10S,17E)-12-cyclopropyl-8,10,14,16- tetramethyl-2,10,11,12,13,14-hexahydro- 8H-3,5-ethenotripyrazolo[3,4-f:3′,4′- j:4″,3″-n][1,4]oxazacyclopentadecine |
| 162 | 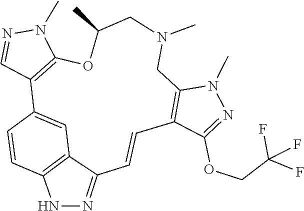 | (10S,17E)-8,10,12,14-tetramethyl-16- (2,2,2-trifluoroethoxy)-2,10,11,12,13,14- hexahydro-8H-3,5-ethenotripyrazolo[3,4- f:3′,4′-j:4″,3″- n][1,4]oxazacyclopentadecine |
| 163 | 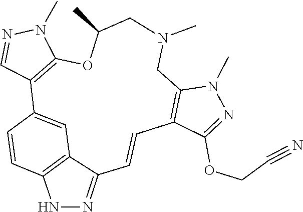 [SMILES: C[C@H]1CN(C)Cc2c(c(OCC#N)nn2C)/C=C/c2n[nH]c3ccc(cc23)-c2cnn(C)c2O1] | {[(10S,17E)-8,10,12,14-tetramethyl- 2,10,11,12,13,14-hexahydro-8H-3,5- ethenotripyrazolo[3,4-f:3′,4′-j:4″,3″- n][1,4]oxazacyclopentadecin-16- yl]oxy}acetonitrile |
| 164 | 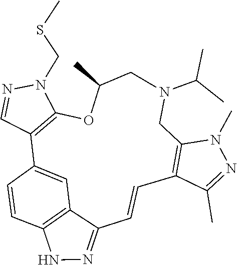 [SMILES: CSCn1ncc2c1O[C@@H](C)CN(C(C)C)Cc1c(c(C)nn1C)/C=C/c1n[nH]c3ccc-2cc13] | (10S,17E)-10,14,16-trimethyl-8- [(methylsulfanyl)methyl]-12-(propan-2- yl)-2,10,11,12,13,14-hexahydro-8H-3,5- ethenotripyrazolo[3,4-f:3′,4′-j:4″,3″- n\|[1,4]oxazacyclopentadecine |
| 165 | 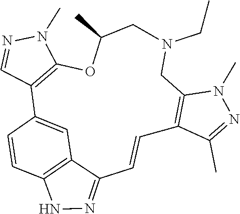 [SMILES: CCN1Cc2c(c(C)nn2C)/C=C/c2n[nH]c3ccc(cc23)-c2cnn(C)c2O[C@@H](C)C1] | (10S,17E)-12-ethyl-8,10,14,16- tetramethyl-2,10,11,12,13,14-hexahydro- 8H-3,5-ethenotripyrazolo[3,4-f:3′,4′- j:4″,3″-n][1,4]oxazacyclopentadecine |
| 166 | 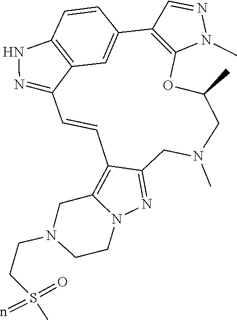 | (10S,20E)-18-[2- (methanesulfonyl)ethyl]-8,10,12- trimethyl-2,8,10,11,12,13,16,17,18,19- decahydro-3,5- ethenopyrazino[1′,2′:1,5]pyrazolo[3,4- f]dipyrazolo[3,4-j:4′,3′- |
| 167 | 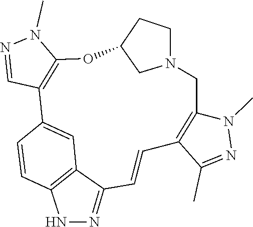 [SMILES: Cc1nn(C)c2c1/C=C/c1n[nH]c3ccc(cc13)-c1cnn(C)c1O[C@@H]1CCN(C2)C1] | n][1,4]oxazacyclopentadecine (10R,18E)-8,15,17-trimethyl- 2,8,11,12,14,15-hexahydro-10H-3,5- etheno-10,13-methanotripyrazolo[3,4- g:3′,4′-k:4″,3″- 0][1,5]oxazacyclohexadecine |
| 168 | 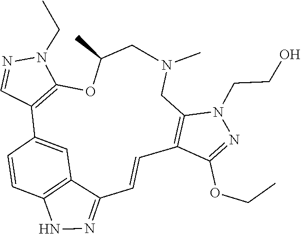 | 2-[(10S,17E)-16-ethoxy-8-ethyl-10,12- dimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 169 | 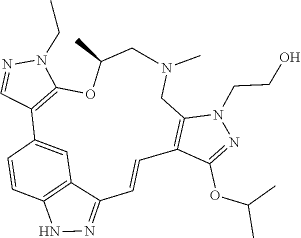 | 2-{(10S,17E)-8-ethyl-10,12-dimethyl-16- [(propan-2-yl)oxy]-2,8,10,11,12,13- hexahydro-14H-3,5- ethenotripyrazolo[3,4-f:3′,4′-j:4″,3″- n][1,4]oxazacyclopentadecin-14- yl}ethan-1-ol |
| 170 | 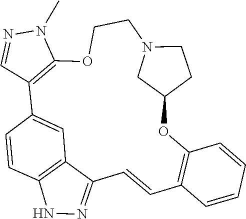 [SMILES: Cn1ncc2c1OCCN1CC[C@H](C1)Oc1ccccc1/C=C/c1n[nH]c3ccc-2cc13] | (10R,16E)-3-methyl-3,5,6,9,10,19- hexahydro-8H-20,22-etheno-7,10- methanodipyrazolo[3,4-i:4′,3′-m] [1,8,5]benzodioxazacyclooctadecine |
| 171 | 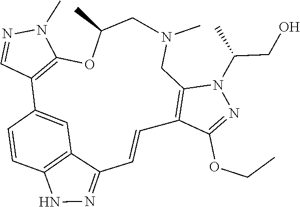 [SMILES: CCOc1nn([C@H](C)CO)c2c1/C=C/c1n[nH]c3ccc(cc13)-c1cnn(C)c1O[C@@H](C)CN(C)C2] | (2R)-2-[(10S,17E)-16-ethoxy-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl]propan-1-ol |
| 172 | 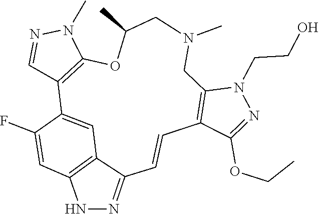 [SMILES: CCOc1nn(CCO)c2c1/C=C/c1n[nH]c3cc(F)c(cc13)-c1cnn(C)c1O[C@@H](C)CN(C)C2] | 2-[(10S,17E)-16-ethoxy-19-fluoro- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 173 | 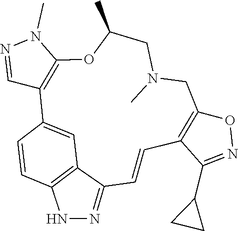 [SMILES: C[C@H]1CN(C)Cc2onc(C3CC3)c2/C=C/c2n[nH]c3ccc(cc23)-c2cnn(C)c2O1] | (10S,17E)-16-cyclopropyl-8,10,12- trimethyl-2,8,10,11,12,13-hexahydro-3,5- etheno[1,2]oxazolo[5,4-f]dipyrazolo[3,4- j:4′,3′-n][1,4]oxazacyclopentadecine |
| 174 | 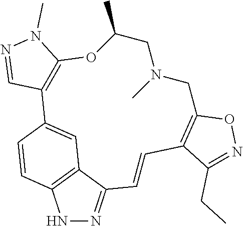 [SMILES: CCc1noc2c1/C=C/c1n[nH]c3ccc(cc13)-c1cnn(C)c1O[C@@H](C)CN(C)C2] | (10S,17E)-16-ethyl-8,10,12-trimethyl- 2,8,10,11,12,13-hexahydro-3,5- etheno[1,2]oxazolo[5,4-f]dipyrazolo[3,4- j:4′,3′-n][1,4]oxazacyclopentadecine |
| 175 | 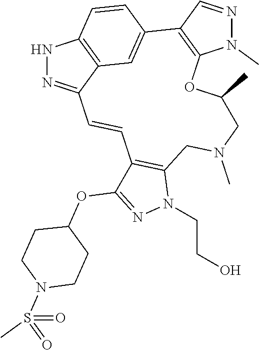 | 2-[(10S,17E)-16-{[1- (methanesulfonyl)piperidin-4-yl]oxy}- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 176 | 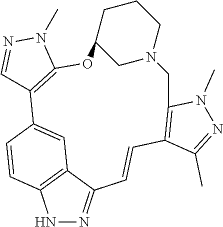 [SMILES: Cc1nn(C)c2c1/C=C/c1n[nH]c3ccc(cc13)-c1cnn(C)c1O[C@H]1CCCN(C2)C1] | (10S,19E)-8,16,18-trimethyl- 2,8,10,11,12,13,15,16-octahydro-3,5- etheno-10,14-methanotripyrazolo[3,4- h:3′,4′-l:4″,3″- p][1,6]oxazacycloheptadecine |
| 177 | 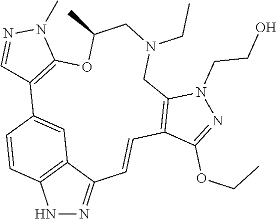 [SMILES: CCOc1nn(CCO)c2c1/C=C/c1n[nH]c3ccc(cc13)-c1cnn(C)c1O[C@@H](C)CN(CC)C2] | 2-[(10S,17E)-16-ethoxy-12-ethyl-8,10- dimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 178 | 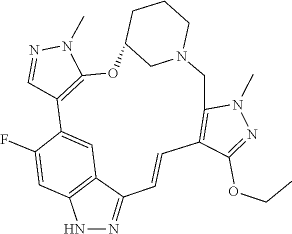 [SMILES: CCOc1nn(C)c2c1/C=C/c1n[nH]c3cc(F)c(cc13)-c1cnn(C)c1O[C@@H]1CCCN(C2)C1] | 2-[(10S,17E)-16-ethoxy-12-ethyl-8,10- dimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 179 | 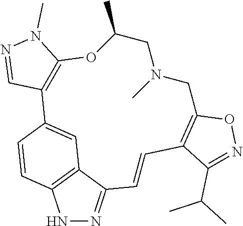 [SMILES: CC(C)c1noc2c1/C=C/c1n[nH]c3ccc(cc13)-c1cnn(C)c1O[C@@H](C)CN(C)C2] | (10S,17E)-8,10,12-trimethyl-16-(propan- 2-yl)-2,8,10,11,12,13-hexahydro-3,5- etheno[1,2]oxazolo[5,4-f]dipyrazolo[3,4- j:4′,3′-n][1,4]oxazacyclopentadecine |
| 180 | 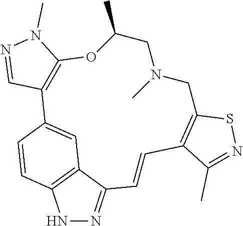 [SMILES: Cc1nsc2c1/C=C/c1n[nH]c3ccc(cc13)-c1cnn(C)c1O[C@@H](C)CN(C)C2] | (10S,17E)-8,10,12-trimethyl-16-(propan- 2-yl)-2,8,10,11,12,13-hexahydro-3,5- etheno[1,2]oxazolo[5,4-f]dipyrazolo[3,4- j:4′,3′-n][1,4]oxazacyclopentadecine |
| 181 | 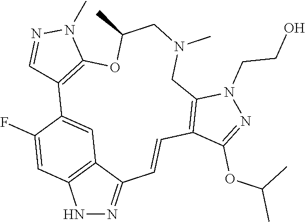 [SMILES: CC(C)Oc1nn(CCO)c2c1/C=C/c1n[nH]c3cc(F)c(cc13)-c1cnn(C)c1O[C@@H](C)CN(C)C2] | 2-{(10S,17E)-19-fluoro-8,10,12- trimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 182 | 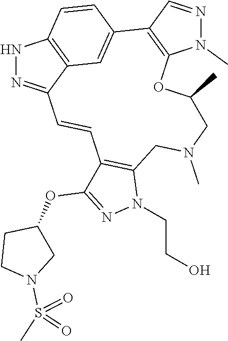 [SMILES: C[C@H]1CN(C)Cc2c(c(O[C@H]3CCN(S(C)(=O)=O)C3)nn2CCO)/C=C/c2n[nH]c3ccc(cc23)-c2cnn(C)c2O1] | 2-[(10S,17E)-16-{[(3S)-1- (methanesulfonyl)pyrrolidin-3-yl]oxy}- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 183 | 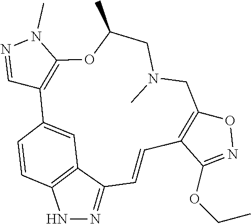 [SMILES: CCOc1noc2c1/C=C/c1n[nH]c3ccc(cc13)-c1cnn(C)c1O[C@@H](C)CN(C)C2] | (10S,17E)-16-ethoxy-8,10,12-trimethyl- 2,8,10,11,12,13-hexahydro-3,5- etheno[1,2]oxazolo[5,4-f]dipyrazolo[3,4- j:4′,3′-n][1,4]oxazacyclopentadecine |
| 184 | 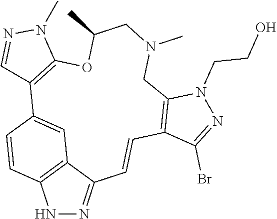 [SMILES: C[C@H]1CN(C)Cc2c(c(Br)nn2CCO)/C=C/c2n[nH]c3ccc(cc23)-c2cnn(C)c2O1] | 2-[(10S,17E)-16-bromo-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 185 | 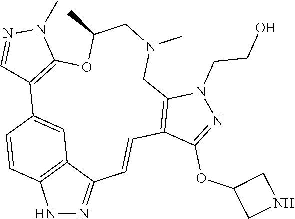 [SMILES: C[C@H]1CN(C)Cc2c(c(OC3CNC3)nn2CCO)/C=C/c2n[nH]c3ccc(cc23)-c2cnn(C)c2O1] | 2-{(10S,17E)-16-[(azetidin-3-yl)oxy]- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl}ethan-1-ol |
| 186 | 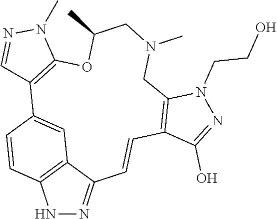 [SMILES: C[C@H]1CN(C)Cc2c(c(O)nn2CCO)/C=C/c2n[nH]c3ccc(cc23)-c2cnn(C)c2O1] | (10S,17E)-14-(2-hydroxyethyl)-8,10,12- trimethyl-2,10,11,12,13,14-hexahydro- 8H-3,5-ethenotripyrazolo[3,4-f:3′,4′- j:4″,3″-n][1,4]oxazacyclopentadecin-16- ol |
| 187 | 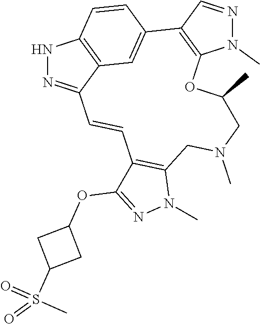 [SMILES: C[C@H]1CN(C)Cc2c(c(OC3CC(S(C)(=O)=O)C3)nn2C)/C=C/c2n[nH]c3ccc(cc23)-c2cnn(C)c2O1] | (10S,17E)-16-{[3- (methanesulfonyl)cyclobutyl]oxy}- 8,10,12,14-tetramethyl-2,10,11,12,13,14- hexahydro-8H-3,5-ethenotripyrazolo[3,4- f:3′,4′-j:4″,3″- n][1,4]oxazacyclopentadecine |
| 188 | 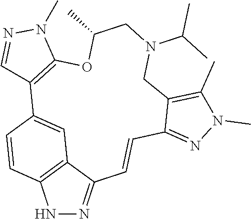 [SMILES: Cc1c2c(nn1C)/C=C/c1n[nH]c3ccc(cc13)-c1cnn(C)c1O[C@H](C)CN(C(C)C)C2] | (10R,17E)-8,10,14,15-tetramethyl-12- (propan-2-yl)-2,10,11,12,13,15- hexahydro-8H-3,5-ethenotripyrazolo[4,3- f:3′,4′-j:4″,3″- n][1,4]oxazacyclopentadecine |
| 189 | 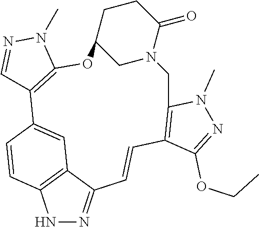 [SMILES: CCOc1nn(C)c2c1/C=C/c1n[nH]c3ccc(cc13)-c1cnn(C)c1O[C@H]1CCC(=O)N(C2)C1] | (10S,19E)-18-ethoxy-8,16-dimethyl- 2,8,11,12,15,16-hexahydro-3,5-etheno- 10,14-methanotripyrazolo[3,4-h:3′,4′- l:4″,3″-p][1,6]oxazacycloheptadecin- 13(10H)-one |
| 190 | 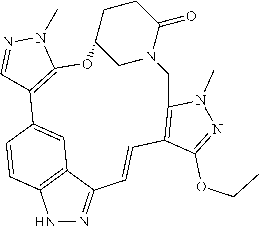 [SMILES: CCOc1nn(C)c2c1/C=C/c1n[nH]c3ccc(cc13)-c1cnn(C)c1O[C@@H]1CCC(=O)N(C2)C1] | (10R,19E)-18-ethoxy-8,16-dimethyl- 2,8,11,12,15,16-hexahydro-3,5-etheno- 10,14-methanotripyrazolo[3,4-h:3′,4′- l:4″,3″-p][1,6]oxazacycloheptadecin- 13(10H)-one |
| 191 | 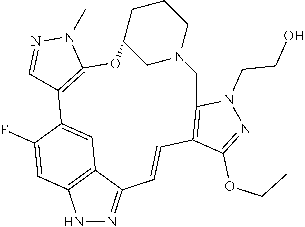 [SMILES: CCOc1nn(CCO)c2c1/C=C/c1n[nH]c3cc(F)c(cc13)-c1cnn(C)c1O[C@@H]1CCCN(C2)C1] | 2-[(10R,19E)-18-ethoxy-22-fluoro-8- methyl-2,10,11,12,13,15-hexahydro-3,5- etheno-10,14-methanotripyrazolo[3,4- h:3′,4′-l:4″,3″- p][1,6]oxazacycloheptadecin-16(8H)- yl]ethan-1-ol |
| 192 | 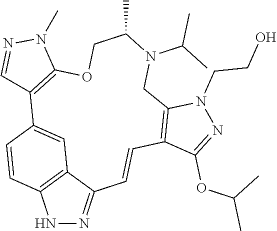 [SMILES: CC(C)Oc1nn(CCO)c2c1/C=C/c1n[nH]c3ccc(cc13)-c1cnn(C)c1OC[C@H](C)N(C(C)C)C2] | 2-{(11S,17E)-8,11-dimethyl-12-(propan- 2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 193 | 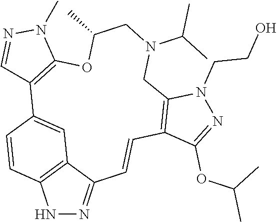 [SMILES: CC(C)Oc1nn(CCO)c2c1/C=C/c1n[nH]c3ccc(cc13)-c1cnn(C)c1O[C@H](C)CN(C(C)C)C2] | 2-{(10R,17E)-8,10-dimethyl-12-(propan- 2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 194 | 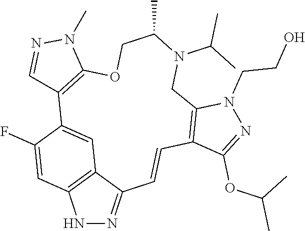 [SMILES: CC(C)Oc1nn(CCO)c2c1/C=C/c1n[nH]c3cc(F)c(cc13)-c1cnn(C)c1OC[C@H](C)N(C(C)C)C2] | 2-{(11S,17E)-19-fluoro-8,11-dimethyl- 12-(propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 195 | 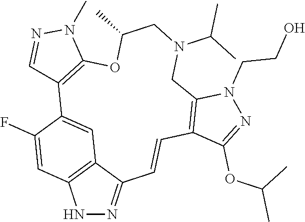 [SMILES: CC(C)Oc1nn(CCO)c2c1/C=C/c1n[nH]c3cc(F)c(cc13)-c1cnn(C)c1O[C@H](C)CN(C(C)C)C2] | 2-{(10R,17E)-19-fluoro-8,10-dimethyl- 12-(propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 196 | 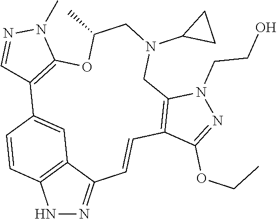 [SMILES: CCOc1nn(CCO)c2c1/C=C/c1n[nH]c3ccc(cc13)-c1cnn(C)c1O[C@H](C)CN(C1CC1)C2] | 2-[(10R,17E)-12-cyclopropyl-16-ethoxy- 8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 197 | 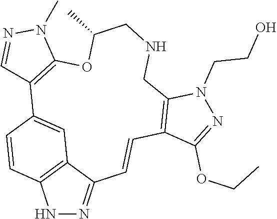 [SMILES: CCOc1nn(CCO)c2c1/C=C/c1n[nH]c3ccc(cc13)-c1cnn(C)c1O[C@H](C)CNC2] | 2-[(10R,17E)-16-ethoxy-8,10-dimethyl- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 198 | 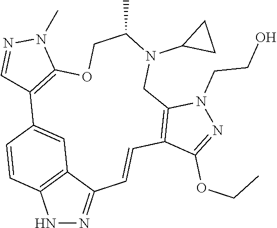 [SMILES: CCOc1nn(CCO)c2c1/C=C/c1n[nH]c3ccc(cc13)-c1cnn(C)c1OC[C@H](C)N(C1CC1)C2] | 2-[(11S,17E)-12-cyclopropyl-16-ethoxy- 8,11-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 199 | 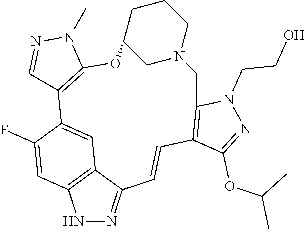 [SMILES: CC(C)Oc1nn(CCO)c2c1/C=C/c1n[nH]c3cc(F)c(cc13)-c1cnn(C)c1O[C@@H]1CCCN(C2)C1] | 2-[(10R,19E)-22-fluoro-8-methyl-18- [(propan-2-yl)oxy]-2,10,11,12,13,15- hexahydro-3,5-etheno-10,14- methanotripyrazolo[3,4-h:3′,4′-l:4″,3″- p][1,6]oxazacycloheptadecin-16(8H)- yl]ethan-1-ol |
| 200 | 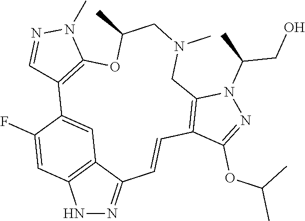 [SMILES: CC(C)Oc1nn([C@@H](C)CO)c2c1/C=C/c1n[nH]c3cc(F)c(cc13)-c1cnn(C)c1O[C@@H](C)CN(C)C2] | (2S)-2-{(10S,17E)-19-fluoro-8,10,12- trimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-1-ol |
| 201 | 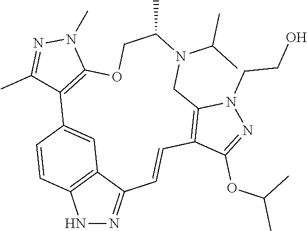 [SMILES: Cc1nn(C)c2c1-c1ccc3[nH]nc(c3c1)/C=C/c1c(OC(C)C)nn(CCO)c1CN(C(C)C)[C@@H](C)CO2] | 2-{(11S,17E)-6,8,11-trimethyl-12- (propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 202 | 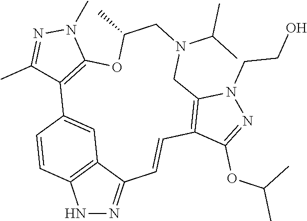 [SMILES: Cc1nn(C)c2c1-c1ccc3[nH]nc(c3c1)/C=C/c1c(OC(C)C)nn(CCO)c1CN(C(C)C)C[C@@H](C)O2] | 2-{(10R,17E)-6,8,10-trimethyl-12- (propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 203 | 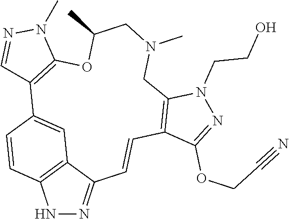 [SMILES: C[C@H]1CN(C)Cc2c(c(OCC#N)nn2CCO)/C=C/c2n[nH]c3ccc(cc23)-c2cnn(C)c2O1] | {[(10S,17E)-14-(2-hydroxyethyl)- 8,10,12-trimethyl-2,10,11,12,13,14- hexahydro-8H-3,5-ethenotripyrazolo[3,4- f:3′,4′-j:4″,3″- n][1,4]oxazacyclopentadecin-16- yl]oxy}acetonitrile |
| 204 | 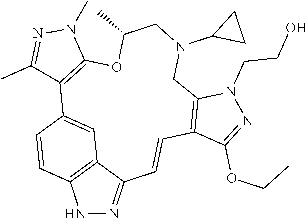 [SMILES: CCOc1nn(CCO)c2c1/C=C/c1n[nH]c3ccc(cc13)-c1c(C)nn(C)c1O[C@H](C)CN(C1CC1)C2] | 2-[(10R,17E)-12-cyclopropyl-16-ethoxy- 6,8,10-trimethyl-2,8,10,11,12,13- hexahydro-14H-3,5- ethenotripyrazolo\|3,4-f:3′,4′-j:4″,3″- n][1,4]oxazacyclopentadecin-14- yl]ethan-1-ol |
| 205 | 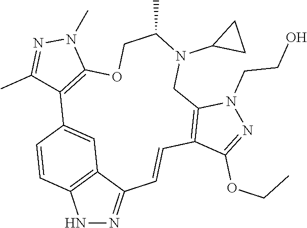 [SMILES: CCOc1nn(CCO)c2c1/C=C/c1n[nH]c3ccc(cc13)-c1c(C)nn(C)c1OC[C@H](C)N(C1CC1)C2] | 2-[(11S,17E)-12-cyclopropyl-16-ethoxy- 6,8,11-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 206 | 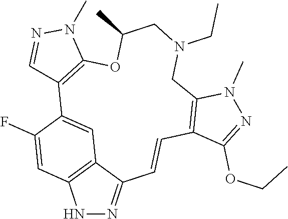 [SMILES: CCOc1nn(C)c2c1/C=C/c1n[nH]c3cc(F)c(cc13)-c1cnn(C)c1O[C@@H](C)CN(CC)C2] | (10S,17E)-16-ethoxy-12-ethyl-19-fluoro- 8,10,14-trimethyl-2,10,11,12,13,14- hexahydro-8H-3,5-ethenotripyrazolo[3,4- f:3′,4′-j:4″,3″- n][1,4]oxazacyclopentadecine |
| 207 | 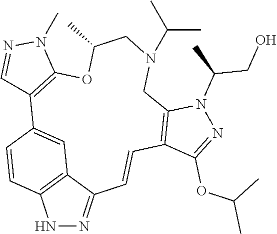 [SMILES: CC(C)Oc1nn([C@@H](C)CO)c2c1/C=C/c1n[nH]c3ccc(cc13)-c1cnn(C)c1O[C@H](C)CN(C(C)C)C2] | (2S)-2-{(10R,17E)-8,10-dimethyl-12- (propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-1-ol |
| 208 | 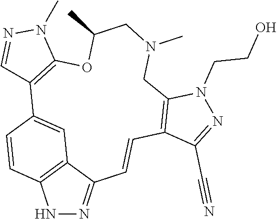 [SMILES: C[C@H]1CN(C)Cc2c(c(C#N)nn2CCO)/C=C/c2n[nH]c3ccc(cc23)-c2cnn(C)c2O1] | (10S,17E)-14-(2-hydroxyethyl)-8,10,12- trimethyl-2,10,11,12,13,14-hexahydro- 8H-3,5-ethenotripyrazolo[3,4-f:3′,4′- j:4″,3″-n][1,4]oxazacyclopentadecine-16- carbonitrile |
| 209 | 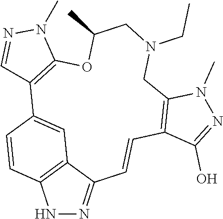 [SMILES: CCN1Cc2c(c(O)nn2C)/C=C/c2n[nH]c3ccc(cc23)-c2cnn(C)c2O[C@@H](C)C1] | (10S,17E)-12-ethyl-8,10,14-trimethyl- 2,10,11,12,13,14-hexahydro-8H-3,5- ethenotripyrazolo[3,4-f:3′,4′-j:4″,3″- n][1,4]oxazacyclopentadecin-16-ol |
| 210 | 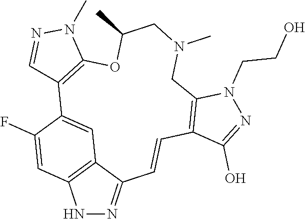 [SMILES: C[C@H]1CN(C)Cc2c(c(O)nn2CCO)/C=C/c2n[nH]c3cc(F)c(cc23)-c2cnn(C)c2O1] | (10S,17E)-19-fluoro-14-(2- hydroxyethyl)-8,10,12-trimethyl- 2,10,11,12,13,14-hexahydro-8H-3,5- ethenotripyrazolo[3,4-f:3′,4′-j:4″,3″- n][1,4]oxazacyclopentadecin-16-ol |
| 211 | 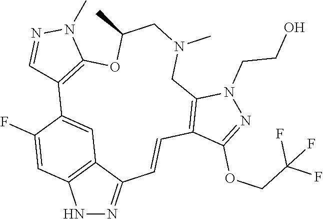 [SMILES: C[C@H]1CN(C)Cc2c(c(OCC(F)(F)F)nn2CCO)/C=C/c2n[nH]c3cc(F)c(cc23)-c2cnn(C)c2O1] | 2-[(10S,17E)-19-fluoro-8,10,12- trimethyl-16-(2,2,2-trifluoroethoxy)- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 212 | 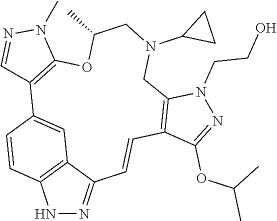 [SMILES: CC(C)Oc1nn(CCO)c2c1/C=C/c1n[nH]c3ccc(cc13)-c1cnn(C)c1O[C@H](C)CN(C1CC1)C2] | 2-{(10R,17E)-12-cyclopropyl-8,10- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 213 | 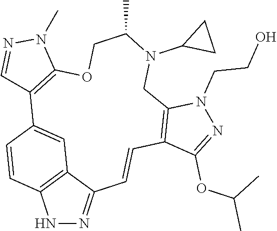 [SMILES: CC(C)Oc1nn(CCO)c2c1/C=C/c1n[nH]c3ccc(cc13)-c1cnn(C)c1OC[C@H](C)N(C1CC1)C2] | 2-{(11S,17E)-12-cyclopropyl-8,11- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 214 | 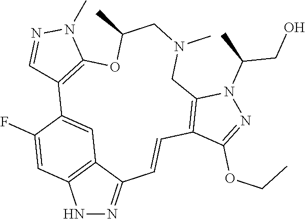 [SMILES: CCOc1nn([C@@H](C)CO)c2c1/C=C/c1n[nH]c3cc(F)c(cc13)-c1cnn(C)c1O[C@@H](C)CN(C)C2] | (2S)-2-[(10S,17E)-16-ethoxy-19-fluoro- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]propan-1-ol |
| 215 | 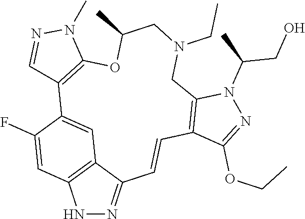 [SMILES: CCOc1nn([C@@H](C)CO)c2c1/C=C/c1n[nH]c3cc(F)c(cc13)-c1cnn(C)c1O[C@@H](C)CN(CC)C2] | (2S)-2-[(10S,17E)-16-ethoxy-12-ethyl- 19-fluoro-8,10-dimethyl-2,8,10,11,12,13- hexahydro-14H-3,5- ethenotripyrazolo\|3,4-f:3′,4′-j:4″,3″- n][1,4]oxazacyclopentadecin-14- yl]propan-1-ol |
| 216 | 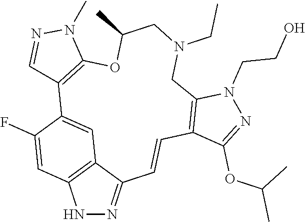 [SMILES: CCN1Cc2c(c(OC(C)C)nn2CCO)/C=C/c2n[nH]c3cc(F)c(cc23)-c2cnn(C)c2O[C@@H](C)C1] | 2-{(10S,17E)-12-ethyl-19-fluoro-8,10- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 217 | 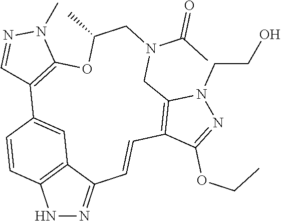 [SMILES: CCOc1nn(CCO)c2c1/C=C/c1n[nH]c3ccc(cc13)-c1cnn(C)c1O[C@H](C)CN(C(C)=O)C2] | 1-[(10R,17E)-16-ethoxy-14-(2- hydroxyethyl)-8,10-dimethyl- 2,8,10,11,13,14-hexahydro-12H-3,5- ethenotripyrazolo[3,4-f:3′,4′-j:4″,3″- n][1,4]oxazacyclopentadecin-12- yl]ethan-1-one |
| 218 | 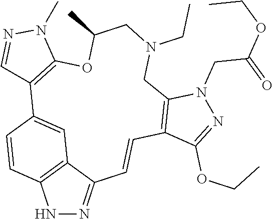 [SMILES: CCOC(=O)Cn1nc(OCC)c2c1CN(CC)C[C@H](C)Oc1c(cnn1C)-c1ccc3[nH]nc(c3c1)/C=C/2] | ethyl[(10S,17E)-16-ethoxy-12-ethyl- 8,10-dimethyl-2,8,10,11,12,13- hexahydro-14H-3,5- ethenotripyrazolo[3,4-f:3′,4′-j:4″,3″- n][1,4]oxazacyclopentadecin-14- yl]acetate |
| 219 | 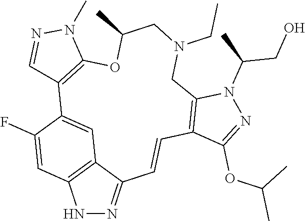 [SMILES: CCN1Cc2c(c(OC(C)C)nn2[C@@H](C)CO)/C=C/c2n[nH]c3cc(F)c(cc23)-c2cnn(C)c2O[C@@H](C)C1] | (2S)-2-{(10S,17E)-12-ethyl-19-fluoro- 8,10-dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-1-ol |
| 220 | 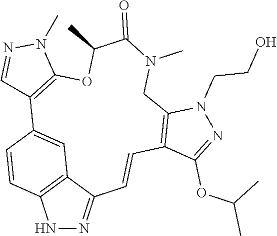 [SMILES: CC(C)Oc1nn(CCO)c2c1/C=C/c1n[nH]c3ccc(cc13)-c1cnn(C)c1O[C@@H](C)C(=O)N(C)C2] | (10S,17E)-14-(2-hydroxyethyl)-8,10,12- trimethyl-16-[(propan-2-yl)oxy]- 2,12,13,14-tetrahydro-8H-3,5- ethenotripyrazolo[3,4-f:3′,4′-j:4″,3″- n][1,4]oxazacyclopentadecin-11(10H)- one |
| 221 | 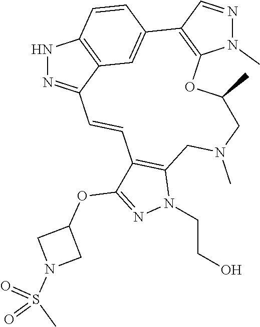 [SMILES: C[C@H]1CN(C)Cc2c(c(OC3CN(S(C)(=O)=O)C3)nn2CCO)/C=C/c2n[nH]c3ccc(cc23)-c2cnn(C)c2O1] | 2-[(10S,17E)-16-{[1- (methanesulfonyl)azetidin-3-ylJoxy}- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 222 | 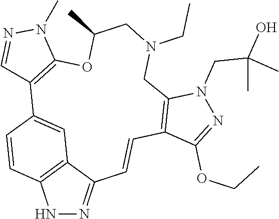 [SMILES: CCOc1nn(CC(C)(C)O)c2c1/C=C/c1n[nH]c3ccc(cc13)-c1cnn(C)c1O[C@@H](C)CN(CC)C2] | 1-[(10S,17E)-16-ethoxy-12-ethyl-8,10- dimethyl-2,8,10,11,12,13-hexahydro- 14H-3,5-ethenotripyrazolo[3,4-f:3′,4′- j:4″,3″-n][1,4]oxazacyclopentadecin-14- yl]-2-methylpropan-2-ol |
| 223 | 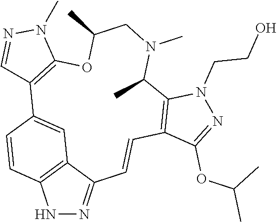 [SMILES: CC(C)Oc1nn(CCO)c2c1/C=C/c1n[nH]c3ccc(cc13)-c1cnn(C)c1O[C@@H](C)CN(C)[C@@H]2C] | 2-{(10S,13R,17E)-8,10,12,13- tetramethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 224 | 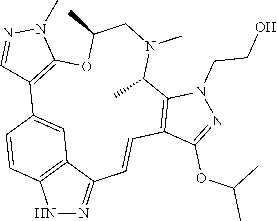 [SMILES: CC(C)Oc1nn(CCO)c2c1/C=C/c1n[nH]c3ccc(cc13)-c1cnn(C)c1O[C@@H](C)CN(C)[C@H]2C] | 2-{(10S,13S,17E)-8,10,12,13- tetramethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 225 | 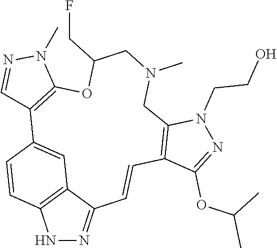 [SMILES: CC(C)Oc1nn(CCO)c2c1/C=C/c1n[nH]c3ccc(cc13)-c1cnn(C)c1OC(CF)CN(C)C2] | 2-{(17E)-10-(fluoromethyl)-8,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 226 | 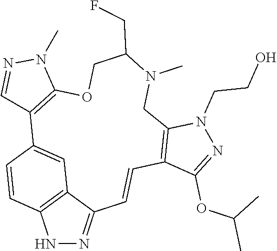 [SMILES: CC(C)Oc1nn(CCO)c2c1/C=C/c1n[nH]c3ccc(cc13)-c1cnn(C)c1OCC(CF)N(C)C2] | 2-{(17E)-11-(fluoromethyl)-8,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 227 | 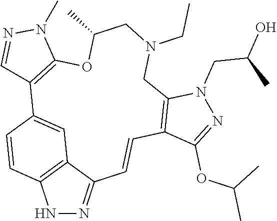 [SMILES: CCN1Cc2c(c(OC(C)C)nn2C[C@H](C)O)/C=C/c2n[nH]c3ccc(cc23)-c2cnn(C)c2O[C@H](C)C1] | (2S)-1-{(10R,17E)-12-ethyl-8,10- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-2-ol |
| 228 | 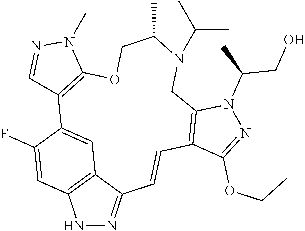 [SMILES: CCOc1nn([C@@H](C)CO)c2c1/C=C/c1n[nH]c3cc(F)c(cc13)-c1cnn(C)c1OC[C@H](C)N(C(C)C)C2] | (2S)-2-[(11S,17E)-16-ethoxy-19-fluoro- 8,11-dimethyl-12-(propan-2-yl)- 2,8,10,11,12,13-hexahydro-14H-3,5- ethenotripyrazolo[3,4-f:3′,4′-j:4″,3″- n][1,4]oxazacyclopentadecin-14- yl]propan-1-ol |
| 229 | 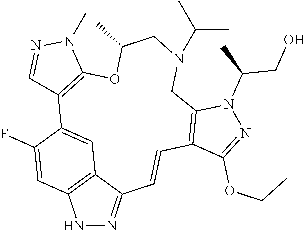 [SMILES: CCOc1nn([C@@H](C)CO)c2c1/C=C/c1n[nH]c3cc(F)c(cc13)-c1cnn(C)c1O[C@H](C)CN(C(C)C)C2] | (2S)-2-[(10R,17E)-16-ethoxy-19-fluoro- 8,10-dimethyl-12-(propan-2-yl)- 2,8,10,11,12,13-hexahydro-14H-3,5- ethenotripyrazolo[3,4-f:3′,4′-j:4″,3″- n][1,4]oxazacyclopentadecin-14- yl]propan-1-ol |
| 230 | 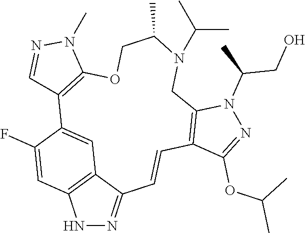 [SMILES: CC(C)Oc1nn([C@@H](C)CO)c2c1/C=C/c1n[nH]c3cc(F)c(cc13)-c1cnn(C)c1OC[C@H](C)N(C(C)C)C2] | (2S)-2-{(11S,17E)-19-fluoro-8,11- dimethyl-12-(propan-2-yl)-16-[(propan- 2-yl)oxy]-2,8,10,11,12,13-hexahydro- 14H-3,5-ethenotripyrazolo[3,4-f:3′,4′- j:4″,3″-n][1,4]oxazacyclopentadecin-14- yl}propan-1-ol |
| 231 | 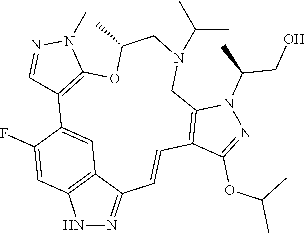 [SMILES: CC(C)Oc1nn([C@@H](C)CO)c2c1/C=C/c1n[nH]c3cc(F)c(cc13)-c1cnn(C)c1O[C@H](C)CN(C(C)C)C2] | (2S)-2-{(10R,17E)-19-fluoro-8,10- dimethyl-12-(propan-2-yl)-16-[(propan- 2-yl)oxy]-2,8,10,11,12,13-hexahydro- 14H-3,5-ethenotripyrazolo[3,4-f:3′,4′- j:4″,3″-n][1,4]oxazacyclopentadecin-14- yl}propan-1-ol |
| 232 | 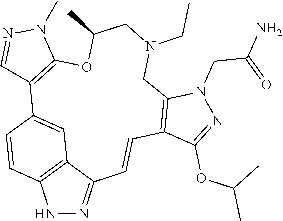 [SMILES: CCN1Cc2c(c(OC(C)C)nn2CC(N)=O)/C=C/c2n[nH]c3ccc(cc23)-c2cnn(C)c2O[C@@H](C)C1] | 2-{(10S,17E)-12-ethyl-8,10-dimethyl-16- [(propan-2-yl)oxy]-2,8,10,11,12,13- hexahydro-14H-3,5- ethenotripyrazolo[3,4-f:3′,4′-j:4″,3″- n][1,4]oxazacyclopentadecin-14- yl}acetamide |
| 233 | 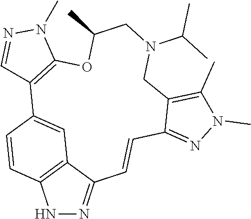 [SMILES: Cc1c2c(nn1C)/C=C/c1n[nH]c3ccc(cc13)-c1cnn(C)c1O[C@@H](C)CN(C(C)C)C2] | (10S,17E)-8,10,14,15-tetramethyl-12- (propan-2-yl)-2,10,11,12,13,15- hexahydro-8H-3,5-ethenotripyrazolo[4,3- f:3′,4′-j:4″,3″- n][1,4]oxazacyclopentadecine |
| 234 | 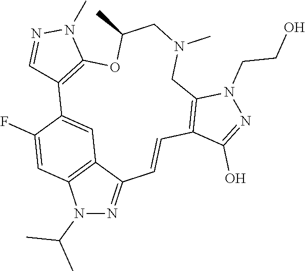 [SMILES: CC(C)n1nc2c3cc(c(F)cc31)-c1cnn(C)c1O[C@@H](C)CN(C)Cc1c(c(O)nn1CCO)/C=C/2] | (10S,17E)-19-fluoro-14-(2- hydroxyethyl)-8,10,12-trimethyl-2- (propan-2-yl)-2,10,11,12,13,14- hexahydro-8H-3,5-ethenotripyrazolo[3,4- f:3′,4′-j:4″,3″- n][1,4]oxazacyclopentadecin-16-ol |
| 235 | 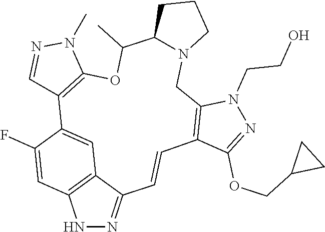 [SMILES: CC1Oc2c(cnn2C)-c2cc3c(n[nH]c3cc2F)/C=C/c2c(OCC3CC3)nn(CCO)c2CN2CCC[C@H]12] | 2-[(8aR,19E)-1-(cyclopropylmethoxy)- 22-fluoro-9,11-dimethyl-7,8,8a,9,11,17- hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 236 | 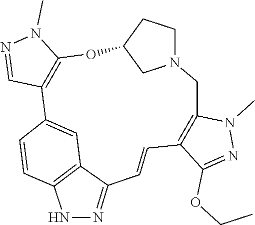 [SMILES: CCOc1nn(C)c2c1/C=C/c1n[nH]c3ccc(cc13)-c1cnn(C)c1O[C@@H]1CCN(C2)C1] | (10R,18E)-17-ethoxy-8,15-dimethyl- 2,8,11,12,14,15-hexahydro-10H-3,5- etheno-10,13-methanotripyrazolo[3,4- g:3′,4′-k:4″,3″- o][1,5]oxazacyclohexadecine |
| 237 | 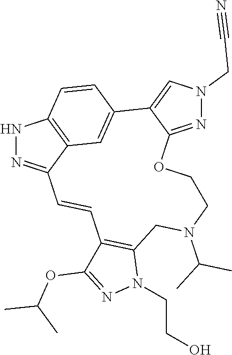 [SMILES: CC(C)Oc1nn(CCO)c2c1/C=C/c1n[nH]c3ccc(cc13)-c1cn(CC#N)nc1OCCN(C(C)C)C2] | {(17E)-14-(2-hydroxyethyl)-12-(propan- 2-yl)-16-[(propan-2-yl)oxy]- 2,10,11,12,13,14-hexahydro-7H-3,5- ethenotripyrazolo[3,4-f:3′,4′-j:4″,3″- n][1,4]oxazacyclopentadecin-7- yl}acetonitrile |
| 238 | 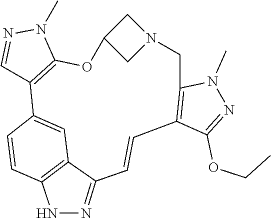 [SMILES: CCOc1nn(C)c2c1/C=C/c1n[nH]c3ccc(cc13)-c1cnn(C)c1OC1CN(C2)C1] | (17E)-16-ethoxy-8,14-dimethyl- 2,8,10,11,13,14-hexahydro-3,5-etheno- 10,12-methanotripyrazolo[3,4-f:3′,4′- j:4″,3″-n][1,4]oxazacyclopentadecine |
| 239 | 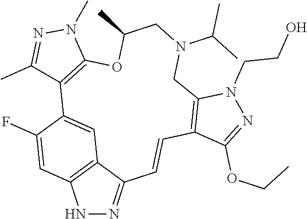 [SMILES: CCOc1nn(CCO)c2c1/C=C/c1n[nH]c3cc(F)c(cc13)-c1c(C)nn(C)c1O[C@@H](C)CN(C(C)C)C2] | 2-[(10S,17E)-16-ethoxy-19-fluoro- 6,8,10-trimethyl-12-(propan-2-yl)- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 240 | 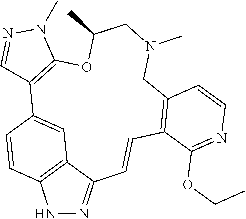 [SMILES: CCOc1nccc2c1/C=C/c1n[nH]c3ccc(cc13)-c1cnn(C)c1O[C@@H](C)CN(C)C2] | (10S,18E)-17-ethoxy-8,10,12-trimethyl- 2,8,10,11,12,13-hexahydro-3,5- ethenodipyrazolo[3,4-j:4′,3′-n]pyrido[4,3- f][1,4]oxazacyclopentadecine |
| 241 | 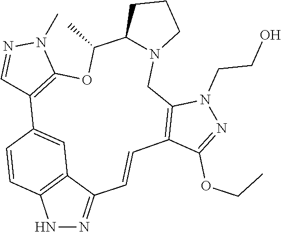 [SMILES: CCOc1nn(CCO)c2c1/C=C/c1n[nH]c3ccc(cc13)-c1cnn(C)c1O[C@H](C)[C@H]1CCCN1C2] | 2-[(8aR,9R,19E)-1-ethoxy-9,11-dimethyl- 7,8,8a,9,11,17-hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 241 | 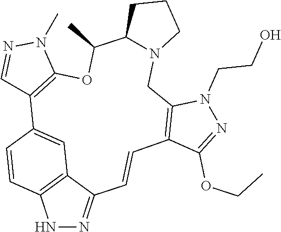 [SMILES: CCOc1nn(CCO)c2c1/C=C/c1n[nH]c3ccc(cc13)-c1cnn(C)c1O[C@@H](C)[C@H]1CCCN1C2] | 2-[(8aR,9S,19E)-1-ethoxy-9,11-dimethyl- 7,8,8a,9,11,17-hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 242 | 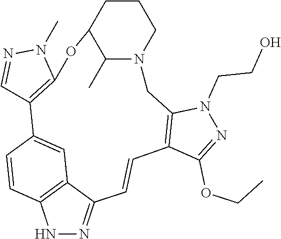 [SMILES: CCOc1nn(CCO)c2c1/C=C/c1n[nH]c3ccc(cc13)-c1cnn(C)c1OC1CCCN(C2)C1C] | 2-[(19E)-18-ethoxy-8,21-dimethyl- 2,10,11,12,13,15-hexahydro-3,5-etheno- 10,14-methanotripyrazolo[3,4-h:3′,4′- l:4″,3″-p][1,6]oxazacycloheptadecin- 16(8H)-yl]ethan-1-ol |
| 243 | 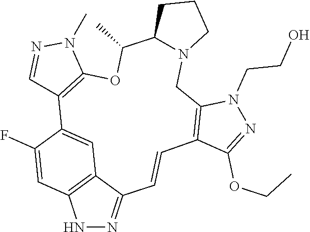 [SMILES: CCOc1nn(CCO)c2c1/C=C/c1n[nH]c3cc(F)c(cc13)-c1cnn(C)c1O[C@H](C)[C@H]1CCCN1C2] | 2-[(8aR,9R,19E)-1-ethoxy-22-fluoro- 9,11-dimethyl-7,8,8a,9,11,17-hexahydro- 6H-14,16-ethenotripyrazolo[3,4-f:3′,4′- j:4″,3″-n]pyrrolo[2,1- c][1,4]oxazacyclopentadecin-3(4H)- yl]ethan-1-ol |
| 244 | 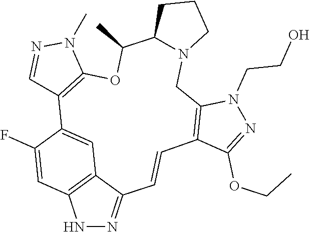 [SMILES: CCOc1nn(CCO)c2c1/C=C/c1n[nH]c3cc(F)c(cc13)-c1cnn(C)c1O[C@@H](C)[C@H]1CCCN1C2] | 2-[(8aR,9S,19E)-1-ethoxy-22-fluoro- 9,11-dimethyl-7,8,8a,9,11,17-hexahydro- 6H-14,16-ethenotripyrazolo[3,4-f:3′,4′- j:4″,3″-n]pyrrolo[2,1- c][1,4]oxazacyclopentadecin-3(4H)- yl]ethan-1-ol |
| 245 | 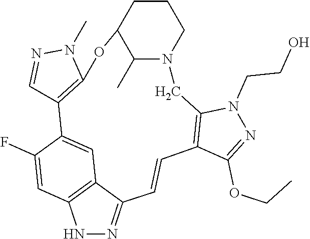 [SMILES: CCOc1nn(CCO)c2c1/C=C/c1n[nH]c3cc(F)c(cc13)-c1cnn(C)c1OC1CCCN(C2)C1C] | 2-[(19E)-18-ethoxy-22-fluoro-8,21- dimethyl-2,10,11,12,13,15-hexahydro- 3,5-etheno-10,14- methanotripyrazolo[3,4-h:3′,4′-l:4″,3″- p][1,6]oxazacycloheptadecin-16(8H)- yl]ethan-1-ol |
| 246 | 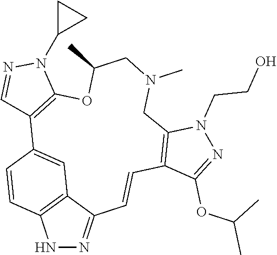 [SMILES: CC(C)Oc1nn(CCO)c2c1/C=C/c1n[nH]c3ccc(cc13)-c1cnn(C3CC3)c1O[C@@H](C)CN(C)C2] | 2-{(10S,17E)-8-cyclopropyl-10,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 247 | 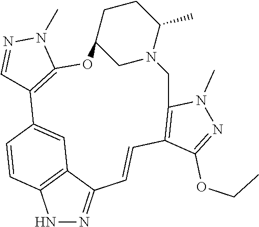 [SMILES: CCOc1nn(C)c2c1/C=C/c1n[nH]c3ccc(cc13)-c1cnn(C)c1O[C@H]1CC[C@H](C)N(C2)C1] | (10S,13S,19E)-18-ethoxy-8,13,16- trimethyl-2,8,10,11,12,13,15,16- octahydro-3,5-etheno-10,14- methanotripyrazolo[3,4-h:3′,4′-l:4″,3″- p][1,6]oxazacycloheptadecine |
| 248 | 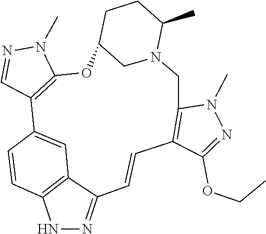 | (10R,13R,19E)-18-ethoxy-8,13,16- trimethyl-2,8,10,11,12,13,15,16- octahydro-3,5-etheno-10,14- methanotripyrazolo[3,4-h:3′,4′-l:4″,3″- p][1,6]oxazacycloheptadecine |
| 249 | 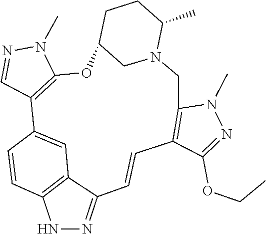 | (10R,13S,19E)-18-ethoxy-8,13,16- trimethyl-2,8,10,11,12,13,15,16- octahydro-3,5-etheno-10,14- methanotripyrazolo[3,4-h:3′,4′-l:4″,3″- p][1,6]oxazacycloheptadecine |
| 250 | 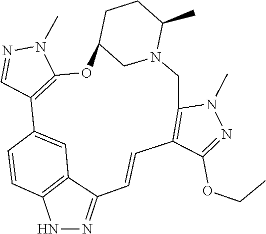 | (10S,13R,19E)-18-ethoxy-8,13,16- trimethyl-2,8,10,11,12,13,15,16- octahydro-3,5-etheno-10,14- methanotripyrazolo[3,4-h:3′,4′-l:4″,3″- p][1,6]oxazacycloheptadecine |
| 251 | 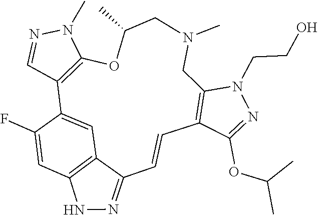 | 2-{(10R,17E)-19-fluoro-8,10,12- trimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 252 | 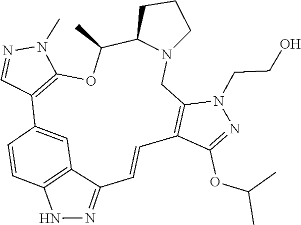 | 2-[(8aR,9S,19E)-9,11-dimethyl-1- [(propan-2-yl)oxy]-7,8,8a,9,11,17- hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 253 | 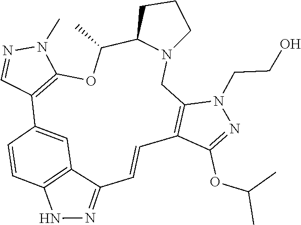 | 2-[(8aR,9R,19E)-9,11-dimethyl-1- [(propan-2-yl)oxy]-7,8,8a,9,11,17- hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 254 | 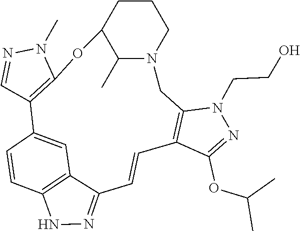 | 2-[(19E)-8,21-dimethyl-18-[(propan-2- yl)oxy]-2,10,11,12,13,15-hexahydro-3,5- etheno-10,14-methanotripyrazolo[3,4- h:3′,4′-l:4″,3″- p][1,6]oxazacycloheptadecin-16(8H)- yl]ethan-1-ol |
| 255 | 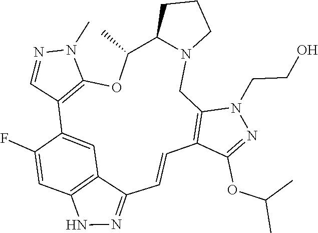 [SMILES: CC(C)Oc1nn(CCO)c2c1/C=C/c1n[nH]c3cc(F)c(cc13)-c1cnn(C)c1O[C@H](C)[C@H]1CCCN1C2] | 2-[(8aR,9R,19E)-22-fluoro-9,11- dimethyl-1-[(propan-2-yl)oxy]- 7,8,8a,9,11,17-hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 256 | 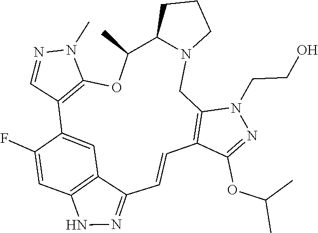 [SMILES: CC(C)Oc1nn(CCO)c2c1/C=C/c1n[nH]c3cc(F)c(cc13)-c1cnn(C)c1O[C@@H](C)[C@H]1CCCN1C2] | 2-[(8aR,9S,19E)-22-fluoro-9,11- dimethyl-1-[(propan-2-yl)oxy]- 7,8,8a,9,11,17-hexahydro-6H-14,16- ethenotripyrazolo[3,4-f:3′,4′-j:4″,3″- n]pyrrolo[2,1- c][1,4]oxazacyclopentadecin-3(4H)- yl]ethan-1-ol |
| 257 | 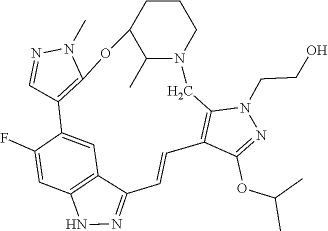 | 2-[(19E)-22-fluoro-8,21-dimethyl-18- [(propan-2-yl)oxy]-2,10,11,12,13,15- hexahydro-3,5-etheno-10,14- methanotripyrazolo[3,4-h:3′,4′-l:4″,3″- p][1,6]oxazacycloheptadecin-16(8H)- yl]ethan-1-ol |
| 258 | 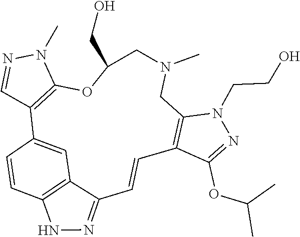 | 2-{(10R,17E)-10-(hydroxymethyl)-8,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 259 | 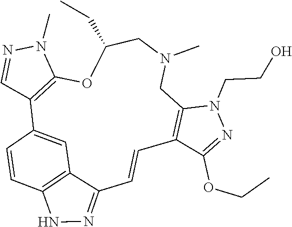 [SMILES: CCOc1nn(CCO)c2c1/C=C/c1n[nH]c3ccc(cc13)-c1cnn(C)c1O[C@H](CC)CN(C)C2] | 2-[(11S,17E)-16-ethoxy-11-ethyl-8,12- dimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 260 | 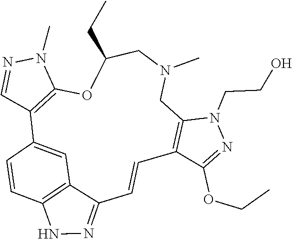 [SMILES: CCOc1nn(CCO)c2c1/C=C/c1n[nH]c3ccc(cc13)-c1cnn(C)c1O[C@@H](CC)CN(C)C2] | 2-[(10S,17E)-16-ethoxy-10-ethyl-8,12- dimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 261 | 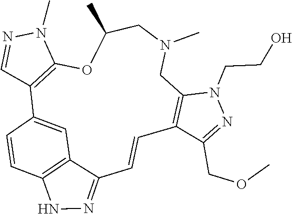 [SMILES: COCc1nn(CCO)c2c1/C=C/c1n[nH]c3ccc(cc13)-c1cnn(C)c1O[C@@H](C)CN(C)C2] | 2-[(10S,17E)-16-(methoxymethyl)- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 262 | 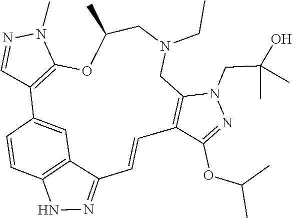 [SMILES: CCN1Cc2c(c(OC(C)C)nn2CC(C)(C)O)/C=C/c2n[nH]c3ccc(cc23)-c2cnn(C)c2O[C@@H](C)C1] | 1-{(10S,17E)-12-ethyl-8,10-dimethyl-16- [(propan-2-yl)oxy]-2,8,10,11,12,13- hexahydro-14H-3,5- ethenotripyrazolo[3,4-f:3′,4′-j:4″,3″- n][1,4]oxazacyclopentadecin-14-yl}-2- methylpropan-2-ol |
| 263 | 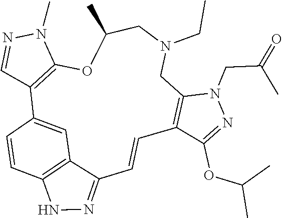 [SMILES: CCN1Cc2c(c(OC(C)C)nn2CC(C)=O)/C=C/c2n[nH]c3ccc(cc23)-c2cnn(C)c2O[C@@H](C)C1] | 1-{(10S,17E)-12-ethyl-8,10-dimethyl-16- [(propan-2-yl)oxy]-2,8,10,11,12,13- hexahydro-14H-3,5- ethenotripyrazolo[3,4-f:3′,4′-j:4″,3″- n][1,4]oxazacyclopentadecin-14- yl}propan-2-one |
| 264 | 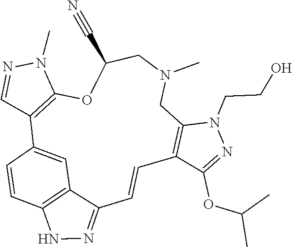 [SMILES: CC(C)Oc1nn(CCO)c2c1/C=C/c1n[nH]c3ccc(cc13)-c1cnn(C)c1O[C@@H](C#N)CN(C)C2] | (10R,17E)-14-(2-hydroxyethyl)-8,12- dimethyl-16-[(propan-2-yl)oxy]- 2,10,11,12,13,14-hexahydro-8H-3,5- ethenotripyrazolo[3,4-f:3′,4′-j:4″,3″- n][1,4]oxazacyclopentadecine-10- carbonitrile |
| 265 | 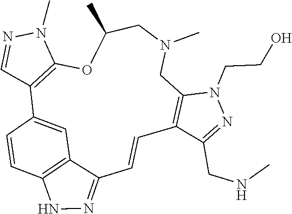 [SMILES: CNCc1nn(CCO)c2c1/C=C/c1n[nH]c3ccc(cc13)-c1cnn(C)c1O[C@@H](C)CN(C)C2] | 2-{(10S,17E)-8,10,12-trimethyl-16- [(methylamino)methyl]-2,8,10,11,12,13- hexahydro-14H-3,5- ethenotripyrazolo[3,4-f:3′,4′-j:4″,3″- n][1,4]oxazacyclopentadecin-14- yl}ethan-1-ol |
| 266 | 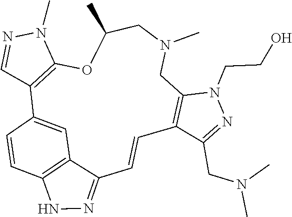 [SMILES: C[C@H]1CN(C)Cc2c(c(CN(C)C)nn2CCO)/C=C/c2n[nH]c3ccc(cc23)-c2cnn(C)c2O1] | 2-{(10S,17E)-16- [(dimethylamino)methyl]-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl}ethan-1-ol |
| 267 | 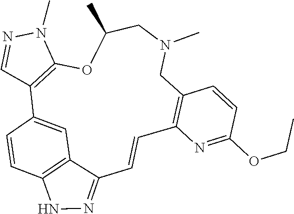 [SMILES: CCOc1ccc2c(n1)/C=C/c1n[nH]c3ccc(cc13)-c1cnn(C)c1O[C@@H](C)CN(C)C2] | (10S,18E)-16-ethoxy-8,10,12-trimethyl- 2,8,10,11,12,13-hexahydro-3,5- ethenodipyrazolo[3,4-j:4′,3′-n]pyrido[3,2- f][1,4]oxazacyclopentadecine |
| 268 | 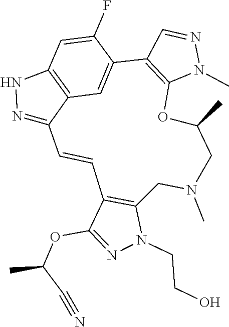 [SMILES: C[C@H](C#N)Oc1nn(CCO)c2c1/C=C/c1n[nH]c3cc(F)c(cc13)-c1cnn(C)c1O[C@@H](C)CN(C)C2] | (2R)-2-{[(10S,17E)-19-fluoro-14-(2- hydroxyethyl)-8,10,12-trimethyl- 2,10,11,12,13,14-hexahydro-8H-3,5- ethenotripyrazolo[3,4-f:3′,4′-j:4″,3″- n][1,4]oxazacyclopentadecin-16- yl]oxy}propanenitrile |
| 269 | 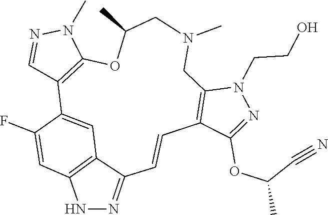 [SMILES: C[C@@H](C#N)Oc1nn(CCO)c2c1/C=C/c1n[nH]c3cc(F)c(cc13)-c1cnn(C)c1O[C@@H](C)CN(C)C2] | (2S)-2-{[(10S,17E)-19-fluoro-14-(2- hydroxyethyl)-8,10,12-trimethyl- 2,10,11,12,13,14-hexahydro-8H-3,5- ethenotripyrazolo[3,4-f:3′,4′-j:4″,3″- n][1,4]oxazacyclopentadecin-16- yl]oxy}propanenitrile |
| 270 | 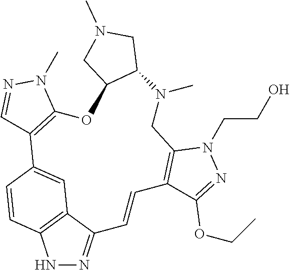 [SMILES: CCOc1nn(CCO)c2c1/C=C/c1n[nH]c3ccc(cc13)-c1cnn(C)c1O[C@H]1CN(C)C[C@@H]1N(C)C2] | 2-[(4aS,7aS,13E)-12-ethoxy-3,6,8- trimethyl-4a,5,6,7,7a,8,9,16-octahydro- 17,19-ethenotripyrazolo[3,4-f:3′,4′- j:4″,3″-n\|pyrrolo\|3,4- b][1,4]oxazacyclopentadecin-10(3H)- yl]ethan-1-ol |
| 271 | 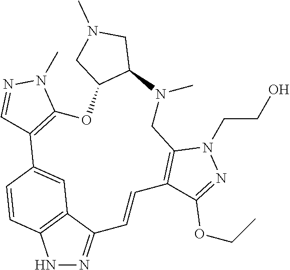 [SMILES: CCOc1nn(CCO)c2c1/C=C/c1n[nH]c3ccc(cc13)-c1cnn(C)c1O[C@@H]1CN(C)C[C@H]1N(C)C2] | 2-[(4aR,7aR,13E)-12-ethoxy-3,6,8- trimethyl-4a,5,6,7,7a,8,9,16-octahydro- 17,19-ethenotripyrazolo[3,4-f:3′,4′- j:4″,3″-n]pyrrolo[3,4- b][1,4]oxazacyclopentadecin-10(3H)- yl]ethan-1-ol |
| 272 | 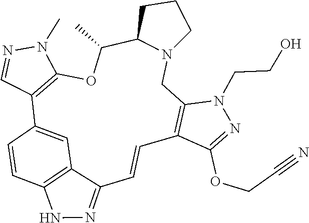 | {[(8aR,19E)-3-(2-hydroxyethyl)-9,11- dimethyl-3,4,7,8,8a,9,11,17-octahydro- 6H-14,16-ethenotripyrazolo[3,4-f:3′,4′- j:4″,3″-n]pyrrolo[2,1- c][1,4]oxazacyclopentadecin-1- ylJoxy}acetonitrile |
| 273 | 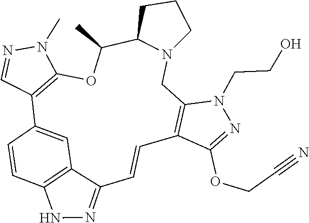 | {[(8aR,9R,19E)-3-(2-hydroxyethyl)-9,11- dimethyl-3,4,7,8,8a,9,11,17-octahydro- 6H-14,16-ethenotripyrazolo[3,4-f:3′,4′- j:4″,3″-n]pyrrolo[2,1- c][1,4]oxazacyclopentadecin-1- yl]oxy}acetonitrile |
| 274 | 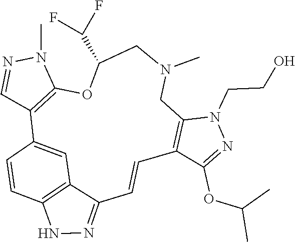 [SMILES: CC(C)Oc1nn(CCO)c2c1/C=C/c1n[nH]c3ccc(cc13)-c1cnn(C)c1O[C@H](C(F)F)CN(C)C2] | 2-{(10S,17E)-10-(difluoromethyl)-8,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 275 | 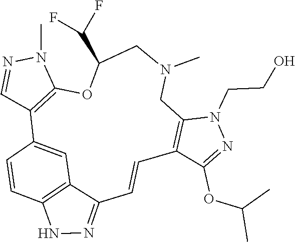 [SMILES: CC(C)Oc1nn(CCO)c2c1/C=C/c1n[nH]c3ccc(cc13)-c1cnn(C)c1O[C@@H](C(F)F)CN(C)C2] | 2-{(10R,17E)-10-(difluoromethyl)-8,12- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 276 | 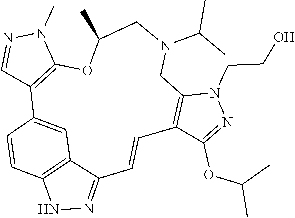 [SMILES: CC(C)Oc1nn(CCO)c2c1/C=C/c1n[nH]c3ccc(cc13)-c1cnn(C)c1O[C@@H](C)CN(C(C)C)C2] | 2-{(10S,17E)-8,10-dimethyl-12-(propan- 2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 277 | 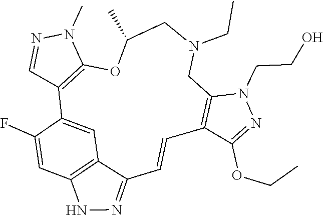 [SMILES: CCOc1nn(CCO)c2c1/C=C/c1n[nH]c3cc(F)c(cc13)-c1cnn(C)c1O[C@H](C)CN(CC)C2] | 2-[(10R,17E)-16-ethoxy-12-ethyl-19- fluoro-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 278 | 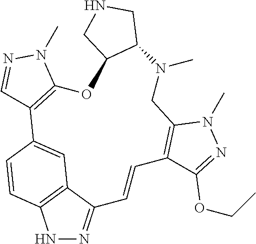 [SMILES: CCOc1nn(C)c2c1/C=C/c1n[nH]c3ccc(cc13)-c1cnn(C)c1O[C@H]1CNC[C@@H]1N(C)C2] | (4aS,7aS,13E)-12-ethoxy-3,8,10- trimethyl-3,4a,5,6,7,7a,8,9,10,16- decahydro-17,19-ethenotripyrazolo[3,4- f:3′,4′-j:4″,3″-n]pyrrolo[3,4- b][1,4]oxazacyclopentadecine |
| 279 | 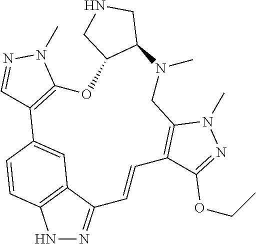 [SMILES: CCOc1nn(C)c2c1/C=C/c1n[nH]c3ccc(cc13)-c1cnn(C)c1O[C@@H]1CNC[C@H]1N(C)C2] | (4aR,7aR,13E)-12-ethoxy-3,8,10- trimethyl-3,4a,5,6,7,7a,8,9,10,16- decahydro-17,19-ethenotripyrazolo[3,4- f:3′,4′-j:4″,3″-n]pyrrolo[3,4- b][1,4]oxazacyclopentadecine |
| 280 | 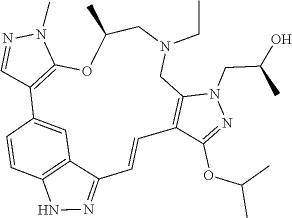 [SMILES: CCN1Cc2c(c(OC(C)C)nn2C[C@H](C)O)/C=C/c2n[nH]c3ccc(cc23)-c2cnn(C)c2O[C@@H](C)C1] | (2S)-1-{(10S,17E)-12-ethyl-8,10- dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-2-ol |
| 281 | 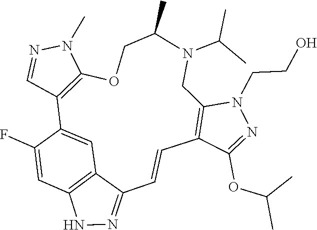 | 2-{(11R,17E)-19-fluoro-8,11-dimethyl- 12-(propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 282 | 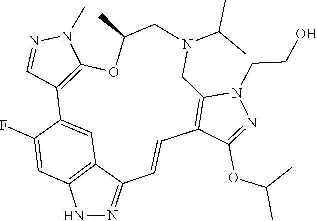 | 2-{(10S,17E)-19-fluoro-8,10-dimethyl- 12-(propan-2-yl)-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}ethan-1-ol |
| 283 | 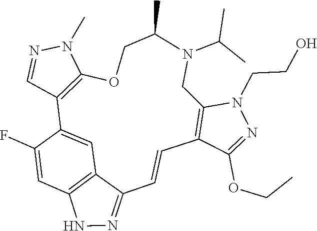 | 2-[(11R,17E)-16-ethoxy-19-fluoro-8,11- dimethyl-12-(propan-2-yl)- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 284 | 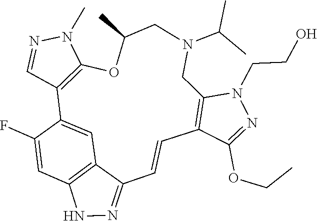 | 2-[(10S,17E)-16-ethoxy-19-fluoro-8,10- dimethyl-12-(propan-2-yl)- 2,8,10,11,12,13-hexahydro-14H-3,5- ethenotripyrazolo[3,4-f:3′,4′-j:4″,3″- n][1,4]oxazacyclopentadecin-14- yl]ethan-1-ol |
| 285 | 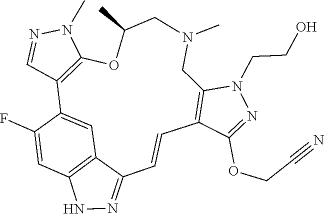 | {[(10S,17E)-19-fluoro-14-(2- hydroxyethyl)-8,10,12-trimethyl- 2,10,11,12,13,14-hexahydro-8H-3,5- ethenotripyrazolo[3,4-f:3′,4′-j:4″,3″- n][1,4]oxazacyclopentadecin-16- yl]oxy}acetonitrile |
| 286 | 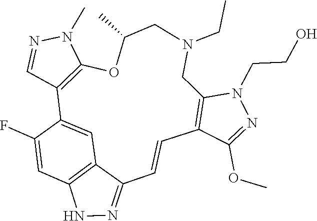 | 2-[(10R,17E)-12-ethyl-19-fluoro-16- methoxy-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 287 | 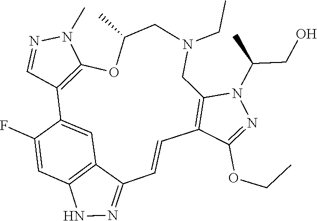 | (2S)-2-[(10R,17E)-16-ethoxy-12-ethyl- 19-fluoro-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]propan-1-ol |
| 288 | 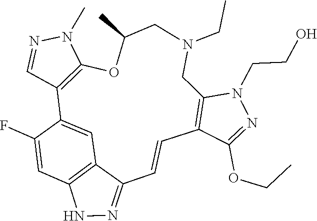 | 2-[(10S,17E)-16-ethoxy-12-ethyl-19- fluoro-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 289 | 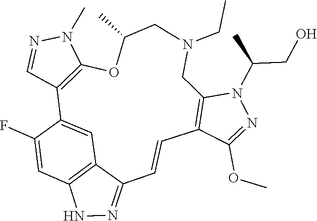 | (2S)-2-[(10R,17E)-12-ethyl-19-fluoro-16- methoxy-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]propan-1-ol |
| 290 | 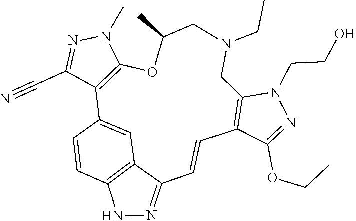 | (10S,17E)-16-ethoxy-12-ethyl-14-(2- hydroxyethyl)-8,10-dimethyl- 2,10,11,12,13,14-hexahydro-8H-3,5- ethenotripyrazolo[3,4-f:3′,4′-j:4″,3″- n][1,4]oxazacyclopentadecine-6- carbonitrile |
| 291 | 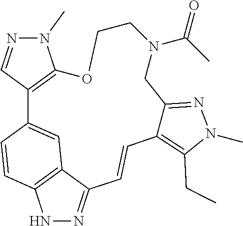 [SMILES: CCc1c2c(nn1C)CN(C(C)=O)CCOc1c(cnn1C)-c1ccc3[nH]nc(c3c1)/C=C/2] | 1-[(17E)-16-ethyl-8,15-dimethyl- 2,8,10,11,13,15-hexahydro-12H-3,5- ethenotripyrazolo[3,4-f:3′,4′-j:4″,3″- n][1,4]oxazacyclopentadecin-12- yl]ethan-1-one |
| 292 | 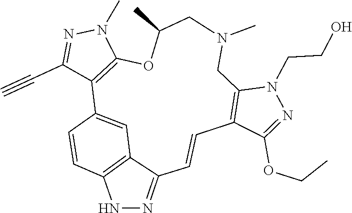 [SMILES: C#Cc1nn(C)c2c1-c1ccc3[nH]nc(c3c1)/C=C/c1c(OCC)nn(CCO)c1CN(C)C[C@H](C)O2] | 2-[(10S,17E)-16-ethoxy-6-ethynyl- 8,10,12-trimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 293 | 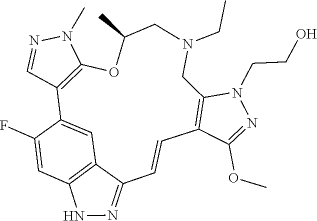 | 2-[(10S,17E)-12-ethyl-19-fluoro-16- methoxy-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 294 | 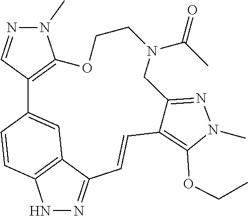 [SMILES: CCOc1c2c(nn1C)CN(C(C)=O)CCOc1c(cnn1C)-c1ccc3[nH]nc(c3c1)/C=C/2] | 1-[(17E)-16-ethoxy-8,15-dimethyl- 2,8,10,11,13,15-hexahydro-12H-3,5- ethenotripyrazolo[3,4-f:3′,4′-j:4″,3″- n][1,4]oxazacyclopentadecin-12- yl]ethan-1-one |
| 295 | 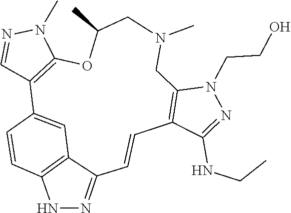 [SMILES: CCNc1nn(CCO)c2c1/C=C/c1n[nH]c3ccc(cc13)-c1cnn(C)c1O[C@@H](C)CN(C)C2] | 2-[(10S,17E)-16-(ethylamino)-8,10,12- trimethyl-2,8,10,11,12,13-hexahydro- 14H-3,5-ethenotripyrazolo[3,4-f:3′,4′- j:4″,3″-n][1,4]oxazacyclopentadecin-14- yl]ethan-1-ol |
| 296 | 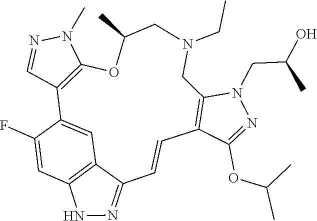 | (2S)-1-{(10S,17E)-12-ethyl-19-fluoro- 8,10-dimethyl-16-[(propan-2-yl)oxy]- 2,8,10,11,12,13-hexahydro-14H-3,5- ethenotripyrazolo[3,4-f:3′,4′-j:4″,3″- n][1,4]oxazacyclopentadecin-14- yl}propan-2-ol |
| 297 | 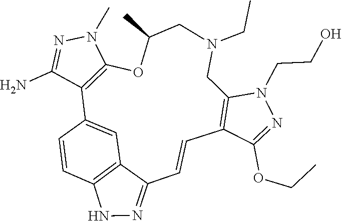 | 2-[(10S,17E)-6-amino-16-ethoxy-12- ethyl-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]ethan-1-ol |
| 298 | 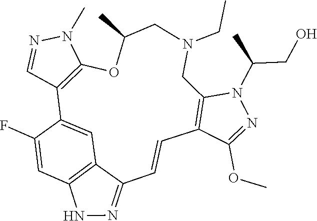 | (2S)-2-[(10S,17E)-12-ethyl-19-fluoro-16- methoxy-8,10-dimethyl-2,8,10,11,12,13- hexahydro-14H-3,5- ethenotripyrazolo[3,4-f:3′,4′-j:4″,3″- n][1,4]oxazacyclopentadecin-14- yl]propan-1-ol |
| 299 | 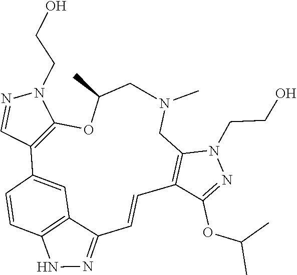 [SMILES: CC(C)Oc1nn(CCO)c2c1/C=C/c1n[nH]c3ccc(cc13)-c1cnn(CCO)c1O[C@@H](C)CN(C)C2] | 2,2′-[(10S,17E)-10,12-dimethyl-16- [(propan-2-yl)oxy]-10,11,12,13- tetrahydro-8H-3,5-ethenotripyrazolo[3,4- f:3′,4′-j:4″,3″- n][1,4]oxazacyclopentadecine-8,14(2H)- diyl]di(ethan-1-ol) |
| 300 | 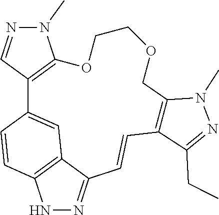 [SMILES: CCc1nn(C)c2c1/C=C/c1n[nH]c3ccc(cc13)-c1cnn(C)c1OCCOC2] | (17E)-16-ethyl-8,14-dimethyl- 2,8,10,11,13,14-hexahydro-3,5- etheno[1,4]dioxacyclopentadecino[10,11- c:15,14-c′:6,7-c″]tripyrazole |
| 301 | 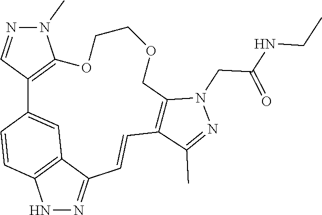 [SMILES: CCNC(=O)Cn1nc(C)c2c1COCCOc1c(cnn1C)-c1ccc3[nH]nc(c3c1)/C=C/2] | 2-[(17E)-8,16-dimethyl-2,10,11,13- tetrahydro-3,5- etheno[1,4]dioxacyclopentadecino[10,11- c:15,14-c′:6,7-c″]tripyrazol-14(8H)-yl]- N-ethylacetamide |
| 302 | 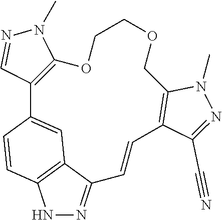 [SMILES: Cn1nc(C#N)c2c1COCCOc1c(cnn1C)-c1ccc3[nH]nc(c3c1)/C=C/2] | (17E)-8,14-dimethyl-2,8,10,11,13,14- hexahydro-3,5- etheno[1,4]dioxacyclopentadecino[10,11- c:15,14-c′:6,7-c″]tripyrazole-16- carbonitrile |
| 303 | 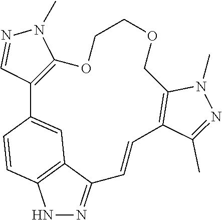 [SMILES: Cc1nn(C)c2c1/C=C/c1n[nH]c3ccc(cc13)-c1cnn(C)c1OCCOC2] | (17E)-8,14,16-trimethyl-2,8,10,11,13,14- hexahydro-3,5- etheno[1,4]dioxacyclopentadecino[10,11- c:15,14-c′:6,7-c″]tripyrazole |
| 304 | 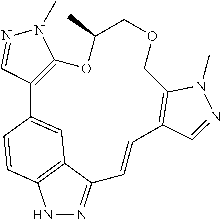 [SMILES: C[C@H]1COCc2c(cnn2C)/C=C/c2n[nH]c3ccc(cc23)-c2cnn(C)c2O1] | (10S,17E)-8,10,14-trimethyl- 2,8,10,11,13,14-hexahydro-3,5- etheno[1,4]dioxacyclopentadecino[10,11- c:15,14-c′:6,7-c″]tripyrazole |
| 305 | 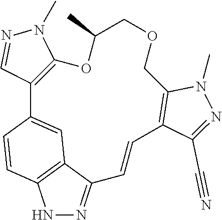 [SMILES: C[C@H]1COCc2c(c(C#N)nn2C)/C=C/c2n[nH]c3ccc(cc23)-c2cnn(C)c2O1] | (10S,17E)-8,10,14-trimethyl- 2,8,10,11,13,14-hexahydro-3,5- etheno[1,4]dioxacyclopentadecino[10,11- c:15,14-c′:6,7-c″]tripyrazole-16- carbonitrile |
| 306 | 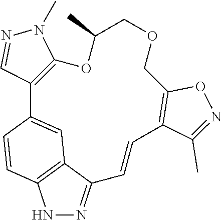 [SMILES: Cc1noc2c1/C=C/c1n[nH]c3ccc(cc13)-c1cnn(C)c1O[C@@H](C)COC2] | (10S,17E)-8,10,16-trimethyl-2,10,11,13- tetrahydro-8H-3,5- ethenodipyrazolo[3′,4′:10,11;4″,3″:14,15] [1,4]dioxacyclopentadecino[7,6- d][1,2]oxazole |
| 307 | 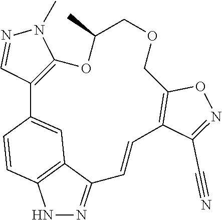 [SMILES: C[C@H]1COCc2onc(C#N)c2/C=C/c2n[nH]c3ccc(cc23)-c2cnn(C)c2O1] | (10S,17E)-8,10-dimethyl-2,10,11,13- tetrahydro-8H-3,5- ethenodipyrazolo[3′,4′:10,11;4″,3″:14,15] [1,4]dioxacyclopentadecino[7,6- d][1,2]oxazole-16-carbonitrile |
| 308 | 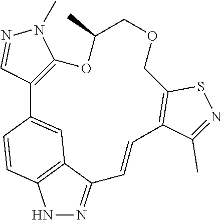 [SMILES: Cc1nsc2c1/C=C/c1n[nH]c3ccc(cc13)-c1cnn(C)c1O[C@@H](C)COC2] | (10S,17E)-8,10,16-trimethyl-2,10,11,13- tetrahydro-8H-3,5- ethenodipyrazolo[3′,4′:10,11;4″,3″:14,15] [1,4]dioxacyclopentadecino[7,6- d][1,2]thiazole |
| 309 | 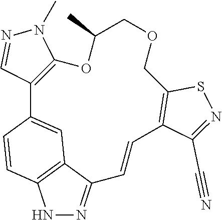 [SMILES: C[C@H]1COCc2snc(C#N)c2/C=C/c2n[nH]c3ccc(cc23)-c2cnn(C)c2O1] | (10S,17E)-8,10-dimethyl-2,10,11,13- tetrahydro-8H-3,5- ethenodipyrazolo[3′,4′:10,11;4″,3″:14,15] [1,4]dioxacyclopentadecino[7,6- d][1,2]thiazole-16-carbonitrile |
| 310 | 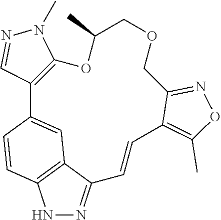 [SMILES: Cc1onc2c1/C=C/c1n[nH]c3ccc(cc13)-c1cnn(C)c1O[C@@H](C)COC2] | (10S,17E)-8,10,16-trimethyl-2,8,10,11- tetrahydro-13H-3,5- ethenodipyrazolo[3′,4′:10,11;4″,3″:14,15] [1,4]dioxacyclopentadecino[6,7- c][1,2]oxazole |
| 311 | 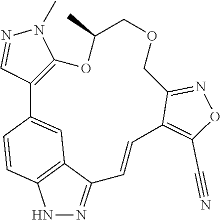 [SMILES: C[C@H]1COCc2noc(C#N)c2/C=C/c2n[nH]c3ccc(cc23)-c2cnn(C)c2O1] | (10S,17E)-8,10-dimethyl-2,8,10,11- tetrahydro-13H-3,5- ethenodipyrazolo[3′,4′:10,11;4″,3″:14,15] [1,4]dioxacyclopentadecino[6,7- c][1,2]oxazole-16-carbonitrile |
| 312 | 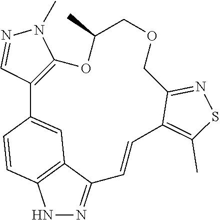 [SMILES: Cc1snc2c1/C=C/c1n[nH]c3ccc(cc13)-c1cnn(C)c1O[C@@H](C)COC2] | (10S,17E)-8,10,16-trimethyl-2,8,10,11- tetrahydro-13H-3,5- ethenodipyrazolo[3′,4′:10,11;4″,3″:14,15] [1,4]dioxacyclopentadecino[6,7- c][1,2]thiazole |
| 313 | 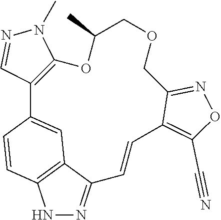 [SMILES: C[C@H]1COCc2noc(C#N)c2/C=C/c2n[nH]c3ccc(cc23)-c2cnn(C)c2O1] | (10S,17E)-8,10-dimethyl-2,8,10,11- tetrahydro-13H-3,5- ethenodipyrazolo[3′,4′:10,11;4″,3″:14,15] [1,4]dioxacyclopentadecino[6,7- c][1,2]oxazole-16-carbonitrile |
| 314 | 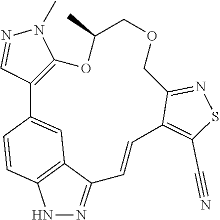 [SMILES: C[C@H]1COCc2nsc(C#N)c2/C=C/c2n[nH]c3ccc(cc23)-c2cnn(C)c2O1] | (10S,17E)-8,10-dimethyl-2,8,10,11- tetrahydro-13H-3,5- ethenodipyrazolo[3′,4′:10,11;4″,3″:14,15] [1,4]dioxacyclopentadecino[6,7- c][1,2]thiazole-16-carbonitrile |
| 315 | 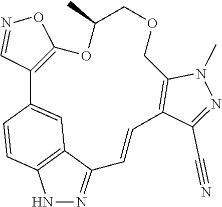 [SMILES: C[C@H]1COCc2c(c(C#N)nn2C)/C=C/c2n[nH]c3ccc(cc23)-c2cnoc2O1] | (10S,17E)-10,14-dimethyl-10,11,13,14- tetrahydro-2H-3,5- ethenodipyrazolo[4′,3′:13,14;4″,3″:9,10] [1,4]dioxacyclopentadecino[6,5- d][1,2]oxazole-16-carbonitrile |
| 316 | 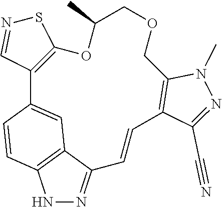 [SMILES: C[C@H]1COCc2c(c(C#N)nn2C)/C=C/c2n[nH]c3ccc(cc23)-c2cnsc2O1] | (10S,17E)-10,14-dimethyl-10,11,13,14- tetrahydro-2H-3,5- ethenodipyrazolo[4′,3′:13,14;4″,3″:9,10] [1,4]dioxacyclopentadecino[6,5- d][1,2]thiazole-16-carbonitrile |
| 317 | 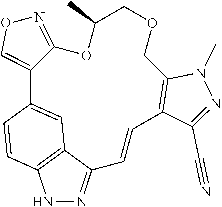 [SMILES: C[C@H]1COCc2c(c(C#N)nn2C)/C=C/c2n[nH]c3ccc(cc23)-c2conc2O1] | (10S,17E)-10,14-dimethyl-10,11,13,14- tetrahydro-2H-3,5- ethenodipyrazolo[4′,3′:13,14;4″,3″:9,10] [1,4]dioxacyclopentadecino[5,6- c][1,2]oxazole-16-carbonitrile |
| 318 | 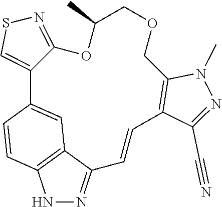 [SMILES: C[C@H]1COCc2c(c(C#N)nn2C)/C=C/c2n[nH]c3ccc(cc23)-c2csnc2O1] | (10S,17E)-10,14-dimethyl-10,11,13,14- tetrahydro-2H-3,5- ethenodipyrazolo[4′,3′:13,14;4″,3″:9,10] [1,4]dioxacyclopentadecino[5,6- c][1,2]thiazole-16-carbonitrile |
| 319 | 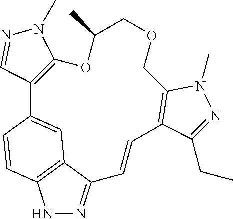 [SMILES: CCc1nn(C)c2c1/C=C/c1n[nH]c3ccc(cc13)-c1cnn(C)c1O[C@@H](C)COC2] | (10S,17E)-16-ethyl-8,10,14-trimethyl- 2,8,10,11,13,14-hexahydro-3,5- etheno[1,4]dioxacyclopentadecino[10,11- c:15,14-c′:6,7-c″]tripyrazole |
| 320 | 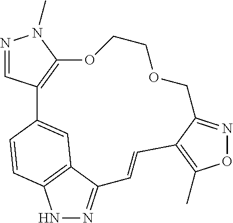 [SMILES: Cc1onc2c1/C=C/c1n[nH]c3ccc(cc13)-c1cnn(C)c1OCCOC2] | (17E)-8,16-dimethyl-2,8,10,11- tetrahydro-13H-3,5- ethenodipyrazolo[3′,4′:10,11;4″,3″:14,15] [1,4]dioxacyclopentadecino[6,7- c][1,2]oxazole |
| 321 | 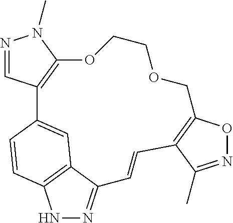 [SMILES: Cc1noc2c1/C=C/c1n[nH]c3ccc(cc13)-c1cnn(C)c1OCCOC2] | (17E)-8,16-dimethyl-2,10,11,13- tetrahydro-8H-3,5- ethenodipyrazolo[3′,4′:10,11;4″,3″:14,15] [1,4]dioxacyclopentadecino[7,6- d][1,2]oxazole |
| 322 | 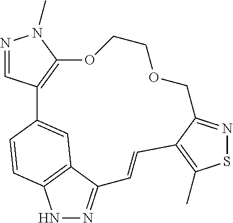 [SMILES: Cc1snc2c1/C=C/c1n[nH]c3ccc(cc13)-c1cnn(C)c1OCCOC2] | (17E)-8,16-dimethyl-2,8,10,11- tetrahydro-13H-3,5- ethenodipyrazolo[3′,4′:10,11;4″,3″:14,15] [1,4]dioxacyclopentadecino[6,7- c][1,2]thiazole |

and pharmaceutically acceptable salts thereof.

Those skilled in the art will recognize that the species listed or illustrated herein are not exhaustive, and that additional species within the scope of these defined terms may also be selected.

### ALTERNATIVE EMBODIMENTS

Certain compounds of the present disclosure are also described in the following alternative embodiments. The skilled person will recognize that the alternative embodiments described herein are consistent with embodiments described throughout the disclosure. The alternative embodiments describe certain aspects of the disclosure that are within the general teaching of the disclosure, and are described in a similar manner.

In some embodiments, the disclosure provides a compound of the formula IA, or a pharmaceutically acceptable salt thereof,

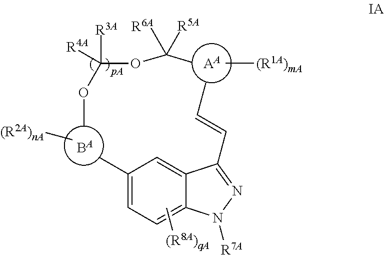

wherein R^{1A}, R^{2A}, R^{3A}, R^{4A}, R^{5A}, R^{6A}, R^{7A}, R^{8A}, A^{A}, B^{A}, m^{A}, n^{A}, p^{A} and q^{A} are as described herein.

In some embodiments, the disclosure provides a compound of the formula IIA, or a pharmaceutically acceptable salt thereof,

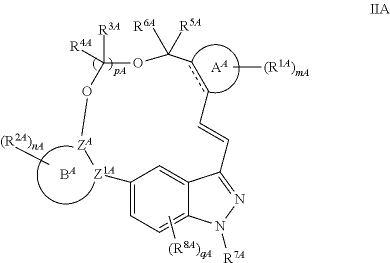

wherein R^{1A}, R^{2A}, R^{3A}, R^{4A}, R^{5A}, R^{6A}, R^{7A}, R^{8A}, A^{A}, B^{A}, Z^{A}, Z^{1A}, m^{A}, n^{A}, p^{A}, q^{A}, and “ [symbol] ” are as described herein.

In some embodiments, the disclosure provides a compound of the formula IIIA, or a pharmaceutically acceptable salt thereof,

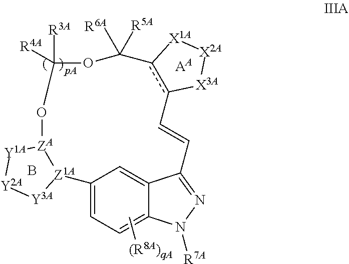

wherein R^{3A}, R^{4A}, R^{5A}, R^{6A}, R^{7A}, R^{8A}, A^{A}, B^{A}, X^{1A}, X^{2A}, X^{3A}, Z^{A}, Z^{1A}, Y^{1A}, Y^{2A}, Y^{3A}, p^{A}, q^{A}, and “ [symbol] ” are as described herein.

In some embodiments, the disclosure provides a compound of the formula IVA, or a pharmaceutically acceptable salt thereof,

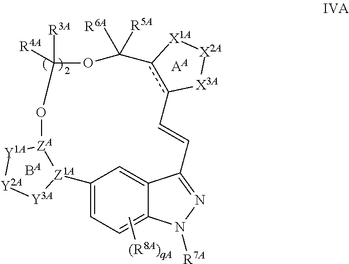

wherein R^{3A}, R^{4A}, R^{5A}, R^{6A}, R^{7A}, R^{8A}, A^{A}, B^{A}, X^{1A}, X^{2A}, X^{3A}, Z^{A}, Z^{1A}, Y^{1A}, Y^{2A}, Y^{3A}, and q^{A} are as described herein.

In further aspects, the disclosure relates to a pharmaceutical composition comprising at least one compound of Formula (IA)-(IVA) or a pharmaceutically acceptable salt thereof. Pharmaceutical compositions according to the disclosure may further comprise a pharmaceutically acceptable excipient.

In further aspects, the disclosure relates to a compound of Formula (IA)-(IVA), or a pharmaceutically acceptable salt thereof, for use as a medicament.

In further aspects, the disclosure relates to a method of treating disease, such as cancer comprising administering to a subject in need of such treatment an effective amount of at least one compound of Formula (IA)-(IVA), or a pharmaceutically acceptable salt thereof.

In further aspects, the disclosure relates to use of a compound of Formula (IA)-(IVA), or a pharmaceutically acceptable salt thereof, in the preparation of a medicament for the treatment of disease, such as cancer, and the use of such compounds and salts for treatment of such diseases.

In further aspects, the disclosure relates to a method of inhibiting a ALK, comprising contacting a cell comprising one or more of ALK with an effective amount of at least one compound of Formula (IA)-(IVA), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula IA

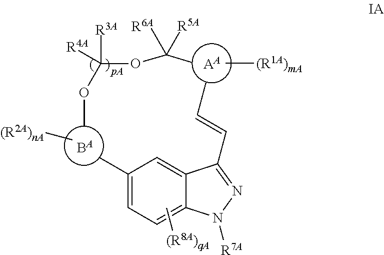

wherein
ring A^{A} and ring B^{A} are each independently a 5-membered heteroarylene;
each R^{1A}, R^{2A}, and R^{8A} when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{1A} and R^{5A} or R^{6A}, can be taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R^{1A} and R^{5A} or R^{6A} are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{1A}, R^{4A}, R^{5A}, and R^{6A}, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R^{3A}, R^{4A}, R^{5A}, and R^{6A}, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R^{3A}, R^{4A}, R^{5A}, and R^{6A} are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R^{7A} is independently H, deuterium, —C(O)R^{c}, —C(O)OR^{c}, —C(O)NR^{c}R^{d}, —S(O)₂NR^{c}R^{d}, —P(O)₂R^{c}R^{d}, —P(O)₂NR^{c}R^{d}, or —P(O)₂OR^{c};
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{e} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m^{A} is 0, 1, 2, or 3;
n^{A} is 0, 1, 2, or 3;
p^{A} is 2, 3, or 4; and
q^{A} is 0, 1, or 2;
or a pharmaceutically acceptable salt thereof.

2. The compound of embodiment 1, or a pharmaceutically acceptable salt thereof, having the formula HA

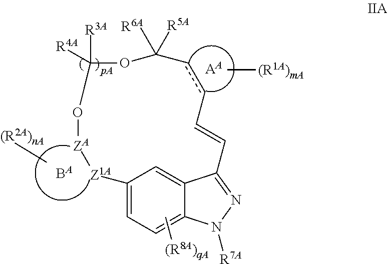

wherein
“ [symbol] ” is optionally a carbon-carbon single bond or a carbon-carbon double bond;

Z^{A} is

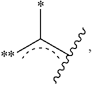

wherein * is a point of covalent attachment to ether, ** is a point of covalent attachment to Z^{1A}, “ [symbol] ” represents a point of covalent attachment to a ring atom of ring B^{A}, and “ [symbol] ” represents the condition that between the points of attachment ** and “ [symbol] ”, one bond is a single bond and one bond is a double bond; Z^{1A} is

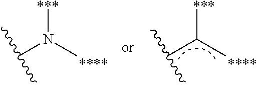

wherein *** is a point of covalent attachment to indazole, **** is a point of covalent attachment to Z^{A}, “ [symbol] ” represents a point of covalent attachment to a ring atom of ring B^{A}, and “ [symbol] ” indicates the condition that between the points of attachment **** and “ [symbol] ” one bond is a single bond and one bond is a double bond; and ring B^{A} is a 5-membered heteroarylene.

3. The compound of embodiment 1 or 2, or a pharmaceutically acceptable salt thereof, having the formula IIIA

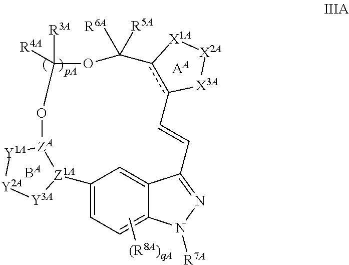

wherein
X^{1A}, X^{2A}, and X^{3A} are each independently —O—, —S—, ═C(H)—, ═C(R^{1A})—, —N(H)—, —N(R^{1A})— or ═N— and ring A^{A} is a 5-membered heteroarylene, provided that at least one of X^{1A}, X^{2A}, and X^{3A} is not ═C(H)—, or ═C(R^{1A})—;
Y^{1A}, Y^{2A} and Y^{3A} are each independently —O—, —S—, ═C(H)—, ═C(R^{2A})—, —N(H)—, —N(R^{2A})— or ═N— and ring B is a 5-membered heteroarylene, provided that at least one of Y^{1A}, Y^{2A}, and Y^{3A} is not ═C(H)—, or ═C(R^{2A})—; and
“ [symbol] ” is optionally a carbon-carbon single bond or a carbon-carbon double bond;
Z^{A} is

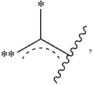

wherein * is a point of covalent attachment to ether, ** is a point of covalent attachment to Z^{1A}, “ [symbol] ” represents a point of covalent attachment to a ring atom of ring B^{A}, and “ [symbol] ” represents the condition that between the points of attachment ** and “ [symbol] ”, one bond is a single bond and one bond is a double bond; Z^{1A} is

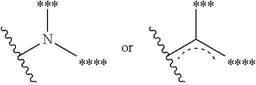

or wherein *** is a point of covalent attachment to indazole, **** is a point of covalent attachment to Z^{A}, “ [symbol] ” represents a point of covalent attachment to a ring atom of ring B^{A}, and “ [symbol] ” indicates the condition that between the points of attachment **** and “ [symbol] ”, one bond is a single bond and one bond is a double bond; and ring B^{A} is a 5-membered heteroarylene.

4. The compound of any one of embodiments 1 to 3, or a pharmaceutically acceptable salt thereof, having the formula IVA

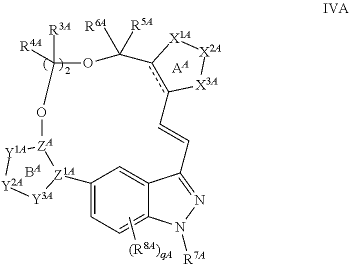

wherein
“ [symbol] ” is optionally a carbon-carbon single bond or a carbon-carbon double bond;
Z^{A} is

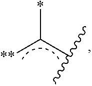

wherein * is a point of covalent attachment to ether, ** is a point of covalent attachment to Z^{1A}, “ [symbol] ” represents a point of covalent attachment to a ring atom of ring B^{A}, and “ [symbol] ” represents the condition that between the points of attachment ** and “ [symbol] ” one bond is a single bond and one bond is a double bond; Z^{1A} is

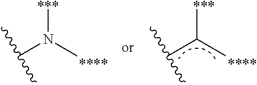

or wherein *** is a point of covalent attachment to indazole, **** is a point of covalent attachment to Z^{A}, “ [symbol] ” represents a point of covalent attachment to a ring atom of ring B^{A}, and “ [symbol] ” indicates the condition that between the points of attachment **** and “ [symbol] ” one bond is a single bond and one bond is a double bond; and ring B^{A} is a 5-membered heteroarylene.

5. The compound of any one of embodiments 1 to 3, wherein
X^{2A}, is —O—, —S—, —N(H)—, —N(R^{1A})— or ═N—, X^{1A} and X^{3A} are each independently —O—, —S—, ═C(H)—, ═C(R^{1A})—, —N(H)—, —N(R^{1A})— or ═N—, and ring A^{A} is a 5-membered heteroarylene; and
Y^{2A} is —O—, —S—, —N(H)—, —N(R^{2A})— or ═N—, Y^{1A} and Y^{3A} are each independently —O—, —S—, ═C(H)—, ═C(R^{2A})—, —N(H)—, —N(R^{2A})— or ═N—, and ring B^{A} is a 5-membered heteroarylene.
6. The compound of any one of embodiments 1 to 5, or a pharmaceutically acceptable salt thereof, wherein
ring A^{A} is a 5-membered heteroarylene selected from the group consisting of

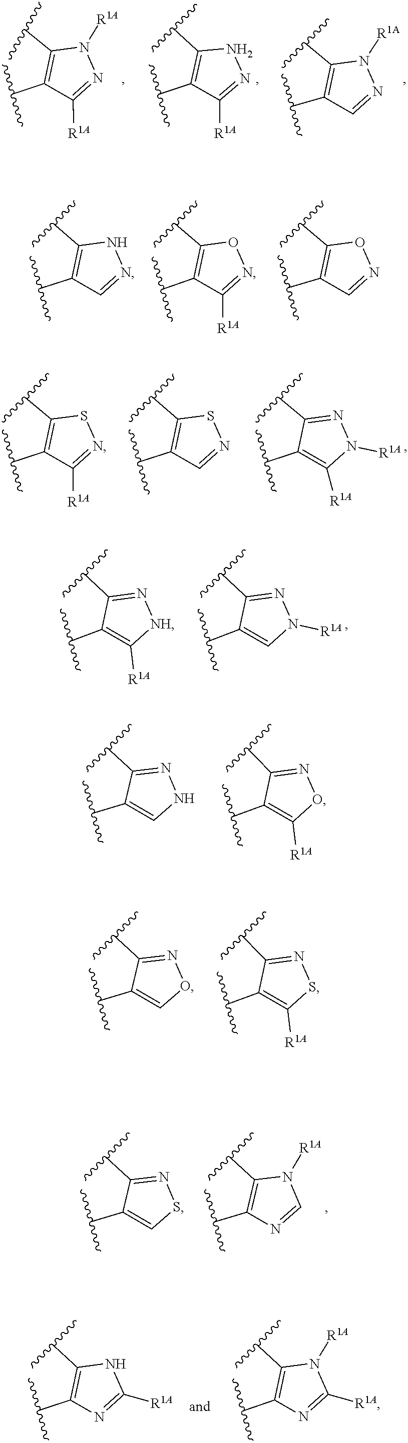

wherein each “ [symbol] ” represents a point of covalent attachment, and each R^{1A} is independently as described herein.

7. The compound of any one of embodiments 1 to 6, or a pharmaceutically acceptable salt thereof, wherein ring A^{A} is a 5-membered heteroarylene selected from the group consisting of

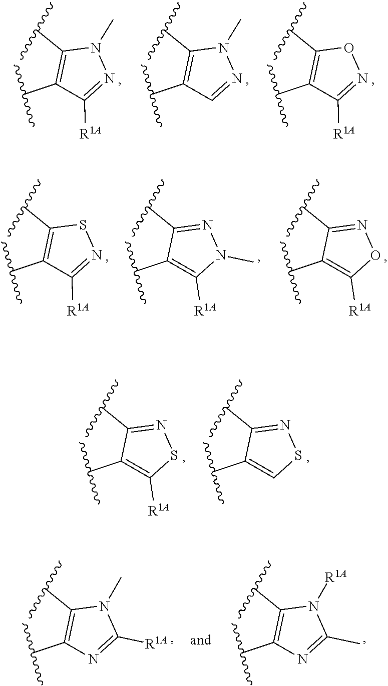

wherein each “ [symbol] ” represents a point of covalent attachment, and R^{1A} is as described herein.

8. The compound of any one of embodiments 1 to 6, or a pharmaceutically acceptable salt thereof, wherein ring A^{A} is a 5-membered heteroarylene selected from the group consisting of

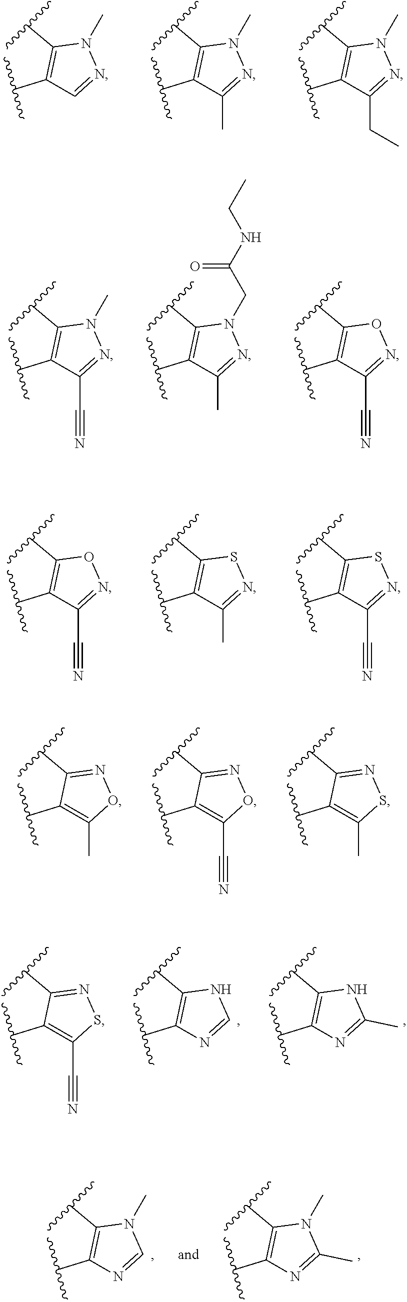

wherein each “ [symbol] ” represents a print of covalent attachment.

9. The compound of any one of the preceding embodiments, or a pharmaceutically acceptable salt thereof, wherein ring B^{A} is a 5-membered heteroarylene selected from the group consisting of

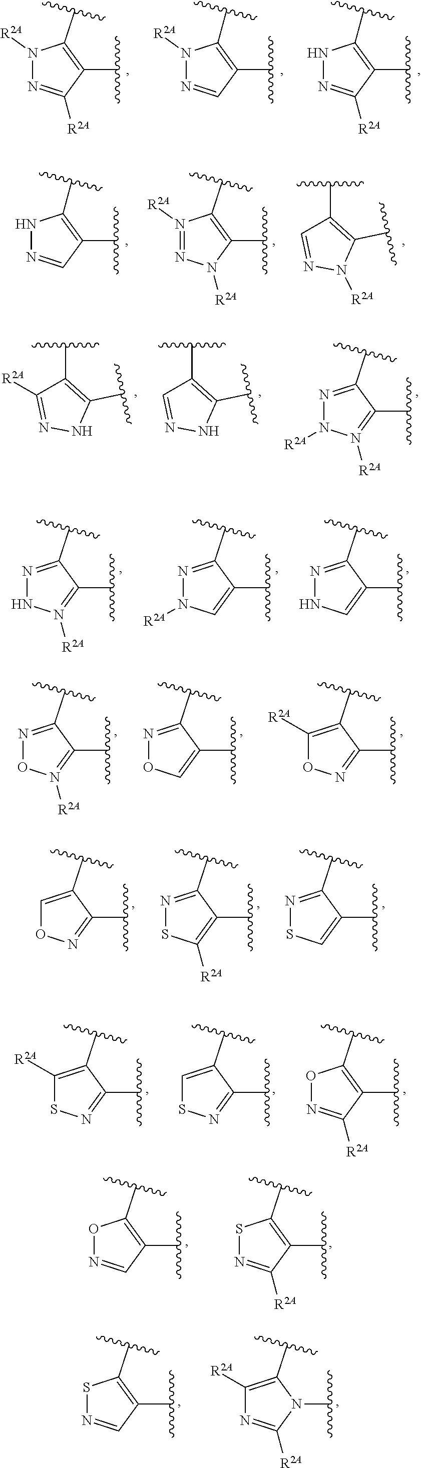

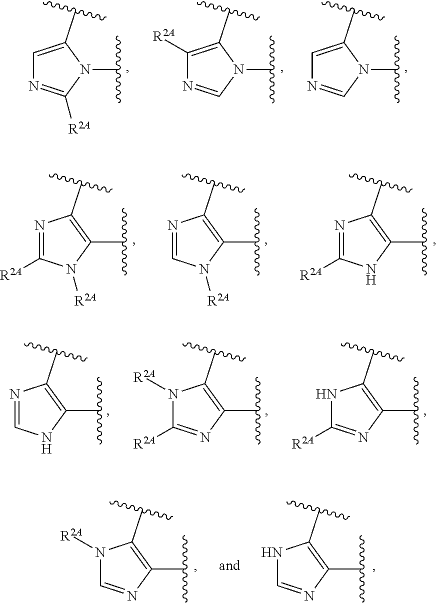

wherein each “ [symbol] ” represents a point of covalent attachment, and each R² is independently as described herein.

10. The compound of any one of the preceding embodiments, or a pharmaceutically acceptable salt thereof, wherein ring B^{A} is a 5-membered heteroarylene selected from the group consisting of

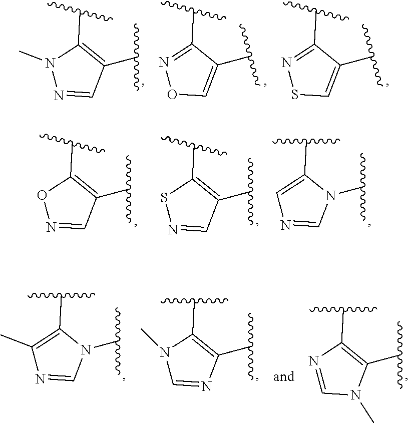

wherein each “ [symbol] ” represents a point of covalent attachment.

11. The compound of any one of the preceding embodiments, or a pharmaceutically acceptable salt thereof, wherein

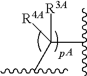

is an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, and each “ [symbol] ” represents a point of covalent attachment.

12. The compound of any one of the preceding embodiments, or a pharmaceutically acceptable salt thereof, wherein one R^{3A} is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two or R^{3A} and/or R^{4A}, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R³ and/or R^{4A} are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and any remaining R^{3A} and R^{4A} are H.

13. The compound of any one of the preceding embodiments, or a pharmaceutically acceptable salt thereof, wherein one R^{3A} is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{c}, —P(O)₂OR^{c}, —CN, or —NO₂, and any remaining R^{3A} and R^{4A} are H.

14. The compound of any one of the preceding embodiments, or a pharmaceutically acceptable salt thereof, wherein one R^{3A} is C₁-C₆ alkyl.

15. The compound of any one of the preceding embodiments, or a pharmaceutically acceptable salt thereof, wherein one R^{3A} is methyl, and any remaining R^{3A} and R^{4A} are H.

16. The compound of any one of the preceding embodiments, or a pharmaceutically acceptable salt thereof, wherein R^{5A} and R^{6A} are H

17. The compound of any one of the preceding embodiments, or a pharmaceutically acceptable salt thereof, wherein R^{7A} is H.

18. The compound of any one of the preceding claims, or a pharmaceutically acceptable salt thereof, wherein q^{A} is 0.

19. The compound of embodiment 1, selected from the group consisting of

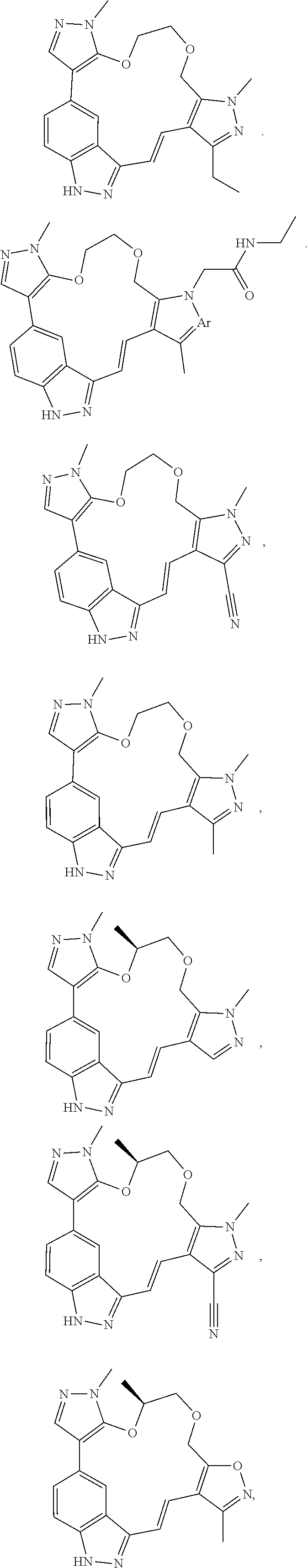

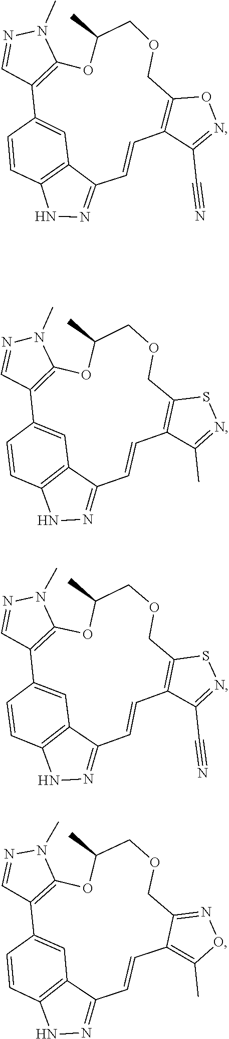

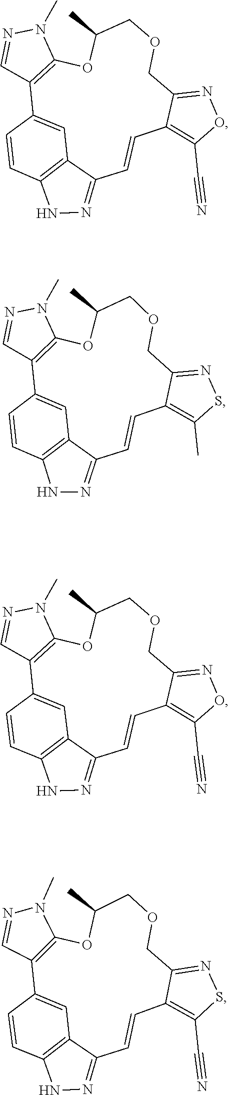 [SMILES: C[C@H]1COCc2noc(C#N)c2/C=C/c2n[nH]c3ccc(cc23)-c2cnn(C)c2O1 ; Cc1snc2c1/C=C/c1n[nH]c3ccc(cc13)-c1cnn(C)c1O[C@@H](C)COC2 ; C[C@H]1COCc2noc(C#N)c2/C=C/c2n[nH]c3ccc(cc23)-c2cnn(C)c2O1 ; C[C@H]1COCc2nsc(C#N)c2/C=C/c2n[nH]c3ccc(cc23)-c2cnn(C)c2O1]

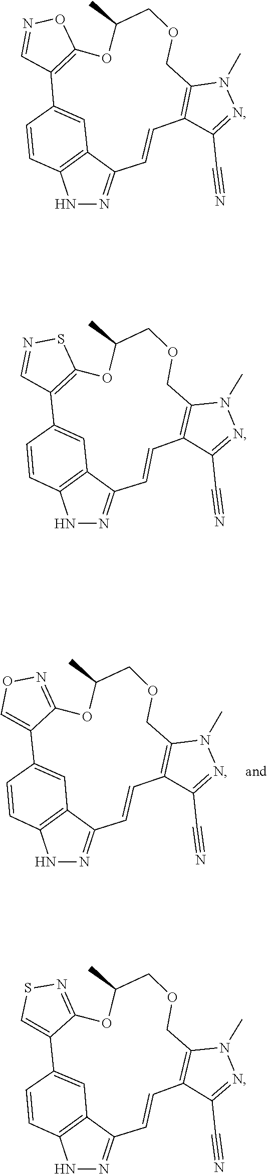

or a pharmaceutically acceptable salt thereof.

20. A pharmaceutical composition comprising a compound of any one of the preceding embodiments, and optionally one or more excipients.

21. A method of treating disease in a subject comprising, administering a therapeutically effective amount of a compound of any one of embodiments 1 to 19, or a pharmaceutical composition of embodiment 20.

22. A compound according to any one of embodiments 1 to 19, for use in a method of treating disease in a subject.

23. Use of a compound according to any one of embodiments 1 to 19 in the manufacture of a medicament for the treatment of disease in a subject.

In some embodiments, Ring A^{A} is a 5-membered heteroarylene.

In some embodiments, Ring A^{A} is a 5-membered heteroarylene, wherein each R^{1A} when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{e}, —OS(O)₂R^{e}, —OS(O)NR^{c}R^{d}, —OS(O)₂NR^{c}R^{d}, —SR^{e}, —S(O)R^{c}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{e}, —CN, or —NO₂; each R^{1A} and R^{5A} or R^{6A}, can be taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R^{1A} and R^{5A} or R^{6A} are taken together is independently optionally substituted by OR^{e}, OC(O)R^{e}, OC(O)NR^{e}R^{f}, —OS(O)R^{e}, OS(O)₂R^{e}, OS(O)NR^{e}R^{f}, —OS(O)₂NR^{e}R^{f}, —SR^{e}, S(O)R^{e}, —S(O)₂R^{e}, S(O)NR^{e}R^{f}, S(O)₂NR^{e}R^{f}, NR^{e}R^{f}, NR^{e}C(O)R^{f}, —NR^{e}C(O)OR^{f}, NR^{e}C(O)NR^{e}R^{f}, NR^{e}S(O)R^{f}, —NR^{e}S(O)₂R^{f}, NR^{e}S(O)NR^{e}R^{f}, NR^{e}S(O)₂NR^{e}R^{f}, —C(O)R^{e}, C(O)OR^{e}, C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or NO₂.

In some embodiments, an R^{1A} of Ring A^{A} and an R^{5A} or R^{6A}, can be taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R^{1A} and R^{5A} or R^{6A} are taken together is independently optionally substituted by OR^{e}, OC(O)R^{e}, OC(O)NR^{e}R^{f}, —OS(O)R^{e}, OS(O)₂R^{e}, OS(O)NR^{e}R^{f}, OS(O)₂NR^{e}R^{f}, —SR^{e}, S(O)R^{e}, —S(O)₂R^{e}, S(O)NR^{e}R^{f}, —S(O)₂NR^{e}R^{f}, NR^{e}R^{f}, —NR^{e}C(O)R^{f}, NR^{e}C(O)OR^{f}, NR^{e}C(O)NR^{e}R^{f}, NR^{e}S(O)R^{f}, —NR^{e}S(O)₂R^{f}, NR^{e}S(O)NR^{e}R^{f}, —NR^{e}S(O)₂NR^{e}R^{f}, C(O)R^{e}, C(O)OR^{e}, C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, P(O)OR^{e}, —P(O)₂OR^{e}, —CN, or NO₂.

In some embodiments, Ring A^{A} is pyrazolylene, isoxazolylene, isothiazolylene, imidazolylene wherein each is independently optionally substituted by 1, 2, or 3 R^{1A} (m of R^{1A}) each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{c}, —OC(O)NR^{c}R^{d}, —OC(═N)NR^{c}R^{d}, —OS(O)R^{c}, —OS(O)₂R^{e}, —OS(O)NR^{c}R^{d}, —OS(O)₂NR^{c}R^{d}, —SR^{e}, —S(O)R^{e}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{c}, —CN, or —NO₂.

In some embodiments, Ring A^{A} is of the formula

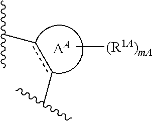

wherein “ [symbol] ” optionally a carbon-carbon single bond or a carbon-carbon double bond, each “ [symbol] ” represents a point of covalent attachment, and R^{1A} and m^{A} are as described herein. In some embodiments, Ring A^{A} is of the formula

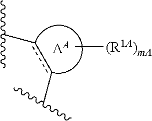

wherein “ [symbol] ” is optionally a carbon-carbon single bond or a carbon-carbon double bond, each “ [symbol] ” represents a point of covalent attachment, Ring A^{A} is a 5-membered heteroarylene, and R^{1A} and m^{A} are as described herein.

In some embodiments, Ring A^{A} is of the formula

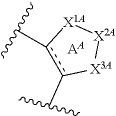

wherein “ [symbol] ” is optionally a carbon-carbon single bond or a carbon-carbon double bond, each “ [symbol] ” represents a point of covalent attachment, X², is —O—, —S—, —N(H)—, —N(R^{1A})— or ═N—, X^{1A} and X^{3A} are each independently —O—, —S—, ═C(H)—, ═C(R^{1A})—, —N(H)—, N(R⁵)— or ═N—, and ring A^{A} is a 5-membered heteroarylene, provided that at least one of X^{1A}, X^{2A}, and X^{3A} is not ═C(H)—, or ═C(R¹)—, Ring A^{A} is a 5-membered heteroarylene, and R^{1A} and m are as described herein.

In some embodiments, m^{A} is 0, 1, 2, or 3. In some embodiments, m^{A} is 0, 1, or 2. In some embodiments, mA is 0 or 1. In some embodiments, m^{A} is 0. In some embodiments, m is 1. In some embodiments, m^{A} is 2. In some embodiments, m^{A} is 3

In some embodiments, ring A^{A} is a 5-membered heteroarylene selected from the group consisting of

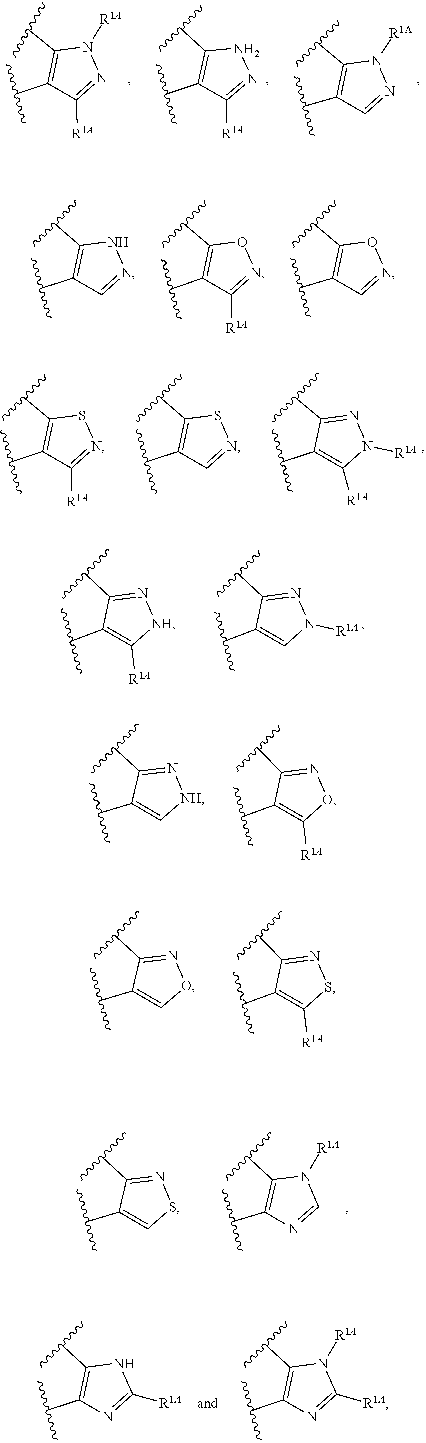

wherein each “ [symbol] ” represents a point of covalent attachment, and each RIA is independently as described herein.

In some embodiments, ring A^{A} is a 5-membered heteroarylene selected from the group consisting of

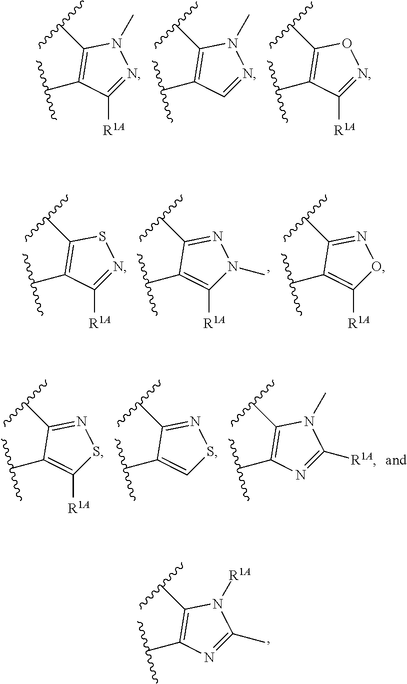

wherein each “ [symbol] ” represents a point of covalent attachment, and R^{1A} is as described herein.

In some embodiments, R^{1A} is C₁-C₆ alkyl wherein each hydrogen atom in C₁-C₆ alkyl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A^{A} is a 5-membered heteroarylene selected from the group consisting of

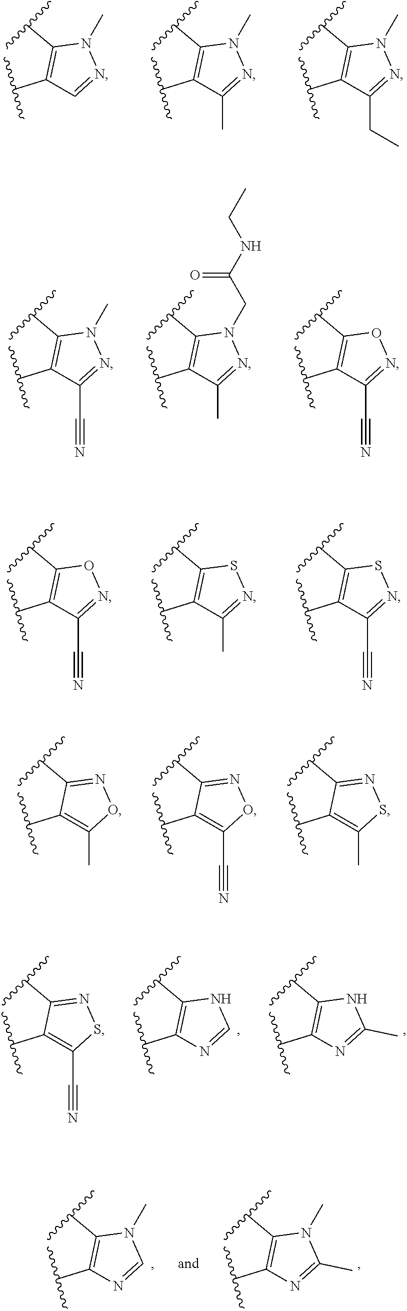

wherein each “ [symbol] ” represents a point of covalent attachment.

In some embodiments, Ring B^{A} is 5-membered heteroarylene.

In some embodiments, Ring B^{A} is a 5-membered heteroarylene, wherein each R^{2A} when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, Ring B^{A} is of the formula

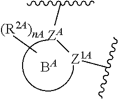

wherein each “ [symbol] ” represents a point of covalent attachment, and R^{2A} and n^{A} are as described herein,
wherein Z^{A} is

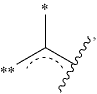 [SMILES: *C(C)C]

wherein * is a point of covalent attachment to ether, ** is a point of covalent attachment to Z^{A}, “ [symbol] ” represents a point of covalent attachment to a ring atom of ring B^{A}, and “ [symbol] ” represents the condition that between the points of attachment ** and “ [symbol] ”, one bond is a single bond and one bond is a double bond; Z^{1A} is

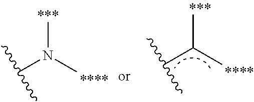

wherein *** is a point of covalent attachment to indazole, **** is a point of covalent attachment to Z^{A}, “ [symbol] ” represents a point of covalent attachment to a ring atom of ring B^{A}, and “ [symbol] ” indicates the condition that between the points of attachment **** and “ [symbol] ”, one bond is a single bond and one bond is a double bond.

In some embodiments, Ring B^{A} is of the formula

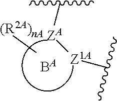

wherein each “ [symbol] ” represents a point of covalent attachment, Ring B^{A} is a 5-membered heteroarylene and Z^{A}, Z^{1A}, R^{2A} and n^{A} are as described herein.

In some embodiments, Ring B^{A} is of the formula

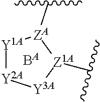

Wherein each “ [symbol] ” represents a point of covalent attachment, Y^{1A}, Y^{2A} and Y^{3A} are each independently —O—, —S—, ═C(H)—, ═C(R^{2A})—, —N(H)—, —N(R^{2A})— or ═N—, and ring B is a 5-membered heteroarylene, provided that at least one of Y^{1A}, Y^{2A} and Y^{3A} is not ═C(H)—, or ═C(R^{2A})—, and R^{2A}, Z^{A}, Z^{1A}, and n^{A} are as described herein.

In some embodiments, Z^{1A} is N. In some embodiments, Z^{1A} is C.

In some embodiments, Ring B^{A} is pyrazolylene, isoxazolylene, isothiazolylene, imidazolylene wherein each is optionally substituted with 1, 2, or 3 R^{2A} (n^{A} of R^{2A}), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, n^{A} is 0, 1, 2, or 3. In some embodiments, n^{A} is 0, 1, or 2. In some embodiments, n^{A} is 0 or 1. In some embodiments, n^{A} is 0. In some embodiments, n^{A} is 1. In some embodiments, n^{A} is 2. In some embodiments, n^{A} is 3.

In some embodiments, ring BA is a 5-membered heteroarylene selected from the group consisting of

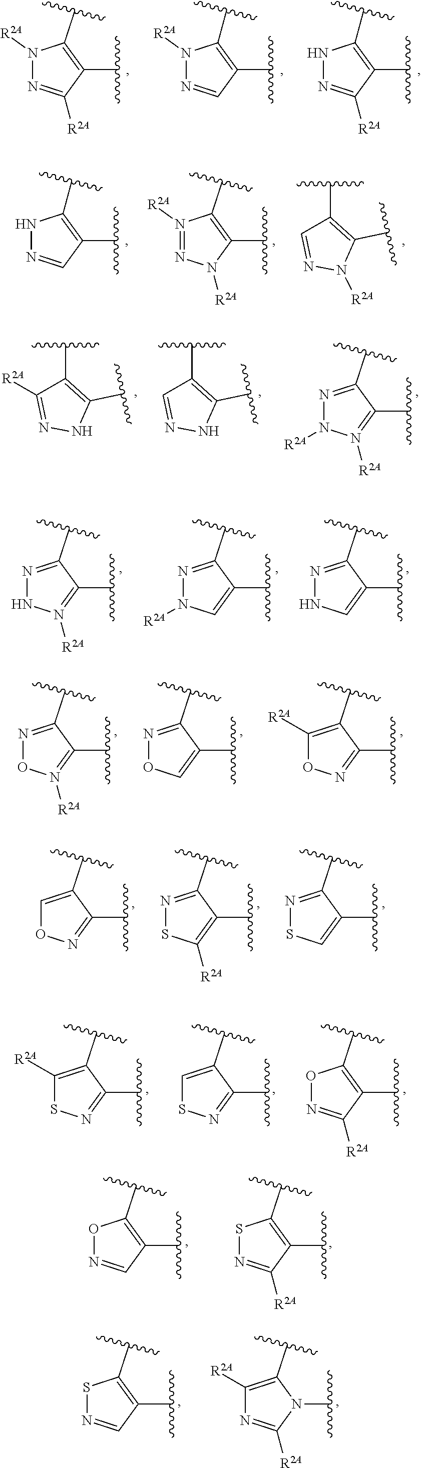

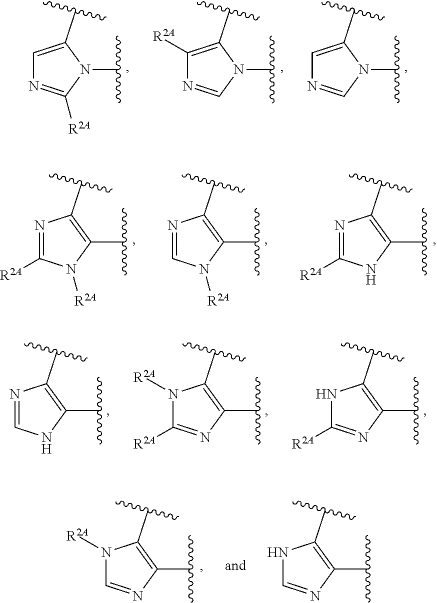

wherein each “ [symbol] ” represents a point of covalent attachment, and each R^{2A} is independently as described herein.

In some embodiments, ring B^{A} is a 5-membered heteroarylene selected from the group consisting of

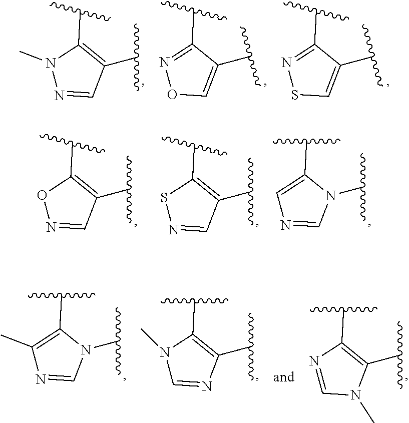

wherein each “ [symbol] ” represents a point of covalent attachment.

In some embodiments, R^{7A} is independently H, deuterium, —C(O)R^{c}, —C(O)OR^{c}, —C(O)NR^{c}R^{d}, —S(O)₂NR^{c}R^{d}, —P(O)₂R^{c}R^{d}, —P(O)₂NR^{c}R^{d}, or —P(O)₂OR^{c}.

In some embodiments, R^{7A} is independently H.

In some embodiments, indazole is optionally substituted with 0, 1, or 2 R^{8A} (q of R^{8A}), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, each R^{8A} when present, is independently hydrogen.

In some embodiments, q^{A} is 0, 1, or 2. In some embodiments, q^{A} is 0 or 1. In some embodiments, q^{A} is 0. In some embodiments, q^{A} is 1. In some embodiments, q^{A} is 2.

In some embodiments,

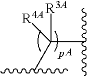

is an ethylene, propylene, or butylene, wherein each hydrogen atom in ethylene, propylene, and butylene is independently optionally substituted by deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, and each “ [symbol] ” represents a point of covalent attachment.

In some embodiments, p^{A} is 2, 3, or 4. In some embodiments, p^{A} is 2 or 3. In some embodiments, p^{A} is 2. In some embodiments, p^{A} is 3. In some embodiments, p^{A} is 4.

In some embodiments, one R^{3A} is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}e, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or two or R^{3A} and/or R^{4A}, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R^{3A} and/or R^{4A} are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{c}C(O)R^{f}, —NR^{c}C(O)OR^{f}, —NR^{c}C(O)NR^{e}R^{f}, —NR^{c}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and any remaining R³ and R⁴ are H.

In some embodiments, one R^{3A} is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, and any remaining R³ and R⁴ are H.

In some embodiments, one R^{3A} is C₁-C₆ alkyl. In some embodiments, one R^{3A} is methyl, and any remaining R^{3A} and R^{4A} are H.

In some embodiments, R^{5A} and R^{6A} are H.

In some embodiments, the disclosure provides a compound selected from the group consisting of
(17E)-16-ethyl-8,14-dimethyl-2,8,10,11,13,14-hexahydro-3,5-etheno[1,4]dioxacyclopentadecino[10,11-c:15,14-c′:6,7-c″ ]tripyrazole;
2-[(17E)-8,16-dimethyl-2,10,11,13-tetrahydro-3,5-etheno[1,4]dioxacyclopentadecino[10,11-c:15,14-c′:6,7-c″ ]tripyrazol-14(8H)-yl]-N-ethylacetamide;
(17E)-8,14-dimethyl-2,8,10,11,13,14-hexahydro-3,5-etheno[1,4]dioxacyclopentadecino[10,11-c:15,14-c′:6,7-c″ ]tripyrazole-16-carbonitrile;
(17E)-8,14,16-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″ ]tripyrazole-16-carbonitrile;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]oxazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole-16-carbonitrile;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]thiazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]thiazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]oxazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10,16-trimethyl-2,8,10,11-tetrahydro-13H-3,5-ethenodipyrazolo[3′,4′:10,11;4″,3″:14,15][1,4]dioxacyclopentadecino[6,7-c][1,2]thiazole;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]oxazole-16-carbonitrile;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]thiazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]thiazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]thiazole-16-carbonitrile;
or a pharmaceutically acceptable salt thereof.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, myelodysplastic syndromes (MDS), chronic myelomonocytic leukemia, myelodysplastic syndromes (MDS), chronic myelomonocytic leukemia (CML), acute myeloid leukemia (AML), congenital mesoblastic nephroma, congenital fibrosarcomas, Ph-like acute lymphoblastic leukemia, thyroid carcinoma, skin cutaneous melanoma, head and neck squamous cell carcinoma, pediatric glioma, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma.

In one aspect, the compounds and pharmaceutical compositions of the disclosure specifically target EGFR. Thus, these compounds and pharmaceutical compositions can be used to prevent, reverse, slow, or inhibit diseases, such as cancers driven by the activity of EGFR. In some embodiments, the compounds described herein can target EGFR in a oncogenic driver mutation, such as L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S. In some embodiments, the compounds described herein can target EGFR having one or more resistance mutations, such as such as resistance mutations. In some embodiments, the compounds described herein can inhibit EGFR in a oncogenic driver mutation, such as L858R, Del19, Δ746-750, Δ746-750/T790M, Δ746-750/C979S, L858R/T790M, Del19/T790M, L858R/C979S, Del19/C979S, L858R/T790M/C979S, and Δ746-750/T790M/C979S, and/or other emerging and established resistance mutations, while maintaining good selectivity over wild-type EGFR. In some embodiments, methods of treating a target cancer are described.

In one aspect, the compounds and pharmaceutical compositions of the disclosure specifically target PIM kinases. In some embodiments, the compounds described herein can target PIM kinase activity to overcome resistance mechanisms of chemotherapy, radiotherapy, anti-angiogenic therapies and targeted therapies. In some embodiments, methods of treating a target cancer, such as AML, are described.

In one aspect, the compounds and pharmaceutical compositions of the disclosure specifically target CLK kinases. In some embodiments, the compounds described herein can target CLK kinase activity to treat diseases, such as cancers, through modulation of pre-mRNA splicing via inhibition of CLK kinase activity. In some embodiments, methods of treating a target cancer, such as myelodysplastic syndromes (MDS), chronic myelomonocytic leukemia, AML, lung cancer, breast cancer, and pancreatic cancer are described.

In some embodiments, compounds as described herein can be useful in connection with the treatment of diseases, such as cancer, by inhibiting one or more of EGFR, including oncogenic driver mutations as described herein and/or resistance mutations, aberrant PIM kinases, and/or aberrant CLK kinases.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to a mutation of EGFR, PIM, and/or CLK as described herein.

In treatment methods according to the disclosure, an “effective amount” means an amount or dose sufficient to generally bring about the desired therapeutic benefit in subjects needing such treatment, such as those described herein having a disease, such as cancer, including those associated with EGFR, including oncogenic driver mutations as described herein and/or resistance mutations, aberrant PIM kinases, and/or aberrant CLK kinases. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g. crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(XII) and (IA-IVA).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′-Bis(diphenylphosphino)ferrocene]di- |
| | chloropalladium(II) |
| MeCN (ACN) | Acetonitrile |
| Pd₂(dba)₃ | Tris(dibenzylideneacetone)dipalladium(0) |
| Hunig′s Base | N,N-diisopropylethylamine |
| TBAF | Tert butyl ammonium fluoride |
| PPh_{₃} | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| AcOH | Acetic Acid |
| DMAc | N, N-Dimethylformamide |
| BPD | 4,4,5,5-tetramethyl-2-(4,4,5,5-tetramethyl- |
| | 1,3,2-dioxaborolan-2-yl)-1,3,2-dioxaborolane |
| MTBE | Methy tert-Butyl Ether |
| AIBN | 2,2'-Azobis(2-methylpropionitrile) |
| NBS | N-bromosuccinimide |
| NIS | N-iodosuccinimide |
| TBAI | Tetrabutylammonium iodide |
| DHP | Dihydropyran |
| TBS | tert-Butyldimethylsilyl |
| TIPS | Triisopropylsilyl |
| THP | Tetrahydropyran |
| B₂pin₂ | Bis(pinacolato)diboron |

The compounds described herein can be prepared via conventional chemistry or following the general methods as shown below.

### Preparation of 4-bromo-1-methyl-5-[2-(2-vinylphenoxy)ethoxymethyl]pyrazole (A-2-1)

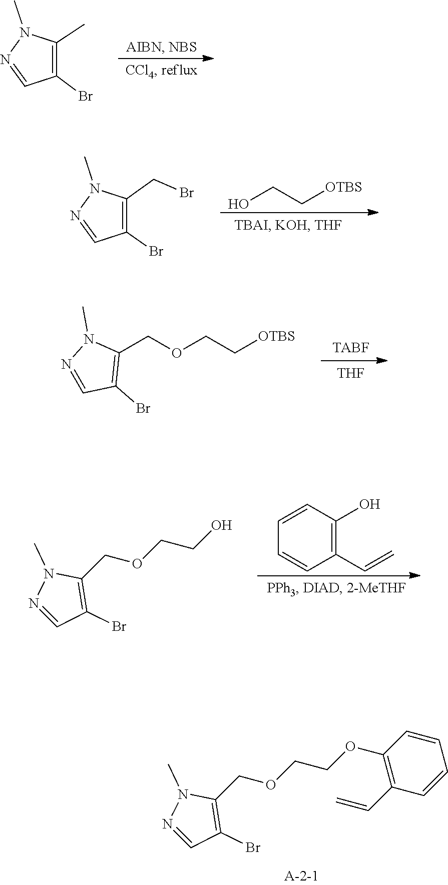

Step 1. To a solution of 4-bromo-1,5-dimethyl-pyrazole (15.0 g, 85.7 mmol, 1 eq) in CCl₄ (200 mL) was added AIBN (1.41 g, 8.57 mmol, 0.1 eq) and NBS (15.2 g, 85.7 mmol, 1 eq). The resulting mixture was stirred at 60° C. for 12 h. On completion, the mixture was concentrated. The residue was purified by column chromatography to give 4-bromo-5-(bromomethyl)-1-methyl-pyrazole (20 g, 78.76 mmol, 91.91% yield) as a white solid. 1H NMR (400 MHz, DMSO-d₆) δ=7.56 (s, 1H), 4.75 (s, 2H), 3.87 (s, 3H).

Step 2. To a solution of 4-bromo-5-(bromomethyl)-1-methyl-pyrazole (20.0 g, 78.8 mmol, 1 eq) in THE (400 mL) was added 2-[tert-butyl(dimethyl)silyl]oxyethanol (20.8 g, 118 mmol, 1.5 eq), TBAI (2.91 g, 7.88 mmol, 0.1 eq) and KOH (13.3 g, 236 mmol, 3 eq). The resulting mixture was stirred at 25° C. for 12 h. On completion, the mixture was concentrated. The residue was purified by column chromatography to give 2-[(4-bromo-2-methyl-pyrazol-3-yl)methoxy]ethoxy-tert-butyl-dimethyl-silane (13.19 g, 37.76 mmol, 47.94% yield) as a brown oil.

Step 3. To a solution of 2-[(4-bromo-2-methyl-pyrazol-3-yl)methoxy]ethoxy-tert-butyl-dimethyl-silane (13.1 g, 37.5 mmol, 1 eq) in THE (132 mL) was added TBAF·3H₂O (17.8 g, 56.2 mmol, 1.5 eq). The resulting mixture was stirred at 25° C. for 12 h. On completion, the mixture was concentrated to give a residue. The residue was purified by column chromatography to give 2-[(4-bromo-2-methyl-pyrazol-3-yl)methoxy]ethanol (8.8 g, 37.43 mmol, 99.83% yield) as a colorless oil.

Step 4. The mixture of 2-[(4-bromo-2-methyl-pyrazol-3-yl)methoxy]ethanol (2.00 g, 8.51 mmol, 2 eq), 2-vinylphenol (511 mg, 4.25 mmol, 1 eq) and PPh₃ (2.45 g, 9.36 mmol, 2.2 eq) in 2-MeTHF (48 mL) was stirred at 25° C. for 30 min, then DIAD (1.89 g, 9.36 mmol, 2.2 eq) was added dropwise to the mixture at 0° C., the resulting mixture was stirred for another 24 h at 25° C. On completion, the mixture was concentrated to give a residue. The residue was purified by column chromatography (SiO₂, Petroleum ether/Ethyl acetate=10:1 to 3:1) to give 4-bromo-1-methyl-5-[2-(2-vinylphenoxy)ethoxymethyl]pyrazole (590 mg, 1.75 mmol, 41.13% yield) as a colorless oil. ¹H NMR (400 MHz, DMSO-d₆) δ=7.56-7.46 (m, 2H), 7.28-7.19 (m, 1H), 7.02-6.87 (m, 3H), 5.79 (dd, J=1.6, 17.9 Hz, 1H), 5.24 (dd, J=1.6, 11.2 Hz, 1H), 4.62 (s, 2H), 4.18-4.09 (m, 2H), 3.84 (s, 3H), 3.81-3.77 (m, 2H).

### General Method A: Preparation of (11E)-1-methyl-1,18,19,21-tetrahydro-8H-10,7,4-(ethan[1]yl[1,2]diylidene)pyrazolo[3,4-f][1,4,12,13]benzodioxadiazacyclooctadecine

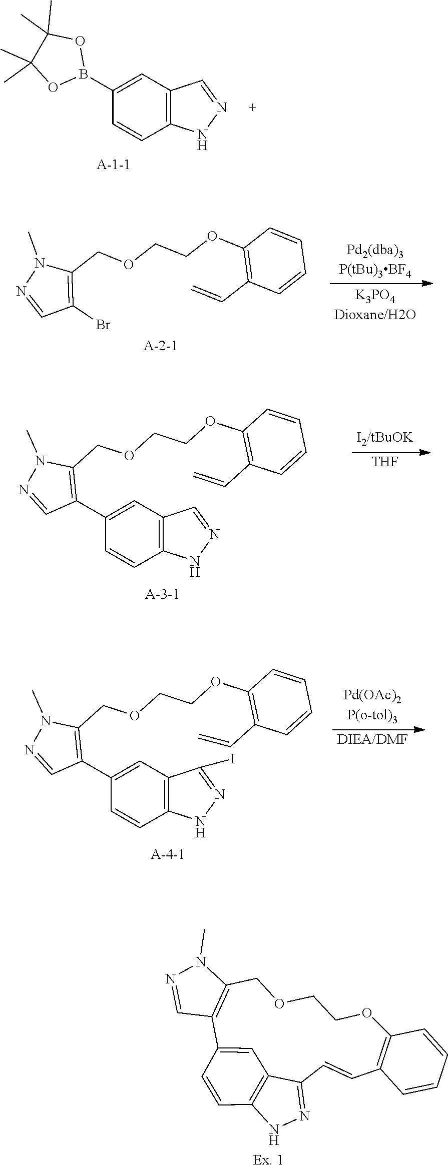

Step 1. To a mixture of 4-bromo-1-methyl-5-[2-(2-vinylphenoxy)ethoxymethyl]pyrazole (567 mg, 1.68 mmol, 1 eq) and 5-(4,4,5,5-tetramethyl-1,3,2-dioxaborolan-2-yl)-1H-indazole (492 mg, 2.02 mmol, 1.2 eq) in dioxane (12 mL)/H₂O (4 mL) was added K₃PO₄ (1.07 g, 5.04 mmol, 3 eq), tritert-butylphosphonium;tetrafluoroborate (48.8 mg, 0.168 mmol, 0.1 eq) and Pd₂(dba)₃ (77.0 mg, 84.1 mmol, 0.05 eq). The resulting mixture was stirred at 120° C. under N₂ for 24 h. On completion, the mixture was concentrated to give a residue. The residue was purified by column chromatography give 5-[1-methyl-5-[2-(2-vinylphenoxy) ethoxymethyl]pyrazol-4-yl]-1H-indazole (380 mg, 1.01 mmol, 60.36% yield) as a colorless oil. LCMS: m/z 375.1 (M+1).

Step 2. To a solution of 5-[1-methyl-5-[2-(2-vinylphenoxy)ethoxymethyl]pyrazol-4-yl]-1H-indazole (360 mg, 0.961 mmol, 1 eq) in THF (3.6 mL) was added tBuOK (324 mg, 2.88 mmol, 3 eq). The resulting mixture was stirred at 0° C. for 5 min, then I₂ (317 mg, 1.25 mmol, 1.3 eq) in THE (1 mL) was added dropwise. The resulting mixture was stirred at 25° C. for another 2 h. On completion, the mixture was filtered and concentrated. The residue was purified by column chromatography to give 3-iodo-5-[1-methyl-5-[2-(2-vinylphenoxy)ethoxymethyl]pyrazol-4-yl]-1H-indazole (345 mg, 0.690 mmol, 71.7% yield) as a yellow solid. LCMS: m/z 501.0 (M+1).

Step 3. To a solution of 3-iodo-5-[1-methyl-5-[2-(2-vinylphenoxy)ethoxymethyl]pyrazol-4-yl]-1H-indazole (100 mg, 0.200 mmol, 1 eq) in DMF (10 mL) was added tris-o-tolylphosphane (6.08 mg, 0.020 mmol, 0.1 eq), DIPEA (51.7 mg, 0.400 mmol, 2 eq) and Pd(OAc)₂ (2.24 mg, 0.01 mmol, 0.05 eq). The resulting mixture was stirred at 120° C. for 12 h. On completion, the mixture was filtered and concentrated to give a residue. The residue was purified by prep-HPLC to give Ex. 1 (20.42 mg, 0.0548 mmol, 27.43% yield) as an off-white solid.

### Preparation of 5-bromo-1-tetrahydropyran-2-yl-3-vinyl-indazole (B-1-1)

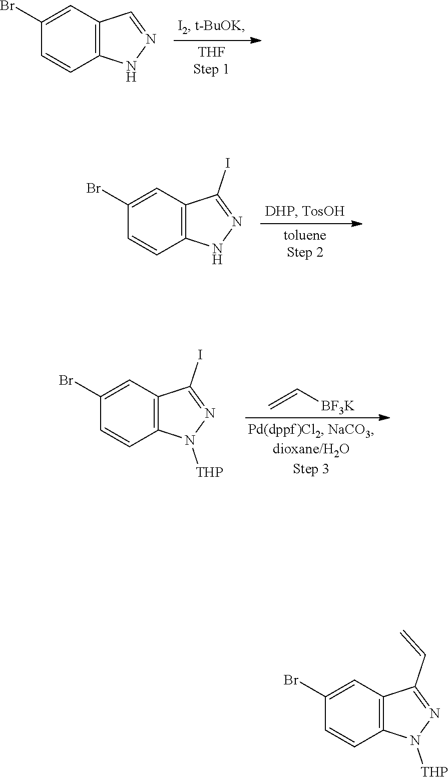

Step 1. To a solution of 5-bromo-1H-indazole (5.00 g, 25.3 mmol, 1 eq) in THF (50 mL) was added t-BuOK (5.70 g, 50.7 mmol, 2 eq) at 0° C. followed by addition of a solution of I₂ (7.08 g, 27.9 mmol, 1.1 eq) in THE (50 mL). The reaction mixture was stirred at 25° C. for 12 hrs. On completion, the residue was diluted with water and then extracted with EtOAc. The combined organic layer was dried over Na₂SO₄, filtered and concentrated in vacuo to give 5-bromo-3-iodo-1H-indazole (8.10 g, 98.8% yield) as brown solid. ¹H NMR (400 MHz, DMSO-d₆) δ=13.69 (s, 1H), 7.60 (d, J=1.2 Hz, 1H), 7.55-7.53 (m, 2H). LCMS: (M+1: 324.4)

Step 2. To a mixture of 5-bromo-3-iodo-1H-indazole (8.10 g, 25.0 mmol, 1 eq) and TosOH (863 mg, 5.02 mmol, 0.2 eq) in toluene (200 mL) was added DHP (5.27 g, 62.7 mmol, 2.5 eq). The reaction mixture was stirred at 90° C. for 12 hr. On completion, the residue was diluted with water and extracted with EtOAc. The combined organic layer was dried over Na₂SO₄, filtered, and concentrated in vacuo. The residue was purified by silica gel column chromatography to give 5-bromo-3-iodo-1-tetrahydropyran-2-yl-indazole (8.30 g, 81.2% yield) as yellow solid. ¹H NMR (400 MHz, DMSO-d₆) δ=7.75-7.70 (m, 1H), 7.62-7.58 (m, 2H), 5.83 (dd, J=2.0, 9.6 Hz, 1H), 3.89-3.82 (m, 1H), 3.75-3.67 (m, 1H), 2.35-2.29 (m, 1H), 2.01-1.93 (m, 2H), 1.74-1.67 (m, 1H), 1.59-1.53 (m, 2H). LCMS: (M+1: 408.7)

Step 3. To a mixture of 5-bromo-3-iodo-1-tetrahydropyran-2-yl-indazole (8.30 g, 20.3 mmol, 1 eq) and potassium hydride;trifluoro(vinyl)boron (2.73 g, 20.3 mmol, 1 eq) in dioxane (80 mL) and H₂O (12 ml) were added Pd(dppf)Cl₂ (1.49 g, 2.04 mmol, 0.1 eq) and Na₂CO₃ (6.48 g, 61.1 mmol, 3 eq). The reaction mixture was stirred at 80° C. for 12 hrs under N₂. On completion, the residue was diluted with water and extracted with EtOAc. The combined organic layer was dried over Na₂SO₄, filtered and concentrated in vacuo. The residue was purified by silica gel column chromatography to give 5-bromo-1-tetrahydropyran-2-yl-3-vinyl-indazole (6.00 g, 19.5 mmol, 95.7% yield) as white solid. ¹H NMR (400 MHz, DMSO-d₆) δ=8.24 (s, 1H), 7.73 (d, J=8.8 Hz, 1H), 7.57 (d, J=8.8 Hz, 1H), 7.01 (dd, J=11.6, 17.6 Hz, 1H), 6.14 (d, J=18.0 Hz, 1H), 5.85 (d, J=9.6 Hz, 1H), 5.53 (d, J=11.6 Hz, 1H), 3.88 (d, J=11.6 Hz, 1H), 3.78-3.69 (m, 1H), 2.43-2.31 (m, 1H), 2.06-1.91 (m, 2H), 1.79-1.66 (m, 1H), 1.58 (s, 2H).

### Preparation of tert-butyl-dimethyl-[2-[[2-methyl-4-(4,4,5,5-tetramethyl-1,3,2-dioxaborolan-2-yl)pyrazol-3-yl]methoxy]ethoxy]silane (B-2-1)

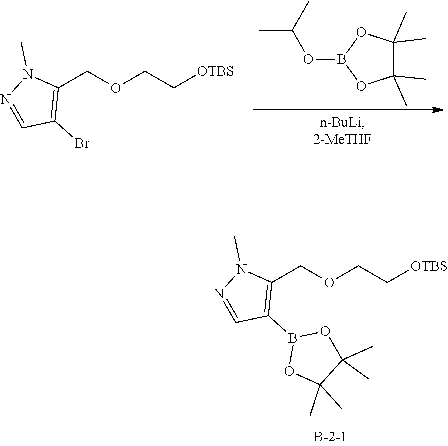 [SMILES: Cn1ncc(Br)c1COCCO[Si](C)(C)C(C)(C)C ; CC(C)OB1OC(C)(C)C(C)(C)O1 ; Cn1ncc(B2OC(C)(C)C(C)(C)O2)c1COCCO[Si](C)(C)C(C)(C)C]

To a solution of 2-[(4-bromo-2-methyl-pyrazol-3-yl)methoxy]ethoxy-tert-butyl-dimethyl-silane (2.0 g, 5.73 mmol, 1 eq) in 2-MeTHF (50 ml) was added n-BuLi (2.5 M, 3.44 mL, 1.5 eq) at −70° C. The mixture was stirred at −70° C. for 0.5 hr. Then 2-isopropoxy-4,4,5,5-tetramethyl-1,3,2-dioxaborolane (2.1 g, 11.4 mmol, 2 eq) was added and stirred at this temperature for 1.5 hrs. On completion, the mixture was added into NH₄Cl solution (60 mL), extracted with EA (3×100 mL), washed with brine (3×50 mL). The organic layer was dried over sodium sulfate, filtered and concentrated in vacuo to give a residue. The residue was purified by column chromatography (SiO₂, PE/EA=100/8) to give tert-butyl-dimethyl-[2-[[2-methyl-4-(4,4,5,5-tetramethyl-1,3,2-dioxaborolan-2-yl)pyrazol-3-yl]methoxy]ethoxy]silane (1.54 g, 2.02 mmol, 35.29% yield, 52% purity) was obtained as a yellow oil. ¹H NMR (400 MHz, CDCl₃) δ=7.69 (s, 1H), 4.58 (s, 2H), 3.92 (s, 3H), 3.79-3.77 (m, 2H), 3.75-3.72 (m, 2H), 1.31 (s, 12H), 0.89 (s, 9H), 0.07 (s, 6H).

### Preparation of tert-butyl-dimethyl-[3-[2-methyl-4-(4,4,5,5-tetramethyl-1,3,2-dioxaborolan-2-yl)pyrazol-3-yl]oxypropoxy]silane (B-2-2)

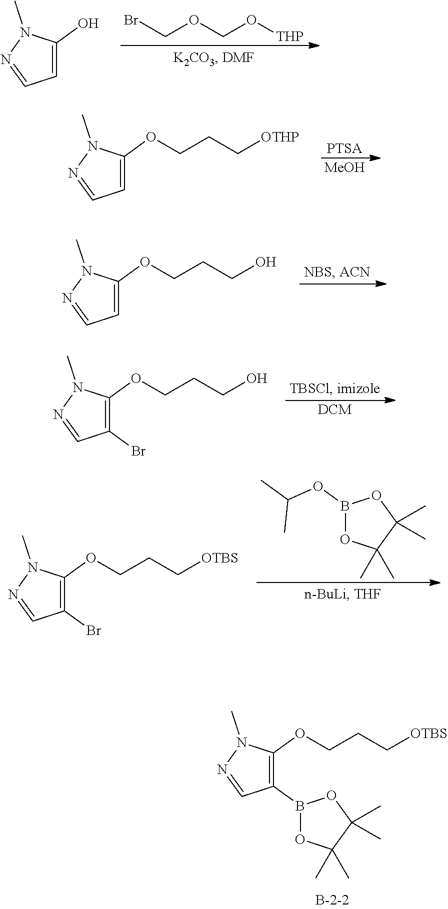

Step 1. To a mixture of 2-methylpyrazol-3-ol (16.5 g, 168 mmol, 1 eq) and K₂CO₃ (69.5 g, 503 mmol, 3.00 eq) in DMF (700 mL) was added 2-(3-bromopropoxy)tetrahydropyran (56.1 g, 251 mmol, 1.5 eq). The resulting mixture was stirred at 40° C. for 12 hours. On completion, the mixture was added water (3 L) and extracted with ethyl acetate (500 ml * 5). The combined organic phase was dried over anhydrous sodium sulfate, filtered and concentrated. The residue was purified by column chromatography to give 1-methyl-5-(3-tetrahydropyran-2-yloxypropoxy)pyrazole (27.0 g, 112 mmol, 66.97% yield) as yellow oil. ¹H NMR (400 MHz, CDCl₃) δ=7.25 (d, J=2.0 Hz, 1H), 5.46 (d, J=2.0 Hz, 1H), 4.59-4.54 (m, 1H), 4.15-4.09 (m, 2H), 3.88 (td, J=6.4, 10.0 Hz, 1H), 3.80 (ddd, J=3.2, 8.0, 11.2 Hz, 1H), 3.60 (s, 3H), 3.56-3.47 (m, 2H), 2.05 (t, J=6.4 Hz, 2H), 1.83-1.74 (m, 1H), 1.72-1.65 (m, 1H), 1.57-1.47 (m, 4H).

Step 2. A mixture of 1-methyl-5-(3-tetrahydropyran-2-yloxypropoxy)pyrazole (27.0 g, 112 mmol, 1 eq), PTSA (3.87 g, 22.4 mmol, 0.2 eq) in MeOH (40 mL) was stirred at 60° C. for 16 hours. On completion, the mixture was concentrated in vacuum. It was added NaHCO₃ solution to adjust pH to 7 and extracted with ethyl acetate (100 mL*4). The combined organic phase was dried over anhydrous sodium sulfate, filtered and concentrated to give 3-(2-methylpyrazol-3-yl)oxypropan-1-ol (11.9 g, 76.1 mmol, 67.81% yield) as yellow oil. ¹H NMR (400 MHz, DMSO-d₆) δ=7.19 (d, J=2.0 Hz, 1H), 5.61 (d, J=2.0 Hz, 1H), 4.58 (t, J=5.2 Hz, 1H), 4.10 (t, J=6.4 Hz, 2H), 3.58-3.53 (m, 2H), 3.52 (s, 3H), 1.86 (quin, J=6.4 Hz, 2H).

Step 3. A mixture of 3-(2-methylpyrazol-3-yl)oxypropan-1-ol (11.7 g, 74.9 mmol, 1 eq) in MeCN (250 mL) was added NBS (13.7 g, 77.1 mmol, 1.03 eq). The mixture was stirred at 25° C. for 1.5 hours. On completion, the mixture was concentrated in vacuum to give crude which was purified by prep-HPLC to give 3-(4-bromo-2-methyl-pyrazol-3-yl)oxypropan-1-ol (10.1 g, 42.9 mmol, 57.35% yield) as yellow oil. ¹H NMR (400 MHz, CDCl₃) δ=7.28 (s, 1H), 4.40 (t, J=6.0 Hz, 2H), 3.86 (t, J=6.0 Hz, 2H), 3.67 (s, 3H), 2.14 (s, 1H), 2.03 (quin, J=6.0 Hz, 2H).

Step 4. To a solution of 3-(4-bromo-2-methyl-pyrazol-3-yl)oxypropan-1-ol (1.00 g, 4.25 mmol, 1.0 eq) in DCM (20 mL) was added imidazole (579 mg, 8.51 mmol, 2.0 eq). Then tert-butyl-chloro-dimethyl-silane (962 mg, 6.38 mmol, 782 p L, 1.5 eq) was added at 0° C. The reaction mixture was stirred at 25° C. for 1 hr. The mixture was added to water (30 mL), extracted with DCM (3×30 mL). The combined organic layers were washed with brine (2×20 mL), dried over Na₂SO₄, filtered and concentrated in vacuum to give 3-(4-bromo-2-methyl-pyrazol-3-yl)oxypropoxy-tert-butyl-dimethyl-silane (1.40 g, 3.89 mmol, 91.4% yield) as a yellow oil. ¹H NMR (400 MHz, DMSO-d₆) δ=7.38 (s, 1H), 4.28 (t, J=6.0 Hz, 2H), 3.77-3.73 (m, 2H), 3.62 (s, 3H), 1.89 (t, J=6.0 Hz, 2H), 0.86 (s, 9H), 0.04 (s, 6H).

Step 5. To a mixture of 3-(4-bromo-2-methyl-pyrazol-3-yl)oxypropoxy-tert-butyl-dimethyl-silane (1.30 g, 3.72 mmol, 1.0 eq) in 2-MeTHF (20 ml) was added n-BuLi (1 M, 7.44 mL, 2.0 eq) at −78° C. for 0.5 hr, then 2-isopropoxy-4,4,5,5-tetramethyl-1,3,2-dioxaborolane (2.08 g, 11.2 mmol, 2.28 mL, 3.0 eq) was added at −78° C. for 1 hr. The mixture was diluted with water (40 mL), extracted with EA (3×30 mL). The combined organic layer was washed with brine (30 mL), dried over Na₂SO₄, filtered and concentrated in vacuum to give tert-butyl-dimethyl-[3-[2-methyl-4-(4,4,5,5-tetramethyl-1,3,2-dioxaborolan-2-yl)pyrazol-3-yl]oxypropoxy]silane (1.40 g, 1.17 mmol, 31.3% yield, 33.0% purity) as a yellow oil.

### Preparation of tert-butyl-dimethyl-[1-methyl-3-[2-methyl-4-(4,4,5,5-tetramethyl-1,3,2-dioxaborolan-2-yl)pyrazol-3-yl]oxy-propoxy]silane (B-2-3)

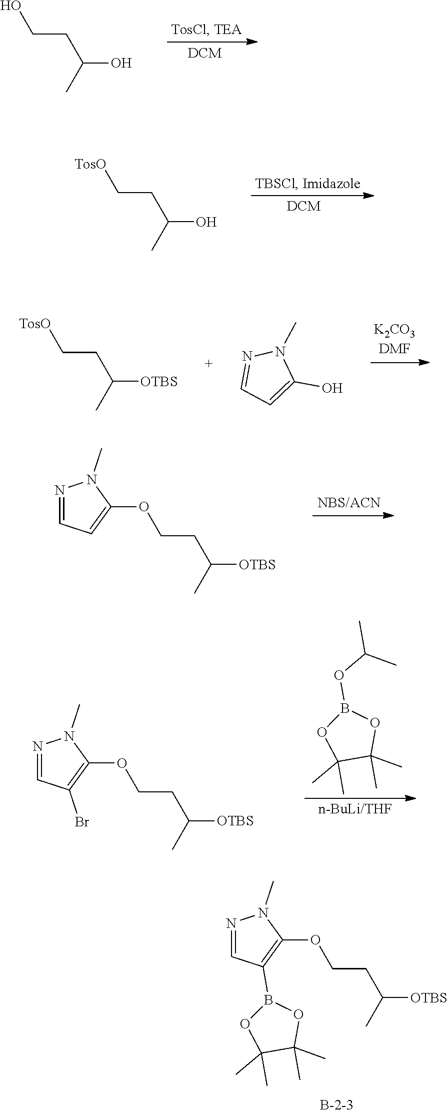

Step 1. To a solution of butane-1,3-diol (5.35 g, 59.4 mmol, 1.0 eq) in DCM (50 mL) were added TEA (18.0 g, 178 mmol, 24.8 mL, 3.0 eq) and DMAP (218 mg, 1.78 mmol, 0.03 eq) followed by addition of 4-methylbenzenesulfonyl chloride (12.5 g, 65.3 mmol, 1.1 eq) at 0° C. The mixture was stirred at 0-20° C. for 16 hr, quenched with water (200 mL), and extracted with ethyl acetate (100 mL×3). The combined organic phase was dried over anhydrous sodium sulfate, filtered and concentrated. The residue was purified by column chromatography to give 3-hydroxybutyl 4-methylbenzenesulfonate (15.2 g, crude) as a colorless oil. ¹H NMR (400 MHz, CDCl₃) δ=7.73 (s, 1H), 7.71 (s, 1H), 7.29 (s, 1H), 7.27 (s, 1H), 4.16 (dd, J=5.2, 10.0 Hz, 1H), 4.07-4.01 (m, 2H), 3.90-3.81 (m, 1H), 3.90-3.80 (m, 1H), 2.38 (s, 4H), 1.96-1.93 (m, 1H), 1.79-1.70 (m, 1H), 1.67-1.57 (m, 1H), 1.10 (d, J=6.0 Hz, 3H).

Step 2. To a solution of 3-hydroxybutyl 4-methylbenzenesulfonate (15.0 g, 61.4 mmol, 1.0 eq) in DCM (150 mL) and imidazole (8.36 g, 123 mmol, 2.0 eq) was added TBSCl (11.1 g, 73.7 mmol, 9.03 mL, 1.2 eq) at 0° C. The mixture was stirred at 25° C. for 1 hr. The mixture was quenched with water (500 mL) and extracted with ethyl acetate (250 mL×3), the combined organic phase was dried over anhydrous sodium sulfate, filtered and concentrated to give 3-[tert-butyl(dimethyl)silyl]oxybutyl 4-methylbenzenesulfonate (23.4 g, crude) as a colorless oil. ¹H NMR (400 MHz, DMSO-d₆) δ=7.86 (s, 1H), 7.84 (s, 1H), 7.57 (s, 1H), 7.55 (s, 1H), 4.16-4.09 (m, 2H), 3.92 (ddd, J=4.2, 6.0, 7.6 Hz, 1H), 2.59 (s, 3H), 1.83-1.65 (m, 2H), 1.12 (d, J=6.0 Hz, 3H), 0.86 (s, 9H), 0.07 (s, 3H), 0.03 (s, 3H).

Step 3. To a solution of 2-methylpyrazol-3-ol (5.20 g, 53.0 mmol, 1.0 eq) in DMF (50 mL) were added K₂CO₃ (22.0 g, 159 mmol, 3.0 eq) and 3-[tert-butyl(dimethyl)silyl]oxybutyl 4-methylbenzenesulfonate (22.8 g, 63.6 mmol, 1.2 eq). The mixture was stirred at 80° C. for 16 hr, quenched with water (200 mL) and extracted with ethyl acetate (50 mL×3). The combined organic phase was dried over anhydrous sodium sulfate, filtered and concentrated. The residue was purified by column chromatography to give tert-butyl-dimethyl-[1-methyl-3-(2-methylpyrazol-3-yl)oxy-propoxy]silane (8.2 g, 28.8 mmol, 54.4% yield) as a colorless oil. ¹H NMR (400 MHz, DMSO-d₆) δ=7.18 (d, J=2.0 Hz, 1H), 5.57 (d, J=2.0 Hz, 1H), 4.13-3.99 (m, 3H), 3.52 (s, 3H), 1.92-1.82 (m, 1H), 1.73 (tdd, J=5.2, 8.4, 13.8 Hz, 1H), 1.15 (d, J=6.0 Hz, 3H), 0.84 (s, 9H), 0.04 (s, 3H), −0.01 (s, 3H).

Step 4. To a solution of tert-butyl-dimethyl-[1-methyl-3-(2-methylpyrazol-3-yl) oxy-propoxy]silane (8.00 g, 28.12 mmol, 1.0 eq) in ACN (80 ml) was added NBS (5.26 g, 29.5 mmol, 1.05 eq) at 0° C. The mixture was stirred at 0-25° C. for 2 h, and then quenched with sat. aqueous Na₂SO₃(200 mL) and extracted with ethyl acetate (100 mL×3). The combined organic phase was dried over anhydrous sodium sulfate, filtered and concentrated to give a residue. The residue was purified by column chromatography to give [3-(4-bromo-2-methyl-pyrazol-3-yl)oxy-1-methyl-propoxy]-tert-butyl-dimethyl-silane (5.1 g, 13.70 mmol, 48.7% yield) as a red oil. LCMS: m/z 364.9 (M+1).

Step 5. To a solution of [3-(4-bromo-2-methyl-pyrazol-3-yl)oxy-1-methyl-propoxy]-tert-butyl-dimethyl-silane (5.00 g, 13.8 mmol, 1.0 eq) in THF (50 mL) was added n-BuLi (2.5 M, 13.8 mL, 2.5 eq) at −70° C. The mixture was stirred at −70° C. for 1 h. Then 2-isopropoxy-4,4,5-trimethyl-1,3,2-dioxaborolane (7.10 g, 41.3 mmol, 3.0 eq) was added. The mixture was stirred at −70° C. for 3 h, then quenched with NH₄Cl (200 mL) and extracted with ethyl acetate (100 mL×3). The combined organic phase was dried over anhydrous sodium sulfate, filtered and concentrated. The residue was purified by column chromatography to give tert-butyl-dimethyl-[1-methyl-3-[2-methyl-4-(4,4,5,5-tetramethyl-1,3,2-dioxaborolan-2-yl)pyrazol-3-yl]oxy-propoxy]silane (3.25 g, 5.70 mmol, 41.4% yield) as a colorless oil. LCMS: m/z 411.3 (M+1).

### Preparation of tert-butyl-dimethyl-[4-[2-methyl-4-(4,4,5,5-tetramethyl-1,3,2-dioxaborolan-2-yl)pyrazol-3-yl]oxybutoxy]silane (B-2-4)

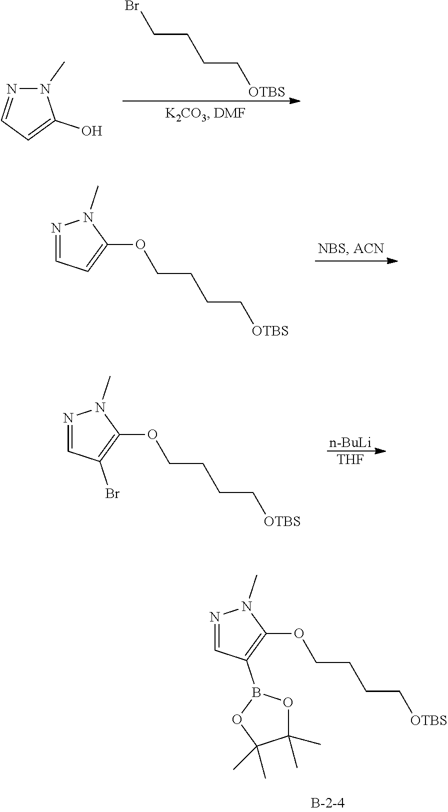

Step 1. To a solution of 2-methylpyrazol-3-ol (2.50 g, 25.5 mmol, 1 eq) and 4-bromobutoxy-tert-butyl-dimethyl-silane (8.17 g, 30.6 mmol, 1.2 eq) in DMF (50 mL) was added K₂CO₃ (10.6 g, 76.5 mmol, 3 eq). The resulting mixture was stirred at 80° C. for 2 hr.

On completion, the mixture was extracted with ethyl acetate (25 mL×3). The combined organic phase was dried over anhydrous sodium sulfate, filtered and concentrated. The residue was purified by column chromatography to give 5-(4-((tert-butyldimethylsilyl)oxy)butoxy)-1-methyl-1H-pyrazole (5.30 g, 18.5 mmol, 65.0% yield) as a yellow oil. ¹H NMR (400 MHz, DMSO-d₆) δ=7.17 (s, 1H), 5.58 (s, 1H), 4.04 (t, J=6.8 Hz, 2H), 3.66-3.66 (m, 1H), 3.67-3.60 (m, 1H), 3.53-3.49 (m, 3H), 1.80-1.72 (m, 2H), 1.64-1.55 (m, 2H), 1.40-1.32 (m, 1H), 0.86 (s, 10H), 0.05-0.02 (m, 6H).

Step 2. To a solution of tert-butyl-dimethyl-[4-(2-methylpyrazol-3-yl)oxybutoxy]silane (5.10 g, 17.9 mmol, 1 eq) in ACN (50 mL) was added dropwise NBS (3.51 g, 19.7 mmol, 1.1 eq) at 0° C. On completion, the mixture was quenched with sat. aqueous Na₂SO₃ (20 mL) and extracted with ethyl acetate (25 mL×3). The combined organic phase was dried over anhydrous sodium sulfate, filtered and concentrated. The residue was purified by column chromatography to give 4-bromo-5-(4-((tert-butyldimethylsilyl)oxy)butoxy)-1-methyl-1H-pyrazole (3.00 g, 8.22 mmol, 44.2% yield) as a yellow oil. 1H NMR (400 MHz, DMSO-d6) δ=7.40 (s, 1H), 4.24-4.19 (m, 2H), 3.66-3.62 (m, 2H), 3.62 (s, 3H), 1.86-1.69 (m, 2H), 1.69-1.54 (m, 2H), 0.88-0.82 (m, 9H), 0.07-0.02 (m, 6H)

Step 3. 4-Bromo-5-(4-((tert-butyldimethylsilyl)oxy)butoxy)-1-methyl-1H-pyrazole was converted to B-2-4 using the same condition as Step 5 in B-2-2.

### Preparation of tert-butyl-dimethyl-[2-methyl-5-[2-methyl-4-(4,4,5,5-tetramethyl-1,3,2-dioxaborolan-2-yl)pyrazol-3-yl]oxy-pentoxy]silane (B-2-5)

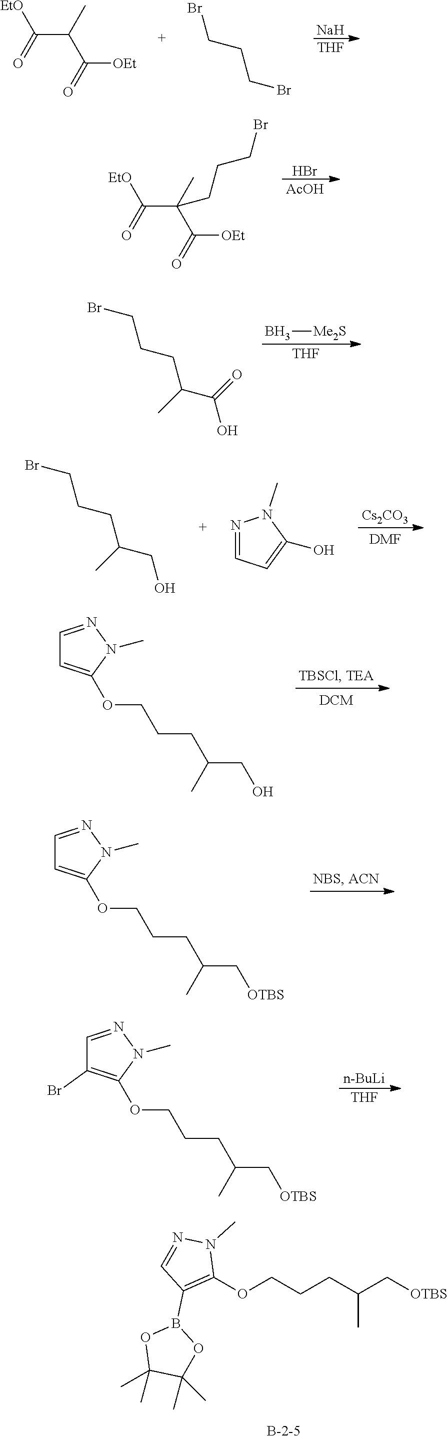

Step 1. A mixture of diethyl 2-methylpropanedioate (20.0 g, 115 mmol, 19.6 mL, 1 eq) in THF (300 mL) the mixture was stirred at 0° C. followed by addition of NaH (11.5 g, 287 mmol, 60% purity, 2.5 eq). Then the mixture was stirred at 25° C. for 0.5 hr and 1,3-dibromopropane (69.5 g, 344 mmol, 35.1 mL, 3 eq) was added to the reaction mixture. And then the mixture was stirred at 25° C. for 1 h and at 60° C. for 16 h. On completion, the mixture was quenched by H₂O (250 mL) and extracted with EtOAc (150 mL*3). The combined organic layer was dried over anhydrous Na₂SO₄, filtered and the filtrate was concentrated in vacuum. The residue was purified by column chromatography to give diethyl 2-(3-bromopropyl)-2-methyl-propanedioate (18.8 g, 63.7 mmol, 55% yield) as a colorless oil. ¹H NMR (400 MHz, CDCl₃) δ=4.22-4.14 (m, 4H), 3.39 (t, J=6.4 Hz, 2H), 2.01-1.95 (m, 2H), 1.89-1.79 (m, 2H), 1.41 (s, 3H), 1.25 (t, J=7.2 Hz, 6H).

Step 2. A solution of diethyl 2-(3-bromopropyl)-2-methyl-propanedioate (18.6 g, 63.0 mmol, 1 eq) and HBr in HOAc (93 mL, 48% purity) was heated to reflux for 16 h in a 100° C. On completion, the mixture was quenched by H₂O (200 mL) and extracted with EtOAc (100 mL*3). The combined organic layer was dried over anhydrous Na₂SO₄, filtered and the filtrate was concentrated in vacuum to give 5-bromo-2-methyl-pentanoic acid (9.10 g, 46.7 mmol, 74% yield) as a brown oil, which was used into the next step without further purification. ¹H NMR (400 MHz, CDCl₃) δ=3.46-3.37 (m, 2H), 2.54-2.46 (m, 1H), 1.97-1.84 (m, 3H), 1.69-1.60 (m, 1H), 1.24-1.21 (m, 3H).

Step 3. To a solution of 5-bromo-2-methyl-pentanoic acid (9.00 g, 46.1 mmol, 1 eq) in THE (90 mL) at 0° C. was added BH₃-Me₂S (10 M, 23.1 mL, 5 eq). The mixture was stirred at 20° C. for 3 h. On completion, the reaction mixture was quenched with MeOH (30 mL). The mixture was filtered and concentrated under reduced pressure to give 5-bromo-2-methyl-pentan-1-ol (9.37 g, crude) as a brown oil, which was used in the next step without further purification. ¹H NMR (400 MHz, CDCl₃) δ=3.53-3.45 (m, 2H), 3.44-3.37 (m, 2H), 1.96-1.83 (m, 2H), 1.68-1.63 (m, 2H), 1.20-1.15 (m, 1H), 0.94 (d, J=6.8 Hz, 3H).

Step 4. A mixture of 5-bromo-2-methyl-pentan-1-ol (9.37 g, 51.8 mmol, 1 eq), 2-methylpyrazol-3-ol (11.2 g, 114 mmol, 2.2 eq), Cs₂CO₃ (50.6 g, 155 mmol, 3 eq) in DMF (94 mL) was stirred at 80° C. for 16 h. On completion, the residue was diluted with H₂O (500 mL) and extracted with EtOAc (100 mL*3). The combined organic layer was dried over anhydrous Na₂SO₄, filtered and the filtrate was concentrated in vacuum. The residue was purified by column chromatography to give 2-methyl-5-(2-methylpyrazol-3-yl)oxy-pentan-1-ol (3.20 g, 16.1 mmol, 31% yield) as a brown oil. LCMS: m/z 199.0 (M+1).

Step 5. A mixture of 2-methyl-5-(2-methylpyrazol-3-yl)oxy-pentan-1-ol (3.20 g, 16.1 mmol, 1 eq), TBSCl (3.65 g, 24.2 mmol, 2.97 mL, 1.5 eq), imidazole (2.20 g, 32.3 mmol, 2 eq) in DCM (18 mL) was stirred at 0° C., and then at 25° C. for 16 h. On completion, the mixture was filtered and concentrated under reduced pressure. The residue was purified by column chromatography to give tert-butyl-dimethyl-[2-methyl-5-(2-methylpyrazol-3-yl)oxy-pentoxy]silane (4.4 g, 14.1 mmol, 87% yield) as a brown oil. LCMS: m/z 313.6 (M+1).

Step 6. To a solution of tert-butyl-dimethyl-[2-methyl-5-(2-methylpyrazol-3-yl)oxy-pentoxy]silane (4.40 g, 14.1 mmol, 1 eq) in ACN (44 mL) was added NBS (2.51 g, 14.1 mmol, 1 eq) at 0° C. The mixture was stirred at 0-25° C. for 3 h. On completion, the reaction mixture was quenched by addition of sat. aqueous Na₂SO₃ (25 mL) at 20° C., and extracted with EtOAc (30 mL*3). The combined organic layer was dried over anhydrous Na₂SO₄, filtered and the filtrate was concentrated in vacuum. The residue was purified by column chromatography to give {[5-(4-bromo-2-methyl-pyrazol-3-yl)oxy-2-methyl-pentoxy]-tert-butyl-dimethyl-silane (3.30 g, 8.43 mmol, 60% yield) as a brown oil. LCMS: m/z 392.9 (M+1)

Step 7. To a solution of [5-(4-bromo-2-methyl-pyrazol-3-yl)oxy-2-methyl-pentoxy]-tert-butyl-dimethyl-silane (4.60 g, 11.75 mmol, 1 eq) in THF (70 mL) at −78° C. was added n-BuLi (2.5 M, 9.40 mL, 2 eq) and stirred at −78° C. for 0.5 h followed by addition of 2-isopropoxy-4,4,5,5-tetramethyl-1,3,2-dioxaborolane (6.56 g, 35.3 mmol, 7.19 mL, 3 eq). The mixture was stirred at −78° C. for 1.5 h. On completion, the mixture was quenched by sat. aqueous NH₄Cl (50 mL) and extracted with EtOAc (50 mL*3). The combined organic layer was dried over anhydrous Na₂SO₄, filtered and the filtrate was concentrated in vacuum. The residue was purified by column chromatography to give tert-butyl-dimethyl-[2-methyl-5-[2-methyl-4-(4,4,5,5-tetramethyl-1,3,2-dioxaborolan-2-yl)pyrazol-3-yl]oxy-pentoxy]silane (4.75 g, 10.8 mmol, 92% yield) as a brown oil. LCMS: 439.2 (M+1).

### Preparation of tert-butyl N-[2-[tert-butyl(dimethyl) silyl]oxyethyl]-N-[[2-methyl-4-(4,4,5,5-tetramethyl-1,3,2-dioxaborolan-2-yl)pyrazol-3-yl]methyl]carbamate (B-2-6)

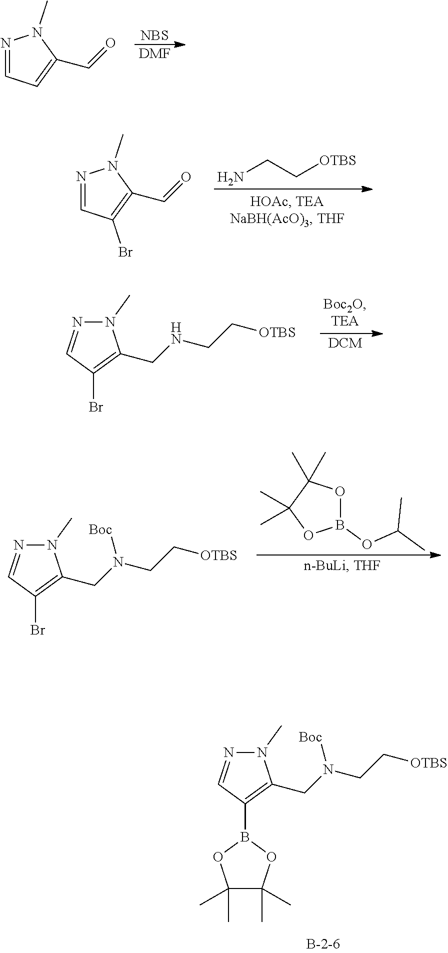

Step 1. To a mixture of 2-methylpyrazole-3-carbaldehyde (20.0 g, 181 mmol, 1 eq) in DMF (350 mL) was added NBS (32.3 g, 181 mmol, 1 eq) at 0° C. The reaction mixture was stirred at 25° C. for 12 hours. On completion, the reaction mixture was quenched with water (1000 mL), filtered and dried to afford 4-bromo-2-methyl-pyrazole-3-carbaldehyde (34.0 g, 179 mmol, 99% yield) as a white solid. ¹H NMR (400 MHz, DMSO-d₆) δ 9.85 (s, 1H), 7.78 (s, 1H), 4.09 (s, 3H).

Step 2. To a mixture of 2-[tert-butyl(dimethyl)silyl]oxyethanamine (13.2 g, 75.6 mmol, 1.1 eq) in THE (20 mL) was added TEA (6.96 g, 68.7 mmol, 9.57 mL, 1 eq) followed by addition of 4-bromo-2-methyl-pyrazole-3-carbaldehyde (13.0 g, 68.7 mmol, 1 eq) and HOAc (4.13 g, 68.7 mmol, 3.93 mL, 1 eq). The reaction mixture was stirred at 60° C. for 0.5 hour. To the reaction mixture was then added NaBH(OAc)₃ (18.9 g, 89.4 mmol, 1.3 eq). The reaction mixture was stirred at 60° C. for 12 hours. On completion, the mixture was diluted with water (90 mL) and extracted with EA (2×100 mL). The combined organic layers were dried over Na₂SO₄, filtered, and concentrated in vacuo. The residue was purified by column chromatography to afford N-[(4-bromo-2-methyl-pyrazol-3-yl)methyl]-2-[tert-butyl(dimethyl)silyl]oxy-ethanamine (6.00 g, 17.2 mmol, 25% yield) as yellow oil. ¹H NMR (400 MHz, DMSO-d₆) δ 7.42 (s, 1H), 3.83-3.82 (m, 3H), 3.75 (s, 2H), 3.58 (t, J=6.0 Hz, 2H), 3.29 (s, 2H), 1.97 (s, 1H), 0.83 (s, 9H), 0.01 (m, 6H).

Step 3. To a mixture of N-[(4-bromo-2-methyl-pyrazol-3-yl)methyl]-2-[tert-butyl(dimethyl) silyl]oxy-ethanamine (11.0 g, 31.5 mmol, 1 eq) in DCM (100 mL) was added TEA (9.59 g, 94.7 mmol, 13.1 mL, 3 eq) and Boc₂O (10.3 g, 47.3 mmol, 10.8 mL, 1.5 eq). The reaction mixture was stirred at 25° C. for 12 hours. On completion, the reaction mixture was concentrated in vacuo. The residue was purified by column chromatography to afford tert-butyl N-[(4-bromo-2-methyl-pyrazol-3-yl)methyl]-N-[2-[tert-butyl(dimethyl)silyl]oxyethyl]carbamate (9.40 g, 20.9 mmol, 66% yield) as yellow oil. ¹H NMR (400 MHz, DMSO-d₆) δ 7.50 (s, 1H), 4.56 (s, 2H), 3.78 (s, 3H), 3.59 (t, J=6.0 Hz, 2H), 3.20-3.10 (m, 2H), 1.44-1.36 (m, 9H), 0.86 (s, 9H), 0.02 (s, 6H)

Step 4. To a mixture of tert-butyl N-[(4-bromo-2-methyl-pyrazol-3-yl)methyl]-N-[2-[tert-butyl(dimethyl)silyl]oxyethyl]carbamate (7.50 g, 16.7 mmol, 1 eq) in THF (100 mL) was added n-BuLi (2.5 M, 20.0 mL, 3 eq) at −70° C. The reaction mixture was stirred at −70° C. for 0.5 hour followed by addition of 2-isopropoxy-4,4,5,5-tetramethyl-1,3,2-dioxaborolane (9.33 g, 50.1 mmol, 10.2 mL, 3 eq). The reaction mixture was stirred at −70° C. for 1.5 hours. On completion, the residue was diluted with water (80 mL) and extracted with EA (2×90 mL). The combined organic layers were dried over Na₂SO₄, filtered and concentrated in vacuo. The residue was purified by column chromatography to afford tert-butyl N-[2-[tert-butyl(dimethyl) silyl]oxyethyl]-N-[[2-methyl-4-(4,4,5,5-tetramethyl-1,3,2-dioxaborolan-2-yl)pyrazol-3-yl]methyl]carbamate (7.00 g, 14.1 mmol, 84% yield) as yellow oil. ¹H NMR (400 MHz, DMSO-d₆) δ 7.50 (s, 1H), 4.68 (s, 2H), 3.74 (s, 3H), 3.47 (s, 2H), 3.13 (s, 2H), 1.41 (s, 9H), 1.25 (s, 12H), 0.83 (s, 9H), −0.01 (s, 6H)

### Preparation of 5-(1-(2-((tert-butyldimethylsilyl)oxy)ethoxy)ethyl)-1-methyl-4-(4,4,5,5-tetramethyl-1,3,2-dioxaborolan-2-yl)-1H-pyrazole (B-2-7)

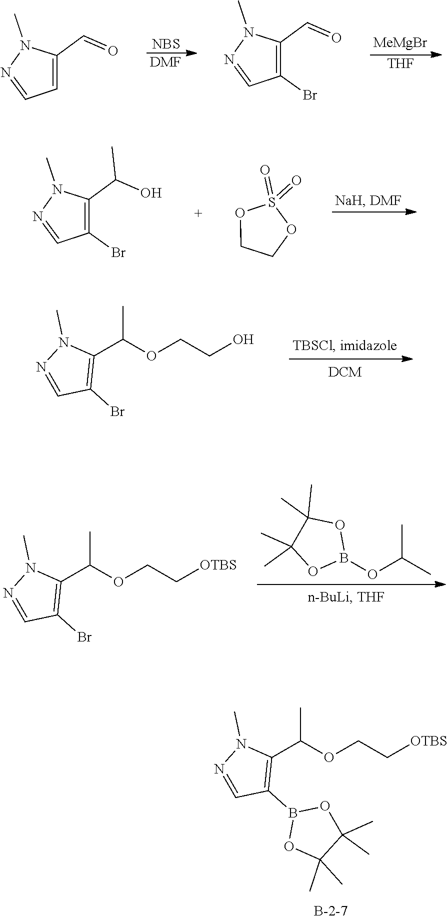

Step 1. To a solution of 2-methylpyrazole-3-carbaldehyde (25.0 g, 227 mmol, 1 eq) in DMF (250 mL) was added NBS (40.4 g, 227 mmol, 1 eq) at 0° C. The mixture was stirred at 25° C. for 16 h. To the mixture was added water (150 mL), and the mixture was extracted with EtOAc (50 mL*5). The combined organic layer was dried over anhydrous sodium sulfate, filtered and concentrated to give 4-bromo-1-methyl-1H-pyrazole-5-carbaldehyde (40.0 g, 222 mmol, 93.2% yield) as a white solid. LCMS: m/z 190.8 (M+1)

Step 2. To a solution of 4-bromo-2-methyl-pyrazole-3-carbaldehyde (36.0 g, 190 mmol, 1 eq) in THF (360 mL) was added MeMgBr (3 M, 95.23 mL, 1.5 eq) at 0° C. after degassing and purging with N₂ for 3 times. The mixture was stirred at 25° C. for 2 h under N₂ atmosphere. The reaction mixture was quenched by addition NH₄Cl 600 mL at 0° C., and then diluted with H₂O (100 mL) and extracted with EtOAc (100 mL*3). The combined organic layers were dried over Na₂SO₄, filtered and concentrated under reduced pressure. The residue was purified by column chromatography to give 1-(4-bromo-1-methyl-1H-pyrazol-5-yl)ethanol (27.0 g, 132 mmol, 69.1% yield) as a yellow oil. LCMS: m/z 206.9 (M+1).

Step 3. To a solution of 1-(4-bromo-2-methyl-pyrazol-3-yl)ethanol (27.0 g, 131 mmol, 1 eq) in DMF (270 mL) was added NaH (10.5 g, 263 mmol, 60% purity, 2 eq) at 0° C. The mixture was stirred at 0° C. for 0.5 h. Then 1,3,2-dioxathiolane 2,2-dioxide (32.7 g, 263 mmol, 2 eq) was added. The mixture was stirred at 25° C. for 16 hr. The mixture was quenched with aqueous HCl (1 M, 350 mL), and the mixture was extracted with EtOAc. The combined organic phase was dried over anhydrous sodium sulfate, filtered and concentrated. The residue was purified by column chromatography to give 2-(1-(4-bromo-1-methyl-1H-pyrazol-5-yl)ethoxy)ethanol (40.0 g, 160 mmol, crude) as a yellow oil. ¹H NMR (400 MHz, DMSO-d₆) δ=8.15 (s, 3H), 7.99-7.90 (m, 8H), 7.48-7.44 (m, 1H), 4.84-4.68 (m, 1H), 4.26-4.03 (m, 2H), 3.87 (d, J=5.8 Hz, 3H), 3.62-3.43 (m, 3H), 3.41-3.22 (m, 2H), 2.88 (s, 26H), 2.49 (t, J=5.2 Hz, 19H), 1.44-1.36 (m, 3H).

Step 4. To a solution of 2-[1-(4-bromo-2-methyl-pyrazol-3-yl)ethoxy]ethanol (37.0 g, 148 mmol, 1 eq) in DCM (370 mL) was added imidazole (20.2 g, 297 mmol, 2 eq), TBSCl (33.6 g, 223 mmol, 1.5 eq) at 0° C. The mixture was stirred at 25° C. for 2 h and extracted with dichloromethane (60 mL×3), the combined organic phase was dried over anhydrous sodium sulfate, filtered and concentrated. The residue was purified by column chromatography to give 4-bromo-5-(1-(2-((tert-butyldimethylsilyl)oxy)ethoxy)ethyl)-1-methyl-1H-pyrazole (11.0 g, 30.3 mmol, 20.4% yield) as a colorless oil. LCMS: m/z 364.9 (M+1).

Step 5. A mixture of 2-[1-(4-bromo-2-methyl-pyrazol-3-yl)ethoxy]ethoxy-tert-butyl-dimethyl-silane (5.8 g, 15.9 mmol, 1 eq) and 2-isopropoxy-4,4,5,5-tetramethyl-1,3,2-dioxaborolane (8.91 g, 47.89 mmol, 3 eq) in THE (58 mL) was degassed and purged with N₂ for 3 times followed by addition of n-BuLi (2.5 M, 19.15 mL, 3 eq). The mixture was stirred at −68° C. for 2 h under N₂ atmosphere and then warmed to ambient temperature. The mixture was quenched with sat. aqueous NH₄Cl (20 mL) and extracted with ethyl acetate (25 mL×3), the combined organic phase was dried over anhydrous sodium sulfate, filtered and concentrated to give a residue. The residue was purified by column chromatography to give 5-(1-(2-((tert-butyldimethylsilyl)oxy)ethoxy)ethyl)-1-methyl-4-(4,4,5,5-tetramethyl-1,3,2-dioxaborolan-2-yl)-1H-pyrazole (5.20 g, 12.4 mmol, 77.8% yield) as a colorless solid. ¹H NMR (400 MHz, DMSO-d₆) δ=7.45 (s, 1H), 5.18-5.11 (m, 1H), 3.92-3.84 (m, 3H), 3.70-3.59 (m, 2H), 3.34-3.26 (m, 1H), 3.24-3.17 (m, 1H), 1.43-1.39 (m, 3H), 1.24 (s, 12H), 0.85-0.80 (m, 9H), 0.00 (s, 6H).

### Preparation of tert-butyl-dimethyl-[3-[[4-(4,4,5,5-tetramethyl-1,3,2-dioxaborolan-2-yl)-1H-pyrazol-3-yl]oxy]propoxy]silane (B-2-8)

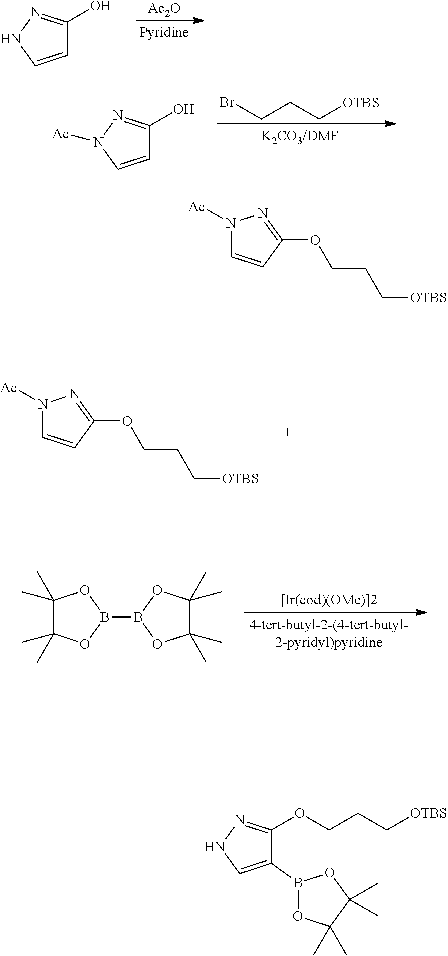

Step 1. To a mixture of 1H-pyrazol-3-ol (10.0 g, 119 mmol, 1 eq) in Pyridine (50 mL) was stirred at 95° C. for 0.5 h. The acetic anhydride (12.1 g, 119 mmol, 1 eq) in pyridine (50 mL) was added to the mixture reaction. The reaction was stirred at 95° C. for 2.5 h. On completion, the reaction mixture was concentrated in vacuo. The residue was diluted with water (80 mL) and extracted with EtOAc (3×120 mL). The combined organic layers were dried over Na₂SO₄, filtered and concentrated in vacuo to give a residue, which was triturated with PE (30 ml), filtered and dried under vacuo to afford 1-(3-hydroxypyrazol-1-yl)ethanone (12.0 g, 95.15 mmol, 80.00% yield) as yellow solid. ¹H NMR (400 MHz, DMSO-d₆) δ=10.97 (s, 1H), 8.13 (s, 1H), 6.00 (s, 1H), 2.47 (s, 3H).

Step 2. To a mixture of 1-(3-hydroxypyrazol-1-yl)ethanone (12.0 g, 95.2 mmol, 1 eq) and 3-bromopropoxy-tert-butyl-dimethyl-silane (26.5 g, 105 mmol, 1.1 eq) in DMF (100 mL) was added K₂CO₃ (39.4 g, 285 mmol, 3 eq). The reaction mixture was stirred at 60° C. for 4 h. On completion, the residue was diluted with water (300 mL) and extracted with EA (3×300 mL). The combined organic layers were dried over Na₂SO₄, filtered and concentrated in vacuo. The residue was purified by column chromatography to afford 1-[3-[3-[tertbutyl(dimethyl)silyl]oxypropoxy]pyrazol-1-yl]ethanone (24.0 g, 80.4 mmol, 84.5% yield) as yellow oil. LCMS: m/z 299.4 (M+1).

Step 3. To a mixture of 1-[3-[3-[tertbutyl(dimethyl)silyl]oxypropoxy]pyrazol-1-yl]ethenone (5.00 g, 16.7 mmol, 1 eq) and 4,4,5,5-tetramethyl-2-(4,4,5,5-tetramethyl-1,3,2-dioxaborolan-2-yl)-1,3,2-dioxaborolane (12.8 g, 50.3 mmol, 3 eq) in THF (50 mL) was added 4-tert-butyl-2-(4-tert-butyl-2-pyridyl)pyridine (899 mg, 3.35 mmol, 0.2 eq) and (1Z,5Z)-cycloocta-1,5-diene;2,4-dimethyl-BLAHbicyclo[1.1.0]butane (1.11 g, 1.68 mmol, 0.1 eq). The reaction mixture was stirred at 90° C. for 12 h. On completion, the residue was diluted with water (50 mL) and extracted with EA (3×80 mL). The combined organic layer was dried over Na₂SO₄, filtered and concentrated in vacuo. The residue was purified by column chromatography to afford tert-butyl-dimethyl-[3-[[4-(4,4,5,5-tetramethyl-1,3,2-dioxaborolan-2-yl)-1H-pyrazol-3-yl]oxy]propoxy]silane (6.00 g, 15.7 mmol, 93.7% yield) as yellow solid. LCMS: m/z 383.1 (M+1)

### General Method B: Preparation of (18E)-8-methyl-N-(propan-2-yl)-8,9,11,12-tetrahydro-2H-3,5-ethenodipyrazolo[3′,4′:9,10;4″,3″:13,14][1,4]dioxacyclopentadecino[5,6-b]pyridine-16-carboxamide (Ex. 2)

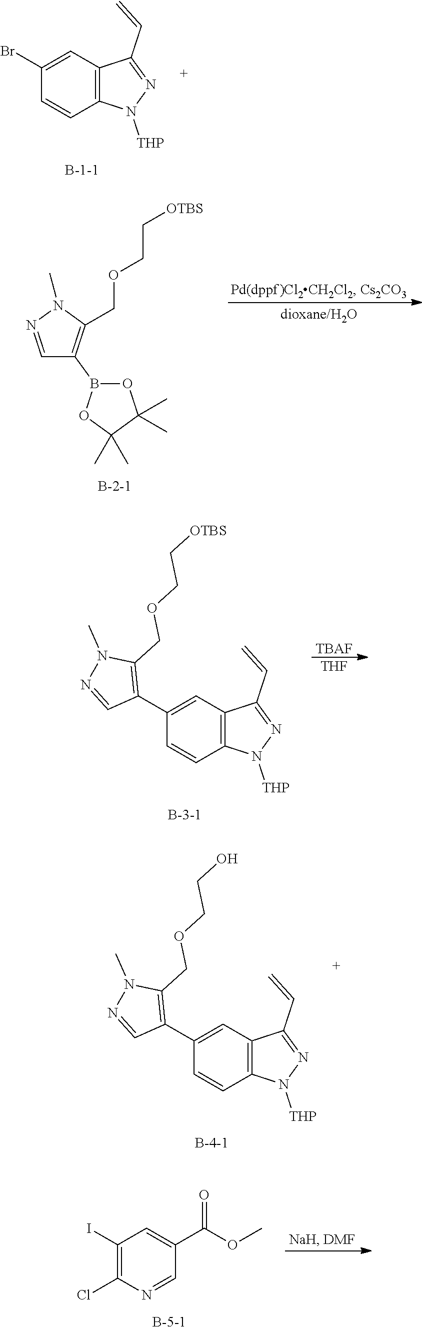

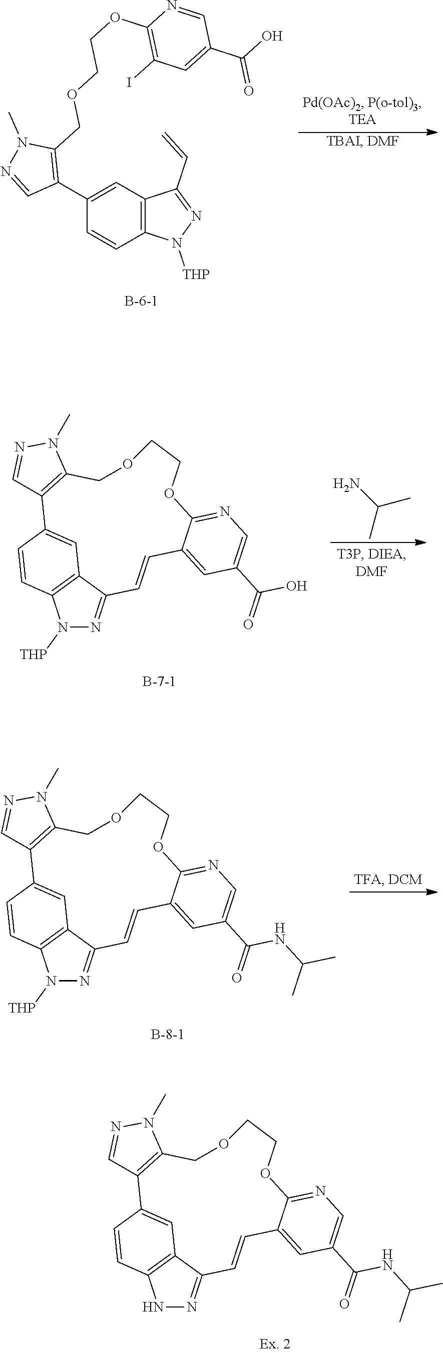

Step 1. To a solution of 5-bromo-1-tetrahydropyran-2-yl-3-vinyl-indazole (381 mg, 1.24 mmol, 1 eq) and tert-butyl-dimethyl-[2-[[2-methyl-4-(4,4,5,5-tetramethyl-1,3,2-dioxaborolan-2-yl)pyrazol-3-yl]methoxy]ethoxy]silane (1.25 g, 1.86 mmol, 59% purity, 1.5 eq) in mixture of solvent of dioxane (12 mL) and H₂O (3 mL) was added Cs₂CO₃ (1.21 g, 3.72 mmol, 3 eq) and ditertbutyl(cyclopentyl)phosphane;dichloropalladium;iron (80.8 mg, 124 umol, 0.1 eq).The mixture was stirred at 90° C. for 8 hr under N2. On completion, the mixture was filtered, diluted with H₂O (50 mL), extracted with EA (3×50 mL), washed with brine (3×50 mL), dried with anhydrous Na₂SO₄, filtered and concentrated in vacuo to afford tert-butyl-dimethyl-[2-[[2-methyl-4-(1-tetrahydropyran-2-yl-3-vinyl-indazol-5-yl)pyrazol-3-yl]methoxy]ethoxy]silane (1.1 g, 2.21 mmol, 89.2% yield) as a brown oil.

Step 2. To a mixture of tert-butyl-dimethyl-[2-[[2-methyl-4-(1-tetrahydropyran-2-yl-3-vinyl-indazol-5-yl)pyrazol-3-yl]methoxy]ethoxy]silane (1.20 g, 2.42 mmol, 1 eq) in THF (5 mL) was added TBAF (1 M, 3.62 mL, 1.5 eq) at 0° C. The reaction mixture was stirred at 25° C. for 1 hr. On completion, the residue was diluted with water (30 mL), then the residue was extracted with EA (3×70 mL). The combined organic layers was dried over Na₂SO₄, filtered and concentrated in vacuo to give a residue. The residue was purified by column chromatography to give 2-[[2-methyl-4-(1-tetrahydropyran-2-yl-3-vinyl-indazol-5-yl)pyrazol-3-yl]methoxy]ethanol (650 mg, 1.70 mmol, 70.3% yield) as yellow oil. ¹H NMR (400 MHz, DMSO-d₆) δ=7.98 (s, 1H), 7.78 (d, J=8.8 Hz, 1H), 7.70 (s, 1H), 7.54 (dd, J=1.2, 8.8 Hz, 1H), 7.03 (dd, J=11.6, 18.0 Hz, 1H), 6.12 (d, J=18.0 Hz, 1H), 5.85 (dd, J=2.4, 9.6 Hz, 1H), 5.53 (d, J=11.6 Hz, 1H), 4.58 (s, 2H), 3.90 (s, 4H), 3.80-3.71 (m, 2H), 3.62-3.50 (m, 5H), 1.98 (s, 3H), 1.17 (t, J=7.6 Hz, 2H).

Step 3. To a mixture of 2-∥2-methyl-4-(1-tetrahydropyran-2-yl-3-vinyl-indazol-5-yl)pyrazol-3-yl]methoxy]ethanol (50.0 mg, 0.130 mmol, 1 eq) in DMF (5 mL) was added NaH (7.84 mg, 0.196 mmol, 60% purity, 1.5 eq) at 0° C. for 30 min, then the methyl 6-chloro-5-iodo-pyridine-3-carboxylate (38.8 mg, 0.130 mmol, 1 eq) was added. The reaction mixture was stirred at 100° C. for 12 hrs. On completion, the residue was diluted with water (5 mL), then the residue was extracted with EA (3×10 mL). The combined organic layers were dried over Na₂SO₄, filtered and concentrated in vacuo. The residue was purified by prep-HPLC to give 5-iodo-6-[2-[[2-methyl-4-(1-tetrahydropyran-2-yl-3-vinyl-indazol-5-yl)pyrazol-3-yl]methoxy]ethoxy]pyridine-3-carboxylic acid (16.0 mg, 0.0254 mmol, 19% yield) as white solid. ¹H NMR (400 MHz, DMSO-d₆) δ=8.62 (d, J=2.0 Hz, 1H), 8.50 (d, J=2.0 Hz, 1H), 7.96 (s, 1H), 7.70 (s, 1H), 7.66 (d, J=8.8 Hz, 1H), 7.50 (dd, J=1.6, 8.8 Hz, 1H), 6.99 (dd, J=11.6, 18.0 Hz, 1H), 6.10 (dd, J=0.8, 18.0 Hz, 1H), 5.80 (dd, J=2.4, 9.6 Hz, 1H), 5.50-5.45 (m, 1H), 4.70 (s, 2H), 4.57-4.53 (m, 2H), 3.95-3.89 (m, 6H), 3.78-3.71 (m, 1H), 2.42-2.36 (m, 1H), 2.09-1.91 (m, 3H), 1.82-1.69 (m, 1H), 1.63-1.54 (m, 2H).

Step 4. To a mixture of 5-iodo-6-[2-[[2-methyl-4-(1-tetrahydropyran-2-yl-3-vinyl-indazol-5-yl)pyrazol-3-yl]methoxy]ethoxy]pyridine-3-carboxylic acid (16.0 mg, 0.0254 mmol, 1 eq) in DMF (1 mL) was added Pd(OAc)₂ (2.85 mg, 0.127 mmol, 0.5 eq), TEA (10.2 mg, 0.101 mmol, 4 eq),TBAI (0.94 mg, 0.00254 mmol, 0.1 eq) and P(o-tolyl)₃ (3.87 mg, 0.012.7 mmol, 0.5 eq). The reaction mixture was stirred at 60° C. for 12 hrs. On completion, the reaction mixture was filtered and concentrated in vacuo. The residue was purified by prep-HPLC to give B-7-1 (12.0 mg, 23.9 umol, 94.1% yield) as white solid. ¹H NMR (400 MHz, DMSO-d₆) δ=13.32-12.85 (m, 1H), 8.65 (d, J=2.0 Hz, 1H), 8.57 (d, J=2.0 Hz, 1H), 8.28 (s, 1H), 8.05 (d, J=17.2 Hz, 1H), 7.84 (d, J=8.8 Hz, 1H), 7.70 (s, 1H), 7.64-7.54 (m, 2H), 5.91 (dd, J=2.4, 9.2 Hz, 1H), 4.66 (s, 2H), 4.60-4.55 (m, 2H), 4.05-4.00 (m, 2H), 3.95-3.87 (m, 4H), 3.80-3.74 (m, 1H), 2.10-1.98 (m, 3H), 1.81-1.72 (m, 1H), 1.63-1.62 (m, 2H).

Step 5 To a mixture of B-7-1 (6.00 mg, 0.0119 mmol, 1 eq) in DCM (1 mL) were added T₃P (4.57 mg, 0.0143 mmol, 1.2 eq), DIEA (4.64 mg, 0.0358 mmol, 3 eq), and a solution of propan-2-amine (1.41 mg, 0.0239 mmol, 2 eq) in DCM (1 mL). The reaction mixture was stirred at 25° C. for 1 hr. On completion, the residue was quenched with water (0.5 mL) and extracted with DCM (3×5 mL). The combined organic layers were dried over Na₂SO₄, filtered and concentrated in vacuo to give B-8-1 (6.00 mg, 0.011 mmol, 92.4% yield) as yellow oil.

Step 6. To a mixture of B-8-1 (6 mg, 0.011 mmol, 1 eq) in DCM (0.5 mL) was added TFA (770 mg, 6.75 mmol, 610 eq). The reaction mixture was stirred at 25° C. for 0.5 hr. On completion, the reaction mixture was concentrated in vacuo. The residue was purified by prep-HPLC to give Ex. 2 (1.84 mg, 0.0032 mmol, 28.7% yield) as an off-white solid.

Ex. 3-Ex. 5 were prepared using General Method B with corresponding amines for the amide formation.

### Preparation of 2-(5-bromo-1-tetrahydropyran-2-yl-indazol-3-yl)ethynyl-triisopropyl-silane (C-1-1)

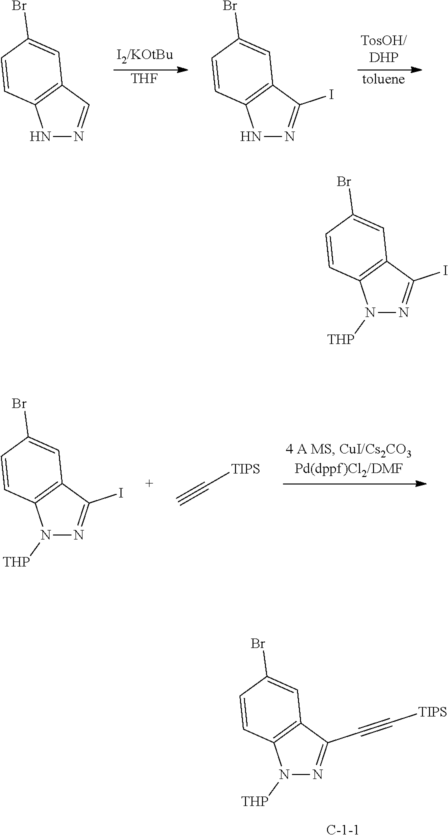

Step 1. A solution of 5-bromo-1H-indazole (21.0 g, 107 mmol, 1 eq) in THE (250 mL) was cooled down on an ice bath and KOtBu (35.9 g, 320 mmol, 3 eq) was added portion wise. The resulting slurry was stirred at 0° C. and a solution of I₂ (54.1 g, 213 mmol, 42.9 mL, 2 eq) in THE (250 mL) was added dropwise. The mixture was stirred at 25° C. for 12 hours. On completion, the reaction mixture was filtered and the filtrate was diluted with H₂O (20 mL) and extracted with EtOAc (20 mL*3). The combined organic layers were washed with brine (20 mL*3), dried over Na₂SO₄, filtered and concentrated under reduced pressure. The residue was purified by flash silica gel chromatography to afford 5-bromo-3-iodo-1H-indazole (120 g, 350 mmol, 82% yield, 94% purity) as a white solid. LCMS: 324.7 (M+1).

Step 2. To a mixture of 5-bromo-3-iodo-1H-indazole (25.0 g, 77.4 mmol, 1 eq) and 3,4-dihydro-2H-pyran (13.0 g, 155 mmol, 2 eq) in toluene (250 mL) was added 4-methylbenzenesulfonic acid (2.67 g, 15.5 mmol, 0.2 eq). The mixture was stirred at 90° C. for 12 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford 5-bromo-3-iodo-1-tetrahydropyran-2-yl-indazole (24.0 g, 58.9 mmol, 76% yield) as a white solid. ¹H NMR (400 MHz, CDCl₃) δ=7.64 (d, J=1.6 Hz, 1H), 7.54-7.44 (m, 2H), 5.68 (dd, J=3.2, 9.1 Hz, 1H), 4.04-3.95 (m, 1H), 3.79-3.66 (m, 1H), 2.58-2.46 (m, 1H), 2.20-2.03 (m, 2H), 1.87-1.54 (m, 3H).

Step 3. To a mixture of 5-bromo-3-iodo-1-tetrahydropyran-2-yl-indazole (23.0 g, 56.5 mmol, 1 eq) and ethynyl(triisopropyl)silane (11.3 g, 62.2 mmol, 1.1 eq) in DMF (250 mL) was added Cs₂CO₃ (55.2 g, 170 mmol, 3 eq), Pd(dppf)Cl₂ (2.48 g, 3.39 mmol, 0.06 eq) and CuI (646 mg, 3.39 mmol, 0.06 eq) under N2. The mixture was stirred at 25° C. for 3 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford compound 2-(5-bromo-1-tetrahydropyran-2-yl-indazol-3-yl)ethynyl-triisopropyl-silane (II-1-1, 38.0 g, 79.9 mmol, 70% yield) as a white solid. ¹H NMR (400 MHz, CDCl₃) δ=7.87 (s, 1H), 7.57-7.43 (m, 2H), 5.70 (dd, J=2.4, 9.2 Hz, 1H), 4.02 (bd, J=11.2 Hz, 1H), 3.80-3.65 (m, 1H), 2.59-2.41 (m, 1H), 2.14 (d, J=3.2 Hz, 1H), 2.08 (s, 1H), 1.79-1.70 (m, 2H), 1.67 (s, 1H), 1.22-1.18 (m, 18H), 1.18-1.14 (m, 3H).

### Preparation of 2-(5-bromo-6-fluoro-1-tetrahydropyran-2-yl-indazol-3-yl)ethynyl-triisopropyl-silane (C-1-2)

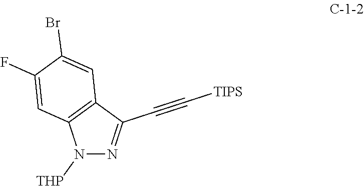

C-1-2 was prepared following similar methods as C-1-1 using 5-bromo-6-fluoro-1H-indazole as starting material.

### Preparation of Ethyl 2-(5-hydroxy-3-methyl-pyrazol-1-yl) acetate (C-6-6)

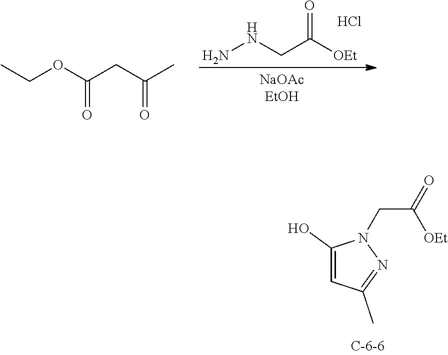

To a solution of ethyl 2-hydrazinoacetate;hydrochloride (19.0 g, 123 mmol, 1 eq) in EtOH (200 mL) were added NaOAc (10.1 g, 123 mmol, 1 eq) and ethyl 3-oxobutanoate (14.3 g, 123 mmol, 13.2 mL, 1 eq). The mixture was stirred at 80° C. for 2 h. The reaction mixture was cooled and concentrated under reduced pressure. The residue was purified by column chromatography to provide ethyl 2-(5-hydroxy-3-methyl-pyrazol-1-yl) acetate (16.0 g, 86.9 mmol, 70.7% yield) as white solid. ¹H NMR (400 MHz, CHLOROFORM-d) δ=4.41 (s, 2H), 4.27-4.14 (m, 2H), 3.25 (s, 2H), 2.11 (s, 3H), 1.28 (d, J=7.2 Hz, 3H).

### Preparation of Ethyl 2-(5-hydroxy-3-ethyl-pyrazol-1-yl) acetate (C-6-7)

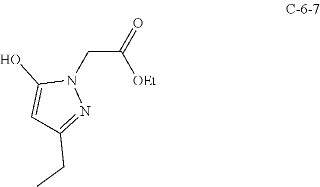

C-6-7 was prepared with the same method as C-6-6 using ethyl 3-oxopentanoate as starting material.

### Preparation of ethyl 2-(3-hydroxy-5-methyl-pyrazol-1-yl)acetate (C-6-8)

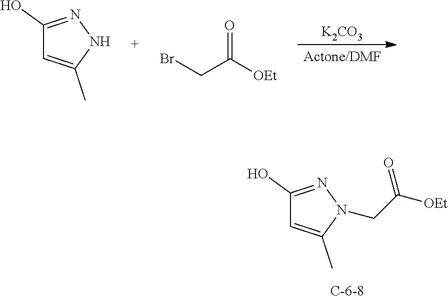

To a mixture of 5-methyl-1,2-dihydropyrazol-3-one (20.0 g, 204 mmol, 1 eq) and ethyl 2-bromoacetate (30.6 g, 183 mmol, 0.9 eq) in DMF (60 mL) and ACETONE (240 mL) was added K₂CO₃ (28.2 g, 204 mmol, 1 eq). The mixture was stirred at 20° C. for 3 h. On completion, the filter liquor was diluted with H₂O (30 mL) and extracted with EtOAc (30 mL*3). The combined organic layers were washed with brine (30 mL*3), dried over Na₂SO₄, filtered and concentrated under reduced pressure. The residue was purified by flash silica gel chromatography to provide ethyl 2-(3-hydroxy-5-methyl-pyrazol-1-yl)acetate (10.0 g, 54.3 mmol, 26.6% yield) was as a white solid. ¹H NMR (400 MHz, CDCl₃) δ=5.47 (s, 1H), 4.58 (s, 2H), 4.22 (q, J=7.2 Hz, 2H), 2.17 (s, 3H), 1.28 (t, J=7.2 Hz, 3H).

### General Method C: Preparation of (17E)-8,14,16-trimethyl-2,8,9,11,12,14-hexahydro-3,5-etheno[1,4]dioxacyclopentadecino[11,10-c:15,14-c′:6,7-c″ ]tripyrazole (Ex. 6)

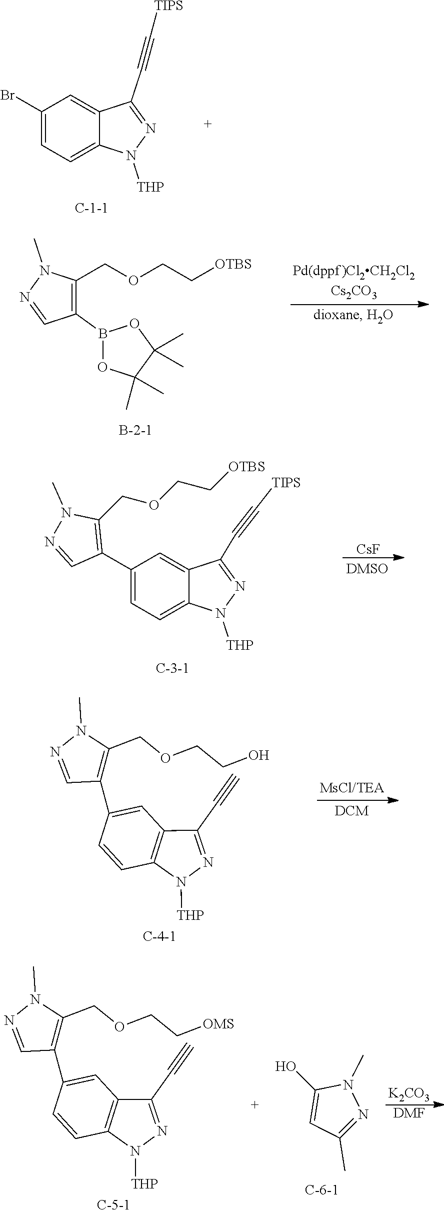

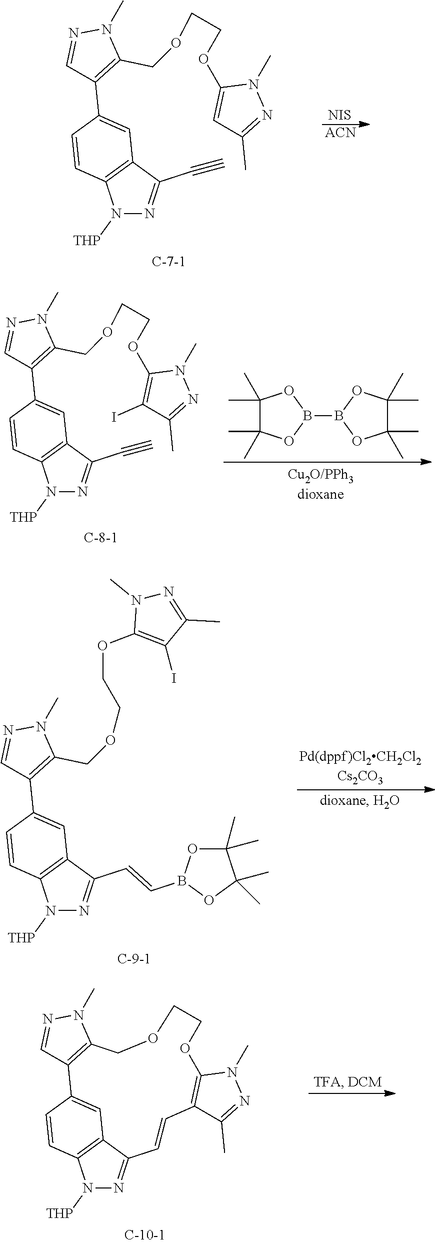

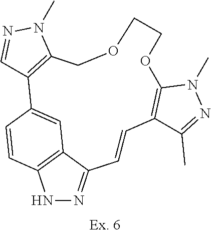 [SMILES: Cc1nn(C)c2c1/C=C/c1n[nH]c3ccc(cc13)-c1cnn(C)c1COCCO2]

Step 1. A mixture of tert-butyl-dimethyl-[2-[[2-methyl-4-(4,4,5,5-tetramethyl-1,3,2-dioxaborolan-2-yl)pyrazol-3-yl]methoxy]ethoxy]silane (11.7 g, 29.5 mmol, 1 eq), 2-(5-bromo-1-tetrahydropyran-2-yl-indazol-3-yl)ethynyl-triisopropyl-silane (15.0 g, 32.5 mmol, 1.1 eq), Pd(dppf)Cl₂·CH₂Cl₂ (2.41 g, 2.95 mmol, 0.1 eq), Cs₂CO₃ (28.9 g, 88.6 mmol, 3 eq) in dioxane (100 mL) and H₂O (20 mL) was degassed and purged with N2 for 3 times, and then the mixture was stirred at 90° C. for 2 h under N2 atmosphere. After cooling down, the reaction mixture was filtered and the filtrate was concentrated under reduced pressure to give a residue. The residue was purified by flash silica gel chromatography to give tert-butyl-dimethyl-[2-[[2-methyl-4-[1-tetrahydropyran-2-yl-3-(2-triisopropylsilylethynyl) indazol-5-yl]pyrazol-3-yl]methoxy]ethoxy]silane (14.2 g, 21.8 mmol, 73.9% yield) as a yellow oil. LCMS: m/z 651.8 (M+1)

Step 2. A mixture of tert-butyl-dimethyl-[2-[[2-methyl-4-[1-tetrahydropyran-2-yl-3-(2-triisopropylsilylethynyl) indazol-5-yl]pyrazol-3-yl]methoxy]ethoxy]silane (1.00 g, 1.54 mmol, 1 eq) and CsF (2.10 g, 13.8 mmol, 9 eq) in DMSO (10 mL) at 25° C. for 38 h. The mixture was diluted with H₂O (40 mL) and extracted with EtOAc (20 mL*3). The combined organic layer was dried over anhydrous Na₂SO₄, filtered and the filtrate was concentrated in vacuum. The residue was purified by combi flash to give 2-[[4-(3-ethynyl-1-tetrahydropyran-2-yl-indazol-5-yl)-2-methyl-pyrazol-3-yl]methoxy]ethanol (500 mg, 1.31 mmol, 85.6% yield) was obtained as a brown oil. LCMS: m/z 381.1 (M+1).

Step 3. To a mixture of 2-[[4-(3-ethynyl-1-tetrahydropyran-2-yl-indazol-5-yl)-2-methyl-pyrazol-3-yl]methoxy]ethanol (500 mg, 1.31 mmol, 1 eq) in DCM (5 mL) was added TEA (665 mg, 6.57 mmol, 5 eq). The mixture was stirred at 0° C. followed by addition of MsCl (452 mg, 3.94 mmol, 3 eq). The mixture was stirred at 20° C. for 1 hr, and quenched with sat. aqueous NaHCO₃ (20 mL) and extracted with DCM (20 mL*3). The combined organic layer was dried over anhydrous Na₂SO₄, filtered and the filtrate was concentrated in vacuum to give crude 2-[[4-(3-ethynyl-1-tetrahydropyran-2-yl-indazol-5-yl)-2-methyl-pyrazol-3-yl]methoxy]ethyl methanesulfonate (580 mg, 1.26 mmol, 96.2% yield) as a brown oil. LCMS: m/z 459.1 (M+1).

Step 4. To a mixture of 2-[[4-(3-ethynyl-1-tetrahydropyran-2-yl-indazol-5-yl)-2-methyl-pyrazol-3-yl]methoxy]ethyl methanesulfonate (560 mg, 1.22 mmol, 1 eq) and 2,5-dimethyl-4H-pyrazol-3-one (151 mg, 1.34 mmol, 1.1 eq) in DMF (14 mL) was added K₂CO₃ (506 mg, 3.66 mmol, 3 eq). The reaction mixture was stirred at 60° C. for 12 h. The mixture was diluted with water (20 mL), and then extracted with EtOAc (3 * 20 mL). The combined organic layers were dried over Na₂SO₄, filtered and concentrated in vacuo. The residue was purified by column chromatography to give 5-[5-[2-(2,5-dimethylpyrazol-3-yl)oxyethoxymethyl]-1-methyl-pyrazol-4-yl]-3-ethynyl-1-tetrahydropyran-2-yl-indazole (370 mg, 780 umol, 63.8% yield) as a brown oil. LCMS: m/z 475.0 (M+1).

Step 5. To a solution of 5-[5-[2-(2,5-dimethylpyrazol-3-yl)oxyethoxymethyl]-1-methyl-pyrazol-4-yl]-3-ethynyl-1-tetrahydropyran-2-yl-indazole (360 mg, 0.759 mmol, 1 eq) in ACN (11 mL) was added NIS (154 mg, 0.683 mmol, 0.9 eq) at 0° C. The mixture was stirred at 0° C. for 1 h. The reaction mixture was quenched by addition of aqueous Na₂SO₃ solution (1 M, 15 mL) at 20° C., and extracted with EtOAc (30 mL*3). The combined organic layer was dried over anhydrous Na₂SO₄, filtered and the filtrate was concentrated in vacuum. The residue was purified by column chromatography to give 3-ethynyl-5-[5-[2-(4-iodo-2,5-dimethyl-pyrazol-3-yl)oxyethoxymethyl]-1-methyl-pyrazol-4-yl]-1-tetrahydropyran-2-yl-indazole (402 mg, 0.670 mmol, 88.3% yield) as a brown oil. LCMS: m/z 600.9 (M+1).

Step 6. To a mixture of 4,4,5,5-tetramethyl-2-(4,4,5,5-tetramethyl-1,3,2-dioxaborolan-2-yl)-1,3,2-dioxaborolane (296 mg, 1.17 mmol, 2 eq) and PPh₃(168 mg, 0.641 mmol, 1.1 eq) and cupriooxycopper (41.7 mg, 0.291 mmol, 0.5 eq) and 3-ethynyl-5-[5-[2-(4-iodo-2,5-dimethyl-pyrazol-3-yl)oxyethoxymethyl]-1-methyl-pyrazol-4-yl]-1-tetrahydropyran-2-yl-indazole (350 mg, 0.583 mmol, 1 eq) in dioxane (10.5 mL). The reaction mixture was stirred at 60° C. for 16 hrs. The mixture was concentrated in vacuum and the residue was purified by column chromatography to give {5-[5-[2-(4-iodo-2,5-dimethyl-pyrazol-3-yl)oxyethoxymethyl]-1-methyl-pyrazol-4-yl]-1-tetrahydropyran-2-yl-3-[(E)-2-(4,4,5,5-tetramethyl-1,3,2-dioxaborolan-2-yl)vinyl]indazole (450 mg, crude) as a brown oil. ¹H NMR (400 MHz, CDCl₃) δ=7.97 (s, 1H), 7.77-7.71 (m, 1H), 7.63-7.60 (m, 2H), 7.42 (dd, J=1.6, 8.4 Hz, 1H), 6.50 (d, J=18.8 Hz, 1H), 5.75 (dd, J=2.8, 8.8 Hz, 1H), 4.68 (s, 2H), 4.37-4.34 (m, 2H), 4.18-4.08 (m, 1H), 4.00 (s, 2H), 4.03-3.98 (m, 1H), 3.79-3.73 (m, 3H), 3.59 (s, 3H), 3.23-3.20 (m, 1H), 2.65-2.50 (m, 1H), 2.26-2.18 (m, 1H), 2.16 (s, 3H), 2.13-2.07 (m, 1H), 2.05 (s, 1H), 1.84-1.68 (m, 3H), 1.58 (s, 2H), 1.31 (s, 12H)

Step 7. A mixture of 5-[5-[2-(4-iodo-2,5-dimethyl-pyrazol-3-yl)oxyethoxymethyl]-1-methyl-pyrazol-4-yl]-1-tetrahydropyran-2-yl-3-[(E)-2-(4,4,5,5-tetramethyl-1,3,2-dioxaborolan-2-yl)vinyl]indazole (400 mg, 0.549 mmol, 1 eq), Cs₂CO₃ (537 mg, 1.65 mmol, 3 eq), Pd(dppf)Cl₂·CH₂Cl₂ (44.8 mg, 0.0549 mmol, 0.1 eq) in dioxane (4 mL) and H₂O (0.8 mL) was degassed and purged with N₂ for 3 times, and then the mixture was stirred at 80° C. for 2 h under N₂ atmosphere. The mixture was concentrated in vacuum and the residue was purified by column chromatography to give C-10-1 (150 mg, 0.316 mmol, 57.6% yield) as a brown oil. LCMS: m/z 475.3 (M+1).

Step 8. To a solution of C-10-1 (140 mg, 0.295 mmol, 1 eq) in DCM (7 mL) was added TFA (7 mL). The mixture was stirred at 20° C. for 1 h and then concentrated in vacuum. The residue was purified by column chromatography to give Ex. 6 (20.0 mg, 0.0487 mmol, 16.5% yield) as a brown solid.

Ex. 6-Ex. 20 were prepared with General Method C using corresponding C-1-X, B-2-X, and C-6-X as shown below. C-6-X compounds are commercially available or made via conventional methods. Ex. 19 and Ex. 20 were obtained as regio-isomers without iodination step (Step 5).

| C-1-X | B-2-X | C-6-X | Ex. # |
|---|---|---|---|
| C-1-1 | B-2-1 | C-6-1 | Ex. 6 |
| 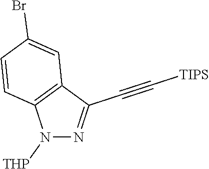 | 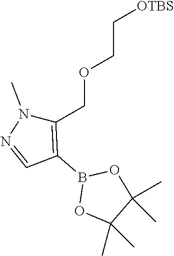 [SMILES: Cn1ncc(B2OC(C)(C)C(C)(C)O2)c1COCCO[Si](C)(C)C(C)(C)C] | 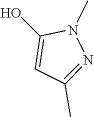 | 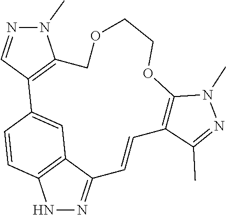 [SMILES: Cc1nn(C)c2c1/C=C/c1n[nH]c3ccc(cc13)-c1cnn(C)c1COCCO2] |
| C-1-1 | B-2-1 | C-6-2 | Ex. 7 |
| 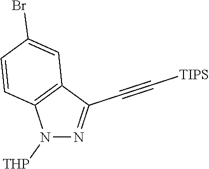 | 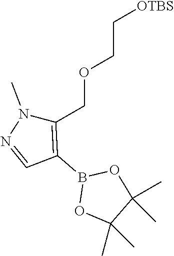 [SMILES: Cn1ncc(B2OC(C)(C)C(C)(C)O2)c1COCCO[Si](C)(C)C(C)(C)C] | 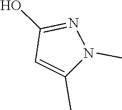 | 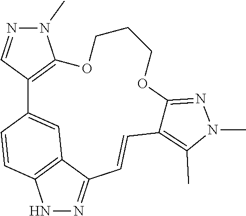 [SMILES: Cc1c2c(nn1C)OCCCOc1c(cnn1C)-c1ccc3[nH]nc(c3c1)/C=C/2] |
| C-1-1 | B-2-2 | C-6-1 | Ex. 8 |
| 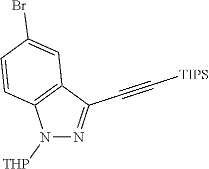 | 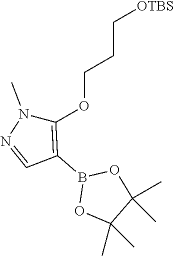 [SMILES: Cn1ncc(B2OC(C)(C)C(C)(C)O2)c1OCCCO[Si](C)(C)C(C)(C)C] | 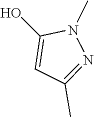 | 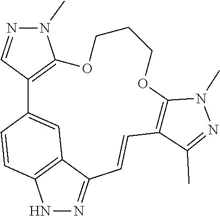 [SMILES: Cc1nn(C)c2c1/C=C/c1n[nH]c3ccc(cc13)-c1cnn(C)c1OCCCO2] |
| C-1-1 | B-2-2 | C-6-2 | Ex. 9 |
| 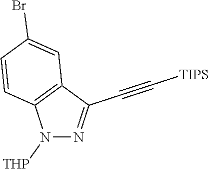 | 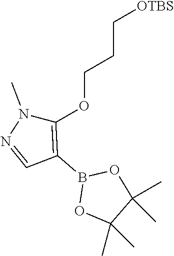 [SMILES: Cn1ncc(B2OC(C)(C)C(C)(C)O2)c1OCCCO[Si](C)(C)C(C)(C)C] | 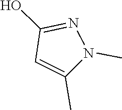 | 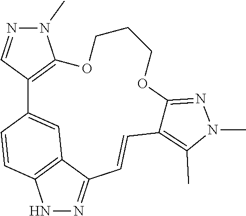 [SMILES: Cc1c2c(nn1C)OCCCOc1c(cnn1C)-c1ccc3[nH]nc(c3c1)/C=C/2] |
| C-1-1 | B-2-2 | C-6-3 | Ex. 10 |
| 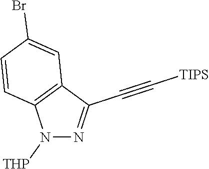 | 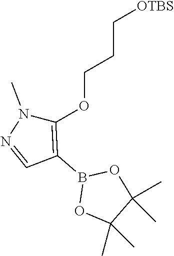 [SMILES: Cn1ncc(B2OC(C)(C)C(C)(C)O2)c1OCCCO[Si](C)(C)C(C)(C)C] | 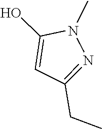 | 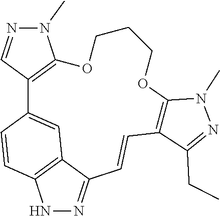 [SMILES: CCc1nn(C)c2c1/C=C/c1n[nH]c3ccc(cc13)-c1cnn(C)c1OCCCO2] |
| C-1-1 | B-2-2 | C-6-4 | Ex. 11 |
| 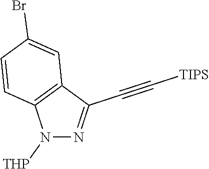 | 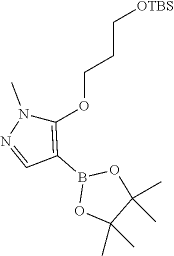 [SMILES: Cn1ncc(B2OC(C)(C)C(C)(C)O2)c1OCCCO[Si](C)(C)C(C)(C)C] | 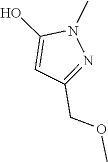 | 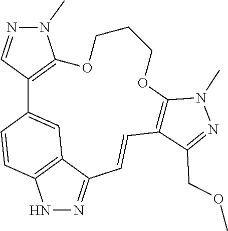 [SMILES: COCc1nn(C)c2c1/C=C/c1n[nH]c3ccc(cc13)-c1cnn(C)c1OCCCO2] |
| C-1-2 | B-2-2 | C-6-1 | Ex. 12 |
| 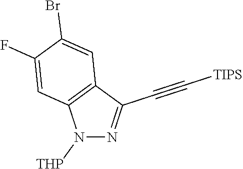 | 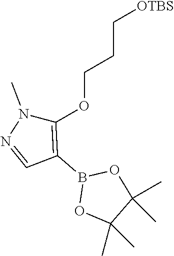 [SMILES: Cn1ncc(B2OC(C)(C)C(C)(C)O2)c1OCCCO[Si](C)(C)C(C)(C)C] | 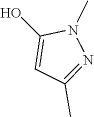 | 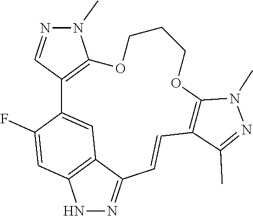 [SMILES: Cc1nn(C)c2c1/C=C/c1n[nH]c3cc(F)c(cc13)-c1cnn(C)c1OCCCO2] |
| C-1-1 | B-2-3 | C-6-3 | Ex. 13 |
| 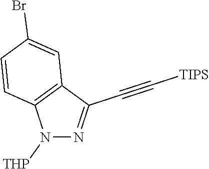 | 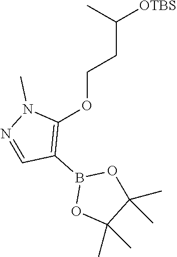 [SMILES: CC(CCOc1c(B2OC(C)(C)C(C)(C)O2)cnn1C)O[Si](C)(C)C(C)(C)C] | 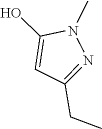 | 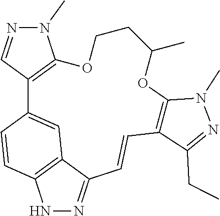 [SMILES: CCc1nn(C)c2c1/C=C/c1n[nH]c3ccc(cc13)-c1cnn(C)c1OCCC(C)O2] |
| C-1-1 | B-2-3 | C-6-3 | Ex. 14 |
| 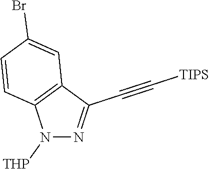 | 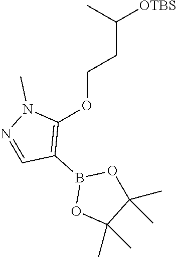 [SMILES: CC(CCOc1c(B2OC(C)(C)C(C)(C)O2)cnn1C)O[Si](C)(C)C(C)(C)C] | 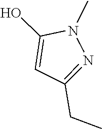 | 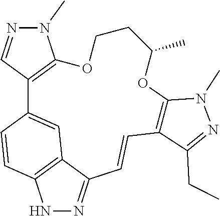 [SMILES: CCc1nn(C)c2c1/C=C/c1n[nH]c3ccc(cc13)-c1cnn(C)c1OCC[C@H](C)O2] |
| C-1-1 | B-2-3 | C-6-3 | Ex. 15 |
| 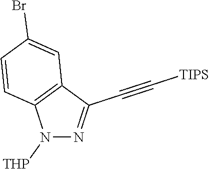 | 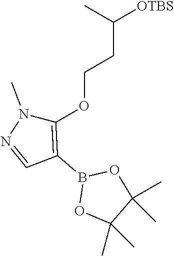 [SMILES: CC(CCOc1c(B2OC(C)(C)C(C)(C)O2)cnn1C)O[Si](C)(C)C(C)(C)C] | 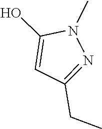 | 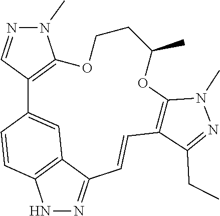 [SMILES: CCc1nn(C)c2c1/C=C/c1n[nH]c3ccc(cc13)-c1cnn(C)c1OCC[C@@H](C)O2] |
| C-1-1 | B-2-4 | C-6-5 | Ex. 16 |
| 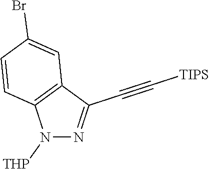 | 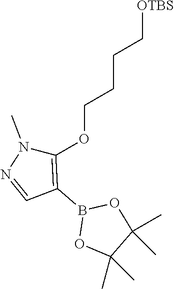 [SMILES: Cn1ncc(B2OC(C)(C)C(C)(C)O2)c1OCCCCO[Si](C)(C)C(C)(C)C] | 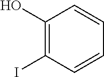 | 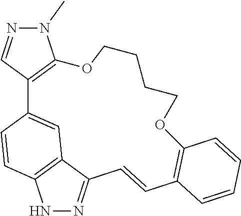 [SMILES: Cn1ncc2c1OCCCCOc1ccccc1/C=C/c1n[nH]c3ccc-2cc13] |
| C-1-1 | B-2-5 | C-6-1 | Ex. 17 |
| 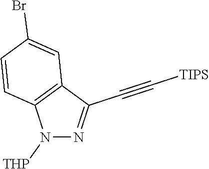 | 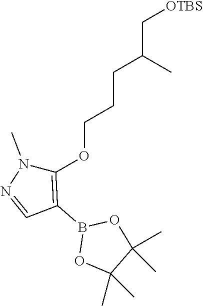 [SMILES: CC(CCCOc1c(B2OC(C)(C)C(C)(C)O2)cnn1C)CO[Si](C)(C)C(C)(C)C] | 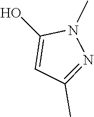 | 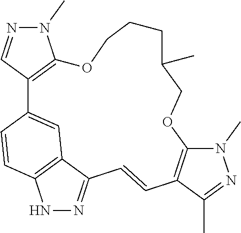 [SMILES: Cc1nn(C)c2c1/C=C/c1n[nH]c3ccc(cc13)-c1cnn(C)c1OCCCC(C)CO2] |
| C-1-1 | B-2-6 | C-6-1 | Ex. 18 |
| 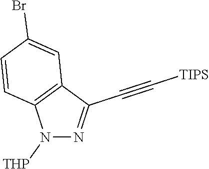 | 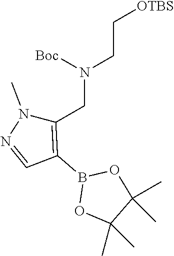 [SMILES: Cn1ncc(B2OC(C)(C)C(C)(C)O2)c1CN(CCO[Si](C)(C)C(C)(C)C)C(=O)OC(C)(C)C] | 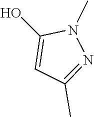 | 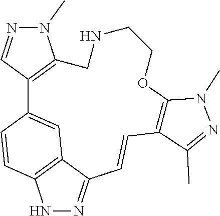 [SMILES: Cc1nn(C)c2c1/C=C/c1n[nH]c3ccc(cc13)-c1cnn(C)c1CNCCO2] |
| C-1-1 | B-2-4 | C-6-6 | Ex. 19 |
| 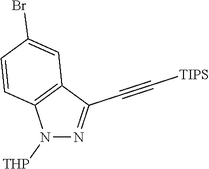 | 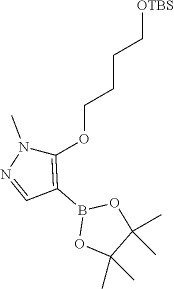 [SMILES: Cn1ncc(B2OC(C)(C)C(C)(C)O2)c1OCCCCO[Si](C)(C)C(C)(C)C] | 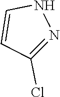 | 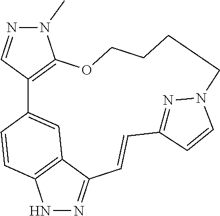 [SMILES: Cn1ncc2c1OCCCCn1ccc(n1)/C=C/c1n[nH]c3ccc-2cc13] |
| C-1-1 | B-2-4 | C-6-6 | Ex. 20 |
| 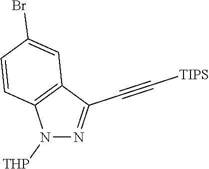 | 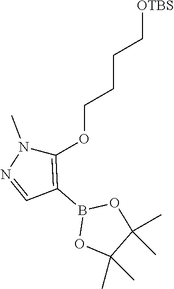 [SMILES: Cn1ncc(B2OC(C)(C)C(C)(C)O2)c1OCCCCO[Si](C)(C)C(C)(C)C] | 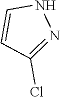 | 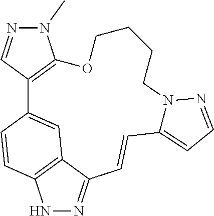 [SMILES: Cn1ncc2c1OCCCCn1nccc1/C=C/c1n[nH]c3ccc-2cc13] |

### General Method D: Preparation of 2-[(17E)-8,9,16-trimethyl-8,9,11,12-tetrahydro-3,5-etheno[1,4]dioxacyclopentadecino[11,10-c:15,14-c′:6,7-c″]tripyrazol-14(2H-yl]ethan-1-ol (Ex. 21)

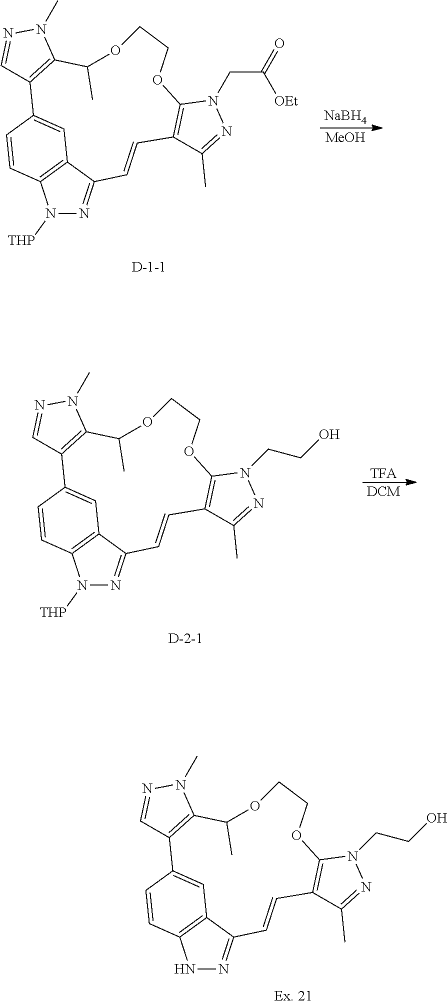

D-1-1 was prepared with General Method C.

Step 1. To a solution of D-1-1 (70.00 mg, 0.125 mmol, 1 eq) in MeOH (0.9 mL) was added NaBH₄ (47.24 mg, 1.25 mmol, 10 eq) at 0° C. The mixture was stirred at 25° C. for 2 h and then quenched with water (2 mL) and extracted with ethyl acetate (10 mL×3). The combined organic phase was dried over anhydrous sodium sulfate, filtered and concentrated. The residue was purified by prep-TLC (Petroleum ether:Ethyl acetate=1:1) to give D-2-1 (58 mg, 0.11 mmol, 89.57% yield) as a yellow oil. LCMS: m/z 519.1 (M+1)

Step 2. To a solution of D-2-1 (18.0 mg, 0.0347 mmol, 1 eq) in DCM (0.6 mL) was added TFA (0.66 mL). The mixture was stirred at 25° C. for 2 h and concentrated. The residue was purified by reverse-phase prep-HPLC to give Ex. 21 (2.32 mg, 0.005 mmol, 14.6% yield) as a white solid.

Ex. 21-Ex. 27 were prepared with General Method C&D using C-1-1, and the corresponding B-2-X, and C-6-X as shown below.

| B-2-X | C-6-X | D-1-X | Ex. # |
|---|---|---|---|
| B-2-7 | C-6-6 | D-1-1 | Ex. 21 |
| 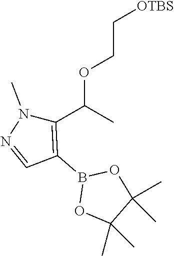 [SMILES: CC(OCCO[Si](C)(C)C(C)(C)C)c1c(B2OC(C)(C)C(C)(C)O2)cnn1C] | 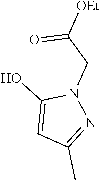 | 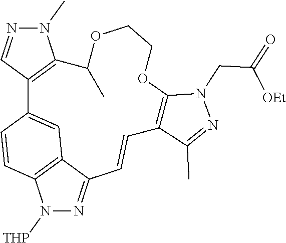 | 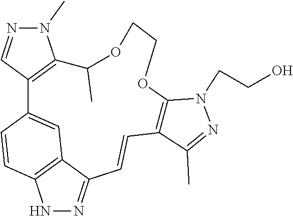 [SMILES: Cc1nn(CCO)c2c1/C=C/c1n[nH]c3ccc(cc13)-c1cnn(C)c1C(C)OCCO2] |
| B-2-2 | C-6-6 | D-1-2 | Ex. 22 |
| 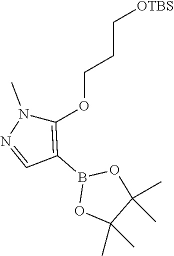 [SMILES: Cn1ncc(B2OC(C)(C)C(C)(C)O2)c1OCCCO[Si](C)(C)C(C)(C)C] | 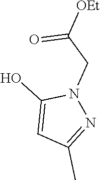 | 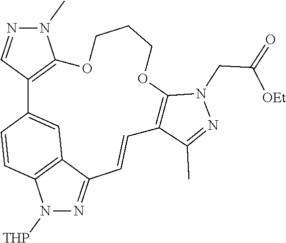 | 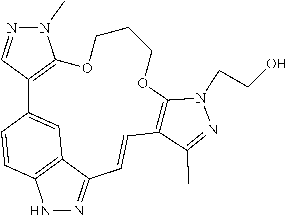 [SMILES: Cc1nn(CCO)c2c1/C=C/c1n[nH]c3ccc(cc13)-c1cnn(C)c1OCCCO2] |
| B-2-2 | C-6-7 | D-1-3 | Ex. 23 |
| 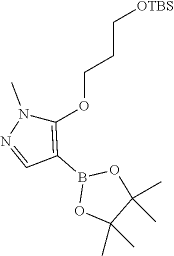 [SMILES: Cn1ncc(B2OC(C)(C)C(C)(C)O2)c1OCCCO[Si](C)(C)C(C)(C)C] | 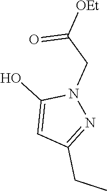 | 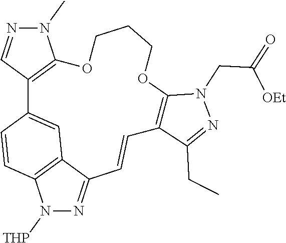 | 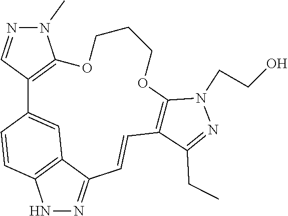 [SMILES: CCc1nn(CCO)c2c1/C=C/c1n[nH]c3ccc(cc13)-c1cnn(C)c1OCCCO2] |
| B-2-2 | C-6-8 | D-1-4 | Ex. 24 |
| 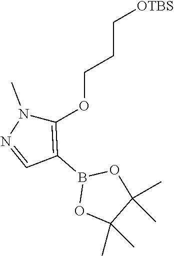 [SMILES: Cn1ncc(B2OC(C)(C)C(C)(C)O2)c1OCCCO[Si](C)(C)C(C)(C)C] | 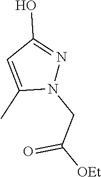 | 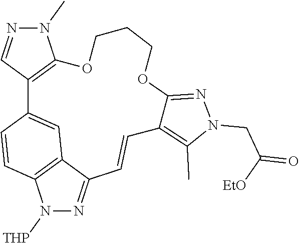 | 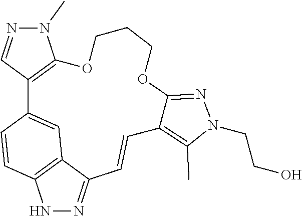 [SMILES: Cc1c2c(nn1CCO)OCCCOc1c(cnn1C)-c1ccc3[nH]nc(c3c1)/C=C/2] |
| B-2-5 | C-6-6 | D-1-4 | Ex. 25 |
| 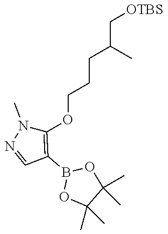 [SMILES: CC(CCCOc1c(B2OC(C)(C)C(C)(C)O2)cnn1C)CO[Si](C)(C)C(C)(C)C] | 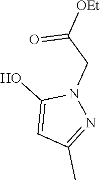 | 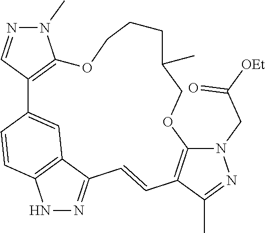 [SMILES: CCOC(=O)Cn1nc(C)c2c1OCC(C)CCCOc1c(cnn1C)-c1ccc3[nH]nc(c3c1)/C=C/2] | 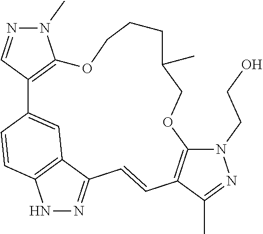 [SMILES: Cc1nn(CCO)c2c1/C=C/c1n[nH]c3ccc(cc13)-c1cnn(C)c1OCCCC(C)CO2] |
| B-2-5 | C-6-8 | D-1-5 | Ex. 26 |
| 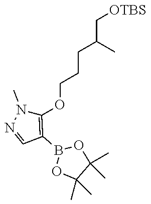 [SMILES: CC(CCCOc1c(B2OC(C)(C)C(C)(C)O2)cnn1C)CO[Si](C)(C)C(C)(C)C] | 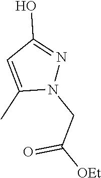 | 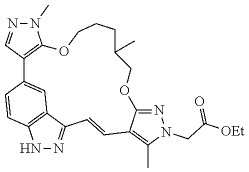 [SMILES: CCOC(=O)Cn1nc2c(c1C)/C=C/c1n[nH]c3ccc(cc13)-c1cnn(C)c1OCCCC(C)CO2] | 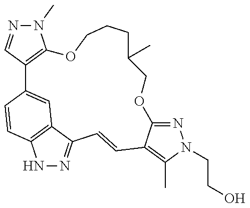 [SMILES: Cc1c2c(nn1CCO)OCC(C)CCCOc1c(cnn1C)-c1ccc3[nH]nc(c3c1)/C=C/2] |
| B-2-6 | C-6-6 | D-1-6 | Ex. 27 |
| 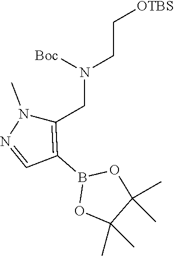 [SMILES: Cn1ncc(B2OC(C)(C)C(C)(C)O2)c1CN(CCO[Si](C)(C)C(C)(C)C)C(=O)OC(C)(C)C] | 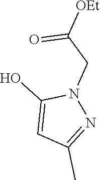 | 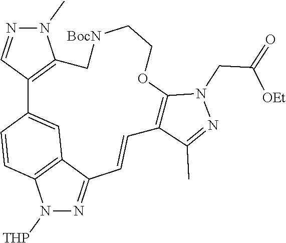 | 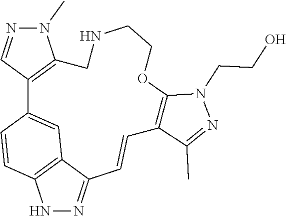 [SMILES: Cc1nn(CCO)c2c1/C=C/c1n[nH]c3ccc(cc13)-c1cnn(C)c1CNCCO2] |

### General Method E: Preparation of 1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-2-methylpropan-2-ol (Ex. 28) and 1-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]propan-2-one (Ex. 29)

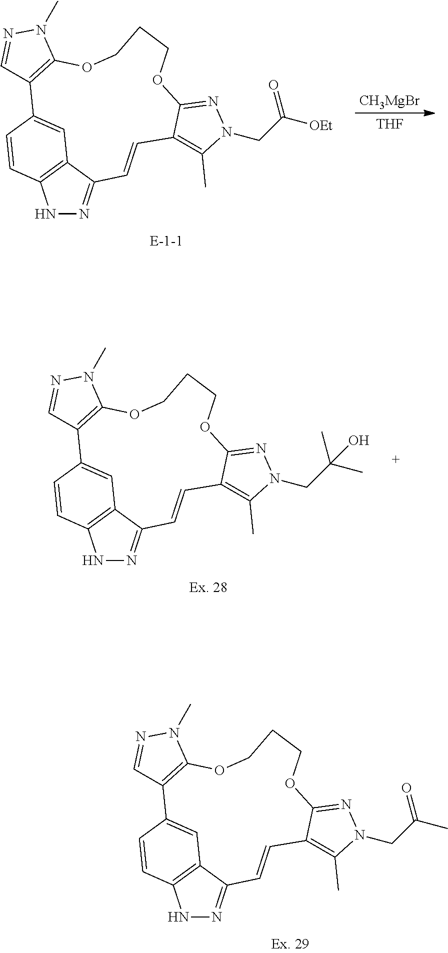

E-1-1 was prepared using General Method C.

To a solution of E-1-1 (100 mg, 0.216 mmol, 1 eq) in THF (2 mL) was added MeMgBr (3 M, 1.44 mL, 20 eq) at 0° C. The mixture was stirred at 20° C. for 2 h. On completion. The reaction mixture was quenched by addition NH₄Cl 10 mL at 0° C., and then diluted with H₂O (50 mL) and extracted with EA 150 mL (50 mL*3). The combined organic layers were dried over Na₂SO₄, filtered and concentrated under reduced pressure. The residue was purified by prep-HPLC to give Ex. 28 (4.62 mg, 0.0103 mmol, 4.76% yield) as yellow solid, and Ex. 29 (2.12 mg, 0.0049 mmol, 2.27% yield) as yellow solid.

Ex. 30 and Ex. 31 were prepared with General Method E.

### General Method F: Preparation of (17E)-8,16-dimethyl-14-[2-(pyrrolidin-1-yl)ethyl]-2,11,12,14-tetrahydro-8H,10H-3,5-etheno[1,5]dioxacyclopentadecino[10,11-c:15,14-c′:6,7-c″ ]tripyrazole (Ex. 32)

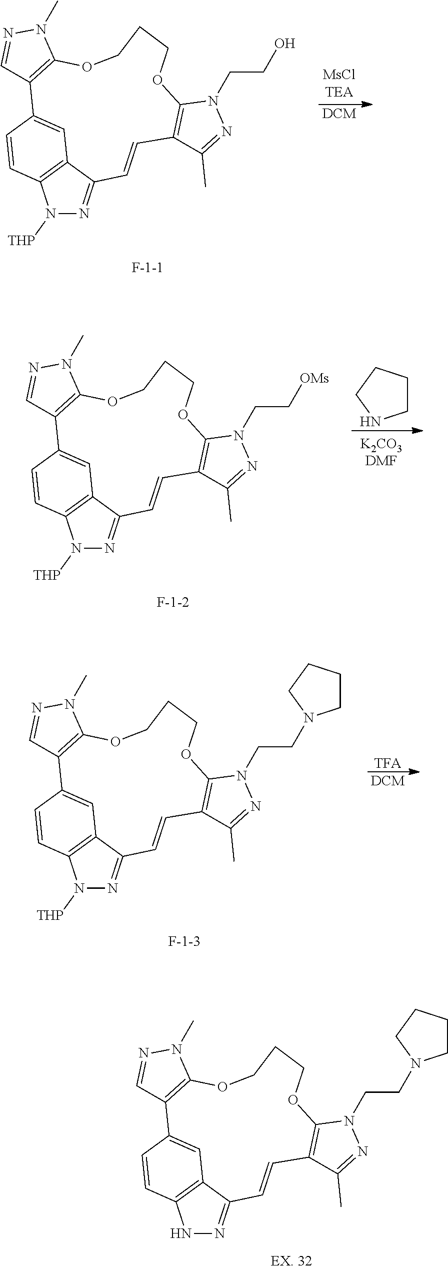

F-1-1 was prepared using General Method D.

Step 1. To a solution of F-1-1 (100 mg, 0.198 mmol, 1 eq) in DCM (1 mL) was added TEA (100 mg, 0.991 mmol, 5 eq) and MsCl (68.1 mg, 0.595 mmol, 3 eq) at 0° C. The mixture was stirred at 20° C. for 1 h. On completion, the reaction mixture was quenched by addition of sat. aqueous NaHCO₃ (5 mL) at 0° C., and then diluted with H₂O (20 mL) and extracted with DCM (20 mL*3). The combined organic layers were dried over Na₂SO₄, filtered and concentrated under reduced pressure to give F-2-1. LCMS: m/z 583.0 (M+1).

Step 2. To a solution of F-2-1 (110 mg, 0.189 mmol, 1 eq) in DMF (1 mL) was added K₂CO₃ (78.3 mg, 0.566 mmol, 3 eq) and pyrrolidine (26.9 mg, 0378 mmol, 2 eq). The mixture was stirred at 80° C. for 2 h. On completion, the reaction mixture was diluted with H₂O (30 mL) and extracted with EA (30 mL*3). The combined organic layers were washed with H₂O (30 mL*3), dried over Na₂SO₄, filtered and concentrated under reduced pressure. The residue was purified by column chromatography to give F-3-1 (37.0 mg, 0.0664 mmol, 35.1% yield) as colorless oil. LCMS: m/z 558.4 (M+1).

Step 4. To a solution of F-3-1 (37.0 mg, 0.0664 mmol, 1 eq) in DCM (1 mL) was added TFA (1 mL). The mixture was stirred at 20° C. for 1 h. On completion, the reaction mixture was concentrated under reduced pressure. The residue was purified by prep-HPLC to give Ex. 32 as TFA salt (4.12 mg, 0.0068 mmol, 10.2% yield) as brown solid.

Ex. 33 and Ex. 34 were prepared using General Method F.

### Preparation of triisopropyl-[2-[1-tetrahydropyran-2-yl-5-(4,4,5,5-tetramethyl-1,3,2-dioxaborolan-2-yl) indazol-3-yl]ethynyl]silane (G-1-1)

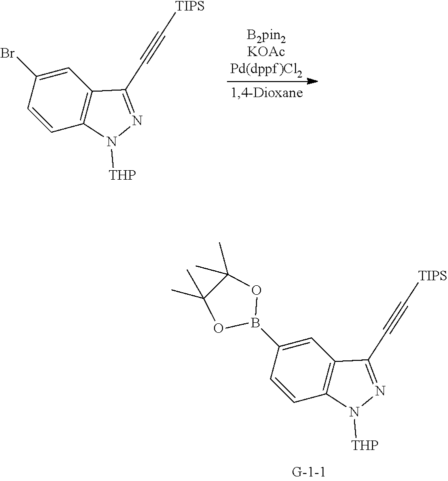

To a solution of 2-(5-bromo-1-tetrahydropyran-2-yl-indazol-3-yl)ethynyl-triisopropyl-silane (2 g, 4.33 mmol) in 1,4-Dioxane (20.86 mL) was added 4,4,5,5-tetramethyl-2-(4,4,5,5-tetramethyl-1,3,2-dioxaborolan-2-yl)-1,3,2-dioxaborolane (1.43 g, 5.63 mmol) and Potassium Acetate (1.28 g, 13.00 mmol). The mixture was stirred as argon was bubbled through for 5 minutes, followed by addition of catalyst, Pd(dppf)Cl₂ (158.55 mg, 0.217 mmol). The vessel sealed and heat to 85° C. for 18 hr. The reaction was diluted with DCM (80 mL) and water (80 mL) and the layers were separated. The aqueous layer was extracted again with DCM (2×40 mL). The combined organic layer was washed with brine and dried over sodium sulfate. Flash column chromatography (0-5% EA in Hexanes) provided triisopropyl-[2-[1-tetrahydropyran-2-yl-5-(4,4,5,5-tetramethyl-1,3,2-dioxaborolan-2-yl) indazol-3-yl]ethynyl]silane (1.87 g, 3.68 mmol, 84.85% yield). LCMS: m/z 509.28 (M+1).

### Preparation of [(3S)-3-(4-bromo-2-methyl-pyrazol-3-yl)oxybutoxy]-tert-butyl-dimethyl-silane (G-2-1)

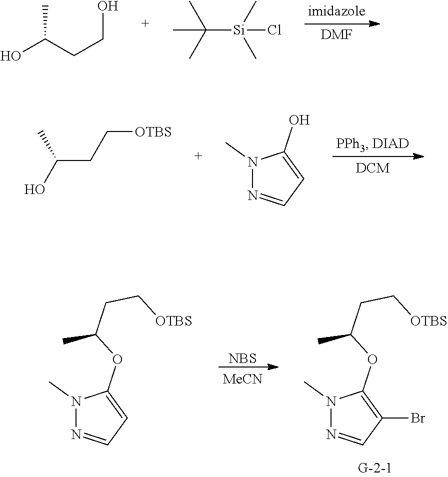

Step 1. To a solution of (3R)-butane-1,3-diol (2.03 g, 22.53 mmol, 2.02 mL) in DMF (42 mL) was added imidazole (1.84 g, 27.03 mmol) followed by addition of tert-butyl-chloro-dimethyl-silane (3.56 g, 23.65 mmol). The mixture was stirred at 0-22° C. for 4 hr. Reaction was diluted with DCM (150 mL) and water (150 mL) and the layers were separated. The aqueous layer was extracted again with DCM (2×50 mL). The combined organic layer was washed with 10% brine and dried over sodium sulfate. Flash column chromatography provided (2R)-4-[tert-butyl(dimethyl)silyl]oxybutan-2-ol (4.04 g, 19.77 mmol, 87.75% yield).

Step 2. To a solution of 2-methylpyrazol-3-ol (2.33 g, 23.72 mmol), (2R)-4-[tert-butyl(dimethyl)silyl]oxybutan-2-ol (4.04 g, 19.77 mmol) and triphenylphosphine (7.78 g, 29.65 mmol) in anhydrous DCM (13.5 mL) at 0° C. was added DIAD (6.40 g, 31.63 mmol, 6.2 mL). The reaction mixture is stirred for 2 hr at 0-22° C. Flash column chromatography provided tert-butyl-dimethyl-[(3S)-3-(2-methylpyrazol-3-yl)oxybutoxy]silane (3.2 g, 11.25 mmol, 56.91% yield).

Step 3. To a solution of tert-butyl-dimethyl-[(3S)-3-(2-methylpyrazol-3-yl)oxybutoxy]silane (5.62 g, 19.76 mmol) in acetonitrile (97 mL) was added NBS (3.52 g, 19.76 mmol). The reaction mixture was stirred at 0-22° C. for 3 hr and quenched with water (20 mL). Approximately 1/3 of acetonitrile was removed under reduced pressure. Remaining solution was worked up with DCM (75 mL) and water (75 mL). The aqueous layer was extracted twice more with DCM (2×30 mL). The combined organic layers were washed with brine and dried over sodium sulfate. Flash column chromatography provided [(3S)-3-(4-bromo-2-methyl-pyrazol-3-yl)oxybutoxy]-tert-butyl-dimethyl-silane (6.2 g, 17.06 mmol, 86.37% yield).

### Preparation of [(3R)-3-(4-bromo-2-methyl-pyrazol-3-yl)oxybutoxy]-tert-butyl-dimethyl-silane (G-2-2)

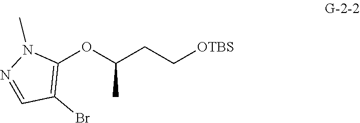 [SMILES: C[C@H](CCO[Si](C)(C)C(C)(C)C)Oc1c(Br)cnn1C]

G-2-2 was prepared following the same methods as G-2-1 using (3S)-butane-1,3-diol as starting material.

### General Method G: Preparation of (10S,17E)-8,10,14,16-tetramethyl-2,11,12,14-tetrahydro-8H,10H-3,5-etheno[1,5]dioxacyclopentadecino[10,11-c:15,14-c′:6,7-c″ ]tripyrazole (Ex. 35)

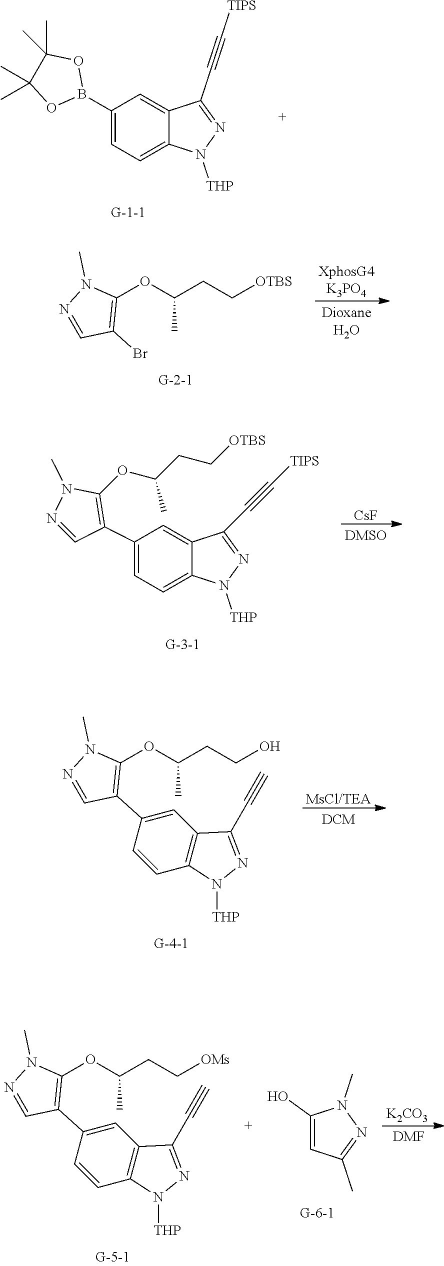

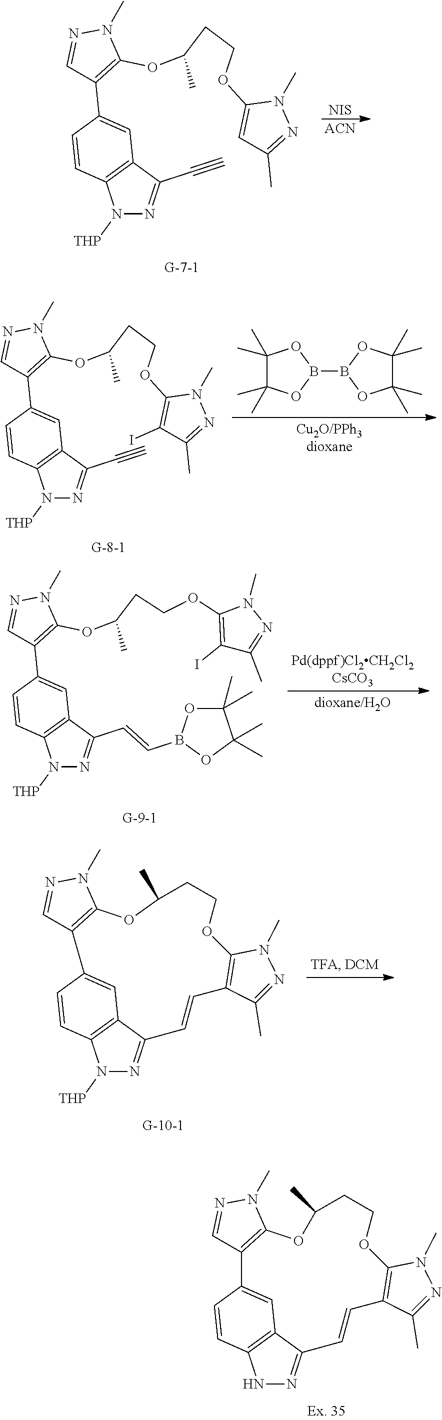

Step 1. To a solution of [(3S)-3-(4-bromo-2-methyl-pyrazol-3-yl)oxybutoxy]-tert-butyl-dimethyl-silane (600 mg, 1.65 mmol) in 1,4-dioxane (5.8 mL) was added triisopropyl-[2-[1-tetrahydropyran-2-yl-5-(4,4,5,5-tetramethyl-1,3,2-dioxaborolan-2-yl) indazol-3-yl]ethynyl]silane (834 mg, 1.64 mmol) and potassium phosphate tribasic (2 M in water, 2.46 mL). The reaction mixture was stirred as argon was bubbled through and the catalyst Xphos G4 (71 mg, 0.082 mmol) was added. The reaction vessel was sealed and heated to 80° C. for 18 hr. Flash column chromatography provided G-3-1 (561.15 mg, 0.843 mmol, 51.44% yield).

G-3-1 was converted to Ex. 35 using similar methods as Step 2-Step 8 in General Method C.

Ex. 35-Ex. 39 were prepared with General Method G using G-1-1, and the corresponding G-2-X, and C-6-X as shown below. C-6-X compounds are commercially available or made via conventional methods.

| G-2-X | C-6-X | Ex. # |
|---|---|---|
| G-2-1 | C-6-1 | Ex. 35 |
| 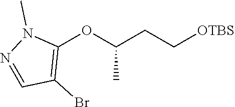 [SMILES: C[C@@H](CCO[Si](C)(C)C(C)(C)C)Oc1c(Br)cnn1C] | 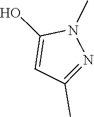 | 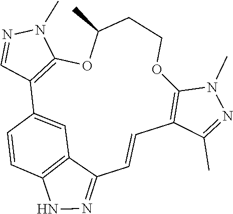 [SMILES: Cc1nn(C)c2c1/C=C/c1n[nH]c3ccc(cc13)-c1cnn(C)c1O[C@@H](C)CCO2] |
| G-2-2 | C-6-1 | Ex. 36 |
| 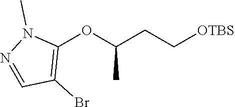 [SMILES: C[C@H](CCO[Si](C)(C)C(C)(C)C)Oc1c(Br)cnn1C] | 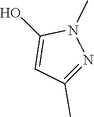 | 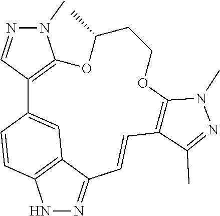 [SMILES: Cc1nn(C)c2c1/C=C/c1n[nH]c3ccc(cc13)-c1cnn(C)c1O[C@H](C)CCO2] |
| G-2-1 | C-6-3 | Ex. 37 |
| 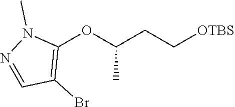 [SMILES: C[C@@H](CCO[Si](C)(C)C(C)(C)C)Oc1c(Br)cnn1C] | 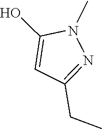 | 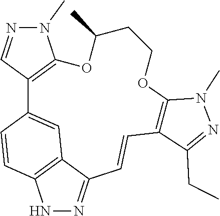 [SMILES: CCc1nn(C)c2c1/C=C/c1n[nH]c3ccc(cc13)-c1cnn(C)c1O[C@@H](C)CCO2] |
| G-2-1 | C-6-9 | Ex. 38 |
| 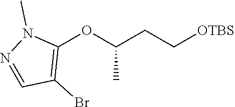 [SMILES: C[C@@H](CCO[Si](C)(C)C(C)(C)C)Oc1c(Br)cnn1C] | 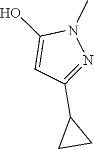 | 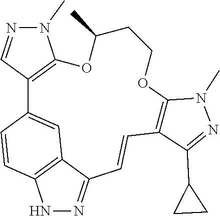 [SMILES: C[C@H]1CCOc2c(c(C3CC3)nn2C)/C=C/c2n[nH]c3ccc(cc23)-c2cnn(C)c2O1] |
| G-2-1 | C-6-10 | Ex. 39 |
| 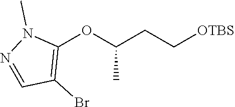 [SMILES: C[C@@H](CCO[Si](C)(C)C(C)(C)C)Oc1c(Br)cnn1C] | 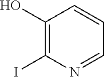 | 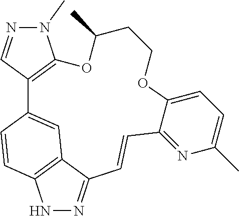 [SMILES: Cc1ccc2c(n1)/C=C/c1n[nH]c3ccc(cc13)-c1cnn(C)c1O[C@@H](C)CCO2] |

### Preparation of tert-butyl N-[2-[2-methyl-4-(4,4,5,5-tetramethyl-1,3,2-dioxaborolan-2-yl)pyrazol-3-yl]oxyethyl]carbamate (H-2-1)

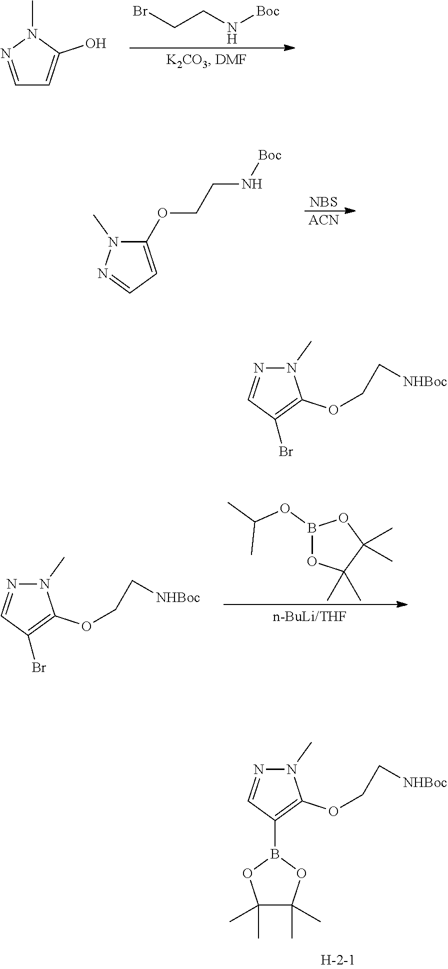

Step 1. To a mixture of 2-methylpyrazol-3-ol (5 g, 50.9 mmol, 1 eq) and tert-butyl N-(2-bromoethyl)carbamate (13.7 g, 61.1 mmol, 1.2 eq) in DMF (50 mL) was added K₂CO₃ (21.1 g, 152 mmol, 3 eq). The mixture was stirred at 80° C. for 2 hours. On completion, the mixture was quenched with water (100 mL) and extracted with ethyl acetate (40 mL×3). The combined organic phase was dried over anhydrous sodium sulfate, filtered and concentrated to give tert-butyl N-[2-(2-methylpyrazol-3-yl) oxyethyl]carbamate (11 g, 45.5 mmol, 89.45% yield) as black oil. LCMS: m/z 242.2 (M+1).

Step 2. To a solution of tert-butyl-N-[2-(2-methylpyrazol-3-yl)oxyethyl]carbamate (2 g, 7.8 mmol, 1 eq) in ACN (20 mL) was added NBS (1.44 g, 8.1 mmol, 1.03 eq). The mixture was stirred at 25° C. for 2 hr. On completion, the mixture was concentrated. The residue was purified by column chromatography to give tert-butyl N-[2-(4-bromo-2-methyl- pyrazol-3-yl)oxyethyl]carbamate (1.73 g, 66% yield) as red oil.

Step 3. To a mixture of tert-butyl N-[2-(4-bromo-2-methyl-pyrazol-3-yl)oxyethyl]carbamate (5.20 g, 16.2 mmol, 1 eq) in THF (70 mL) was added n-BuLi (2.5 M, 16.9 mL, 2.6 eq) at −78° C. The mixture was stirred at −78° C. for 1 hour under N₂ followed by addition of 2-isopropoxy-4,4,5,5-tetramethyl-1,3,2-dioxaborolane (6.04 g, 32.5 mmol, 2 eq). The mixture was stirred at −78° C. for 1 hour under N₂. On completion, the reaction mixture was quenched by addition MeOH (10 mL) and concentrated in vacuum to afford tert-butyl N-[2-[2-methyl-4-(4,4,5,5-tetramethyl-1,3,2-dioxaborolan-2-yl)pyrazol-3-yl]oxyethyl]carbamate (6 g, crude) as a yellow solid. LCMS: m/z 368.0 (M+1).

### Preparation of tert-butyl N-[2-[1-methyl-4-(4,4,5,5-tetramethyl-1,3,2-dioxaborolan-2-yl)pyrazol-3-yl]oxyethyl]carbamate (H-2-2)

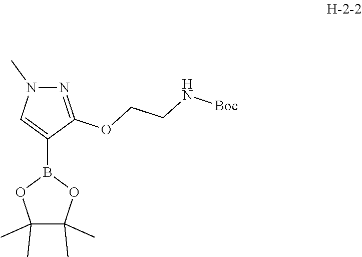 [SMILES: Cn1cc(B2OC(C)(C)C(C)(C)O2)c(OCCNC(=O)OC(C)(C)C)n1]

H-2-2 was prepared following similar methods as H-2-1.

### Preparation of tert-butyl N-methyl-N-[3-[2-methyl-4-(4,4,5,5-tetramethyl-1,3,2-dioxaborolan-2-yl)pyrazol-3-yl]oxyethyl]carbamate (H-2-3)

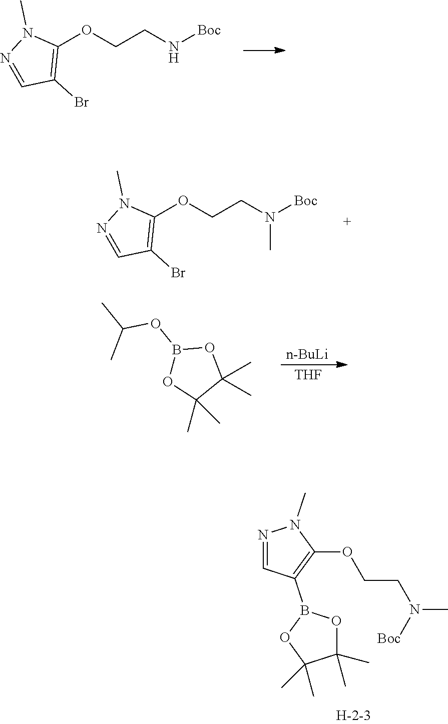

Step 1. To a solution of tert-butyl N-[2-(4-bromo-2-methyl-pyrazol-3-yl)oxyethyl]carbamate (20.0 g, 62.5 mmol, 1 eq) in DMF (120 mL) was added NaH (7.50 g, 187 mmol, 60% purity, 3 eq) at 0° C. and stirred for 30 mins. Then MeI (13.30 g, 93.7 mmol, 1.5 eq) was added dropwise at 0° C. The mixture was stirred at 25° C. for 1.5 hr. On completion, the mixture was quenched with water (20 mL) and extracted with ethyl acetate (25 mL×3). The combined organic phase was dried over anhydrous sodium sulfate, filtered and concentrated. The residue was purified by column chromatography to give tert-butyl N-[2-(4-bromo-2-methyl-pyrazol-3-yl)oxyethyl]-N-methyl-carbamate (20.0 g, 59.8 mmol, 95% yield) as a white oil. LCMS: m/z 336.7 (M+1).

Step 2. To a solution of tert-butyl N-[2-(4-bromo-2-methyl-pyrazol-3-yl)oxyethyl]-N-methyl-carbamate (20.0 g, 59.8 mmol, 1 eq) in THF (200 mL) was added n-BuLi (2.5 M, 23.9 mL, 1 eq) at −78° C., the mixture was stirred at this temperature for 30 mins followed by addition of 2-isopropoxy-4,4,5,5-tetramethyl-1,3,2-dioxaborolane (22.2 g, 119 mmol, 2 eq) dropwise at −78° C. The mixture was stirred at −78° C. for 2 hr. On completion, the mixture was quenched with water (200 mL) and extracted with ethyl acetate (250 mL×3). The combined organic phase was dried over anhydrous sodium sulfate, filtered and concentrated to give a residue. The residue was purified by column chromatography to give H-2-3 (21.2 g, 55.6 mmol, 92% yield) as a yellow oil. LCMS: m/z 381.9 (M+1).

### Preparation of tert-butyl N-methyl-N-[3-[1-methyl-4-(4,4,5,5-tetramethyl-1,3,2-dioxaborolan-2-yl)pyrazol-3-yl]oxyethyl]carbamate (H-2-4) and tert-butyl N-methyl-N-[3-[2,5-dimethyl-4-(4,4,5,5-tetramethyl-1,3,2-dioxaborolan-2-yl)pyrazol-3-yl]oxyethyl]carbamate (H-2-5)

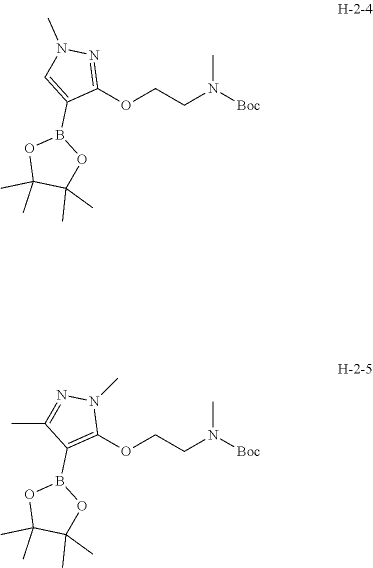 [SMILES: CN(CCOc1nn(C)cc1B1OC(C)(C)C(C)(C)O1)C(=O)OC(C)(C)C ; Cc1nn(C)c(OCCN(C)C(=O)OC(C)(C)C)c1B1OC(C)(C)C(C)(C)O1]

H-2-4 and H-2-5 were prepared with similar methods as H-2-3.

### Preparation of 3-(bromomethyl)-4-iodo-2,5-dimethyl-pyrazole (H-4-1) and 3-(bromomethyl)-4-iodo-1,5-dimethyl-pyrazole (H-4-2)

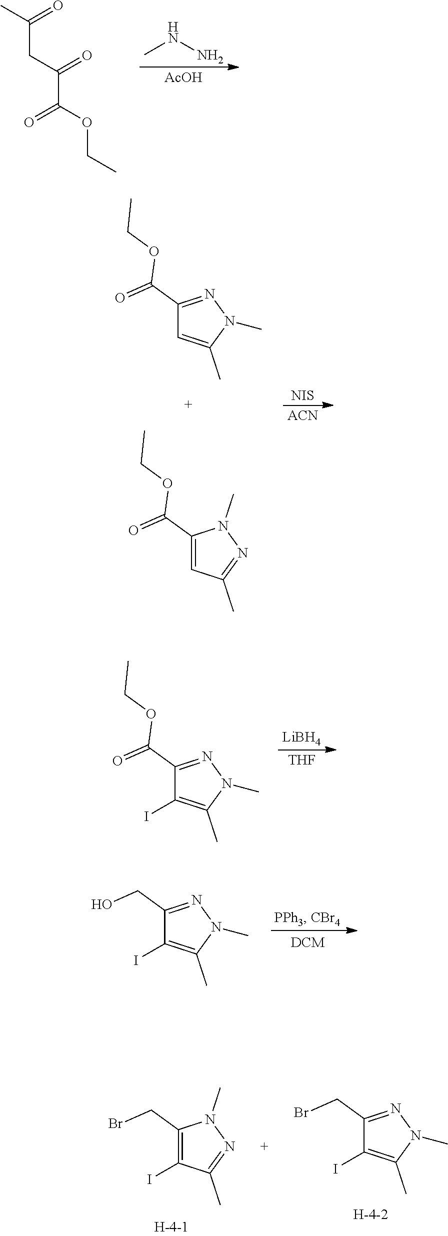

Step 1. To a solution of ethyl 2, 4-dioxohexanoate (10.0 g, 58.1 mmol, 1 eq) in AcOH (65.7 g, 1.09 mol, 18.8 eq) was added methylhydrazine (7.45 g, 64.7 mmol, 40% purity, 1.11 eq) at 0° C. The mixture was stirred at 15° C. for 5 hours and concentrated in vacuum. The residue was purified by combi flash chromatography (120 g silica gel column, EtOAc in PE from 0% to 50%) to provide ethyl 5-ethyl-1-methyl-pyrazole-3-carboxylate (10.1 g, 55.5 mmol, 95.5% yield) as yellow oil. ¹H NMR (400 MHz, CDCl₃) δ=6.59 (s, 1H), 4.39 (q, J=14.4, 7.2 Hz, 2H), 3.85 (s, 3H), 2.62 (q, J=14.4, 7.2 Hz, 2H), 1.39 (t, J=7.2 Hz, 3H), 1.28 (t, J=7.6 Hz, 3H).

Ethyl 5-ethyl-2-methyl-pyrazole-3-carboxylate (1.33 g, 7.30 mmol, 12.6% yield) was obtained as colorless oil. ¹H NMR (400 MHz, CDCl₃) δ=6.65 (s, 1H), 4.34 (q, J=7.2 Hz, 2H), 4.18-4.11 (m, 4H), 2.65 (q, J=15.2, 7.6 Hz, 2H), 1.38 (t, J=7.2 Hz, 3H), 1.25 (t, J=7.6 Hz, 3H).

Step 2. To a solution of ethyl 1,5-dimethylpyrazole-3-carboxylate (3.00 g, 17.8 mmol, 1 eq) in MeCN (30 mL) was added NIS (4.41 g, 19.6 mmol, 1.1 eq) at 0° C. The mixture was stirred at 25° C. for 3 hours. The residue was poured into ice-water (200 mL). The aqueous phase was extracted with ethyl acetate (100 mL×3). The combined organic phase was washed with brine (100 mL), dried with anhydrous Na₂SO₄, filtered and concentrated in vacuum. The residue was purified by column chromatography to give ethyl 4-iodo-1,5-dimethyl-pyrazole-3-carboxylate (4.20 g, 14.3 mmol, 80.1% yield) as a white solid. ¹H NMR (400 MHz, CDCl₃) δ=4.41 (q, J=7.2 Hz, 2H), 3.93 (s, 3H), 2.35 (s, 3H), 1.41 (t, J=7.2 Hz, 3H).

Step 3. To a solution of ethyl 4-iodo-1,5-dimethyl-pyrazole-3-carboxylate (2.00 g, 6.80 mmol, 1 eq) in THF (30 mL) was added LiBH₄ (296 mg, 13.6 mmol, 2 eq) in three portions at 25° C. The mixture was stirred at 25° C. for 1 hour. To the mixture was added aq. NH₄Cl (10 mL) dropwise at 0° C. under N₂. The mixture was stirred for 30 min. On completion, the aqueous phase was extracted with ethyl acetate (30 mL*2). The combined organic phase was washed with brine (20 mL), dried with anhydrous Na₂SO₄, filtered and concentrated in vacuum to give (4-iodo-1,5-dimethyl-pyrazol-3-yl)methanol (1.4 g, 81% yield) as a white solid. LCMS: 252.8 (M+1).

Step 4. To a mixture of (4-iodo-1,5-dimethyl-pyrazol-3-yl)methanol (300 mg, 1.19 mmol, 1 eq) in DCM (10 mL) was added PPh₃ (468 mg, 1.79 mmol, 1.5 eq) at 0° C. Then to the mixture was added CBr₄ (592 mg, 1.79 mmol, 1.5 eq) at 0° C. The mixture was stirred at 25° C. for 1 hour. On completion, the mixture was concentrated in vacuum. The residue was purified by column chromatography to give 3-(bromomethyl)-4-iodo-1,5-dimethyl-pyrazole (H-4-2, 260 mg, 69% yield, 100% purity) as a white solid. LCMS m/z 316.7 (M+1); ¹H NMR (400 MHz, CDCl₃) δ=4.44 (s, 2H), 3.84 (s, 3H), 2.29 (s, 3H).

Ethyl 5-ethyl-2-methyl-pyrazole-3-carboxylate was converted to 3-(bromomethyl)-4-iodo-2,5-dimethyl-pyrazole (H-4-1) following similar methods.

### Preparation of 5-(bromomethyl)-3-ethyl-4-iodo-1-methyl-1H-pyrazole (H-4-3)

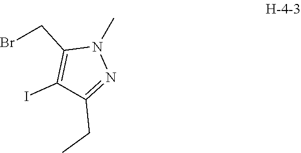

H-4-3 was prepared following similar methods as H-4-1.

### Preparation of 2-[3-(bromomethyl)-4-iodo-5-methyl-pyrazol-1-yl]ethoxy-tert-butyl-dimethyl-silane (H-4-4) and 2-[5-(bromomethyl)-4-iodo-3-methyl-pyrazol-1-yl]ethoxy-tert-butyl-dimethyl-silane (H-4-5)

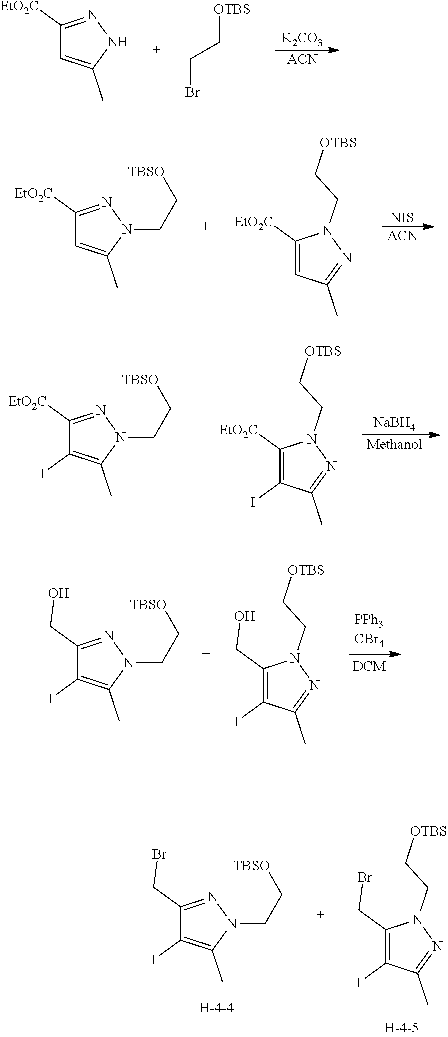

Step 1. A mixture of ethyl 5-methyl-1H-pyrazole-3-carboxylate (1 g, 6.49 mmol), 2-bromoethoxy-tert-butyl-dimethyl-silane (3.10 g, 12.97 mmol, 2.78 mL), potassium carbonate, anhydrous (2.69 g, 19.46 mmol) in acetonitrile (28.5 mL) was stirred at room temperature overnight. The mixture was then diluted with water (15 mL) and extracted with ethyl acetate (3×75 mL). The combined organic layers were washed with brine and dried over anhydrous sodium sulfate. The solvent was evaporated, and the crude residue was purified by flash column chromatography to furnish a mixture of ethyl 1-[2-[tert-butyl(dimethyl)silyl]oxyethyl]-5-methyl-pyrazole-3-carboxylate and ethyl 2-[2-[tert-butyl(dimethyl)silyl]oxyethyl]-5-methyl-pyrazole-3-carboxylate (1.57 g, 5.01 mmol, 77.31% yield). LCMS: m/z 313.03 (M+1)

Step 2. A mixture of ethyl 1-[2-[tert-butyl(dimethyl)silyl]oxyethyl]-5-methyl-pyrazole-3-carboxylate and ethyl 2-[2-[tert-butyl(dimethyl)silyl]oxyethyl]-5-methyl-pyrazole-3-carboxylate (785.00 mg, 2.51 mmol) was dissolved in acetonitrile (35 mL). N-Iodosuccinimide (2.26 g, 10.05 mmol) was added to this solution, and the mixture was heated to 60° C. for 12 h. The solvent was evaporated under reduced pressure and the residue was purified by flash column chromatography to obtain a mixture of ethyl 1-[2-[tert-butyl(dimethyl)silyl]oxyethyl]-4-iodo-5-methyl-pyrazole-3-carboxylate and ethyl 2-[2-[tert-butyl(dimethyl)silyl]oxyethyl]-4-iodo-5-methyl-pyrazole-3-carboxylate (0.97 g, 2.21 mmol, 88.08% yield). LCMS: m/z 438.82 (M+H).

Step 3. To a mixture of ethyl 1-[2-[tert-butyl(dimethyl)silyl]oxyethyl]-4-iodo-5-methyl-pyrazole-3-carboxylate and ethyl 2-[2-[tert-butyl(dimethyl)silyl]oxyethyl]-4-iodo-5-methyl-pyrazole-3-carboxylate (0.97 g, 2.21 mmol) in methanol (11.65 mL) was added sodium borohydride (178.86 mg, 4.73 mmol) and the mixture was stirred overnight at room temperature. The reaction was then diluted with water (5 mL) and ethyl acetate (50 mL). The layers were separated, and the aqueous phase was extracted with ethyl acetate (2×25 mL). The combined organic layers were dried over anhydrous sodium sulfate, filtered, dried and purified by flash column chromatography to afford a mixture of [4-iodo-5-methyl-1-(2-trimethylsilylethoxymethyl)pyrazol-3-yl]methanol and [4-iodo-5-methyl-2-(2-trimethylsilylethoxymethyl)pyrazol-3-yl]methanol (745 mg, 2.02 mmol, 73.13% yield). LCMS: m/z 396.87 (M+H).

Step 4. To a mixture of [1-[2-[tert-butyl(dimethyl)silyl]oxyethyl]-4-iodo-5-methyl-pyrazol-3-yl]methanol and [2-[2-[tert-butyl(dimethyl)silyl]oxyethyl]-4-iodo-5-methyl-pyrazol-3-yl]methanol (745 mg, 1.88 mmol) in DCM (15.41 mL) was added Triphenylphosphine (739.54 mg, 2.82 mmol) and Carbon tetrabromide (935.05 mg, 2.82 mmol, 273.41 L) at 0° C., and the mixture was stirred at room temperature for 4 hr. The reaction was quenched with water (10 mL) and extracted with DCM (3×50 mL). The combined organic layers were dried over anhydrous sodium sulfate, filtered, dried and purified by flash column chromatography to afford 2-[3-(bromomethyl)-4-iodo-5-methyl-pyrazol-1-yl]ethoxy-tert-butyl-dimethyl-silane (H-4-4, 192 mg, 0.418 mmol, 22.24% yield) and 2-[5-(bromomethyl)-4-iodo-3-methyl-pyrazol-1-yl]ethoxy-tert-butyl-dimethyl-silane (H-4-5, 564 mg, 1.23 mmol, 65.34% yield). LCMS: [M+H]⁺ m/z=458.73

### General Method H: (17E)-8,14,16-trimethyl-2,10,11,12,13,14-hexahydro-8H-3,5-ethenotripyrazolo[3,4-f:3′,4′-j:4″,3″-n][1,4]oxazacyclopentadecine (Ex. 40)

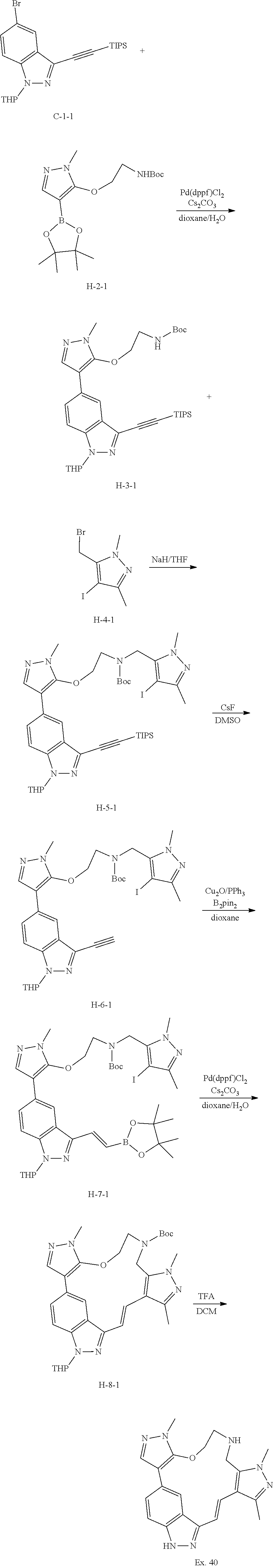

Step 1. To a mixture of 2-(5-bromo-1-tetrahydropyran-2-yl-indazol-3-yl)ethynyl-triisopropyl-silane (5.18 g, 11.2 mmol, 0.75 eq) and tert-butyl N-[2-[2-methyl-4-(4,4,5,5-tetramethyl-1,3,2-dioxaborolan-2-yl)pyrazol-3-yl]oxyethyl]carbamate (5.50 g, 15.0 mmol, 1 eq) in dioxane (80 mL) and H₂O (16 mL) were added Cs₂CO₃ (14.6 g, 44.9 mmol, 3 eq) and Pd(dppf)Cl₂ (1.10 g, 1.50 mmol, 0.1 eq). The mixture was stirred at 80° C. for 12 hours. On completion, the residue was diluted with H₂O (10 mL) and extracted with EtOAc (10 mL*3). The combined organic layers were washed with brine (10 mL*3), dried over Na₂SO₄, filtered and concentrated under reduced pressure. The residue was purified by flash silica gel chromatography followed by reversed-phase HPLC to afford tert-butyl N-[2-[2-methyl-4-[1-tetrahydropyran-2-yl-3-(2-triisopropylsilylethynyl) indazol-5-yl]pyrazol-3-yl]oxyethyl]carbamate (3.70 g, 39% yield, 100% purity) as a yellow oil. ¹H NMR (400 MHz, DMSO-d₆) δ=7.82 (d, J=8.4 Hz, 1H), 7.75-7.70 (m, 2H), 7.02 (br s, 1H), 5.88 (d, J=9.2 Hz, 1H), 3.92 (s, 2H), 3.90 (s, 1H), 3.79-3.73 (m, 1H), 3.70 (s, 3H), 3.24 (d, J=5.6 Hz, 2H), 2.38-2.29 (m, 1H), 1.99 (s, 2H), 1.78-1.68 (m, 1H), 1.59 (s, 2H), 1.35 (s, 9H), 1.18-1.14 (m, 18H), 1.14-1.12 (m, 3H).

Step 2. To a mixture of tert-butyl N-[2-[2-methyl-4-[1-tetrahydropyran-2-yl-3-(2-triisopropylsilylethynyl) indazol-5-yl]pyrazol-3-yl]oxyethyl]carbamate (2.00 g, 3.22 mmol, 1 eq) in THE (35 mL) was added NaH (386 mg, 9.65 mmol, 60.0% purity, 3 eq) in portions at 0° C. The mixture was stirred at 0° C. for 1 hour followed by addition of 5-(bromomethyl)-4-iodo-1,3-dimethyl-pyrazole (1.22 g, 3.86 mmol, 1.2 eq). The mixture was stirred at 60° C. for 12 hours. On completion, the residue was diluted with H₂O (10 mL) and extracted with EtOA (10 mL*3). The combined organic layers were washed with brine (10 mL*3), dried over Na₂SO₄, filtered and concentrated under reduced pressure. The residue was purified by flash silica gel chromatography to afford tert-butyl N-[(4-iodo-2,5-dimethyl-pyrazol-3-yl)methyl]-N-[2-[2-methyl-4-[1-tetrahydropyran-2-yl-3-(2-triisopropylsilylethynyl) indazol-5-yl]pyrazol-3-yl]oxyethyl]carbamate (2.90 g, crude) as a yellow solid. LCMS: m/z 856.5 (M+1).

Step 3. To a mixture of tert-butyl N-[(4-iodo-2,5-dimethyl-pyrazol-3-yl)methyl]-N-[2-[2-methyl-4-[1-tetrahydropyran-2-yl-3-(2-triisopropylsilylethynyl) indazol-5-yl]pyrazol-3-yl]oxyethyl]carbamate (2.00 g, 2.34 mmol, 1 eq) in DMSO (20 mL) was added CsF (710 mg, 4.67 mmol, 2 eq). The mixture was stirred at 40° C. for 12 hours. On completion, the residue was diluted with H₂O (10 mL) and extracted with EtOAc (10 mL * 3). The combined organic layers were washed with brine (10 mL*3), dried over Na₂SO₄, filtered and concentrated under reduced pressure. The residue was purified by flash silica gel chromatography to afford tert-butyl N-[2-[4-(3-ethynyl-1-tetrahydropyran-2-yl-indazol-5-yl)-2-methyl-pyrazol-3-yl]oxyethyl]-N-[(4-iodo-2,5-dimethyl-pyrazol-3-yl)methyl]carbamate (1.20 g, 68% yield, 93.2% purity) as a yellow solid. LCMS: m/z 700.4 (M+1).

Step 4. To a mixture of tert-butyl N-[2-[4-(3-ethynyl-1-tetrahydropyran-2-yl-indazol-5-yl)-2-methyl-pyrazol-3-yl]oxyethyl]-N-[(4-iodo-2,5-dimethyl-pyrazol-3-yl)methyl]carbamate (1.75 g, 2.50 mmol, 1 eq) and 4,4,5,5-tetramethyl-2-(4,4,5,5-tetramethyl-1,3,2-dioxaborolan-2-yl)-1,3,2-dioxaborolane (952 mg, 3.75 mmol, 1.5 eq) in dioxane (20 mL) were added Cu₂O (179 mg, 1.25 mmol, 0.5 eq) and PPh₃ (656 mg, 2.50 mmol, 1 eq) under N₂. The mixture was stirred at 100° C. for 12 hours. On completion, the residue was diluted with H₂O (10 mL) and extracted with EtOAc (10 mL*3). The combined organic layers were washed with brine (10 mL*3), dried over Na₂SO₄, filtered and concentrated under reduced pressure. The residue was purified by flash silica gel chromatography to afford tert-butyl N-[(4-iodo-2,5-dimethyl-pyrazol-3-yl)methyl]-N-[2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxyethyl]carbamate (1.90 g, 91% yield) as a yellow solid. ¹H NMR (400 MHz, CDCl₃) δ=8.04 (s, 1H), 7.78-7.71 (m, 1H), 7.70-7.64 (m, 1H), 7.59 (s, 1H), 7.57-7.55 (m, 1H), 6.52 (d, J=18.8 Hz, 1H), 5.74 (dd, J=2.8, 8.8 Hz, 1H), 4.62 (s, 2H), 4.04-3.85 (m, 4H), 3.80-3.68 (m, 5H), 3.38 (t, J=6.0 Hz, 2H), 2.64-2.49 (m, 1H), 2.27-2.15 (m, 5H), 1.84-1.65 (m, 4H), 1.42-1.30 (m, 21H).

Step 5. To a mixture of tert-butyl N-[(4-iodo-2,5-dimethyl-pyrazol-3-yl)methyl]-N-[2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxyethyl]carbamate (1.90 g, 2.30 mmol, 1 eq) in dioxane (20 mL) and H₂O (4 mL) were added Pd(dppf)Cl₂ (168 mg, 0.230 mmol, 0.1 eq) and Cs₂CO₃ (1.50 g, 4.59 mmol, 2 eq) under N₂, The mixture was stirred at 80° C. for 12 hours. On completion, the residue was diluted with H₂O (20 mL) and extracted with EtOAc (20 mL*3). The combined organic layers were washed with brine (10 mL*3), dried over Na₂SO₄, filtered and concentrated under reduced pressure. The residue was purified by flash silica gel chromatography to afford H-8-1 (600 mg, 45% yield) as a yellow solid. LCMS: 574.3 (M+1)

Step 6. To a mixture of H-8-1 (600 mg, 1.05 mmol, 1 eq) in DCM (6 mL) was added TFA (4.62 g, 40.5 mmol, 38.7 eq). The mixture was stirred at 25° C. for 1 hour. On completion, the reaction mixture was added NaHCO₃ until pH=7-8. After filtration, the filtrate was diluted with H₂O (5 mL) and extracted with EtOAc (5 mL*3). The combined organic layers were washed with brine (5 mL*3), dried over Na₂SO₄, filtered and concentrated under reduced pressure. The residue was purified by flash silica gel chromatography to afford Ex. 40.

Ex. 40-Ex. 42 were prepared with General Method H using corresponding C-1-1, and the corresponding H-2-X, and H-4-X as shown in the table. Ex. 43-Ex. 47 were prepared with General Method H using C-1-1, and the corresponding H-2-X, and H-4-X with an additional de-Boc step after the first step as shown below:

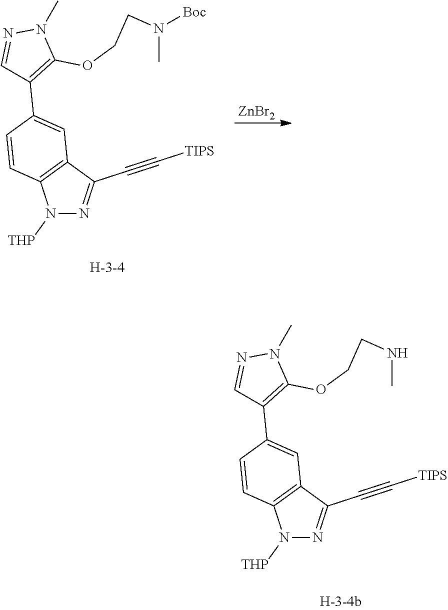

To a solution of tert-butyl N-methyl-N-[2-[2-methyl-4-[1-tetrahydropyran-2-yl-3-(2-triisopropylsilylethynyl) indazol-5-yl]pyrazol-3-yl]oxyethyl]carbamate (6.00 g, 9.44 mmol, 1 eq) in DCM (120 mL) was added ZnBr₂ (10.6 g, 47.1 mmol, 5 eq). The mixture was stirred at 25° C. for 12 hr. On completion, the mixture was concentrated to give N-methyl-2-[2-methyl-4-[1-tetrahydropyran-2-yl-3-(2-triisopropylsilylethynyl) indazol-5-yl]pyrazol-3-yl]oxy-ethanamine (4.00 g, 7.47 mmol, 79% yield, crude) as a brown oil. LCMS: m/z 536.2 (M+1)

| H-2-X | H-4-X | Ex. # |
|---|---|---|
| H-2-1 | H-4-1 | Ex. 40 |
| 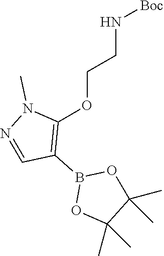 [SMILES: Cn1ncc(B2OC(C)(C)C(C)(C)O2)c1OCCNC(=O)OC(C)(C)C] | 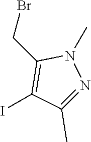 | 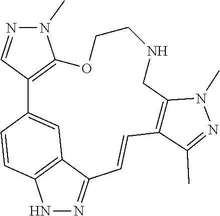 [SMILES: Cc1nn(C)c2c1/C=C/c1n[nH]c3ccc(cc13)-c1cnn(C)c1OCCNC2] |
| H-2-1 | H-4-2 | Ex. 41 |
| 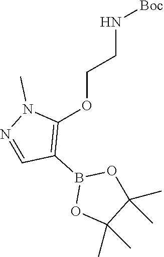 [SMILES: Cn1ncc(B2OC(C)(C)C(C)(C)O2)c1OCCNC(=O)OC(C)(C)C] | 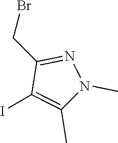 | 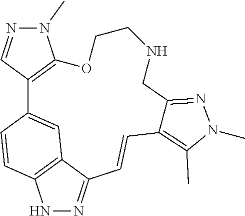 [SMILES: Cc1c2c(nn1C)CNCCOc1c(cnn1C)-c1ccc3[nH]nc(c3c1)/C=C/2] |
| H-2-2 | H-4-1 | Ex. 42 |
| 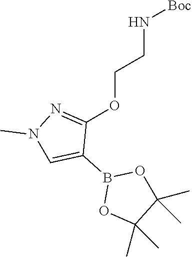 | 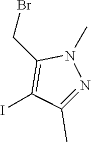 | 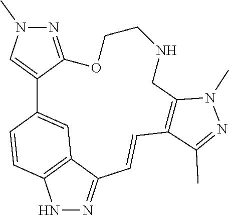 |
| H-2-3 | H-4-1 | Ex. 43 |
| 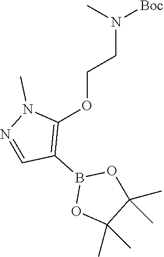 [SMILES: CN(CCOc1c(B2OC(C)(C)C(C)(C)O2)cnn1C)C(=O)OC(C)(C)C] | 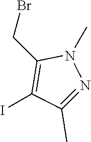 | 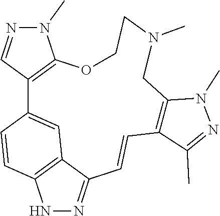 [SMILES: Cc1nn(C)c2c1/C=C/c1n[nH]c3ccc(cc13)-c1cnn(C)c1OCCN(C)C2] |
| H-2-3 | H-4-2 | Ex.44 |
| 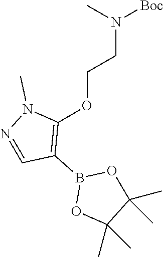 [SMILES: CN(CCOc1c(B2OC(C)(C)C(C)(C)O2)cnn1C)C(=O)OC(C)(C)C] | 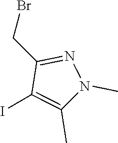 | 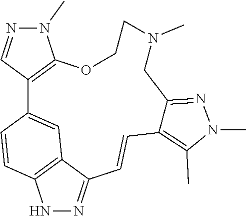 [SMILES: Cc1c2c(nn1C)CN(C)CCOc1c(cnn1C)-c1ccc3[nH]nc(c3c1)/C=C/2] |
| H-2-3 | H-4-3 | Ex. 45 |
| 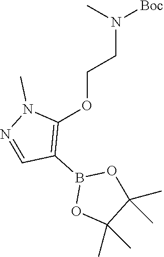 [SMILES: CN(CCOc1c(B2OC(C)(C)C(C)(C)O2)cnn1C)C(=O)OC(C)(C)C] | 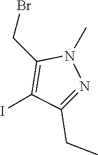 | 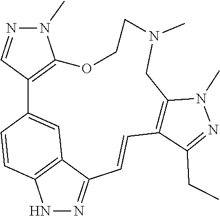 [SMILES: CCc1nn(C)c2c1/C=C/c1n[nH]c3ccc(cc13)-c1cnn(C)c1OCCN(C)C2] |
| H-2-4 | H-4-1 | Ex. 46 |
| 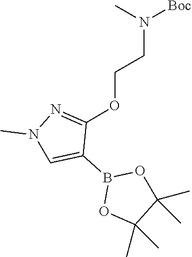 | 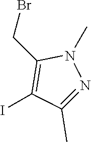 | 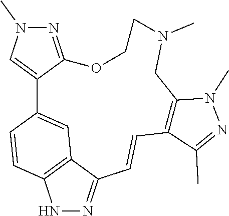 |
| H-2-5 | H-4-1 | Ex. 47 |
| 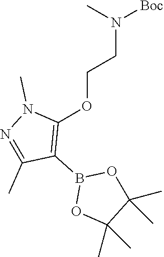 [SMILES: Cc1nn(C)c(OCCN(C)C(=O)OC(C)(C)C)c1B1OC(C)(C)C(C)(C)O1] | 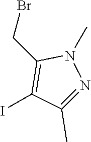 | 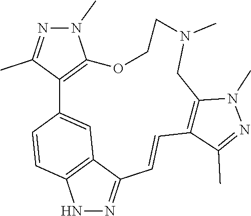 |

### Preparation of tert-butyl N-[(2S)-2-(4-bromo-2-methyl-pyrazol-3-yl)oxypropyl]carbamate (I-2-1)

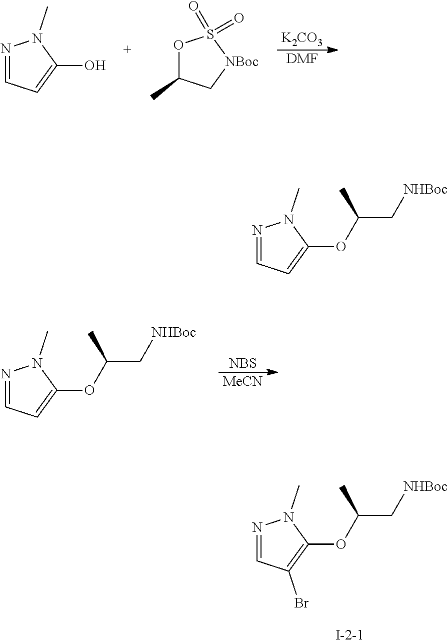

Step 1. A mixture of 2-methylpyrazol-3-ol (1.00 g, 10.2 mmol), tert-butyl (5R)-5-methyl-2,2-dioxo-oxathiazolidine-3-carboxylate (2.42 g, 10.2 mmol) in DMF (49 mL), and potassium carbonate, anhydrous (4.23 g, 30.6 mmol) was heated to 80° C. for 18 hr. The suspension was filtered over Celite and diluted with DCM (500 mL). A aqueous solution of citric acid (1 M, 100 mL) was added, and the mixture was stirred for 1 h until only product peak was observed by LCMS. The layers were separated, and the aqueous layer extracted with DCM (2×150 mL). The combined organic layers were washed with brine, dried over anhydrous sodium sulfate. The solvent was evaporated and the crude residue was purified by flash column chromatography to furnish tert-butyl N-[(2S)-2-(2-methylpyrazol-3-yl)oxypropyl]carbamate (2.59 g, 10.14 mmol, 99.5% yield). LCMS: m/z 255.99 (M+1).

Step 2. tert-butyl N-[(2S)-2-(2-methylpyrazol-3-yl)oxypropyl]carbamate (1.3 g, 5.09 mmol) was dissolved in acetonitrile (63 mL). N-Bromosuccinimide (1.04 g, 5.86 mmol) was added to this solution at 0° C. The mixture was stirred at room temperature for 3 hr. The solvent was evaporated under reduced pressure and the residue was purified by flash column chromatography to obtain tert-butyl N-[(2S)-2-(4-bromo-2-methyl-pyrazol-3-yl)oxypropyl]carbamate (1.23 g, 3.68 mmol, 72.3% yield). LCMS: m/z 335.78 (M+1).

### General Method I: (10S,17E)-8,10,15,16-tetramethyl-2,10,11,12,13,15-hexahydro-8H-3,5-ethenotripyrazolo[3,4-f:3′,4′-j:4″,3″-n][1,4]oxazacyclopentadecine (Ex. 48)

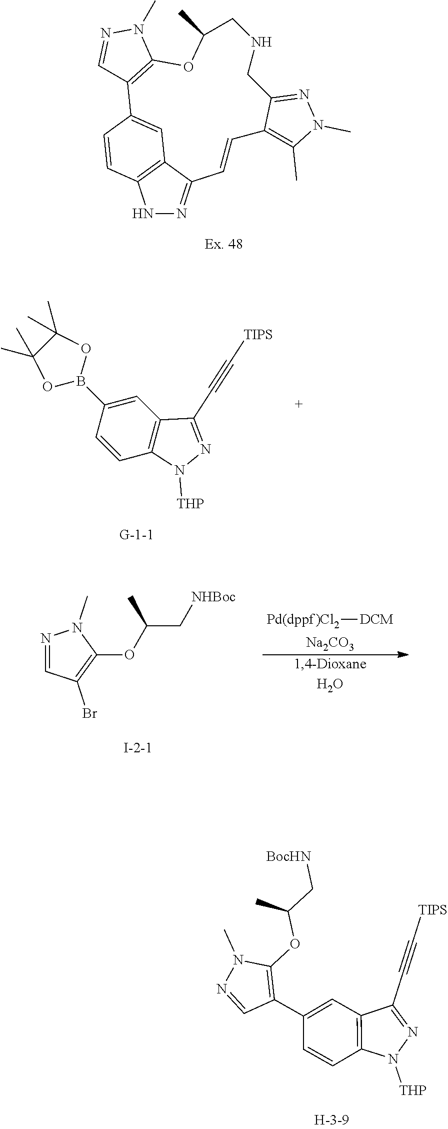

To tert-butyl N-[(2S)-2-(4-bromo-2-methyl-pyrazol-3-yl)oxypropyl]carbamate (564 mg, 1.69 mmol) and triisopropyl-[2-[1-tetrahydropyran-2-yl-5-(4,4,5,5-tetramethyl-1,3,2-dioxaborolan-2-yl) indazol-3-yl]ethynyl]silane (944.08 mg, 1.86 mmol) in 1,4-dioxane (5 mL) and water (5 mL) was added Sodium carbonate (536.60 mg, 5.06 mmol). The mixture was stirred for 5 minutes as argon was bubbled through, followed by addition of catalyst Pd(dppf)Cl₂-DCM (68.91 mg, 0.0844 mmol). The vessel was sealed and heat to 85° C. for 18 hr. The reaction was diluted with DCM (20 mL) and water (5 mL) and the layers were separated. The aqueous layer was extracted again with DCM (2×20 mL). The combined organic layers were washed with brine and dried over anhydrous magnesium sulfate. The solvent was evaporated and the residue was purified by flash column chromatography to obtain H-3-9 (657 mg, 1.03 mmol, 61.2% yield). LCMS: m/z 636.04 (M+1).

H-3-9 was converted to Ex. 48 following Step 2-Step 5 in General Method H.

Ex. 48-Ex. 50 were prepared with General Method I using G-1-1, and the corresponding I-2-X and H-4-X as shown in the Table.

| I-2-X | H-4-X | Ex. # |
|---|---|---|
| I-2-1 | H-4-2 | Ex. 48 |
| 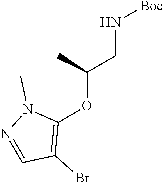 [SMILES: C[C@@H](CNC(=O)OC(C)(C)C)Oc1c(Br)cnn1C] | 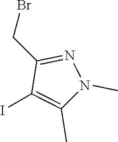 | 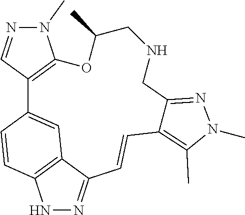 [SMILES: Cc1c2c(nn1C)CNC[C@H](C)Oc1c(cnn1C)-c1ccc3[nH]nc(c3c1)/C=C/2] |
| I-2-1 | H-4-4 | Ex. 49 |
| 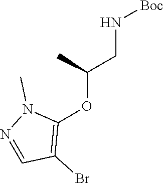 [SMILES: C[C@@H](CNC(=O)OC(C)(C)C)Oc1c(Br)cnn1C] | 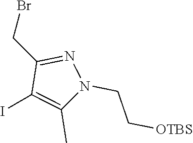 [SMILES: Cc1c(I)c(CBr)nn1CCO[Si](C)(C)C(C)(C)C] | 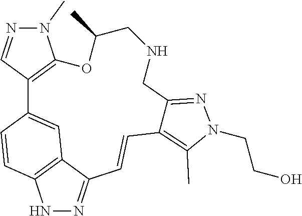 [SMILES: Cc1c2c(nn1CCO)CNC[C@H](C)Oc1c(cnn1C)-c1ccc3[nH]nc(c3c1)/C=C/2] |
| I-2-1 | H-4-5 | Ex. 50 |
| 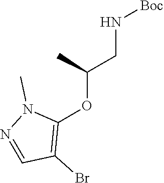 [SMILES: C[C@@H](CNC(=O)OC(C)(C)C)Oc1c(Br)cnn1C] | 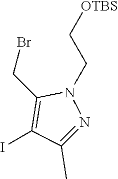 [SMILES: Cc1nn(CCO[Si](C)(C)C(C)(C)C)c(CBr)c1I] | 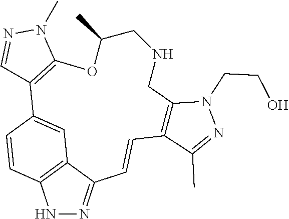 [SMILES: Cc1nn(CCO)c2c1/C=C/c1n[nH]c3ccc(cc13)-c1cnn(C)c1O[C@@H](C)CNC2] |

### General Method J: 1-[(17E)-8,14,16-trimethyl-2,8,10,11,13,14-hexahydro-12H-3,5-ethenotripyrazolo[3,4-f 3′,4′-j:4″,3″-n][1,4]oxazacyclopentadecin-12-yl]ethan-1-one (Ex. 51)

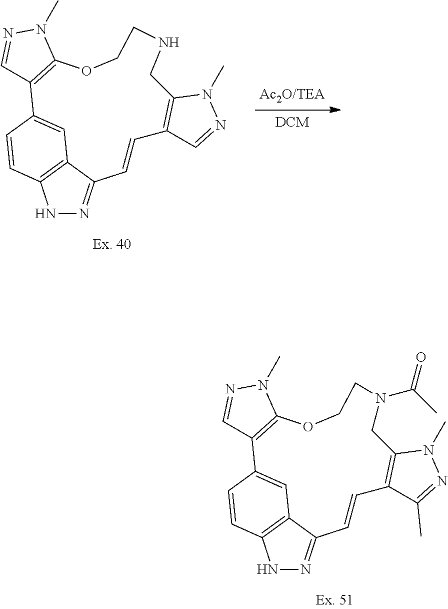 [SMILES: Cn1ncc2c1CNCCOc1c(cnn1C)-c1ccc3[nH]nc(c3c1)/C=C/2 ; CC(=O)N1CCOc2c(cnn2C)-c2ccc3[nH]nc(c3c2)/C=C/c2c(C)nn(C)c2C1]

To a mixture of Ex. 38 (70.0 mg, 0.180 mmol, 1 eq) in DCM (2 mL) were added Ac2O (36.7 mg, 0.359 mmol, 2 eq) and TEA (72.8 mg, 0.719 mmol, 4 eq). The mixture was stirred at 25° C. for 1 hour and quenched with addition of K₂CO₃ (30.0 mg) at 25° C. The reaction mixture was filtered, and the filtrate was diluted with H₂O (5 mL) and extracted with EtOAc (5 mL*3). The combined organic layers were washed with brine (5 mL*3), dried over Na₂SO₄, filtered and concentrated under reduced pressure. The residue was purified by flash silica gel chromatography to afford Ex. 49 (59.1 mg, 0.129 mmol, 71.7% yield) as a white solid.

Ex. 49-Ex. 52 were prepared following General Method J using the corresponding starting materials as shown below:

| Starting Material | Ex. # |
|---|---|
| Ex. 40 | Ex. 51 |
| 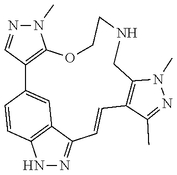 [SMILES: Cc1nn(C)c2c1/C=C/c1n[nH]c3ccc(cc13)-c1cnn(C)c1OCCNC2] | 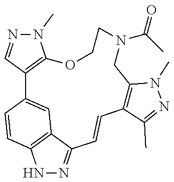 [SMILES: CC(=O)N1CCOc2c(cnn2C)-c2ccc3[nH]nc(c3c2)/C=C/c2c(C)nn(C)c2C1] |
| Ex. 41 | Ex. 52 |
| 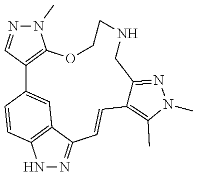 [SMILES: Cc1c2c(nn1C)CNCCOc1c(cnn1C)-c1ccc3[nH]nc(c3c1)/C=C/2] | 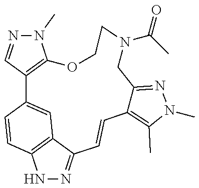 [SMILES: CC(=O)N1CCOc2c(cnn2C)-c2ccc3[nH]nc(c3c2)/C=C/c2c(nn(C)c2C)C1] |
| Ex. 48 | Ex. 53 |
| 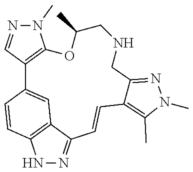 [SMILES: Cc1c2c(nn1C)CNC[C@H](C)Oc1c(cnn1C)-c1ccc3[nH]nc(c3c1)/C=C/2] | 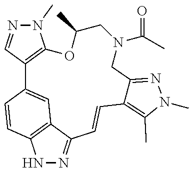 [SMILES: CC(=O)N1Cc2nn(C)c(C)c2/C=C/c2n[nH]c3ccc(cc23)-c2cnn(C)c2O[C@@H](C)C1] |
| Ex. 48 | Ex. 54 |
| 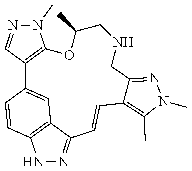 [SMILES: Cc1c2c(nn1C)CNC[C@H](C)Oc1c(cnn1C)-c1ccc3[nH]nc(c3c1)/C=C/2] | 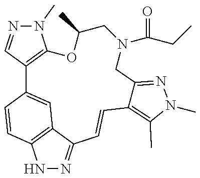 [SMILES: CCC(=O)N1Cc2nn(C)c(C)c2/C=C/c2n[nH]c3ccc(cc23)-c2cnn(C)c2O[C@@H](C)C1] |

### General Method K

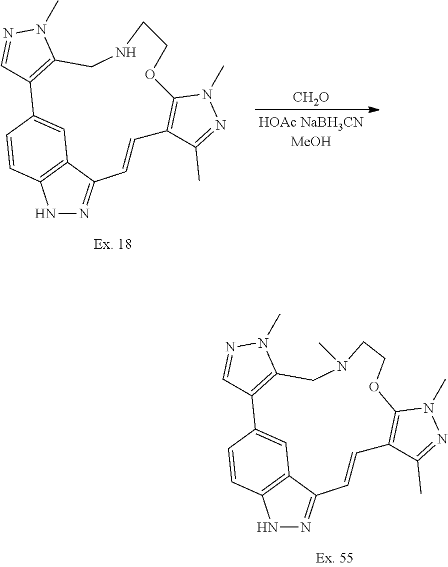 [SMILES: Cc1nn(C)c2c1/C=C/c1n[nH]c3ccc(cc13)-c1cnn(C)c1CNCCO2 ; "CH2O" ; Cc1nn(C)c2c1/C=C/c1n[nH]c3ccc(cc13)-c1cnn(C)c1CN(C)CCO2]

To a mixture of Ex. 18 (90.0 mg, 0.231 mmol, 1 eq) and HCHO (104 mg, 3.47 mmol, 15 eq) in MeOH (1 mL) was added HOAc (13.8 mg, 0.231 mmol, 13.2 uL, 1 eq) at 0° C. The reaction mixture was stirred at 0° C. for 0.5 hour. Then the reaction was added NaBH₃CN (21.7 mg, 0.346 mmol, 1.5 eq). The reaction mixture was stirred at 25° C. for 12 hours. On completion, the reaction mixture was quenched with water (0.05 mL) and concentrated in vacuo. The residue was purified by prep-HPLC afford to Ex. 55 as TFA salt (29.4 mg, 0.0562 mmol, 24% yield) as a white solid.

Ex. 55-Ex. 61 were prepared following General Method J using the corresponding starting materials as shown below:

| Starting Material | Ex. # |
|---|---|
| Ex. 18 | Ex. 55 |
| 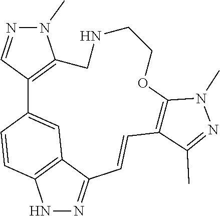 [SMILES: Cc1nn(C)c2c1/C=C/c1n[nH]c3ccc(cc13)-c1cnn(C)c1CNCCO2] | 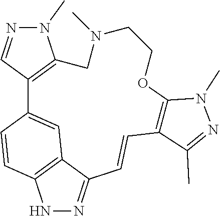 [SMILES: Cc1nn(C)c2c1/C=C/c1n[nH]c3ccc(cc13)-c1cnn(C)c1CN(C)CCO2] |
| Ex. 27 | Ex. 56 |
| 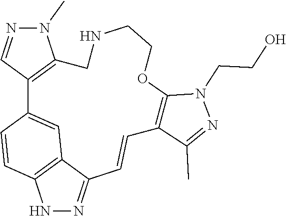 [SMILES: Cc1nn(CCO)c2c1/C=C/c1n[nH]c3ccc(cc13)-c1cnn(C)c1CNCCO2] | 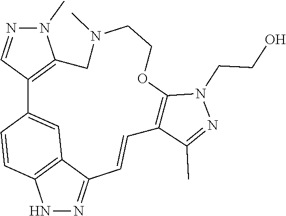 [SMILES: Cc1nn(CCO)c2c1/C=C/c1n[nH]c3ccc(cc13)-c1cnn(C)c1CN(C)CCO2] |
| Ex. 27 | Ex. 57 |
| 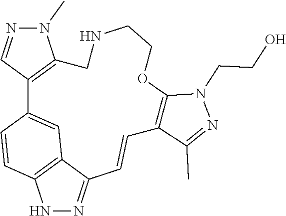 [SMILES: Cc1nn(CCO)c2c1/C=C/c1n[nH]c3ccc(cc13)-c1cnn(C)c1CNCCO2] | 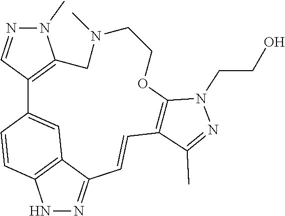 [SMILES: Cc1nn(CCO)c2c1/C=C/c1n[nH]c3ccc(cc13)-c1cnn(C)c1CN(C)CCO2] |
| Ex. 46 | Ex. 58 |
| 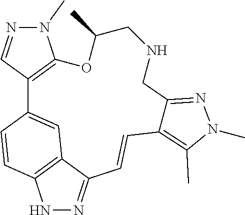 [SMILES: Cc1c2c(nn1C)CNC[C@H](C)Oc1c(cnn1C)-c1ccc3[nH]nc(c3c1)/C=C/2] | 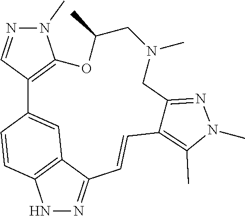 [SMILES: Cc1c2c(nn1C)CN(C)C[C@H](C)Oc1c(cnn1C)-c1ccc3[nH]nc(c3c1)/C=C/2] |
| Ex. 46 | Ex. 59 |
| 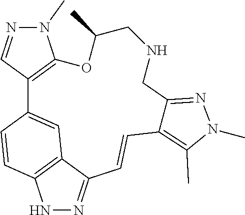 [SMILES: Cc1c2c(nn1C)CNC[C@H](C)Oc1c(cnn1C)-c1ccc3[nH]nc(c3c1)/C=C/2] | 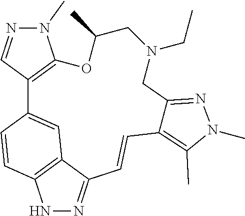 [SMILES: CCN1Cc2nn(C)c(C)c2/C=C/c2n[nH]c3ccc(cc23)-c2cnn(C)c2O[C@@H](C)C1] |
| Ex. 47 | Ex. 60 |
| 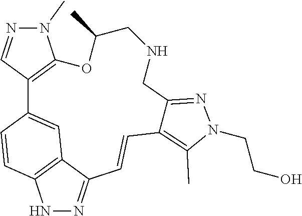 [SMILES: Cc1c2c(nn1CCO)CNC[C@H](C)Oc1c(cnn1C)-c1ccc3[nH]nc(c3c1)/C=C/2] | 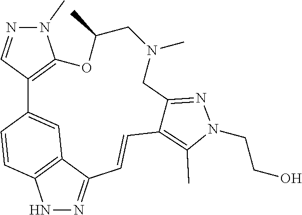 [SMILES: Cc1c2c(nn1CCO)CN(C)C[C@H](C)Oc1c(cnn1C)-c1ccc3[nH]nc(c3c1)/C=C/2] |
| Ex. 48 | Ex. 61 |
| 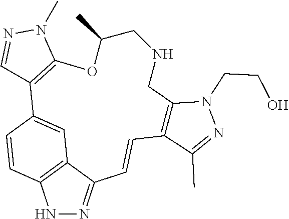 [SMILES: Cc1nn(CCO)c2c1/C=C/c1n[nH]c3ccc(cc13)-c1cnn(C)c1O[C@@H](C)CNC2] | 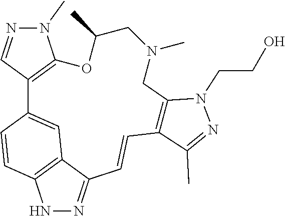 [SMILES: Cc1nn(CCO)c2c1/C=C/c1n[nH]c3ccc(cc13)-c1cnn(C)c1O[C@@H](C)CN(C)C2] |

### Preparation of (19E)-8,16,18-trimethyl-2,11,12,16-tetrahydro-8H, 10H-3,5-ethenotripyrazolo[3,4-h:3′,4′-l:4″,3″-p][1,7,4]dioxazacycloheptadecin-13(14H)-one (Ex. 62)

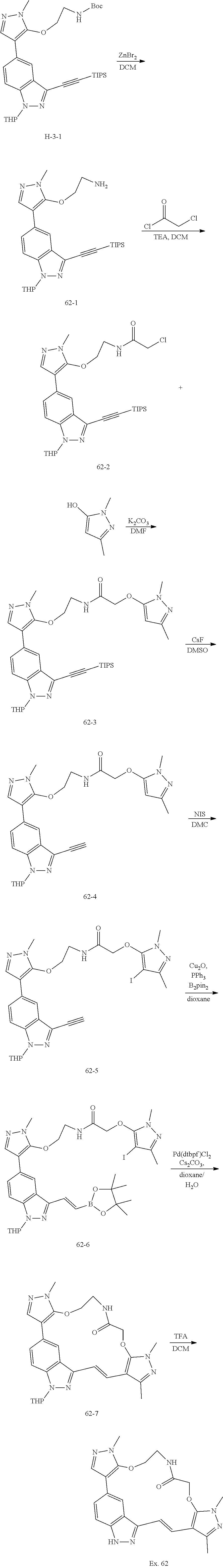

Step 1. To a mixture of tert-butyl N-[2-[2-methyl-4-[1-tetrahydropyran-2-yl-3-(2-triisopropylsilylethynyl) indazol-5-yl]pyrazol-3-yl]oxyethyl]carbamate (4.60 g, 7.40 mmol, 1 eq) in DCM (60 mL) was added ZnBr₂ (8.33 g, 36.9 mmol, 1.85 mL, 5 eq). The reaction mixture was stirred at 25° C. for 16 hour. On completion, the residue was diluted with water (60 mL) and extracted with EA (2×70 mL). The combined organic layers was dried over Na₂SO₄, filtered and concentrated in vacuo to afford 2-[2-methyl-4-[1-tetrahydropyran-2-yl-3-(2-triisopropylsilylethynyl) indazol-5-yl]pyrazol-3-yl]oxyethanamine (4.00 g, 80% yield) as brown solid. LCMS: m/z 522.4 (M+1)

Step 2. To a mixture of 2-[2-methyl-4-[1-tetrahydropyran-2-yl-3-(2-triisopropylsilylethynyl) indazol-5-yl]pyrazol-3-yl]oxyethanamine (2.40 g, 4.60 mmol, 1 eq) and 2-chloroacetyl chloride (779 mg, 6.90 mmol, 548 μL, 1.5 eq) in DCM (20 mL) was added TEA (465 mg, 4.60 mmol, 1 eq) at 0° C. The reaction mixture was stirred at 0° C. for 1 hour. On completion, the residue was diluted with water (50 mL) and extracted with EA (2×50 mL). The combined organic layers were dried over Na₂SO₄, filtered and concentrated in vacuo. The residue was purified by column chromatography to afford 2-chloro-N-[2-[2-methyl-4-[1-tetrahydropyran-2-yl-3-(2-triisopropylsilylethynyl) indazol-5-yl]pyrazol-3-yl]oxyethyl]acetamide (1.50 g, 2.51 mmol, 54% yield) as yellow oil. ¹H NMR (400 MHz, DMSO-d₆) δ 8.47 (t, J=5.6 Hz, 1H), 7.86-7.82 (m, 1H), 7.74-7.68 (m, 3H), 5.90 (dd, J=2.0, 9.6 Hz, 1H), 4.10-4.06 (m, 2H), 4.03 (q, J=7.2 Hz, 1H), 3.96 (t, J=5.6 Hz, 2H), 3.73-3.69 (m, 3H), 3.47-3.41 (m, 2H), 2.39-2.32 (m, 1H), 2.07-1.95 (m, 3H), 1.82-1.70 (m, 1H), 1.64-1.56 (m, 2H), 1.19-1.15 (m, 21H).

Step 3. To a mixture of 2-chloro-N-[2-[2-methyl-4-[1-tetrahydropyran-2-yl-3-(2-triisopropylsilylethynyl) indazol-5-yl]pyrazol-3-yl]oxyethyl]acetamide (1.40 g, 2.34 mmol, 1 eq) and 2,5-dimethylpyrazol-3-ol (341 mg, 3.04 mmol, 1.3 eq) in DMF (2 mL) was added K₂CO₃ (970 mg, 7.02 mmol, 3 eq). The reaction mixture was stirred at 40° C. for 12 hours. On completion, the residue was diluted with water (30 mL) and extracted with EA (2×30 mL). The combined organic layers were dried over Na₂SO₄, filtered and concentrated in vacuo to afford 2-(2,5-dimethylpyrazol-3-yl)oxy-N-|2-|2-methyl-4-|1-tetrahydropyran-2-yl-3-(2-triisopropylsilylethynyl) indazol-5-yl]pyrazol-3-yl]oxyethyl]acetamide (1.40 g, 2.08 mmol, 88% yield) as brown oil. LCMS: m/z 674.9 (M+1).

Step 4. To a mixture of 2-(2,5-dimethylpyrazol-3-yl)oxy-N-[2-[2-methyl-4-[1-tetrahydropyran-2-yl-3-(2-triisopropylsilylethynyl) indazol-5-yl]pyrazol-3-yl]oxyethyl]acetamide (1.30 g, 1.93 mmol, 1 eq) in DMSO (10 mL) was added CsF (1.47 g, 9.65 mmol, 5 eq). The reaction mixture was stirred at 25° C. for 1 hour. On completion, the residue was diluted with water (30 mL) and extracted with EA (2×20 mL). The combined organic layers were dried over Na₂SO₄, filtered and concentrated in vacuo. The residue was purified by column chromatography to afford 2-(2,5-dimethylpyrazol-3-yl)oxy-N-[2-[4-(3-ethynyl-1-tetrahydropyran-2-yl-indazol-5-yl)-2-methyl-pyrazol-3-yl]oxyethyl]acetamide (62-4, 560 mg, 1.08 mmol, 56% yield) as yellow solid. ¹H NMR (400 MHz, DMSO-d₆) δ 8.37 (t, J=5.6 Hz, 1H), 7.79 (s, 2H), 7.77 (s, 1H), 7.73-7.69 (m, 1H), 5.84 (dd, J=2.4, 9.6 Hz, 1H), 5.40 (s, 1H), 4.59 (s, 1H), 4.50 (s, 2H), 4.00-3.94 (m, 2H), 3.90-3.83 (m, 1H), 3.77-3.71 (m, 1H), 3.68 (s, 3H), 3.51 (s, 3H), 3.49 (s, 1H), 2.55-2.51 (m, 1H), 2.38-2.29 (m, 1H), 2.02 (s, 1H), 2.01 (s, 3H), 1.99-1.94 (m, 1H), 1.81-1.69 (m, 1H), 1.63-1.54 (m, 2H)

62-4 was converted to Ex. 62 following Step 5-Step 8 in General Method C.

### Preparation of (11E)-1-[(methanesulfonyl)methyl]-19,20-dihydro-1H,8H,18H-10,7,4-(ethan[1]yl[1,2]diylidene)pyrazolo[3,4-f][1,5,12,13]benzodioxadiazacyclooctadecine

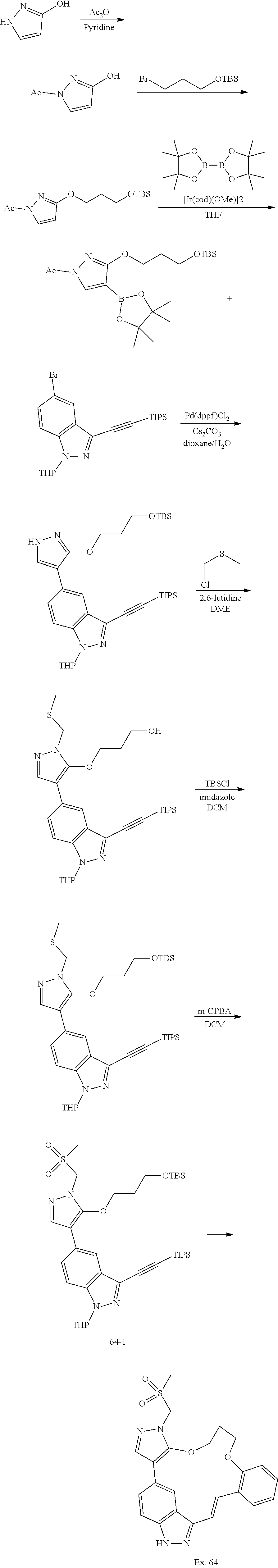

Step 1. A mixture of 1H-pyrazol-3-ol (10.0 g, 119 mmol, 1 eq) in Py (50 mL) was stirred at 95° C. for 0.5 h. To the mixture was added a solution of acetic anhydride (12.1 g, 119 mmol, 11.1 mL, 1 eq) in pyridine (50 mL). The reaction was stirred at 95° C. for 2.5 h. On completion, the reaction mixture was concentrated in vacuo. The residue was diluted with water (80 mL) and extracted with EA (3×120 mL). The combined organic layers were dried over Na₂SO₄, filtered and concentrated in vacuo. The reaction mixture was triturated with PE (30 ml), filtered and dried under vacuo to afford 1-(3-hydroxypyrazol-1-yl)ethanone (12.0 g, 95.15 mmol, 80.00% yield) as a yellow solid. ¹H NMR (400 MHz, DMSO-d₆) δ=10.97 (s, 1H), 8.13 (s, 1H), 6.00 (s, 1H), 2.47 (s, 3H).

Step 2. To a mixture of 1-(3-hydroxypyrazol-1-yl)ethanone (12.0 g, 95.2 mmol, 1 eq) and 3-bromopropoxy-tert-butyl-dimethyl-silane (26.5 g, 105 mmol, 1.1 eq) in DMF (100 mL) was added K₂CO₃ (39.4 g, 285 mmol, 3 eq). The reaction mixture was stirred at 60° C. for 4 h. On completion, the residue was diluted with water (300 mL) and then extracted with EA (3×300 mL). The combined organic layers were dried over Na₂SO₄, filtered and concentrated in vacuo. The residue was purified by column chromatography to afford 1-[3-[3-[tertbutyl(dimethyl)silyl]oxypropoxy]pyrazol-1-yl]ethanone (24.0 g, 80.4 mmol, 84.5% yield) as yellow oil. LCMS: 299.4 (M+1).

Step 3. A mixture of 1-[3-[3-[tert-buty(dimethyl)silyl]oxypropoxy]pyrazol-1-yl]ethanone (5.00 g, 16.7 mmol, 1 eq) and 4,4,5,5-tetramethyl-2-(4,4,5,5-tetramethyl-1,3,2-dioxaborolan-2-yl)-1,3,2-dioxaborolane (12.8 g, 50.3 mmol, 3 eq), 4-tert-butyl-2-(4-tert-butyl-2-pyridyl)pyridine (899 mg, 3.35 mmol, 0.2 eq), bis(1,5-cyclooctadiene)di-p-methoxydiiridium(I) (1.11 g, 1.68 mmol, 0.1 eq) in THF (50 mL) was stirred at 90° C. for 12 h. On completion, the residue was diluted with water (50 mL) and extracted with EA (3×80 mL). The combined organic layer was dried over Na₂SO₄, filtered and concentrated in vacuo. The residue was purified by column chromatography to provide tert-butyl-dimethyl-|3-∥4-(4,4,5, 5-tetramethyl-1,3,2-dioxaborolan-2-yl)-1H-pyrazol-3-yl|oxy|propoxy|silane (6.00 g, 15.7 mmol, 93.7% yield) as yellow solid. LCMS: m/z 383.1 (M+1).

Step 4. A mixture of tert-butyl-dimethyl-[3-[[4-(4,4,5,5-tetramethyl-1,3,2-dioxaborolan-2-yl)-1H-pyrazol-3-yl]oxy]propoxy]silane (7.00 g, 18.3 mmol, 1.0 eq), 2-(5-bromo-1-tetrahydropyran-2-yl-indazol-3-yl)ethynyl-triisopropyl-silane (9.29 g, 20.1 mmol, 1.1 eq), Cs₂CO₃ (17.9 g, 54.9 mmol, 3.0 eq), ditert-butyl(cyclopentyl)phosphane;dichloropalladium;iron (1.19 g, 1.83 mmol, 0.1 eq) in dioxane (150 mL) and H₂O (30 mL) was degassed and purged with N2 for 3 times. The mixture was stirred at 90° C. for 4 hr. under N2 atmosphere. The mixture was dried over anhydrous sodium sulfate, filtered and concentrated. The residue was purified by column chromatography to give tert-butyl-dimethyl-[3-[[4-[1-tetrahydropyran-2-yl-3-(2-triisopropylsilylethynyl) indazol-5-yl]-1H-pyrazol-3-yl]oxy]propoxy]silane (3.00 g, 4.52 mmol, 24.7% yield, 96% purity) as a brown oil. LCMS: m/z 638.0 (M+1)

Step 5. To a solution of tert-butyl-dimethyl-[3-[[4-[1-tetrahydropyran-2-yl-3-(2-triisopropylsilylethynyl) indazol-5-yl]-1H-pyrazol-3-yl]oxy]propoxy]silane (1.50 g, 2.35 mmol, 1.0 eq) in dioxane (20 mL) was added 2,6-dimethylpyridine (505 mg, 4.71 mmol, 2.0 eq) and chloro(methylsulfanyl)methane (1.14 g, 11.8 mmol, 5.0 eq). The mixture was stirred at 110° C. for 4 hr. After cooling down, the mixture was concentrated and the residue was purified by column chromatography to give 3-[2-(methylsulfanylmethyl)-4-[1-tetrahydropyran-2-yl-3-(2-triisopropylsilylethynyl) indazol-5-yl]pyrazol-3-yl]oxypropan-1-ol (500 mg, 0.841 mmol, 35.7% yield) as a colorless oil. LCMS: m/z 583.7 (M+1).

Step 6. To a solution of 3-[2-(methylsulfanylmethyl)-4-[1-tetrahydropyran-2-yl-3-(2-triisopropylsilylethynyl) indazol-5-yl]pyrazol-3-yl]oxypropan-1-ol (1.00 g, 1.72 mmol, 1.0 eq) in DCM (10 mL) was added imidazole (350 mg, 5.15 mmol, 3.0 eq) and TBSCl (388 mg, 2.57 mmol, 1.5 eq). The mixture was stirred at 25° C. for 3 hr, quenched with water (20 mL), and extracted with ethyl acetate (25 mL×3). The combined organic phase was dried over anhydrous sodium sulfate, filtered and concentrated. The residue was purified by column chromatography to give tert-butyl-dimethyl-[3-[2-(methylsulfanylmethyl)-4-[1-tetrahydropyran-2-yl-3-(2-triisopropylsilylethynyl) indazol-5-yl]pyrazol-3-yl]oxypropoxy]silane (900 mg, 1.29 mmol, 75.3% yield) as a colorless oil. LCMS: m/z 697.7 (M+1).

Step 7. To a solution of tert-butyl-dimethyl-[3-[2-(methylsulfanylmethyl)-4-[1-tetrahydropyran-2-yl-3-(2-triisopropylsilylethynyl) indazol-5-yl]pyrazol-3-yl]oxypropoxy]silane (1.20 g, 1.72 mmol, 1.0 eq) in DCM (20 mL) was added m-CPBA (629 mg, 3.10 mmol, 85% purity, 1.8 eq) at 0° C. The mixture was quenched with sat. aqueous Na₂SO₃ (20 mL) and extracted with ethyl acetate (25 mL×3). The combined organic phase was dried over anhydrous sodium sulfate, filtered and concentrated. The residue was purified by column chromatography to give tert-butyl-dimethyl-[3-[2-(methylsulfonylmethyl)-4-[1-tetrahydropyran-2-yl-3-(2-triisopropylsilylethynyl) indazol-5-yl]pyrazol-3-yl]oxypropoxy]silane (64-1, 720 mg, 987 mmol, 57.4% yield) as a colorless oil. LCMS: m/z 729.2 (M+1).

64-1 was converted to Ex. 64following Steps 2,3,4,6,7 and 8 in General Method C using 2-iodophenol in Step 3.

### Preparation of (13R,19E)-8,13,16,18-tetramethyl-2,10,11,13,14,16-hexahydro-8H-3,5-etheno[1,4,7]trioxacycloheptadecino[12,13-c:17,16-c′:8,9-c″ ]tripyrazole (Ex. 65) and (13S,19E)-8,13,16,18-tetramethyl-2,10,11,13,14,16-hexahydro-8H-3,5-etheno[1,4,7]trioxacycloheptadecino[12,13-c:17,16-c′:8,9-c″ ]tripyrazole (Ex. 66)

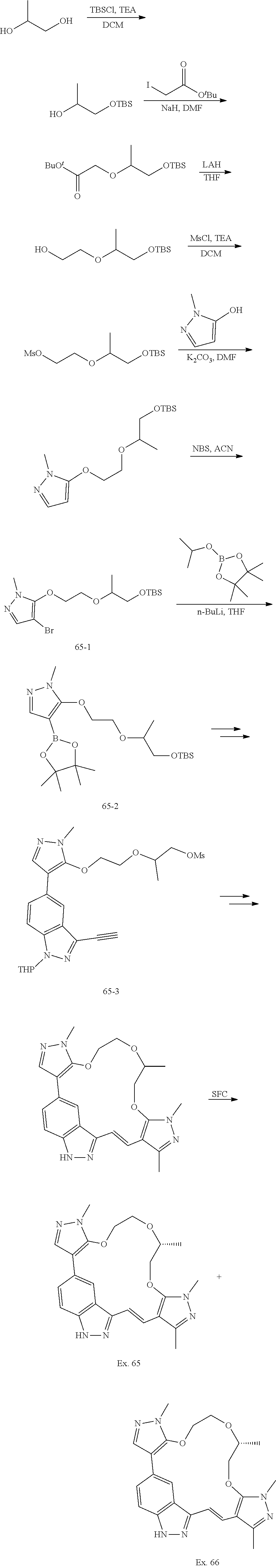

Step 1. To a solution of propane-1,2-diol (25.0 g, 328 mmol, 1 eq) in DCM (250 mL) was added TBSCl (49.5 g, 328 mmol, 1 eq) and imidazole (22.3 g, 328 mmol, 1 eq). The mixture was stirred at 25° C. for 3 hr. On completion, the mixture was quenched with water (400 mL) and extracted with ethyl acetate (400 mL×3), the combined organic phase was dried over anhydrous sodium sulfate, filtered and concentrated to give 1-[tert-butyl(dimethyl)silyl]oxypropan-2-ol (55.0 g, 288 mmol, 87% yield) as a colorless oil. ¹H NMR (400 MHz, DMSO-d₆) δ=4.44 (d, J=4.6 Hz, 1H), 3.58 (td, J=5.7, 11.2 Hz, 1H), 3.47 (dd, J=5.4, 9.8 Hz, 1H), 3.32-3.25 (m, 1H), 1.02 (d, J=6.1 Hz, 3H), 0.86 (s, 9H), 0.03 (s, 6H).

Step 2. To a solution of 1-[tert-butyl(dimethyl)silyl]oxypropan-2-ol (10.0 g, 52.5 mmol, 1 eq) in THF (110 mL) was added NaH (4.20 g, 105 mmol, 60% purity, 2 eq) in batches at 0° C. After addition, the mixture was stirred at this temperature for 30 mins, and then tert-butyl 2-iodoacetate (19.0 g, 78.8 mmol, 1.5 eq) in THE (5 mL) was added dropwise at 0° C. The resulting mixture was stirred at 25° C. for 1.5 hr. On completion, the mixture was quenched with water (100 mL) and extracted with EA (150 mL×3), the combined organic phase was dried over anhydrous sodium sulfate, filtered and the filtrate was concentrated to give a residue. The residue was purified by column chromatography (SiO₂, Petroleum ether/Ethyl acetate=40:1 to 30:1) to give tert-butyl 2-[2-[tert-butyl(dimethyl)silyl]oxy-1-methyl-ethoxylacetate (8.50 g, 27.9 mmol, 53% yield) as a colorless oil. ¹H NMR (400 MHz, CHLOROFORM-d) δ=4.11 (d, J=5.8 Hz, 1H), 4.04-3.98 (m, 1H), 3.65-3.48 (m, 1H), 3.45-3.38 (m, 1H), 1.50-1.45 (m, 10H), 1.18 (dd, J=6.0, 10.4 Hz, 3H), 0.90 (d, J=4.4 Hz, 9H), 0.10-0.05 (m, 6H).

Step 3. To a solution of tert-butyl 2-((1-((tert-butyldimethylsilyl)oxy)propan-2-yl)oxy)acetate (8.50 g, 27.9 mmol, 1 eq) in THF (200 mL) was added LAH (1.06 g, 27.9 mmol, 1 eq) at 0° C. The mixture was stirred at 0° C. for 2 hr. On completion, the mixture was quenched with water (200 mL) and extracted with EA (250 mL×3). The combined organic phase was dried over anhydrous sodium sulfate, filtered and concentrated to give a residue. The residue was purified by column chromatography (SiO₂, Petroleum ether/Ethyl acetate=5:1 to 4:1) to give 2-|2-|tert-butyl(dimethyl)silyl|oxy-1-methyl-ethoxy|ethanol (4.40 g, 18.7 mmol, 67% yield) as a colorless oil

Step 4. To a solution of 2-[2-[tert-butyl(dimethyl)silyl]oxy-1-methyl-ethoxy]ethanol (4.30 g, 18.3 mmol, 1 eq) in DCM (100 mL) was added TEA (9.28 g, 91.7 mmol, 5 eq), and then MsCl (12.6 g, 110 mmol, 6 eq) was added dropwise at 0° C. The mixture was stirred at 25° C. for 2 hr. On completion, the mixture was quenched with water (100 ml) and extracted with DCM (150 mL×3), the combined organic phase was dried over anhydrous sodium sulfate, filtered and concentrated to give 2-[2-[tert-butyl(dimethyl)silyl]oxy-1-methyl-ethoxy]ethyl methanesulfonate (5.70 g, 18.2 mmol, 99% yield) as a yellow oil.

Step 5. To a solution of 2-[2-[tert-butyl(dimethyl)silyl]oxy-1-methyl-ethoxy]ethyl methanesulfonate (5.70 g, 18.2 mmol, 1 eq) and 2-methylpyrazol-3-ol (3.58 g, 36.4 mmol, 2 eq) in DMF (80 mL) was added K₂CO₃ (7.56 g, 54.7 mmol, 3 eq). The mixture was stirred at 60° C. for 12 hr. On completion, the mixture was quenched with water (20 mL) and extracted with ethyl acetate (25 mL×3), the combined organic phase was dried over anhydrous sodium sulfate, filtered and the filtrated was concentrated to give a residue. The residue was purified by column chromatography (SiO₂, Petroleum ether/THF=5:1 to 4:1) to give tert-butyl-dimethyl-[2-[2-(2-methylpyrazol-3-yl)oxyethoxy]propoxy]silane (3.60 g, 11.4 mmol, 62% yield) as a yellow oil. LCMS:315.6 (M+1) m/z

Step 6. To a solution of tert-butyl-dimethyl-[2-[2-(2-methylpyrazol-3-yl)oxyethoxy]propoxy]silane (3.50 g, 11.13 mmol, 1 eq) in MeCN (60 mL) was added NBS (1.98 g, 11.1 mmol, 1 eq). The mixture was stirred at 25° C. for 2 hr. On completion, the mixture was quenched with water (20 mL) and extracted with ethyl acetate (25 mL×3), the combined organic phase was dried over anhydrous sodium sulfate, filtered and the filtrated was concentrated to give a residue. The residue was purified by column chromatography (SiO₂, Petroleum ether/Ethyl acetate=4:1 to 1:1 to give 2 2-[2-(4-bromo-2-methyl-pyrazol-3-yl)oxyethoxy]propoxy-tert-butyl-dimethyl-silane (3.00 g, 7.63 mmol, 68% yield) as a yellow oil. LCMS: m/z 394.4 (M+1)

Step 7. To a solution of 2-[2-(4-bromo-2-methyl-pyrazol-3-yl)oxyethoxy]propoxy-tert-butyl-dimethyl-silane (3.00 g, 7.63 mmol, 1 eq) in THF (80 mL) was added n-BuLi (2.5 M, 9.15 mL, 3 eq) at −78° C. After addition, the mixture was stirred at this temperature for 30 min, and then 2-isopropoxy-4,4,5,5-tetramethyl-1,3,2-dioxaborolane (4.26 g, 22.8 mmol, 3 eq) was added dropwise at −78° C. The mixture was stirred at −78° C. for 1.5 hr. On completion, The mixture was quenched with water (60 ml) and extracted with EA (60 mL×3), the residue was purified by column chromatography (SiO₂, Petroleum ether/THF=8:1 to 6:1) to give tert-butyl-dimethyl-|2-|2-|2-methyl-4-(4,4,5,5-tetramethyl-1,3,2-dioxaborolan-2-yl)pyrazol-3-yl]oxyethoxy]propoxy]silane, 65-1 (2.40 g, 5.45 mmol, 71% yield) as a colorless oil. LCMS: m/z 441.2 (M+1)

The remaining steps were performed according to Method G followed by SFC separation to provide the arbitrarily assigned isomers Ex. 65 and Ex 66.

### Preparation of 2-[(19E)-8,13,18-trimethyl-2,8,10,11,13,14-hexahydro-16H-3,5-etheno[1,4,7]trioxacycloheptadecino[12,13-c:17,16-c′:8,9-c″ ]tripyrazol-16-yl]ethan-1-ol (Ex. 67)

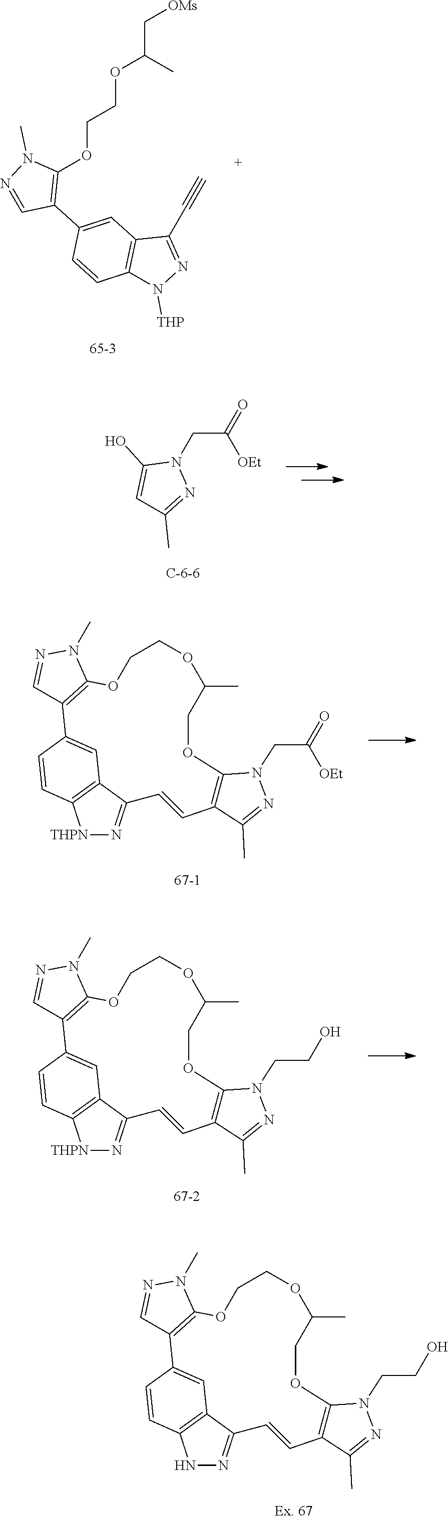

Ex 67 was prepared from 65-3 and C-6-6 via General Method C and General Method D.

### Preparation of (19E)-8,13,18-trimethyl-16-[2-(pyrrolidin-1-yl)ethyl]-2,10,11,13,14,16-hexahydro-8H-3,5-etheno[1,4,7]trioxacycloheptadecino[12,13-c:17,16-c′:8,9-c″ ]tripyrazole (Ex. 68)

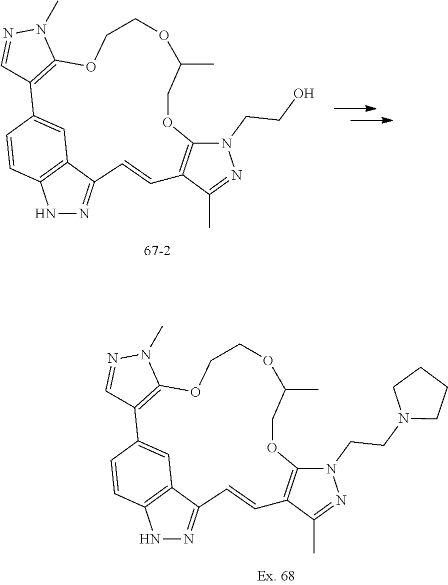 [SMILES: Cc1nn(CCO)c2c1/C=C/c1n[nH]c3ccc(cc13)-c1cnn(C)c1OCCOC(C)CO2 ; Cc1nn(CCN2CCCC2)c2c1/C=C/c1n[nH]c3ccc(cc13)-c1cnn(C)c1OCCOC(C)CO2]

Ex. 68 was prepared from 67-2 in a similar manner to Method F.

Ex. 69 was prepared from D-1-3 and (3S)-pyrrolidin-3-ol via General Method D and General Method F.

### Preparation of 3R)-1-[2-[4-(3-ethynyl-1-tetrahydropyran-2-yl-indazol-5-yl)-2-methyl-pyrazol-3-yl]oxyethyl]-3-(2-iodophenoxy)pyrrolidin-2-one (I-70)

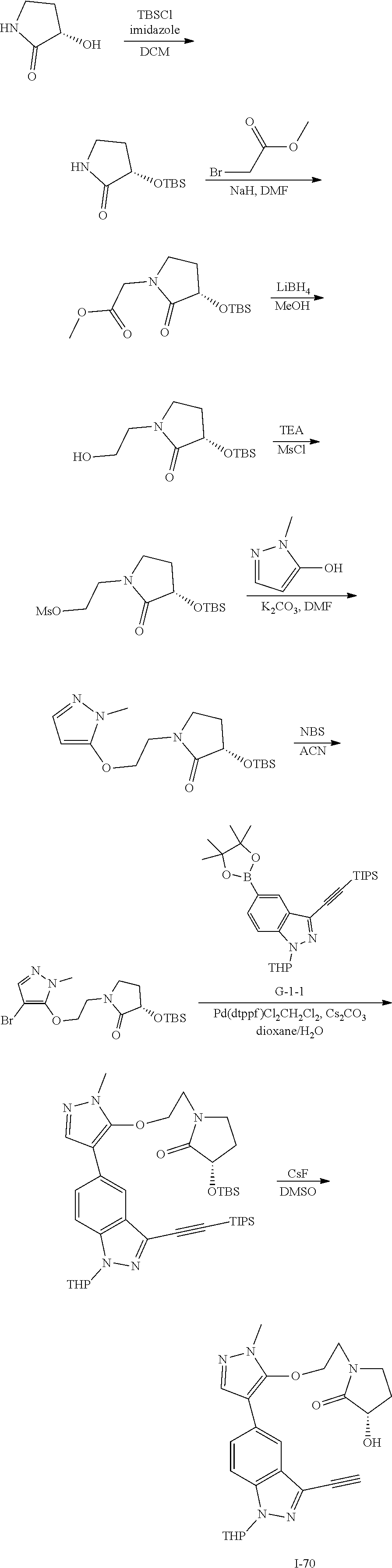

Step 1. A mixture of (3S)-3-hydroxypyrrolidin-2-one (21.3 g, 210 mmol, 1 eq) and TBSCl (47.6 g, 316 mmol, 38.7 mL, 1.5 eq), and imidazole (28.7 g, 421 mmol, 2 eq) in DCM (220 mL) at 0° C., and then the mixture was stirred at 25° C. for 16 h. On completion, the mixture was quenched by H₂O (200 mL) and extracted with EtOAc (80 mL×3). The combined organic layer was dried over anhydrous Na₂SO₄, filtered and the filtrate was concentrate in vacuum to give crude. The residue was purified by combi flash (220 g silica gel column, EA in PE from 0-100%) to give (3S)-3-[tert-butyl(dimethyl)silyl]oxypyrrolidin-2-one (49.0 g, 196 mmol, 93% yield, 86% purity) as a white solid. ¹H NMR (400 MHz, CDCl₃) δ=4.27 (t, J=7.6 Hz, 1H), 3.44-3.36 (m, 1H), 3.27 (td, J=7.6, 9.6 Hz, 1H), 2.38 (dd, J=3.2, 12.8 Hz, 1H), 2.05 (d, J=8.0, 12.8 Hz, 1H), 0.93 (s, 9H), 0.16 (d, J=7.2 Hz, 6H). LCMS: m/z 216.2 (M+1)

Step 2. A mixture of (3S)-3-[tert-butyl(dimethyl)silyl]oxypyrrolidin-2-one (21.0 g, 97.5 mmol, 1 eq) in DMF (189 mL) the mixture was stirred at 0° C. added NaH (5.85 g, 146 mmol, 116 μL, 60% purity, 1.5 eq). Then the mixture was stirred at 0° C. for 0.5 h. Then added methyl 2-bromoacetate (22.4 g, 146 mmol, 13.8 mL, 1.5 eq), and then the mixture was stirred at 0° C. for 0.5 h. On completion, the mixture was quenched by H₂O (400 mL) and extracted with EtOAc (100 mL×3). The combined organic layer was dried over anhydrous Na₂SO₄, filtered and the filtrate was concentrate in vacuum to give crude. The residue was purified by combi flash (220 g silica gel column, THF in PE from 0-100%) to give methyl 2-[(3S)-3-[tert-butyl(dimethyl)silyl]oxy-2-oxo-pyrrolidin-1-yl]acetate (27.8 g, 96.7 mmol, 99% yield) as a colorless oil. ¹H NMR (400 MHz, CDCl₃) δ=4.37 (t, J=8.0 Hz, 1H), 4.29-4.21 (m, 1H), 3.86 (d, J=17.6 Hz, 1H), 3.75-3.70 (m, 3H), 3.44-3.32 (m, 2H), 2.49-2.29 (m, 1H), 2.09-1.92 (m, 1H), 0.91 (s, 9H), 0.15 (d, J=7.2 Hz, 6H). LCMS: m/z 288.4 (M+1)

Step 3. A mixture of methyl 2-[(3S)-3-[tert-butyl(dimethyl)silyl]oxy-2-oxo-pyrrolidin-1-yl]acetate (26.8 g, 93 mmol, 1 eq) in MeOH (480 mL) at 0° C., added LiBH₄ (10.2 g, 466 mmol, 5 eq) and then the mixture was stirred at 0° C. for 2 h. On completion, the mixture was quenched by NH₄Cl (60 mL).The mixture was filtered and the filtrate was concentrate in vacuum to give crude. The residue was purified by combi flash (120 g silica gel column, THF in PE from 0-100%) to give (3S)-3-[tert-butyl(dimethyl)silyl]oxy-1-(2-hydroxyethyl)pyrrolidin-2-one (22.0 g, 84.8 mmol, 91% yield) as a white solid. ¹H NMR (400 MHz, CDCl₃) δ=4.39-4.33 (m, 1H), 3.84-3.75 (m, 2H), 3.51-3.31 (m, 4H), 2.81-2.61 (m, 1H), 2.42-2.25 (m, 1H), 1.99 (d, J=8.4, 12.8 Hz, 1H), 0.92 (s, 9H), 0.16 (d, J=6.8 Hz, 6H). LCMS: m/z 260.2 (M+1)

Step 4. To a solution of (3S)-3-[tert-butyl(dimethyl)silyl]oxy-1-(2-hydroxyethyl)pyrrolidin-2-one (10.5 g, 40.5 mmol, 1 eq) in DCM (105 mL) was added TEA (20.5 g, 202 mmol, 28.2 mL, 5 eq) and MsCl (13.9 g, 121 mmol, 9.40 mL, 3 eq) and then the mixture was stirred at 20° C. for 1 h. On completion, the mixture was quenched by NaHCO₃ (80 mL) and extracted with DCM (70 mL*3). The combined organic layer was dried over anhydrous Na₂SO₄, filtered and the filtrate was concentrate in vacuum to give 2-[(3S)-3-[tert-butyl(dimethyl)silyl]oxy-2-oxo-pyrrolidin-1-yl]ethyl methanesulfonate (13.0 g, 38.5 mmol, 95% yield) as a brown oil.

Step 5. To a solution of 2-[(3S)-3-[tert-butyl(dimethyl)silyl]oxy-2-oxo-pyrrolidin-1-yl]ethyl methanesulfonate (13.0 g, 38.5 mmol, 1 eq) and 2-methylpyrazol-3-ol (4.91 g, 50.1 mmol, 1.3 eq) in DMF (180 mL) was added K₂CO₃ (16.0 g, 116 mmol, 3 eq). The mixture was stirred at 80° C. for 16 h. On completion, the mixture was quenched by H₂O (400 mL) and extracted with EtOAc (100 mL*3). The combined organic layer was dried over anhydrous Na₂SO₄, filtered and the filtrate was concentrate in vacuum to give crude. The residue was purified by combi flash (120 g silica gel column, THF in PE from 0-100%) to give (3S)-3-[tert-butyl(dimethyl)silyl]oxy-1-[2-(2-methylpyrazol-3-yl)oxyethyl]pyrrolidin-2-one (11.5 g, 33.9 mmol, 88% yield) as a colorless oil. ¹H NMR (400 MHz, MeOD-d₄) δ=7.63 (s, 1H), 5.67-5.58 (m, 1H), 4.19-4.11 (m, 2H), 4.06 (d, J=7.2, 11.8 Hz, 1H), 3.91-3.84 (m, 2H), 3.84-3.76 (m, 1H), 3.39 (s, 3H), 2.40 (d, J=6.8, 14.4 Hz, 1H), 2.11-1.98 (m, 1H), 0.89-0.86 (m, 9H), 0.18 (d, J=11.8 Hz, 6H). LCMS: m/z 340.0 (M+1)

Step 6. To a solution of (3S)-3-[tert-butyl(dimethyl)silyl]oxy-1-[2-(2-methylpyrazol-3-yl)oxyethyl]pyrrolidin-2-one (8.20 g, 24.2 mmol, 1 eq) in ACN (82 mL) was added NBS (4.30 g, 24.2 mmol, 1 eq) and 0° C. The mixture was stirred at 0° C. for 2 h. On completion, the reaction mixture was quenched by addition solvent Na₂SO₃ 50 mL at 20° C., and extracted with EtOAc (40 mL*3). The combined organic layer was dried over anhydrous Na₂SO₄, filtered and the filtrate was concentrate in vacuum to give crude. The residue was purified by combi flash (120 g silica gel column, EA in PE from 0-100%) to give (3S)-1-[2-(4-bromo-2-methyl-pyrazol-3-yl)oxyethyl]-3-[tert-butyl(dimethyl)silyl]oxy-pyrrolidin-2-one (4.10 g, 9.80 mmol, 41% yield) as a brown oil. ¹H NMR (400 MHz, CDCl₃) δ=7.30-7.27 (m, 1H), 4.44-4.39 (m, 2H), 4.36-4.31 (m, 1H), 3.79-3.68 (m, 2H), 3.67-3.65 (m, 3H), 3.57-3.50 (m, 1H), 3.46-3.39 (m, 1H), 2.43-2.31 (m, 1H), 2.03-1.92 (m, 1H), 1.89-1.82 (m, 1H), 0.95-0.89 (m, 1H), 0.92 (s, 8H), 0.23-0.11 (m, 6H). LCMS: m/z 419.7 (M+1)

Step 7. A mixture of triisopropyl-[2-[1-tetrahydropyran-2-yl-5-(4,4,5,5-tetramethyl-1,3,2-dioxaborolan-2-yl) indazol-3-yl]ethynyl]silane (3.98 g, 7.82 mmol, 1 eq), (3S)-1-[2-(4-bromo-2-methyl-pyrazol-3-yl)oxyethyl]-3-[tert-butyl(dimethyl)silyl]oxy-pyrrolidin-2-one (3.60 g, 8.60 mmol, 1.1 eq), Cs₂CO₃ (7.65 g, 23.5 mmol, 3 eq) and Pd(dppf)Cl₂-DCM (510 mg, 0.782 mmol, 0.1 eq) in dioxane (80 mL) and H₂O (16 mL) was degassed and purged with N₂ for 3 times, and then the mixture was stirred at 80° C. for 2 h under N2 atmosphere. On completion, the mixture was dried over Na₂SO₄, filtered and concentrated under reduced pressure to give a residue. The residue was purified by combi flash (120 g silica gel column, THE in PE from 0-100%) to give (3S)-3-[tert-butyl(dimethyl)silyl]oxy-1-[2-[2-methyl-4-[1-tetrahydropyran-2-yl-3-(2-triisopropylsilylethynyl) indazol-5-yl]pyrazol-3-yl]oxyethyl]pyrrolidin-2-one (4.15 g, 5.76 mmol, 74% yield) as a black brown oil. LCMS: m/z 720.7 (M+1)

Step 8. A mixture of (3S)-3-[tert-butyl(dimethyl)silyl]oxy-1-[2-[2-methyl-4-[1-tetrahydropyran-2-yl-3-(2-triisopropylsilylethynyl) indazol-5-yl]pyrazol-3-yl]oxyethyl]pyrrolidin-2-one (4.00 g, 5.55 mmol, 1 eq) and CsF (5.06 g, 33.3 mmol, 1.23 mL, 6 eq) in DMSO (40 mL) at 25° C. for 2 h. On completion, the residue was diluted with H₂O 100 mL and extracted with EtOAc (30 mL*3). The combined organic layer was dried over anhydrous Na₂SO₄, filtered and the filtrate was concentrate in vacuum to give crude. The residue was purified by combi flash (12 g silica gel column, MeOH in DCM from 0-100%) to give (3S)-1-[2-[4-(3-ethynyl-1-tetrahydropyran-2-yl-indazol-5-yl)-2-methyl-pyrazol-3-yl]oxyethyl]-3-hydroxy-pyrrolidin-2-one (1.40 g, 3.11 mmol, 56% yield) a brown solid. ¹H NMR (400 MHz, DMSO-d₆) δ=7.87-7.82 (m, 1H), 7.77 (s, 2H), 7.67 (dd, J=1.6, 8.8 Hz, 1H), 6.70-6.58 (m, 1H), 5.58 (dd, J=1.6, 5.6 Hz, 1H), 4.67-4.59 (m, 1H), 4.11 (dd, J=2.0, 8.4 Hz, 1H), 4.04-3.99 (m, 2H), 3.92-3.83 (m, 1H), 3.80-3.72 (m, 1H), 3.68 (s, 3H), 3.60-3.48 (m, 2H), 2.38-2.23 (m, 2H), 2.21-2.15 (m, 2H), 2.07-1.99 (m, 2H), 1.79-1.68 (m, 2H), 1.60 (d, J=3.6 Hz, 2H). LCMS: m/z 450.1(M+1)

### General Method L. Preparation of (10R,16E)-3-methyl-3,5,6,9,10,19-hexahydro-8H-20,22-etheno-7,10-methanodipyrazolo[3,4-i:4,3′-m][1,8,5]benzodioxazacyclooctadecin-25-one (Ex. 70)

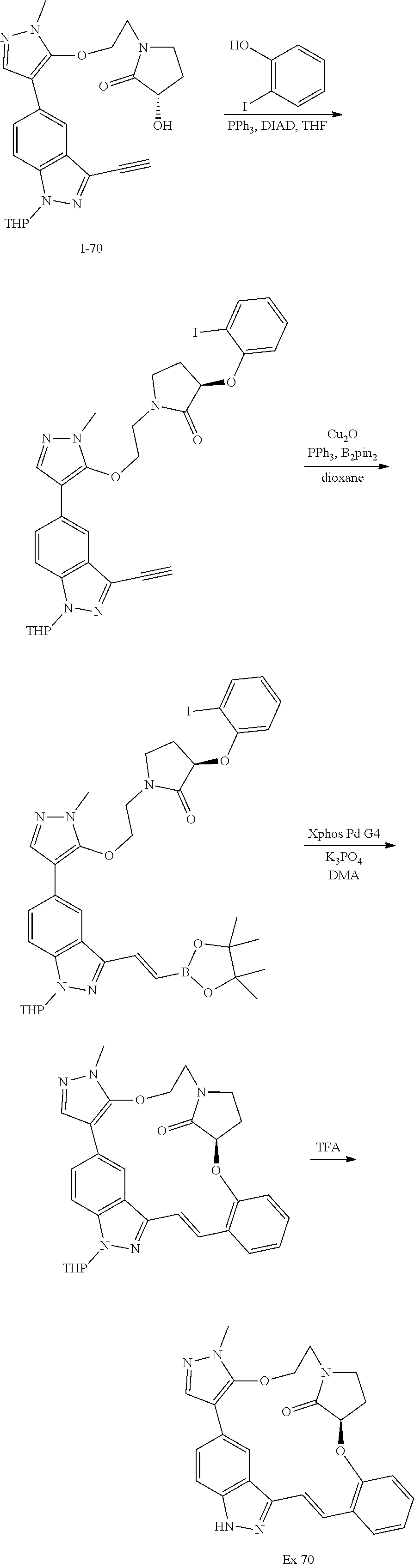

Step 1. The mixture of (3S)-1-[2-[4-(3-ethynyl-1-tetrahydropyran-2-yl-indazol-5-yl)-2-methyl-pyrazol-3-yl]oxyethyl]-3-hydroxy-pyrrolidin-2-one (1.30 g, 2.89 mmol, 1 eq), 2-iodophenol (1.40 g, 6.36 mmol, 718 μL, 2.2 eq) and PPh₃ (1.67 g, 6.36 mmol, 2.2 eq) in 2-MeTHF (65 mL) was stirred at 25° C. for 30 min, then DIAD (1.29 g, 6.36 mmol, 1.24 mL, 2.2 eq) was added dropwise to the mixture at 0° C., the resulting mixture was stirred for another 2 h at 25° C. under N2. On completion, the mixture was filtered and concentrated under reduced pressure to give a residue. The residue was purified by combi flash (40 g silica gel column, THE in PE from 0-100%) to give (3R)-1-[2-[4-(3-ethynyl-1-tetrahydropyran-2-yl-indazol-5-yl)-2-methyl-pyrazol-3-yl]oxyethyl]-3-(2-iodophenoxy)pyrrolidin-2-one (1.35 g, 2.07 mmol, 72% yield) as a light yellow solid. ¹H NMR (400 MHz, CDCl₃) δ=7.84 (s, 1H), 7.78-7.75 (m, 1H), 7.68-7.64 (m, 1H), 7.60-7.54 (m, 2H), 7.34-7.29 (m, 1H), 7.27-7.23 (m, 1H), 6.80-6.74 (m, 1H), 5.73 (d, J=3.2, 9.2 Hz, 1H), 4.86-4.79 (m, 1H), 4.16-4.09 (m, 2H), 4.08-3.95 (m, 2H), 3.78 (s, 3H), 3.68 (t, J=5.2 Hz, 3H), 3.49-3.42 (m, 1H), 2.60-2.46 (m, 2H), 2.34 (dd, J=2.8, 13.6 Hz, 1H), 2.21-2.13 (m, 1H), 2.13-2.06 (m, 1H), 1.81-1.64 (m, 4H). LCMS: m/z 651.9 (M+1)

Step 2. A mixture of (3R)-1-[2-[4-(3-ethynyl-1-tetrahydropyran-2-yl-indazol-5-yl)-2-methyl-pyrazol-3-yl]oxyethyl]-3-(2-iodophenoxy)pyrrolidin-2-one (650 mg, 0.998 mmol, 1 eq), 4,4,5,5-tetramethyl-2-(4,4,5,5-tetramethyl-1,3,2-dioxaborolan-2-yl)-1,3,2-dioxaborolane (507 mg, 2.00 mmol, 2 eq), triphenylphosphine (288 mg, 1.10 mmol, 1.1 eq), Cu₂O (71.4 mg, 0.499 mmol, 51.0 uL, 0.5 eq) in dioxane (26 mL) was degassed and purged with N₂ for 3 times, and then the mixture was stirred at 70° C. for 4 h under N₂ atmosphere. On completion, the mixture was filtered and concentrated under reduced pressure to give a residue. The residue was purified by combi flash (20 g silica gel column, THF in PE from 0-100%) to give (3R)-3-(2-iodophenoxy)-1-|2-|2-methyl-4-|1-tetrahydropyran-2-yl-3-|(E)-2-(4,4,5,5-tetramethyl-1,3,2-dioxaborolan-2-yl)vinyl]indazol-5-yl]pyrazol-3-yl]oxyethyl]pyrrolidin-2-one (475 mg, 0.61 mmol, 61% yield) as a brown oil.

Step 3. A mixture of (3R)-3-(2-iodophenoxy)-1-[2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxyethyl]pyrrolidin-2-one (450 mg, 0.577 mmol, 1 eq), Xphos Pd G4 (49.7 mg, 0.058 mmol, 0.1 eq), K₃PO₄ (367 mg, 1.73 mmol, 3 eq) in DMA (23 mL) was degassed and purged with N₂ for 3 times, and then the mixture was stirred at 80° C. for 2 h under N₂ atmosphere. On completion, the reaction mixture was quenched by addition solvent H₂O 40 mL at 20° C., and extracted with EtOAc (30 mL*3). The combined organic layer was dried over anhydrous Na₂SO₄, filtered and the filtrate was concentrate in vacuum to give crude. The residue was purified by combi flash (4 g silica gel column, MeOH in DCM from 0-100%) to give (13R,21E)-5-methyl-25-tetrahydropyran-2-yl-7,14-dioxa-4,5,10,24,25-pentazahexacyclo[21.5.2.110,13.02,6.015,20.026,30]hentriaconta-1(29),2(6),3,15(20),16,18,21,23,26(30),27-decaen-31-one (110 mg, 0.209 mmol, 36% yield) as a brown oil.

Step 4. To a solution of (13R,21E)-5-methyl-25-tetrahydropyran-2-yl-7,14-dioxa-4,5,10,24,25-pentazahexacyclo[21.5.2.110,13.02,6.015,20.026,30]hentriaconta-1(29),2(6),3,15(20),16,18,21,23,26(30),27-decaen-31-one (100 mg, 0.190 mmol, 1 eq) in DCM (5 mL) was added TFA (7.70 g, 67.5 mmol, 5.00 mL, 355 eq). The mixture was stirred at 20° C. for 1 h. On completion the mixture was filtered and concentrated under reduced pressure to give a residue. The residue was purified by combi flash (4 g silica gel column, MeOH in DCM from 0-10%) to give (13R,21E)-5-methyl-7,14-dioxa-4,5,10,24,25-pentazahexacyclo[21.5.2.110,13.02,6.015,20.026,30]hentriaconta-1(29),2(6),3,15(20),16,18,21,23,26(30),27-decaen-31-one (31.03 mg, 0.065 mmol, 34% yield, 92.9% purity) a yellow solid. ¹H NMR (400 MHz, MeOD-d₄) δ=8.19 (s, 1H), 7.77-7.71 (m, 1H), 7.70-7.65 (m, 2H), 7.64-7.59 (m, 1H), 7.59-7.52 (m, 2H), 7.36-7.28 (m, 1H), 7.18 (d, J=8.0 Hz, 1H), 7.10 (t, J=7.6 Hz, 1H), 5.05 (d, J=6.0 Hz, 1H), 4.45-4.38 (m, 1H), 4.36-4.26 (m, 1H), 4.15-4.04 (m, 2H), 3.85 (s, 3H), 3.57 (t, J=8.8 Hz, 1H), 3.46-3.40 (m, 1H), 2.45-2.32 (m, 1H), 2.27-2.18 (m, 1H)

### Preparation of 2-(5-bromo-7-fluoro-1-tetrahydropyran-2-yl-indazol-3-yl)ethynyl-triisopropyl-silane (I-71)

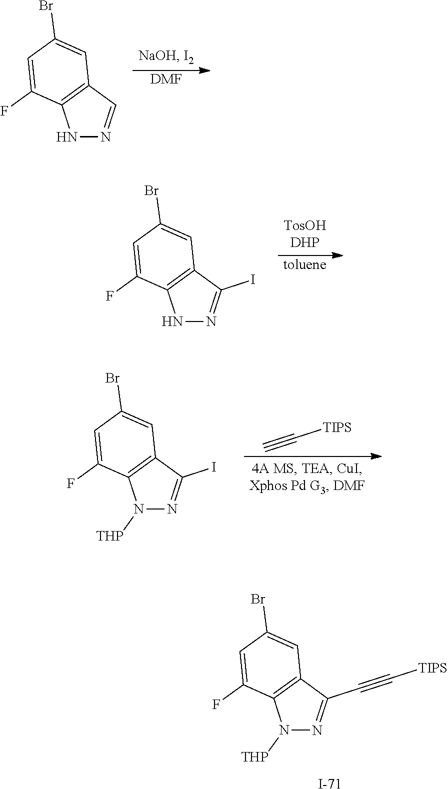

Step 1. To mixture of 5-bromo-7-fluoro-1H-indazole (5.00 g, 23.3 mmol, 1 eq) and I₂ (11.8 g, 46.51 mmol, 2 eq) in DMF (100 mL) was added NaOH (2.79 g, 69.8 mmol, 3 eq), the mixture was stirred at 25° C. for 2 h. On completion, the mixture was quenched with water (300 mL) and extracted with ethyl acetate (125 mL×3), the combined organic phase was dried over anhydrous sodium sulfate, filtered and concentrated to give a residue. The residue was purified by column chromatography (SiO₂, Petroleum ether/Ethyl acetate=10:1) to give 5-bromo-7-fluoro-3-iodo-1H-indazole (7.50 g, 22.0 mmol, 95% yield) as a yellow solid. LCMS: m/z 340.4 (M+1).

Step 2. To a solution of 5-bromo-7-fluoro-3-iodo-1H-indazole (6.50 g, 19.1 mmol, 1 eq) in toluene (65 mL) was added 3,4-dihydro-2H-pyran (3.98 g, 47.3 mmol, 2.48 eq) and DHP (321 mg, 3.81 mmol, 0.2 eq), the mixture was stirred at 90° C. for 16 h. On completion, the mixture was concentrated to give a residue. The residue was purified by column chromatography (SiO₂, PE:THF=10:1) to give 5-bromo-7-fluoro-3-iodo-1-tetrahydropyran-2-yl-indazole (9.00 g, 18.9 mmol, 99% yield, 89% purity) as a yellow solid.

Step 3. To a mixture of 5-bromo-7-fluoro-3-iodo-1-tetrahydropyran-2-yl-indazole (3.00 g, 7.06 mmol, 1 eq), ethynyl(triisopropyl)silane (2.57 g, 14.1 mmol, 2 eq), TEA (10.9 g, 108 mmol, 15 eq), XPhos Pd G3 (597 mg, 0.706 mmol, 0.1 eq) and CuI (67.2 mg, 0.353 mmol, 0.05 eq) in ACN (30 mL) and stirred at 25° C. for 3 h under N₂. On completion, the mixture was quenched with water (80 mL) and extracted with ethyl acetate (35 mL×3), the combined organic phase was dried over anhydrous sodium sulfate, filtered and concentrated to give a residue. The residue was purified by column chromatography (SiO₂, PE:THF=10:1) to give 2-(5-bromo-7-fluoro-1-tetrahydropyran-2-yl-indazol-3-yl)ethynyl-triisopropyl-silane (4.00 g, 7.01 mmol, 99% yield, 84% purity) as a yellow oil. LCMS: m/z 480.8 (M+1).

### Preparation of (19E)-22-fluoro-8,16,18-trimethyl-2,10,11,13,14,16-hexahydro-8H-3,5-etheno[1,4,7]trioxacycloheptadecino[12,13-c:17,16-c′:8,9-c″ ]tripyrazole (Ex. 71)

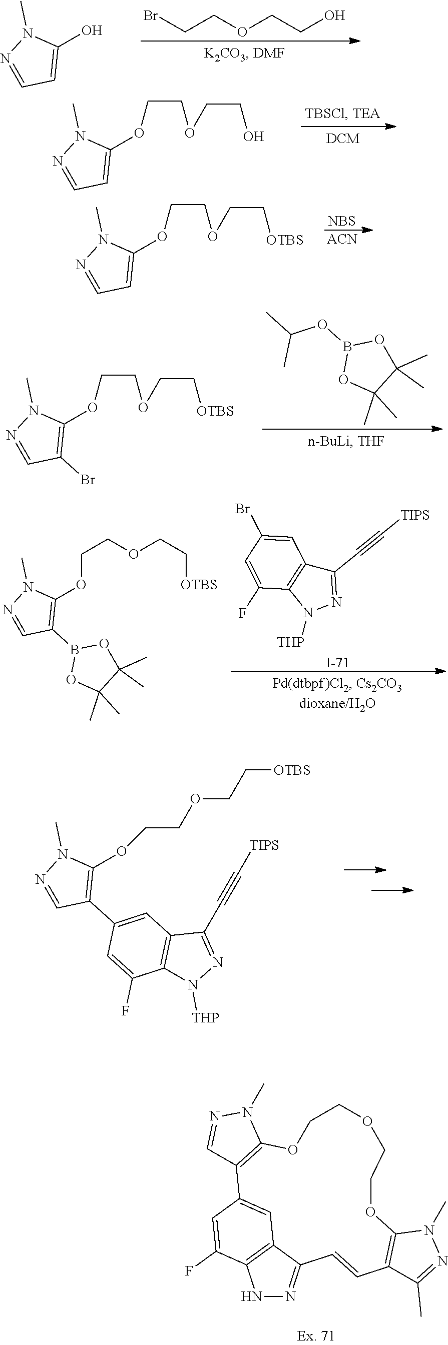

Step 1. To a mixture of 2-methylpyrazol-3-ol (18.2 g, 185 mmol, 1 eq), 2-(2-bromoethoxy)ethanol (47.0 g, 278 mmol, 1.5 eq) and K₂CO₃ (76.9 g, 556 mmol, 3 eq) in DMF (200 mL), the mixture was stirred at 80° C. for 16 h. On completion, the mixture was quenched with water (600 mL) and extracted with DCM: MeOH (10:1) (250 mL×3), the combined organic phase was dried over anhydrous sodium sulfate, filtered and concentrated to give a residue. The residue was purified by column chromatography (SiO₂, MeOH:DCM=10:1) to give 2-[2-(2-methylpyrazol-3-yl)oxyethoxy]ethanol (25.0 g, 134 mmol, 72% yield) as a yellow oil. LCMS: m/z 186.9 (M+1).

Step 2. To a mixture of 2-[2-(2-methylpyrazol-3-yl)oxyethoxy]ethanol (25.0 g, 134 mmol, 1 eq) and imidazole (27.4 g, 403 mmol, 3 eq) in DCM (250 mL) was added TBSCl (28.3 g, 188 mmol, 1.4 eq), the mixture was stirred at 25° C. for 3 h. On completion, the mixture was quenched with water (800 mL) and extracted with DCM (250 mL×3), the combined organic phase was dried over anhydrous sodium sulfate, filtered and concentrated to give tert-butyl-dimethyl-[2-[2-(2-methylpyrazol-3-yl)oxyethoxy]ethoxy]silane (45.0 g, crude) as a yellow oil. LCMS: m/z 301.6 (M+1).

Steps 3-4 were performed in a similar manner to those of steps 2-3 for the synthesis of intermediate H-2-1.

Step 5. To a mixture of tert-butyl-dimethyl-[2-[2-[2-methyl-4-(4,4,5,5-tetramethyl-1,3,2-dioxaborolan-2-yl)pyrazol-3-yl]oxyethoxy]ethoxy]silane (2.00 g, 4.69 mmol, 1 eq), 2-(5-bromo-7-fluoro-1-tetrahydropyran-2-yl-indazol-3-yl)ethynyl-triisopropyl-silane (2.70 g, 5.63 mmol, 1.2 eq), Cs₂CO₃ (4.58 g, 14.1 mmol, 3 eq) and Pd(dppf)Cl₂·CH₂Cl₂ (383 mg, 0.469 mmol, 0.1 eq) in dioxane (40 mL) and H₂O (8 mL), the mixture was stirred at 90° C. for 16 h under N₂. On completion, the mixture was concentrated to give a residue. The residue was purified by column chromatography (SiO₂, PE:THF=3:1) to give tert-butyl-[2-[2-[4-[7-fluoro-1-tetrahydropyran-2-yl-3-(2-triisopropylsilylethynyl) indazol-5-yl]-2-methyl-pyrazol-3-yl]oxyethoxy]ethoxy]-dimethyl-silane (1.96 g, 2.80 mmol, 60% yield) as a yellow oil. LCMS: m/z 699.9 (M+1).

The remaining steps were performed in a manner similar to those described in General Method G to give Ex. 71.

### Preparation of (17E)-15-(2-methoxyethyl)-8,16-dimethyl-2,11,12,15-tetrahydro-8H,10H-3,5-etheno[1,5]dioxacyclopentadecino[10,11-c:15,14-c′:6,7-c″ ]tripyrazole (I-72)

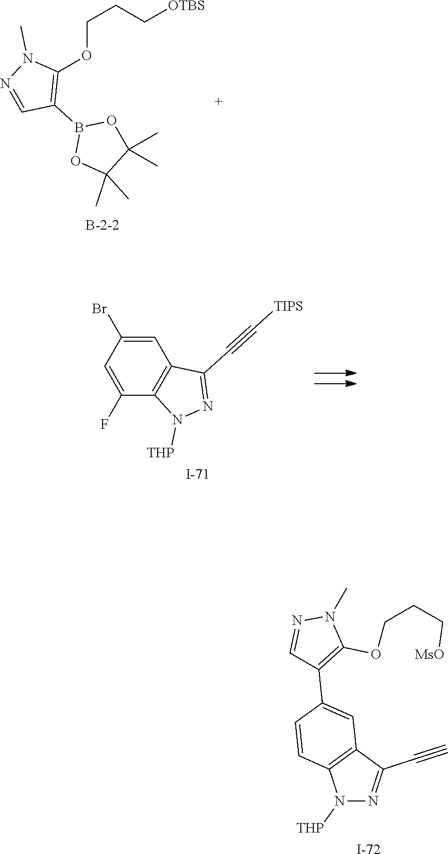

I-72 was synthesized in a manner similar to steps 1-3 in General Method C.

### Preparation of (17E)-15-(2-methoxyethyl)-8,16-dimethyl-2,11,12,15-tetrahydro-8H,10H-3,5-etheno[1,5]dioxacyclopentadecino[10,11-c:15,14-c′:6,7-c″ ]tripyrazole (Ex. 72)

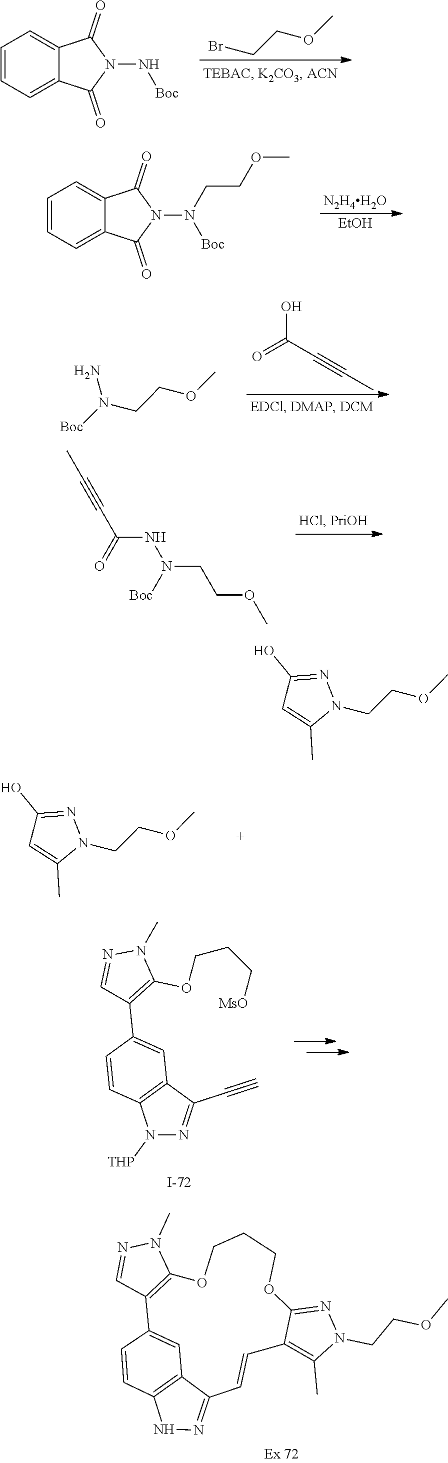

Step 1. To a solution of tert-butyl N-(1,3-dioxoisoindolin-2-yl)carbamate (5 g, 19.0 mmol, 1 eq) in ACN (40 mL) was added 1-bromo-2-methoxy-ethane (5.30 g, 38.1 mmol, 3.58 mL, 2 eq) and benzyl(triethyl)ammonium;chloride (1.74 g, 7.63 mmol, 0.4 eq) and K₂CO₃ (7.90 g, 57.1 mmol, 3 eq). The mixture was stirred at 55° C. for 12 hours. On completion, the mixture was filtered and concentrated to give a residue. The crude product was used into the next step without further purification to give tert-butyl N-(1,3-dioxoisoindolin-2-yl)-N-(2-methoxyethyl)carbamate (6.1 g, 99% yield) as a yellow oil. ¹H NMR (400 MHz, DMSO-d6) δ=8.03-7.89 (m, 4H), 3.71 (t, J=6.0 Hz, 2H), 3.51-3.43 (m, 2H), 3.20-3.12 (m, 3H), 1.45 (s, 4H), 1.24 (s, 5H)

Step 2. To a solution of tert-butyl N-(1,3-dioxoisoindolin-2-yl)-N-(2-methoxyethyl)carbamate (6 g, 18.7 mmol, 1 eq) in EtOH (100 mL) was added N₂H₄·H₂O (6.31 g, 123 mmol, 6.12 mL, 98% purity, 6.59 eq). The mixture was stirred at 80° C. for 1 hour. On completion, the reaction mixture was quenched by addition H₂O 200 mL at 25° C., and then extracted with EA (100 mL*3). The combined organic layers were washed with NaCl (aq.) 100 mL, dried over Na₂SO₄, filtered and concentrated under reduced pressure to give a residue. The crude product was used into the next step without further purification get the title compound tert-butyl N-amino-N-(2-methoxyethyl)carbamate (3.5 g, 98% yield) as a white solid. ¹H NMR (400 MHz, DMSO-d6) δ=4.39 (s, 2H), 3.46-3.42 (m, 2H), 3.41-3.37 (m, 2H), 3.23 (s, 3H), 1.40 (s, 9H)

Step 3. To a solution of but-2-ynoic acid (625 mg, 7.44 mmol, 1 eq) and DMAP (90.8 mg, 0.743 mmol, 0.1 eq) in DCM (5 mL) was added dropwise tert-butyl N-amino-N-(2-methoxyethyl)carbamate (1.5 g, 7.88 mmol, 1.06 eq) at 0° C. And then EDCl (1.57 g, 8.18 mmol, 1.1 eq) was added was in five equal portions over 50 min at 0° C. The resulting mixture was stirred at 25° C. for 1.5 hours. On completion, the reaction mixture was quenched by addition HCl (aq.) 10 mL at 25° C., and then diluted with HCl (aq.) 10 mL and extracted with EA (15 mL*3). The combined organic layers were washed with NaCl (aq.) 10 ml, dried over Na₂SO₄, filtered and concentrated under reduced pressure to give a residue. The crude product was used into the next step without further purification get the title compound tert-butyl N-(but-2-ynoylamino)-N-(2-methoxyethyl)carbamate (1.5 g, 78% yield) as a yellow oil.

Step 4. To a solution of tert-butyl N-(but-2-ynoylamino)-N-(2-methoxyethyl)carbamate (1.5 g, 5.85 mmol, 1 eq) and propan-2-ol (5.89 g, 97.9 mmol, 16.7 eq) was added HCl (4 M, 3.2 mL, 2.2 eq). The mixture was stirred at 63° C. for 30 hours. On completion, the resulting residue was then charged with CH₃CN (100 mL, 10 mL/g) and concentrated in vacuo. The solids were filtered, washed with CH₃CN (2×40 mL), and then the filter liquor dried under vacuum at ambient temperature. The crude was used into the next step without further purification get the title compound 1-(2-methoxyethyl)-5-methyl-pyrazol-3-ol (300 mg, 32% yield) as a yellow oil. ¹H NMR (400 MHz, DMSO-d6) δ=9.39 (s, 1H), 5.22 (s, 1H), 3.90 (t, J=5.6 Hz, 2H), 3.55 (t, J=5.6 Hz, 2H), 3.20 (s, 3H), 2.11 (s, 3H)

The remaining steps were performed in a similar manner to General Method C to give Ex 72.

### Preparation of 3-[(17E)-16-ethyl-8-methyl-2,8,11,12-tetrahydro-10H,14H-3,5-etheno[1,5]dioxacyclopentadecino[10,11-c:15,14-c′:6,7-c″ ]tripyrazol-14-yl]-N,N-dimethylpropan-1-amine (Ex. 73)

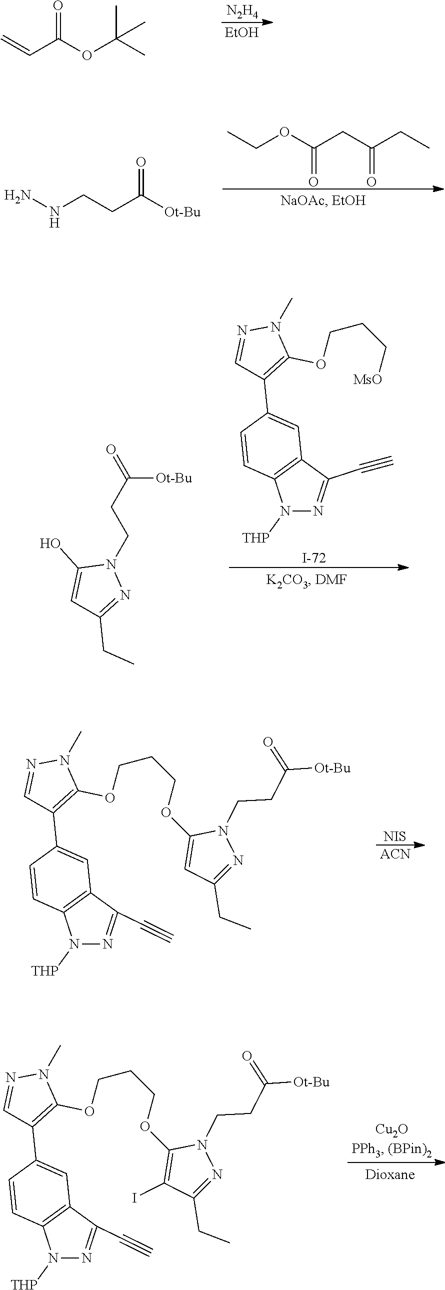

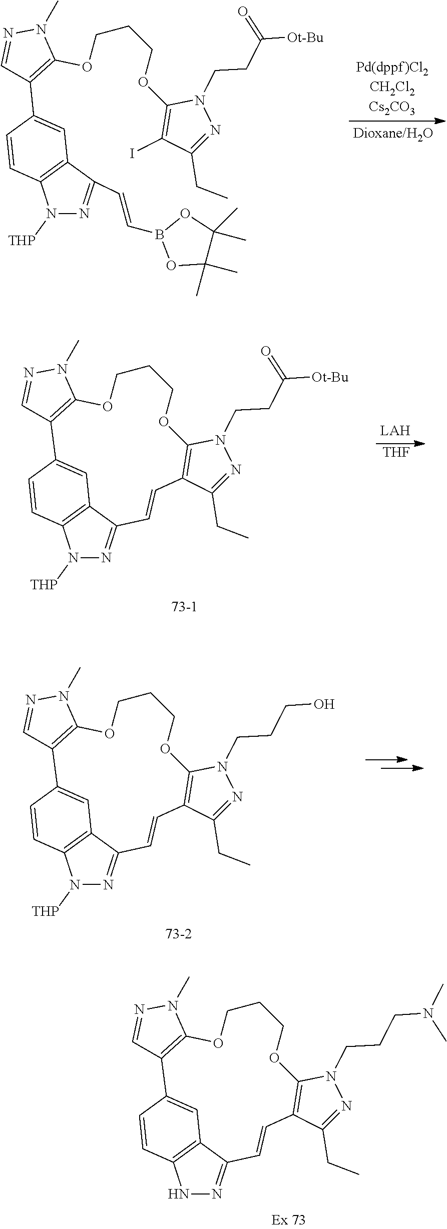

Step 1. To a solution of tert-butyl prop-2-enoate (10.0 g, 78.0 mmol, 11 mL, 1 eq) in EtOH (100 mL) was added N₂H₄·H₂O (4.78 g, 93.6 mmol, 5 mL, 98% purity, 1.2 eq). The mixture was stirred at 60° C. for 2 h. On completion, the reaction mixture was concentrated under reduced pressure to remove solvent. Tert-butyl 3-hydrazinopropanoate (11.7 g, 73.0 mmol, 94% yield) was obtained as yellow oil.

To a solution of tert-butyl 3-hydrazinopropanoate (11.7 g, 73.0 mmol, 1 eq) in EtOH (130 mL) was added NaOAc (8.99 g, 110 mmol, 1.5 eq) and ethyl 3-oxopentanoate (11.6 g, 80.3 mmol, 1.1 eq). The mixture was stirred at 80° C. for 16 h. On completion, the reaction mixture was filtered and concentrated under reduced pressure to give a residue. The residue was purified by column chromatography (SiO₂, DCM/MeOH=1/0 to 0/1). tert-butyl 3-(3-ethyl-5-hydroxy-pyrazol-1-yl)propanoate (17.0 g, 70.6 mmol, 97% yield) was obtained as black brown oil. LCMS: m/z 241.0 (M+1).

Steps 3-6 were done in a similar manner to General Method C.

Step 7. To a solution of tert-butyl 3-[(17E)-15-ethyl-5-methyl-21-tetrahydropyran-2-yl-7,11-dioxa-4,5,13,14,20,21-hexazapentacyclo[17.5.2.02,6.012,16.022,26]hexacosa-1(25),2(6),3,12(16),14,17,19,22(26),23-nonaen-13-yl]propanoate (500 mg, 0.829 mmol, 1 eq) in THF (10 mL) was added LiAlH₄ (94.5 mg, 2.49 mmol, 3 eq) at 0° C. The mixture was stirred at 20° C. for 1 h. On completion, the reaction mixture was quenched by addition MeOH 10 mL at 0° C. The residue was purified by column chromatography (SiO₂, DCM/MeOH=1/0 to 10/1). 3-[(17E)-15-ethyl-5-methyl-21-tetrahydropyran-2-yl-7,11-dioxa-4,5,13,14,20,21-hexazapentacyclo[17.5.2.02,6.012,16.022,26]hexacosa-1(25),2(6),3,12(16),14,17,19,22(26),23-nonaen-13-yl]propan-1-ol (290 mg, 0.544 mmol, 66% yield) was obtained as yellow solid. LCMS: m/z 603.0 (M+1).

The remaining steps were done in a similar manner to General Method F with dimethylamine in step 2 to give Ex. 73.

### Preparation of (rac)-4-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]pyrrolidin-3-ol (Ex. 74)

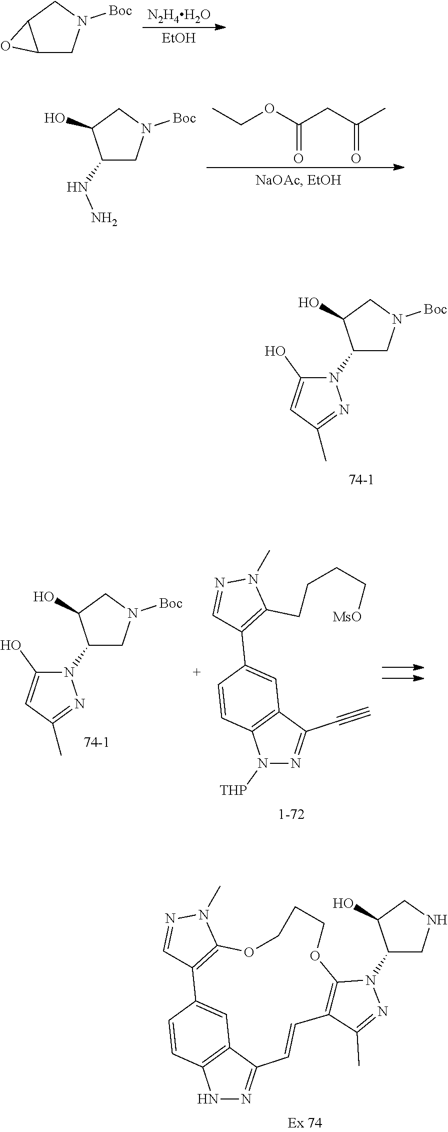

Step 1. To a mixture of tert-butyl 6-oxa-3-azabicyclo[3.1.0]hexane-3-carboxylate (6.70 g, 36.1 mmol, 1 eq) in EtOH (70 mL) was added N₂H₄·H₂O (5.83 g, 114 mmol, 5.66 mL, 98% purity, 3.16 eq) at 0° C. The reaction mixture was stirred at 60° C. for 12 hours. On completion, the reaction mixture was concentrated in vacuo afford to tert-butyl (3S,4S)-3-hydrazino-4-hydroxy-pyrrolidine-1-carboxylate (7.50 g, 34.5 mmol, 95% yield) as yellow oil. ¹H NMR (400 MHz, DMSO-d₆) δ=4.02 (s, 1H), 3.44 (d, J=7.2 Hz, 1H), 3.32-3.26 (m, 2H), 3.18-3.06 (m, 2H), 2.98 (d, J=6.0 Hz, 1H), 1.38 (s, 9H).

Step 2. To a mixture of tert-butyl (3S,4S)-3-hydrazino-4-hydroxy-pyrrolidine-1-carboxylate (7.50 g, 34.5 mmol, 1 eq) and ethyl 3-oxobutanoate (4.94 g, 37.9 mmol, 4.80 mL, 1.1 eq) in EtOH (80 mL) was added NaOAc (4.25 g, 51.7 mmol, 1.5 eq). The reaction mixture was stirred at 80° C. for 12 hours. On completion, the residue was diluted with water (70 mL) and extracted with EA (2×70 mL). The combined organic layers was dried over Na₂SO₄, filtered and concentrated in vacuo to give a residue. The residue was purified by column chromatography afford tert-butyl (3S,4S)-3-hydroxy-4-(5-hydroxy-3-methyl-pyrazol-1-yl)pyrrolidine-1-carboxylate (5.90 g, 20.8 mmol, 60% yield) as yellow solid. ¹H NMR (400 MHz, DMSO-d₆) δ=10.87 (s, 1H), 5.49-5.36 (m, 1H), 5.25-5.11 (m, 1H), 4.45 (d, J=4.4 Hz, 1H), 4.26 (s, 1H), 3.66-3.56 (m, 2H), 3.41 (s, 1H), 3.18-3.12 (m, 1H), 2.02-1.97 (m, 3H), 1.40 (d, J=4.8 Hz, 9H).

The remaining steps were performed in a similar manner to those in General Method C to afford racemic Example 74.

Racemic Ex. 75 was synthesized using Method K starting from racemic Ex. 74

### Preparation of (3R,4S)-4-[(17E)-8,16-dimethyl-2,8,11,12-tetrahydro-10H,14H-3,5-etheno[1,5]dioxacyclopentadecino[10,11-c:15,14-c′:6,7-c′]tripyrazol-14-yl]oxolan-3-ol (Ex. 76) and (3S,4R)-4-[(17E)-8,16-dimethyl-2,8,11,12-tetrahydro-10H,14H-3,5-etheno[1,5]dioxacyclopentadecino[10,11-c:15,14-c′:6,7-c′]tripyrazol-14-yl]oxolan-3-ol (Ex. 77)

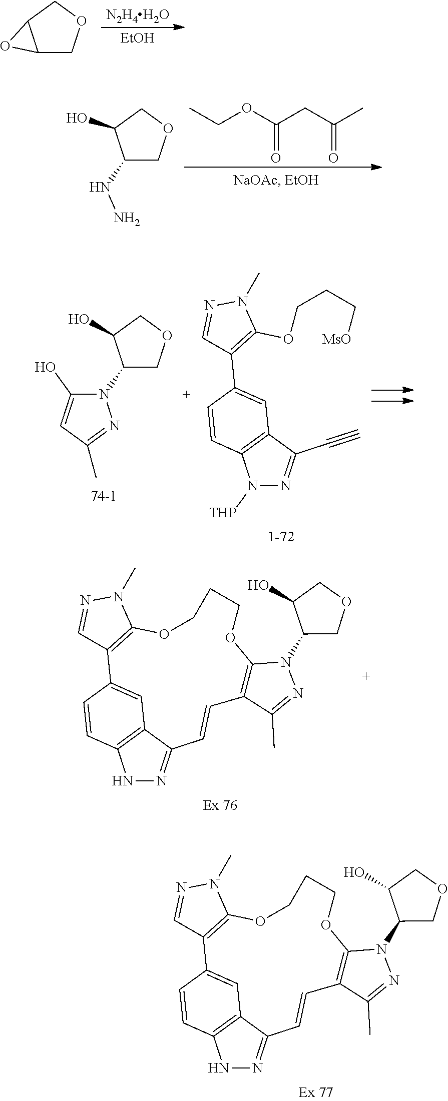

Step 1. To a solution of 3,6-dioxabicyclo[3.1.0]hexane (1.00 g, 11.6 mmol, 1 eq) in EtOH (39 mL) was added N₂H₄·H₂O (1.48 g, 29.0 mmol, 98% purity, 2.5 eq) at 0° C. The reaction was stirred at 25° C. for 10 min, then heated at 60° C. and stirred for 16 h. On completion, the mixture was concentrated to give (3R,4S)-4-hydrazinotetrahydrofuran-3-ol (770 mg, 6.52 mmol, 56% yield) as a white oil. ¹H NMR (400 MHz, DMSO-d₆) δ=4.04-4.02 (m, 1H), 3.79-3.69 (m, 3H), 3.49 (d, J=2.4 Hz, 1H), 3.48-3.45 (m, 2H), 3.44 (d, J=1.6 Hz, 1H), 3.05-3.02 (m, 1H)

Step 2. The mixture of (3R,4S)-4-hydrazinotetrahydrofuran-3-ol (3.05 g, 25.8 mmol, 1 eq), ethyl 3-oxobutanoate (3.36 g, 25.8 mmol, 1 eq) and NaOAc (2.12 g, 25.8 mmol, 1 eq) in EtOH (60 mL) was stirred at 80° C. for 2 h. On completion, the mixture was concentrated to give a residue. The residue was purified by column chromatography (SiO₂, PE:THF=1:3) to give 2-[(3S,4R)-4-hydroxytetrahydrofuran-3-yl]-5-methyl-pyrazol-3-ol, (74-1) (2.65 g, 14.4 mmol, 56% yield) as a yellow oil. ¹H NMR (400 MHz, DMSO-d₆) δ=10.82 (br d, J=1.6 Hz, 1H), 5.35 (br d, J=4.0 Hz, 1H), 5.15 (s, 1H), 4.51-4.45 (m, 1H), 4.37 (br s, 1H), 4.06 (br t, J=8.0 Hz, 1H), 3.98-3.91 (m, 1H), 3.73-3.66 (m, 1H), 3.62-3.54 (m, 1H), 2.00 (s, 3H). LCMS: m/z 184.9 (M+1)

The remaining steps were performed in a similar manner to those in General Method C followed by SFC separation to give the arbitrarily assigned Example 76 and Example 77.

### Preparation of (rac)-2-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]cyclobutan-1-ol (Ex. 78)

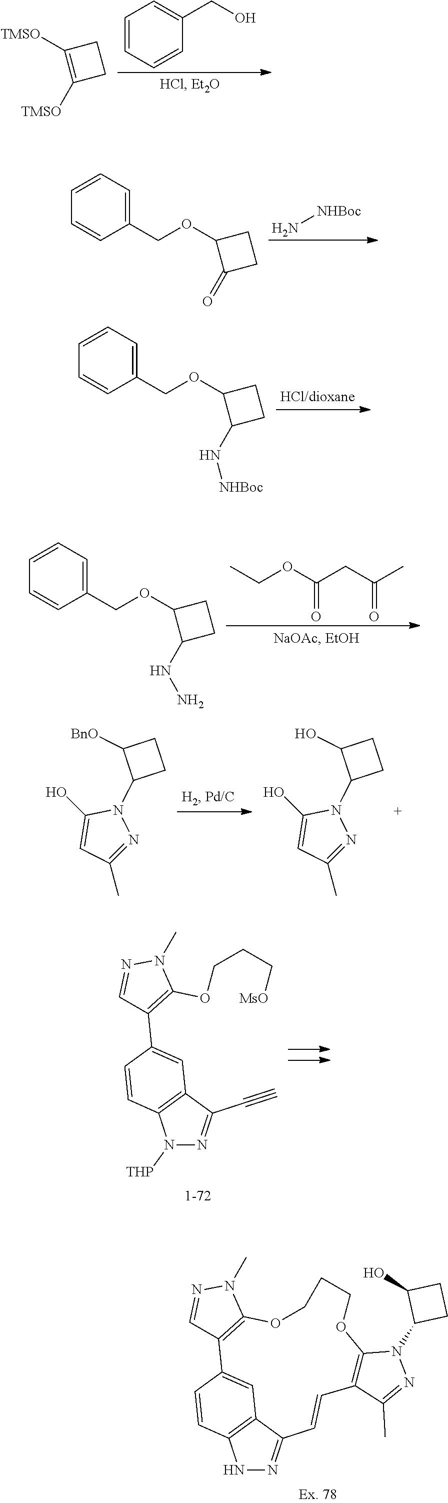

Step 1. A mixture of trimethyl-(2-trimethylsilyloxycyclobuten-1-yl)oxy-silane (5.00 g, 21.7 mmol, 1 eq), phenylmethanol (2.82 g, 26.0 mmol, 1.2 eq), in HCl/dioxane (4 M, 10.8 mL, 2 eq), was stirred at 80° C. for 4 h. On completion, the mixture was filtered and concentrated to give a residue. The residue was purified by column chromatography (SiO₂, Petroleum ether/THF=1:0 to 10:1) to give 2-benzyloxycyclobutanone (3.02 g, 78% yield) as colorless oil.

Step 2. To a mixture of tert-butyl N-aminocarbamate (405 mg, 3.06 mmol, 1.2 eq) and 2-benzyloxycyclobutanone (450 mg, 2.55 mmol, 1 eq) in MeOH (4.5 mL) was added AcOH (153 mg, 2.55 mmol, 146. uL, 1 eq) to adjust pH=6. The mixture was stirred at 25° C. for 0.2 h. Then NaBH₃CN (481 mg, 7.66 mmol, 3 eq) was added to the mixture, the mixture was stirred at 25° C. for 12 h. On completion, the mixture was quenched with water (1 mL) and concentrated to give a residue. The residue was purified by column chromatography (SiO₂, Petroleum ether/THF=1:0 to 4:1) to give tert-butyl N-[(2-benzyloxycyclobutyl)amino]carbamate (372 mg, 1.27 mmol, 50% yield) as a colorless oil. LCMS: m/z 192.9 (M+1-100)

Step 3. To a solution of tert-butyl N-[(2-benzyloxycyclobutyl)amino]carbamate (342 mg, 1.17 mmol, 1 eq) in DCM (3 mL) was added HCl/dioxane (4 M, 7 mL, 22.9 eq). The mixture was stirred at 25° C. for 2 h. On completion, the mixture was concentrated to give (2-benzyloxycyclobutyl)hydrazine (267 mg, crude, HCl) as a red oil. LCMS: m/z 192.9 (M+1)

Step 4. To a solution of (2-benzyloxycyclobutyl)hydrazine (267 mg, 1.17 mmol, 1 eq, HCl) in EtOH (2.7 mL) was added NaOAc (96.0 mg, 1.17 mmol, 1 eq) and ethyl 3-oxobutanoate (152 mg, 1.17 mmol, 1 eq). The mixture was stirred at 80° C. for 2 h. On completion, the mixture was concentrated to give a residue. The residue was purified by column chromatography (SiO₂, Petroleum ether/THF=1:0 to 1:1) to give 2-(2-benzyloxycyclobutyl)-5-methyl-pyrazol-3-ol (92.0 mg, 0.356 mmol, 30% yield) as a red oil. LCMS: m/z 259.1 (M+1)

Step 5. To a solution of 2-(2-benzyloxycyclobutyl)-5-methyl-pyrazol-3-ol (540 mg, 2.09 mmol, 1 eq) in MeOH (20 mL), and HCl/dioxane (2 mL) was added Pd/C (130 mg, 10% purity) under N2. The suspension was degassed under vacuum and purged with H₂ for several times. The mixture was stirred under H₂ (50 psi) at 50° C. for 16 h. On completion, the mixture was concentrated to give 2-(2-hydroxycyclobutyl)-5-methyl-pyrazol-3-ol (475 mg, crude) as a colorless oil. LCMS: m/z 168.9 (M+1)

The remaining steps were performed in a similar manner to those described in General Method C to afford racemic Ex. 78.

### Preparation of 2-[(17E)-8,9,16-trimethyl-8,9,11,12-tetrahydro-3,5-etheno[1,4]dioxacyclopentadecino[11,10-c:15,14-c′:6,7-c″ ]tripyrazol-15(2H)-yl]ethan-1-ol (Ex 79)

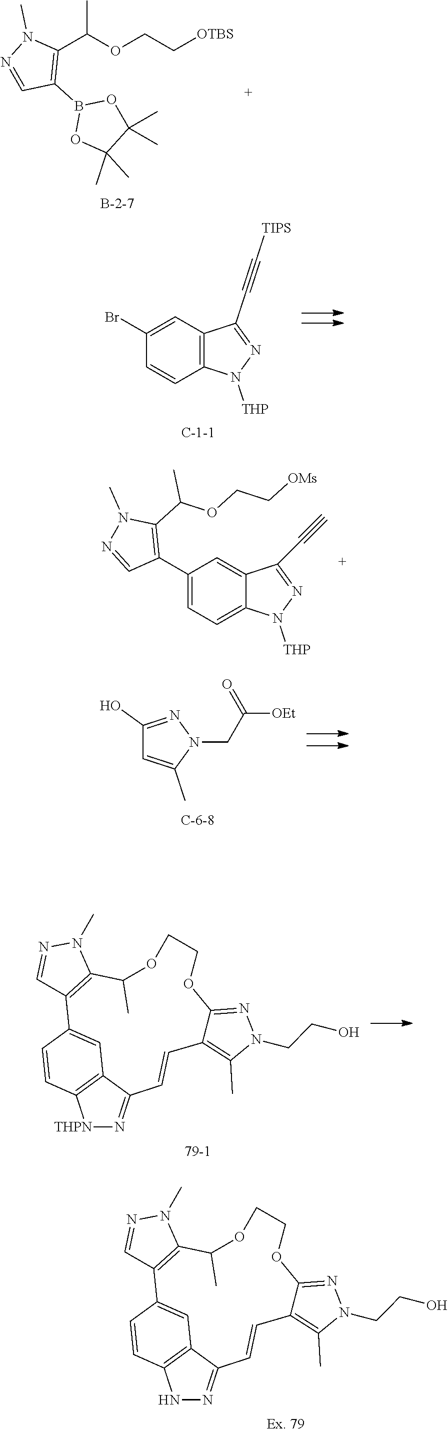

Ex. 79 was synthesized in a manner similar to those described in General Method C and General Method D with the appropriate intermediates shown.

### Synthesis of (17E)-5,15-dimethyl-21-tetrahydropyran-2-yl-7,11-dioxa-4,5,13,14,20,21-hexazapentacyclo [17.5.2.0^{2,6}.0^{12,16}0.0^{22,26}]hexacosa 1(25),2(6),3,12(16),14,17,19,22(26),23-nonaene (I-80)

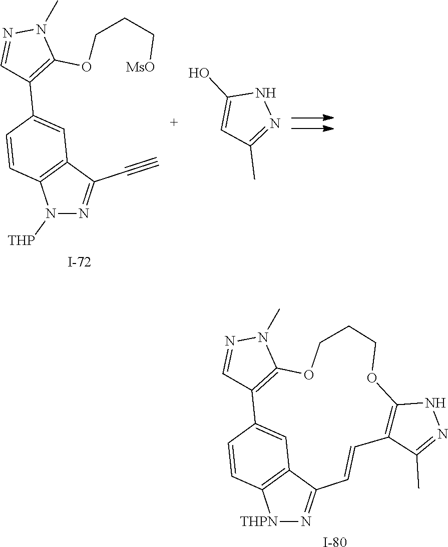

I-80 was prepared in a manner similar to those described in General Method G and Method L. ¹H NMR (400 MHz, CDCl₃) δ=8.60 (s, 1H), 7.80 (s, 1H), 7.64-7.59 (m, 1H), 7.55 (d, J=8.2 Hz, 1H), 6.99 (s, 2H), 5.73-5.64 (m, 1H), 4.58-4.33 (m, 4H), 4.10 (d, J=12.0 Hz, 1H), 3.80 (s, 4H), 2.63-2.51 (m, 4H), 2.38 (s, 2H), 2.17 (d, J=5.2 Hz, 1H), 2.08 (d, J=12.4 Hz, 1H), 1.83-1.75 (m, 2H), 1.67 (s, 1H). LCMS: m/z 461.2 (M+1)

### Preparation of (rac)-4-[(17E)-8,16-dimethyl-2,8,11,12-tetrahydro-10H,14H-3,5-etheno[1,5]dioxacyclopentadecino[10,11-c:15,14-c′:6,7-c′]tripyrazol-14-yl]piperidin-3-ol (Ex. 80) and (rac)-4-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]piperidin-3-ol (Ex. 81)

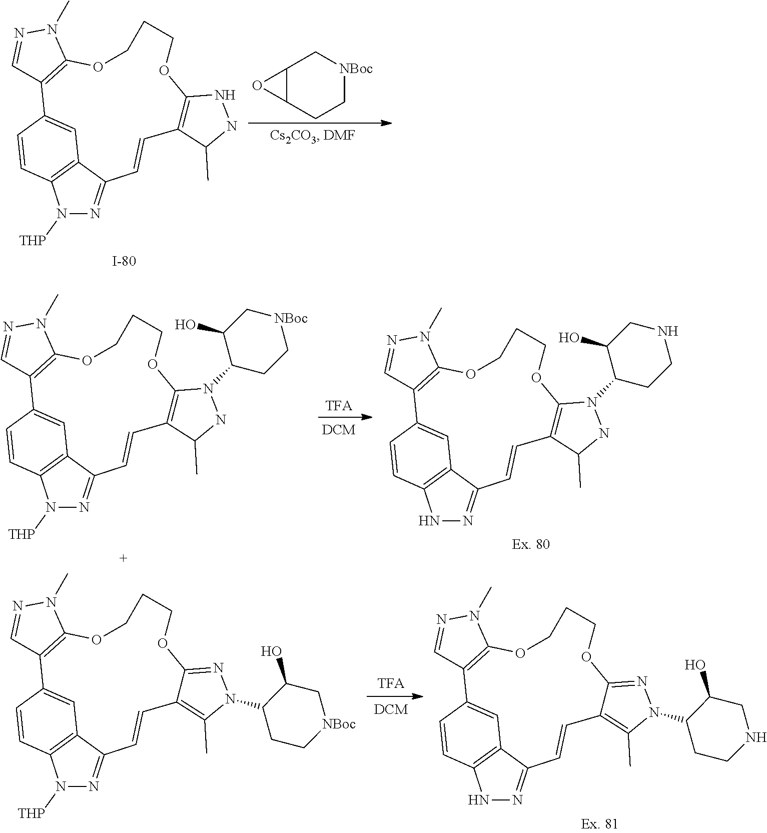

Step 1. To a solution of I-80 (150 mg, 0.325 mmol, 1 eq) in DMF (8 mL) was added Cs₂CO₃ (318 mg, 0.977 mmol, 3 eq) and tert-butyl 7-oxa-3-azabicyclo[4.1.0]heptane-3-carboxylate (324 mg, 1.63 mmol, 5 eq). The mixture was stirred at 70° C. for 16 hours. On completion, the mixture was concentrated in vacuo to give a residue. The residue was purified by prep-HPLC (column: Phenomenex luna C18 150*25 mm*10 um; mobile phase: [water(FA)-ACN]; B %: 52%-82%,15 min) to give tert-butyl (3S,4S)-4-[(17E)-5,15-dimethyl-21-tetrahydropyran-2-yl-7,11-dioxa-4,5,13,14,20,21-hexazapentacyclo[17.5.2.0^{2,6}.0^{12,16}0^{22,26}]hexacosa-1(25),2(6),3,12(16),14,17,19,22(26),23-nonaen-13-yl]-3-hydroxy-piperidine-1-carboxylate (15 mg, 0.022 mmol, 7% yield) as yellow solid: ¹H NMR (400 MHz, CDCl₃) δ=8.67-8.55 (m, 1H), 7.82-7.74 (m, 1H), 7.65-7.59 (m, 1H), 7.57-7.48 (m, 2H), 7.11-6.98 (m, 1H), 5.67 (d, J=10.0 Hz, 1H), 4.69-4.23 (m, 6H), 4.22-4.15 (m, 1H), 4.13-4.06 (m, 1H), 4.03-3.93 (m, 1H), 3.79 (d, J=7.6 Hz, 3H), 2.96-2.66 (m, 2H), 2.64-2.52 (m, 3H), 2.50 (s, 1H), 2.48-2.40 (m, 1H), 2.38-2.34 (m, 1H), 2.20-2.04 (m, 3H), 1.96-1.86 (m, 1H), 1.79 (t, J=8.8 Hz, 2H), 1.68-1.63 (m, 3H), 1.49 (s, 9H); LCMS: m/z 660.0 (M+1) and tert-butyl (3S,4S)-4-((E)-11,55-dimethyl-21-(tetrahydro-2H-pyran-2-yl)-11H,21H,51H-6,10-dioxa-2(5,3)-indazola-1(4,5),5(4,3)-dipyrazolacyclodecaphan-3-en-5l-yl)-3-hydroxypiperidine-1-carboxylate (15 mg, 0.022 mmol, 7% yield) as yellow solid: ¹H NMR (400 MHz, CDCl₃) δ=8.67-8.54 (m, 1H), 7.82-7.75 (m, 1H), 7.64-7.59 (m, 1H), 7.57-7.50 (m, 2H), 7.12-7.00 (m, 1H), 5.67 (d, J=10.0 Hz, 1H), 4.67-4.27 (m, 6H), 4.23-4.16 (m, 1H), 4.14-4.08 (m, 1H), 4.02-3.93 (m, 1H), 3.81-3.74 (m, 4H), 2.91-2.68 (m, 2H), 2.60-2.52 (m, 3H), 2.50 (s, 1H), 2.48-2.42 (m, 1H), 2.35 (d, J=4.4 Hz, 1H), 2.19-2.05 (m, 3H), 1.94-1.88 (m, 1H), 1.84-1.71 (m, 4H), 1.50-1.48 (m, 9H); LCMS: m/z 660.1 (M+1).

The remaining steps were performed in a similar manner to the final deprotection in General Method C to give racemic Ex. 80 and racemic Ex. 81.

### Preparation of (rac)-4-[(17E)-8,16-dimethyl-2,8,11,12-tetrahydro-10H,14H-3,5-etheno[1,5]dioxacyclopentadecino[10,11-c:15,14-c′:6,7-c′]tripyrazol-14-yl]-1-methylpiperidin-3-ol (Ex. 82)

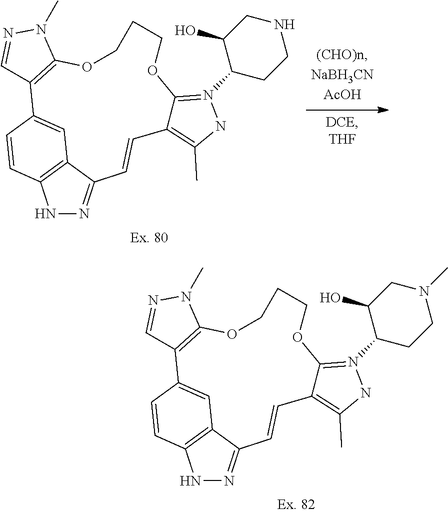

Step 1. To a solution of (rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]piperidin-3-ol (7 mg, 0.012 mmol, 1 eq, TFA) in DCE (1 mL) and THF (0.5 mL) was added AcOH (5.50 mg, 0.092 mmol, 6 eq) and PARAFORMALDEHYDE (30 mg). The mixture was stirred at 25° C. for 0.5 hour. Then NaBH(OAc)₃ (9.71 mg, 0.046 mmol, 3 eq) was added to the mixture and the mixture was stirred at 25° C. for 15.5 hours. On completion, the mixture was concentrated in vacuo to give a residue. The residue was purified by prep-HPLC (column: Phenomenex luna C18 150*25 mm*10 um; mobile phase: [water(FA)-ACN]; B %: 35%-65%,15 min) to give (rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-1-methylpiperidin-3-ol (4.16 mg, 0.008 mmol, 54% yield) as yellow solid.

Example 83 was synthesized from racemic Ex. 81 in a similar manner to the procedure used for Ex. 82.

### Preparation of (17E)-8,9,16-trimethyl-15-[2-(pyrrolidin-1-yl)ethyl]-2,8,9,11,12,15-hexahydro-3,5-etheno[1,4]dioxacyclopentadecino[11,10-c:15,14-c′:6,7-c″]tripyrazole (Ex. 84)

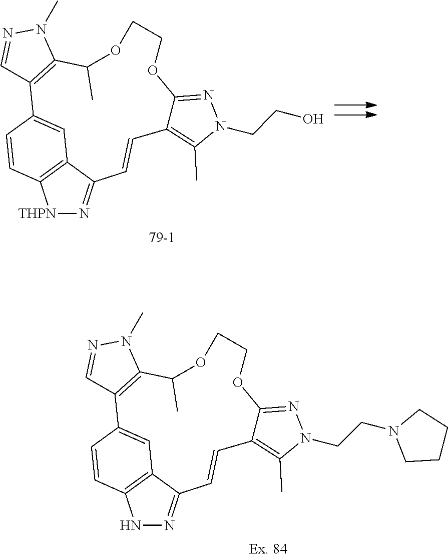

Example 84 was synthesized from 79-1 and appropriate amine following the procedures of General Method F.

### Preparation of (rac)-1,5-anhydr-3o-2,3-dideoxy-3-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-L-threo-pentitol (Ex. 85), (rac)-1,5-anhydro-2,4-dideoxy-2-[(17E)-8,16-dimethyl-2,8,11,12-tetrahydro-10H,14H-3,5-etheno[1,5]dioxacyclopentadecino[10,11-c:15,14-c′:6,7-c″ ]tripyrazol-14-yl]-L-threo-pentitol (Ex. 86), (rac)-1,5-anhydro-2,3-dideoxy-3-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-L-threo-pentitol (Ex. 87), and 1,5-anhydro-2,4-dideoxy-2-[(17E)-8,16-dimethyl-2,8,11,12-tetrahydro-10H,15H-3,5-etheno[1,5]dioxacyclopentadecino[10,11-c:15,14-c′:6,7-c″ ]tripyrazol-15-yl]-L-threo-pentitol

### (Ex. 88)

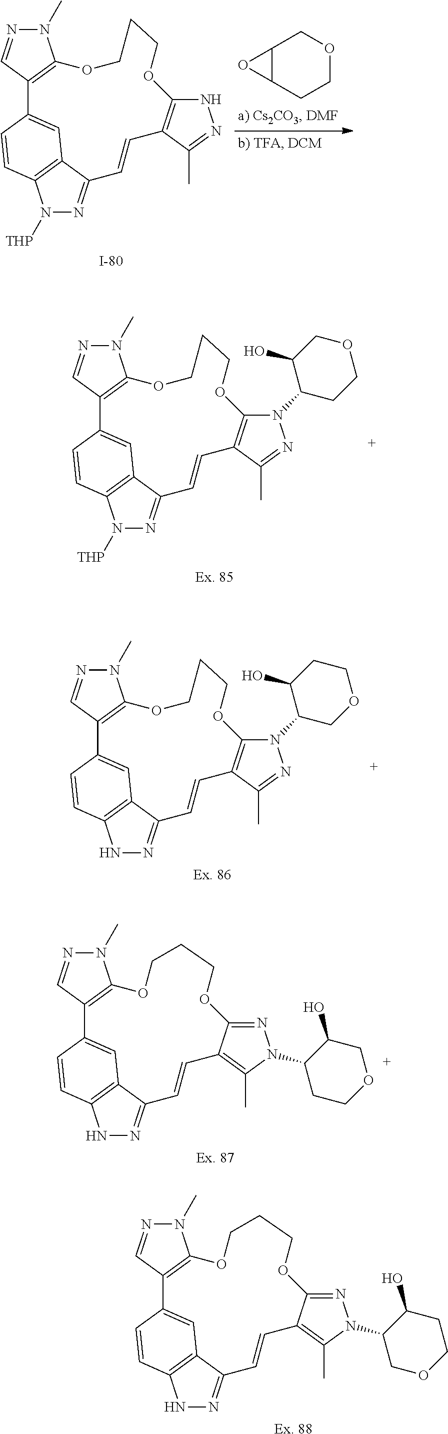

Steps 1 and 2 were performed in a similar manner to those of Example 80 to give arbitrarily assigned racemic Ex. 85, Ex. 86, Ex. 87, and Ex. 88.

### Preparation of 2-[3-(bromomethyl)-4-iodo-5-methyl-pyrazol-1-yl]acetate (I-89)

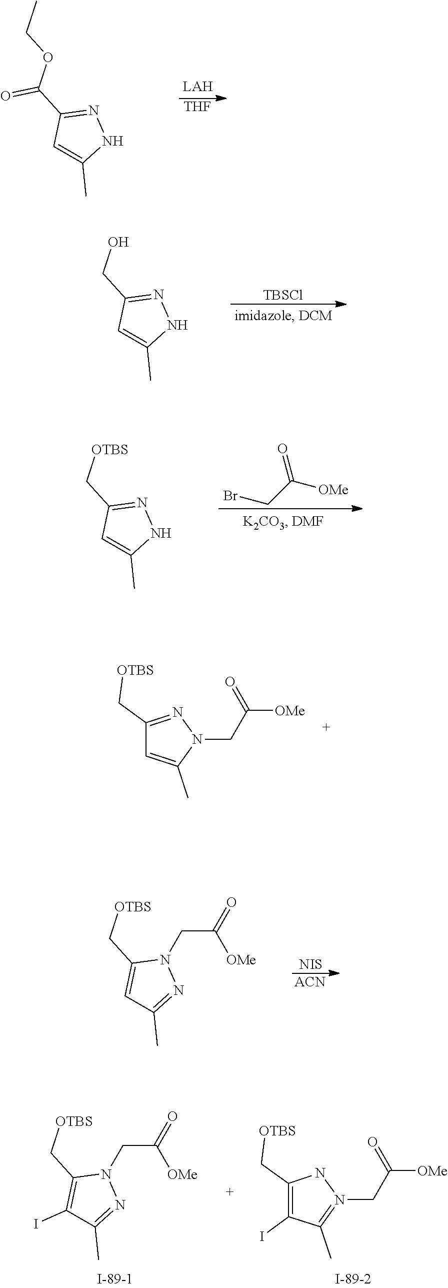

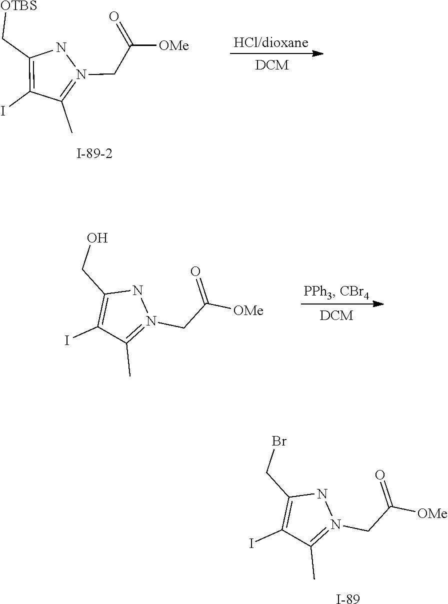

Step 1. To a mixture of ethyl 5-methyl-1H-pyrazole-3-carboxylate (10.0 g, 64.8 mmol, 1 eq) in THF (100 mL) was added LAH (2.95 g, 77.8 mmol, 1.2 eq), the reaction mixture was stirred at 0° C. for 2 hours. On completion, the mixture was slowly added to water (3 ml), saturated sodium hydroxide (3 ml) and water (9 ml) to quench. The reaction mixture was filtered and concentrated under reduced pressure to afford (5-methyl-111-pyrazol-3-yl)methanol (3.40 g, 30.3 mmol, 46% yield) as yellow solid. 1H NMR (400 MHz, DMSO-d₆) δ 5.89 (s, 1H), 4.36 (s, 2H), 2.16 (s, 3H).

Step 2. To a mixture of (5-methyl-1H-pyrazol-3-yl)methanol (25.0 g, 222 mmol, 1 eq) in DCM (300 mL) was added imidazole (30.3 g, 445 mmol, 2 eq) and TBSCl (50.4 g, 334 mmol, 40.9 mL, 1.5 eq) at 0° C., the reaction mixture was stirred at 25° C. for 12 hours. On completion, the residue was diluted with water (300 mL) and extracted with DCM (2×300 mL). The combined organic layers was dried over Na₂SO₄, filtered and concentrated in vacuo to afford tert-butyl-dimethyl-[(5-methyl-1H-pyrazol-3-yl)methoxy]silane (65.0 g, 90% yield) as yellow oil. ¹H NMR (400 MHz, DMSO-d₆) δ 12.34-12.09 (m, 1H), 5.90 (s, 1H), 4.55 (s, 2H), 2.17 (s, 3H), 0.86 (s, 9H), 0.05-0.02 (m, 6H).

Step 3. A mixture of methyl 2-bromoacetate (40.5 g, 265 mmol, 25.0 mL, 2 eq), tert-butyl-dimethyl-[(5-methyl-1H-pyrazol-3-yl)methoxy]silane (30.0 g, 132 mmol, 1 eq), K₂CO₃ (54.9 g, 397 mmol, 3 eq) in DMF (300 mL). The mixture was stirred at 80° C. for 16 hours. On completion, the reaction mixture was filtered and then the residue was diluted with water (500 mL) and extracted with EA (2×500 mL). The combined organic layers was dried over Na₂SO₄, filtered and concentrated in vacuo to give a residue. The residue was purified by column chromatography to afford methyl 2-[3-[[tert-butyl(dimethyl)silyl]oxymethyl]-5- methyl-pyrazol-1-yl]acetate and methyl 2-[5-[[tert-butyl (dimethyl)silyl]oxymethyl]-3- methyl-pyrazol-1-yl]acetate (21.2 g, 35.5 mmol, 52% yield) as yellow oil. ¹H NMR (400 MHz, DMSO-d₆) δ 5.98 (s, 111), 4.94 (s, 2H), 4.91 (s, 2H), 3.65 (s, 3H), 2.17 (s, 3H), 0.86 (s, 9H), 0.04 (s, 6H).

Step 4. To a mixture of methyl 2-[5-[[tert-butyl(dimethyl)silyl]oxymethyl]-3-methyl -pyrazol-1-yl]acetate and methyl 2-[3-[[tert-butyl (dimethyl) silyl]oxymethyl]-5-methyl-pyrazol-1-yl]acetate (21.2 g, 35.5 mmol, 1 eq) in ACN (200 mL) was added NIS (7.99 g, 35.5 mmol, 1 eq), the reaction mixture was stirred at 25° C. for 12 hours. On completion, the residue was diluted with water (200 mL) and extracted with EA (2×200 mL). The combined organic layers was dried over Na₂SO₄, filtered and concentrated in vacuo to give a residue. The residue was purified by column chromatography to afford methyl 2-[5-[[tert-butyl (dimethyl) silyl]oxymethyl]-4-iodo-3-methyl -pyrazol-1-yl]acetate (13.0 g, 30.6 mmol, 86% yield) yellow oil: ¹H NMR (400 MHz, DMSO-d₆) δ 5.09 (s, 2H), 4.48 (s, 2H), 3.68 (s, 3H), 2.20 (s, 3H), 0.87 (s, 9H), 0.06 (s, 6H). and methyl 2-[3-[[tert-butyl(dimethyl) silyl]oxymethyl]-4-iodo-5- methyl-pyrazol-1-yl]acetate (6.19 g, 14.5 mmol, 41% yield) as yellow solid: ¹H NMR (400 MHz, DMSO-d₆) δ 5.03 (s, 2H), 4.64 (s, 2H), 3.65 (s, 3H), 2.11 (s, 3H), 0.85 (s, 9H), 0.06 (s, 6H).

Step 5. To a mixture of methyl 2-[3-[[tert-butyl(dimethyl)silyl]oxymethyl]-4-iodo-5-methyl-pyrazol-1-yl]acetate (12.0 g, 28.2 mmol, 1 eq) in DCM (170 mL) was added HCl/dioxane (4 M, 20 mL, 2.83 eq). The reaction mixture was stirred at 25° C. for 12 hour. On completion, the reaction mixture was concentrated in vacuo. The residue was diluted with water (100 mL) and extracted with EA (2×100 mL). The combined organic layers was dried over Na₂SO₄, filtered and concentrated in vacuo to give a residue. The residue was purified by column chromatography afford to methyl 2-[3-(hydroxymethyl)-4-iodo-5-methyl-pyrazol-1-yl]acetate (7.9 g, 25.4 mmol, 90% yield) as white solid. ¹H NMR (400 MHz, DMSO-d6) δ 6.23 (s, 4H), 5.08 (s, 2H), 4.28 (s, 2H), 2.19 (s, 3H). LCMS: m/z 311.0 (M+1).

Step 6. To a mixture of methyl 2-[3-(hydroxymethyl)-4-iodo-5-methyl-pyrazol-1-yl]acetate (6.70 g, 21.6 mmol, 1 eq) in DCM (70 mL) was added CBr₄ (10.7 g, 32.4 mmol, 1.5 eq) and PPh₃ (8.50 g, 32.4 mmol, 1.5 eq) at 0° C. The reaction mixture was stirred at 25° C. for 2 hours. On completion, the residue was diluted with water (80 mL) and extracted with EA (2×90 mL). The combined organic layers were dried over Na₂SO₄, filtered and concentrated in vacuo to give a residue. The residue was purified by column chromatography to afford methyl 2-[3-(bromomethyl)-4-iodo-5-methyl-pyrazol-1-yl]acetate (9.60 g, crude) as white solid. ¹H NMR (400 MHz, DMSO-d6) δ 5.14 (s, 2H), 4.48 (s, 2H), 3.69 (s, 3H), 2.21 (s, 3H). LCMS: m/z 372.9 (M+1).

### General Method M: Preparation of (10S,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine (Ex. 89)

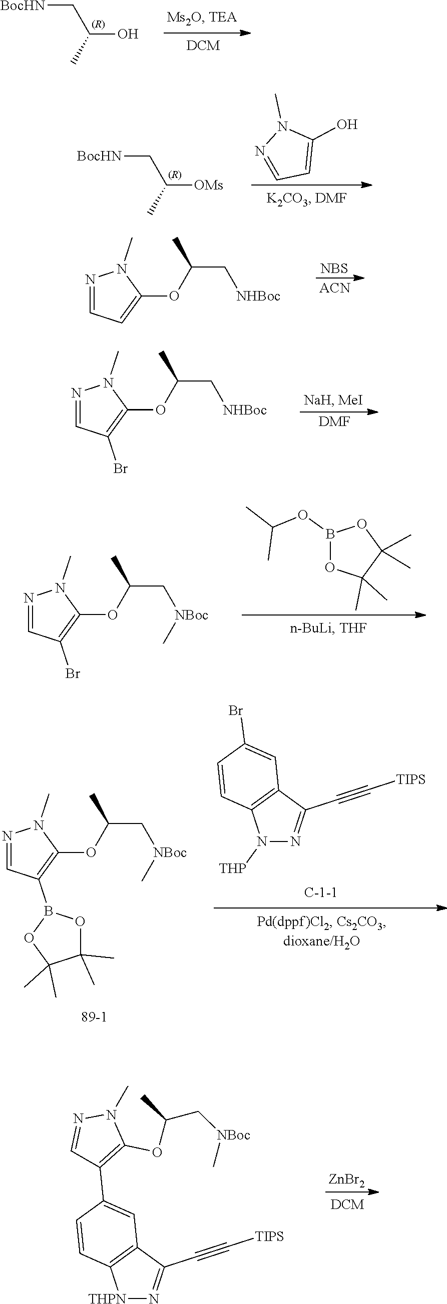

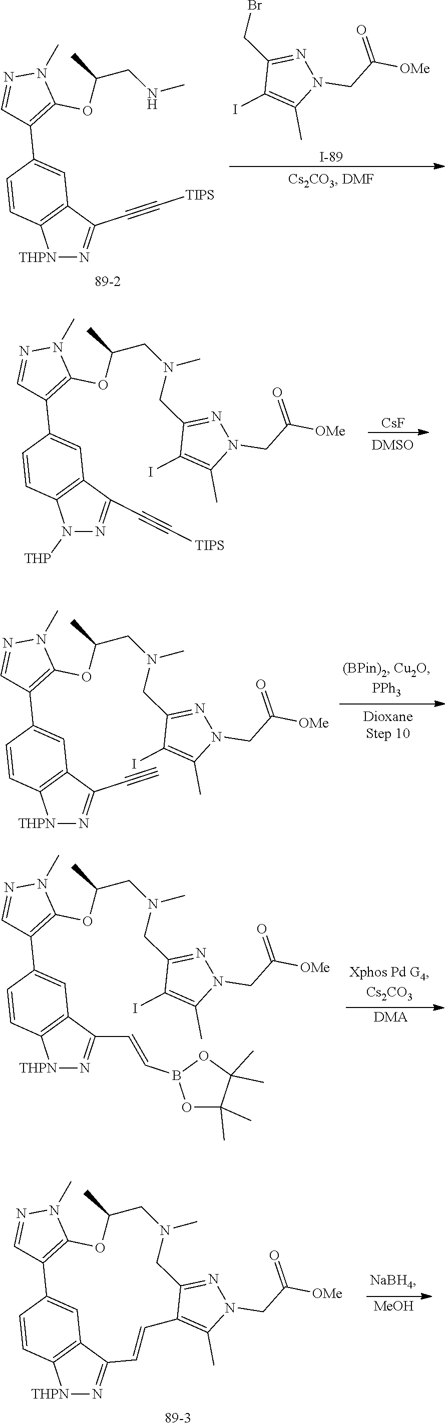

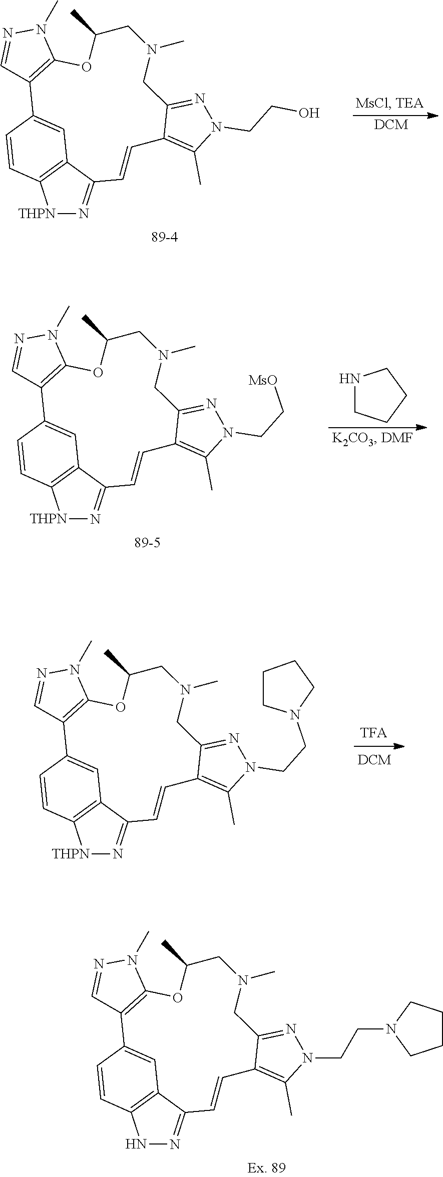

Step 1. To a mixture of tert-butyl N-[(2R)-2-hydroxypropyl]carbamate (30.0 g, 171 mmol, 1 eq) in DCM (250 mL) was added TEA (51.9 g, 513 mmol, 71.4 mL, 3 eq) and MsCl (23.5 g, 205 mmol, 15.9 mL, 1.2 eq) at 0° C. The reaction mixture was stirred at 25° C. for 2 hours. On completion, the reaction mixture was diluted with water (1000 mL) and extracted with EA (2×800 mL). The combined organic layers were dried over Na₂SO₄, filtered and concentrated in vacuo to afford to [(1R)-2-(tert-butoxycarbonylamino)-1-methyl-ethyl]methanesulfonate (29.0 g, 114 mmol, 66% yield) as yellow oil.

Step 2. To a mixture of [(1R)-2-(tert-butoxycarbonylamino)-1-methyl-ethyl]methanesulfonate (29.0 g, 114 mmol, 1 eq) and 2-methylpyrazol-3-ol (13.4 g, 137 mmol, 1.2 eq) in DMF (200 mL) was added K₂CO₃ (47.4 g, 343 mmol, 3 eq). The reaction mixture was stirred at 80° C. for 12 hours. On completion, the residue was diluted with water (300 mL) and extracted with EA (2×300 mL). The combined organic layers were dried over Na₂SO₄, filtered and concentrated in vacuo afford to tert-butyl N-[(2S)-2-(2-methylpyrazol-3-yl)oxypropyl]carbamate (29.0 g, 113 mmol, 99% yield) as yellow oil. ¹H NMR (400 MHz, DMSO-d₆) δ 8.00 (s, 2H), 7.22 (d, J=2.0 Hz, 1H), 4.32-4.23 (m, 1H), 3.56 (s, 3H), 3.27-3.14 (m, 2H), 1.41 (s, 12H); LCMS: m/z 256.3 (M+1).

Step 3. To a mixture of tert-butyl N-[(2S)-2-(2-methylpyrazol-3-yl)oxypropyl]carbamate (29.0 g, 113 mmol, 1 eq) in ACN (300 mL) was added NBS (22.2 g, 124 mmol, 1.1 eq) at 0° C. The reaction mixture was stirred at 25° C. for 2 hours. On completion, the residue was diluted with water (200 mL) and extracted with EA (2×150 mL). The combined organic layers was dried over Na₂SO₄, filtered and concentrated in vacuo to give a residue. The residue was purified by column chromatography afford tert-butyl N—[(2S)-2-(4-bromo-2-methyl-pyrazol-3-yl)oxypropyl]carbamate (15.4 g, 46.0 mmol, 40% yield) as yellow oil. ¹H NMR (400 MHz, DMSO-d₆) δ 7.37 (s, 1H), 7.09 (t, J=5.6 Hz, 1H), 4.58-4.49 (m, 1H), 3.61 (s, 3H), 3.19 (t, J=6.0 Hz, 2H), 1.37 (s, 9H), 1.22 (d, J=6.2 Hz, 3H).

Step 4. To a mixture of tert-butyl N-[(2S)-2-(4-bromo-2-methyl-pyrazol-3-yl)oxypropyl]carbamate (15.4 g, 46.0 mmol, 1 eq) in DMF (150 mL) was added NaH (3.69 g, 92.1 mmol, 60% purity, 2 eq) at 0° C. The reaction mixture was stirred at 0° C. for 0.5 hour. Then MeI (7.85 g, 55.29 mmol, 3.44 mL, 1.2 eq) was added. The reaction mixture was stirred at 25° C. for 12 hours. On completion, the residue was diluted with water (150 mL) and extracted with EA (2×200 mL). The combined organic layers was dried over Na₂SO₄, filtered and concentrated in vacuo to give a residue. The residue was purified by column chromatography afford tert-butyl N-[(2S)-2-(4-bromo-2-methyl-pyrazol-3-yl)oxypropyl]-N-methyl-carbamate (11.0 g, 31.59 mmol, 69% yield) as yellow oil. ¹H NMR (400 MHz, DMSO-d₆) δ 7.45-7.33 (m, 1H), 4.68 (brs, 1H), 3.61 (s, 3H), 3.43 (s, 2H), 2.88 (s, 3H), 1.37 (s, 9H), 1.22-1.19 (m, 3H).

Step 5 was conducted in a similar manner to step 4 in synthesis of B-2-4 to afford tert-butyl N-methyl-N-[(2S)-2-[2-methyl-4-(4,4,5,5-tetramethyl-1,3,2-dioxaborolan-2-yl)pyrazol-3-yl]oxypropyl]carbamate (6.00 g, 15.1 mmol, 48% yield) as yellow oil. ¹H NMR (400 MHz, DMSO-d₆) δ 7.37 (s, 1H), 5.14-4.84 (m, 1H), 3.54 (s, 3H), 3.48-3.37 (m, 2H), 2.88-2.78 (m, 3H), 1.37 (s, 9H), 1.17-1.15 (m, 3H), 1.07 (s, 12H); LCMS: m/z 396.2 (M+1).

Step 6 was conducted in a similar manner to step 1 in General Method C.

Step 7. To a mixture of tert-butyl N-methyl-N-[(2S)-2-[2-methyl-4-[1-tetrahydropyran-2-yl-3-(2-triisopropylsilylethynyl) indazol-5-yl]pyrazol-3-yl]oxypropyl]carbamate (4.70 g, 7.23 mmol, 1 eq) in DCM (50 mL) was added ZnBr₂ (8.14 g, 36.1 mmol, 5 eq). The reaction mixture was stirred at 25° C. for 3 hours. On completion, the residue was diluted with water (50 mL) and extracted with EA (2×50 mL). The combined organic layers was dried over Na₂SO₄, filtered and concentrated in vacuo to afford (2S)—N-methyl-2-[2-methyl-4-[1-tetrahydropyran-2-yl-3-(2-triisopropylsilylethynyl) indazol-5-yl]pyrazol-3-yl]oxy-propan-1-amine (3.00 g, 5.46 mmol, 75% yield) as yellow solid. ¹H NMR (400 MHz, DMSO-d₆) δ 7.85-7.81 (m, 1H), 7.75 (s, 1H), 7.72-7.65 (m, 2H), 5.93-5.86 (m, 1H), 4.29-4.20 (m, 1H), 3.97-3.87 (m, 2H), 3.79-3.74 (m, 1H), 3.73 (s, 3H), 3.49-3.39 (m, 1H), 2.95-2.88 (m, 1H), 2.86-2.80 (m, 1H), 2.39-2.34 (m, 3H), 2.04-1.96 (m, 2H), 1.81-1.70 (m, 1H), 1.59 (s, 2H), 1.18-1.13 (m, 21H), 1.07 (s, 3H); LCMS: m/z 550.7 (M+1).

Step 8. To a mixture of (2S)—N-methyl-2-[2-methyl-4-[1-tetrahydropyran-2-yl-3-(2-triisopropylsilylethynyl) indazol-5-yl]pyrazol-3-yl]oxy-propan-1-amine (2.90 g, 5.27 mmol, 1 eq) and methyl 2-[3-(bromomethyl)-4-iodo-5-methyl-pyrazol-1-yl]acetate (3.93 g, 10.5 mmol, 2 eq) in DMF (30 mL) was added Cs₂CO₃ (5.16 g, 15.8 mmol, 3 eq). The reaction mixture was stirred at 25° C. for 4 hours. On completion, the residue was diluted with water (60 mL) and extracted with EA (2×60 mL). The combined organic layers was dried over Na₂SO₄, filtered and concentrated in vacuo to afford methyl 2-[4-iodo-5-methyl-3-[[methyl-[(2S)-2-[2-methyl-4-[1-tetrahydropyran-2-yl-3-(2-triisopropylsilylethynyl) indazol-5-yl]pyrazol-3-yl]oxypropyl]amino]methyl]pyrazol-1-yl]acetate (4.00 g, 4.75 mmol, 90% yield) as yellow oil. LCMS: m/z 842.6 (M+1).

Steps 9 and 10 were performed in a similar manner to step 3 and 4 of General Method H.

Step 11. To a mixture of methyl 2-[4-iodo-5-methyl-3-[[methyl-[(2S)-2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxypropyl]amino]methyl]pyrazol-1-yl]acetate (500 mg, 0.614 mmol, 1 eq) in DMA (4 mL) was added Cs₂CO₃ (600 mg, 1.84 mmol, 3 eq) and Xphos Pd G₄ (52.8 mg, 0.061 mmol, 0.1 eq). The reaction mixture was stirred at 90° C. for 1 hour.

On completion, the residue was diluted with water (30 mL) and extracted with EA (2×30 mL). The combined organic layers was dried over Na₂SO₄, filtered and the filtrate was concentrated in vacuo to give a residue. The residue was purified by column chromatography afford to methyl 2-[(8S,17E)-5,8,10,15-tetramethyl-21-tetrahydropyran-2-yl-7-oxa-4,5,10,13, 14,20,21-heptazapentacyclo|17.5.2.0^{2,6}.0^{12,16}0.0^{22,26}|hexacosa-1(25),2(6),3,12,15,17,19, 22(26), 23-nonaen-14-yl]acetate (250 mg, 0.446 mmol, 72% yield) as yellow oil. LCMS: m/z 842.6 (M+1).

Step 12 was performed in a similar manner to step 1 of General Method D.

The remaining steps were performed in a similar manner to General Method F to give Ex. 89 (32.3 mg) as yellow solid.

### Preparation of (10S,17E)-8,10,12,16-tetramethyl-15-[2-(4-methylpiperazin-1-yl)ethyl]-2,10,11,12,13,15-hexahydro-8H-3,5-ethenotripyrazolo[3,4-f:3′,4′-j:4″,3″-n][1,4]oxazacyclopentadecine (Ex. 90)

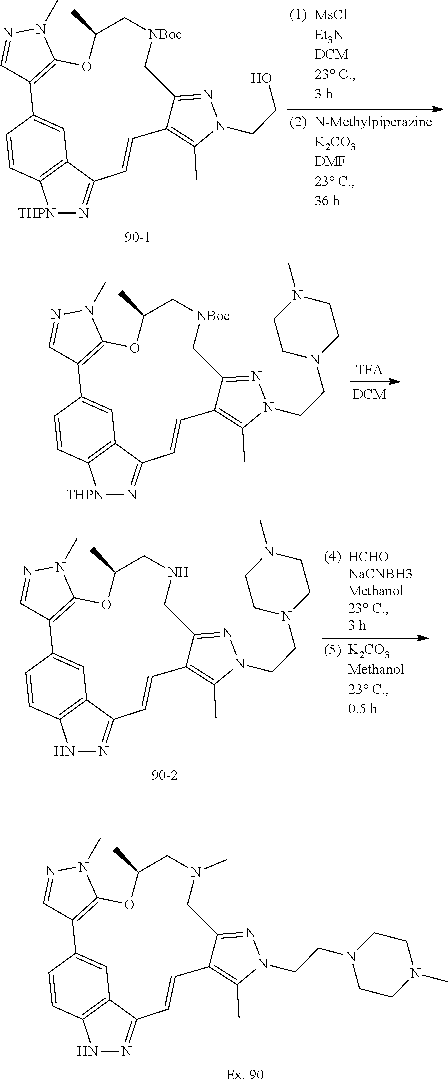

90-1 was an intermediate in the synthesis of Ex. 49.

90-1 was converted to 90-2 in a similar manner to General Method F.

Step 4. To a solution of 90-2 (0.7 mg, 0.0014 mmol) in Methanol (0.1 mL) was added Formaldehyde, 37% w/w aq. soln., (61.14 μg, 0.002 mmol), followed by Sodium cyanoborohydride (102.37 μg, 0.002 mmol). The mixture was stirred at room temperature for 3 h. The reaction mixture was then diluted with water (0.5 mL), extracted with ethyl acetate (3×1 mL), washed with brine and dried over sodium sulfate. The volatiles were evaporated in vacuo and the crude residue was taken up in methanol (60 μL) and to this mixture was added Potassium carbonate (562 μg, 0.004 mmol), and the mixture was stirred for 30 minutes. The reaction was then diluted with water (0.5 mL), extracted with DCM (3×1 mL) and the combined organic layers were evaporated in vacuo. The crude residue was purified by preparative HPLC (C18, 21 mm×100 mm, MeCN in water with 0.035% TFA) to obtain Ex. 90.

### Preparation of 2-[3-(4-bromo-2-methyl-pyrazol-3-yl) oxypropoxy]-4-methoxy-pyrazolo[1,5-a]pyrazine (I-91)

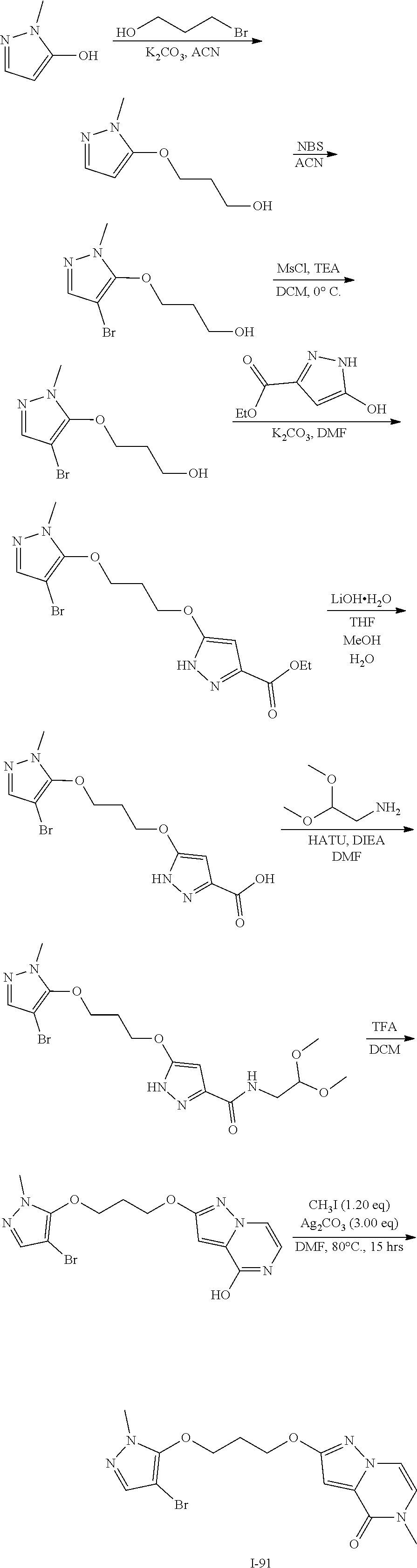

Step 1. To a solution of 2-methylpyrazol-3-ol (20.0 g, 203 mmol, 1 eq) in ACN (200 mL) was added K₂CO₃ (84.5 g, 611 mmol, 3 eq) and 3-bromopropan-1-ol (28.3 g, 203 mmol, 18.4 mL, 1 eq). The mixture was stirred at 25° C. for 16 hr. On completion, the reaction mixture was concentrated under reduced pressure to remove ACN. The residue was purified by column chromatography (SiO₂, DCM/MeOH=10/1 to 1/1) to give 3-(2-methylpyrazol-3-yl)oxypropan-1-ol (27.3 g, 175 mmol, 85% yield) as a colorless oil. ¹H NMR (400 MHz, CDCl₃) δ=7.28-7.22 (m, 1H), 5.48 (d, J=1.6 Hz, 1H), 4.26-4.05 (m, 2H), 3.86-3.70 (m, 2H), 3.65-3.52 (m, 3H), 2.13-1.95 (m, 2H). LCMS: m/z 156.9 (M+1)

Step 2. To a solution of 3-(2-methylpyrazol-3-yl)oxypropan-1-ol (27.0 g, 172 mmol, 1 eq) in ACN (180 mL) was added NBS (30.7 g, 172 mmol, 1 eq). The mixture was stirred at 20° C. for 1 hr. On completion, the reaction mixture was quenched by addition Na₂SO₃ (200 mL) at 0° C., and then diluted with H₂O (250 mL). The mixture was separated and extracted with DCM 600 mL (300 mL*2). The combined organic layers were washed with sat NaCl (500 mL), dried over Na₂SO₄, filtered and filtrate was concentrated under reduced pressure to give a residue. The residue was purified by column chromatography (SiO₂, Petroleum ether/Ethyl acetate=5/1 to 3/1) to give 3-(4-bromo-2-methyl-pyrazol-3-yl)oxypropan-1-ol (21.1 g, 89.7 mmol, 52% yield) as a yellow oil. LCMS: m/z 234.6

Step 3. To a solution of 3-(4-bromo-2-methyl-pyrazol-3-yl)oxypropan-1-ol (21.1 g, 89.7 mmol, 1 eq) in DCM (200 mL) was added TEA (27.2 g, 269 mmol, 37.5 mL, 3 eq) and MsCl (20.5 g, 179 mmol, 13.9 mL, 2 eq) at 0° C. The mixture was stirred at 25° C. for 1 hr. On completion, the reaction mixture was quenched by addition NaHCO₃50 mL at 0° C., and then diluted with H₂O (200 mL) and extracted with DCM (200 mL*3). The combined organic layers were washed with H₂O (100 mL *3), dried over Na₂SO₄, filtered and concentrated under reduced pressure to give a residue to give 3-(4-bromo-2-methyl-pyrazol-3-yl) oxypropyl methanesulfonate (28.0 g, 89.4 mmol, 100% yield) as brown oil.

Step 4. To a solution of 3-(4-bromo-2-methyl-pyrazol-3-yl)oxypropyl methanesulfonate (28.0 g, 89.4 mmol, 1 eq) in DMF (120 mL) was added K₂CO₃ (37.1 g, 268 mmol, 3 eq) and ethyl 5-hydroxy-1H-pyrazole-3-carboxylate (13.3 g, 84.9 mmol, 0.95 eq) at 20° C. The mixture was stirred at 60° C. for 16 hr. On completion, the reaction mixture was diluted with H₂O (400 mL) and extracted with EtOAc (100 mL*3). The combined organic layers were washed with sat. NaCl (250 mL), dried over Na₂SO₄, filtered and filtrate was concentrated under reduced pressure to give a residue. The residue was purified by column chromatography (SiO₂, Petroleum ether/Ethyl acetate=10/1 to 1/1) to give ethyl 5-[3-(4-bromo-2-methyl-pyrazol-3-yl)oxypropoxy]-1H-pyrazole-3-carboxylate (17.3 g, 46.4 mmol, 52% yield) as a white solid.

Step 5. To a solution of ethyl 5-[3-(4-bromo-2-methyl-pyrazol-3-yl)oxypropoxy]-1H-pyrazole-3-carboxylate (14.0 g, 37.5 mmol, 1 eq) in THF (20 mL), MeOH (20 mL), H₂O (10 mL) and LiOH·H₂O (1.57 g, 37.5 mmol, 1 eq). The mixture was stirred at 20° C. for 15 hr. On completion, the reaction mixture was diluted with H₂O (50 mL) and then 0.5 M aq. HCl solution (50 ml) was added until the pH=5 and extracted with EtOAc (40 mL*3). The combined organic layers were washed with sat. NaCl (100 mL), dried over Na₂SO₄, filtered and concentrated under reduced pressure to give a residue. The crude product was triturated with EtOAc (50 mL). Then the mixture was filtered to give 5-[3-(4-bromo-2-methyl-pyrazol-3-yl)oxypropoxy]-1H-pyrazole-3-carboxylic acid (8.00 g, 23.2 mmol, 62% yield, crude purity) as a white solid. LCMS: m/z 346.5 (M+1)

Step 6. To a solution of 5-[3-(4-bromo-2-methyl-pyrazol-3-yl)oxypropoxy]-1H-pyrazole-3-carboxylic acid (8.00 g, 23.2 mmol, 1 eq) in DMF (80 mL) was added 2,2-dimethoxyethanamine (3.66 g, 34.7 mmol, 3.79 mL, 1.5 eq), HATU (13.2 g, 34.8 mmol, 1.5 eq) and DIEA (8.99 g, 69.5 mmol, 12.1 mL, 3 eq). The mixture was stirred at 20° C. for 2 hr. On completion, the reaction mixture was diluted with H₂O (200 mL) and extracted with EtOAc (50 mL*5). The combined organic layers were washed with aq NaCl solution (500 mL), dried over Na₂SO₄, filtered and concentrated under reduced pressure to give a residue. The residue was purified by column chromatography (SiO₂, Petroleum ether/Ethyl acetate=10/1 to 1/1) to give 5-[3-(4-bromo-2-methyl-pyrazol-3-yl)oxypropoxy]-N-(2,2-dimethoxyethyl)-1H-pyrazole-3-carboxamide (8.80 g, 20.4 mmol, 87% yield) as a white oil. ¹H NMR (400 MHz, CDCl₃) δ=7.29 (s, 1H), 6.64 (s, 1H), 6.02 (s, 1H), 4.48 (t, J=5.2 Hz, 1H), 4.44 (t, J=6.4 Hz, 2H), 4.37 (t, J=6.4 Hz, 2H), 3.66 (s, 3H), 3.58 (t, J=5.6 Hz, 2H), 3.44 (s, 6H), 2.31-2.18 (m, 2H). LCMS: m/z 455.5 (M+Na)

Step 7. To a solution of 5-[3-(4-bromo-2-methyl-pyrazol-3-yl)oxypropoxy]-N-(2,2-dimethoxyethyl)-1H-pyrazole-3- carboxamide (6.00 g, 13.9 mmol, 1 eq) in DCM (60 ml) was added TFA (12 mL). The mixture was stirred at 20° C. for 2 hrs. On completion, the reaction mixture was concentrated under reduced pressure to remove DCM and give a residue. The residue was purified by column chromatography (SiO₂, Petroleum ether/Ethyl acetate=5/1 to 1/1) to give 2-[3-(4-bromo-2-methyl-pyrazol-3-yl)oxypropoxy]pyrazolo[1,5-a]pyrazin-4-ol (4.61 g, 12.5 mmol, 90% yield) as a white solid. LCMS: m/z 367.9 (M+1)

Step 8. At 20° C., a solution of 2-[3-(4-bromo-2-methyl-pyrazol-3-yl)oxypropoxy]pyrazolo[1,5-a]pyrazin-4-ol (4.60 g, 12.5 mmol, 1 eq) in DMF (30 mL) was added CH₃I (2.13 g, 14.9 mmol, 1.2 eq) and Ag₂CO₃ (10.3 g, 37.5 mmol, 3 eq). The mixture was stirred 80° C. for 8 h. On completion, the reaction mixture was diluted with H₂O (100 ml) and extracted with EtOAc (50 mL*3). The organic phase was separated, washed with H₂O (300 mL), dried over Na₂SO₄, filtered and concentrated under reduced pressure to give a residue. The residue was purified by column chromatography (SiO₂, Petroleum ether/Ethyl acetate=10/1 to 1/1) to give 2-|3-(4-bromo-2-methyl-pyrazol-3-yl) oxypropoxy|-4-methoxy-pyrazolo[1,5-a]pyrazine (3.00 g, 7.85 mmol, 62% yield) a yellow solid. LCMS: m/z 381.9 (M+1)

### Preparation of (20E)-8,18-dimethyl-2,8,11,12-tetrahydro-10H-3,5-ethenodipyrazolo[3″,4″:10′,11′;4′″,3′″:14′,15′][1,5]dioxacyclopentadecino[6′,7′:3,4]pyrazolo[1, 5-a]pyrazin-19(18H)-one (Ex. 91)

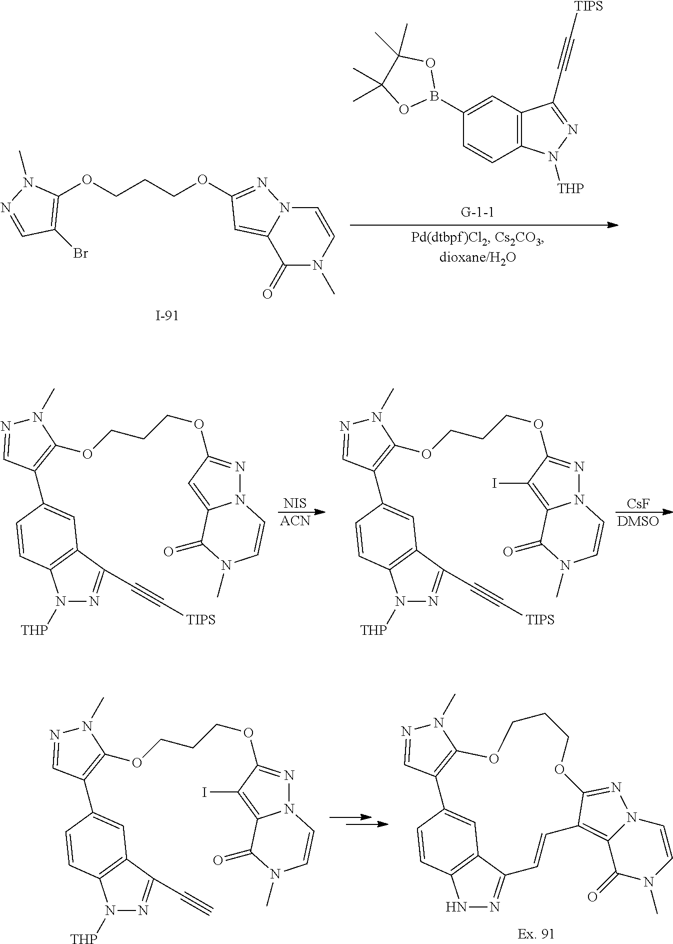

Step 1. A mixture of 2-[3-(4-bromo-2-methyl-pyrazol-3-yl)oxypropoxy]-5-methyl-pyrazolo[1,5-a]pyrazin-4-one (3.00 g, 7.85 mmol, 1 eq), triisopropyl-[2-[1-tetrahydropyran-2-yl-5-(4,4,5,5-tetramethyl-1,3,2-dioxaborolan-2-yl) indazol-3- yl]ethynyl]silane (4.79 g, 9.42 mmol, 1.2 eq), Cs₂CO₃ (7.67 g, 23.5 mmol, 3 eq) and ditertbutyl (cyclopentyl)phosphane;dichloropalladium;iron (511 mg, 0.785 mmol, 0.1 eq) in H₂O (12 mL) and dioxane (30 mL) was degassed and purged with N2 for 3 times, and then the mixture was stirred at 80° C. for 2 h under N₂ atmosphere. On completion, the mixture was diluted with H₂O (200 mL) and extracted with EtOAc (50 mL*3). The combined organic layers were washed with sat. NaCl solution (200 mL), dried over Na₂SO₄, filtered and concentrated under reduced pressure to give a residue. The residue was purified by column chromatography (SiO₂, Petroleum ether/Ethyl acetate=10/1 to 2/1) to give 5-methyl-2-[3-[2-methyl-4-[1-tetrahydropyran-2-yl-3-(2-triisopropylsilylethynyl) indazol-5-yl]pyrazol-3-yl]oxypropoxy]pyrazolo [1,5-a]pyrazin-4-one (4.40 g, 6.43 mmol, 81% yield) as a white solid. ¹H NMR (400 MHz, CDCl₃) δ=7.77 (s, 1H), 7.53-7.47 (m, 3H), 7.15 (d, J=6.0 Hz, 1H), 6.42 (d, J=6.0 Hz, 1H), 6.28 (s, 1H), 5.64 (dd, J=2.8, 9.2 Hz, 1H), 4.30 (t, J=6.0 Hz, 2H), 4.12-3.94 (m, 4H), 3.68 (s, 5H), 3.44 (s, 3H), 2.46 (s, 1H), 2.15-2.04 (m, 3H), 2.03-1.94 (m, 2H), 1.85 (s, 3H), 1.77-1.65 (m, 3H), 1.20 (s, 4H), 1.12-1.10 (m, 18H). LCMS: m/z 683.9 (M+1)

Step 2. To a solution of 5-methyl-2-[3-[2-methyl-4-[1-tetrahydropyran-2-yl-3-(2-triisopropylsilylethynyl) indazol-5-yl]pyrazol-3-yl]oxypropoxy]pyrazolo[1,5-a]pyrazin-4-one (4.40 g, 6.43 mmol, 1 eq) in ACN (70 mL) was added NIS (2.17 g, 9.65 mmol, 1.5 eq) at 0° C. The mixture was stirred at 0° C. for 2 h. On completion, the reaction mixture was quenched by addition sat. Na₂SO₃ solution (50 mL) at 0° C., then diluted with H₂O (20 mL) and extracted with EA (30 mL*2). The combined organic layers were washed with sat. NaCl solution (50 mL) and dried over Na₂SO₄, filtered and concentrated under reduced pressure to give a residue. The residue was purified by column chromatography (SiO₂, Petroleum ether/Ethyl acetate=10/1 to 2/1) to give 3-iodo-5-methyl-2-[3-[2-methyl-4-[1-tetrahydropyran-2-yl-3-(2-triisopropylsilylethynyl) indazol-5-yl]pyrazol-3-yl]oxypropoxy]pyrazolo[1,5-a]pyrazin-4-one (3.80 g, 4.69 mmol, 72% yield) as a yellow solid. ¹H NMR (400 MHz, CDCl₃) δ=7.72 (s, 1H), 7.67 (s, 1H), 7.58 (d, J=8.4 Hz, 1H), 7.41 (d, J=8.4 Hz, 1H), 7.13 (d, J=6.0 Hz, 1H), 6.45 (d, J=6.0 Hz, 1H), 5.66 (dd, J=2.4, 9.2 Hz, 1H), 4.38 (t, J=6.0 Hz, 2H), 4.12 (t, J=6.0 Hz, 2H), 4.05 (q, J=7.2 Hz, 1H), 4.01-3.88 (m, 4H), 3.76-3.61 (m, 1H), 3.41 (s, 3H), 2.54-2.38 (m, 2H), 2.20-1.90 (m, 13H), 1.80-1.56 (m, 5H), 1.20-1.17 (m, 4H), 1.11 (s, 14H). LCMS: m/z 810.3 (M+1)

Step 3. To a solution of 3-iodo-5-methyl-2-[3-[2-methyl-4-[1-tetrahydropyran-2-yl-3-(2-triisopropylsilylethynyl) indazol-5-yl]pyrazol-3-yl]oxypropoxy]pyrazolo[1,5-a]pyrazine-4-one (3.80 g, 4.69 mmol, 1 eq) in DMSO (20 mL) was added CsF (712 mg, 4.69 mmol, 1 eq). The mixture was stirred at 20° C. for 1 h. On completion, the reaction mixture was quenched by addition sat. NH₄Cl solution (10 mL), then diluted with H₂O (50 mL) and extracted with EA (30 mL*4). The combined organic layers were washed with H₂O (200 mL), dried over Na₂SO₄, filtered and concentrated under reduced pressure to give a residue. The residue was purified by column chromatography (SiO₂, Petroleum ether/Ethyl acetate=10/1 to 1/1) to give 2-[3-[4-(3-ethynyl-1-tetrahydropyran-2-yl-indazol-5-yl)-2-methyl-pyrazol-3-yl]oxypropoxy]-3-iodo-5-methyl-pyrazolo[1,5-a]pyrazin-4-one (2.79 g, 4.27 mmol, 90% yield) a yellow solid. ¹H NMR (400 MHz, CDCl₃) δ=7.77 (s, 1H), 7.62 (s, 1H), 7.57 (d, J=8.8 Hz, 1H), 7.51-7.42 (m, 1H), 7.13 (d, J=6.0 Hz, 1H), 6.44 (d, J=6.0 Hz, 1H), 5.66 (dd, J=2.8, 8.9 Hz, 1H), 5.23 (s, 1H), 4.41 (t, J=6.0 Hz, 2H), 4.10 (t, J=6.0 Hz, 2H), 4.00-3.89 (m, 1H), 3.83 (s, 3H), 3.75-3.63 (m, 1H), 3.50-3.37 (m, 3H), 3.32 (s, 1H), 2.55 (s, 1H), 2.50-2.39 (m, 2H), 2.21-2.10 (m, 3H), 1.79-1.56 (m, 3H). LCMS: m/z 653.9 (M+1)

The remaining steps were performed in a manner similar to those described in General Method L to give Ex. 91.

### Preparation of (10R,16E)-3-methyl-13-[(4-methylpiperazin-1-yl)methyl]-3,5,6,9,10,19-hexahydro-8H-20,22-etheno-7,10-methanodipyrazolo[3,4-i:4′,3′-m][1,8,5]benzodioxazacyclooctadecin-25-one (Ex. 92)

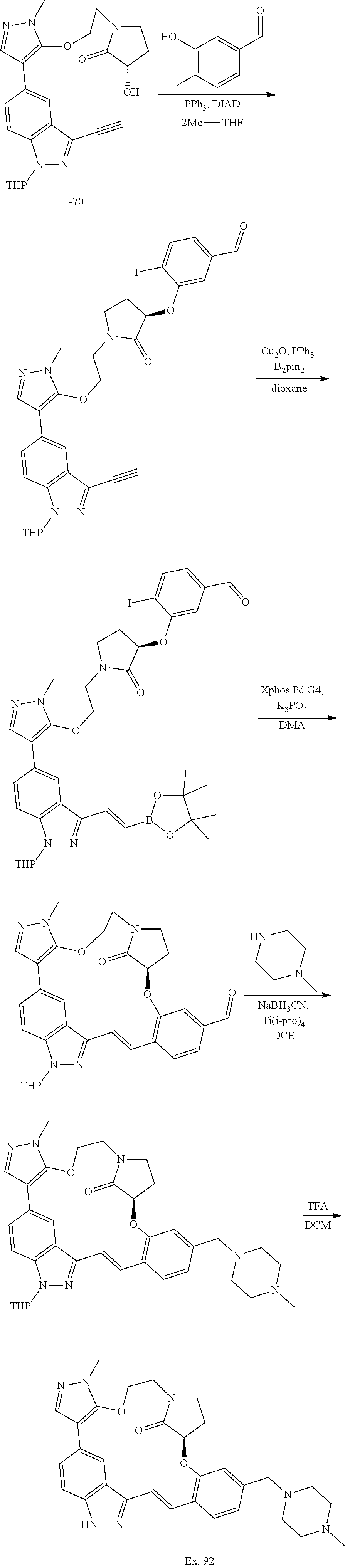

Step 1 through 3 were performed in a similar manner to those described in General Method L from I-70.

Step 4. To a solution of (13R,21E)-5-methyl-31-oxo-25-tetrahydropyran-2-yl-7,14-dioxa-4,5,10,24,25-pentazahexacyclo[21.5.2.110,13.02,6.015,20.026,30]hentriaconta-1(29)

,2(6),3,15(20),16,18,21,23,26(30),27-decaene-17-carbaldehyde (50 mg, 0.090 mmol, 1 eq) and 1-methylpiperazine (36.2 mg, 0.361 mmol, 40.1 uL, 4 eq) in DCE (5 mL) was added Ti(i-PrO)₄ (51.3 mg, 0.181 mmol, 53.3 uL, 2 eq). The mixture was stirred at 20° C. for 1 h, then NaBH₃CN (17.0 mg, 0.271 mmol, 3 eq) was added and the mixture was stirred at 20° C. for 2 h. On completion, the mixture was filtered and the filtrate was concentrated in vacuum to give crude. The residue was purified by combi flash (12 g silica gel column, THF in PE from 0-100%) to give (13R,21E)-5-methyl-17-[(4-methylpiperazin-1-yl)methyl]-25-tetrahydropyran-2-yl-7,14-dioxa-4,5,10,24,25-pentazahexacyclo[21.5.2.110,13.02,6.015,20.026,30]hentriaconta-1(29),2(6),3,15(20), 16,18,21,23,26(30),27-decaen-31-one (20 mg, 0.031 mmol, 35% yield) as a brown oil. LCMS: m/z 638.2 (M+1).

Step 5 was performed in a similar manner to the last step in General Method C to give Ex. 92.

Example 93 was made following the procedures of General Method M.

Example 94 was made in a similar manner to Ex. 92.

### Preparation of 4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol (Ex. 95)

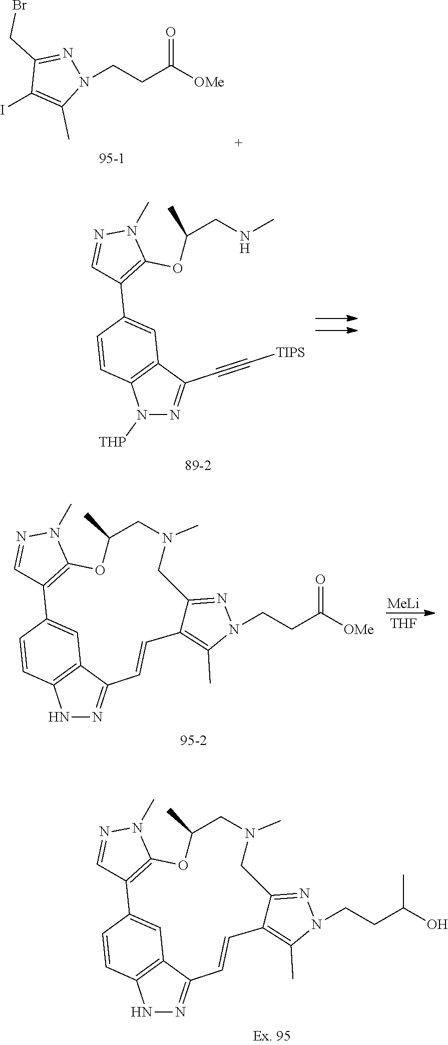

95-1 was synthesized in the same manner as I-89 starting with methyl 3-bromopropanoate.

95-1 and 89-2 were combined following the procedures described in General Method M to afford 95-2.

Step 2. A mixture of methyl 3-[(8S,17E)-5,8,10,15-tetramethyl-7-oxa-4,5,10,13,14,20,21-heptazapentacyclo[17.5.2.0^{2,6}.0^{12,16}0.0^{22,26}]hexacosa-1(25),2(6),3,12,15,17,19,22(26), 23-nonaen-14-yl]propanoate (20 mg, 0.041 mmoL, 1 eq) in THE (1 mL) was degassed and purged with N₂ for 3 times, MeLi (1.6 M, 25.5 uL, 1 eq) was added at 0° C., and then the mixture was stirred at 20° C. for 2 h under N₂ atmosphere. On completion, the reaction mixture was quenched by addition sat. NH₄Cl 10 mL at 0° C., and then diluted with H₂O 30 mL and extracted with EA (3*30 mL). The combined organic layers were dried over Na₂SO₄, filtered and concentrated under reduced pressure to give a residue. The crude product was purified by reversed-phase HPLC (Phenomenex C18 150*25 mm*10 um; mobile phase: [water (NH₄HCO₃)-ACN]; B %: 5%-35%, 8 min) to give Ex. 95 (0.58 mg, 0.001 mmol, 3% yield, 92% purity) as a white solid.

### General Method N: Preparation of (12R,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine (Ex. 96) and (12S,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine (Ex. 97)

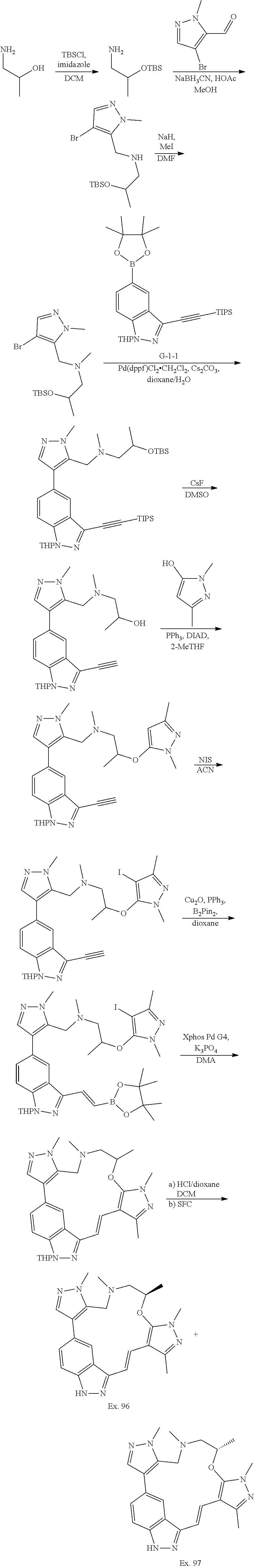

Step 1. To a solution of 1-aminopropan-2-ol (10.0 g, 133 mmol, 10.4 mL, 1 eq) in DCM (100 mL) was added TBSCl (22.1 g, 146 mmol, 18.0 mL, 1.1 eq) and imidazole (18.1 g, 266 mmol, 2 eq) at 0° C. The mixture was stirred at 25° C. for 16 h. On completion, the reaction mixture was diluted with H₂O (100 mL) and extracted with DCM (50 mL*3). The combined organic layers were dried over Na₂SO₄, filtered and the filtrate was concentrated under reduced pressure to give a residue. The residue was purified by column chromatography to give 2-[tert-butyl (dimethyl)silyl]oxypropan-1-amine (13.7 g, 72.4 mmol, 54% yield) as colorless liquid. ¹H NMR (400 MHz, CDCl₃) δ=3.76-3.70 (m, 1H), 2.63-2.50 (m, 2H), 1.07 (d, J=6.0 Hz, 3H), 0.86 (s, 9H), 0.04 (s, 6H).

Step 2. To a solution of 4-bromo-2-methyl-pyrazole-3-carbaldehyde (8.46 g, 44.8 mmol, 1 eq) and 2-[tert-butyl(dimethyl)silyl]oxypropan-1-amine (12.7 g, 67.1 mmol, 1.5 eq) in MeOH (90 mL) was added HOAc (2.69 g, 44.8 mmol, 2.56 mL, 1 eq) to adjust pH to 6 and then NaBH₃CN (5.63 g, 89.5 mmol, 2 eq) was added. The mixture was stirred at 25° C. for 2 h. On completion, the reaction mixture was quenched by water (5 mL) at 25° C., and then filtered and the filtrate was concentrated under reduced pressure to give a residue. The residue was purified by column chromatography to give N-[(4-bromo-2-methyl-pyrazol-3-yl)methyl]-2-[tert-butyl(dimethyl)silyl]oxy-propan-1-amine (19.4 g, 42.8 mmol, 96% yield, 80% purity) as light yellow liquid. LCMS: m/z 364.2 (M+1).

Step 3. To a solution of N-[(4-bromo-2-methyl-pyrazol-3-yl)methyl]-2-[tert-butyl(dimethyl)silyl]oxy-propan-1-amine (18.4 g, 40.6 mmol, 80% purity, 1 eq) in DMF (185 mL) was added NaH (3.24 g, 81.1 mmol, 60% purity, 2 eq) at 0° C. and stirred for 0.5 h, and then MeI (8.63 g, 60.8 mmol, 3.79 mL, 1.5 eq) was added at 0° C. The mixture was stirred at 25° C. for 15.5 h. On completion, the reaction mixture was quenched by sat. NH₄Cl 50 mL at 0° C., and then diluted with H₂O 300 mL and extracted with EA (80 mL*3). The combined organic layers were washed with H₂O (150 mL*2), dried over Na₂SO₄, filtered and the filtrate was concentrated under reduced pressure to give a residue. The residue was purified by column chromatography to give N-[(4-bromo-2-methyl-pyrazol-3-yl)methyl]-2-[tert-butyl(dimethyl)silyl]oxy-N-methyl-propan-1-amine (12.7 g, 33.7 mmol, 83% yield) as colorless liquid. LCMS: m/z 377.8 (M+1).

Step 4. A mixture of N-[(4-bromo-2-methyl-pyrazol-3-yl)methyl]-2-[tert-butyl(dimethyl)silyl]oxy-N-methyl-propan-1-amine (1.00 g, 2.66 mmol, 1 eq), triisopropyl-[2-[1-tetrahydropyran-2-yl-5-(4,4,5,5-tetramethyl-1,3,2-dioxaborolan-2-yl) indazol-3-yl]ethynyl]silane (1.49 g, 2.92 mmol, 1.1 eq), Cs₂CO₃ (2.60 g, 7.97 mmol, 3 eq), Pd(dppf)Cl₂·CH₂Cl₂ (217 mg, 0.266 mmol, 0.1 eq) and H₂O (2 mL) in dioxane (10 mL) was degassed and purged with N₂ for 3 times, and then the mixture was stirred at 80° C. for 2 h under N2 atmosphere. On completion, the reaction mixture was dried over Na₂SO₄, filtered and the filtrate was concentrated under reduced pressure to give a residue. The residue was purified by column chromatography to give 2-[tert-butyl(dimethyl)silyl]oxy-N-methyl-N-[[2-methyl-4-[1-tetrahydropyran-2-yl-3-(2-triisopropylsilylethynyl) indazol-5-yl]pyrazol-3-yl]methyl]propan-1-amine (1.36 g, 2.01 mmol, 76% yield) as yellow oil. LCMS: m/z 678.8 (M+1).

Step 5. To a solution of 2-[tert-butyl(dimethyl)silyl]oxy-N-methyl-N-[[2-methyl-4-|1-tetrahydropyran-2-yl-3-(2-triisopropylsilylethynyl) indazol-5-yl|pyrazol-3-yl| methyl]propan-1-amine (6.52 g, 9.61 mmol, 1 eq) in DMSO (70 ml) was added CsF (17.5 g, 115 mmol, 12 eq). The mixture was stirred at 40° C. for 16 h. On completion, the reaction mixture was diluted with H₂O 150 mL and extracted with EA (50 mL*3). The combined organic layers were washed with H₂O (150 mL*2), dried over Na₂SO₄, filtered and the filtrate was concentrated under reduced pressure to give a residue. The residue was purified by column chromatography to give 1-[[4-(3-ethynyl-1-tetrahydropyran-2-yl-indazol-5-yl)-2-methyl-pyrazol-3-yl]methyl-methyl-amino]propan-2-ol (1.99 g, 4.89 mmol, 51% yield) as orange oil. LCMS: m/z 407.9 (M+1).

Step 6. A mixture of 1-[[4-(3-ethynyl-1-tetrahydropyran-2-yl-indazol-5-yl)-2-methyl-pyrazol-3-yl]methyl-methyl-amino]propan-2-ol (800 mg, 1.96 mmol, 1 eq), 2,5-dimethylpyrazol-3-ol (484 mg, 4.32 mmol, 2.2 eq), PPh₃ (1.13 g, 4.32 mmol, 2.2 eq) in 2-MeTHF (4 mL) was stirred at 25° C. for 0.5 h, and then DIAD (873 mg, 4.32 mmol, 840 μL, 2.2 eq) was added at 0° C., the mixture was stirred at 25° C. for 2 h under N2 atmosphere. On completion, the reaction mixture was concentrated under reduced pressure to give a residue. The residue was purified by column chromatography to give 2-(2,5-dimethylpyrazol-3-yl)oxy-N—[[4-(3-ethynyl-1-tetrahydropyran-2-yl-indazol-5-yl)-2-methyl-pyrazol-3-yl]methyl]-N-methyl-propan-1-amine (410 mg, 0.817 mmol, 42% yield) as yellow oil. LCMS: m/z 502.1 (M+1).

Step 7. To a solution of 2-(2,5-dimethylpyrazol-3-yl)oxy-N-[[4-(3-ethynyl-1-tetrahydropyran-2-yl-indazol-5-yl)-2-methyl-pyrazol-3-yl]methyl]-N-methyl-propan-1-amine (360 mg, 0.718 mmol, 1 eq) in ACN (5 mL) was added NIS (153 mg, 0.682 mmol, 0.95 eq) at 0° C. The mixture was stirred at 0° C. for 2 h. On completion, the reaction mixture was quenched by sat. Na₂SO₃ 8 mL at 0° C., and then diluted with H₂O 30 mL and extracted with EA 30 mL (10 mL*3). The combined organic layers were dried over Na₂SO₄, filtered and concentrated under reduced pressure to give a residue. The residue was purified by column chromatography to give N-[[4-(3-ethynyl-1-tetrahydropyran-2-yl-indazol-5-yl)-2-methyl-pyrazol-3-yl]methyl]-2-(4-iodo-2,5-dimethyl-pyrazol-3-yl)oxy-N-methyl-propan-1-amine (276 mg, 0.440 mmol, 61% yield) as yellow solid. LCMS: m/z 628.1 (M+1).

Step 8. A mixture of N-[[4-(3-ethynyl-1-tetrahydropyran-2-yl-indazol-5-yl)-2-methyl-pyrazol-3-yl]methyl]-2-(4-iodo-2,5-dimethyl-pyrazol-3-yl)oxy-N-methyl-propan-1-amine (266 mg, 0.424 mmol, 1 eq), 4,4,5,5-tetramethyl-2-(4,4,5,5-tetramethyl-1,3,2-dioxaborolan-2-yl)-1,3,2-dioxaborolane (215 mg, 0.848 mmol, 2 eq), PPh₃ (111 mg, 0.424 mmol, 1 eq), Cu₂O (30.3 mg, 0.212 mmol, 21.7 uL, 0.5 eq) in dioxane (3 mL) was degassed and purged with N₂ for 3 times, and then the mixture was stirred at 80° C. for 16 h under N₂ atmosphere. On completion, the reaction mixture was filtered and the solution was diluted with H₂O 20 mL and extracted with EA 30 mL (10 mL*3). The combined organic layers were dried over Na₂SO₄, filtered and the filtrate was concentrated under reduced pressure to give a residue. The residue was purified by column chromatography to give 2-(4-iodo-2,5-dimethyl-pyrazol-3-yl)oxy-N-methyl-N-[[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]methyl]propan-1-amine (264 mg, 0.349 mmol, 82% yield) as colorless oil. LCMS: m/z 756.1 (M+1).

Step 9. A mixture of 2-(4-iodo-2,5-dimethyl-pyrazol-3-yl)oxy-N-methyl-N-[[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]methyl]propan-1-amine (201 mg, 0.266 mmol, 1 eq), Xphos Pd G4 (22.9 mg, 0.027 mmol, 0.1 eq), K₃PO₄ (169 mg, 0.798 mmol, 3 eq) in DMA (4 mL) was degassed and purged with N₂ for 3 times, and then the mixture was stirred at 80° C. for 2 h under N₂ atmosphere. On completion, the reaction mixture was diluted with H₂O 50 mL and extracted with EA 60 mL (20 mL*3). The combined organic layers were dried over Na₂SO₄, filtered and concentrated under reduced pressure to give a residue. The residue was purified by column chromatography to give (17E)-5,8,10,13,15-pentamethyl-21-tetrahydropyran-2-yl-11-oxa-4,5,8,13,14,20,21-heptazapentacyclo [17.5.2.02,6.012,16.022,26]hexacosa-1(25),2(6),3,12(16),14,17,19,22(26),23-nonaene (32.0 mg, 0.064 mmol, 24% yield) as colorless oil. ¹H NMR (400 MHz, MeOD-d₄) δ=9.07-8.31 (m, 1H), 7.76-7.61 (m, 1H), 7.57-7.41 (m, 2H), 5.66 (s, 1H), 4.67-4.56 (m, 1H), 3.99 (s, 1H), 3.92-3.79 (m, 3H), 3.76-3.67 (m, 3H), 3.61-3.52 (m, 3H), 3.45-3.39 (m, 1H), 3.31 (s, 1H), 2.98-2.85 (m, 1H), 2.81 (d, J=0.8 Hz, 1H), 2.37-2.30 (m, 3H), 2.21 (s, 3H), 2.10-1.97 (m, 3H), 1.88-1.82 (m, 2H), 1.23-1.11 (m, 3H), 1.02-0.79 (m, 1H). LCMS: m/z 502.0 (M+1).

Step 10. To a solution of (17E)-5,8,10,13,15-pentamethyl-21-tetrahydropyran-2-yl-11-oxa-4,5,8,13,14,20,21-heptazapentacyclo[17.5.2.02,6.012,16.022,26]hexacosa-1(25),2(6), 3,12(16),14,17,19,22(26),23-nonaene (30.0 mg, 0.060 mmol, 1 eq) in DCM (2 mL) was added HCl/dioxane (4 M, 2 mL, 134 eq). The mixture was stirred at 25° C. for 1 h.

On completion, the reaction mixture was concentrated under reduced pressure to remove solvent. The residue was purified by prep-HPLC (column: DAICEL CHIRALPAK AD (250 mm*30 mm, 10 um); mobile phase: [0.1% NH₃·H₂O MeOH]; B %: 40%-40%, C2.55; 30 min) to give crude material (17.4 mg, 0.042 mmol, 70% yield) as a white solid. This material was further purified by SFC (Column: Chiralcel OD-3 50×4.6 mm I.D., 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA; Column Temp: 35 C; Back Pressure: 100 Bar”) to give two peak. Each peak was purified by Prep-HPLC (column: Phenomenex luna C18 150*25 mm*10 um; mobile phase: [water (FA)-ACN]; B %: 20%-50%, 10 min) to give arbitrarily assigned Ex. 96 and Ex. 97.

### Preparation of (rac)-(3S,4S)-1-methyl-4-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]pyrrolidin-3-ol (Ex. 98)

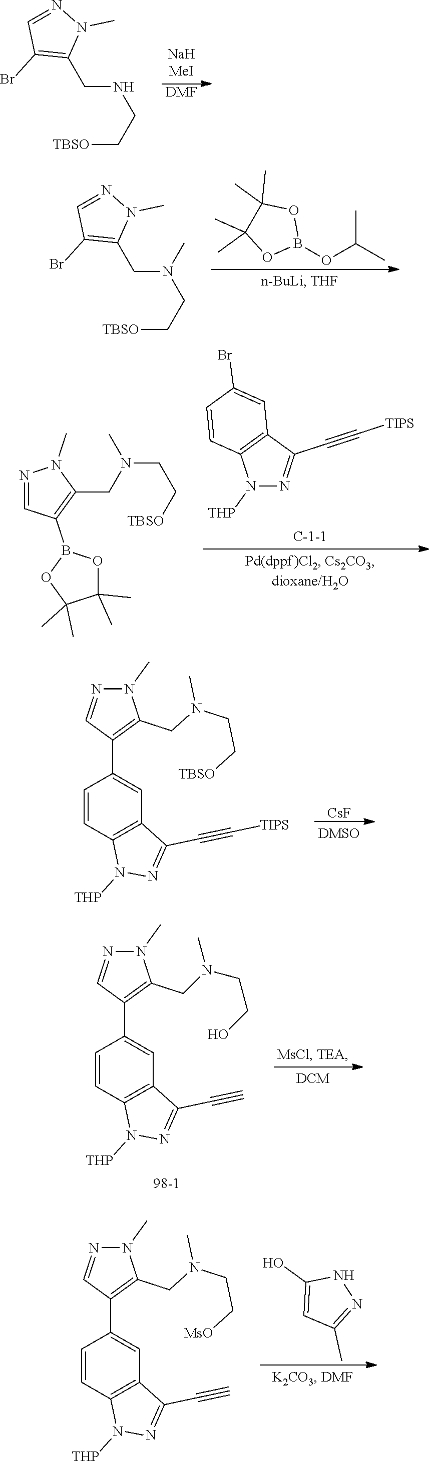

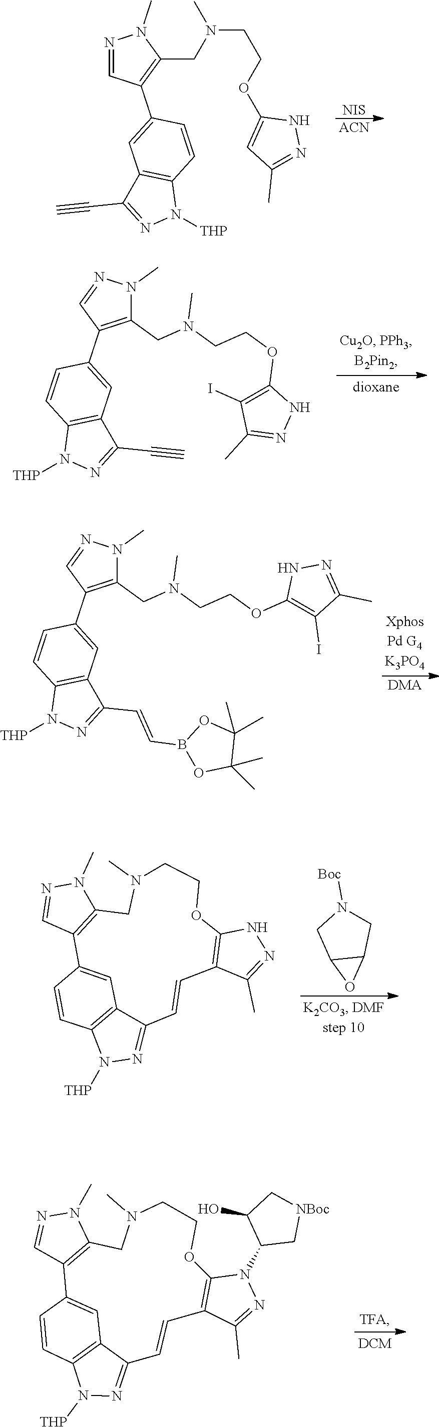

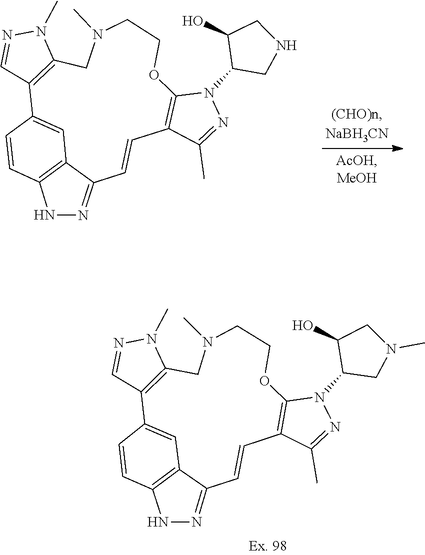 [SMILES: Cc1nn([C@H]2CNC[C@@H]2O)c2c1/C=C/c1n[nH]c3ccc(cc13)-c1cnn(C)c1CN(C)CCO2 ; Cc1nn([C@H]2CN(C)C[C@@H]2O)c2c1/C=C/c1n[nH]c3ccc(cc13)-c1cnn(C)c1CN(C)CCO2]

Steps 1 was conducted in a similar manner to step 3 in General Method N.

Step 2 was conducted in a similar manner to step 3 in the synthesis of intermediate B-2-4.

Steps 3 and 4 were conducted in a similar manner to steps 4 and 5 in General Method N.

Step 5-8 were conducted in a similar manner to steps 3-6 in General Method G.

Step 9 were conducted in a similar manner to step 9 in General Method N.

Step 10. To a solution of (17E)-5,8,15-trimethyl-21-tetrahydropyran-2-yl-11-oxa-4,5,8,13,14,20,21-heptazapentacyclo[17.5.2.0^{2,6}.0^{12,16}0.0^{22,26}]hexacosa-1(25),2(6),3,12 (16),14,17,19,22(26),23-nonaene (80.0 mg, 0.168 mmol, 1 eq) and tert-butyl 6-oxa-3-azabicyclo[3.1.0]hexane-3-carboxylate (187 mg, 1.01 mmol, 6 eq) in DMF (6 mL) was added Cs₂CO₃ (165 mg, 0.506 mmol, 3 eq). The mixture was stirred at 70° C. for 12 h. On completion, the mixture was quenched with water (10 mL) and extracted with EA (15 mL×3), the organic layer was concentrated in vacuum to give the residue. The residue was purified by column chromatography (SiO₂, Petroleum ether/Ethyl acetate=2:1 to 2:1) to give (rac)-tert-butyl (4R)-3-hydroxy-4-[(17E)-5,8,15-trimethyl-21-tetrahydropyran-2-yl-11-oxa-4,5,8,13,14,20,21-heptazapentacyclo[17.5.2.0^{2,6}.0^{12,16}0.0^{22,26}]hexacosa-1(25),2(6),3,12(16),14,17,19,22(26),23-nonaen-13-yl]pyrrolidine-1-carboxylate (50.0 mg, 0.076 mmol, 44% yield) as a yellow oil. LCMS: m/z 659.6 (M+1).

Step 11 was conducted in a similar manner to the last step in General Method C.

Step 12. To a solution of 4-[(17E)-5,8,15-trimethyl-11-oxa-4,5,8,13,14,20,21-heptazapentacyclo[17.5.2.0^{2,6}.0^{12,16}0.0^{22,26}]hexacosa-1(25),2(6),3,12,15,17,19,22(26), 23-nonaen-14-yl]pyrrolidin-3-ol (40 mg, 0.084 mmol, 1 eq) in MeOH (1 mL) was added (CHO)ₙ (8.61 mg, 0.253 mmol, 3 eq) and AcONa (69.1 mg, 10 eq) and then NaBH₃CN (10.5 mg, 0.168 mmol, 2 eq) was added at 25° C. The mixture was stirred at 25° C. for 4 h. On completion, the mixture was quenched with water (10 mL) and extracted with EA (15 mL×3), the organic layer was concentrated in vacuum to give the residue. The residue was further purification by pre-HPLC (column: Welch Xtimate C18 150*25 mm*5 um; mobile phase: [water(TFA)-ACN]; B %: 0%-27%, 10 min) to give (rac)-(3S,4S)-1-methyl-4-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]pyrrolidin-3-ol (3.82 mg, 0.008 mmol, 9% yield) as a white solid.

### Preparation of 2-methyl-4-[1-tetrahydropyran-2-yl-3-(2-triisopropylsilylethynyl) indazol-5-yl]pyrazole-3-carbaldehyde (I-99)

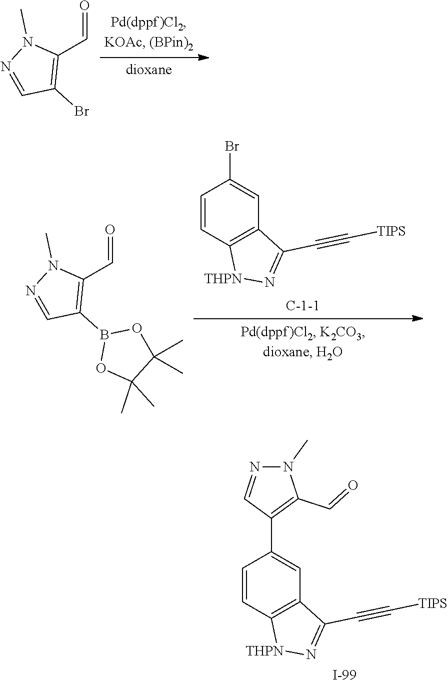

Step 1. A mixture of 4-bromo-2-methyl-pyrazole-3-carbaldehyde (20 g, 106 mmol, 1 eq), 4,4,5,5-tetramethyl-2-(4,4,5,5-tetramethyl-1,3,2-dioxaborolan-2-yl)-1,3,2-dioxaborolane (53.7 g, 212 mmol, 2 eq), Pd(dppf)Cl₂ (7.74 g, 10.6 mmol, 0.1 eq), KOAc (31.2 g, 317 mmol, 3 eq) in dioxane (200 mL) was degassed and purged with N2 for 3 times, and then the mixture was stirred at 100° C. for 2 h under N2 atmosphere. On completion, the reaction mixture was partitioned between ethyl acetate (200 mL×3) and water (500 mL), the combined organic phase was dried over anhydrous sodium sulfate, filtered and concentrated to give a residue. The residue was purified by column chromatography to give 2-methyl-4-(4, 4, 5, 5-tetramethyl-1, 3, 2-dioxaborolan-2-yl) pyrazole-3-carbaldehyde (32 g, 94.9 mmol, 90% yield) as light-yellow solid. ¹H NMR (400 MHz, DMSO-d6) δ=10.11 (s, 1H), 7.73 (s, 1H), 4.08 (s, 3H), 1.30 (s, 12H); LCMS: m/z 491.3 (M+1).

Step 2. To a solution of 2-methyl-4-(4,4,5,5-tetramethyl-1,3,2-dioxaborolan-2-yl)pyrazole-3-carbaldehyde (9.97 g, 42.3 mmol, 1.3 eq) and 2-(5-bromo-1-tetrahydropyran-2-yl-indazol-3-yl)ethynyl-triisopropyl-silane (15 g, 32.5 mmol, 1 eq) in dioxane (150 mL) and H₂O (50 mL) was added Pd(dppf)Cl₂ (2.38 g, 3.25 mmol, 0.1 eq) and K₂CO₃ (13.5 g, 97.5 mmol, 3 eq). The mixture was stirred at 90° C. for 2 h. On completion, the reaction mixture was diluted with H₂O (500 mL) and extracted with EtOAc (200 mL×3). The combined organic layers were washed with brine 200 mL, dried over Na₂SO₄, filtered and concentrated under reduced pressure to give a residue. The residue was purified by column chromatography to give 2-methyl-4-[1-tetrahydropyran-2-yl-3-(2-triisopropylsilylethynyl) indazol-5-yl]pyrazole-3-carbaldehyde (5.5 g, 9.30 mmol, 29% yield) as a white solid. LCMS: m/z 491.3 (M+1).

### Preparation of (11S,17E)-8,11,14,16-tetramethyl-2,9,10,11,12,14-hexahydro-8H-3,5-ethenotripyrazolo[3,4-f:4′,3′-j:4″,3″-n][1,4]oxazacyclopentadecine (Ex. 99)

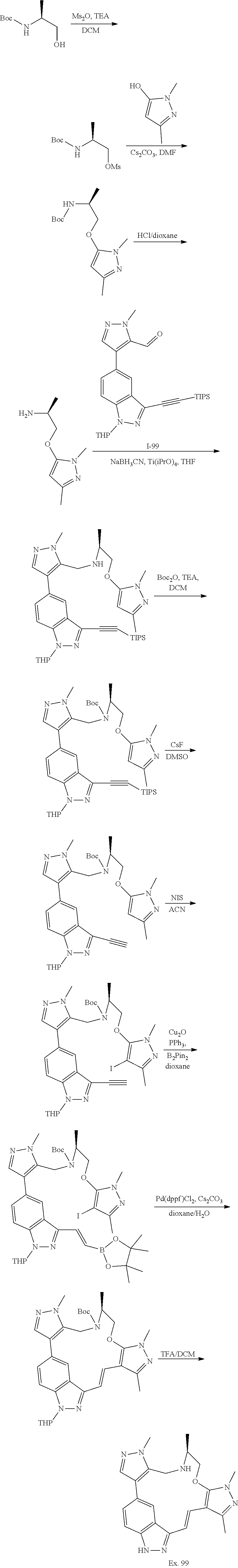

Step 1. To a solution of tert-butyl N-[(1S)-2-hydroxy-1-methyl-ethyl]carbamate (6 g, 34.2 mmol, 1 eq) in DCM (50 mL) was added TEA (6.93 g, 68.5 mmol, 9.5 mL, 2 eq), and then methylsulfonyl methanesulfonate (11.9 g, 68.5 mmol, 2 eq) was added at 0° C. The mixture was stirred at 25° C. for 1 h. On completion, the reaction mixture was partitioned between dichloromethane (100 ml) and water (300 mL), the combined organic phase was dried over anhydrous sodium sulfate, filtered and concentrated to give a residue to give [(2S)-2-(tert-butoxycarbonylamino)propyl]methanesulfonate (8 g, crude) as yellow solid. ¹H NMR (400 MHz, DMSO-d₆) δ=5.75 (s, 1H), 4.03 (d, J=5.6 Hz, 2H), 3.74 (td, J=6.4, 13.2 Hz, 1H), 3.16 (s, 3H), 1.38 (s, 9H), 1.06 (d, J=6.8 Hz, 3H).

Step 2. To a solution of [(2S)-2-(tert-butoxycarbonylamino) propyl]methanesulfonate (8 g, 31.6 mmol, i eq) and 2, 5-dimethylpyrazol-3-ol (4.25 g, 37.9 mmol, 1.2 eq) in DMF (80 mL) was added Cs₂CO₃ (20.6 g, 63.2 mmol, 2 eq). The mixture was stirred at 25° C. for 16 h. On completion, the reaction mixture was diluted with H₂O (250 mL) and extracted with DCM (100 mL×3). The combined organic layers were washed with brine 120 mL, dried over Na₂SO₄, filtered and concentrated under reduced pressure to give a residue. The residue was purified by column chromatography to give tert-butyl N-[(1S)-2-(2, 5-dimethylpyrazol-3-yl) oxy-1-methyl-ethyl]carbamate (4 g, 13.5 mmol, 91% purity) as a yellow oil. ¹H NMR (400 MHz, DMSO-d₆) δ=5.41 (s, 1H), 3.84 (s, 2H), 3.83-3.77 (m, 1H), 3.43 (s, 3H), 2.02 (s, 3H), 1.38 (s, 9H), 1.08 (d, J=6.4 Hz, 3H). LCMS: m/z 270.2(M+1)

Step 3. To a solution of tert-butyl N-[(1 S)-2-(2, 5-dimethylpyrazol-3-yl) oxy-1-methyl-ethyl]carbamate (4 g, 14.9 mmol, 1 eq) in HCl/dioxane (40 mL) and DCM (40 mL). The mixture was stirred at 25° C. for 2 h. On completion, the mixture was concentrated to give a residue to give (2S)-1-(2, 5-dimethylpyrazol-3-yl) oxypropan-2-amine (3 g, crude) as a white solid. LCMS: m/z 170.3(M+1)

Step 4. To a solution of 2-methyl-4-[1-tetrahydropyran-2-yl-3-(2-triisopropylsilylethynyl) indazol-5-yl]pyrazole-3-carbaldehyde (1.5 g, 3.06 mmol, 1 eq) and (2S)-1-(2,5-dimethylpyrazol-3-yl)oxypropan-2-amine (1.26 g, 6.11 mmol, 2 eq, HCl) in THF (20 mL) was added Ti(i-PrO)₄ (1.74 g, 6.11 mmol, 1.8 mL, 2 eq) at 25° C. The mixture was stirred at 25° C. for 1 hour, and then NaBH₃CN (288 mg, 4.59 mmol, 1.5 eq) was added. The mixture was stirred at 25° C. for 1 h. On completion, the mixture was quenched with water (30 mL) and extracted with EA (200 ml), filtered and filter liquor was concentrated to give a residue. The residue was purified by column chromatography to give (2S)-1-(2,5-dimethylpyrazol-3-yl)oxy-N—[[2-methyl-4-[1-tetrahydropyran-2-yl-3-(2-triisopropylsilylethynyl) indazol-5-yl]pyrazol-3-yl]methyl]propan-2-amine (670 mg, 0.520 mmol, 17% yield) as yellow oil. LCMS: m/z 644.7 (M+1)

Step 5. To a solution of (2S)-1-(2, 5-dimethylpyrazol-3-yl) oxy-N-[[2-methyl-4-[1-tetrahydropyran-2-yl-3-(2- triisopropylsilylethynyl) indazol-5-yl]pyrazol-3-yl]methyl]propan-2-amine (600 mg, 0.932 mmol, 1 eq) and Boc₂O (407 mg, 1.86 mmol, 2 eq) in DCM (5 mL) was added TEA (283 mg, 2.80 mmol, 3 eq). The mixture was stirred at 25° C. for 16 h. On completion, the reaction mixture was diluted with H₂O (50 mL) and extracted with EtOAc (20 mL×3). The combined organic layers were washed with brine 20 mL, dried over Na₂SO₄, filtered and concentrated under reduced pressure to give a residue. The residue was purified by column chromatography to give tert-butyl N-[(1S)-2-(2,5-dimethylpyrazol-3-yl)oxy-1-methyl-ethyl]-N-[[2-methyl-4-[1-tetrahydropyran-2-yl-3-(2-triisopropylsilylethynyl) indazol-5-yl]pyrazol-3-yl]methyl]carbamate (400 mg, 0.425 mmol, 46% yield) as a white oil. LCMS: m/z 744.5 (M+1)

Step 6. To a solution of tert-butyl N-[(1S)-2-(2, 5-dimethylpyrazol-3-yl) oxy-1-methyl-ethyl]-N-[[2-methyl-4-[1-tetrahydropyran-2-yl-3-(2-triisopropylsilylethynyl) indazol-5-yl]pyrazol-3-yl]methyl]carbamate (400 mg, 0.538 mmol, 1 eq) in DMSO (5 mL) was added CsF (245 mg, 1.61 mmol, 3 eq). The mixture was stirred at 25° C. for 16 h. On completion, the reaction mixture was diluted with H₂O (50 mL) and extracted with EtOAc (20 mL×3). The combined organic layers were washed with brine 50 mL, dried over Na₂SO₄, filtered and concentrated under reduced pressure to give tert-butyl N-[(1S)-2-(2,5-dimethylpyrazol-3-yl)oxy-1-methyl-ethyl]-N-[[4-(3-ethynyl-1-tetrahydropyran-2-yl-indazol-5-yl)-2-methyl-pyrazol-3-yl]methyl]carbamate (360 mg, 0.515 mmol, 96% yield) was obtained as a yellow oil. LCMS: m/z 588.5 (M+1)

Step 7. To a solution of tert-butyl N-[(1S)-2-(2, 5-dimethylpyrazol-3-yl) oxy-1-methyl-ethyl]-N-[[4-(3-ethynyl-1-tetrahydropyran-2-yl-indazol-5-yl)-2-methyl-pyrazol-3-yl]methyl]carbamate (360 mg, 0.613 mmol, 1 eq) in ACN (2 mL) was added NIS (152 mg, 0.674 mmol, 1.1 eq). The mixture was stirred at 25° C. for 4 h. On completion, the reaction mixture was diluted with H₂O (50 mL) and extracted with EtOAc (20 mL×3). The combined organic layers were washed with brine 50 mL, dried over Na₂SO₄, filtered and concentrated under reduced pressure to give a residue. The residue was purified by column chromatography to give tert-butyl N-[[4-(3-ethynyl-1-tetrahydropyran-2-yl-indazol-5-yl)-2-methyl-pyrazol-3-yl]methyl]-N-[(1S)-2-(4-iodo-2,5-dimethyl-pyrazol-3-yl)oxy-1-methyl-ethyl]carbamate (380 mg, 0.447 mmol, 73% yield) as a yellow oil. LCMS: m/z 714.1 (M+1)

The remaining steps were conducted in a manner similar to those described in General Method H to give Ex. 99

### Preparation of ethyl 2-[(17E)-5,7,15-trimethyl-21-tetrahydropyran-2-yl-8,11-dioxa-4,5,13,14,20,21-hexazapentacyclo[17.5.2.0^{2,6}0.0^{12,16}0.0^{22,26}]hexacosa-1(25),2(6),3,12(16),14,17,19,22(26),23-nonaen-13-yl]acetate (1-100)

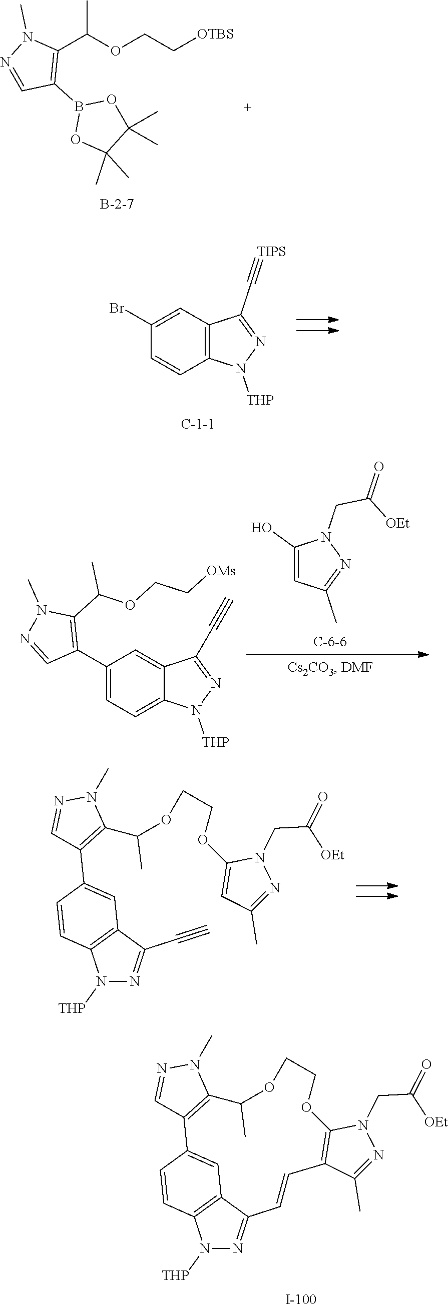

I-100 was prepared in a similar manner to those described in General Method C.

### Preparation of (17E)-8,9,16-trimethyl-14-[2-(pyrrolidin-1-yl)ethyl]-2,8,9,11,12,14-hexahydro-3,5-etheno[1,4]dioxacyclopentadecino[11,10-c:15,14-c′:6,7-c″]tripyrazole (Ex. 100)

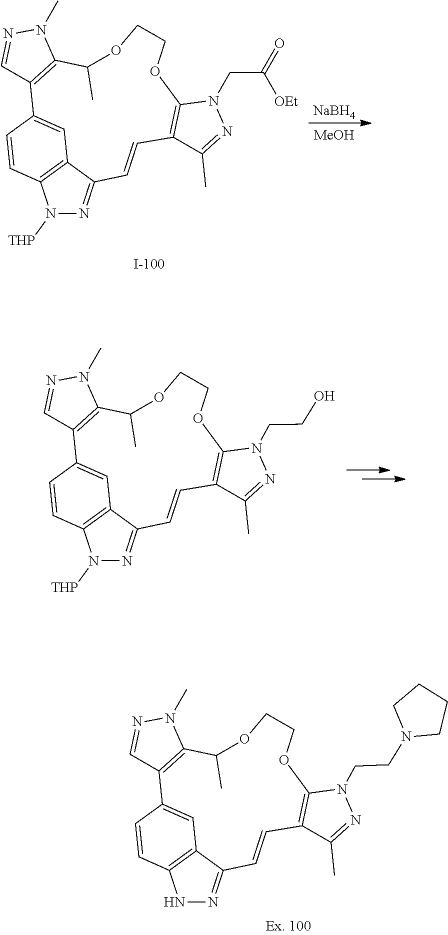

I-100 was converted to Ex. 100 following procedures described in General Method D and F using the appropriate amine.

### Preparation of [(10S,17E)-8,10,14-trimethyl-2,11,12,14-tetrahydro-8H,10H-3,5-etheno[1,5]dioxacyclopentadecino[10,11-c:15,14-c′:6,7-c′]tripyrazol-16-yl]methanol (Ex. 101)

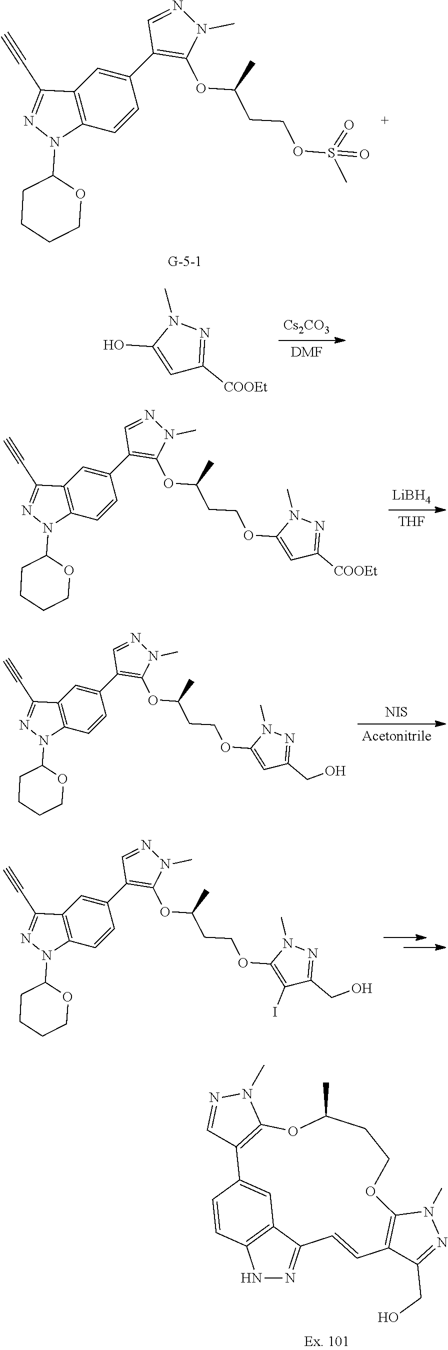

Step 1. To ethyl 5-hydroxy-1-methyl-pyrazole-3-carboxylate (180.05 mg, 1.06 mmol) in DMF (5 mL) was added base, Cesium carbonate (517.11 mg, 1.59 mmol) and stirred for 30 minutes followed by [(3S)-3-[4-(3-ethynyl-1-tetrahydropyran-2-yl-indazol-5-yl)-2-methyl-pyrazol-3-yl]oxybutyl]methanesulfonate (250 mg, 0.529 mmol). Stirred at 22° C. for 22 hr. Diluted with DCM and cooled. Solids were filtered and washed with DCM. Reaction was diluted with DCM and water (25 mL) and the layers were separated. The aqueous layer was extracted again with DCM (2×10 mL). The combined organic layer was washed with brine and dried over sodium sulfate. Flash column chromatography (automated system, 12 g, 50-100% EA in Hexanes) provided ethyl 5-[(3S)-3-[4-(3-ethynyl-1-tetrahydropyran-2-yl-indazol-5-yl)-2-methyl-pyrazol-3-yl]oxybutoxy]-1-methyl-pyrazole-3-carboxylate (300.63 mg, 0.55 mmol, quantitative yield). Take forward without further purification. LCMS: m/z 547.0 (M+1)

Step 2. To ethyl 5-[(3S)-3-[4-(3-ethynyl-1-tetrahydropyran-2-yl-indazol-5-yl)-2-methyl-pyrazol-3-yl]oxybutoxy]-1-methyl-pyrazole-3-carboxylate (250 mg, 0.457 mmol) in THE (3 mL) was added lithium borohydride (13.56 mg, 0.686. mmol, 12.63 μL). Stir at 0° C. for 18 hr as temp increases to RT. reaction was quenched with 1 mL of water and 1 mL of 2 M NaOH (aq), stir vigorously and worked up with DCM and water (10 mL each). The aqueous layer was extracted again with DCM (2×10 mL). The combined organic layer was washed with brine and dried over sodium sulfate. Flash column chromatography (automated system, 12 g silica, 0-60% 1:3 DCM:MeOH in EA) provided [5-[(3S)-3-[4-(3-ethynyl-1-tetrahydropyran-2-yl-indazol-5-yl)-2-methyl-pyrazol-3-yl]oxybutoxy]-1-methyl-pyrazol-3-yl]methanol (75 mg, 0.147 mmol, 33% yield). LCMS: m/z 505.2 (M+1)

Step 3. To [5-[(3S)-3-[4-(3-ethynyl-1-tetrahydropyran-2-yl-indazol-5-yl)-2-methyl-pyrazol-3-yl]oxybutoxy]-1-methyl-pyrazol-3-yl]methanol (75 mg, 0.149 mmol) in Acetonitrile (3 mL) was added NIS (36.79 mg, 0.164 mmol). Stir at 22° C. for 18 hr. Quenched with water and worked up with DCM and water (10 mL) and the layers were separated. The aqueous layer was extracted again with DCM (2×5 mL). The combined organic layer was washed with brine and dried over sodium sulfate. Flash column chromatography twice (automated system, 12 g silica, 0-10% Methanol in DCM) provided [5-[(3S)-3-[4-(3-ethynyl-1-tetrahydropyran-2-yl-indazol-5-yl)-2-methyl-pyrazol-3-yl]oxybutoxy]-4-iodo-1-methyl-pyrazol-3-yl]methanol (72.22 mg, 0.115 mmol, 77% yield). LCMS: m/z 630.6 (M+1)

The remaining steps were conducted according to General Method L to give Ex. 101.

### Preparation of N¹—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c′]tripyrazol-15-yl]cyclobutyl}-N¹,N—,N²-trimethylethane-1,2-diamine (Ex. 102)

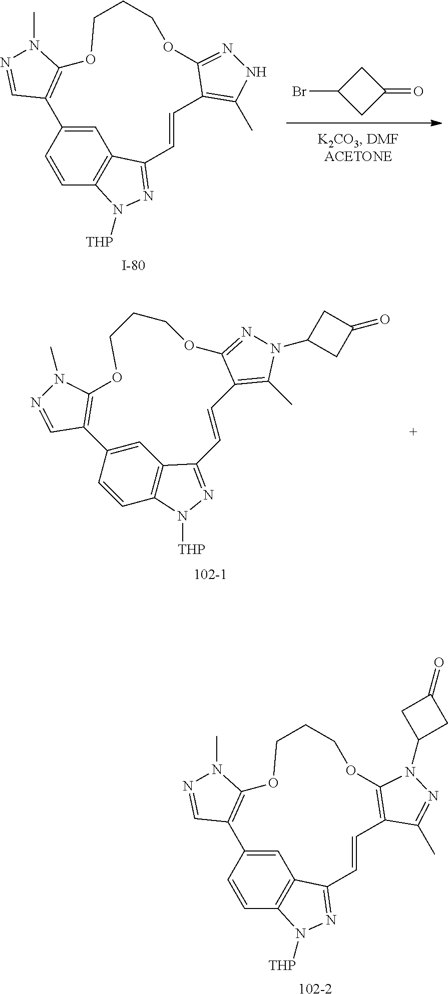

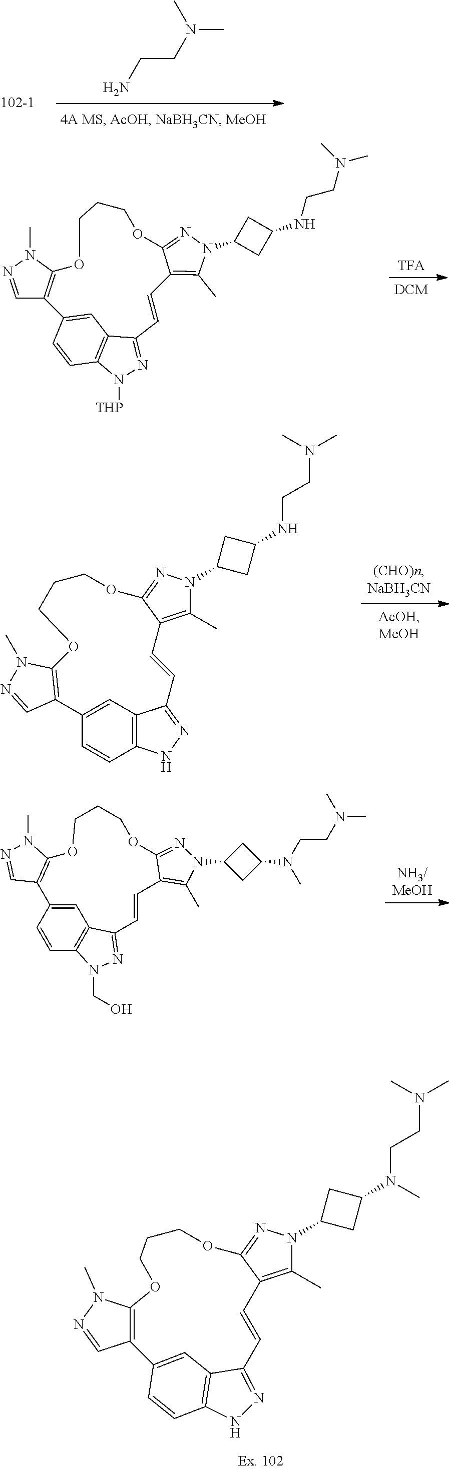

Step 1. To a mixture of (17E)-5,15-dimethyl-21-tetrahydropyran-2-yl-7,11-dioxa-4,5,13,14,20,21-hexazapentacyclo[17.5.2.0^{2,6}.0^{12,16}0.0^{22,26}]hexacosa-1(25),2(6),3,12, 15,17,19,22(26),23-nonaene (65.0 mg, 0.141 mmol, 1 eq,) and 3-bromocyclobutanone (18.9 mg, 0.127 mmol, 0.9 eq) in DMF (1 mL) and acetone (3 mL) was added K₂CO₃ (19.5 mg, 0.141 mmol, 1 eq), the mixture was stirred at 23° C. for 10 h. On completion, the reaction mixture was partitioned between 2-MeTHF (3 mL×3) and water (3 mL), the combined organic phase was dried over anhydrous sodium sulfate, filtered and concentrated to give a residue. The residue was purified by column chromatography (SiO₂, PE/THF=1:0 to 1:2) to give 3-[(17E)-5,15-dimethyl-21-tetrahydropyran-2-yl-7,11-dioxa-4,5,13,14,20,21-hexazapentacyclo [17.5.2.0^{2,6}.0^{12,16}0.0^{22,26}]hexacosa-1(25),2(6),3,12,15,17,19,22(26),23-nonaen-14-yl]cyclobutanone (32.0 mg, 0.061 mmol, 42% yield) as white solid. LCMS: m/z 529.1 (M+1) and its regioisomer, 102-2, as a white solid. LCMS: m/z 529.1 (M+1)

To a mixture of 3-[(17E)-5,15-dimethyl-21-tetrahydropyran-2-yl-7,11-dioxa-4,5,13,14,20,21-hexazapentacyclo[17.5.2.0^{2,6}.0^{12,16}0.0^{22,26}]hexacosa-1(25),2(6),3,12, 15,17,19,22(26),23-nonaen-14-yl]cyclobutanone (20.0 mg, 0.038 mmol, 1 eq), N′,N′-dimethylethane-1,2-diamine (4.00 mg, 0.045 mmol, 1.2 eq) and 4A MS (0.038 mmol, 1 eq) in MeOH (0.5 mL) was added AcOH to adjust pH=6, the reaction mixture was stirred at 23° C. for 0.5 h, then NaBH₃CN (4.76 mg, 0.076 mmol, 2 eq) was added at 23° C., the reaction mixture was stirred at 23° C. for 2 h. On completion, the mixture was filtered and concentrated to give a residue. The crude product was purified by prep-HPLC purification (column: Welch Ultimate C18 150*25 mm*5 um; mobile phase: [water(TFA)-ACN]; B %: 18%-48%,10 min) to give N-[3-[(17E)-5,15-dimethyl-21-tetrahydropyran-2-yl-7,11-dioxa-4,5,13,14,20,21-hexazapentacyclo[17.5.2.0^{2,6}.0^{12,16}. 0^{22,26}]hexacosa-1(25),2(6),3,12,15,17,19,22(26),23-nonaen-14-yl]cyclobutyl]-N′,N′-dimethyl-ethane-1,2-diamine (11.0 mg, 0.018 mmol, 48% yield) as green gum. LCMS: m/z 601.2 (M+1)

Step 3. To a solution of N-[3-[(17E)-5,15-dimethyl-21-tetrahydropyran-2-yl-7,11-dioxa-4,5,13,14,20,21-hexazapentacyclo[17.5.2.0^{2,6}.0^{12,16}0.0^{22,26}]hexacosa-1(25),2(6),3,12, 15,17,19,22(26),23-nonaen-14-yl]cyclobutyl]-N′,N′-dimethyl-ethane-1,2-diamine (11.0 mg, 0.0183 mmol, 1 eq) and DCM (0.5 ml) was added in TFA (41.8 mg, 0.366 mmol, 20 eq), the resulting mixture was stirred at 23° C. for 1.5 h. On completion, the mixture was concentrated to give a residue. The crude product was purified by prep-HPLC purification (column: Welch Ultimate C18 150*25 mm*5 um; mobile phase: |water(TFA)-ACN]; B %: 8%-38%,10 min) to give N-[3-[(17E)-5,15-dimethyl-7,11-dioxa-4,5,13,14,20,21-hexazapentacyclo[17.5.2.0^{2,6}0.0^{12,16}0.0^{22,26}]hexacosa-1(25),2(6),3, 12,15,17,19,22(26),23-nonaen-14-yl]cyclobutyl]-N′,N′-dimethyl-ethane-1,2-diamine (8.00 mg, 0.015 mmol, 84% yield) as green solid. LCMS: m/z 517.0 (M+1)

Step 4. The mixture of N-[3-[(17E)-5,15-dimethyl-7,11-dioxa-4,5,13,14,20,21-hexazapentacyclo[17.5.2.0^{2,6}.0^{12,16}0.0^{22,26}]hexacosa-1(25),2(6),3,12,15,17,19,22(26), 23-nonaen-14-yl]cyclobutyl]-N′,N′-dimethyl-ethane-1,2-diamine (8.00 mg, 0.015 mmol, 1 eq) and formaldehyde (4.65 mg, 0.154 mmol, 10 eq) in MeOH (0.5 mL) was added AcOH to adjust pH=6, then the mixture was stirred at 25° C. for 0.5 h, Then NaBH₃CN (1.95 mg, 0.031 mmol, 2 eq) was added to the mixture and stirred for another 12 h at 25° C. On completion, the mixture was quenched with water (1 mL) and concentrated in vacuo to give [(17E)-14-[3-[2-(dimethylamino)ethyl-methyl-amino]cyclobutyl]-5,15-dimethyl-7,11-dioxa-4,5,13,14,20,21-hexazapentacyclo[17.5.2.0^{2,6}.0^{12,16}0.0^{22,26}]hexacosa-1(25),2(6),3,12,15,17,19,22(26),23-nonaen-21-yl]methanol (5.00 mg, 0.009 mmol, 57% yield) as a green solid. LCMS: m/z 561.2 (M+1)

Step 5. The solution of [(17E)-14-[3-[2-(dimethylamino)ethyl-methyl-amino]cyclobutyl]-5,15-dimethyl-7,11-dioxa-4,5,13,14,20,21-hexazapentacyclo[17.5.2.0^{2,6}.0^{12,16}0.0^{22,26}]hexacosa-1(25),2(6),3,12,15,17,19,22(26),23-nonaen-21-yl]methanol (4.00 mg, 0.007 mmol, 1 eq) in NH₃/MeOH (0.5 mL) was stirred at 25° C. for 1 h. On completion, the mixture was concentrated to give a residue. The crude product was purified by prep-HPLC purification (column: Welch Xtimate C18 150*25 mm*5 um; mobile phase: [water(TFA)-ACN]; B %: 3%-33%,10 min) to give Ex. 102 (2.33 mg, 0.0044 mmol, 61% yield, TFA salt) as yellow gum.

Ex. 103 was prepared in a similar manner described in the synthesis of Ex. 102 from 102-2.

### Preparation of 2-methyl-4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol (Ex. 104)

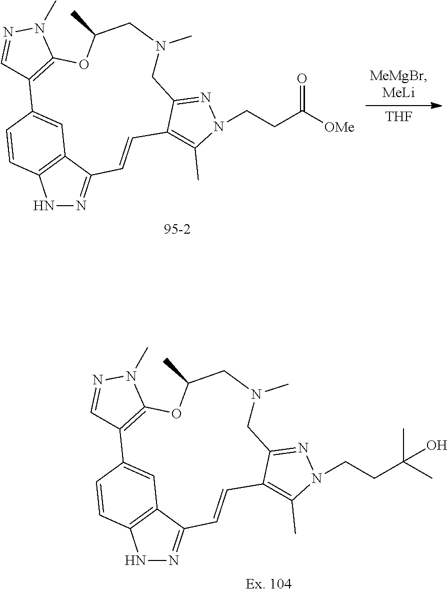 [SMILES: "THF" ; COC(=O)CCn1nc2c(c1C)/C=C/c1n[nH]c3ccc(cc13)-c1cnn(C)c1O[C@@H](C)CN(C)C2 ; Cc1c2c(nn1CCC(C)(C)O)CN(C)C[C@H](C)Oc1c(cnn1C)-c1ccc3[nH]nc(c3c1)/C=C/2]

Step 1. A mixture of methyl 3-[(8S,17E)-5,8,10,15-tetramethyl-7-oxa-4,5,10,13,14,20,21-heptazapentacyclo[17.5.2.0^{2,6}.0^{12,16}0.0^{22,26}]hexacosa-1(25),2(6),3,12,15,17,19,22(26), 23-nonaen-14-yl]propanoate (60 mg, 0.122 mmol, 1 eq) in THE (1 mL) was degassed and purged with N2 for 3 times, MeMgBr (3 M, 408 μL, 10 eq) and MeLi (4.8M, 76.6 uL, 1 eq) was added at 0° C., and then the mixture was stirred at 20° C. for 0.5 h under N2 atmosphere. On completion, the reaction mixture was quenched by addition sat. NH₄Cl 10 (mL) at 0° C., and then diluted with H₂O (30 mL) and extracted with EA 90 (3*30 mL). The combined organic layers were dried over Na₂SO₄, filtered and concentrated under reduced pressure to give a residue. The crude product was purified by reversed-phase HPLC (Welch Ultimate C18 150*25 mm*5 um; mobile phase: [water (TFA)-ACN |; B %: 10%-40%, 10 min) to give Ex. 104 (3.09 mg, 0.0062 mmol, 5% yield, 98% purity) as a brown gum.

### Preparation of 2-methyl-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol (Ex. 105)

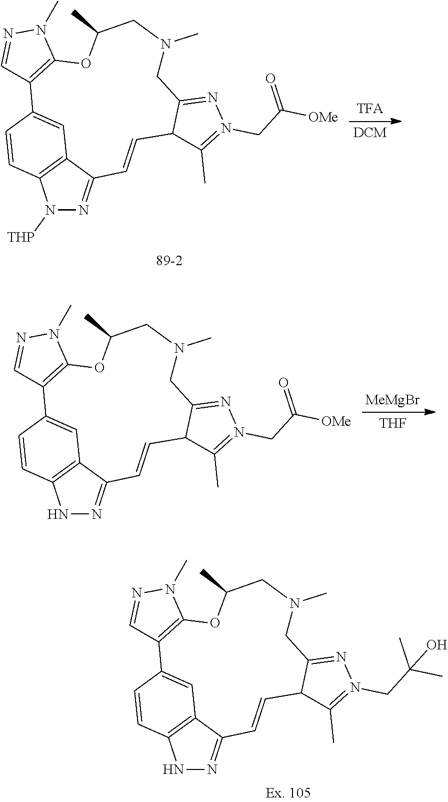

Step 1. To a mixture of methyl 2-[(8S,17E)-5,8,10,15-tetramethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6), 3,12,15,17,19,22(26),23-nonaen-14-yl]acetate (120 mg, 0.214 mmol, 1 eq) in DCM (1 mL) was added TFA (1.54 g, 13.5 mmol, 1 mL, 62.9 eq). The reaction mixture was stirred at 25° C. for 0.5 hour. On completion, the residue was diluted with water (20 mL) and extracted with EA (2×20 mL). The combined organic layers was dried over Na₂SO₄, filtered and concentrated in vacuo to give a residue. The residue was purified by prep-HPLC (column: Welch Xtimate C18 150*25 mm*5 um; mobile phase: [water(TFA)-ACN]; B %: 5%-35%,10 min) to afford methyl 2-[(8S,17E)-5,8,10,15-tetramethyl-7-oxa-4,5,10,13,14,20,21-heptazapentacyclo[17.5.2.0^{2,6}.0^{12,16}0.0^{22,26}]hexacosa-1(25),2(6),3,12,15,17,19,22(26), 23-nonaen-14-yl]acetate (80.0 mg, 0.168 mmol, 78% yield) as yellow solid. LCMS: m/z 476.4 (M+1)

Step 2. To a mixture of methyl 2-[(8S,17E)-5,8,10,15-tetramethyl-7-oxa-4,5,10,13,14,20,21-heptazapentacyclo[17.5.2.0^{2,6}.0^{12,16}0.0^{22,26}]hexacosa-1(25),2(6),3,12,15,17,19,22(26), 23-nonaen-14-yl]acetate (40.0 mg, 0.084 mmol, 1 eq) in THF (2 mL) was added MeMgBr (3 M, 140 μL, 5 eq). The reaction mixture was stirred at 25° C. for 4 hours. On completion, the residue was diluted with water (20 mL) and extracted with EA (2×20 mL). The combined organic layers was dried over Na₂SO₄, filtered and the filtrate was concentrated in vacuo to give a residue. The residue was purified by prep-HPLC (column: Phenomenex luna C18 150*25 mm*10 um; mobile phase: [water(FA)-ACN]; B %: 7%-27%, 9 min) to afford Ex. 105 (1.68 mg, 0.003 mmol, 3% yield, 97% purity, FA) as white solid.

### Preparation of (11S,17E)-8,10,11,14,16-pentamethyl-2,9,10,11,12,14-hexahydro-8H-3,5-ethenotripyrazolo[3,4-f:4′,3′-j:4″,3″-n][1,4]oxazacyclopentadecine (Ex. 106)

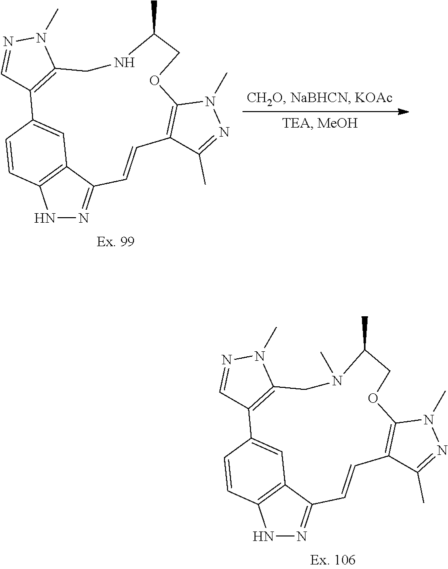

Step 1. To a solution of (9S,17E)-5,9,13,15-tetramethyl-11-oxa-4,5,8,13,14,20,21-heptazapentacyclo[17.5.2.0².0¹²0.1⁶0.0²²,2⁶]hexacosa-1(25),2(6),3,12(16),14,17,19, 22(26), 23-nonaene (25 mg, 0.062 mmol, 1 eq) in MeOH (1 mL) added TEA (18.8 mg, 0.186 mmol, 3 eq), and then formaldehyde (2.23 mg, 0.074 mmol, 1.2 eq), KOAc (12.2 mg, 0.124 mmol, 2 eq) was added. The mixture above was stirred at 25° C. for 30 minutes, then NaBH₃CN (4.67 mg, 0.074 mmol, 1.2 eq) was added. The mixture was stirred at 25° C. for 0.5 h. On completion, the reaction mixture was diluted with H₂O (50 mL) and extracted with EtOAc (20 mL×3). The combined organic layers were washed with brine 50 mL, dried over Na₂SO₄, filtered and concentrated under reduced pressure to give a residue. The residue was purified by Prep-HPLC (column: Phenomenex luna C18 150*25 mm*10 um; mobile phase: [water(FA)-ACN]; B %: 13%-43%,9 min) to give Ex. 106 (7.84 mg, 0.019 mmol, 30% yield, 95% purity) as white solid.

Ex. 107 and 108 were made during the synthesis of Ex. 102 and 103.

### Preparation of (10R,15E)-3,12,14-trimethyl-5,6,9,10,12,18-hexahydro-3H,8H-19,21-etheno-7,10-methanotripyrazolo[3,4-i:3′,4′-m:4″,3″-q][1,8,4]dioxazacyclooctadecin-24-one (Ex. 109)

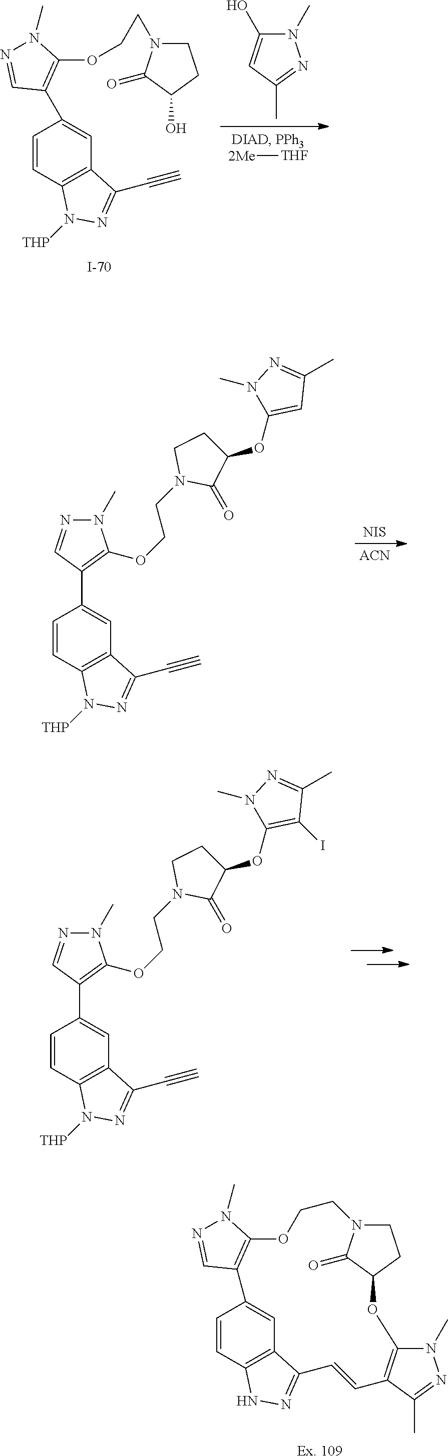

Step 1. The mixture of (3S)-1-[|2-|4-(3-ethynyl-1-tetrahydropyran-2-yl-indazol-5-yl)-2-methyl-pyrazol-3-yl]oxyethyl]-3-hydroxy-pyrrolidin-2-one (500 mg, 1.11 mmol, 1 eq) 2,5-dimethylpyrazol-3-ol (274.40 mg, 2.45 mmol, 11.04 uL, 2.2 eq) and PPh₃ (642 mg, 2.45 mmol, 2.2 eq) in 2-MeTHF (5 mL) was stirred at 25° C. for 30 min, then DIAD (495 mg, 2.45 mmol, 476 μL, 2.2 eq) was added dropwise to the mixture at 0° C., the mixture was stirred for another 2 h at 25° C. under N₂ atmosphere. On completion, the mixture was filtered and the filtrate was concentrated under reduced pressure to give a residue. The residue was purified by combi flash (12 g silica gel column, THE in PE from 0-100%) to give (3R)-3-(2,5-dimethylpyrazol-3-yl)oxy-1-[2-[4-(3-ethynyl-1-tetrahydropyran-2-yl-indazol-5-yl)-2-methyl-pyrazol-3-yl]oxyethyl]pyrrolidin-2-one (502 mg, 0.923 mmol, 83% yield) as a brown oil. LCMS: m/z 544.2 (M+1)

Step 2. To a solution of (3R)-3-(2,5-dimethylpyrazol-3-yl)oxy-1-[2-[4-(3-ethynyl-1-tetrahydropyran-2-yl-indazol-5-yl)-2-methyl-pyrazol-3-yl]oxyethyl]pyrrolidin-2-one (480 mg, 0.883 mmol, 1 eq) in ACN (15 mL) was added NIS (179 mg, 0.795 mmol, 0.9 eq) at 0° C. The mixture was stirred at 0° C. for 1 h. On completion, the reaction mixture was quenched with water saturated solution of Na₂SO₃ (30 mL) at 20° C., and extracted with EtOAc (20 mL*3). The combined organic layer was dried over anhydrous Na₂SO₄, filtered and the filtrate was concentrated in vacuum to give crude. The residue was purified by combi flash (12 g silica gel column, THF in PE from 0-100%) to give (3R)-1-[2-[4-(3-ethynyl-1-tetrahydropyran-2-yl-indazol-5-yl)-2-methyl-pyrazol-3-yl]oxyethyl]-3-(4-iodo-2,5-dimethyl-pyrazol-3-yl)oxy-pyrrolidin-2-one (395 mg, 0.590 mmol, 67% yield) as a brown oil. LCMS: m/z 670.1 (M+1)

The remaining steps were conducted in a manner similar to those described in General Method L to afford Ex. 109

Ex. 110 was prepared in a manner like those described in General Method N starting with step 6, with intermediates C-6-3 and 98-1. Then TFA/DCM in the final deprotection as described in General Method L.

### Preparation of (17E)-10-ethyl-8,14,16-trimethyl-2,9,10,11,12,14-hexahydro-8H-3,5-ethenotripyrazolo[3,4-f:4′,3′-j:4″,3″-n][1,4]oxazacyclopentadecine (Ex. 111)

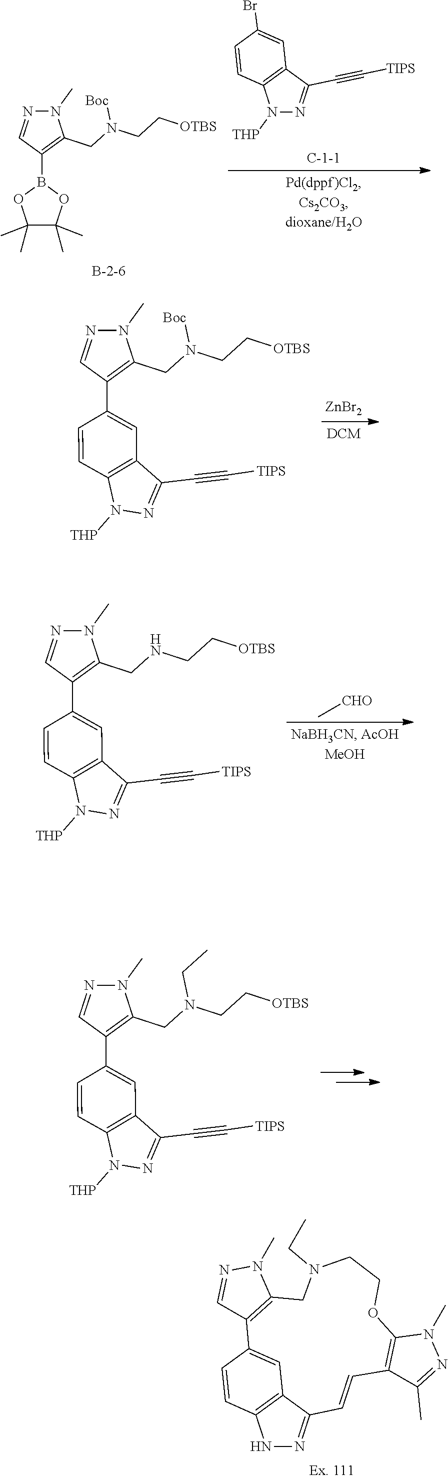

Step 1. A mixture of tert-butyl N-[2-[tert-butyl(dimethyl)silyl]oxyethyl]-N-[[2-methyl-4-(4,4,5,5-tetramethyl-1,3,2-dioxaborolan-2-yl)pyrazol-3-yl]methyl]carbamate, B-2-6 (5 g, 10.0 mmol, 1 eq), 2-(5-bromo-1-tetrahydropyran-2-yl-indazol-3-yl)ethynyl-triisopropyl-silane, C-1-1 (5.12 g, 11.1 mmol, 1.1 eq), Pd(dppf)Cl₂·CH₂Cl₂ (824 mg, 1.01 mmol, 0.1 eq), and Cs₂CO₃ (9.86 g, 30.2 mmol, 3 eq) in dioxane (100 mL) and H₂O (20 mL) was degassed and purged with N₂ for 3 times, and then the mixture was stirred at 80° C. for 2 h under N₂ atmosphere. On completion, the combined organic phase was dried over Na₂SO₄, filtered and the filtrate was concentrated to give a residue. The residue was purified by flash silica gel chromatography (ISCO®; 80 g SepaFlash® Silica Flash Column, Eluent of 0-50% THF/PE@70 mL/min) to give tert-butyl N-[2-[tert-butyl(dimethyl)silyl]oxyethyl]-N-[[2-methyl-4-[1-tetrahydropyran-2-yl-3-(2-triisopropylsilylethynyl) indazol-5-yl]pyrazol-3-yl]methyl]carbamate (4.09 g, 5.45 mmol, 54% yield) as a yellow oil. LCMS: m/z 750.5 (M+1)

Step 2. To a solution of tert-butyl N-[2-[tert-butyl(dimethyl)silyl]oxyethyl]-N-[[2-methyl-4-[1-tetrahydropyran-2-yl-3-(2-triisopropylsilylethynyl) indazol-5-yl]pyrazol-3-yl]methyl]carbamate (4.09 g, 5.45 mmol, 1 eq) in DCM (70 mL) was added ZnBr₂ (3.68 g, 16.3 mmol, 818 μL, 3 eq). The mixture was stirred at 25° C. for 16 h. On completion, the mixture was quenched by H₂O (150 mL) and extracted with DCM (70 mL*3). The combined organic layer was dried over anhydrous Na₂SO₄, filtered and the filtrate was concentrated in vacuum to give crude. The residue was purified by flash silica gel chromatography (ISCO®; 20 g SepaFlash® Silica Flash Column, Eluent of 0-50% THE/PE @50 mL/min) to give 2-[tert-butyl(dimethyl)silyl]oxy-N-[[2-methyl-4-[l-tetrahydropyran-2-yl-3-(2-triisopropylsilylethynyl) indazol-5-yl]pyrazol-3-yl]methyl]ethanamine (1 g, 1.54 mmol, 28% yield) as a yellow oil. LCMS: m/z 650.4 (M+1)

Step 3. To a solution of 2-[tert-butyl(dimethyl)silyl]oxy-N-[[2-methyl-4-[1-tetrahydropyran-2-yl-3-(2-triisopropylsilylethynyl) indazol-5-yl]pyrazol-3-yl]methyl]ethanamine (1 g, 1.54 mmol, 1 eq) and acetaldehyde (203 mg, 1.85 mmol, 258 μL, 40% purity, 1.2 eq) in MeOH (15 mL) was added NaBH₃CN (116 mg, 1.85 mmol, 1.2 eq). The reaction mixture was stirred at 25° C. for 1 h. On completion, the mixture was concentrated to give a residue. The residue was purified by flash silica gel chromatography (ISCO®; 12 g SepaFlash® Silica Flash Column, Eluent of 0-50% THF/PE @40 mL/min) to give 2-[tert-butyl(dimethyl)silyl]oxy-N-ethyl-N-[[2-methyl-4-[1-tetrahydropyran-2-yl-3-(2-triisopropylsilylethynyl) indazol-5-yl]pyrazol-3-yl]methyl]ethanamine (1.18 g, crude) as a yellow oil. LCMS: m/z 678.4 (M+1) [01427]2-[tert-butyl(dimethyl)silyl]oxy-N-ethyl-N—[[2-methyl-4-[1-tetrahydropyran-2-yl-3-(2-triisopropylsilylethynyl) indazol-5-yl]pyrazol-3-yl]methyl]ethanamine was converted to Ex. 111 via processes similar to those described in General Method N using TFA/DCM in the final deprotection.

### Preparation of tert-butyl 4-(3-hydroxy-4-iodo-phenyl)piperidine-1-carboxylate (I-112)

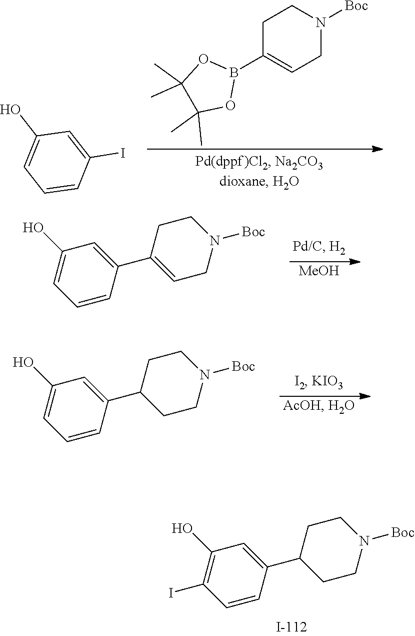

Step 1. To a solution of 3-bromophenol (2 g, 11.6 mmol, 1 eq) in dioxane (15 mL) and H₂O (2.1 mL) was added Pd(dppf)Cl (846 mg, 1.16 mmol, 0.1 eq) and Na₂CO₃ (3.68 g, 34.7 mmol, 3 eq) and tert-butyl 4-(4,4,5,5-tetramethyl-1,3,2-dioxaborolan-2-yl)-3,6-dihydro-2H-pyridine-1-carboxylate (3.57 g, 11.6 mmol, 1 eq). The mixture was stirred at 80° C. for 2 h On completion, the residue was diluted with H₂O 80 mL and extracted with EA (80 mL*3), dried over Na₂SO₄, filtered and concentrated under reduced pressure to give a residue. The residue was purified by column chromatography (SiO₂, Petroleum ether/Ethyl acetate=1/0 to 4/1) to give tert-butyl 4-(3-hydroxyphenyl)-3,6-dihydro-2H-pyridine-1-carboxylate (3 g, 10.90 mmol, 94% yield) as a black solid. LCMS: m/z 220.0 (M−56+1)

Step 2. A mixture of tert-butyl 4-(3-hydroxyphenyl)-3,6-dihydro-2H-pyridine-1-carboxylate (3.46 g, 12.57 mmol, 1 eq), Pd/C (0.35 g, 10% purity) in MeOH (40 ml) was degassed and purged with H₂ for 3 times, and then the mixture was stirred at 25° C. for 2 h under H₂ atmosphere. On completion, the reaction mixture was filtrated and the filtrated was concentrated in vacuum to give tert-butyl 4-(3-hydroxyphenyl)piperidine-1-carboxylate (3.45 g, 12.4 mmol, 99% yield) as brown oil. LCMS: m/z 222.0 (M−56+1)

Step 3. To a solution of tert-butyl 4-(3-hydroxyphenyl)piperidine-1-carboxylate (3 g, 10.8 mmol, 1 eq) in H₂O (5 mL) and AcOH (20 mL) was added I₂ (1.37 g, 5.41 mmol, 1.09 mL, 0.5 eq) and 2-iodopropane (368 mg, 2.16 mmol, 216 μL, 0.2 eq).The mixture was stirred at 25° C. for 2 h. On completion, the residue was diluted with H₂O 70 mL and extracted with EA (70 mL*3). The combined organic layers were washed with H₂O (20 mL*3), dried over Na₂SO₄, filtered and concentrated under reduced pressure to give a residue. The residue was purified by column chromatography (SiO₂, Petroleum ether/Ethyl acetate=1/0 to 5/1) to give tert-butyl 4-(3-hydroxy-4-iodo-phenyl)piperidine-1-carboxylate I-112 (2.02 g, 5.01 mmol, 46% yield) as a white solid. LCMS: m/z 348.0 (M−56+1)

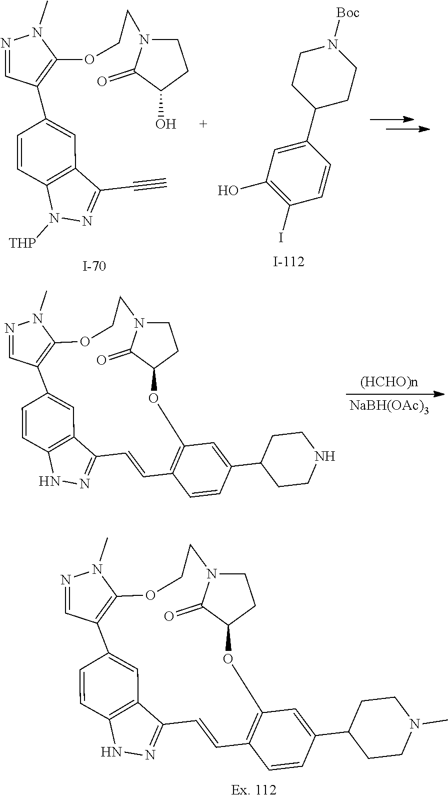

Step 1. 1-112 and 1-70 was converted to (13R,21E)-5-methyl-17-(4-piperidyl)-7,14-dioxa-4,5,10,24,25-pentazahexacyclo[21.5.2.110,13.02,6.015,20.026, 30]hentriaconta-1(29),2(6),3,15(20),16,18,21,23,26(30),27-decaen-31-one using process described in General Method L.

Step 2. To a solution of (13R,21E)-5-methyl-17-(4-piperidyl)-7,14-dioxa-4,5,10,24,25-pentazahexacyclo[21.5.2.110,13.02,6.015,20.026,30]hentriaconta-1(29),2(6),3,15(20),16,18,21,23,26(30),27-decaen-31-one (40 mg, 0.076 mmol, 1 eq) in MeOH (4 mL) was added KOAc (150 mg, 1.52 mmol, 20 eq) and NaBH(OAc)₃ (32.3 mg, 0.152 mmol, 2 eq). The mixture was stirred at 20° C. for 0.5 h, then HCHO (11.4 mg, 0.381 mmol, 10.5 uL, 5 eq) was added, the mixture was stirred at 20° C. for 0.5 h. On completion, the mixture was filtered and the filtrate was concentrated under reduced pressure to give a residue. The residue was purified by prep-HPLC (column: Phenomenex luna C18 150*25 mm*10 um; mobile phase: [water(FA)-ACN]; B %: 7%-37%,10 min) to give Ex. 112 (3.69 mg, 0.007 mmol, 9% yield, 100% purity) as a yellow solid.

### Preparation of (10S,17E)-8,10,12,16-tetramethyl-2,10,11,12,13,16-hexahydro-8H-3,5-ethenotripyrazolo[4,3-f:3′,4′-j:4″,3″-n][1,4]oxazacyclopentadecine (Ex. 113)

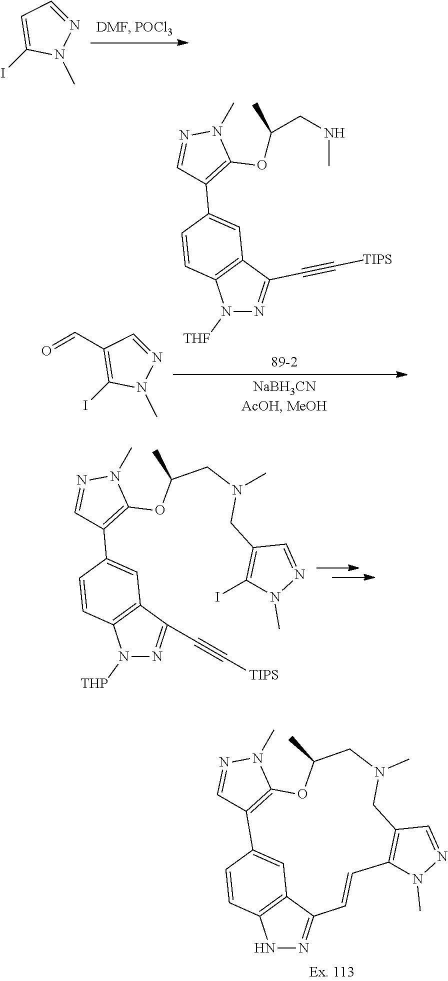

Step 1. The solution of 5-iodo-1-methyl-pyrazole (5.00 g, 24.0 mmol, 1 eq) in DMF (25 mL) was added POCl₃ (11.0 g, 72.1 mmol, 3 eq) at 0° C. The mixture was stirred at 25° C. for 4 h. On completion, the solution was added to sat. K₂CO₃ aqueous solution (7 mL), the mixture was extracted with EA (50 mL*2), the organic layer was washed with brine (10 mL), dried over Na₂SO₄, filtered and concentrated to give a residue. The residue was purified by column chromatography (SiO₂, PE/THF=3:1 to 3:1) to give 5-iodo-1-methyl-pyrazole-4-carbaldehyde (1.18 g, 5.00 mmol, 21% yield) as a yellow solid. LCMS: m/z 236.9 (M+1)

Step 2. The solution of 5-iodo-1-methyl-pyrazole-4-carbaldehyde (141 mg, 0.600 mmol, 1 eq) in MeOH (5 mL) was added 89-2 (330 mg, 0.600 mmol, 1 eq) and AcOH (36.0 mg, 0.600 mmol, 1 eq), the resulting mixture was stirred at 25° C. for 30 min, then NaBH₃CN (75.4 mg, 1.20 mmol, 2 eq) was added at 0° C. The mixture was stirred at 25° C. for 12 hr. On completion, the reaction mixture was quenched by addition water 10 mL. The mixture was concentrated to give a residue. The residue was purified by column chromatography (SiO₂, PE/THF=1:1 to 1:1) to give (2S)—N-[(5-iodo-1-methyl-pyrazol-4-yl)methyl]-N-methyl-2-[2-methyl-4-[1-tetrahydropyran-2-yl-3-(2-triisopropylsilylethynyl) indazol-5-yl]pyrazol-3-yl]oxy-propan-1-amine (210 mg, 0.272 mmol, 45% yield) as a yellow solid. LCMS: m/z 770.1 (M+1)

The remaining steps was conducted similarly to those detailed in General Method H to afford Ex. 113.

### Preparation of methyl 2-[5-(bromomethyl)-4-iodo-3-methyl-pyrazol-1-yl]acetate (I-114)

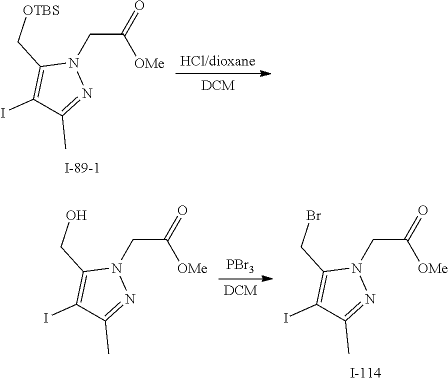

I-114 was made similarly to I-89 from I-89-2. 1H NMR (400 MHz, DMSO-d6) δ 5.13 (s, 2H), 4.70 (s, 2H), 3.67 (s, 3H), 2.11 (s, 3H).

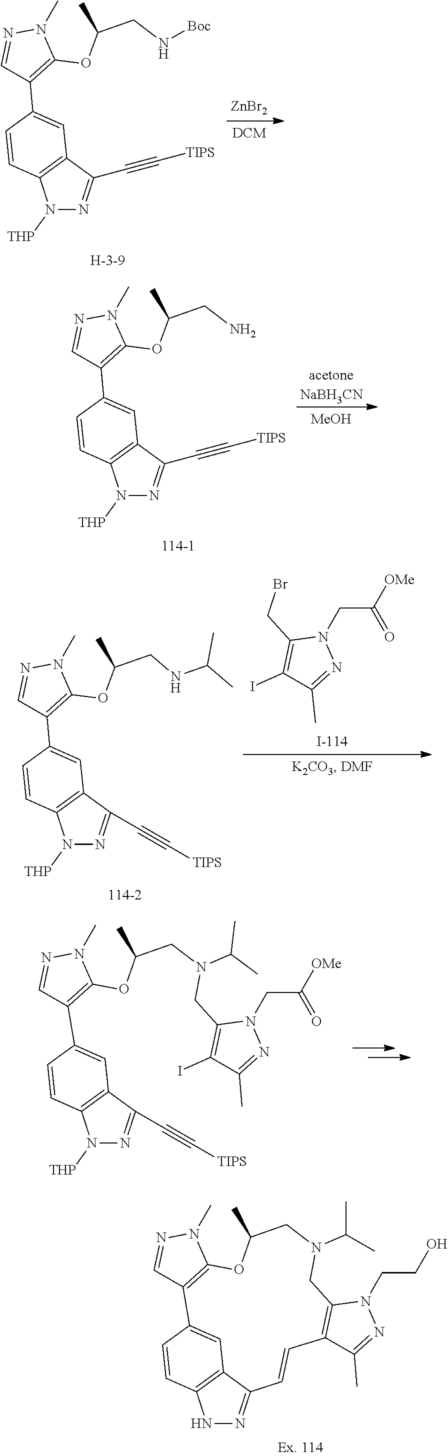

Step 1. To a mixture of H-3-9 (6.56 g, 10.3 mmol, 1 eq) in DCM (80 mL) was added ZnBr₂ (11.6 g, 51.5 mmol, 5 eq), the reaction mixture was stirred at 25° C. for 2 hours. On completion, the reaction mixture was concentrated in vacuo and then the residue was diluted with water (100 mL) and extracted with EA (2×100 mL). The combined organic layers was dried over Na₂SO₄, filtered and concentrated in vacuo to afford compound (2S)-2-[2-methyl-4-[l-tetrahydropyran-2- yl-3-(2-triisopropylsilylethynyl) indazol-5-yl]pyrazol-3-yl]oxypropan-1-amine (5.80 g, 90% yield) as brown solid. ¹H NMR (400 MHz, DMSO-d₆) δ 7.82 (d, J=8.8 Hz, 1H), 7.75 (s, 1H), 7.71 (d, J=1.2 Hz, 1H), 7.69 (s, 1H), 5.88 (d, J=9.6 Hz, 1H), 4.23 (td, J=6.0, 12.0 Hz, 1H), 4.15 (dt, J=2.8, 5.6 Hz, 1H), 4.05-4.00 (m, 2H), 3.92 (s, 1H), 3.89 (s, 1H), 3.72 (s, 3H), 2.88-2.79 (m, 1H), 2.77-2.70 (m, 1H), 2.41-2.31 (m, 1H), 2.03 (d, J=4.0 Hz, 1H), 1.99 (s, 3H), 1.79-1.69 (m, 1H), 1.59 (s, 2H), 1.19 (s, 1H), 1.17 (d, J=1.6 Hz, 2H), 1.15 (s, 18H)

Step 2. To a mixture of (2S)-2-[2-methyl-4-[1-tetrahydropyran -2-yl-3-(2-triisopropylsilylethynyl) indazol-5-yl]pyrazol-3-yl]oxypropan-1-amine (4.14 g, 7.73 mmol, 1 eq) in MeOH (40 mL) was added acetone (1.35 g, 23.1 mmol, 1.70 mL, 3 eq), the reaction mixture was stirred at 25° C. for 0.5 hour, and then NaBH₃CN (728 mg, 11.5 mmol, 1.5 eq) was added to the reaction mixture, the reaction mixture was stirred at 25° C. for 1 hour. On completion, the residue was diluted with water (50 mL) and extracted with EA (2×50 mL). The combined organic layers was dried over Na₂SO₄, filtered and concentrated in vacuo to afford compound (2S)—N—isopropyl -2-[2-methyl-4-[1-tetrahydropyran-2-yl-3-(2-triisopropylsilylethynyl) indazol-5-yl]pyrazol-3-yl]oxy-propan-1-amine (3.29 g, 5.69 mmol, 73% yield) as yellow oil. LCMS: m/z 578.3 (M+1) Step 3. To a mixture of (2S)—N—isopropyl -2-[2-methyl-4-[1-tetrahydropyran-2- yl-3- (2-triisopropylsilylethynyl) indazol-5-yl]pyrazol-3-yl]oxy-propan-1-amine (1.15 g, 1.99 mmol, 1 eq) in DMF (30 mL) was added K₂CO₃ (825 mg, 5.97 mmol, 3 eq) and I-114 (2.97 g, 7.96 mmol, 4 eq), the mixture was stirred at 25° C. for 12 hours. On completion, the residue was diluted with water (50 mL) and extracted with EA (2×50 mL). The combined organic layers was dried over Na₂SO₄, filtered and concentrated in vacuo to give a residue. The residue was purified by column chromatography to afford compound methyl 2-[4-iodo-5-[[isopropyl-[(2S)-2-[2-methyl-4-[1-tetrahydropyran-2-yl-3-(2-triisopropylsilylethynyl) indazol-5-yl]pyrazol-3-yl]oxypropyl]amino]methyl]-3-methyl-pyrazol-1-yl]acetate (1.53 g, 1.76 mmol, 88% yield) as yellow oil. 1H NMR (400 MHz, DMSO-d6) δ 7.95 (s, 2H), 7.82 (s, 1H), 7.61 (d, J=8.8 Hz, 1H), 5.88 (dd, J=2.4, 9.6 Hz, 2H), 5.75 (s, 1H), 4.03 (q, J=7.2 Hz, 1H), 3.60 (d, J=0.8 Hz, 3H), 3.50 (s, 2H), 3.49 (s, 3H), 3.44-3.42 (m, 2H), 2.38-2.31 (m, 2H), 2.12 (s, 1H), 2.09 (d, J=4.0 Hz, 1H), 2.05 (s, 3H), 1.99 (s, 4H), 1.95-1.90 (m, 1H), 1.59 (d, J=3.2 Hz, 3H), 1.24 (s, 3H), 1.16-1.16 (m, 6H), 1.15 (s, 18H).

The remaining steps were conducted similarly to those described in General Method M to afford Ex. 114.

### Preparation of (10R,16E)-13-(2-methoxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one (Ex. 115)

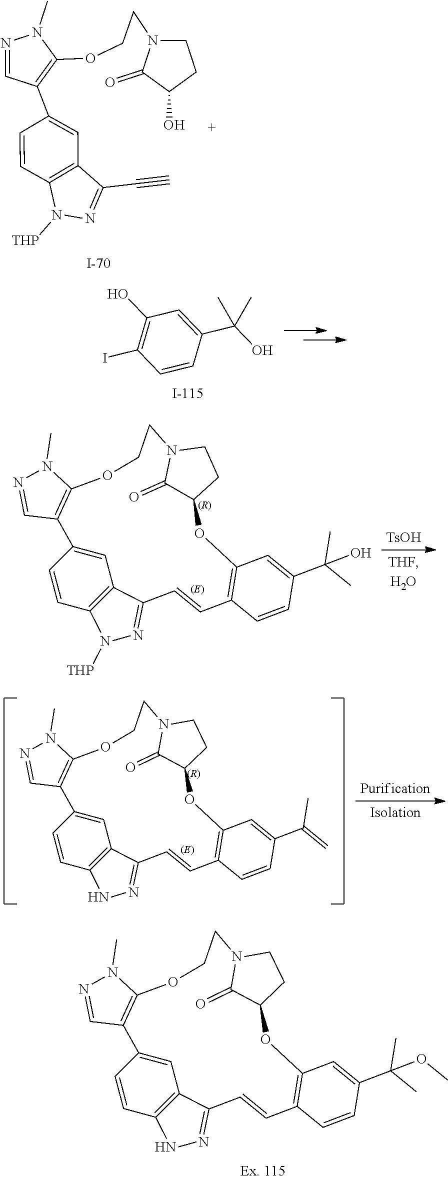

I-115 was prepared similarly to those described in General Method E.

I-70 and I-115 were converted to (13R,21E)-17-(1-hydroxy-1-methyl-ethyl)-5-methyl-25-tetrahydropyran-2-yl-7,14-dioxa-4,5,10,24,25-pentazahexacyclo[21.5.2.110,13.02,6.015,20.026,30]hentriaconta-1(29),2(6),3,15(20),16,18,21,23,26(30),27-decaen-31-one in a manner similar to those described in General Method L.

To a solution of (13R,21E)-17-(1-hydroxy-1-methyl-ethyl)-5-methyl-25-tetrahydropyran-2-yl-7,14-dioxa-4,5,10,24,25-pentazahexacyclo [21.5.2.110,13.02,6.015,20.026,30]hentriaconta-1(29),2(6),3,15(20), 16,18,21,23,26(30),27-decaen-31-one (50.0 mg, 0.086 mmol, 1 eq) in THF (1 mL) and H₂O (0.2 mL) was added TsOH (22.1 mg, 0.129 mmol, 1.5 eq). The mixture was stirred at 70° C. for 1 h. On completion, the mixture was filtered and concentrated under reduced pressure to give a residue. The residue was purified by prep-HPLC (column: Phenomenex luna C18 150*25 mm*10 um; mobile phase: [water(FA)-ACN]; B %: 33%-63%, 58 min) to give Ex. 115 (10.06 mg, 0.019 mmol, 22% yield, 98.3% purity) as a white solid.

Ex. 116 was prepared similarly to those described in General Method M from intermediate C-1-2, 89-1, and I-114.

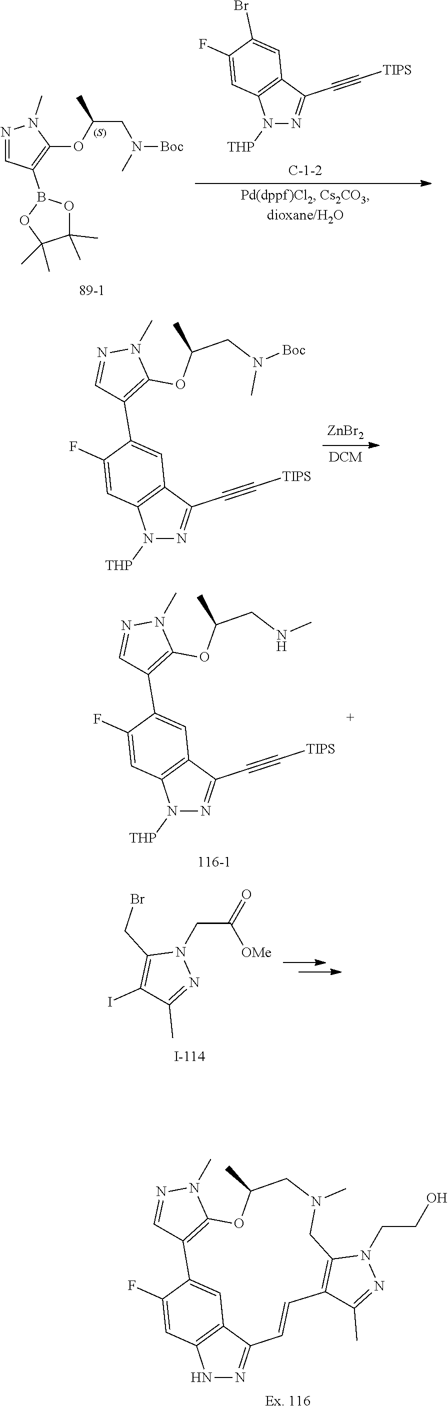

### Preparation of (17E)-8,10,16-trimethyl-14-(1-methylpyrrolidin-3-yl)-2,9,10,11,12,14-hexahydro-8H-3,5-ethenotripyrazolo[3,4-f:4′,3′-j:4″,3″-n][1,4]oxazacyclopentadecine (Ex. 117) and (17E)-8,10,16-trimethyl-14-(1-methylpyrrolidin-3-yl)-2,9,10,11,12,14-hexahydro-8H-3,5-ethenotripyrazolo[3,4-f:4′,3′-j:4″,3″-n][1,4]oxazacyclopentadecine (Ex. 118)

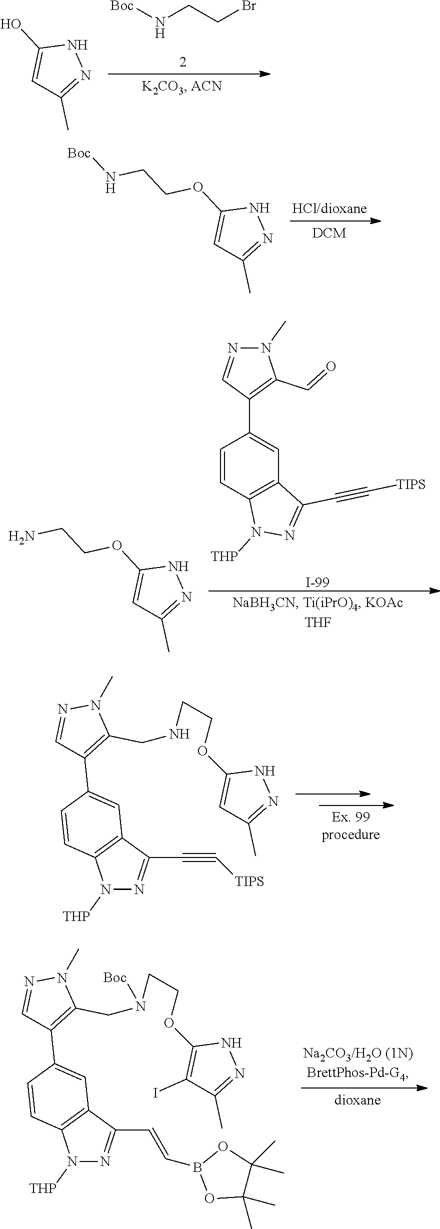

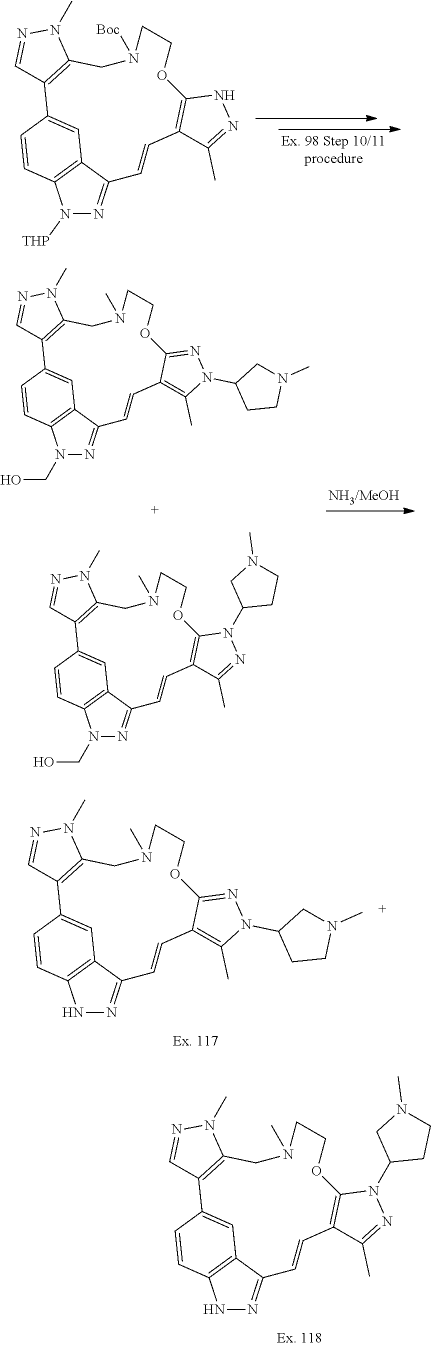

Step 1. To a mixture of 3-methyl-1H-pyrazol-5-ol (3.00 g, 30.5 mmol, 1 eq), tert-butyl N-(2-bromoethyl)carbamate (6.85 g, 30.5 mmol, 1 eq) in ACN (60 mL) was added K₂CO₃ (12.6 g, 91.7 mmol, 2 eq). The mixture was stirred at 100° C. for 4 hours. On completion, the mixture was poured into the water (50 mL), extracted with EA (40 mL*3), the combined organic layers were washed with brine (50 mL*2), dried over Na₂SO₄, filtered and concentrated under reduced pressure to give a residue. The residue was purified by column chromatography (SiO₂, Petroleum ether/Ethyl acetate=10/1 to 2/1) to give tert-butyl N-[2-[(3-methyl-1H-pyrazol-5-yl)oxy]ethyl]carbamate (3.60 g, 48% yield) as a brown oil. ¹H NMR (400 MHz, DMSO-d₆) δ=11.52 (s, 1H), 6.94 (t, J=5.2 Hz, 1H), 5.41 (s, 1H), 3.98-3.93 (m, 2H), 3.27-3.19 (m, 3H), 2.13 (s, 3H), 1.38 (s, 9H).

Step 2. To a mixture of tert-butyl N-[2-[(3-methyl-1H-pyrazol-5-yl)oxy]ethyl]carbamate (1.50 g, 6.22 mmol, 1 eq) in DCM (10 mL) was added HCl/dioxane (4 M, 3 mL) and the mixture was stirred at 20° C. for 10 minutes. On completion, the mixture was concentrated under reduced pressure to give 2-[(3-methyl-1H-pyrazol-5-yl)oxy]ethanamine (870 mg, 78% yield, HCl) as a brown oil.

Step 3. To a mixture of 2-[(3-methyl-1H-pyrazol-5-yl)oxy]ethanamine (870 mg, 4.90 mmol, 1 eq, HCl), 2-methyl-4-[1-tetrahydropyran-2-yl-3-(2-triisopropylsilylethynyl) indazol-5-yl]pyrazole-3-carbaldehyde (1.92 g, 3.92 mmol, 0.8 eq), KOAc (4.81 g, 48.9 mmol, 10 eq) in THF (30 mL) was added Ti(i-PrO)₄ (6.96 g, 24.4 mmol, 5 eq). The mixture was stirred at 80° C. for 11.5 hours under N₂ atmosphere. Then the mixture was cooled to 25° C. NaBH₃CN (615 mg, 9.80 mmol, 2 eq) was added into the mixture and stirred for 0.5 hours at 25° C. On completion, the reaction mixture was poured into H₂O (100 mL) and stirred for 10 minutes, filtered to give the mother liquor. The mother liquor was extracted with EtOAc (100 mL*3). The combined organic layers were washed with brine (100 mL*2), dried over Na₂SO₄, filtered and concentrated under reduced pressure to give a residue. The residue was purified by column chromatography (SiO₂, PE/EA=8:1 to 4:1) to give 2-[(3-methyl-1H-pyrazol-5-yl)oxy]-N-[[2-methyl-4-[1-tetrahydropyran-2-yl-3-(2-triisopropylsilylethynyl) indazol-5-yl]pyrazol-3-yl]methyl]ethanamine (1.20 g, 39% yield) as a brown oil. ¹H NMR (400 MHz, CDCl₃) δ=7.58 (s, 1H), 7.54 (d, J=8.8 Hz, 1H), 7.50 (s, 1H), 7.40-7.33 (m, 1H), 5.69-5.64 (m, 1H), 4.03 (d, J=3.6 Hz, 1H), 3.93 (s, 3H), 3.73-3.65 (m, 1H), 3.31 (t, J=7.2 Hz, 2H), 2.86-2.81 (m, 2H), 2.77 (s, 3H), 2.30 (t, J=8.0 Hz, 2H), 2.12 (s, 3H), 2.04 (d, J=8.8 Hz, 1H), 1.97-1.90 (m, 2H), 1.74-1.66 (m, 2H), 1.64-1.56 (m, 1H), 1.12-1.07 (m, 21H).

Steps 4-8 were conducted similarly to those described for Ex 99.

Step 9. To a mixture of tert-butyl N-[2-[(4-iodo-3-methyl-1H-pyrazol-5-yl)oxy]ethyl]-N-[[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]methylicarbamate (500 mg, 0.614 mmol, 1 eq), a solution of Na₂CO₃ in water (1 N, 1.2 mL, 3 eq), dicyclohexyl-[3,6-dimethoxy-2-(2,4,6-triisopropylphenyl)phenyl]phosphane;methanesulfonate;[2-[2-(methylamino) phenyl]phenyl]palladium(1+) (56.5 mg, 0.061 mmol, 0.1 eq) in dioxane (3 mL) was degassed and purged with N₂ for 3 times. The mixture was stirred at 80° C. for 2 hours under N₂ atmosphere. On completion, the mixture was concentrated to give the residue. The residue was purified by column chromatography (SiO₂, Petroleum ether/Ethyl acetate=5:1 to 2:1) to give the compound tert-butyl (17E)-5,15-dimethyl-21-tetrahydropyran-2-yl-11-oxa-4,5,8,13,14,20,21-heptazapentacyclo[17.5.2.0^{2,6}.0^{12,16}. 0^{22,26}]hexacosa-1(25),2(6),3,12(16),14,17,19,22(26),23-nonaene-8-carboxylate (189 mg, 54% yield) as a brown oil. ¹H NMR (400 MHz, CDCl₃) δ=7.63-7.59 (m, 3H), 6.91 (s, 1H), 5.66-5.58 (m, 1H), 5.00 (s, 2H), 4.37 (s, 2H), 4.08-3.96 (m, 1H), 3.81 (s, 3H), 3.71-3.65 (m, 6H), 3.62 (d, J=5.2 Hz, 2H), 2.58-2.47 (m, 1H), 2.41 (s, 3H), 2.15-1.98 (m, 3H), 1.36 (s, 9H).

Steps 10 and 11 were conducted in a manner similar to those described for Ex. 98, steps 10-11.

Step 12. A mixture of (17E)-5,8,15-trimethyl-13-(1-methylpyrrolidin-3-yl)-11-oxa-4,5,8,13,14, 20,21-heptazapentacyclo[17.5.2.0^{2,6}.0^{12,16}0.0^{22,26}]hexacosa-1(25),2(6),3,12(16),14,17, 19,22(26),23-nonaene (4.00 mg, 0.0085 mmol, 1 eq) in NH₃/MeOH (2 M, 2 mL) was stirred at 20° C. for 0.5 hours. On completion, the mixture was concentrated to give the residue. The residue was purified by prep-HPLC (column: Welch Xtimate C18 150*25 mm*5 um; mobile phase: [water (FA)-ACN]; B %: 20%-50%,min) to give Ex. 117 (2.84 mg, 64% yield, FA) as a gum brown.

Ex. 118 was obtained in the same fashion as Ex. 117

### General Method O. Preparation of 2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 119)

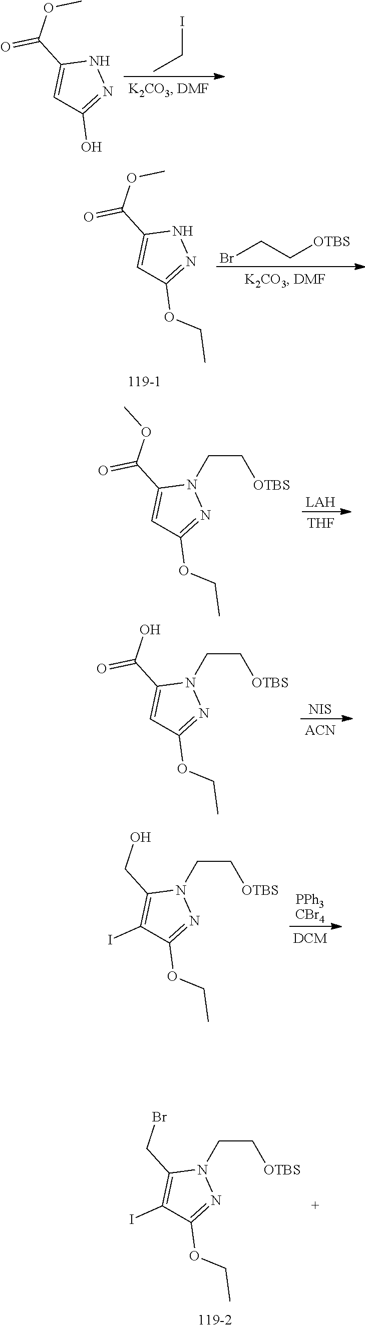

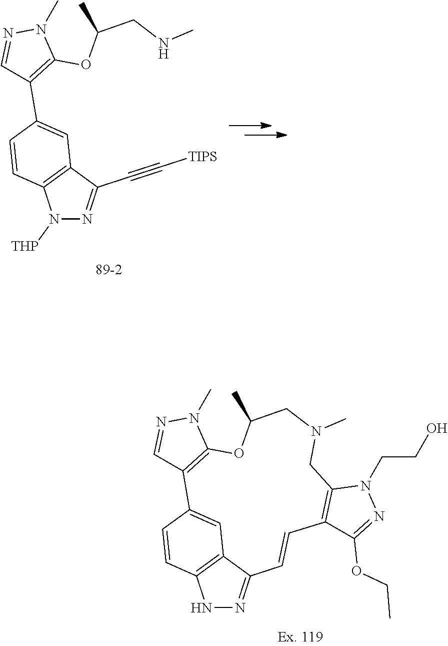

Step 1. To a solution of methyl 3-hydroxy-1H-pyrazole-5-carboxylate (15.0 g, 105 mmol, 1 eq), iodoethane (16.4 g, 105 mmol, 8.44 mL, 1 eq) in DMF (150 mL) was added K₂CO₃ (43.7 g, 316 mmol, 3 eq). The mixture was stirred at 80° C. for 2 hr. On completion, the mixture was diluted with H₂O (100 mL) and extracted with ethyl acetate (50 mL, ×3), the combined organic phase was washed with sat. NaCl (200 mL*2) and dried over Na₂SO₄, filtered and the filtrate was concentrated to give a residue. The residue was purified by column chromatography (SiO₂, Petroleum ether/Ethyl acetate=1:0 to 80:20) to give methyl 3-ethoxy-1H-pyrazole-5-carboxylate (10.4 g, 61.1 mmol, 58% yield) as a white solid. LCMS: m/z 171.1 (M+1)

Step 2. To a solution of methyl 3-ethoxy-1H-pyrazole-5-carboxylate (10.0 g, 58.7 mmol, 1 eq), 2-bromoethoxy-tert-butyl-dimethyl-silane (21.0 g, 88.1 mmol, 1.5 eq) in DMF (100 mL) was added NaI (8.81 g, 58.7 mmol, 1 eq), K₂CO₃ (24.3 g, 176 mmol, 3 eq). The mixture was stirred at 60° C. for 16 hr. On completion, the mixture was diluted with H₂O (100 mL) and extracted with ethyl acetate (40 mL×3). The combined organic phase was washed with sat. NaCl (80 mL*2) and dried over Na₂SO₄, filtered and the filtrate was concentrated to give a residue. The residue was purified by column chromatography (SiO₂, Petroleum ether/Ethyl acetate=1:0 to 17:83) to give methyl 1-(2-((tert-butyldimethylsilyl)oxy)ethyl)-3-ethoxy-1H-pyrazole-5-carboxylate (12.7 g, 38.6 mmol, 66% yield) as a yellow liquid. LCMS: m/z 329.5 (M+1)

Step 3. To a solution of methyl 2-[2-[tert-butyl(dimethyl)silyl]oxyethyl]-5-ethoxy-pyrazole-3-carboxylate (12.0 g, 36.5 mmol, 1 eq) in THF (120 mL) was added LiAlH₄ (1.39 g, 36.5 mmol, 1 eq) at 0° C. The mixture was stirred at 0° C. for 2 hr. On completion, the mixture was quenched with MeOH (150 mL) at 0° C., and the mixture was concentrated to give a residue. The residue was purified by column chromatography (SiO₂, Petroleum ether/Ethyl acetate=1:0 to 75:25) to give (1-(2-((tert-butyldimethylsilyl)oxy)ethyl)-3-ethoxy-1H-pyrazol-5-yl)methanol (12.7 g, 38.6 mmol, 66% yield) as a yellow liquid. LCMS: m/z 301.1 (M+1)

Step 4. To a solution of [2-[2-[tert-butyl(dimethyl)silyl]oxyethyl]-5-ethoxy-pyrazol-3-yl]methanol (7.00 g, 23.3 mmol, 1 eq) in ACN (70 mL) was added NIS (4.72 g, 20.9 mmol, 0.9 eq) at 0° C. The mixture was stirred at 25° C. for 2 hr. On completion, the mixture was quenched with sat. Na₂SO₃ (200 mL) at 0° C. and extracted with ethyl acetate (50 mL×3), the combined organic phase was dried over anhydrous Na₂SO₄, filtered and the filtrate was concentrated to give a residue. The residue was purified by column chromatography (SiO₂, Petroleum ether/Ethyl acetate=1:0 to 80:20) to give (1-(2-((tert-butyldimethylsilyl)oxy)ethyl)-3-ethoxy-4-iodo-1H-pyrazol-5-yl)methanol (7.70 g, 18.0 mmol, 66% yield) as a yellow liquid. ¹H NMR (400 MHz, CDCl₃) δ=4.60 (d, J=2.0 Hz, 2H), 4.30-4.25 (m, 2H), 4.23 (dt, J=2.4, 4.8 Hz, 2H), 3.95-3.90 (m, 2H), 3.33 (br s, 1H), 1.45-1.39 (m, 3H), 0.84 (d, J=2.0 Hz, 9H), 0.02-0.01 (m, 6H). LCMS: m/z 427.0 (M+1)

Step 5. To a solution of [2-[2-[tert-butyl(dimethyl)silyl]oxyethyl]-5-ethoxy-4-iodo-pyrazol-3-yl]methanol (3.50 g, 8.21 mmol, 1 eq), PPh₃ (2.58 g, 9.85 mmol, 1.2 eq) in DCM (35 mL) was added CBr₄ (3.27 g, 9.85 mmol, 1.2 eq) at 0° C. The mixture was stirred at 25° C. for t hr. On completion, the mixture was diluted with water (50 mL) and extracted with DCM (30 mL×3). The combined organic phase was dried over Na₂SO₄, filtered and the filtrate was concentrated to give a residue. The residue was purified by column chromatography (SiO₂, Petroleum ether/Ethyl acetate=1:0 to 80:20) to give 5-(bromomethyl)-1-(2-((tert-butyldimethylsilyl)oxy)ethyl)-3-ethoxy-4-iodo-1Hpyrazole, 119-2. (2.90 g, 5.93 mmol, 72% yield) as a yellow oil. LCMS: m/z 490.8 (M+1)

The remaining steps were conducted similarly to General Method H to give Ex. 119.

Ex. 120 was prepared in a manner similar to those described in General Method H using 89-2 in step 2.

Ex. 121 was prepared following procedures described in General Method M using tert-butyl N-[(2S)-2-hydroxypropyl]carbamate in step 1.

### Preparation of (10R,19E)-8,16,18-trimethyl-2,8,10,11,12,13,15,16-octahydro-3,5-etheno-10,14-methanotripyrazolo[3,4-h:3′,4′-l:4″,3″-p][1,6]oxazacycloheptadecine (Ex. 122)

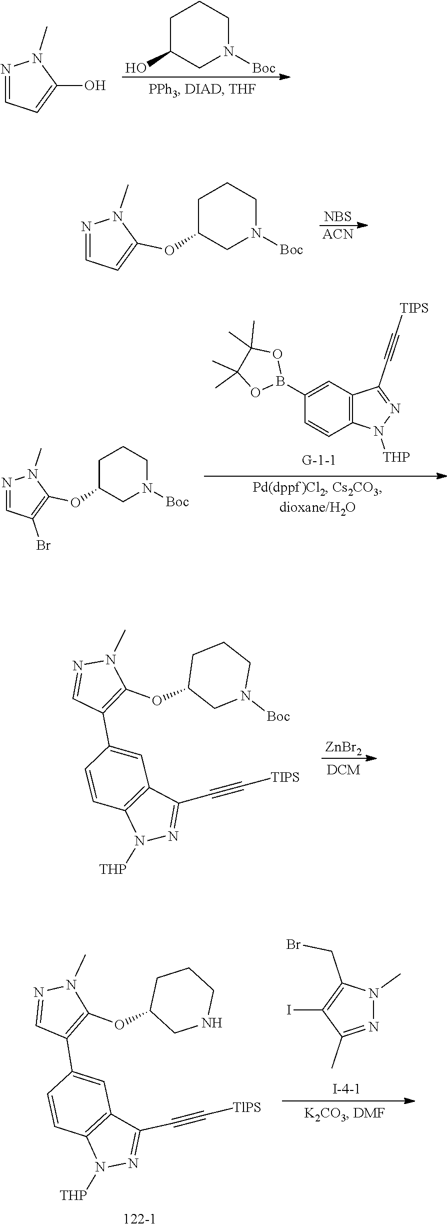

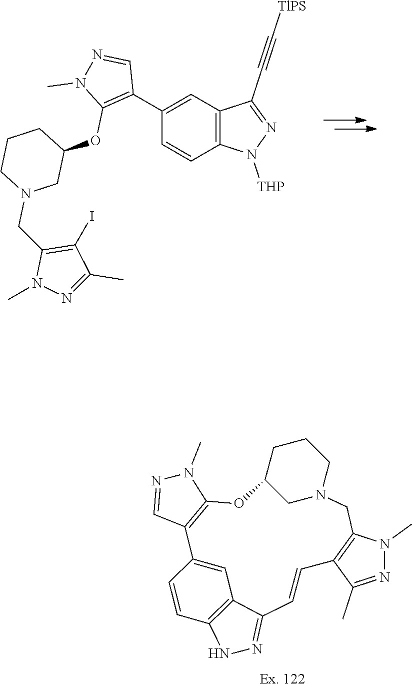

Step 1. To a solution of 2-methylpyrazol-3-ol (5.0 g, 50.9 mmol, 1.0 eq) and tert-butyl (3S)-3-hydroxypiperidine-1-carboxylate (15.4 g, 76.5 mmol, 1.5 eq), PPh₃ (16.0 g, 61.2 mmol, 1.2 eq) in THE (50 mL) was stirred at 0° C. for 0.5 hr under N₂ atmosphere. Then DIAD (12.4 g, 61.2 mmol, 11.9 mL, 1.2 eq) was added at 0° C. The mixture was stirred at 25° C. for 16 hr. On completion, the mixture was concentrated in vacuum to give a residue. The residue was purified by column chromatography (SiO₂, Petroleum ether/THF=1/0 to 4/1) and reversed-phase HPLC (0.1% FA condition 0%-50% ACN) to give tert-butyl (3R)-3-(2-methylpyrazol-3-yl)oxypiperidine-1-carboxylate (2.80 g, 9.35 mmol, 18% yield) as a colorless oil. ¹H NMR (400 MHz, CDCl₃-d) δ=7.30 (s, 1H), 5.52 (br s, 1H), 5.14-4.84 (m, 1H), 4.17-4.12 (m, 1H), 3.79-3.68 (m, 1H), 3.61 (s, 3H), 3.58-3.50 (m, 1H), 3.31 (d, J=17.2 Hz, 1H), 1.99-1.77 (m, 4H), 1.38 (s, 9H). LCMS: m/z 282.1 (M+1)

Step 2. To a solution of tert-butyl (3R)-3-(2-methylpyrazol-3-yl)oxypiperidine-1-carboxylate (2.0 g, 7.11 mmol, 1.0 eq) in ACN (20 mL) was added NBS (1.27 g, 7.11 mmol, 1.0 eq) at 0° C. The mixture was stirred at 0° C. for 2 hr. On completion, the mixture was quenched with water saturated Na₂SO₃ (100 mL), extracted with ethyl acetate (45 mL×3), the combined organic phase was washed with brine dried over anhydrous sodium sulfate, filtered and concentrated in vacuum to give a residue. The residue was purified by column chromatography (SiO₂, PE/EA=1/0 to 2/1) to give tert-butyl (3R)-3-(4-bromo-2-methyl-pyrazol-3-yl)oxypiperidine-1-carboxylate (1.20 g, 3.33 mmol, 47% yield) as a colorless oil. ¹H NMR (400 MHz, CDCl₃-d) δ=7.29 (s, 1H), 4.68 (br d, J=3.6 Hz, 1H), 3.76-3.72 (m, 1H), 3.65 (s, 3H), 3.59-3.54 (m, 1H), 3.41-3.23 (m, 2H), 1.93-1.83 (m, 4H), 1.42 (br s, 9H).

Step 3. A mixture of tert-butyl (3R)-3-(4-bromo-2-methyl-pyrazol-3-yl)oxypiperidine-1-carboxylate (1.05 g, 2.91 mmol, 1.0 eq), triisopropyl-[2-[1-tetrahydropyran-2-yl-5-(4,4,5,5-tetramethyl-1,3,2-dioxaborolan-2-yl) indazol-3-yl]ethynyl]silane (1.78 g, 3.50 mmol, 1.2 eq), Cs₂CO₃ (2.85 g, 8.74 mmol, 3.0 eq), ditert-butyl(cyclopentyl)phosphane;dichloropalladium;iron (190 mg, 0.291 mmol, 0.1 eq) in dioxane (10 mL) and H₂O (2 mL) was degassed and purged with N₂ for 3 times, and then the mixture was stirred at 80° C. for 4 hr under N2 atmosphere. On completion, the mixture was diluted with water (50 mL) and extracted with ethyl acetate (30 mL×3), the combined organic phase was washed with brine dried over anhydrous sodium sulfate, filtered and concentrated in vacuum to give a residue. The residue was purified by column chromatography (SiO₂, Petroleum ether/Ethyl acetate=1/0 to 1/1) to give tert-butyl (3R)-3-[2-methyl-4-[1-tetrahydropyran-2-yl-3-(2-triisopropylsilylethynyl) indazol-5-yl]pyrazol-3-yl]oxypiperidine-1-carboxylate (1.56 g, 2.10 mmol, 72% yield) as a brown oil. LCMS: m/z 662.4 (M+1)

Step 4. To a solution of tert-butyl (3R)-3-[2-methyl-4-[1-tetrahydropyran-2-yl-3-(2-triisopropylsilylethynyl) indazol-5-yl]pyrazol-3-yl]oxypiperidine-1-carboxylate (1.5 g, 2.27 mmol, 1.0 eq) in DCM (20 mL) was added ZnBr₂ (2.55 g, 11.3 mmol, 5.0 eq). The mixture was stirred at 25° C. for 16 hr. On completion, the mixture was diluted with water (100 mL) and extracted with ethyl acetate (50 mL×3), the combined organic phase was dried over anhydrous sodium sulfate, filtered and concentrated to give a residue. The residue was purified by column chromatography (SiO₂, DCM/MeOH=1/0 to 0/1) to give triisopropyl-[2-[5-[1-methyl-5-[[(3R)-3-piperidyl]oxy]pyrazol-4-yl]-1-tetrahydropyran-2-yl-indazol-3-yl]ethynyl]silane (600 mg, 0.918 mmol, 40% yield,) as a colorless gum. LCMS: m/z 562.5 (M+1)

Step 5. To a solution of triisopropyl-[2-[5-[1-methyl-5-[[(3R)-3-piperidyl]oxy]pyrazol-4-yl]-1-tetrahydropyran-2-yl-indazol-3-yl]ethynyl]silane (570 mg, 1.01 mmol, 1.0 eq) and 5-(bromomethyl)-4-iodo-1,3-dimethyl-pyrazole (383 mg, 1.22 mmol, 1.2 eq) in DMF (10 mL) was added K₂CO₃ (421 mg, 3.04 mmol, 3.0 eq). The mixture was stirred at 80° C. for 16 hr. On completion, the mixture was diluted with water (50 mL) and extracted with ethyl acetate (25 mL×3), the combined organic phase was washed with brine dried over anhydrous sodium sulfate, filtered and concentrated in vacuum to give 2-[5-[5-[[(3R)-1-[(4-iodo-2,5-dimethyl-pyrazol-3-yl)methyl]-3-piperidyl]oxy]-1-methyl-pyrazol-4-yl]-1-tetrahydropyran-2-yl-indazol-3-yl]ethynyl-triisopropyl-silane (800 mg, crude) as a colorless oil. LCMS: m/z 796.6 (M+1)

Steps 6-9 were conducted in a similar manner to those described in General Method H to give Ex. 122. [01472](2S)-1-[(10S,17E)-8,10,12,16-tetramethyl-2,8,10,11,12,13-hexahydro-14H-3,5-ethenotripyrazolo[3,4f:3′,4′-j:4″,3″-n][1,4]oxazacyclopentadecin-14-yl]propan-2-ol (Ex. 123) and (2S)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol (Ex. 124)

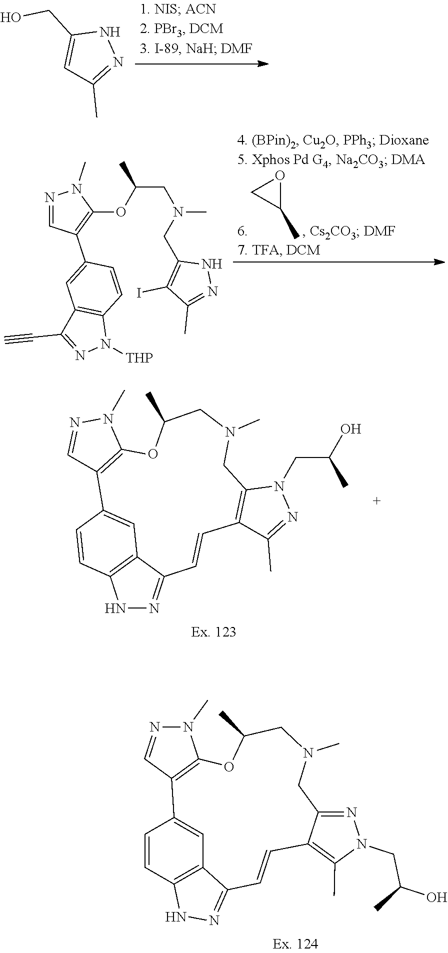

Steps 1-3 was conducted according to General Method O.

Steps 4-7 were conducted similarly to those described in Ex. 98 to give Ex. 123 and Ex. 124.

### Preparation of 2-[5-(bromomethyl)-4-iodo-3-methyl-pyrazol-1-yl]ethanol (I-125)

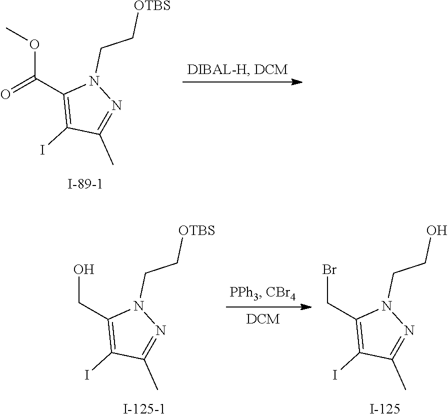 [SMILES: COC(=O)c1c(I)c(C)nn1CCO[Si](C)(C)C(C)(C)C ; Cc1nn(CCO[Si](C)(C)C(C)(C)C)c(CO)c1I ; Cc1nn(CCO)c(CBr)c1I]

Step 1. To a solution of methyl 2-[2-[tert-butyl(dimethyl)silyl]oxyethyl]-4-iodo-5-methyl-pyrazole-3-carboxylate (2.00 g, 4.71 mmol, 1 eq) in THF (40 mL) was added DIBAL-H (268 mg, 7.07 mmol, 1.5 eq) at 0° C. .The mixture was stirred at 0° C. for 12 hr. On completion, the mixture was quenched with water (20 mL) and extracted with ethyl acetate (25 mL×3), the combined organic phase was dried over anhydrous sodium sulfate, filtered and concentrated to give a residue. The residue was purified by column chromatography (SiO₂, Petroleum ether/Ethyl acetate=1:1 to 1:1) to give [2-[2-[tert-butyl(dimethyl)silyl]oxyethyl]-5-methyl-pyrazol-3-yl]methanol (700 mg, 2.59 mmol, 54% yield) as a yellow oil. LCMS: m/z 397.1 (M+1)

Step 2. To a solution of [2-[2-[tert-butyl(dimethyl)silyl]oxyethyl]-4-iodo-5-methyl-pyrazol-3-yl]methanol (2.5 g, 6.31 mmol, 1 eq) and PPh₃ (2.48 g, 9.46 mmol, 1.5 eq) in DCM (40 mL) was added dropwise CBr₄ (3.14 g, 9.46 mmol, 1.5 eq) at 0° C. The resulting mixture was stirred at 25° C. for 4 hr. On completion, the mixture was quenched with water (20 mL) and extracted with DCM (25 mL×3), the combined organic phase was dried over anhydrous sodium sulfate, filtered and concentrated to give a residue. The residue was purified by column chromatography (SiO₂, Petroleum ether/Ethyl acetate=0:1 to 0:1) to give 2-[5-(bromomethyl)-4-iodo-3-methyl-pyrazol-1-yl]ethanol (264 mg, 0.765 mmol, 12% yield) as a white solid. LCMS: m/z 344.7 (M+1)

### Preparation of 2-[(10S,17E)-19-chloro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 125)

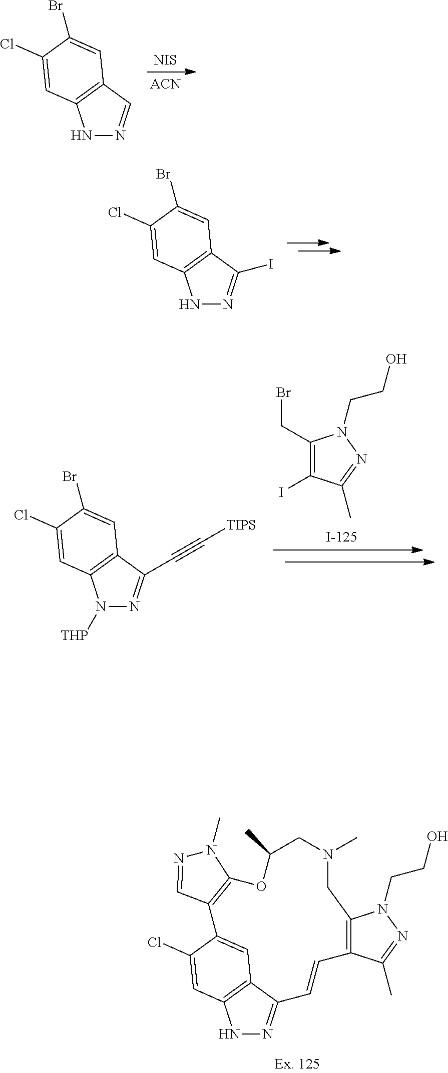

Step 1. To a solution of 5-bromo-6-chloro-1H-indazole (5.00 g, 21.6 mmol, 1 eq) in ACN (200 mL) was added NIS (7.29 g, 32.4 mmol, 1.5 eq). The mixture was stirred at 25° C. for 4 h. On completion, the mixture was quenched with water (20 mL) and extracted with ethyl acetate (25 mL×3), the combined organic phase was dried over anhydrous sodium sulfate, filtered and concentrated to give 5-bromo-6-chloro-3-iodo-1H-indazole (7.70 g, 99% yield) as a red solid. LCMS: m/z 356.8 (M+1)

Steps 2-3 were conducted in a manner similar to those described for C-1-1.

Steps 4-10 were conducted in a manner similar to those described in General Method M using I-125 in step 6 to afford Ex. 125.

### Preparation of (10S,17E)-8,10,12,14,15-pentamethyl-2,10,11,12,13,15-hexahydro-8H-3,5-ethenotripyrazolo[4,3-f:3′,4′-j:4″,3″-n][1,4]oxazacyclopentadecine (Ex. 126)

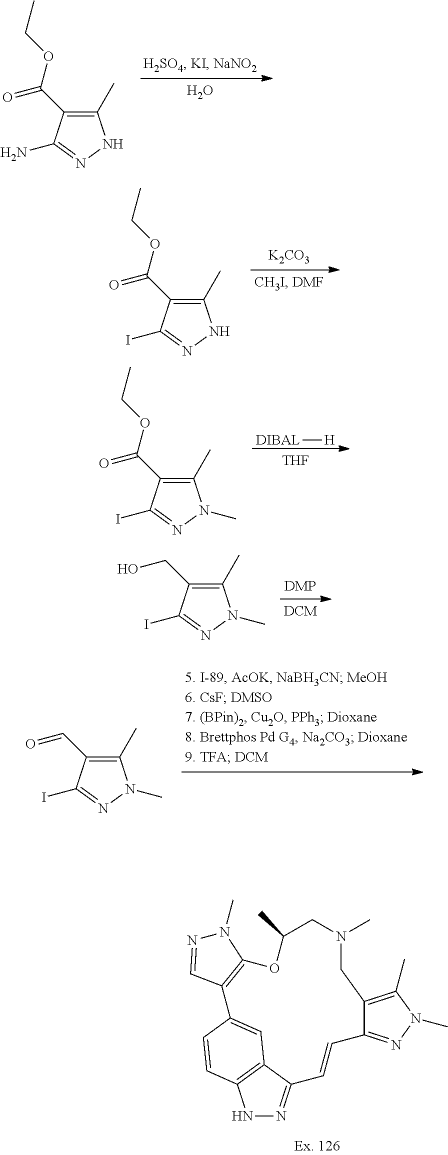

Step 1. Ethyl 3-amino-5-methyl-1H-pyrazole-4-carboxylate (5.00 g, 29.5 mmol, 1 eq) was added in portions to a solution of H₂SO₄ (15 mL) in H₂O (12 mL) at 0° C. The resulting solution was stirred at 0° C. for 30 min. A solution of NaNO₂ (3.06 g, 44.3 mmol, 1.5 eq) in H₂O (12 mL) was added dropwise to the reaction mixture at 0° C. The resulting solution was stirred at 0° C. for another hour. A solution of KI (10.5 g, 63.2 mmol, 2.14 eq) in H₂O (12.5 mL) was then added dropwise. The resulting solution was stirred at 25° C. for 8 h. On completion, the mixture was quenched with water (50 mL) and extracted with ethyl acetate (20 mL×3), the combined organic phase was dried over anhydrous sodium sulfate, filtered and concentrated to give a residue. The residue was purified by column chromatography (SiO₂, PE:THF=1:0 to 4:1) to give ethyl 3-iodo-5-methyl-1H-pyrazole-4-carboxylate (3.00 g, 36% yield) as an orange solid. LCMS: m/z 280.9 (M+1)

Step 2. To a solution of ethyl 3-iodo-5-methyl-1H-pyrazole-4-carboxylate (1 g, 3.57 mmol, 1 eq) in DMF (10 mL) was added K₂CO₃ (1.48 g, 10.7 mmol, 3 eq) and CH3I (658 mg, 4.64 mmol, 1.3 eq). The mixture was stirred at 25° C. for 2 h. On completion, the mixture was quenched with water (20 mL) and extracted with ethyl acetate (10 mL×3), the combined organic phase was dried over anhydrous sodium sulfate, filtered and concentrated to give a residue. The residue was purified by column chromatography (SiO₂, PE:THF=1:0 to 100:13) to give ethyl 3-iodo-1,5-dimethyl-pyrazole-4- carboxylate (530 mg, 50% yield) as yellow solid. ¹H NMR (400 MHz, DMSO-d₆) δ=4.25-4.18 (m, 2H), 3.76 (s, 3H), 2.48 (s, 3H), 1.29 (t, J=7.2 Hz, 3H). LCMS: m/z 294.9 (M+1)

Step 3. To a solution of ethyl 3-iodo-1,5-dimethyl-pyrazole-4-carboxylate (500 mg, 1.70 mmol, 1 eq) in THE (6 mL) was degassed and purged with N₂ for 3 times, and then DIBAL-H (1 M, 4 mL, 2.5 eq) was added dropwise at 0° C. The mixture was stirred at 25° C. for 1 h under N2 atmosphere. On completion, the mixture was quenched with MeOH (30 mL), filtered and concentrated to give a residue. The residue was purified by column chromatography (SiO₂, Dichloromethane:Methanol=1:0 to 10:1) to give (3-iodo-1,5-dimethyl-pyrazol-4-yl)methanol (380 mg, 88% yield) as white solid. LCMS: m/z 252.9 (M+1)

Step 4. To a solution of (3-iodo-1,5-dimethyl-pyrazol-4-yl)methanol (320 mg, 1.27 mmol, 1 eq) in DCM (5 mL) was added DMP (646 mg, 1.52 mmol, 1.2 eq). The mixture was stirred at 25° C. for 2 h. On completion, the mixture was quenched with aq. Na₂SO₃ (10 mL) and extracted with DCM (8 mL×5), the combined organic phase was dried over anhydrous sodium sulfate, filtered and concentrated to give 3-iodo-1,5-dimethyl-pyrazole-4-carbaldehyde (250 mg, 78% yield) as white solid. LCMS: m/z 250.9 (M+1)

Steps 5. The mixture of (2S)—N-methyl-2-[2-methyl-4-[1-tetrahydropyran-2-yl-3-(2- triisopropylsilylethynyl) indazol-5-yl]pyrazol-3-yl]oxy-propan-1-amine (458 mg, 0.833 mmol, 1 eq) and 3-iodo-1,5-dimethyl-pyrazole-4-carbaldehyde (250 mg, 0.999 mmol, 1.2 eq) in MeOH (3 mL) was added AcOK (817 mg, 8.33 mmol, 10 eq). The mixture was stirred at 25° C. for 0.2 h. Then NaBH₃CN (157 mg, 2.50 mmol, 3 eq) was added to the mixture, the mixture was stirred at 25° C. for 12 h. On completion, the mixture was quenched with water (1 mL) and concentrated to give a residue. The residue was purified by column chromatography (SiO₂, PE:TFH=1:0 to 2:1) to give (2S)—N-|(3-iodo-1,5-dimethyl-pyrazol-4-yl)methyl]-N-methyl-2-[2-methyl-4-[1-tetrahydropyran-2-yl-3-(2-triisopropylsilylethynyl) indazol-5-yl]pyrazol-3-yl]oxy-propan-1-amine (402 mg, 61% yield) as white solid. LCMS: m/z 784.3 (M+1)

Steps 6-7 were conducted in a manner similar to those described in General Method N

Step 8. To a solution of (2S)—N-[(3-iodo-1,5-dimethyl-pyrazol-4-yl)methyl]-N-methyl-2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxy-propan-1-amine (158 mg, 0.209 mmol, 1 eq) in dioxane (8 mL) was added Na₂CO₃ (1.5 M, 418 μL, 3 eq) and BrettPhos (Pd, G4) (19.2 mg, 0.021 mmol, 0.1 eq). The mixture was degassed and purged with N2 for 3 times, and then the mixture was stirred at 80° C. for 2 h under N2 atmosphere. On completion, the mixture was dried over anhydrous sodium sulfate, filtered and concentrated to give a residue. The residue was purified by column chromatography (SiO₂, PE:THF=1:0 to 1:2) to give (8S,17E)-5,8,10,13,14-pentamethyl-21-tetrahydropyran-2-yl-7-oxa-4,5,10,14,15,20,21-heptazapentacyclo[17.5.2.0^{2,6}.0^{12,16}0.0^{22,26}]hexacosa-1(25),2(6),3,12,15,17,19,22(26),23-nonaene (61.0 mg, 58% yield) as white oil. LCMS: m/z 502.2 (M+1)

Step 9 was conducted in a manner similar to General Method N to afford Ex. 126.

Preparation of Ex. 127 and Ex. 128 were prepared in a manner similar to those described for Ex. 123 and 124 using (2R)-2-methyloxirane.

### Preparation of 2-[(10S,17E)-16-ethyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol. (Ex. 129)

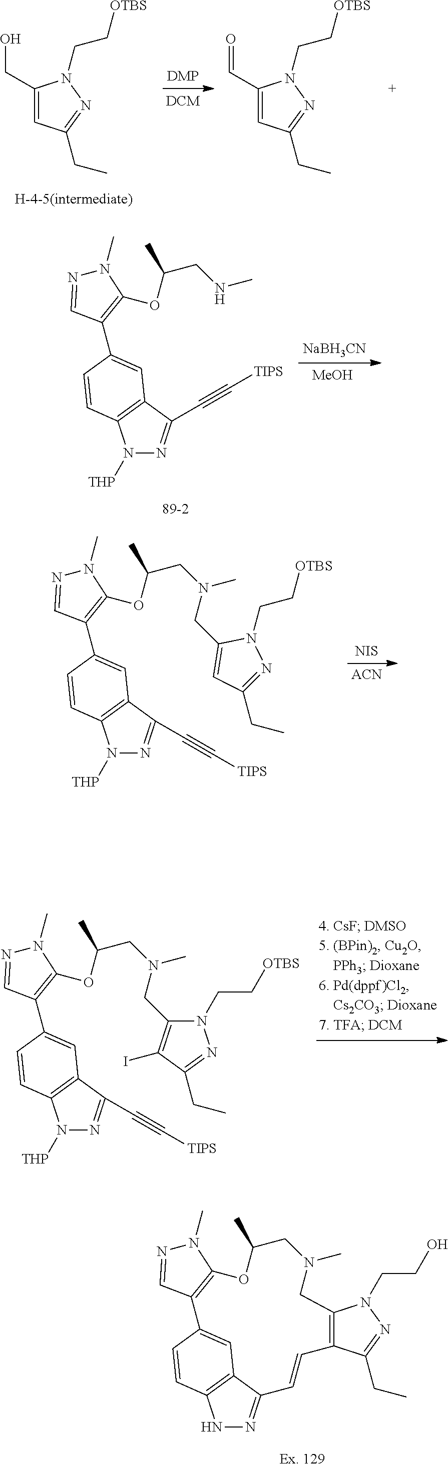

Step 1. To a mixture of [2-[2-[tert-butyl(dimethyl)silyl]oxyethyl]-5-ethyl-pyrazol-3-yl]methanol (3.50 g, 12.3 mmol, 1 eq) in DCM (40 mL) was added DMP (7.83 g, 18.4 mmol, 1.5 eq) at 0° C., the reaction mixture was stirred at 25° C. for 2 hours. On completion, the reaction mixture were quenched with sat·Na₂S₂O₃ (15 mL) and sat·NaHCO₃ (15 mL) under stirring, then the mixture was diluted with water (50 mL) and extracted with EA (2×50 mL). The combined organic layers was dried over Na₂SO₄, filtered and concentrated in vacuo to afford compound 2-[2-[tert-butyl(dimethyl)silyl]oxyethyl]-5-ethyl-pyrazole-3-carbaldehyde (3.05 g, 87% yield) as yellow oil. ¹H NMR (400 MHz, DMSO-d₆) δ 9.85 (s, 1H), 6.82 (s, 1H), 4.53 (t, J=5.2 Hz, 2H), 3.86 (t, J=5.2 Hz, 2H), 2.59 (q, J=7.6 Hz, 2H), 1.17 (t, J=7.6 Hz, 3H), 0.74 (s, 9H), -0.12-0.18 (m, 6H).

Step 2. To a mixture of (2S)—N-methyl-2-[2-methyl-4-[1-tetrahydropyran -2-yl-3- (2- triisopropylsilylethynyl) indazol-5-yl]pyrazol-3-yl]oxy-propan-1-amine (5.34 g, 9.72 mmol, 0.9 eq) in MeOH (40 mL) was added 2-[2-[tertbutyl (dimethyl) silyl]oxyethyl]-5-ethyl -pyrazole-3-carbaldehyde (3.05 g, 10.8 mmol, 1 eq) and NaBH₃CN (1.02 g, 16.2 mmol, 1.5 eq), the reaction mixture was stirred at 25° C. for 12 hours. On completion, the mixture was diluted with water (50 mL) and extracted with EA (2×50 mL). The combined organic layers was dried over Na₂SO₄, filtered and concentrated in vacuo to afford compound t(2S)—N-[[2-[2-[tert-butyl(dimethyl)silyl]oxyethyl]-5-ethyl- pyrazol-3-yl]methyl]-N-methyl-2-[2-methyl-4-[1-tetrahydropyran-2-yl-3-(2-triisopropylsilylethynyl) indazol-5-yl]pyrazol-3-yl]oxy-propan-1-amine (7.50 g, 85% yield) as colorless oil. ¹H NMR (400 MHz, DMSO-d₆) δ 7.80 (d, J=8.8 Hz, 1H), 7.75 (s, 1H), 7.66-7.65 (m, 1H), 7.64-7.61 (m, 1H), 5.90-5.86 (m, 1H), 5.76 (d, J=1.6 Hz, 1H), 4.17-4.10 (m, 2H), 4.10-4.05 (m, 1H), 4.03 (d, J=7.2 Hz, 1H), 4.00-3.95 (m, 2H), 3.94-3.87 (m, 2H), 3.86-3.83 (m, 1H), 3.77-3.75 (m, 2H), 3.66 (s, 3H), 3.41 (d, J=4.8 Hz, 2H), 2.65 (dd, J=5.6, 12.8 Hz, 1H), 2.60-2.54 (m, 1H), 2.47-2.45 (m, 1H), 2.43 (d, J=7.6 Hz, 2H), 2.39-2.33 (m, 1H), 2.04 (d, J=14.4 Hz, 2H), 1.99 (s, 3H), 1.95 (s, 1H), 1.59 (s, 2H), 1.19-1.18 (m, 2H), 1.17 (d, J=1.6 Hz, 2H), 1.15 (s, 18H), 1.11-1.08 (m, 6H), 0.69 (s, 9H).

Step 3. To a mixture of (2S)—N-[[2-[2-[tert-butyl(dimethyl) silyl]oxyethyl]-5-ethyl-pyrazol-3-yl]methyl]-N-methyl-2-[2-methyl-4-[1-tetrahydropyran-2-yl-3-(2-triisopropylsilylethynyl) indazol-5-yl]pyrazol-3-yl]oxy-propan-1-amine (1.08 g, 1.32 mmol, 1 eq) in ACN (15 mL) was added NIS (446 mg, 1.98 mmol, 1.5 eq) at 0° C., the reaction mixture was stirred at 25° C. for 8 hours. On completion, the reaction mixture was quenched with sat·Na₂SO₃ solution (5 mL) under stirring, and then the residue was diluted with water (20 mL) and extracted with EA (2×20 mL). The combined organic layers was dried over Na₂SO₄, filtered and concentrated in vacuo to afford compound (2S)—N-[[2-[2-[tert-butyl(dimethyl)silyl]oxyethyl]-5-ethyl-4-iodo-pyrazol-3-yl]methyl]-N-methyl-2-[2-methyl-4-[1-tetrahydropyran-2-yl-3-(2-triisopropylsilylethynyl) indazol-5-yl]pyrazol-3-yl]oxy-propan-1-amine (1.40 g, crude) as yellow oil. LCMS: m/z 942.4 (M+1)

Steps 4-7 were conducted in a manner similar to those described in General Method H to give Ex. 129.

Ex. 130 was prepared in a manner similar to those described in Ex. 126 starting in step 2 with 3-iodo-5-methyl-1H-pyrazole-4-carboxylate.

### Preparation of (10S,17E)-8,10,12,14,16-pentamethyl-2,10,11,12,13,16-hexahydro-8H-3,5-ethenotripyrazolo[4,3-f:3′,4′-j:4″,3″-n][1,4]oxazacyclopentadecine (Ex. 131)

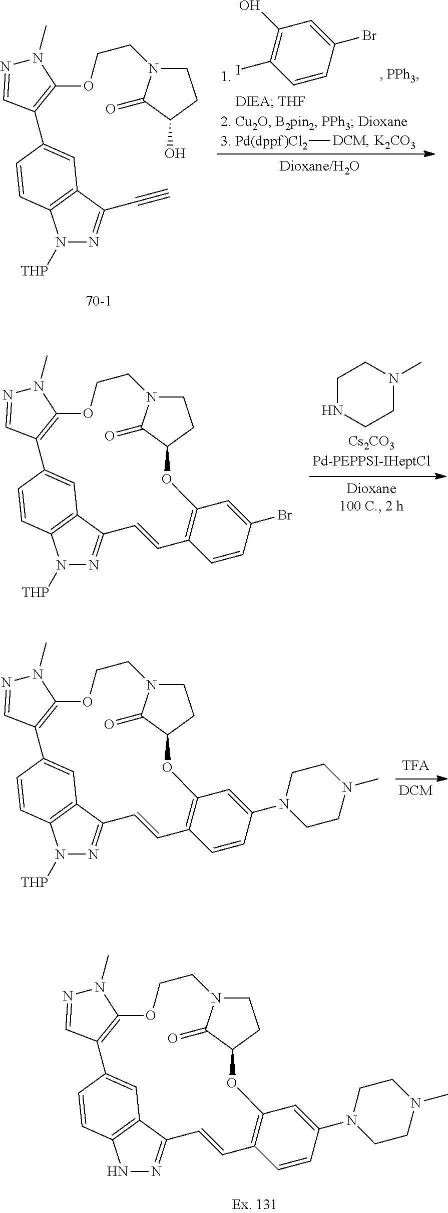

Steps 1-2 are described in General Method L.

Step 3. A mixture of (3R)-3-(5-bromo-2-iodo-phenoxy)-1-[2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxyethyl]pyrrolidin-2-one (200 mg, 0.233 mmol, 1.00 eq), K₂CO₃ (64.4 mg, 0.466 mmol, 2.00 eq), Pd(dppf)Cl₂·CH₂Cl₂ (19.0 mg, 0.023 mmol, 0.10 eq) in dioxane (10 mL) and H₂O (2 mL) was degassed and purged with N₂ for 3 times. The mixture was stirred at 90° C. for 2 h under N₂ atmosphere. On completion, the reaction mixture was concentrated under reduced pressure to give a residue. The residue was purified by column chromatography (SiO₂, Dichloromethane/Methanol=10/1 to 5/1) to give (13R,21E)-17-bromo-5-methyl-25-tetrahydropyran-2-yl-7,14-dioxa-4,5,10,24,25-pentazahexacyclo[21.5.2.1^{10,13}.0^{2,6}.0,^{15,20}.0^{26,30}]hentriaconta-1(29),2(6),3,15(20),16,18,21,23,26(30),27-decaen-31-one (54.0 mg, 0.089 mmol, 38% yield) as brown oil. ¹H NMR (400 MHz, DMSO-d₆) δ=8.01 (s, 1H), 7.81-7.74 (m, 2H), 7.72-7.66 (m, 1H), 7.65-7.60 (m, 1H), 7.49 (d, J=1.6 Hz, 1H), 7.25 (d, J=1.6 Hz, 1H), 5.91-5.84 (m, 1H), 5.16 (s, 1H), 4.39-4.28 (m. 1H), 4.12-4.05 (m, 1H), 3.89 (s, 6H), 3.84-3.84 (m, 1H), 3.80-3.76 (m, 3H), 3.60-3.41 (m, 2H), 2.47-2.36 (m, 2H), 2.19-2.02 (m, 2H), 1.83-1.69 (m, 1H), 1.60 (s, 2H). LCMS: m/z 606.1 (M+1)

Step 4. A mixture of (13R,21E)-17-bromo-5-methyl-25-tetrahydropyran-2-yl-7,14-dioxa-4,5,10,24,25-pentazahexacyclo[21.5.2.1^{10,13}.0^{2,6}.0^{15,20}.0^{26,30}]hentriaconta-1(29),2(6),3,15(20),16,18,21,23,26(30),27-decaen-31-one (44.0 mg, 0.073 mmol, 1.00 eq), 1-methylpiperazine (8.75 mg, 0.087 mmol, 1.20 eq), Cs₂CO₃ (71.2 mg, 0.218 mmol, 3.00 eq), Pd-PEPPSI-IHeptCl (7.08 mg, 0.0073 mmol, 0.10 eq) in dioxane (2 mL) was degassed and purged with N₂ for 3 times. The mixture was stirred at 100° C. for 2 h under N₂ atmosphere.

On completion, the reaction mixture was concentrated under reduced pressure to give a residue. The residue was purified by column chromatography (SiO₂, Dichloromethane/Methanol=1/0 to 10/1) to give (13R,21E)-5-methyl-17-(4-methylpiperazin-1-yl)-25-tetrahydropyran-2-yl-7,14-dioxa-4,5,10,24,25-pentazahexacyclo[21.5.2.1^{10,13}.0^{2,6}.0^{15,20}.0^{26,30}]hentriaconta-1(29),2(6),3,15(20),16,18, 21,23,26(30),27-decaen-31-one (44.0 mg, 0.071 mmol, 97% yield) as brown solid. LCMS: m/z 624.4 (M+1)

Step 5 was conducted in a manner described in General Method L to give Ex. 131.

Ex. 132 was prepared in a manner similar to those described in Ex. 131 starting with the appropriate phenol.

### Preparation of v2-[(10S,17E)-12-ethyl-8,10,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 133)

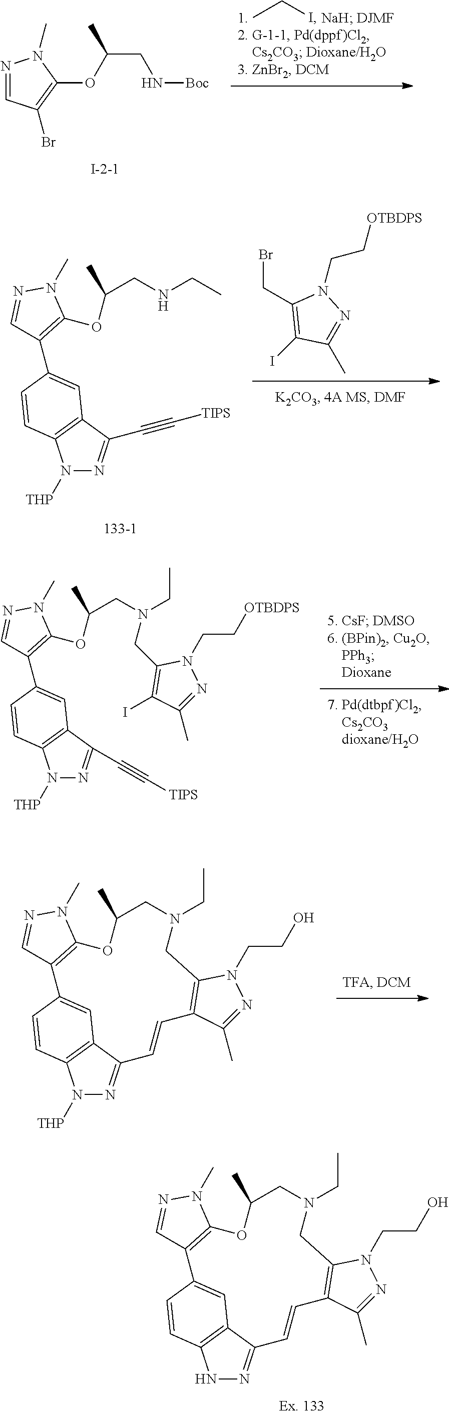

Example 133 was prepared following procedures similar to those described in General Method M with variations in steps 4 and 7 as detailed.

### Steps 1-3, General Method M

Step 4. To a mixture of (2S)—N-ethyl-2-[|2-methyl-4- |1-tetrahydropyran-2-yl-3- (2- triisopropylsilylethynyl) indazol-5-yl]pyrazol-3-yl]oxy-propan-1-amine (390 mg, 0.691 mmol, 1 eq) and 2-[5-(bromomethyl)-4-iodo-3-methyl-pyrazol-1-yl]ethoxy-tert-butyl-diphenyl-silane (1.21 g, 2.08 mmol, 3 eq), made similarly to I-114, in DMF (5 mL) was added K₂CO₃ (286 mg, 2.08 mmol, 3 eq) and 4A MS (1.00 g, 0.691 mmol, 1 eq), the reaction mixture was stirred at 50° C. for 12 hours. On completion, the residue was diluted with water (10 mL) and extracted with EA (2×10 mL). The combined organic layers was dried over Na₂SO₄, filtered and concentrated in vacuo. The residue was purified by column chromatography (SiO₂, 80/20% DCM:DCM/MeOH, DCM/MeOH=10:1) to afford compound ((2S)—N—[[2-[2-[tert-butyl(diphenyl)silyl]oxyethyl]-4-iodo-5-methyl-pyrazol-3-yl]methyl]-N-ethyl-2-[2-methyl-4-[1-tetrahydropyran -2- yl -3- (2- triisopropylsilylethynyl) indazol-5-yl]pyrazol-3-yl]oxy-propan -1- amine (717 mg, 0.672 mmol, 97% yield) as yellow oil. 1H NMR (400 MHz, DMSO-d6) δ 7.95 (s, 1H), 7.81 (d, J=8.8 Hz, 1H), 7.74-7.72 (m, 1H), 7.65 (s, 1H), 7.64-7.61 (m, 1H), 7.44-7.39 (m, 4H), 7.33 (s, 6H), 5.90-5.85 (m, 1H), 4.22-4.17 (m, 2H), 3.92 (s, 2H), 3.87 (s, 1H), 3.78 (t, J=4.8 Hz, 3H), 3.60 (s, 3H), 3.57-3.48 (m, 2H), 2.64 (dd, J=4.8, 13.6 Hz, 2H), 2.43 (d, J=7.6 Hz, 1H), 2.35-2.28 (m, 3H), 2.09 (s, 3H), 2.01-1.92 (m, 2H), 1.82-1.66 (m, 2H), 1.58 (s, 2H), 1.26-1.22 (m, 2H), 1.16-1.11 (m, 20H), 1.07 (s, 10H)

Steps 5-6 conducted according to General Method M.

Step 7. To a mixture of 2-[5-[[ethyl-[(2S)-2-[2-methyl-4-[1-tetrahydropyran-2-yl-3-[(E)-2-(4,4,5,5-tetramethyl-1,3,2-dioxaborolan-2-yl)vinyl]indazol-5-yl]pyrazol-3-yl]oxypropyl]amino]methyl]-4-iodo-3-methyl-pyrazol-1-yl]ethanol (70.0 mg, 0.088 mmol, 1 eq) in dioxane (1 mL) and H₂O (0.2 mL) was added Cs₂CO₃ (85.5 mg, 0.262 mmol, 3 eq) and ditert-butyl(cyclopentyl) phosphane;dichloropalladium;iron (5.71 mg, 0.009 mmol, 0.1 eq). The reaction mixture was stirred at 90° C. for 1 hour. On completion, the residue was diluted with water (20 mL) and extracted with EA (2×20 mL). The combined organic layers was dried over Na₂SO₄, filtered and concentrated in vacuo to give a residue. The residue was purified by column chromatography (SiO₂, 80/20% DCM:DCM/MeOH, DCM/MeOH=10:1) to afford 2-[(8S,17E)-10-ethyl-5,8,15-trimethyl-21-tetrahydropyran-2-yl-7-oxa-4,5,10,13,14,20,21-heptazapentacyclo[17.5.2.02,6.012,16.022,26]hexacosa-1(25),2(6),3,12(16),14,17,19,22(26),23-nonaen-13-yl]ethanol (30.0 mg, 0.055 mmol, 62% yield) as yellow oil. LCMS: m/z 546.4 (M+1)

Step 8. Ex. 133 was prepared following procedure described in General Method M.

### General Method P. Preparation of (10S,17E)-8,10,12,16-tetramethyl-2,8,10,11,12,13-hexahydro-3,5-etheno[1,2]oxazolo[3,4-f]dipyrazolo[3,4-j:4′,3′-n][1,4]oxazacyclopentadecine (Ex. 134)

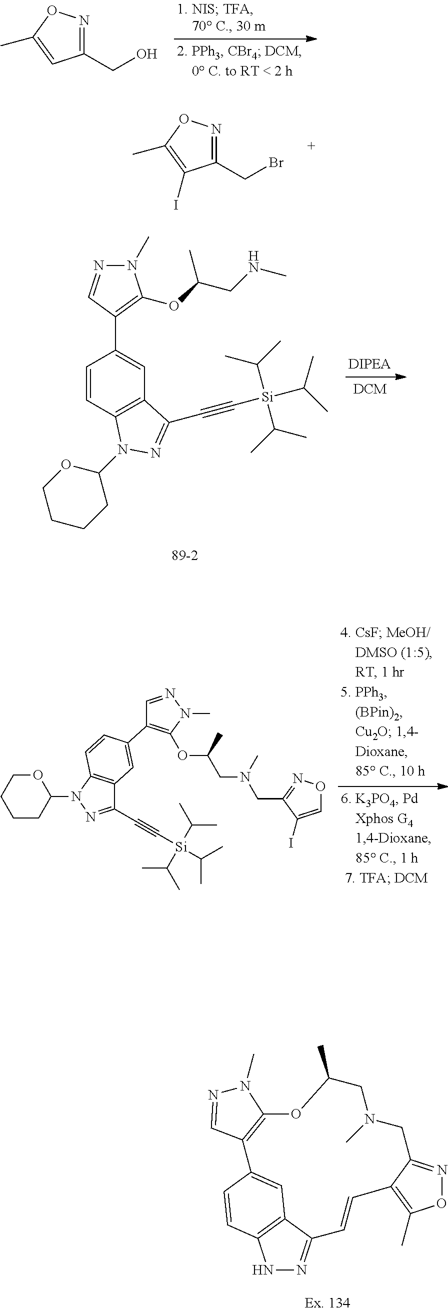

Steps 1-2 were conducted according to procedures described in General Method 0.

Step 3. Step 3. To (2S)—N-methyl-2-[2-methyl-4-[l-tetrahydropyran-2-yl-3-(2-triisopropylsilylethynyl) indazol-5-yl]pyrazol-3-yl]oxy-propan-1-amine, 100 mg, 0.182 mmol) in anhydrous DCM (1 mL) was added DIPEA (0.546 mmol, 95 μL) followed by 3-(bromomethyl)-4-iodo-5-methyl-isoxazole (66 mg, 0.218 mmol). Stirred at RT for 18 hr. Flash column chromatography (automated system, 12 g silica, 0-60% EA in Hexanes) provided (2S)—N-[(4-iodo-5-methyl-isoxazol-3-yl)methyl]-N-methyl-2-[2-methyl-4-[1-tetrahydropyran-2-yl-3-(2-triisopropylsilylethynyl) indazol-5-yl]pyrazol-3-yl]oxy-propan-1-amine (73.2 mg, 0.095 mmol, 52% yield). LCMS: m/z 771.1 (M+1)

Steps 4-7 were conducted in a manner similar to those described in General Method M to give Ex. 134.

### Preparation of I-135

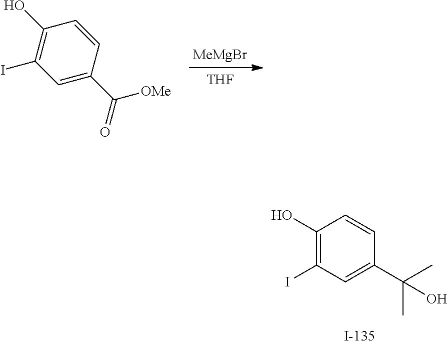

I-135 was prepared in a manner similar to those described for Ex. 105

### Preparation of (10R,16E)-14-(2-hydroxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one (Ex. 135)

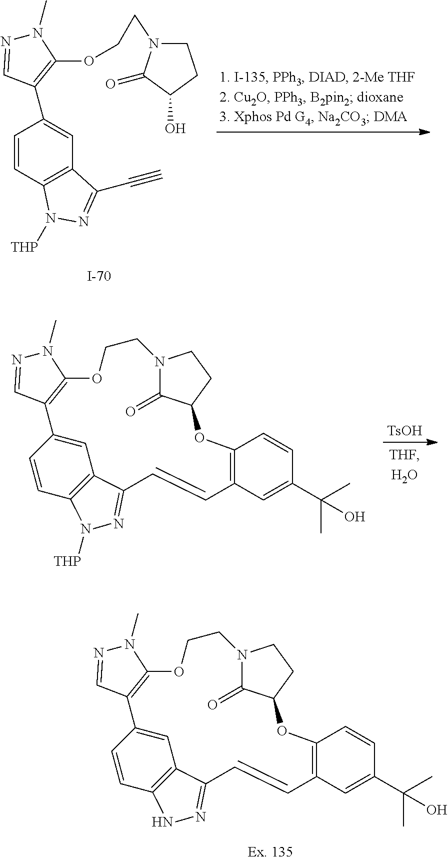

Steps 1-3 were conducted in a similar manner to those described in General Method L.

Step 4. The mixture of (13R,21E)-18-(1-hydroxy-1-methyl-ethyl)-5-methyl-25-tetrahydropyran-2-yl-7,14-dioxa-4,5,10,24,25-pentazahexacyclo[21.5.2.110,13.02,6. 015,20.026,30]hentriaconta-1(29),2(6),3,15(20),16,18,21,23,26(30),27-decaen-31-one (45.0 mg, 0.077 mmol, 1 eq) and TsOH (19.9 mg, 0.115 mmol, 1.5 eq) in H₂O (0.4 mL) and THF (2 mL) was stirred at 60° C. for 1 h. On completion, the mixture was diluted with DCM (10 mL) and adjusted to pH=8 with sat. NaHCO₃ (20 ml). The mixture was extracted with DCM (10 mL*3), and washed with brine (20 mL). The organic layer was dried over Na₂SO₄, filtered and concentrated to give the residue. The residue was then purified by prep-HPLC (column: Phenomenex luna C18 150*25 mm*10 um; mobile phase: [water(FA)-ACN]; B %: 19%-49%,8 min) to give Ex. 135 (7.63 mg, 0.137 mmol, 18% yield, 98.3% purity, FA) as an off-white solid.

### Preparation of tert-butyl 4-[5-(bromomethyl)-4-iodo-1-methyl-pyrazol-3-yl]oxypiperidine-1-carboxylate (I-136)

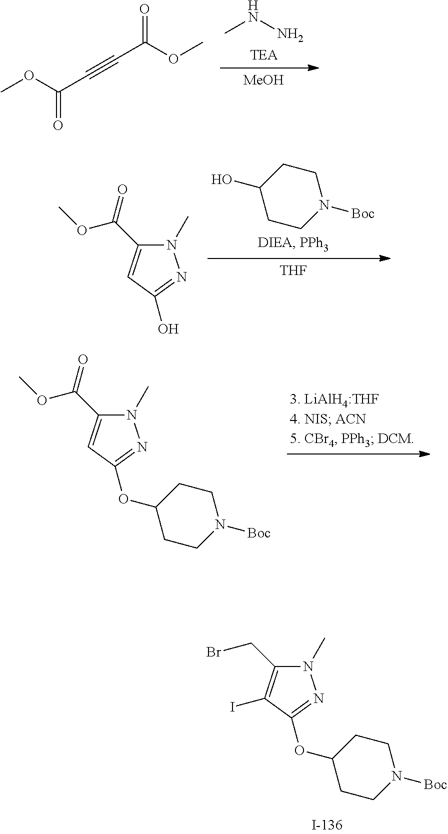

Step 1. To a solution of dimethyl but-2-ynedioate (50.0 g, 352 mmol, 1.00 eq) in MeOH (500 mL) was added TEA (71.2 g, 704 mmol, 97.9 mL, 2.00 eq) and Methylhydrazine;sulfuric acid (50.7 g, 352 mmol. 1.00 eq) at 0° C. The mixture was stirred at 25° C. for 16 h. On completion, the mixture was concentrated in reduced pressure. The residue was purified by column chromatography (SiO₂, Petroleum ether/Ethyl acetate=1/0 to 0/1) to give methyl 5-hydroxy-2-methyl-pyrazole-3-carboxylate (24.0 g, 154 mmol, 44% yield) as a yellow solid. ¹H NMR (400 MHz, DMSO-d₆) δ=10.03 (s, 1H), 3.87 (s, 3H), 3.78 (s, 3H).

Step 2. To a solution of methyl 5-hydroxy-2-methyl-pyrazole-3-carboxylate (2.00 g, 12.8 mmol, 1.00 eq) in THF (15 mL) was added tert-butyl 4-hydroxypiperidine-1-carboxylate (5.16 g, 25.6 mmol, 2.00 eq), PPh₃ (6.72 g, 25.6 mmol, 2.00 eq).The mixture was stirred at 25° C. for 1 h. DIAD (5.18 g, 25.6 mmol, 4.98 mL, 2.00 eq) was added into the mixture at 0° C. The mixture was stirred at 25° C. for 15 h under N2. On completion, the mixture was concentrated in reduced pressure. The crude product was triturated with petroleum ether at 25° C. for 10 min. The filtrate was concentrated. The residue was purified by column chromatography (SiO₂, Petroleum ether/Ethyl acetate=1/0 to 2/1) to give tert-butyl 4-(5-methoxycarbonyl-1-methyl-pyrazol-3-yl) oxypiperidine-1-carboxylate (4.35 g, 12.8 mmol, 100% yield) as a yellow oil. LCMS: m/z 284.1 (M+1)

Steps 3-5 were performed in a manner similar to those described in the synthesis of 119-2 to give I-136.

### Preparation of (10S,17E)-8,10,12,14-tetramethyl-16-[(piperidin-4-yl)oxy]-2,10,11,12,13,14-hexahydro-8H-3,5-ethenotripyrazolo[3,4-f:3′,4′-j:4″,3″-n][1,4]oxazacyclopentadecine (Ex. 136)

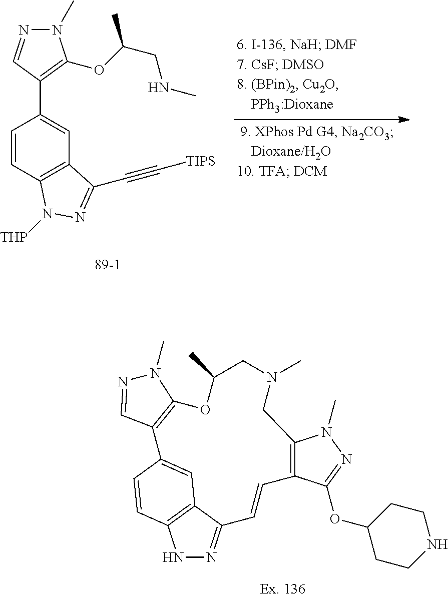

Steps 6-8 were conducted in manner similar to those described in General Method H

Step 9-10 was conducted in a manner similar to those described in General Method L to afford Ex. 136.

Ex. 137 was prepared in a similarly to Ex. 122 using 119-2 in step 5.

General Method Q. Preparation of 2-[(10S,17E)-6,8,10,12,16-pentamethyl-2,8,10,11,12,13-hexahydro-14H-3,5-ethenotripyrazolo[3,4-f:3′,4′-j:4″,3″-n][1,4]oxazacyclopentadecin-14-yl]ethan-1-o (Ex. 138)

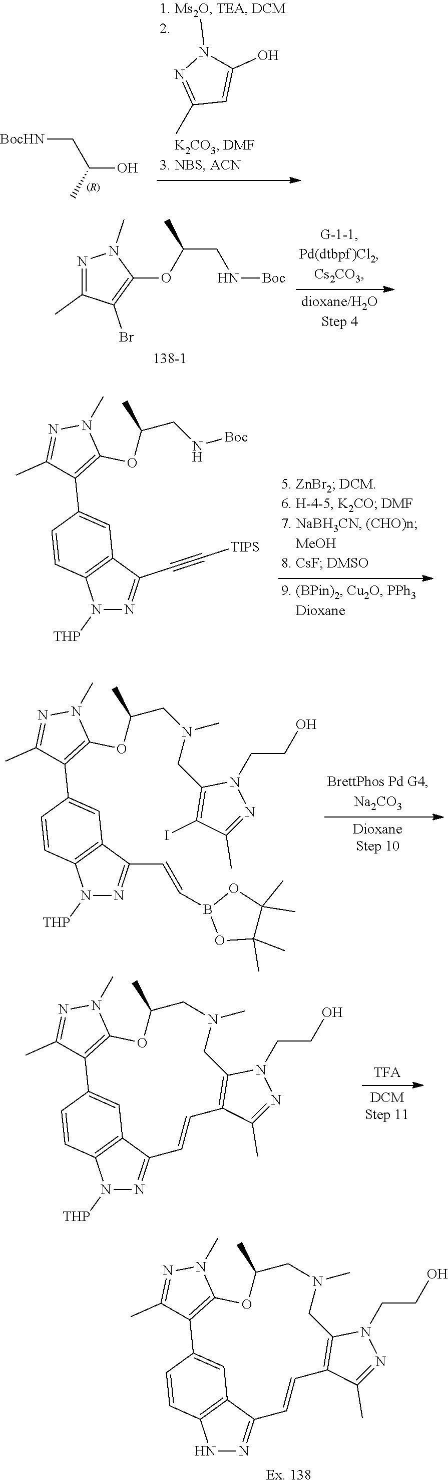

Steps 1-3 were conducted in a manner similar to those described in General Method M.

Step 4 was conducted in a manner similar to that described in General Method I.

Steps 5 were conducted in a manner similar to those described in General Method M.

Step 6. To a solution of (2S)-2-[2,5-dimethyl-4-[1-tetrahydropyran-2-yl-3-(2-triisopropylsilyle thynyl) indazol-5-yl]pyrazol-3-yl]oxypropan-1-amine (2.60 g, 4.73 mmol, 1 eq) and 2-[5-(bromomethyl)-4-iodo-3-methyl-pyrazol-1-yl]ethoxy-tert-butyl-diphenyl-silane (3.31 g, 5.67 mmol, 1.2 eq) in DMF (60 mL) was added K₂CO₃ (1.96 g, 14.1 mmol, 3 eq).

The mixture was stirred at 25° C. for 2 h. On completion, the mixture was quenched with water (180 mL) and extracted with EA (125 mL×3), the combined organic phase was dried over anhydrous sodium sulfate, filtered and concentrated to give (2S)—N-[[2-[2-[tert-butyl(diphenyl)silyl]oxyethyl]-4-iodo-5-methyl-pyrazol-3-yl]methyl]-2-[2,5-dimethyl-4-[1-tetrahydropyran-2-yl-3-(2-triisopropylsilylethynyl) indazol-5-yl]pyra zol-3-yl]oxy-propan-1-amine (3.20 g, 3.04 mmol, 64% yield) as a yellow oil. LCMS: m/z 1052.6 (M+1)

Step 7. To a solution of (2S)—N-[[2-[2-[tert-butyl(diphenyl)silyl]oxyethyl]-4-iodo-5-methyl-pyrazol-3-yl]methyl]-2-[2,5-dimethyl-4-[1-tetrahydropyran-2-yl-3-(2-triisopropylsilyl ethynyl) indazol-5-yl]pyrazol-3-yl]oxy-propan-1-amine (3.20 g, 3.04 mmol, 1 eq) and (CHO)ₙ (621 mg, 18.2 mmol, 6 eq) in MeOH (50 mL) was added NaBH₃CN (382 mg, 6.08 mmol, 2.0 eq) at 0° C. The mixture was stirred at 0° C. for 2 h. On completion, the mixture was filtered and concentrated to give (2S)—N-[[2-[2-[tert-butyl(diphenyl)silyl]oxyethyl]-4-iodo-5-methyl-pyrazol-3-yl]methyl]-2-[2,5-dimethyl -4-[1-tetrahydropyran-2-yl-3-(2-triisopropylsilylethynyl) indazol-5-yl]pyrazol-3-yl]oxy-N-methyl-propan-1-amine (3.20 g, 3.00 mmol, 98% yield) as a white solid. LCMS: m/z 1066.3(M+1)

Remaining steps 8-11 were conducted according to Ex. 126 to give Ex. 138.

### Preparation of (2S)-2-(methylsulfonylmethyl)azetidine, (I-139)

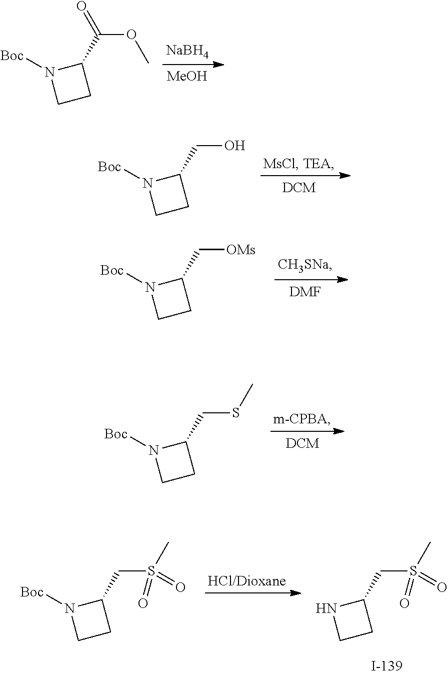

Step 1. To a solution of O1-tert-butyl 02-methyl (2S)-azetidine-1,2-dicarboxylate (5.00 g, 23.2 mmol, 1 eq) in MeOH (80 mL) was added NaBH₄ (4.39 g, 116 mmol, 5 eq). The mixture was stirred at 0° C. for 2 h. On completion, The mixture was quenched with water (120 mL) and extracted with ethyl acetate (125 mL×3), the combined organic phase was dried over anhydrous sodium sulfate, filtered and concentrated to give tert-butyl (2S)-2-(hydroxymethyl)azetidine-1-carboxylate (5.00 g, crude) as a colourless oil

Step 2. To a solution of tert-butyl (2S)-2-(hydroxymethyl)azetidine-1-carboxylate (4.30 g, 22.9 mmol, 1 eq) in DCM (100 mL) was added TEA (11.6 g, 114 mmol, 5 eq) and MsCl (15.7 g, 137 mmol, 6 eq). The mixture was stirred at 0° C. for 2 h. On completion, the mixture was quenched with water (100 mL) and extracted with DCM (120 mL×3), the combined organic phase was dried over anhydrous sodium sulfate, filtered and concentrated to give tert-butyl (2S)-2-(methylsulfonyloxymethyl) azetidine-1-carboxylate (8.00 g, crude) as a yellow oil.

Step 3. To a solution of tert-butyl (2S)-2-(methylsulfonyloxymethyl)azetidine-1-carboxylate (7.00 g, 26.3 mmol, 1 eq) in DMF (160 mL) was added NaSMe (3.70 g, 52.7 mmol, 2 eq). The mixture was stirred at 25° C. for 3 h. On completion, the mixture was quenched with water (400 mL) and extracted with EA (250 ml×3), the residue was purified by column chromatography (SiO₂, PE/EA=2:1 to 1:1) to give tert-butyl (2S)-2-(methylsulfanylmethyl)azetidine-1-carboxylate (5.60 g, 25.7 mmol, 97% yield) as a yellow oil. ¹H NMR (400 MHz, DMSO-d₆) δ=4.28 (qdd, J=2.8, 5.6, 8.4 Hz, 1H), 3.69 (br d, J=7.6 Hz, 1H), 3.32 (s, 1H), 2.86-2.71 (m, 2H), 2.32-2.20 (m, 1H), 2.08 (s, 3H), 1.92 (tdd, J=6.4, 9.2, 11.2 Hz, 1H), 1.37 (s, 9H)

Step 4. To a solution of tert-butyl (2S)-2-(methylsulfanylmethyl)azetidine-1-carboxylate (5.50 g, 25.3 mmol, 1 eq) in DCM (100 mL) was added m-CPBA (16.3 g, 75.9 mmol, 80% purity, 3 eq). The mixture was stirred at 0° C. for 2 h. On completion, the mixture was quenched with water (100 mL) and extracted with DCM (100 mL×3), the combined organic phase was dried over anhydrous sodium sulfate, filtered and concentrated to give a residue. The residue was purified by column chromatography (SiO₂, PE/EA=2:1 to 1:1) to give tert-butyl (2S)-2-(methylsulfonylmethyl)azetidine-1-carboxylate (6.20 g, 24.8 mmol, 98% yield) as a white solid. ¹H NMR (400 MHz, DMSO-d₆) δ=4.58-4.48 (m, 1H), 3.84-3.72 (m, 2H), 3.64-3.52 (m, 2H), 3.00 (s, 3H), 2.44-2.32 (m, 1H), 2.19 (tdd, J=6.8, 8.8, 11.2 Hz, 1H), 1.38 (s, 9H)

Step 5. To a solution of tert-butyl (2S)-2-(methylsulfonylmethyl)azetidine-1-carboxylate (2.00 g, 8.02 mmol, 1 eq) in DCM (10 mL) was added HCl/dioxane (4 M, 2 mL, 1 eq). The mixture was stirred at 25° C. for 2 h. On completion, the mixture was concentrated to give (2S)-2-(methylsulfonylmethyl)azetidine, 1-39 (1.50 g, crude) as a white solid.

Ex. 139 was prepared in a manner similar to those described in General Method L and in the synthesis of Ex. 131 using I-139 in the appropriate step.

### Preparation of (10S,17E)-8,10,14,16-tetramethyl-12-(oxetan-3-yl)-2,10,11,12,13,14-hexahydro-8H-3,5-ethenotripyrazolo[3,4-f:3′,4′-j:4″,3″-n][1,4]oxazacyclopentadecine (Ex. 140)

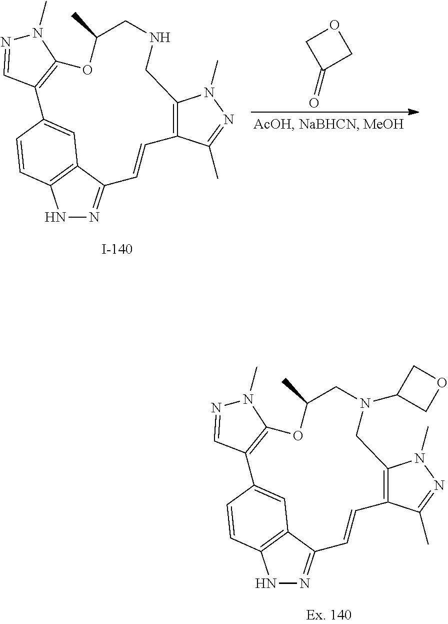 [SMILES: Cc1nn(C)c2c1/C=C/c1n[nH]c3ccc(cc13)-c1cnn(C)c1O[C@@H](C)CNC2 ; O=C1COC1 ; Cc1nn(C)c2c1/C=C/c1n[nH]c3ccc(cc13)-c1cnn(C)c1O[C@@H](C)CN(C1COC1)C2]

(8S,17E)-5,8,13,15-tetramethyl-7-oxa-4,5,10,13,14,20,21-heptazapentacyclo [17.5.2.02,6.012,16.022,26]hexacosa-1(25),2(6),3,12(16),14,17,19, 22(26),23-nonaene, I-140 was prepared via Method I and H using starting materials: G-1-1, H-4-1, and I-2-1 in Ex. 40, 41, and 48, respectively.

Ex. 140 was prepared in a manner like those described in General Method K using oxetan-3-one and I-140.

Ex. 141 was synthesized according to the methods described in General Method P starting with the appropriate alcohol.

Ex. 142 was prepared from 138-2 and 119-2 in a similar manner to those described in the synthesis of Ex. 114 and General Method M.

### Preparation of (10S,17E)-16-{[1-(methanesulfonyl)piperidin-4-yl]oxy}-8,10,12,14-tetramethyl-2,10,11, 12,13,14-hexahydro-8H-3,5-ethenotripyrazolo[3,4-f 3′,4′-j:4″,3″-n][1,4]oxazacyclopentadecine (Ex. 143)

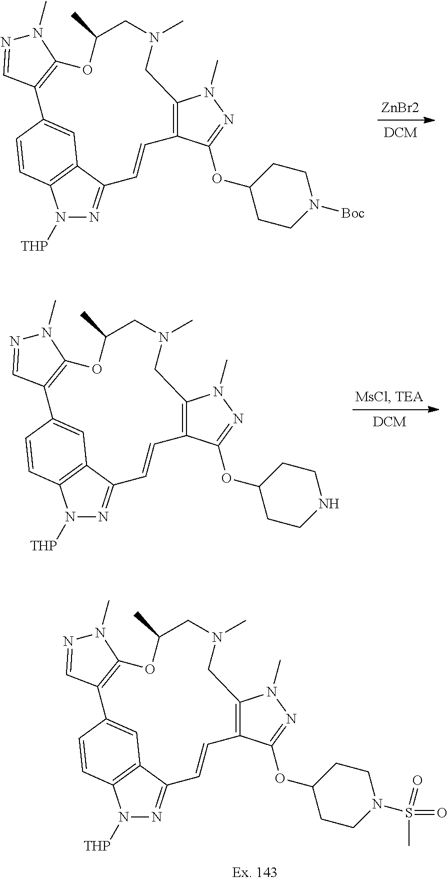

Step 1. To a solution of tert-butyl 4-[[(8S,17E)-5,8,10,13-tetramethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaen-15-yl]oxy]piperidine-1-carboxylate from Ex. 136 (200 mg, 0.291 mmol, 1 eq) in DCM (2 mL) was added ZnBr₂ (197 mg, 0.874 mmol, 3 eq). The mixture was stirred at 25° C. for 16 h. On completion, the mixture was poured into water (20 mL), the aqueous phase was extracted with DCM (5 mL×3). The combined organic phase was washed with brine (10 mL×2), dried over anhydrous Na₂SO₄, filtered and concentrated under vacuum. The residue was purified by column chromatography (SiO₂,DCM:MeOH=10:1) to give (8S,17E)-5,8,10,13-tetramethyl-15-(4-piperidyloxy)-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene (85.0 mg, 0.145 mmol, 50% yield) as a yellow solid. LCMS: m/z 587.4 (M+1)

Step 2. To a solution of (8S,17E)-5,8,10,13-tetramethyl-15-(4-piperidyloxy)-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene (40.0 mg, 0.068 mmol, 1 eq) in DCM (1 mL) was added TEA (10.4 mg, 0.102 mmol, 1.5 eq) and methylsulfonyl methanesulfonate (14.3 mg, 0.082 mmol, 1.2 eq).The mixture was stirred at 25° C. for 0.5 h. On completion, the mixture was concentrated. The residue was purified by prep-HPLC (column: Welch Ultimate C18 150*25 mm*5 um; mobile phase: [water(FA)-ACN]; B %: 15%-45%, 10 min) to give Ex. 143 (2.17 mg, 0.004 mmol, 5% yield) as a white solid.

### Preparation of (10S,13R,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine (Ex. 144) and (10S,13S,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine (Ex. 145)

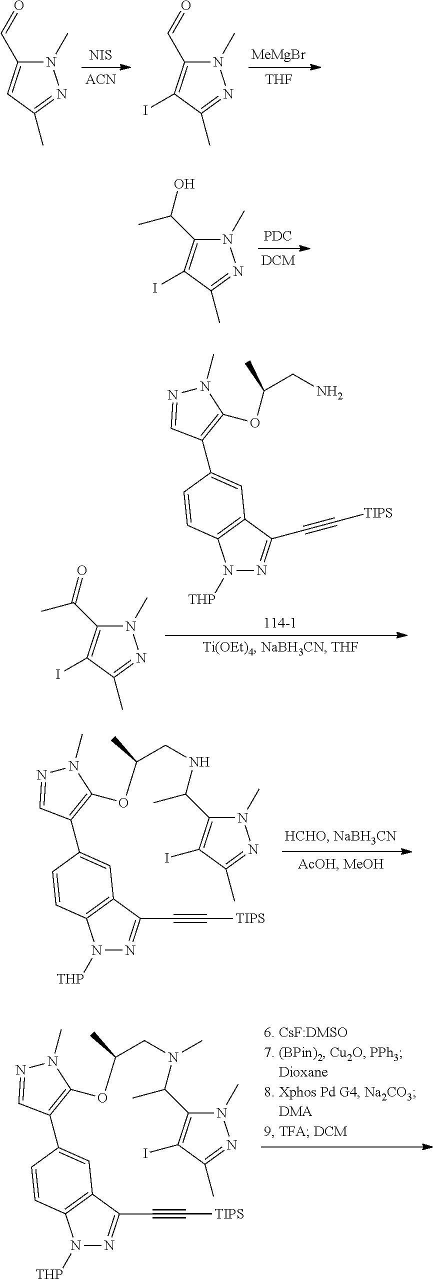

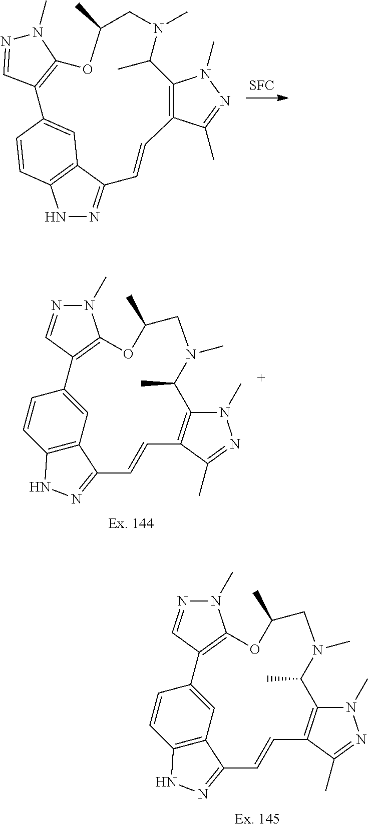

Step 1. To a solution of 2,5-dimethylpyrazole-3-carbaldehyde (1 g, 8.06 mmol, 1 eq) in ACN (10 mL) was added NIS (2.72 g, 12.08 mmol, 1.5 eq).The mixture was stirred at 80° C. for 12 h. On completion, the mixture was concentrated to give a residue. The residue was purified by column chromatography (SiO₂, Petroleum ether/Ethyl acetate=5:1 to 5:1) to give 4-iodo-2,5-dimethyl-pyrazole-3-carbaldehyde (1.62 g, 6.48 mmol, 80% yield) as a brown solid. 1H NMR (400 MHz, DMSO-d₆) δ=9.71 (s, 1H), 4.03 (s, 3H), 2.18 (s, 3H)

Step 2 was performed in similar manner to those described in Ex. I-135

Step 3. To a solution of 1-(4-iodo-2,5-dimethyl-pyrazol-3-yl)ethanol (950 mg, 3.57 mmol, 1 eq) in DCM (30 mL) was added PDC (2.01 g, 5.36 mmol, 1.5 eq) under N₂. The mixture was stirred at 40° C. for 12 h. On completion, the mixture was filtered and the filtrate was concentrated to give a residue. The residue was purified by column chromatography (SiO₂, Petroleum ether/Ethyl acetate=3:1 to 3:1) to give 1-(4-iodo-2,5-dimethyl-pyrazol-3-yl)ethanone (820 mg, 3.11 mmol, 87% yield) as a white solid. 1H NMR (400 MHz, DMSO-d₆) δ=3.95 (s, 3H), 2.68 (s, 3H), 2.18 (s, 3H)

Step 4. To a solution of (2S)-2-[2-methyl-4-[1-tetrahydropyran-2-yl-3-(2-triisopropylsilylethynyl) indazol-5-yl]pyrazol-3-yl]oxypropan-1-amine (430 mg, 0.802 mmol, 1 eq) in THE (6 mL) was added 1-(4-iodo-2,5-dimethyl-pyrazol-3-yl)ethanone (423 mg, 1.61 mmol, 2 eq) and Ti(OEt)₄ (732 mg, 3.21 mmol, 4 eq), the resulting mixture was stirred at 70° C. for 4 h, then NaBH₃CN (302 mg, 4.82 mmol, 6 eq) was added. The mixture was stirred at 25° C. for 12 h. On completion, the reaction mixture was partitioned between ethyl acetate (10 mL×3) and water (10 mL), the combined organic phase was dried over anhydrous sodium sulfate, filtered and concentrated to give a residue. The residue was purified by column chromatography (SiO₂, PE:THF=2:1 to 2:1) to give (2S)—N-[1-(4-iodo-2,5-dimethyl-pyrazol-3-yl)ethyl]-2-[2-methyl-4-[1-tetrahydropyran-2-yl-3-(2-triisopropylsilylethynyl) indazol-5-yl]pyrazol-3-yl]oxy-propan-1-amine (500 mg, crude) as a yellow solid.

Step 5 was conducted in a manner similar to those described in Step 6-9 was conducted in a manner similar to those described in General Method M.

Step 10. The residue was purified by SFC (column: DAICEL CHIRALPAK AD (250 mm*30 mm, 10 um);mobile phase: [Neu-MeOH]; B %:35%, isocratic elution mode) to give arbitrarily assigned Ex. 144 (10.5 mg, 0.024 mmol, 52% yield) as a yellow solid and Ex. 145(4.36 mg, 0.010 mmol, 22% yield) as a yellow solid.

### Preparation of (10R,16E)-3-methyl-25-oxo-3,5,6,9,10,19-hexahydro-8H-20,22-etheno-7,10-methanodipyrazolo[3,4-i:4′,3′-m][1,8,5]benzodioxazacyclooctadecine-14-carbonitrile (Ex. 146)

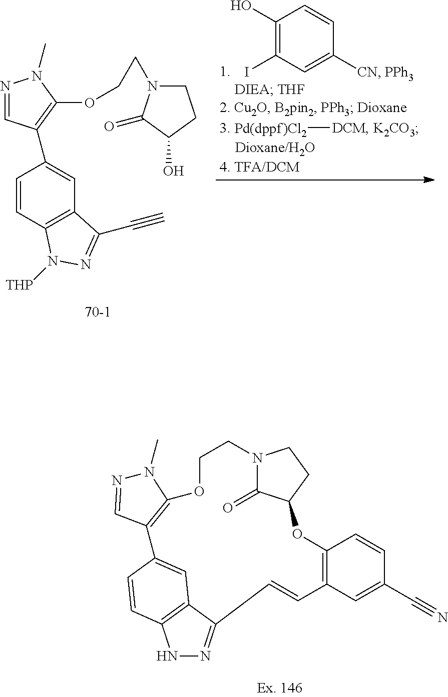

Ex. 146 was prepared in a manner similar to those described in Ex. 131 and General Method L.

### Preparation of 2-{(10S,17E)-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 147)

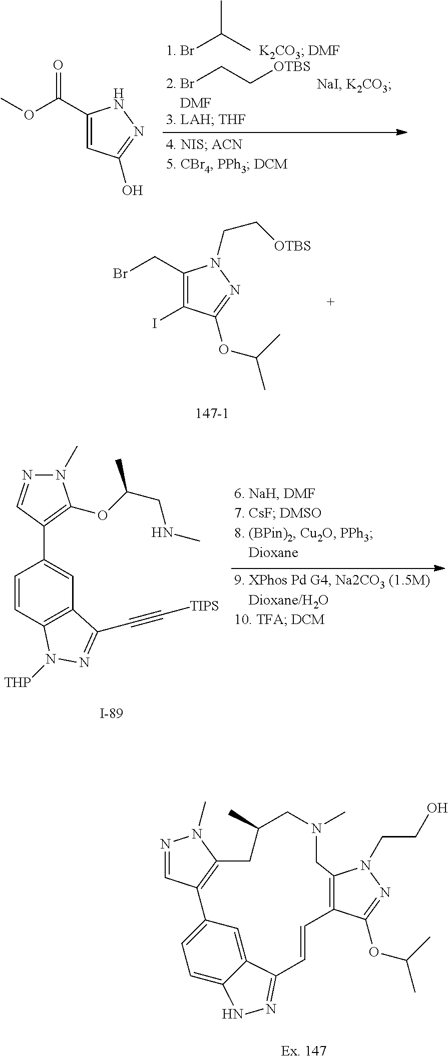

Steps 1-5 were conducted in a similar manner to those described in General Method O.

Steps 6-8 were conducted in a similar manner to those described in General Method H.

Steps 9-10 were conducted in a similar manner to those described in General Method M to afford Ex. 147

### Preparation of (10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-ol (Ex. 148)

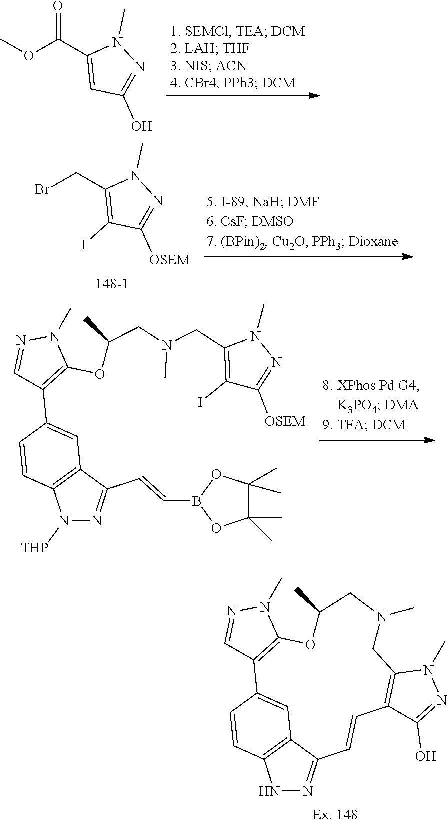

Step 1. To a solution of methyl 5-hydroxy-2-methyl-pyrazole-3-carboxylate (8 g, 51.2 mmol, 1 eq) in DCM (100 mL) was added SEM-C1 (25.6 g, 154 mmol, 3 eq) and TEA (31.1 g, 307 mmol, 6 eq) at 0° C. The mixture was stirred at 25° C. for 16 h. On completion, the reaction mixture was concentrated under reduced pressure to remove solvent. The residue was purified by column chromatography (SiO₂, Petroleum ether/Ethyl acetate=1/0 to 0/1) to give methyl 2-methyl-5-(2-trimethylsilylethoxymethoxy)pyrazole-3-carboxylate (6.43 g, 22.5 mmol, 44% yield) as colorless oil. ¹H NMR (400 MHz, CDCl₃) δ=6.33-6.31 (m, 1H), 5.25-5.24 (m, 2H), 4.06-4.04 (m, 3H), 3.87 (s, 3H), 3.80-3.75 (m, 2H), 1.01-0.95 (m, 2H), 0.01 (s, 9H). LCMS: m/z 287.0 (M+1)

Steps 2-4 were conducted in a manner similar to those described in General Method O.

Steps 5-7 were conducted in a manner similar to those described in General Method H.

Steps 8-9 were conducted in a manner similar to those described in General Method M to afford Ex. 148.

### Preparation of (10S,17E)-16-[(azetidin-3-yl)oxy]-8,10,12,14-tetramethyl-2,10,11,12,13,14-hexahydro-8H-3,5-ethenotripyrazolo[3,4-f:3′,4′-j:4″,3″-n][1,4]oxazacyclopentadecine (Ex. 149)

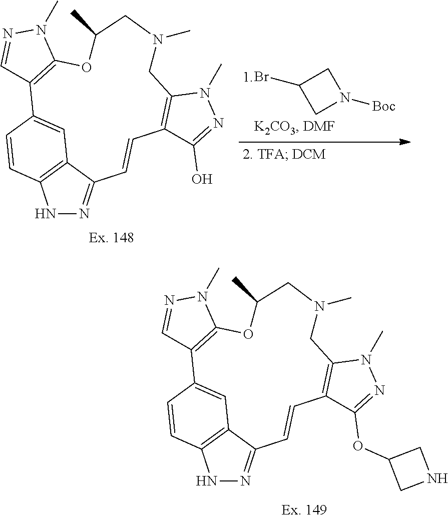 [SMILES: C[C@H]1CN(C)Cc2c(c(O)nn2C)/C=C/c2n[nH]c3ccc(cc23)-c2cnn(C)c2O1 ; CC(C)(C)OC(=O)N1CC(Br)C1 ; C[C@H]1CN(C)Cc2c(c(OC3CNC3)nn2C)/C=C/c2n[nH]c3ccc(cc23)-c2cnn(C)c2O1]

Step 1. To a solution of Ex. 148 (80 mg, 0.191 mmol, 1 eq) in DMF (1 ml) was added K₂CO₃ (79.1 mg, 0.572 mmol, 3 eq) and tert-butyl 3-bromoazetidine-1-carboxylate (90.1 mg, 0.381 mmol, 2 eq). The mixture was stirred at 80° C. for 5 h. On completion, the reaction mixture was filtrated to remove K₂CO₃, and concentrated under reduced pressure to remove solvent. The residue was purified by column chromatography (SiO₂, Petroleum ether/THF=1/0 to 0/1) to give tert-butyl 3-[[(8S,17E)-5,8,10,13-tetramethyl-7-oxa-4,5,10,13,14,20,21-heptazapentacyclo[17.5.2.0^{2,6}.0^{12,16}.0^{22,26}]hexacosa-1(25),2(6),3,12(16),14,17,19,22(26),23-nonaen-15-yl]oxy]azetidine-1-carboxylate (70 mg, 0.122 mmol, 64% yield) as yellow solid. LCMS: m/z 575.3 (M+1)

Step 2. To a solution of tert-butyl 3-[[(8S,17E)-5,8,10,13-tetramethyl-7-oxa-4,5,10,13,14,20,21-heptazapentacyclo[17.5.2.0^{2,6}.0^{12,16}.0^{22,26}]hexacosa-1(25),2(6),3,12(16),14,17,19,22(26),23-nonaen-15-yl]oxy]azetidine-1-carboxylate (18 mg, 0.0313 mmol, 1 eq) in DCM (1 mL) was added TFA (308 mg, 2.70 mmol, 0.2 mL, 86.2 eq). The mixture was stirred at 25° C. for 1 h. On completion, the reaction mixture was concentrated under reduced pressure to remove solvent. The residue was purified by prep-HPLC (column: Phenomenex luna C18 150*25 mm*10 um; mobile phase: [water(FA)-ACN]; B %: 1%-31%, 10 min) to give Ex. 149 (1.23 mg, 0.0023 mmol, 7% yield, 97% purity, FA) as off-white solid.

### Preparation of (10S,17E)-16-{[1-(methanesulfonyl)azetidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine (Ex. 150)

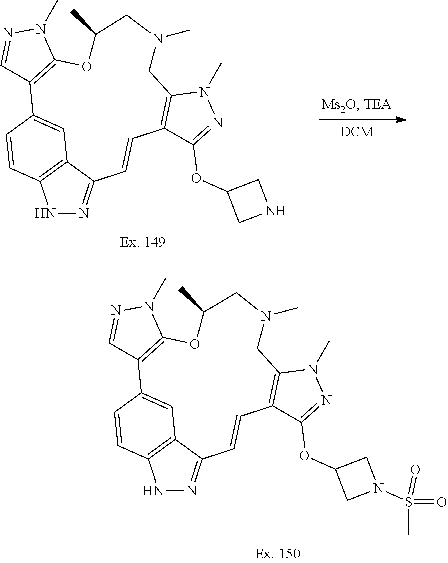 [SMILES: C[C@H]1CN(C)Cc2c(c(OC3CNC3)nn2C)/C=C/c2n[nH]c3ccc(cc23)-c2cnn(C)c2O1 ; C[C@H]1CN(C)Cc2c(c(OC3CN(S(C)(=O)=O)C3)nn2C)/C=C/c2n[nH]c3ccc(cc23)-c2cnn(C)c2O1]

Step 1. To a solution of Ex. 149 (63 mg, 0.133 mmol, 1 eq) in DCM (1 mL) was added TEA (40.3 mg, 0.398 mmol, 55.4 L, 3 eq) and methylsulfonyl methanesulfonate (34.7 mg, 0.199 mmol, 1.5 eq) at 0° C. The mixture was stirred at 25° C. for 1 h. On completion, the residue was purified by prep-HPLC (column: Phenomenex luna C18 150*25 mm*10 um; mobile phase: [water(FA)-ACN]; B %: 15%-45%,10 min) to give Ex. 150 (10.23 mg, 0.018 mmol, 14% yield, 98.29% purity) as white solid.

### Preparation of 2-[(10S,17E)-16-ethoxy-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-o (Ex. 151)

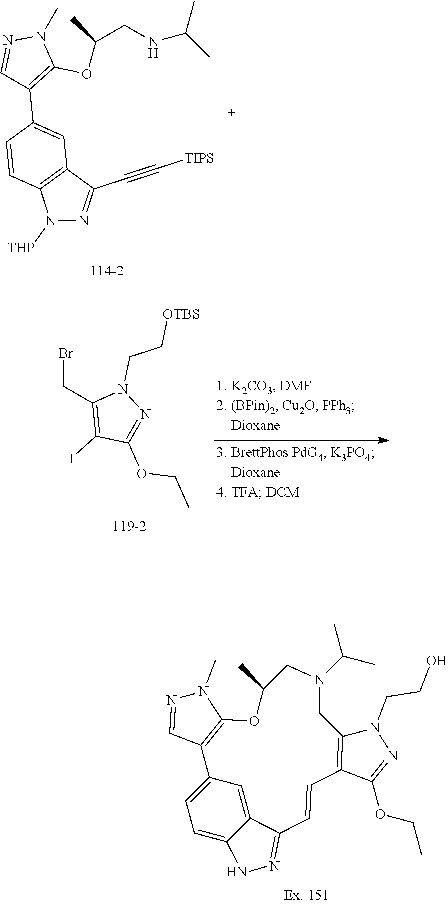

Ex. 151 was prepared in a manner similar to those described in the synthesis of Ex. 114 and General Method Q with starting materials from intermediates of Ex. 114 and General Method O.

### Preparation of 2-[(17E)-10-cyclobutyl-8,12,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 152)

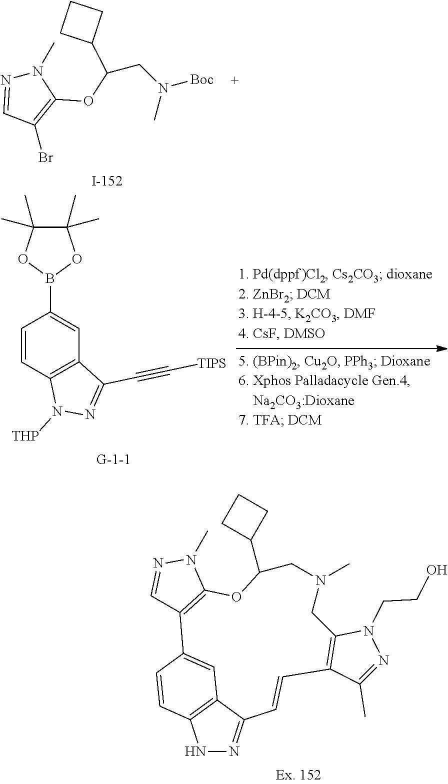

I-152 (General Method M) and G-1-1 were combined in a manner described in General Method I and General Method M to afford Ex. 152.

### Preparation of (10S,17E)-8,10,12,14-tetramethyl-16-{[(3S)-pyrrolidin-3-yl|oxy}-2,10,11,12,13,14-hexahydro-8H-3,5-ethenotripyrazolol3,4-f:3′,4′-j:4″,3″-n][1,4]oxazacyclopentadecine (Ex. 153)

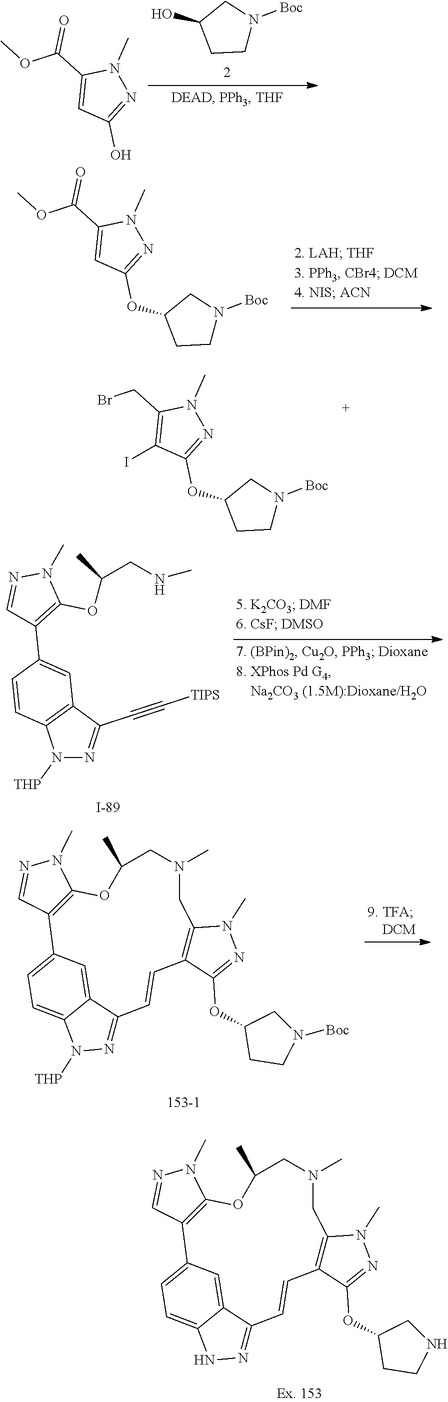

Step 1. A mixture of methyl 5-hydroxy-2-methyl-pyrazole-3-carboxylate (2.5 g, 16.0 mmol, 1.0 eq), tert-butyl (3R)-3-hydroxypyrrolidine-1-carboxylate (6.00 g, 32.0 mmol, 2.0 eq), PPh₃ (8.40 g, 32.0 mmol, 2.0 eq) in THE (20 mL) was degassed and purged with N₂ for 3 times, and then the mixture was stirred at 0° C. for 0.5 h under N₂ atmosphere. Then was added DIAD (6.48 g, 32.0 mmol, 6.23 mL, 2.0 eq) at 0° C. The mixture was stirred at 25° C. for 16 hr under N2 atmosphere. On completion, the mixture was filtered and concentrated to give a residue. The residue was purified by column chromatography (SiO₂, Petroleum ether/Ethyl acetate=1:0 to 2:1) to give methyl 5-[(3S)-1-tert-butoxycarbonylpyrrolidin-3-yl]oxy-2-methyl-pyrazole-3-carboxylate (4.42 g, 13.1 mmol, 82% yield, 97% purity) as a colorless oil. ¹H NMR (400 MHz, CDCl₃) δ=6.15 (s, 1H), 5.11-5.01 (m, 1H), 4.02 (s, 3H), 3.85 (s, 3H), 3.77-3.71 (m, 1H), 3.64-3.53 (m, 2H), 3.50-3.45 (m, 1H), 2.24-2.01 (m, 2H), 1.47-1.44 (m, 9H). LCMS: m/z 270.1 (M−56+1)

Steps 2 to 4 were conducted in a manner similar to those described in General Method 0.

Steps 5-8 were conducted in a manner similar to those described in General Method M.

### Preparation of (10S,17E)-16-{[(3S)-1-(methanesulfonyl)pyrrolidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine (Ex. 154)

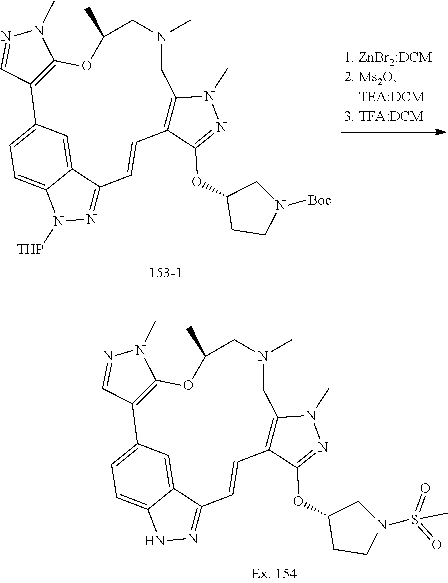

Step 1. To a solution of tert-butyl (3S)-3-[[(8S,17E)-5,8,10,13-tetramethyl-21-tetrahydropyran-2-yl-7-oxa-4,5,10,13,14,20,21-heptazapentacyclo[17.5.2.0^{2,6}.0^{12,16}0.0^{22,26}]hexacosa-1(24),2(6),3,12(16),14,17,19,22,25-nonaen-15-yl]oxy]pyrrolidine-1-carboxylate (40 mg, 0.059 mmol, 1.0 eq) in DCM (2 mL) was added ZnBr₂ (67 mg, 0.297 mmol, 5.0 eq). The mixture was stirred at 25° C. for 16 hr. On completion the mixture was diluted with water (15 mL) and extracted with ethyl acetate (10 mL×3), the combined organic phase was washed with brine dried over anhydrous sodium sulfate, filtered and concentrated in vacuum to give (8S,17E)-5,8,10,13-tetramethyl-15-[(3S)-pyrrolidin-3-yl]oxy-21-tetrahydropyran-2-yl-7-oxa-4,5,10,13,14,20,21-heptazapentacyclo[17.5.2.0^{2,6}.0^{12,16}0.0^{22,26}]hexacosa-1(24),2(6),3,12(16),14,17,19,22,25-nonaene (30 mg, crude) as a colorless oil. LCMS: m/z 573.4 (M+1)

Step 2. To a solution of (8S,17E)-5,8,10,13-tetramethyl-15-[(3S)-pyrrolidin-3-yl]oxy-21-tetrahydropyran-2-yl-7-oxa-4,5,10,13,14,20,21-heptazapentacycl[17.5.2.0^{2,6}.0^{12,16}0.0^{22,26}]hexacosa-1(24),2(6),3,12(16),14,17,19,22,25-nonaene (30 mg, 0.0523 mmol, 1.0 eq) in DCM (1 mL) was added TEA (7.95 mg, 0.079 mmol, 10.9 uL, 1.5 eq) and methylsulfonyl methanesulfonate (10.9 mg, 0.063 mmol, 1.2 eq). The mixture was stirred at 25° C. for 1 hr. The mixture was quenched with water (15 mL) and extracted with ethyl acetate (5 mL×3), the combined organic phase was washed with brine dried over anhydrous sodium sulfate, filtered and concentrated in vacuum to give (8S,17E)-5,8,10,13-tetramethyl-15-[(3S)-1-methylsulfonylpyrrolidin-3-yl]oxy-21-tetrahydropyran-2-yl-7-oxa-4,5,10,13,14,20,21-heptazapentacyclo[17.5.2.0^{2,6}.0^{12,16}.0^{22,26}]hexacosa-1(24),2(6),3,12(16),14,17,19,22,25-nonaene (30 mg, crude) as a colorless oil. LCMS: m/z 651.3 (M+1)

Step 3. To a solution of (8S,17E)-5,8,10,13-tetramethyl-15-[(3S)-1-methylsulfonylpyrrolidin-3-yl]oxy-21-tetrahydropyran-2-yl-7-oxa-4,5,10,13,14,20,21-heptazapentacyclo[17.5.2.0^{2,6}.0^{12,16}.0^{22,26}]hexacosa-1(24),2(6),3,12(16),14,17,19,22,25-nonaene (30 mg, 0.0461 mmol, 1.0 eq) in DCM (2 mL) was added TFA (1.54 g, 13.5 mmol, 1 mL, 292 eq). The mixture was stirred at 25° C. for 2 hr. On completion the mixture was concentrated in vacuum to give a residue. The residue was purified by prep-HPLC (TFA condition; column: Welch Xtimate C18 150*25 mm*5 um; mobile phase: [water(TFA)-ACN]; B %: 12%-42%, 10 min) to give Ex. 154 (120 μg, 2.01e-4 mmol, 4.36e-1% yield, 95% purity) as a off-white gum.

### Preparation of [(2S)-2-[5-(bromomethyl)-3-ethoxy-4-iodo-pyrazol-1-yl]propoxy]-tert-butyl-dimethyl-silane, I-155

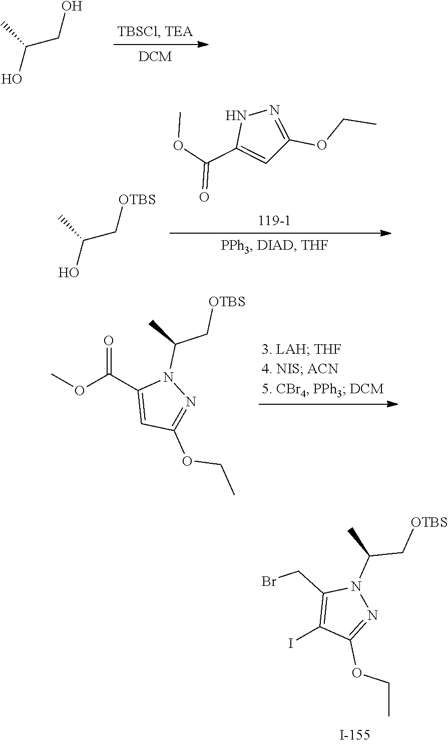

Step 1. To a solution of (2R)-propane-1,2-diol (25 g, 329 mmol, 1 eq), TEA (99.7 g, 986 mmol, 3 eq) in DCM (250 mL) was added TBSCl (52.0 g, 345 mmol, 1.05 eq) at 0° C. The mixture was stirred at 25° C. for 16 hr. On completion, the mixture was concentrated to give a residue. The residue was purified by column chromatography (SiO₂, DCM/MeOH=1:0 to 95:5) to give (2R)-1-[tert-butyl(dimethyl)silyl]oxypropan-2-ol (42 g, 220 mmol, 67% yield) as yellow oil. 1H NMR (400 MHz, CDCl₃) δ=3.85-3.76 (m, 1H), 3.58 (dd, J=3.6, 10.0 Hz, 1H), 3.34 (dd, J=7.6, 9.6 Hz, 1H), 2.53 (d, J=3.2 Hz, 1H), 1.10 (d, J=6.4 Hz, 3H), 0.93-0.85 (m, 9H), 0.10-0.02 (m, 5H).

Step 2. A mixture of methyl 3-ethoxy-1H-pyrazole-5-carboxylate (3 g, 17.6 mmol, 1 eq), (2R)-1-[tert-butyl(dimethyl)silyl]oxypropan-2-ol (7.38 g, 38.8 mmol, 2.2 eq), PPh₃ (10.2 g, 38.8 mmol, 2.2 eq) in THF (30 mL) was degassed and purged with N₂ for 3 times at 0° C. and stirred for 0.5 h, then DIAD (7.84 g, 38.8 mmol, 7.54 mL, 2.2 eq) was added at 0° C., then the mixture was stirred at 25° C. for 1.5 h under N₂ atmosphere. On completion, the reaction mixture was diluted with H₂O (50 mL) and extracted with EA (50 mL*3), dried over Na₂SO₄, filtered and concentrated under reduced pressure to give a residue. The residue was purified by column chromatography (SiO₂, Petroleum ether/Ethyl acetate=1/0 to 0/1) to give methyl 2-[(1S)-2-[tert-butyl(dimethyl)silyl]oxy-1-methyl-ethyl]-5-ethoxy-pyrazole-3-carboxylate (3.2 g, 9.34 mmol, 53% yield) as yellow oil. LCMS: m/z 343.4 (M+1)

Steps 3-5 were conducted in a manner similar to those described in the synthesis of General Method 0 to afford [(2S)-2-[5-(bromomethyl)-3-ethoxy-4-iodo-pyrazol-1-yl]propoxy]-tert-butyl-dimethyl-silane, I-155. LCMS: In/z 503.0 (M+1)

### Preparation of (2S)-2-[(10S,17E)-16-ethoxy-8,10,12-trimethyl-2,8,10,11,12,13-hexahydro-14H-3,5-ethenotripyrazolo|3,4-f:3′,4′-j:4″,3″-n][1,4]oxazacyclopentadecin-14-yl]propan-1-ol (Ex. 155)

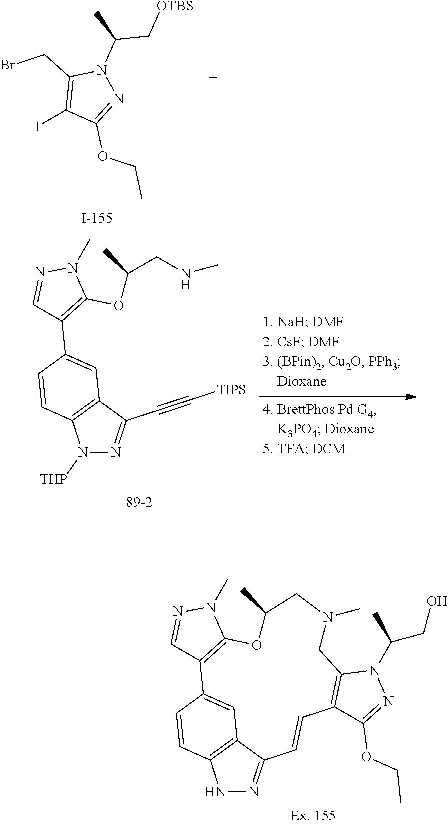

Ex. 155 was prepared with 1-155 and 89-2 in a manner similar to those described in General Method H and General Method Q.

### Preparation of (10S,17E)-16-ethoxy-8,10,14-trimethyl-12-(propan-2-yl)-2,10,11,12,13,14-hexahydro-8H-3,5-ethenotripyrazolo[3,4-f:3′,4′-j:4″,3″-n][1,4]oxazacyclopentadecine (Ex. 156)

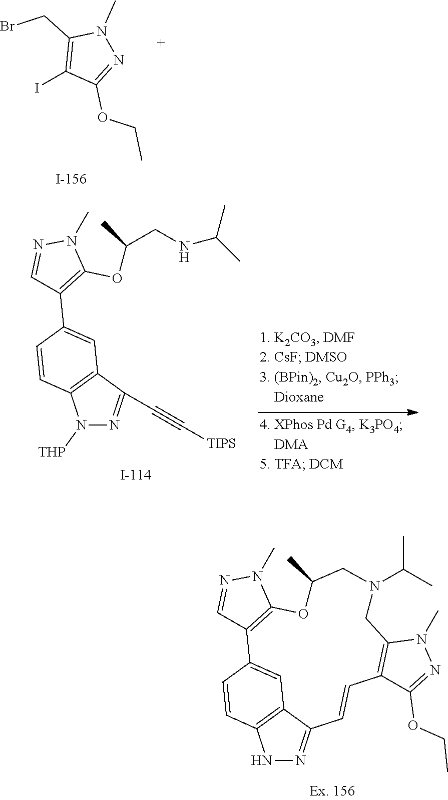

Methyl 5-hydroxy-2-methyl-pyrazole-3-carboxylate was converted to I-156 in a manner similar to those described in General Method O. ¹H NMR (400 MHz, CDCl₃) δ=4.40 (s, 2H), 4.28-4.20 (m, 2H), 3.80 (s, 3H), 1.40 (t, J=7.2 Hz, 3H). LCMS: m/z 346.8 (M+1)

I-156 and 114-2 were combined in a manner similar to those described in General Method M to afford Ex. 156.

### Preparation of (10S,17E)-8,10,14-trimethyl-2,11,12,14-tetrahydro-8H,10H-3,5-etheno[1,5]dioxacyclopentadecino[10,11-c:15,14-c′:6,7-c′]tripyrazole-16-carbonitrile (Ex. 157)

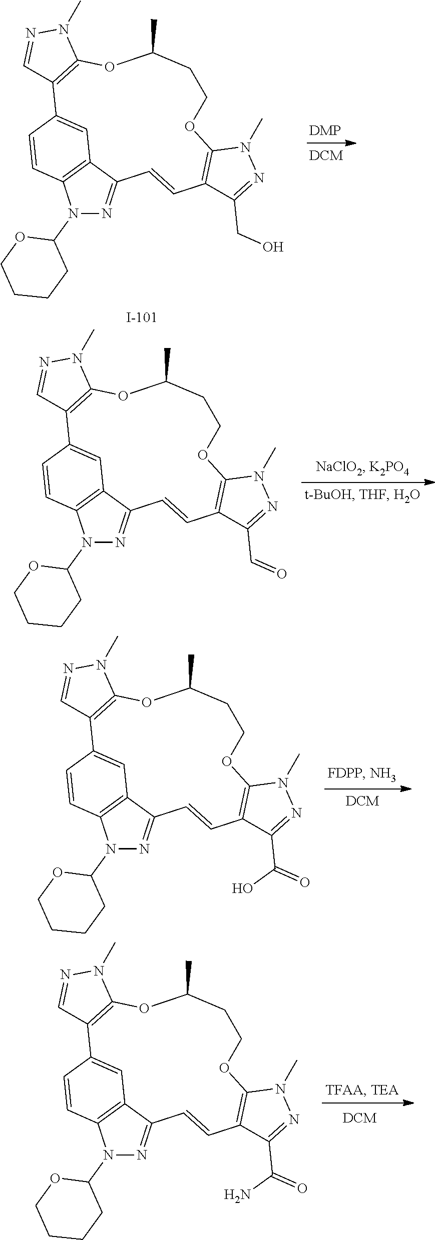

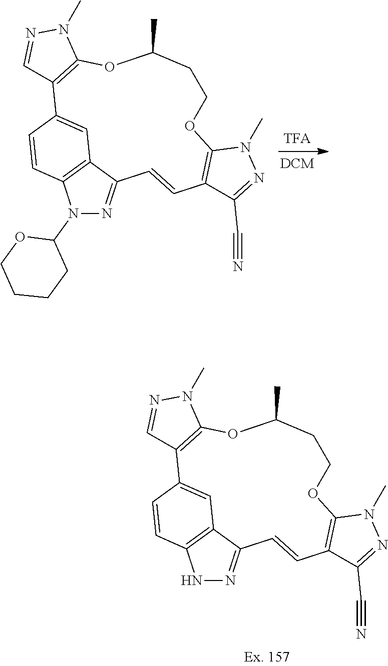 [SMILES: C[C@H]1CCOc2c(c(C#N)nn2C)/C=C/c2nn(C3CCCCO3)c3ccc(cc23)-c2cnn(C)c2O1 ; C[C@H]1CCOc2c(c(C#N)nn2C)/C=C/c2n[nH]c3ccc(cc23)-c2cnn(C)c2O1]

Step 1. To [(13E,25S)-25,30,31-trimethyl-32-tetrahydropyran-2-yl-34,36-dioxa-27,28,29,30,31,32-hexazapentacyclohexacosa-3,5(16),6(27),13,17,19(23),20(24),21(28),22(29)-nonaen-22-yl]methanol (19.87 mg, 0.039 mmol) from Ex. 101 in DCM (1 mL) was added Dess-Martin Periodinane (20 mg, 0.047 mmol) at −78° C., the mixture was allowed to stir and warm to ambient over 18 hr. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 0-10% MeOH in DCM) provided (13E,25S)-25,30,31-trimethyl-32-tetrahydropyran-2-yl-34,36-dioxa-27,28,29,30,31,32-hexazapentacyclohexacosa-3,5(16),6(27),13,17,19(23),20(24),21(28),22(29)-nonaene-22-carbaldehyde (12.53 mg, 0.025 mmol, 63% yield). LCMS: m/z 503.1 (M+1)

Step 2. To a solution of (13E,25S)-25,30,31-trimethyl-32-tetrahydropyran-2-yl-34,36-dioxa-27,28,29,30,31,32-hexazapentacyclohexacosa-3,5(16),6(27),13,17,19(23),20(24),21(28),22(29)-nonaene-22-carbaldehyde (9.2 mg, 0.018 mmol) in tert-butanol (500 μL) was added a solution of 2-methylbut-2-ene (9.63 mg, 0.137 mmol, 15 L) in THF (500 L), followed by sodium chlorite (8.9 mg, 0.099 mmol) and Potassium phosphate dibasic (38 mg, 0.220 mmol) in Water (500 μL). The mixture was stirred at ambient temperature for 18 hr. Reaction was diluted with DCM and 2 M HCl (4 mL) and the layers were separated. The aqueous layer was extracted again with DCM (2×4 mL). The combined organic layer was washed with brine and dried over sodium sulfate. Flash column chromatography (automated system, 12 g silica, 0-10% methanol in DCM with 0.2 mL AcOH added) provided (13E,24S)-24,30,31-trimethyl-32-tetrahydropyran-2-yl-35,37-dioxa-27,28,29,30,31,32-hexazapentacyclohexacosa-3,5(15),6(27),13,16,18(22),19(23),20(28),21(29)-nonaene-21-carboxylic acid (9.49 mg, 0.018 mmol, 100% yield). Taken forward without further purification assuming quantitative yield. LCMS: m/z 519.0 (M+1)

Step 3. To (13E,24S)-24,30,31-trimethyl-32-tetrahydropyran-2-yl-35,37-dioxa-27,28,29,30,31,32-hexazapentacyclohexacosa-3,5(15),6(27),13,16,18(22),19(23),20(28),21(29)-nonaene-21-carboxylic acid (9.2 mg, 0.018 mmol) in DCM (1 mL) was added NH₃ (0.4 M, 887.07 μL) and followed by FDPP (8.18 mg, 0.021 mmol). Stir at 22° C. for 18 hr. Reaction was quenched with 2M Na₂CO₃ (aq) (1 mL) and the mixture was stirred vigorously. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 0-10% MeOH in DCM) provided (13E,24S)-24,31,32-trimethyl-33-tetrahydropyran-2-yl-35,37-dioxa-28,29,30,31,32,33-hexazapentacyclohexacosa-3,5(15),6(28),13,16,18(22),19(23),20(29),21(30)-nonaene-21-carboxamide (1.59 mg, 0.003 mmol, 17% yield). LCMS: m/z 518.3 (M+1)

Step 4. To (13E,24S)-24,31,32-trimethyl-33-tetrahydropyran-2-yl-35,37-dioxa-28,29,30,31,32,33-hexazapentacyclohexacosa-3,5(15),6(28),13,16,18(22),19(23),20(29),21(30)-nonaene-21-carboxamide (1.59 mg, 0.003 mmol) in DCM (200 L) at 0° C. was added TEA (3.73 mg, 0.037 mmol, 5 μL) and TFAA (3.87 mg, 0.018 mmol, 2.6 μL) and stirred for 2 hr, then at RT for a half hour. Quenched with water (10 mL) and extracted with DCM (3×10 mL). Flash column chromatography (automated system, 12 g silica, 0-10% MeOH in DCM) provided (13E,25S)-25,31,32-trimethyl-33-tetrahydropyran-2-yl-34,36-dioxa-28,29,30,31,32,33-hexazapentacyclohexacosa-3,5(16),6(28),13,17,19(23),20(24),21(29),22(30)-nonaene-22-carbonitrile (810.00 μg, 0.002 mmol, 53% yield). LCMS: m/z 500.2 (M+1)

Step 5. To (13E,25S)-25,31,32-trimethyl-33-tetrahydropyran-2-yl-34,36-dioxa-28,29,30,31,32,33-hexazapentacyclohexacosa-3,5(16),6(28),13,17,19(23),20(24),21(29),22(30)-nonaene-22-carbonitrile (810.00 μg, 0.002 mmol) in DCM (1 mL) was added TFA (745.00 mg, 6.53 mmol, 0.5 mL). Stir at 22° C. for 3 hr. Volatiles removed under reduced pressure. Flash column chromatography (automated system, 12 g silica, 0-5% MeOH in DCM) provided Ex. 157 (0.39 mg, 8.24e-4 mmol, 51% yield, 87.8% purity).

### Preparation of 3-(bromomethyl)-4-iodo-5-methyl-isothiazole, I-158

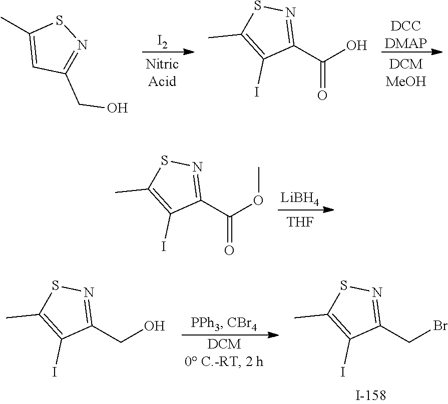

Step 1. To (5-methylisothiazol-3-yl)methanol (300 mg, 2.32 mmol) in concentrated Nitric Acid (209.04 mg, 2.32 mmol, 5 mL, 70% purity) was added Iodine (648 mg, 2.55 mmol). Mixture was stirred for 45 min at 80° C. Cooled to 0° C. and slowly quenched with aq. sodium bisulfite solution (1M, 10 mL). Reaction was diluted with DCM and water (20 mL) and the layers were separated. The aqueous layer was extracted again with DCM (2×10 mL). The combined organic layer was washed with brine and dried over sodium sulfate. Salts were filtered and filtrate was concentrated to approximately 5-10 mL, hexanes (20 mL) was added and solids precipitated. Cooled for several hours and solids filtered and washed with hexanes. Dried under high vacuum to give 4-iodo-5-methyl-isothiazole-3-carboxylic acid (465 mg, 1.73 mmol, 74% yield). LCMS: m/z 269.8 (M+1)

Step 2. To 4-iodo-5-methyl-isothiazole-3-carboxylic acid (465 mg, 1.73 mmol) in DCM (2.5 mL) and methanol (2.5 mL) was added DCC (428 mg, 2.1 mmol), followed by DMAP (21 mg, 0.173 mmol). Stirred at 22° C. for 20 hr. Reaction was diluted with DCM and water (20 mL) and the layers were separated. The aqueous layer was extracted again with DCM (2×10 mL). The combined organic layer was washed with brine and dried over sodium sulfate. Flash column chromatography (automated system, 12 g silica, 0-40% EA in Hexanes) provided methyl 4-iodo-5-methyl-isothiazole-3-carboxylate (347 mg, 1.23 mmol, 71% yield).

To methyl 4-iodo-5-methyl-isothiazole-3-carboxylate (347 mg, 1.23 mmol) in THF (5.86 mL) was added lithium borohydride (53.40 mg, 2.45 mmol). Stirred as temperature increased over 1 hr. Reaction was cooled and quenched with water (5 mL) carefully. Reaction was diluted with DCM and water (30 mL) and the layers were separated. The aqueous layer was extracted again with DCM (2×15 mL). The combined organic layer was washed with brine and dried over sodium sulfate. Flash column chromatography (automated system, 12 g silica, 0-60% EA in Hexanes) provided (4-iodo-5-methyl-isothiazol-3-yl)methanol (222 mg, 0.870 mmol, 71% yield). LCMS: m/z 255.8 (M+1)

Step 4. To (4-iodo-5-methyl-isothiazol-3-yl)methanol (222 mg, 0.870 mmol) in DCM (5 mL) was added triphenylphosphine (410.89 mg, 1.57 mmol). Cooled to 0° C. and carbon tetrabromide (346.4 mg, 1.04 mmol) was added. Stirred for 2 hr as temperature increases to ambient. Quenched with water and worked up with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-40% EA in Hexanes) provided 3-(bromomethyl)-4-iodo-5-methyl-isothiazole, 1-158 (181.54 mg, 0.571 mmol, 66% yield). LCMS: m/z 317.6/319.3 (M/M+2)

Ex. 158 was prepared from I-158 and 89-2 in a manner similar to those described in General Method P and General Method M.

### Preparation of -bromo-5-(bromomethyl)-4-iodo-1-methyl-pyrazole, I-159

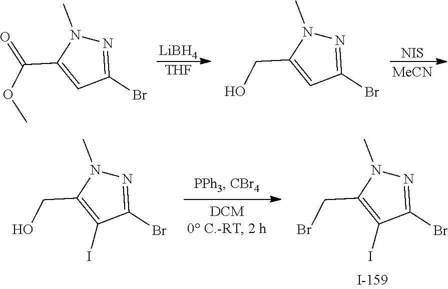

Step 1. Step 1. To methyl 5-bromo-2-methyl-pyrazole-3-carboxylate (500 mg, 2.28 mmol) in THF (11.41 mL) at 0° C. was added, LiBH₄ (74.6 mg, 3.42 mmol). Stirred as temperature increase to RT over 18 hr. An additional 45 mg of LiBH₄ added and the mixture was stirred at ambient temperature for additional 2 hr. Reaction was quenched with water at 0° C. and the reaction was worked up with DCM and water (30 mL). The aqueous layer was extracted with DCM (2×20 mL). The combined organic layer was washed with brine and dried over sodium sulfate. The filtrate was concentrated and residue was crystallized with EA (10 mL) and Hexanes (30 mL) to afford (5-bromo-2-methyl-pyrazol-3-yl)methanol (333 mg, 1.74 mmol, 76% yield). LCMS: m/z 190.9/192.9 (M/M+2)

Step 2. To (5-bromo-2-methyl-pyrazol-3-yl)methanol (333 mg, 1.74 mmol) in acetonitrile (8.7 mL) was added NIS (470.6 mg, 2.09 mmol). Stirred at 22° C. for 18 hr and then quenched with water (5 mL) and worked up with DCM and water (30 mL) and the layers were separated. The aqueous layer was extracted again with DCM (2×20 mL). The combined organic layer was washed with brine and dried over sodium sulfate. Filtrate was concentrated after filtration. Residue was crystallized with EA and Hexanes to afford (5-bromo-4-iodo-2-methyl-pyrazol-3-yl)methanol (488 mg, 1.54 mmol, 88% yield) as a beige solid. LCMS: m/z 316.7/318.7 (M/M+2)

Step 3. To (5-bromo-4-iodo-2-methyl-pyrazol-3-yl)methanol (100 mg, 0.316 mmol) in DCM (2 mL) was added triphenylphosphine (149 mg, 0.568 mmol). Cooled to 0° C. and carbon tetrabromide (125.6 mg, 0.379 mmol) was added. Stirred as temperature increases to ambient for 1.5 h. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 0-30% EA in Hexanes) provided -bromo-5-(bromomethyl)-4-iodo-1-methyl-pyrazole, 1-159 (68.24 mg, 0.180 mmol, 57% yield). LCMS: m/z 377.7 (M−1)

### Preparation of (10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecine-16-carbonitrile (Ex. 159)

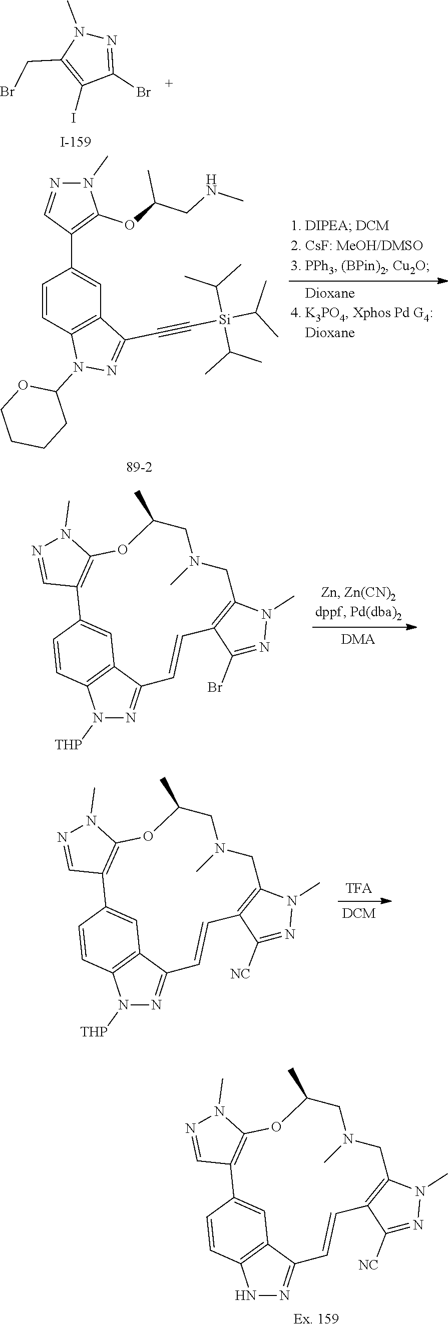

Steps 1-4 were prepared from I-159 and 89-2 in a manner similar to those described in General Method P and General Method M.

Step 5. To (14E,25S)-23-bromo-25,30,31,33-tetramethyl-32-tetrahydropyran-2-yl-35-oxa-27,28,29,30,31,32,33-heptazapentacyclohexacosa-4,6(16),7(27),14,17,19(24),20,22(28),23(29)-nonaene (9.76 mg, 0.017 mmol) in DMA (1 mL) was added zinc (2.3 mg, 0.035 mmol) and zinc cyanide (22.3 mg, 0.190 mmol), argon was bubbled through as dppf (5.73 mg, 0.010 mmol) was added followed by Pd(dba)₂ (2.97 mg, 0.0052 mmol). Stir under argon for about 5 minutes. The vessel was closed and heated to 120° C. and stirred for 6 hr. Reaction was diluted with DCM and water (10 mL) and the layers were separated. The aqueous layer was extracted again with DCM (2×10 mL). The combined organic layer was washed with brine and dried over sodium sulfate. Flash column chromatography twice (automated system, 12 g silica, 0-10% MeOH in DCM, then 0-10% MeOH/DCM (1/3) in EA) provided (14E,26S)-26,32,33,35-tetramethyl-34-tetrahydropyran-2-yl-37-oxa-29,30,31,32,33,34,35-heptazapentacyclohexacosa-4,6(17),7(29),14,18,20(25),21,23(30),24(31)-nonaene-24-carbonitrile (3.85 mg, 0.008 rmnol, 44% yield). LCMS: m/z 513.0 (M+1)

Step 6. To (14E,26S)-26,32,33,35-tetramethyl-34-tetrahydropyran-2-yl-37-oxa-29,30,31,32,33,34,35-heptazapentacyclohexacosa-4,6(17),7(29),14,18,20(25),21,23(30),24(31)-nonaene-24-carbonitrile (3.85 mg, 0.008 mmol) in dem (1 mL) was added TFA (6.53 mmol, 0.5 mL). Stir at 22° C. for 18 hr. Volatiles removed under reduced pressure. Sample re-diluted with DCM and TEA (200 uL) added. Flash column chromatography (automated system, 12 g silica, 0-10% MeOH in DCM) provided Ex. 159 (1.85 mg, 0.004 mmol, 53% yield, 92.77% purity).

Ex. 160 was prepared with 122-1 and I-156 in a similarly to Ex. 122.

Ex. 161 was prepared in a manner similar to those described in General Method K using (1-ethoxycyclopropoxy)-trimethyl-silane and 1-140.

### General Method R. Preparation of (10S,17E)-8,10,12,14-tetramethyl-16-(2,2,2-trifluoroethoxy)-2,10,11,12,13,14-hexahydro-8H-3,5-ethenotripyrazolo[3,4-f:3′,4′-j:4″,3″-n][1,4]oxazacyclopentadecine (Ex. 162) and {[(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile (Ex. 163)

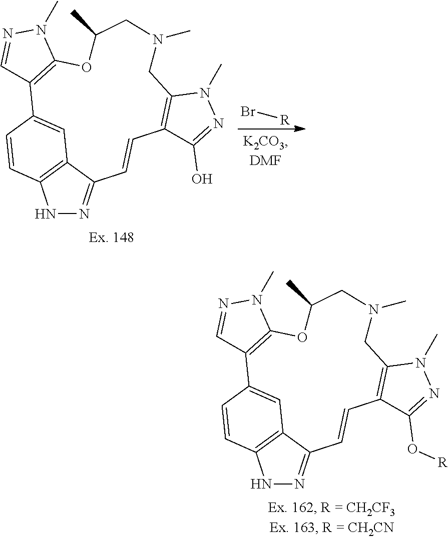

Ex. 148 was converted to Ex. 162 and 163 in the same fashion using the corresponding bromide.

Step 1. To a solution of (8S,17E)-5,8,10,13-tetramethyl-7-oxa-4,5,10,13,14,20,21-heptazapentacyclo[17.5.2.02,6.012,16.022,26]hexacosa-1(25),2(6),3,12(16),14,17,19,22(26),23-nonaen-15-ol (20 mg, 0.048 mmol, 1 eq) in DMF (0.5 mL) was added 2-bromo-1,1,1-trifluoro-ethane (15.5 mg,0.095 mmol, 2 eq) and K₂CO₃ (19.8 mg, 0.143 mmol, 3 eq). The mixture was stirred at 80° C. for 3 h. On completion, the mixture was filtrated to remove K₂CO₃. The solvent was purified by prep-HPLC (column: Phenomenex luna C18 150*25 mm*10 um; mobile phase: [water(FA)-ACN]; B %: 28%-58%,10 min) to give Ex. 162 (3.58 mg, 0.007 mmol, 13.71% yield, FA) as yellow solid.

### Preparation of (10S,17E)-10,14,16-trimethyl-8-[(methylsulfanyl)methyl]-12-(propan-2-yl)-2,10,11,12,13,14-hexahydro-8H-3,5-ethenotripyrazolo[3,4-f:3′,4′-j:4″,3″-n][1,4]oxazacyclopentadecine (Ex. 164)

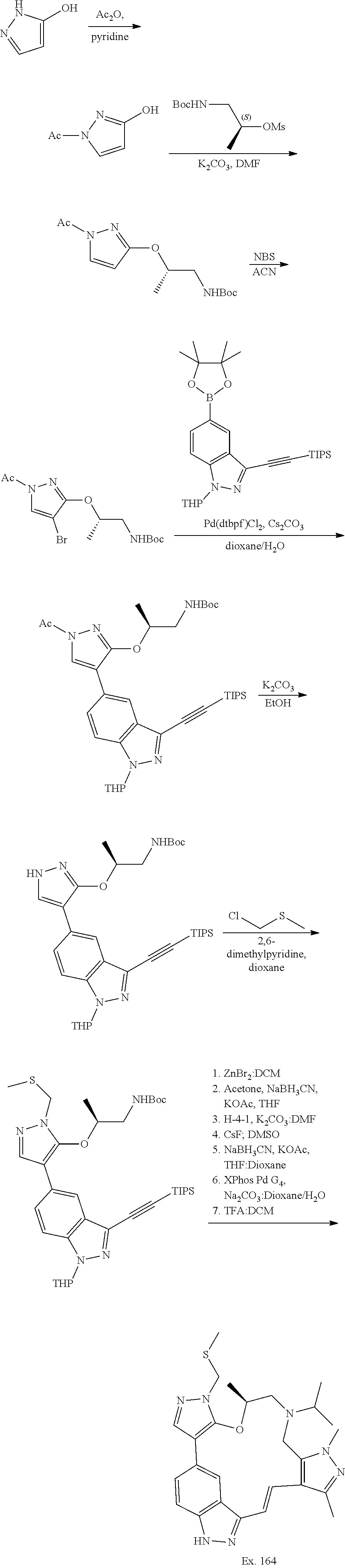

Step 1. The mixture of 1H-pyrazol-5-ol (10.0 g, 118 mmol, 1.00 eq) in Py (100 mL) was stirred at 95° C. for 0.5 h, then acetyl acetate (12.1 g, 118 mmol, 11.1 mL, 1.00 eq) was dissolve in Py (30.0 mL) was added in the reaction mixture., the mixture was stirred at 95° C. for 2.5 h. On completion, the reaction mixture was filtered and concentrated under reduced pressure to give a residue. The crude product was triturated with PE (100 mL) at 30° C. for 10 min to give 1-(3-hydroxypyrazol-1-yl)ethanone (14.8 g, 114 mmol, 96% yield, 97% purity) as a light yellow solid. LCMS: m/z 149.1(M+23)

Step 2. To a mixture of 1-(3-hydroxypyrazol-1-yl)ethanone (10.0 g, 79.3 mmol, 1.00 eq) and [(1S)-2-(tert-butoxycarbonylamino)-1-methyl-ethyl]methanesulfonate (22.1 g, 87.2 mmol, 1.10 eq) in DMF (200 mL) was added K₂CO₃ (32.9 g, 238 mmol, 3.00 eq). The mixture was stirred at 80° C. for 16 h. On completion, the reaction mixture was concentrated under vacuum. The residue was purified by flash silica gel chromatography (ISCO®; 120 g SepaFlash® Silica Flash Column, Eluent of 0-30% Ethyl acetate/Petroleum ether gradient @100 mL/min) to give tert-butyl N-[(2S)-2-(1-acetylpyrazol-3-yl) oxypropyl]carbamate (49.0 g, 77.8 mmol, 98% yield, 45% purity) as a light yellow oil. LCMS: m/z 306.1 (M+23)

Step 3. To a solution of tert-butyl N-[(2S)-2-(1-acetylpyrazol-3-yl)oxypropyl]carbamate (25.0 g, 88.2 mmol, 1.00 eq) in ACN (150 ml) was added NBS (7.85 g, 44.1 mmol, 0.50 eq) at 0° C. Then the mixture was stirred at 0° C. for 1 h. On completion, the mixture was quenched by Na₂SO₃ (100 mL) and extracted with EtOAc (40 mL*3). The combined organic layer was washed with brine (100 mL*2), dried over anhydrous Na₂SO₄, filtered and the filtrate was concentrated in vacuum to give crude. The residue was purified by combi flash (100 g silica gel column, EtOAc in PE from 0% to 100%) to give tert-butyl N-[(2S)-2-(1-acetyl-4-bromo-pyrazol-3-yl)oxypropyl]carbamate (5.70 g, 15.7 mmol, 18% yield) as brown oil. LCMS: m/z 385.9 (M+23)

Step 4 was conducted in a manner similar to those described in General Method I with G-1-1. LCMS: m/z 664.5 (M+1)

Step 5. To a solution of tert-butyl N-[(2S)-2-[1-acetyl-4-[1-tetrahydropyran-2-yl-3-(2-triisopropylsilylethynyl) indazol-5- yl]pyrazol-3-yl]oxypropyl]carbamate (2.40 g, 3.61 mmol, 1.00 eq) in EtOH (1 mL) was added K₂CO₃ (1.50 g, 10.8 mmol, 3.00 eq). The mixture was stirred at 25° C. for 2 h. On completion, the mixture was concentrated in vacuum to give a residue. The residue was purified by combi flash (40 g silica gel column, EtOAc in PE from 0% to 100%) to give tert-butyl N-[(2S)-2-[[4-[1-tetrahydropyran-2-yl-3-(2-triisopropylsilylethynyl) indazol-5-yl]-1H-pyrazol-3- yl]oxy]propyl]carbamate (1.90 g, 3.06 mmol, 85% yield) as brown oil. LCMS: m/z 622.5 (M+1)

Step 6. To a solution of tert-butyl N-[(2S)-2-[[4-[1-tetrahydropyran-2-yl-3-(2-triisopropylsilylethynyl) indazol-5-yl]-1Hpyrazol-3-yl]oxy]propyl]carbamate (3.80 g, 6.11 mmol, 1.00 eq) and chloro(methylsulfanyl)methane (2.95 g, 30.6 mmol, 2.57 mL, 5.00 eq) in dioxane (40 mL) was added 2,6-dimethylpyridine (1.31 g, 12.2 mmol, 1.42 mL, 2.00 eq). The mixture was stirred at 110° C. for 4 h. On completion, the mixture was concentrated in vacuum to give crude. The residue was purified by combi flash (40 g silica gel column, EtOAc in PE from 0% to 100%) to give tert-butyl N-[(2S)-2-[2-(methylsulfanylmethyl)-4-[1-tetrahydropyran-2-yl-3-(2-triisopropylsilylethynyl) indazol-5- yl]pyrazol-3-yl]oxypropyl]carbamate (2.10 g, 3.08 mmol, 50% yield) as brown oil. ¹H NMR (400 MHz, DMSO-d₆) δ=7.96-7.89 (m, 1H), 7.87-7.80 (m, 2H), 7.76 (d, J=8.8 Hz, 1H), 7.13-7.01 (m, 1H), 6.02-5.91 (m, 1H), 5.30-5.18 (m, 2H), 4.29-4.18 (m, 1H), 3.89-3.79 (m, 1H), 3.29-3.14 (m, 2H), 2.66-2.56 (m, 3H), 2.52-2.39 (m, 1H), 1.92-1.75 (m, 1H), 1.73-1.62 (m, 2H), 1.24-1.21 (m, 13H), 1.20-1.11 (m, 21H). LCMS: m/z 682.5 (M+1)

tert-butyl N-[(2S)-2-[2-(methylsulfanylmethyl)-4-[1-tetrahydropyran-2-yl-3-(2-triisopropylsilylethynyl) indazol-5- yl]pyrazol-3-yl]oxypropyl]carbamate was converted to Ex. 164 in a manner similar to those described in General Method M.

Ex. 165 was prepared in a manner similar to those described in General Method K using acetaldehyde and I-140.

### Preparation of (10S,20E)-18-[2-(methanesulfonyl)ethyl]-8,10,12-trimethyl-2,8,10,11,12,13,16,17,18,19-decahydro-3,5-ethenopyrazino[1′,2′:1,5]pyrazolo [3,4-f]dipyrazolo[3,4-j:4′,3′-n][1,4]oxazacyclopentadecine (Ex. 166)

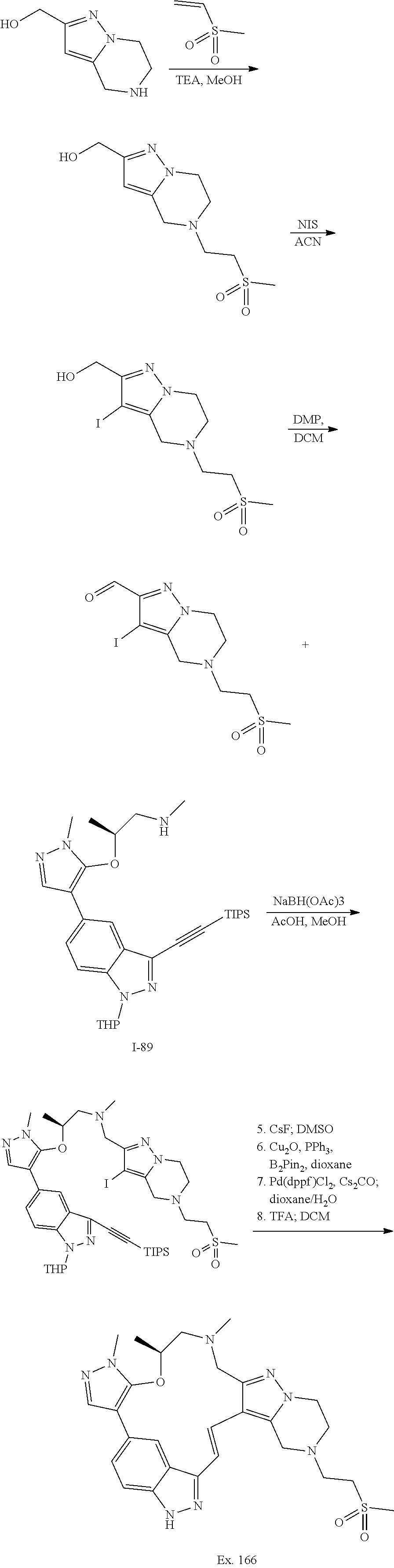

Step 1. The reaction mixture of 4,5,6,7-tetrahydropyrazolo[1,5-a]pyrazin-2-ylmethanol (1.00 g, 6.53 mmol, 1 eq) and 1-methylsulfonylethylene (2.08 g, 19.5 mmol, 1.72 mL, 3 eq) in EtOH (7 mL) was stirred at 60° C. for 1 hour. On completion, the reaction mixture was filtered and the filtrate was concentrated in vacuo to afford [5-(2-methylsulfonylethyl)-6,7-dihydro-4H-pyrazolo[1,5-a]pyrazin-2-yl]methanol (1.30 g, 5.01 mmol, 76% yield) as white solid. ¹H NMR (400 MHz, DMSO-d₆) δ 5.94 (s, 1H), 4.93 (t, J=5.6 Hz, 1H), 4.34 (d, J=5.2 Hz, 2H), 4.00 (t, J=5.2 Hz, 2H), 3.68 (s, 2H), 3.00 (s, 3H), 2.97-2.89 (m, 4H), 2.50 (s, 2H)

Step 2. The reaction mixture of [5-(2-methylsulfonylethyl)-6,7-dihydro-4H-pyrazolo[1,5-a]pyrazin-2-yl]methanol (1.30 g, 5.01 mmol, 1 eq) in ACN (15 mL) was add NIS (1.35 g, 6.02 mmol, 1.2 eq). The reaction mixture was stirred at 25° C. for 1 hour. On completion, the reaction mixture was quenched with sat. NaS₂O₃ (8 mL), diluted with water (30 mL) and extracted with EA (2×30 mL). The combined organic layers was dried over Na₂SO₄, filtered and concentrated in vacuo to give [3-iodo-5-(2-methylsulfonylethyl)-6,7-dihydro-4H-pyrazolo[1,5-a]pyrazin-2-yl]methanol (1.40 g, 3.63 mmol, 73% yield) as white solid. ¹H NMR (400 MHz, DMSO-d₆) δ=4.95 (t, J=5.4 Hz, 1H), 4.30 (d, J=5.6 Hz, 2H), 4.05 (t, J=5.2 Hz, 2H), 3.56 (s, 2H), 3.43 (t, J=6.8 Hz, 2H), 3.01 (s, 3H), 3.00-2.93 (m, 4H). LCMS: m/z 385.9 (M+1)

Step 3. To a solution of [3-iodo-5-(2-methylsulfonylethyl)-6,7-dihydro-4H-pyrazolo[1,5-a]pyrazin-2-yl]methanol (1 g, 2.60 mmol, 1 eq) in DCM (15 mL) was added DMP (1.65 g, 3.89 mmol, 1.21 mL, 1.5 eq). The reaction mixture was stirred at 25° C. for 1 hour. On completion, the reaction mixture was quenched with sat. NaS₂O₃ (8 mL) and sat. NaHCO₃ (8 mL). The reaction mixture was diluted with water (30 mL) and extracted with EA (2×30 mL). The combined organic layers was dried over Na₂SO₄, filtered and concentrated in vacuo to give a residue. The residue was purified by column chromatography (SiO₂, PE: EA=50:1 to PE: EA=0:1) to afford 3-iodo-5-(2-methylsulfonylethyl)-6,7-dihydro-4H-pyrazolo[1,5-a]pyrazine-2-carbaldehyde (1.50 g, crude) as white solid. ¹H NMR (400 MHz, DMSO-d₆) δ=9.86-9.80 (m, 1H), 4.24 (t, J=5.4 Hz, 2H), 3.66 (s, 2H), 3.44 (t, J=6.8 Hz, 2H), 3.07-3.02 (m, 4H), 3.01 (s, 3H). LCMS: m/z 383.8 (M+1)

Step 4. The reaction mixture of (2S)—N-methyl-2-[2-methyl-4-[1-tetrahydropyran-2-yl-3-(2-triisopropylsilylethynyl) indazol-5-yl]pyrazol-3-yl]oxy-propan-1-amine (1.51 g, 2.74 mmol, 0.75 eq in THF (8 mL) was added TEA (370 mg, 3.65 mmol, 508 μL, 1 eq). Then 3-iodo-5-(2-methylsulfonylethyl)-6,7-dihydro-4H-pyrazolo[1,5-a]pyrazine-2-carbaldehyde (1.4 g, 3.65 mmol, 1 eq) and HOAc (219 mg, 3.65 mmol, 1 eq) was added to the reaction mixture above. The reaction mixture was stirred at 25° C. for 0.5 hour. Then NaBH(OAc)₃ (1.16 g, 5.48 mmol, 1.5 eq) was added. The reaction mixture was stirred at 25° C. for 1 hour. On completion, the residue was diluted with water (30 mL) and extracted with EA (2×30 mL). The combined organic layers was dried over Na₂SO₄, filtered and concentrated in vacuo to give a residue. The residue was purified by column chromatography (SiO₂, PE:EA=50:1 to PE:EA=0:1) to give (2S)—N-[[3-iodo-5-(2-methylsulfonylethyl)-6,7-dihydro-4H-pyrazolo[1,5-a]pyrazin-2-yl]methyl]-N-methyl-2-[2-methyl-4-[1-tetrahydropyran-2-yl-3-(2-triisopropylsilylethynyl) indazol-5-yl]pyrazol-3-yl]oxy-propan-1-amine (1.23 g, 1.34 mmol, 37% yield) as yellow gum. ¹H NMR (400 MHz, DMSO-d₆) δ=7.82 (d, J=8.9 Hz, 1H), 7.78 (s, 1H), 7.70-7.65 (m, 2H), 5.92-5.86 (m, 1H), 4.22-4.13 (m, 1H), 4.02-3.97 (m, 2H), 3.90 (br d, J=10.6 Hz, 1H), 3.80-3.74 (m, 1H), 3.69 (s, 3H), 3.52 (s, 2H), 3.43-3.41 (m, 2H), 3.30 (br s, 3H), 3.00 (s, 4H), 2.98-2.92 (m, 4H), 2.67 (br dd, J=5.3, 12.8 Hz, 1H), 2.42-2.31 (m, 2H), 2.07-2.04 (m, 3H), 1.80-1.70 (m, 1H), 1.59 (br s, 2H), 1.18-1.14 (m, 24H). LCMS: m/z 917.2 (M+1)

Steps 5-8 were conducted in a similar manner to those described in General Method H to afford Ex. 166

Ex. 167 was prepared in a similar manner to those described in General Method M with tert-butyl (S)-3-((methylsulfonyl)oxy)pyrrolidine-1-carboxylate as starting material and intermediates G-1-1, and H-4-2 where appropriate.

### Preparation of (2S)-2-[2-ethyl-4-[1-tetrahydropyran-2-yl-3-(2-triisopropylsilylethynyl) indazol-5-yl]pyrazol-3-yl]oxy-N-methyl-propan-1-amine (I-168)

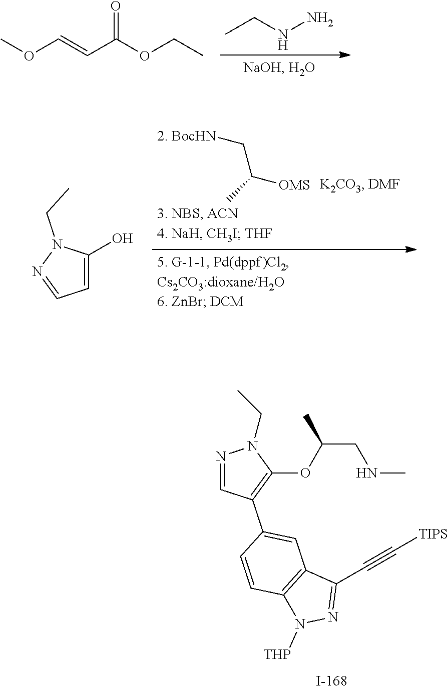

Step 1. To a mixture of ethyl (E)-3-ethoxyprop-2-enoate (1 g, 6.94 mmol, 1 eq) and ethylhydrazine;oxalic acid (1.04 g, 6.94 mmol, 1 eq) in water (20 mL) was added NaOH (277 mg, 6.94 mmol, 1 eq). The mixture was stirred at 50° C. for 2 hours. On completion, the mixture was cooled to 20° C. and extracted with DCM/i-Pr—OH (20 mL×3), the combined organic phase was dried over anhydrous sodium sulfate, filtered and concentrated to give 2-ethylpyrazol-3-ol (700 mg, 6.24 mmol, 90% yield) as a yellow solid. ¹H NMR (400 MHz, DMSO-d₆) δ=7.52 (d, J=12.4 Hz, 1H), 5.14 (d, J=12.4 Hz, 1H), 3.98-3.89 (m, 2H), 1.27-1.21 (m, 3H).

Steps 2-4 were conducted according to General Method M.

Step 5 was conducted according to General Method I.

Step 6. To a solution of tert-butyl N-[(2S)-2-[2-ethyl-4-[1-tetrahydropyran-2-yl-3-(2-triisopropylsilylethynyl) indazol-5-yl]pyrazol-3-yl]oxypropyl]-N-methyl-carbamate (0.72 g, 1.08 mmol, 1 eq) in DCM (10 mL) was added ZnBr₂ (1.47 g, 6.51 mmol, 6 eq). The mixture was stirred at 25 C for 14 hours. On completion, the reaction mixture was partitioned between DCM (10 mL×3) and water (10 mL), the combined organic phase was dried over anhydrous sodium sulfate, filtered and concentrated to give a residue. The residue was purified by column chromatography (SiO₂, Dichloromethane/Methanol=1:0 to 0:1) to give (2S)-2-[2-ethyl-4-[1-tetrahydropyran-2-yl-3-(2-triisopropylsilylethynyl) indazol-5-yl]pyrazol-3-yl]oxy-N-methyl-propan-1-amine (400 mg, 0.709 mmol, 65% yield) as yellow oil. ¹H NMR (400 MHz, CDCl₃) δ=7.83-7.76 (m, 1H), 7.65-7.57 (m, 3H), 5.76-5.68 (m, 1H), 4.36-4.24 (m, 1H), 4.13-4.09 (m, 2H), 3.85-3.57 (m, 2H), 2.89-2.67 (m, 2H), 2.64-2.36 (m, 5H), 2.05 (s, 3H), 1.78-1.73 (m, 3H), 1.70-1.64 (m, 2H), 1.50 (t, J=7.2 Hz, 3H), 1.21-1.17 (m, 21H).

Ex. 168 was prepared from 1-168 and 119-2 in a similar manner to those described in General Method M.

Ex. 169 was prepared from I-168 and 147-1 in a similar manner to those described in General Method M.

### Preparation of (10R,16E)-3-methyl-3,5,6,9,10,19-hexahydro-8H-20,22-etheno-7,10-methanodipyrazolo[3,4-i:4′,3′-m][1,8,5]benzodioxazacyclooctadecine (Ex. 170)

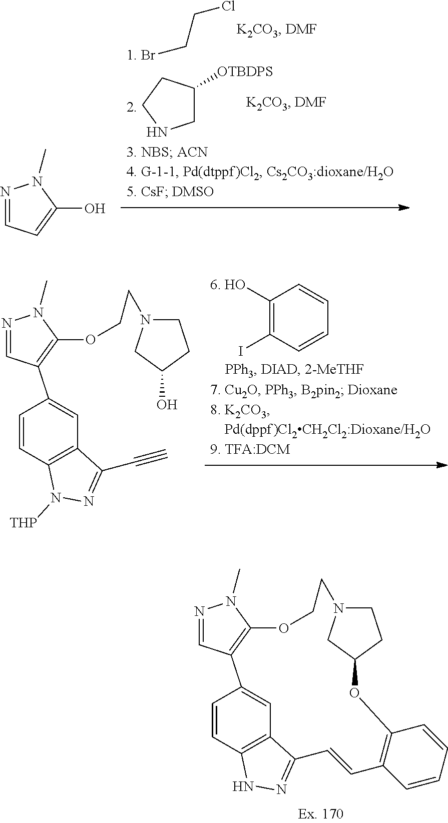

Steps 1-5 are described in General Method M.

Steps 6-9 were conducted in similar manner to those described in General Method L and General Method H to afford Ex. 170.

Ex. 171 was prepared as described in the synthesis for Ex. 155 starting with (2S)-propane-1,2-diol instead.

Ex. 172 was prepared in manner similar to those described in General Method M from 116-1 and 119-2

Ex. 173 and Ex. 174 were prepared in a manner described in General Method P starting from appropriate alcohol, (3-cyclopropylisoxazol-5-yl)methanol and (3-ethylisoxazol-5-yl)methanol, respectively.

### Preparation of [2-[2-[tert-butyl(dimethyl) silyl]oxyethyl]-4-iodo-5-[(1-methylsulfonyl-4-piperidyl)oxy]pyrazol-3-yl]methyl methanesulfonate, (I-175)

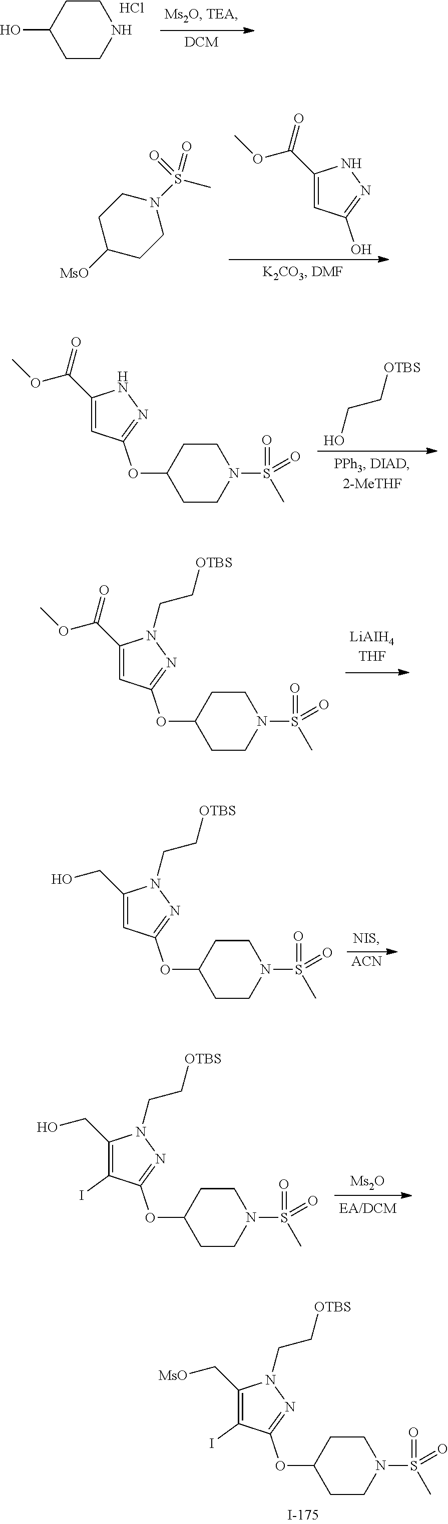

Step 1. To a mixture of piperidin-4-ol (5.00 g, 36.3 mmol, 1 eq, HCl), TEA (11.0 g, 109 mmol, 3 eq) in DCM (50 mL) was added bis(methylsulfonyl)methane (9.39 g, 54.5 mmol, 1.5 eq). The mixture was stirred at 25° C. for 12 hours. On completion, the mixture was quenched by water (100 mL), and extracted with DCM (50 mL*2), The combined organic layers were washed with aq. NaCl (50 mL*2), filtered and concentrated under reduced pressure to give 1-(methylsulfonyl)piperidin-4-yl methanesulfonate (6.75 g, 26.2 mmol, 53% yield) as a brown oil. ¹H NMR (400 MHz, DMSO-d₆) δ=4.85-4.82 (m, 1H), 3.28-3.26 (m, 1H), 3.21 (s, 3H), 3.17-3.14 (m, 2H), 2.83 (s, 3H), 2.02-1.97 (m, 2H), 1.47-1.44 (m, 2H).

Step 2. To a solution of (1-methylsulfonyl-4-piperidyl) methanesulfonate (6.75 g, 26.2 mmol, 1.5 eq) and ethyl 3-hydroxy1H-pyrazole-5-carboxylate (2.73 g, 17.5 mmol, 1 eq) in DMF (50 mL) was added K₂CO₃ (7.25 g, 52.5 mmol, 3 eq). The mixture was stirred at 90° C. for 14 hours. On completion, the mixture was partitioned between ethyl acetate (20 mL*3) and water (100 mL), the combined organic phase was dried over anhydrous sodium sulfate, concentrated to give a residue. The residue was purified by column chromatography (SiO₂, Petroleum ether/Ethyl acetate=1:0 to 1:2) to give ethyl 3-[(1-methylsulfonyl-4-piperidyl)oxy]-1H-pyrazole-5-carboxylate (1.50 g, 4.73 mmol, 27% yield) as white solid. ¹H NMR (400 MHz, CDCl₃) δ=7.96 (s, 1H), 6.16 (s, 1H), 4.78-4.64 (m, 1H), 4.30 (q, J=7.2 Hz, 2H), 3.43-3.21 (m, 4H), 2.74 (s, 3H), 2.10-1.91 (m, 4H), 1.31 (t, J=7.2 Hz, 3H).

Step 3. A mixture of ethyl 3-[(1-methylsulfonyl-4-piperidyl)oxy]-1H-pyrazole-5-carboxylate (1.50 g, 4.73 mmol, 1 eq), 2-[tert-butyl(dimethyl)silyl]oxyethanol (833 mg, 4.73 mmol, 1 eq), PPh₃ (2.73 g, 10.4 mmol, 2.2 eq) in 2-MeTHF (15 mL) was degassed and purged with N₂ for 3 times, and then DIAD (2.39 g, 11.8 mmol, 2.5 eq) was added to the mixture dropwise at 0° C. and the mixture was stirred at 25° C. for 2 hours under N₂ atmosphere. On completion, the reaction mixture was concentrated to give a residue. The residue was purified by column chromatography (SiO₂, Petroleum ether/Ethyl acetate=1:0 to 2:1) to give ethyl 2-[|2-|tert-butyl(dimethyl)silyl|oxyethyll-5-1(1-methylsulfonyl-4-piperidyl)oxy]pyrazole-3-carboxylate (2.00 g, 4.20 mmol, 89% yield) as yellow oil. ¹H NMR (400 MHz, CDCl₃) δ=6.19 (s, 1H), 4.70 (quin, J=4.4 Hz, 1H), 4.56 (t, J=5.6 Hz, 2H), 4.32 (q, J=7.2 Hz, 2H), 3.89 (t, J=5.6 Hz, 2H), 3.43-3.27 (m, 4H), 2.80 (s, 3H), 2.05-2.02 (m, 4H), 1.36 (t, J=7.2 Hz, 3H), 0.81 (s, 9H), −0.04-0.09 (m, 6H).

Step 4. To a mixture of ethyl 2-[2-[tert-butyl(dimethl)silyl]oxyethyl]-5-[(1-methylsulfonyl-4-piperidyl)oxy]pyrazole-3- carboxylate (2.27 g, 4.77 mmol, 1 eq) in THE (20 mL) was added LiAlH₄ (181 mg, 4.77 mmol, 1 eq) at 0° C. The mixture was stirred at 0° C. for 1 hour under N₂ atmosphere. On completion, the reaction mixture was quenched with water (0.5 mL), 15% NaOH (0.5 mL) and water (0.5 mL), the mixture was concentrated to give [2-[2-[tert-butyl(dimethyl)silyl]oxyethyl]-5-[(1-methylsulfonyl-4-piperidyl) oxy]pyrazol-3-yl]methanol (1.00 g, 2.31 mmol, 48% yield) as a brown oil. LCMS: m/z 434.2 (M+1)

Step 5. To a solution of [2-[2-[tert-butyl(dimethyl)silyl]oxyethyl]-5-[(1-methylsulfonyl-4-piperidyl)oxy]pyrazol-3- yl]methanol (900 mg, 2.08 mmol, 1 eq) in ACN (10 mL) was added NIS (467 mg, 2.08 mmol, 1 eq). The mixture was stirred at 0° C. for 0.5 hour. On completion, the mixture was quenched with sat. Na₂SO₃ (20 mL) aqueous solution and extracted with ethyl acetate (25 mL*3), the combined organic phase was dried over anhydrous sodium sulfate, filtered and concentrated to give [2-[2-[tert-butyl(dimethyl)silyl]oxyethyl]-4-iodo-5-[(1-methylsulfonyl-4-piperidyl)oxy]pyrazol-3-yl]methanol (1.15 g, 2.06 mmol, 99% yield) as yellow oil. ¹H NMR (400 MHz, CDCl₃) δ=4.88-4.80 (m, 1H), 4.59 (s, 2H), 4.29-4.19 (m, 2H), 3.97-3.86 (m, 2H), 3.46-3.29 (m, 4H), 3.27-3.01 (m, 1H), 2.83 (s, 3H), 2.11-1.96 (m, 4H), 0.82 (s, 9H), 0.00 (s, 6H). LCMS: m/z 560.5 (M+1)

Step 6. To a solution of [2-[2-[tert-butyl(dimethyl)silyl]oxyethyl]-4-iodo-5-[(1-methylsulfonyl-4-piperidyl)oxy]pyrazol-3- yl]methanol (600 mg, 1.07 mmol, 1 eq) in DCM (5 mL) was added methylsulfonyl methanesulfonate (560 mg, 3.22 mmol, 3 eq) and TEA (326 mg, 3.22 mmol, 3 eq). The mixture was stirred at 25° C. for 1 hour. On completion, the mixture was concentrated to give [2-[2-[tert-butyl(dimethyl) silyl]oxyethyl]-4-iodo-5-[(1-methylsulfonyl-4-piperidyl)oxy]pyrazol-3-yl]methyl methanesulfonate (654 mg, 1.03 mmol, 96% yield) as brown oil. LCMS: m/z 638.0 (M+1)

I-175 was converted to Ex. 175 following procedures described in General Method M.

Ex. 176 was prepared following procedures described in General Method M and General Method I starting with the appropriate alcohol, tert-butyl (3R)-3-hydroxypiperidine-1-carboxylate, boronic ester G-1-1, and bromide H-4-1 at the appropriate steps.

Ex. 177 was prepared from 119-2 and 133-1 following procedures similar to those described in General Method H.

### Preparation of 2-[6-fluoro-1-tetrahydropyran-2-yl-5-(4,4,5,5-tetramethyl-1,3,2-dioxaborolan-2-yl) indazol-3-yl]ethynyl-triisopropyl-silane, 1-178

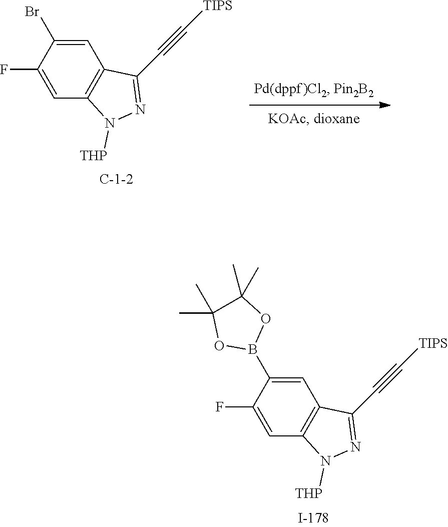

Step 1. A mixture of 2-(5-bromo-6-fluoro-1-tetrahydropyran-2-yl-indazol-3-yl) ethynyl-triisopropyl-silane (8 g, 16.7 mmol, 1 eq), Pin₂B₂ (6.36 g, 25.1 mmol, 1.5 eq), KOAc (4.91 g, 50.1 mmol, 3 eq) and cyclopentyl (diphenyl) phosphane;dichloromethane;dichloropalladium;iron (1.36 g, 1.67 mmol, 0.1 eq) in dioxane (80 mL) was degassed and purged with N₂ for 3 times, and then the mixture was stirred at 80° C. for 16 h under N₂ atmosphere. On completion, the reaction was concentrated in vacuum to give a residue. The residue was purified by column chromatography (SiO₂, Petroleum ether/EA=1:0 to 20:1) to give 2-[6-fluoro-1-tetrahydropyran-2-yl-5-(4,4,5,5-tetramethyl-1,3,2-dioxaborolan-2-yl) indazol-3-yl]ethynyl-triisopropyl-silane (5.50 g, 9.92 mmol, 59% yield, 95% purity) as a white solid. ¹H NMR (400 MHz, CDCl₃) δ=8.14 (d, J=2.4 Hz, 1H), 7.24-7.19 (m, 1H), 5.62 (d, J=2.8, 9.2 Hz, 1H), 4.08-4.00 (m, 1H), 3.79-3.66 (m, 1H), 2.60-2.38 (m, 1H), 2.15-2.08 (m, 1H), 1.78-1.71 (m, 2H), 1.69-1.65 (m, 1H), 1.42 (s, 3H), 1.38 (s, 13H), 1.20-1.18 (m, 18H).

Ex. 178 was prepared via procedures described Ex. 122 from the appropriate alcohol, tert-butyl (3S)-3-hydroxypiperidine-1-carboxylate, I-178, and I-156 in the appropriate steps.

Ex. 179 were prepared in a manner described in General Method P starting from appropriate alcohol, (3-isopropylisoxazol-5-yl)methanol.

### Preparation of Ex. 180

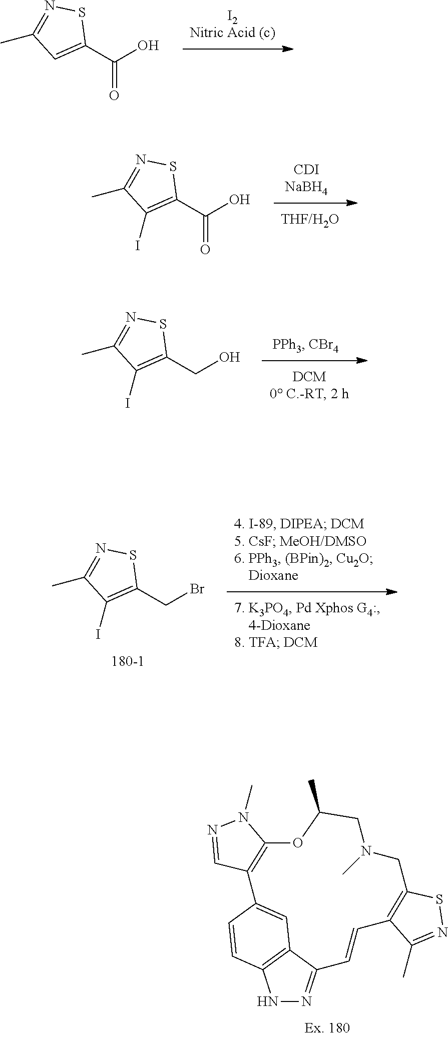

Step 1. To 3-methylisothiazole-5-carboxylic acid (438 mg, 3.06 mmol) was added Nitric Acid (275.41 mg, 3.06 mmol, 6 mL, 70% purity) and iodine (931.81 mg, 3.67 mmol). Heated to 100° C. for 3 hr. Cooled in an ice bath and carefully quenched with 1M sodium bisulfite (aq. 3 mL). Reaction was diluted with DCM and water (20 mL) and the layers were separated. The aqueous layer was extracted again with DCM (2×20 mL). The combined organic layer was washed with brine and dried over sodium sulfate. Filtrate was concentrated under reduced pressure and the residue crystallized with EA/Hex to afford 4-iodo-3-methyl-isothiazole-5-carboxylic acid (350 mg, 1.30 mmol, 43% yield). Mother liquor was purified by flash column chromatography (automated system, 12 g silica, 0-10% MeOH in DCM) provided 4-iodo-3-methyl-isothiazole-5-carboxylic acid (350 mg, 1.30 mmol, 43% yield). LCMS: m/z 270.0 (M+1)

Step 2. To 4-iodo-3-methyl-isothiazole-5-carboxylic acid (400 mg, 1.49 mmol) in THF (5 mL) was added CDT (494.17 mg, 3.05 mmol) and stirred for 2 h at RT. The mixture was added slowly to a solution of Sodium Borohydride (281.22 mg, 7.43 mmol, 261.84 L) in water (2.5 mL). Allowed to stir for 5 minutes and then quenched with concentrated HCl carefully until pH is approximately 2. Reaction was diluted with EA and water (10 mL) and the layers were separated. The aqueous layer was extracted again with EA (2×10 mL). The combined organic layer was washed with brine and dried over sodium sulfate. Aqueous layer was re-extracted with DCM (3×20 mL). Flash column chromatography (automated system, 12 g silica, 0-45% EA in Hexanes) provided (4-iodo-3-methyl-isothiazol-5-yl)methanol (176 mg, 0.690 mmol, 46% yield). LCMS: m/z 255.8 (M+1)

Step 3. To (4-iodo-3-methyl-isothiazol-5-yl)methanol (100 mg, 0.392 mmol) in DCM (2 mL) was added triphenylphosphine (206 mg, 0.784 mmol) followed by carbon tetrabromide (195 mg, 0.588 mmol). Stir at 0° C. for 18 hr as temperature increases to ambient. Quenched with water (1 mL) and worked up with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-30% EA in Hexanes) provided 5-(bromomethyl)-4-iodo-3-methyl-isothiazole, 180-1 (89 mg, 0.280 mmol, 71% yield). LCMS: m/z 317.7/319.7 (M/M+2) [01667]5-(bromomethyl)-4-iodo-3-methyl-isothiazole was combined with 89-2 and converted to Ex. 180 via methods described in General Method P.

Ex. 181 was prepared in a similar manner to those described in General Method Musing 116-1 and 147-1.

### Preparation of [2-[2-[tert-butyl (dimethyl)silyl]oxyethyl]-4-iodo-5-[(3S)-1-methylsulfonylpyrrolidin-3-yl]oxy-pyrazol-3-yl]methyl methanesulfonate (I-182)

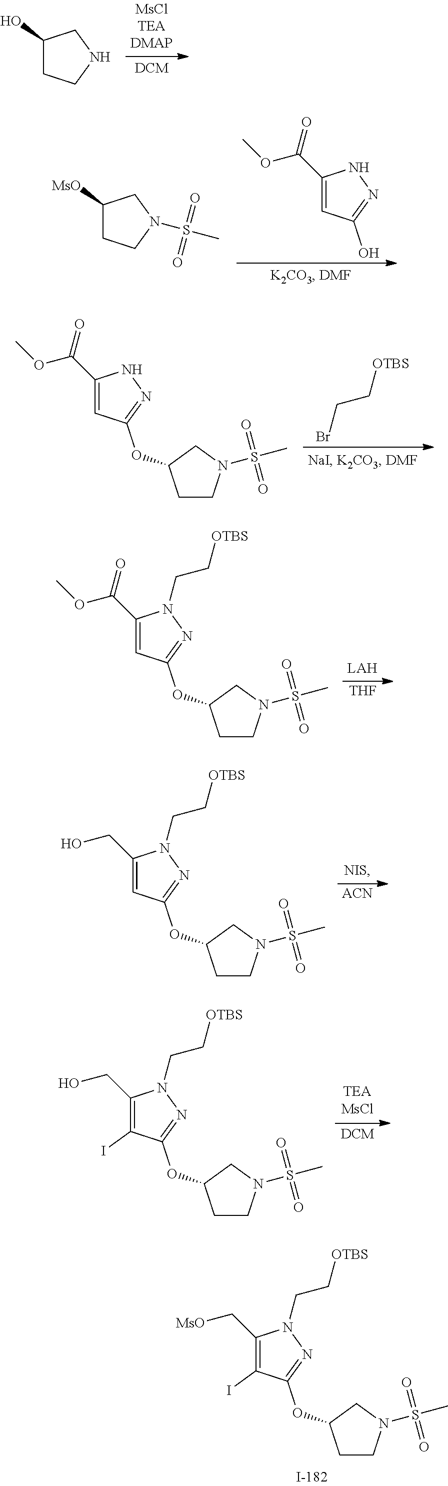

Step 1. To a mixture of (3R)-pyrrolidin-3-ol (10.0 g, 115 mmol, 1 eq) in DCM (100 mL) was added TEA (58.1 g, 574 mmol, 79.9 mL, 5 eq) and MsCl (52.6 g, 459 mmol, 4 eq), DMAP (1.40 g, 11.5 mmol, 0.1 eq) at 0° C. The mixture was stirred at 25° C. for 0.5 h. On completion, the mixture was quenched with dilute citric acid (300 mL). The mixture was separated and the aqueous layer was extracted with DCM (100 mL×3). The combined organic phase was dried over Na₂SO₄, filtered and the filtrate was concentrated to give to give [(3R)-1-methylsulfonylpyrrolidin-3-yl]methanesulfonate (20.5 g, crude) as a yellow solid.

Step 2. To a solution of [(3R)-1-methylsulfonylpyrrolidin-3-yl]methanesulfonate (1.40 g, 5.75 mmol, 1.1 eq), methyl 3-hydroxy-1H-pyrazole-5-carboxylate (743 mg, 5.23 mmol, 1 eq) in DMF (14 mL) was added K₂CO₃ (2.17 g, 15.7 mmol, 3 eq). The mixture was stirred at 60° C. for 16 h. On completion, the mixture was diluted with water (50 mL) and extracted with ethyl acetate (15 mL×3). The combined organic phase was washed with water (50 mL*2) and dried over Na₂SO₄, filtered and the filtrate was concentrated to give a residue. The residue was purified by column chromatography (SiO₂, PE:THF=1:1) to give methyl 3-[(3S)-1-methylsulfonylpyrrolidin-3-yl]oxy-1H-pyrazole-5-carboxylate (530 mg, crude) as a white solid. LCMS: m/z 290.0 (M+1)

Step 3. To a solution of methyl 3-[(3S)-1-methylsulfonylpyrrolidin-3-yl]oxy-1H-pyrazole-5-carboxylate (2.31 g, 7.98 mmol, 1 eq) in DMF (20 mL) was added NaI (1.20 g, 7.98 mmol, 1 eq), 2-bromoethoxy-tert-butyl-dimethyl-silane (2.87 g, 12.0 mmol, 1.5 eq) and K₂CO₃ (3.31 g, 24.0 mmol, 3 eq), then the mixture was stirred at 60° C. for 16 h. On completion, the mixture was diluted with water (60 mL) and extracted with ethyl acetate (20 ml×3). The combined organic phase was washed with water (60 mL*2) and dried over Na₂SO₄, filtered and the filtrate was concentrated to give a residue. The residue was purified by column chromatography (SiO₂, PE:THF=1:1) to give methyl 2-[2-[tert-butyl(dimethyl)silyl|oxyethyl|-5-|(3S)-1-methylsulfonylpyrrolidin-3-yl|oxy -pyrazole-3-carboxylate (2.04 g, crude) as a yellow oil. LCMS: m/z 448.6 (M+1)

Step 4. To a mixture of methyl 2-[2-[tert-butyl(dimethyl)silyl]oxyethyl]-5-[(3S)-1-methylsulfonylpyrrolidin-3-yl]oxy-pyrazole-3-carboxylate (2.14 g, 4.78 mmol, 1 eq) in THF (20 mL) was added LiAlH₄ (327 mg, 8.61 mmol, 1.8 eq) at 0° C. The mixture was stirred at 25° C. for 2 h. On completion, the mixture was quenched with water (0.3 ml) and 15% aq. NaOH (0.3 ml) drop by drop, then water (1 ml). The residue was purified by column chromatography (SiO₂, PE:THF=1:1) to give [2-[2-[tert-butyl (dimethyl)silyl]oxyethyl]-5-(1-methylsulfonylpyrrolidin-3-yl)oxy-pyrazol-3-yl]methanol (1.10 g, crude) as a white oil. LCMS: m/z 420.0 (M+1)

Step 5. To a solution of [2-[2-[tert-butyl(dimethyl)silyl]oxyethyl]-5-(1-methylsulfonyl pyrrolidin-3-yl)oxy-pyrazol-3-yl]methanol (1.05 g, 2.50 mmol, 1 eq) in ACN (10 mL) was added NIS (619 mg, 2.75 mmol, 1.1 eq) at 0° C. The mixture was stirred at 25° C. for 1 h. On completion, the mixture was quenched with sat. Na₂SO₃ (30 mL) at 0° C. and extracted with DCM (10 mL×3), the combined organic phase was dried over anhydrous Na₂SO₄, filtered and the filtrate was concentrated to give a residue. The residue was purified by column chromatography (SiO₂, PE:THF=2:1) to give [2-[2-[tert-butyl(dimethyl)silyl]oxyethyl]-4-iodo-5-[(3S)-1-methylsulfonylpyrrolidin-3-yl]oxy-pyrazol-3-yl]methanol (1.33 g, crude) as a white solid. LCMS: m/z 546.1 (M+1)

Step 6. To a solution of [2-[2-[tert-butyl(dimethyl)silyl]oxyethyl]-4-iodo-5-[(3S)-1-methylsulfonylpyrrolidin-3-yl]oxy-pyrazol-3-yl]methanol (1.06 g, 1.94 mmol, 1 eq) in DCM (10 mL) was added TEA (590 mg, 5.83 mmol, 3 eq), and then methylsulfonyl methanesulfonate (508 mg, 2.91 mmol, 1.5 eq) was added at 0° C. The mixture was stirred at 25° C. for 0.5 h. On completion, the mixture was quenched with dilute citric acid (30 mL). The mixture was separated and the aqueous layer was extracted with DCM (10 mL×3). The combined organic phase was dried over Na₂SO₄, filtered and the filtrate was concentrated to give a residue to give [2-[2-[tert-butyl (dimethyl)silyl]oxyethyl]-4-iodo-5-[(3S)-1-methylsulfonylpyrrolidin-3-yl]oxy-pyrazol-3-yl]methyl methanesulfonate, 1-181 (1.17 g, crude) as a yellow oil. LCMS: m/z 624.0 (M+1)

I-182 was combined with 89-2 and converted to Ex. 182 in a manner similar to those described in General Method M.

### Preparation of 5-(bromomethyl)-3-ethoxy-4-iodoisoxazole (I-183)

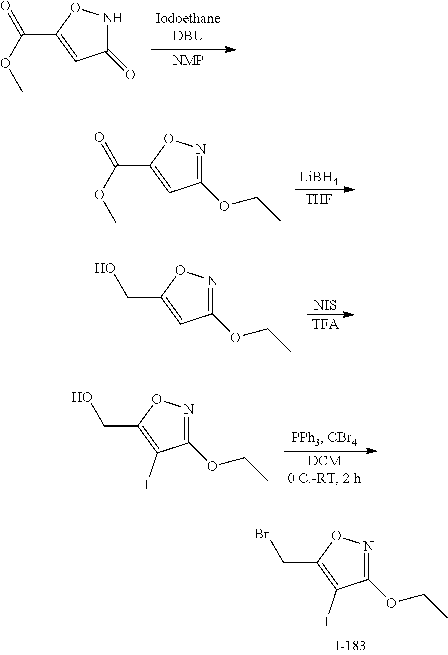

Step 1. To methyl 3-oxoisoxazole-5-carboxylate (1 g, 6.99 mmol) in NMP (21.37 mL) was added DBU (1.38 g, 9.08 mmol, 1.36 mL). Stir as argon is bubbled through and Iodoethane (1.09 g, 6.99 mmol, 561.82 μL) was added slowly at 0° C. Stirred as temperature is kept cold for the next 4 hours then allowed to slowly warm to RT overnight. The reaction was diluted with DCM (50 mL) and cooled. HCl (aq) (2M, 50 mL) was added slowly while stirring at 0° C. The layers were partition and the organic layer was concentrated. The residue was loaded onto a silica plug and eluted with 30% EA in Hexanes (500 mL). The filtrate was concentrated to give a light orange oil. Re-dissolved in DCM (20 mL) and washed with brine followed by sodium bicarbonate (sat.) then dried over sodium sulfate. After filtration and concentration, methyl 3-ethoxyisoxazole-5-carboxylate (1.04 g, 6.08 mmol, 87% yield) was obtained. LCMS: m/z 172.0 (M+1)

Step 2. To methyl 3-ethoxyisoxazole-5-carboxylate (500 mg, 2.92 mmol) in THF (12.96 mL) was added LiBH₄ (127 mg, 5.8 mmol) under argon at 0° C. Stir for 0.5 hr and reaction was diluted with DCM and water (30 mL) and the layers were separated. The aqueous layer was extracted again with DCM (2×20 mL). The combined organic layer was washed with brine and dried over sodium sulfate. Flash column chromatography (automated system, 12 g silica, 0-30% EA in Hexanes) provided (3-ethoxyisoxazol-5-yl)methanol (263 mg, 1.84 mmol, 63% yield). LCMS: m/z 144.1 (M+1)

Step 3. To (3-ethoxyisoxazol-5-yl)methanol (263 mg, 1.84 mmol) in TFA (4 mL) was added NIS (620.06 mg, 2.76 mmol). Stir at 70° C. for 0.5 hr. Reaction was diluted with DCM and water (20 mL) after cooling and the layers were separated. The aqueous layer was extracted again with DCM (2×10 mL). The combined organic layer was washed with brine and dried over sodium sulfate. Flash column chromatography (automated system with ELSD, 12 g silica, 0-30% EA in Hexanes) provided (3-ethoxy-4-iodo-isoxazol-5-yl)methanol (264 mg, 0.981 mmol, 53% yield). LCMS: m/z 269.9 (M+1)

Step 4. To (3-ethoxy-4-iodo-isoxazol-5-yl)methanol (130 mg, 0.483 mmol) in DCM (2.5 mL) was added Triphenylphosphine (253 mg, 0.966 mmol) followed by carbon tetrabromide (240 mg, 0.725 mmol) at 0° C. Stirred for 2 hr. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 0-30% EA in Hexanes) provided 5-(bromomethyl)-3-ethoxy-4-iodo-isoxazole (140 mg, 0.422 mmol, 87% yield). LCMS: m/z 331.7 (M+1)

I-183 was combined with 89-2 and converted to Ex. 183 following procedures similar to those described in General Method P.

### Preparation of 2-[(10S,17E)-16-bromo-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 184)

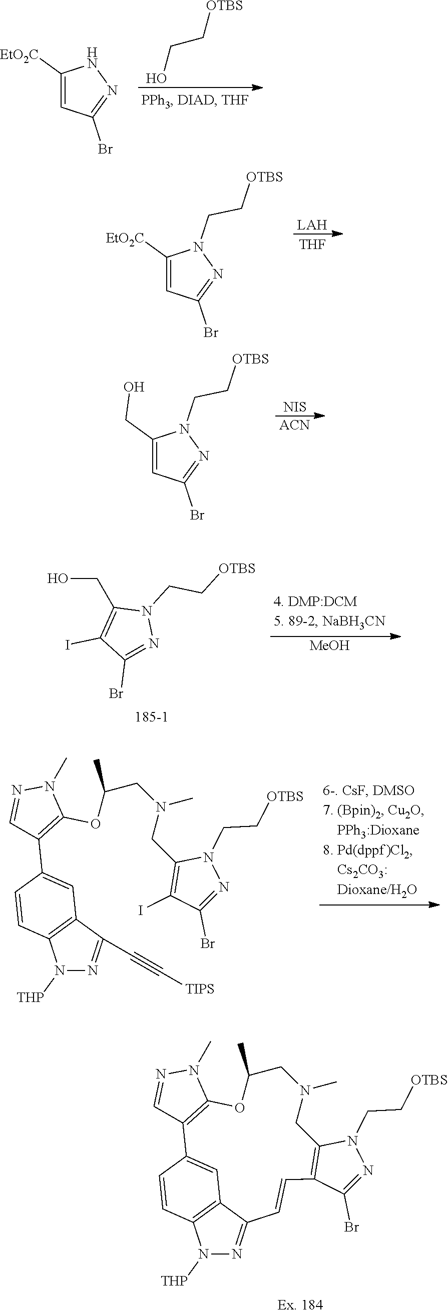

Step 1. To a solution of ethynyl(triisopropyl)silane (33.4 g, 183 mmol, 41 mL, 2.5 eq) in THF (200 mL) at −78° C. was added n-BuLi (2.5 M, 88 mL, 3 eq) under N₂. The mixture was stirred at the −78° C. for 1 hr, and then BF₃·Et₂O (26.0 g, 183 mmol, 23 mL, 2.5 eq) was added. After 0.2 hr, methyl 3-chloro-3-oxo-propanoate (10.0 g, 73.2 mmol, 7.82 mL, 1 eq) in THE (50 mL) was added. The mixture was stirred at −78° C. for 2 h. On completion, the mixture was quenched with sat. NH₄Cl (500 mL) and water (500 mL) and extracted with ethyl acetate (500 mL×3), the combined organic phase was dried over anhydrous sodium sulfate, filtered and concentrated to give methyl 3-oxo-5-triisopropylsilyl-pent-4-ynoate (60.0 g, crude) as an orange oil. The residue was purified by column chromatography (SiO₂, Petroleum ether/Ethyl acetate=1/0 to 10/1) to give methyl 3-oxo-5-triisopropylsilyl-pent-4-ynoate (9.96 g, 35.3 mmol, 17% yield) as a red oil. ¹H NMR (400 MHz, CDCl₃-d) δ=11.78-11.58 (m, 1H), 3.78-3.74 (m, 4H), 3.62-3.60 (m, 1H), 1.11-1.10 (m, 21H).

Step 2. To a solution of methyl 3-oxo-5-triisopropylsilyl-pent-4-ynoate (6.83 g, 24.2 mmol, 1 eq) in MeOH (70 mL) was added methylhydrazine (3.34 g, 29.0 mmol, 4 mL, 1.2 eq) at 0° C. The mixture was stirred at 25° C. for 16 hr. On completion, the mixture was filtered and concentrated to give a residue. The residue was purified by column chromatography (SiO₂, DCM:MeOH=1:0 to 10:1) to give 2-methyl-5-(2-triisopropylsilylethynyl)pyrazol-3-ol (3.15 g, 10.3 mmol, 43% yield, 91% purity) as a brown solid. ¹H NMR (400 MHz, DMSO-d₆) δ=11.46-11.02 (m, 1H), 5.48 (s, 1H), 3.50 (s, 3H), 1.07 (s, 21H); LCMS: m/z 279.2 (M+1).

Step 3. To a solution of 2-methyl-5-(2-triisopropylsilylethynyl)pyrazol-3-ol (1.00 g, 3.59 mmol, 1 eq) in dioxane (10 mL) was added 5-bromo-1-tetrahydropyran-2-yl-3-vinyl-indazole (1.21 g, 3.95 mmol, 1.1 eq) and [2-(2-aminophenyl)phenyl]-methylsulfonyloxy-palladium;ditert-butyl-[3,6-dimethoxy-2-(2,4,6-triisopropylphenyl)phenyl]phosphane (307 mg, 0.359 mmol, 0.1 eq) then the mixture was added K₂CO₃ (993 mg, 7.18 mmol, 2 eq) under N₂. The mixture was stirred at 100° C. for 3 hr. On completion, the mixture was filtered and concentrated to give a residue. The residue was purified by column chromatography (SiO₂, DCM/MeOH=1/0 to 10/1) to give 2-methyl-4-(1-tetrahydropyran-2-yl-3-vinyl-indazol-5-yl)-5-(2-triisopropylsilylethynyl)pyrazol-3-ol (960 mg, 1.77 mmol, 49% yield, 93% purity) as a brown solid. ¹H NMR (400 MHz, DMSO-d₆) δ=11.60-10.26 (m, 1H), 8.15 (s, 1H), 7.75 (s, 1H), 7.69 (s, 1H), 6.97 (dd, J=11.4, 18.0 Hz, 1H), 6.05 (d, J=18.0 Hz, 1H), 5.84 (dd, J=2.4, 9.6 Hz, 1H), 5.50 (d, J=12.4 Hz, 1H), 3.88 (br d, J=11.2 Hz, 1H), 3.79-3.71 (m, 1H), 3.63 (s, 3H), 2.45-2.31 (m, 2H), 2.08-2.00 (m, 1H), 1.96 (br dd, J=2.4, 13.1 Hz, 1H), 1.76 (td, J=3.2, 6.3 Hz, 2H), 1.59 (hr d, J =3.6 Hz, 3H), 1.05-1.01 (m, 18H); LCMS: m/z 505.2 (M+1).

Steps 4-5 were conducted similarly to Ex. 192

Steps 6-8 were conducted according to General Method H to afford Ex. 184.

### Preparation of 2-{(10S,17E)-16-[(azetidin-3-yl)oxy]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 185)

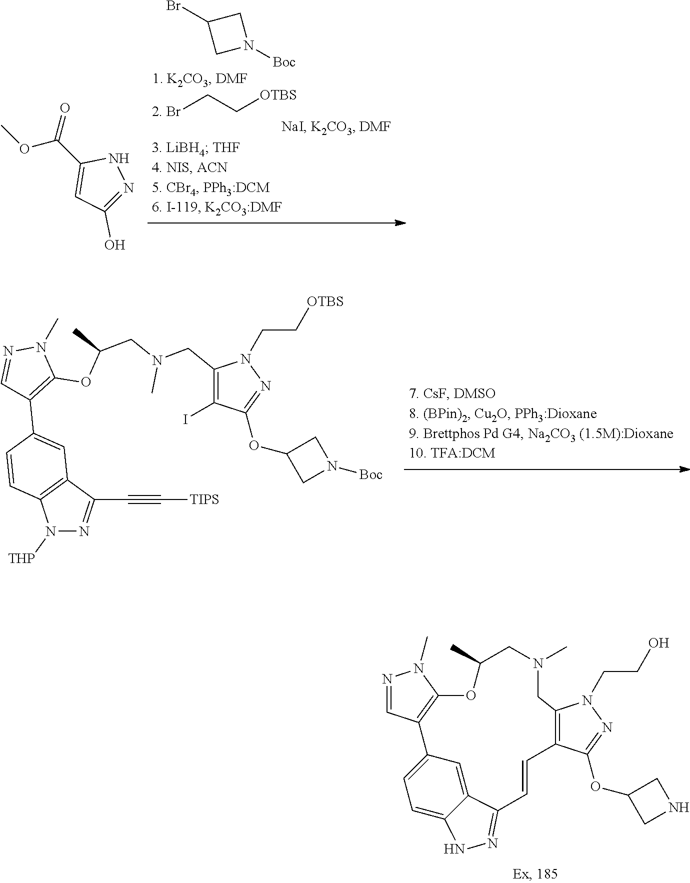

Steps 1-2 were conducted following procedures described in General Method 0.

Step 3. A mixture of methyl 5-(1-tert-butoxycarbonylazetidin-3-yl)oxy-2-[2-[tert-butyl(dimethyl)silyl]oxyethyl]pyrazole-3-carboxylate (3.70 g, 8.12 mmol, 1.00 eq) in THF (40 mL), added LiBH₄ (1.77 g, 81.2 mmol, 10.0 eq) at 0° C. and then the mixture was stirred at 25° C. for 16 h. On completion, the reaction mixture was quenched by addition NH₄Cl (80 mL) at 0° C. and extracted with EA (70 mL*3). The combined organic layers were dried over Na₂SO₄. The reaction mixture was concentrated under reduced pressure to remove solvent. The residue was purified by column chromatography (SiO₂, Petroleum ether/Ethyl acetate=1/0 to 0/1) to give tert-butyl 3-[1-[2-[tert-butyl(dimethyl)silyl]oxyethyl]-5-(hydroxymethyl)pyrazol-3-yl]oxyazetidine-1-carboxylate (2.60 g, 6.08 mmol, 75% yield) as a white solid. LCMS: m/z 428.3 (M+1)

Steps 4-5 were conducted following procedures described in General Method 0.

Steps 6-10 were conducted following procedures described in General Method Q to afford Ex. 185.

### Preparation of (10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol (Ex. 186)

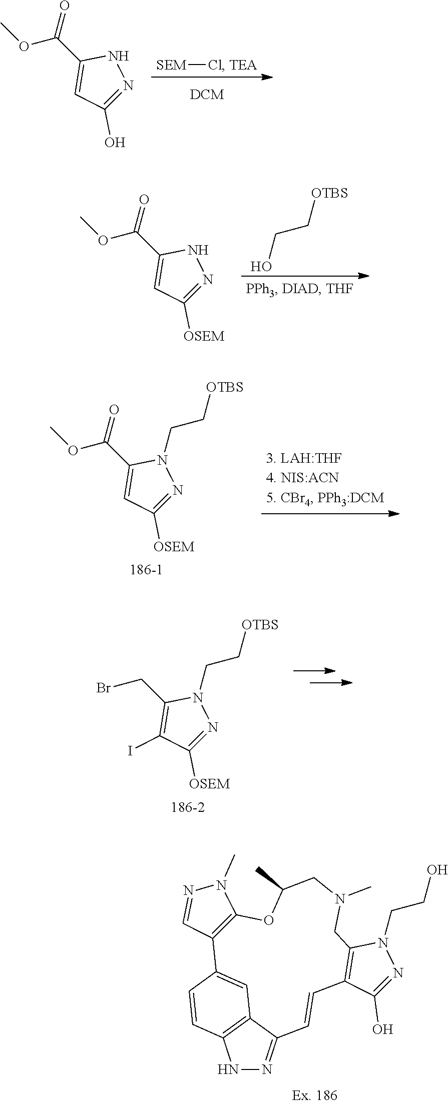

Step 1. To a mixture of methyl 3-hydroxy-1H-pyrazole-5-carboxylate (20.0 g, 140 mmol, 1.00 eq), TEA (85.0 g, 844 mmol, 117 mL, 6.00 eq) in DCM (200 mL) was added SEM-C1 (70.3 g, 422 mmol, 3.00 eq) at 0° C. and then the mixture was stirred at 25° C. for 2 h. On completion, the mixture was quenched with water (200 mL) and extracted with DCM (55 mL×3), the combined organic phase was dried over anhydrous sodium sulfate, filtered and concentrated to give a residue. The residue was purified by column chromatography (SiO₂, Petroleum ether/Ethyl acetate=1:0 to 0:1) to give methyl 3-(2-trimethylsilylethoxymethoxy)-1H-pyrazole-5-carboxylate (11.1 g, 40.9 mmol, 29% yield) as pink solid. ¹H NMR (400 MHz, CDCl₃) δ=6.32-6.30 (m, 1H), 5.28-5.25 (m, 2H), 3.88-3.86 (m, 3H), 3.78-3.73 (m, 2H), 1.25-1.18 (m, 2H), 0.96-0.92 (m, 2H), −0.03-0.04 (m, 9H). LCMS: m/z 273.2 (M+1)

Step 2. A mixture of methyl 3-(2-trimethylsilylethoxymethoxy)-1H-pyrazole-5-carboxylate (10.0 g, 36.7 mmol, 1.00 eq), 2-[tert-butyl(dimethyl)silyl]oxyethanol (14.2 g, 80.7 mmol, 2.20 eq), PPh₃ (21.1 g, 80.7 mmol, 2.20 eq) in THE (100 mL) was degassed and purged with N₂ for 3 times, and then DIAD (16.3 g, 80.7 mmol, 15.7 mL, 2.20 eq) was added at 0° C., the mixture was stirred at 25° C. for 2 h under N₂ atmosphere. On completion, the mixture was quenched with water (150 mL) and extracted with ethyl acetate (100 mL×3), the combined organic phase was dried over anhydrous sodium sulfate, filtered and concentrated to give a residue. The residue was purified by column chromatography (SiO₂, Petroleum ether/Ethyl acetate=1:0 to 4:1) to give methyl 2-[2-[tert-butyl(dimethyl)silyl]oxyethyl]-5-(2-trimethylsilylethoxymethoxy)pyrazole-3-carboxylate, 186-1 (10.0 g, 23.2 mmol, 63.0% yield) as yellow oil. LCMS: m/z 431.6 (M+1) [01697]186-1 was converted to Ex. 186 in a manner similar to those described in the synthesis of Ex. 148.

### Preparation of (10S,17E)-16-{I3-(methanesulfonyl)cyclobutyl|oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine (Ex. 187)

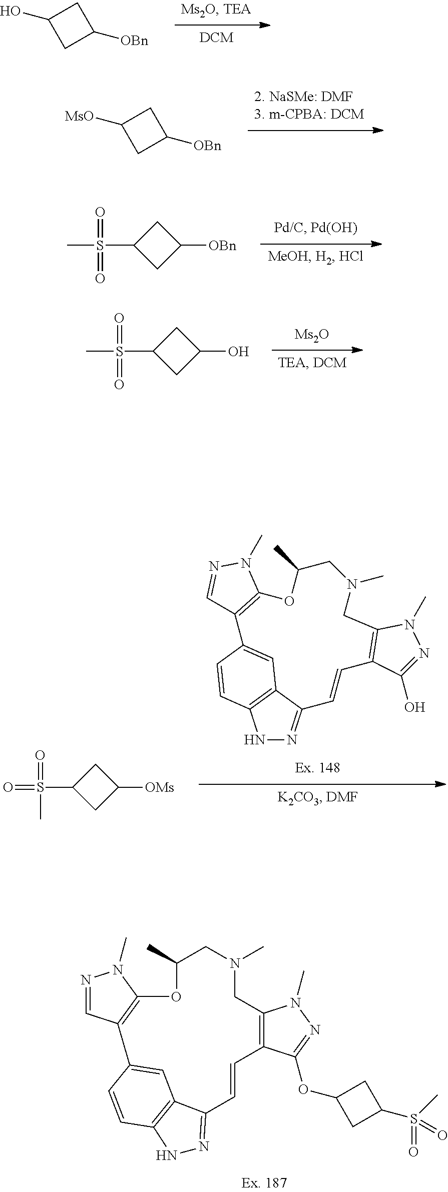

Step 1. To a solution of 3-benzyloxycyclobutanol (1.00 g, 5.61 mmol, 1 eq) and TEA (1.70 g, 16.8 mmol, 2 mL, 3 eq) in DCM (10 mL) was added methylsulfonyl methanesulfonate (1.47 g, 8.42 mmol, 1.5 eq) at 0° C. The mixture was stirred at 25° C. for 2 h. On completion, the mixture was quenched with water (20 mL) and extracted with DCM (25 mL×3), the combined organic phase was dried over anhydrous sodium sulfate, filtered and concentrated to give a residue. The residue was purified by column chromatography (SiO₂, DCM/MeOH=10:0 to 0:1) to give (3-benzyloxycyclobutyl) methanesulfonate (1.30 g, 5.07 mmol, 90% yield) as a yellow liquid. ¹H NMR (400 MHz, CDCl₃) δ=7.39-7.30 (m, 5H), 4.72-4.60 (m, 1H), 4.48-4.40 (m, 2H), 3.81-3.68 (m, 1H), 3.02-2.96 (m, 3H), 2.89-2.79 (m, 2H), 2.40-2.29 (m, 2H).

Steps 2-3 were conducted following procedures similar to those described in the synthesis of I-139.

Step 4. A mixture of (3-methylsulfonylcyclobutoxy)methylbenzene (100 mg, 0.416 mmol, 1 eq), Pd/C (20 mg, 0.416 mmol, 10% purity, 1 eq) and Pd(OH)₂ (20 mg, 0.014 mmol, 10% purity, 2 eq) in MeOH (1 mL) was degassed and purged with H₂ for 3 times, and then the mixture was stirred at 25° C. for 16 h under H₂ atmosphere at 50 Psi. On completion, the mixture was filtrated to remove Pd/C and Pd(OH)₂ to give 3-(methylsulfonyl)cyclobutan-1-ol (60 mg crude) as a white solid.

Step 5. To a solution of 3-methylsulfonylcyclobutanol (60 mg, 0.399 mmol, 1 eq) and TEA (121 mg, 1.20 mmol, 3 eq) in DCM (3 mL) was added methylsulfonyl methanesulfonate (139 mg, 0.799 mmol, 2 eq) at 0° C. The mixture was stirred at 25° C. for 2 h. On completion, the mixture was extracted with water (10 mL) and DCM (10 mL×3), the combined organic phase was dried over anhydrous sodium sulfate, filtered and concentrated to give a residue. The residue was purified by column chromatography (SiO₂, PE/THF=10:0 to 0:1) to give (3-methylsulfonylcyclobutyl) methanesulfonate (87 mg, 0.381 mmol, 95% yield) as a white solid. ¹H NMR (400 MHz, CDCl3-d) δ=5.18 (m, 1H), 3.76-3.66 (m, 1H), 2.96 (s, 3H), 2.92 (td, J=4.0, 7.6 Hz, 2H), 2.82-2.80 (m, 3H), 2.78-2.70 (m, 2H).

Step 6. (3-methylsulfonylcyclobutyl) methanesulfonate was combined with Ex. 148 in a manner similar to those described in General Method R to afford Ex. 187

### Preparation of (2R)-2-chloro-N-[(3-iodo-1,5-dimethyl-pyrazol-4-yl)methyl]-N-isopropyl-propan -1-amine, I-188

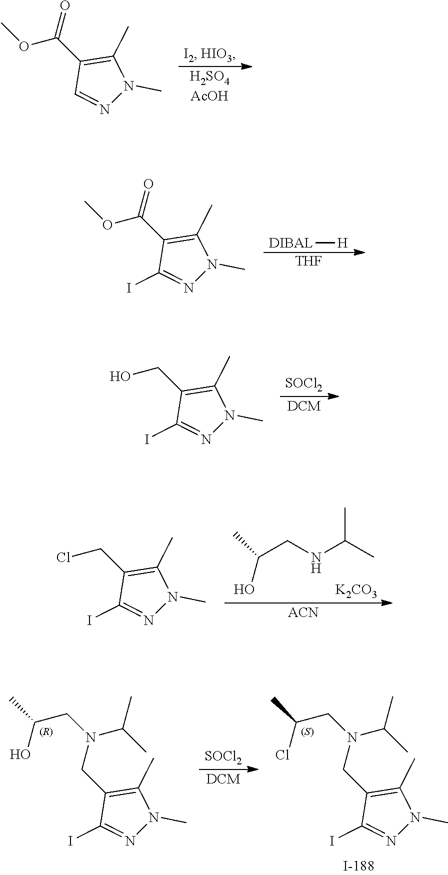

Step 1. A mixture of methyl 1,5-dimethylpyrazole-4-carboxylate (3.50 g, 22.7 mmol, 1 eq), 12 (4.61 g, 18.1 mmol, 0.8 eq) and iodic acid (1.88 g, 10.6 mmol, 0.47 eq) in H₂SO₄ (9.4 mL) and AcOH (26 mL) was stirred at 90° C. for 5 h. On completion, the mixture was treated with AcONa (5.60 g), and the AcOH was vacuum-distilled. The residue was dissolved in EA (50 mL), washed in succession with sat. NaHCO₃ (50 mL*2) and sat·Na₂SO₃ (50 mL*2) solutions, and dried over Na₂SO₄, filtered and concentrated to give a residue. The residue was purified by column chromatography (SiO₂, PE:THF=1:0 to 3:1) to give methyl 3-iodo-1,5-dimethyl-pyrazole-4-carboxylate (1.75 g, 6.25 mmol, 27% yield) as a white solid. LCMS: m/z 280.9 (M+1)

Step 2. A solution of methyl 3-iodo-1,5-dimethyl-pyrazole-4-carboxylate (1.75 g, 6.25 mmol, 1 eq) in THF (20 mL) was degassed and purged with N₂ for 3 times, and then DIBAL-H (1 M, 15.6 mL, 2.5 eq) was added dropwise at 0° C. The mixture was stirred at 25° C. for 2 h under N₂ atmosphere. On completion, the mixture was quenched with water (0.6 mL), 15% NaOH (0.6 mL), and water (1.5 mL), then the mixture was dried over anhydrous sodium sulfate, filtered and concentrated to give a residue. The residue was purified by column chromatography (SiO₂, PE:THF=1:0 to 2:1) to give (3-iodo-1,5-dimethyl-pyrazol-4-yl)methanol (1.57 g, 6.23 mmol, 100% yield) as a white solid. LCMS: m/z 252.9 (M+1)

Step 3. To a solution of (3-iodo-1,5-dimethyl-pyrazol-4-yl)methanol (1.34 g, 5.33 mmol, 1 eq) in DCM (14 mL) was added SOCl₂ (1.90 g, 15.9 mmol, 3 eq) at 0° C. The mixture was stirred at 25° C. for 0.5 h. On completion, the mixture was concentrated to give 4-(chloromethyl)-3-iodo-1,5-dimethyl-pyrazole (1.44 g, 5.32 mmol, 100% yield) as a white solid.

Step 4. To a mixture of 4-(chloromethyl)-3-iodo-1,5-dimethyl-pyrazole (1.44 g, 5.32 mmol, 1 eq) and (2R)-1-(isopropylamino)propan-2-ol (686 mg, 5.86 mmol, 1.1 eq) in ACN (15 mL) was added K₂CO₃ (2.21 g, 15.9 mmol, 3 eq). The mixture was stirred at 40° C. for 1 h. On completion, the mixture was filtered and concentrated to give a residue. The residue was purified by column chromatography (SiO₂, Dichloromethane: Methanol=1:0 to 15:1) to give (2R)-1-[(3-iodo-1,5-dimethyl-pyrazol-4-yl)methyl-isopropyl-amino]propan-2-ol (1.5 g, 4.27 mmol, 80% yield) as colorless oil. LCMS: m/z 352.2 (M+1)

Step 5. To a solution of (2R)-1-[(3-iodo-1,5-dimethyl-pyrazol-4-yl)methyl-isopropyl-amino]propan-2-ol (1 g, 2.85 mmol, 1 eq) in DCM (10 mL) was added TEA (864 mg, 8.54 mmol, 1.19 mL, 3 eq) and SOCl₂ (406 mg, 3.42 mmol, 248 μL, 1.2 eq). The mixture was stirred at 25° C. for 0.5 h. On completion, the mixture was concentrated to give (2R)-2-chloro-N-[(3-iodo-1,5-dimethyl-pyrazol-4-yl)methyl]-N-isopropyl-propan -1-amine, I-188 (950 mg, 2.57 mmol, 90% yield) as a yellow solid. LCMS: m/z 370.0 (M+1)

### Preparation of (10R,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine (Ex. 188)

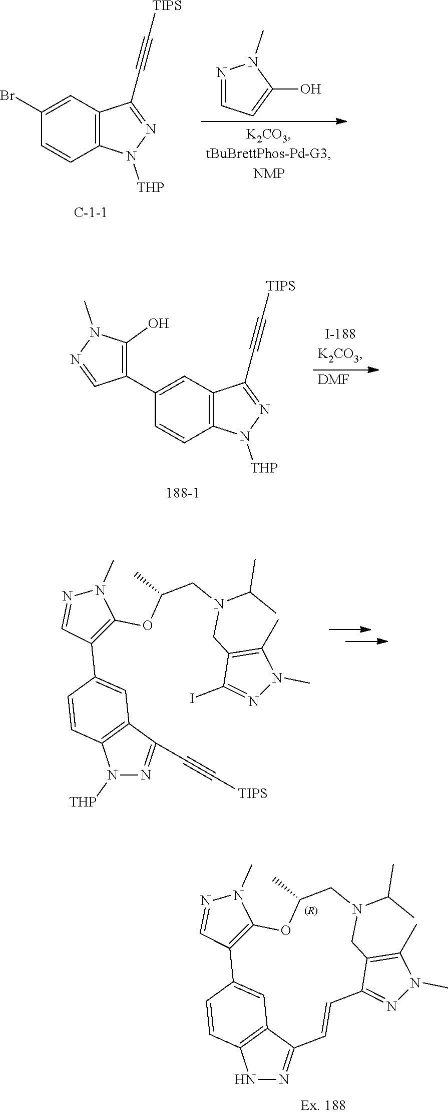

Step 1. To a solution of 2-(5-bromo-1-tetrahydropyran-2-yl-indazol-3-yl)ethynyl-triisopropyl-silane (5.00 g, 10.8 mmol, 1 eq) in NMP (120 mL) was added 2-methylpyrazol-3-ol (1.28 g, 13.0 mmol, 1.2 eq) and K₂CO₃ (2.99 g, 21.6 mmol, 2 eq), then [2-(2-aminophenyl)phenyl]-methylsulfonyloxy-palladium;ditert-butyl-[3,6-dimethoxy-2-(2,4,6-triisopropylphenyl)phenyl]phosphane (370 mg, 0.433 mmol, 0.04 eq) was added. The mixture was stirred at 130° C. for 2 h under N₂ atmosphere. On completion, the mixture was quenched by adding water (400 mL) and then filtered to produce a reddish brown solid. The residue was purified by column chromatography (SiO₂, DCM: THF=2:1 to 2:1) to give 2-methyl-4-[1-tetrahydropyran-2-yl-3-(2-triisopropylsilylethynyl) indazol-5-yl]pyrazol-3-ol (2.71 g, 5.66 mmol, 52% yield) as a brown solid. ¹H NMR (400 MHz, DMSO-d₆) δ=7.76 (br s, 4H), 5.92-5.81 (m, 1H), 3.90 (br d, J=11.6 Hz, 1H), 3.82-3.70 (m, 1H), 3.67-3.54 (m, 2H), 2.70 (s, 3H), 2.42-2.30 (m, 1H), 2.06-1.95 (m, 2H), 1.80-1.70 (m, 1H), 1.22-1.11 (m, 21H). LCMS: m/z 479.2 (M+1)

Step 2. To a mixture of 2-methyl-4-[1-tetrahydropyran-2-yl-3-(2-triisopropylsilylethynyl) indazol-5-yl]pyrazol-3-ol (1.48 g, 3.08 mmol, 1.2 eq) and (2R)-2-chloro-N-[(3-iodo-1,5-dimethyl-pyrazol-4-yl)methyl]-N-isopropyl-propan-1-amine (950 mg, 2.57 mmol, 1 eq) in DMF (10 mL) was added K₂CO₃ (1.07 g, 7.71 mmol, 3 eq). The mixture was stirred at 60° C. for 12 h. On completion, the mixture was quenched with water (20 mL) and extracted with ethyl acetate (15 mL×3), the combined organic phase was dried over anhydrous sodium sulfate, filtered and concentrated to give (2S)—N-[(3-iodo-1,5-dimethyl-pyrazol-4-yl)methyl]-N—isopropyl-2-[2-methyl-4-[l-tetrahydropyran-2-yl-3-(2-triisopropylsilylethynyl) indazol-5-yl]pyrazol-3-yl]oxy-propan-1-amine (2.45 g, crude) as a brown oil. LCMS: m/z 812.7 (M+1) [01713](2S)—N-[(3-iodo-1,5-dimethyl-pyrazol-4-yl)methyl]-N—isopropyl-2-[2-methyl-4-[1-tetrahydropyran-2-yl-3-(2-triisopropylsilylethynyl) indazol-5-yl]pyrazol-3-yl]oxy-propan-1-amine was converted to Ex. 188 following procedures described in the synthesis of General Method Q.

### Preparation of (10S,19E)-18-ethoxy-8,16-dimethyl-2,8,11,12,15,16-hexahydro-3,5-etheno-10,14-methanotripyrazolo[3,4-h:3′,4′-1:4″,3″-p][1,6]oxazacycloheptadecin-13(10H)-one (Ex. 189) and (10R,19E)-18-ethoxy-8,16-dimethyl-2,8,11,12,15,16-hexahydro-3,5-etheno-10,14-methanotripyrazolo[3,4-h:3′,4′-1:4″,3″-p][1,6]oxazacycloheptadecin-13(10H)-one (Ex. 190)

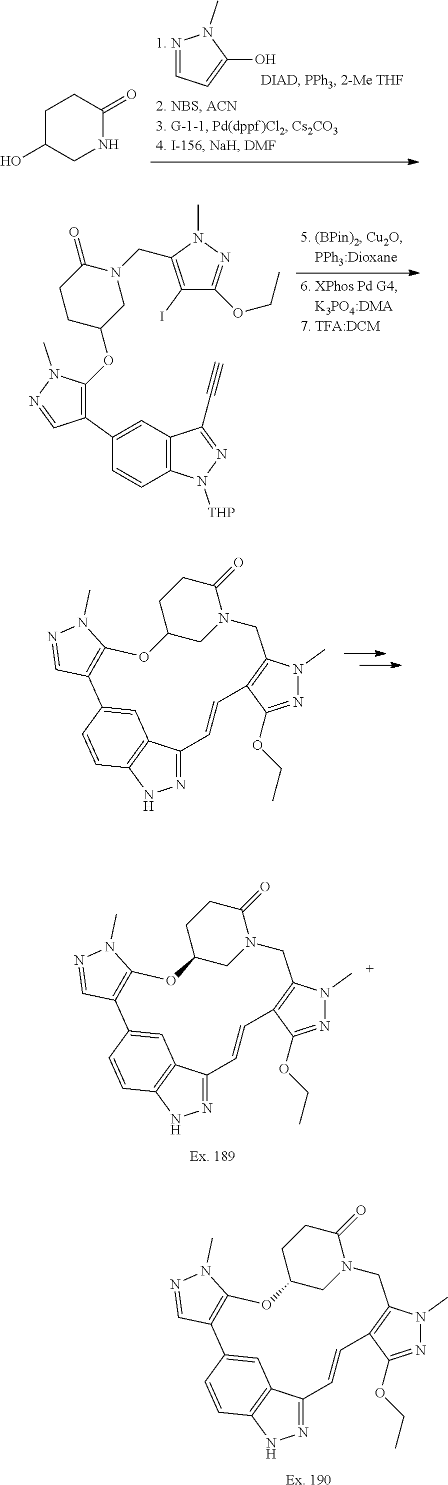

Steps 1-4 were conducted in a manner similar to those described in Ex. 122.

Steps 5-7 were conducted in a manner similar to those described in General Method L to afford a racemate.

Purification by SFC (column: DAICEL CHIRALCEL OX (250 mm*30 mm, 10 um);mobile phase: [C02-ACN/i-PrOH (0.1% NH₃H₂O)]; B %:65%, isocratic elution mode) afforded the arbitrarily assigned isomers Ex. 189 and Ex 190.

### Preparation of 2-[(10R,19E)-18-ethoxy-22-fluoro-8-methyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-o (Ex. 191)

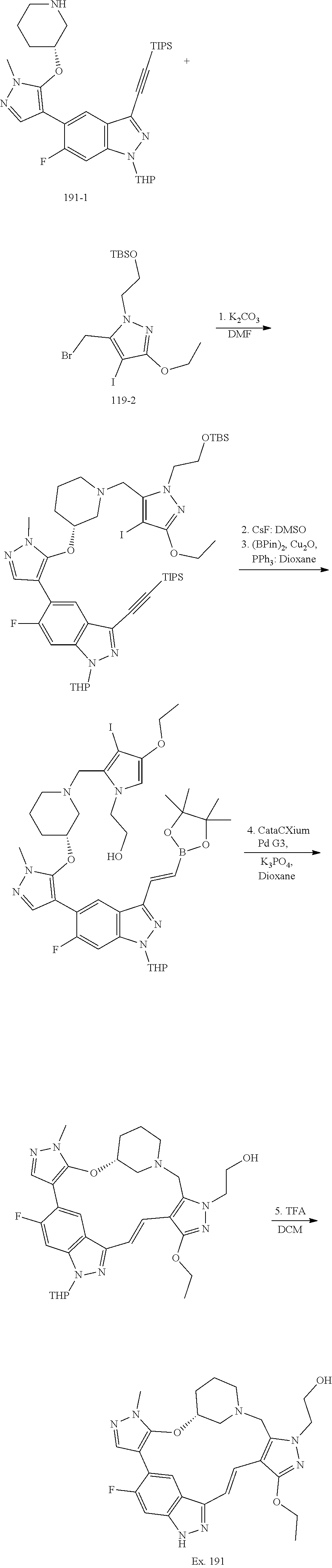

191-1, tert-butyl-[2-[3-ethoxy-5-[[(3R)-3-[4-[6-fluoro-1-tetrahydropyran-2-yl-3-(2-triisopropylsilylethynyl) indazol-5-yl]-2-methyl-pyrazol-3-yl]oxy-1-piperidyl]methyl]-4-iodo-pyrazol-1-yl]ethoxy]-dimethyl-silane, was prepared similarly to Ex. 176, following procedures described in General Method M and General Method I starting with the appropriate alcohol, tert-butyl (3R)-3-hydroxypiperidine-1-carboxylate and boronic ester I-178.

Step 1. To a solution of 2-[5-(bromomethyl)-3-ethoxy-4-iodo-pyrazol-1-yl]ethoxy-tert-butyl-dimethyl-silane (1.52 g, 3.1 mmol, 1.2 eq) and 2-[6-fluoro-5-[1-methyl-5-[[(3R)-3-piperidyl]oxy]pyrazol-4-yl]-1-tetrahydropyran-2-yl-indazol-3-yl]ethynyl-triisopropyl-silane (1.5 g, 2.59 mmol, 1 eq) in DMF (10 mL) was added K₂CO₃ (1.07 g, 7.76 mmol, 3 eq). The mixture was stirred at 80° C. for 2 hr. On completion, the mixture was diluted with water (50 mL) and extracted with ethyl acetate (30 mL*3). The combined organic layers were washed with brine (30 mL*3), dried over Na₂SO₄, filtered and concentrated to give tert-butyl-[2-[3-ethoxy-5-[[(3R)-3-[4-[6-fluoro-1-tetrahydropyran-2-yl-3-(2-triisopropylsilylethynyl) indazol-5-yl]-2-methyl-pyrazol-3-yl]oxy-1-piperidyl]methyl]-4-iodo-pyrazol-1-yl]ethoxy]-dimethyl-silane (2.25 g, crude) as a red oil. LCMS: m/z 988.5

Steps 2-3 were performed according to those described in General Method M.

Step 4. A mixture of 2-[3-ethoxy-5-[[(3R)-3-[4-[6-fluoro-1-tetrahydropyran-2-yl-3-[(E)-2-(4,4,5,5-tetramethyl-1,3,2-dioxaborolan-2-yl)vinyl]indazol-5-yl]-2-methyl-pyrazol-3-yl]oxy-1-piperidyl]methyl]-4-iodo-pyrazol-1-yl]ethanol (95 mg, 0.1H₂ mmol, 1 eq), CataCXium A Pd G3 (8.18 mg, 0.011 mmol, 0.1 eq), K3PO4 (71.5 mg, 0.337 mmol, 3 eq) in dioxane (1 mL) was degassed and purged with N₂ for 3 times, and then the mixture was stirred at 90° C. for 2 h under N₂ atmosphere. On completion, the mixture was filtered and concentrated under reduced pressure to give a residue. The residue was purified by column chromatography (SiO2, DCM/MeOH=0/20 to 1/20) to give 2-[(8R,19E)-17-ethoxy-26-fluoro-5-methyl-23-tetrahydropyran-2-yl-7-oxa-4,5,12,15,16,22,23-heptazahexacyclo[19.5.2.18,12.02,6.014,18.024,28]nonacosa-1(27),2(6),3,14(18),16,19,21,24(28),25-nonaen-15-yl]ethanol (28 mg, 0.047 mmol, 42% yield) as a red oil. LCMS: m/z 592.3 (M+1)

Step 5 was conducted via General Method M to afford Ex. 191.

### General Method S. Preparation of 2-{(11S,17E)-8,11-dimethyl-12-(propan-2-yl)-16-[(propan-2-yl)oxy]-2,8,10,11,12,13-hexahydro-14H-3,5-ethenotripyrazolo [3,4-f:3′,4′-j:4″,3″-n][1,4]oxazacyclopentadecin-14-yl}ethan-1-ol (Ex. 192) and 2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 193)

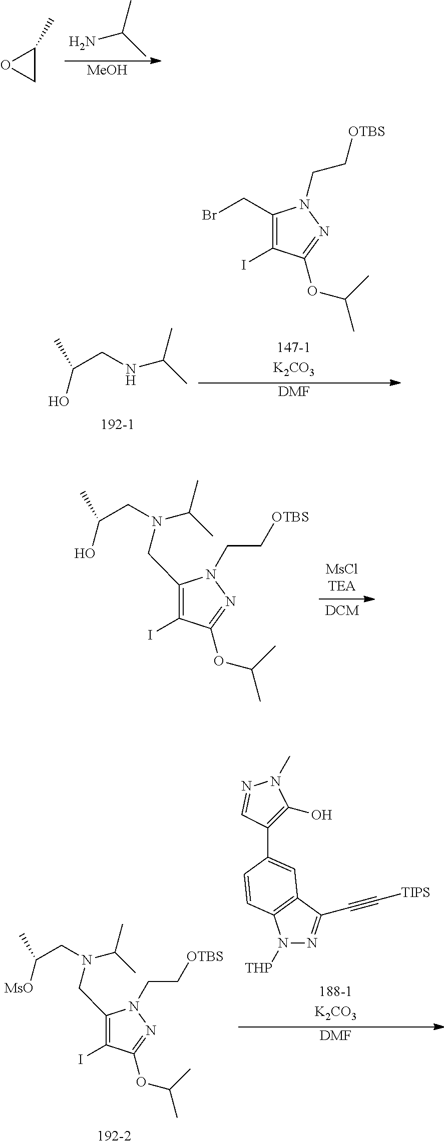

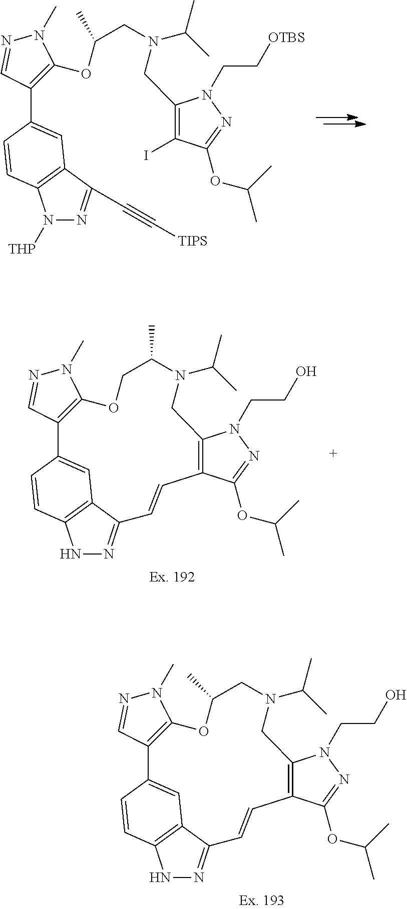

Step 1. To a solution of (2R)-2-methyloxirane (5.00 g, 86.0 mmol, 1 eq) and propan-2-amine (15.2 g, 258 mmol, 3 eq) in MeOH (80 mL). The mixture was stirred at 25° C. for 14 hours. On completion, the mixture was concentrated to give (2R)-1-(isopropylamino) propan-2-ol (8.60 g, 73.3 mmol, 85% yield) as yellow oil. ¹H NMR (400 MHz, CDCl₃) δ=3.76-3.66 (m, 1H), 2.85-2.69 (m, 2H), 2.40-2.27 (m, 1H), 2.19-1.94 (m, 1H), 1.15 (d, J=6.4 Hz, 3H), 1.09-1.04 (m, 6H).

Step 2. To a solution of (2R)-1-(isopropylamino) propan-2-ol (325 mg, 2.78 mmol, 1 eq) and 2-[5-(bromomethyl)-4-iodo-3-isopropoxy-pyrazol-1-yl]ethoxy-tert-butyl-dimethyl-silane, 147-1 (1.40 g, 2.78 mmol, 1 eq, in DMF (20 mL) was added K₂CO₃ (1.15 g, 8.35 mmol, 3 eq). The mixture was stirred at 25° C. for 14 hours. On completion, the mixture was quenched with water (20 mL) and extracted with ethyl acetate (25 mL×3), the combined organic phase was dried over anhydrous sodium sulfate, filtered and concentrated to give (2R)-1-[[2-[2-[tert-butyl(dimethyl) silyl]oxyethyl]-4-iodo-5-isopropoxy-pyrazol-3-yl]methyl-isopropyl-amino]propan-2-ol (1.50 g, 2.78 mmol, 99% yield) as colorless oil. ¹H NMR (400 MHz, CDCl₃) δ=4.87-4.74 (m, 1H), 4.31-4.09 (m, 2H), 3.93-3.82 (m, 2H), 3.80-3.56 (m, 3H), 2.88 (s, 2H), 2.40-2.23 (m, 2H), 1.37-1.32 (m, 6H), 1.08 (d, J=6.4 Hz, 6H), 1.04 (d, J=6.4 Hz, 3H), 0.81 (s, 9H), −0.04-0.12 (m, 6H). LCMS: m/z 540.1 (M+1)

Step 3. To a solution of (2R)-1-[[2-[2-[tert-butyl(dimethyl) silyl]oxyethyl]-4-iodo-5-isopropoxy-pyrazol-3-yl]methyl-isopropyl-amino]propan-2-ol (1.40 g, 2.59 mmol, 1 eq) in DCM (20 mL) was added methylsulfonyl methanesulfonate (1.36 g, 7.78 mmol, 3 eq) and TEA (656 mg, 6.49 mmol, 2.5 eq). The mixture was stirred at 25° C. for 0.5 hours. On completion, the mixture was quenched with water (10 mL) and extracted with DCM (5 mL×3), the combined organic phase was dried over anhydrous sodium sulfate, filtered and concentrated to give [(1R)-2-[[2-[2-[tert-butyl(dimethyl) silyl]oxyethyl]-4-iodo-5-isopropoxy-pyrazol-3-yl]methyl-isopropyl-amino]-1-methyl-ethyl]methanesulfonate (1.60 g, 2.59 mmol, 99% yield) as yellow oil. LCMS: m/z 618.8 (M+1)

Step 4. To a solution of [(1R)-2-[[2-[2-[tert-butyl(dimethyl) silyl]oxyethyl]-4-iodo-5-isopropoxy-pyrazol-3-yl]methyl-isopropyl-amino]-1-methyl-ethyl]methanesulfonate (1.60 g, 2.59 mmol, 1 eq) and 2-methyl-4-[1-tetrahydropyran-2-yl-3-(2-triisopropylsilylethynyl) indazol-5-yl]pyrazol-3-ol (1.24 g, 2.59 mmol, 1 eq) in DMF (30 mL) was added K₂CO₃ (1.07 g, 7.77 mmol, 3 eq). The mixture was stirred at 80° C. for 0.5 hours. On completion, the mixture was quenched with water (20 mL) and extracted with ethyl acetate (25 mL×3), the combined organic phase was dried over anhydrous sodium sulfate, filtered and concentrated to give (2R)—N-((1-(2-((tert-butyldimethylsilyl)oxy)ethyl)-4-iodo-3-isopropoxy-1H-pyrazol-5-yl)methyl)-N-isopropyl-2-((1-methyl-4-(1-(tetrahydro-2H-pyran-2-yl)-3-((triisopropylsilyl)ethynyl)-1H-indazol-5-yl)-1H-pyrazol-5-yl)oxy)propan-1-amine (2.50 g, 2.50 mmol, 96% yield) as yellow oil. LCMS: m/z 1000.4 (M+1)

Intermediate was taken forward following procedures described in General Method L Upon purification by prep-HPLC (column: Phenomenex luna C18 150*25 mm* 10 um; mobile phase: [water(FA)-ACN]; gradient:14%-44% B over 10 min), 2 isomers were isolated and determined to be inverted General Method S from step 4 and unexpected double inversion by-product, Ex. 193.

### Preparation of 4-[6-fluoro-1-tetrahydropyran-2-yl-3-(2-triisopropylsilylethynyl) indazol-5-yl]-2-methyl-pyrazol-3-ol (I-194)

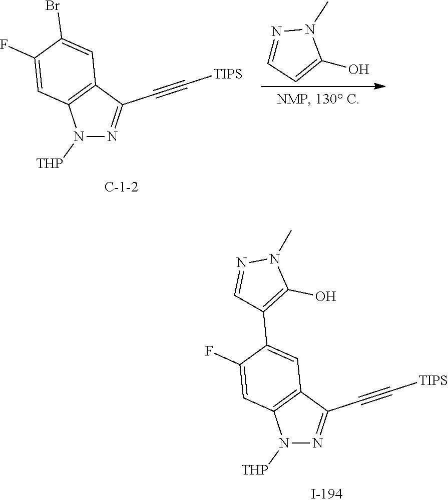

Step 1. To a solution of 2-(5-bromo-6-fluoro-1-tetrahydropyran-2-yl-indazol-3-yl)ethynyl-triisopropyl-silane (1.20 g, 2.50 mmol, 1 eq) in NMP (20 mL) was added 2-methylpyrazol-3-ol (295 mg, 3.00 mmol, 1.2 eq) and K₂CO₃ (692 mg, 5.01 mmol, 2 eq), then the mixture was added [2-(2-aminophenyl)phenyl]-methylsulfonyloxy-palladium;ditert-butyl-[3,6-dimethoxy-2-(2,4,6-triisopropylphenyl) phenyl]phosphane (107 mg, 0.125 mmol, 0.05 eq), degassed and purged with N₂ for 3 times. On completion, the mixture was stirred at 130° C. for 0.5 h under N₂ atmosphere to give 4-[6-fluoro-1-tetrahydropyran-2-yl-3-(2-triisopropylsilylethynyl) indazol-5-yl]-2-methyl-pyrazol-3-ol (1.2 g, crude) as a brown liquid in NMP (20 mL) used to next step. LCMS: m/z 497.3(M+1)

Ex. 194 and 195 were isolated from prep-HPLC following preparations described in General Method S using I-194 in step 3.

### Preparation of [(1R)-2-[[2-[2-[tert-butyl(dimethyl)silyl]oxyethyl]-5-ethoxy-4-iodo-pyrazol-3-yl]methyl-cyclopropyl-amino]-1-methyl-ethyl]methanesulfonate, I-196

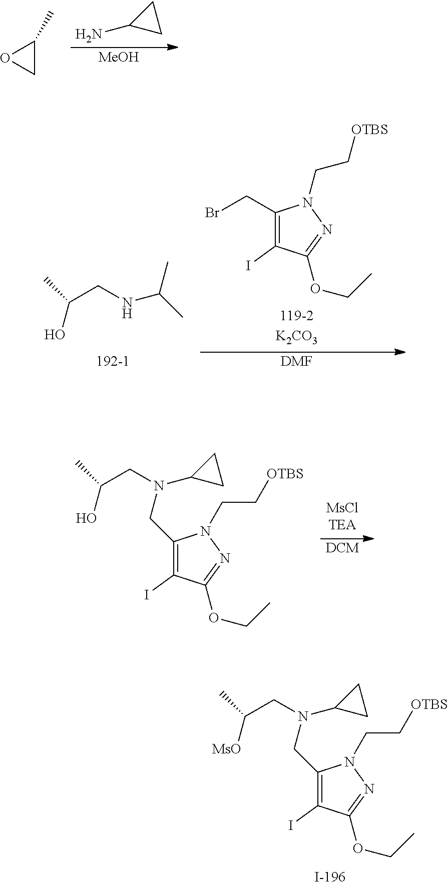

I-196 was synthesized according to General Method S using cyclopropylamine, 192-1, and 119-2.

Ex. 196, 197, and 198 were isolated from synthesis described in General Method S from I-196 and 188-1 in step 4.

Ex. 199 was prepared from 191-1 and 147-1 following preparations similar to those described in the synthesis of Ex. 191.

### Preparation of (2S)-2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol (Ex. 200)

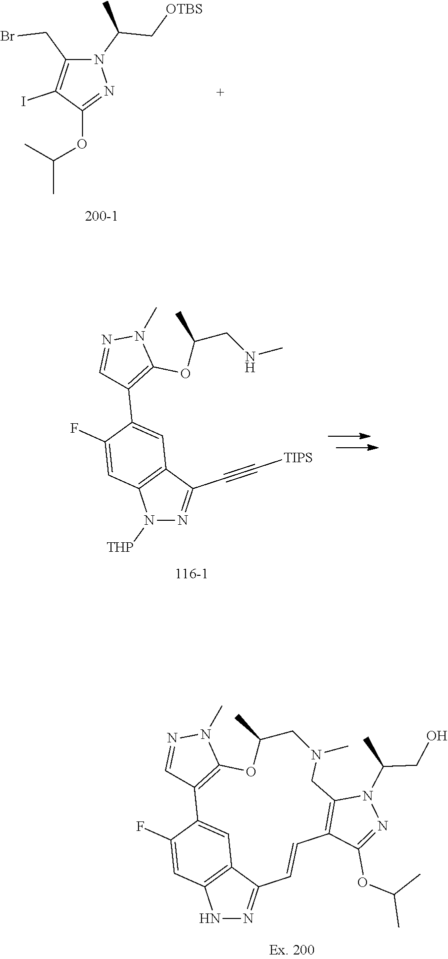

200-1 was prepared in a manner similar to those described in the synthesis of I-155.

Ex. 200 was prepared from 200-1 and 116-1 following the procedures described in General Method M.

### Preparation of 2-{(11S,17E)-6,8,11-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 201) and 2-{(10R,17E)-6,8,10-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 202)

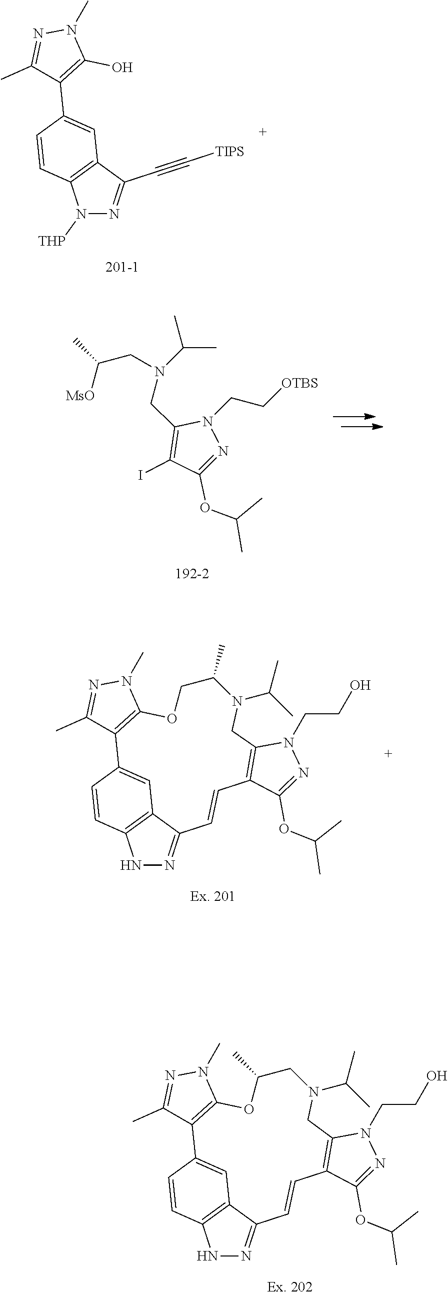

201-1 was prepared from C-1-2 and 2,5-dimethylpyrazol-3-ol following procedure described in the synthesis of 188-1.

201-1 and 192-2 were combined in a manner similar to those described in General Method S to afford inverted product Ex. 201 and unexpected double inverted, Ex. 202.

Ex. 203 was prepared via methods described in General Method R from starting material, Ex. 186.

201-1 was reacted with I-196 in a manner described in General Method S to afford inverted product Ex. 204 and unexpected double inverted, Ex. 205.

### Preparation of (10S,17E)-16-ethoxy-12-ethyl-19-fluoro-8,10,14-trimethyl-2,10,11,12,13,14-hexahydro-8H-3,5-ethenotripyrazolo[3,4-f:3′,4′-j:4″,3″-n][1,4]oxazacyclopentadecine (Ex. 206)

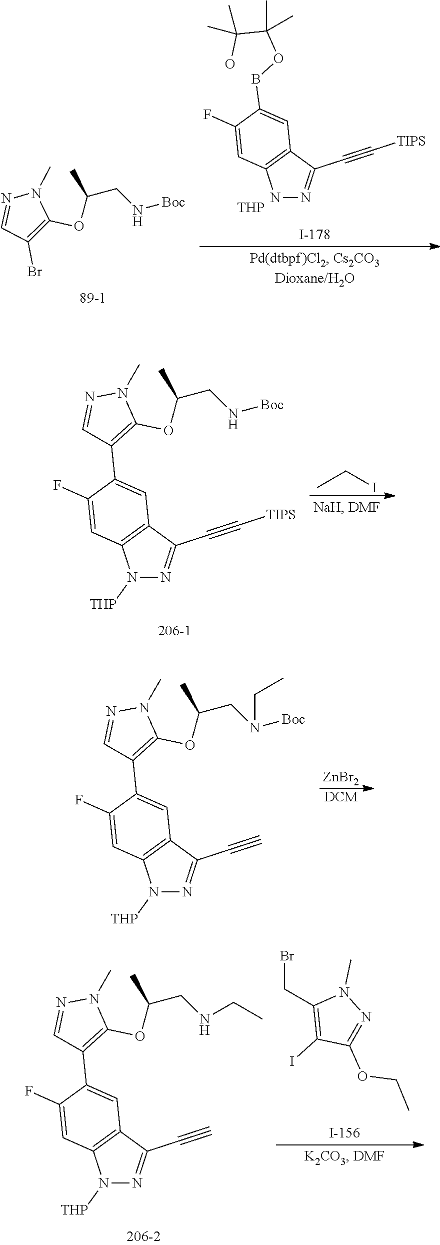

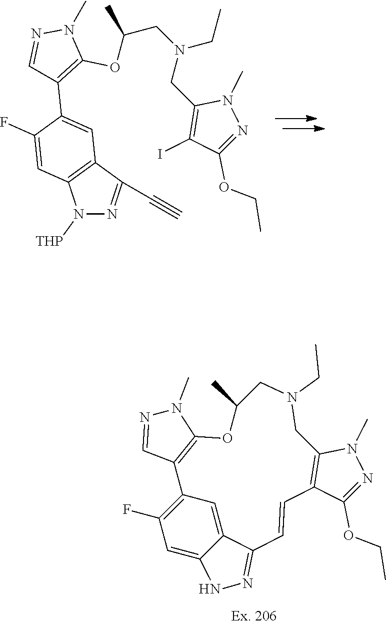

Step 1. To a solution of tert-butyl N-[(2S)-2-(4-bromo-2-methyl-pyrazol-3-yl)oxypropylκarbamate (1.50 g, 4.49 mmol, 1 eq) and 2-δ-fluoro-1-tetrahydropyran-2-yl-5-(4,4,5,5-tetramethyl-1,3,2-dioxaborolan-2-yl) indazol-3-yl]ethynyl-triisopropyl-silane (2.60 g, 4.94 mmol, 1.1 eq) in dioxane (30 mL) and H₂O (6 mL) was added dicesium;carbonate (4.39 g, 13.4 mmol, 3 eq) and ditert-butyl(cyclopentyl)phosphane;dichloropalladium;iron (292 mg, 0.449 mmol, 0.1 eq). The mixture was stirred at 80° C. for 1 h under N₂. On completion, the mixture was quenched with water (20 mL) and extracted with ethyl acetate (25 mL×3), the combined organic phase was dried over anhydrous sodium sulfate, filtered and concentrated to give a residue. The residue was purified by column chromatography (SiO₂, Petroleum ether/Ethyl acetate=1:0 to 3:1) to give tert-butyl N-[(2S)-2-[4-[6-fluoro-1-tetrahydropyran-2-yl-3-(2-triisopropylsilylethynyl) indazol-5-yl]-2-methyl-pyrazol-3-yl]oxypropyl]carbamate (2.54 g, 3.88 mmol, 87% yield) as a brown oil. LCMS: m/z 654.8 (M+1)

Step 2. To a solution of tert-butyl N-[(2S)-2-[4-[6-fluoro-1-tetrahydropyran-2-yl-3-(2-triisopropylsilylethynyl) indazol-5-yl]-2-methyl-pyrazol-3-yl]oxypropyl]carbamate (2.20 g, 3.36 mmol, 1 eq) in DMF (22 mL) was added NaH (538 mg, 13.5 mmol, 60% purity, 4 eq) stirred at 0° C. for 30 min. Then the mixture was added CH₃CH₂I (1.05 g, 6.73 mmol, 2 eq) at 0° C. and stirred at 25° C. for 1 h. On completion, the mixture was quenched with sat. NH₄Cl (40 ml) and extracted with ethyl acetate (25 mL×3), the combined organic phase was dried over anhydrous sodium sulfate, filtered and concentrated to give a residue. The residue was purified by column chromatography (SiO₂, PE:THF=1:0 to 3:1) to give tert-butyl N-ethyl-N-[(2S)-2-[4-(3-ethynyl-6-fluoro-1-tetrahydropyran-2-yl-indazol-5-yl)-2-methyl-pyrazol-3-yl]oxypropyl]carbamate (739 mg, 1.41 mmol, 42% yield) as a yellow solid. LCMS: m/z 548.2 (M+1)

The remaining steps were conducted similarly those described in General Method Q steps 5 and 7-9 to afford Ex. 206.

Ex. 207 was prepared similarly to General Method S from 192-1, 200-1, and then 188-1 at the appropriate steps and assigned as the double inverted by product.

Ex. 208 was prepared from Ex. 184 using procedure described in Ex. 159 step 5 and final deprotection described in General Method N.

Ex. 209 was prepared from 133-1 and 148-1 similarly to General Method Q.

Ex. 210 was prepared following General Method Q from 186-2 and 116-1.

Ex. 210 was converted to Ex. 211 in a similar manner described in General Method R.

Ex. 212 and Ex. 213 were prepared similarly to that of General Method S from 188-1 and the appropriate mesylate synthesized from 147-1 and cyclopropylamine.

### Preparation of [2-[(1S)-2-[tert-butyl (dimethyl) silyl]oxy-1-methyl-ethyl]-5-ethoxy-4-iodo-pyrazol-3-yl]methyl methanesulfonate, I-214

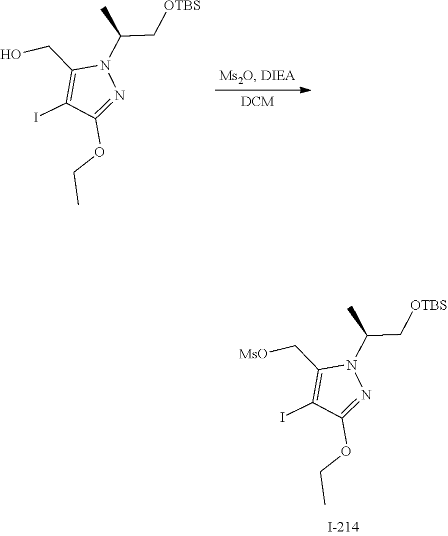 [SMILES: CCOc1nn([C@@H](C)CO[Si](C)(C)C(C)(C)C)c(CO)c1I ; CCOc1nn([C@@H](C)CO[Si](C)(C)C(C)(C)C)c(COS(C)(=O)=O)c1I]

To a solution of 12-1(1S)-2-|tert-butyl(dimethyl)silyl|oxy-1-methyl-ethyl|-5-ethoxy-4-iodo-pyrazol-3-yl]methanol (3.00 g, 6.81 mmol, 1.00 eq, from I-155) in DCM (30 mL) was added methylsulfonyl methanesulfonate (2.37 g, 13.6 mmol, 2.00 eq) and DIEA (2.64 g, 20.4 mmol, 3.00 eq) at 0° C. The mixture was stirred at 25° C. for 2 h. On completion, the reaction was concentrated under vacuum to give [2-[(1S)-2-[tert-butyl (dimethyl) silyl]oxy-1-methyl-ethyl]-5-ethoxy-4-iodo-pyrazol-3-yl]methyl methanesulfonate (9.00 g, crude) as a yellow solid.

Ex. 214 was obtained from 1-214 and 116-1 following procedures detailed in the General Method Q.

Ex. 215 was prepared following procedures described in the synthesis of Ex. 133 steps 1-5 and then General Method L from the following starting materials and intermediates: I-2-1, 1-178, and I-155.

Ex. 216 was prepared following procedures described in the synthesis of Ex. 133 steps 1-5 and then General Method L from the following starting materials and intermediates: I-2-1, 1-178, and 147-1.

### Preparation of 1-[(10R,17E)-16-ethoxy-14-(2-hydroxyethyl)-8,10-dimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one (Ex. 217)

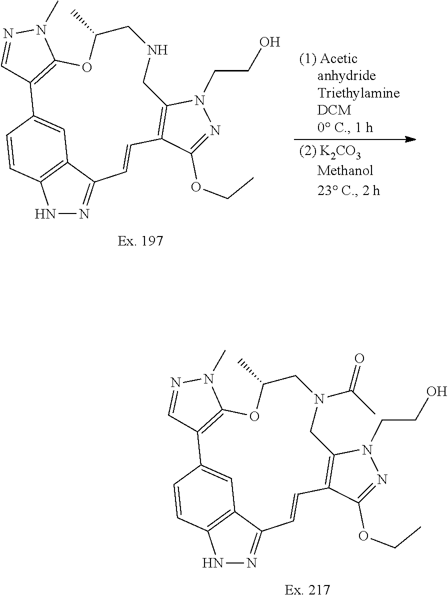 [SMILES: CCOc1nn(CCO)c2c1/C=C/c1n[nH]c3ccc(cc13)-c1cnn(C)c1O[C@H](C)CNC2 ; CCOc1nn(CCO)c2c1/C=C/c1n[nH]c3ccc(cc13)-c1cnn(C)c1O[C@H](C)CN(C(C)=O)C2]

Step 1 was conducted according to General Method J and gave a mixture of over acetylated products. The mixture was taken forward to step 2.

Step 2. To the mixture in Methanol (1 mL) was added Potassium carbonate (35.43 mg, 0.256 mmol) and the suspension was stirred for 2 h at 23° C. The mixture was then diluted with water (0.3 mL) and extracted with DCM (3×1 mL). The combined organic layers were washed with brine, dried over anhydrous sodium sulfate and concentrated under vacuum. The residue was purified by preparative HPLC (C18, 21 mm ×100 mm, MeCN in water with 0.035% TFA) to furnish Ex. 217 (1.04 mg, 0.002 mmol, 39% yield, TFA).

### Preparation of ethyl [(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]acetate (Ex. 218)

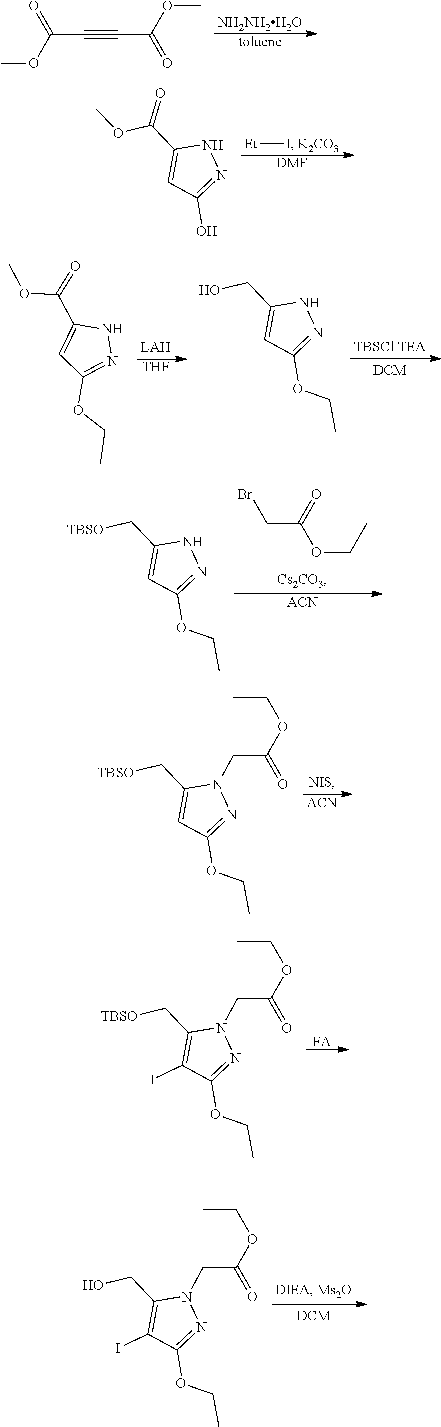

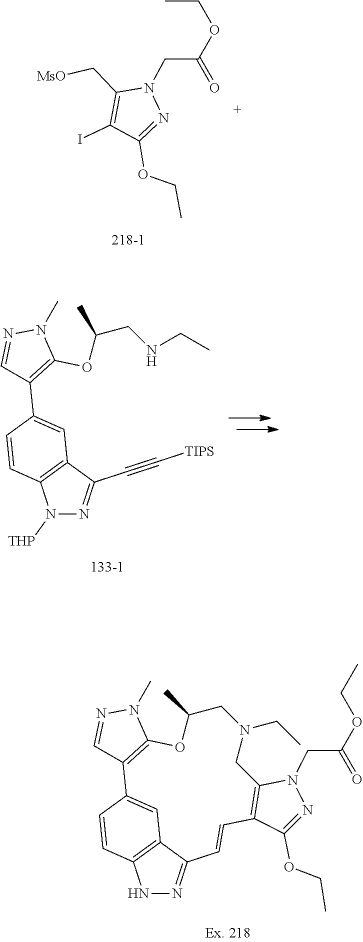

Step 1. The solution of dimethyl but-2-ynedioate (50.0 g, 352 mmol, 1.00 eq) in toluene (250 mL) was added NH₂NH₂—H₂O (23.4 g, 457 mmol, 22.6 mL, 98% purity, 1.30 eq). The mixture was stirred at 25° C. for 2 hr. On completion, the mixture was filtrated and concentrated under reduced pressure to remove solvent and get crude product. The crude product was washed with PE (250 mL) and triturated with Methanol (200 mL) at 20° C. for 20 min to give methyl 3-hydroxy-1H-pyrazole-5-carboxylate (40.5 g, 285 mmol, 81% yield) as light-yellow solid. ¹H NMR (400 MHz, DMSO-d₆) δ=5.80 (s, 1H), 3.78-3.48 (m, 3H). LCMS: m/z 143.2 (M+1)

Step 2. To a solution of methyl 3-hydroxy-1H-pyrazole-5-carboxylate (20.0 g, 141 mmol, 1.00 eq) in DMF (200 mL) was added K₂CO₃ (58.4 g, 422 mmol, 3.00 eq) and iodoethane (22.0 g, 140 mmol, 11.3 mL, 1.00 eq). The mixture was stirred at 80° C. for 4 hr. On completion, the reaction mixture was partitioned between ethyl acetate (50 mL×5) and water (300 mL), the combined organic phase was washed with water (100×2) and dried over anhydrous sodium sulfate, filtered and concentrated to give a residue. The residue was purified by column chromatography (SiO₂, Petroleum ether/Tetrahydrofuran=1/1 to 0/1) to give methyl 3-ethoxy-1H-pyrazole-5-carboxylate (17.7 g, 104 mmol, 74% yield) as colorless oil. ¹H NMR (400 MHz, DMSO-d₆) δ=13.22-12.96 (m, 1H), 6.34-6.08 (m, 1H), 4.19-4.07 (m, 2H), 3.81 (s, 3H), 1.29 (t, J=7.2 Hz, 3H). LCMS: m/z 171.1 (M+1)

Step 3. To a solution of methyl 3-ethoxy-1H-pyrazole-5-carboxylate (17.0 g, 99.9 mmol, 1.00 eq) in THE (250 mL) was added LAH (7.58 g, 200 mmol, 2.00 eq) at 0° C. The mixture was stirred at 0° C. for 6 hr. On completion, the reaction mixture was diluted with EA (50 mL), quenched with H₂O (8 mL) and adjusted with 15% NaOH (8 mL). The combined organic layers were dried over Na₂SO₄ with stirring 10 min at 25° C., filtered and concentrated under reduced pressure to give (3-ethoxy-1H-pyrazol-5-yl)methanol (4.30 g, 30.3 mmol, 30% yield) as colorless oil. ¹H NMR (400 MHz, DMSO-d₆) δ=11.71 (br s, 1H), 5.52 (s, 1H), 5.29-5.12 (m, 1H), 4.41-4.35 (m, 2H), 4.06-4.03 (m, 2H), 1.26 (s, 3H).

Step 4. To a solution of (3-ethoxy-1H-pyrazol-5-yl) methanol (4.30 g, 30.3 mmol, 1.00 eq) in DCM (50 mL) was added TBSCl (5.47 g, 36.3 mmol, 4.45 mL, 1.20 eq) and TEA (9.18 g, 90.8 mmol, 12.6 mL, 3.00 eq). The mixture was stirred at 25° C. for 8 hr. On completion, the reaction mixture was concentrated under reduced pressure to give a residue. The residue was purified by column chromatography (SiO₂, Petroleum ether/Tetrahydrofuran=3/1 to 1/1) to give tert-butyl-[(3-ethoxy-1H-pyrazol-5-yl) methoxy]-dimethyl-silane (4.60 g, 17.9 mmol, 59% yield) as colorless oil. ¹H NMR (400 MHz, DMSO-d₆) δ=11.74 (br s, 1H), 5.54 (d, J=2.0 Hz, 1H), 4.59-4.53 (m, 2H), 4.09-4.02 (m, 2H), 1.26 (t, J=7.2 Hz, 3H), 0.89-0.85 (m, 9H), 0.07-0.03 (m, 6H). LCMS: m/z 257.1 (M+1)

Step 5. To a solution of tert-butyl-[(3-ethoxy-1H-pyrazol-5-yl)methoxy]-dimethyl-silane (5.47 g, 21.3 mmol, 1.00 eq) in ACN (50 mL) was added ethyl 2-bromoacetate (5.35 g, 32.0 mmol, 3.54 mL, 1.50 eq) and Cs₂CO₃ (20.9 g, 64.0 mmol, 3.00 eq). The mixture was stirred at 80° C. for 2.5 hr. On completion, the mixture was filtered, washed with ethyl acetate (50 mL) and concentrated in vacuum to give a residue. The residue was purified by column chromatography (SiO₂, Petroleum ether/Ethyl acetate=4/1 to 1/1) to give ethyl 2-[5-[[tert-butyl (dimethyl) silyl]oxymethyl]-3-ethoxy-pyrazol-1-yl]acetate (5.82 g, 17.0 mmol, 80% yield) as colorless oil. ¹H NMR (400 MHz, DMSO-d₆) δ=5.64 (s, 1H), 4.80 (s, 2H), 4.58 (s, 2H), 4.15-4.01 (m, 4H), 1.29-1.16 (m, 6H), 0.88-0.84 (m, 9H), 0.05 (s, 6H). LCMS: m/z 343.1 (M+1)

Step 6. To a solution of ethyl 2-[5-[[tert-butyl(dimethyl)silyl]oxymethyl]-3-ethoxy-pyrazol-1-yl]acetate (5.76 g, 16.8 mmol, 1.00 eq) in ACN (90 mL) was added NIS (3.78 g, 16.8 mmol, 1.00 eq) at 0° C. The mixture was stirred at 0° C. for 4 hr. On completion, the mixture was quenched with saturated solution of Na₂SO₃ (12 mL) and partitioned between ethyl acetate (30 mL×3) and water (90 mL), the combined organic phase was dried over anhydrous sodium sulfate, filtered and concentrated to give a residue. The residue was purified by column chromatography (SiO₂, Petroleum ether/Tetrahydrofuran=4/1 to 3/1) to give ethyl 2-[5-[[tert-butyl (dimethyl) silyl]oxymethyl]-3-ethoxy-4-iodo-pyrazol-1-yl]acetate (7.10 g, 15.2 mmol, 90% yield) as colorless oil. ¹H NMR (400 MHz, DMSO-d₆) δ=4.94-4.89 (m, 2H), 4.60 (s, 2H), 4.13 (qd, J=7.2, 9.2 Hz, 4H), 1.32-1.27 (m, 3H), 1.22-1.17 (m, 3H), 0.87-0.84 (m, 9H), 0.08-0.04 (m, 6H). LCMS: m/z 468.9 (M+1)

Step 6. A solution of ethyl 2-|5-∥tert-butyl (dimethyl) silyl|oxymethyl|-3-ethoxy-4-iodo-pyrazol-1-yl]acetate (6.84 g, 14.6 mmol, 1.00 eq) in HCOOH (35 mL) was stirred at 25° C. for 7 hr. On completion, the reaction mixture was partitioned between ethyl acetate (200 mL) and water (200 mL), the combined organic phase was washed with water (60 mL×3), dried over anhydrous sodium sulfate, filtered and concentrated to give a residue. The residue was purified by column chromatography (SiO₂, Petroleum ether/Tetrahydrofuran=5/1 to 1/1) to give ethyl 2-[3-ethoxy-5-(hydroxymethyl)-4-iodo-pyrazol-1-yl]acetate (1.82 g, 5.14 mmol, 35% yield) as white solid. ¹H NMR (400 MHz, DMSO-d6) δ=5.39 (t, J=5.6 Hz, 1), 4.94 (s, 2H), 4.38 (d, J=5.6 Hz, 2H), 4.13 (dq, J=1.6, 7.2 Hz, 4H), 1.28 (t, J=7.2 Hz, 3H), 1.19 (t, J=7.2 Hz, 3H). LCMS: m/z 355.0 (M+1)

Step 7. To a solution of ethyl 2-[3-ethoxy-5-(hydroxymethyl)-4-iodo-pyrazol-1-yl]acetate (500 mg, 1.41 mmol, 1.00 eq) in DCM (5 mL) was added DIEA (547 mg, 4.24 mmol, 738 μL, 3.00 eq) firstly and methylsulfonyl methanesulfonate (492 mg, 2.82 mmol, 2.00 eq) at 0° C. The mixture was stirred at 25° C. for 2 hr. On completion, the reaction mixture was concentrated under reduced pressure to give ethyl 2-[3-ethoxy-4-iodo-5-(methylsulfonyloxymethyl) pyrazol-1-yl]acetate (610 mg, crude) as brown oil. LCMS: m/z 432.8 (M+1) [01772]2-[3-ethoxy-4-iodo-5-(methylsulfonyloxymethyl) pyrazol-1-yl]acetate was converted to Ex. 218 after combining with 133-1 following procedures described in the synthesis of Ex. 191.

### Preparation of (S)-(1-(1-((tert-butyldimethylsilyl)oxy)propan-2-yl)-4-iodo-3-isopropoxy-1H-pyrazol-5-yl)methyl methanesulfonate, I-219

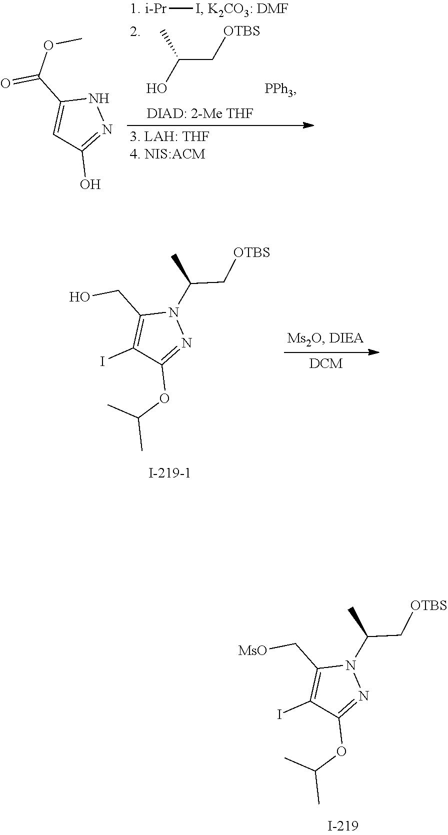

Steps 1 to 4 were conducted in a manner similar to those described in the synthesis of 1-155.

Step 5 was conducted in a manner similar to that described in the synthesis of I-214. [01776]1-219 was combined with 206-2 following procedures described in General Method M and Q to afford Ex. 219.

### Preparation of (2S)—N-[[2-[2-[tert-butyl(dimethyl)silyl]oxyethyl]-4-iodo-5-isopropoxy-pyrazol-3-yl]methyl]-2-chloro-N-methyl-propanamide, 1-220

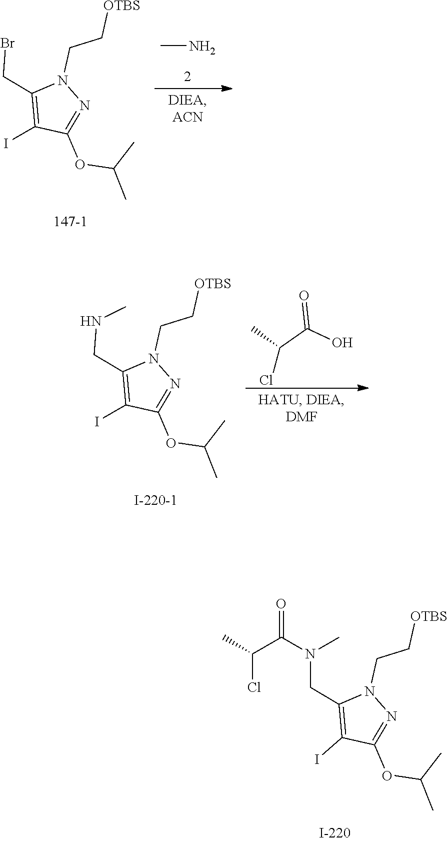

Step 1. To a solution of 2-[5-(bromomethyl)-4-iodo-3-isopropoxy-pyrazol-1-yl]ethoxy-tert-butyl-dimethyl-silane (2.00 g, 3.97 mmol, 1 eq) and methanamine (536 mg, 7.95 mmol, 2 eq, HCl) in ACN (20 mL) was added DIEA (7.95 mmol, 1.4 mL, 2 eq). The mixture was stirred at 60° C. for 0.5 hours. On completion, the mixture was quenched with water (20 mL) and extracted with ethyl acetate (25 mL×3), the combined organic phase was dried over anhydrous sodium sulfate, concentrated to give a residue. The residue was purified by column chromatography (SiO₂, Petroleum ether/Ethyl acetate=1:0 to 0:1) to give 1-[2-[2-[tert-butyl(dimethyl)silyl]oxyethyl]-4-iodo-5-isopropoxy-pyrazol-3-yl]-N-methyl-methanamine (660 mg, 1.46 mmol, 36% yield) as colorless oil. LCMS: m/z 454.4 (M+1)

Step 2. To a solution of 1-[2-[2-[tert-butyl(dimethyl)silyl]oxyethyl]-4-iodo-5-isopropoxy-pyrazol-3-yl]-N-methyl-methanamine (610 mg, 1.35 mmol, 1 eq) and (2S)-2-chloropropanoic acid (218 mg, 2.02 mmol, 1.5 eq) in DMF (5 mL) was added HATU (1.02 g, 2.69 mmol, 2 eq) and DIEA (521 mg, 4.04 mmol, 3 eq). The mixture was stirred at 25° C. for 0.5 hours. On completion, the mixture was concentrated to give a residue. The residue was purified by column chromatography (SiO₂, Petroleum ether/Ethyl acetate=1:0 to 1:1) to give (2S)—N-[[2-[2-[tert-butyl(dimethyl)silyl]oxyethyl]-4-iodo-5-isopropoxy-pyrazol-3-yl]methyl]-2-chloro-N-methyl-propanamide, I-220 (200 mg, 0.367 mmol, 27% yield) as yellow oil. ¹H NMR (400 MHz, CDCl₃) δ=4.90-4.79 (m, 1H), 4.78-4.66 (m, 2H), 4.64-4.50 (m, 1H), 4.20-4.08 (m, 2H), 3.93-3.76 (m, 2H), 3.12-2.80 (m, 3H), 1.76-1.63 (m, 3H), 1.38-1.35 (m, 6H), 0.83-0.79 (m, 9H), −0.04-0.10 (m, 6H). LCMS: m/z 544.2 (M+1)

Ex. 220 was prepared from I-220 and 188-1 in a manner similar to those described in General Method M.

### Preparation of 2-[(10S,17E)-16-{[1-(methanesulfonyl)azetidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 221)

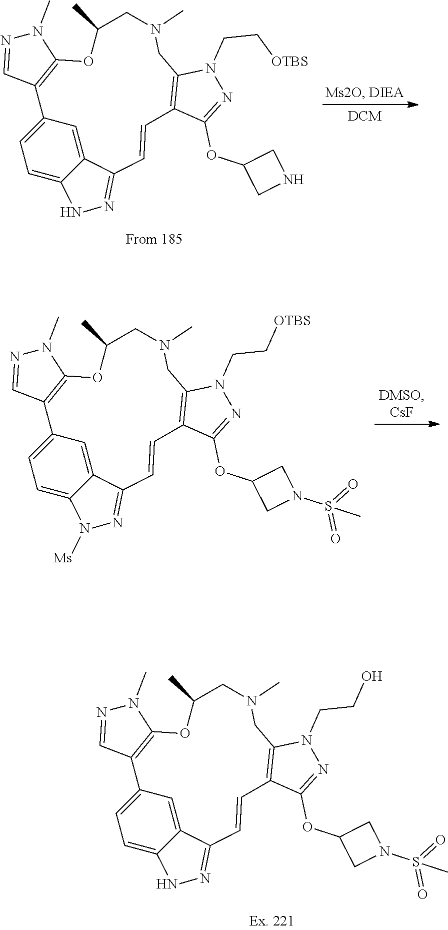 [SMILES: C[C@H]1CN(C)Cc2c(c(OC3CNC3)nn2CCO[Si](C)(C)C(C)(C)C)/C=C/c2n[nH]c3ccc(cc23)-c2cnn(C)c2O1 ; C[C@H]1CN(C)Cc2c(c(OC3CN(S(C)(=O)=O)C3)nn2CCO[Si](C)(C)C(C)(C)C)/C=C/c2nn(S(C)(=O)=O)c3ccc(cc23)-c2cnn(C)c2O1 ; C[C@H]1CN(C)Cc2c(c(OC3CN(S(C)(=O)=O)C3)nn2CCO)/C=C/c2n[nH]c3ccc(cc23)-c2cnn(C)c2O1]

Step 1 was conducted according to the synthesis of I-214 starting with intermediate from the synthesis of Ex. 185.

Step 2. To a solution of tert-butyl-dimethyl-[2-[(8S,17E)-5,8,10-trimethyl-21-methylsulfonyl-15-(1-methylsulfonylazetidin-3-yl)oxy-7-oxa-4,5,10,13,14,20,21-heptazapentacyclo [17.5.2.02,6.012,16.022,26]hexacosa-1(24),2(6),3,12(16),14,17,19,22,25-nonaen-13-yl]ethoxy]silane (50.0 mg, 0.065 mmol, 1.00 eq) in DMSO (0.5 mL) was added CsF (29.4 mg, 0.193 mmol, 3.00 eq). The mixture was stirred at 25° C. for 1 h. On completion, the mixture was filtered and concentrated under reduced pressure to give a residue. The residue was purified by Prep-HPLC (column: Phenomenex luna C18 150*25 mm*10 um; mobile phase: [water(FA)-ACN];gradient:6%-36% B over 10 min) to give 2-[(8S,17E)-5,8,10-trimethyl-15-(1-methylsulfonylazetidin-3-yl)oxy-7-oxa-4,5,10,13,14,20,21-heptazapentacyclo[17.5.2.02,6.012,16.022,26]hexacosa-1(24),2(6),3,12(16),14,17,19,22,25-nonaen-13-yl]ethanol, Ex. 221 (1.89 mg, 0.003 mmol, 5% yield) as an off-white solid.

Ex. 222 was prepared from Ex. 218 following procedures described in Ex. 105.

### Preparation of 1-[2-[2-[tert-butyl(dimethyl)silyl]oxyethyl]-4-iodo-5-isopropoxy-pyrazol-3-yl]ethenone, I-223

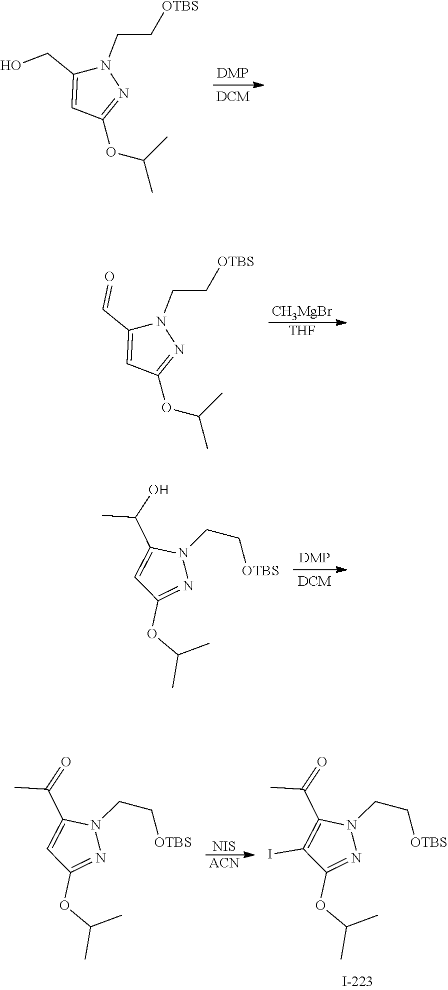

Step 1. To a solution of [2-[2-[tert-butyl(dimethyl)silyl]oxyethyl]-5-isopropoxy-pyrazol-3-yl]methanol (1 g, 3.18 mmol, 1 eq. from Ex. 147) in DCM (15 mL) was added DMP (1.62 g, 3.82 mmol, 1.2 eq). The mixture was stirred at 25° C. for 4 hours. On completion, the mixture was filtered and concentrated to give 2-[2-[tert-butyl(dimethyl)silyl]oxyethyl]-5-isopropoxy-pyrazole-3-carbaldehyde (800 mg, 2.56 mmol, 80% yield) as yellow oil. ¹H NMR (400 MHz, CDCl₃) δ=9.78 (s, 1H), 6.22-6.21 (m, 1H), 4.73 (td, J=6.0, 12.3 Hz, 1H), 4.52-4.47 (m, 2H), 3.90 (t, J=5.6 Hz, 2H), 1.36 (d, J=6.0 Hz, 6H), 0.84-0.81 (m, 9H), −0.03-0.08 (m, 6H). LCMS: m/z 313.4 (M+1)

Step 2. A solution of 2-[2-[tert-butyl(dimethyl)silyl]oxyethyl]-5-isopropoxy-pyrazole-3-carbaldehyde (800 mg, 2.56 mmol, 1 eq) in THE (10 mL) at 0° C. The mixture was degassed and purged with N₂ for 3 times, and then bromo(methyl)magnesium (3 M, 1 mL, 1.5 eq) was added dropwise at 0° C. The mixture was stirred at 25° C. for 1 hour under N₂ atmosphere. On completion, the reaction mixture was partitioned between ethyl acetate (10 mL×3) and water (10 mL), the combined organic phase was dried over anhydrous sodium sulfate, filtered and concentrated to give 1-[2-[2-[tert-butyl(dimethyl)silyl]oxyethyl]-5-isopropoxy-pyrazol-3-yl]ethanol (700 mg, 2.13 mmol, 83% yield) as yellow oil. ¹H NMR (400 MHz, CDCl₃) δ=5.58 (s, 1H), 4.83 (q, J=6.4 Hz, 1H), 4.72-4.62 (m, 1H), 4.21-4.15 (m, 2H), 4.05-3.98 (m, 1H), 3.93-3.88 (m, 1H), 3.78-3.73 (m, 1H), 1.53 (d, J=6.4 Hz, 3H), 1.34 (d, J=6.4 Hz, 6H), 0.83 (d, J=0.8 Hz, 9H), 0.01 (d, J=1.6 Hz, 6H). LCMS: m/z 329.2 (M+1)

Step 3. To a solution of 1-[2-[2-[tert-butyl(dimethyl)silyl]oxyethyl]-5-isopropoxy-pyrazol-3-yl]ethanol (640 mg, 1.95 mmol, 1 eq) in DCM (1 mL) was added DMP (991 mg, 2.34 mmol, 1.2 eq). The mixture was stirred at 25° C. for 4 hours. On completion, the reaction mixture was partitioned between DCM (10 mL×3) and water (10 mL), the combined organic phase was dried over anhydrous sodium sulfate, filtered and concentrated to give 1-|2-|2-|tert-butyl(dimethyl)silylloxyethyl|-5-isopropoxy-pyrazol-3-yl]ethanone (550 mg, 1.68 mmol, 86% yield) as yellow solid. ¹H NMR (400 MHz, CDCl₃) δ=6.14 (s, 1H), 4.79-4.71 (m, 1H), 4.56-4.51 (m, 2H), 3.90-3.85 (m, 2H), 2.45 (s, 1H), 1.35 (d, J=6.0 Hz, 6H), 0.84-0.82 (m, 9H), −0.02-0.05 (m, 6H). LCMS: m/z 327.2 (M+1)

Step 4. To a solution of 1-[2-[2-[tert-butyl(dimethyl)silyl]oxyethyl]-5-isopropoxy-pyrazol-3-yl]ethanone (500 mg, 1.53 mmol, 1 eq) in ACN (6 mL) was added NIS (413 mg, 1.84 mmol, 1.2 eq). The mixture was stirred at 0° C. for 14 hours. On completion, the reaction mixture was partitioned between ethyl acetate (10 mL×3) and water (10 mL), the combined organic phase was dried over anhydrous sodium sulfate, filtered and concentrated to give 1-[2-[2-[tert-butyl(dimethyl)silyl]oxyethyl]-4-iodo-5-isopropoxy-pyrazol-3-yl]ethanone (600 mg, 1.33 mmol, 86% yield) as yellow oil. ¹H NMR (400 MHz, CDCl₃) δ=4.92-4.84 (m, 1H), 4.48 (t, J=5.6 Hz, 2H), 3.84 (t, J=5.6 Hz, 2H), 2.74 (s, 3H), 1.39-1.36 (m, 6H), 0.85-0.82 (m, 9H), −0.01-0.05 (m, 6H). LCMS: m/z 453.0 (M+1)

### Preparation of 1-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]-2-methylpropan-2-ol (Ex. 223) and 2-{(10S,13S,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 224)

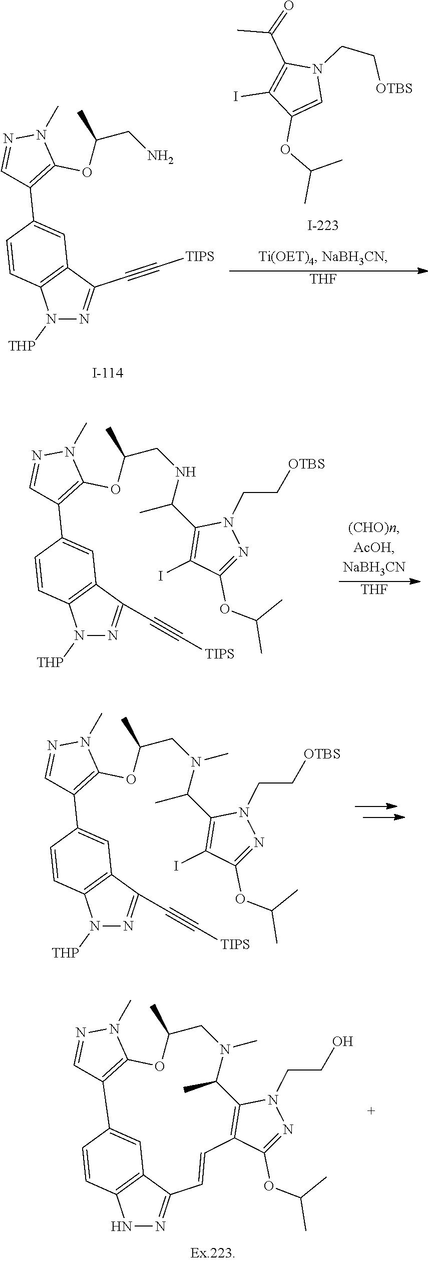

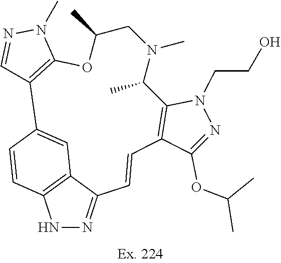 [SMILES: CC(C)Oc1nn(CCO)c2c1/C=C/c1n[nH]c3ccc(cc13)-c1cnn(C)c1O[C@@H](C)CN(C)[C@H]2C]

Step 1. To a mixture of 1-[2-[2-[tert-butyl(dimethyl)silyl]oxyethyl]-4-iodo-5-isopropoxy-pyrazol-3-yl]ethanone (580 mg, 1.28 mmol, 1 eq), (2S)-2-[2-methyl-4-[1-tetrahydropyran-2-yl-3-(2-triisopropylsilylethynyl) indazol-5-yl]pyrazol-3-yl]oxypropan-1-amine (686 mg, 1.28 mmol, 1 eq) in THF (10 mL) was added Ti(OEt)₄ (1.17 g, 5.13 mmol, 4 eq) at 25° C. for 1 hour, then NaBH₃CN (483 mg, 7.69 mmol, 6 eq) was added at 70° C. The mixture was stirred at 70° C. for 13 hours. On completion, the reaction mixture was partitioned between ethyl acetate (20 mL×3) and water (10 mL), the combined organic phase was dried over anhydrous sodium sulfate, filtered and concentrated to give (2S)—N-[1-[2-[2-[tert-butyl(dimethyl)silyl]oxyethyl]-4-iodo-5-isopropoxy-pyrazol-3-yl]ethyl]-2-[2-methyl-4-[1-tetrahydropyran-2-yl-3-(2-triisopropylsilylethynyl) indazol-5-yl]pyrazol-3-yl]oxy-propan-1-amine (600 mg, 0.617 mmol, 48% yield) as yellow solid. ¹H NMR (400 MHz, CDCl₃) δ=7.84-7.74 (m, 1H), 7.66-7.57 (m, 3H), 5.78-5.69 (m, 1H), 4.17-4.09 (m, 5H), 2.05 (s, 7H), 1.56 (s, 24H), 1.39-1.32 (m, 9H), 1.30-1.24 (m, 9H), 0.85-0.81 (m, 9H), 0.02-0.06 (m, 6H). LCMS: m/z 973.3 (M+1)

Step 2. To a solution of (2S)—N-[1-[2-[2-[tert-butyl(dimethyl)silyl]oxyethyl]-4-iodo-5-isopropoxy-pyrazol-3-yl]ethyl]-2-[2-methyl-4-[1-tetrahydropyran-2-yl-3-(2-triisopropylsilylethynyl) indazol-5-yl]pyrazol-3-yl]oxy-propan-1-amine (570 mg, 0.586 mmol, 1 eq) in THE (6 mL) was added AcOH (35.2 mg, 0.586 mmol, 1 eq), (HCHO)n (570 mg) and NaBH₃CN (55.2 mg, 0.879 mmol, 1.5 eq). The mixture was stirred at 25° C. for 14 hours. On completion, the reaction mixture was partitioned between ethyl acetate (10 mL×3) and water (10 mL), the combined organic phase was dried over anhydrous sodium sulfate, filtered and concentrated to give (2S)—N-[1-[2-[2-[tert-butyl(dimethyl)silyl]oxyethyl]-4-iodo-5-isopropoxy-pyrazol-3-yl]ethyl]-N-methyl-2-[2-methyl-4-[1-tetrahydropyran-2-yl-3-(2-triisopropylsilylethynyl) indazol-5-yl]pyrazol-3-yl]oxy-propan-1-amine (420 mg, 0.425 mmol, 72% yield) as yellow oil. ¹H NMR (400 MHz, CDCl₃) δ=7.82-7.66 (m, 2H), 7.61-7.53 (m, 1H), 7.50-7.38 (m, 1H), 5.79-5.69 (m, 1H), 4.88-4.74 (m, 1H), 4.40 (s, 1H), 4.18-4.03 (m, 3H), 3.98-3.85 (m, 4H), 3.80-3.71 (m, 2H), 2.72-2.43 (m, 3H), 2.22-2.09 (m, 2H), 2.03-1.91 (m, 2H), 1.77 (d, J=9.2 Hz, 2H), 1.72-1.64 (m, 1H), 1.36 (d, J=2.8 Hz, 8H), 1.30-1.25 (m, 3H), 1.19 (s, 21H), 1.14-1.01 (m, 3H), 0.82 (d, J=4.0 Hz, 9H), −0.05 (dd, J=2.4, 5.6 Hz, 6H)

(2S)—N-[1-[2-[2-[tert-butyl(dimethyl)silyl]oxyethyl]-4-iodo-5-isopropoxy-pyrazol-3-yl]ethyl]-N-methyl-2-[2-methyl-4-[1-tetrahydropyran-2-yl-3-(2-triisopropylsilylethynyl) indazol-5-yl]pyrazol-3-yl]oxy-propan-1-amine was taken forward in a manner similar to those described in General Method M followed by SFC separation to afford Ex. 223 and Ex. 224.

### Preparation 2-{(17E)-10-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 225) and 2-{(17E)-11-(fluoromethyl)-8,12-dimethyl-16-[(propan-2-yl)oxy]-2,8,10,11.12,13-hexahydro-14H-3,5-ethenotripyrazolo[3,4-f:3′,4′-j:4″,3″-n][1,4]oxazacyclopentadecin-14-yl}ethan-1-ol (Ex. 226)

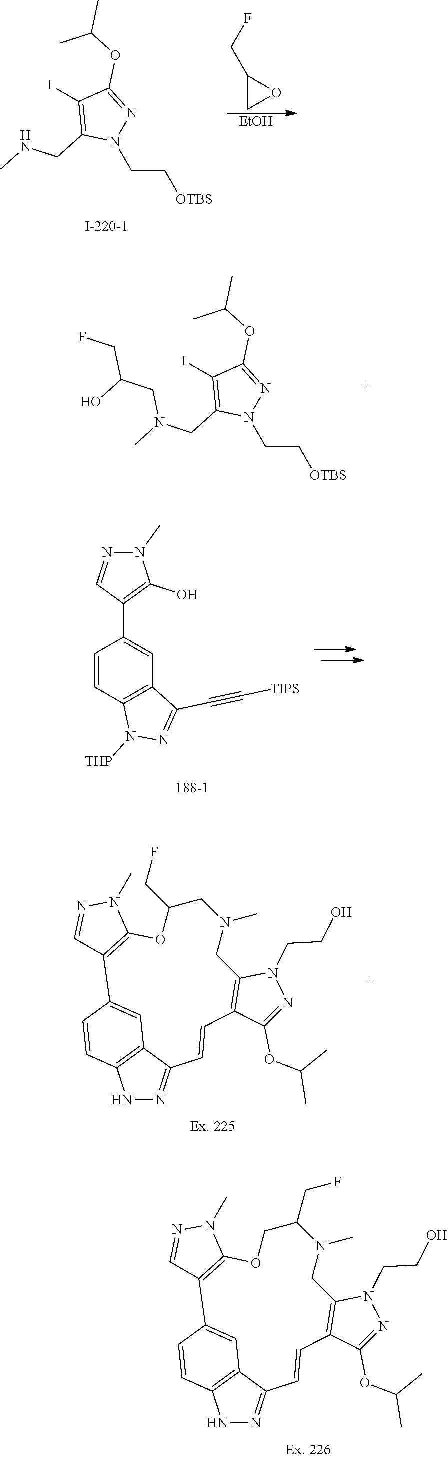

Step 1. To a solution of 2-(fluoromethyl)oxirane (671 mg, 8.82 mmol, 2 eq) and 1-[2-[2-[tert-butyl(dimethyl)silyl]oxyethyl]-4-iodo-5-isopropoxy-pyrazol-3-yl]-N-methyl-methanamine (2.00 g, 4.41 mmol, 1 eq) in EtOH (20 mL) was added. The mixture was stirred at 70° C. for 16 h. On completion, the reaction mixture was filtered and concentrated under reduced pressure to give a residue, the residue was purified by column chromatography (SiO₂, Petroleum ether/Ethyl acetate=100/1 to 5/1) to give 1-[[2-[2-[tert-butyl(dimethyl)silyl]oxyethyl]-4-iodo-5-isopropoxy-pyrazol-3-yl]imethyl-methyl-amino]-3-fluoro-propan-2-ol (1.60 g, 2.93 mmol, 66% yield, 97% purity) as yellow oil. ¹H NMR (400 MHz, CDCl₃) 5=4.88-4.77 (m, 1H), 4.55-4.30 (m, 2H), 4.27-4.11 (m, 2H), 3.94 (d, J=4.0 Hz, 1H), 3.93-3.87 (m, 2H), 3.77-3.55 (m, 2H), 2.68-2.46 (m, 2H), 2.28 (s, 3H), 1.36 (d, J=6.4 Hz, 6H), 0.81 (s, 9H), −0.07 (s, 6H). [01796]1-[[2-[2-[tert-butyl(dimethyl)silyl]oxyethyl]-4-iodo-5-isopropoxy-pyrazol-3-yl]methyl-methyl-amino]-3-fluoro-propan-2-ol was combined with 188-1 according to procedures described in General Method S to afford Ex. 225 and inverted by product Ex. 226.

### Preparation of (S)-5-(bromomethyl)-1-(2-((tert-butyldimethylsilyl)oxy)propyl)-4-iodo-3-isopropoxy-1H-pyrazole, I-227

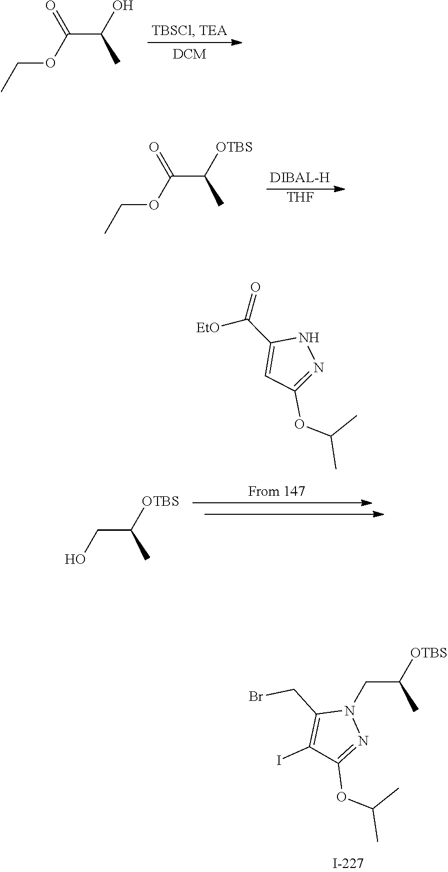

Step 1. To a solution of ethyl (2S)-2-hydroxypropanoate (5.00 g, 42.3 mmol, 1 eq) in DCM (50 mL) was added IMIDAZOLE (8.64 g, 127 mmol, 3 eq), and then TBSCl (9.57 g, 63.5 mmol, 1.5 eq) was added at 0° C. The mixture was stirred at 25° C. for 2 h. On completion, the reaction mixture was partitioned between DCM (60 mL×3) and water (50 mL), the combined organic phase was dried over anhydrous sodium sulfate, filtered and concentrated to give a residue. The residue was purified by column chromatography (SiO₂, PE/EA=10:1 to 10:1) to give ethyl (2S)-2-|tert-butyl(dimethyl)silyl|oxypropanoate (9.70 g, 41.7 mmol, 99% yield) as a brown solid. ¹H NMR (400 MHz, DMSO-d₆) δ=4.38-4.29 (m, 1H), 4.14-4.04 (m, 2H), 1.28 (d, J=6.8 Hz, 3H), 1.19 (t, J=7.2 Hz, 3H), 0.87-0.85 (m, 9H), 0.05 (d, J=3.2 Hz, 6H)

Step 2. To a solution of ethyl (2S)-2-[tert-butyl(dimethyl)silyl]oxypropanoate (8.70 g, 37.4 mmol, 1 eq) in THF (87 mL) was added DIBAL-H (1 M, 74.8 mL, 2 eq) at 0° C. under N₂ atmosphere. The mixture was stirred 25° C. for 4 h. On completion, water (3 mL), 15% NaOH (3 mL) and water (7.5 mL) was added successively, the mixture was filtered and concentrated to give a residue. The residue was purified by column chromatography (SiO₂, Petroleum ether/Ethyl acetate=10:1 to 10:1) to give (2S)-2-[tert-butyl(dimethyl)silyl]oxypropan-1-ol (4.87 g, 25.6 mmol, 68% yield) as a white oil. ¹H NMR (400 MHz, DMSO-d₆) δ=4.53 (t, J=5.6 Hz, 1H), 3.80-3.66 (m, 1H), 3.30-3.09 (m, 2H), 1.09-0.99 (m, 3H), 0.87-0.84 (m, 8H), 0.04 (d, J=0.8 Hz, 6H)

The remaining steps were conducted similarly to those described in Ex. 186-2 using (2S)-2-[tert-butyl(dimethyl)silyl]oxypropan-1-ol and ethyl 3-isopropoxy-1H-pyrazole-5-carboxylate (from Ex. 147) to afford I-227

Ex. 227 was prepared in a manner similar to those described in General Method S from ethylamine, 1-227 in step 2 and 188-1 in step 4.

Inverted Ex. 228 and double inverted Ex. 229 was isolated from Prep-HPLC following syntheses like those described in General Method S from I-214 in step 2 and I-194 in step 4.

Inverted Ex. 230 and double inverted Ex. 231 was isolated from Prep-HPLC following syntheses like those described in General Method S from I-219 in step 2 and I-194 in step 4.

### Preparation of tert-butyl 2-[4-iodo-3-isopropoxy-5-(methylsulfonyloxymethyl) pyrazol-1-yl]acetate, I-232

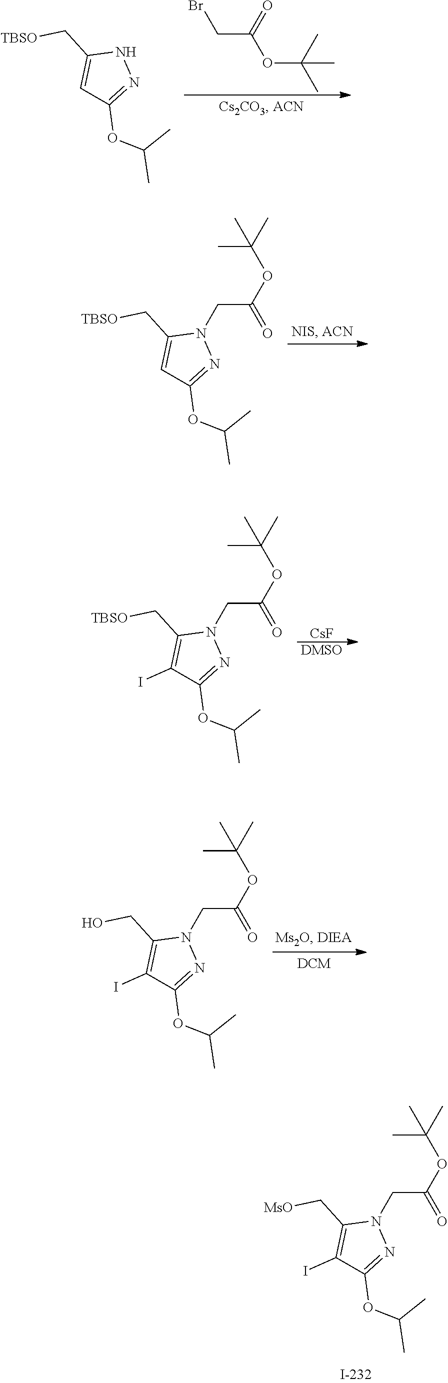

Step 1. To a solution of tert-butyl 2-bromoacetate (25.2 g, 129 mmol, 1.40 eq) in ACN (250 mL) was added tert-butyl-[(3-isopropoxy-1H-pyrazol-5-yl)methoxy]-dimethyl-silane (25.0 g, 92.4 mmol, 1.00 eq, from Ex. 147) and Cs₂CO₃ (90.3 g, 277 mmol, 3.00 eq). The mixture was stirred at 80° C. for 2.5 hr. On completion, the mixture was diluted with water (250 mL) and extracted with ethyl acetate (300 mL*3) and washed with saturated NaCl (300 mL*3), the combined organic phase was dried over anhydrous sodium sulfate, filtered and concentrated to give tert-butyl 2-[5-[[tert-butyl(dimethyl)silyl]oxymethyl]-3-isopropoxy-pyrazol-1-yl]acetate (31.0 g, crude) as a white solid. ¹H NMR (400 MHz, DMSO-d₆) δ=5.74 (s, 1H), 4.67 (s, 2H), 4.64-4.58 (m, 1H), 4.56 (s, 2H), 1.41-1.39 (m, 9H), 1.23 (d, J=6.0 Hz, 6H), 0.86 (s, 9H), 0.05 (s, 6H). LCMS: m/z 385.3 (M+1)

Step 2. To a solution of tert-butyl 2-[5-[[tert-butyl(dimethyl)silyl]oxymethyl]-3-isopropoxy-pyrazol-1-yl]acetate (30.0 g, 78.0 mmol, 1.00 eq) in ACN (300 mL) was added NIS (17.5 g, 78.0 mmol, 1.00 eq) at 0° C. The mixture was stirred at 0° C. for 1 hr. On completion, the mixture was quenched with water (300 mL) and extracted with EA (300 mL *3). The combined organic layers were washed with brine (300 ml *3), dried over Na₂SO₄, filtered and concentrated to give tert-butyl 2-[5-[[tert-butyl(dimethyl)silyl]oxymethyl]-4-iodo-3-isopropoxy-pyrazol-1-yl]acetate (31.0 g, crude) as a red oil. ¹H NMR (400 MHz, DMSO-d₆) (=4.80 (s, 2H), 4.75-4.70 (m, 1H), 4.57 (s, 2H), 1.40 (s, 9H), 1.26 (d, J=6.0 Hz, 6H), 0.87 (s, 9H), 0.08 (s, 6H).

Step 3. To a solution of tert-butyl 2-[5-[[tert-butyl(dimethyl)silyl]oxymethyl]-4-iodo-3-isopropoxy-pyrazol-1-yl]acetate (3.00 g, 5.88 mmol, 1.00 eq) in DMSO (30 mL) was added CsF (2.68 g, 17.6 mmol, 3.00 eq) at 25° C. The mixture was stirred at 25° C. for 1 hr. On completion, the mixture was diluted with water (150 mL) and extracted with ethyl acetate (80 mL*3) and washed with saturated NaCl (100 ml *3), the combined organic phase was dried over anhydrous sodium sulfate, filtered and concentrated to give tert-butyl 2-[5-(hydroxymethyl)-4-iodo-3-isopropoxy-pyrazol-1-yl]acetate (2.28 g, crude) as a yellow oil. ¹H NMR (400 MHz, DMSO-d6) (=5.37 (t, J=5.6 Hz, 1H), 4.81 (s, 2H), 4.75-4.70 (m, 1H), 4.36 (d, J=5.6 Hz, 2H), 1.42-1.41 (m, 9H), 1.26 (d, J=6.0 Hz, 6H).

Step 4. To a solution of tert-butyl 2-[5-(hydroxymethyl)-4-iodo-3-isopropoxy-pyrazol-1-yl]acetate (2.50 g, 6.31 mmol, 1.00 eq) in DCM (25 mL) was added DIEA (3.26 g, 25.2 mmol, 4.40 mL, 4.00 eq) at 0° C. Then methylsulfonylmethanesulfonate (2.20 g, 12.6 mmol, 2.00 eq) was added at 0° C. The mixture was stirred at 25° C. for 1 hr. On completion, the mixture was quenched with water (30 mL) and extracted with DCM (30 mL *3). The combined organic layer was dried over Na₂SO₄, filtered and concentrated to give tert-butyl 2-[4-iodo-3-isopropoxy-5-(methylsulfonyloxymethyl) pyrazol-1-yl]acetate (3.00 g, crude) as a brown oil. LCMS: m/z 496.8 (M+1)

### Preparation of 2-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}acetamide (Ex. 232)

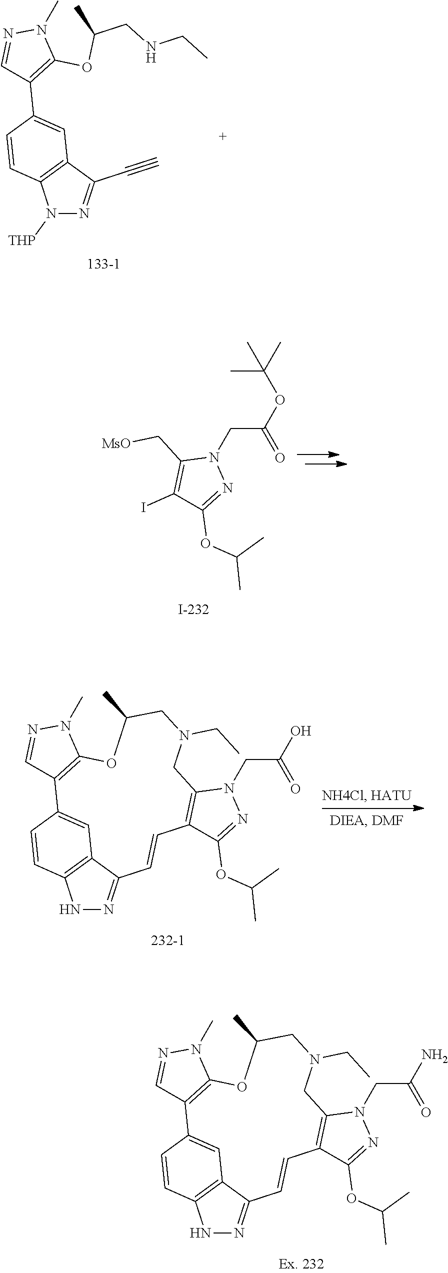

I-232 was combined with 133-1 following procedures similar to those described in General Method M to afford 232-1.

To a solution of 2-[(8S,17E)-10-ethyl-15-isopropoxy-5,8-dimethyl-7-oxa-4,5,10,13,14,20,21-heptazapentacyclo[17.5.2.0^{2,6}.0^{12,16}.0^{22,26}]hexacosa-1(25),2(6),3,12(16),14,17,19,22(26),23-nonaen-13-yl]acetic acid (30.0 mg, 0.058 mmol, 1.00 eq) in THF (1 mL) was added DIEA (14.9 mg, 0.115 mmol, 2.00 eq) and HATU (32.9 mg, 0.087 mmol, 1.50 eq) for 0.5 h, the mixture was added NH₄C1 (3.09 mg, 0.058 mmol, 1.00 eq) stirred at 25° C. for 1 h. On completion, the reaction mixture was concentrated in vacuum. The mixture was purified by Prep-HPLC (column: Phenomenex luna C18 150*25 mm* 10 um; mobile phase: [water(FA)-ACN];gradient:10%-40% B over 10 min) to give Ex. 232 as a yellow solid.

Ex. 233 was prepared in the same manner as Ex. 188 with the appropriate linker.

Ex. 234 was prepared from Ex. 210 and 2-iodopropane following preparations described in General Method R.

### Preparation of (52R,E)-16-fluoro-71-(2-hydroxyethyl)-21,4-dimethyl-11H,21H,71H-3-oxa-1(5,3)-indazola-2(4,5),7(5,4)-dipyrazola-5(2,1)-pyrrolidinacyclononaphan-8-en-73-ol, I-235

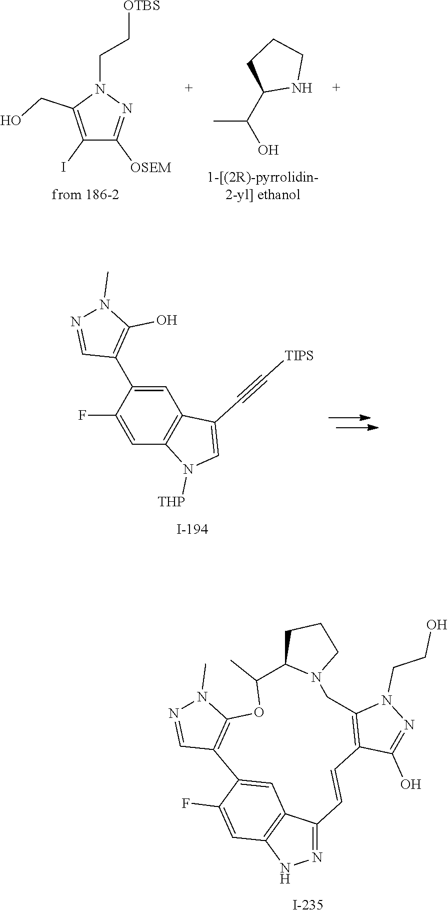

I-235 was synthesized following procedures described in General Method P and General M from the intermediates and starting material listed.

I-235 was combined with bromomethylcyclopropane following preparations described in General Method R to afford Ex. 235.

Ex. 236 was prepared in a similar manner to those described in General Method M with tert-butyl (S)-3-((methylsulfonyl)oxy)pyrrolidine-1-carboxylate as starting material and intermediates G-1-1, and I-156 where appropriate.

### Preparation of 4-[1-tetrahydropyran-2-yl-3-(2-triisopro pylsilylethynyl) indazol-5-yl]-1H-pyrazol-5-ol, I-237

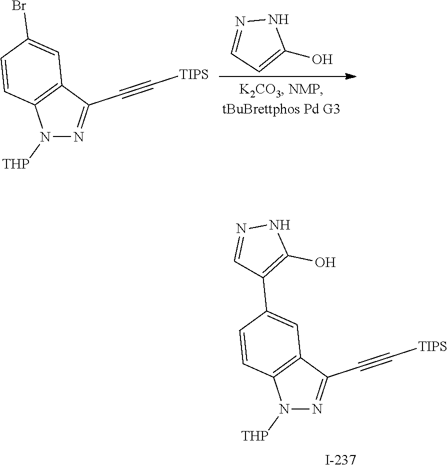

Step 1. The mixture of 1H-pyrazol-5-ol (109 mg, 1.30 mmol, 1.2 eq), 2-(5-bromo-1-tetrahydropyran-2-yl-indazol-3-yl)ethynyl-triisopropyl-silane (500 mg, 1.08 mmol, 1 eq.), tBuBrettphos (46.3 mg, 0.054 mmol, 0.05 eq), K₂CO₃ (299 mg, 2.17 mmol, 2 eq) in NMP (20 mL) was degassed and purged with N₂ for 3 times, and then the mixture was stirred at 130° C. for 2 h under N₂ atmosphere. On completion, the reaction mixture was quenched by addition H₂O 250 mL and extracted with EA (200 mL*3). The combined organic layers were washed with NaCl 300 mL, filtered and concentrated under reduced pressure to give a residue. The residue was purified by column chromatography (SiO₂, Petroleum ether/Ethyl acetate=0:1) to give 4-[1-tetrahydropyran-2-yl-3-(2-triisopro pylsilylethynyl) indazol-5-yl]-1H-pyrazol-5-ol (130 mg, 0.279 mmol, 26% yield) as a brown solid. ¹H NMR (400 MHz, DMSO-d₆) δ=12.00-11.46 (m, 1H), 10.48-9.89 (m, 1H), 8.02 (s, 1H), 7.87 (s, 1H), 7.71-7.59 (m, 2H), 3.82 (br d, J=11.2 Hz, 1H), 3.72-3.61 (m, 2H), 2.34-2.20 (m, 2H), 1.93-1.83 (m, 4H), 1.73-1.60 (m, 2H), 1.09-1.06 (m, 21H)

### Preparation of {(17E)-14-(2-hydroxyethyl)-12-(propan-2-yl)-16-[(propan-2-yl)oxy]-2,10,11,12,13,14-hexahydro-7H-3,5-ethenotripyrazolo[3,4-f:3′,4′-j:4″,3″-n][1,4]oxazacyclopentadecin-7-yl}acetonitrile (Ex. 237)

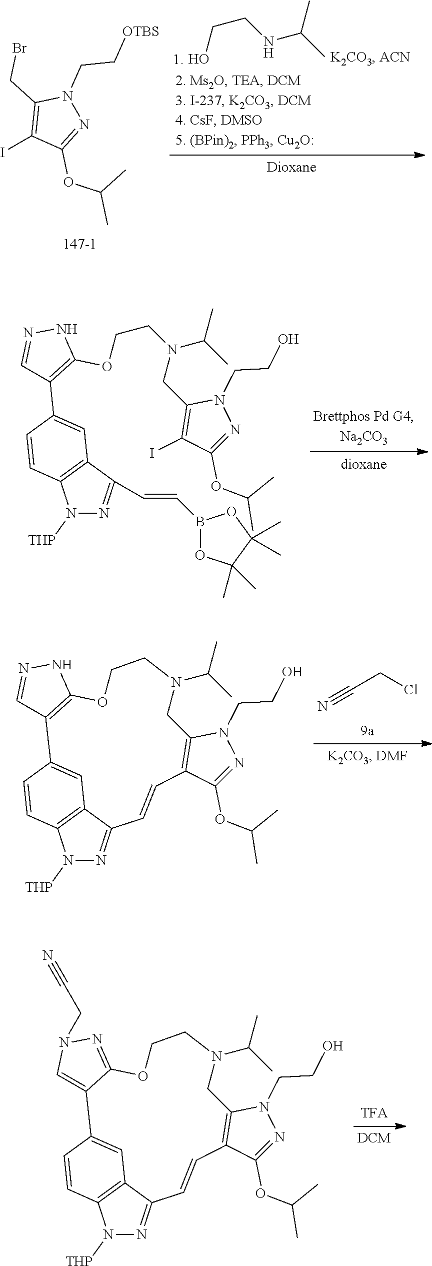

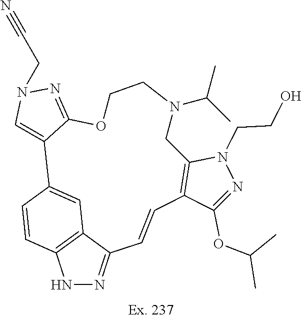 [SMILES: CC(C)Oc1nn(CCO)c2c1/C=C/c1n[nH]c3ccc(cc13)-c1cn(CC#N)nc1OCCN(C(C)C)C2]

Steps 1 through 5 were conducted in a manner similar to those described for General Method S.

Step 6 was conducted according to General Method Q.

Step 7. To a solution of 2-[(17E)-15-isopropoxy-10-isopropyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3, 12(16),14,17,19,22(26),23-nonaen-13-yl]ethanol (40.0 mg, 0.070 mmol, 1 eq), 2-chloroacetonitrile (10.4 mg, 0.138 mmol, 2 eq) in DMF (0.6 mL) was added K₂CO₃ (28.8 mg, 0.208 mmol, 3 eq). The mixture was stirred at 60° C. for 1 h. On completion, the mixture was diluted with water (2 mL) and extracted with ethyl acetate (1 mL×3). The combined organic phase was washed with water (2 mL*2) and dried over Na₂SO₄, filtered and the filtrate was concentrated to give a residue. The residue was purified by column chromatography (SiO₂, PE:THF=1:2) to give 2-[(17E)-13-(2-hydroxyethyl)-15-isopropoxy-10-isopropyl-21-tetrahydropyran-2-yl-7-oxa-4,5,10,13,14,20,21-hepta zapentacyclo[17.5.2.0^{2,6}.0^{12,16}0.0^{22,26}]hexacosa-1(25),2(6),3,12(16),14,17,19,22(26),23 -nonaen-5-yl]acetonitrile (12.0 mg, 0.020 mmol, 28% yield) as a yellow solid. LCMS: m/z 615.3 (M+t)

Step 8. To a solution of 2-[(17E)-13-(2-hydroxyethyl)-15-isopropoxy-10-isopropyl-21-tetrahydropyran-2-yl-7-oxa-4,5,10,13,14,20,21-heptazapentacyclo[17.5.2.0^{2,6}.0^{12,16}. 0^{22,26}]hexacosa-1(25),2,5,12(16),14,17,19,22(26),23-nonaen-4-yl]acetonitrile (9.00 mg, 0.015 mmol, 1 eq) in DCM (1 ml) was added TFA (460 mg, 4.04 mmol, 1 mL, 275 eq). The mixture was stirred at 25° C. for 1 h. On completion, the mixture was concentrated to give a residue. The residue was purified by prep-HPLC (neutral condition; column: Waters xbridge 150*25 mm 10 um; mobile phase: [water (NH₄HCO₃)-ACN];gradient:29%-59% B over 10 min) to Ex. 237 (1.76 mg, 0.003 mmol, 23% yield) as a white solid.

Ex. 238 was prepared in a similar manner to those described in General Method M with tert-butyl 3-hydroxyazetidine-1-carboxylate as starting material and intermediates G-1-1, and I-156 where appropriate.

### Preparation of 4-(6-fluoro-1-(tetrahydro-2H-pyran-2-yl)-3-vinyl-1H-indazol-5-yl)-1,3-dimethyl-1H-pyrazol-5-ol, 1-239

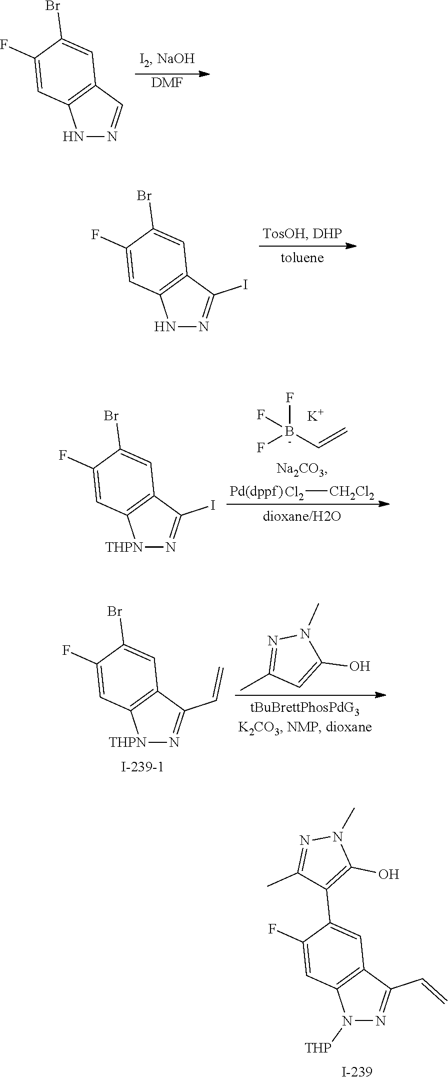

Steps 1 and 2 were performed in a similar manner to synthesis of C-1-1.

Step 3. To a solution of 5-bromo-6-fluoro-3-iodo-1-tetrahydropyran-2-yl-indazole (9.50 g, 22.4 mmol, 1 eq) in dioxane (100 mL) and H₂O (20 mL) was added potassium;trifluoro(vinyl)boranuide (2.99 g, 22.4 mmol, 1 eq), Na₂CO₃ (7.11 g, 67 mmol, 3 eq) and Pd(dppf)Cl₂·CH₂Cl₂ (1.83 g, 2.24 mmol, 0.1 eq) at 25° C. under N₂, then the mixture was stirred at 80° C. for 16 h. On completion, the reaction mixture was diluted with H₂O (150 mL) and extracted with EA (50 mL*3). The combined organic layers were washed with Brine (50 mL*2), dried over Na₂SO₄, filtered and concentrated under reduced pressure to give a residue. The residue was purified by flash silica gel chromatography (ISCO®; 120 g SepaFlash® Silica Flash Column, Eluent of 0-15% THE/Petroleum ether gradient @80 m/min). Compound 5-bromo-6-fluoro-1-tetrahydropyran-2-yl-3-vinyl-indazole (4.30 g, 13.2 mmol, 59% yield) as a yellow solid.

Step 4 was conducted in a manner similar to those described in I-237 to give I-239.

### General Method T: Preparation of 2-[(10S,17E)-16-ethoxy-19-fluoro-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 239)

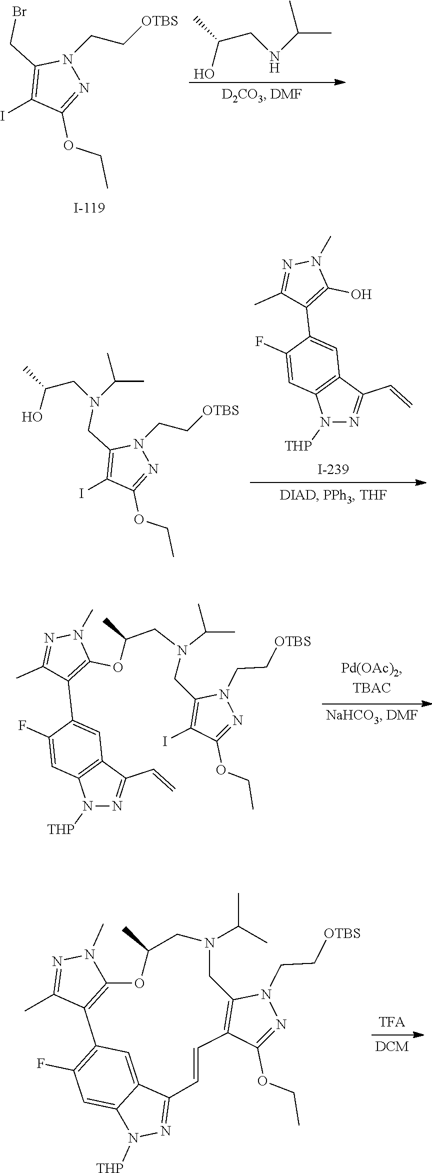

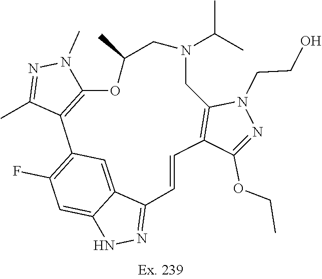 [SMILES: CCOc1nn(CCO)c2c1/C=C/c1n[nH]c3cc(F)c(cc13)-c1c(C)nn(C)c1O[C@@H](C)CN(C(C)C)C2]

Step 1. To a solution of 2-[5-(bromomethyl)-3-ethoxy-4-iodo-pyrazol-1-yl]ethoxy-tert-butyl-dimethyl-silane (1.00 g, 2.04 mmol, 1 eq) in DMF (10 mL) was added (2R)-1-(isopropylamino) propan-2-ol (240 mg, 2.04 mmol, 1 eq) and K₂CO₃ (847 mg, 6.13 mmol, 3 eq). The mixture was stirred at 80° C. for 2 h. On completion, the mixture was diluted with H₂O (20 mL) and extracted with EA (30 mL*3). The combined organic layers was dried over Na₂SO₄, filtered and the filtrate was concentrated under reduced pressure to give a residue. The residue was purified by column chromatography (SiO₂, Petroleum ether/Ethyl acetate=1/0 to 0/1) to give (2R)-1-[[2-[2-[tert-butyl (dimethyl) silyl]oxyethyl]-5-ethoxy-4-iodo-pyrazol-3-yl]methyl-isopropyl-amino]propan-2-ol (760 mg, 1.45 mmol, 71% yield) was obtained as yellow oil. LCMS: m/z 526.5 (M+1)

Step 2. A mixture of (2R)-1-[[2-[2-[tert-butyl(dimethyl)silyl]oxyethyl]-5-ethoxy-4-iodo-pyrazol-3-yl]methyl-isopropyl-amino]propan-2-ol (545 mg, 1.04 mmol, 1 eq), 4-(6-fluoro-1-tetrahydropyran-2-yl-3-vinyl-indazol-5-yl)-2,5-dimethyl-pyrazol-3-ol (443 mg, 1.24 mmol, 1.2 eq), PPh₃ (598 mg, 2.28 mmol, 2.2 eq) in THE (3 mL) was degassed and purged with N₂ for 3 times, and stirred at 25° C. for 0.5 h, and then DIAD (461 mg, 2.28 mmol, 2.2 eq) was added at 0° C., the mixture was stirred at 25° C. for 1.5 h under N₂ atmosphere. On completion, the reaction mixture was concentrated under reduced pressure to remove solvent. The residue was purified by column chromatography (SiO₂, Petroleum ether/Ethyl acetate=1/0 to 0/1) to give (2S)—N-[[2-[2-[tert-butyl(dimethyl)silyl]oxyethyl]-5-ethoxy-4-iodo-pyrazol-3-yl]methyl]-2-[4-(6-fluoro-1-tetrahydropyran-2-yl-3-vinyl-indazol-5-yl)-2,5-dimethyl-pyrazol-3-yl]oxy-N-isopropyl-propan-1-amine (800 mg, 0.926 mmol, 89% yield) as a yellow oil. LCMS: m/z 864.3 (M+1)

Step 3. A mixture of (2S)—N-[[2-[2-[tert-butyl(dimethyl)silyl]oxyethyl]-5-ethoxy-4-iodo-pyrazol-3-yl]methyl]-2-[4-(6-fluoro-1-tetrahydropyran-2-yl-3-vinyl-indazol-5-yl)-2,5-dimethyl-pyrazol-3-yl]oxy-N-isopropyl-propan-1-amine (501 mg, 0.580 mmol, 1 eq), TBAC (161 mg, 0.580 mmol, 1 eq), NaHCO₃ (122 mg, 1.45 mmol, 2.5 eq) and Pd(OAc)₂ (26.1 mg, 0.116 mmol, 0.2 eq) in DMF (6 mL) was degassed and purged with N₂ for 3 times, and then the mixture was stirred at 140° C. for 1 h under N₂ atmosphere. On completion, the reaction mixture was diluted with H₂O (30 mL) and extracted with EA (30 mL*3). The combined organic layers were washed with H₂O (20 mL*3), dried over Na₂SO₄, filtered and concentrated under reduced pressure to give a residue. The residue was purified by column chromatography (SiO₂, Petroleum ether/THF=1/0 to 0/1) to give tert-butyl-[2-[(8S,17E)-15-ethoxy-24-fluoro-10-isopropyl-3, 5,8-trimethyl-21-tetrahydropyran-2-yl-7-oxa-4,5,10,13,14,20,21-heptazapentacyclo [17.5.2.02,6.012,16.022,26]hexacosa-1(24),2(6),3,12(16),14,17,19,22,25-nonaen-13-yl]ethoxy]-dimethyl-silane (360 mg, 0.489 mmol, 84% yield) as a brown oil. LCMS: m/z 736.3 (M+1)

Step 4. To a solution of tert-butyl-[2-[(8S,17E)-15-ethoxy-24-fluoro-10-isopropyl-3,5,8-trimethyl-21-tetrahydropyran-2-yl-7-oxa-4,5,10,13,14,20,21-heptazapentacyclo[17.5.2.02,6.012,16.022,26]hexacosa-1(24),2(6),3,12(16),14,17,19,22,25-nonaen-13-yl]ethoxy]-dimethyl-silane (130 mg, 0.177 mmol, 1 eq) in DCM (1.5 mL) was added TFA (2.30 g, 20.2 mmol, 1.5 mL, 114 eq). The mixture was stirred at 25° C. for 1 h. On completion, the reaction mixture was concentrated under reduced pressure to remove solvent. The residue was purified by Prep-HPLC (column: Phenomenex luna C18 150*25 mm*10 um; mobile phase: [water (FA)-ACN]; gradient: 12%-42% B over 10 min) to give Ex. 239 (16.7 mg, 0.028 mmol, 16% yield, 96.50% purity, FA) as a white solid.

### Preparation of (10S,18E)-17-ethoxy-8,10,12-trimethyl-2,8,10,11,12,13-hexahydro-3,5-ethenodipyrazolo[3,4-j:4′,3′-n]pyrido[4,3-f][1,4]oxazacyclopentadecine, Ex. 240

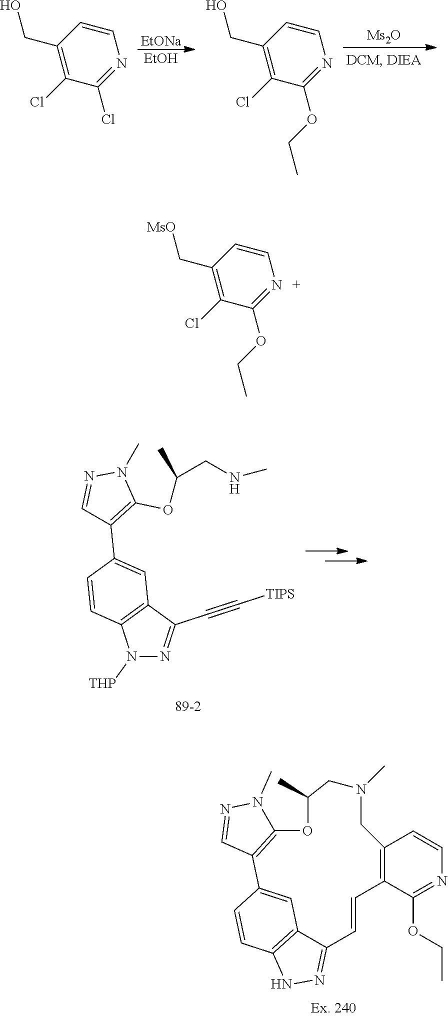

Step 1. Freshly prepared EtONa in EtOH (from Na (4.84 g, 210 mmol, 12.5 eq) in dry EtOH (25 mL)) was added dropwise to a solution of (2,3-dichloro-4-pyridyl)methanol (3.00 g, 16.8 mmol, 1 eq) in EtOH (40 mL). The mixture was stirred at 80° C. for 16 h. LCMS showed part of the reactant 1 was remained and one peak with desired mass was detected. Then freshly prepared EtONa in EtOH (from Na (4.84 g, 210 mmol, 12.5 eq) in dry EtOH (25 mL)) was added to the reaction solution. On completion, the mixture was concentrated under reduced pressure to give a residue. The residue was quenched with H₂O (300 mL) and extracted with EtOAc (3*300 mL). The combined organic layers were washed with brine (3*200 mL), dried over Na₂SO₄ and concentrated in vacuum. The residue was purified by column chromatography (SiO₂, Petroleum ether/Ethyl acetate=1/0 to 1/1) to give (3-chloro-2-ethoxy-4-pyridyl)methanol (1.48 g, 6.27 mmol, 37% yield, 79% purity) as a yellow solid. ¹H NMR (400 MHz, DMSO-d₆) δ=8.09 (d, J=5.2 Hz, 1H), 7.16 (d, J=5.2 Hz, 1H), 5.59 (t, J=5.6 Hz, 1H), 4.56 (d, J=5.6 Hz, 2H), 4.38 (q, J=7.2 Hz, 2H), 1.33 (t, J=7.2 Hz, 3H). LCMS: m/z 188.0 (M+1).

Step 2. To a solution of (3-chloro-2-ethoxy-4-pyridyl)methanol (1.45 g, 7.73 mmol, 1 eq), DIEA (3.00 g, 23.1 mmol, 4.04 mL, 3 eq) in DCM (30 mL) at 0° C. was added methylsulfonylmethanesulfonate (2.02 g, 11.5 mmol, 1.5 eq). The mixture was stirred at 25° C. for 16 h. On completion, the mixture was concentrated under reduced pressure to give (3-chloro-2-ethoxy-4-pyridyl)methyl methanesulfonate (2.05 g, 7.72 mmol, 100% yield, crude) as a yellow solid. LCMS: m/z 265.9 (M+1). [01838]3-chloro-2-ethoxy-4-pyridyl)methyl methanesulfonate was combined with 89-2 in a manner similar to those described in Ex. 191 to afford Ex. 240 as a white solid.

### Preparation of 1-((R)-pyrrolidin-2-yl)ethan-1-ol, 1-241

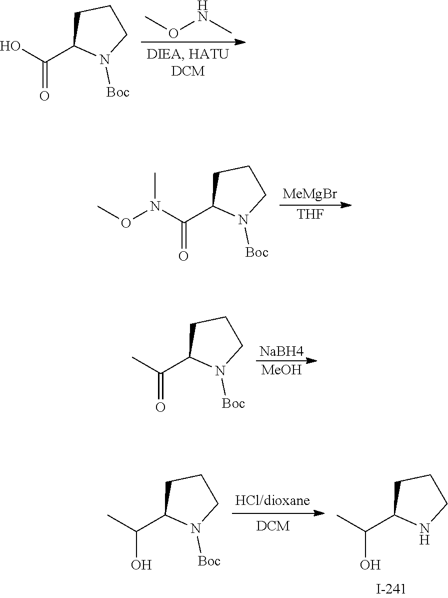

Step 1. To a solution of (2R)-1-tert-butoxycarbonylpyrrolidine-2-carboxylic acid (20.0 g, 92.9 mmol, 1 eq), N-methoxymethanamine (6.24 g, 102 mmol, 1.10 eq), DIEA (24.0 g, 185 mmol, 2.00 eq), HATU (38.8 g, 102 mmol, 1.10 eq) in DCM (200 mL). The mixture was stirred at 25° C. for 1 hr. On completion, the mixture was diluted with water (300 mL) and extracted with DCM (60 mL×3). The combined organic phase was washed with water (200 mL*2) and dried over Na₂SO₄, filtered and the filtrate was concentrated to give a residue. The residue was purified by column chromatography (SiO₂, DCM/MeOH=1:0 to 90:10) to give tert-butyl (R)-2-(methoxy(methyl)carbamoyl)pyrrolidine-1-carboxylate (11.0 g, 42.5 mmol, 46% yield) as a yellow oil. LCMS: m/z 159.1 (M+1-100).

Step 2. To a solution of tert-butyl (2R)-2-[methoxy(methyl)carbamoyl]pyrrolidine-1-carboxylate (10.0 g, 38.7 mmol, 1.00 eq) in THF (100 mL) was added bromo(methyl)magnesium (3 M, 38.7 mL, 3.00 eq) at −78° C. under atmosphere. The mixture was stirred at −78° C. for 2 hr. On completion, the reaction mixture was quenched with sat. NH₄Cl (200 mL) at 0° C., and then diluted with H₂O (150 mL) and extracted with EA (60 mL*3). The combined organic layers were dried over Na₂SO₄, filtered and concentrated under reduced pressure to give a residue. The residue was purified by column chromatography (SiO₂, Petroleum ether/Ethyl acetate=1:0 to 1:1) to give tert-butyl (R)-2-acetylpyrrolidine-1-carboxylate (7.00 g, 32.8 mmol, 85% yield) as a yellow oil. ¹H NMR (400 MHz, CDCl₃) δ=4.37-4.13 (m, 1H), 3.60-3.38 (m, 2H), 2.31-2.00 (m, 4H), 1.92-1.76 (m, 3H), 1.52-1.37 (m, 9H).

Step 3. To a solution of tert-butyl (2R)-2-acetylpyrrolidine-1-carboxylate (6.50 g, 30.4 mmol, 1.00 eq) in MeOH (65 mL) was added NaBH₄ (3.46 g, 91.4 mmol, 3.00 eq) at 0° C. The mixture was stirred at 0° C. for 2 hr. On completion, the mixture was concentrated to give a residue. The residue was purified by column chromatography (SiO₂, Petroleum ether/Ethyl acetate=1:0 to 1:1) to give tert-butyl (2R)-2-(1-hydroxyethyl)pyrrolidine-1-carboxylate (5.60 g, 26.0 mmol, 85% yield) as a yellow oil. ¹H NMR (400 MHz, CDCl₃) δ=4.01-3.81 (m, 1H), 3.77-3.45 (m, 2H), 3.31-3.20 (m, 1H), 2.05-1.64 (m, 4H), 1.46 (s, 9H), 1.16-1.03 (m, 3H). LCMS: m/z 116.2 (M+1-100)

Step 4. To a solution of tert-butyl (2R)-2-(1-hydroxyethyl)pyrrolidine-1-carboxylate (1.00 g, 4.64 mmol, 1.00 eq) in DCM (10 mL) was added HCl/dioxane (4 M, 2.00 mL, 1.72 eq). The mixture was stirred at 25° C. for 2 hr. On completion, the mixture was concentrated to give 1-((R)-pyrrolidin-2-yl)ethan-1-ol, I-241 (700 mg, crude) as a white oil. [01844]1-241 was combined with 119-2, then 188-1 following synthesis described in General Method S to give 3 compounds after prep HPLC: arbitrarily assigned stereo isomers Ex. 241, Ex. 242, and rearranged by product Ex. 243

I-241 and 119-2 were combined with 1-194 in the same way to afford arbitrarily assigned Ex. 243, ex. 244, and rearranged by product Ex. 245.

### Preparation of (2R)-1-[[2-[2-[tert-butyl(dimethyl)silyl]oxyethyl]-4-iodo-5-isopropoxy-pyrazol-3-yl]methyl-methylamino]propan-2-ol (1-246)

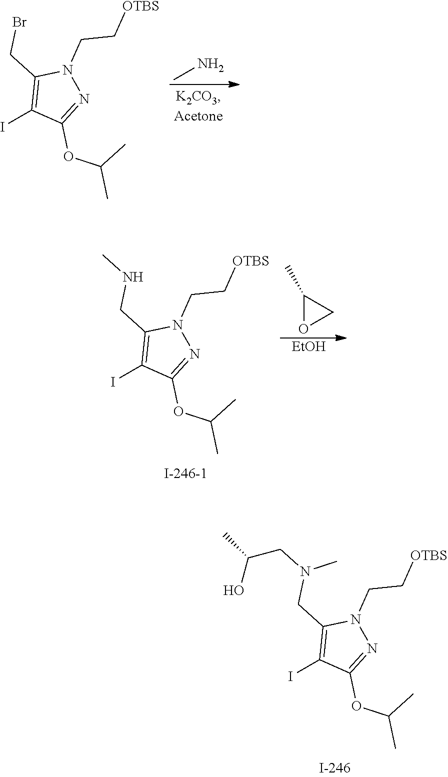

Step 1. To a solution of 2-[5-(bromomethyl)-4-iodo-3-isopropoxy-pyrazol-1-yl]ethoxy-tert-butyl-dimethyl-silane (2.00 g, 3.97 mmol, 1 eq) in acetone (50 mL) was added methanamine (2 M, 19.9 mL, 10 eq) and K₂CO₃ (1.65 g, 11.9 mmol, 3 eq). The mixture was stirred at 20° C. for 1 hour. On completion, the mixture was filtered and concentrated to give the crude compound. The residue was purified by column chromatography (SiO₂, Petroleum ether/Ethyl acetate=10/1 to 2/1) to give 1-[2-[2-[tert-butyl(dimethyl)silyl]oxyethyl]-4-iodo-5-isopropoxy-pyrazol-3-yl]-N-methyl-methanamine (800 mg, 1.76 mmol, 44% yield) as a brown oil. LCMS: m/z 454.3 (M+1).

Step 2. To a solution of 1-[2-[2-[tert-butyl(dimethyl)silyl]oxyethyl]-4-iodo-5-isopropoxy-pyrazol-3-yl]-N-methylmethanamine (800 mg, 1.76 mmol, 1 eq) in EtOH (5 ml) was added (2R)-2-methyloxirane (123 mg, 2.12 mmol, 1.2 eq). The mixture was concentrated to give (2R)-1-[[2-[2-[tert-butyl(dimethyl)silyl]oxyethyl]-4-iodo-5-isopropoxy-pyrazol-3-yl]methyl-methylamino]propan-2-ol (520 mg, 1.02 mmol, 58% yield) as a brown oil. LCMS: m/z 512.0 (M+1).

### Preparation of 2-{(10S,17E)-8-cyclopropyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 246)

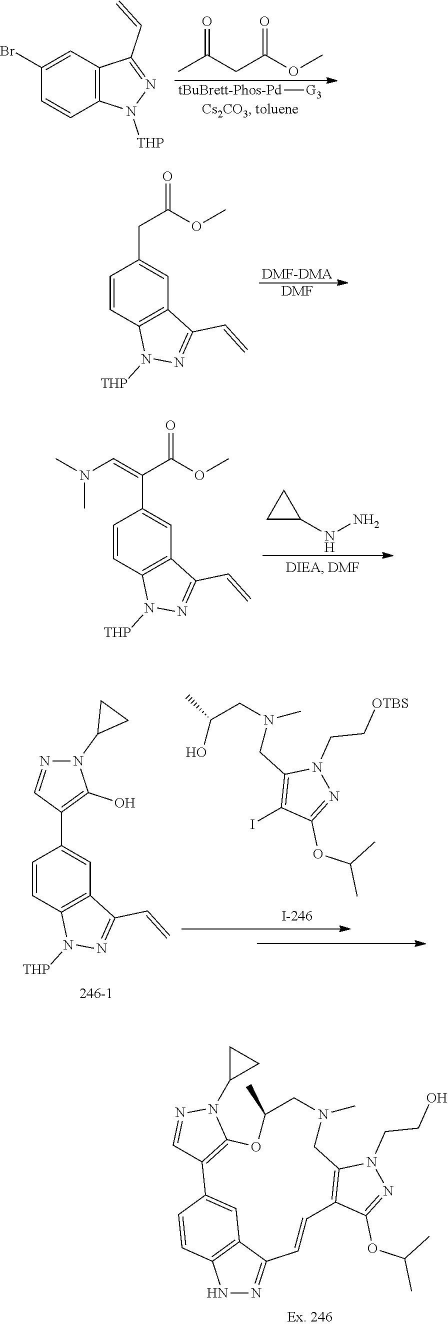

Step 1. A mixture of 5-bromo-1-tetrahydropyran-2-yl-3-vinyl-indazole (5.00 g, 16.3 mmol, 1 eq), methyl 3-oxobutanoate (2.83 g, 24.4 mmol, 1.5 eq), [2-(2-aminophenyl)phenyl]-methylsulfonyloxy-palladium;ditert-butyl-[3,6- dimethoxy-2-(2,4,6-triisopropylphenyl)phenyl]phosphane (695 mg, 0.814 mmol, 0.05 eq), Cs₂CO₃ (15.9 g, 48.8 mmol, 3 eq) in toluene. (60 mL) was degassed and purged with N₂ for 3 times, and then the mixture was stirred at 70° C. for 12 hours under N₂ atmosphere. On completion, the reaction mixture was concentrated in vacuum. The residue was purified by silica gel chromatography (80 g silica gel column, THF in PE from 0% to 10%) to give methyl 2-(1-tetrahydropyran-2-yl-3-vinyl-indazol-5-yl)acetate (3.10 g, 10.3 mmol, 63% yield) as a brown oil.

Step 2. To a solution of methyl 2-(1-tetrahydropyran-2-yl-3-vinyl-indazol-5-yl)acetate (3.10 g, 10.3 mmol, 1 eq) in DMF (20 mL) was added DMF-DMA (1.84 g, 15.5 mmol, 1.5 eq). The mixture was stirred at 60° C. for 16 hours. On completion, the reaction mixture was concentrated in vacuum to give methyl (E)-3-(dimethylamino)-2-(1-tetrahydropyran-2-yl-3-vinyl-indazol-5-yl)prop-2-enoate (3.60 g, 10.1 mmol, 98% yield) as a brown oil. LCMS: m/z 356.2 (M+1).

Step 3. To a solution of methyl (E)-3-(dimethylamino)-2-(1-tetrahydropyran-2-yl-3-vinyl-indazol-5-yl)prop-2-enoate (3.60 g, 10.1 mmol, 1 eq), cyclopropylhydrazine;hydrochloride (1.65 g, 15.2 mmol, 1.5 eq) in DMF (30 mL) was added DIEA (6.55 g, 50.6 mmol, 5 eq). The mixture was stirred at 60° C. for 1 hour. On completion, the reaction mixture was poured into water (100 mL), extracted with ethyl acetate (50 mL * 3), the combined organic layers were washed with brine (40 mL*2), dried over Na₂SO₄, filtered and concentrated under reduced pressure to give a residue. The residue was purified by column chromatography (SiO₂, Petroleum ether/Ethyl acetate=10/1 to 0/1) to give 2-cyclopropyl-4-(1-tetrahydropyran-2-yl-3-vinyl-indazol-5-yl)pyrazol-3-ol (1.80 g, 5.14 mmol, 51% yield) as a brown oil. LCMS: m/z 351.2 (M+1).

The remaining steps were conducted according to General Method T from 246-1 and 1-246 to afford Ex. 246.

### Preparation of (10S,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine, (10R,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine, (10R,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine, and (10S,13R,19E)-18-ethoxy-8,13,16-trimethyl-2,8,10,11,12.13,15,16-octahydro-3,5-etheno-10,14-methanotripyrazolo[3,4-h:3′,4′-l:4″,3″-p][1,6]oxazacycloheptadecine (Ex. 247-250)

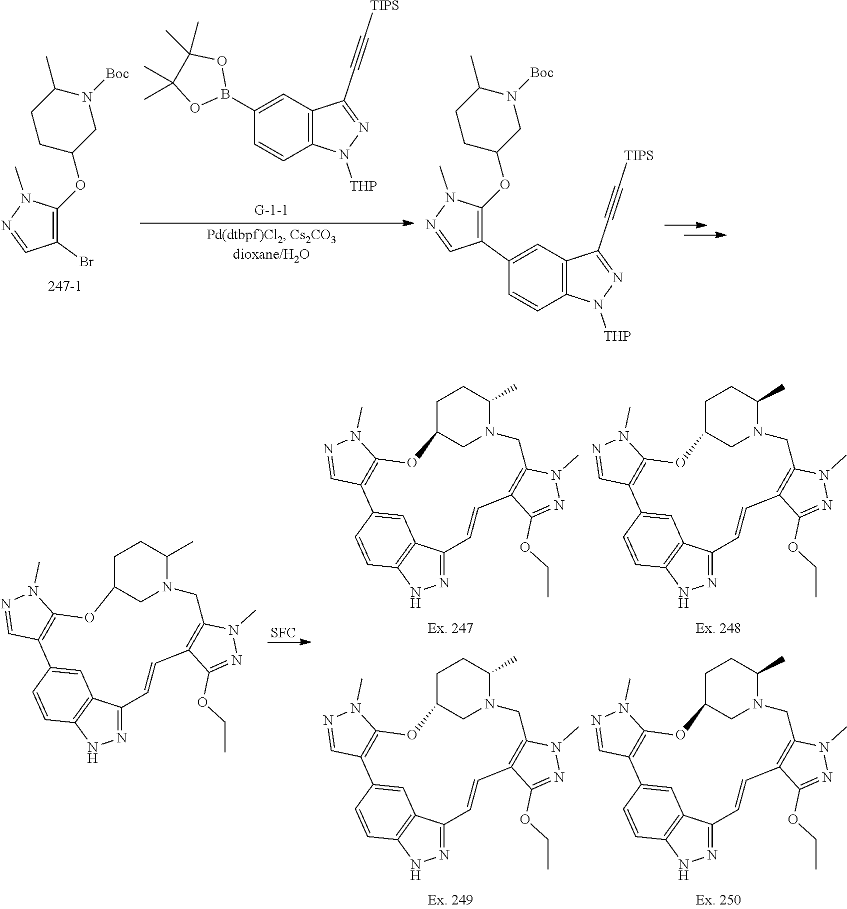

Step 1. A mixture of tert-butyl 5-(4-bromo-2-methyl-pyrazol-3-yl)oxy-2-methyl-piperidine-1-carboxylate (1.5 g, 4.01 mmol, 1 eq, from General Method M), triisopropyl-[2-[1-tetrahydropyran-2-yl-5-(4,4,5,5-tetramethyl-1,3,2-dioxaborolan-2-yl) indazol-3-yl]ethynyl]silane (1.63 g, 3.21 mmol, 0.8 eq), ditert-butyl(cyclopentyl)phosphane; dichloropalladium;iron (130 mg, 0.200 mmol, 0.05 eq), Cs₂CO₃ (1.31 g, 4.01 mmol, 1 eq) in dioxane (30 mL) and H₂O (6 mL) was degassed and purged with N₂ for 3 times, and then the mixture was stirred at 80° C. for 12 hours under N₂ atmosphere. On completion the reaction mixture was quenched by addition H₂O (100 mL), and extracted with EA (100 mL*3). The combined organic layers were washed with sat. NaCl (100 mL), concentrated under reduced pressure to give a residue. The residue was purified by column chromatography (SiO₂, Petroleum ether/Ethyl acetate=1:1) to give tert-butyl 2-methyl-5-[2-methyl-4-[1-tetrahydropyran-2-yl-3-(2-triisopropylsilylethynyl) indazol-5-yl]pyrazol-3-yl]oxy-piperidine-1-carboxylate (2 g, 2.96 mmol, 73% yield) as a white solid. LCMS: m/z 676.4 (M+1)

tert-butyl 2-methyl-5-[2-methyl-4-[1-tetrahydropyran-2-yl-3-(2-triisopropylsilylethynyl) indazol-5-yl]pyrazol-3-yl]oxy-piperidine-1-carboxylate was converted to mixtures of isomers: tert-butyl 2-methyl-5-[2-methyl-4-[1-tetrahydropyran-2-yl-3-(2-triisopropylsilylethynyl) indazol-5-yl]pyrazol-3-yl]oxy-piperidine-1-carboxylate via General Method M.

SFC separation resulted in 4 compounds that were arbitrarily assigned as Ex. 247, Ex. 248, Ex. 249, and Ex. 250.

### Preparation of 4-(6-fluoro-1-tetrahydropyran-2-yl-3-vinyl-indazol-5-yl)-2-methyl-pyrazol-3-ol (I-251)

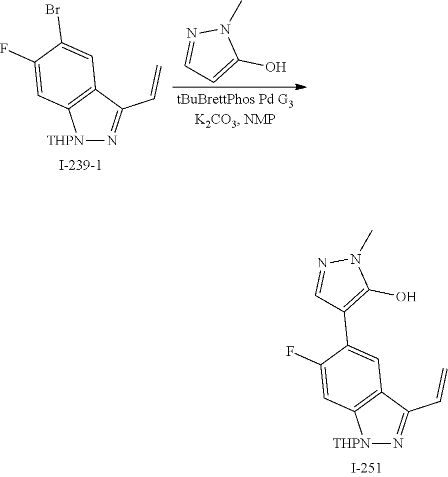

I-239-1 was converted to 1-251 following procedure detailed in the synthesis of 1-237.

Ex. 251 was isolated as a byproduct from a mixture obtained from 1-251 and 1-246 following General Method T.

### Preparation of I-252

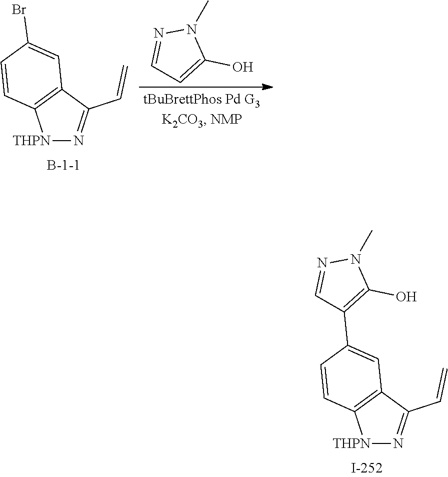

I-252 was prepared according to synthesis of I-237 from B-1-1.

### General Method U: Preparation of 2-[(8aR,9S,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol (Ex. 252), 2-[(8aR,9R,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol (Ex. 253), and 2-[(19E)-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol (Ex. 254)

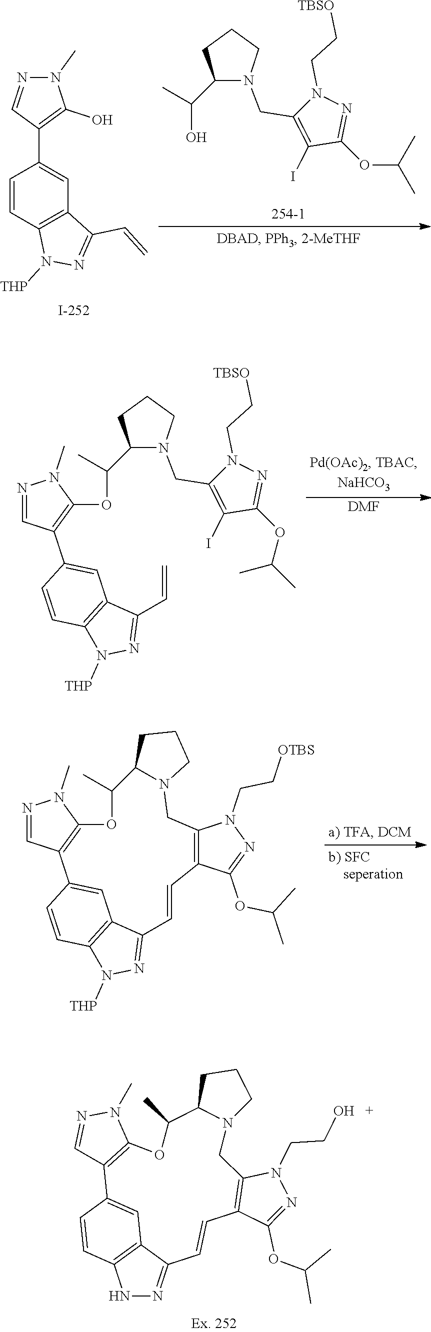

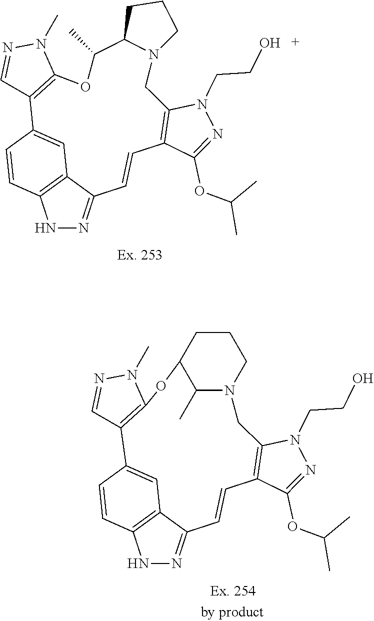

254-1 was synthesized from I-241 and 147-1 following General Method T.

Step 1. A mixture of 1-[(2R)-1-[[2-[2-[tert-butyl(dimethyl)silyl]oxyethyl]-4-iodo-5-isopropoxy-pyrazol-3-yl]methyl]pyrrolidin-2-yl]ethanol (1.00 g, 1.86 mmol, 1 eq), 2-methyl-4-(1-tetrahydropyran-2-yl-3-vinyl-indazol-5-yl)pyrazol-3-ol (965 mg, 2.98 mmol, 1.6), DBAD (942 mg, 4.09 mmol, 2.2 eq), PPh₃ (1.07 g, 4.09 mmol, 2.2 eq) was degassed and purged with N₂ for 3 times, then 2-methyltetrahydrofuran (10 mL) was degassed and purged with N₂ for 3 times again, and then the mixture was stirred at 25° C. for 2 h under N₂ atmosphere. On completion, the mixture was concentrated in vacuo to give a residue. The residue was purified by column chromatography (SiO₂, Petroleum ether/THF=3:1 to 1:2) to give tert-butyl-[2-[4-iodo-3-isopropoxy-5-[[(2R)-2-[1-[2-methyl-4-(1-tetrahydropyran-2-yl-3-vinyl-indazol-5-yl)pyrazol-3-yl]oxyethyl]pyrrolidin-1-yl]methyl]pyrazol-1-yl]ethoxy]-dimethyl-silane (1.20 g, 1.42 mmol, 76% yield) as white solid. LCMS: m/z 844.3 (M+1).

Steps 2 and 3 were performed in a similar manner to General Method T followed by SFC separation to give Ex. 252, Ex. 253 and by product 254. Absolute stereochemistry of 253 was determined by X-ray analysis.

### Preparation of 2-[(8aR,9R,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol (Ex. 255), 2-[(8aR,9S,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol (Ex. 256), and 2-[(19E)-22-fluoro-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol (Ex.257)

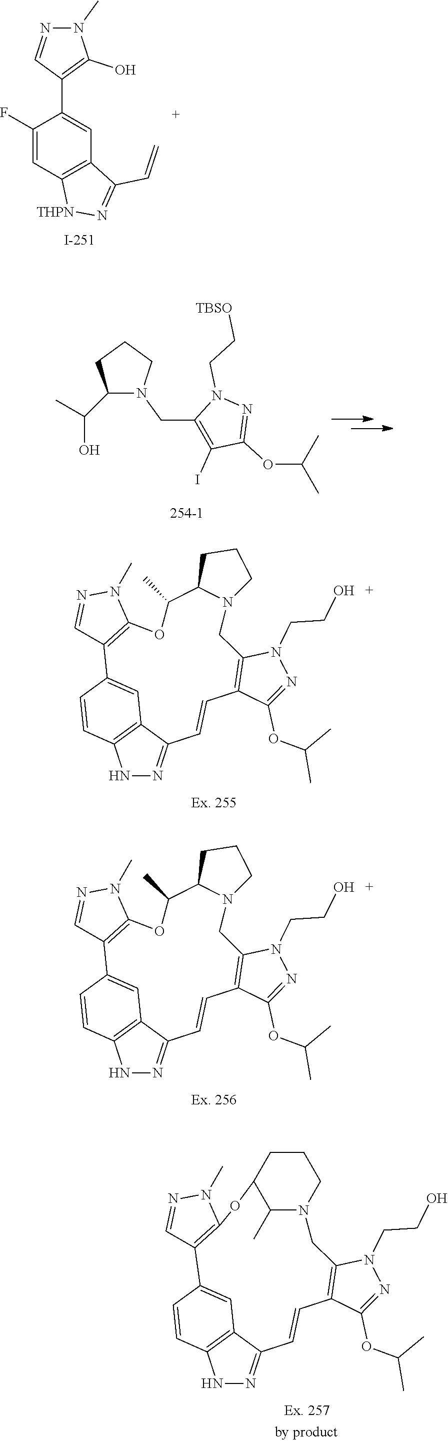

Examples 255, 256 and 257 were synthesized using the procedure of General Method U starting with I-251 and 254-1. SFC separation was used to obtain arbitrarily assigned 255 and 256 as well as by product 257.

### Preparation of 2-{(10R,17E)-10-(hydroxymethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 258)

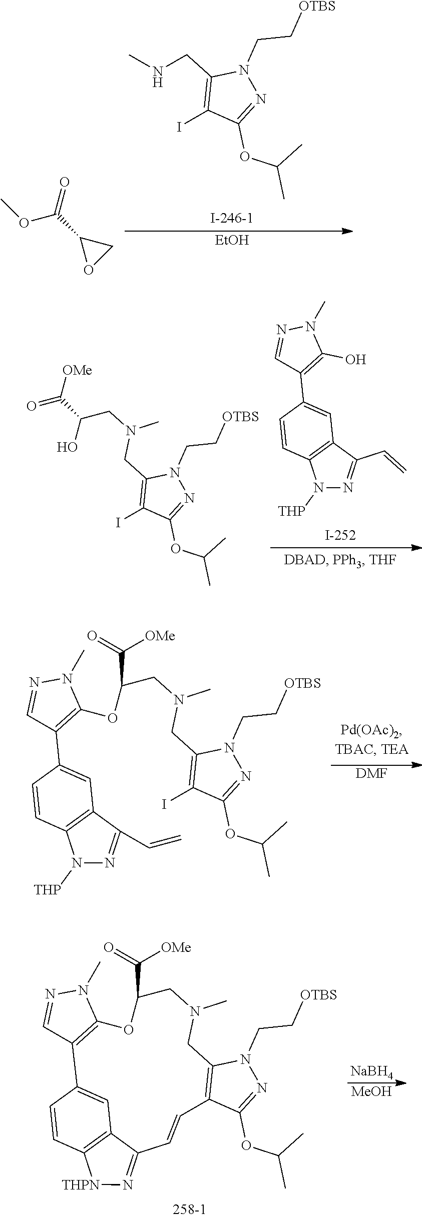

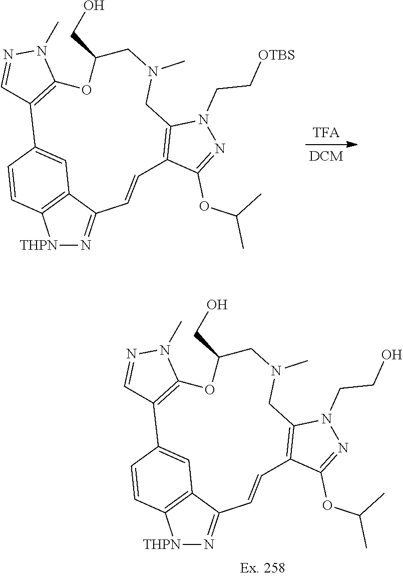

Step 1. To a solution of methyl (2S)-oxirane-2-carboxylate (1.01 g, 9.92 mmol, 3 eq) 1-[2-[2-[tert-butyl(dimethyl)silyl]oxyethyl]-4-iodo-5-isopropoxy-pyrazol-3-yl]-N-methyl-methanamine (1.50 g, 3.31 mmol, 1 eq) in EtOH (15 mL). The mixture was stirred at 80° C. for 2 h. On completion. The mixture was filtered and concentrated to give methyl (2R)-3-[[2-[2-[tert-butyl(dimethyl)silyl]oxyethyl]-4-iodo-5-isopropoxy-pyrazol-3-yl]methyl-methyl-amino]-2-hydroxy-propanoate (2.00 g, 3.24 mmol, 98% yield, 90% purity) as a yellow oil. LCMS: m/z 557.0 (M+1).

Step 2. To a solution of methyl (2S)-3-[[2-[2-[tert-butyl(dimethyl)silyl]oxyethyl]-4-iodo-5-isopropoxy-pyrazol-3-yl]methyl-methyl-amino]-2-hydroxy-propanoate (1.90 g, 3.42 mmol, 1 eq), 2-methyl-4-(1-tetrahydropyran-2-yl-3-vinyl-indazol-5-yl)pyrazol-3-ol (1.66 g, 5.13 mmol, 1.5) in THF (50 mL) was added PPh₃ (1.97 g, 7.52 mmol, 2.2 eq) and DBAD (1.73 g, 7.52 mmol, 2.2 eq). The mixture was stirred at 25° C. for 2 h. On completion, the mixture was concentrated to give a residue. The residue was purified by column chromatography (SiO₂, PE/THF=2:1 to 1:1) to give methyl (2R)-3-[[2-[2-[tert-butyl(dimethyl)silyl]oxyethyl]-4-iodo-5-isopropoxy-pyrazol-3-yl]methyl-methyl-amino]-2-[2-methyl-4-(1-tetrahydropyran-2-yl-3-vinyl-indazol-5-yl)pyrazol-3-yl]oxy-propanoate (2.5F0 g, 2.90 mmol, 85% yield) as a yellow solid. LCMS: m/z 862.2 (M+1).

Step 3. To a solution of methyl (2R)-3-[[2-[2-[tert-butyl(dimethyl)silyl]oxyethyl]-4-iodo-5-isopropoxy-pyrazol-3-yl]methyl-methyl-amino]-2-[2-methyl-4-(1-tetrahydropyran-2-yl-3-vinyl-indazol-5-yl)pyrazol-3-yl]oxy-propanoate (2.50 g, 2.90 mmol, 1 eq), TEA (1.17 g, 11.6 mmol, 4 eq) in DMF (250 mL) was added Pd(OAc)₂ (97.7 mg, 0.435 mmol, 0.15 eq) and TBAC (806 mg, 2.90 mmol, 1 eq). The mixture was stirred at 100° C. for 2 h. On completion, the mixture was quenched with water (200 mL) and extracted with ethyl acetate (250 mL×3), the combined organic phase was dried over anhydrous sodium sulfate, filtered and concentrated to give a residue. The residue was purified by column chromatography (SiO₂, PE/EA=1:1 to 0:1) to give methyl (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo[17.5.2.02,6.012,16.022,26]hexacosa-1(25),2(6),3,12(16),14,17,19,22(26),23-nonaene-8-carboxylate (1.00 g, 1.36 mmol, 47% yield) as a white solid. LCMS: m/z 736.3 (M+1).

Step 4. To a solution of methyl (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptaza pentacyclo[17.5.2.0^{2,6}.0^{12,16}0.0^{22,26}]hexacosa-1(25),2(6),3,12(16),14,17,19,22(26),23-nonaene-8-carboxylate (200 mg, 0.272 mmol, 1 eq) in MeOH (1 mL) was added NaBH₄ (103 mg, 2.72 mmol, 10 eq). The mixture was stirred at 0° C. for 2 h. On completion, the mixture was quenched with water (20 mL) and extracted with ethyl acetate (25 mL×3), the combined organic phase was dried over anhydrous sodium sulfate, filtered and concentrated to give [(8R,17E)-13-[2-[tert-butyl(dimethyl)silyl]oxyethyl]-15-isopropoxy-5,10-dimethyl-21-tetrahydropyran-2-yl-7-oxa-4,5,10,13,14, 20,21-heptazapentacyclo[17.5.2.02,6.012,16.022,26]hexacosa-1(25),2(6),3,12(16),14, 17,19,22(26),23-nonaen-8-yl]methanol (190 mg, 0.269 mmol, 98% yield) as a white solid. LCMS: m/z 706.3 (M+1).

Step 5. To a solution of [(8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo [17.5.2.0^{2,6}.0^{12,16}.0^{22,26}]hexacosa-1(25),2(6),3,12(16),14,17,19,22(26),23-nonaen-8-yl]methanol (120 mg, 0.169 mmol, 1 eq) in DCM (2 mL) was added TFA (3.07 g, 26.9 mmol, 2 mL, 158 eq).The mixture was stirred at 25° C. for 1 hr. On completion, the mixture was concentrated to give a residue. The residue was purified by column chromatography (SiO₂, DCM/MeOH=30:1 to 15:1) to give 2-{(10R,17E)-10-(hydroxymethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (31.31 mg, 0.062 mmol, 36% yield) as a white solid.

### General Method V: Preparation of 2-[(11S,17E)-16-ethoxy-11-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 259), and 2-[(10S,17E)-16-ethoxy-10-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 260)

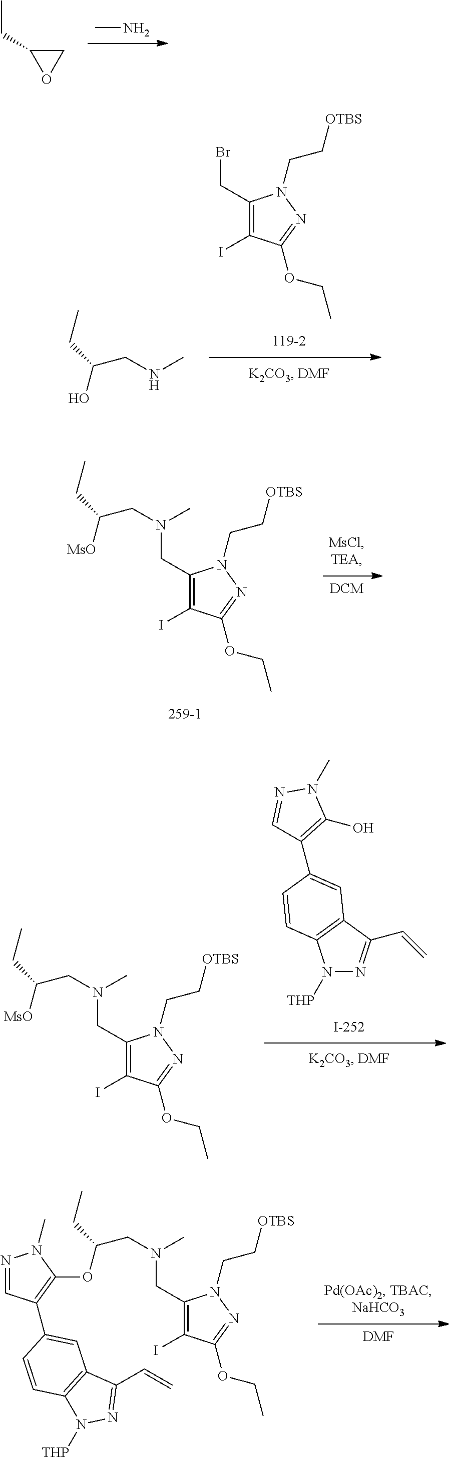

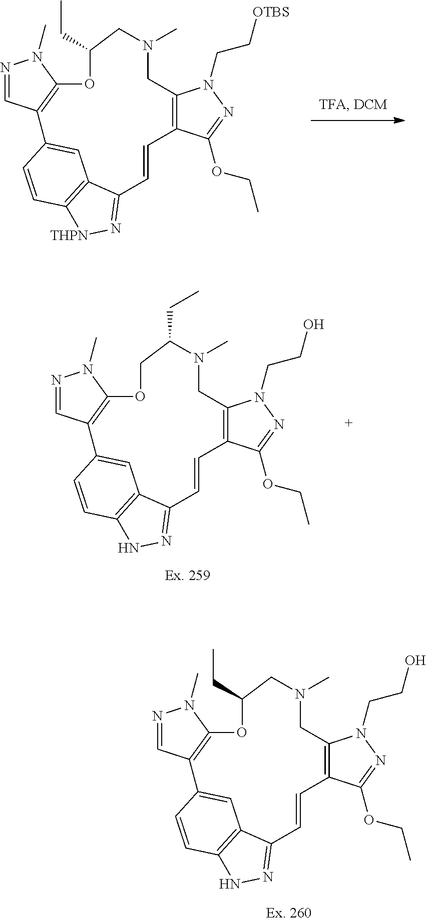

Step 1. To a solution of (2R)-2-ethyloxirane (2.00 g, 27.7 mmol, 1 eq) was added MeNH₂ (23.6 mL, 2.13 eq). The mixture was stirred at 25° C. for 12 hours. On completion, the reaction mixture was quenched by addition H₂O (30 mL) and extracted with EA (30 mL*3) to give the compound (2R)-1-(methylamino)butan-2-ol (2 g, 19.3 mmol, 69% yield) as a yellow solid. ¹H NMR (400 MHz, CDCl₃) δ=3.61-3.44 (m, 1H), 2.90-2.51 (m, 2H), 2.39-2.26 (m, 3H), 2.26-2.21 (m, 1H), 1.43-1.33 (m, 2H), 0.90 (t, J=7.2 Hz, 3H).

Step 2. To a solution of (2R)-1-(methylamino)butan-2-ol (1.00 g, 9.69 mmol, 1 eq) 2-[5-(bromomethyl)-3-ethoxy-4-iodo-pyrazol-1-yl]ethoxy-tert-butyl-dimethyl-silane (4.74 g, 9.69 mmol, 1 eq) in DMF (30 mL) was added K₂CO₃ (4.02 g, 29 mmol, 3 eq). The mixture was stirred at 80° C. for 1 hour. On completion, the reaction mixture was quenched by addition H₂O (100 mL) and extracted with EA (80 mL*3). The combined organic layers were washed with NaCl (100 ML), filtered and concentrated under reduced pressure. The residue was purified by column chromatography (SiO₂, Petroleum ether/Ethyl acetate=3:1) to give the compound (2R)-1-[[2-[2-[tert-butyl(dimethyl)silyl]oxyethyl]-5-ethoxy-4-iodo-pyrazol-3-yl]methyl-methyl-amino]butan-2-ol (600 mg, 1.17 mmol, 12% yield) as a white solid. LCMS: m/z 512.1 (M+1).

Step 3. To a solution of (2R)-1-[[2-[2-[tert-butyl(dimethyl)silyl]oxyethyl]-5-ethoxy-4-iodo-pyrazol-3-yl]methyl-methyl-amino]butan-2-ol (400 mg, 0.782 mmol, 1 eq) in DCM (8 mL) was added MsCl (1.17 mmol, 90 μL, 1.5 eq) and TEA (2.35 mmol, 350 μL, 3 eq). The mixture was stirred at 25° C. for 1 hour. On completion, the mixture was quenched with sat. NaHCO₃ (25 mL). The mixture was separated and the aqueous layer was extracted with DCM (20 mL*2). The combined organic phase was dried over Na₂SO₄, filtered and the filtrate was concentrated to give the compound [(1R)-1-[[[2-[2-[tert-butyl(dimethyl)silyl]oxyethyl]-5-ethoxy-4-iodo-pyrazol-3-yl]methyl-methyl-amino]methyl]propyl]methanesulfonate (400 mg, 0.678 mmol, 86% yield) as a white solid. LCMS: m/z 590.3 (M+1).

Step 4. To a solution of [(1R)-1-[[[2-[2-[tert-butyl(dimethyl)silyl]oxyethyl]-5-ethoxy-4-iodo-pyrazol-3-yl]methyl-methyl-amino]methyl]propyl]methanesulfonate (350 mg, 0.593 mmol, 1 eq) 2-methyl-4-(1-tetrahydropyran-2-yl-3-vinyl-indazol-5-yl)pyrazol-3-ol (192 mg, 0.593 mmol, 1 eq) in DMF (10 mL) was added K₂CO₃ (246 mg, 1.78 mmol, 3 eq). The mixture was stirred at 80° C. for 1 hour. On completion the reaction mixture was quenched by addition H₂O (30 mL) and extracted with EA (30 mL*3). The combined organic layers were washed with NaCl (30 mL), filtered and concentrated under reduced pressure to give a residue. The residue was purified by column chromatography (SiO₂, Petroleum ether/Ethyl acetate=3:1) to give the compound (2R)—N-[[2-[2-[tert-butyl(dimethyl)silyl]oxyethyl]-5-ethoxy-4-iodo-pyrazol-3-yl]methyl]-N-methyl-2-[2-methyl-4-(1-tetrahydropyran-2-yl-3-vinyl-indazol-5-yl)pyrazol-3-yl]oxy-butan-1-amine (400 mg, 0.489 mmol, 82% yield) as a yellow solid. ¹H NMR (400 MHz, CDCl₃) δ=7.97 (br d, J=14.0 Hz, 1H), 7.63-7.48 (m, 3H), 7.03 (ddd, J=3.2, 11.6, 18 Hz, 1H), 6.10 (d, J=18.0 Hz, 1H), 5.69 (br d, J=9.6 Hz, 1H), 5.52 (d, J=11.6a Hz, 1H), 4.29-4.17 (m, 3H), 4.11-3.94 (m, 3H), 3.89-3.64 (m, 7H), 2.63-2.49 (m, 1H), 2.23-2.12 (m, 2H), 2.05 (br d, J=8.4 Hz, 1H), 1.96 (s, 2H), 1.78 (br s, 4H), 1.69-1.52 (m, 2H), 1.44-1.34 (m, 4H), 0.86-0.75 (m, 12H), -0.04-0.15 (m, 6H); LCMS: m/z 818.4 (M+1).

Step 5. A mixture of (2R)—N-[[2-[2-[tert-butyl(dimethyl)silyl]oxyethyl]-5-ethoxy-4-iodo-pyrazol-3-yl]methyl]-N-methyl-2-[2-methyl-4-(1-tetrahydropyran-2-yl-3-vinyl-indazol-5-yl)pyrazol-3-yl]oxy-butan-1-amine (400 mg, 0.489 mmol, 1 eq), Pd(OAc)₂ (21.9 mg, 0.098 mmol, 0.2 eq), TBAC (135 mg, 0.489 mmol, 136 L, 1 eq) and NaHCO₃ (1.22 mmol, 47.5 L, 2.5 eq) in DMF (5 mL) was degassed and purged with N₂ for 3 times, and then the mixture was stirred at 80° C. for 1 hour under N₂ atmosphere. On completion the reaction mixture was quenched by addition H₂O (15 mL) and extracted with EA (15 mL*3). The combined organic layers were washed with NaCl (15 mL), filtered and concentrated under reduced pressure to give the compound tert-butyl-[2-[(8R,17E)-15-ethoxy-8-ethyl-5,10-dimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaen-13-yl]ethoxy]-dimethyl-silane (210 mg, 0.304 mmol, 62% yield) as a white solid. LCMS: m/z 690.3 (M+1).

Step 6. To a solution of tert-butyl-[2-[(8R,17E)-15-ethoxy-8-ethyl-5,10-dimethyl-21-tetrahydropyran-2-yl-7-oxa-4,5,10,13,14,20,21-heptazapentacyclo[17.5.2.0^{2,6}0.0^{12,16}. 0^{22,26}]hexacosa-1(25),2(6),3,12(16),14,17,19,22(26),23-nonaen-13-yl]ethoxy]-dimethyl-silane (280 mg, 0.405 mmol, 1 eq) in DCM (5 mL) was added TFA (46.2 mg, 0.405 mmol, 30.1 μL, 1 eq).The mixture was stirred at 25° C. for 1 hour. On completion the reaction mixture was filtered and the filtrate was concentrated under reduced pressure to remove solvent. The crude product was purified by reversed-phase column: Phenomenex luna C18 150*25 mm*10 um; mobile phase: [water(FA)-ACN];gradient:14%-44% B over 11 min to give 2-[(11S,17E)-16-ethoxy-11-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (1.77 mg, 0.0035 mmol, 8.52e-1% yield, 96% purity) as a white solid and 2-[(10S,17E)-16-ethoxy-10-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (4.13 mg, 0.0082 mmol, 2.01% yield, 97% purity) as a white solid.

### Preparation of (2S)—N-methyl-2-[2-methyl-4-(1-tetrahydropyran-2-yl-3-vinyl-indazol-5-yl)pyrazol-3-yl]oxy-propan-1-amine (I-261)

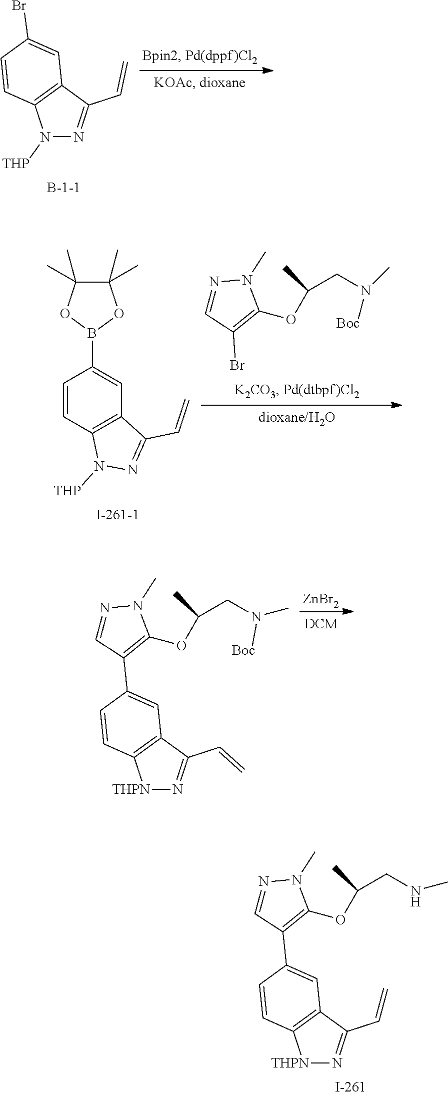

Step 1. A mixture of 5-bromo-1-tetrahydropyran-2-yl-3-vinyl-indazole (8.00 g, 26.0 mmol, 1 eq), 4,4,5,5-tetramethyl-2-(4,4,5,5-tetramethyl-1,3,2-dioxaborolan-2-yl)-1,3,2-dioxaborolane (7.27 g, 28.6 mmol, 1.1 eq), potassium; acetate (3.83 g, 39.1 mmol, 1.5 eq), cyclopentyl(diphenyl)phosphane; dichloropalladium; iron (1.91 g, 2.60 mmol, 0.1 eq) in dioxane (150 mL) was degassed and purged with N₂ for 3 times. Then the mixture was stirred at 90° C. for 5 h under N₂ atmosphere. On completion, the mixture was concentrated to give 1-tetrahydropyran-2-yl-5-(4,4,5,5-tetramethyl-1,3,2-dioxaborolan-2-yl)-3-vinyl-indazole (9.20 g, crude) as yellow oil. LCMS: m/z 355.1 (M+1)

Step 2. A mixture of tert-butyl N-[(2R)-2-(4-bromo-2-methyl-pyrazol-3-yl)oxypropyl]-N-methyl-carbamate (8.45 g, 24.3 mmol, 1 eq) from Ex. 89, 1-tetrahydropyran-2-yl-5-(4,4,5,5-tetramethyl-1,3,2-dioxaborolan-2-yl)-3-vinyl-indazole (8.60 g, 24.3 mmol, 1 eq), K₂CO₃ (6.71 g, 48.6 mmol, 2 eq), ditert-butyl(cyclopentyl)phosphane;dichloropalladium;iron (1.58 g, 2.43 mmol, 0.1 eq) in dioxane (140 mL) and H₂O (28 mL) was degassed and purged with N₂ for 3 times, and then the mixture was stirred at 80° C. for 2 h under N₂ atmosphere. On completion, the mixture was dried over Na₂SO₄, concentrated to give a residue. The residue was purified by column chromatography (SiO₂, Petroleum ether/Ethyl acetate=1:0 to 1:1) to give tert-butyl N-methyl-N-[(2S)-2-[2-methyl-4-(1-tetrahydropyran-2-yl-3-vinyl-indazol-5-yl) pyrazol-3-yl]oxypropyl]carbamate (4.40 g, 8.88 mmol, 36% yield) as brown oil. LCMS: m/z 496.2 (M+1).

Step 3. To a solution of tert-butyl N-methyl-N-[(2S)-2-[2-methyl-4-(1-tetrahydropyran-2-yl-3-vinyl-indazol-5-yl)pyrazol-3-yl]oxypropyl]carbamate (500 mg, 1.01 mmol, 1 eq) in DCM (5 mL) was added ZnBr₂ (681 mg, 3.03 mmol, 3 eq). The mixture was stirred at 25° C. for 16 h. On completion, the reaction was concentrated under vacuum to give (2S)—N-methyl-2-[2-methyl-4-(1-tetrahydropyran-2-yl-3-vinyl-indazol-5-yl)pyrazol-3-yl]oxy-propan-1-amine (2.00 g, 0.961 mmol, 95% yield, 19% purity) as a yellow oil. LCMS: m/z 396.2 (M+1).

### General Method W: Preparation of 2-[(10S,17E)-16-(methoxymethyl)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 261)

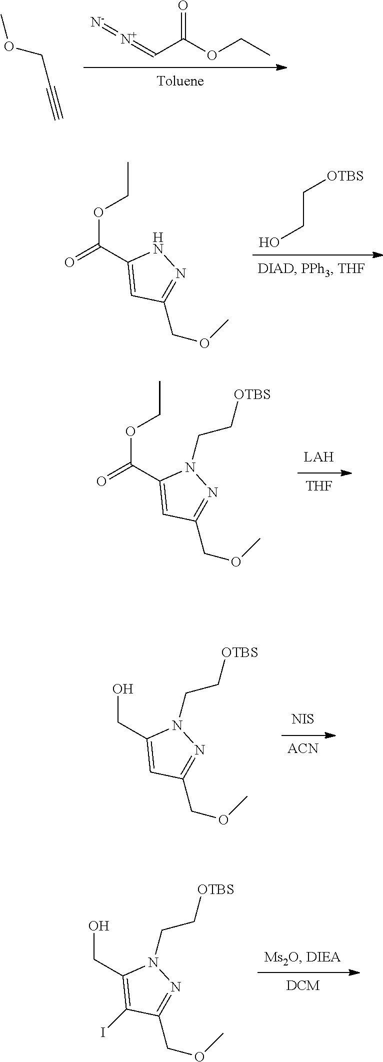

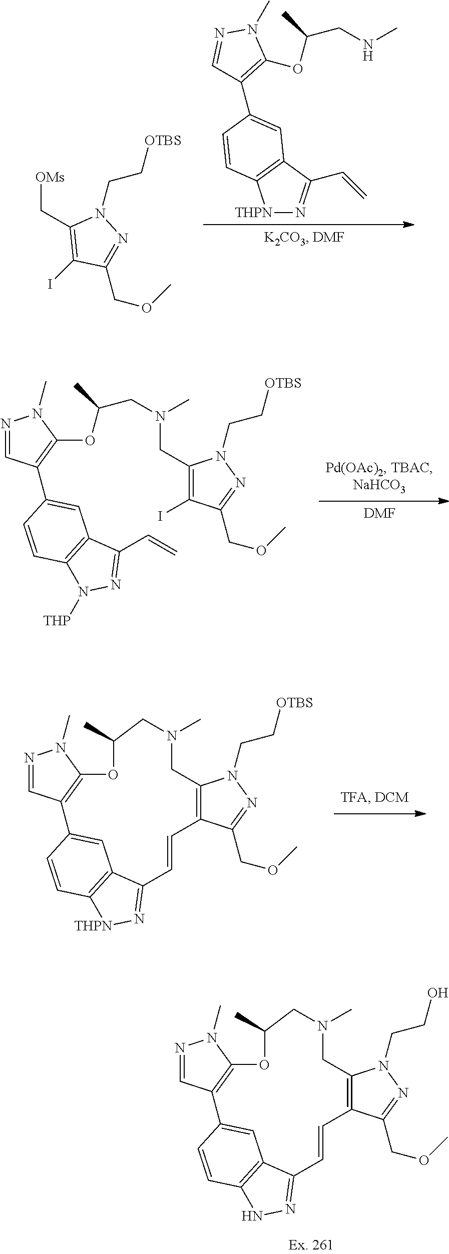

Step 1. A mixture of 3-methoxyprop-1-yne (15.0 g, 214 mmol, 17.6 mL, 1 eq) and ethyl 2-diazoacetate (24.4 g, 214 mmol, 1 eq) in toluene (150 mL) was stirred at 115° C. for 2 h. On completion, the reaction mixture was concentrated under reduced pressure to give a residue. The residue was purified by flash silica gel chromatography (ISCO®; 220 g SepaFlash® Silica Flash Column, Eluent of 0-1% THF/Petroleum ether gradient @100 m/min) to give ethyl 3-(methoxymethyl)-1H-pyrazole-5-carboxylate (6.15 g, 28.7 mmol, 13% yield, 86% purity) as a yellow oil. ¹H NMR (400 MHz, CDCl₃-d) δ=6.82 (s, 1H), 4.54 (s, 2H), 4.40 (q, J=7.2 Hz, 2H), 3.42 (s, 3H), 1.41 (t, J=7.2 Hz, 3H); LCMS: m/z 185.1 (M+1).

Step 2. To a mixture of ethyl 3-(methoxymethyl)-1H-pyrazole-5-carboxylate (5.95 g, 32.3 mmol, 1 eq), 2-[tert-butyl(dimethyl)silyl]oxyethanol (11.4 g, 64.6 mmol, 2 eq) and PPh₃ (18.6 g, 71.1 mmol, 2.2 eq) in THE (60 mL) was added DIAD (14.4 g, 71.1 mmol, 13.8 mL, 2.2 eq) at 0° C. The mixture was stirred at 25° C. for 2 h. On completion, the reaction mixture was concentrated under reduced pressure to give a residue. The residue was purified by flash silica gel chromatography (ISCO®; X g SepaFlash® Silica Flash Column, Eluent of 0-5% THF/Petroleum ether gradient @100 mL/min) to give ethyl 2-[2-[tert-butyl (dimethyl) silyl]oxyethyl]-5-(methoxymethyl)pyrazole-3-carboxylate (13.0 g, 32.3 mmol, 99% yield, 85% purity) as a yellow oil. ¹H NMR (400 MHz, CDCl₃-d) δ=6.84 (s, 1H), 4.70 (t, J=5.6 Hz, 2H), 4.47 (s, 2H), 4.33 (q, J=7.2 Hz, 2H), 3.97-3.91 (m, 2H), 3.42-3.37 (m, 3H), 1.37 (t, J=7.2 Hz, 3H), 0.83-0.79 (m, 10H), -0.05-0.11 (m, 7H); LCMS: m/z 343.5 (M+1).

Step 3. To a mixture of ethyl 2-[2-[tert-butyl (dimethyl)silyl]oxyethyl]-5-(methoxy methyl) pyrazole-3-carboxylate (12.5 g, 31.0 mmol, 1 eq) in THE (130 mL) was added LiAlH₄ (2.5 M, 13.7 mL, 1.1 eq) at 0° C. The mixture was stirred at 0° C. for 2 h. On completion, the reaction mixture was quenched by addition of 15% NaOH. The combined organic layers were dried over Na₂SO₄, filtered and concentrated under reduced pressure to give a residue. The residue was purified by flash silica gel chromatography (TSCO®; 80 g SepaFlash® Silica Flash Column, Eluent of 0-50% Ethyl acetate/Petroleum ether gradient @100 mL/min) to give [2-[2-[tert-butyl(dimethyl)silyl]oxyethyl]-5-(methoxymethyl)pyrazol-3-yl]methanol (4.00 g, 12.9 mmol, 42% yield, 97% purity) as a yellow oil. ¹H NMR (400 MHz, CDCl3-d) δ=6.23 (s, 1H), 4.62 (s, 2H), 4.45 (s, 2H), 4.30 (t, J=4.8 Hz, 2H), 4.01 (t, J=4.8 Hz, 2H), 3.39 (s, 3H), 0.82 (s, 9H), 0.00-0.02 (m, 6H); LCMS: m/z 301.4 (M+1).

Step 4. To a mixture of [2-[2-[tert-butyl(dimethyl)silyl]oxyethyl]-5-(methoxymethyl)pyrazol-3-yl]methanol (1.00 g, 3.33 mmol, 1 eq) in ACN (10 mL) was added NIS (599 mg, 2.66 mmol, 0.8 eq) at 0° C. The mixture was stirred at 25° C. for 1 h. Then NIS (449 mg, 1.77 mmol, 0.6 eq) was added and the reaction mixture was stirred at 25° C. for 1 h. On completion, the mixture was quenched with sat. Na₂SO₃ (50 mL) at 0° C. and extracted with ethyl acetate (50 mL×3), the combined organic phase was dried over anhydrous Na₂SO₄, filtered and the filtrate was concentrated to give a residue. The residue was purified by column chromatography (SiO₂, Petroleum ether/Ethyl acetate=0/1 to 1/1) to give [2-[2-[tert-butyl (dimethyl)silyl]oxyethyl]-4-iodo-5-(methoxymethyl)pyrazol-3-yl]methanol (637 mg, 1.37 mmol, 41% yield, 92% purity) as a light yellow oil. LCMS: m/z 427.3 (M+1).

Step 5. To a mixture of [2-[2-[tert-butyl(dimethyl)silyl]oxyethyl]-4-iodo-5-(methoxymethyl)pyrazol-3-yl]methanol (587 mg, 1.38 mmol, 1 eq) and DIEA (534 mg, 4.13 mmol, 719 μL, 3 eq) in DCM (5 mL) was added methylsulfonyl methanesulfonate (479 mg, 2.75 mmol, 2 eq) at 0° C. The mixture was stirred at 25° C. for 2 h. On completion, the mixture was poured into water (50 mL) aqueous solution, the aqueous phase was extracted with DCM (50 mL×2). The combined organic phase was washed with brine (30 mL×2), dried over anhydrous Na₂SO₄, filtered and concentrated under vacuum to give [2-[2-[tert-butyl(dimethyl) silyl]oxyethyl]-4-iodo-5-(methoxymethyl)pyrazol-3-yl]methyl methanesulfonate (694 mg, crude) was obtained as a yellow oil. LCMS: m/z 505.0 (M+1).

Step 6. To a mixture of [2-[2-[tert-butyl(dimethyl)silyl]oxyethyl]-4-iodo-5-(methoxymethyl)pyrazol-3-yl]methyl methanesulfonate (686 mg, 1.36 mmol, 0.25 eq) and (2S)—N-methyl-2-[2-methyl-4-(1-tetrahydropyran-2-yl-3-vinyl-indazol-5-yl) pyrazol-3-yl]oxy-propan-1-amine (2.15 g, 5.44 mmol, 1 eq) in DMF (20 mL) was added K₂CO₃ (2.25 g, 16.3 mmol, 3 eq). The mixture was stirred at 80° C. for 1 h. On completion, the mixture was poured into water (80 mL) aqueous solution, the aqueous phase was extracted with ethyl acetate (60 mL×2). The combined organic phase was washed with brine (50 mL×2), dried over anhydrous Na₂SO₄, filtered and concentrated under vacuum. The residue was purified by column chromatography (SiO₂, Petroleum ether/Ethyl acetate=0/1 to 1/1) to give (2S)—N-[[2-[2-[tert-butyl (dimethyl) silyl]oxyethyl]-4-iodo-5-(methoxymethyl)pyrazol-3-yl]methyl]-N-methyl-2-[2-methyl-4-(1-tetrahydropyran-2-yl-3-vinyl-indazol-5-yl)pyrazol-3-yl]oxy-propan-1-amine (393 mg, 0.489 mmol, 9% yield) as a yellow oil. ¹H NMR (400 MHz, CDCl₃-d) δ=8.00 (s, 1H), 7.58 (s, 1H), 7.55 (s, 2H), 6.99 (s, 1H), 6.12 (d, J=18.0 Hz, 1H), 5.69 (br d, J=9.6 Hz, 1H), 5.54 (d, J=11.4 Hz, 1H), 4.40-4.33 (m, 1H), 4.32-4.23 (m, 1H), 4.22-4.14 (m, 1H), 4.08 (d, J=11.0 Hz, 1H), 3.90 (t, J=5.2 Hz, 2H), 3.81-3.73 (m, 2H), 3.71 (s, 3H), 3.61 (br d, J=4.0 Hz, 2H), 3.39 (s, 3H), 3.00-2.86 (m, 2H), 2.68-2.51 (m, 2H), 2.43 (dd, J=6.0, 12.8 Hz, 1H), 2.21-2.13 (m, 1H), 2.06 (s, 3H), 1.78 (t, J=9.2 Hz, 2H), 1.70-1.62 (m, 1H), 1.57 (s, 3H), 0.78 (s, 9H), -0.12 (d, J=4.0 Hz, 6H). LCMS: m/z 804.3 (M+1).

Steps 7 and 8 were performed in a similar manner to General Method V to give 2-[(10S,17E)-16-(methoxymethyl)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (7.81 mg, 0.016 mmol, 39% yield, 100% purity) as a white solid.

### Preparation of 1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}-2-methylpropan-2-ol (Ex. 262) and 1-{(10S,17E)-12-ethyl-8,10-dimethyl-16-[(propan-2-yl)oxy]-2,8,10,11.12,13-hexahydro-14H-3,5-ethenotripyrazolo[3,4-f:3′,4′-j:4″,3″-n][1,4]oxazacyclopentadecin-14-yl}propan-2-one (Ex. 263)

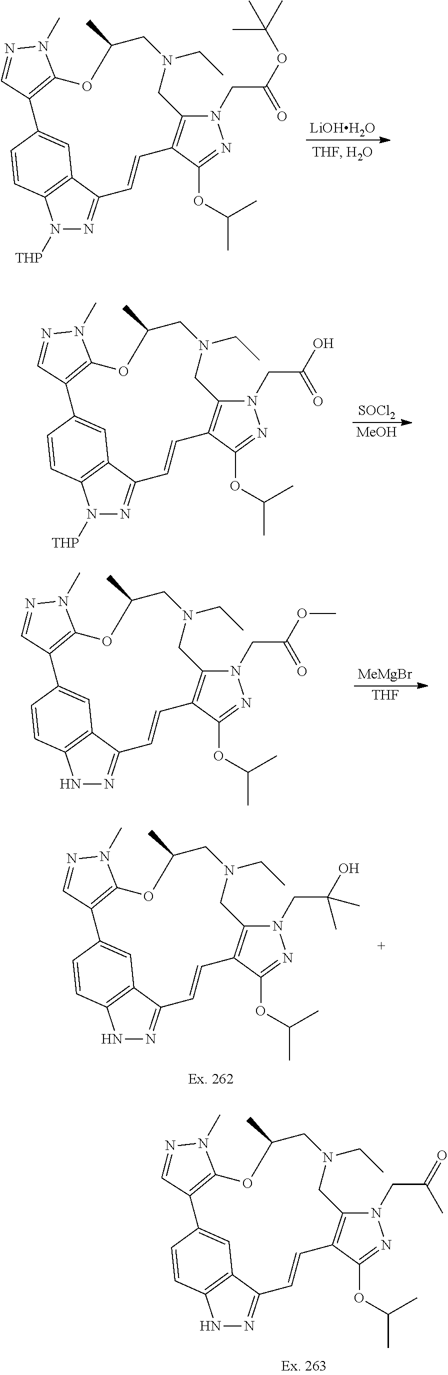

Step 1. A mixture of tert-butyl 2-[(8S,17E)-10-ethyl-15-isopropoxy-5,8-dimethyl-21-tetrahydropyran-2-yl-7-oxa-4,5,10,13,14,20,21-heptazapentacyclo [17.5.2.02,6.012, 16.022,26]hexacosa-1(25),2(6),3,12(16),14,17,19,22(26),23-nonaen-13-yl]acetate (60.0 mg, 0.091 mmol, 1 eq, from Ex. 232) in H₂O (0.25 mL) and THF (0.5 mL) was added LiOH·H₂O (6.53 mg, 0.272 mmol, 3 eq) at 0° C. Then the mixture was stirred at 25° C. for 1 h. On completion, the reaction mixture was concentrated in vacuo to give 2-[(8S,17E)-10-ethyl-15-isopropoxy-5, 8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaen-13-yl]acetic acid (55.0 mg, 0.072 mmol, 79% yield, 79% purity) as a brown solid. LCMS: m/z 604.1 (M+1)

Step 2. To a solution of 2-[(8S,17E)-10-ethyl-15-isopropoxy-5,8-dimethyl-21-tetrahydropyran-2-yl-7-oxa-4,5,10,13,14,20,21-heptazapentacyclo [17.5.2.0^{2,6}.0^{12,16}.0^{22,26}]hexacosa-1(25),2(6),3,12(16),14,17,19,22(26),23-nonaen-13-yl]acetic acid (54.0 mg, 0.089 mmol, 1 eq) in MeOH (1 mL) was added SOCl₂ (19.1 mg, 0.161 mmol, 1.8 eq) at −15° C. The mixture was stirred at 25° C. for 16 h. On completion, the solvent was lyophilized and to give desired compound to give methyl 2-[(8S,17E)-10-ethyl-15-isopropoxy-5,8-dimethyl-7-oxa-4,5,10,13,14,20,21-heptazapentacyclo[17.5.2.02, 6.012,16.022,26]hexacosa-1(25),2(6),3,12(16),14,17,19,22(26),23-nonaen-13-yl]acetate (45.0 mg, 0.069 mmol, 77% yield, 82% purity) as a yellow solid. LCMS: m/z 534.1 (M+1)

Step 3. A mixture of methyl 2-[(8S,17E)-10-ethyl-15-isopropoxy-5,8-dimethyl-7-oxa-4,5,10,13,14,20,21-heptazapentacyclo[17.5.2.02,6.012,16.022,26]hexacosa-1(25),2(6),3,12(16),14,17,19,22(26),23-nonaen-13-yl]acetate (40.0 mg, 0.075 mmol, 1 eq) in THF (1 mL) was degassed and purged with N₂ for 3 times, and then MeMgBr (3 M, 125 μL, 5 eq) was added at 0° C. Then the mixture was stirred at 25° C. for 4 h under N₂ atmosphere. On completion, the mixture was quenched with MeOH (1 mL). The mixture was purified by Prep-HPLC (column: Phenomenex luna C18 150*25 mm*10 um; mobile phase: [water(FA)-ACN];gradient:24%-54% B over 8 min) and lyophilized to give Ex. 262 (1.94 mg, 0.003 mmol, 4% yield, 93.29% purity, FA) as a white solid, and Ex. 263 (1.54 mg, 0.003 mmol, 4% yield, 100% purity, FA) as a white solid.

### Preparation of (10R,17E)-14-(2-hydroxyethyl)-8,12-dimethyl-16-[(propan-2-yl)oxy]-2,10,11,12,13,14-hexahydro-8H-3,5-ethenotripyrazolo[3,4-f:3′,4′-j:4″,3″-n][1,4]oxazacyclopentadecine-10-carbonitrile (Ex. 264)

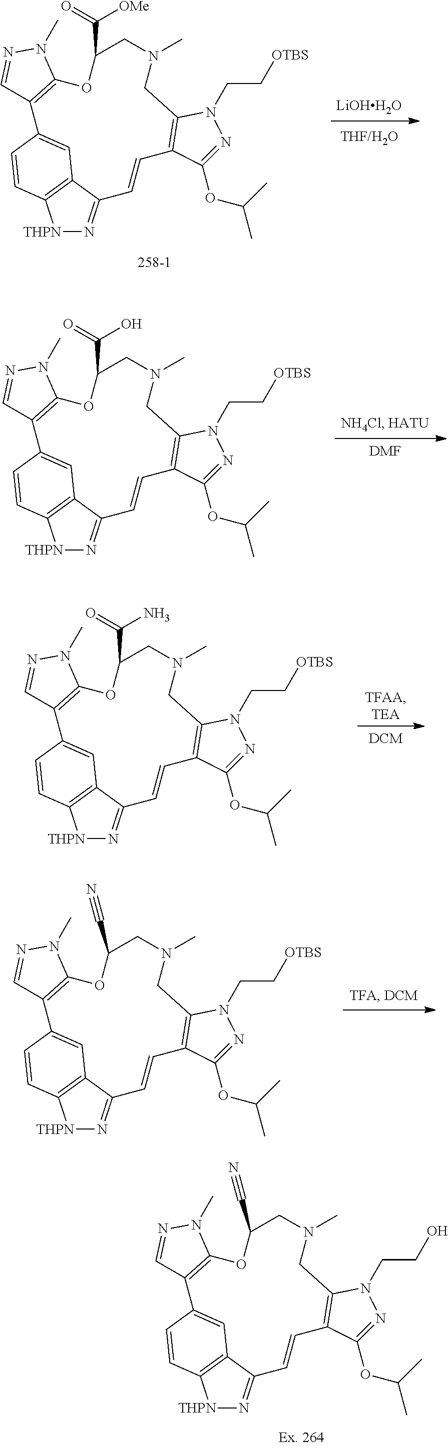

Step 1. To a solution of methyl (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene-8-carboxylate (700 mg, 0.953 mmol, 1 eq) in THE (9 mL) and H₂O (3 mL) was added LiOH·H₂O (120 mg, 2.86 mmol, 3 eq). The mixture was stirred at 25° C. for 2 h. On completion. The mixture was quenched with water (20 mL) and extracted with ethyl acetate (25 mL×3), the combined organic phase was dried over anhydrous sodium sulfate, filtered and concentrated to give (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene-8-carboxylic acid (700 mg, crude) as a white solid. LCMS: m/z 720.3 (M+1).

Step 2. To a solution of (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo [17.5.2.0^{2,6}.0^{12,16}0.0^{22,26}]hexacosa-1(25),2(6),3,12(16),14,17,19,22(26),23-nonaene-8-carboxylic acid (700 mg, 0.972 mmol, 1 eq) in DMF (20 mL) was added NH₄Cl (520 mg, 9.72 mmol, 10 eq), DIEA (376 mg, 2.92 mmol, 3 eq), and then HATU (1.11 g, 2.92 mmol, 3 eq) was added dropwise at 0° C. The mixture was stirred at 25° C. for 2 h. On completion, the mixture was quenched with water (20 mL) and extracted with ethyl acetate (25 mL×3), the combined organic phase was dried over anhydrous sodium sulfate, filtered and concentrated to give a residue. The residue was purified by column chromatography (SiO₂, PE/EA=2:1 to 1:1) to give (8R,17E)-13-[2-[tert-butyl(dimethyl) silyl]oxyethyl]-15-isopropoxy-5,10-dimethyl-21-tetrahydropyran-2-yl-7-oxa-4,5,10, 13,14,20,21-heptazapentacyclo[17.5.2.02,6.012,16.022,26]hexacosa-1(25),2(6),3, 12(16),14,17,19,22(26),23-nonaene-8-carboxamide (500 mg, 0.695 mmol, 71% yield) as a white solid. LCMS: m/z 719.2 (M+1).

Step 3. To a solution of (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo [17.5.2.0^{2,6}.0^{12,16}.0^{22,26}]hexacosa-1(25),2(6),3,12(16),14,17,19,22(26),23-nonaene-8-carboxamide (480 mg, 0.667 mmol, 1 eq) in CHCl₃ (10 mL) was added TEA (229 mg, 2.27 mmol, 3.4 eq) and TFAA (238 mg, 1.13 mmol, 1.7 eq). The mixture was stirred at 0° C. for 2 h. On completion, The mixture was quenched with water (20 mL) and extracted with DCM (25 mL×3), the combined organic phase was dried over anhydrous sodium sulfate, filtered and concentrated to give (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene-8-carbonitrile (400 mg, 0.570 mmol, 85% yield) as a yellow oil. LCMS: m/z 701.7 (M+1).

Step 4. To a solution of (8R,17E)-13-[2-[tert-butyl(dimethyl)silyl]oxyethyl]-15-isopropoxy-5,10-dimethyl-21-tetrahydropyran-2-yl-7-oxa-4,5,10,13,14,20,21-heptazapentacyclo [17.5.2.0^{2,6}.0^{12,16}0.0^{22,26}]hexacosa-1(25),2(6),3,12(16),14,17,19,22(26),23-nonaene-8-carbonitrile (400 mg, 0.570 mmol, 1 eq) in DCM (3 mL) was added TFA (33.6 mmol, 2.50 mL, 59 eq). The mixture was stirred at 25° C. for 2 h. On completion, the mixture was filtered and concentrated to give a residue. The crude product was triturated with EA (10 ml) at 25° C. for 5 mines to give (8R,17E)-13-(2-hydroxyethyl)-15-isopropoxy-5,10-dimethyl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaene-8-carbonitrile (132.93 mg, 0.264.50 mmol, 46% yield) as a white solid.

### Preparation of 2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 265)

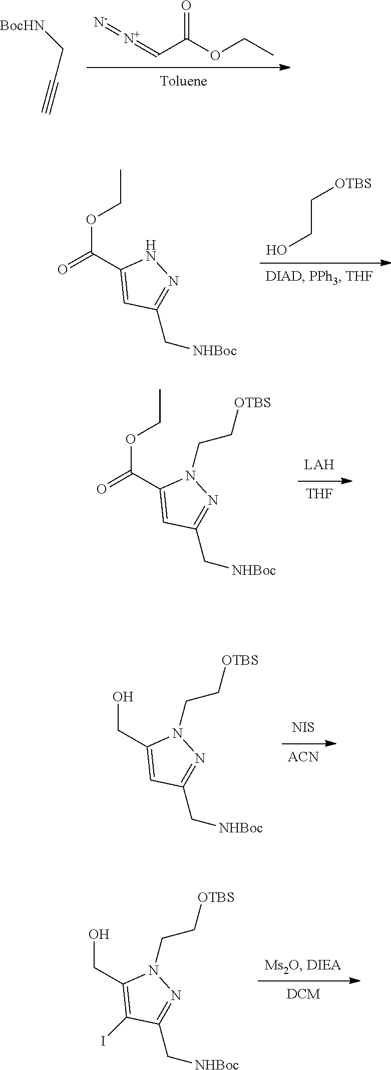

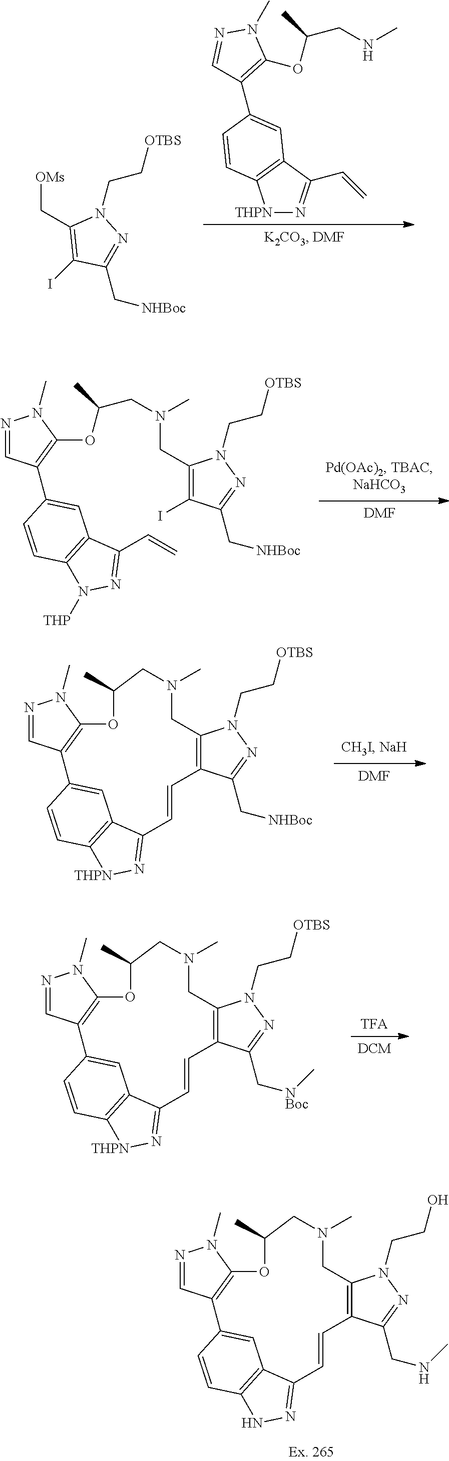

Steps 1 through 7 followed the procedures of General Method W steps 1 through 7.

Step 8. To a solution of tert-butyl N-[[(8S,17E)-13-[2-[tert-butyl(dimethyl)silyl]oxyethyl]-5,8,10-trimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaen-15-yl]methyl]carbamate (0.250 g, 0.328 mmol, 1.00 eq) in DMF (2.50 mL) was added NaH (26.3 mg, 0.657 mmol, 60% purity, 2.00 eq) at 0° C. The mixture was stirred at 0° C. for 0.5 h. CH₃I (69.9 mg, 0.492 mmol, 30.7 μL, 1.50 eq) was added into the mixture at 0° C. The mixture was stirred at 25° C. for 3.5 h. On completion, the mixture was poured into NH₄Cl (50.0 mL) aqueous, the aqueous phase was extracted with ethyl acetate (20.0 mL×3). The combined organic phase was washed with brine (20.0 mL×2), dried over anhydrous Na₂SO₄, filtered and concentrated under vacuum to give tert-butyl N-[[(8S,17E)-13-[2-[tert-butyl(dimethyl)silyl]oxyethyl]-5,8,10-trimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaen-15-yl]methyl]-N-methyl-carbamate (0.170 g, 0.219 mmol, 67% yield) as a yellow solid. LCMS: m/z 775.6 (M+1).

Step 9 was performed in a similar manner to the last step in General Method W to give 2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (2.6 mg, 0.0053 mmol, 3% yield, 96.4% purity, FA) as a white solid.

### Preparation of 2-{(10S,17E)-16-[(dimethylamino)methyl]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 266)

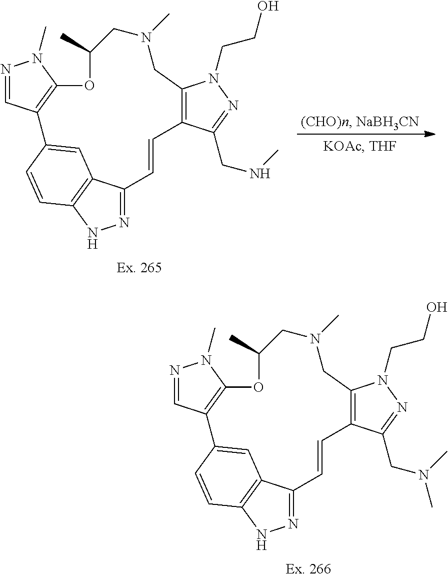

Step 1. To a solution of 2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (40.0 mg, 0.084 mmol, 1.00 eq) in THE (0.100 mL) was added KOAc (82.4 mg, 0.839 mmol, 10.0 eq) and HCHO (12.6 mg, 0.420 mmol, 11.6 μL, 5.00 eq). The mixture was stirred at 25° C. for 0.5 h. NaBH₃CN (10.6 mg, 0.168 mmol, 2.00 eq) was added into the mixture. On completion, the mixture was stirred at 25° C. for 0.5 h. The reaction was concentrated under vacuum. The residue was purified by prep-HPLC (column: Welch Ultimate C18 150*25 mm*5 um; mobile phase: [water(FA)-ACN];gradient:0%-30% B over 10 min) to give 2-{(10S,17E)-16-[(dimethylamino)methyl]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol (0.52 mg, 9.38e-4 mmol, 1% yield, 96.8% purity, FA) as a yellow solid.

### Preparation of (10S,18E)-16-ethoxy-8,10,12-trimethyl-2,8,10,11,12,13-hexahydro-3,5-ethenodipyrazolo[3,4-j:4′,3′-n]pyrido[3,2-f][1,4]oxazacyclopentadecine (Ex. 267)

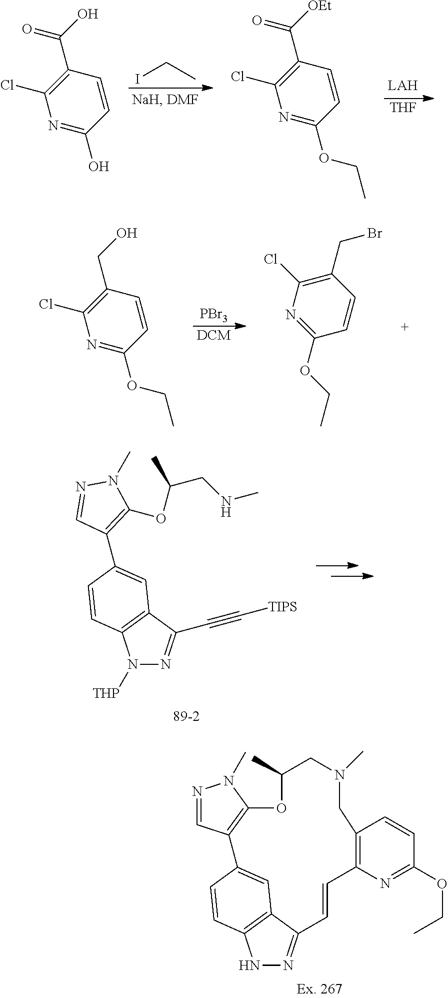

Step 1. To a solution of 2-chloro-6-hydroxy-pyridine-3-carboxylic acid (6.00 g, 34.5 mmol, 1 eq) in DMF (120 mL) at 0° C., then was added NaH (4.98 g, 124 mmol, 60% purity, 3.6 eq) at 0° C. for 0.5 h. EtI (26.9 g, 172 mmol, 13.8 mL, 5 eq) was added at 0° C. The mixture was stirred at 25° C. for 16 hours. On completion, the mixture was slowly quenched with water (300 mL), extracted with EA (3*300 mL). The combined organic layers were washed with brine (3*300 mL), dried over Na₂SO₄, filtered and concentrated in vacuum. The residue was purified by column chromatography (SiO₂, PE/THF=1/0 to 20/1) to give ethyl 2-chloro-6-ethoxy-pyridine-3-carboxylate (4.40 g, 19.1 mmol, 55% yield) as a yellow oil. 1H NMR (400 MHz, CHLOROFORM-d) δ=8.12 (d, J=8.4 Hz, 1H), 6.68 (d, J=8.4 Hz, 1H), 4.52-4.25 (m, 4H), 1.40 (dt, J=2.8, 7.1 Hz, 6H). LCMS: m/z 230.0 (M+1)

Step 2. To a solution of ethyl 2-chloro-6-ethoxy-pyridine-3-carboxylate (4.40 g, 19.1 mmol, 1 eq) in THF (44 mL) was added LiAlH₄ (2.5 M, 9.20 mL, 1.2 eq) at 0° C. The mixture was stirred at 25° C. for 16 h. On completion, the mixture was slowly added to water (0.9 ml), 15% sodium hydroxide (0.9 ml) and was added water (2.7 ml) to quench. The reaction mixture was filtered and concentrated under reduced pressure to give a residue. The residue was purified by silica gel column chromatography (20 g silica gel column, THF in PE from 0% to 20%) to give (2-chloro-6-ethoxy-3-pyridyl) methanol (2.85 g, 15.1 mmol, 79% yield) as a yellow oil. ¹H NMR (400 MHz, CDCl₃) δ 7.68 (d, J=8.4 Hz, 1H), 6.67 (d, J=8.4 Hz, 1H), 4.69 (s, 2H), 4.35 (q, J=7.2 Hz, 2H), 2.09 (d, J=11.2 Hz, 1H), 1.39 (t, J=7.2 Hz, 3H). LCMS: m/z 188.0 (M+1)

Step 3. To a solution of (2-chloro-6-ethoxy-3-pyridyl)methanol (2.85 g, 15.1 mmol, 1 eq) in DCM (30 mL) at 0° C. was added PBr₃ (4.93 g, 18.2 mmol, 1.2 eq). The mixture was stirred at 25° C. for 16 h. On completion, the saturated sodium bicarbonate was quenched and the pH of the reaction solution was adjusted to 8 and extracted with DCM (3*100 mL). The combined organic layers were washed with brine (3*100 mL), dried over Na₂SO₄ and concentrated to give 3-(bromomethyl)-2-chloro-6-ethoxy-pyridine (3.60 g, 14.3 mmol, 94% yield) as a yellow solid. ¹H NMR (400 MHz, CDCl₃) δ 7.62 (d, J=8.4 Hz, 1H), 6.66 (d, J=8.4 Hz, 1H), 4.54 (s, 2H), 4.37 (q, J=7.2 Hz, 2H), 1.39 (t, J=7.2 Hz, 3H). LCMS: m/z 249.9 (M+1)

The remaining steps were performed according to Ex. 151 from 3-(bromomethyl)-2-chloro-6-ethoxy-pyridine and 89-2 to give Ex. 267.

### General Method X: Preparation of (2R)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile (Ex. 268) and (2S)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile (Ex. 269)

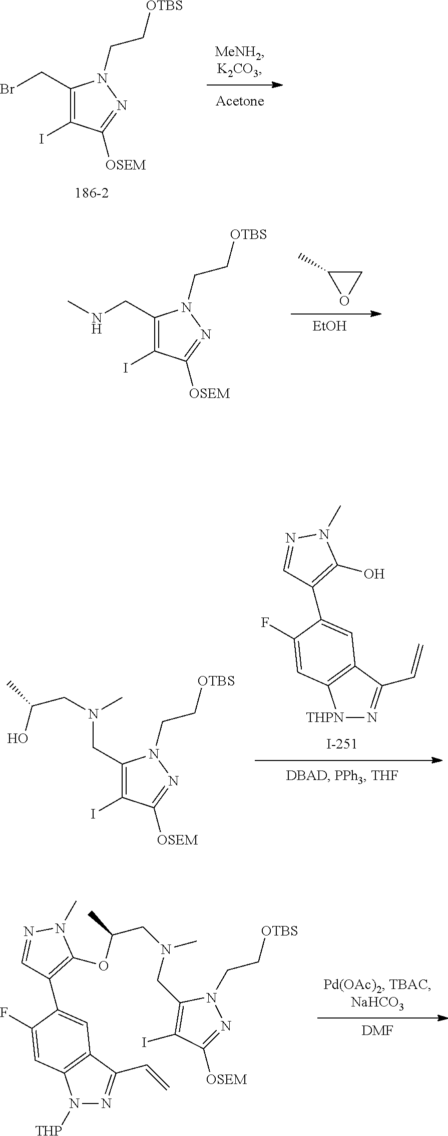

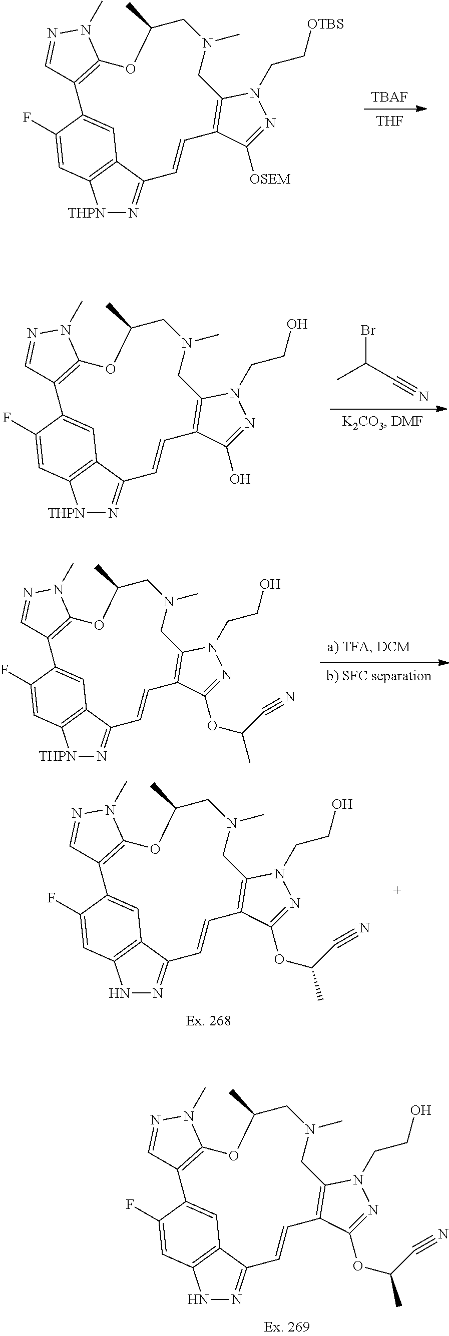

Steps 1 and 2 were performed in a similar manner to the synthesis of I-246.

Steps 3 and 4 were performed in a similar manner to steps 1 and 2 in General Method U.

Step 5. To a solution of tert-butyl-[2-[(8S,17E)-24-fluoro-5,8,10-trimethyl-21-tetrahydropyran-2-yl-15-(2-trimethylsilylethoxymethoxy)-7-oxa-4,5,10,13,14,20,21-heptazapentacyclo[17.5.2.0^{2,6}.0^{12,16}.0^{22,26}]hexacosa-1(25),2(6),3,12(16),14,17,19,22(26), 23-nonaen-13-yl]ethoxy]-dimethyl-silane (100 mg, 0.126 mmol, 1 eq) in THE (1 mL) was added TBAF (1 M, 754 μL, 6 eq), then the mixture was stirred at 70° C. for 3 h. On completion, the reaction mixture was diluted with H₂O (5 mL) and extracted with EA (2 mL*3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0-10% MeOH/DCM ether gradient@20 mL/min) to give (8S,17E)-24-fluoro-13-(2-hydroxyethyl)-5,8,10-trimethyl-21-tetrahydropyran-2-yl-7-oxa-4,5,10,13,14,20,21-heptazapentacyclo[17.5.2.02,6.012,16.022,26]hexacosa-1(25),2(6),3,12(16),14,17,19, 22(26),23-nonaen-15-ol (40.0 mg, 0.073 mmol, 58% yield) as a yellow oil. LCMS: m/z 552.2 (M+1).

Step 6. To a solution of (8S,17E)-24-fluoro-13-(2-hydroxyethyl)-5,8,10-trimethyl-21-tetrahydropyran-2-yl-7-oxa-4,5,10,13,14,20,21-heptazapentacyclo [17.5.2.02,6.012, 16.022,26]hexacosa-1(25),2(6),3,12(16),14,17,19,22(26),23-nonaen-15-ol (30.0 mg, 0.054 mmol, 1 eq) in DMF (1 mL) was added 2-bromopropanenitrile (10.9 mg, 0.082 mmol, 1.5 eq) and K₂CO₃ (22.6 mg, 0.163 mmol, 3 eq), then the mixture was stirred at 60° C. for 2 h. On completion, the reaction mixture was diluted with H₂O (3 mL) and extracted with EA (2 mL*3). The combined organic layers were washed with Brine (3 mL * 2), dried over Na₂SO₄, filtered and concentrated under reduced pressure to give 2-[[(8S,17E)-24-fluoro-13-(2-hydroxyethyl)-5,8,10-trimethyl-21-tetrahydropyran-2-yl-7-oxa-4,5,10,13, 14,20,21-heptazapentacyclo [17.5.2.02,6.012,16.022,26]hexacosa-1(25),2(6),3,12(16), 14,17,19,22(26),23-nonaen-15-yl]oxy]propanenitrile (40.0 mg, crude) as a yellow oil. LCMS: m/z 605.2 (M+1).

Step 7. To a solution of 2-[[(8S,17E)-24-fluoro-13-(2-hydroxyethyl)-5,8,10-trimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaen-15-yl]oxy]propane nitrile (30.0 mg, 0.050 mmol, 1 eq) in DCM (0.5 mL) was added TFA (461 mg, 4.04 mmol, 81.4 eq), then the mixture was stirred at 25° C. for 1 h. The reaction mixture was concentrated under reduced pressure to give a residue. The residue was purified by prep-HPLC (column: Waters xbridge 150*25 mm 10 um; mobile phase: [water(NH₄HCO₃)-ACN];gradient:28%-58% B over 10 min) to give desired compound (30 mg) as a white solid, which was further separated by SFC (column: DAICEL CHIRALCEL OD (250 mm*30 mm, 10 um);mobile phase: [C02-MeOH (0.1% NH₃H₂O)]; B %:25%, isocratic elution mode) to give (2R)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile (1.31 mg, 0.003 mmol, 5% yield) and (2S)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile (0.8 mg, 0.002 mmol, 3% yield) as a white solid.

### Preparation of 2-[(4aS,7aS,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol (Ex. 270) and 2-[(4aR,7aR,13E)-12-ethoxy-3,6,8-trimethyl-4a,5,6,7,7a,8,9,16-octahydro-17,19-ethenotripyrazolo[³,4-f:3′, j:4″,3″-n]pyrrolo[3,4-b][1,4]oxazacyclopentadecin-10(3H)-yl]ethan-1-ol (Ex. 271)

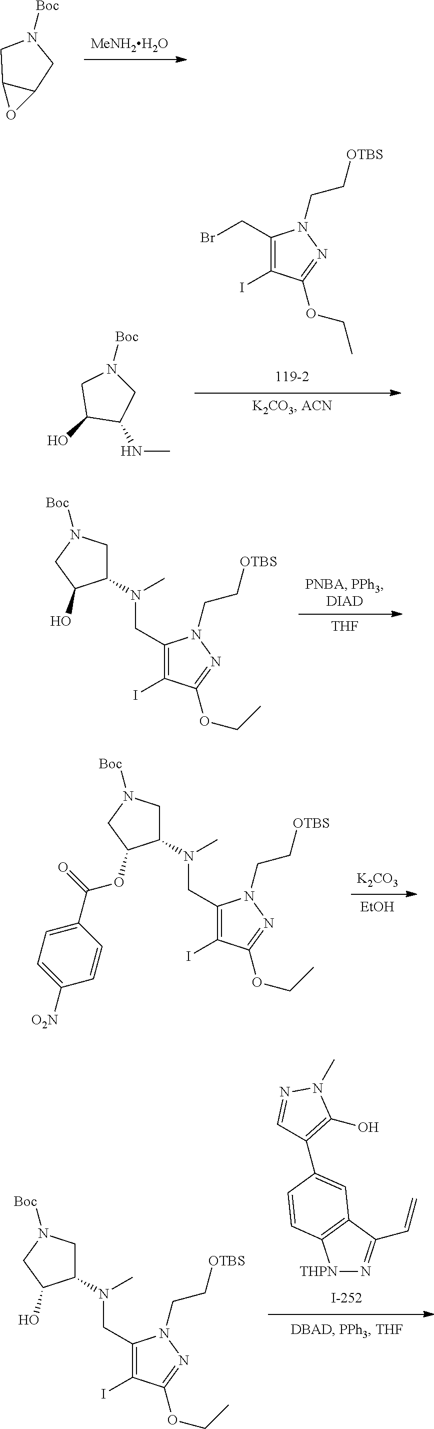

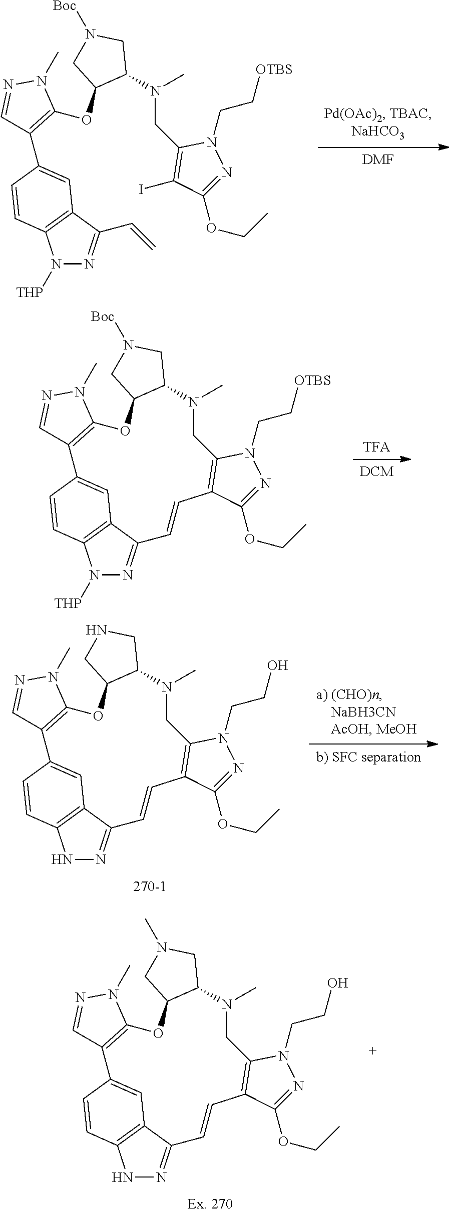

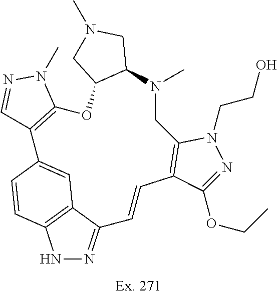 [SMILES: CCOc1nn(CCO)c2c1/C=C/c1n[nH]c3ccc(cc13)-c1cnn(C)c1O[C@@H]1CN(C)C[C@H]1N(C)C2]

Step 1. A solution of tert-butyl 6-oxa-3-azabicyclo[3.1.0]hexane-3-carboxylate (1.00 g, 5.40 mmol, 1 eq) in methanamine (13.0 g, 125 mmol, 23.2 eq) was stirred at 50° C. for 12 h. On completion, the mixture was quenched with water (150 mL) and extracted with ethyl acetate (80 mL×3), the combined organic phase was dried over anhydrous sodium sulfate, filtered and concentrated to give tert-butyl (3S,4S)-3-hydroxyl -4-(methylamino)pyrrolidine-1-carboxylate (1.00 g, 4.62 mmol, 85% yield) as off-white solid.

Step 2. A mixture of tert-butyl (3S,4S)-3-hydroxy-4-(methylamino)pyrrolidine-1-carboxylate (530 mg, 2.45 mmol, 1.2 eq) and 2-[5-(bromomethyl)-3-ethoxy-4-iodo -pyrazol-1-yl]ethoxy-tert-butyl-dimethyl-silane (1.00 g, 2.04 mmol, 1 eq) in ACN (10 mL) was added K₂CO₃ (847 mg, 6.13 mmol, 3 eq). The mixture was stirred at 40° C. for 1 h. On completion, the mixture was filtered and concentrated to give a residue. The residue was purified by column chromatography (SiO₂, PE:THF=1:0 to 3:1) to give tert-butyl (3S,4S)-3-[[2-[2-[tert-butyl(dimethyl) silyl]oxyethyl]-5-ethoxy-4-iodo-pyrazol-3-yl]methyl-methyl-amino]-4-hydroxy-pyrrolidine-1-carboxylate (1.40 g, crude) as colorless oil. LCMS: m/z 625.2 (M+1).

Step 3. A solution of tert-butyl (3S,4S)-3-[[2-[2-[tert-butyl(dimethyl)silyl]oxyethyl]-5-ethoxy-4-iodo-pyrazol-3-yl]methyl-methyl-amino]-4-hydroxy-pyrrolidine-1-carboxylate (1.30 g, 2.08 mmol, 1 eq), PPh₃ (1.09 g, 4.16 mmol, 2 eq) and 4-nitrobenzoic acid (521 mg, 3.12 mmol, 1.5 eq) in THE (19.5 mL) was stirred at 0° C. for 30 min. Then DIAD (841 mg, 4.16 mmol, 2 eq) was added to the mixture and stirred at 0° C. for 2 h. On completion, the mixture was concentrated to give a residue. The residue was purified by column chromatography (SiO₂, PE:THF=1:0 to 4:1) to give tert-butyl (3S,4R)-3-[[2-[2-[tert-butyl(dimethyl)silyl]oxyethyl]-5-ethoxy-4-iodo-pyrazol-3-yl]methyl-methyl-amino]-4-(4-nitrobenzoyl)oxy-pyrrolidine-1-carboxylate (1.44 g, 1.86 mmol, 89% yield) as yellow oil. LCMS: m/z 774.3 (M+1).

Step 4. To a solution of tert-butyl (3S,4R)-3-[[2-[2-[tert-butyl(dimethyl)silyl]oxyethyl]-5-ethoxy-4-iodo-pyrazol-3-yl]methyl-methyl-amino]-4-(4-nitrobenzoyl)oxy-pyrrolidine-1-carboxylate (1.34 g, 1.73 mmol, 1 eq) in EtOH (14 mL) was added K₂CO₃ (47.8 mg, 0.346 mmol, 0.2 eq). The mixture was stirred at 25° C. for 16 h. On completion, the mixture was filtered and concentrated to give a residue. The residue was purified by column chromatography (SiO₂, PE:THF=1:0 to 3:1) to give tert-butyl (3S,4R)-3-[[2-[2-[tert-butyl(dimethyl)silyl]oxyethyl]-5-ethoxy-4-iodo-pyrazol-3-yl]methyl-methyl-amino]-4-hydroxy-pyrrolidine-1-carboxylate (1.05 g, 1.68 mmol, 97% yield) as yellow oil. LCMS: m/z 625.1 (M+1).

Steps 5 through 7 were performed in a similar manner to steps 1 through 3 in General Method U to give racemic 270-1.

Step 8. The mixture of 2-[(8S,13R,20E)-18-ethoxy-5,13-dimethyl-7-oxa-4,5,10,13,16,17, 23,24-octazahexacyclo[20.5.2.0^{2,6}.0^{8,12}.0^{15,19}0.0^{25,29}]nonacosa-1(28),2(6),3,15(19), 17,20,22,25(29),26-nonaen-16-yl]ethanol (400 mg, 0.792 mmol, 1 eq) and CHO (161 mg, 4.76 mmol, 6 eq) in MeOH (5 mL) was added AcOH (47.6 mg, 0.792 mmol, 1 eq) adjust pH=6, the mixture was stirred at 25° C. for 0.3 h, and then NaBH₃CN (99.6 mg, 1.59 mmol, 2 eq) was added to the mixture. The mixture was stirred at 25° C. for 5 h. On completion, the mixture was quenched with water (0.5 mL) and concentrated to give a residue. The residue was purified by prep-HPLC (column: Waters Xbridge BEH C18 250*50 mm*10 um; mobile phase: [water(NH4HCO3)-ACN];gradient:17%-47% B over 10 min) to give desired compound (45.74 mg, 0.087 mmol, 11.0% yield, 99% purity) as white solid which was further separated by SFC (column: DAICEL CHIRALPAK IC (250 mm*30 mm,10 um);mobile phase: [C02-EtOH (0.1% NH3H2O)]; B %:40%, isocratic elution mode) to give arbitrarily assigned 2-|(4aS,7aS,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol (19.18 mg, 0.037 mmol, 48% yield, 99% purity) as off-white solid and 2-[(4aR,7aR,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol (18.47 mg, 0.035 mmol, 46% yield, 99.3% purity) as off-white solid.

### Preparation of {[(8aR,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile (Ex. 272) and {[(8aR,9R,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile (Ex. 273)

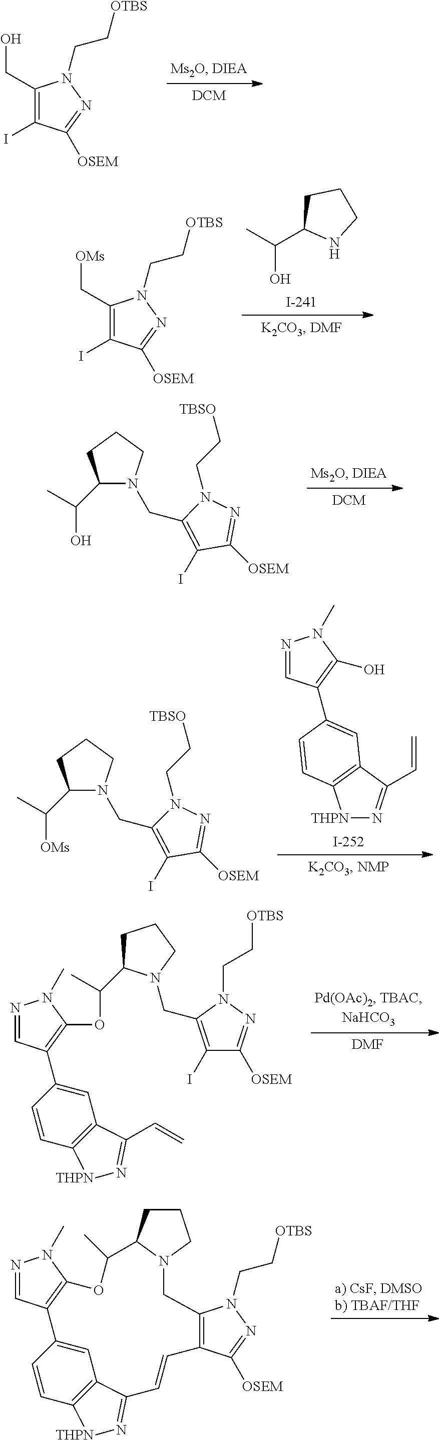

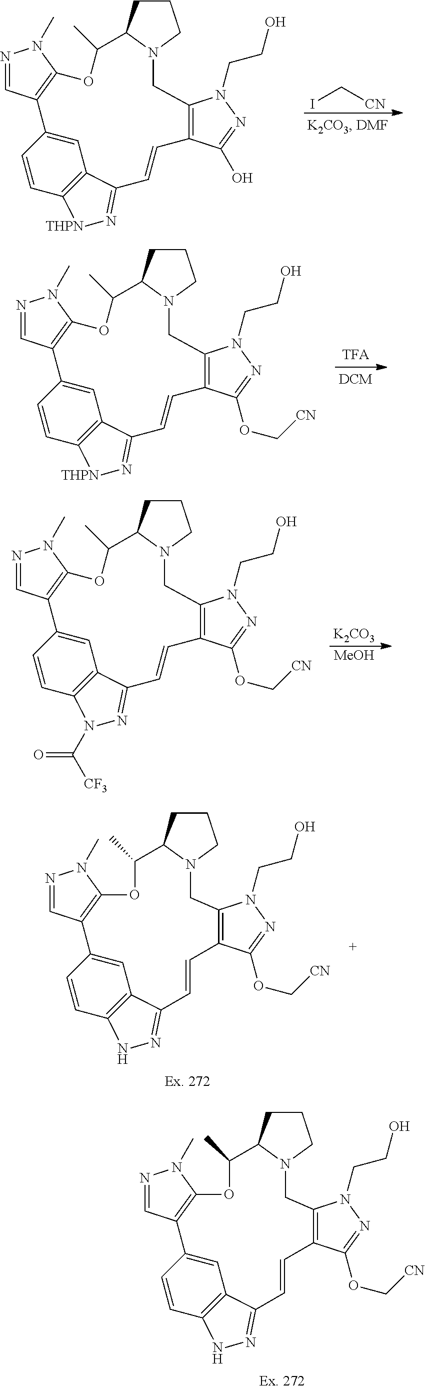

Step 1. To a solution of [2-[2-[tert-butyl(dimethyl)silyl]oxyethyl]-4-iodo-5-(2-trimethylsilylethoxymethoxy)pyrazol-3-yl]methanol (3.00 g, 5.68 mmol, 1 eq) from Ex. 186 in DCM (50 mL) was added methylsulfonyl methanesulfonate (1.48 g, 8.51 mmol, 1.5 eq) and DIEA (1.47 g, 11.3 mmol, 1.98 mL, 2 eq) at 0° C. The mixture was stirred at 25° C. for 1 h. On completion, the reaction mixture was concentrated under reduced pressure to give [2-[2-[tert-butyl(dimethyl)silyl]oxyethyl]-4-iodo-5-(2-trimethylsilylethoxymethoxy)pyrazol-3-yl]methyl methanesulfonate (6.40 g, crude) as brown solid. LCMS: m/z 607.0 (M+1).

Steps 2-5 were performed in a similar manner to steps 2-5 in General Method V.

Step 6. To a solution of tert-butyl-[2-[(9R,20E)-5,8-dimethyl-24-tetrahydropyran-2-yl-18-(2-trimethylsilylethoxymethoxy)-7-oxa-4,5,13,16,17,23,24-heptazahexacyclo[20.5.2.02, [01931]6.09,13.015,19.025,29]nonacosa-1(28),2(6),3,15(19),17,20,22,25(29),26-nonaen-16-yl]ethoxy]-dimethyl-silane (900 mg, 1.12 mmol, 1 eq) in DMSO (10 mL) was added CsF (850 mg, 5.60 mmol, 5 eq). The mixture was stirred at 25° C. for 1 h. On completion, the mixture was poured into H₂O (100 mL) and extracted with EA (50 mL*3), the combined organic phase was washed with H₂O (100 mL*2) and dried over anhydrous sodium sulfate, filtered and concentrated to give 2-[(9R,20E)-5,8-dimethyl-24-tetrahydropyran-2-yl-18-(2-trimethylsilylethoxymethoxy)-7-oxa-4,5,13,16,17,23,24-heptazahexacyclo [20.5.2.02,6.09,13.015,19.025,29]nonacosa-1(28),2(6),3,15(19),17,20,22,25(29),26-nonaen-16-yl]ethanol (650 mg, crude) as yellow solid. LCMS: m/z 690.3 (M+1).

Step 7. To a solution of 2-[(9R,20E)-5,8-dimethyl-24-tetrahydropyran-2-yl-18-(2-trimethylsilylethoxymethoxy)-7-oxa-4,5,13,16,17,23,24-heptazahexacyclo [20.5.2.02,6.09,13.015,19.025,29]nonacosa-1(28),2(6),3,15(19),17,20,22,25(29),26-nonaen-16-yl]ethanol (630 mg, 0.913 mmol, 1 eq) was added TBAF (4 M, 8 mL, 35 eq). The mixture was stirred at 70° C. for 12 h. On completion, the reaction mixture was diluted with water (50 ml) and was extracted with EA (30 mL*3). The combined organic layer was washed with brine (50 mL), dried over anh. Na₂SO₄, filtered and concentrated to give a residue. The residue was purified by column chromatography (SiO₂, DCM/MeOH=20:1 to 10:1) to give (9R,20E)-16-(2-hydroxyethyl)-5,8-dimethyl-24-tetrahydropyran-2-yl-7-oxa-4,5,13,16,17,23,24-heptazahexacyclo [20.5.2.02,6.09,13.015,19.025,29]nonacosa-1(28),2(6),3,15(19),17,20,22,25(29),26-nonaen-18-ol (377 mg, 0.673 mmol, 73% yield) as yellow solid. LCMS: m/z 560.2 (M+1).

Step 8. To a solution of (9R,20E)-16-(2-hydroxyethyl)-5,8-dimethyl-24-tetrahydropyran-2-yl-7-oxa-4,5,13,16,17,23,24-heptazahexacyclo[20.5.2.02,6.09,13.015,19.025,29]nonaco-sa-1(28),2(6),3,15(19),17,20,22,25(29),26-nonaen-18-ol (350 mg, 0.625 mmol, t eq) in DMF (5 mL) was added 2-iodoacetonitrile (125 mg, 0.750 mmol, 1.2 eq) and K₂CO₃ (172 mg, 1.25 mmol, 2 eq). The mixture was stirred at 80° C. for 1 h. On completion, the mixture was poured into H₂O (50 mL) and extracted with EA (30 mL*3), the combined organic phase was washed with H₂O (20 mL*3) and dried over anhydrous sodium sulfate, filtered and concentrated to give 2-[[(9R,20E)-16-(2-hydroxyethyl)-5,8-dimethyl-24-tetrahydropyran-2-yl-7-oxa-4,5,13,16,17,23,24-heptazahexacyclo[20.5.2.02,6.09,13.015,19.025,29]nonacosa-1(28),2(6),3,15(19),17,20,22,25(29),26-nonaen-18-yl]oxy]acetonitrile (350 mg, crude) as green solid. LCMS: m/z 599.2 (M+1).

Step 9 was performed in a similar manner to step 6 in General Method V.

Step 10. To a solution of 2-[[(9R,20E)-16-(2-hydroxyethyl)-5,8-dimethyl-24-(2,2,2-trifluoroacetyl)-7-oxa-4,5,13,16,17,23,24-heptazahexacyclo[20.5.2.02,6.09,13.015,19.025,29]nonacosa-1(28),2(6),3,15(19),17,20,22,25(29),26-nonaen-18-yl]oxy]acetonitrile (350 mg, 0.573 mmol, 1 eq) in MeOH (6 mL) was added K₂CO₃ (396 mg, 2.87 mmol, 5 eq). The mixture was stirred at 25° C. for 1 h. On completion, the mixture was filtered and the filtrate was concentrated in vacuo to give a residue. The residue was purified by prep-HPLC purification (column: Welch Xtimate C18 150*25 mm*5 um; mobile phase: [water(TFA)-ACN I; gradient:12%-42% B over min) to give arbitrarily assigned {|(8aR,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile (32.3 mg, 0.063 mmol, 11% yield, 99.7% purity) as yellow solid and {[(8aR,9R,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile (38.1 mg, 0.072 mmol, 13% yield, 97% purity) as yellow solid.

### Preparation of 2-{(10S,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 274) and 2-{(10R,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 275)

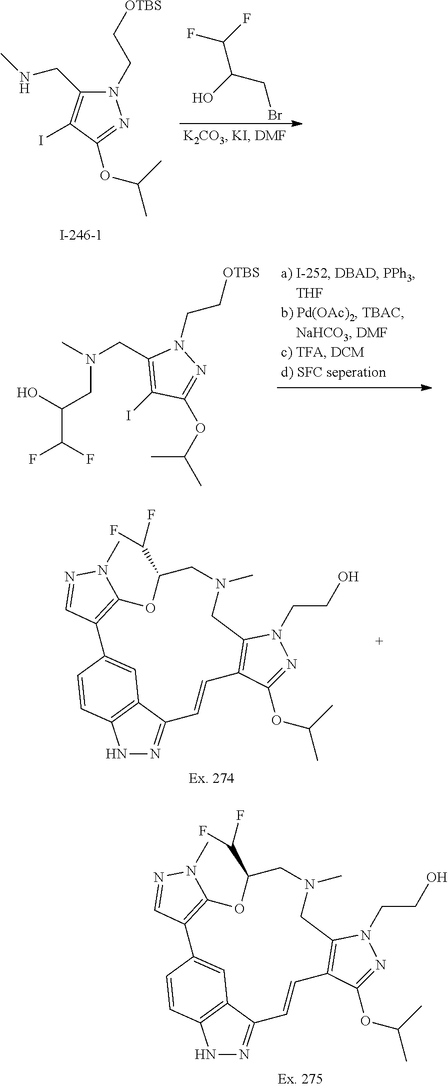

Step 1. To a solution of 1-[2-[2-[tert-butyl(dimethyl)silyl]oxyethyl]-4-iodo-5-isopropoxy-pyrazol-3-yl]-N-methylmethanamine (1.00 g, 2.21 mmol, 1 eq) in DMF (10 mL) was added KI (366 mg, 2.21 mmol, 1 eq) 3-bromo-1,1-difluoro-propan-2-ol (463 mg, 2.65 mmol, 1.2 eq) and K₂CO₃ (609 mg, 4.41 mmol, 2 eq). The mixture was stirred at 80° C. for 16 hours. On completion, the reaction mixture was diluted with H₂O (20 ml) and extracted with EA (3 * 20 mL). The combined organic layers were washed with brine (2 * 20 mL), dried over by anhydrous Na₂SO₄, filtered and concentrated in vacuo to give a residue. And then the residue was purified by column chromatography (SiO₂, Petroleum ether/Ethyl acetate=1/0 to 0.5/1) to give 3-[[2-[2-[tert-butyl(dimethyl)silyl]oxyethyl]-4-iodo-5-isopropoxy-pyrazol-3-yl]methyl-methyl-amino]-1,1-difluoro-propan-2-ol (288 mg, 0.526 mmol, 24% yield) as yellow oil. LCMS: m/z 548.1 (M+1).

Steps 2-4 were performed in a similar manner to General Method U followed by SFC separation to provide arbitrarily assigned Ex. 274 and Ex. 275.

### Preparation of 2-{(10S,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol (Ex. 276)

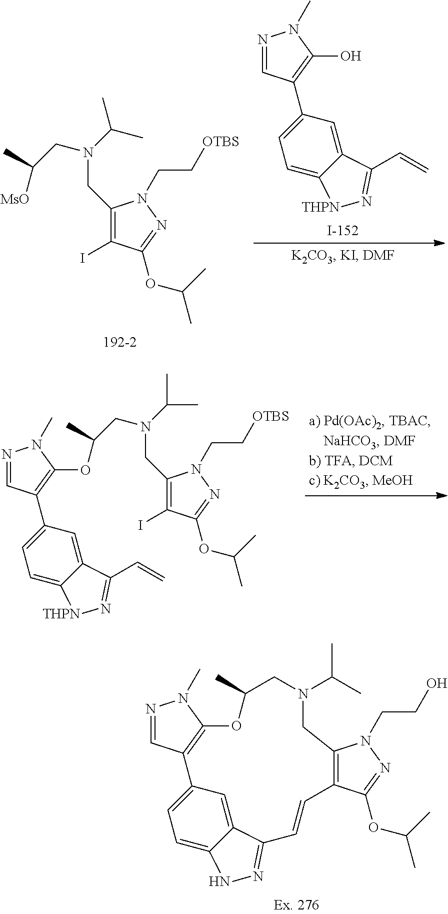

Step 1. To a solution of 2-methyl-4-(1-tetrahydropyran-2-yl-3-vinyl-indazol-5-yl)pyrazol-3-ol (326 mg, 1.01 mmol, 1 eq) and [(1S)-2-[[2-[2-[tert-butyl(dimethyl)silyl]oxyethyl]-4-iodo-5-isopropoxy-pyrazol-3-yl]methyl-isopropyl-amino]-1-methyl-ethyl]methanesulfonate (745 mg, 1.21 mmol, 1.2 eq) in DMF (20 mL) was added K₂CO₃ (417 mg, 3.02 mmol, 3 eq). The mixture was stirred at 60° C. or 1 h. On completion, the mixture was quenched with water (40 mL) and extracted with ethyl acetate (25 mL×3), the combined organic phase was dried over anhydrous sodium sulfate, filtered and concentrated to give a residue. The residue was purified by column chromatography (SiO₂, PE:EA=1:0 to 2:1) to give 2S)—N-[[2-[2-[tert-butyl(dimethyl)silyl]oxyethyl]-4-iodo-5-isopropoxy-pyrazol-3-yl]methyl]-N-isopropyl-2-[2-methyl-4-(1-tetrahydropyran-2-yl-3-vinyl-indazol-5-yl)pyrazol-3-yl]oxy-propan-1-amine (543 mg, 0.642 mmol, 64% yield) as a yellow solid. LCMS: m/z 846.4 (M+1).

Steps 2-4 were performed in a similar manner to steps 5, 9, and 10 in Example 272 to give 276 (19.39 mg, 0.037 mmol, 38.3% yield) as a white solid.

### General Method Y: Preparation of 2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 277)

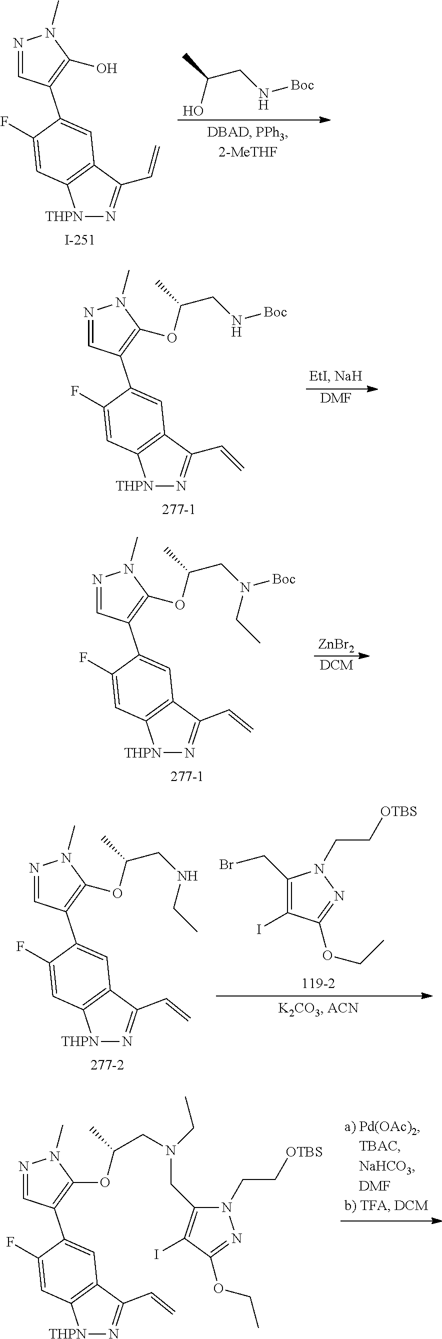

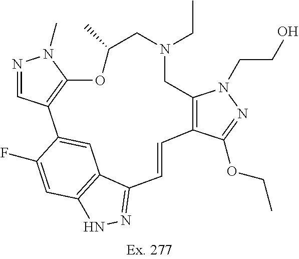 [SMILES: CCOc1nn(CCO)c2c1/C=C/c1n[nH]c3cc(F)c(cc13)-c1cnn(C)c1O[C@H](C)CN(CC)C2]

Step 1. A mixture of 4-(6-fluoro-1-tetrahydropyran-2-yl-3-vinyl-indazol-5-yl)-2-methyl-pyrazol-3-ol (2.00 g, 5.84 mmol, 1 eq), tert-butyl N-[(2S)-2-hydroxypropyl]carbamate (2.05 g, 11.6 mmol, 2 eq), DBAD (2.96 g, 12.8 mmol, 2.2 eq), PPh₃ (3.37 g, 12.8 mmol, 2.2 eq) was degassed and purged with N₂ for 3 times, then added 2-MeTHF (20 mL) was degassed and purged with N₂ for 3 times again, and then the mixture was stirred at 25° C. for 2 h under N₂ atmosphere. On completion, the reaction was concentrated under vacuum. The residue was purified by column chromatography (SiO₂, Petroleum ether/Ethyl acetate=3:1 to 1:1) to give tert-butyl N-[(2R)-2-[4-(6-fluoro-1-tetrahydropyran-2-yl-3-vinyl-indazol-5-yl)-2-methyl-pyrazol-3-yl]oxypropyl]carbamate (7.10 g, 5.68 mmol, 97% yield, 40% purity) as yellow solid. LCMS: m/z 500.1 (M+1).

Step 2. To a solution of tert-butyl N-[(2R)-2-[4-(6-fluoro-1-tetrahydropyran-2-yl-3-vinyl-indazol-5-yl)-2-methyl-pyrazol-3-yl]oxypropyl]carbamate (6.60 g, 5.28 mmol, 1 eq) in DMF (20 mL) was added NaH (845 mg, 21.1 mmol, 60% purity, 4 eq) at 0° C. The mixture was stirred at 25° C. for 0.5 h, then was added iodoethane (2.47 g, 15.8 mmol, 1.27 mL, 3 eq) at 25° C., the mixture was stirred at 25° C. for 6 h. On completion, the mixture was poured into H₂O (200 mL) and extracted with EA (60 mL*3). The combined organic phase was washed with H₂O (100 mL*3) and dried over anhydrous sodium sulfate, filtered and concentrated to give a residue. The residue was purified by column chromatography (SiO₂, Petroleum ether/Ethyl acetate=5:1 to 3:1) to give tert-butyl N-ethyl-N-[(2R)-2-[4-(6-fluoro-1-tetrahydropyran-2-yl-3-vinyl-indazol-5-yl)-2-methyl-pyrazol-3-yl]oxypropyl]carbamate (2.40 g, 4.55 mmol, 86% yield) as colorless oil. ¹H NMR (400 MHz, CDCl₃) δ=7.97 (dd, J=2.0, 6.8 Hz, 1H), 7.58 (br s, 1H), 7.31 (br d, J=10.4 Hz, 1H), 7.01 (dd, J=11.6, 18.0 Hz, 1H), 6.08 (d, J=18.0 Hz, 1H), 5.62 (dd, J=2.4, 9.4 Hz, 1H), 5.55 (d, J=11.6 Hz, 1H), 4.30-4.12 (m, 1H), 4.07 (br d, J=11.2 Hz, 1H), 3.75 (s, 4H), 3.51-2.99 (m, 4H), 2.59-2.46 (m, 1H), 2.22-2.11 (m, 1H), 2.06 (br dd, J=2.8, 13.2 Hz, 1H), 1.77 (br t, J=8.4 Hz, 2H), 1.71-1.60 (m, 1H), 1.45-1.32 (m, 9H), 0.96 (br s, 6H); LCMS: m/z 528.1 (M+1).

Step 3. To a solution of tert-butyl N-ethyl-N-[(2R)-2-[4-(6-fluoro-1-tetrahydropyran-2-yl-3-vinyl-indazol-5-yl)-2-methyl-pyrazol-3-yl]oxypropyl]carbamate (2.00 g, 3.79 mmol, 1 eq) in DCM (20 mL) was added ZnBr₂ (2.56 g, 11.3 mmol, 3 eq). The mixture was stirred at 25° C. for 12 hr. On completion, the mixture was concentrated in vacuum to give (2R)—N-ethyl-2-[4-(6-fluoro-1-tetrahydropyran-2-yl-3-vinyl-indazol-5-yl)-2-methyl-pyrazol-3-yl]oxy-propan-1-amine (4.20 g, 3.73 mmol, 98% yield, 38% purity) as white solid. LCMS: m/z 428.1 (M+1).

Step 4. To a solution of (2R)—N-ethyl-2-[4-(6-fluoro-1-tetrahydropyran-2-yl-3-vinyl-indazol-5-yl)-2-methyl-pyrazol-3-yl]oxy-propan-1-anine (500 mg, 1.17 mmol, 1 eq) in DMF (5 mL) was added K₂CO₃ (485 mg, 3.51 mmol, 3 eq) and 2-[5-(bromomethyl)-3-ethoxy-4-iodo-pyrazol-1-yl]ethoxy-tert-butyl-dimethyl-silane (763 mg, 1.17 mmol, 1 eq). On completion, the mixture was stirred at 80° C. for 1 h. The mixture was poured into H₂O (50 mL) and extracted with ethyl acetate (30 mL×3), the combined organic phase was washed with H₂O (50 mL×3) and dried over anhydrous sodium sulfate, filtered and concentrated to give a residue. The residue was purified by column chromatography (SiO₂, Petroleum ether/THF=1/0 to 92/8) to give (2R)—N-[[2-[2-[tert-butyl(dimethyl)silyl]oxyethyl]-5-ethoxy-4-iodo-pyrazol-3-yl]methyl]-N-ethyl-2-[4-(6-fluoro-1-tetrahydropyran-2-yl-3-vinyl-indazol-5-yl)-2-methyl-pyrazol-3-yl]oxy-propan-1-amine (0.508 g, 0.608 mmol, 52% yield) as a light yellow oil. LCMS: m/z 836.1 (M+1).

Steps 5 and 6 were performed in a similar manner to steps 3 and 4 in General Method T.

### Preparation of (4aS,7aS,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine (Ex. 278) and (4aR,7aR,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine (Ex. 279)

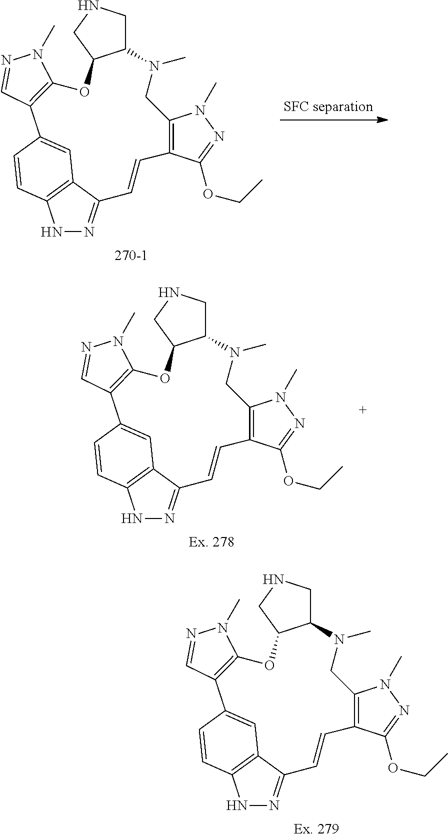

SFC separation of racemic 270-1 gave arbitrarily assigned Ex. 278 and Ex. 279.

### Preparation of (2S)-1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol (Ex. 280)

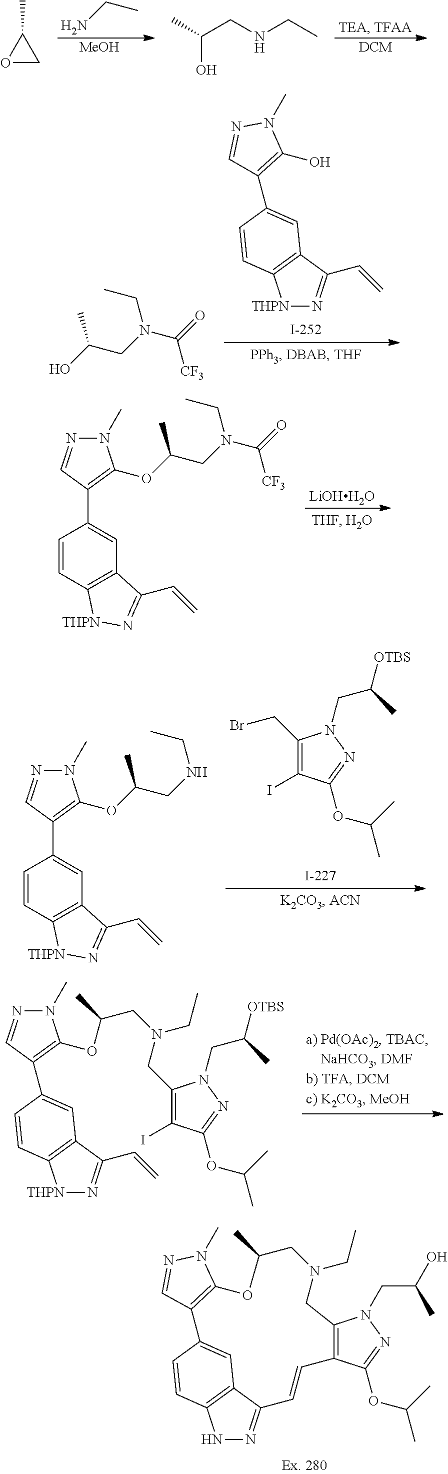

Step 1. To a solution of (2R)-2-methyloxirane (10 g, 172.18 mmol, 1 eq) in MeOH (100 mL) was added ethanamine (2 M, 258 mL, 3 eq). The mixture was stirred at 25° C. for 12 h. On completion, the mixture was concentrated to give (2R)-1-(ethylamino) propan-2-ol (8.13 g, 78.8 mmol, 46% yield) as a brown solid. ¹H NMR (400 MHz, CDCl₃) δ=3.84-3.70 (m, 1H), 2.74-2.55 (m, 3H), 2.45-2.34 (m, 2H), 1.16-1.05 (m, 6H).

Step 2. To a solution of (2R)-1-(ethylamino)propan-2-ol (8.13 g, 78.8 mmol, 1 eq) in DCM (80 mL) was added (2,2,2-trifluoroacetyl) 2,2,2-trifluoroacetate (24.8 g, 118 mmol, 16.4 mL, 1.5 eq) and TEA (39.9 g, 394 mmol, 5 eq) at 0° C., then the mixture was stirred at 0° C. for 1 h. On completion, the reaction mixture was partitioned between DCM (100 mL×3) and water (100 mL), the combined organic phase was dried over anhydrous sodium sulfate, filtered and concentrated to give a residue. The residue was purified by column chromatography (SiO₂, PE:THF=1:1 to 1:1) to give N-ethyl-2,2,2-trifluoro-N-[(2R)-2-hydroxypropyl]acetamide (7.92 g, 39.8 mmol, 50% yield) as a white solid. LCMS: m/z 200.1 (M+1).

Step 3. A mixture of 2-methyl-4-(1-tetrahydropyran-2-yl-3-vinyl-indazol-5-yl)pyrazol-3-ol (700 mg, 2.16 mmol, 1 eq), N-ethyl-2,2,2-trifluoro-N-[(2R)-2-hydroxypropyl]acetamide (429 mg, 2.16 mmol, 1 eq), PPh₃ (1.13 g, 4.32 mmol, 2 eq) and DBAD (745 mg, 3.24 mmol, 1.5 eq) was degassed and purged with N₂ for 3 times, then THF (11 mL) was added. The mixture was stirred at 25° C. for 1 h. On completion, the mixture was concentrated to give a residue. The residue was purified by column chromatography (SiO₂, PE:THF=1:0 to 2:1) to give N-ethyl-2,2,2-trifluoro-N-[(2S)-2-[2-methyl-4-(1-tetrahydropyran-2-yl-3-vinyl-indazol-5-yl) pyrazol-3-yl]oxypropyl]acetamide (920 mg, 1.82 mmol, 84% yield) as a yellow oil. LCMS: m/z 506.0 (M+1).

Step 4. To a solution of N-ethyl-2,2,2-trifluoro-N-[(2S)-2-[2-methyl-4-(1-tetrahydropyran-2-yl-3-vinyl-indazol-5-yl)pyrazol-3-yl]oxypropyl]acetamide (870 mg, 1.72 mmol, 1 eq) in THF (8.7 mL) and H₂O (1.74 mL) was added LiOH·H₂O (361 mg, 8.60 mmol, 5 eq). The mixture was stirred at 25° C. for 12 h. On completion, the mixture was quenched with water (20 mL) and extracted with ethyl acetate (15 mL×3), the combined organic phase was dried over anhydrous sodium sulfate, filtered and concentrated to give a residue. The residue was purified by column chromatography (SiO₂, Dichloromethane:Methanol=1:0 to 10:1) to give (2S)—N-ethyl-2-[2-methyl-4-(1-tetrahydropyran-2-yl-3-vinyl-indazol-5-yl)pyrazol-3-yl]oxy-propan-1-amine (650 mg, 1.59 mmol, 92% yield) as a yellow oil. LCMS: m/z 410.2 (M+1).

Step 5. To a solution of (2S)—N-ethyl-2-[2-methyl-4-(1-tetrahydropyran-2-yl-3-vinyl-indazol-5-yl)pyrazol-3-yl]oxy-propan-1-amine (316 mg, 0.773 mmol, 1 eq) in ACN (3.5 mL) was added |(1S)-2-|5-(bromomethyl)-4-iodo-3-isopropoxy-pyrazol-1-yl|-1-methyl-ethoxy]-tert-butyl-dimethyl-silane (400 mg, 0.773 mmol, 1 eq) and K₂CO₃ (320 mg, 2.32 mmol, 3 eq). The mixture was stirred at 60° C. for 2 h. On completion, the mixture was filtered and concentrated to give a residue. The residue was purified by column chromatography (SiO₂, PE:THF=1:0 to 4:1) to give (2S)—N-[[2-[(2S)-2-[tert-butyl(dimethyl)silyl]oxypropyl]-4-iodo-5-isopropoxy-pyrazol-3-yl]methyl]-N-ethyl-2-[2-methyl-4-(1-tetrahydropyran-2-yl-3-vinyl-indazol-5-yl)pyrazol-3-yl]oxy-propan-1-amine (520 mg, 0.614 mmol, 79% yield) as a colorless oil. LCMS: m/z 846.5 (M+1).

Steps 6-8 were performed in a similar manner to steps 5, 9, and 10 in Example 272 to give 280 (44.3 mg, 0.078 mmol, 48% yield, 99% purity, FA) as a yellow solid.

Examples 281 and 282 were arbitrarily assigned from synthesis following General Method V.

Examples 283 and 284 were arbitrarily assigned from synthesis following General Method V.

### Preparation of {[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile (Ex. 285)

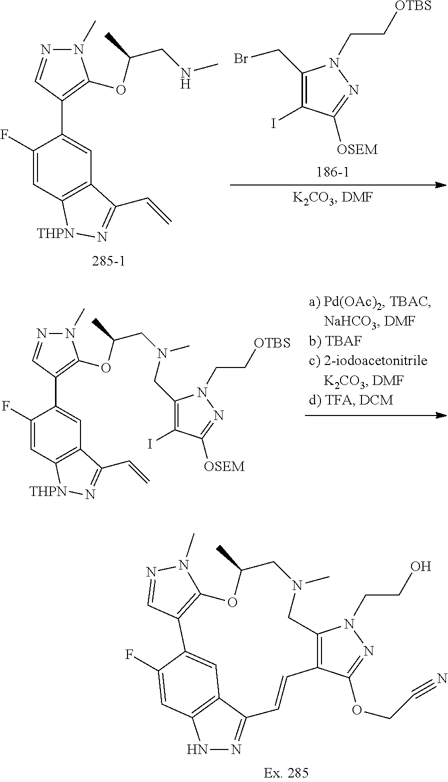

Starting material 285-1 was made following the procedures of General Method Y steps 1 through 3.

Step 1. To a solution of (2S)-2-[4-(6-fluoro-1-tetrahydropyran-2-yl-3-vinyl-indazol-5-yl)-2-methyl-pyrazol-3-yl]oxy-N-methyl-propan-1-amine (830 mg, 2.01 mmol, 1 eq) in DMF (10 mL) was added K₂CO₃ (832 mg, 6.02 mmol, 3 eq) and 2-[[5-(bromomethyl)-1-[2-[tert-butyl(dimethyl)silyl]oxyethyl]-4-iodo-pyrazol-3-yl]oxymethoxy]ethyl-trimethyl-silane (1.54 g, 2.61 mmol, 1.3 eq). The mixture was stirred at 80° C. for 16 h. On completion, the reaction mixture was diluted with H₂O (30 mL) and extracted with EA (20 mL*3). The combined organic layers were washed with H₂O (40 mL*3), dried over Na₂SO₄, filtered and concentrated under reduced pressure to give a residue. The residue was purified by column chromatography (SiO₂, Petroleum ether/Ethyl acetate=1/0 to 0/1) to give (2S)—N-[[2-[2-[tert-butyl(dimethyl)silyl]oxyethyl]-4-iodo-5-(2-trimethylsilylethoxymethoxy)pyrazol-3-yl]methyl]-2-[4-(6-fluoro-1-tetrahydropyran-2-yl-3-vinyl-indazol-5-yl)-2-methyl-pyrazol-3-yl]oxy-N-methyl-propan-1-amine (630 mg, 0.682 mmol, 34% yield) as colorless oil. LCMS: m/z 924.6 (M+1).

Steps 2 through 5 were performed in a similar manner to steps 4 through 7 in General Method X.

Example 286 was prepared using similar procedures to Example 285.

Example 287 was prepared using similar procedures to Example 280.

Example 288 was prepared using similar procedures to General Method Y.

### Preparation of (2S)-2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol (Ex. 289)

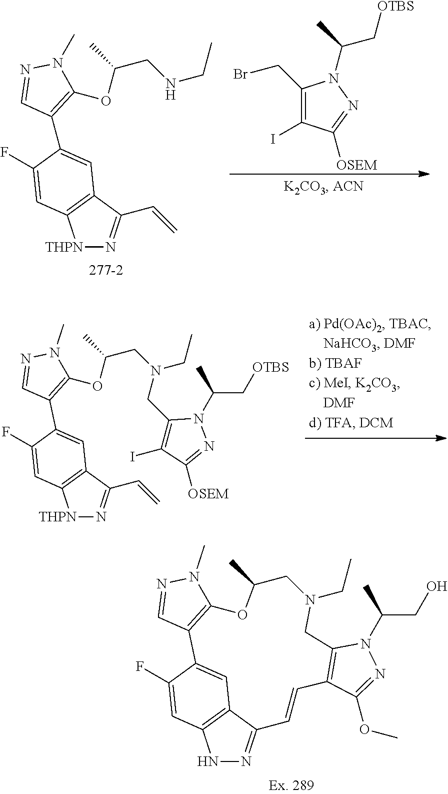

Step 1. A solution of (2S)—N-ethyl-2-[4-(6-fluoro-1-tetrahydropyran-2-yl-3-vinyl-indazol-5-yl)-2-methyl-pyrazol-3-yl]oxy-propan-1-amine (250 mg, 0.585 mmol, 1 eq), 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane (425 mg, 0.702 mmol, 1.2 eq) from Ex. 214 and K₂CO₃ (242 mg, 1.75 mmol, 3 eq) in ACN (6 mL) was stirred at 60° C. for 2 h. On completion, the reaction mixture was concentrated in vacuo. The residue was purified by column chromatography on silica gel (4 g silica gel, EA in Petroleum ether from 0% to 100%) to give (2S)—N-[[2-[(1S)-2-[tert-butyl(dimethyl)silyl]oxy-1-methyl-ethyl]-4-iodo-5-(2-trimethylsilylethoxymethoxy) pyrazol-3-yl]methyl]-N-ethyl-2-[4-(6-fluoro-1-tetrahydropyran-2-yl-3-vinyl-indazol-5-yl)-2-methyl-pyrazol-3-yl]oxy-propan-1-amine (400 mg, 0.298 mmol, 51% yield, 71% purity) as a yellow solid. LCMS: m/z 952.5 (M+1).

Steps 2 through 5 were performed in a similar manner to steps 4 through 7 in General Method X.

### Preparation of (10S,17E)-16-ethoxy-12-ethyl-14-(2-hydroxyethyl)-8,10-dimethyl-2,10,11,12,13,14-hexahydro-8H-3,5-ethenotripyrazolo[3,4-f:3′,4′-j:4″,3″-n][1,4]oxazacyclopentadecine-6-carbonitrile (Ex. 290)

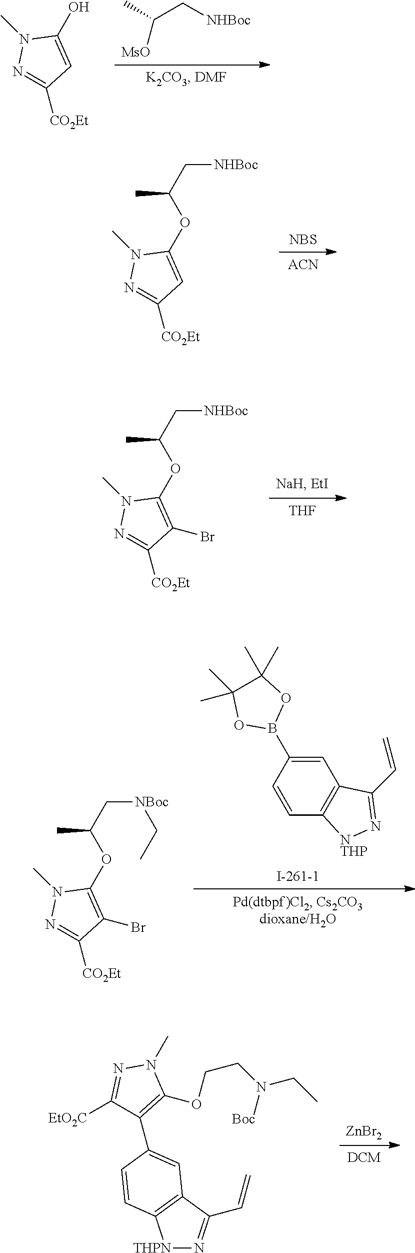

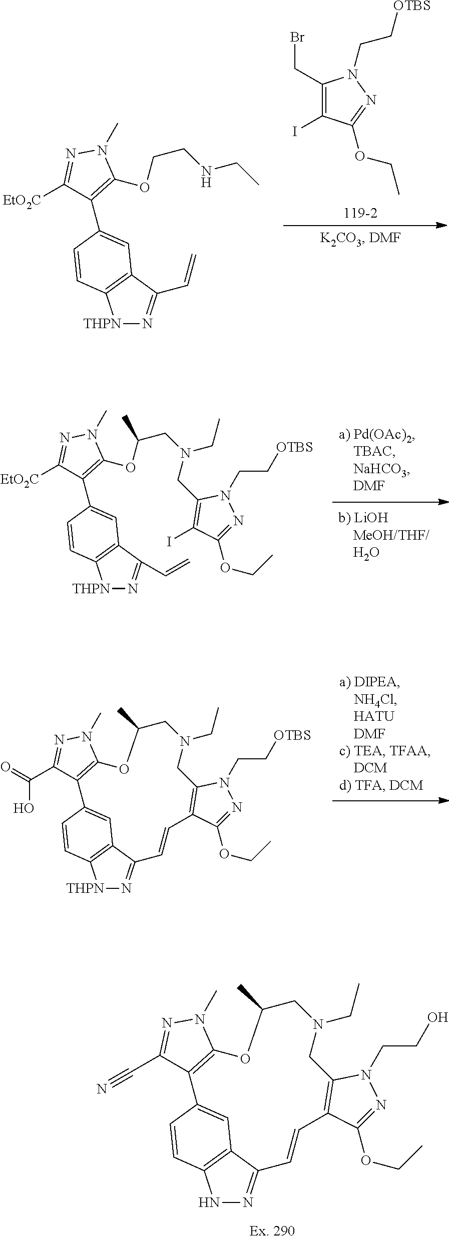

Steps 1-3 were conducted according to General Method M steps 2-4.

Step 4. A mixture of ethyl 4-bromo-5-[(1S)-2-[tert-butoxycarbonyl(ethyl)amino]-1-methyl-ethoxy]-1-methyl-pyrazole-3-carboxylate (1.30 g, 2.99 mmol, 1 eq), 1-tetrahydropyran-2-yl-5-(4,4,5,5-tetramethyl-1,3,2-dioxaborolan-2-yl)-3-vinyl-indazole (1.17 g, 3.29 mmol, 1.1 eq), ditert-butyl(cyclopentyl)phosphane; dichloropalladium; iron (195 mg, 0.299 mmol, 0.1 eq), Cs₂CO₃ (2.93 g, 8.98 mmol, 3 eq) in dioxane (15 mL) and H₂O (3 mL) was degassed and purged with N₂ for 3 times, and then the mixture was stirred at 80° C. for 1 hour under N₂ atmosphere. On completion, the mixture was quenched with water (50 mL) and extracted with ethyl acetate (45 mL*3), the combined organic phase was dried over anhydrous sodium sulfate, filtered and concentrated to give a residue. The residue was purified by column chromatography (SiO₂, Petroleum ether/THF=1:0 to 1:1) to give ethyl 5-[(1S)-2-[tert-butoxycarbonyl(ethyl)amino]-1-methyl-ethoxy]-1-methyl-4-(1-tetrahydropyran-2-yl-3-vinyl-indazol-5-yl) pyrazole-3-carboxylate (700 mg, 1.20 mmol, 40% yield) as an orange solid. LCMS: m/z 582.3 (M+1).

Steps 5-6 were conducted according to General Method Q steps 5 and 6.

Step 7. A mixture of ethyl 5-[(1S)-2-[[2-[2-[tert-butyl(dimethyl)silyl]oxyethyl]-5-ethoxy-4-iodo-pyrazol-3-yl]methyl-ethyl-amino]-1-methyl-ethoxy]-1-methyl-4-(1-tetrahydropyran-2-yl-3-vinyl-indazol-5-yl) pyrazole-3-carboxylate (300 mg, 0.337 mmol, 1 eq), Pd(OAc)₂ (15.1 mg, 0.067 mmol, 0.2 eq), TBAC (93.6 mg, 0.337 mmol, 1 eq) and NaHCO₃ (70.8 mg, 0.842 mmol, 2.5 eq) in DMF (5 mL) was degassed and purged with N₂ for 3 times, and then the mixture was stirred at 120° C. for 10 minutes under N₂ atmosphere. On completion, the mixture was quenched with water (20 mL) and extracted with ethyl acetate (25 mL*3), the combined organic phase was dried over anhydrous sodium sulfate, filtered and concentrated to give a residue. The residue was purified by column chromatography (SiO₂, Petroleum ether/Ethyl acetate=1:0 to 1:1) to give ethyl (8S,17E)-13-[2-[tert-butyl(dimethyl)silyl]oxyethyl]-15-ethoxy-10-ethyl-5,8-dimethyl-21-tetrahydropyran-2-yl-7-oxa-4,5,10,13, 14,20,21- heptazapentacyclo [17.5.2.02,6.012,16.022,26]hexacosa-1(25),2(6),3, 12(16),14,17,19,22(26),23-nonaene-3-carboxylate (200 mg, 0.262 mmol, 77% yield) as a white solid. LCMS: m/z 762.2 (M+1).

Step 8. To a solution of ethyl (8S,17E)-13-[2-[tert-butyl(dimethyl)silyl]oxyethyl]-15-ethoxy-10-ethyl-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19, 22(26),23-nonaene-3-carboxylate (200 mg, 0.262 mmol, 1 eq) in MeOH (1.5 mL), H₂O (0.5 mL) and THF (1.5 mL) was added LiOH·H₂O (33.0 mg, 0.787 mmol, 3 eq). The mixture was stirred at 25° C. for 1 hour. On completion, the mixture was filtered and concentrated to give a residue. The residue was purified by reversed-phase HPLC (Neutral condition) to give (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaene-3-carboxylic acid (50.0 mg, 0.081 mmol, 31% yield) as a yellow solid. LCMS: m/z 620.1 (M+1).

Step 9. To a solution of (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012, 16.022,26]hexacosa-1(25),2(6),3,12(16),14,17,19,22(26),23-nonaene-3-carboxylic acid (30.0 mg, 0.048 mmol, 1 eq) and NH4C1 (25.9 mg, 0.484 mmol, 10 eq) in DMF (0.5 mL) was added DIEA (31.2 mg, 0.242 mmol, 5 eq) and HATU (22.0 mg, 0.058 mmol, 1.2 eq). The mixture was stirred at 25° C. for 0.1 hour. On completion, the mixture was filtered and concentrated to give a residue. The residue was purified by reversed-phase HPLC (0.1% FA condition) to give (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaene-3-carboxamide (15.0 mg, 0.024 mmol, 50% yield) as a white solid. LCMS: m/z 619.3 (M+1).

Step 10. To a solution of (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaene-3-carboxamide (15.0 mg, 0.024 mmol, 1 eq) in CHCl₃ (0.5 mL) was added TEA (8.34 mg, 0.082 mmol, 3.4 eq) and TFAA (8.66 mg, 0.041 mmol, 1.7 eq). The mixture was stirred at 0° C. for 1 hour. On completion, the mixture was quenched with water (5 mL) and extracted with ethyl acetate (3 mL*3), the combined organic phase was dried over anhydrous sodium sulfate, filtered and concentrated to give (8S,17E)-15-ethoxy-10-ethyl-13-(2-hydroxyethyl)-5,8-dimethyl-21-tetrahydropyran-2-yl-7-oxa-4,5,10,13,14, 20,21-heptazapentacyclo [17.5.2.02,6.012,16.022,26]hexacosa-1(25),2(6),3,12(16),14, 17,19,22(26),23-nonaene-3-carbonitrile (14.0 mg, 0.023 mmol, 96% yield) as a yellow solid. LCMS: m/z 601.2 (M+1).

Step 11. To a solution of (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012, 16.022,26]hexacosa-1(25),2(6),3,12(16),14,17,19,22(26),23-nonaene-3-carbonitrile (14.0 mg, 0.023 mmol, 1 eq) in DCM (0.5 mL) was added TFA (26.5 mg, 0.233 mmol, 10 eq). The mixture was stirred at 25° C. for 0.1 hours. On completion, the mixture was concentrated to give a residue. The residue was purified by prep-HPLC (column: Phenomenex C₁₈ 150*25 mm*10 um; mobile phase: [water(NH₄HCO₃)-ACN]; gradient:26%-56% B over 10 min) to give (10S,17E)-16-ethoxy-12-ethyl-14-(2-hydroxyethyl)-8,10-dimethyl-2,10,11,12,13,14-hexahydro-8H-3,5-ethenotripyrazolo[3,4-f:3′,4′-j:4″,3″-n][1,4]oxazacyclopentadecine-6-carbonitrile (2.38 mg, 0.004 mmol, 19% yield, 94% purity) as a yellow solid.

### Preparation of 1-[(17E)-16-ethyl-8,15-dimethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one (Ex. 291)

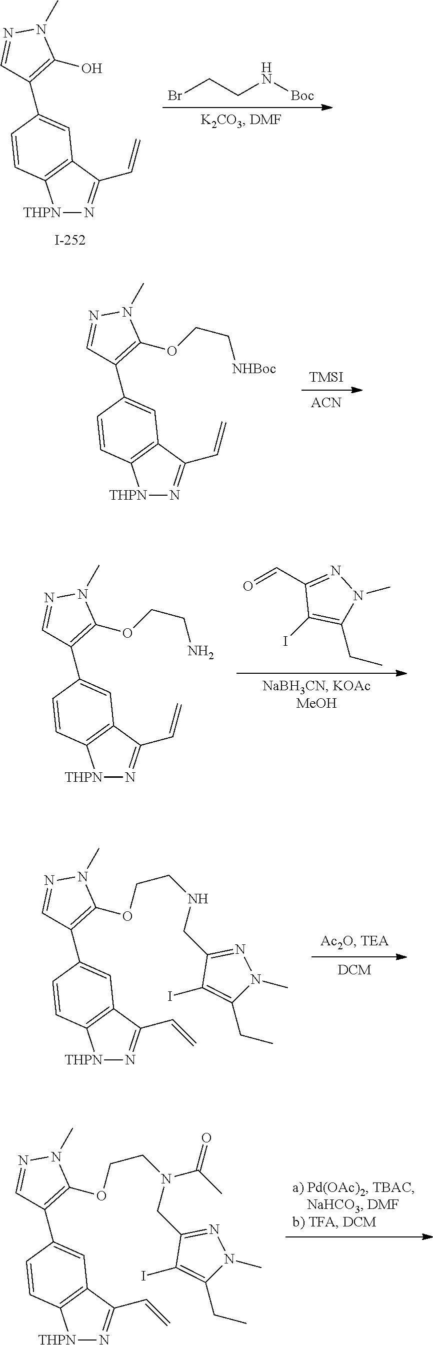

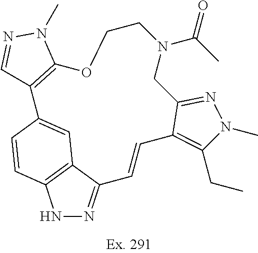 [SMILES: CCc1c2c(nn1C)CN(C(C)=O)CCOc1c(cnn1C)-c1ccc3[nH]nc(c3c1)/C=C/2]

Step 1. To a solution of 2-methyl-4-(1-tetrahydropyran-2-yl-3-vinyl-indazol-5-yl)pyrazol-3-ol (4.00 g, 12.3 mmol, 1 eq) and tert-butyl N-(2-bromoethyl)carbamate (4.15 g, 18.5 mmol, 1.5 eq) in DMF (40 mL) was added K₂CO₃ (5.11 g, 36.9 mmol, 3 eq). The mixture was stirred at 80° C. for 1 hr. On completion, the mixture was diluted with water (60 mL) and extracted with ethyl acetate (60 mL×3), the combined organic phase was dried over anhydrous sodium sulfate, filtered and concentrated to give a residue. The residue was purified by column chromatography (SiO₂, Petroleum ether/Ethyl acetate=1:1 to 1:1) to give tert-butyl N-[2-[2-methyl-4-(1-tetrahydropyran-2-yl-3-vinyl-indazol-5-yl)pyrazol-3-yl]oxyethyl]carbamate (3.70 g, 7.68 mmol, 62% yield, 97% purity) as a yellow oil. LCMS: m/z 468.1 (M+1).

Step 2. To a solution of tert-butyl N-[2-[2-methyl-4-(1-tetrahydropyran-2-yl-3-vinyl-indazol-5-yl)pyrazol-3-yl]oxyethyl]carbamate (3.20 g, 6.84 mmol, 1 eq) in ACN (32 mL) was added TMSI (1.78 g, 8.90 mmol, 1.3 eq) at 0° C. The mixture was stirred at 0° C. for 0.5 h. On completion, the reaction mixture was quenched by addition sat. NaHCO₃ (30 mL) at 0° C. to adjust pH=8, the mixture was diluted with water (50 mL) and extracted with ethyl acetate (50 mL×3), the combined organic phase was dried over anhydrous sodium sulfate, filtered and concentrated to give a residue. The residue was purified by column chromatography (SiO₂, DCM/MeOH=1:1 to 1:1) to give 2-[2-methyl-4-(1-tetrahydropyran-2-yl-3-vinyl-indazol-5-yl)pyrazol-3-yl]oxyethanamine (1.90 g, 5.12 mmol, 75% yield, 99% purity) as a colorless liquid. LCMS: m/z 368.0 (M+1).

Step 3. To a solution of 2-[2-methyl-4-(1-tetrahydropyran-2-yl-3-vinyl-indazol-5-yl)pyrazol-3-yl]oxyethanamine (800 mg, 2.18 mmol, 1 eq) and 5-ethyl-4-iodo-1-methyl-pyrazole-3-carbaldehyde (344 mg, 1.31 mmol, 0.6 eq) in MeOH (10 mL) was added KOAc (1.07 g, 10.8 mmol, 5 eq). The mixture was stirred at 25° C. for 0.5 h. Then NaBH₃CN (273 mg, 4.35 mmol, 2 eq) was added. The mixture was stirred at 25° C. for 1 h. On completion, the mixture was diluted with water (20 mL) and extracted with ethyl acetate (25 mL×3), the combined organic phase was dried over anhydrous sodium sulfate, filtered and concentrated to give a residue. The residue was purified by column chromatography (SiO₂, PE:THF=1:0 to 1:2) to give N-[(5-ethyl-4-iodo-1-methyl-pyrazol-3-yl)methyl]-2-[2-methyl-4-(1-tetrahydropyran-2-yl-3-vinyl-indazol-5-yl)pyrazol-3-yl]oxy-ethanamine (700 mg, 0.841 mmol, 39% yield, 74% purity) as a white solid. ¹H NMR (400 MHz, MeOD-d₄) δ=8.06 (s, 1H), 7.72-7.63 (m, 3H), 7.06-6.97 (m, 1H), 6.17-6.09 (m, 1H), 5.82-5.73 (m, 1H), 5.55-5.48 (m, 2H), 4.10-4.03 (m, 2H), 4.02-3.98 (m, 1H), 3.82 (s, 3H), 3.77 (s, 3H), 3.66 (s, 2H), 2.91 (t, J=5.6 Hz, 2H), 2.70 (q, J=7.6 Hz, 2H), 2.55-2.43 (m, 1H), 2.22 (s, 4H), 1.90-1.74 (m, 2H), 1.16-1.09 (m, 3H); LCMS: m/z 615.9 (M+1).

Step 4. To a solution of N-[(5-ethyl-4-iodo-1-methyl-pyrazol-3-yl)methyl]-2-[2-methyl-4-(1-tetrahydropyran-2-yl-3-vinyl-indazol-5-yl)pyrazol-3-yl]oxy-ethanamine (650 mg, 1.06 mmol, 1 eq) in DCM (6.5 mL) was added Ac₂O (162 mg, 1.58 mmol, 1.5 eq) and TEA (321 mg, 3.17 mmol, 3 eq). The mixture was stirred at 25° C. for 2 h. On completion, the mixture was diluted with water (15 mL) and extracted with DCM (25 mL×3), the combined organic phase was dried over anhydrous sodium sulfate, filtered and concentrated to give a residue. The residue was purified by column chromatography (SiO₂, DCM/MeOH=10:1 to 1:1) to give N-[(5-ethyl-4-iodo-1-methyl-pyrazol-3-yl)methyl]-N-[2-[2-methyl-4-(1-tetrahydropyran-2-yl-3-vinyl-indazol-5-yl)pyrazol-3-yl]oxyethyl]acetamide (360 mg, 0.515 mmol, 49% yield, 94% purity) as a colorless oil.

Steps 5 and 6 were performed in a similar manner to steps 3 and 4 in General Method T.

### Preparation of 2-[(10S,17E)-16-ethoxy-6-ethynyl-8,10,12-trimethyl-2,8,10,11,12,13-hexahydro-14H-3,5-ethenotripyrazolo|3,4-f:3′,4′-j:4″,3″-n][1,4]oxazacyclopentadecin-14-yl]ethan-1-ol (Ex. 292)

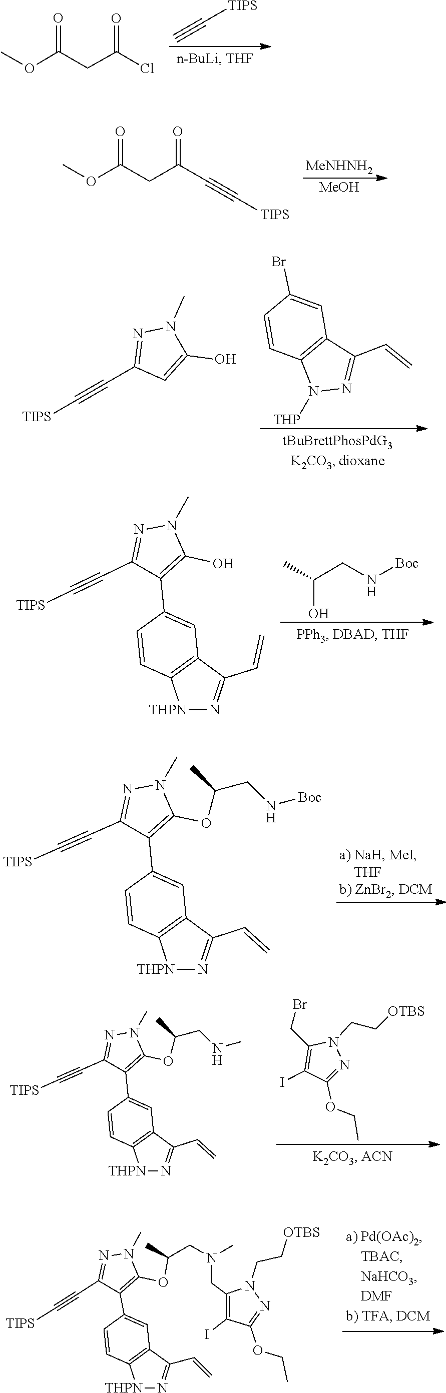

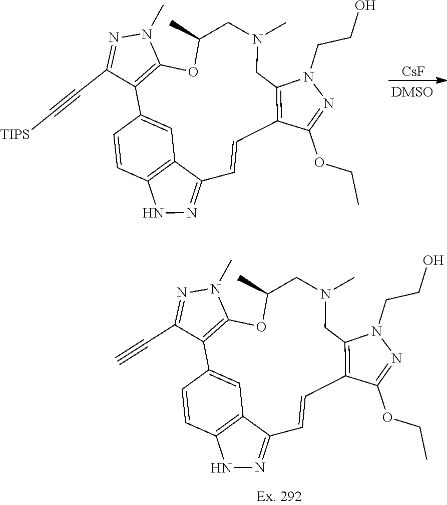

Step 1. To a solution of ethynyl(triisopropyl)silane (33.4 g, 183 mmol, 41 mL, 2.5 eq) in THF (200 mL) at −78° C. was added n-BuLi (2.5 M, 88 mL, 3 eq) under N₂. The mixture was stirred at the −78° C. for 1 hr, and then BF₃·Et₂O (26.0 g, 183 mmol, 23 mL, 2.5 eq) was added. After 0.2 hr, methyl 3-chloro-3-oxo-propanoate (10.0 g, 73.2 mmol, 7.82 mL, 1 eq) in THF (50 mL) was added. The mixture was stirred at −78° C. for 2 h. On completion, the mixture was quenched with sat. NH₄Cl (500 mL) and water (500 mL) and extracted with ethyl acetate (500 mL×3), the combined organic phase was dried over anhydrous sodium sulfate, filtered and concentrated to give methyl 3-oxo-5-triisopropylsilyl-pent-4-ynoate (60.0 g, crude) as an orange oil. The residue was purified by column chromatography (SiO₂, Petroleum ether/Ethyl acetate=1/0 to 10/1) to give methyl 3-oxo-5-triisopropylsilyl-pent-4-ynoate (9.96 g, 35.3 mmol, 17% yield) as a red oil. ¹H NMR (400 MHz, CDCl₃-d) δ=11.78-11.58 (m, 1H), 3.78-3.74 (m, 4H), 3.62-3.60 (m, 1H), 1.11-1.10 (m, 21H).

Step 2. To a solution of methyl 3-oxo-5-triisopropylsilyl-pent-4-ynoate (6.83 g, 24.2 mmol, 1 eq) in MeOH (70 mL) was added methylhydrazine (3.34 g, 29.0 mmol, 4 mL, 1.2 eq) at 0° C. The mixture was stirred at 25° C. for 16 hr. On completion, the mixture was filtered and concentrated to give a residue. The residue was purified by column chromatography (SiO₂, DCM:MeOH=1:0 to 10:1) to give 2-methyl-5-(2-triisopropylsilylethynyl)pyrazol-3-ol (3.15 g, 10.3 mmol, 43% yield, 91% purity) as a brown solid. ¹H NMR (400 MHz, DMSO-d6) δ=11.46-11.02 (m, 1H), 5.48 (s, 1H), 3.50 (s, 3H), 1.07 (s, 21H); LCMS: m/z 279.2 (M+1).

Step 3. To a solution of 2-methyl-5-(2-triisopropylsilylethynyl)pyrazol-3-ol (1.00 g, 3.59 mmol, 1 eq) in dioxane (10 mL) was added 5-bromo-1-tetrahydropyran-2-yl-3-vinyl-indazole (1.21 g, 3.95 mmol, 1.1 eq) and [2-(2-aminophenyl)phenyl]-methylsulfonyloxy-palladium;ditert-butyl-[3,6-dimethoxy-2-(2,4,6-triisopropylphenyl)phenyl]phosphane (307 mg, 0.359 mmol, 0.1 eq) then the mixture was added K₂CO₃ (993 mg, 7.18 mmol, 2 eq) under N₂. The mixture was stirred at 100° C. for 3 hr. On completion, the mixture was filtered and concentrated to give a residue. The residue was purified by column chromatography (SiO₂, DCM/MeOH=1/0 to 10/1) to give 2-methyl-4-(1-tetrahydropyran-2-yl-3-vinyl-indazol-5-yl)-5-(2-triisopropylsilylethynyl)pyrazol-3-ol (960 mg, 1.77 mmol, 49% yield, 93% purity) as a brown solid. ¹H NMR (400 MHz, DMSO-d₆) δ=11.60-10.26 (m, 1H), 8.15 (s, 1H), 7.75 (s, 1H), 7.69 (s, 1H), 6.97 (dd, J=11.4, 18.0 Hz, 1H), 6.05 (d, J=18.0 Hz, 1H), 5.84 (dd, J=2.4, 9.6 Hz, 1H), 5.50 (d, J=12.4 Hz, 1H), 3.88 (br d, J=11.2 Hz, 1H), 3.79-3.71 (m, 1H), 3.63 (s, 3H), 2.45-2.31 (m, 2H), 2.08-2.00 (m, 1H), 1.96 (br dd, J=2.4, 13.1 Hz, 1H), 1.76 (td, J=3.2, 6.3 Hz, 2H), 1.59 (br d, J=3.6 Hz, 3H), 1.05-1.01 (m, 18H); LCMS: m/z 505.2 (M+1).

Steps 4 through 9 were performed in a similar manner General Method Y to give 2-|(8S,17E)-15-ethoxy-5,8,10-trimethyl-3-(2-triisopropylsilylethynyl)-7-oxa-4,5,10,13,14,20,21-heptazapentacyclo[17.5.2.0^{2,6}.0^{12,16}0.0^{22,26}]hexacosa-1(25),2(6),3,12(16),14,17,19,22(26),23-nonaen-13-yl]ethanol (300 mg, crude) as a brown oil. LCMS: m/z 658.3 (M+1).

Step 10. To a solution of 2-[(8S,17E)-15-ethoxy-5,8,10-trimethyl-3-(2-triisopropylsilylethynyl)-7-oxa-4,5,10,13,14,20,21-heptazapentacyclo[17.5.2.0^{2,6}0.^{12,16}.0^{22,26}]hexacosa-1(25),2(6),3,12(16),14,17,19,22(26),23-nonaen-13-yl]ethanol (300 mg, 0.456 mmol, 1 eq) in DMSO (5 mL) was added CsF (416 mg, 2.74 mmol, 100 L, 6 eq). The mixture was stirred at 25° C. for 5 hr. On completion, the mixture was filtered and concentrated to give a residue. The residue was purified by prep-HPLC (FA condition; column: Phenomenex luna C18 150*25 mm*10 um; mobile phase: [water(FA)-ACN];gradient:11%-41% B over 15 min) to give 2-[(10S,17E)-16-ethoxy-6-ethynyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (30.43 mg, 0.061 mmol, 13% yield, 100% purity) as a white solid.

Example 293 was prepared using similar procedures to Example 285.

Example 294 was prepared using similar procedures to Example 291.

### Preparation of 2-[(10S,17E)-16-(ethylamino)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 295)

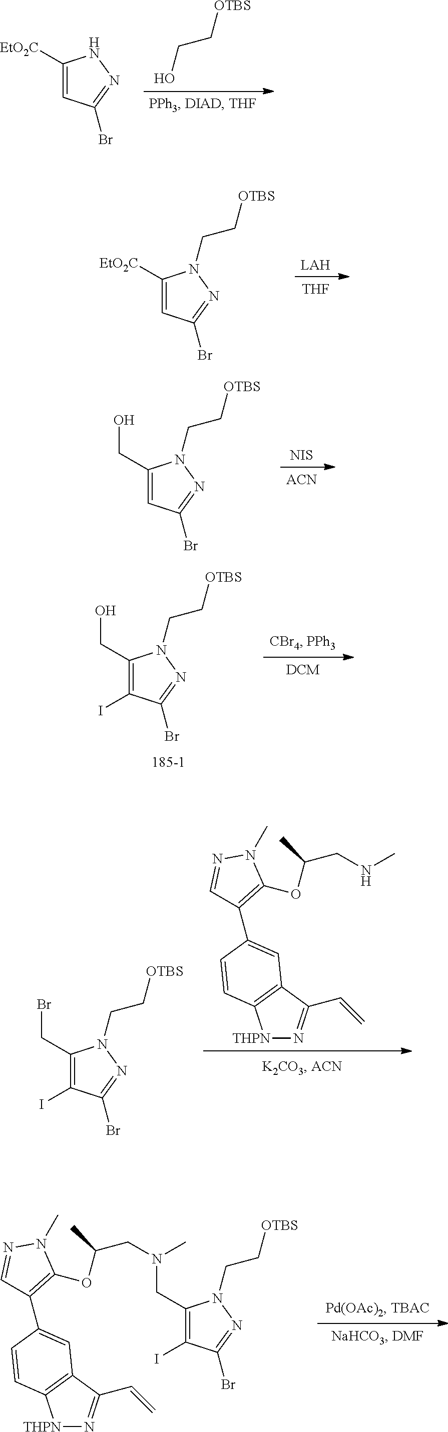

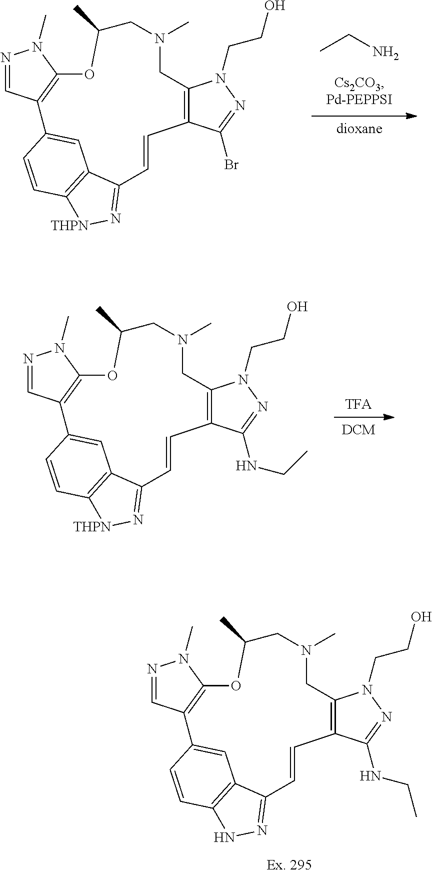

Steps 4 was conducted similarly to General Method 0 steps 5.

Steps 5-6 were conducted similarly to General Method Y steps 4-5.

Step 7. A mixture of 2-[(8S,17E)-15-bromo-5,8,10-trimethyl-21-tetrahydropyran-2-yl-7-oxa-4,5,10,13,14,20,21-heptazapentacyclo[17.5.2.0^{2,6}.0^{12,16}0.0^{22,26}]hexacosa-1(25),2(6), 3,12(16),14,17,19,22(26),23-nonaen-13-yl]ethanol (200 mg, 0.017 mmol, 1 eq), ethanamine (2 M, 17.4 μL, 2 eq), Cs₂CO₃ (17.0 mg, 0.052 mmol, 3 eq), 1,3-bis[2,6-bis(1-propylbutyl)phenyl]-4,5-dichloro-2H-imidazol-1-ium-2-ide;3-chloropyridine; dichloropalladium (1.70 mg, 0.002 mmol, 0.1 eq) in dioxane (2 mL) was degassed and purged with N₂ for 3 times, and then the mixture was stirred at 100° C. for 16 h under N₂ atmosphere. On completion, the reaction mixture was filtered and concentrated under reduced pressure to give a residue. The residue was purified by column chromatography (SiO₂, Petroleum ether/Ethyl acetate=1/0 to 0/1) to give 2-[(8S,17E)-15-(ethylamino)-5,8,10-trimethyl-21-tetrahydropyran-2-yl-7-oxa-4,5,10,13,14,20,21-heptazapentacyclo[17.5.2.0^{2,6}.0^{12,16}.0^{22,26}]hexacosa-1(25),2(6),3, 12(16),14,17,19, 22(26),23-nonaen-13-yl]ethanol (74.0 mg, crude) as a yellow solid. LCMS: m/z 561.1 (M+1).

Step 8 was performed in a similar manner to step 4 in General Method Y.

Example 296 was prepared using similar procedures to Example 280 using I-251 instead of I-252. Briefly, the synthesis of Example 296 was carried out as follows:

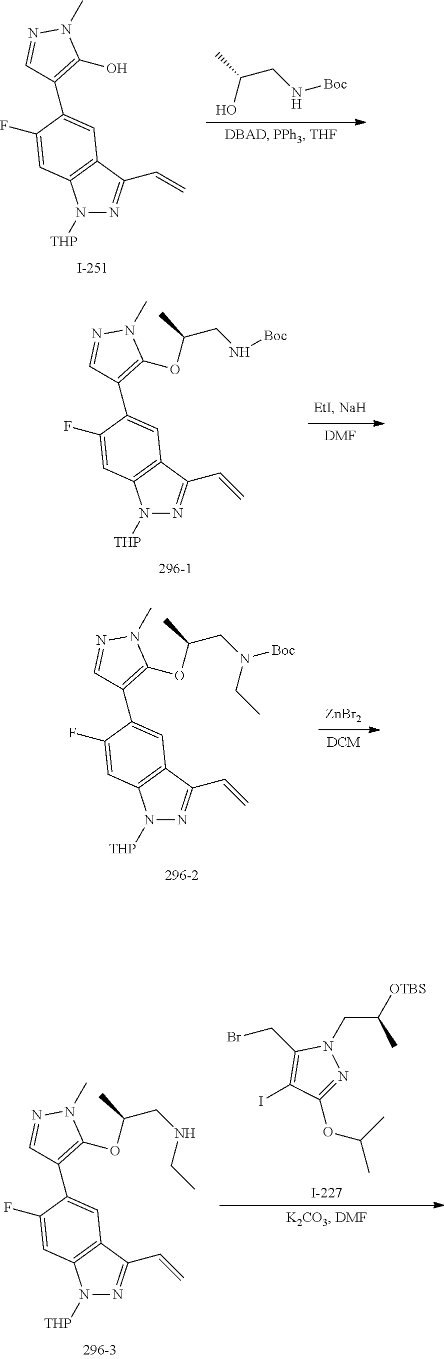

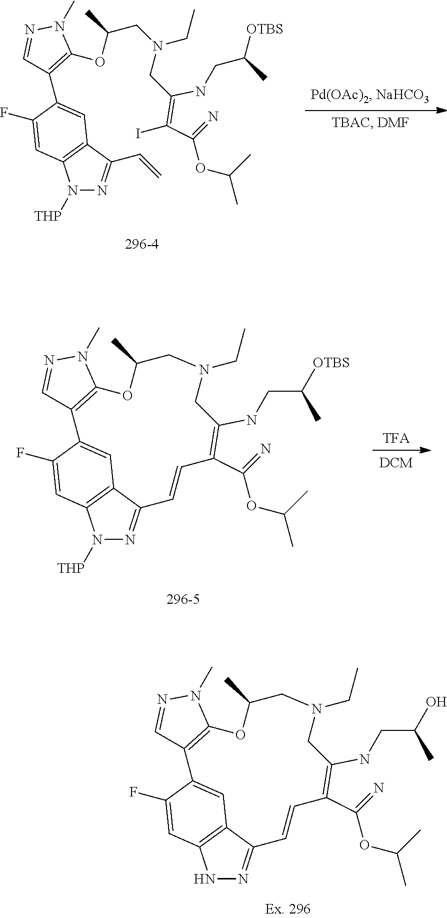

### Preparation of (2S)-1-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol (Ex. 296)

Step 1: A mixture of 4-(6-fluoro-1-tetrahydropyran-2-yl-3-vinyl-indazol-5-yl)-2-methyl-pyrazol-3-ol (I-251; 8.90 g, 26.0 mmol, 1 eq), PPh₃ (15.0 g, 57.2 mmol, 2.2 eq), DBAD (13.2 g, 57.2 mmol, 2.2 eq) was degassed and purged with N₂ for 3 times, THF (90 mL) and tert-butyl N-[(2R)-2-hydroxypropyl]carbamate (9.11 g, 52.0 mmol, 2 eq) was added, then the mixture was stirred at 25° C. for 2 hr under N₂ atmosphere. On completion, the reaction mixture was concentrated under reduced pressure to remove solvent. The residue was purified by column chromatography (SiO₂, Petroleum ether/Ethyl acetate=1/0 to 0/1) to give tert-butyl N-[(2S)-2-[4-(6-fluoro-1-tetrahydropyran-2-yl-3-vinyl-indazol-5-yl)-2-methyl-pyrazol-3-yl]oxypropyl]carbamate (296-1; 22.0 g, 22.0 mmol, 85% yield, 50% purity) as red oil. LCMS: (M+1)=500.2.

Step 2: To a solution of tert-butyl N-[(2S)-2-[4-(6-fluoro-t-tetrahydropyran-2-yl-3-vinyl-indazol-5-yl)-2-methyl-pyrazol-3-yl]oxypropyl]carbamate (296-1; 11.0 g, 22.0 mmol, 1 eq) in DMF (110 mL) was added NaH (1.76 g, 44.0 mmol, 60% purity, 2 eq) at 0° C. and stirred at 25° C. for 30 min. EtI (5.15 g, 33.0 mmol, 2.64 mL, 1.5 eq) was added at 0° C. The mixture was stirred at 25° C. for 2 hr. On completion, the reaction mixture was quenched by addition NH₄Cl (40 mL) at 0° C., and then diluted with H₂O (20 mL) and extracted with EA (30 mL*3). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give tert-butyl N-ethyl-N-[(2S)-2-[4-(6-fluoro-1-tetrahydropyran-2-yl-3-vinyl-indazol-5-yl)-2-methyl-pyrazol-3-yl]oxypropyl]carbamate (296-2; 8.58 g, 16.3 mmol, 74% yield) as red oil.

LCMS: (M+1)=528.2.

Step 3: To a solution of tert-butyl N-ethyl-N-[(2S)-2-[4-(6-fluoro-1-tetrahydropyran-2-yl-3-vinyl-indazol-5-yl)-2-methyl-pyrazol-3-yl]oxypropyl]carbamate (296-2; 7.50 g, 14.2 mmol, 1 eq) in DCM (75 mL) was added ZnBr₂ (9.60 g, 42.6 mmol, 3 eq). The mixture was stirred at 25° C. for 16 hr. On completion, the reaction mixture was concentrated under reduced pressure to remove solvent to give (2S)—N-ethyl-2-[4-(6-fluoro-1-tetrahydropyran-2-yl-3-vinyl-indazol-5-yl)-2-methyl-pyrazol-3-yl]oxy-propan-1-amine (296-3; 18.7 g, 13.1 mmol, 92% yield, 30% purity) as yellow solid.

LCMS: (M+1)=428.1.

Step 4: To a solution of (2S)—N-ethyl-2-[4-(6-fluoro-1-tetrahydropyran-2-yl-3-vinyl-indazol-5-yl)-2-methyl-pyrazol-3-yl]oxy-propan-1-amine (296-3; 1.93 g, 4.51 mmol, 1 eq,), [(1S)-2-[5-(bromomethyl)-4-iodo-3-isopropoxy-pyrazol-1-yl]-1-methyl-ethoxy]-tert-butyl-dimethyl-silane (I-227; 1.40 g, 2.71 mmol, 0.6 eq) in DMF (20 mL) was added K₂CO₃ (1.87 g, 13.5 mmol, 3 eq).The mixture was stirred at 60° C. for 16 h. On completion, the mixture was quenched by water (60 mL) at 0° C. and extracted with ethyl acetate (30 mL×3). The combined organic phase was washed with water (100 mL*2) and dried over Na₂SO₄, filtered and the filtrate was concentrated to give a residue. The residue was purified by column chromatography (SiO₂, PE:THF=4:1) to give (2S)—N-[[2-[(2S)-2-[tert-butyl(dimethyl)silyl]oxypropyl]-4-iodo-5-isopropoxy-pyrazol-3-yl]methyl]-N-ethyl-2-[4-(6-fluoro-1-tetrahydropyran-2-yl-3-vinyl-indazol-5-yl)-2-methyl-pyrazol-3-yl]oxy-propan-1-amine (296-4; 850 mg, crude) as a yellow oil.

LCMS: (M+1)=864.2

Step 5: To a solution of (2S)—N-[[2-[(2S)-2-[tert-butyl(dimethyl)silyl]oxypropyl]-4-iodo-5-isopropoxy-pyrazol-3-yl]methyl]-N-ethyl-2-[4-(6-fluoro-1-tetrahydropyran-2-yl-3-vinyl-indazol-5-yl)-2-methyl-pyrazol-3-yl]oxy-propan-t-amine (296-4; 750 mg, 868 mol, 1 eq) in DMF (37 mL) was added NaHCO₃ (182 mg, 2.17 mmol, 2.5 eq), Pd(OAc)₂ (39.0 mg, 174 μmol, 0.2 eq) and TBAC (241 mg, 868 mol, 1 eq).

The mixture was stirred at 130° C. for 1 h. On completion, the mixture was quenched by water (100 mL) and extracted with ethyl acetate (40 mL×3). The combined organic phase was washed with water (100 mL*2) and dried over Na₂SO₄, filtered and the filtrate was concentrated to give a residue. The residue was purified by column chromatography (SiO₂, PE:EA=2:1) to give tert-butyl-[(1S)-2-[(8S,17E)-10-ethyl-24-fluoro-15-isopropoxy-5,8-dimethyl-21-tetrahydropyran-2-yl-7-oxa-4,5,10,13,14,20,21-heptazapentacyclo[17.5.2.0^{2,6}. 0^{12,16}.0^{22,26}]hexacosa-1(25),2(6),3,12(16),14,17,19,22(26),23-nonaen-13-yl]-1-methyl-ethoxyl-dimethyl-silane (296-5; 445 mg, crude) as a yellow solid.

LCMS: (M+1)=736.3.

Step 6: To a solution of tert-butyl-[(1S)-2-[(8S,17E)-10-ethyl-24-fluoro-15-isopropoxy-5,8-dimethyl-21-tetrahydropyran-2-yl-7-oxa-4,5,10,13,14,20,21-heptazapentacyclo [17.5.2.0^{2,6}.0^{12,16}0.0^{22,26}]hexacosa-1(25),2(6),3,12(16),14,17,19,22(26),23-nonaen-13-yl]-1-methyl-ethoxy]-dimethyl-silane (296-5; 200 mg, 272 mol, 1 eq) in DCM (2 mL) was added TFA (9.42 mmol, 0.7 mL, 34.7 eq). The mixture was stirred at 25° C. for 16 h. On completion, the mixture was concentrated to give a residue. The residue was purified by prep-HPLC (FA condition; column: Phenomenex luna C18 150*25 mm* 10 um; mobile phase: [water(FA)-ACN];gradient:23%-43% B over 10 min) to give (2S)-1-[(8S,17E)-10-ethyl-24-fluoro-15-isopropoxy-5,8-dimethyl-7-oxa-4,5,10,13,14,20,21-heptazapentacyclo[17.5.2.0^{2,6}.0^{12,16}0.0^{22,26}]hexacosa-1(25),2(6),3,12(16),14,17,19,22 (26),23-nonaen-13-yl]propan-2-ol (ex. 296; 46.51 mg, 86.51 mol, 31.84% yield) as an off-white solid.

¹H NMR (400 MHz, MeOH-d₄) δ=8.65 (br d, J=7.2 Hz, 1H), 8.06 (d, J=16.8 Hz, 1H), 7.80 (d, J=4.8 Hz, 1H), 7.29-7.19 (m, 2H), 5.03-4.92 (m, 1H), 4.75-4.65 (m, 1H), 4.14-4.00 (m, 2H), 3.99-3.91 (m, 3H), 3.75 (s, 3H), 3.07-2.85 (m, 2H), 2.69-2.51 (m, 2H), 1.48-1.42 (m, 6H), 1.21 (d, J=6.0 Hz, 3H), 1.08 (d, J=6.4 Hz, 3H), 0.96 (t, J=7.2 Hz, 3H).

LCMS: (M+1)=538.1.

### Example 297

### Preparation of 2-[(10S,17E)-6-amino-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol (Ex. 297)

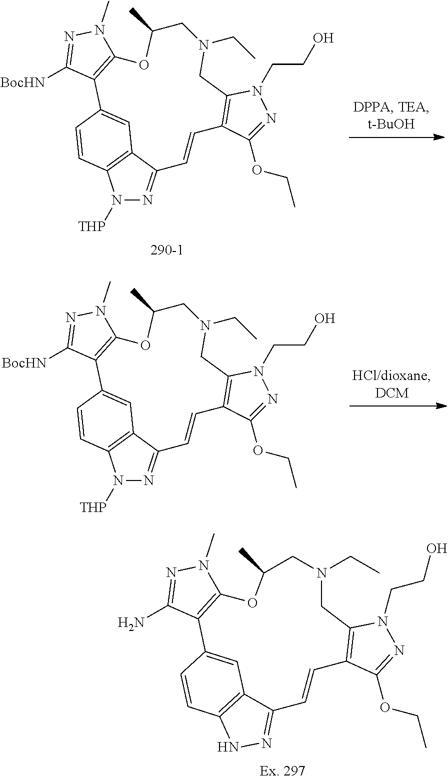

Step 1. A mixture of (8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaene-3-carboxylic acid (120 mg, 0.193 mmol, 1 eq), DPPA (106 mg, 0.387 mmol, 2 eq), TEA (39.1 mg, 0.387 mmol, 2 eq) in t-BuOH (2 mL) was degassed and purged with N₂ for 3 times, and then the mixture was stirred at 90° C. for 0.5 hours under N₂ atmosphere. On completion, the mixture was filtered and concentrated. The residue was purified by column chromatography (SiO₂, Petroleum ether/THF=1:0 to 0:1) to give tert-butyl N-[(8S,17E)-15-ethoxy-10-ethyl-13-(2-hydroxyethyl)-5,8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaen-3-yl]carbamate (52.0 mg, 0.075 mmol, 38% yield) as a yellow solid. LCMS: m/z 691.3 (M+1).

Step 2. To a solution of tert-butyl N-[(8S,17E)-15-ethoxy-10-ethyl-13-(2-hydroxyethyl)-5, 8-dimethyl-21-tetrahydropyran-2-yl-7-oxa-4,5,10,13,14,20,21-heptazapentacyclo [17.5.2.02,6.012,16.022,26]hexacosa-1(25),2(6),3,12(16),14,17,19,22(26),23-nonaen-3-yl]carbamate (20.0 mg, 0.029 mmol, 1 eq) in DCM (0.5 mL) was added HCl/dioxane (4 M, 0.1 mL, 10 eq). The mixture was stirred at 25° C. for 0.1 hour. On completion, the mixture was concentrated to give a residue. The residue was purified by prep-HPLC (column: Welch Ultimate C18 150*25 mm*5 um; mobile phase: [water(FA)-ACN]; gradient:5%-35% B over 10 min) to give 2-[(8S,17E)-3-amino-15-ethoxy-10-ethyl-5,8-dimethyl-7-oxa-4,5,10,13,14,20,21-heptazapentacyclo [17.5.2.02,6.012,16.022,26]hexacosa-1(25),2(6),3,12(16),14,17,19,22(26),23-nonaen-13-yl]ethanol (9.08 mg, 0.016 mmol, 57% yield, 100% purity, FA) as a brown solid.

Example 298 was prepared using similar procedures to Example 289.

### Example 299

### Preparation of 2,2′-[(10S,17E)-10,12-dimethyl-16-[(propan-2-yl)oxy]-10,11,12,13-tetrahydro-8H-3,5-ethenotripyrazolo[3,4-f:3′,4′-j:4″,3″-n][1,4]oxazacyclopentadecine-8,14(2H)-diyl]di(ethan-1-ol) (Ex. 299)

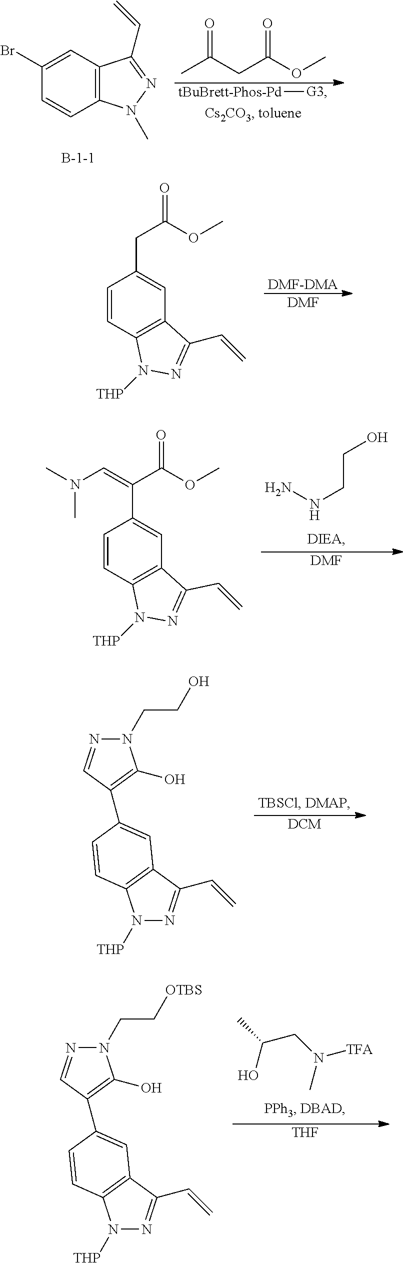

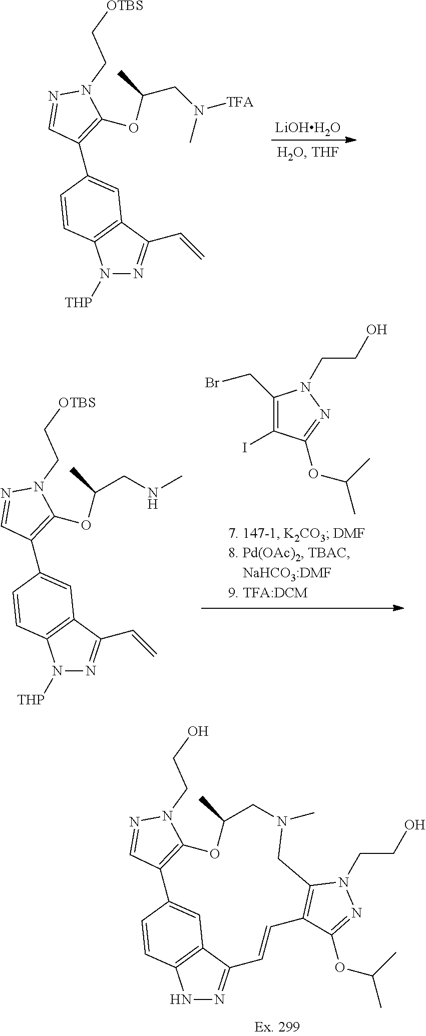

Step 1. A solution of 5-bromo-1-tetrahydropyran-2-yl-3-vinyl-indazole (5.00 g, 16.3 mmol, 1 eq), methyl 3-oxobutanoate (2.83 g, 24.4 mmol, 1.5 eq), tBuBrett-Phos-Pd-G3 (695 mg, 0.814 mmol, 0.05 eq), Cs₂CO₃ (15.9 g, 48.8 mmol, 3 eq) in toluene (60 mL) was degassed and purged with N₂ for 3 times, and then the mixture was stirred at 70° C. for 12 hours under N₂ atmosphere. On completion, the mixture was filtered and concentrated to give a residue. The residue was purified by column chromatography (SiO₂, PE:THF=3:1 to 3:1) to give methyl 2-(1-tetrahydropyran-2-yl-3-vinyl-indazol-5-yl) acetate (3.30 g, 11.0 mmol, 67% yield) as a yellow oil. LCMS: m/z 301.0 (M+1)

Step 2. To a solution of methyl 2-(1-tetrahydropyran-2-yl-3-vinyl-indazol-5-yl) acetate (3.00 g, 9.99 mmol, 1 eq) in DMF (20 mL) was added DMF-DMA (2.38 g, 19.9 mmol, 2 eq). The mixture was stirred at 60° C. for 12 h. On completion, the mixture was concentrated to give methyl (E)-3-(dimethylamino)-2-(1-tetrahydropyran-2-yl-3-vinyl-indazol-5-yl) prop-2-enoate (3.80 g, crude) as a yellow oil. LCMS: m/z 329.0 (M+1-27)

Step 3. To a solution of methyl (E)-3-(dimethylamino)-2-(1-tetrahydropyran-2-yl-3-vinyl-indazol-5-yl)prop-2-enoate (3.80 g, 10.7 mmol, 1 eq) in DMF (38 mL) was added DIEA (4.15 g, 32.1 mmol, 3 eq) and 2-hydrazinoethanol (1.22 g, 16.0 mmol, 1.5 eq). The mixture was stirred at 60° C. for 12 h. On completion, the mixture was concentrated to give a residue. The residue was purified by column chromatography (SiO₂, DCM:MeOH=10:1 to 10:1) to give 2-(2-hydroxyethyl)-4-(1-tetrahydropyran-2-yl-3-vinyl-indazol-5-yl)pyrazol-3-ol (2.00 g, 5.64 mmol, 53% yield) as a brown solid. LCMS: m/z 355.0 (M+1)

Step 4. To a solution of 2-(2-hydroxyethyl)-4-(1-tetrahydropyran-2-yl-3-vinyl-indazol-5-yl)pyrazol-3-ol (500 mg, 1.41 mmol, 1 eq) in DCM (5 mL) was added DMAP (689 mg, 5.64 mmol, 4 eq) and TBSCl (638 mg, 4.23 mmol 3 eq) at 0° C. The mixture was stirred at 25° C. for 1 h. On completion, the reaction mixture was partitioned between DCM (5 mL×3) and water (5 mL), the combined organic phase was dried over anhydrous sodium sulfate, filtered and concentrated to give a residue. The residue was purified by column chromatography (SiO₂, DCM:MeOH=10:1 to 10:1) to give 2-[2-[tert-butyl(dimethyl)silyl]oxyethyl]-4-(1-tetrahydropyran-2-yl-3-vinyl-indazol-5-yl)pyrazol-3-ol (600 mg, 1.28 mmol, 91% yield) as a brown solid. LCMS: m/z 469.1 (M+1)

Step 5. To a solution of 2-[2-[tert-butyl(dimethyl)silyl]oxyethyl]-4-(1-tetrahydropyran-2-yl-3-vinyl-indazol-5-yl)pyrazol-3-ol (510 mg, 1.09 mmol, 1 eq) in THF (5 mL) was added 2,2,2-trifluoro-N-[(2R)-2-hydroxypropyl]-N-methyl-acetamide (604 mg, 3.26 mmol, 3 eq), PPh₃ (571 mg, 2.18 mmol, 2 eq), and DBAD (376 mg, 1.63 mmol, 1.5 eq) under N₂. The mixture was stirred at 25° C. for 2 h. On completion, the mixture was concentrated to give a residue. The residue was purified by column chromatography (SiO₂, PE:THF=4:1 to 4:1) to give N-[(2S)-2-[2-[2-[tert-butyl(dimethyl) silyl]oxyethyl]-4-(1-tetrahydropyran-2-yl-3-vinyl-indazol-5-yl)pyrazol-3-yl]oxypropyl]-2,2,2-trifluoro-N-methyl-acetamide (530 mg, 0.325 mmol, 30% yield, 39% purity) as a brown oil. LCMS: m/z 636.3 (M+1)

Step 6. To a solution of N-[(2S)-2-[2-[2-[tert-butyl(dimethyl)silyl]oxyethyl]-4-(1-tetrahydropyran-2-yl-3-vinyl-indazol-5-yl)pyrazol-3-yl]oxypropyl]-2,2,2-trifluoro-N-methyl-acetamide (480 mg, 0.754 mmol, 1 eq) in THF (5 mL) was added LiOH·H₂O (126 mg, 3.02 mmol, 4 eq) and H₂O (1 mL). The mixture was stirred at 25° C. for 16 h. On completion, the reaction mixture was partitioned between EA (7 mL×3) and water (7 mL), the combined organic phase was dried over anhydrous sodium sulfate, filtered and concentrated to give a residue. The residue was purified by column chromatography (SiO₂, DCM:MOH=10:1 to 10:1) to give (2S)-2-[2-[2-[tert-butyl(dimethyl)silyl]oxyethyl]-4-(1-tetrahydropyran-2-yl-3-vinyl-indazol-5-yl)pyrazol-3-yl]oxy-N-methyl-propan-1-amine (102 mg, 0.189 mmol, 25% yield) as a yellow oil. LCMS: m/z 540.0 (M+1)

The remaining steps were performed according to those described in General Method Y to afford Ex. 299 as a white solid.

| | | MS m/z | ¹H NMR (400 MHz, DMSO- |
|---|---|---|---|
| Ex # | Structure | [M + H]⁺ | d₆) δ ppm |
| 1 | 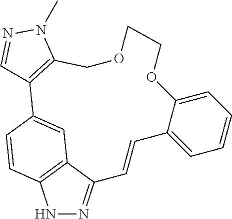 [SMILES: Cn1ncc2c1COCCOc1ccccc1/C=C/c1n[nH]c3ccc-2cc13] | 373.1 | 3.08 (s, 1H), 8.55 (s, 1H), 8.10 (d, J = 17.2 Hz, 1H), 7.91- 7.85 (m, 2H), 7.68 (d, J = 8.8 Hz, 1H), 7.54 (d, J = 8.8 Hz, 1H), 7.47 (d, J = 17.2 Hz, 1H), 7.34-7.27 (m, 1H), 7.26-7.20 (m, 1H), 7.09 (t, J = 7.6 Hz, 1H), 4.48 (t, J = 6.0 Hz, 2H), 4.28 (t, J = 5.2 Hz, 2H), 3.74 (s, 3H), 2.40 (td, J = 5.2, 10.8 Hz, 2H |
| 2 | 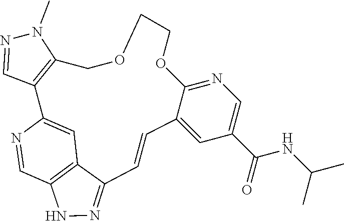 [SMILES: CC(C)NC(=O)c1cnc2c(c1)/C=C/c1n[nH]c3cnc(cc13)-c1cnn(C)c1COCCO2] | 459.1 | 13.23 (s, 1H), 8.66 (d, J = 2.0 Hz, 1H), 8.56 (d, J = 2.4 Hz, 1H), 8.30 (s, 1H), 8.26 (d, J = 7.6 Hz, 1H), 8.06 (d, J = 17.6 Hz, 1H), 7.69 (s, 1H), 7.66- 7.61 (m, 2H), 7.61-7.52 (m, 2H), 4.67 (s, 2H), 4.58-4.53 (m, 2H), 4.03-3.99 (m, 2H), 3.94 (s, 3H), 1.21 (d, J = 6.4 Hz, 6H) |
| 3 | 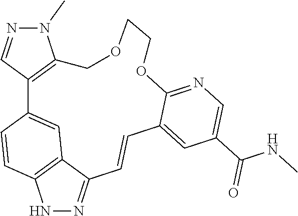 [SMILES: CNC(=O)c1cnc2c(c1)/C=C/c1n[nH]c3ccc(cc13)-c1cnn(C)c1COCCO2] | 431.2 | 13.24 (s, 1H), 8.63 (d, J = 1.6 Hz, 1H), 8.57-8.50 (m, 2H), 8.30 (s, 1H), 8.05 (d, J = 17.2 Hz, 1H), 7.69 (s, 1H), 7.66- 7.61 (m, 1H), 7.57 (d, J = 5.6 Hz, 1H), 7.54 (d, J = 2.8 Hz, 1H), 4.66 (s, 2H), 4.55 (d, J = 2.8 Hz, 2H), 4.01 (d, J = 2.0 Hz, 2H), 3.93 (s, 3H), 2.82 (d, J = 4.4 Hz, 3H) |
| 4 | 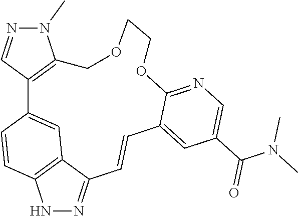 [SMILES: CN(C)C(=O)c1cnc2c(c1)/C=C/c1n[nH]c3ccc(cc13)-c1cnn(C)c1COCCO2] | 445.2 | 13.21 (s, 1H), 8.30 (s, 1H), 8.28 (d, J = 2.0 Hz, 1H), 8.16 (d, J = 2.0 Hz, 1H), 8.06 (d, J = 17.6 Hz, 1H), 7.69 (s, 1H), 7.64-7.58 (m, 2H), 7.56-7.53 (m, 1H), 4.66 (s, 2H), 4.56- 4.53 (m, 2H), 4.02-4.01 (m, 2H), 3.93 (s, 3H), 3.01 (s, 6H) |
| 5 | 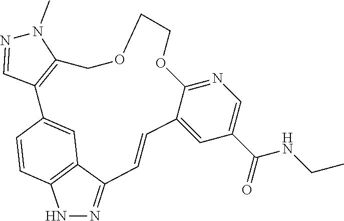 [SMILES: CCNC(=O)c1cnc2c(c1)/C=C/c1n[nH]c3ccc(cc13)-c1cnn(C)c1COCCO2] | 445.2 | 13.22 (s, 1H), 8.65 (d, J = 2.0 Hz, 1H), 8.56 (d, J = 2.0 Hz, 1H), 8.53-8.50 (m, 1H), 8.30 (s, 1H), 8.06 (d, J = 17.2 Hz, 1H), 7.69 (s, 1H), 7.65-7.59 (m, 2H), 7.57-7.54 (m, 1H), 4.67 (s, 2H), 4.58-4.55 (m, 2H), 4.02-4.00 (m, 2H), 3.93 (s, 3H), 3.31-3.28 (m, 2H), 1.17 (t, J = 7.6 Hz, 3H) |
| 6 | 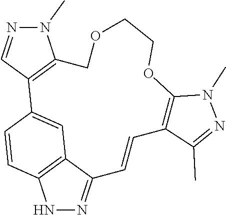 [SMILES: Cc1nn(C)c2c1/C=C/c1n[nH]c3ccc(cc13)-c1cnn(C)c1COCCO2] | 391.4 | 8.56-8.46 (m, 1H), 7.76-7.73 (m, 1H), 7.73-7.68 (m, 1H), 7.60-7.50 (m, 2H), 7.13-7.07 (m, 1H), 4.67-4.60 (m, 2H), 4.43 (d, J = 3.6 Hz, 2H), 4.03- 4.00 (m, 2H), 3.72 (s, 3H), 3.50 (s, 3H), 2.51 (s, 3H) |
| 7 | 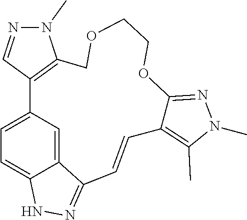 [SMILES: Cc1c2c(nn1C)OCCOCc1c(cnn1C)-c1ccc3[nH]nc(c3c1)/C=C/2] | 390.9 | 13.35-12.54 (m, 1H), 8.29 (s, 1H), 7.77 (s, 1H), 7.56 (s, 2H), 7.46 (d, J = 17.6 Hz, 1H), 6.98 (d, J = 17.6 Hz, 1H), 4.57 (s, 2H), 4.35-4.33 (m, 2H), 3.92 (s, 3H), 3.82-3.79 (m, 2H), 3.67 (s, 3H), 2.46 (s, 3H) |
| 8 | 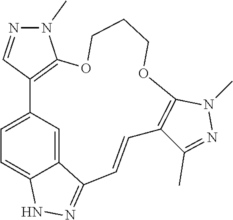 [SMILES: Cc1nn(C)c2c1/C=C/c1n[nH]c3ccc(cc13)-c1cnn(C)c1OCCCO2] | 390.9 | 13.06-12.78 (m, 1H), 8.56 (s, 1H), 7.91 (s, 1H), 7.71-7.66 (m, 1H), 7.49 (d, J = 8.8 Hz, 1H), 7.42 (d, J = 17.6 Hz, 1H), 6.91 (d, J = 17.2 Hz, 1H), 4.45 (t, J = 5.6 Hz, 2H), 4.34 (t, J = 4.8 Hz, 2H), 3.75 (s, 3H), 3.64 (s, 3H), 2.43-2.38 (m, 2H), 2.37 (s, 3H) |
| 9 | 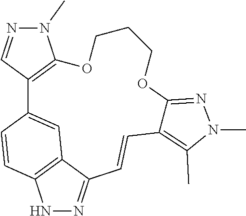 [SMILES: Cc1c2c(nn1C)OCCCOc1c(cnn1C)-c1ccc3[nH]nc(c3c1)/C=C/2] | 390.8 | 12.90 (s, 1H), 8.47 (s, 1H), 7.90 (s, 1H), 7.68 (dd, J = 1.6, 8.8 Hz, 1H), 7.49 (d, J = 8.8 Hz, 1H), 7.40 (d, J = 17.6 Hz, 1H), 6.93 (d, J = 17.6 Hz, 1H), 4.41 (t, J = 6.0 Hz, 2H), 4.28- 4.23 (m, 2H), 3.72 (s, 3H), 3.69 (s, 3H), 3.17 (s, 5H), 2.48 (s, 3H), 2.32-2.25 (m, 2H) |
| 10 | 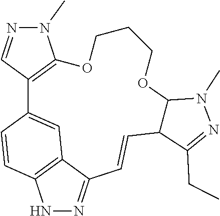 [SMILES: CCC1=NN(C)C2OCCCOc3c(cnn3C)-c3ccc4[nH]nc(c4c3)/C=C/C12] | 404.8 | 8.62 (s, 1H), 7.84 (s, 1H), 7.66 (dd, J = 1.2 , 8.8 Hz, 1H), 7.50 (d, J = 8.0 Hz, 1H), 7.46 (s, 1H), 6.94 (d, J = 17.0 Hz, 1H), 4.44 (t, J = 5.6 Hz, 2H), 4.36 (t, J = 5.0 Hz, 2H), 3.75 (s, 3H), 3.67 (s, 3H), 2.83 (q, J = 7.6 Hz, 2H), 2.42 (quin, J = 5.2 Hz, 2H), 1.33 (t, J = 7.6 Hz, 3H |
| 11 | 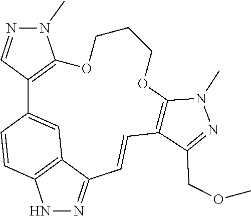 [SMILES: COCc1nn(C)c2c1/C=C/c1n[nH]c3ccc(cc13)-c1cnn(C)c1OCCCO2] | 421.1 | 8.56 (s, 1H), 7.91 (s, 1H), 7.68 (dd, J = 1.2, 8.8 Hz, 1H), 7.50 (d, J = 8.8 Hz, 1H), 7.38 (d, J = 16.0 Hz, 1H), 7.04 (d, J = 16.0 Hz, 1H), 4.47-4.44 (m, 4H), 4.38-4.35 (m, 2H), 3.74 (s, 3H), 3.70 (s, 3H), 3.32 (s, 3H), 2.44-2.37 (m, 2H) |
| 12 | 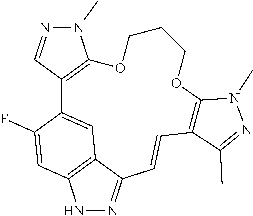 [SMILES: Cc1nn(C)c2c1/C=C/c1n[nH]c3cc(F)c(cc13)-c1cnn(C)c1OCCCO2] | 408.8 | 13.16-12.82 (m, 1H), 8.64 (d, J = 7.2 Hz, 1H), 7.78 (d, J = 5.2 Hz, 1H), 7.41-7.32 (m, 2H), 6.90 (d, J = 17.2 Hz, 1H), 4.49 (t, J = 5.6 Hz, 2H), 4.32 (t, J = 4.8 Hz, 1H), 4.38-4.26 (m, 1H), 3.78 (s, 3H), 3.64 (s, 3H), 2.39 (br d, J = 5.2 Hz, 2H), 2.36 (s, 3H), 2.32-2.31 (m, 1H) |
| 13 | 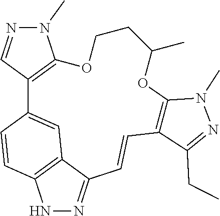 [SMILES: CCc1nn(C)c2c1/C=C/c1n[nH]c3ccc(cc13)-c1cnn(C)c1OCCC(C)O2] | 418.9 | (CDCl₃) δ = 8.67 (s, 1H), 7.85 (s, 1H), 7.73 (d, J = 9.2 Hz, 1H), 7.62 (d, J = 17.2 Hz, 1H), 7.57 (d, J = 8.8 Hz, 1H), 7.01 (d, J = 17.2 Hz, 1H), 5.01- 4.90 (m, 1H), 4.81-4.72 (m, 1H), 4.26-4.18 (m, 1H), 3.85 (s, 3H), 3.74 (s, 3H), 2.96- 2.89 (m, 2H), 2.56-2.31 (m, 2H), 1.44 (d, J = 6.0 Hz, 3H), 1.39 (t, J = 7.6 Hz, 3H) |
| 14 | 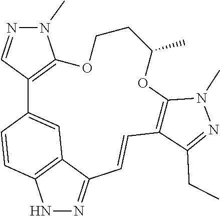 [SMILES: CCc1nn(C)c2c1/C=C/c1n[nH]c3ccc(cc13)-c1cnn(C)c1OCC[C@H](C)O2] | 419.0 | 12.92 (s, 1H), 8.55 (s, 1H), 7.90 (s, 1H), 7.66 (d, J = 8.8 Hz, 1H), 7.49 (d, J = 8.8 Hz, 1H), 7.41 (d, J = 17.2 Hz, 1H), 6.89 (d, J = 17.2 Hz, 1H), 4.81- 4.58 (m, 2H), 4.36-4.28 (m, 1H), 3.76 (s, 3H), 3.61 (s, 3H), 2.83-2.74 (m, 2H), 2.42-2.34 (m, 2H), 1.35 (d, J = 6.4 Hz, 3H), 1.27 (t, J = 7.2 Hz, 3H) |
| 15 | 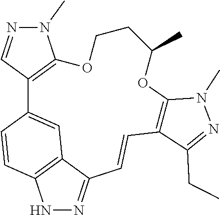 [SMILES: CCc1nn(C)c2c1/C=C/c1n[nH]c3ccc(cc13)-c1cnn(C)c1OCC[C@@H](C)O2] | 419.0 | 12.94 (s, 1H), 8.55 (s, 1H), 7.90 (s, 1H), 7.70-7.63 (m, 1H), 7.49 (d, J = 8.8 Hz, 1H), 7.41 (d, J = 17.2 Hz, 1H), 6.89 (d, J = 17.2 Hz, 1H), 4.77- 4.59 (m, 2H), 4.37-4.27 (m, 1H), 3.76 (s, 3H), 3.61 (s, 3H), 2.81-2.75 (m, 2H), 2.41-2.35 (m, 2H), 1.35 (d, J = 6.4 Hz, 3H), 1.27 (t, J = 7.2 Hz, 3H) |
| 16 | 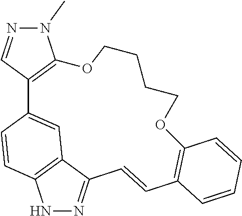 [SMILES: Cn1ncc2c1OCCCCOc1ccccc1/C=C/c1n[nH]c3ccc-2cc13] | 387.1 | 13.86-12.04 (m, 1H), 8.48 (s, 1H), 7.89-7.84 (m, 2H), 7.82- 7.79 (m, 1H), 7.71-7.68 (m, 1H), 7.56 (d, J = 4.0 Hz, 1H), 7.53 (d, J = 4.4 Hz, 1H), 7.30- 7.25 (m, 1H), 7.07 (d, J = 8.0 Hz, 1H), 7.00 (t, J = 7.2 Hz, 1H), 4.27 (t, J = 6.8 Hz, 2H), 4.16 (t, J = 5.2 Hz, 2H), 3.76 (s, 3H), 2.26-2.17 (m, 2H), 2.09-2.02 (m, 2H) |
| 17 | 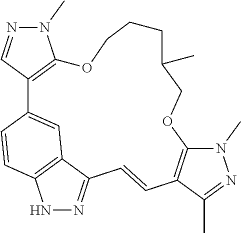 [SMILES: Cc1nn(C)c2c1/C=C/c1n[nH]c3ccc(cc13)-c1cnn(C)c1OCCCC(C)CO2] | 433.2 | 8.21-8.19 (m, 1H), 7.75-7.73 (m, 1H), 7.60 (dd, J = 1.2, 8.8 Hz, 1H), 7.52-7.48 (m, 1H), 7.30-7.18 (m, 2H), 4.09-3.99 (m, 3H), 3.85 (dd, J = 6.2, 8.4 Hz, 1H), 3.78 (s, 3H), 3.69 (s, 3H), 2.31 (s, 3H), 2.28-2.19 (m, 1H), 2.09-1.96 (m, 2H), 1.88-1.73 (m, 2H), 1.14 (d, J = 6.8 Hz, 3H) |
| 18 | 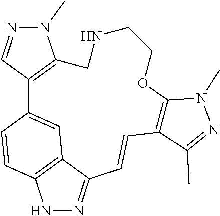 [SMILES: Cc1nn(C)c2c1/C=C/c1n[nH]c3ccc(cc13)-c1cnn(C)c1CNCCO2] | 390.0 | 13.21-12.83 (m, 1H), 9.83- 9.42 (m, 1H), 8.28-8.12 (m, 1H), 7.95-7.85 (m, 1H), 7.58 (s, 2H), 7.54 (d, J = 18.0 Hz, 1H), 6.96 (d, J = 17.2 Hz, 1H), 4.68-4.56 (m, 2H), 4.55-4.43 (m, 2H), 4.03 (s, 3H), 3.67 (s, 3H), 2.72 (s, 2H), 2.37 (s, 3H) |
| 19 | 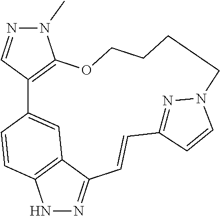 [SMILES: Cn1ncc2c1OCCCCn1ccc(n1)/C=C/c1n[nH]c3ccc-2cc13] | 360.8 | 13.34-12.51 (m, 1H), 8.08 (s, 1H), 7.84 (s, 1H), 7.77 (d, J = 2.0 Hz, 1H), 7.61 (d, J = 8.8 Hz, 1H), 7.47 (d, J = 8.8 Hz, 1H), 7.23 (s, 2H), 6.64 (d, J = 2.0 Hz, 1H), 4.21 (d, J = 6.0 Hz, 4H), 3.70 (s, 3H), 1.92- 1.78 (m, 4H) |
| 20 | 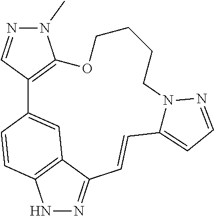 [SMILES: Cn1ncc2c1OCCCCn1nccc1/C=C/c1n[nH]c3ccc-2cc13] | 360.9 | 13.45-12.79 (m, 1H), 8.53 (s, 1H), 7.94 (s, 1H), 7.76-7.71 (m, 1H), 7.60-7.52 (m, 2H), 7.47-7.39 (m, 2H), 6.80 (d, J = 2.0 Hz, 1H), 4.44-4.34 (m, 4H), 3.73 (s, 3H), 2.02 (s, 4H) |
| 21 | 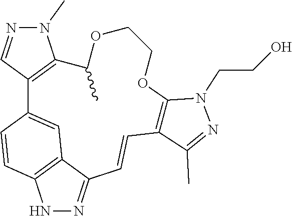 [SMILES: Cc1nn(CCO)c2c1/C=C/c1n[nH]c3ccc(cc13)-c1cnn(C)c1C(C)OCCO2] | 435.0 | 13.02-12.71 (m, 1H), 9.02- 8.80 (m, 1H), 7.64-7.58 (m, 2H), 7.49 (d, J = 1.8 Hz, 2H), 6.92-6.86 (m, 1H), 5.04-4.97 (m, 1H), 4.42-4.35 (m, 1H), 4.23 (t, J = 9.4 Hz, 1H), 4.06 (d, J = 9.4 Hz, 2H), 3.97 (s, 3H), 3.93 (d, J = 5.2 Hz, 3H), 3.71 (s, 2H), 2.39-2.35 (m, 3H), 1.32 (d, J = 6.4 Hz, 3H) |
| 22 | 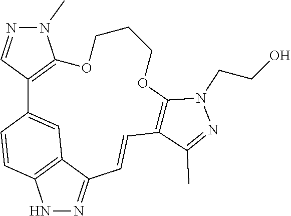 [SMILES: Cc1nn(CCO)c2c1/C=C/c1n[nH]c3ccc(cc13)-c1cnn(C)c1OCCCO2] | 421.1 | 8.58 (s, 1H), 7.92 (s, 1H), 7.69 (d, J = 8.8 Hz, 1H), 7.50 (d, J = 8.8 Hz, 1H), 7.33 (d, J = 16.0 Hz, 1H), 6.92 (d, J = 16.0 Hz, 1H), 4.45 (t, J = 8.0, 2H), 4.40 (t, J = 8.0, 2H), 3.96 (t, J = 5.6 Hz, 2H), 3.76 (t, J = 5.6 Hz, 2H), 3.75 (s, 3H), 3.74 (s, 1H), 2.41 (s, 2H), 2.39 (s, 3H) |
| 23 | 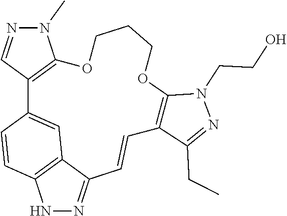 [SMILES: CCc1nn(CCO)c2c1/C=C/c1n[nH]c3ccc(cc13)-c1cnn(C)c1OCCCO2] | 435.1 | 13.13-12.76 (m, 1H), 8.57 (s, 1H), 7.91 (s, 1H), 7.72-7.64 (m, 1H), 7.53-7.39 (m, 2H), 6.88 (d, J = 17.2 Hz, 1H), 4.45 (br t, J = 5.2 Hz, 2H), 4.40 (br t, J = 4.8 Hz, 2H), 3.97 (br t, J = 5.6 Hz, 2H), 3.78-3.75 (m, 3H), 3.75 (s, 3H), 2.82-2.75 (m, 2H), 2.40 (br d, J = 4.4 Hz, 2H), 1.27 (t, J = 7.6 Hz, 3H) |
| 24 | 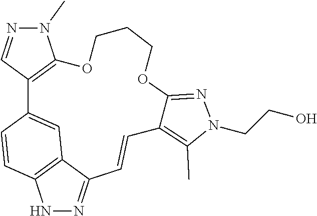 [SMILES: Cc1c2c(nn1CCO)OCCCOc1c(cnn1C)-c1ccc3[nH]nc(c3c1)/C=C/2] | 420.8 | 12.92 (s, 1H), 8.47 (s, 1H), 7.89 (s, 1H), 7.71-7.65 (d, J = 8.4 Hz, 1H), 7.50 (d, J = 8.4 Hz, 1H), 7.41 (d, J = 17.6 Hz, 1H), 6.97-6.89 (d, J = 17.6 Hz, 1H), 4.91 (t, J = 5.6 Hz, 1H), 4.41 (t, J = 6.0 Hz, 2H), 4.27 (t, J = 5.2 Hz, 2H), 4.04 (t, J = 5.6 Hz, 2H), 3.73 (s, 3H), 3.72-3.66 (m, 2H), 2.50 (s, 3H), 2.32-2.26 (m, 2H) |
| 25 | 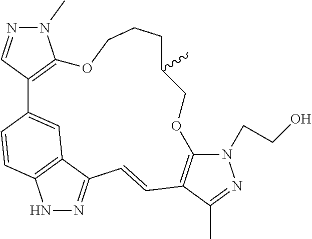 [SMILES: Cc1nn(CCO)c2c1/C=C/c1n[nH]c3ccc(cc13)-c1cnn(C)c1OCCCC(C)CO2] | 463.0 | 8.12 (s, 1H), 7.80-7.76 (m, 1H), 7.60-7.56 (m, 1H), 7.53- 7.49 (m, 1H), 7.23-7.11 (m, 2H), 4.06 (br dd, J = 2.3, 8.6 Hz, 1H), 4.02 (br d, J = 2.9 Hz, 1H), 3.99 (br s, 1H), 3.94 (br s, 1H), 3.88 (br d, J = 6.5 Hz, 1H), 3.86 (br d, J = 6.6 Hz, 1H), 3.76 (br s, 2H), 3.72 (s, 3H), 2.24 (s, 3H), 2.22-2.16 (m, 1H), 2.02- 1.94 (m, 2H), 1.80-1.74 (m, 2H), 1.07 (d, J = 6.9 Hz, 3H) |
| 26 | 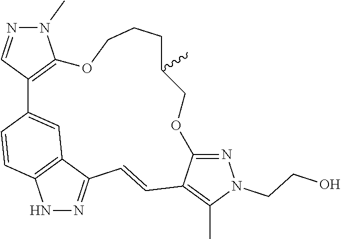 [SMILES: Cc1c2c(nn1CCO)OCC(C)CCCOc1c(cnn1C)-c1ccc3[nH]nc(c3c1)/C=C/2] | 463.1 | 8.15-8.05 (m, 1H), 7.91-7.79 (m, 1H), 7.69-7.58 (m, 1H), 7.53-7.36 (m, 2H), 7.15 (d, J = 16.1 Hz, 1H), 4.37 (dd, J = 4.1, 9.8 Hz, 1H), 4.15-4.09 (m, 1H), 4.06-4.01 (m, 1H), 3.97-3.94 (m, 2H), 3.93 (br d, J = 4.6 Hz, 1H), 3.71 (s, 3H), 3.67-3.66 (m, 2H), 2.30 (s, 3H), 2.28-2.22 (m, 1H), 2.07- 1.95 (m, 2H), 1.82-1.75 (m, 1H), 1.74-1.65 (m, 1H), 1.01 (d, J = 6.8 Hz, 3H) |
| 27 | 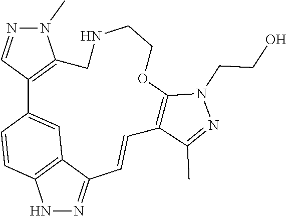 [SMILES: Cc1nn(CCO)c2c1/C=C/c1n[nH]c3ccc(cc13)-c1cnn(C)c1CNCCO2] | 419.9 | 13.05 (br s, 1H), 9.69-9.56 (m, 1H), 8.36-8.09 (m, 1H), 7.98-7.84 (m, 1H), 7.58 (br s, 2H), 7.55 (s, 1H), 7.01-6.93 (m, 1H), 5.07-4.85 (m, 1H), 4.73-4.48 (m, 4H), 4.02 (br d, J = 16.3 Hz, 5H), 3.80-3.75 (m, 2H), 3.71-3.60 (m, 2H), 2.40 (s, 3H) |
| 28 | 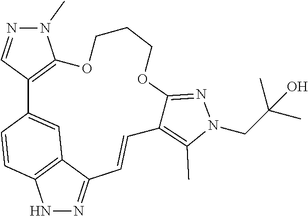 [SMILES: Cc1c2c(nn1CC(C)(C)O)OCCCOc1c(cnn1C)-c1ccc3[nH]nc(c3c1)/C=C/2] | 449.3 | 8.48 (s, 1H), 7.89 (s, 1H), 7.67 (d, J = 8.8 Hz, 1H), 7.49 (d, J = 8.8 Hz, 1H), 7.42 (d, J = 17.6 Hz, 1H), 6.93 (d, J = 17.6 Hz, 1H), 4.41 (t, J = 6.0 Hz, 2H), 4.27 (t, J = 6.0 Hz, 2H), 3.91 (s, 2H), 3.73 (s, 3H), 3.65 (s, 1H), 3.41-3.39 (m, 3H), 2.32- 2.26 (m, 2H), 1.13 (s, 6H) |
| 29 | 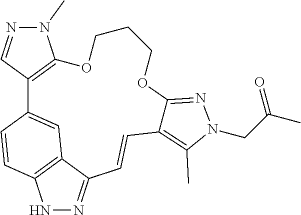 [SMILES: CC(=O)Cn1nc2c(c1C)/C=C/c1n[nH]c3ccc(cc13)-c1cnn(C)c1OCCCO2] | 433.2 | 8.47 (s, 1H), 7.89 (s, 1H), 7.67 (d, J = 8.4, 1H), 7.49 (d, J = 8.4 Hz, 1H), 7.40 (d, J = 17.6 Hz, 1H), 6.94 (d, J = 17.6 Hz, 1H), 5.05 (s, 2H), 4.41 (t, J = 6.0 Hz, 2H), 4.25 (t, J = 6.0 Hz, 2H), 3.72 (s, 3H), 2.37 (s, 3H), 2.31-2.25 (m, 2H), 2.15 (s, 3H) |
| 30 | 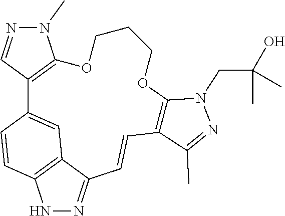 [SMILES: Cc1nn(CC(C)(C)O)c2c1/C=C/c1n[nH]c3ccc(cc13)-c1cnn(C)c1OCCCO2] | 449.3 | 8.57 (s, 1H), 7.80 (s, 1H), 7.62 (d, J = 8.4 Hz, 1H), 7.52-7.42 (m, 2H), 6.93 (d, J = 16.8 Hz, 1H), 4.39 (d, J = 3.6 Hz, 4H), 3.90 (s, 2H), 3.73 (s, 3H), 2.44 (s, 3H), 2.41-2.35 (m, 2H), 1.23 (s, 6H) |
| 31 | 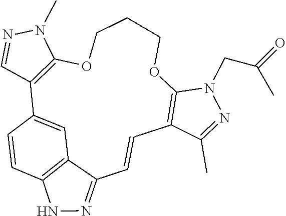 | 433.1 | 8.71-8.66 (s, 1H), 7.87 (s, 1H), 7.72 (s, 1H), 7.61-7.58 (d, J = 8.0 Hz, 1H), 7.52-7.49 (d, J = 8.0 Hz, 1H), 7.08-6.99 (d, J = 16.0 Hz, 1H), 4.62 (s, 2H), 4.53-4.46 (m, 2H), 4.45- 4.38 (m, 2H), 3.81-3.77 (s, 3H), 2.49-2.46 (s, 3H), 2.22 (s, 2H), 1.95-1.90 (s, 3H) |
| 32 | 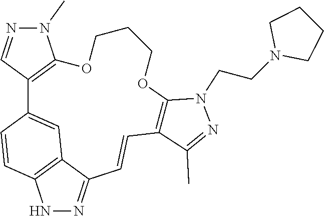 [SMILES: Cc1nn(CCN2CCCC2)c2c1/C=C/c1n[nH]c3ccc(cc13)-c1cnn(C)c1OCCCO2] | 474.0 | 13.03 (s, 1H), 9.74-9.63 (s, 1H), 8.60-8.56 (s, 1H), 7.95- 7.91 (s, 1H), 7.74-7.67 (d, J = 7.2 Hz, 1H), 7.55-7.49 (d, J = 8.8 Hz, 1H), 7.46 (d, J = 17.2 Hz, 1H), 7.02-6.95 (d, J = 17.2 Hz, 1H), 4.47 (t, J = 5.2 Hz, 2H), 4.43 (t, J = 5.2 Hz, 2H), 4.34 (t, J = 6.4 Hz, 2H), 3.77 (s, 3H), 3.64 (d, J = 6.0 Hz, 4H), 3.60 (s, 2H), 3.14- 3.04 (m, 2H), 2.43 (s, 3H), 2.06-1.98 (m, 2H), 1.92-1.83 (m, 2H) |
| 33 | 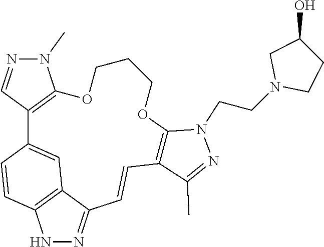 [SMILES: Cc1nn(CCN2CC[C@H](O)C2)c2c1/C=C/c1n[nH]c3ccc(cc13)-c1cnn(C)c1OCCCO2] | 504.2 | 13.13-12.84 (m, 1H), 8.59- 8.55 (m, 1H), 7.93 (s, 1H), 7.72-7.67 (m, 1H), 7.51 (d, J = 8.8 Hz, 1H), 7.44 (d, J = 17.2 Hz, 1H), 6.94 (d, J = 17.2 Hz, 1H), 4.51-4.33 (m, 8H), 3.76 (s, 3H), 3.46 (br d, J = 12.8 Hz, 2H), 3.21- 3.10 (m, 2H), 3.09-2.99 (m, 1H), 2.85-2.79 (m, 2H), 2.45- 2.41 (m, 2H), 2.29-2.21 (m, 1H), 2.02-1.79 (m, 2H), 1.29 (t, J = 7.2 Hz, 3H) |
| 34 | 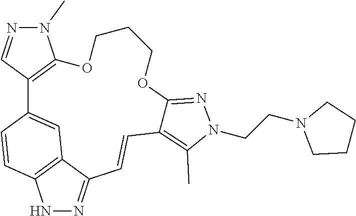 [SMILES: Cc1c2c(nn1CCN1CCCC1)OCCCOc1c(cnn1C)-c1ccc3[nH]nc(c3c1)/C=C/2] | 474.6 | 9.77 (s, 1H), 8.48 (s, 1H), 7.90 (s, 1H), 7.69 (d, J = 8.8 Hz, 1H), 7.51 (d, J = 8.8 Hz, 1H), 7.41 (d, J = 17.2 Hz, 1H), 6.99 (d, J = 17.2 Hz, 1H), 4.42- 4.39 (m, 4H), 4.31 (t, J = 5.2 Hz, 2H), 3.73 (s, 3H), 3.62 (d, J = 5.2 Hz, 4H), 3.12-3.00 (m, 2H), 2.55 (s, 3H), 2.32 (d, J = 5.6 Hz, 2H), 2.07-1.98 (m, 2H), 1.93-1.81 (m, 2H) |
| 35 | 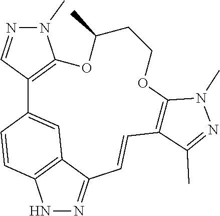 [SMILES: Cc1nn(C)c2c1/C=C/c1n[nH]c3ccc(cc13)-c1cnn(C)c1O[C@@H](C)CCO2] | 404.93 | (500 MHz, DMSO-d6) δ = 13.02-12.77 (m, 1H), 8.40 (s, 1H), 7.91 (s, 1H), 7.69 (d, J = 8.6 Hz, 1H), 7.49 (d, J = 8.6 Hz, 1H), 7.36 (d, J = 17.2 Hz, 1H), 6.91 (d, J = 17.8 Hz, 1H), 4.98-4.92 (m, 1H), 4.43-4.34 (m, 1H), 4.30-4.24 (m, 1H), 3.67 (d, J = 5.7 Hz, 6H), 2.47 (s, 4H), 2.25 (q, J = 5.2 Hz, 2H), 1.28-1.19 (m, 5H) |
| 36 | 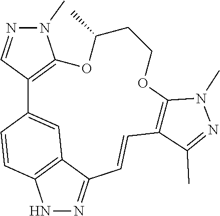 [SMILES: Cc1nn(C)c2c1/C=C/c1n[nH]c3ccc(cc13)-c1cnn(C)c1O[C@H](C)CCO2] | 405.0 | (500 MHz, DMSO-d6) δ = 8.55 (s, 1H), 7.93 (s, 1H), 7.73- 7.68 (m, 1H), 7.49 (d, J = 8.6 Hz, 1H), 7.40 (d, J = 17.8 Hz, 1H), 7.28 (br d, J = 4.0 Hz, 1H), 6.90 (d, J = 17.2 Hz, 1H), 6.68 (br d, J = 5.2 Hz, 1H), 6.55 (br s, 1H), 4.92-4.85 (m, 1H), 4.60-4.53 (m, 1H), 4.34- 4.29 (m, 1H), 3.69 (s, 3H), 3.63 (s, 3H), 2.41-2.35 (m, 5H), 1.30 (d, J = 6.3 Hz, 3H) |
| 37 | 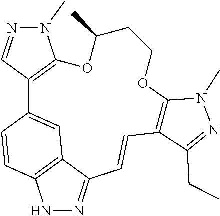 [SMILES: CCc1nn(C)c2c1/C=C/c1n[nH]c3ccc(cc13)-c1cnn(C)c1O[C@@H](C)CCO2] | 419.00 | (500 MHz, DMSO-d6) δ = 13.05-12.78 (m, 1H), 8.55 (s, 1H), 7.93 (s, 1H), 7.70 (dd, J = 1.4, 8.8 Hz, 1H), 7.49 (d, J = 8.8 Hz, 1H), 7.39 (d, J = 17.2 Hz, 1H), 6.86 (d, J = 17.2 Hz, 1H), 4.93-4.84 (m, 1H), 4.57 (ddd, J = 3.3, 6.8, 9.9 Hz, 1H), 4.32 (ddd, J = 3.4, 6.2, 9.8 Hz, 1H), 3.69 (s, 3H), 3.65 (s, 3H), 2.77 (q, J = 7.5 Hz, 2H), 2.41-2.35 (m, 2H), 1.30 (d, J = 6.6 Hz, 3H), 1.26 (t, J = 7.4 Hz, 3H) |
| 38 | 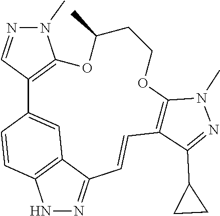 [SMILES: C[C@H]1CCOc2c(c(C3CC3)nn2C)/C=C/c2n[nH]c3ccc(cc23)-c2cnn(C)c2O1] | 432.27 | 8.42 (s, 1H), 7.90 (s, 1H), 7.69 (dd, J = 1.1, 8.7 Hz, 1H), 7.49 (d, J = 8.9 Hz, 1H), 7.35 (d, J = 17.4 Hz, 1H), 7.09 (d, J = 17.2 Hz, 1H), 4.94 (q, J = 5.7 Hz, 1H), 4.43-4.36 (m, 1H), 4.28- 4.21 (m, 1H), 3.77 (s, 3H), 3.67 (s, 3H), 2.25 (q, J = 5.2 Hz, 2H), 1.96-1.90 (m, 1H), 1.21 (d, J = 6.4 Hz, 3H), 1.16 (dd, J = 1.4, 8.2 Hz, 2H), 0.80- 0.73 (m, 2H) |
| 39 | 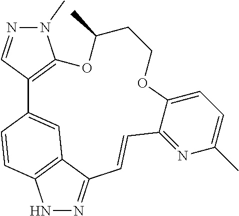 [SMILES: Cc1ccc2c(n1)/C=C/c1n[nH]c3ccc(cc13)-c1cnn(C)c1O[C@@H](C)CCO2] | 401.6 | 8.48 (s, 1H), 8.06 (d, J = 16.7 Hz, 1H), 7.93-7.87 (m, 2H), 7.88-7.81 (m, 1H), 7.74 (dd, J = 1.1, 8.8 Hz, 1H), 7.59 (d, J = 8.5 Hz, 1H), 7.36 (br d, J = 7.7 Hz, 1H), 5.10 (qd, J = 5.9, 11.7 Hz, 1H), 4.51 (br s, 1H), 4.31 (br s, 1H), 3.69 (s, 3H), 2.56 (s, 3H), 2.38-2.31 (m, 2H), 1.20 (d, J = 6.3 Hz, 3H) |
| 40 | 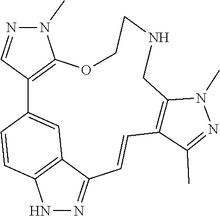 [SMILES: Cc1nn(C)c2c1/C=C/c1n[nH]c3ccc(cc13)-c1cnn(C)c1OCCNC2] | 390.2 | 12.85 (s, 1H), 8.61 (s, 1H), 8.12 (d, J = 17.6 Hz, 1H), 7.91 (s, 1H), 7.69 (d, J = 8.8 Hz, 1H), 7.48 (d, J = 8.8 Hz, 1H), 6.90 (d, J = 17.6 Hz, 1H), 4.21 (s, 2H), 3.96 (s, 2H), 3.73 (s, 6H), 3.10 (d, J = 1.2 Hz, 2H), 2.39 (s, 3H) |
| 41 | 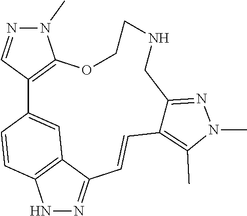 [SMILES: Cc1c2c(nn1C)CNCCOc1c(cnn1C)-c1ccc3[nH]nc(c3c1)/C=C/2] | 389.9 | 13.12-12.90 (m, 1H), 9.41 (br d, J = 4.4 Hz, 1H), 8.61 (s, 1H), 7.95 (s, 1H), 7.76-7.68 (m, 1H), 7.58-7.50 (m, 2H), 7.03 (d, J = 17.2 Hz, 1H), 4.59 (br s, 2H), 4.44 (br s, 2H), 3.85 (s, 3H), 3.77 (s, 3H), 3.62 (br s, 2H), 2.55 (s, 3H) |
| 42 | 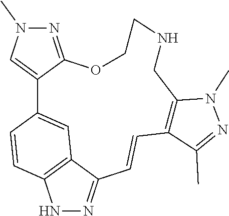 | 436.9 | 13.00-12.88 (m, 1H), 8.80 (s, 1H), 8.06-8.03 (m, 1H), 7.61- 7.43 (m, 3H), 7.02 (d, J = 16.8 Hz, 1H), 4.62 (br s, 4H), 3.87 (s, 3H), 3.76-3.73 (m, 3H), 3.67-3.57 (m, 2H), 2.42 (s, 3H) |
| 43 | 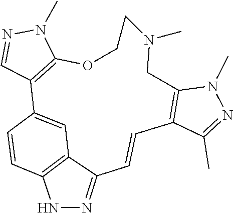 [SMILES: Cc1nn(C)c2c1/C=C/c1n[nH]c3ccc(cc13)-c1cnn(C)c1OCCN(C)C2] | 403.9 | 12.87 (s, 1H), 8.61 (s, 1H), 8.04 (d, J = 17.6 Hz, 1H), 7.95- 7.87 (m, 1H), 7.71-7.65 (m, 1H), 7.47 (d, J = 8.8 Hz, 1H), 6.93 (d, J = 17.6 Hz, 1H), 4.26 (t, J = 3.6 Hz, 2H), 3.82 (s, 2H), 3.74 (d, J = 2.8 Hz, 6H), 2.99 (t, J = 4.0 Hz, 2H), 2.39 (s, 3H), 2.19 (s, 3H) |
| 44 | 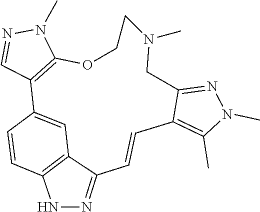 [SMILES: Cc1c2c(nn1C)CN(C)CCOc1c(cnn1C)-c1ccc3[nH]nc(c3c1)/C=C/2] | 403.9 | 8.78 (d, J = 11.2 Hz, 2H), 8.04- 7.95 (m, 1H), 7.91 (s, 1H), 7.87-7.76 (m, 2H), 7.43 (d, J = 8.4 Hz, 1H), 4.40 (t, J = 5.6 Hz, 2H), 4.33-4.23 (m, 2H), 3.77 (s, 3H), 2.67 (s, 3H), 2.49 -2.37 (m, 2H) |
| 45 | 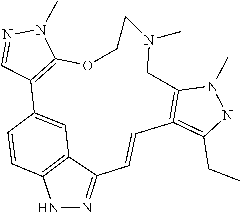 | 418.1 | 8.69 (s, 1H), 7.88 (s, 1H), 7.74- 7.66 (m, 2H), 7.52 (d, J = 8.4 Hz, 1H), 7.13 (d, J = 17.2 Hz, 1H), 4.61 (br s, 3H), 3.94 (s, 3H), 3.82 (s, 3H), 3.81-3.80 (m, 1H), 3.31-3.29 (m, 3H), 2.93 (q, J = 7.6 Hz, 2H), 2.87 (s, 2H), 1.37 (t, J = 7.6 Hz, 3H) |
| 46 | 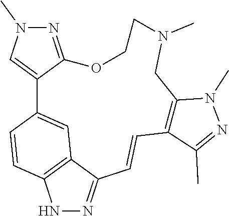 | 404.1 | 9.03-8.95 (m, 1H), 8.18-8.07 (m, 1H), 7.83-7.76 (m, 1H), 7.60-7.54 (m, 1H), 7.47-7.39 (m, 1H), 7.04 (d, J = 17.6 Hz, 1H), 4.41-4.33 (m, 2H), 3.82 (s, 3H), 3.80-3.77 (m, 3H), 3.76 (br s, 2H), 3.00-2.92 (m, 2H), 2.48 (s, 3H), 2.17 (s, 3H) |
| 47 | 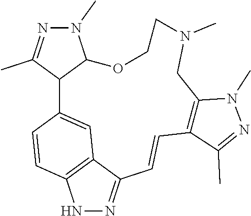 | 418.1 | 8.59 (s, 1H), 7.71-7.65 (m, 1H), 7.62-7.52 (m, 2H), 7.19 (d, J = 16.8 Hz, 1H), 4.84 (br s, 2H), 4.54 (br dd, J = 2.4, 6.0 Hz, 2H), 3.96 (s, 3H), 3.93- 3.88 (m, 2H), 3.79 (s, 3H), 2.93 (s, 3H), 2.55 (s, 3H), 2.45 (s, 3H) |
| 48 | 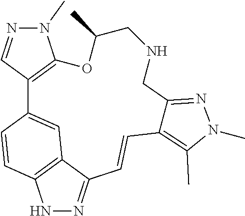 [SMILES: Cc1c2c(nn1C)CNC[C@H](C)Oc1c(cnn1C)-c1ccc3[nH]nc(c3c1)/C=C/2] | 403.97 | (500 MHz, DMSO-d6) δ = 13.01 (br s, 1H), 9.61-9.44 (m, 1H), 9.35-9.19 (m, 1H), 8.51 (s, 1H), 7.89 (s, 1H), 7.68 (br dd, J = 1.4, 8.5 Hz, 1H), 7.59-7.39 (m, 2H), 7.06-6.87 (m, 1H), 5.20-5.07 (m, 1H), 4.67-4.51 (m, 1H), 4.37-4.24 (m, 1H), 3.85 (s, 3H), 3.73 (s, 3H), 3.57-3.51 (m, 1H), 3.46- 3.42 (m, 1H), 2.57-2.54 (m, 3H), 1.12-1.06 (m, 3H) |
| 49 | 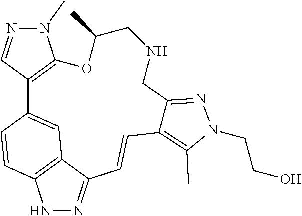 [SMILES: Cc1c2c(nn1CCO)CNC[C@H](C)Oc1c(cnn1C)-c1ccc3[nH]nc(c3c1)/C=C/2] | 434.0 | 13.01 (br s, 1H), 9.71-9.47 (m, 1H), 9.39-9.11 (m, 1H), 8.51 (s, 1H), 8.03-7.83 (m, 1H), 7.68 (dd, J = 1.4, 8.8 Hz, 1H), 7.58-7.41 (m, 2H), 7.00 (d, J = 17.0 Hz, 1H), 5.24- 5.07 (m, 1H), 5.04-4.84 (m, 1H), 4.65-4.53 (m, 1H), 4.39- 4.26 (m, 1H), 4.26-4.06 (m, 2H), 3.76 (br t, J = 5.5 Hz, 2H), 3.72 (s, 3H), 2.89 (s, 1H), 2.77-2.72 (m, 2H), 2.56 (s, 2H), 1.09 (d, J = 6.3 Hz, 3H) |
| 50 | 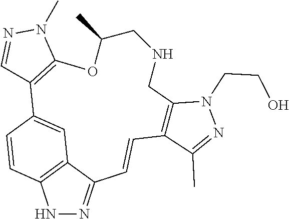 [SMILES: Cc1nn(CCO)c2c1/C=C/c1n[nH]c3ccc(cc13)-c1cnn(C)c1O[C@@H](C)CNC2] | 434.0 | |
| 51 | 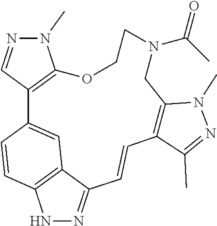 [SMILES: CC(=O)N1CCOc2c(cnn2C)-c2ccc3[nH]nc(c3c2)/C=C/c2c(C)nn(C)c2C1] | 432.1 | 12.95 (s, 1H), 8.40 (s, 1H), 7.92 (s, 1H), 7.68 (d, J = 8.8 Hz, 1H), 7.57 (d, J = 16.8 Hz, 1H), 7.50 (d, J = 8.8 Hz, 1H), 7.06 (d, J = 16.8 Hz, 1H), 4.86 (s, 2H), 4.50 (t, J = 7.6 Hz, 2H), 3.77 (s, 3H), 3.73 (s, 4H), 3.28 (s, 1H), 2.43 (s, 3H), 2.23 (s, 3H) |
| 52 | 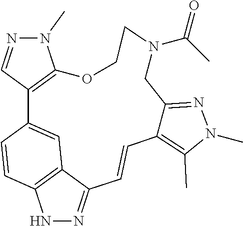 [SMILES: CC(=O)N1CCOc2c(cnn2C)-c2ccc3[nH]nc(c3c2)/C=C/c2c(nn(C)c2C)C1] | 431.9 | 13.39-12.60 (m, 1H), 8.51 (s, 1H), 7.89 (s, 1H), 7.68-7.61 (m, 2H), 7.51 (d, J = 8.4 Hz, 1H), 7.05 (br d, J = 17.2 Hz, 1H), 4.63 (s, 2H), 4.33 (br t, J = 7.2 Hz, 2H), 3.80 (s, 3H), 3.77 (br s, 2H), 3.72 (s, 3H), 2.54 (s, 3H), 2.40 (s, 3H) |
| 53 | 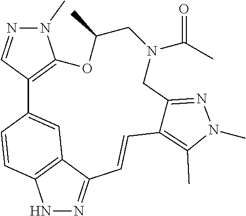 [SMILES: CC(=O)N1Cc2nn(C)c(C)c2/C=C/c2n[nH]c3ccc(cc23)-c2cnn(C)c2O[C@@H](C)C1] | 445.94 | (500 MHz, DMSO-d6) δ = 13.06-12.89 (m, 1H), 8.65- 8.59 (m, 1H), 7.99-7.96 (m, 1H), 7.74-7.70 (m, 1H), 7.65- 7.57 (m, 1H), 7.53-7.48 (m, 1H), 7.07-7.00 (m, 1H), 5.05- 4.93 (m, 1H), 4.79-4.65 (m, 1H), 4.63-4.50 (m, 1H), 4.14- 4.00 (m, 1H), 3.80 (s, 4H), 3.72-3.66 (m, 3H), 2.53 (s, 3H), 2.48-2.41 (m, 3H), 1.14- 1.05 (m, 3H) |
| 54 | 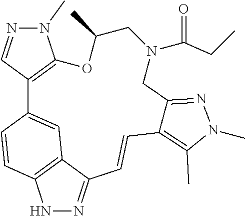 [SMILES: CCC(=O)N1Cc2nn(C)c(C)c2/C=C/c2n[nH]c3ccc(cc23)-c2cnn(C)c2O[C@@H](C)C1] | 459.98 | (499 MHz, DMSO-d6) δ = 13.03-12.91 (m, 1H), 8.70- 8.57 (m, 1H), 7.97 (s, 1H), 7.70 (s, 1H), 7.65-7.59 (m, 1H), 7.51 (d, J = 8.8 Hz, 1H), 7.03 (d, J = 17.0 Hz, 1H), 5.03- 4.95 (m, 1H), 4.76-4.69 (m, 1H), 4.60-4.50 (m, 1H), 4.44- 4.36 (m, 1H), 4.10-4.03 (m, 1H), 3.82-3.78 (m, 3H), 3.70- 3.66 (m, 3H), 3.04-2.90 (m, 1H), 2.88-2.76 (m, 1H), 2.55- 2.53 (m, 3H), 1.25-1.19 (m, 2H), 1.10-1.08 (m, 3H), 1.03 -0.98 (m, 1H) |
| 55 | 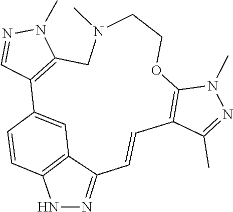 [SMILES: Cc1nn(C)c2c1/C=C/c1n[nH]c3ccc(cc13)-c1cnn(C)c1CN(C)CCO2] | 403.8 | 13.55-12.31 (m, 1H), 8.67- 8.32 (m, 1H), 8.02-7.88 (m, 1H), 7.65-7.59 (m, 1H), 7.58- 7.52 (m, 1H), 7.48 (d, J = 17.2 Hz, 1H), 6.95 (d, J = 17.2 Hz, 1H), 4.75-4.23 (m, 4H), 3.98 (s, 3H), 3.64 (s, 3H), 2.67 (s, 2H), 2.50 (s, 3H), 2.36 (s, 3H) |
| 56 | 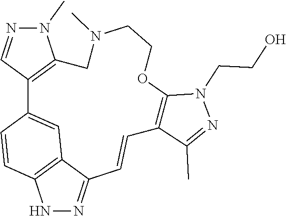 [SMILES: Cc1nn(CCO)c2c1/C=C/c1n[nH]c3ccc(cc13)-c1cnn(C)c1CN(C)CCO2] | 434.1 | 13.35-12.65 (m, 1H), 8.89- 8.62 (m, 1H), 8.00-7.75 (m, 1H), 7.65-7.52 (m, 2H), 7.49 (d, J = 17.6 Hz, 1H), 7.00- 6.90 (m, 1H), 4.97-4.72 (m, 1H), 4.54-4.35 (m, 2H), 4.00- 3.89 (m, 6H), 3.74 (s, 3H), 3.07-2.93 (m, 2H), 2.54-2.52 (m, 3H), 2.38 (s, 3H), 2.30- 2.20 (m, 2H) |
| 57 | 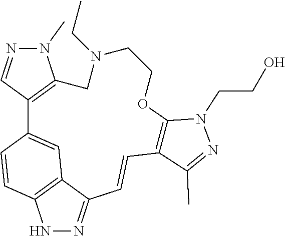 [SMILES: CCN1CCOc2c(c(C)nn2CCO)/C=C/c2n[nH]c3ccc(cc23)-c2cnn(C)c2C1] | 448.1 | 13.10-12.89 (m, 1H), 8.72 (br s, 1H), 7.95-7.76 (m, 1H), 7.65-7.58 (m, 1H), 7.56-7.49 (m, 2H), 6.98-6.92 (m, 1H), 4.52-4.36 (m, 2H), 4.07-3.88 (m, 9H), 3.78-3.69 (m, 3H), 2.38 (s, 3H), 1.38-1.14 (m, 2H), 1.10-0.92 (m, 3H) |
| 58 | 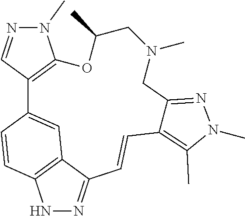 [SMILES: Cc1c2c(nn1C)CN(C)C[C@H](C)Oc1c(cnn1C)-c1ccc3[nH]nc(c3c1)/C=C/2] | 417.99 | (500 MHz, DMSO-d6) δ = 13.06-12.89 (m, 1H), 8.65- 8.59 (m, 1H), 7.99-7.96 (m, 1H), 7.74-7.70 (m, 1H), 7.65- 7.57 (m, 1H), 7.53-7.48 (m, 1H), 7.07-7.00 (m, 1H), 5.05- 4.93 (m, 1H), 4.79-4.65 (m, 1H), 4.63-4.50 (m, 1H), 4.14- 4.00 (m, 1H), 3.80 (s, 4H), 3.72-3.66 (m, 3H), 2.53 (s, 3H), 2.48-2.41 (m, 3H), 1.14- 1.05 (m, 3H) |
| 59 | 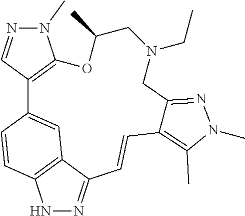 [SMILES: CCN1Cc2nn(C)c(C)c2/C=C/c2n[nH]c3ccc(cc23)-c2cnn(C)c2O[C@@H](C)C1] | 432.00 | (500 MHz, DMSO-d6) δ = 7.57 (s, 1H), 7.49-7.46 (m, 1H), 7.41-7.38 (m, 1H), 7.26- 7.23 (m, 1H), 6.79 (d, J = 12.0 Hz, 1H), 6.62-6.58 (m, 1H), 4.26-4.19 (m, 1H), 4.17- 4.12 (m, 1H), 3.84-3.79 (m, 1H), 3.70-3.67 (m, 1H), 3.66 (s, 3H), 3.44-3.42 (m, 3H), 1.82-1.66 (m, 3H), 1.57-1.47 (m, 1H), 1.43 (s, 3H), 1.27- 1.23 (m, 1H), 0.84 (d, J = 6.6 Hz, 3H) |
| 60 | 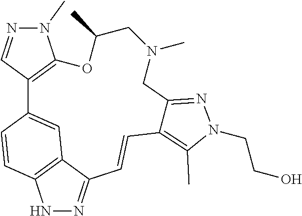 [SMILES: Cc1c2c(nn1CCO)CN(C)C[C@H](C)Oc1c(cnn1C)-c1ccc3[nH]nc(c3c1)/C=C/2] | 448.0 | (500 MHz, DMSO-d6) δ = 8.49-8.46 (m, 1H), 7.94-7.90 (m, 1H), 7.85-7.82 (m, 1H), 7.68-7.65 (m, 1H), 7.56-7.53 (m, 1H), 7.02-6.97 (m, 1H), 4.59 (s, 1H), 4.22-4.16 (m, 2H), 3.78-3.74 (m, 2H), 3.72- 3.68 (m, 3H), 3.11-3.04 (m, 2H), 2.88 (s, 2H), 2.75-2.73 (m, 3H), 2.55-2.53 (m, 3H), 2.39-2.37 (m, 1H), 1.09-1.04 (m, 3H) |
| 61 | 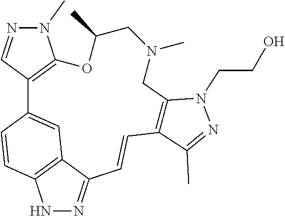 [SMILES: Cc1nn(CCO)c2c1/C=C/c1n[nH]c3ccc(cc13)-c1cnn(C)c1O[C@@H](C)CN(C)C2] | 448.0 | (500 MHz, DMSO-d6) δ = 8.55-8.42 (m, 1H), 7.87-7.78 (m, 1H), 7.74-7.68 (m, 1H), 7.68-7.63 (m, 1H), 7.58-7.53 (m, 1H), 7.04-6.97 (m, 1H), 5.18-5.04 (m, 1H), 4.66-4.51 (m, 1H), 4.41-4.28 (m, 1H), 4.25-4.14 (m, 2H), 3.68 (s, 5H), 3.54-3.46 (m, 1H), 3.44- 3.36 (m, 1H), 2.74-2.66 (m, 3H), 2.43 (s, 3H), 1.09-1.01 (m, 3H) |
| 62 | 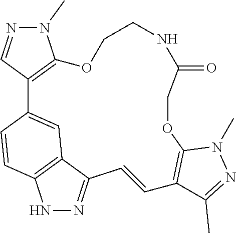 [SMILES: Cc1nn(C)c2c1/C=C/c1n[nH]c3ccc(cc13)-c1cnn(C)c1OCCNC(=O)CO2] | 433.9 | 9.06 (t, J = 6.0 Hz, 1H), 8.13 (s, 1H), 7.85 (s, 1H), 7.64 (dd, J = 1.2, 8.8 Hz, 1H), 7.49 (d, J = 8.8 Hz, 1H), 7.18-7.13 (m, 1H), 7.13-7.07 (m, 1H), 4.58 (s, 2H), 4.09 (t, J = 4.0 Hz, 2H), 3.74 (s, 5H), 3.66 (s, 3H), 2.23 (s, 3H) |
| 63 | 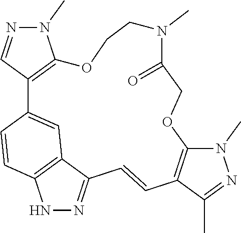 [SMILES: Cc1nn(C)c2c1/C=C/c1n[nH]c3ccc(cc13)-c1cnn(C)c1OCCN(C)C(=O)CO2] | 447.9 | 8.32-7.86 (m, 1H), 7.73 (s, 1H), 7.60-7.55 (m, 1H), 7.54- 7.44 (m, 1H), 7.34-7.07 (m, 1H), 7.03-6.86 (m, 1H), 5.15- 4.95 (m, 2H), 4.23-4.15 (m, 2H), 3.89-3.83 (m, 2H), 3.78- 3.71 (m, 3H), 3.65 (br s, 3H), 3.13-3.00 (m, 3H), 2.37-2.20 (m, 3H) |
| 64 | 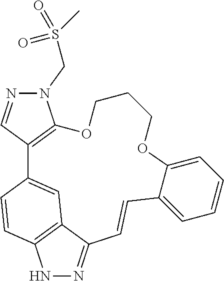 [SMILES: CS(=O)(=O)Cn1ncc2c1OCCCOc1ccccc1/C=C/c1n[nH]c3ccc-2cc13] | 450.9 | 8.55 (s, 1H), 8.23 (d, J = 17.2 Hz, 1H), 7.99 (s, 1H), 7.80 (d, J = 6.4 Hz, 1H), 7.73- 7.70 (m, 1H), 7.56 (d, J = 8.4 Hz, 1H), 7.38-7.32 (m, 1H), 7.19 (d, J = 8.4 Hz, 1H), 7.08 (t, J = 7.6 Hz, 1H), 5.47 (s, 2H), 4.68 (t, J = 5.6 Hz, 2H), 4.34 (t, J = 5.6 Hz, 2H), 3.13 (s, 3H), 2.42-2.38 (m, 2H) |
| 65 | 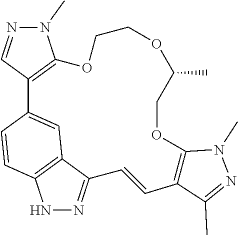 [SMILES: Cc1nn(C)c2c1/C=C/c1n[nH]c3ccc(cc13)-c1cnn(C)c1OCCO[C@H](C)CO2] | 435.1 | 1H NMR (400 MHz, DMSO- d6) δ = 12.94 (s, 1H), 8.24 (s, 1H), 7.83 (s, 1H), 7.62 (d, J = 8.0 Hz, 1H), 7.48 (d, J = 8.0 Hz, 1H), 7.26-7.21 (m, 1H), 7.20-7.15 (m, 1H), 4.27-4.20 (m, 1H), 4.19-4.09 (m, 3H), 4.08-3.98 (m, 3H), 3.73 (s, 3H), 3.63 (s, 3H), 2.23 (s, 3H), 1.32 (br d, J = 6.0 Hz, 3H) |
| 66 | 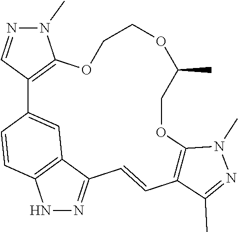 [SMILES: Cc1nn(C)c2c1/C=C/c1n[nH]c3ccc(cc13)-c1cnn(C)c1OCCO[C@@H](C)CO2] | 435.2 | 1H NMR (400 MHz, DMSO- d6) δ = 12.94 (s, 1H), 8.24 (s, 1H), 7.83 (s, 1H), 7.62 (d, J = 8.0 Hz, 1H), 7.48 (d, J = 8.0 Hz, 1H), 7.26-7.21 (m, 1H), 7.20-7.15 (m, 1H), 4.27-4.20 (m, 1H), 4.19-4.09 (m, 3H), 4.08-3.98 (m, 3H), 3.73 (s, 3H), 3.63 (s, 3H), 2.23 (s, 3H), 1.32 (br d, J = 6.0 Hz, 3H) |
| 67 | 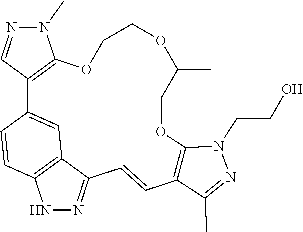 [SMILES: Cc1nn(CCO)c2c1/C=C/c1n[nH]c3ccc(cc13)-c1cnn(C)c1OCCOC(C)CO2] | 464.1 | 1H NMR (400 MHz, DMSO- d6) δ = 8.23 (s, 1H), 7.78 (s, 1H), 7.64-7.56 (m, 1H), 7.53- 7.44 (m, 1H), 7.30-7.20 (m, 1H), 7.18-7.10 (m, 1H), 4.63 (br dd, J = 4.8, 10.0 Hz, 1H), 4.22-4.09 (m, 2H), 4.02-3.80 (m, 5H), 3.72 (s, 3H), 2.26 (s, 3H), 1.38-1.30 (m, 1H), 1.28- 1.22 (m, 3H), 1.16 (d, J = 6.0 Hz, 3H) |
| 68 | 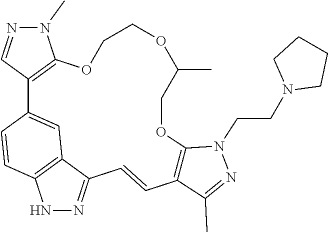 [SMILES: Cc1nn(CCN2CCCC2)c2c1/C=C/c1n[nH]c3ccc(cc13)-c1cnn(C)c1OCCOC(C)CO2] | 518.4 | 1H NMR (400 MHz, DMSO- d6) δ = 13.84-11.86 (m, 1H), 8.28-8.18 (m, 1H), 7.86-7.75 (m, 1H), 7.66-7.57 (m, 1H), 7.54-7.47 (m, 1H), 7.30-7.22 (m, 1H), 7.21-7.14 (m, 1H), 4.66 (br t, J = 6.0 Hz, 1H), 4.37-4.05 (m, 6H), 4.02-3.93 (m, 2H), 3.73 (s, 3H), 3.67- 3.62 (m, 4H), 3.08 (br d, J = 6.0 Hz, 2H), 2.33-2.27 (m, 3H), 2.03 (br s, 2H), 1.87 (br d, J = 6.0 Hz, 2H), 1.18 (d, J = 6.4 Hz, 3H) |
| 69 | 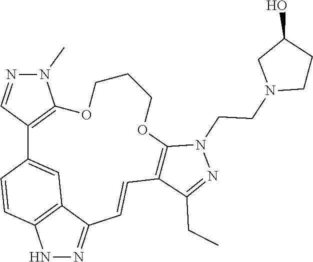 [SMILES: CCc1nn(CCN2CC[C@H](O)C2)c2c1/C=C/c1n[nH]c3ccc(cc13)-c1cnn(C)c1OCCCO2] | 504.2 | 1H NMR (400 MHz, DMSO- d6) δ = 13.13-12.84 (m, 1H), 8.59-8.55 (m, 1H), 7.93 (s, 1H), 7.72-7.67 (m, 1H), 7.51 (d, J = 8.8 Hz, 1H), 7.44 (d, J = 17.2 Hz, 1H), 6.94 (d, J = 17.2 Hz, 1H), 4.51-4.33 (m, 8H), 3.76 (s, 3H), 3.46 (br d, J = 12.8 Hz, 2H), 3.21-3.10 (m, 2H), 3.09-2.99 (m, 1H), 2.85- 2.79 (m, 2H), 2.45-2.41 (m, 2H), 2.29-2.21 (m, 1H), 2.02- 1.79 (m, 2H), 1.29 (t, J = 7.2 Hz, 3H) |
| 70 | 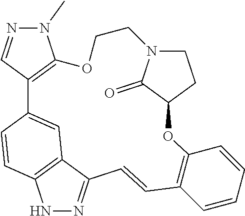 [SMILES: Cn1ncc2c1OCCN1CC[C@@H](Oc3ccccc3/C=C/c3n[nH]c4ccc-2cc34)C1=O] | 442.2 | 1H NMR (400 MHz, METHANOL-d4) δ = 8.19 (s, 1H), 7.77-7.71 (m, 1H), 7.70- 7.65 (m, 2H), 7.64-7.59 (m, 1H), 7.59-7.52 (m, 2H), 7.36- 7.28 (m, 1H), 7.18 (d, J = 8.0 Hz, 1H), 7.10 (t, J = 7.5 Hz, 1H), 5.05 (br d, J = 5.9 Hz, 1H), 4.45-4.38 (m, 1H), 4.36- 4.26 (m, 1H), 4.15-4.04 (m, 2H), 3.85 (s, 3H), 3.57 (br t, J = 8.8 Hz, 1H), 3.46-3.40 (m, 1H), 2.45-2.32 (m, 1H), 2.27- 2.18 (m, 1H) |
| 71 | 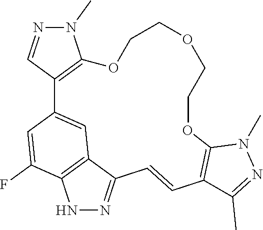 [SMILES: Cc1nn(C)c2c1/C=C/c1n[nH]c3c(F)cc(cc13)-c1cnn(C)c1OCCOCCO2] | 439.0 | 1H NMR (400 MHz, DMSO- d6) δ = 14.57-12.02 (m, 1H), 8.07 (s, 1H), 7.91 (s, 1H), 7.53 (d, J = 12.8 Hz, 1H), 7.23 (s, 2H), 4.26-4.16 (m, 4H), 3.89 (br s, 4H), 3.73 (s, 3H), 3.63 (s, 3H), 2.22 (s, 3H) |
| 72 | 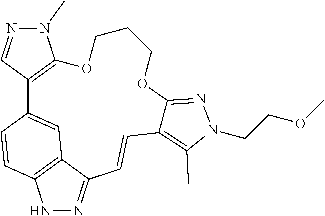 [SMILES: COCCn1nc2c(c1C)/C=C/c1n[nH]c3ccc(cc13)-c1cnn(C)c1OCCCO2] | 435.1 | 1H NMR (400 MHz, DMSO- d6) δ = 12.95-12.85 (m, 1H), 8.47 (s, 1H), 7.89 (s, 1H), 7.71- 7.65 (m, 1H), 7.49 (d, J = 8.8 Hz, 1H), 7.40 (d, J = 17.6 Hz, 1H), 6.93 (d, J = 17.2 Hz, 1H), 4.41 (br t, J = 6.0 Hz, 2H), 4.27 (t, J = 5.2 Hz, 2H), 4.15 (t, J = 5.6 Hz, 2H), 3.73 (s, 3H), 3.64 (t, J = 5.6 Hz, 2H), 3.24 (s, 3H), 2.48 (br s, 3H), 2.32-2.26 (m, 2H) |
| 73 | 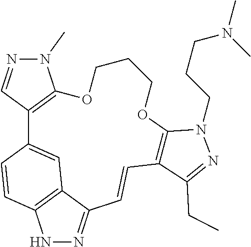 [SMILES: CCc1nn(CCCN(C)C)c2c1/C=C/c1n[nH]c3ccc(cc13)-c1cnn(C)c1OCCCO2] | 476.1 | 1H NMR (400 MHz, METHANOL-d4) δ = 8.12 (s, 1H), 7.72 (s, 2H), 7.68-7.66 (m, 1H), 7.62-7.58 (m, 1H), 7.56-7.52 (m, 1H), 7.28 (d, J = 16.4 Hz, 1H), 6.93 (d, J = 13.2 Hz, 1H), 4.07 (s, 4H), 4.05-3.95 (m, 4H), 3.79 (s, 3H), 2.91-2.89 (m, 6H), 2.85- 2.78 (m, 2H), 2.34-2.24 (m, 2H), 2.22-2.12 (m, 1H), 1.32 (t, J = 7.6 Hz, 3H) |
| 74 | 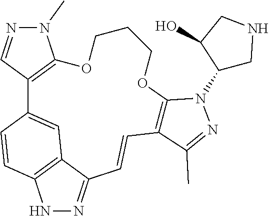 [SMILES: Cc1nn([C@H]2CNC[C@@H]2O)c2c1/C=C/c1n[nH]c3ccc(cc13)-c1cnn(C)c1OCCCO2] | 462.2 | 1H NMR (400 MHz, DMSO- d6) δ = 13.16-12.77 (m, 1H), 8.57 (s, 1H), 7.92 (s, 1H), 7.69 (dd, J = 1.3, 8.8 Hz, 1H), 7.51 (d, J = 8.8 Hz, 1H), 7.44 (d, J = 17.3 Hz, 1H), 6.98 (d, J = 17.3 Hz, 1H), 6.27-6.00 (m, 1H), 4.88-4.83 (m, 1H), 4.53-4.36 (m, 5H), 3.76 (s, 3H), 3.61- 3.51 (m, 4H), 3.26-3.16 (m, 1H), 2.47-2.43 (m, 2H), 2.43 (s, 3H) |
| 75 | 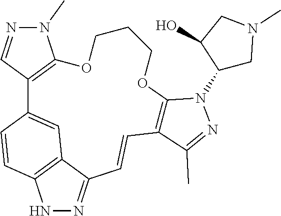 [SMILES: Cc1nn([C@H]2CN(C)C[C@@H]2O)c2c1/C=C/c1n[nH]c3ccc(cc13)-c1cnn(C)c1OCCCO2] | 476.1 | 1H NMR (400 MHz, DMSO- d6) δ = 13.18-12.69 (m, 1H), 8.57 (s, 1H), 7.92 (s, 1H), 7.69 (dd, J = 1.0, 8.8 Hz, 1H), 7.51 (d, J = 8.6 Hz, 1H), 7.44 (d, J = 17.3 Hz, 1H), 6.99 (d, J = 17.3 Hz, 1H), 6.39-6.17 (m, 1H), 4.98-4.81 (m, 1H), 4.44 (br dd, J = 4.4, 11.1 Hz, 5H), 3.76 (s, 3H), 3.51-3.45 (m, 3H), 3.01 (br s, 4H), 2.44 (s, 3H), 2.43-2.40 (m, 2H) |
| 76 | 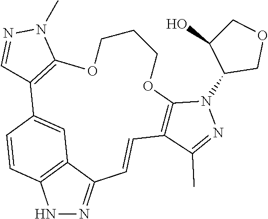 [SMILES: Cc1nn([C@H]2COC[C@@H]2O)c2c1/C=C/c1n[nH]c3ccc(cc13)-c1cnn(C)c1OCCCO2] | 462.8 | 1H NMR (400 MHz, DMSO- d6) δ = 12.95 (s, 1H), 8.57 (s, 1H), 7.91 (s, 1H), 7.72-7.65 (m, 1H), 7.52-7.39 (m, 2H), 6.94 (d, J = 17.2 Hz, 1H), 5.59 (d, J = 4.8 Hz, 1H), 4.70-4.62 (m, 1H), 4.50-4.34 (m, 5H), 4.20 (br d, J = 1.2 Hz, 1H), 4.09-4.02 (m, 1H), 3.86-3.81 (m, 1H), 3.74 (s, 3H), 3.67- 3.62 (m, 1H), 2.39 (s, 5H) |
| 77 | 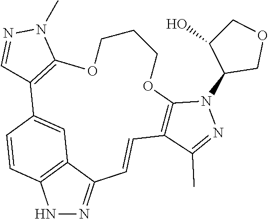 [SMILES: Cc1nn([C@@H]2COC[C@H]2O)c2c1/C=C/c1n[nH]c3ccc(cc13)-c1cnn(C)c1OCCCO2] | 462.9 | 1H NMR (400 MHz, DMSO- d6) δ = 12.94 (br s, 1H), 8.57 (s, 1H), 7.91 (s, 1H), 7.70- 7.66 (m, 1H), 7.52-7.41 (m, 2H), 6.94 (d, J = 17.2 Hz, 1H), 5.59 (d, J = 4.8 Hz, 1H), 4.65 (br s, 1H), 4.46-4.35 (m, 5H), 4.23-4.17 (m, 1H), 4.08-4.01 (m, 1H), 3.86-3.81 (m, 1H), 3.74 (s, 3H), 3.66-3.62 (m, 1H), 2.39 (s, 5H) |
| 78 | 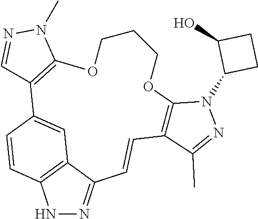 [SMILES: Cc1nn([C@H]2CC[C@@H]2O)c2c1/C=C/c1n[nH]c3ccc(cc13)-c1cnn(C)c1OCCCO2] | 447.3 | 1H NMR (400 MHz, DMSO- d6) δ = 13.30-12.47 (m, 1H), 8.56 (s, 1H), 7.91 (s, 1H), 7.68 (dd, J = 1.2, 8.8 Hz, 1H), 7.50 (d, J = 8.8 Hz, 1H), 7.44 (d, J = 17.2 Hz, 1H), 6.93 (d, J = 17.2 Hz, 1H), 4.83-4.76 (m, 1H), 4.52-4.46 (m, 1H), 4.43-4.37 (m, 2H), 4.31 (br dd, J = 4.8, 9.2 Hz, 2H), 3.74 (s, 3H), 2.42 (s, 3H), 2.37 (br s, 2H), 2.27- 2.09 (m, 4H). |
| 79 | 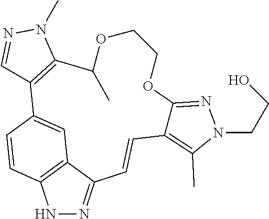 [SMILES: Cc1c2c(nn1CCO)OCCOC(C)c1c(cnn1C)-c1ccc3[nH]nc(c3c1)/C=C/2] | 435.5 | 1H NMR (400 MHz, DMSO- d6) δ = 13.27-12.20 (m, 1H), 8.89-8.61 (m, 1H), 7.62-7.59 (m, 1H), 7.59-7.53 (m, 1H), 7.49 (br d, J = 7.8 Hz, 2H), 6.90 (d, J = 17.6 Hz, 1H), 5.13- 4.96 (m, 1H), 4.30-4.20 (m, 2H), 4.03-4.00 (m, 2H), 3.96 (s, 3H), 3.93-3.91 (m, 2H), 3.69-3.65 (m, 2H), 2.47 (s, 3H), 1.34 (br d, J = 5.6 Hz, 3H) |
| 80 | 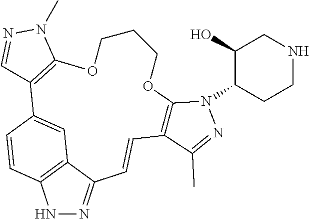 [SMILES: Cc1nn([C@H]2CCNC[C@@H]2O)c2c1/C=C/c1n[nH]c3ccc(cc13)-c1cnn(C)c1OCCCO2] | 476.2 | 1H NMR (400 MHz, METHANOL-d4) δ = 8.65- 8.38 (m, 2H), 7.84-7.78 (m, 1H), 7.64 (br d, J = 8.9 Hz, 1H), 7.53-7.43 (m, 2H), 6.95 (dd, J = 2.4, 17.3 Hz, 1H), 4.50 (dt, J = 5.6, 9.3 Hz, 1H), 4.45- 4.36 (m, 2H), 4.35-4.29 (m, 2H), 4.29-4.18 (m, 1H), 4.12 (dtd, J = 4.6, 9.6, 14.4 Hz, 1H), 3.77-3.72 (m, 3H), 3.55-3.44 (m, 2H), 3.42-3.34 (m, 1H), 3.16-3.06 (m, 1H), 2.97-2.86 (m, 1H), 2.59-2.46 (m, 3H), |
| | | | 2.41 (br s, 1H), 2.37-2.26 (m, |
| | | | 2H), 2.23-2.08 (m, 1H) |
| 81 | 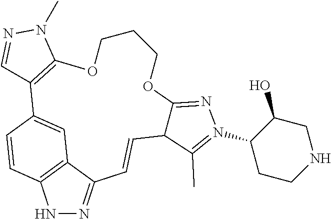 | 476.2 | 1H NMR (400 MHz, METHANOL-d4) δ = 8.66- 8.45 (m, 2H), 7.90-7.77 (m, 1H), 7.65 (br d, J = 8.8 Hz, 1H), 7.55-7.42 (m, 2H), 6.96 (dd, J = 3.6, 17.3 Hz, 1H), 4.55- 4.49 (m, 1H), 4.47-4.37 (m, 2H), 4.36-4.29 (m, 2H), 4.24 (dt, J = 4.3, 10.3 Hz, 1H), 4.16- 4.06 (m, 1H), 3.75 (d, J = 1.9 Hz, 3H), 3.55-3.44 (m, 2H), 3.41-3.33 (m, 1H), 3.15-3.04 (m, 1H), 2.95-2.86 (m, 1H), 2.60-2.47 (m, 3H), 2.43 (br s, |
| | | | 1H), 2.38-2.27 (m, 2H), 2.20- |
| | | | 2.08 (m, 1H) |
| 82 | 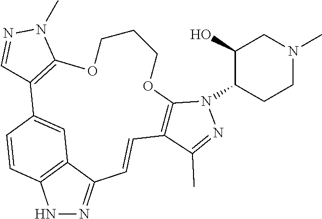 [SMILES: Cc1nn([C@H]2CCN(C)C[C@@H]2O)c2c1/C=C/c1n[nH]c3ccc(cc13)-c1cnn(C)c1OCCCO2] | 490.3 | 1H NMR (400 MHz, METHANOL-d4) δ = 8.63- 8.36 (m, 2H), 7.85-7.73 (m, 1H), 7.66-7.55 (m, 1H), 7.50- 7.36 (m, 2H), 6.96-6.87 (m, 1H), 4.55-4.42 (m, 1H), 4.42- 4.32 (m, 2H), 4.32-4.22 (m, 2H), 4.20-4.07 (m, 1H), 3.74- 3.69 (m, 3H), 3.62-3.43 (m, 2H), 3.42-3.33 (m, 1H), 3.28- 3.11 (m, 1H), 2.97 (br t, J = 11.4 Hz, 1H), 2.63-2.53 (m, 3H), 2.53-2.41 (m, 2H), 2.41- 2.27 (m, 3H), 2.26-2.10 (m, |
| | | | 1H |
| 83 | 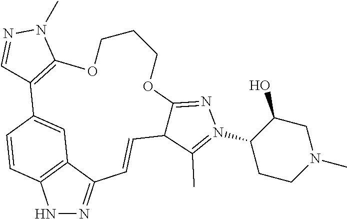 | 490.3 | 1H NMR (400 MHz, METHANOL-d4) δ = 8.58- 8.40 (m, 2H), 7.83-7.73 (m, 1H), 7.65-7.55 (m, 1H), 7.49- 7.38 (m, 2H), 6.96-6.87 (m, 1H), 4.51-4.40 (m, 1H), 4.40- 4.32 (m, 2H), 4.31-4.20 (m, 2H), 4.20-4.08 (m, 1H), 3.75- 3.68 (m, 3H), 3.66-3.38 (m, 2H), 3.34 (br s, 1H), 3.27- 3.12 (m, 1H), 2.97 (br t, J = 11.5 Hz, 1H), 2.62-2.53 (m, 3H), 2.45 (d, J = 7.6 Hz, 2H), 2.39-2.26 (m, 3H), 2.25-2.11 |
| | | | (m, 1H) |
| 84 | 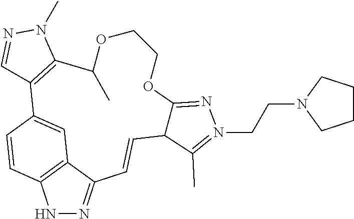 | 488.1 | 1H NMR (400 MHz, DMSO- d6) δ = 13.33-12.36 (m, 1H), 8.89-8.58 (m, 1H), 7.60 (s, 1H), 7.56 (br d, J = 17.8 Hz, 1H), 7.53-7.44 (m, 2H), 6.97 (d, J = 17.6 Hz, 1H), 5.11- 4.97 (m, 1H), 4.36 (br t, J = 6.0 Hz, 2H), 4.26 (br s, 2H), 3.97 (s, 3H), 3.94-3.91 (m, 1H), 3.85 (br s, 1H), 3.59 (br s, 4H), 3.11-2.99 (m, 2H), 2.52 (s, 3H), 2.07-1.94 (m, 2H), 1.92- 1.80 (m, 2H), 1.38-1.31 (m, 3H) |
| 85 | 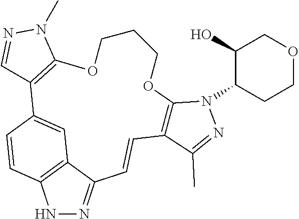 [SMILES: Cc1nn([C@H]2CCOC[C@@H]2O)c2c1/C=C/c1n[nH]c3ccc(cc13)-c1cnn(C)c1OCCCO2] | 477.2 | 1H NMR (400 MHz, METHANOL-d4) δ = 8.53- 8.44 (m, 1H), 7.82 (s, 1H), 7.77 (d, J = 7.8 Hz, 1H), 7.46 (d, J = 8.8 Hz, 1H), 6.83 (d, J = 16.4 Hz, 1H), 6.64-6.56 (m, 1H), 5.73-5.60 (m, 1H), 5.26- 5.18 (m, 2H), 5.10 (br d, J = 10.3 Hz, 1H), 4.12-4.07 (m, 1H), 4.00-3.91 (m, 3H), 3.52- 3.45 (m, 2H), 3.20-3.16 (m, 1H), 2.82 (br d, J = 7.8 Hz, 2H), 2.18-2.15 (m, 3H), 2.14- 2.02 (m, 2H), 1.87-1.80 (m, |
| | | | 1H), 1.30-1.27 (m, 1H) |
| 86 | 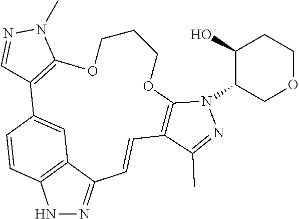 [SMILES: Cc1nn([C@H]2COCC[C@@H]2O)c2c1/C=C/c1n[nH]c3ccc(cc13)-c1cnn(C)c1OCCCO2] | 477.6 | 1H NMR (400 MHz, METHANOL-d4) δ = 8.52- 8.43 (m, 1H), 7.86 (s, 1H), 7.76 (d, J = 8.4 Hz, 1H), 7.47 (d, J = 8.8 Hz, 1H), 6.83 (d, J = 16.5 Hz, 1H), 6.65- 6.54 (m, 1H), 5.74-5.62 (m, 1H), 5.30-5.15 (m, 2H), 5.10 (br d, J = 10.5 Hz, 1H), 4.13- 4.05 (m, 1H), 4.03-3.88 (m, 3H), 3.54-3.36 (m, 2H), 3.19 (t, J = 10.6 Hz, 1H), 2.82 (br d, J = 8.0 Hz, 2H), 2.19-2.14 (m, 3H), 2.04 (s, 2H), 1.88-1.80 |
| | | | (m, 1H), 1.32-1.26 (m, 1H) |
| 87 | 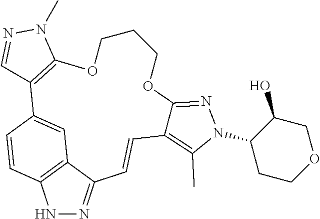 [SMILES: Cc1c2c(nn1[C@H]1CCOC[C@@H]1O)OCCCOc1c(cnn1C)-c1ccc3[nH]nc(c3c1)/C=C/2] | 477.1 | 1H NMR (400 MHz, METHANOL-d4) δ = 8.53- 8.40 (m, 1H), 7.83-7.72 (m, 2H), 7.49-7.41 (m, 1H), 6.94 (br d, J = 17.4 Hz, 1H), 6.59 (br d, J = 15.6 Hz, 1H), 5.51- 5.48 (m, 1H), 5.20 (br d, J = 4.4 Hz, 2H), 5.09 (br s, 1H), 4.14-4.07 (m, 1H), 4.04-3.90 (m, 3H), 3.52-3.47 (m, 2H), 3.21-3.13 (m, 1H), 2.93-2.74 (m, 2H), 2.17 (br d, J = 2.1 Hz, 2H), 2.04 (d, J = 2.3 Hz, 3H), 1.78-1.71 (m, 1H), 1.30-1.28 |
| | | | (m, 1H) |
| 88 | 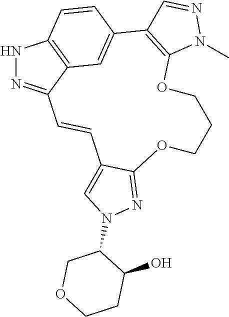 [SMILES: Cn1ncc2c1OCCCOc1nn([C@H]3COCC[C@@H]3O)cc1/C=C/c1n[nH]c3ccc-2cc13] | 476.9 | 1H NMR (400 MHz, METHANOL-d4) δ = 8.54 (br d, J = 2.9 Hz, 1H), 7.70-7.67 (m, 1H), 7.53-7.50 (m, 1H), 7.47-7.43 (m, 1H), 6.86-6.79 (m, 1H), 6.68-6.62 (m, 1H), 5.37-5.31 (m, 1H), 5.18 (br d, J = 7.9 Hz, 3H), 4.17-4.13 (m, 1H), 4.11-4.01 (m, 3H), 3.81-3.77 (m, 3H), 3.02 (br d, J = 7.3 Hz, 2H), 2.42-2.39 (m, 1H), 2.19 (br t, J = 7.9 Hz, 2H), 2.08-1.99 (m, 3H), 1.27 (br s, 1H) |
| 89 | 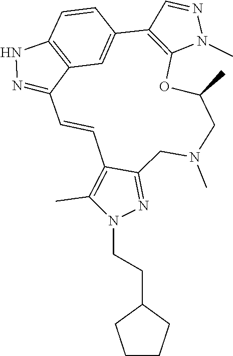 [SMILES: Cc1c2c(nn1CCC1CCCC1)CN(C)C[C@H](C)Oc1c(cnn1C)-c1ccc3[nH]nc(c3c1)/C=C/2] | 501.1 | 1H NMR (400 MHz, DMSO- d6) δ = 13.21-12.83 (m, 1H), 10.24-9.96 (m, 1H), 8.48 (s, 1H), 7.90 (br d, J = 1.4 Hz, 1H), 7.69 (br s, 1H), 7.54 (br d, J = 8.0 Hz, 1H), 7.52-7.40 (m, 1H), 7.10-6.96 (m, 1H), 5.51-5.00 (m, 1H), 4.92-4.67 (m, 1H), 4.63-4.40 (m, 3H), 4.07-3.84 (m, 2H), 3.73 (br s, 3H), 3.69-3.60 (m, 3H), 3.58 (br d, J = 1.3 Hz, 2H), 3.13- 2.88 (m, 4H), 2.59 (br s, 2H), 2.03 (br s, 2H), 1.86 (br d, J = 5.8 Hz, 2H), 1.05 (br s, 3H) |
| 90 | 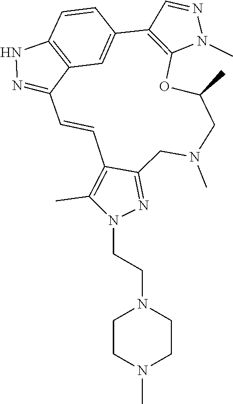 [SMILES: Cc1c2c(nn1CCN1CCN(C)CC1)CN(C)C[C@H](C)Oc1c(cnn1C)-c1ccc3[nH]nc(c3c1)/C=C/2] | 530.1 | 1H NMR (600 MHz, DMSO- d6) δ = 13.01-12.78 (m, 1H), 9.54-9.35 (m, 1H), 8.48 (br s, 1H), 8.19 (br s, 1H), 7.86 (s, 1H), 7.68 (br d, J = 8.5 Hz, 1H), 7.50 (br d, J = 8.2 Hz, 1H), 6.91 (br d, J = 17.3 Hz, 1H), 4.88-4.71 (m, 1H), 4.17 (br d, J = 1.8 Hz, 1H), 3.90 (br d, J = 12.6 Hz, 1H), 3.68 (br s, 3H), 3.04-2.96 (m, 4H), 2.81- 2.78 (m, 4H), 2.62 (br s, 1H), 2.57-2.55 (m, 8H), 2.39 (br s, 1H), 2.35 (br s, 1H), 2.26 (br s, 3H), 2.05-1.96 (m, 1H), 1.02 (br d, J = 4.7 Hz, 3H) |
| 91 | 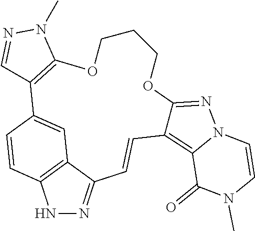 [SMILES: Cn1ncc2c1OCCCOc1nn3ccn(C)c(=O)c3c1/C=C/c1n[nH]c3ccc-2cc13] | 443.9 | 1H NMR (400 MHz, DMSO- d6) δ = 13.02 (s, 1H), 8.51 (s, 1H), 8.31 (d, J = 16.8 Hz, 1H), 7.90 (s, 1H), 7.73-7.67 (m, 1H), 7.63 (d, J = 5.9 Hz, 1H), 7.53 (s, 1H), 7.46 (s, 1H), 7.16 (d, J = 5.9 Hz, 1H), 4.45 (t, J = 5.4 Hz, 4H), 3.73 (s, 3H), 3.47 (s, 3H), 2.42-2.34 (m, 2H) |
| 92 | 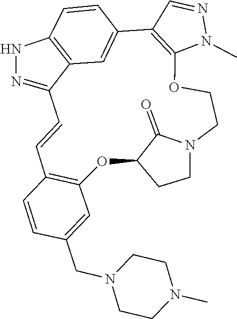 [SMILES: CN1CCN(Cc2ccc3c(c2)O[C@@H]2CCN(CCOc4c(cnn4C)-c4ccc5[nH]nc(c5c4)/C=C/3)C2=O)CC1] | 554.3 | 1H NMR (400 MHz, METHANOL-d4) δ = 8.15 (s, 1H), 7.70-7.68 (m, 2H), 7.68- 7.65 (m, 1H), 7.64-7.60 (m, 1H), 7.59-7.57 (m, 1H), 7.57- 7.53 (m, 1H), 7.25 (s, 1H), 7.13 (d, J = 7.5 Hz, 1H), 5.06 (br d, J = 5.6 Hz, 1H), 4.48- 4.39 (m, 1H), 4.35-4.24 (m, 1H), 4.15-4.01 (m, 2H), 3.94 (s, 2H), 3.84 (s, 3H), 3.65- 3.55 (m, 1H), 3.50-3.37 (m, 5H), 3.18-2.97 (m, 4H), 2.91 (s, 3H), 2.48-2.35 (m, 1H), 2.22 (br dd, J = 6.9, 14.3 Hz, 1H) |
| 93 | 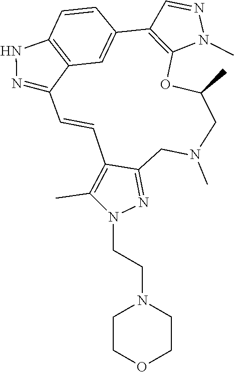 [SMILES: Cc1c2c(nn1CCN1CCOCC1)CN(C)C[C@H](C)Oc1c(cnn1C)-c1ccc3[nH]nc(c3c1)/C=C/2] | 517.0 | 1H NMR (499 MHz, DMSO- d6) δ = 13.13-12.96 (m, 1H), 10.22-10.06 (m, 1H), 8.48 (s, 1H), 8.28-8.15 (m, 1H), 7.98- 7.85 (m, 1H), 7.69 (br d, J = 3.3 Hz, 1H), 7.59-7.48 (m, 1H), 7.04-6.91 (m, 1H), 5.55- 5.41 (m, 1H), 5.14-5.00 (m, 1H), 4.89-4.75 (m, 1H), 4.63- 4.44 (m, 2H), 4.28-4.19 (m, 1H), 4.06-3.85 (m, 4H), 3.68 (br d, J = 4.4 Hz, 4H), 3.24- 3.13 (m, 2H), 3.08 (br dd, J = 5.2, 7.4 Hz, 1H), 2.98 (br s, 1H), 2.93-2.86 (m, 1H), 2.75 (d, J = 4.9 Hz, 1H), 2.66-2.54 (m, 3H), 2.39-2.34 (m, 1H), 2.27 (br d, J = 2.7 Hz, 1H), 2.04-1.95 (m, 1H), 1.18 (br t, J = 7.4 Hz, 3H) |
| 94 | 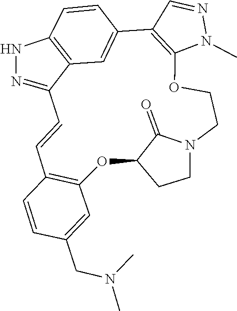 [SMILES: CN(C)Cc1ccc2c(c1)O[C@@H]1CCN(CCOc3c(cnn3C)-c3ccc4[nH]nc(c4c3)/C=C/2)C1=O] | 499.2 | 1H NMR (400 MHz, METHANOL-d4) δ = 8.18 (s, 1H), 7.81 (d, J = 7.9 Hz, 1H), 7.72-7.68 (m, 2H), 7.68-7.65 (m, 1H), 7.64-7.62 (m, 1H), 7.61-7.57 (m, 1H), 7.35-7.32 (m, 1H), 7.22 (d, J = 7.4 Hz, 1H), 5.11-5.07 (m, 1H), 4.50- 4.44 (m, 1H), 4.35-4.28 (m, 3H), 4.18-4.10 (m, 1H), 4.09- 4.03 (m, 1H), 3.86 (s, 3H), 3.64 (br t, J = 8.6 Hz, 1H), 3.52-3.46 (m, 1H), 2.51-2.41 (m, 1H), 2.33-2.22 (m, 1H), 2.04 (s, 6H) |
| 95 | 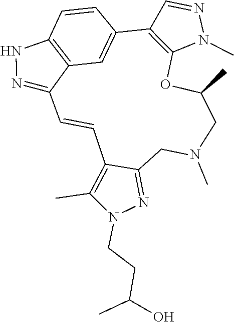 [SMILES: Cc1c2c(nn1CCC(C)O)CN(C)C[C@H](C)Oc1c(cnn1C)-c1ccc3[nH]nc(c3c1)/C=C/2] | 476.3 | 1H NMR (400 MHz, DMSO- d6) δ = 13.08-12.66 (m, 1H), 8.46 (s, 1H), 8.29-8.14 (m, 1H), 7.88-7.82 (m, 1H), 7.70- 7.62 (m, 1H), 7.48 (d, J = 8 Hz, 1H), 6.96-6.83 (m, 1H), 4.85-4.69 (m, 1H), 4.32-4.12 (m, 3H), 3.56 (br s, 1H), 3.53 (br s, 1H), 2.54-2.52 (m, 3H), 1.04-0.98 (m, 3H) |
| 96 | 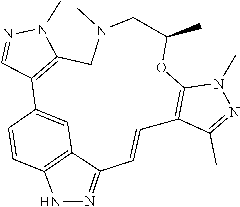 [SMILES: Cc1nn(C)c2c1/C=C/c1n[nH]c3ccc(cc13)-c1cnn(C)c1CN(C)C[C@@H](C)O2] | 418.0 | 1H NMR (400 MHz, METHANOL-d4) δ = 9.13- 9.09 (m, 1H), 7.83-7.75 (m, 1H), 7.72 (s, 1H), 7.59-7.50 (m, 2H), 7.05-6.97 (m, 1H), 4.57-4.49 (m, 1H), 3.99 (s, 3H), 3.97-3.94 (m, 1H), 3.83- 3.75 (m, 1H), 3.64 (s, 3H), 3.06-2.97 (m, 1H), 2.95-2.86 (m, 1H), 2.48 (s, 3H), 2.45- 2.40 (m, 3H), 1.23 (d, J = 6.4 Hz, 3H) |
| 97 | 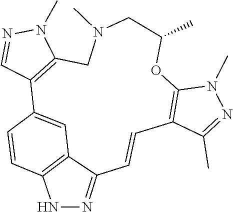 [SMILES: Cc1nn(C)c2c1/C=C/c1n[nH]c3ccc(cc13)-c1cnn(C)c1CN(C)C[C@H](C)O2] | 417.23 | 1H NMR (400 MHz, METHANOL-d4) δ = 9.13- 9.05 (m, 1H), 7.82-7.73 (m, 1H), 7.72-7.68 (m, 1H), 7.59- 7.47 (m, 2H), 7.05-6.93 (m, 1H), 4.59-4.51 (m, 1H), 3.99- 3.95 (m, 3H), 3.94-3.82 (m, 1H), 3.82-3.72 (m, 1H), 3.66- 3.59 (m, 3H), 3.06-2.95 (m, 1H), 2.94-2.82 (m, 1H), 2.49- 2.44 (m, 3H), 2.44-2.39 (m, 3H), 1.21 (br d, J = 6.0 Hz, 3H) |
| 98 | 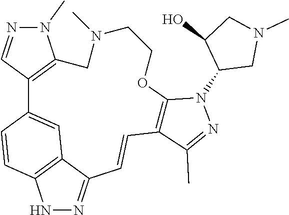 [SMILES: Cc1nn([C@H]2CN(C)C[C@@H]2O)c2c1/C=C/c1n[nH]c3ccc(cc13)-c1cnn(C)c1CN(C)CCO2] | 489.4 | 1H NMR (400 MHz, METHANOL-d4) δ = 7.88 (br d, J = 1.6 Hz, 1H), 7.19 (s, 1H), 6.92 (dd, J = 1.6, 8.8 Hz, 1H), 6.82 (d, J = 8.8 Hz, 1H), 6.78-6.67 (m, 1H), 6.64-6.56 (m, 1H), 4.30-4.26 (m, 2H), 3.86 (br s, 3H), 3.72-3.55 (m, 4H), 3.50-3.33 (m, 2H), 3.06- 2.86 (m, 3H), 2.81-2.72 (m, 2H), 2.51-2.37 (m, 4H), 1.97 (br s, 3H), 1.78 (s, 3H) |
| 99 | 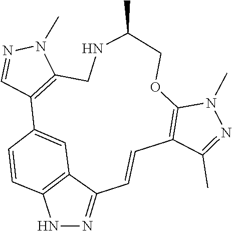 [SMILES: Cc1nn(C)c2c1/C=C/c1n[nH]c3ccc(cc13)-c1cnn(C)c1CN[C@@H](C)CO2] | 403.9 | 1H NMR (400 MHz, DMSO- d6) δ = 13.14-12.93 (m, 1H), 8.34-8.14 (m, 1H), 7.89 (s, 1H), 7.59-7.50 (m, 3H), 6.94 (d, J = 16 Hz, 1H), 4.84-4.64 (m, 1H), 4.58-4.37 (m, 3H), 4.02 (s, 5H), 3.66 (s, 3H), 2.37 (s, 3H), 1.49-1.37 (m, 3H) |
| 100 | 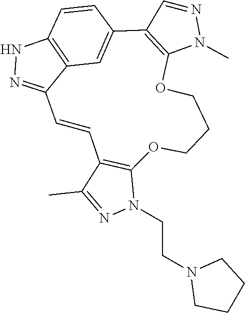 [SMILES: Cc1nn(CCN2CCCC2)c2c1/C=C/c1n[nH]c3ccc(cc13)-c1cnn(C)c1OCCCO2] | 488.2 | 1H NMR (400 MHz, DMSO- d6) δ = 12.91 (s, 1H), 8.92 (br s, 1H), 7.62 (s, 1H), 7.58 (s, 1H), 7.49 (d, J = 2.8 Hz, 2H), 6.92-6.86 (m, 1H), 5.06-4.99 (m, 1H), 4.40-4.32 (m, 1H), 4.26 (br t, J = 9.2 Hz, 1H), 4.13-4.04 (m, 2H), 4.01 (br d, J = 4.8 Hz, 2H), 3.99-3.91 (m, 5H), 2.78-2.73 (m, 2H), 2.46 (br d, J = 4.8 Hz, 2H), 2.36 (s, 3H), 1.66 (br s, 4H), 1.34-1.30 (m, 3H) |
| 101 | 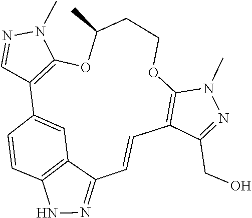 [SMILES: C[C@H]1CCOc2c(c(CO)nn2C)/C=C/c2n[nH]c3ccc(cc23)-c2cnn(C)c2O1] | 421.0 | 1H NMR (400 MHz, DMSO- d6) δ = 8.56 (s, 1H), 7.93 (s, 1H), 7.71 (d, J = 8.7 Hz, 1H), 7.49 (d, J = 8.7 Hz, 1H), 7.41- 7.33 (m, 1H), 7.24-7.16 (m, 1H), 5.20 (br s, 1H), 4.95- 4.85 (m, 1H), 4.63-4.55 (m, 1H), 4.51 (br d, J = 3.4 Hz, 2H), 4.37-4.30 (m, 1H), 3.70 (s, 3H), 3.68 (s, 3H), 2.40 (br s, 2H), 1.31 (d, J = 6.2 Hz, 3H) |
| 102 | 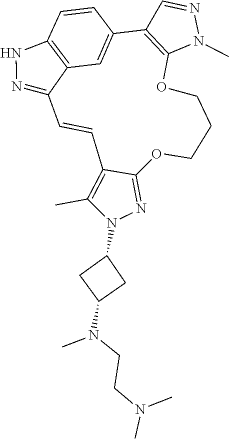 [SMILES: Cc1c2c(nn1[C@H]1C[C@@H](N(C)CCN(C)C)C1)OCCCOc1c(cnn1C)-c1ccc3[nH]nc(c3c1)/C=C/2] | 531.1 | 1H NMR (400 MHz, METHANOL-d4) δ = 8.60 (s, 1H), 7.86 (s, 1H), 7.72-7.67 (m, 1H), 7.57-7.49 (m, 2H), 6.99 (d, J = 17.2 Hz, 1H), 4.74- 4.66 (m, 2H), 4.49 (br t, J = 5.6 Hz, 2H), 4.44-4.37 (m, 2H), 3.79 (s, 3H), 3.04 (br t, J = 6.0 Hz, 2H), 2.98-2.93 (m, 6H), 2.88 (d, J = 2.0 Hz, 1H), 2.81 (br d, J = 5.6 Hz, 3H), 2.67 (s, 1H), 2.65-2.57 (m, 2H), 2.54 (s, 5H), 2.43-2.38 (m, 2H) |
| 103 | 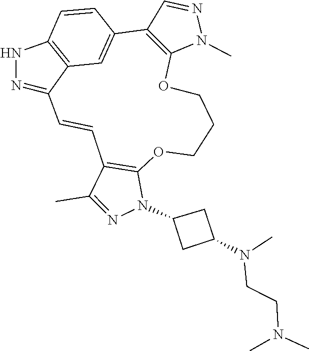 [SMILES: Cc1nn([C@H]2C[C@@H](N(C)CCN(C)C)C2)c2c1/C=C/c1n[nH]c3ccc(cc13)-c1cnn(C)c1OCCCO2] | 531.1 | 1H NMR (400 MHz, METHANOL-d4) δ = 8.68 (s, 1H), 7.88 (s, 1H), 7.73-7.68 (m, 1H), 7.58-7.50 (m, 2H), 7.03 (d, J = 17.2 Hz, 1H), 4.69- 4.63 (m, 2H), 4.51 (br t, J = 5.2 Hz, 2H), 4.40 (br t, J = 4.8 Hz, 2H), 3.80 (s, 3H), 3.09- 3.00 (m, 1H), 2.94 (s, 6H), 2.86-2.79 (m, 2H), 2.79-2.67 (m, 3H), 2.58-2.53 (m, 1H), 2.51 (s, 4H), 2.49 (br d, J = 4.0 Hz, 2H), 2.39 (s, 3H) |
| 104 | 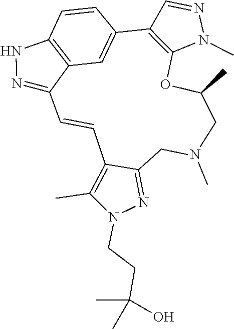 [SMILES: Cc1c2c(nn1CCC(C)(C)O)CN(C)C[C@H](C)Oc1c(cnn1C)-c1ccc3[nH]nc(c3c1)/C=C/2] | 490.5 | 1H NMR (400 MHz, DMSO- d6) δ = 12.95-12.83 (m, 1H), 8.49-8.44 (m, 1H), 8.23-8.14 (m, 1H), 7.85 (s, 1H), 7.68- 7.64 (m, 1H), 7.51-7.46 (m, 1H), 6.92-6.84 (m, 1H), 4.83- 4.72 (m, 1H), 4.29-4.06 (m, 2H), 3.66 (s, 3H), 3.59 (s, 1H), 3.53 (s, 2H), 2.94-2.87 (m, 2H), 2.78-2.68 (m, 2H), 2.54 (br s, 3H), 2.26-2.22 (m, 3H), 2.04-1.94 (m, 2H), 1.23 (br s, 3H), 1.14 (d, J = 2 Hz, 3H), 1.03-0.98 (m, 3H) |
| 105 | 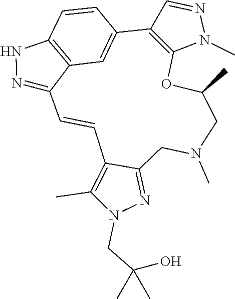 [SMILES: Cc1c2c(nn1CC(C)(C)O)CN(C)C[C@H](C)Oc1c(cnn1C)-c1ccc3[nH]nc(c3c1)/C=C/2] | 476.3 | 1H NMR (400 MHz, DMSO- d6) δ = 12.95-12.86 (m, 1H), 8.48 (s, 1H), 8.24-8.15 (m, 1H), 7.87-7.84 (m, 1H), 7.67 (br d, J = 8.8 Hz, 1H), 7.52- 7.47 (m, 1H), 6.97-6.85 (m, 1H), 4.79 (br t, J = 6.0 Hz, 1H), 4.70 (s, 1H), 3.98 (s, 2H), 3.91 (br d, J = 12.9 Hz, 1H), 3.72 (s, 1H), 3.67 (s, 3H), 2.90 (br d, J = 13.3 Hz, 2H), 2.52 (br s, 3H), 2.26 (s, 3H), 1.12 (d, J = 5.0 Hz, 6H), 1.02 (d, J = 6.3 Hz, 3H) |
| 106 | 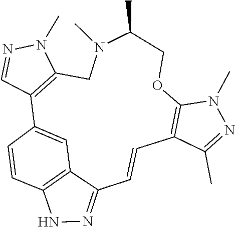 [SMILES: Cc1nn(C)c2c1/C=C/c1n[nH]c3ccc(cc13)-c1cnn(C)c1CN(C)[C@@H](C)CO2] | 418.2 | 1H NMR (400 MHz, DMSO- d6) δ = 12.96 (s, 1H), 8.59 (s, 1H), 7.83 (s, 1H), 7.61-7.56 (m, 1H), 7.55-7.50 (m, 1H), 7.38 (d, J = 17.2 Hz, 1H), 6.94 (d, J = 17.2 Hz, 1H), 4.39- 4.30 (m, 2H), 3.91 (s, 4H), 3.79 (d, J = 13.2 Hz, 1H), 3.58 (s, 3H), 3.54-3.48 (m, 1H), 2.33 (s, 3H), 2.18 (s, 3H), 1.28 (d, J = 6.4 Hz, 3H) |
| 107 | 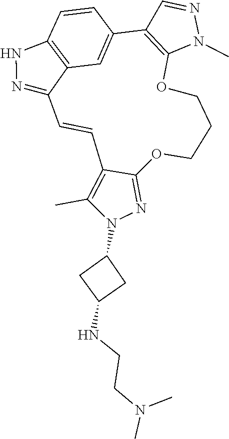 [SMILES: Cc1c2c(nn1[C@H]1C[C@@H](NCCN(C)C)C1)OCCCOc1c(cnn1C)-c1ccc3[nH]nc(c3c1)/C=C/2] | 517.0 | 1H NMR (400 MHz, METHANOL-d4) δ = 8.58 (s, 1H), 7.86 (s, 1H), 7.70 (dd, J = 1.2, 8.8 Hz, 1H), 7.56-7.48 (m, 2H), 6.98 (d, J = 17.6 Hz, 1H), 4.49 (t, J = 5.6 Hz, 2H), 4.41 (t, J = 5.2 Hz, 2H), 3.78 (s, 3H), 3.50-3.47 (m, 1H), 3.44-3.38 (m, 4H), 3.07-3.01 (m, 1H), 2.97 (s, 2H), 2.94 (s, 5H), 2.84-2.74 (m, 2H), 2.54 (s, 2H), 2.50 (s, 1H), 2.43- 2.36 (m, 2H), 2.04 (s, 2H) |
| 108 | 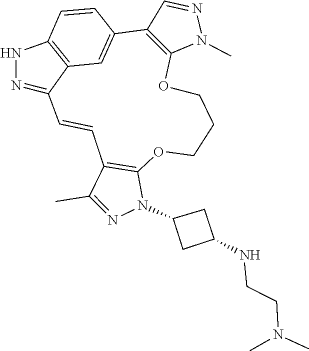 [SMILES: Cc1nn([C@H]2C[C@@H](NCCN(C)C)C2)c2c1/C=C/c1n[nH]c3ccc(cc13)-c1cnn(C)c1OCCCO2] | 517.0 | 1H NMR (400 MHz, METHANOL-d4) δ = 8.50 (br s, 1H), 7.81-7.75 (m, 1H), 7.60 (br d, J = 8.8 Hz, 1H), 7.46-7.41 (m, 1H), 7.37 (br d, J = 17.2 Hz, 1H), 6.89 (br d, J = 17.2 Hz, 1H), 4.77-4.66 (m, 2H), 4.30 (br d, J = 4.4 Hz, 2H), 4.16 (br d, J = 4.4 Hz, 2H), 3.83-3.75 (m, 1H), 3.68 (s, 3H), 3.61-3.44 (m, 4H), 3.01-2.98 (m, 6H), 2.97-2.88 (m, 2H), 2.86-2.73 (m, 2H), 2.46-2.40 (m, 3H), 2.28 (br d, J = 4.0 Hz, 2H) |
| 109 | 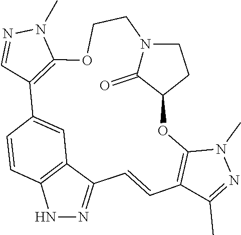 | 460.1 | 1H NMR (400 MHz, METHANOL-d4) δ = 8.06 (s, 1H), 7.74-7.67 (m, 1H), 7.63- 7.59 (m, 1H), 7.57-7.52 (m, 1H), 7.30-7.24 (m, 1H), 7.20 (s, 1H), 4.97 (br dd, J = 4.0, 7.4 Hz, 1H), 4.45-4.35 (m, 1H), 4.23 (ddd, J = 2.1, 6.6, 14.6 Hz, 1H), 4.15-4.05 (m, 1H), 3.98-3.90 (m, 1H), 3.83 (s, 3H), 3.77 (s, 3H), 3.57- 3.47 (m, 1H), 3.43-3.36 (m, 1H), 2.52-2.42 (m, 2H), 2.39 (s, 3H) |
| 110 | 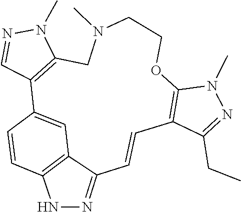 [SMILES: CCc1nn(C)c2c1/C=C/c1n[nH]c3ccc(cc13)-c1cnn(C)c1CN(C)CCO2] | 418.4 | 1H NMR (400 MHz, DMSO- d6) δ = 13.17-12.78 (m, 1H), 8.94-8.46 (m, 1H), 8.04-7.76 (m, 1H), 7.61 (br d, J = 7.9 Hz, 1H), 7.57-7.51 (m, 1H), 7.47 (d, J = 17.4 Hz, 1H), 6.91 (d, J = 17.3 Hz, 1H), 4.58-4.32 (m, 2H), 4.03-3.90 (m, 3H), 3.77- 3.68 (m, 2H), 3.65 (br s, 2H), 3.06-2.90 (m, 2H), 2.77 (q, J = 7.3 Hz, 2H), 2.53-2.51 (m, 3H), 1.26 (t, J = 7.4 Hz, 3H) |
| 111 | 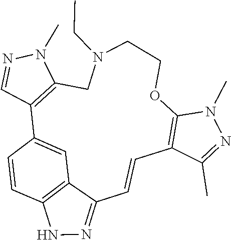 [SMILES: CCN1CCOc2c(c(C)nn2C)/C=C/c2n[nH]c3ccc(cc23)-c2cnn(C)c2C1] | 418.2 | 1H NMR (400 MHz, METHANOL-d4) δ = 8.56 (br s, 1H), 7.95 (s, 1H), 7.62 (br d, J = 6.4 Hz, 3H), 7.05 (d, J = 17.3 Hz, 1H), 4.58 (br s, 4H), 4.06 (s, 3H), 3.71 (s, 5H), 3.29- 3.19 (m, 2H), 2.44 (s, 3H), 1.32 (br t, J = 6.8 Hz, 3H) |
| 112 | 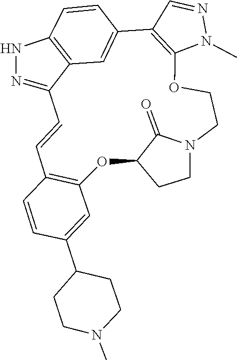 [SMILES: CN1CCC(c2ccc3c(c2)O[C@@H]2CCN(CCOc4c(cnn4C)-c4ccc5[nH]nc(c5c4)/C=C/3)C2=O)CC1] | 539.2 | 1H NMR (400 MHz, METHANOL-d4) δ = 8.18 (s, 1H), 7.68 (s, 3H), 7.61 (d, J = 1.2 Hz, 1H), 7.56 (s, 1H), 7.55- 7.50 (m, 1H), 7.09 (s, 1H), 7.03 (dd, J = 1.3, 8.1 Hz, 1H), 5.10 (s, 1H), 4.40-4.32 (m, 2H), 4.12-4.05 (m, 2H), 3.85 (s, 3H), 3.62-3.51 (m, 2H), 3.49-3.43 (m, 2H), 2.98-2.82 (m, 3H), 2.78 (s, 3H), 2.41- 2.32 (m, 1H), 2.20 (br dd, J = 6.8, 14.2 Hz, 1H), 2.10 (br d, J = 12.3 Hz, 2H), 2.02-1.88 (m, 2H) |
| 113 | 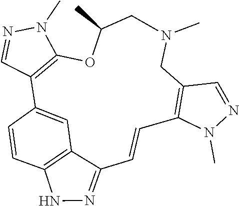 [SMILES: C[C@H]1CN(C)Cc2cnn(C)c2/C=C/c2n[nH]c3ccc(cc23)-c2cnn(C)c2O1] | 404.2 | 1H NMR (400 MHz, METHANOL-d4) δ = 8.58 (s, 1H), 7.88 (s, 1H), 7.77-7.67 (m, 3H), 7.62 (d, J = 8.0 Hz, 1H), 7.45 (d, J = 16.0 Hz, 1H), 5.54-5.37 (m, 1H), 4.20 (s, 3H), 4.15-4.00 (m, 1H), 3.92- 3.80 (m, 2H), 3.79 (s, 3H), 3.64 (br dd, J = 2.0, 14.6 Hz, 1H), 3.05 (br s, 3H), 1.18 (d, J = 6.0 Hz, 3H) |
| 114 | 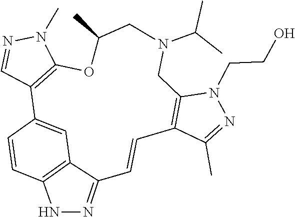 | 476.3 | 1H NMR (400 MHz, DMSO- d6) δ = 12.86 (s, 1H), 8.64- 8.59 (m, 1H), 8.04 (br d, J = 17.4 Hz, 1H), 7.87 (s, 1H), 7.67-7.63 (m, 1H), 7.47 (d, J = 8.6 Hz, 1H), 6.92 (d, J = 17.4 Hz, 1H), 4.94-4.61 (m, 2H), 4.12 (t, J = 5.6 Hz, 2H), 4.01- 3.90 (m, 2H), 3.69 (s, 5H), 2.91-2.74 (m, 2H), 2.71-2.64 (m, 1H), 2.41 (s, 3H), 1.06 (br d, J = 6.4 Hz, 3H), 0.93 (br d, J = 6.4 Hz, 6H) |
| 115 | 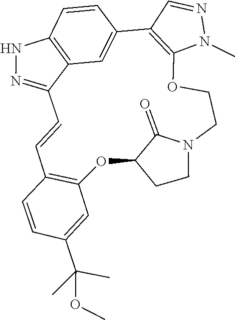 [SMILES: COC(C)(C)c1ccc2c(c1)O[C@@H]1CCN(CCOc3c(cnn3C)-c3ccc4[nH]nc(c4c3)/C=C/2)C1=O] | 514.2 | 1H NMR (400 MHz, METHANOL-d4) δ = 8.18 (s, 1H), 7.75-7.69 (m, 1H), 7.69- 7.64 (m, 2H), 7.63-7.59 (m, 1H), 7.55 (br d, J = 4.0 Hz, 1H), 7.22 (d, J = 1.6 Hz, 1H), 7.15 (dd, J = 1.5, 8.1 Hz, 1H), 5.07 (br d, J = 6.0 Hz, 1H), 4.44-4.37 (m, 1H), 4.36-4.29 (m, 1H), 4.11-4.05 (m, 2H), 3.87-3.83 (m, 3H), 3.62-3.53 (m, 1H), 3.43 (td, J = 6.2, 8.2 Hz, 1H), 3.13 (s, 3H), 2.46- 2.35 (m, 1H), 2.22 (br dd, J = 7.0, 14.4 Hz, 1H), 1.59-1.52 (m, 6H) |
| 116 | 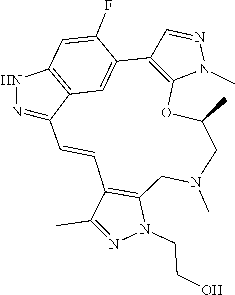 [SMILES: Cc1nn(CCO)c2c1/C=C/c1n[nH]c3cc(F)c(cc13)-c1cnn(C)c1O[C@@H](C)CN(C)C2] | 466.3 | 1H NMR (400 MHz, DMSO- d6) δ = 8.59 (d, J = 7.4 Hz, 1H), 8.10-7.96 (m, 1H), 7.75 (d, J = 5.1 Hz, 1H), 7.34 (d, J = 12.0 Hz, 1H), 6.94 (d, J = 17.4 Hz, 1H), 4.90-4.80 (m, 1H), 4.20-4.12 (m, 3H), 4.11-4.04 (m, 1H), 3.77 (br s, 1H), 3.76- 3.74 (m, 2H), 3.74 (s, 3H), 3.16-3.06 (m, 2H), 2.44 (s, 6H), 1.07 (d, J = 6.4 Hz, 3H) |
| 117 | 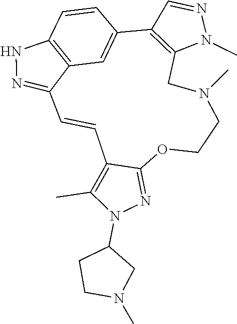 [SMILES: Cc1c2c(nn1C1CCN(C)C1)OCCN(C)Cc1c(cnn1C)-c1ccc3[nH]nc(c3c1)/C=C/2] | 473.1 | 1H NMR (400 MHz, METHANOL-d4) δ = 8.98 (s, 1H), 8.53 (s, 1H), 7.84-7.72 (m, 2H), 7.60 (d, J = 8.4 Hz, 1H), 7.48 (d, J = 8.4 Hz, 1H), 7.20-7.12 (m, 1H), 4.42 (d, J = 2.4 Hz, 2H), 3.95 (d, J = 1.6 Hz, 3H), 3.73 (d, J = 4.0 Hz, 2H), 3.66-3.50 (m, 3H), 3.38- 3.31 (m, 2H), 2.96 (s, 2H), 2.92 (s, 3H), 2.61-2.49 (m, 1H), 2.40 (s, 3H), 2.34-2.23 (m, 4H) |
| 118 | 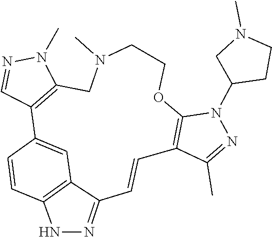 [SMILES: Cc1nn(C2CCN(C)C2)c2c1/C=C/c1n[nH]c3ccc(cc13)-c1cnn(C)c1CN(C)CCO2] | 473.1 | 1H NMR (400 MHz, METHANOL-d4) δ = 8.91 (s, 1H), 7.85 (s, 1H), 7.66-7.62 (m, 1H), 7.58-7.54 (m, 1H), 7.52-7.46 (m, 1H), 7.38-7.35 (m, 1H), 7.09 (br d, J = 17.2 Hz, 1H), 4.50 (br s, 4H), 3.99 (s, 3H), 3.79 (s, 2H), 3.71- 3.51 (m, 3H), 3.11 (br s, 2H), 2.93 (br s, 2H), 2.70-2.58 (m, 1H), 2.50 (br s, 3H), 2.38 (s, 3H), 2.35-2.23 (m, 1H), 2.05 (s, 2H) |
| 119 | 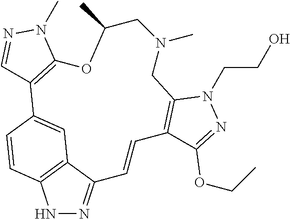 [SMILES: CCOc1nn(CCO)c2c1/C=C/c1n[nH]c3ccc(cc13)-c1cnn(C)c1O[C@@H](C)CN(C)C2] | 478.1 | 1H NMR (400 MHz, METHANOL-d4) δ = 8.55 (s, 1H), 8.29-8.18 (m, 1H), 7.83 (s, 1H), 7.70-7.66 (m, 1H), 7.49 (d, J = 8.8 Hz, 1H), 7.31 (d, J = 16.8 Hz, 1H), 4.35 (dd, J = 2.6, 6.8 Hz, 2H), 4.18- 4.05 (m, 4H), 3.95 (br d, J = 11.8 Hz, 1H), 3.86-3.82 (m, 2H), 3.74 (s, 3H), 3.06-2.95 (m, 2H), 2.51 (br s, 3H), 1.49 (t, J = 7.0 Hz, 3H), 1.13 (d, J = 6.4 Hz, 3H) |
| 120 | 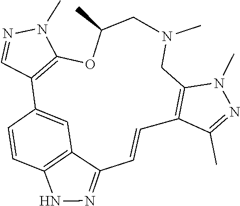 [SMILES: Cc1nn(C)c2c1/C=C/c1n[nH]c3ccc(cc13)-c1cnn(C)c1O[C@@H](C)CN(C)C2] | 418.1 | 1H NMR (400 MHz, METHANOL-d4) δ = 8.54 (s, 1H), 8.35 (d, J = 17.6 Hz, 1H), 7.82 (s, 1H), 7.68 (d, J = 8.8 Hz, 1H), 7.50 (d, J = 8.8 Hz, 1H), 6.99 (d, J = 17.6 Hz, 1H), 4.93-4.91 (m, 1H), 4.01-3.93 (m, 1H), 3.87-3.81 (m, 1H), 3.80 (s, 3H), 3.74 (s, 3H), 2.95- 2.87 (m, 2H), 2.47 (s, 3H), 2.38 (s, 3H), 1.09 (d, J = 6.0 Hz, 3H) |
| 121 | 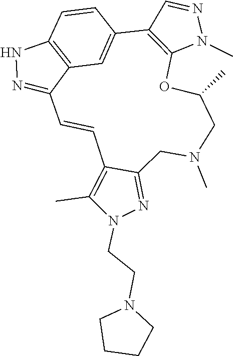 [SMILES: Cc1c2c(nn1CCN1CCCC1)CN(C)C[C@@H](C)Oc1c(cnn1C)-c1ccc3[nH]nc(c3c1)/C=C/2] | 501.3 | 1H NMR (400 MHz, DMSO- d6) δ = 13.12-12.61 (m, 1H), 8.47 (s, 1H), 8.23-8.15 (m, 2H), 7.85 (s, 1H), 7.66 (dd, J = 1.3, 8.8 Hz, 1H), 7.48 (d, J = 8.6 Hz, 1H), 6.88 (d, J = 17.6 Hz, 1H), 4.78 (br t, J = 6.2 Hz, 1H), 4.20-4.13 (m, 2H), 3.89 (br d, J = 12.9 Hz, 1H), 3.66 (s, 3H), 3.56 (br s, 2H), 3.53 (br s, 2H), 2.93-2.86 (m, 1H), 2.82 (dt, J = 3.6, 6.8 Hz, 2H), 2.74 (br dd, J = 8.1, 14.4 Hz, 1H), 2.53 (br s, 3H), 2.25 (s, 3H), 1.72-1.65 (m, 4H), 1.01 (d, J = 6.3 Hz, 3H) |
| 122 | 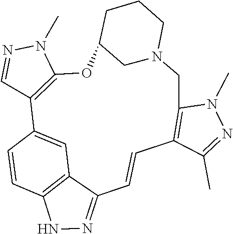 [SMILES: Cc1nn(C)c2c1/C=C/c1n[nH]c3ccc(cc13)-c1cnn(C)c1O[C@@H]1CCCN(C2)C1] | 430.2 | 1H NMR (400 MHz, METHANOL-d4) δ = 8.72- 8.49 (m, 1H), 7.90 (s, 1H), 7.72 (dd, J = 1.2, 8.8 Hz, 1H), 7.52 (s, 2H), 7.22-7.08 (m, 1H), 4.99 (br s, 1H), 4.62- 4.44 (m, 1H), 4.25-3.99 (m, 1H), 3.84 (s, 3H), 3.81 (s, 3H), 3.51-3.43 (m, 1H), 3.17-2.97 (m, 2H), 2.52 (s, 3H), 2.12- 1.99 (m, 2H), 1.94-1.84 (m, 2H), 1.70-1.54 (m, 1H) |
| 123 | 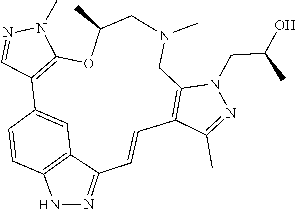 [SMILES: Cc1nn(C[C@H](C)O)c2c1/C=C/c1n[nH]c3ccc(cc13)-c1cnn(C)c1O[C@@H](C)CN(C)C2] | 462.2 | 1H NMR (400 MHz, DMSO- d6) δ = 12.87 (s, 1H), 8.47- 8.44 (m, 1H), 8.28 (d, J = 17.6 Hz, 1H), 7.88-7.84 (m, 1H), 7.67 (dd, J = 1.1, 8.8 Hz, 1H), 7.48 (d, J = 8.8 Hz, 1H), 6.89 (d, J = 17.6 Hz, 1H), 4.87 (d, J = 5.2 Hz, 1H), 4.81-4.73 (m, 1H), 4.01-3.89 (m, 4H), 3.86- 3.80 (m, 1H), 3.67 (s, 3H), 2.97-2.89 (m, 1H), 2.80 (dd, J = 7.9, 14.4 Hz, 1H), 2.40 (s, 3H), 2.29 (s, 3H), 1.09 (d, J = 6.0 Hz, 3H), 1.03 (d, J = 6.4 Hz, 3H) |
| 124 | 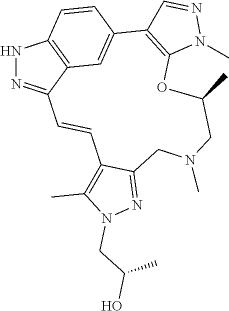 [SMILES: Cc1c2c(nn1C[C@H](C)O)CN(C)C[C@H](C)Oc1c(cnn1C)-c1ccc3[nH]nc(c3c1)/C=C/2] | 462.2 | 1H NMR (400 MHz, DMSO- d6) δ = 13.13-12.72 (m, 1H), 8.48 (s, 1H), 8.22-8.16 (m, 1H), 7.88-7.83 (m, 1H), 7.66 (dd, J = 1.4, 8.8 Hz, 1H), 7.48 (d, J = 8.8 Hz, 1H), 6.91-6.82 (m, 1H), 4.91 (br s, 1H), 4.78 (br t, J = 6.3 Hz, 1H), 4.04- 3.83 (m, 4H), 3.66 (s, 3H), 3.60-3.51 (m, 2H), 2.95-2.85 (m, 1H), 2.75 (dd, J = 7.9, 14.4 Hz, 1H), 2.54 (s, 2H), 2.25 (s, 3H), 1.06 (br d, J = 5.8 Hz, 3H), 1.01 (d, J = 6.4 Hz, 3H) |
| 125 | 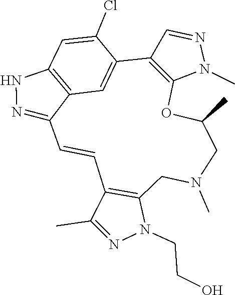 [SMILES: Cc1nn(CCO)c2c1/C=C/c1n[nH]c3cc(Cl)c(cc13)-c1cnn(C)c1O[C@@H](C)CN(C)C2] | 482.2 | 1H NMR (400 MHz, DMSO- d6) δ = 13.43-12.75 (m, 1H), 8.42 (br s, 1H), 7.88 (br s, 1H), 7.71 (br s, 1H), 7.35-7.22 (m, 1H), 7.00-6.90 (m, 1H), 5.25- 4.54 (m, 3H), 4.29-4.11 (m, 3H), 3.73 (br s, 6H), 2.91- 2.53 (m, 4H), 2.43 (br s, 3H), 0.99 (br d, J = 3.6 Hz, 3H) |
| 126 | 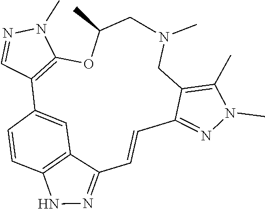 [SMILES: Cc1c2c(nn1C)/C=C/c1n[nH]c3ccc(cc13)-c1cnn(C)c1O[C@@H](C)CN(C)C2] | 418.2 | 1H NMR (400 MHz, METHANOL-d4) δ = 8.62 (s, 1H), 7.88 (s, 1H), 7.73 (br d, J = 8.8 Hz, 1H), 7.57 (d, J = 8.8 Hz, 1H), 7.54 (d, J = 1.6 Hz, 2H), 5.60-5.42 (m, 1H), 4.17- 4.04 (m, 1H), 3.94-3.84 (m, 5H), 3.79 (s, 3H), 3.71 (br d, J = 14.0 Hz, 1H), 3.01 (br s, 3H), 2.45 (s, 3H), 1.21 (d, J = 6.4 Hz, 3H) |
| 127 | 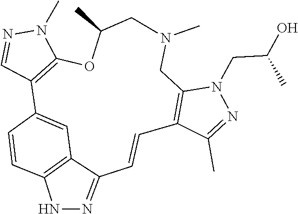 | 462.2 | 1H NMR (400 MHz, DMSO- d6) δ = 12.86 (s, 1H), 8.52- 8.45 (m, 1H), 8.32 (d, J = 17.6 Hz, 1H), 7.86 (s, 1H), 7.67 (d, J = 8.8 Hz, 1H), 7.48 (d, J = 8.8 Hz, 1H), 6.90 (d, J = 17.6 Hz, 1H), 4.90 (br d, J = 2.8 Hz, 1H), 4.80-4.68 (m, 1H), 4.00-3.87 (m, 5H), 3.67 (s, 3H), 2.98-2.90 (m, 1H), 2.86-2.75 (m, 1H), 2.40 (s, 3H), 2.29 (s, 3H), 1.05 (br d, J = 6.0 Hz, 6H) |
| 128 | 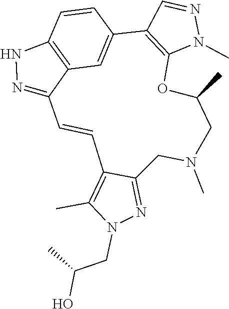 [SMILES: Cc1c2c(nn1C[C@@H](C)O)CN(C)C[C@H](C)Oc1c(cnn1C)-c1ccc3[nH]nc(c3c1)/C=C/2] | 462.3 | 1H NMR (400 MHz, DMSO- d6) δ = 12.87 (s, 1H), 8.47 (s, 1H), 8.20 (d, J = 17.6 Hz, 1H), 7.84 (s, 1H), 7.66 (d, J = 8.4 Hz, 1H), 7.48 (d, J = 8.8 Hz, 1H), 6.91-6.84 (m, 1H), 4.89 (d, J = 4.4 Hz, 1H), 4.83-4.74 (m, 1H), 4.01-3.86 (m, 4H), 3.69-3.65 (m, 3H), 3.54 (d, J = 12.8 Hz, 1H), 2.95-2.85 (m, 1H), 2.74 (dd, J = 8.0, 14.8 Hz, 1H), 2.49-2.47 (m, 3H), 2.26 (s, 3H), 1.07 (br d, J = 5.6 Hz, 3H), 1.01 (d, J = 6.4 Hz, 3H) |
| 129 | 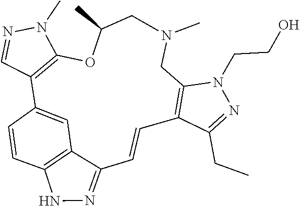 [SMILES: CCc1nn(CCO)c2c1/C=C/c1n[nH]c3ccc(cc13)-c1cnn(C)c1O[C@@H](C)CN(C)C2] | | (400 MHz, DMSO-d6) δ = 12.87 (s, 1H), 8.46 (s, 1H), 8.26 (d, J = 17.5 Hz, 1H), 7.85 (s, 1H), 7.69-7.64 (m, 1H), 7.48 (d, J = 8.6 Hz, 1H), 6.87 (d, J = 17.6 Hz, 1H), 4.87 (br t, J = 5.1 Hz, 1H), 4.81- 4.73 (m, 1H), 4.16-4.10 (m, 2H), 3.98-3.93 (m, 1H), 3.87- 3.82 (m, 1H), 3.71-3.68 (m, 1H), 3.67 (s, 3H), 3.64 (br d, J = 4.8 Hz, 1H), 2.96-2.90 (m, 1H), 2.85-2.82 (m, 1H), 2.81-2.77 (m, 2H), 2.29 (s, 3H), 1.28 (t, J = 7.4 Hz, 3H), 1.03 (d, J = 6.3 Hz, 3H |
| 130 | 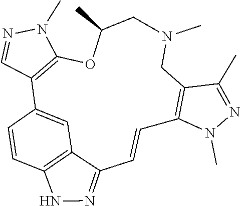 [SMILES: Cc1nn(C)c2c1CN(C)C[C@H](C)Oc1c(cnn1C)-c1ccc3[nH]nc(c3c1)/C=C/2] | 418.0 | 1H NMR (400 MHz, DMSO- d6) δ = 13.41-13.17 (m, 1H), 9.87-9.73 (m, 1H), 8.55 (s, 1H), 7.95 (s, 1H), 7.75 (br d, J = 8.8 Hz, 1H), 7.59 (d, J = 8.8 Hz, 1H), 7.56-7.49 (m, 1H), 7.36-7.29 (m, 1H), 5.53-5.43 (m, 1H), 4.05 (s, 3H), 3.94- 3.87 (m, 2H), 3.75 (s, 3H), 3.64-3.53 (m, 2H), 2.87 (br d, J = 4.0 Hz, 3H), 2.24 (s, 3H), 1.07 (br d, J = 6.4 Hz, 3H). |
| 131 | 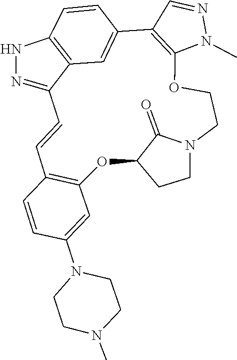 [SMILES: CN1CCN(c2ccc3c(c2)O[C@@H]2CCN(CCOc4c(cnn4C)-c4ccc5[nH]nc(c5c4)/C=C/3)C2=O)CC1] | 540.2 | 1H NMR (400 MHz, METHANOL-d4) δ = 8.15 (s, 1H), 7.68 (s, 1H), 7.62-7.58 (m, 2H), 7.57-7.53 (m, 1H), 7.46-7.39 (m, 1H), 7.13 (d, J = 8.5 Hz, 1H), 6.84-6.75 (m, 2H), 6.49 (dd, J = 2.2, 8.4 Hz, 1H), 5.11-5.03 (m, 1H), 4.34 (s, 2H), 4.13-4.04 (m, 2H), 3.99-3.90 (m, 2H), 3.84 (s, 3H), 3.68-3.59 (m, 2H), 3.57- 3.47 (m, 2H), 3.41-3.34 (m, 1H), 3.17 (br s, 2H), 3.00- 2.95 (m, 3H), 2.42-2.28 (m, 1H), 2.23-2.14 (m, 1H) |
| 132 | 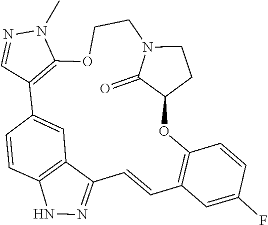 [SMILES: Cn1ncc2c1OCCN1CC[C@@H](Oc3ccc(F)cc3/C=C/c3n[nH]c4ccc-2cc34)C1=O] | 460.0 | 1H NMR (400 MHz, DMSO- d6) δ = 13.25 (br s, 1H), 8.04 (s, 1H), 7.73 (s, 1H), 7.68 (s, 1H), 7.64-7.58 (m, 3H), 7.52- 7.46 (m, 1H), 7.27 (dd, J = 4.78, 9.6 Hz, 1H), 7.12 (dt, J = 3.2, 8.4 Hz, 1H), 5.00 (d, J = 6.0 Hz, 1H), 4.27 (br t, J = 8.0 Hz, 1H), 4.20-4.09 (m, 1H), 4.08-3.98 (m, 1H), 3.91-3.84 (m, 1H), 3.77 (s, 3H), 3.50 (br t, J = 8.4 Hz, 1H), 3.40 (br d, J = 8.4 Hz, 1H), 2.38-2.26 (m, 1H), 2.09-1.98 (m, 1H) |
| 133 | 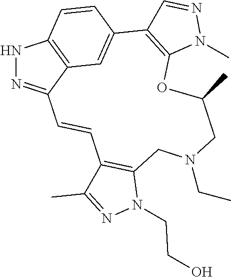 [SMILES: CCN1Cc2c(c(C)nn2CCO)/C=C/c2n[nH]c3ccc(cc23)-c2cnn(C)c2O[C@@H](C)C1] | 462.3 | 1H NMR (400 MHz, DMSO- d6) δ = 12.85 (s, 1H), 8.53 (s, 1H), 8.11 (d, J = 17.5 Hz, 1H), 7.84 (s, 1H), 7.64 (dd, J = 1.2, 8.7 Hz, 1H), 7.45 (d, J = 8.6 Hz, 1H), 6.88 (d, J = 17.5 Hz, 1H), 4.84 (t, J = 5.3 Hz, 1H), 4.75-4.66 (m, 1H), 4.14-4.05 (m, 2H), 3.98-3.86 (m, 2H), 3.65 (s, 5H), 2.95-2.89 (m, 1H), 2.88-2.80 (m, 1H), 2.59- 2.51 (m, 2H), 2.38 (s, 3H), 1.02 (d, J = 6.3 Hz, 3H), 0.89 (t, J = 7.1 Hz, 3H) |
| 134 | 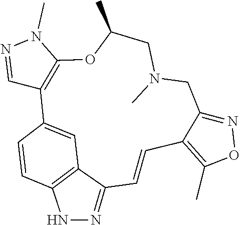 [SMILES: Cc1onc2c1/C=C/c1n[nH]c3ccc(cc13)-c1cnn(C)c1O[C@@H](C)CN(C)C2] | 405.0 | ¹H NMR (500 MHz, DMSO- d6) δ = 13.06 (s, 1H), 8.45 (s, 1H), 8.03 (d, J = 17.5 Hz, 1H), 7.87 (s, 1H), 7.70 (dd, J = 1.4, 8.8 Hz, 1H), 7.52 (d, J = 8.8 Hz, 1H), 7.09 (d, J = 17.5 Hz, 1H), 4.81 (br t, J = 6.2 Hz, 1H), 3.99 (d, J = 13.4 Hz, 1H), 3.73 (d, J = 13.4 Hz, 1H), 3.67 (s, 3H), 2.98-2.91 (m, 1H), 2.80 (dd, J = 8.2, 14.5 Hz, 1H), 2.67 (s, 3H), 2.30 (s, 3H), 1.03- 0.99 (m, 3H) |
| 135 | 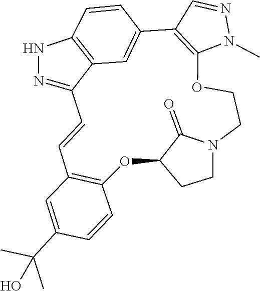 [SMILES: Cn1ncc2c1OCCN1CC[C@@H](Oc3ccc(C(C)(C)O)cc3/C=C/c3n[nH]c4ccc-2cc34)C1=O] | 500.2 | 1H NMR (400 MHz, DMSO- d6) δ = 13.17 (s, 1H), 8.06 (s, 1H), 7.76-7.73 (m, 2H), 7.63- 7.56 (m, 3H), 7.55 (s, 1H), 7.38 (dd, J = 2.4, 8.6 Hz, 1H), 7.15 (d, J = 8.6 Hz, 1H), 5.06- 4.95 (m, 2H), 4.29 (t, J = 8.4 Hz, 1H), 4.19-4.11 (m, 1H), 4.03 (dt, J = 2.8, 5.2 Hz, 1H), 3.94-3.87 (m, 1H), 3.77 (s, 3H), 3.51 (t, J = 8.8 Hz, 1H), 3.44-3.39 (m, 1H), 2.32 (d, J = 8.0 Hz, 1H), 2.09-1.97 (m, 1H), 1.47 (s, 6H) |
| 136 | 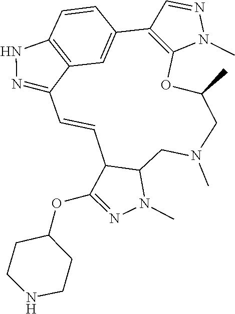 [SMILES: C[C@H]1CN(C)CC2C(/C=C/c3n[nH]c4ccc(cc34)-c3cnn(C)c3O1)C(OC1CCNCC1)=NN2C] | 503.3 | 1H NMR (400 MHz, METHANOL-d4) δ = 8.59- 8.50 (m, 2H), 8.30-8.20 (m, 1H), 7.84 (s, 1H), 7.70 (br d, J = 8.8 Hz, 1H), 7.53-7.47 (m, 1H), 7.29-7.21 (m, 1H), 4.00- 3.91 (m, 1H), 3.81 (br d, J = 14.5 Hz, 1H), 3.73 (br d, J = 5.9 Hz, 6H), 3.52-3.40 (m, 2H), 3.00-2.83 (m, 2H), 2.42 (s, 3H), 2.28-2.17 (m, 4H), 1.12 (br d, J = 5.8 Hz, 3H) |
| 137 | 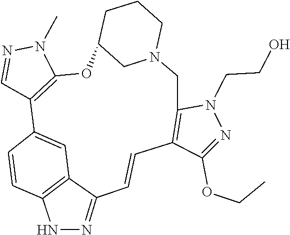 [SMILES: CCOc1nn(CCO)c2c1/C=C/c1n[nH]c3ccc(cc13)-c1cnn(C)c1O[C@@H]1CCCN(C2)C1] | 490.3 | 1H NMR (400 MHz, DMSO- d6) δ = 12.82-12.72 (m, 1H), 8.86-8.72 (m, 1H), 8.17-8.03 (m, 1H), 7.90 (s, 1H), 7.71- 7.61 (m, 1H), 7.48-7.39 (m, 1H), 7.20 (s, 1H), 4.94 (br s, 1H), 4.32-4.24 (m, 2H), 4.06- 3.92 (m, 4H), 3.78 (s, 3H), 3.69-3.57 (m, 4H), 2.85-2.64 (m, 2H), 1.67-1.52 (m, 2H), 1.41 (t, J = 7.2 Hz, 4H), 1.36- 1.22 (m, 2H) |
| 138 | 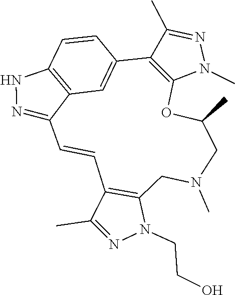 [SMILES: Cc1nn(CCO)c2c1/C=C/c1n[nH]c3ccc(cc13)-c1c(C)nn(C)c1O[C@@H](C)CN(C)C2] | 462.2 | 1H NMR (400 MHz, DMSO- d6) δ = 12.85 (br s, 1H), 8.36 (s, 1H), 8.21-8.11 (m, 1H), 7.63-7.44 (m, 2H), 6.87 (d, J = 16.0 Hz, 1H), 4.97-4.78 (m, 1H), 4.73-4.63 (m, 1H), 4.17- 4.04 (m, 2H), 3.88 (s, 2H), 3.68 (br d, J = 4.0 Hz, 2H), 3.61 (s, 3H), 2.89 (br d, J = 16.0 Hz, 1H), 2.73 (br dd, J = 8.0, 14.4 Hz, 1H), 2.40 (s, 3H), 2.33 (s, 3H), 2.22 (s, 3H), 0.95 (br d, J = 6.0 Hz, 3H) |
| 139 | 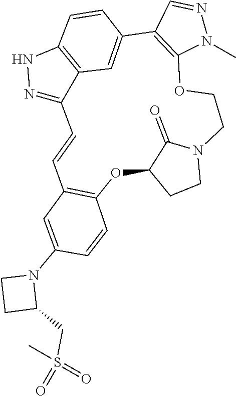 [SMILES: Cn1ncc2c1OCCN1CC[C@@H](Oc3ccc(N4CC[C@H]4CS(C)(=O)=O)cc3/C=C/c3n[nH]c4ccc-2cc34)C1=O] | 589.3 | 1H NMR (400 MHz, DMSO- d6) δ = 13.78-12.78 (m, 1H), 8.05 (s, 1H), 7.77-7.69 (m, 1H), 7.61-7.54 (m, 2H), 7.53- 7.44 (m, 2H), 7.25-7.09 (m, 1H), 7.07-6.96 (m, 1H), 6.08- 5.91 (m, 1H), 5.76 (td, J = 7.2, 16.0 Hz, 1H), 4.82 (br d, J = 6.0 Hz, 1H), 4.27-4.11 (m, 2H), 4.11-4.04 (m, 1H), 3.91 (br d, J = 7.2 Hz, 2H), 3.87- 3.74 (m, 6H), 3.00-2.92 (m, 2H), 2.87 (s, 3H), 2.22-2.14 (m, 1H), 2.07 (s, 2H), 2.00- 1.93 (m, 1H) |
| 140 | 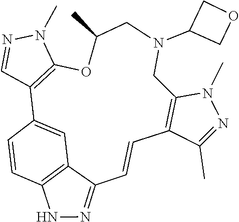 [SMILES: Cc1nn(C)c2c1/C=C/c1n[nH]c3ccc(cc13)-c1cnn(C)c1O[C@@H](C)CN(C1COC1)C2] | 460.2 | 1H NMR (400 MHz, DMSO- d6) δ = 12.91 (s, 1H), 8.55 (s, 1H), 7.89 (s, 1H), 7.76 (d, J = 17.2 Hz, 1H), 7.69 (d, J = 8.8 Hz, 1H), 7.49 (d, J = 8.8 Hz, 1H), 6.93 (d, J = 17.2 Hz, 1H), 4.96-4.81 (m, 1H), 4.68-4.48 (m, 2H), 4.27-4.21 (m, 1H), 4.17-4.06 (m, 2H), 3.90-3.83 (m, 1H), 3.73 (s, 3H), 3.70- 3.64 (m, 4H), 3.10-2.99 (m, 1H), 2.91-2.80 (m, 1H), 2.38 (s, 3H), 1.07-1.03 (m, 3H) |
| 141 | 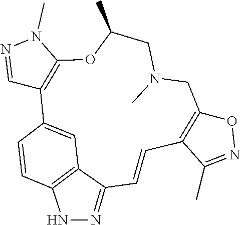 [SMILES: Cc1noc2c1/C=C/c1n[nH]c3ccc(cc13)-c1cnn(C)c1O[C@@H](C)CN(C)C2] | 405.1 | 1H NMR (499 MHz, DMSO- d6) δ = 13.06 (s, 1H), 8.47 (s, 1H), 8.19 (d, J = 17.5 Hz, 1H), 7.90 (s, 1H), 7.72 (dd, J = 1.4, 8.8 Hz, 1H), 7.52 (d, J = 8.5 Hz, 1H), 7.11 (d, J = 17.5 Hz, 1H), 4.74 (br t, J = 5.9 Hz, 1H), 4.15 (d, J = 15.3 Hz, 1H), 3.92 (br d, J = 15.3 Hz, 1H), 3.68 (s, 3H), 3.00-2.94 (m, 1H), 2.92-2.83 (m, 1H), 2.52 (s, 3H), 2.36 (s, 3H), 1.08 (d, J = 6.3 Hz, 3H) |
| 142 | 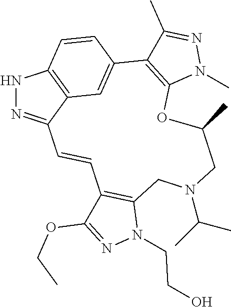 [SMILES: CCOc1nn(CCO)c2c1/C=C/c1n[nH]c3ccc(cc13)-c1c(C)nn(C)c1O[C@@H](C)CN(C(C)C)C2] | 520.3 | 1H NMR (400 MHz, DMSO- d6) δ = 12.77 (s, 1H), 8.47 (br s, 1H), 7.89 (br d, J = 16.0 Hz, 1H), 7.55-7.47 (m, 2H), 7.08 (br d, J = 16.0 Hz, 1H), 4.84 (br t, J = 6.0 Hz, 1H), 4.44 (br s, 1H), 4.28 (q, J = 8.0 Hz, 2H), 4.07-3.95 (m, 5H), 3.63 (s, 3H), 2.83-2.65 (m, 4H), 2.35 (s, 3H), 1.42 (t, J = 8.0 Hz, 3H), 0.94 (br d, J = 8.0 Hz, 3H), 0.85 (br t, J = 8.0 Hz, 6H) |
| 143 | 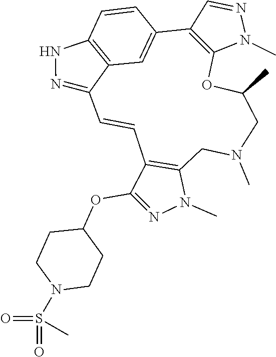 [SMILES: C[C@H]1CN(C)Cc2c(c(OC3CCN(S(C)(=O)=O)CC3)nn2C)/C=C/c2n[nH]c3ccc(cc23)-c2cnn(C)c2O1] | 581.3 | 1H NMR (400 MHz, METHANOL-d4) δ = 8.56 (s, 1H), 8.28-8.20 (m, 1H), 7.85- 7.82 (m, 1H), 7.71-7.66 (m, 1H), 7.49 (d, J = 9.1 Hz, 1H), 7.29 (d, J = 17.1 Hz, 1H), 4.99- 4.95 (m, 2H), 4.59-4.55 (m, 1H), 4.00-3.94 (m, 1H), 3.82 (br d, J = 14.5 Hz, 1H), 3.74 (d, J = 7.0 Hz, 6H), 3.47 (br dd, J = 5.6, 10.3 Hz, 2H), 3.02- 2.90 (m, 2H), 2.88 (s, 3H), 2.44 (s, 3H), 2.07 (br s, 4H), 1.45-1.41 (m, 1H), 1.13 (d, J = 6.1 Hz, 3H) |
| 144 | 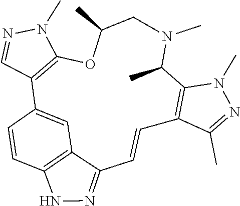 [SMILES: Cc1nn(C)c2c1/C=C/c1n[nH]c3ccc(cc13)-c1cnn(C)c1O[C@@H](C)CN(C)[C@@H]2C] | 432.2 | 1H NMR (400 MHz, DMSO- d6) δ = 12.86 (br s, 1H), 8.59- 8.43 (m, 2H), 7.86 (s, 1H), 7.67 (br d, J = 8.4 Hz, 1H), 7.48 (d, J = 8.4 Hz, 1H), 6.84 (d, J = 17.6 Hz, 1H), 4.61 (br d, J = 6.0 Hz, 1H), 3.84 (q, J = 6.4 Hz, 1H), 3.74 (s, 3H), 3.67 (s, 3H), 2.63-2.52 (m, 2H), 2.41-2.33 (m, 6H), 1.37 (br d, J = 6.4 Hz, 3H), 1.05 (br d, J = 6.0 Hz, 3H) |
| 145 | 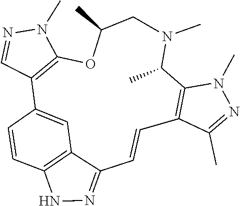 [SMILES: Cc1nn(C)c2c1/C=C/c1n[nH]c3ccc(cc13)-c1cnn(C)c1O[C@@H](C)CN(C)[C@H]2C] | 432.2 | 1H NMR (400 MHz, DMSO- d6) δ = 13.16-12.59 (m, 1H), 8.70 (s, 1H), 8.47 (br d, J = 17.6 Hz, 1H), 7.90 (s, 1H), 7.69 (br d, J = 8.4 Hz, 1H), 7.46 (br d, J = 8.4 Hz, 1H), 6.90 (br d, J = 17.6 Hz, 1H), 4.45 (br s, 1H), 3.90-3.82 (m, 1H), 3.75 (s, 3H), 3.70 (s, 3H), 3.28-3.23 (m, 1H), 2.68 (br d, J = 13.2 Hz, 1H), 2.37 (s, 3H), 2.30 (s, 3H), 1.41 (br d, J = 6.0 Hz, 3H), 1.18 (br d, J = |
| | | | 6.0 Hz, 3H) |
| 146 | 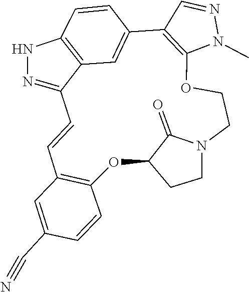 [SMILES: Cn1ncc2c1OCCN1CC[C@@H](Oc3ccc(C#N)cc3/C=C/c3n[nH]c4ccc-2cc34)C1=O] | 467.1 | 1H NMR (400 MHz, METHANOL-d4) δ = 8.15 (s, 1H), 8.02 (d, J = 2.0 Hz, 1H), 7.71-7.69 (m, 1H), 7.65 (br d, J = 2.4 Hz, 1H), 7.62 (s, 1H), 7.59-7.55 (m, 1H), 7.49-7.46 (m, 1H), 7.36-7.31 (m, 2H), 5.17 (br d, J = 6.4 Hz, 1H), 4.27-4.15 (m, 2H), 4.00 (td, J = 3.2, 6.8 Hz, 1H), 3.85 (s, 3H), 3.71-3.65 (m, 1H), 3.61- 3.50 (m, 2H), 2.54 (dt, J = 8.0, 14.4 Hz, 1H), 2.32-2.21 (m, 1H) |
| 147 | 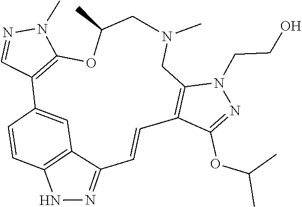 | 492.2 | 1H NMR (400 MHz, DMSO- d6) δ = 12.77 (br s, 1H), 8.49- 8.39 (m, 1H), 8.12 (d, J = 17.1 Hz, 1H), 7.86 (s, 1H), 7.65 (dd, J = 1.1, 8.8 Hz, 1H), 7.45 (d, J = 8.6 Hz, 1H), 7.15 (d, J = 17.1 Hz, 1H), 4.95-4.86 (m, 1H), 4.86-4.79 (m, 1H), 4.74 (br dd, J = 5.5, 6.5 Hz, 1H), 4.08-3.96 (m, 2H), 3.96- 3.88 (m, 1H), 3.86-3.77 (m, 1H), 3.67 (s, 3H), 3.65-3.62 (m, 2H), 3.01-2.89 (m, 1H), 2.80 (br dd, J = 7.0, 14.6 Hz, 1H), 2.36- 2.31 (m, 3H), 1.38 (dd, J = 6.3, 8.0 Hz, 6H), 1.05 (d, J = 6.4 Hz, 3H) |
| 148 | 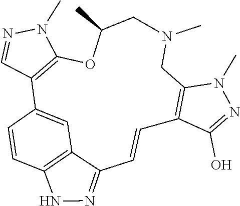 [SMILES: C[C@H]1CN(C)Cc2c(c(O)nn2C)/C=C/c2n[nH]c3ccc(cc23)-c2cnn(C)c2O1] | 420.2 | 1H NMR (400 MHz, METHANOL-d4) δ = 8.52 (s, 1H), 8.12 (br d, J = 16.8 Hz, 1H), 7.80 (s, 1H), 7.67-7.62 (m, 1H), 7.49-7.44 (m, 1H), 7.31 (d, J = 16.8 Hz, 1H), 3.71 (s, 3H), 3.64 (s, 3H), 3.63- 3.58 (m, 1H), 2.96-2.88 (m, 2H), 2.42 (br s, 3H), 1.86 (td, J = 3.2, 6.8 Hz, 2H), 1.11-1.07 (m, 3H) |
| 149 | 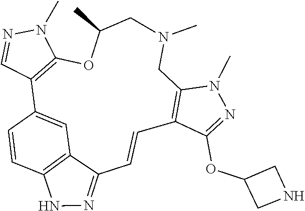 | 475.3 | 1H NMR (400 MHz, METHANOL-d4) δ = 8.57- 8.55 (m, 1H), 8.26 (d, J = 17.2 Hz, 1H), 7.85 (s, 1H), 7.71 (dd, J = 0.8, 8.8 Hz, 1H), 7.51 (d, J = 8.8 Hz, 1H), 7.32- 7.25 (d, J = 17.2 Hz, 1H), 5.44- 5.34 (m, 1H), 4.56 (br d, J = 11.6 Hz, 3H), 4.31-4.25 (m, 2H), 4.00-3.94 (m, 1H), 3.83 (br d, J = 14.4 Hz, 1H), 3.75 (s, 3H), 3.71 (s, 3H), 3.01- 2.88 (m, 2H), 2.43 (s, 3H), 1.13 (d, J = 6.4 Hz, 3H) |
| 150 | 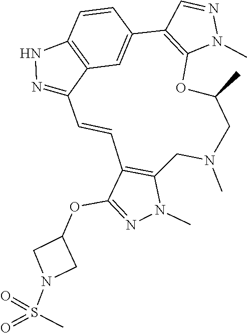 [SMILES: C[C@H]1CN(C)Cc2c(c(OC3CN(S(C)(=O)=O)C3)nn2C)/C=C/c2n[nH]c3ccc(cc23)-c2cnn(C)c2O1] | 553.3 | 1H NMR (400 MHz, METHANOL-d4) δ = 8.56- 8.52 (s, 1H), 8.26-8.18 (d, J = 12.8 Hz, 1H), 7.85-7.82 (s, 1H), 7.72-7.66 (m, 1H), 7.52- 7.47 (m, 1H), 7.30-7.23 (d, J = 12.8 Hz, 1H), 5.30-5.22 (m, 1H), 4.91-4.88 (m, 1H), 4.40- 4.33 (m, 2H), 4.12 (dd, J = 4.8, 9.6 Hz, 2H), 3.96-3.77 (m, 2H), 3.73 (s, 3H), 3.71- 3.68 (m, 3H), 3.05-3.01 (m, 3H), 2.98-2.84 (m, 2H), 2.44- 2.39 (m, 3H), 1.11 (d, J = 6.4 Hz, 3H) |
| 151 | 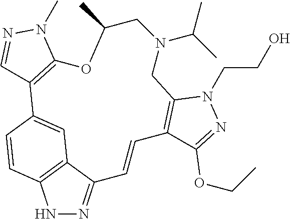 [SMILES: CCOc1nn(CCO)c2c1/C=C/c1n[nH]c3ccc(cc13)-c1cnn(C)c1O[C@@H](C)CN(C(C)C)C2] | 506.2 | 1H NMR (400 MHz, METHANOL-d4) δ = 8.70 (s, 1H), 8.12-8.04 (d, J = 16.8 Hz, 1H), 7.84-7.81 (m, 1H), 7.69-7.63 (m, 1H), 7.51-7.46 (m, 1H), 7.33-7.26 (d, J = 16.8 Hz, 1H), 4.79-4.72 (m, 1H), 4.57 (br s, 1H), 4.35 (q, J = 7.2 Hz, 2H), 4.19-4.11 (m, 2H), 4.06-4.00 (m, 2H), 3.88- 3.82 (m, 2H), 3.76 (s, 3H), 2.91-2.84 (m, 2H), 1.50 (t, J = 7.2 Hz, 3H), 1.15-1.11 (m, 3H), 1.03 (br d, J = 6.4 Hz, |
| | | | 3H), 0.99 (d, J = 6.4 Hz, 3H) |
| 152 | 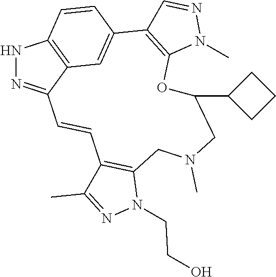 [SMILES: Cc1nn(CCO)c2c1/C=C/c1n[nH]c3ccc(cc13)-c1cnn(C)c1OC(C1CCC1)CN(C)C2] | 488.4 | 1H NMR (400 MHz, DMSO- d6) δ = 12.88 (s, 1H), 8.57- 8.41 (m, 1H), 8.29 (br d, J = 17.6 Hz, 1H), 7.77 (s, 1H), 7.64 (d, J = 8.4 Hz, 1H), 7.49 (d, J = 8.8 Hz, 1H), 6.90 (d, J = 17.6 Hz, 1H), 4.86 (br s, 1H), 4.70-4.50 (m, 1H), 4.09 (br d, J = 4.8 Hz, 2H), 3.95 (br d, J = 14.0 Hz, 1H), 3.86-3.75 (m, 1H), 3.70 (s, 3H), 3.67 (br s, 2H), 2.79-2.68 (m, 2H), 2.40 (s, 3H), 2.26 (br s, 3H), 1.82 (br d, J = 2.4 Hz, 1H), 1.76- 1.60 (m, 2H), 1.52-1.37 (m, 2H), 1.32-1.20 (m, 2H) |
| 153 | 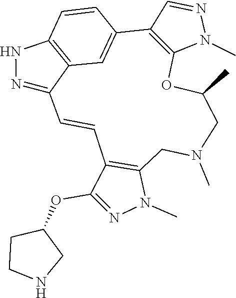 [SMILES: C[C@H]1CN(C)Cc2c(c(O[C@H]3CCNC3)nn2C)/C=C/c2n[nH]c3ccc(cc23)-c2cnn(C)c2O1] | 488.3 | 1H NMR (400 MHz, METHANOL-d4) δ = 8.57 (s, 1H), 7.86 (s, 1H), 7.79-7.69 (m, 2H), 7.53 (d, J = 9.2 Hz, 1H), 7.33-7.25 (m, 1H), 5.51 (br s, 1H), 5.24-5.17 (m, 2H), 4.64-4.55 (m, 1H), 4.33-4.23 (m, 1H), 3.84 (s, 3H), 3.77 (s, 3H), 3.68-3.53 (m, 4H), 3.50- 3.40 (m, 2H), 2.83 (s, 3H), 2.57-2.47 (m, 1H), 2.45-2.35 (m, 1H), 1.18 (d, J = 6.4 Hz, 3H) |
| 154 | 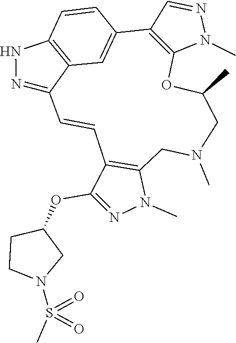 [SMILES: C[C@H]1CN(C)Cc2c(c(O[C@H]3CCN(S(C)(=O)=O)C3)nn2C)/C=C/c2n[nH]c3ccc(cc23)-c2cnn(C)c2O1] | 567.2 | 1H NMR (400 MHz, METHANOL-d4) δ = 8.53 (s, 1H), 8.22 (d, J = 17.2 Hz, 1H), 7.83 (s, 1H), 7.69 (d, J = 9.2 Hz, 1H), 7.49 (d, J = 9.2 Hz, 1H), 7.21 (d, J = 17.2 Hz, 1H), 5.36 (s, 2H), 3.98 (d, J = 14.4 Hz, 1H), 3.82 (d, J = 14.4 Hz, 1H), 3.69-3.67 (m, 2H), 3.49-3.46 (m, 1H), 3.14- 3.11 (m, 1H), 3.02 (q, J = 7.2 Hz, 5H), 2.97-2.94 (m, 1H), 2.93- 2.90 (m, 1H), 2.88 (s, 3H), 2.67 (s, 3H), 2.43 (s, 3H), 1.12 (d, J = 6.0 Hz, 3H) |
| 155 | 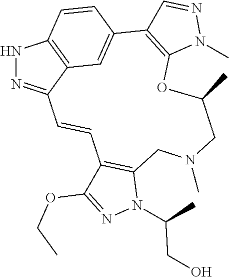 [SMILES: CCOc1nn([C@@H](C)CO)c2c1/C=C/c1n[nH]c3ccc(cc13)-c1cnn(C)c1O[C@@H](C)CN(C)C2] | 492.3 | 1H NMR (400 MHz, METHANOL-d4) δ = 8.63- 8.54 (m, 1H), 7.89-7.82 (m, 1H), 7.73-7.67 (m, 1H), 7.59- 7.49 (m, 2H), 7.42-7.34 (m, 1H), 5.44-5.30 (m, 1H), 4.61- 4.48 (m, 2H), 4.42 (dd, J = 1.6, 7.2 Hz, 2H), 3.85-3.58 (m, 8H), 3.02 (s, 3H), 1.54- 1.45 (m, 6H), 1.19 (d, J = 6.4 Hz, 3H) |
| 156 | 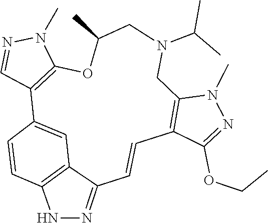 [SMILES: CCOc1nn(C)c2c1/C=C/c1n[nH]c3ccc(cc13)-c1cnn(C)c1O[C@@H](C)CN(C(C)C)C2] | 476.0 | 1H NMR (400 MHz, METHANOL-d4) δ = 8.77- 8.61 (m, 1H), 7.98 (d, J = 17.0 Hz, 1H), 7.83 (s, 1H), 7.69- 7.64 (m, 1H), 7.48 (d, J = 8.6 Hz, 1H), 7.32-7.25 (m, 1H), 4.80-4.72 (m, 1H), 4.61-4.51 (m, 1H), 4.36-4.29 (m, 2H), 3.98 (s, 2H), 3.76 (d, J = 2.0 Hz, 6H), 2.89-2.78 (m, 2H), 1.49 (t, J = 7.1 Hz, 3H), 1.16-1.09 (m, 3H), 1.07-0.99 (m, 6H) |
| 157 | 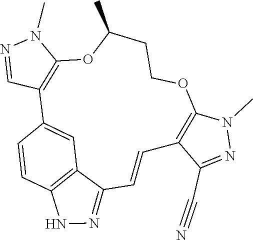 [SMILES: C[C@H]1CCOc2c(c(C#N)nn2C)/C=C/c2n[nH]c3ccc(cc23)-c2cnn(C)c2O1] | 416.0 | 1H NMR (499 MHz, DMSO- d6) δ = 13.16 (s, 1H), 8.53 (s, 1H), 7.96 (s, 1H), 7.74 (dd, J = 1.4, 8.8 Hz, 1H), 7.54 (d, J = 8.8 Hz, 1H), 7.38 (s, 1H), 7.38- 7.33 (m, 1H), 4.90-4.85 (m, 1H), 4.75-4.68 (m, 1H), 4.51 (td, J = 5.4, 10.3 Hz, 1H), 3.82 (s, 3H), 3.70 (s, 3H), 2.52 (d, J = 1.9 Hz, 1H), 2.44 (d, J = 5.2 Hz, 1H), 1.30 (d, J = 6.6 Hz, 3H) |
| 158 | 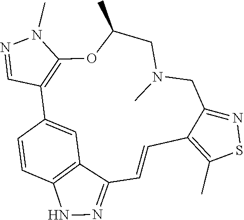 [SMILES: Cc1snc2c1/C=C/c1n[nH]c3ccc(cc13)-c1cnn(C)c1O[C@@H](C)CN(C)C2] | 421.0 | 1H NMR (499 MHz, DMSO- d6) δ = 13.12 (s, 1H), 8.47 (s, 1H), 8.44 (d, J = 17.5 Hz, 1H), 7.85 (s, 1H), 7.68 (dd, J = 1.4, 8.8 Hz, 1H), 7.53 (d, J = 8.5 Hz, 1H), 7.18 (d, J = 17.5 Hz, 1H), 4.90-4.84 (m, 1H), 4.06 (d, J = 12.9 Hz, 1H), 3.75 (d, J = 12.9 Hz, 1H), 3.67 (s, 3H), 2.87 (dd, J = 2.2, 14.5 Hz, 1H), 2.78-2.71 (m, 4H), 2.23 (s, 3H), 0.97 (d, J = 6.3 Hz, 3H) |
| 159 | 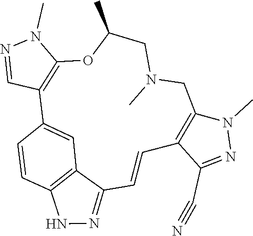 [SMILES: C[C@H]1CN(C)Cc2c(c(C#N)nn2C)/C=C/c2n[nH]c3ccc(cc23)-c2cnn(C)c2O1] | 429.0 | 1H NMR (400 MHz, DMSO- d6) δ = 8.47 (s, 1H), 8.25 (d, J = 17.4 Hz, 1H), 7.90 (s, 1H), 7.72 (d, J = 8.7 Hz, 1H), 7.53 (d, J = 8.7 Hz, 1H), 7.45 (d, J = 17.6 Hz, 1H), 4.77 (br t, J = 6.1 Hz, 1H), 4.10-4.03 (m, 1H), 3.97-3.89 (m, 4H), 3.68 (s, 3H), 3.00-2.93 (m, 1H), 2.91-2.84 (m, 1H), 2.33 (s, 3H), 1.06 (d, J = 6.2 Hz, 3H |
| 160 | 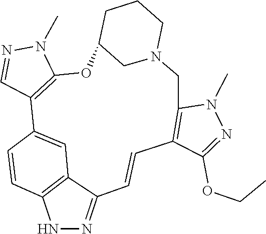 | 460.2 | 1H NMR (400 MHz, METHANOL-d4) δ = 8.90- 8.77 (m, 1H), 8.24-8.11 (m, 1H), 7.86 (s, 1H), 7.68 (dd, J = 1.4, 8.8 Hz, 1H), 7.48-7.45 (m, 1H), 7.44-7.39 (m, 1H), 4.58 (s, 1H), 4.35-4.29 (m, 2H), 3.99 (d, J = 15.6 Hz, 1H), 3.79 (s, 3H), 3.66 (s, 3H), 3.58- 3.53 (m, 1H), 2.91-2.78 (m, 2H), 2.09-1.94 (m, 2H), 1.77- 1.56 (m, 4H), 1.50 (t, J = 7.2 Hz, 3H) |
| 161 | 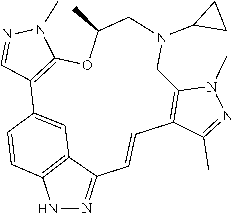 [SMILES: Cc1nn(C)c2c1/C=C/c1n[nH]c3ccc(cc13)-c1cnn(C)c1O[C@@H](C)CN(C1CC1)C2] | 444.3 | 1H NMR (400 MHz, DMSO- d6) δ = 12.87 (s, 1H), 8.50- 8.31 (m, 1H), 7.97-7.79 (m, 1H), 7.68-7.54 (m, 2H), 7.47 (d, J = 8.8 Hz, 1H), 6.90 (d, J = 17.2 Hz, 1H), 4.82-4.64 (m, 1H), 4.09 (d, J = 13.6 Hz, 1H), 3.83 (s, 3H), 3.68 (s, 3H), 3.19- 3.12 (m, 2H), 2.39 (s, 3H), 2.07 (s, 2H), 0.99 (d, J = 6.4 Hz, 3H), 0.38-0.29 (m, 2H), 0.28- -0.14 (m, 2H) |
| 162 | 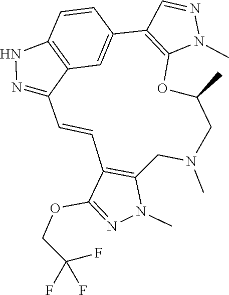 [SMILES: C[C@H]1CN(C)Cc2c(c(OCC(F)(F)F)nn2C)/C=C/c2n[nH]c3ccc(cc23)-c2cnn(C)c2O1] | 502.2 | 1H NMR (400 MHz, METHANOL-d4) δ = 8.56- 8.50 (s, 1H), 8.25-8.16 (d, J = 16.0 Hz, 1H), 7.82 (s, 1H), 7.72-7.65 (m, 1H), 7.49 (d, J = 8.8 Hz, 1H), 7.25-7.16 (d, J = 16.0 Hz, 1H), 4.91-4.90 (m, 1H), 4.83-4.73 (m, 2H), 3.99- 3.78 (m, 2H), 3.73 (s, 6H), 3.01-2.83 (m, 2H), 2.48-2.40 (s, 3H), 1.14-1.07 (m, 3H) |
| 163 | 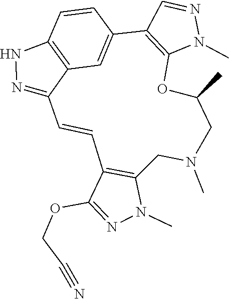 [SMILES: C[C@H]1CN(C)Cc2c(c(OCC#N)nn2C)/C=C/c2n[nH]c3ccc(cc23)-c2cnn(C)c2O1] | 459.2 | 1H NMR (400 MHz, METHANOL-d4) δ = 8.56- 8.50 (m, 1H), 8.26-8.18 (m, 1H), 7.83 (s, 1H), 7.68 (br d, J = 8.8 Hz, 1H), 7.49 (d, J = 8.8 Hz, 1H), 7.22-7.15 (m, 1H), 5.15-5.11 (m, 2H), 4.61-4.56 (m, 1H), 3.99-3.80 (m, 2H), 3.74 (d, J = 6.4 Hz, 5H), 3.00- 2.86 (m, 2H), 2.43 (s, 2H), 2.06-2.01 (m, 2H), 1.15-1.08 (m, 3H) |
| 164 | 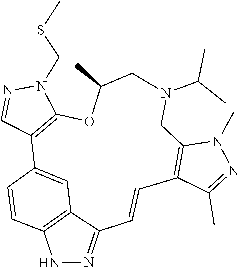 [SMILES: CSCn1ncc2c1O[C@@H](C)CN(C(C)C)Cc1c(c(C)nn1C)/C=C/c1n[nH]c3ccc-2cc13] | 492.2 | 1H NMR (400 MHz, METHANOL-d4) δ = 8.66 (s, 1H), 8.13 (d, J = 17.4 Hz, 1H), 7.89 (s, 1H), 7.69 (d, J = 9.0 Hz, 1H), 7.52 (d, J = 8.8 Hz, 1H), 7.02 (d, J = 17.5 Hz, 1H), 5.26 (br d, J = 14.0 Hz, 1H), 5.08 (br d, J = 13.6 Hz, 1H), 4.59 (s, 1H), 4.02 (s, 2H), 3.85 (s, 3H), 3.04-2.91 (m, 1H), 2.87-2.66 (m, 2H), 2.49 (s, 3H), 2.25 (s, 3H), 1.12 (d, J = 6.4 Hz, 3H), 1.03 (br d, J = 6.6 Hz, 3H), 0.99 (d, J = 6.5 Hz, 3H |
| 165 | 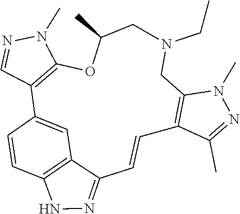 [SMILES: CCN1Cc2c(c(C)nn2C)/C=C/c2n[nH]c3ccc(cc23)-c2cnn(C)c2O[C@@H](C)C1] | 432.3 | 1H NMR (400 MHz, DMSO- d6) δ = 12.87 (s, 1H), 8.55 (s, 1H), 8.05 (d, J = 17.6 Hz, 1H), 7.86 (s, 1H), 7.71-7.61 (m, 1H), 7.47 (d, J = 8.8 Hz, 1H), 6.91 (d, J = 17.6 Hz, 1H), 4.81- 4.66 (m, 1H), 4.01-3.81 (m, 2H), 3.76 (s, 3H), 3.68 (s, 3H), 3.02-2.80 (m, 2H), 2.60-2.53 (m, 2H), 2.39 (s, 3H), 1.04 (d, J = 6.4 Hz, 3H), 0.99-0.89 (m, 3H) |
| 166 | 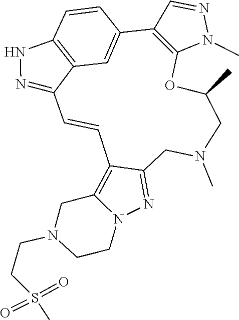 [SMILES: C[C@H]1CN(C)Cc2nn3c(c2/C=C/c2n[nH]c4ccc(cc24)-c2cnn(C)c2O1)CN(CCS(C)(=O)=O)CC3] | 551.1 | 1H NMR (400 MHz, DMSO- d6) δ = 12.90 (br s, 1H), 8.46 (s, 1H), 8.20 (d, J = 17.6 Hz, 1H), 7.85 (s, 1H), 7.67 (dd, J = 1.3, 8.8 Hz, 1H), 7.48 (d, J = 8.6 Hz, 1H), 6.71 (d, J = 17.5 Hz, 1H), 4.78 (br t, J = 6.2 Hz, 1H), 4.12-4.06 (m, 2H), 4.03 (s, 1H), 3.95 (br d, J = 7.1 Hz, 1H), 3.92 (br d, J = 4.6 Hz, 1H), 3.67 (s, 3H), 3.60 (br d, J = 12.9 Hz, 1H), 3.50 (br t, J = 6.9 Hz, 2H), 3.09 (br t, J = 7.1 Hz, 3H), 3.04 (s, 3H), 2.99- 2.94 (m, 1H), 2.94-2.89 (m, 1H), 2.76 (dd, J = 7.9, 14.5 Hz, 1H), 2.28 (s, 3H), 1.02 (d, J = 6.3 Hz, 3H) |
| 167 | 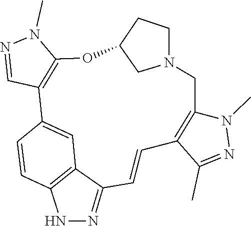 [SMILES: Cc1nn(C)c2c1/C=C/c1n[nH]c3ccc(cc13)-c1cnn(C)c1O[C@@H]1CCN(C2)C1] | 416.3 | 1H NMR (400 MHz, DMSO- d6) δ = 12.02 (s, 1H), 7.85 (s, 1H), 7.42 (d, J = 17.6 Hz, 1H), 7.05 (s, 1H), 6.81 (dd, J = 1.2, 8.7 Hz, 1H), 6.64 (d, J = 8.4 Hz, 1H), 6.09 (d, J = 17.6 Hz, 1H), 4.40 (br t, J = 5.2 Hz, 1H), 3.37 (d, J = 15.2 Hz, 1H), 2.97 (s, 3H), 2.89 (s, 3H), 2.81- 2.68 (m, 3H), 2.37-2.29 (m, 1H), 1.56 (s, 3H), 1.44-1.35 (m, 1H), 1.27-1.20 (m, 2H) |
| 168 | 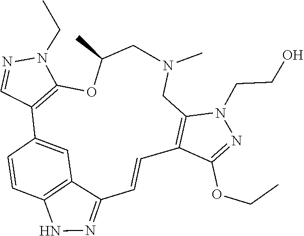 | 492.1 | 1H NMR (400 MHz, DMSO- d6) δ = 12.88-12.66 (m, 1H), 8.42 (s, 1H), 8.21-8.09 (m, 1H), 7.87 (s, 1H), 7.68-7.63 (m, 1H), 7.46 (d, J = 8.4 Hz, 1H), 7.14 (d, J = 17.2 Hz, 1H), 4.83 (t, J = 5.6 Hz, 1H), 4.80- 4.70 (m, 1H), 4.27 (ttd, J = 3.2, 7.0, 10.4 Hz, 2H), 4.07-3.97 (m, 4H), 3.97-3.91 (m, 1H), 3.85-3.78 (m, 1H), 3.69-3.59 (m, 2H), 2.99-2.91 (m, 1H), 2.85-2.74 (m, 1H), 2.35 (s, 3H), 1.44-1.38 (m, 3H), 1.38- 1.32 (m, 3H), 1.05 (d, J = 6.2 Hz, 3H) |
| 169 | 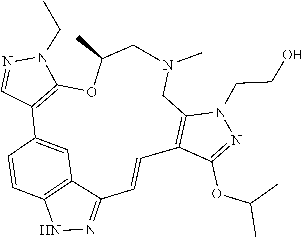 | 506.2 | 1H NMR (400 MHz, DMSO- d6) δ = 12.78 (s, 1H), 8.42 (s, 1H), 8.13 (d, J = 17.2 Hz, 1H), 7.87 (s, 1H), 7.69-7.58 (m, 1H), 7.46 (d, J = 8.8 Hz, 1H), 7.14 (d, J = 17.2 Hz, 1H), 4.95- 4.87 (m, 1H), 4.84 (t, J = 5.2 Hz, 1H), 4.80-4.72 (m, 1H), 4.07-3.97 (m, 4H), 3.95-3.77 (m, 2H), 3.69-3.60 (m, 2H), 2.98-2.91 (m, 1H), 2.78 (dd, J = 7.6, 14.0 Hz, 1H), 2.35 (s, 3H), 1.40 (d, J = 6.0 Hz, 3H), 1.38-1.32 (m, 6H), 1.04 (d, J = 6.4 Hz, 3H) |
| 170 | 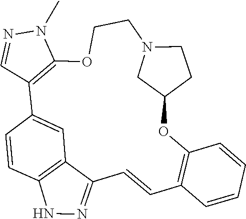 [SMILES: Cn1ncc2c1OCCN1CC[C@H](C1)Oc1ccccc1/C=C/c1n[nH]c3ccc-2cc13] | 428.2 | 1H NMR (400 MHz, METHANOL-d4) δ = 8.48- 8.31 (m, 1H), 7.88-7.73 (m, 2H), 7.70 (s, 1H), 7.61-7.53 (m, 2H), 7.48-7.37 (m, 1H), 7.36-7.30 (m, 1H), 7.13-7.01 (m, 2H), 5.43-5.25 (m, 1H), 4.56-4.38 (m, 2H), 4.30-4.07 (m, 2H), 4.02-3.88 (m, 2H), 3.85 (s, 3H), 3.78-3.58 (m, 2H), 2.66-2.36 (m, 2H) |
| 171 | 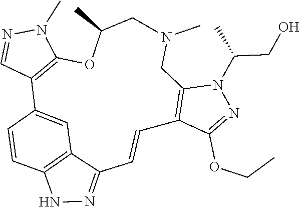 [SMILES: CCOc1nn([C@H](C)CO)c2c1/C=C/c1n[nH]c3ccc(cc13)-c1cnn(C)c1O[C@@H](C)CN(C)C2] | 492.2 | 1H NMR (400 MHz, DMSO- d6) δ = 12.80-12.77 (m, 1H), 8.50-8.46 (m, 1H), 8.20-8.12 (d, J = 17.2 Hz, 1H), 7.89- 7.86 (m, 1H), 7.70-7.65 (m, 1H), 7.49-7.44 (m, 1H), 7.19- 7.13 (d, J = 17.2 Hz, 1H), 4.86-4.70 (m, 2H), 4.41-4.34 (m, 1H), 4.29 (q, J = 7.2 Hz, 2H), 3.95-3.83 (m, 2H), 3.69- 3.67 (m, 3H), 3.67-3.53 (m, 2H), 2.99-2.93 (m, 1H), 2.82 (dd, J = 7.2, 14.4 Hz, 1H), 2.35 (s, 3H), 1.42 (t, J = 7.2 Hz, 3H), 1.29 (d, J = 6.8 Hz, 3H), 1.10-1.05 (m, 3H) |
| 172 | 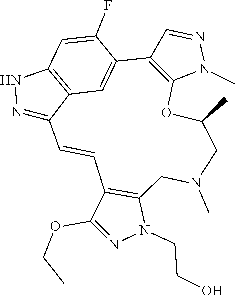 [SMILES: CCOc1nn(CCO)c2c1/C=C/c1n[nH]c3cc(F)c(cc13)-c1cnn(C)c1O[C@@H](C)CN(C)C2] | 496.3 | 1H NMR (400 MHz, METHANOL-d4) δ = 8.66 (d, J = 7.6 Hz, 1H), 7.87 (d, J = 4.8 Hz, 1H), 7.74-7.49 (m, 1H), 7.36 (d, J = 16.4 Hz, 1H), 7.30 (d, J = 11.6 Hz, 1H), 5.32- 5.11 (m, 1H), 4.43-4.36 (m, 2H), 4.22 (t, J = 5.2 Hz, 2H), 3.96-3.86 (m, 2H), 3.80 (s, 3H), 3.61-3.46 (m, 2H), 3.34- 3.32 (m, 3H), 3.01-2.88 (m, 2H), 1.50 (t, J = 7.2 Hz, 3H), 1.18 (d, J = 6.4 Hz, 3H) |
| 173 | 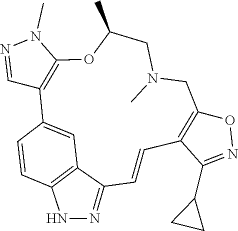 [SMILES: C[C@H]1CN(C)Cc2onc(C3CC3)c2/C=C/c2n[nH]c3ccc(cc23)-c2cnn(C)c2O1] | 431.0 | 1H NMR (400 MHz, DMSO- d6) δ = 8.49 (s, 1H), 8.21 (d, J = 17.6 Hz, 1H), 7.91 (s, 1H), 7.72 (dd, J = 0.9, 8.7 Hz, 1H), 7.52 (d, J = 8.7 Hz, 1H), 7.40 (d, J = 17.4 Hz, 1H), 4.74 (br t, J = 5.6 Hz, 1H), 4.14 (br d, J = 15.1 Hz, 1H), 3.90 (br d, J = 15.1 Hz, 1H), 3.68 (s, 3H), 3.00-2.84 (m, 2H), 2.36 (s, 3H), 2.23-2.14 (m, 1H), 1.14- 1.05 (m, 5H), 1.02-0.94 (m, 1H), 0.92-0.85 (m, 1H |
| 174 | 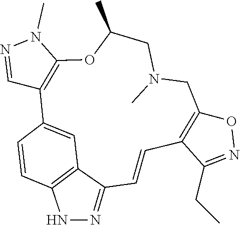 [SMILES: CCc1noc2c1/C=C/c1n[nH]c3ccc(cc13)-c1cnn(C)c1O[C@@H](C)CN(C)C2] | 418.8 | H NMR (400 MHz, DMSO- d6) δ = 8.47 (s, 1H), 8.18 (d, J = 17.8 Hz, 1H), 7.90 (s, 1H), 7.75-7.69 (m, 1H), 7.52 (d, J = 8.7 Hz, 1H), 7.07 (d, J = 17.6 Hz, 1H), 4.74 (br t, J = 5.6 Hz, 1H), 4.16 (d, J = 15.3 Hz, 1H), 3.92 (br d, J = 15.1 Hz, 1H), 3.68 (s, 3H), 3.01- 2.84 (m, 4H), 2.35 (s, 3H), 1.33 (t, J = 7.4 Hz, 3H), 1.07 (d, J = 6.2 Hz, 3H |
| 175 | 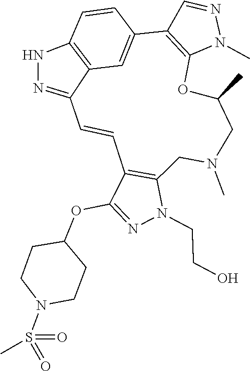 | 611.5 | 1H NMR (400 MHz, DMSO- d6) δ = 12.79 (s, 1H), 8.46 (s, 1H), 8.16 (d, J = 17.6 Hz, 1H), 7.87 (s, 1H), 7.72-7.59 (m, 1H), 7.52-7.41 (m, 1H), 7.18 (d, J = 17.2 Hz, 1H), 4.94- 4.80 (m, 2H), 4.79-4.70 (m, 1H), 4.10-3.98 (m, 2H), 3.97- 3.79 (m, 2H), 3.67 (s, 3H), 3.65-3.58 (m, 2H), 3.27-3.18 (m, 4H), 2.91 (s, 3H), 2.87- 2.76 (m, 2H), 2.39-2.31 (m, 3H), 2.18-2.06 (m, 2H), 1.97- 1.86 (m, 2H), 1.06 (br d, J = 6.4 Hz, 3H) |
| 176 | 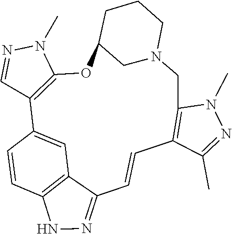 [SMILES: Cc1nn(C)c2c1/C=C/c1n[nH]c3ccc(cc13)-c1cnn(C)c1O[C@H]1CCCN(C2)C1] | 430.1 | 1H NMR (400 MHz, DMSO- d6) δ = 12.90-12.82 (m, 1H), 8.83-8.71 (m, 1H), 8.20-8.07 (m, 1H), 7.91 (s, 1H), 7.69 (d, J = 8.2 Hz, 1H), 7.46 (d, J = 8.8 Hz, 1H), 6.96 (d, J = 17.2 Hz, 1H), 4.97-4.90 (m, 1H), 4.06 (d, J = 15.2 Hz, 1H), 3.79 (s, 3H), 3.71 (s, 3H), 3.55 (d, J = 15.2 Hz, 1H), 2.40 (s, 3H), 2.15-1.93 (m, 2H), 1.88-1.69 (m, 2H), 1.68-1.56 (m, 2H), 1.41-1.23 (m, 2H) |
| 177 | 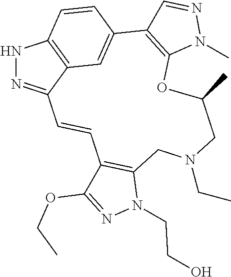 [SMILES: CCOc1nn(CCO)c2c1/C=C/c1n[nH]c3ccc(cc13)-c1cnn(C)c1O[C@@H](C)CN(CC)C2] | 492.2 | 1H NMR (400 MHz, METHANOL-d4) δ = 8.62 (s, 1H), 7.86 (s, 1H), 7.75-7.67 (m, 1H), 7.54 (d, J = 8.8 Hz, 1H), 7.50-7.37 (m, 2H), 5.44 (br s, 1H), 5.03-4.89 (m, 3H), 4.62 (br d, J = 15.2 Hz, 1H), 4.47-4.36 (m, 2H), 4.31-4.14 (m, 2H), 4.06-3.83 (m, 4H), 3.60 (br d, J = 5.6 Hz, 1H), 3.50 (br d, J = 14.0 Hz, 1H), 3.39 (br d, J = 5.2 Hz, 1H), 1.56-1.46 (m, 6H), 1.21 (d, J = 6.4 Hz, 3H) |
| 178 | 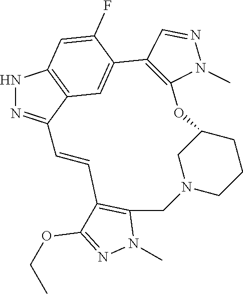 [SMILES: CCOc1nn(C)c2c1/C=C/c1n[nH]c3cc(F)c(cc13)-c1cnn(C)c1O[C@@H]1CCCN(C2)C1] | 478.1 | 1H NMR (400 MHz, METHANOL-d4) δ = 9.01- 8.90 (m, 1H), 8.30-8.19 (m, 1H), 7.88-7.83 (m, 1H), 7.45- 7.37 (m, 1H), 7.23-7.17 (m, 1H), 4.89-4.88 (m, 1H), 4.32 (d, J = 7.2 Hz, 2H), 3.98 (d, J = 15.6 Hz, 1H), 3.80 (s, 3H), 3.65 (s, 3H), 3.58 (d, J = 15.6 Hz, 1H), 2.94-2.75 (m, 2H), 2.11-1.87 (m, 2H), 1.75-1.55 (m, 2H), 1.50 (t, J = 7.2 Hz, 3H), 1.30 (br s, 2H) |
| 179 | 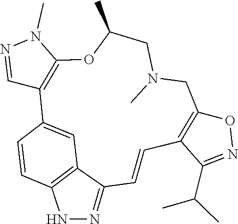 [SMILES: CC(C)c1noc2c1/C=C/c1n[nH]c3ccc(cc13)-c1cnn(C)c1O[C@@H](C)CN(C)C2] | 433.0 | 1H NMR (499 MHz, DMSO- d6) δ = 13.08 (s, 1H), 8.46 (s, 1H), 8.15 (d, J = 17.8 Hz, 1H), 7.89 (s, 1H), 7.72 (dd, J = 1.4, 8.8 Hz, 1H), 7.52 (d, J = 8.8 Hz, 1H), 7.08 (d, J = 17.8 Hz, 1H), 4.73 (br t, J = 5.7 Hz, 1H), 4.15 (d, J = 15.3 Hz, 1H), 3.90 (br d, J = 15.1 Hz, 1H), 3.68 (s, 3H), 3.41 (td, J = 6.8, 13.5 Hz, 1H), 2.98-2.83 (m, 2H), 2.34 (s, 3H), 1.39 (d, J = 6.8 Hz, 3H), 1.36 (d, J = 6.8 Hz, 3H), 1.06 (d, J = 6.3 Hz, 3H) |
| 180 | 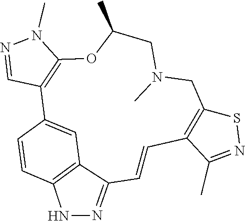 [SMILES: Cc1nsc2c1/C=C/c1n[nH]c3ccc(cc13)-c1cnn(C)c1O[C@@H](C)CN(C)C2] | 420.7 | 1H NMR (499 MHz, DMSO- d6) δ = 13.14 (s, 1H), 8.48 (s, 1H), 8.39 (d, J = 17.8 Hz, 1H), 7.86 (s, 1H), 7.70 (dd, J = 1.4, 8.8 Hz, 1H), 7.53 (d, J = 8.5 Hz, 1H), 7.22 (d, J = 17.5 Hz, 1H), 4.83 (br t, J = 6.3 Hz, 1H), 4.16 (d, J = 14.0 Hz, 1H), 3.86 (d, J = 13.7 Hz, 1H), 3.67 (s, 3H), 2.93-2.87 (m, 1H), 2.78 (dd, J = 8.5, 14.5 Hz, 1H), 2.69 (s, 3H), 2.30 (s, 3H), 1.00 (d, J = 6.3 Hz, 3H) |
| 181 | 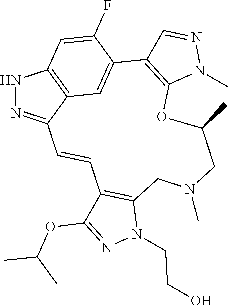 [SMILES: CC(C)Oc1nn(CCO)c2c1/C=C/c1n[nH]c3cc(F)c(cc13)-c1cnn(C)c1O[C@@H](C)CN(C)C2] | 510.2 | 1H NMR (400 MHz, DMSO- d6) δ = 8.54 (br d, J = 6.4 Hz, 1H), 8.06 (d, J = 17.2 Hz, 1H), 7.75 (d, J = 5.2 Hz, 1H), 7.34 (d, J = 12.0 Hz, 1H), 7.12 (d, J = 17.2 Hz, 1H), 4.95-4.87 (m, 1H), 4.85 (br t, J = 4.0 Hz, 1H), 4.73-4.64 (m, 1H), 4.11- 3.98 (m, 2H), 3.97-3.90 (m, 1H), 3.87-3.78 (m, 1H), 3.70 (s, 3H), 3.65 (br s, 2H), 2.99 (br d, J = 14.0 Hz, 1H), 2.79 (br dd, J = 6.8, 15.2 Hz, 2H), 2.30 (s, 3H), 1.38 (t, J = 6.4 Hz, 6H), 1.04 (d, J = 6.4 Hz, 3H) |
| 182 | 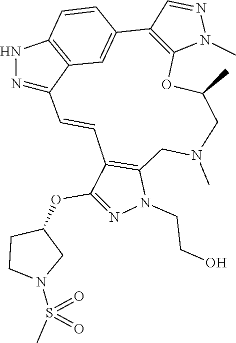 [SMILES: C[C@H]1CN(C)Cc2c(c(O[C@H]3CCN(S(C)(=O)=O)C3)nn2CCO)/C=C/c2n[nH]c3ccc(cc23)-c2cnn(C)c2O1] | 597.5 | 1H NMR (400 MHz, METHANOL-d4) δ = 8.58 (s, 1H), 7.87 (s, 1H), 7.75-7.68 (m, 1H), 7.61-7.49 (m, 2H), 7.35 (d, J = 16.4 Hz, 1H), 5.45- 5.40 (m, 1H), 5.35 (br d, J = 6.0 Hz, 1H), 4.91-4.89 (m, 1H), 4.55 (br d, J = 14.0 Hz, 1H), 4.26 (t, J = 4.8 Hz, 2H), 4.02-3.86 (m, 2H), 3.81-3.57 (m, 9H), 3.06 (s, 3H), 2.91 (s, 3H), 2.49-2.28 (m, 2H), 1.20 (d, J = 6.4 Hz, 3H) |
| 183 | 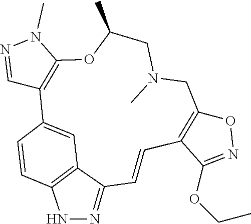 [SMILES: CCOc1noc2c1/C=C/c1n[nH]c3ccc(cc13)-c1cnn(C)c1O[C@@H](C)CN(C)C2] | 434.9 | 1H NMR (499 MHz, DMSO- d6) δ = 13.03 (s, 1H), 8.47 (s, 1H), 8.12 (d, J = 17.2 Hz, 1H), 7.91 (s, 1H), 7.72 (dd, J = 1.4, 8.8 Hz, 1H), 7.50 (d, J = 8.5 Hz, 1H), 7.30 (d, J = 17.2 Hz, 1H), 4.78-4.69 (m, 1H), 4.42- 4.34 (m, 2H), 4.10 (br d, J = 15.6 Hz, 1H), 3.87 (br d, J = 15.3 Hz, 1H), 3.68 (s, 3H), 3.04-2.97 (m, 1H), 2.88 (dd, J = 6.7, 14.4 Hz, 1H), 2.40 (s, 3H), 1.47 (t, J = 7.0 Hz, 3H), 1.09 (d, J = 6.3 Hz, 3H |
| 184 | 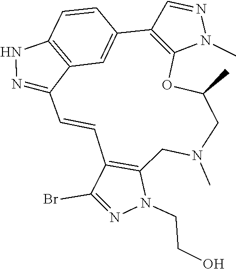 [SMILES: C[C@H]1CN(C)Cc2c(c(Br)nn2CCO)/C=C/c2n[nH]c3ccc(cc23)-c2cnn(C)c2O1] | 514.0 | 1H NMR (400 MHz, METHANOL-d4) δ = 8.57 (s, 1H), 7.92-7.83 (m, 2H), 7.79- 7.73 (m, 1H), 7.73-7.69 (m, 1H), 7.56 (d, J = 8.8 Hz, 1H), 5.22 (br s, 1H), 4.72-4.60 (m, 1H), 4.45 (br d, J = 14.4 Hz, 1H), 4.35 (t, J = 5.2 Hz, 2H), 4.02-3.87 (m, 2H), 3.77 (s, 3H), 3.54-3.38 (m, 2H), 2.85 (br s, 3H), 1.15 (d, J = 6.4 Hz, 3H) |
| 185 | 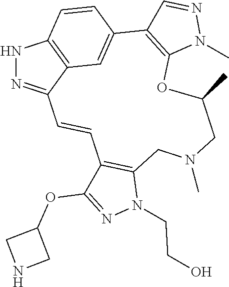 [SMILES: C[C@H]1CN(C)Cc2c(c(OC3CNC3)nn2CCO)/C=C/c2n[nH]c3ccc(cc23)-c2cnn(C)c2O1] | 505.2 | 1H NMR (400 MHz, DMSO- d6) δ = 13.34-12.49 (m, 1H), 8.92-8.75 (m, 1H), 8.46 (br s, 1H), 7.89 (s, 1H), 7.69 (br d, J = 8.8 Hz, 1H), 7.52-7.46 (m, 1H), 7.22 (br d, J = 16.8 Hz, 1H), 5.43-5.22 (m, 2H), 4.87- 4.67 (m, 2H), 4.51-4.34 (m, 3H), 4.23-3.97 (m, 6H), 3.81- 3.59 (m, 6H), 3.09-2.75 (m, 2H), 2.43-2.34 (m, 1H), 1.07 (br d, J = 6.0 Hz, 3H) |
| 186 | 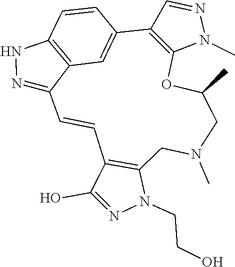 [SMILES: C[C@H]1CN(C)Cc2c(c(O)nn2CCO)/C=C/c2n[nH]c3ccc(cc23)-c2cnn(C)c2O1] | 450.2 | 1H NMR (400 MHz, METHANOL-d4) δ = 8.56 (s, 1H), 8.30 (d, J = 17.2 Hz, 1H), 7.83 (s, 1H), 7.70-7.63 (m, 1H), 7.49 (d, J = 8.8 Hz, 1H), 7.37-7.30 (m, 1H), 4.11-4.03 (m, 2H), 4.01-3.94 (m, 1H), 3.91-3.86 (m, 1H), 3.81 (t, J = 5.6 Hz, 2H), 3.74 (s, 3H), 3.43- 3.38 (m, 1H), 3.01-2.86 (m, 2H), 2.51-2.45 (m, 3H), 1.14- 1.10 (m, 3H) |
| 187 | 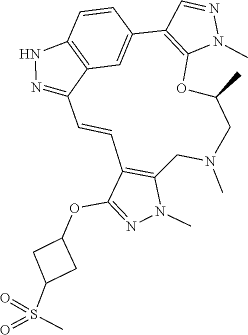 [SMILES: C[C@H]1CN(C)Cc2c(c(OC3CC(S(C)(=O)=O)C3)nn2C)/C=C/c2n[nH]c3ccc(cc23)-c2cnn(C)c2O1] | 552.1 | 1H NMR (400 MHz, MeOH- d4) δ = 8.57-8.53 (m, 1H), 8.22 (br d, J = 16.8 Hz, 1H), 7.83 (s, 1H), 7.69 (d, J = 9.2 Hz, 1H), 7.49 (br d, J = 9.2 Hz, 1H), 7.31-7.22 (br d, J = 16.8 Hz, 1H), 4.62-4.56 (m, 2H), 3.96 (br d, J = 14.4 Hz, 1H), 3.83-3.79 (m, 1H), 3.73 (d, J = 10.4 Hz, 6H), 2.94-2.89 (m, 6H), 2.69-2.61 (m, 2H), 2.43 (s, 3H), 1.30 (br s, 2H), 1.17- 1.11 (m, 3H). |
| 188 | 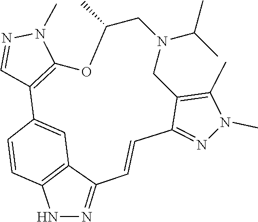 [SMILES: Cc1c2c(nn1C)/C=C/c1n[nH]c3ccc(cc13)-c1cnn(C)c1O[C@H](C)CN(C(C)C)C2] | 445.26 | 1H NMR (400 MHz, DMSO- d6) δ = 13.31-12.85 (m, 1H), 8.60 (s, 1H), 7.88 (s, 1H), 7.73- 7.66 (m, 1H), 7.54 (br d, J = 8.8 Hz, 1H), 7.48 (br d, J = 16.4 Hz, 1H), 7.34-7.26 (m, 1H), 5.49 (br d, J = 7.6 Hz, 1H), 4.81-4.71 (m, 1H), 4.33- 4.21 (m, 1H), 3.93-3.85 (m, 2H), 3.83 (s, 3H), 3.70 (s, 3H), 3.14-3.05 (m, 1H), 2.34 (s, 3H), 1.46 (br t, J = 5.2 Hz, 6H), 1.06 (br d, J = 6.0 Hz, 3H) |
| 189 | 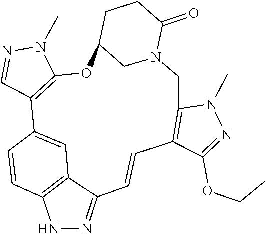 | 474.3 | 1H NMR (400 MHz, METHANOL-d4) δ = 8.34 (s, 1H), 7.83-7.74 (m, 1H), 7.66- 7.59 (m, 1H), 7.49 (d, J = 8.8 Hz, 1H), 7.35 (d, J = 16.4 Hz, 1H), 7.19-7.11 (m, 1H), 5.07- 4.95 (m, 2H), 4.35-4.26 (m, 3H), 4.16-3.96 (m, 2H), 3.82 (d, J = 2.8 Hz, 6H), 2.54-2.37 (m, 2H), 2.35-2.22 (m, 1H), 2.22-2.09 (m, 1H), 1.46 (t, J = 7.2 Hz, 3H) |
| 190 | 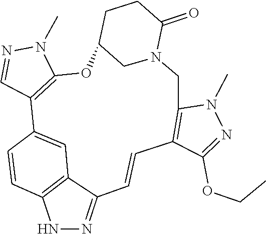 | 474.2 | 1H NMR (400 MHz, METHANOL-d4) δ = 8.33 (s, 1H), 7.79 (s, 1H), 7.66-7.59 (m, 1H), 7.49 (d, J = 8.8 Hz, 1H), 7.34 (d, J = 16.4 Hz, 1H), 7.20-7.08 (m, 1H), 5.07-4.95 (m, 2H), 4.38-4.25 (m, 3H), 4.15-3.96 (m, 2H), 3.82 (d, J = 3.6 Hz, 6H), 2.57-2.35 (m, 2H), 2.33-2.24 (m, 1H), 2.21- 2.09 (m, 1H), 1.46 (t, J = 7.2 Hz, 3H) |
| 191 | 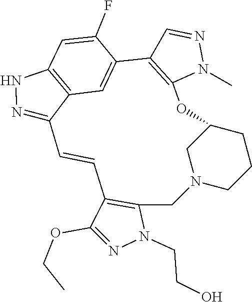 [SMILES: CCOc1nn(CCO)c2c1/C=C/c1n[nH]c3cc(F)c(cc13)-c1cnn(C)c1O[C@@H]1CCCN(C2)C1] | 508.1 | 1H NMR (400 MHz, DMSO- d6) δ = 12.80 (s, 1H), 8.99- 8.80 (m, 1H), 8.25-8.06 (m, 1H), 7.86-7.74 (m, 1H), 7.38- 7.20 (m, 2H), 4.91 (s, 2H), 4.28 (d, J = 7.2 Hz, 2H), 4.08- 3.92 (m, 3H), 3.82 (s, 3H), 3.71-3.59 (m, 3H), 2.06-1.88 (m, 2H), 1.85-1.68 (m, 2H), 1.65-1.52 (m, 2H), 1.42 (t, J = 6.8 Hz, 3H), 1.24 (d, J = 1.6 Hz, 2H) |
| 192 | 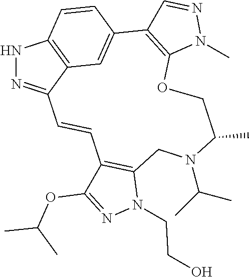 [SMILES: CC(C)Oc1nn(CCO)c2c1/C=C/c1n[nH]c3ccc(cc13)-c1cnn(C)c1OC[C@H](C)N(C(C)C)C2] | 520.4 | 1H NMR (499 MHz, METHANOL-d4) δ ppm 8.65 (s, 1 H), 7.86 (s, 1 H), 7.68 (dd, J = 8.76, 1.37 Hz, 1 H), 7.58-7.65 (m, 1 H), 7.48 (d, J = 8.76 Hz, 1 H), 7.35 (d, J = 16.70 Hz, 1 H), 5.00 (dt, J = 12.25, 6.06 Hz, 1 H), 4.54 (br d, J = 8.49 Hz, 1 H), 4.28- 4.35 (m, 1 H), 4.10 (dt, J = 14.30, 4.89 Hz, 1 H), 3.95 (s, 2 H), 3.84-3.94 (m, 4 H), 3.79 (s, 3 H), 3.41 (br dd, J = 12.05, 5.75 Hz, 1 H), 3.11- 3.18 (m, 1 H), 1.46 (dd, J = 6.02, 1.64 Hz, 6 H), 1.42 (d, J = 7.12 Hz, 3 H), 1.19 (br d, |
| | | | J = 6.30 Hz, 3 H), 1.04 (d, |
| | | | J = 6.84 Hz, 3 H) |
| 193 | 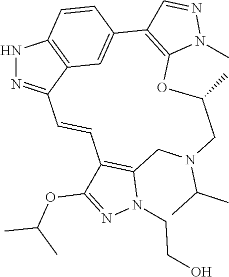 [SMILES: CC(C)Oc1nn(CCO)c2c1/C=C/c1n[nH]c3ccc(cc13)-c1cnn(C)c1O[C@H](C)CN(C(C)C)C2] | 520.4 | 1H NMR (499 MHz, METHANOL-d4) δ ppm 8.70 (br s, 1 H), 8.07 (br d, J = 16.70 Hz, 1 H), 7.83 (s, 1 H), 7.66 (dd, J = 8.76, 1.37 Hz, 1 H), 7.48 (d, J = 8.76 Hz, 1 H), 7.31 (d, J = 16.97 Hz, 1 H), 4.99 (dt, J = 12.25, 6.06 Hz, 1 H), 4.76 (br s, 1 H), 4.10-4.21 (m, 2 H), 3.98-4.08 (m, 2 H), 3.85 (t, J = 5.61 Hz, 2 H), 3.76 (s, 3 H), 2.89 (td, J = 13.00, 6.30 Hz, 3 H), 1.45 (dd, J = 6.02, 4.38 Hz, 6 H), 1.13 (d, J = 6.30 Hz, 3 H), 1.03 (d, J = 6.57 Hz, 3 H), 0.99 (d, J = 6.57 Hz, 3 H) |
| 194 | 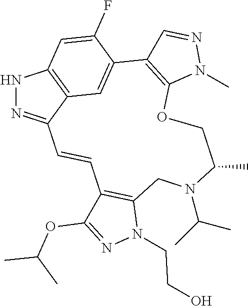 [SMILES: CC(C)Oc1nn(CCO)c2c1/C=C/c1n[nH]c3cc(F)c(cc13)-c1cnn(C)c1OC[C@H](C)N(C(C)C)C2] | 538.4 | 1H NMR (400 MHz, DMSO- d6) δ = 12.89 (s, 1H), 8.64 (br d, J = 7.2 Hz, 1H), 7.78 (d, J = 4.8 Hz, 1H), 7.52-7.43 (m, 1H), 7.34 (d, J = 12.4 Hz, 1H), 7.17 (d, J = 16.8 Hz, 1H), 4.96- 4.90 (m, 1H), 4.89 (br t, J = 5.2 Hz, 1H), 4.57 (br dd, J = 2.8, 5.2 Hz, 1H), 4.21-4.12 (m, 1H), 4.06-3.98 (m, 1H), 3.94-3.81 (m, 3H), 3.77 (s, 3H), 3.74-3.68 (m, 2H), 3.25 (br s, 1H), 3.17-2.98 (m, 1H), 1.40 (t, J = 5.6 Hz, 6H), 1.31 (d, J = 6.8 Hz, 3H), 1.12 (br d, J = 6.4 Hz, 3H), 0.95 (d, J = 6.4 Hz, 3H) |
| 195 | 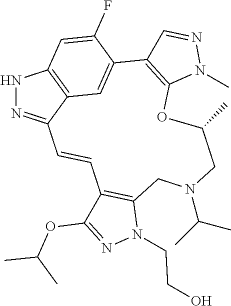 [SMILES: CC(C)Oc1nn(CCO)c2c1/C=C/c1n[nH]c3cc(F)c(cc13)-c1cnn(C)c1O[C@H](C)CN(C(C)C)C2] | 538.4 | 1H NMR (400 MHz, DMSO- d6) δ = 12.87 (s, 1H), 8.84- 8.60 (m, 1H), 7.87 (br d, J = 16.4 Hz, 1H), 7.75 (d, J = 5.2 Hz, 1H), 7.33 (d, J = 11.6 Hz, 1H), 7.12 (d, J = 17.2 Hz, 1H), 4.97-4.90 (m, 1H), 4.89-4.84 (m, 1H), 4.63- 4.46 (m, 1H), 4.05 (br t, J = 5.2 Hz, 2H), 4.01-3.90 (m, 2H), 3.73 (s, 3H), 3.70-3.63 (m, 3H), 2.83-2.73 (m, 2H), 1.40 (dd, J = 4.0, 6.0 Hz, 6H), 1.06 (br d, J = 6.0 Hz, 3H), 0.93 (br d, J = 4.4 Hz, 6H) |
| 196 | 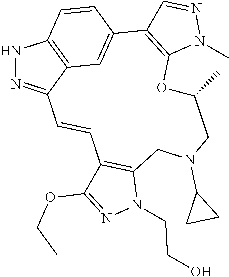 [SMILES: CCOc1nn(CCO)c2c1/C=C/c1n[nH]c3ccc(cc13)-c1cnn(C)c1O[C@H](C)CN(C1CC1)C2] | 504.2 | 1H NMR (400 MHz, DMSO- d6) δ = 12.80 (s, 1H), 8.43 (s, 1H), 7.88 (s, 1H), 7.65 (d, J = 8.8 Hz, 1H), 7.47 (d, J = 10.0 Hz, 1H), 7.44 (s, 1H), 7.12 (d, J = 16.8 Hz, 1H), 4.89-4.68 (m, 2H), 4.33-4.23 (m, 4H), 4.10-3.96 (m, 3H), 3.94-3.84 (m, 1H), 3.70 (d, J = 4.8 Hz, 2H), 3.68 (s, 3H), 2.10-2.00 (m, 1H), 1.41 (t, J = 7.2 Hz, 3H), 1.02 (d, J = 6.0 Hz, 3H), 0.42-0.28 (m, 3H), 0.12-0.02 (m, 1H) |
| 197 | 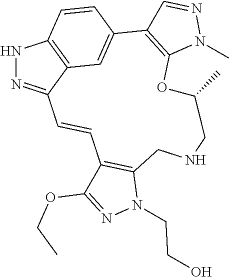 [SMILES: CCOc1nn(CCO)c2c1/C=C/c1n[nH]c3ccc(cc13)-c1cnn(C)c1O[C@H](C)CNC2] | 464.2 | 1H NMR (400 MHz, DMSO- d6) δ = 13.11-12.76 (m, 1H), 8.47 (s, 1H), 7.90 (s, 1H), 7.73- 7.62 (m, 1H), 7.51 (d, J = 8.8 Hz, 1H), 7.46-7.28 (m, 1H), 7.16 (d, J = 16.4 Hz, 1H), 5.31- 5.11 (m, 1H), 4.78-4.43 (m, 2H), 4.38-4.28 (m, 2H), 4.27- 4.09 (m, 2H), 3.72 (s, 3H), 3.69 (t, J = 4.8 Hz, 2H), 1.42 (t, J = 7.2 Hz, 3H), 1.12 (d, J = 6.4 Hz, 3H), 1.05 (t, J = 7.2 Hz, 2H) |
| 198 | 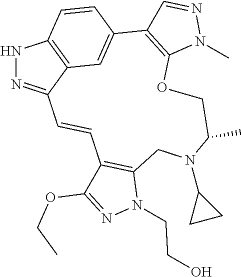 [SMILES: CCOc1nn(CCO)c2c1/C=C/c1n[nH]c3ccc(cc13)-c1cnn(C)c1OC[C@H](C)N(C1CC1)C2] | 504.1 | 1H NMR (400 MHz, DMSO- d6) δ = 12.82 (s, 1H), 8.49 (s, 1H), 7.88 (s, 1H), 7.64 (d, J = 8.8 Hz, 1H), 7.46 (d, J = 8.8 Hz, 1H), 7.36 (d, J = 16.8 Hz, 1H), 7.15 (d, J = 16.8 Hz, 1H), 4.91-4.83 (m, 1H), 4.64-4.55 (m, 1H), 4.32-4.25 (m, 2H), 4.22-4.13 (m, 1H), 4.11-4.04 (m, 1H), 4.03-3.96 (m, 2H), 3.88-3.80 (m, 1H), 3.74 (s, 3H), 3.72-3.67 (m, 2H), 2.15- 2.08 (m, 1H), 1.41 (t, J = 7.2 Hz, 3H), 1.32 (d, J = 6.8 Hz, 3H), 1.27-1.20 (m, 1H), 0.53- 0.11 (m, 4H) |
| 199 | 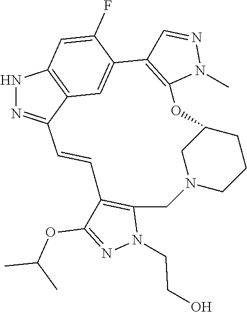 [SMILES: CC(C)Oc1nn(CCO)c2c1/C=C/c1n[nH]c3cc(F)c(cc13)-c1cnn(C)c1O[C@@H]1CCCN(C2)C1] | 522.2 | 1H NMR (400 MHz, METHANOL-d4) δ = 9.04- 8.90 (m, 1H), 8.37-8.14 (m, 1H), 7.92 (d, J = 4.8 Hz, 1H), 7.46 (d, J = 17.2 Hz, 1H), 7.25 (d, J = 12.4 Hz, 1H), 5.02- 4.99 (m, 1H), 4.11-4.04 (m, 3H), 3.88-3.84 (m, 5H), 3.72 (d, J = 15.2 Hz, 1H), 3.52- 3.49 (m, 1H), 3.01-2.81 (m, 2H), 1.98-1.59 (m, 4H), 1.47 (d, J = 6.0 Hz, 6H), 1.38-1.31 (m, 2H) |
| 200 | 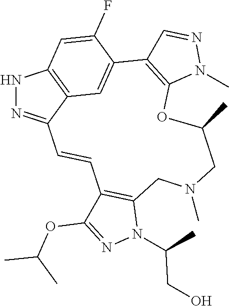 [SMILES: CC(C)Oc1nn([C@@H](C)CO)c2c1/C=C/c1n[nH]c3cc(F)c(cc13)-c1cnn(C)c1O[C@@H](C)CN(C)C2] | 524.2 | 1H NMR (400 MHz, METHANOL-d4) δ = 8.63 (d, J = 7.2 Hz, 1H), 7.86 (d, J = 4.8 Hz, 1H), 7.53-7.42 (m, 1H), 7.41-7.34 (m, 1H), 7.31 (d, J = 12.0 Hz, 1H), 5.31 (d, J = 1.6 Hz, 1H), 5.13-5.04 (m, 1H), 4.85-4.80 (m, 2H), 4.61- 4.48 (m, 2H), 3.80 (s, 3H), 3.78-3.59 (m, 4H), 3.02 (s, 3H), 1.53-1.48 (m, 3H), 1.48- 1.43 (m, 6H), 1.19 (d, J = 6.4 Hz, 3H) |
| 201 | 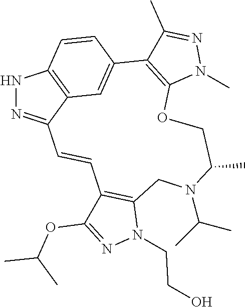 [SMILES: Cc1nn(C)c2c1-c1ccc3[nH]nc(c3c1)/C=C/c1c(OC(C)C)nn(CCO)c1CN(C(C)C)[C@@H](C)CO2] | 533.31 | 1H NMR (400 MHz, DMSO- d6) δ = 12.79 (br s, 1H), 8.51 (s, 1H), 7.62-7.54 (m, 2H), 7.52-7.47 (m, 1H), 7.17 (d, J = 16.8 Hz, 1H), 4.95-4.88 (m, 1H), 4.40-4.31 (m, 1H), 4.17- 4.07 (m, 1H), 4.05-3.96 (m, 1H), 3.95-3.84 (m, 3H), 3.72- 3.68 (m, 2H), 3.67 (s, 3H), 3.28-3.20 (m, 2H), 3.00-2.90 (m, 1H), 2.39 (s, 3H), 1.39 (d, J = 6.0 Hz, 6H), 1.32 (d, J = 6.8 Hz, 3H), 1.05 (br d, J = 6.4 Hz, 3H), 0.89 (d, J = 6.4 Hz, 3H) |
| 202 | 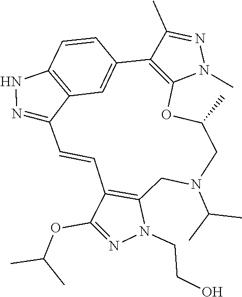 [SMILES: Cc1nn(C)c2c1-c1ccc3[nH]nc(c3c1)/C=C/c1c(OC(C)C)nn(CCO)c1CN(C(C)C)C[C@@H](C)O2] | 534.4 | 1H NMR (400 MHz, DMSO- d6) δ = 12.77 (br s, 1H), 8.46 (s, 1H), 7.87 (br d, J = 17.2 Hz, 1H), 7.55-7.51 (m, 1H), 7.51- 7.47 (m, 1H), 7.09 (d, J = 17.2 Hz, 1H), 4.95-4.85 (m, 1H), 4.44 (br s, 1H), 4.03 (br t, J = 5.6 Hz, 2H), 3.98 (s, 2H), 3.66 (br t, J = 5.6 Hz, 2H), 3.63 (s, 3H), 2.85-2.62 (m, 4H), 2.34 (s, 3H), 1.41-1.37 (m, 6H), 0.94 (br d, J = 6.4 Hz, 3H), 0.85 (br t, J = 6.8 Hz, 6H |
| 203 | 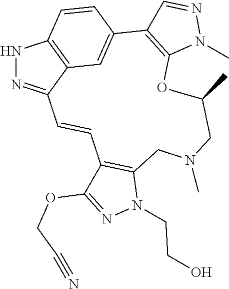 [SMILES: C[C@H]1CN(C)Cc2c(c(OCC#N)nn2CCO)/C=C/c2n[nH]c3ccc(cc23)-c2cnn(C)c2O1] | 489.3 | 1H NMR (400 MHz, METHANOL-d4) δ = 8.55 (s, 1H), 8.37-8.30 (m, 1H), 7.88- 7.81 (m, 1H), 7.72-7.66 (m, 1H), 7.53-7.47 (m, 1H), 7.21 (d, J = 17.2 Hz, 1H), 5.18- 5.13 (m, 2H), 4.95-4.92 (m, 1H), 4.23-4.07 (m, 2H), 4.03- 3.92 (m, 2H), 3.91-3.85 (m, 2H), 3.74 (s, 3H), 3.02-2.86 (m, 2H), 2.50-2.44 (m, 3H), 1.17-1.07 (m, 3H) |
| 204 | 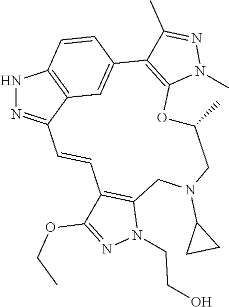 [SMILES: CCOc1nn(CCO)c2c1/C=C/c1n[nH]c3ccc(cc13)-c1c(C)nn(C)c1O[C@H](C)CN(C1CC1)C2] | 518.3 | 1H NMR (400 MHz, DMSO- d6) δ = 12.79 (s, 1H), 8.38 (s, 1H), 7.55-7.48 (m, 2H), 7.47- 7.42 (m, 1H), 7.06 (d, J = 16.8 Hz, 1H), 4.86 (br s, 1H), 4.57-4.50 (m, 1H), 4.32-4.21 (m, 3H), 4.09-3.95 (m, 3H), 3.73-3.68 (m, 2H), 3.63 (s, 3H), 3.12-3.06 (m, 1H), 3.05- 2.98 (m, 1H), 2.35 (s, 3H), 2.03-1.98 (m, 1H), 1.41 (t, J = 7.2 Hz, 3H), 0.89 (d, J = 6.4 Hz, 3H), 0.31-0.23 (m, 2H), 0.18 (br d, J = 11.6 Hz, 1H), −0.03 (br d, J = 6.0 Hz, 1H) |
| 205 | 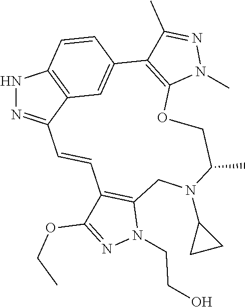 [SMILES: CCOc1nn(CCO)c2c1/C=C/c1n[nH]c3ccc(cc13)-c1c(C)nn(C)c1OC[C@H](C)N(C1CC1)C2] | 518.4 | 1H NMR (400 MHz, DMSO- d6) δ = 12.83 (s, 1H), 8.37 (s, 1H), 7.56-7.48 (m, 2H), 7.34 (d, J = 16.8 Hz, 1H), 7.12 (d, J = 16.8 Hz, 1H), 4.89-4.84 (m, 1H), 4.41-4.35 (m, 1H), 4.31- 4.25 (m, 2H), 4.20-3.97 (m, 5H), 3.84 (br d, J = 14.4 Hz, 1H), 3.72-3.70 (m, 1H), 3.68 (s, 3H), 3.37 (br d, J = 5.6 Hz, 1H), 2.35 (s, 3H), 2.13-2.06 (m, 1H), 1.41 (t, J = 7.2 Hz, 3H), 1.34 (d, J = 6.8 Hz, 3H), 0.41-0.29 (m, 2H), 0.20-0.04 (m, 2H) |
| 206 | 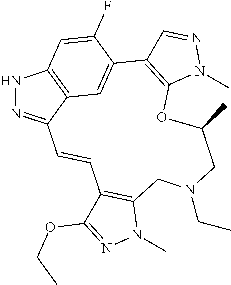 [SMILES: CCOc1nn(C)c2c1/C=C/c1n[nH]c3cc(F)c(cc13)-c1cnn(C)c1O[C@@H](C)CN(CC)C2] | 480.2 | 1H NMR (400 MHz, METHANOL-d4) δ = 8.71 (br d, J = 7.2 Hz, 1H), 8.03 (d, J = 17.2 Hz, 1H), 7.84 (d, J = 4.8 Hz, 1H), 7.27 (s, 1H), 7.23 (d, J = 3.6 Hz, 1H), 4.37-4.27 (m, 2H), 4.04-3.90 (m, 2H), 3.78 (br s, 1H), 3.77 (s, 3H), 3.74 (s, 3H), 3.06-2.95 (m, 2H), 2.68-2.55 (m, 2H), 1.49 (t, J = 7.2 Hz, 3H), 1.12 (d, J = 6.4 Hz, 3H), 1.00 (t, J = 7.2 Hz, 3H) |
| 207 | 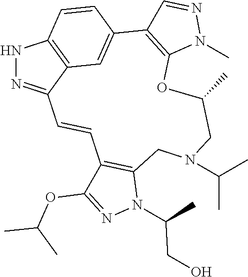 [SMILES: CC(C)Oc1nn([C@@H](C)CO)c2c1/C=C/c1n[nH]c3ccc(cc13)-c1cnn(C)c1O[C@H](C)CN(C(C)C)C2] | 534.3 | 1H NMR (400 MHz, DMSO- d6) δ 12.86 (s, 1H), 8.47 (s, 1H), 7.64 (s, 1H), 7.43 (d, J = 8.4 Hz, 1H), 7.28 (d, J = 8.4 Hz, 1H), 7.04 (s, 1H), 5.80 (s, 1H), 4.99 (s, 1H), 4.95-4.81 (m, 2H), 4.35-4.29 (m, 1H), 3.88-3.76 (m, 3H), 3.65 (s, 3H), 3.10-2.99 (m, 1H), 2.82 (dd, J = 5.2, 15.2 Hz, 1H), 2.65- 2.55 (m, 2H), 1.32 (d, J = 6.4 Hz, 6H), 1.19 (d, J = 5.6 Hz, 3H), 1.05 (d, J = 6.4 Hz, 3H), 0.92-0.86 (m, 3H), 0.51-0.21 (m, 3H) |
| 208 | 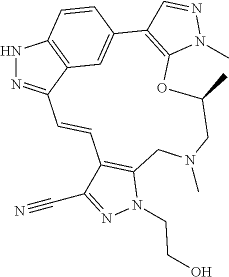 [SMILES: C[C@H]1CN(C)Cc2c(c(C#N)nn2CCO)/C=C/c2n[nH]c3ccc(cc23)-c2cnn(C)c2O1] | 459.3 | 1H NMR (400 MHz, DMSO- d6) δ = 13.23-13.01 (m, 1H), 8.47 (s, 1H), 8.33 (br d, J = 17.2 Hz, 1H), 7.90 (s, 1H), 7.72 (d, J = 8.8 Hz, 1H), 7.53 (d, J = 8.8 Hz, 1H), 7.46 (d, J = 17.6 Hz, 1H), 4.85-4.72 (m, 1H), 4.31 (br d, J = 3.6 Hz, 2H), 4.04 (br d, J = 14.4 Hz, 3H), 3.74 (br d, J = 4.4 Hz, 2H), 3.68 (s, 3H), 3.02-2.80 (m, 2H), 2.34 (br s, 3H), 1.06 (br d, J = 6.4 Hz, 3H) |
| 209 | 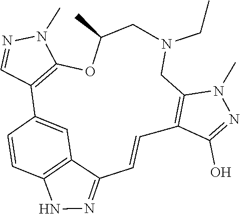 [SMILES: CCN1Cc2c(c(O)nn2C)/C=C/c2n[nH]c3ccc(cc23)-c2cnn(C)c2O[C@@H](C)C1] | 434.1 | 1H NMR (400 MHz, DMSO- d6) δ = 12.82-12.67 (m, 1H), 10.33 (d, J = 4.4 Hz, 1H), 8.56 (s, 1H), 7.97-7.85 (m, 2H), 7.69-7.61 (m, 1H), 7.45 (d, J = 8.8 Hz, 1H), 7.22-7.09 (m, 1H), 4.80-4.69 (m, 1H), 3.97- 3.89 (m, 1H), 3.84-3.78 (m, 1H), 3.68 (s, 3H), 3.63 (s, 3H), 2.95-2.90 (m, 2H), 2.63-2.59 (m, 2H), 1.08 (d, J = 6.0 Hz, 3H), 0.99-0.92 (m, 3H) |
| 210 | 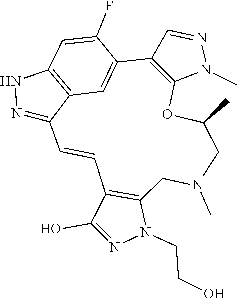 [SMILES: C[C@H]1CN(C)Cc2c(c(O)nn2CCO)/C=C/c2n[nH]c3cc(F)c(cc23)-c2cnn(C)c2O1] | 468.2 | 1H NMR (400 MHz, METHANOL-d4) δ = 8.68- 8.63 (m, 1H), 8.18-8.09 (m, 1H), 7.87-7.83 (m, 1H), 7.28 (s, 1H), 7.27-7.23 (m, 1H), 4.82-4.73 (m, 1H), 4.38-4.33 (m, 2H), 4.01-3.95 (m, 3H), 3.77-3.75 (m, 3H), 3.70 (s, 1H), 3.06-2.98 (m, 1H), 2.90- 2.82 (m, 1H), 2.41-2.36 (m, 3H), 1.16-1.11 (m, 3H) |
| 211 | 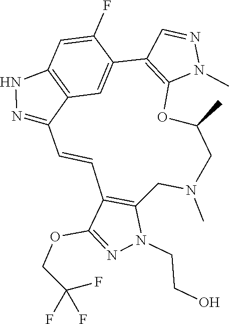 [SMILES: C[C@H]1CN(C)Cc2c(c(OCC(F)(F)F)nn2CCO)/C=C/c2n[nH]c3cc(F)c(cc23)-c2cnn(C)c2O1] | 550.2 | 1H NMR (400 MHz, METHANOL-d4) δ = 8.68 (br d, J = 7.2 Hz, 1H), 8.52 (br s, 1H), 8.17 (d, J = 17.2 Hz, 1H), 7.86 (d, J = 5.2 Hz, 1H), 7.46 (d, J = 11.6 Hz, 1H), 7.29 (d, J = 17.2 Hz, 1H), 5.15-5.07 (m, 2H), 4.59 (s, 2H), 4.38-4.33 (m, 2H), 4.02-3.95 (m, 3H), 3.77 (s, 3H), 3.05-2.99 (m, 1H), 2.89-2.81 (m, 1H), 2.38 (s, 3H), 1.13 (d, J = 6.4 Hz, 3H) |
| 212 | 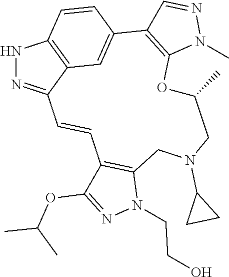 [SMILES: CC(C)Oc1nn(CCO)c2c1/C=C/c1n[nH]c3ccc(cc13)-c1cnn(C)c1O[C@H](C)CN(C1CC1)C2] | 518.1 | 1H NMR (400 MHz, DMSO- d6) δ = 12.80 (s, 1H), 8.43 (s, 1H), 7.87 (s, 1H), 7.65 (d, J = 7.6 Hz, 1H), 7.48-7.40 (m, 2H), 7.12 (d, J = 16.8 Hz, 1H), 4.96-4.89 (m, 1H), 4.85 (t, J = 5.2 Hz, 1H), 4.77-4.69 (m, 1H), 4.31-4.22 (m, 1H), 4.08- 3.99 (m, 2H), 3.94-3.84 (m, 1H), 3.72-3.68 (m, 2H), 3.68 (s, 3H), 3.19-3.12 (m, 1H), 3.06-2.97 (m, 1H), 1.39 (t, J = 6.0 Hz, 6H), 1.02 (d, J = 6.4 Hz, 3H), 0.41-0.27 (m, 3H), 0.13-0.02 (m, 1H) |
| 213 | 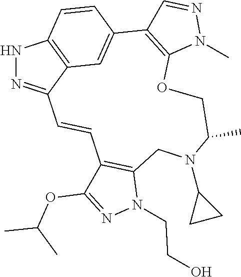 [SMILES: CC(C)Oc1nn(CCO)c2c1/C=C/c1n[nH]c3ccc(cc13)-c1cnn(C)c1OC[C@H](C)N(C1CC1)C2] | 518.2 | 1H NMR (400 MHz, DMSO- d6) δ = 12.83 (s, 1H), 8.49 (s, 1H), 7.89 (s, 1H), 7.70-7.63 (m, 1H), 7.47 (d, J = 8.8 Hz, 1H), 7.36 (d, J = 16.4 Hz, 1H), 7.22-7.12 (m, 1H), 4.98-4.91 (m, 1H), 4.90-4.87 (m, 1H), 4.70-4.49 (m, 1H), 4.24-4.13 (m, 1H), 4.12-3.91 (m, 4H), 3.75 (s, 3H), 3.72-3.68 (m, 2H), 2.16-2.08 (m, 2H), 1.39 (dd, J = 4.4, 6.0 Hz, 6H), 1.33 (br d, J = 6.8 Hz, 3H), 0.53- 0.29 (m, 4H) |
| 214 | 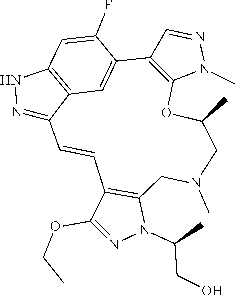 [SMILES: CCOc1nn([C@@H](C)CO)c2c1/C=C/c1n[nH]c3cc(F)c(cc13)-c1cnn(C)c1O[C@@H](C)CN(C)C2] | 510.2 | 1H NMR (400 MHz, METHANOL-d4) δ = 8.63 (d, J = 7.4 Hz, 1H), 7.91-7.83 (m, 1H), 7.68-7.51 (m, 1H), 7.40-7.26 (m, 2H), 5.34-5.14 (m, 1H), 5.02 (t, J = 5.1 Hz, 1H), 4.84-4.61 (m, 1H), 4.56- 4.49 (m, 1H), 4.45-4.38 (m, 2H), 3.80 (s, 3H), 3.73-3.67 (m, 1H), 3.55 (br t, J = 6.4 Hz, 1H), 2.92 (br s, 2H), 1.53- 1.45 (m, 6H), 1.19-1.17 (m, 6H) |
| 215 | 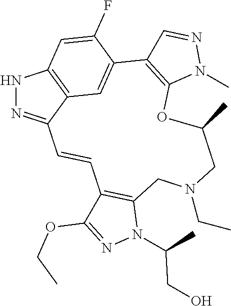 [SMILES: CCOc1nn([C@@H](C)CO)c2c1/C=C/c1n[nH]c3cc(F)c(cc13)-c1cnn(C)c1O[C@@H](C)CN(CC)C2] | 524.2 | 1H NMR (400 MHz, METHANOL-d4) δ = 8.74- 8.68 (m, 1H), 8.06 (d, J = 17.0 Hz, 1H), 7.84 (d, J = 4.8 Hz, 1H), 7.29-7.25 (m, 1H), 7.23 (s, 1H), 4.78-4.72 (m, 1H), 4.50 (br dd, J = 7.1, 12.0 Hz, 1H), 4.39-4.33 (m, 2H), 4.00 (s, 2H), 3.87-3.80 (m, 1H), 3.77 (s, 3H), 3.68 (dd, J = 4.7, 11.3 Hz, 1H), 3.08- 3.02 (m, 1H), 2.96-2.91 (m, 1H), 2.71-2.61 (m, 2H), 1.48 (t, J = 7.1 Hz, 3H), 1.42 (d, J = 6.6 Hz, 3H), 1.11 (d, J = 6.4 Hz, 3H), 1.01 (t, J = 7.0 Hz, 3H) |
| 216 | 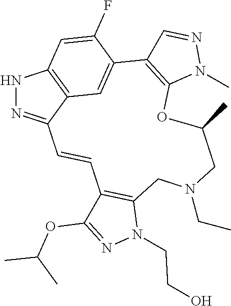 [SMILES: CCN1Cc2c(c(OC(C)C)nn2CCO)/C=C/c2n[nH]c3cc(F)c(cc23)-c2cnn(C)c2O[C@@H](C)C1] | 524.3 | 1H NMR (400 MHz, METHANOL-d4) δ = 8.82- 8.67 (m, 1H), 8.20-8.06 (m, 1H), 7.90-7.81 (m, 1H), 7.36- 7.19 (m, 2H), 4.62 (br s, 2H), 4.15 (br dd, J = 6.1, 11.7 Hz, 2H), 4.01 (br s, 2H), 3.86- 3.82 (m, 2H), 3.77 (s, 3H), 3.04-2.96 (m, 2H), 2.69-2.61 (m, 2H), 1.51-1.43 (m, 6H), 1.12 (br d, J = 6.1 Hz, 3H), 1.04-0.96 (m, 3H) |
| 217 | 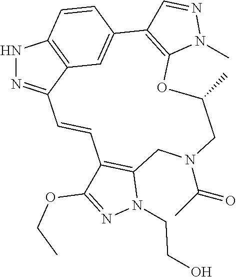 [SMILES: CCOc1nn(CCO)c2c1/C=C/c1n[nH]c3ccc(cc13)-c1cnn(C)c1O[C@H](C)CN(C(C)=O)C2] | 506.3 | 1H NMR (499 MHz, DMSO- d6) δ ppm 12.74-13.11 (m, 1 H) 8.44-8.61 (m, 1 H) 7.98 (s, 1 H) 7.70 (d, J = 9.03 Hz, 1 H) 7.46-7.50 (m, 1 H) 7.38-7.45 (m, 1 H) 7.22-7.29 (m, 1 H) 5.24-5.40 (m, 1 H) 5.23-5.39 (m, 1 H) 5.12-5.23 (m, 1 H) 5.12-5.21 (m, 1 H) 4.26-4.36 (m, 3 H) 4.19-4.25 (m, 1 H) 4.01-4.02 (m, 1 H) 4.00-4.09 (m, 1 H) 3.73 (br s, 10 H) 2.22 (s, 3 H) 1.42 (t, J = 7.12 Hz, 3 H) 1.19-1.29 (m, 3 H) 1.09- 1.18 (m, 3 H) |
| 218 | 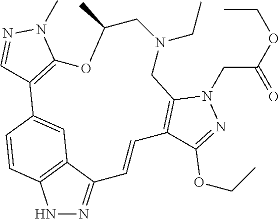 [SMILES: CCOC(=O)Cn1nc(OCC)c2c1CN(CC)C[C@H](C)Oc1c(cnn1C)-c1ccc3[nH]nc(c3c1)/C=C/2] | 534.3 | 1H NMR (400 MHz, DMSO- d6) δ = 12.86-12.81 (m, 1H), 8.60-8.54 (m, 1H), 8.10-8.03 (m, 1H), 7.88 (s, 1H), 7.70- 7.63 (m, 1H), 7.49-7.44 (m, 1H), 7.23-7.14 (m, 1H), 4.95 (s, 2H), 4.76-4.68 (m, 1H), 4.29-4.22 (m, 2H), 4.18-4.12 (m, 2H), 3.94-3.80 (m, 2H), 3.68 (s, 2H), 2.95-2.88 (m, 2H), 2.71-2.61 (m, 3H), 1.41 (s, 3H), 1.22 (s, 3H), 1.10- 1.05 (m, 3H), 0.93 (s, 3H) |
| 219 | 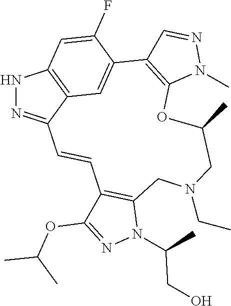 [SMILES: CCN1Cc2c(c(OC(C)C)nn2[C@@H](C)CO)/C=C/c2n[nH]c3cc(F)c(cc23)-c2cnn(C)c2O[C@@H](C)C1] | 538.3 | 1H NMR (400 MHz, METHANOL-d4) δ = 8.69 (br d, J = 7.2 Hz, 1H), 8.03 (d, J = 16.8 Hz, 1H), 7.83 (d, J = 4.8 Hz, 1H), 7.27 (d, J = 11.0 Hz, 1H), 7.23 (d, J = 6.0 Hz, 1H), 5.05-4.98 (m, 1H), 4.59 (s, 1H), 4.53-4.43 (m, 1H), 3.99 (s, 2H), 3.78- 3.76 (m, 3H), 3.71-3.66 (m, 1H), 3.08-3.00 (m, 1H), 2.95- 2.87 (m, 1H), 2.71-2.58 (m, 2H), 2.03 (s, 2H), 1.46-1.40 (m, 9H), 1.10 (d, J = 6.4 Hz, 3H), 1.00 (t, J = 7.2 Hz, 3H) |
| 220 | 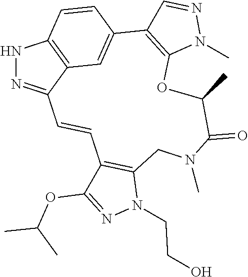 [SMILES: CC(C)Oc1nn(CCO)c2c1/C=C/c1n[nH]c3ccc(cc13)-c1cnn(C)c1O[C@@H](C)C(=O)N(C)C2] | 506.2 | 1H NMR (400 MHz, DMSO- d6) δ = 12.91 (s, 1H), 8.03- 7.82 (m, 2H), 7.67 (d, J = 8.8 Hz, 1H), 7.49 (d, J = 8.4 Hz, 1H), 7.34-7.17 (m, 1H), 6.92 (d, J = 16.8 Hz, 1H), 5.01- 4.83 (m, 2H), 4.06 (s, 2H), 3.76 (s, 3H), 3.69 (s, 2H), 3.33- 3.32 (m, 3H), 3.08 (s, 2H), 1.44 (s, 3H), 1.41-1.36 (m, 6H) |
| 221 | 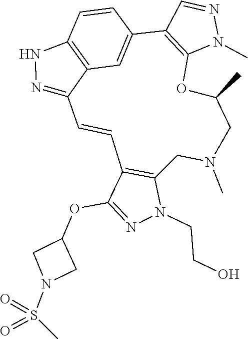 [SMILES: C[C@H]1CN(C)Cc2c(c(OC3CN(S(C)(=O)=O)C3)nn2CCO)/C=C/c2n[nH]c3ccc(cc23)-c2cnn(C)c2O1] | 583.3 | 1H NMR (400 MHz, METHANOL-d4) δ = 8.60- 8.51 (m, 1H), 8.40-8.23 (m, 1H), 7.93-7.77 (m, 1H), 7.75- 7.64 (m, 1H), 7.50 (br dd, J = 4.0, 8.7 Hz, 1H), 7.36-7.23 (m, 1H), 5.40-5.20 (m, 1H), 4.66-4.53 (m, 1H), 4.37 (br d, J = 4.0 Hz, 2H), 4.20-4.04 (m, 4H), 4.01-3.88 (m, 2H), 3.87-3.79 (m, 2H), 3.78-3.68 (m, 3H), 3.07-2.99 (m, 3H), 2.98-2.84 (m, 2H), 2.45 (br d, J = 4.0 Hz, 3H), 1.21-1.03 (m, 3H) |
| 222 | 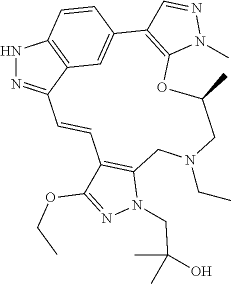 [SMILES: CCOc1nn(CC(C)(C)O)c2c1/C=C/c1n[nH]c3ccc(cc13)-c1cnn(C)c1O[C@@H](C)CN(CC)C2] | 520.2 | 1H NMR (400 MHz, METHANOL-d4) δ = 8.67- 8.63 (m, 1H), 8.23 (d, J = 17.0 Hz, 1H), 7.84 (s, 1H), 7.67 (d, J = 1.5 Hz, 1H), 7.49 (d, J = 8.5 Hz, 1H), 7.34 (d, J = 17.0 Hz, 1H), 4.96-4.90 (m, 6H), 4.36-4.29 (m, 2H), 3.92 (s, 3H), 3.76 (d, J = 10.9 Hz, 6H), 3.08-3.00 (m, 1H), 2.94-2.87 (m, 1H), 2.77-2.67 (m, 2H), 1.49 (t, J = 7.1 Hz, 3H), 1.13 (d, J = 6.4 Hz, 3H), 1.00 (t, J = 7.1 Hz, 3H) |
| 223 | 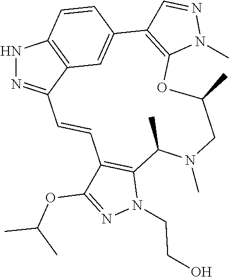 [SMILES: CC(C)Oc1nn(CCO)c2c1/C=C/c1n[nH]c3ccc(cc13)-c1cnn(C)c1O[C@@H](C)CN(C)[C@@H]2C] | 506.4 | 1H NMR (400 MHz, DMSO- d6) δ = 12.76 (s, 1H), 8.69 (s, 1H), 8.35 (d, J = 17.2 Hz, 1H), 7.89 (s, 1H), 7.67 (d, J = 9.2 Hz, 1H), 7.50-7.39 (m, 1H), 7.24-7.08 (m, 1H), 4.82 (s, 2H), 4.51-4.36 (m, 1H), 4.09 (s, 2H), 3.92-3.84 (m, 1H), 3.70 (s, 3H), 3.25-3.15 (m, 3H), 2.77-2.69 (m, 1H), 2.35 (s, 3H), 1.43 (d, J = 6.4 Hz., 3H), 1.39 (t, J = 6.4 Hz, 6H), 1.20 (d, J = 6.4 Hz, 3H) |
| 224 | 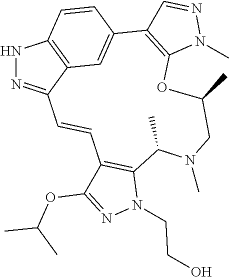 [SMILES: CC(C)Oc1nn(CCO)c2c1/C=C/c1n[nH]c3ccc(cc13)-c1cnn(C)c1O[C@@H](C)CN(C)[C@H]2C] | 506.2 | 1H NMR (400 MHz, DMSO- d6) δ = 12.78 (s, 1H), 8.61- 8.41 (m, 1H), 8.31 (d, J = 17.2 Hz, 1H), 7.86 (s, 1H), 7.66 (d, J = 8.4 Hz, 1H), 7.45 (d, J = 8.8 Hz, 1H), 7.08 (d, J = 17.2 Hz, 1H), 4.95-4.82 (m, 2H), 4.66-4.54 (m, 1H), 4.14-3.92 (m, 2H), 3.89- 3.81 (m, 1H), 3.67 (s, 3H), 2.56 (d, J = 7.6 Hz, 4H), 2.41 (s, 3H), 1.44-1.34 (m, 9H), 1.06 (d, J = 6.4 Hz, 3H) |
| 225 | 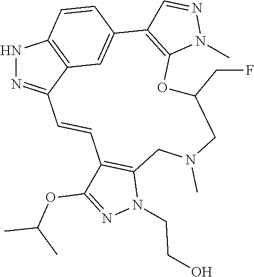 [SMILES: CC(C)Oc1nn(CCO)c2c1/C=C/c1n[nH]c3ccc(cc13)-c1cnn(C)c1OC(CF)CN(C)C2] | 510.2 | 1H NMR (400 MHz, DMSO- d6) δ = 12.81 (s, 1H), 8.32 (s, 1H), 8.09 (d, J = 17.2 Hz, 1H), 7.87 (s, 1H), 7.66 (d, J = 8.4 Hz, 1H), 7.47 (d, J = 8.8 Hz, 1H), 7.13 (d, J = 17.2 Hz, 1H), 4.94-4.87 (m, 1H), 4.84 (t, J = 5.6 Hz, 1H), 4.54-4.29 (m, 2H), 4.08-3.98 (m, 2H), 3.96- 3.83 (m, 2H), 3.72-3.68 (m, 3H), 3.67-3.62 (m, 2H), 3.10- 2.99 (m, 2H), 2.33 (s, 3H), 1.41-1.36 (m, 6H) |
| 226 | 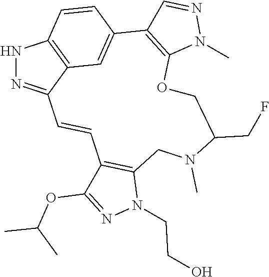 [SMILES: CC(C)Oc1nn(CCO)c2c1/C=C/c1n[nH]c3ccc(cc13)-c1cnn(C)c1OCC(CF)N(C)C2] | 510.1 | 1H NMR (400 MHz, DMSO- d6) δ = 12.83 (s, 1H), 8.44 (s, 1H), 7.92 (s, 1H), 7.76 (d, J = 16.8 Hz, 1H), 7.72-7.64 (m, 1H), 7.47 (d, J = 8.4 Hz, 1H), 7.24 (d, J = 16.8 Hz, 1H), 5.16- 4.75 (m, 5H), 4.40-4.32 (m, 2H), 4.17 (d, J = 14.8 Hz, 1H), 4.06-3.99 (m, 2H), 3.95 (d, J = 14.8 Hz, 1H), 3.72 (s, 3H), 3.68 (t, J = 5.6 Hz, 2H), 2.43 (s, 3H), 1.39 (d, J = 6.0 Hz, 6H) |
| 227 | 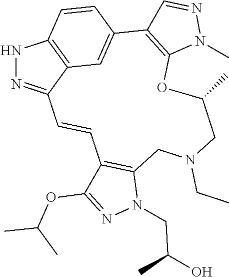 [SMILES: CCN1Cc2c(c(OC(C)C)nn2C[C@H](C)O)/C=C/c2n[nH]c3ccc(cc23)-c2cnn(C)c2O[C@H](C)C1] | 520.3 | 1H NMR (400 MHz, METHANOL-d4) δ = 8.64 (br s, 1H), 7.87 (s, 1H), 7.71 (br d, J = 8.4 Hz, 1H), 7.54 (br d, J = 8.8 Hz, 1H), 7.51-7.44 (m, 2H), 5.46-5.38 (m, 1H), 5.13- 5.03 (m, 1H), 4.71-4.60 (m, 2H), 4.40-4.28 (m, 1H), 4.12- 4.05 (m, 2H), 4.00-3.87 (m, 2H), 3.79 (s, 3H), 3.50 (br d, J = 14.8 Hz, 2H), 1.53 (br d, J = 6.4 Hz, 3H), 1.47 (br t, J = 6.4 Hz, 6H), 1.21 (br d, J = 6.4 Hz, 6H) |
| 228 | 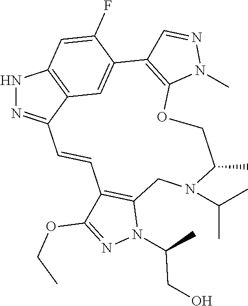 [SMILES: CCOc1nn([C@@H](C)CO)c2c1/C=C/c1n[nH]c3cc(F)c(cc13)-c1cnn(C)c1OC[C@H](C)N(C(C)C)C2] | 538.3 | 1H NMR (400 MHz, METHANOL-d4) δ = 8.77 (d, J = 7.2 Hz, 1H), 7.94-7.84 (m, 1H), 7.70-7.49 (m, 1H), 7.44-7.12 (m, 2H), 4.65-4.54 (m, 6H), 4.41-4.35 (m, 2H), 4.13-4.07 (m, 1H), 3.97-3.94 (m, 2H), 3.94-3.90 (m, 1H), 3.90-3.85 (m, 1H), 3.82 (s, 3H), 3.67-3.65 (m, 1H), 1.49 (t, J = 7.2 Hz, 3H), 1.42 (dd, J = 2.4, 6.8 Hz, 6H), 1.04 (d, J = 5.6 Hz, 3H) |
| 229 | 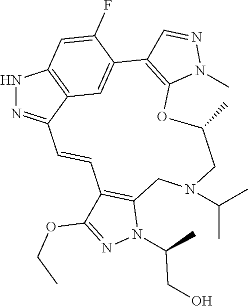 [SMILES: CCOc1nn([C@@H](C)CO)c2c1/C=C/c1n[nH]c3cc(F)c(cc13)-c1cnn(C)c1O[C@H](C)CN(C(C)C)C2] | 538.3 | 1H NMR (400 MHz, METHANOL-d4) δ = 8.66 (br d, J = 7.2 Hz, 1H), 7.84 (d, J = 4.8 Hz, 1H), 7.54-7.44 (m, 1H), 7.42-7.35 (m, 1H), 7.31 (d, J = 11.6 Hz, 1H), 5.46- 5.25 (m, 1H), 4.77-4.73 (m, 1H), 4.60 (br d, J = 14.8 Hz, 2H), 4.48-4.33 (m, 4H), 4.27- 4.15 (m, 1H), 3.84 (br d, J = 3.2 Hz, 1H), 3.79 (s, 3H), 3.29- 3.22 (m, 1H), 1.49 (br t, J = 7.2 Hz, 9H), 1.30 (br d, J = 6.0 Hz, 3H), 1.20 (br d, J = 6.0 Hz, 3H) |
| 230 | 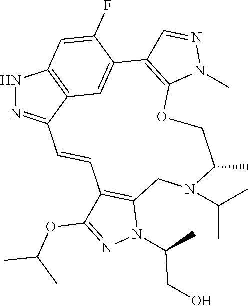 [SMILES: CC(C)Oc1nn([C@@H](C)CO)c2c1/C=C/c1n[nH]c3cc(F)c(cc13)-c1cnn(C)c1OC[C@H](C)N(C(C)C)C2] | 552.2 | 1H NMR (400 MHz, METHANOL-d4) δ = 8.76 (br d, J = 7.6 Hz, 1H), 7.87 (br d, J = 4.4 Hz, 1H), 7.57-7.46 (m, 1H), 7.41-7.31 (m, 1H), 7.26 (br d, J = 12.0 Hz, 1H), 5.11- 5.00 (m, 1H), 4.65-4.44 (m, 2H), 4.16-4.05 (m, 1H), 3.99- 3.92 (m, 1H), 3.83 (s, 3H), 3.73 (br t, J = 6.0 Hz, 3H), 3.70-3.65 (m, 1H), 3.23-3.11 (m, 1H), 1.45 (br dd, J = 6.4, 8.0 Hz, 12H), 1.35-1.25 (m, 6H) |
| 231 | 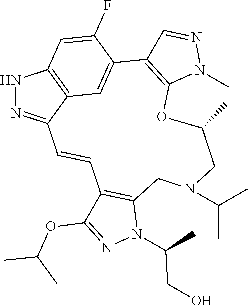 [SMILES: CC(C)Oc1nn([C@@H](C)CO)c2c1/C=C/c1n[nH]c3cc(F)c(cc13)-c1cnn(C)c1O[C@H](C)CN(C(C)C)C2] | 552.3 | 1H NMR (400 MHz, METHANOL-d4) δ = 8.66 (br d, J = 7.2 Hz, 1H), 7.84 (d, J = 4.8 Hz, 1H), 7.66-7.44 (m, 1H), 7.43-7.37 (m, 1H), 7.31 (d, J = 11.6 Hz, 1H), 5.57- 5.32 (m, 1H), 5.13-5.04 (m, 1H), 4.81-4.70 (m, 1H), 4.65- 4.53 (m, 1H), 4.46-4.30 (m, 2H), 4.26-4.12 (m, 1H), 3.85- 3.81 (m, 1H), 3.79 (s, 3H), 3.76-3.70 (m, 1H), 3.28-3.21 (m, 1H), 1.45 (dd, J = 6.0, 9.6 Hz, 12H), 1.30 (br d, J = 5.6 Hz, 3H), 1.19 (br d, J = 6.0 Hz, 3H |
| 232 | 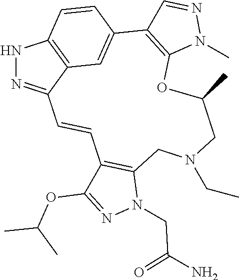 [SMILES: CCN1Cc2c(c(OC(C)C)nn2CC(N)=O)/C=C/c2n[nH]c3ccc(cc23)-c2cnn(C)c2O[C@@H](C)C1] | 519.2 | 1H NMR (400 MHz, METHANOL-d4) δ = 8.65 (br s, 1H), 8.19 (br d, J = 16.8 Hz, 1H), 7.83 (s, 1H), 7.68 (br d, J = 8.8 Hz, 1H), 7.49 (br d, J = 8.4 Hz, 1H), 7.38-7.32 (m, 1H), 5.05-4.97 (m, 1H), 4.78- 4.75 (m, 2H), 4.63-4.56 (m, 1H), 3.93 (s, 2H), 3.74 (s, 3H), 3.08-3.00 (m, 1H), 2.94-2.86 (m, 1H), 2.80-2.68 (m, 2H), 1.45 (br t, J = 5.2 Hz, 6H), 1.13 (br d, J = 6.0 Hz, 3H), 1.01 (br t, J = 6.8 Hz, 3H) |
| 233 | 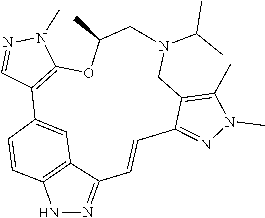 [SMILES: Cc1c2c(nn1C)/C=C/c1n[nH]c3ccc(cc13)-c1cnn(C)c1O[C@@H](C)CN(C(C)C)C2] | 446.1 | 1H NMR (400 MHz, DMSO- d6) δ = 13.22-12.90 (m, 1H), 8.60 (s, 1H), 7.91-7.85 (m, 1H), 7.69 (br d, J = 8.8 Hz, 1H), 7.55 (br d, J = 8.8 Hz, 1H), 7.48 (br d, J = 16.6 Hz, 1H), 7.34-7.25 (m, 1H), 5.58- 5.39 (m, 1H), 4.77 (br d, J = 15.2 Hz, 1H), 4.33-4.21 (m, 1H), 3.94-3.86 (m, 2H), 3.83 (s, 3H), 3.70 (s, 3H), 3.15- 3.03 (m, 1H), 2.35 (s, 3H), 1.50-1.41 (m, 6H), 1.06 (br d, J = 6.0 Hz, 3H) |
| 234 | 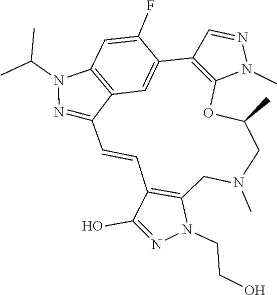 [SMILES: CC(C)n1nc2c3cc(c(F)cc31)-c1cnn(C)c1O[C@@H](C)CN(C)Cc1c(c(O)nn1CCO)/C=C/2] | 510.1 | 1H NMR (400 MHz, METHANOL-d4) δ = 8.65 (br d, J = 7.2 Hz, 1H), 8.10 (d, J = 17.2 Hz, 1H), 7.85 (d, J = 5.2 Hz, 1H), 7.39 (d, J = 12.4 Hz, 1H), 7.33 (d, J = 17.2 Hz, 1H), 4.85-4.81 (m, 1H), 4.81-4.76 (m, 1H), 4.38-4.33 (m, 2H), 4.01-3.95 (m, 3H), 3.76 (s, 3H), 3.74-3.69 (m, 1H), 3.06- 2.99 (m, 1H), 2.86 (dd, J = 7.2, 14.7 Hz, 1H), 2.38 (s, 3H), 1.55 (d, J = 6.8 Hz, 6H), 1.13 (d, J = 6.4 Hz, 3H) |
| 235 | 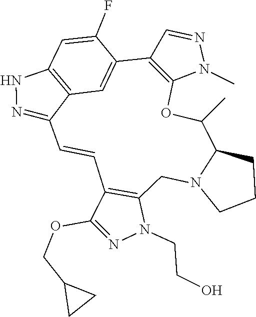 [SMILES: CC1Oc2c(cnn2C)-c2cc3c(n[nH]c3cc2F)/C=C/c2c(OCC3CC3)nn(CCO)c2CN2CCC[C@H]12] | 548.3 | 1H NMR (400 MHz, METHANOL-d4) δ = 8.91 (br d, J = 1.2 Hz, 1H), 8.32 (d, J = 17.2 Hz, 1H), 7.87 (d, J = 4.4 Hz, 1H), 7.32 (br d, J = 17.2 Hz, 1H), 7.22 (d, J = 12.4 Hz, 1H), 4.51-4.42 (m, 1H), 4.33- 4.24 (m, 1H), 4.23-4.03 (m, 5H), 3.96 (d, J = 14.4 Hz, 1H), 3.83 (br s, 2H), 3.81 (s, 3H), 3.08 (td, J = 4.4, 8.8 Hz, 1H), 3.03-2.95 (m, 1H), 2.53-2.43 (m, 1H), 2.26-2.09 (m, 1H), 1.91-1.74 (m, 2H), 1.44-1.36 (m, 1H), 1.33 (br d, J = 4.8 Hz, 3H), 0.71-0.62 (m, 2H), 0.42 (q, J = 4.8 Hz, 2H) |
| 236 | 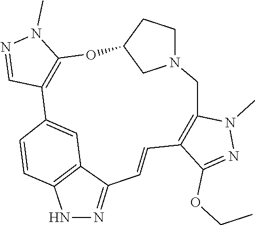 [SMILES: CCOc1nn(C)c2c1/C=C/c1n[nH]c3ccc(cc13)-c1cnn(C)c1O[C@@H]1CCN(C2)C1] | 446.1 | 1H NMR (400 MHz, DMSO- d6) δ = 8.65 (s, 1H), 8.16 (d, J = 17.2 Hz, 1H), 7.88 (s, 1H), 7.67-7.61 (m, 1H), 7.44 (d, J = 8.8 Hz, 1H), 7.22 (d, J = 17.2 Hz, 1H), 5.21 (t, J = 5.6 Hz, 1H), 4.31-4.20 (m, 3H), 3.79 (s, 3H), 3.63 (s, 3H), 3.59- 3.53 (m, 2H), 3.25-3.16 (m, 1H), 2.56 (dd, J = 4.8, 10.8 Hz, 2H), 2.10-1.98 (m, 2H), 1.41 (t, J = 7.2 Hz, 3H) |
| 237 | 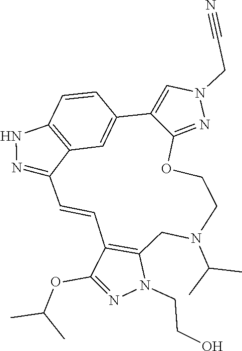 [SMILES: CC(C)Oc1nn(CCO)c2c1/C=C/c1n[nH]c3ccc(cc13)-c1cn(CC#N)nc1OCCN(C(C)C)C2] | 531.1 | 1H NMR (400 MHz, METHANOL-d4) δ = 9.28 (s, 1H), 8.30 (d, J = 17.2 Hz, 1H), 7.91 (s, 1H), 7.54 (d, J = 8.4 Hz, 1H), 7.43 (br d, J = 9.2 Hz, 1H), 7.39 (d, J = 17.2 Hz, 1H), 5.16 (s, 2H), 5.04-4.94 (m, 1H), 4.58-4.52 (m, 2H), 4.14 (t, J = 5.2 Hz, 2H), 3.95 (s, 2H), 3.84 (t, J = 5.2 Hz, 2H), 3.07-3.01 (m, 1H), 3.00-2.96 (m, 2H), 1.45 (d, J = 6.0 Hz, 6H), 0.98 (d, J = 6.4 Hz, 6H) |
| 238 | 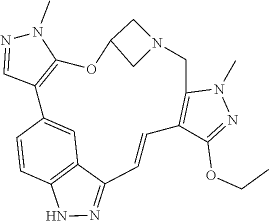 [SMILES: CCOc1nn(C)c2c1/C=C/c1n[nH]c3ccc(cc13)-c1cnn(C)c1OC1CN(C2)C1] | 432.0 | 1H NMR (400 MHz, DMSO- d6) δ = 12.74 (s, 1H), 8.97 (s, 1H), 8.29 (d, J = 17.2 Hz, 1H), 7.82 (s, 1H), 7.56 (dd, J = 1.2, 8.8 Hz, 1H), 7.50-7.44 (m, 1H), 7.00 (d, J = 17.2 Hz, 1H), 5.38 (t, J = 5.2 Hz, 1H), 4.25 (d, J = 7.2 Hz, 2H), 3.87 (s, 2H), 3.74 (s, 3H), 3.65 (s, 3H), 3.52 (s, 2H), 3.46-3.43 (m, 2H), 1.41 (t, J = 7.2 Hz, 3H) |
| 239 | 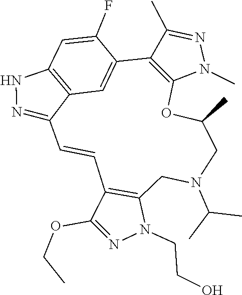 [SMILES: CCOc1nn(CCO)c2c1/C=C/c1n[nH]c3cc(F)c(cc13)-c1c(C)nn(C)c1O[C@@H](C)CN(C(C)C)C2] | 538.3 | 1H NMR (400 MHz, DMSO- d6) δ = 12.87-12.80 (m, 1H), 8.27-8.17 (m, 1H), 7.90-7.78 (d, J = 16.8 Hz, 1H), 7.36- 7.29 (m, 1H), 7.03-6.95 (d, J = 16.8 Hz, 1H), 4.88-4.82 (m, 1H), 4.36-4.30 (m, 1H), 4.26 (q, J = 7.2 Hz, 2H), 4.03-3.99 (m, 3H), 3.68-3.64 (m, 2H), 3.64 (s, 3H), 3.48-3.42 (m, 1H), 2.83-2.71 (m, 1H), 2.63 (br d, J = 6.4 Hz, 1H), 2.14- 2.12 (m, 2H), 1.43-1.38 (m, 3H), 1.20-1.17 (m, 2H), 0.88 (br d, J = 6.0 Hz, 3H), 0.80- 0.71 (m, 6H) |
| 240 | 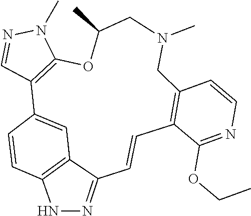 [SMILES: CCOc1nccc2c1/C=C/c1n[nH]c3ccc(cc13)-c1cnn(C)c1O[C@@H](C)CN(C)C2] | 445.0 | 1H NMR (400 MHz, DMSO- d6) δ = 13.09 (s, 1H), 8.44 (s, 1H), 8.39 (s, 1H), 7.99 (d, J = 4.8 Hz, 1H), 7.93 (d, J = 17.2 Hz, 1H), 7.80 (s, 1H), 7.65 (d, J = 8.8 Hz, 1H), 7.53 (d, J = 8.4 Hz, 1H), 6.93 (d, J = 4.8 Hz, 1H), 4.88 (t, J = 6.4 Hz, 1H), 4.52 (s, 2H), 4.01 (d, J = 12.0 Hz, 1H), 3.66 (s, 3H), 3.58 (d, J = 12.0 Hz, 1H), 2.87-2.80 (m, 1H), 2.74-2.67 (m, 1H), 2.20 (s, 3H), 1.44 (t, J = 7.2 Hz, 3H), 1.42-1.42 (m, 1H), 0.95 (d, J = 6.0 Hz, 3H) |
| 241 | 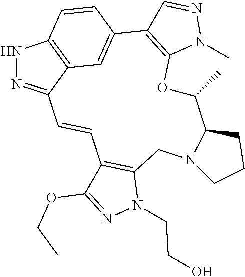 [SMILES: CCOc1nn(CCO)c2c1/C=C/c1n[nH]c3ccc(cc13)-c1cnn(C)c1O[C@H](C)[C@H]1CCCN1C2] | 504.2 | 1H NMR (400 MHz, METHANOL-d4) δ = 8.85- 8.35 (m, 1H), 7.96 (br d, J = 16.6 Hz, 1H), 7.81 (s, 1H), 7.64 (br d, J = 8.6 Hz, 1H), 7.47 (d, J = 8.8 Hz, 1H), 7.27 (d, J = 16.9 Hz, 1H), 4.44- 4.21 (m, 5H), 4.18-4.05 (m, 2H), 3.97-3.82 (m, 4H), 3.81- 3.76 (m, 3H), 2.99-2.81 (m, 1H), 2.59-2.50 (m, 1H), 2.22- 2.11 (m, 1H), 1.91-1.73 (m, 2H), 1.49 (t, J = 7.2 Hz, 3H), 1.05 (br d, J = 6.4 Hz, 3H) |
| 241 | 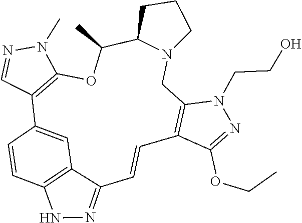 | 504.3 | 1H NMR (400 MHz, METHANOL-d4) δ = 8.88- 8.70 (m, 1H), 7.89-7.80 (m, 1H), 7.77-7.61 (m, 2H), 7.57- 7.48 (m, 1H), 7.43-7.31 (m, 1H), 5.11-4.91 (m, 1H), 4.79 (br s, 2H), 4.44-4.36 (m, 2H), 4.30-4.20 (m, 2H), 4.02-3.86 (m, 3H), 3.84 (s, 3H), 3.60- 3.47 (m, 1H), 3.43-3.31 (m, 2H), 2.49-2.31 (m, 1H), 2.07 (br s, 2H), 1.50 (dt, J = 1.8, 7.2 Hz, 3H), 1.41-1.26 (m, 3H) |
| 242 | 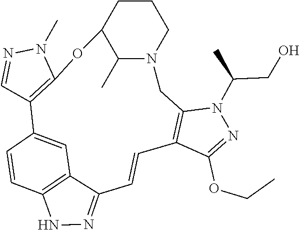 | 504.2 | 1H NMR (400 MHz, METHANOL-d4) δ = 8.24- 8.21 (m, 1H), 8.05-8.02 (m, 1H), 7.79 (dd, J = 1.4, 8.8 Hz, 1H), 7.59-7.52 (m, 2H), 7.37- 7.31 (m, 1H), 5.50-5.40 (m, 1H), 5.20 (br d, J = 15.6 Hz, 1H), 4.47-4.37 (m, 4H), 4.18- 4.09 (m, 2H), 4.03-3.93 (m, 3H), 3.79-3.76 (m, 3H), 2.30- 2.16 (m, 2H), 2.11-1.95 (m, 4H), 1.65 (d, J = 6.8 Hz, 3H), 1.55-1.50 (m, 3H) |
| 243 | 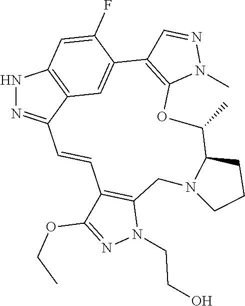 [SMILES: CCOc1nn(CCO)c2c1/C=C/c1n[nH]c3cc(F)c(cc13)-c1cnn(C)c1O[C@H](C)[C@H]1CCCN1C2] | 522.1 | 1H NMR (400 MHz, METHANOL-d4) δ = 9.03- 8.62 (m, 1H), 7.92 (br d, J = 17.6 Hz, 1H), 7.81 (d, J = 4.8 Hz, 1H), 7.28-7.18 (m, 2H), 4.58 (br s, 2H), 4.38-4.32 (m, 3H), 4.11 (td, J = 4.4, 14.4 Hz, 2H), 3.88 (br s, 2H), 3.86 (br d, J = 5.2 Hz, 2H), 3.81 (s, 3H), 2.88-2.79 (m, 1H), 2.56-2.48 (m, 1H), 2.22-2.15 (m, 1H), 1.79-1.74 (m, 1H), 1.49 (t, J = 7.2 Hz, 3H), 1.35-1.27 (m, 3H), 1.03 (br d, J = 6.4 Hz, 3H |
| 244 | 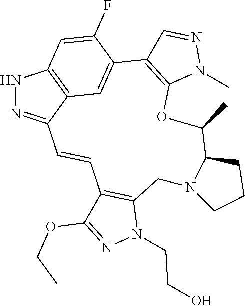 [SMILES: CCOc1nn(CCO)c2c1/C=C/c1n[nH]c3cc(F)c(cc13)-c1cnn(C)c1O[C@@H](C)[C@H]1CCCN1C2] | 522.0 | 1H NMR (400 MHz, METHANOL-d4) δ = 9.02- 8.88 (m, 1H), 8.35 (br d, J = 17.2 Hz, 1H), 7.94-7.84 (m, 1H), 7.31 (br d, J = 16.4 Hz, 1H), 7.25 (d, J = 12.4 Hz, 1H), 4.54-4.46 (m, 1H), 4.40-4.33 (m, 2H), 4.24-4.17 (m, 1H), 4.15-4.08 (m, 1H), 3.99 (br d, J = 14.4 Hz, 1H), 3.88-3.85 (m, 2H), 3.84 (s, 3H), 3.14- 3.08 (m, 1H), 3.06-2.98 (m, 1H), 2.56-2.44 (m, 1H), 2.30- 2.12 (m, 2H), 1.94-1.79 (m, 2H), 1.52 (t, J = 7.2 Hz, 3H), 1.41-1.28 (m, 6H) |
| 245 | 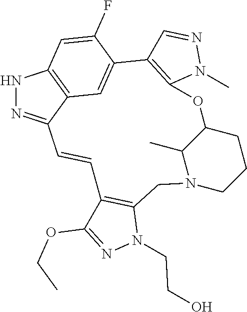 [SMILES: CCOc1nn(CCO)c2c1/C=C/c1n[nH]c3cc(F)c(cc13)-c1cnn(C)c1OC1CCCN(C2)C1C] | 522.2 | 1H NMR (400 MHz, METHANOL-d4) δ = 8.99- 8.91 (m, 1H), 8.45-8.22 (m, 1H), 7.92-7.84 (m, 1H), 7.41- 7.26 (m, 1H), 7.24 (d, J = 12.4 Hz, 1H), 4.49 (br s, 1H), 4.36-4.34 (m, 2H), 4.18 (br d, J = 2.0 Hz, 1H), 4.12 (br t, J = 4.8 Hz, 1H), 4.00 (s, 1H), 3.85 (br s, 2H), 3.83 (s, 3H), 3.11- 3.08 (m, 1H), 3.02-2.99 (m, 1H), 2.52-2.48 (m, 1H), 1.89- 1.82 (m, 2H), 1.61 (ddd, J = 3.2, 6.4, 11.2 Hz, 2H), 1.50 (t, J = 7.2 Hz, 3H), 1.34 (br s, 3H), 1.29 (br s, 3H) |
| 246 | 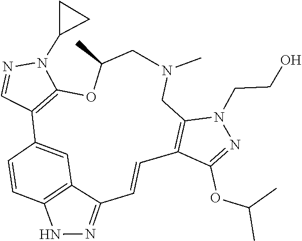 | 518.2 | 1H NMR (400 MHz, DMSO- d6) δ = 12.78 (s, 1H), 8.53 (s, 1H), 8.13 (d, J = 17.2 Hz, 1H), 7.82 (s, 1H), 7.65 (d, J = 8.8 Hz, 1H), 7.45 (d, J = 8.8 Hz, 1H), 7.16 (d, J = 17.2 Hz, 1H), 4.91 (quin, J = 6.0 Hz, 1H), 4.87-4.78 (m, 2H), 4.09-3.97 (m, 2H), 3.96-3.90 (m, 1H), 3.87-3.80 (m, 1H), 3.70-3.60 (m, 3H), 3.00 (d, J = 13.6 Hz, 1H), 2.82 (dd, J = 6.8, 14.4 Hz, 1H), 2.35 (s, 3H), 1.39 (t, J = 6.4 Hz, 6H), 1.15-1.07 (m, 4H), 1.07-1.03 (m, 1H), 0.99 (d, J = 7.2 Hz, 2H) |
| 247 | 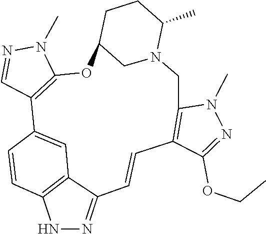 | 474.2 | 1H NMR (400 MHz, DMSO- d6) δ 12.90-12.64 (m, 1H), 8.49-8.39 (m, 1H), 7.66-7.51 (m, 1H), 7.39-7.25 (m, 2H), 7.16-7.05 (m, 1H), 7.01-6.85 (m, 1H), 4.38-4.27 (m, 2H), 4.21-4.04 (m, 1H), 3.77-3.55 (m, 2H), 3.40 (br d, J = 2.8 Hz, 6H), 3.20-3.06 (m, 2H), 2.79- 2.72 (m, 2H), 1.46-1.41 (m, 3H), 1.19-1.10 (m, 2H), 1.06- 0.97 (m, 3H), 0.55-0.36 (m, 1H) |
| 248 | 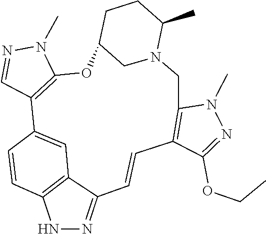 | 474.3 | 1H NMR (400 MHz, DMSO- d6) δ 13.01-12.58 (m, 1H), 8.48-8.38 (m, 1H), 7.66-7.49 (m, 1H), 7.41-7.23 (m, 2H), 7.20-7.04 (m, 1H), 7.03-6.78 (m, 1H), 4.32 (td, J = 7.2, 14.4 Hz, 2H), 4.22-4.00 (m, 1H), 3.64 (br s, 2H), 3.55- 3.42 (m, 6H), 3.16-3.00 (m, 2H), 2.75 (br s, 2H), 1.42 (br t, J = 6.4 Hz, 3H), 1.25-1.11 (m, 2H), 1.07-0.92 (m, 3H), 0.57-0.35 (m, 1H) |
| 249 | 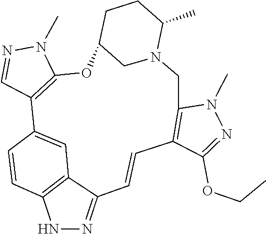 | 474.1 | 1H NMR (400 MHz, DMSO- d6) δ 12.81 (s, 1H), 7.96 (s, 1H), 7.74-7.68 (m, 1H), 7.46 (d, J = 8.8 Hz, 1H), 7.16 (d, J = 16.8 Hz, 1H), 4.98-4.85 (m, 1H), 4.31-4.23 (m, 2H), 4.13 (br d, J = 15.6 Hz, 1H), 3.74 (s, 2H), 3.64 (s, 3H), 3.31 (s, 3H), 3.15-3.05 (m, 1H), 2.95-2.79 (m, 2H), 1.93-1.80 (m, 1H), 1.75-1.61 (m, 2H), 1.49 (dt, J = 3.6, 6.0 Hz, 1H), 1.41 (t, J = 7.2 Hz, 3H), 1.14 (br d, J = 6.4 Hz, 3H) |
| 250 | 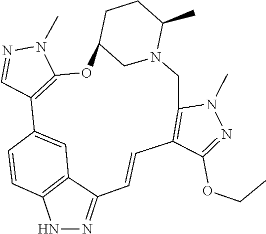 | 474.1 | 1H NMR (400 MHz, DMSO- d6) δ 12.96-12.68 (m, 1H), 7.96 (s, 1H), 7.46 (d, J = 8.8 Hz, 1H), 7.16 (d, J = 16.8 Hz, 1H), 4.99-4.86 (m, 1H), 4.32- 4.23 (m, 2H), 4.13 (br d, J = 15.6 Hz, 1H), 3.74 (s, 2H), 3.64 (s, 3H), 3.31 (br s, 3H), 3.16-3.03 (m, 1H), 2.87 (br d, J = 9.6 Hz, 2H), 1.93-1.81 (m, 1H), 1.76-1.62 (m, 2H), 1.54-1.45 (m, 1H), 1.41 (t, J = 7.2 Hz, 3H), 1.14 (br d, J = 6.4 Hz, 3H) |
| 251 | 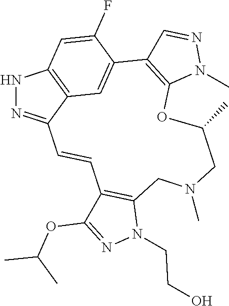 [SMILES: CC(C)Oc1nn(CCO)c2c1/C=C/c1n[nH]c3cc(F)c(cc13)-c1cnn(C)c1O[C@H](C)CN(C)C2] | 510.1 | 1H NMR (400 MHz, DMSO- d6) δ = 13.44-12.41 (m, 1H), 8.57-8.51 (m, 1H), 8.05 (d, J = 17.2 Hz, 1H), 7.75 (d, J = 5.2 Hz, 1H), 7.35 (br d, J = 12.0 Hz, 1H), 7.12 (d, J = 17.2 Hz, 1H), 4.90 (s, 2H), 4.73-4.63 (m, 1H), 4.09-3.98 (m, 2H), 3.94-3.81 (m, 2H), 3.73-3.68 (m, 3H), 3.66-3.60 (m, 2H), 3.02-2.92 (m, 1H), 2.85-2.74 (m, 1H), 2.30 (s, 3H), 1.38 (t, J = 6.4 Hz, 6H), 1.04 (br d, J = 6.0 Hz, 3H) |
| 252 | 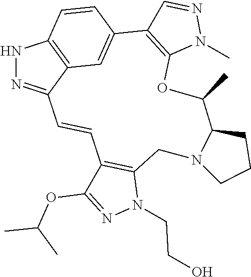 [SMILES: CC(C)Oc1nn(CCO)c2c1/C=C/c1n[nH]c3ccc(cc13)-c1cnn(C)c1O[C@@H](C)[C@H]1CCCN1C2] | 518.2 | 1H NMR (400 MHz, METHANOL-d4) δ = 8.51 (br s, 1H), 7.83 (s, 1H), 7.72 (d, J = 8.9 Hz, 1H), 7.57 (d, J = 8.8 Hz, 1H), 7.47-7.40 (m, 1H), 7.40-7.31 (m, 1H), 5.62 (br d, J = 2.4 Hz, 1H), 5.17-5.05 (m, 2H), 4.72-4.64 (m, 1H), 4.33-4.21 (m, 2H), 4.01 (td, J = 4.0, 11.4 Hz, 2H), 3.91 (td, J = 5.5, 11.3 Hz, 1H), 3.82 (s, 3H), 3.80-3.74 (m, 1H), 3.66- 3.51 (m, 1H), 2.71-2.57 (m, 1H), 2.57-2.45 (m, 1H), 2.43- 2.32 (m, 1H), 2.21 (br dd, J = 4.3, 5.9 Hz, 1H), 1.47 (dd, J = 6.1, 17.6 Hz, 6H), 1.23 (br d, |
| | | | J = 6.4 Hz, 3H) |
| 253 | 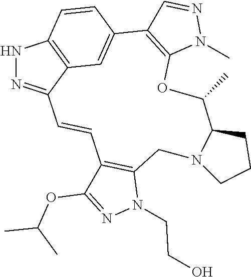 [SMILES: CC(C)Oc1nn(CCO)c2c1/C=C/c1n[nH]c3ccc(cc13)-c1cnn(C)c1O[C@H](C)[C@H]1CCCN1C2] | 518.2 | 1H NMR (499 MHz, METHANOL-d4) δ ppm 8.84- 8.99 (m, 1 H), 8.20-8.40 (m, 1 H), 7.87 (s, 1 H), 7.69 (d, J = 8.76 Hz, 1 H), 7.47 (d, J = 8.76 Hz, 1 H), 7.38 (br d, J = 16.97 Hz, 1 H), 4.99 (dt, J = 12.11, 6.13 Hz, 1 H), 4.48- 4.58 (m, 1 H), 4.07-4.31 (m, 3 H), 3.92-4.06 (m, 1 H), 3.82- 3.89 (m, 2 H), 3.81 (s, 3 H), 2.97-3.20 (m, 2 H), 2.44- 2.62 (m, 1 H), 2.07-2.31 (m, 2 H), 1.95 (br d, J = 5.48 Hz, 1 H), 1.85 (br s, 1 H), 1.46 (dd, J = 6.30, 1.37 Hz, 6 H), 1.35- 1.43 (m, 3 H) |
| 254 | 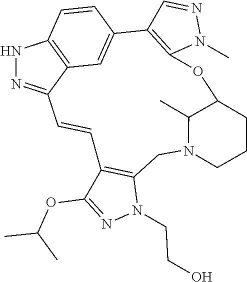 [SMILES: CC(C)Oc1nn(CCO)c2c1/C=C/c1n[nH]c3ccc(cc13)-c1cnn(C)c1OC1CCCN(C2)C1C] | 518.1 | 1H NMR (400 MHz, METHANOL-d4) δ = 8.21 (s, 1H), 8.03 (s, 1H), 7.79 (d, J = 8.8 Hz, 1H), 7.61-7.55 (m, 1H), 7.53 (d, J = 8.9 Hz, 1H), 7.32 (d, J = 16.3 Hz, 1H), 5.48- 5.40 (m, 1H), 5.20 (br d, J = 15.3 Hz, 1H), 5.08 (td, J = 6.1, 12.2 Hz, 1H), 4.43-4.33 (m, 2H), 4.32-4.25 (m, 1H), 4.18- 4.09 (m, 1H), 3.97 (br d, J = 5.9 Hz, 2H), 3.77 (s, 3H), 3.70- 3.62 (m, 2H), 2.25 (br dd, J = 4.6, 11.6 Hz, 1H), 2.10-1.97 (m, 3H), 1.66 (d, J = 6.9 Hz, 3H), 1.49 (dd, J = 3.1, 6.1 Hz, 6H) 518.1 |
| 255 | 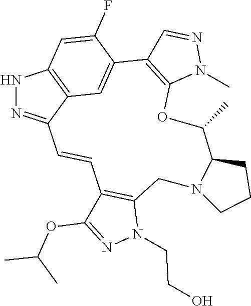 [SMILES: CC(C)Oc1nn(CCO)c2c1/C=C/c1n[nH]c3cc(F)c(cc13)-c1cnn(C)c1O[C@H](C)[C@H]1CCCN1C2] | 536.4 | 1H NMR (400 MHz, METHANOL-d4) δ = 8.92 (br d, J = 2.8 Hz, 1H), 8.30 (br d, J = 16.8 Hz, 1H), 7.93-7.80 (m, 1H), 7.37-7.26 (m, 1H), 7.22 (d, J = 12.1 Hz, 1H), 5.03- 4.93 (m, 1H), 4.56-4.41 (m, 1H), 4.35-4.24 (m, 1H), 4.24- 4.15 (m, 1H), 4.14-4.05 (m, 1H), 3.96 (br d, J = 14.0 Hz, 1H), 3.86-3.83 (m, 2H), 3.81 (s, 3H), 3.08 (td, J = 4.4, 8.8 Hz, 1H), 3.04-2.95 (m, 1H), 2.57-2.40 (m, 1H), 2.26-2.12 (m, 1H), 2.11-1.94 (m, 1H), 1.92-1.72 (m, 2H), 1.45 (d, J = 6.0 Hz, 6H), 1.33 (br d, J = 4.0 Hz, 3H) |
| 256 | 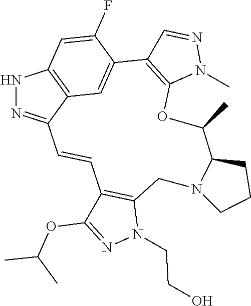 [SMILES: CC(C)Oc1nn(CCO)c2c1/C=C/c1n[nH]c3cc(F)c(cc13)-c1cnn(C)c1O[C@@H](C)[C@H]1CCCN1C2] | 536.2 | 1H NMR (400 MHz, METHANOL-d4) δ = 8.70- 8.32 (m, 1H), 7.81 (d, J = 5.0 Hz, 1H), 7.43-7.23 (m, 3H), 5.69-5.32 (m, 1H), 5.06 (td, J = 6.0, 12.0 Hz, 2H), 4.73- 4.47 (m, 1H), 4.24 (br s, 2H), 3.97 (br d, J = 11.2 Hz, 2H), 3.88 (td, J = 5.6, 11.2 Hz, 1H), 3.82 (s, 3H), 3.76-3.64 (m, 1H), 3.62-3.38 (m, 1H), 2.70- 2.50 (m, 1H), 2.50-2.24 (m, 2H), 2.24-2.00 (m, 1H), 1.45 (dd, J = 6.0, 14.8 Hz, 6H), 1.18 (br d, J = 2.0 Hz, 3H) |
| 257 | 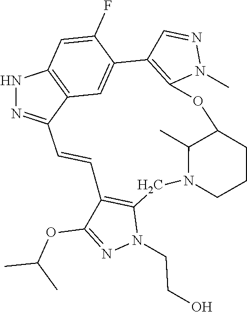 [SMILES: CC(C)Oc1nn(CCO)c2c1/C=C/c1n[nH]c3cc(F)c(cc13)-c1cnn(C)c1OC1CCCN(C2)C1C] | 536.2 | 1H NMR (400 MHz, METHANOL-d4) δ = 8.32 (d, J = 7.2 Hz, 1H), 8.05 (d, J = 4.7 Hz, 1H), 7.55 (d, J = 16.4 Hz, 1H), 7.33-7.24 (m, 2H), 5.49-5.40 (m, 1H), 5.16 (br d, J = 15.4 Hz, 1H), 5.08 (td, J = 6.0, 12.4 Hz, 1H), 4.44-4.33 (m, 2H), 4.33-4.23 (m, 1H), 4.10 (quin, J = 6.4 Hz, 1H), 3.97 (br d, J = 5.6 Hz, 2H), 3.79 (s, 3H), 3.71-3.55 (m, 2H), 2.31-2.17 (m, 1H), 2.03 (br s, 2H), 2.00-1.92 (m, 1H), 1.66 (d, J = 6.8 Hz, 3H), 1.48 (dd, J = 2.4, 6.0 Hz, 6H) |
| 258 | 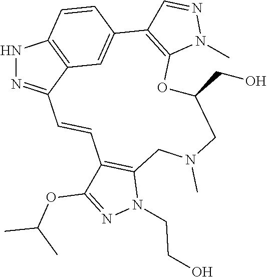 [SMILES: CC(C)Oc1nn(CCO)c2c1/C=C/c1n[nH]c3ccc(cc13)-c1cnn(C)c1O[C@@H](CO)CN(C)C2] | 508.1 | 1H NMR (400 MHz, METHANOL-d4) δ = 8.47 (s, 1H), 8.28 (d, J = 16.0 Hz, 1H), 7.80 (s, 1H), 7.66 (dd, J = 4.4, 8.0 Hz, 1H), 7.48 (d, J = 8.0 Hz, 1H), 7.31 (d, J = 16.0 Hz, 1H), 4.97 (td, J = 6.0, 12.0 Hz, 1H), 4.72 (br s, 1H), 4.16- 4.07 (m, 2H), 3.99-3.88 (m, 2H), 3.83 (t, J = 6.0 Hz, 2H), 3.78 (s, 3H), 3.66-3.58 (m, 1H), 3.47 (dd, J = 2.0, 12.0 Hz, 1H), 3.11-3.05 (m, 2H), 2.44 (s, 3H), 1.45 (t, J = 6.0 Hz, 6H) |
| 259 | 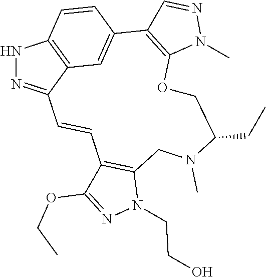 [SMILES: CCOc1nn(CCO)c2c1/C=C/c1n[nH]c3ccc(cc13)-c1cnn(C)c1OC[C@H](CC)N(C)C2] | 492.1 | 1H NMR (400 MHz, DMSO- d6) δ = 12.83 (s, 1H), 8.49 (s, 1H), 7.92 (s, 1H), 7.73-7.62 (m, 2H), 7.47 (d, J = 9.4 Hz, 1H), 7.20 (d, J = 17.4 Hz, 1H), 4.92-4.85 (m, 1H), 4.47-4.38 (m, 1H), 4.33-4.24 (m, 2H), 4.17-4.06 (m, 3H), 4.03-3.93 (m, 1H), 3.89-3.80 (m, 1H), 3.74 (s, 3H), 3.72-3.67 (m, 2H), 2.90-2.83 (m, 1H), 2.33 (s, 3H), 1.76-1.67 (m, 2H), 1.42 (t, J = 7.0 Hz, 3H), 0.95- 0.88 (m, 3H) |
| 260 | 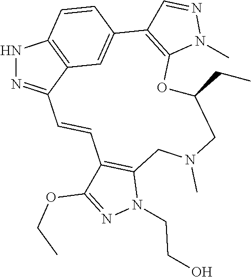 [SMILES: CCOc1nn(CCO)c2c1/C=C/c1n[nH]c3ccc(cc13)-c1cnn(C)c1O[C@@H](CC)CN(C)C2] | 492.1 | 1H NMR (400 MHz, DMSO- d6) δ = 12.80 (s, 1H), 8.52- 8.42 (m, 1H), 8.17 (d, J = 17.2 Hz, 1H), 7.86 (s, 1H), 7.71-7.62 (m, 1H), 7.46 (d, J = 8.4H z, 1H), 7.16 (d, J = 17.4 Hz, 1H), 4.86 (t, J = 5.4 Hz, 1H), 4.54-4.40 (m, 1H), 4.32-4.25 (m, 2H), 4.08- 4.00 (m, 2H), 3.99-3.93 (m, 1H), 3.90-3.80 (m, 1H), 3.68 (s, 3H), 3.65 (br d, J = 5.6 Hz, 2H), 3.01 (br d, J = 14.8 Hz, 1H), 2.88-2.81 (m, 1H), 2.35 (br s, 3H), 1.76-1.57 (m, 1H), 1.42 (t, J = 7.0 Hz, 4H), 0.72 (br t, J = 7.4 Hz, 3H) |
| 261 | 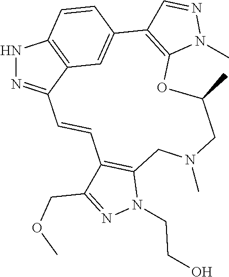 [SMILES: COCc1nn(CCO)c2c1/C=C/c1n[nH]c3ccc(cc13)-c1cnn(C)c1O[C@@H](C)CN(C)C2] | 478.1 | 1H NMR (400 MHz, METHANOL-d4) δ = 8.53 (s, 1H), 8.41 (d, J = 17.2 Hz, 1H), 7.80 (s, 1H), 7.66 (d, J = 8.8 Hz, 1H), 7.49 (d, J = 8.8 Hz, 1H), 7.14 (d, J = 17.2 Hz, 1H), 4.95-4.90 (m, 1H), 4.65-4.53 (m, 2H), 4.33-4.16 (m, 2H), 4.04-3.92 (m, 2H), 3.91-3.82 (m, 2H), 3.72 (s, 3H), 3.48 (s, 3H), 2.95-2.79 (m, 2H), 2.37 (s, 3H), 1.07 (d, J = 6.2 Hz, 3H) |
| 262 | 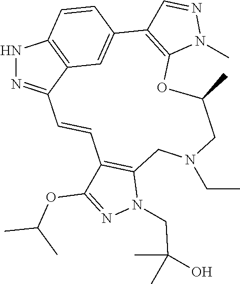 [SMILES: CCN1Cc2c(c(OC(C)C)nn2CC(C)(C)O)/C=C/c2n[nH]c3ccc(cc23)-c2cnn(C)c2O[C@@H](C)C1] | 534.2 | 1H NMR (400 MHz, METHANOL-d4) δ = 8.67 (s, 1H), 8.22 (d, J = 17.2 Hz, 1H), 7.85 (s, 1H), 7.69 (d, J = 8.8 Hz, 1H), 7.50 (d, J = 8.8 Hz, 1H), 7.40-7.29 (m, 1H), 5.02- 4.98 (m, 1H), 4.62 (br s, 1H), 4.16-3.94 (m, 4H), 3.76 (s, 3H), 3.15-2.97 (m, 1H), 2.93 (br d, J = 14.8 Hz, 1H), 2.82- 2.58 (m, 2H), 1.53-1.43 (m, 6H), 1.23 (d, J = 3.6 Hz, 6H), 1.14 (br d, J = 6.4 Hz, 3H), 1.02 (t, J = 7.2 Hz, 3H) |
| 263 | 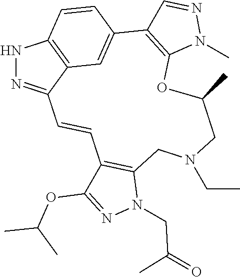 [SMILES: CCN1Cc2c(c(OC(C)C)nn2CC(C)=O)/C=C/c2n[nH]c3ccc(cc23)-c2cnn(C)c2O[C@@H](C)C1] | 518.5 | 1H NMR (400 MHz, METHANOL-d4) δ = 8.65 (s, 1H), 8.20 (d, J = 16.8 Hz, 1H), 7.83 (s, 1H), 7.68 (d, J = 8.8 Hz, 1H), 7.49 (d, J = 8.8 Hz, 1H), 7.35 (d, J = 17.2 Hz, 1H), 4.97 (s, 2H), 4.96-4.92 (m, 1H), 4.60 (s, 2H), 3.92-3.77 (m, 2H), 3.74 (s, 3H), 3.08- 2.98 (m, 1H), 2.88 (br d, J = 15.6 Hz, 1H), 2.77-2.63 (m, 2H), 2.23-2.15 (m, 3H), 1.47- 1.42 (m, 6H), 1.12 (br d, J = 6.4 Hz, 3H), 0.99 (t, J = 7.2 Hz, 3H) |
| 264 | 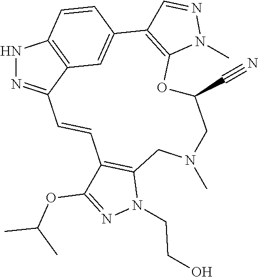 [SMILES: CC(C)Oc1nn(CCO)c2c1/C=C/c1n[nH]c3ccc(cc13)-c1cnn(C)c1O[C@@H](C#N)CN(C)C2] | 503.1 | 1H NMR (400 MHz, METHANOL-d4) δ = 8.54 (s, 1H), 8.18 (d, J = 16.0 Hz, 1H), 7.92 (s, 1H), 7.75 (dd, J = 1.2, 8.8 Hz, 1H), 7.52 (d, J = 8.8 Hz, 1H), 7.35 (d, J = 16.0 Hz, 1H), 5.48 (dd, J = 4.0, 8.0 Hz, 1H), 4.99 (td, J = 6.0, 12.0 Hz, 1H), 4.16-4.08 (m, 2H), 4.04 (d, J = 4.0 Hz, 2H), 3.85 (s, 3H), 3.84-3.80 (m, 2H), 3.38- 3.32 (m, 2H), 2.47 (s, 3H), 1.45 (dd, J = 4.0, 6.0 Hz, 6H) |
| 265 | 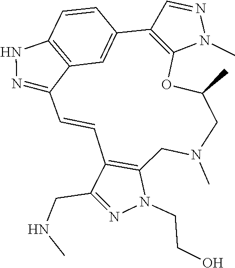 [SMILES: CNCc1nn(CCO)c2c1/C=C/c1n[nH]c3ccc(cc13)-c1cnn(C)c1O[C@@H](C)CN(C)C2] | 477.2 | 1H NMR (400 MHz, METHANOL-d4) δ = 8.53- 8.45 (m, 2H), 7.86 (s, 1H), 7.74-7.69 (m, 1H), 7.56-7.51 (m, 1H), 6.89-6.82 (m, 1H), 4.61-4.59 (m, 1H), 4.53-4.49 (m, 2H), 4.32 (td, J = 2.9, 6.3 Hz, 2H), 4.07 (s, 2H), 3.91 (br s, 2H), 3.77-3.73 (m, 3H), 2.96-2.92 (m, 2H), 2.89 (s, 3H), 2.44-2.39 (m, 3H), 1.11 (d, J = 6.1 Hz, 3H) |
| 266 | 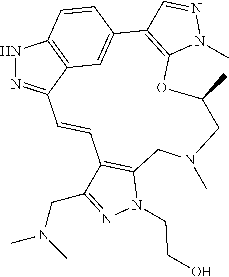 | 491.2 | 1H NMR (400 MHz, METHANOL-d4) δ = 8.60- 8.54 (m, 1H), 8.48-8.41 (m, 1H), 7.86-7.81 (m, 1H), 7.71 (d, J = 9.0 Hz, 1H), 7.57-7.49 (m, 1H), 7.02 (d, J = 17.6 Hz, 1H), 4.61 (br s, 1H), 4.34- 4.27 (m, 4H), 4.01 (br d, J = 11.6 Hz, 2H), 3.93-3.88 (m, 2H), 3.75 (s, 3H), 2.96-2.90 (m, 2H), 2.81 (s, 6H), 2.41 (s, 3H), 1.10 (d, J = 6.1 Hz, 3H) |
| 267 | 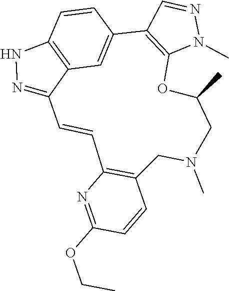 [SMILES: CCOc1ccc2c(n1)/C=C/c1n[nH]c3ccc(cc13)-c1cnn(C)c1O[C@@H](C)CN(C)C2] | 445.0 | 1H NMR (400 MHz, DMSO- d6) δ = 13.19 (s, 1H), 8.54 (s, 1H), 8.50 (d, J = 16.0 Hz, 1H), 7.91-7.86 (m, 2H), 7.71 (dd, J = 1.2, 8.8 Hz, 1H), 7.54 (dd, J = 2.0, 8.4 Hz, 2H), 6.63 (d, J = 8.4 Hz, 1H), 4.90 (t, J = 6.4 Hz, 1H), 4.44 (dq, J = 3.2, 7.2 Hz, 2H), 4.06 (br d, J = 12.5 Hz, 1H), 3.67 (s, 3H), 3.40 (br d, J = 12.4 Hz, 1H), 2.93-2.85 (m, 1H), 2.84-2.75 (m, 1H), 2.22 (s, 3H), 1.40 (d, J = 2.8 Hz, 1H), 1.39-1.36 (m, 2H), 1.01 (d, J = 6.0 Hz, 3H) |
| 268 | 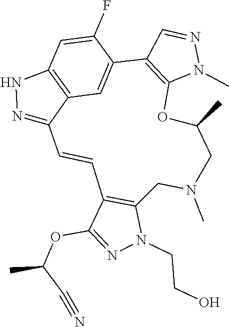 [SMILES: C[C@H](C#N)Oc1nn(CCO)c2c1/C=C/c1n[nH]c3cc(F)c(cc13)-c1cnn(C)c1O[C@@H](C)CN(C)C2] | 521.1 | 1H NMR (400 MHz, METHANOL-d4) δ = 8.61 (br d, J = 7.2 Hz, 1H), 8.24 (br d, J = 17.2 Hz, 1H), 7.84 (d, J = 5.2 Hz, 1H), 7.31-7.13 (m, 2H), 5.59-5.47 (m, 1H), 4.60 (br d, J = 2.8 Hz, 1H), 4.22-4.07 (m, 2H), 4.03-3.92 (m, 2H), 3.90-3.81 (m, 2H), 3.76 (s, 3H), 3.06-2.95 (m, 1H), 2.94- 2.80 (m, 1H), 2.42 (s, 3H), 1.84 (d, J = 6.8 Hz, 3H), 1.12 (br d, J = 6.4 Hz, 3H) |
| 269 | 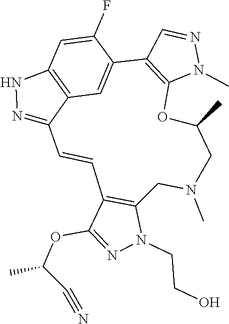 [SMILES: C[C@@H](C#N)Oc1nn(CCO)c2c1/C=C/c1n[nH]c3cc(F)c(cc13)-c1cnn(C)c1O[C@@H](C)CN(C)C2] | 521.1 | 1H NMR (400 MHz, METHANOL-d4) δ = 8.64 (br d, J = 6.8 Hz, 1H), 8.26 (d, J = 17.2 Hz, 1H), 7.84 (d, J = 5.2 Hz, 1H), 7.32-7.10 (m, 2H), 5.59-5.49 (m, 1H), 4.59 (s, 1H), 4.23-4.10 (m, 2H), 4.01- 3.92 (m, 2H), 3.91-3.83 (m, 2H), 3.76 (s, 3H), 3.03-2.98 (m, 1H), 2.92-2.86 (m, 1H), 2.42 (s, 3H), 1.91-1.82 (m, 3H), 1.12 (d, J = 6.4 Hz, 3H) |
| 270 | 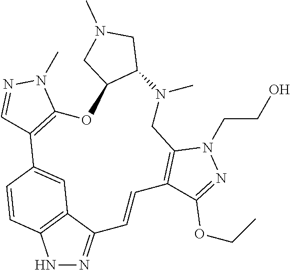 [SMILES: CCOc1nn(CCO)c2c1/C=C/c1n[nH]c3ccc(cc13)-c1cnn(C)c1O[C@H]1CN(C)C[C@@H]1N(C)C2] | 519.0 | 1H NMR (400 MHz, METHANOL-d4) δ = 8.19- 8.15 (m, 1H), 8.07 (br d, J = 16.8 Hz, 1H), 7.75 (s, 1H), 7.57 (d, J = 8.8 Hz, 1H), 7.39 (d, J = 8.8 Hz, 1H), 7.33 (d, J = 17.2 Hz, 1H), 4.86-4.82 (m, 1H), 4.34-4.26 (m, 2H), 4.09- 4.03 (m, 1H), 3.99 (br t, J = 5.2 Hz, 2H), 3.85-3.80 (m, 3H), 3.75 (s, 4H), 3.02 (br t, J = 8.4 Hz, 1H), 2.69 (br d, J = 12.0 Hz, 1H), 2.34 (br t, J = 9.2 Hz, 1H), 2.27 (s, 3H), 2.06 (s, 3H), 2.04-1.99 (m, 1H), 1.49 (t, J = 7.2 Hz, 3H). |
| 271 | 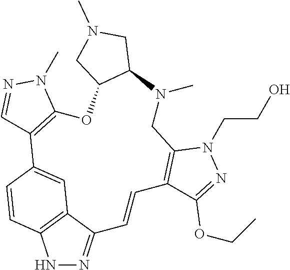 [SMILES: CCOc1nn(CCO)c2c1/C=C/c1n[nH]c3ccc(cc13)-c1cnn(C)c1O[C@@H]1CN(C)C[C@H]1N(C)C2] | 519.1 | 1H NMR (400 MHz, METHANOL-d4) δ = 8.17 (br s, 1H), 8.07 (br d, J = 17.2 Hz, 1H), 7.75 (s, 1H), 7.57 (br d, J = 8.8 Hz, 1H), 7.39 (br d, J = 8.8 Hz, 1H), 7.33 (br d, J = 17.2 Hz, 1H), 4.85-4.82 (m, 1H), 4.34-4.27 (m, 2H), 4.06 (br d, J = 14.0 Hz, 1H), 3.98 (br d, J = 4.4 Hz, 2H), 3.86- 3.79 (m, 3H), 3.74 (s, 4H), 3.02 (br t, J = 8.0 Hz, 1H), 2.69 (br d, J = 12.0 Hz, 1H), 2.33 (br t, J = 8.8 Hz, 1H), 2.27 (s, 3H), 2.09-1.98 (m, 4H), 1.49 (br t, J = 6.8 Hz, 3H). |
| 272 | 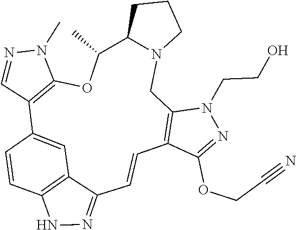 [SMILES: C[C@H]1Oc2c(cnn2C)-c2ccc3[nH]nc(c3c2)/C=C/c2c(OCC#N)nn(CCO)c2CN2CCC[C@H]12] | 515.1 | 1H NMR (400 MHz, METHANOL-d4) δ = 8.51 (br s, 1H), 7.83 (s, 1H), 7.76-7.69 (m, 1H), 7.58 (d, J = 8.8 Hz, 1H), 7.44-7.29 (m, 2H), 5.62 (br s, 1H), 5.24 (d, J = 0.9 Hz, 2H), 5.14 (br d, J = 15.1 Hz, 1H), 4.79-4.69 (m, 1H), 4.38- 4.27 (m, 2H), 4.05 (td, J = 4.1, 11.3 Hz, 2H), 3.99-3.91 (m, 1H), 3.83 (s, 3H), 3.81- 3.73 (m, 1H), 3.67-3.53 (m, 1H), 2.62 (br d, J = 2.6 Hz, 1H), 2.56-2.46 (m, 1H), 2.38 (br s, 1H), 2.30-2.12 (m, 1H), 1.23 (d, J = 6.4 Hz, 3H) |
| 273 | 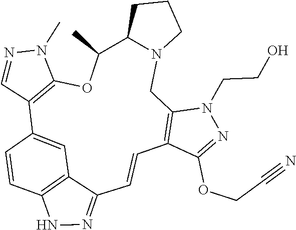 [SMILES: C[C@@H]1Oc2c(cnn2C)-c2ccc3[nH]nc(c3c2)/C=C/c2c(OCC#N)nn(CCO)c2CN2CCC[C@H]12] | 515.2 | 1H NMR (400 MHz, METHANOL-d4) δ = 8.87- 8.56 (m, 1H), 7.91-7.78 (m, 1H), 7.64 (br d, J = 8.8 Hz, 2H), 7.51 (d, J = 8.8 Hz, 1H), 7.26 (br d, J = 16.6 Hz, 1H), 5.21 (s, 2H), 4.83-4.64 (m, 3H), 4.35-4.27 (m, 2H), 4.04- 3.93 (m, 3H), 3.83 (s, 3H), 3.55 (br d, J = 4.8 Hz, 1H), 3.45-3.33 (m, 1H), 2.48-2.31 (m, 1H), 2.14-1.93 (m, 3H), 1.37-1.20 (m, 3H) |
| 274 | 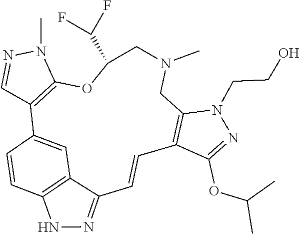 | 528.2 | 1H NMR (400 MHz, DMSO- d6) δ = 12.84 (s, 1H), 8.24 (s, 1H), 8.09 (d, J = 17.2 Hz, 1H), 7.79 (s, 1H), 7.63 (d, J = 8.4 Hz, 1H), 7.49 (d, J = 8.8 Hz, 1H), 7.12 (d, J = 17.2 Hz, 1H), 6.35-5.95 (m, 1H), 4.94-4.86 (m, 2H), 4.84 (t, J = 5.2 Hz, 1H), 4.07-3.99 (m, 2H), 3.99- 3.94 (m, 1H), 3.89-3.84 (m, 1H), 3.71 (s, 3H), 3.67-3.62 (m, 2H), 3.11 (br d, J = 14.4 Hz, 1H), 2.99-2.90 (m, 1H), 2.34 (s, 3H), 1.41-1.37 (m, 6H) |
| 275 | 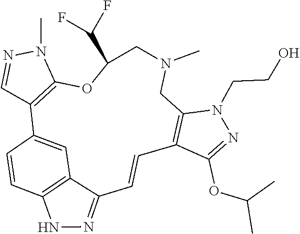 | 528.2 | 1H NMR (400 MHz, DMSO- d6) δ = 12.84 (s, 1H), 8.24 (s, 1H), 8.09 (d, J = 17.2 Hz, 1H), 7.79 (s, 1H), 7.64 (d, J = 8.4 Hz, 1H), 7.49 (d, J = 8.8 Hz, 1H), 7.12 (d, J = 17.2 Hz, 1H), 6.30-5.97 (m, 1H), 4.94-4.87 (m, 2H), 4.86-4.83 (m, 1H), 4.07-3.99 (m, 2H), 3.98-3.94 (m, 1H), 3.89-3.84 (m, 1H), 3.71 (s, 3H), 3.66-3.61 (m, 2H), 3.14-3.08 (m, 1H), 2.98- 2.90 (m, 1H), 2.34 (s, 3H), 1.41-1.37 (m, 6H) |
| 276 | 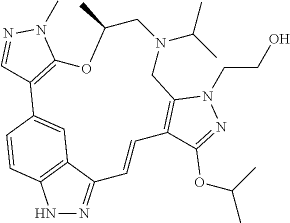 [SMILES: CC(C)Oc1nn(CCO)c2c1/C=C/c1n[nH]c3ccc(cc13)-c1cnn(C)c1O[C@@H](C)CN(C(C)C)C2] | 520.2 | 1H NMR (400 MHz, METHANOL-d4) δ = 8.70 (s, 1H), 8.07 (br d, J = 16.8 Hz, 1H), 7.83 (s, 1H), 7.69-7.64 (m, 1H), 7.48 (d, J = 8.8 Hz, 1H), 7.31 (d, J = 16.8 Hz, 1H), 5.03-4.95 (m, 1H), 4.76 (br s, 1H), 4.19-4.13 (m, 2H), 4.08- 3.97 (m, 2H), 3.85 (t, J = 5.6 Hz, 2H), 3.76 (s, 3H), 2.95- 2.84 (m, 3H), 1.48-1.44 (m, 6H), 1.13 (br d, J = 6.4 Hz, 3H), 1.04 (d, J = 6.4 Hz, 3H), 1.00 (d, J = 6.4 Hz, 3H) |
| 277 | 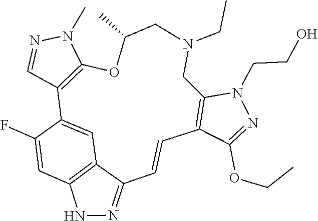 | 510.0 | 1H NMR (400 MHz, METHANOL-d4) δ = 8.75- 8.67 (m, 1H), 8.17-8.08 (m, 1H), 7.86-7.81 (m, 1H), 7.31- 7.21 (m, 2H), 4.73 (br d, J = 1.9 Hz, 1H), 4.34 (q, J = 7.0 Hz, 2H), 4.19-4.09 (m, 2H), 4.01 (br s, 2H), 3.84 (t, J = 5.3 Hz, 2H), 3.77 (s, 3H), 3.04- 2.93 (m, 2H), 2.68-2.59 (m, 2H), 1.49 (t, J = 7.1 Hz, 3H), 1.12 (br d, J = 6.1 Hz, 3H), 0.98 (br t, J = 7.1 Hz, 3H) |
| 278 | 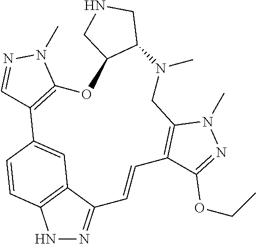 [SMILES: CCOc1nn(C)c2c1/C=C/c1n[nH]c3ccc(cc13)-c1cnn(C)c1O[C@H]1CNC[C@@H]1N(C)C2] | 475.1 | 1H NMR (400 MHz, METHANOL-d4) δ = 8.08 (s, 1H), 7.97 (d, J = 17.2 Hz, 1H), 7.66 (s, 1H), 7.50 (d, J = 8.8 Hz, 1H), 7.35 (d, J = 8.8 Hz, 1H), 7.29 (d, J = 17.2 Hz, 1H), 4.94-4.92 (m, 1H), 4.30-4.21 (m, 2H), 3.97 (br d, J = 14.8 Hz, 1H), 3.69 (s, 3H), 3.67- 3.60 (m, 2H), 3.58 (s, 3H), 3.21-3.12 (m, 1H), 2.88-2.74 (m, 2H), 2.63-2.55 (m, 1H), 2.04 (s, 3H), 1.48 (t, J = 7.2 Hz, 3H) |
| 279 | 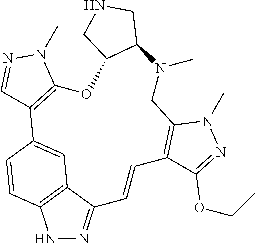 [SMILES: CCOc1nn(C)c2c1/C=C/c1n[nH]c3ccc(cc13)-c1cnn(C)c1O[C@@H]1CNC[C@H]1N(C)C2] | 475.1 | 1H NMR (400 MHz, METHANOL-d4) δ = 8.18 (s, 1H), 8.06 (d, J = 17.2 Hz, 1H), 7.76 (s, 1H), 7.63-7.58 (m, 1H), 7.43 (d, J = 8.8 Hz, 1H), 7.33 (d, J = 17.2 Hz, 1H), 5.07 (br d, J = 2.8 Hz, 1H), 4.34- 4.25 (m, 2H), 4.12 (d, J = 14.8 Hz, 1H), 3.82-3.73 (m, 5H), 3.65 (s, 3H), 3.34 (s, 1H), 3.04- 2.92 (m, 2H), 2.82-2.74 (m, 1H), 2.14 (s, 3H), 1.50 (t, J = 7.2 Hz, 3H) |
| 280 | 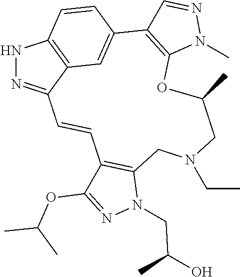 [SMILES: CCN1Cc2c(c(OC(C)C)nn2C[C@H](C)O)/C=C/c2n[nH]c3ccc(cc23)-c2cnn(C)c2O[C@@H](C)C1] | 520.1 | 1H NMR (400 MHz, METHANOL-d4) δ = 8.60 (s, 1H), 8.12 (d, J = 16.8 Hz, 1H), 7.81 (s, 1H), 7.65 (d, J = 8.8 Hz, 1H), 7.46 (d, J = 8.8 Hz, 1H), 7.30 (d, J = 16.8 Hz, 1H), 4.99-4.92 (m, 1H), 4.84 (br s, 1H), 4.09-3.92 (m, 5H), 3.72 (s, 3H), 3.09-3.01 (m, 1H), 2.85 (br d, J = 14.8 Hz, 1H), 2.78-2.61 (m, 2H), 1.43 (t, J = 5.6 Hz, 6H), 1.19 (d, J = 6.0 Hz, 3H), 1.09 (br d, J = 6.4 Hz, 3H), 0.99 (t, J = 7.2 Hz, 3H) |
| 281 | 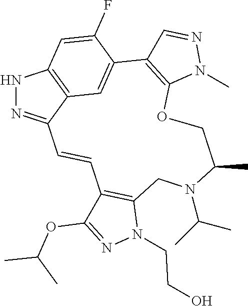 [SMILES: CC(C)Oc1nn(CCO)c2c1/C=C/c1n[nH]c3cc(F)c(cc13)-c1cnn(C)c1OC[C@@H](C)N(C(C)C)C2] | 538.2 | 1H NMR (400 MHz, METHANOL-d4) δ = 8.71 (br d, J = 7.2 Hz, 1H), 7.87-7.82 (m, 1H), 7.66-7.55 (m, 1H), 7.32 (d, J = 16.8 Hz, 1H), 7.21 (d, J = 12.0 Hz, 1H), 5.00 (td, J = 6.0, 12.3 Hz, 1H), 4.50- 4.41 (m, 1H), 4.32-4.23 (m, 1H), 4.12-4.05 (m, 1H), 3.96- 3.83 (m, 5H), 3.80 (s, 3H), 3.78-3.75 (m, 1H), 3.42-3.35 (m, 1H), 1.48-1.40 (m, 9H), 1.16 (br d, J = 6.4 Hz, 3H), 1.01 (d, J = 6.4 Hz, 3H) |
| 282 | 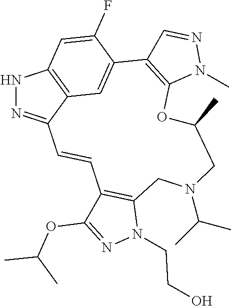 [SMILES: CC(C)Oc1nn(CCO)c2c1/C=C/c1n[nH]c3cc(F)c(cc13)-c1cnn(C)c1O[C@@H](C)CN(C(C)C)C2] | 538.2 | 1H NMR (400 MHz, METHANOL-d4) δ = 8.71 (br d, J = 6.4 Hz, 1H), 8.02 (br d, J = 16.8 Hz, 1H), 7.80 (d, J = 4.4 Hz, 1H), 7.28-7.20 (m, 2H), 4.98 (td, J = 6.0, 12.2 Hz, 1H), 4.61 (br s, 1H), 4.18-4.11 (m, 2H), 4.08-4.01 (m, 2H), 3.85 (t, J = 5.2 Hz, 2H), 3.77 (s, 3H), 2.93 (br s, 3H), 1.45 (dd, J = 3.2, 6.1 Hz, 6H), 1.09 (br d, J = 6.4 Hz, 3H), 0.97 (br dd, J = 6.4, 10.1 Hz, 6H) |
| 283 | 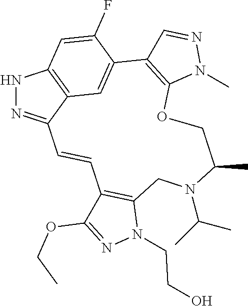 [SMILES: CCOc1nn(CCO)c2c1/C=C/c1n[nH]c3cc(F)c(cc13)-c1cnn(C)c1OC[C@@H](C)N(C(C)C)C2] | 524.2 | 1H NMR (400 MHz, METHANOL-d4) δ = 8.63 (br s, 1H), 7.80 (br d, J = 4.0 Hz, 1H), 7.55 (br d, J = 16.0 Hz, 1H), 7.33-7.24 (m, 1H), 7.17 (br d, J = 12.4 Hz, 1H), 4.43- 4.30 (m, 3H), 4.29-4.19 (m, 1H), 4.08 (td, J = 4.4, 14.0 Hz, 1H), 3.96-3.82 (m, 6H), 3.81- 3.74 (m, 3H), 3.35 (br s, 1H), 3.03 (br s, 1H), 1.49 (t, J = 7.2 Hz, 3H), 1.43-1.36 (m, 3H), 1.13 (br d, J = 6.0 Hz, 3H), 1.01-0.95 (m, 3H) |
| 284 | 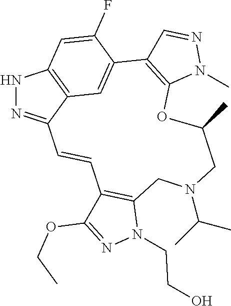 [SMILES: CCOc1nn(CCO)c2c1/C=C/c1n[nH]c3cc(F)c(cc13)-c1cnn(C)c1O[C@@H](C)CN(C(C)C)C2] | 524.1 | 1H NMR (400 MHz, METHANOL-d4) δ = 8.76- 8.62 (m, 1H), 8.00 (br d, J = 17.2 Hz, 1H), 7.78 (br d, J = 4.4 Hz, 1H), 7.23 (br d, J = 6.8 Hz, 1H), 7.19 (s, 1H), 4.60 (br s, 1H), 4.34 (q, J = 7.2 Hz, 2H), 4.21-4.09 (m, 2H), 4.09- 3.98 (m, 2H), 3.86 (br t, J = 5.2 Hz, 2H), 3.76 (s, 3H), 2.96- 2.76 (m, 3H), 1.49 (t, J = 7.0 Hz, 3H), 1.08 (br d, J = 6.1 Hz, 3H), 1.02-0.89 (m, 6H) |
| 285 | 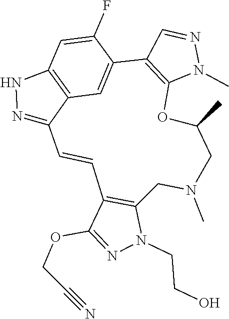 [SMILES: C[C@H]1CN(C)Cc2c(c(OCC#N)nn2CCO)/C=C/c2n[nH]c3cc(F)c(cc23)-c2cnn(C)c2O1] | 507.2 | 1H NMR (400 MHz, DMSO- d6) δ = 12.99-12.85 (m, 1H), 8.64-8.47 (m, 1H), 8.16-8.05 (d, J = 17.2 Hz, 1H), 7.80- 7.68 (m, 1H), 7.42-7.30 (m, 1H), 7.11-6.99 (d, J = 17.2 Hz, 1H), 5.27-5.17 (m, 2H), 4.95-4.65 (m, 2H), 4.12-4.04 (m, 2H), 3.74-3.62 (m, 6H), 3.00 (br d, J = 14.8 Hz, 1H), 2.86-2.77 (m, 1H), 2.69-2.64 (m, 1H), 2.54-2.53 (m, 1H), 2.33-2.30 (m, 3H), 1.07-1.03 (m, 2H) |
| 286 | 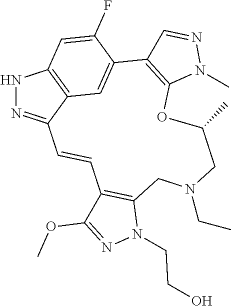 [SMILES: CCN1Cc2c(c(OC)nn2CCO)/C=C/c2n[nH]c3cc(F)c(cc23)-c2cnn(C)c2O[C@H](C)C1] | 496.0 | 1H NMR (400 MHz, DMSO- d6) δ = 13.00-12.73 (m, 1H), 8.65 (br d, J = 6.8 Hz, 1H), 8.04-7.89 (m, 1H), 7.79-7.72 (m, 1H), 7.34 (br d, J = 11.6 Hz, 1H), 7.16-7.05 (m, 1H), 4.70-4.56 (m, 1H), 4.22-4.02 (m, 3H), 3.99 (br s, 2H), 3.94 (br s, 3H), 3.72 (s, 3H), 3.66 (br s, 2H), 3.37-3.30 (m, 2H), 3.04-2.84 (m, 2H), 1.06 (br d, J = 6.0 Hz, 3H), 0.91 (br t, J = 6.4 Hz, 3H) |
| 287 | 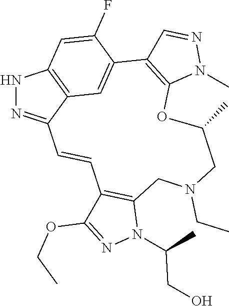 [SMILES: CCOc1nn([C@@H](C)CO)c2c1/C=C/c1n[nH]c3cc(F)c(cc13)-c1cnn(C)c1O[C@H](C)CN(CC)C2] | 524.1 | 1H NMR (400 MHz, METHANOL-d4) δ = 8.78- 8.70 (m, 1H), 8.19-8.11 (m, 1H), 7.86-7.81 (m, 1H), 7.29- 7.25 (m, 1H), 7.25-7.20 (m, 1H), 4.75-4.70 (m, 1H), 4.61 (s, 1H), 4.52-4.46 (m, 1H), 4.36 (q, J = 7.0 Hz, 2H), 4.06- 3.99 (m, 2H), 3.92 (dd, J = 8.1, 11.3 Hz, 1H), 3.77 (s, 3H), 3.76-3.72 (m, 1H), 2.99 (br d, J = 2.9 Hz, 2H), 2.70-2.60 (m, 2H), 1.48 (t, J = 7.1 Hz, 3H), 1.36 (d, J = 6.6 Hz, 3H), 1.13 (br d, J = 6.4 Hz, 3H), 0.99 (t, J = 7.1 Hz, 3H) |
| 288 | 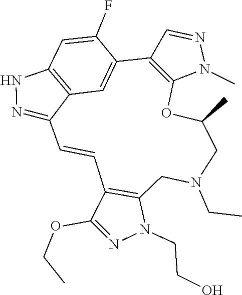 [SMILES: CCOc1nn(CCO)c2c1/C=C/c1n[nH]c3cc(F)c(cc13)-c1cnn(C)c1O[C@@H](C)CN(CC)C2] | 510.1 | 1H NMR (400 MHz, METHANOL-d4) δ = 8.69 (br d, J = 7.2 Hz, 1H), 8.22-7.94 (m, 1H), 7.83 (d, J = 4.8 Hz, 1H), 7.30-7.22 (m, 2H), 4.81- 4.66 (m, 1H), 4.42-4.29 (m, 2H), 4.21-4.11 (m, 2H), 4.10- 3.92 (m, 2H), 3.85 (br t, J = 5.2 Hz, 2H), 3.77 (s, 3H), 3.11- 2.86 (m, 2H), 2.84-2.47 (m, 2H), 1.49 (t, J = 7.0 Hz, 3H), 1.22-0.84 (m, 6H) |
| 289 | 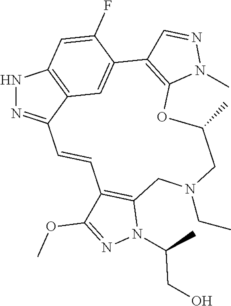 [SMILES: CCN1Cc2c(c(OC)nn2[C@@H](C)CO)/C=C/c2n[nH]c3cc(F)c(cc23)-c2cnn(C)c2O[C@H](C)C1] | 510.3 | 1H NMR (400 MHz, METHANOL-d4) δ = 8.72 (br d, J = 7.2 Hz, 1H), 8.13 (d, J = 17.2 Hz, 1H), 7.83 (d, J = 4.8 Hz, 1H), 7.26-7.16 (m, 2H), 4.77-4.67 (m, 1H), 4.54-4.43 (m, 1H), 4.04-4.02 (m, 3H), 4.02-3.90 (m, 3H), 3.78-3.76 (m, 3H), 3.76-3.72 (m, 1H), 2.99 (br d, J = 4.0 Hz, 2H), 2.72-2.56 (m, 2H), 1.37 (d, J = 6.4 Hz, 3H), 1.13 (d, J = 6.4 Hz, 3H), 0.98 (t, J = 7.2 Hz, 3H) |
| 290 | 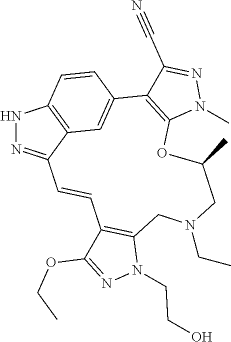 [SMILES: CCOc1nn(CCO)c2c1/C=C/c1n[nH]c3ccc(cc13)-c1c(C#N)nn(C)c1O[C@@H](C)CN(CC)C2] | 517.2 | 1H NMR (400 MHz, DMSO- d6) δ = 13.00 (s, 1H), 8.56 (s, 1H), 7.95 (d, J = 17.2 Hz, 1H), 7.84 (d, J = 8.8 Hz, 1H), 7.63 (d, J = 8.8 Hz, 1H), 7.12 (d, J = 16.8 Hz, 1H), 4.89-4.81 (m, 1H), 4.77-4.67 (m, 1H), 4.33- 4.22 (m, 2H), 4.11-3.99 (m, 2H), 3.97-3.89 (m, 2H), 3.83 (s, 3H), 3.68-3.61 (m, 2H), 2.92 (s, 2H), 2.59-2.53 (m, 2H), 1.45-1.38 (m, 3H), 1.02 (d, J = 6.4 Hz, 3H), 0.92-0.83 (m, 3H) |
| 291 | 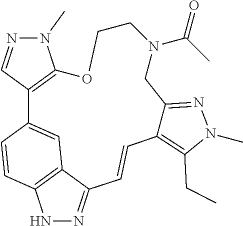 [SMILES: CCc1c2c(nn1C)CN(C(C)=O)CCOc1c(cnn1C)-c1ccc3[nH]nc(c3c1)/C=C/2] | 446.1 | 1H NMR (400 MHz, METHANOL-d4) δ = 8.75- 8.69 (m, 1H), 7.94 (s, 1H), 7.78-7.66 (m, 2H), 7.60-7.46 (m, 1H), 7.17-7.07 (m, 1H), 4.60 (s, 2H), 4.36 (br t, J = 6.8 Hz, 2H), 3.92-3.84 (m, 5H), 3.80 (s, 3H), 2.99 (s, 1H), 2.54- 2.47 (m, 3H), 1.40-1.32 (m, 3H) |
| 292 | 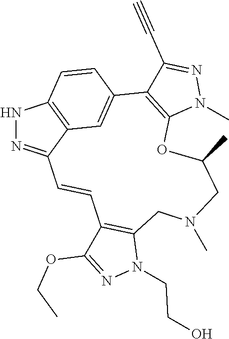 [SMILES: C#Cc1nn(C)c2c1-c1ccc3[nH]nc(c3c1)/C=C/c1c(OCC)nn(CCO)c1CN(C)C[C@H](C)O2] | 502.1 | 1H NMR (400 MHz, METHANOL-d4) δ = 8.53 (s, 1H), 8.28 (dd, J = 1.2, 8.8 Hz, 1H), 8.22-8.08 (m, 1H), 7.50 (d, J = 8.8 Hz, 1H), 7.28 (d, J = 16.8 Hz, 1H), 4.82 (br d, J = 1.6 Hz, 1H), 4.35 (dq, J = 2.8, 7.2 Hz, 2H), 4.19-4.08 (m, 2H), 3.98 (br dd, J = 2.4, 4.2 Hz, 2H), 3.87-3.82 (m, 2H), 3.80 (s, 1H), 3.76 (s, 3H), 3.09- 2.81 (m, 2H), 2.45 (br s, 3H), 1.49 (t, J = 7.2 Hz, 3H), 1.08 (d, J = 6.4 Hz, 3H) |
| 293 | 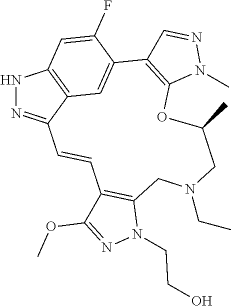 [SMILES: CCN1Cc2c(c(OC)nn2CCO)/C=C/c2n[nH]c3cc(F)c(cc23)-c2cnn(C)c2O[C@@H](C)C1] | 496.1 | 1H NMR (400 MHz, METHANOL-d4) δ = 8.70 (br d, J = 7.2 Hz, 1H), 8.20-7.95 (m, 1H), 7.84 (d, J = 4.8 Hz, 1H), 7.28-7.18 (m, 2H), 4.82- 4.64 (m, 1H), 4.23-4.11 (m, 2H), 4.03 (s, 3H), 3.86 (br t, J = 5.2 Hz, 2H), 3.77 (s, 3H), 3.30 (br s, 2H), 3.11-2.86 (m, 2H), 2.85-2.49 (m, 2H), 1.13 (br d, J = 6.4 Hz, 3H), 1.09- 0.81 (m, 3H) |
| 294 | 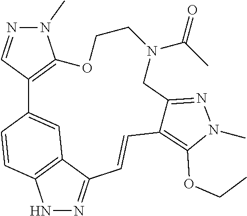 [SMILES: CCOc1c2c(nn1C)CN(C(C)=O)CCOc1c(cnn1C)-c1ccc3[nH]nc(c3c1)/C=C/2] | 462.1 | 1H NMR (400 MHz, METHANOL-d4) δ = 8.62 (s, 1H), 7.86 (s, 1H), 7.68 (d, J = 8.8 Hz, 1H), 7.60-7.47 (m, 2H), 7.25 (d, J = 16.8 Hz, 1H), 4.67 (br s, 2H), 4.36 (br t, J = 7.2 Hz, 2H), 4.29 (q, J = 7.2 Hz., 2H), 3.90 (br t, J = 7.6 Hz, 2H), 3.80 (s, 3H), 3.76 (s, 3H), 2.48 (s, 3H), 1.54 (t, J = 7.2 Hz, 3H) |
| 295 | 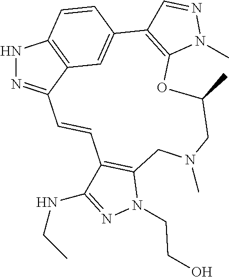 [SMILES: CCNc1nn(CCO)c2c1/C=C/c1n[nH]c3ccc(cc13)-c1cnn(C)c1O[C@@H](C)CN(C)C2] | 477.1 | 1H NMR (400 MHz, METHANOL-d4) δ = 8.53 (s, 1H), 8.38 (d, J = 17.6 Hz, 1H), 7.80 (s, 1H), 7.66 (dd, J = 1.6, 8.8 Hz, 1H), 7.49 (d, J = 8.8 Hz, 1H), 6.92 (d, J = 17.6 Hz, 1H), 4.95- 4.90 (m, 1H), 4.20-4.07 (m, 2H), 3.98-3.86 (m, 2H), 3.85- 3.80 (m, 2H), 3.75-3.71 (m, 3H), 3.38-3.32 (m, 2H), 2.95- 2.86 (m, 2H), 2.44-2.40 (m, 3H), 1.31 (t, J = 7.6 Hz, 3H), 1.08 (d, J = 6.4 Hz, 3H) |
| 296 | 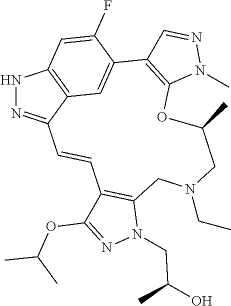 [SMILES: CCN1Cc2c(c(OC(C)C)nn2C[C@H](C)O)/C=C/c2n[nH]c3cc(F)c(cc23)-c2cnn(C)c2O[C@@H](C)C1] | 538.1 | 1H NMR (400 MHz, METHANOL-d4) δ = 8.65 (br d, J = 7.2 Hz, 1H), 8.06 (d, J = 16.8 Hz, 1H), 7.80 (d, J = 4.8 Hz, 1H), 7.29-7.19 (m, 2H), 5.03-4.92 (m, 1H), 4.75-4.65 (m, 1H), 4.14-4.00 (m, 2H), 3.99-3.91 (m, 3H), 3.75 (s, 3H), 3.07-2.85 (m, 2H), 2.69- 2.51 (m, 2H), 1.48-1.42 (m, 6H), 1.21 (d, J = 6.0 Hz, 3H), 1.08 (d, J = 6.4 Hz, 3H), 0.96 (t, J = 7.2 Hz, 3H) |
| 297 | 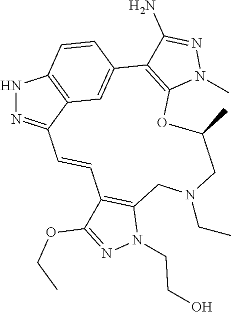 [SMILES: CCOc1nn(CCO)c2c1/C=C/c1n[nH]c3ccc(cc13)-c1c(N)nn(C)c1O[C@@H](C)CN(CC)C2] | 507.2 | 1H NMR (400 MHz, DMSO- d6) δ = 12.72 (s, 1H), 8.39 (s, 1H), 7.95 (d, J = 17.2 Hz, 1H), 7.74 (d, J = 9.6 Hz, 1H), 7.44 (d, J = 8.8 Hz, 1H), 7.09 (d, J = 17.2 Hz, 1H), 4.92-4.75 (m, 1H), 4.55 (s, 2H), 4.54-4.49 (m, 1H), 4.32-4.21 (m, 2H), 4.11-3.97 (m, 2H), 3.92 (s, 2H), 3.65 (s, 2H), 3.48 (s, 3H), 2.86 (s, 2H), 2.49-2.43 (m, 2H), 1.47-1.35 (m, 3H), 0.98 (d, J = 6.4 Hz, 3H), 0.92-0.82 (m, 3H) |
| 298 | 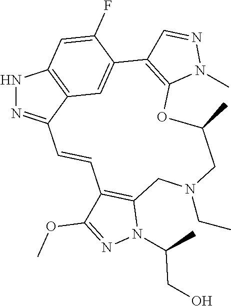 [SMILES: CCN1Cc2c(c(OC)nn2[C@@H](C)CO)/C=C/c2n[nH]c3cc(F)c(cc23)-c2cnn(C)c2O[C@@H](C)C1] | 510.2 | 1H NMR (400 MHz, METHANOL-d4) δ = 8.67 (br d, J = 7.2 Hz, 1H), 8.04-7.87 (m, 1H), 7.83 (d, J = 4.8 Hz, 1H), 7.28-7.18 (m, 2H), 4.56- 4.43 (m, 1H), 4.20-4.06 (m, 2H), 4.03 (s, 3H), 3.88-3.78 (m, 2H), 3.77 (s, 3H), 3.72- 3.66 (m, 1H), 3.16-2.91 (m, 2H), 2.89-2.62 (m, 2H), 1.44 (br d, J = 6.4 Hz, 3H), 1.11 (br d, J = 6.2 Hz, 6H) |
| 299 | 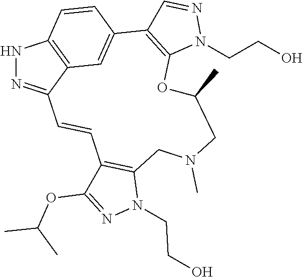 [SMILES: CC(C)Oc1nn(CCO)c2c1/C=C/c1n[nH]c3ccc(cc13)-c1cnn(CCO)c1O[C@@H](C)CN(C)C2] | 522.2 | 1H NMR (400 MHz, METHANOL-d4) δ = 8.54 (s, 1H), 8.30 (d, J = 17.2 Hz, 1H), 7.85 (s, 1H), 7.68 (d, J = 8.8 Hz, 1H), 7.48 (d, J = 8.8 Hz, 1H), 7.30 (d, J = 17.2 Hz, 1H), 5.02-4.94 (m, 2H), 4.31-4.18 (m, 1H), 4.17-4.04 (m, 3H), 4.00-3.88 (m, 4H), 3.87-3.78 (m, 2H), 3.01-2.81 (m, 2H), 2.46 (s, 3H), 1.44 (t, J = 6.0 Hz, 6H), 1.12 (d, J = 6.0 Hz, 3H) |

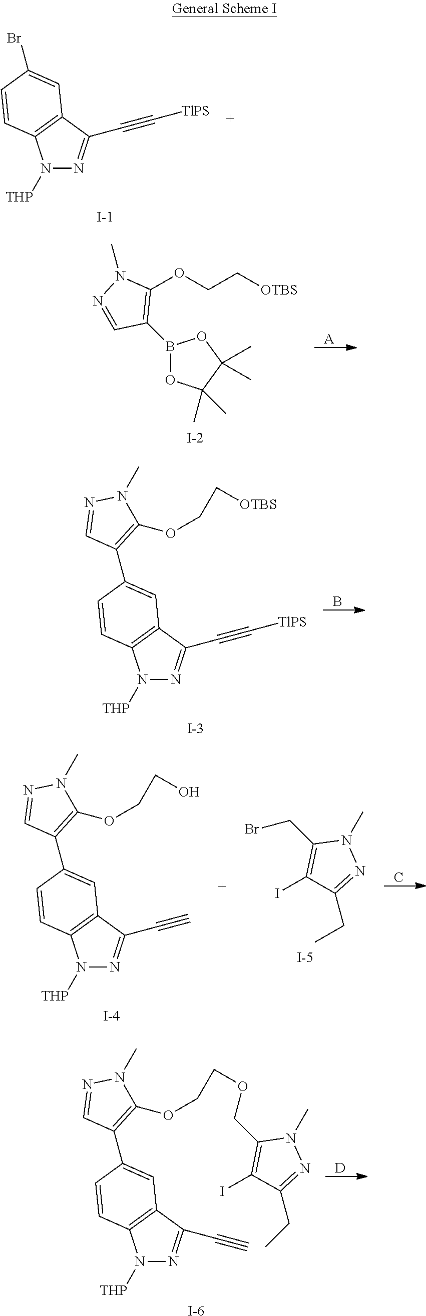

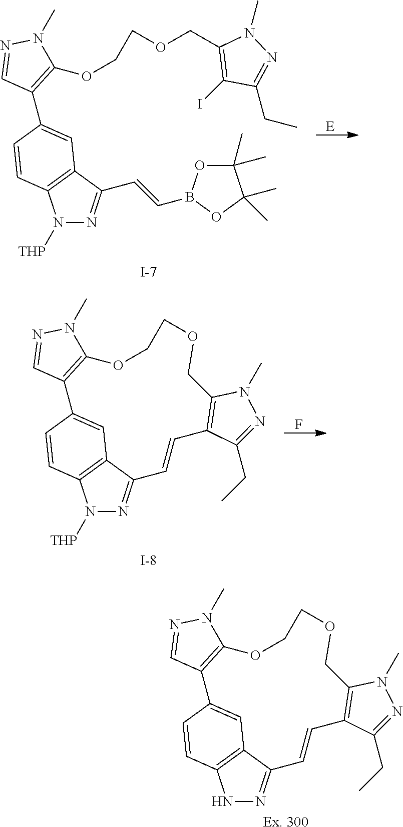

General Scheme I is using Example 300 as an illustration. Compounds I-1 and I-2 are prepared via conventional chemistry from commercially available materials. Under palladium catalyzed coupling condition A, compounds I-1 and I-2 are converted to a product, I-3, which is then deprotected under condition B to generate 1-4. I-4 is alkylated with I-5 under condition C to produce a product, e. g. I-6. A boronic ester I-7 is produced under Condition D followed by palladium-catalyzed Suzuki reaction under Condition E to generate the macrocyclic product, e. g. I-8. After deprotection, I-8 is converted to the final product, e. g. Ex. 300.

### Preparation of 2-(5-bromo-1-tetrahydropyran-2-yl-indazol-3-yl)ethynyl-triisopropyl-silane (I-1-300)

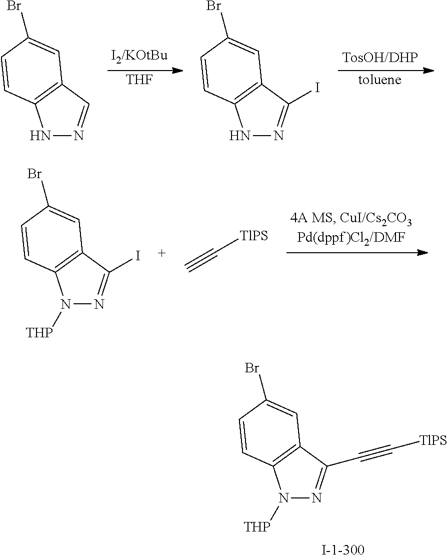

Step 1. A solution of 5-bromo-1H-indazole (21.0 g, 107 mmol, 1 eq) in THF (250 mL) was cooled down on an ice bath and KOtBu (35.9 g, 320 mmol, 3 eq) was added portion wise. The resulting slurry was stirred at 0° C. and a solution of I₂ (54.1 g, 213 mmol, 42.9 mL, 2 eq) in THF (250 mL) was added dropwise. The mixture was stirred at 25° C. for 12 hours. On completion, the reaction mixture was filtered and the filtrate was diluted with H₂O (20 mL) and extracted with EtOAc (20 mL*3). The combined organic layers were washed with brine (20 mL*3), dried over Na₂SO₄, filtered and concentrated under reduced pressure. The residue was purified by flash silica gel chromatography to afford 5-bromo-3-iodo-1H-indazole (120 g, 350 mmol, 82% yield, 94% purity) as a white solid. LCMS: 324.7 (M+1).

Step 2. To a mixture of 5-bromo-3-iodo-1H-indazole (25.0 g, 77.4 mmol, 1 eq) and 3,4-dihydro-2H-pyran (13.0 g, 155 mmol, 2 eq) in toluene (250 mL) was added 4-methylbenzenesulfonic acid (2.67 g, 15.5 mmol, 0.2 eq). The mixture was stirred at 90° C. for 12 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford 5-bromo-3-iodo-1-tetrahydropyran-2-yl-indazole (24.0 g, 58.9 mmol, 76% yield) as a white solid. ¹H NMR (400 MHz, CDCl₃) δ=7.64 (d, J=1.6 Hz, 1H), 7.54-7.44 (m, 2H), 5.68 (dd, J=3.2, 9.1 Hz, 1H), 4.04-3.95 (m, 1H), 3.79-3.66 (m, 1H), 2.58-2.46 (m, 1H), 2.20-2.03 (m, 2H), 1.87-1.54 (m, 3H).

Step 3. To a mixture of 5-bromo-3-iodo-1-tetrahydropyran-2-yl-indazole (23.0 g, 56.5 mmol, 1 eq) and ethynyl(triisopropyl)silane (11.3 g, 62.2 mmol, 1.1 eq) in DMF (250 mL) was added Cs₂CO₃ (55.2 g, 170 mmol, 3 eq), Pd(dppf)Cl₂ (2.48 g, 3.39 mmol, 0.06 eq) and CuI (646 mg, 3.39 mmol, 0.06 eq) under N₂. The mixture was stirred at 25° C. for 3 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford compound 2-(5-bromo-1-tetrahydropyran-2-yl-indazol-3-yl)ethynyl-triisopropyl-silane (I-1-300, 38.0 g, 79.9 mmol, 70% yield) as a white solid. ¹H NMR (400 MHz, CDCl₃) δ=7.87 (s, 1H), 7.57-7.43 (m, 2H), 5.70 (dd, J=2.4, 9.2 Hz, 1H), 4.02 (bd, J=11.2 Hz, 1H), 3.80-3.65 (m, 1H), 2.59-2.41 (m, 1H), 2.14 (d, J=3.2 Hz, 1H), 2.08 (s, 1H), 1.79-1.70 (m, 2H), 1.67 (s, 1H), 1.22-1.18 (m, 18H), 1.18-1.14 (m, 3H).

### Preparation of tert-butyl-dimethyl-[2-[2-methyl-4-(4,4,5,5-tetramethyl-1,3,2-dioxaborolan-2-yl) pyrazol-3-yl]oxyethoxy]silane (I-2-300)

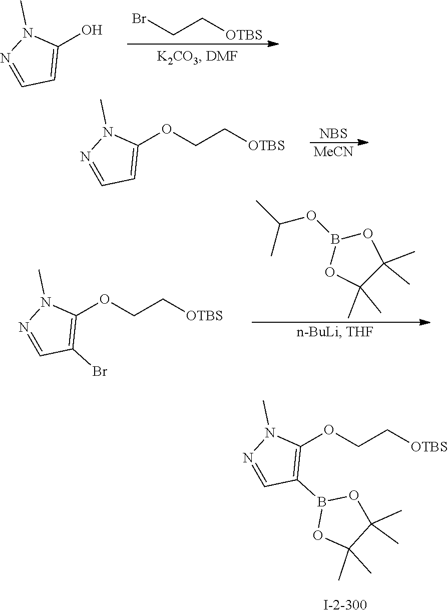

Step 1. To a mixture of 2-methylpyrazol-3-ol (15.0 g, 152 mmol, 1 eq) in DMF (150 mL) was added 2-bromoethoxy-tert-butyl-dimethyl-silane (40.2 g, 168 mmol, 1.1 eq) and K₂CO₃ (63.4 g, 458 mmol, 3 eq), the reaction mixture was stirred at 30° C. for 12 hours. On completion, the reaction mixture was diluted with water (150 mL) and extracted with EA (2×200 mL). The combined organic layers were dried over Na₂SO₄, filtered and concentrated in vacuo to afford tert- butyl- dimethyl-[2-(2- methylpyrazol-3-yl) oxyethoxy]silane (9.00 g, 35.1 mmol, 22% yield) as colorless oil. ¹H NMR (400 MHz, DMSO-d₆) δ 7.19 (d, J=2.0 Hz, 1H), 5.62 (d, J=2.0 Hz, 1H), 4.10-4.07 (m, 2H), 3.91-3.88 (m, 2H), 3.53 (s, 3H), 0.86 (s, 9H), 0.08-0.02 (m, 6H).

Step 2. To a mixture of tert-butyl-dimethyl-12-(2-methylpyrazol-3-yl)oxyethoxy]silane (8.00 g, 31.2 mmol, 1 eq) in ACN (70 mL) at 0° C. and then the reaction mixture was added NBS (6.11 g, 34.3 mmol, 1.1 eq), the reaction mixture was stirred at 25° C. for 2 hours. On completion, the reaction mixture was diluted with water (100 mL) and extracted with EA (2×100 mL). The combined organic layers was dried over Na₂SO₄, filtered and concentrated in vacuo to afford 2-(4-bromo-2-methyl-pyrazol-3-yl)oxyethanol (9.00 g, 90% yield) as yellow oil. LCMS: [M+H]+m/z=220.9

Step 3. To a mixture of 2-(4-bromo-2-methyl-pyrazol-3-yl)oxyethoxy-tert-butyl-dimethyl-silane (18.0 g, 53.6 mmol, 1 eq) in THF (180 mL) was added n-BuLi (2.5 M, 64.4 mL, 3 eq), the reaction mixture was stirred at −60° C. for 0.5 hours, and then 2-isopropoxy-4,4,5,5-tetramethyl -1,3,2-dioxaborolane (29.9 g, 161 mmol, 32.8 mL, 3 eq) was added, the reaction mixture was stirred at −60° C. for 2 hours. On completion, the reaction mixture was quenched with aq·NH₄Cl (300 mL) under stirring, and then the residue was diluted with water (200 mL) and extracted with EA (2×200 mL). The combined organic layers were dried over Na₂SO₄, filtered and concentrated in vacuo to give a residue. The residue was purified by column chromatography to afford tert-butyl-dimethyl-[2-[2-methyl-4-(4,4,5,5-tetramethyl-1,3,2-dioxaborolan-2-yl) pyrazol-3-yl]oxyethoxy]silane (14.2 g, 37.3 mmol, 69% yield) as yellow oil. LCMS: [M+H]⁺ m/z=383.4

### General Method Z. Preparation of (17E)-16-ethyl-8,14-dimethyl-2,8,10,11,13,14-hexahydro-3,5-etheno[1,4]dioxacyclopentadecino[10,11-c:15,14-c′:6,7-c″]tripyrazole (Ex. 300)

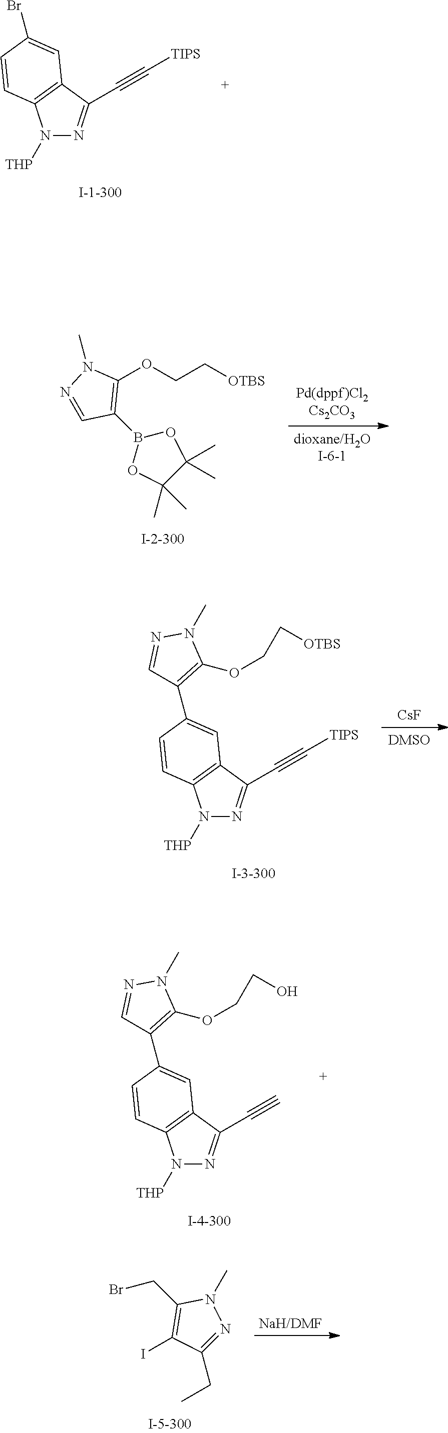

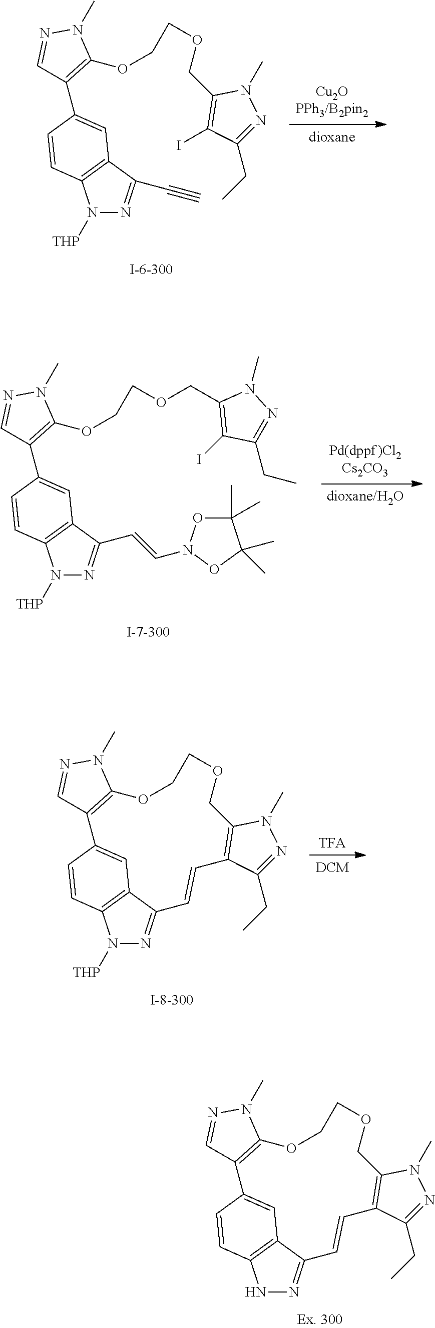

Step 1. To a mixture of I-2-300 (5.19 g, 13.5 mmol, 1.1 eq) in dioxane (60 mL) and H₂O (12 ml) was added I-1-300 (5.70 g, 12.3 mmol, 1 eq), Cs₂CO₃ (12.0 g, 37.0 mmol, 3 eq) and Pd(dppf)Cl₂ (451 mg, 0.617 mmol, 0.05 eq), the reaction mixture was stirred at 90° C. for 12 hours. On completion, the reaction mixture was diluted with water (80 mL) and extracted with EA (2×80 mL). The combined organic layers were dried over Na₂SO₄, filtered and concentrated in vacuo to give a residue. The residue was purified by column chromatography to afford I-3-300 (4.83 g, 7.58 mmol, 61% yield) as a yellow solid. LCMS: [M+H]⁺ m/z=638.0

Step 2. To a mixture of I-3-300 (4.00 g, 6.28 mmol, 1 eq) in DMSO (40 mL) was added CsF (2.86 g, 18.8 mmol, 3 eq), and the reaction mixture was stirred at 25° C. for 12 hours. On completion, the reaction mixture was diluted with water (50 mL) and extracted with EA (2×50 mL). The combined organic layers were dried over Na₂SO₄, filtered and concentrated in vacuo to afford I-4-300 (2.10 g, 5.73 mmol, 91.2% yield) as yellow oil. LCMS: [M+H]⁺ m/z=367.2

Step 3. To a mixture of I-4-300 (100 mg, 0.272 mmol, 1 eq) in DMF (3 ml) was added NaH (27.2 mg, 0.682 mmol, 60% purity, 2.5 eq) at 0° C. The reaction mixture was stirred at 0° C. for 0.5 hour, and then I-5-300 (89.7 mg, 0.272 mmol, 1 eq) was added to the reaction mixture. The reaction mixture was stirred at 25° C. for 12 hours. On completion, the reaction mixture was quenched with water (0.5 mL), and then the residue was diluted with water (5 mL) and extracted with EA (2×5 mL). The combined organic layers were dried over Na₂SO₄, filtered and concentrated in vacuo. The residue was purified by column chromatography to afford I-6-300 (50.0 mg, 0.081 mmol, 29% yield) as yellow oil. ¹H NMR (400 MHz, DMSO-d₆) δ 7.83 (s, 1H), 7.79 (s, 1H), 7.74 (s, 2H), 5.87 (dd, J=2.4, 9.6 Hz, 1H), 4.58 (s, 1H), 4.56 (s, 2H), 4.10-4.06 (m, 2H), 3.89 (d, J=11.2 Hz, 1H), 3.80 (s, 3H), 3.79-3.72 (m, 1H), 3.68 (s, 3H), 3.66 (s, 2H), 2.47-2.44 (m, 1H), 2.36-2.29 (m, 1H), 2.18 (s, 1H), 2.08-1.99 (m, 2H), 1.82-1.70 (m, 1H), 1.60 (d, J=3.6 Hz, 2H), 1.13 (t, J=7.6 Hz, 3H).

Step 4. To a mixture of I-6-300 (430 mg, 0.699 mmol, 1 eq) in dioxane (5 mL) was added Cu₂O (50.0 mg, 0.349 mmol, 0.5 eq), PPh₃ (201 mg, 0.769 mmol, 1.1 eq) and 4,4,5,5-tetramethyl-2-(4,4,5,5-tetramethyl-1,3,2-dioxaborolan-2-yl)-1,3,2- dioxaborolane (355 mg, 1.40 mmol, 2 eq). The reaction mixture was stirred at 80° C. for 12 hours. On completion, the residue was diluted with water (10 mL) and extracted with EA (2×10 mL). The combined organic layers were dried over Na₂SO₄, filtered and concentrated in vacuo. The residue was purified by column chromatography to afford I-7-300 (384 mg, 0.517 mmol, 73% yield) as yellow oil. LCMS: [M+H]⁺ m/z=742.8

Step 5. To a mixture of I-7-300 (350 mg, 0.471 mmol, 1 eq) in dioxane (5 mL) and H₂O (1 mL) was added Pd(dppf)Cl₂ (34.4 mg, 0.047 mmol, 0.1 eq) and Cs₂CO₃ (460 mg, 1.41 mmol, 3 eq). The reaction mixture was stirred at 90° C. for 12 hours. On completion, the reaction mixture was diluted with water (10 mL) and extracted with EA (2×10 mL). The combined organic layers were dried over Na₂SO₄, filtered and concentrated in vacuo. The residue was purified by column chromatography to afford I-8-300 (188 mg, 0.384 mmol, 81% yield) as yellow oil. ¹H NMR (400 MHz, DMSO-d₆) δ 8.53 (s, 1H), 8.05 (d, J=17.6 Hz, 1H), 7.95 (s, 1H), 7.79-7.76 (m, 1H), 7.72-7.69 (m, 1H), 6.88 (s, 1H), 5.81 (dd, J=2.4, 9.6 Hz, 1H), 4.87 (s, 2H), 4.29 (s, 2H), 4.04-4.00 (m, 2H), 3.94-3.88 (m, 1H), 3.77 (d, J=8.8 Hz, 1H), 3.74 (s, 3H), 3.72 (s, 3H), 2.81 (q, J=7.6 Hz, 2H), 1.94-1.86 (m, 4H), 1.58 (d, J=3.6 Hz, 2H), 1.27 (s, 3H).

Step 6. To a mixture of I-8-300 (168 mg, 0.343 mmol, 1 eq) in DCM (1 mL) was added TFA (12.9 g, 113 mmol, 8.40 mL, 329 eq), the reaction mixture was stirred at 25° C. for 1 hour. On completion, the reaction mixture was concentrated in vacuo. The residue was triturated with EA (3 mL), filtered and concentrated in vacuo to afford Ex. 3001 (38.7 mg, 0.956 mmol, 27% yield) as brown solid. ¹H NMR (400 MHz, DMSO-d₆) δ 8.54 (s, 1H), 8.02 (d, J=17.6 Hz, 1H), 7.92 (s, 1H), 7.70 (dd, J=1.2, 8.8 Hz, 1H), 7.49 (d, J=8.8 Hz, 1H), 6.90 (d, J=17.6 Hz, 1H), 4.87 (s, 2H), 4.30 (dd, J=2.4, 4.4 Hz, 2H), 4.05-3.99 (m, 2H), 3.74 (s, 3H), 3.72 (s, 3H), 2.80 (q, J=7.2 Hz, 2H), 1.27 (t, J=7.2 Hz, 3H). LCMS: [M+H]⁺ m/z=404.8.

### Preparation of methyl 2-[5-(bromomethyl)-4-iodo-3-methyl-pyrazol-1-yl]acetate (I-1-301)

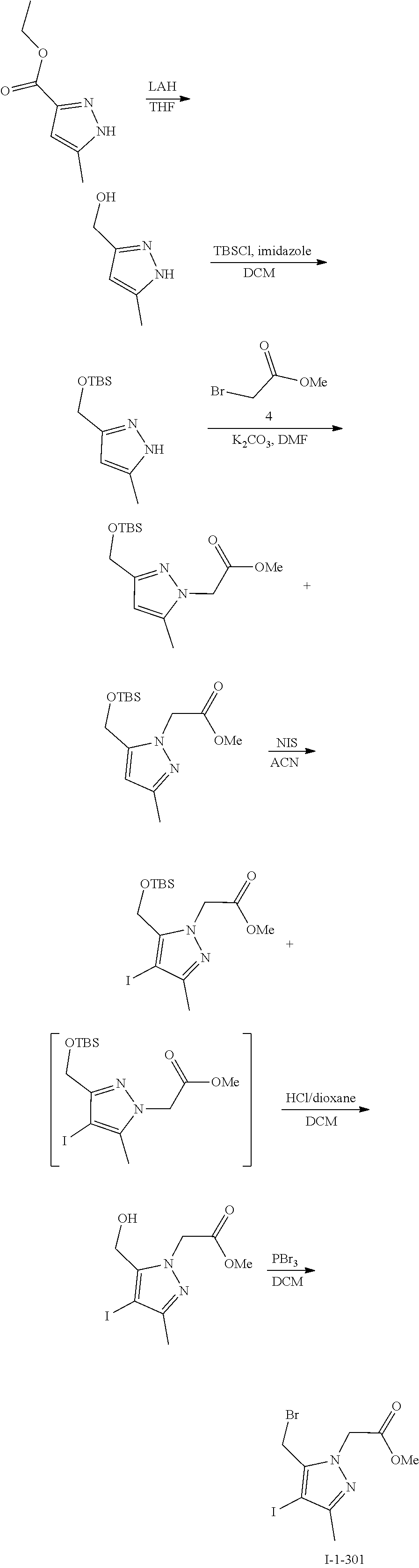

Step 1. To a mixture of ethyl 5-methyl-1H-pyrazole-3-carboxylate (10.0 g, 64.8 mmol, 1 eq) in THF (100 mL) was added LAH (2.95 g, 77.8 mmol, 1.2 eq), the reaction mixture was stirred at 0° C. for 2 hours. On completion, the mixture was slowly added to water (3 ml), saturated sodium hydroxide (3 ml) and water (9 ml) to quench. The reaction mixture was filtered and concentrated under reduced pressure to afford (5-methyl-1H-pyrazol-3-yl)methanol (3.40 g, 30.3 mmol, 46% yield) as yellow solid. ¹H NMR (400 MHz, DMSO-d₆) δ 5.89 (s, 1H), 4.36 (s, 2H), 2.16 (s, 3H)

Step 2. To a mixture of (5-methyl-1H-pyrazol-3-yl)methanol (25.0 g, 222 mmol, 1 eq) in DCM (300 mL) was added imidazole (30.3 g, 445 mmol, 2 eq) and TBSCl (50.4 g, 334 mmol, 40.9 mL, 1.5 eq) at 0° C., the reaction mixture was stirred at 25° C. for 12 hours. On completion, the residue was diluted with water (300 mL) and extracted with DCM (2×300 mL). The combined organic layers was dried over Na₂SO₄, filtered and concentrated in vacuo to afford tert-butyl-dimethyl-[(5-methyl-1H-pyrazol-3-yl)methoxy]silane (65.0 g, 90% yield) as a yellow oil. ¹H NMR (400 MHz, DMSO-d6) δ 12.34-12.09 (m, 1H), 5.90 (s, 1H), 4.55 (s, 2H), 2.17 (s, 3H), 0.86 (s, 9H), 0.05-0.02 (m, 6H).

Step 3. A mixture of methyl 2-bromoacetate (40.5 g, 265 mmol, 25.0 mL, 2 eq), tert-butyl-dimethyl-[(5-methyl-1H-pyrazol-3-yl)methoxy]silane (30.0 g, 132 mmol, 1 eq), K₂CO₃ (54.9 g, 397 mmol, 3 eq) was combined in DMF (300 mL). The mixture was stirred at 80° C. for 16 hours. On completion, the reaction mixture was filtered and then the residue was diluted with water (500 mL) and extracted with EA (2×500 mL). The combined organic layers was dried over Na₂SO₄, filtered and concentrated in vacuo to give a residue. The residue was purified by column chromatography to afford methyl 2-[3-[[tert-butyl(dimethyl)silyl]oxymethyl]-5- methyl-pyrazol-1-yl]acetate and methyl 2-[5-[[tert-butyl (dimethyl)silyl]oxymethyl]-3- methyl-pyrazol-1-yl]acetate (21.2 g, 35.5 mmol, 52% yield) as yellow oil. ¹H NMR (400 MHz, DMSO-d6) δ 5.98 (s, 1H), 4.94 (s, 2H), 4.91 (s, 2H), 3.65 (s, 3H), 2.17 (s, 3H), 0.86 (s, 9H), 0.04 (s, 6H).

Step 4. To a mixture of methyl 2-[5-[[tert-butyl(dimethyl)silyl]oxymethyl]-3-methyl -pyrazol-1-yl]acetate and methyl 2-[3-[[tert-butyl (dimethyl) silyl]oxymethyl]-5-methyl-pyrazol-1-yl]acetate (21.2 g, 35.5 mmol, 1 eq) in ACN (200 mL) was added NIS (7.99 g, 35.5 mmol, 1 eq), the reaction mixture was stirred at 25° C. for 12 hours. On completion, the residue was diluted with water (200 mL) and extracted with EA (2×200 mL). The combined organic layers was dried over Na₂SO₄, filtered and concentrated in vacuo to give a residue. The residue was purified by column chromatography to afford methyl 2-[3-[[tert-butyl(dimethyl) silyl]oxymethyl]-4-iodo-5- methyl-pyrazol-1-yl]acetate (6.19 g, 14.5 mmol, 41% yield) as yellow solid and afford methyl 2-[5-[[tert-butyl (dimethyl) silyl]oxymethyl]-4-iodo-3-methyl -pyrazol-1-yl]acetate (13.0 g, 30.6 mmol, 86% yield) yellow oil. ¹H NMR (400 MHz, DMSO-d6) δ 5.09 (s, 2H), 4.48 (s, 2H), 3.68 (s, 3H), 2.20 (s, 3H), 0.87 (s, 9H), 0.06 (s, 6H). and 1H NMR (400 MHz, DMSO-d6) δ 5.03 (s, 2H), 4.64 (s, 2H), 3.65 (s, 3H), 2.11 (s, 3H), 0.85 (s, 9H), 0.06 (s, 6H).

Step 5. To a mixture of methyl 2-|5-∥tert-butyl(dimethyl)silyl|oxymethyl|-4-iodo-3-methyl -pyrazol-1-yl]acetate (10.0 g, 23.5 mmol, 1 eq) in DCM (100 mL) was added HCl/dioxane (4 M, 10.0 mL, 1.70 eq), the reaction mixture was stirred at 25° C. for 12 hours. On completion, the reaction mixture was concentrated in vacuo, and then the residue was diluted with water (100 mL) and extracted with DCM (2×100 mL). The combined organic layers was dried over Na₂SO₄, filtered and concentrated in vacuo to afford methyl 2-[5-(hydroxymethyl)-4-iodo-3-methyl - pyrazol-1-yl]acetate (9.78 g, 92% yield) as yellow solid. ¹H NMR (400 MHz, DMSO-d6) δ 5.07-5.03 (m, 2H), 4.43-4.41 (m, 2H), 3.67-3.65 (m, 3H), 2.11-2.09 (m, 3H), 0.84 (s, 5H), −0.04 (s, 4H)

Step 6. To a solution of methyl 2-[5-(hydroxymethyl)-4-iodo-3-methyl-pyrazol-1-yl]acetate (5.00 g, 16.1 mmol, 1 eq) in DCM (90 mL). Then PBr₃ (4.36 g, 16.1 mmol, 1 eq) was added dropwise at 0° C. The mixture was stirred at 25° C. for 1 hour. On completion, the residue was diluted with water (100 mL) and extracted with DCM (2×100 mL). The combined organic layers was dried over Na₂SO₄, filtered and concentrated in vacuo to give a residue. The residue was purified by column chromatography and then purified by reverse phase (0.1% FA condition) to afford I-1-301 (4.50 g, 12.0 mmol, 74% yield) as white solid. 1H NMR (400 MHz, DMSO-d6) δ 5.13 (s, 2H), 4.70 (s, 2H), 3.67 (s, 3H), 2.11 (s, 3H).

### General Method B. Preparation of 2-[(17E)-8,16-dimethyl-2,10,11,13-tetrahydro-3,5-etheno[1,4]dioxacyclopentadecino[10,11-c:15,14-c′:6,7-c′]tripyrazol-14(8H)-yl]-N-ethylacetamide (Ex. 301)

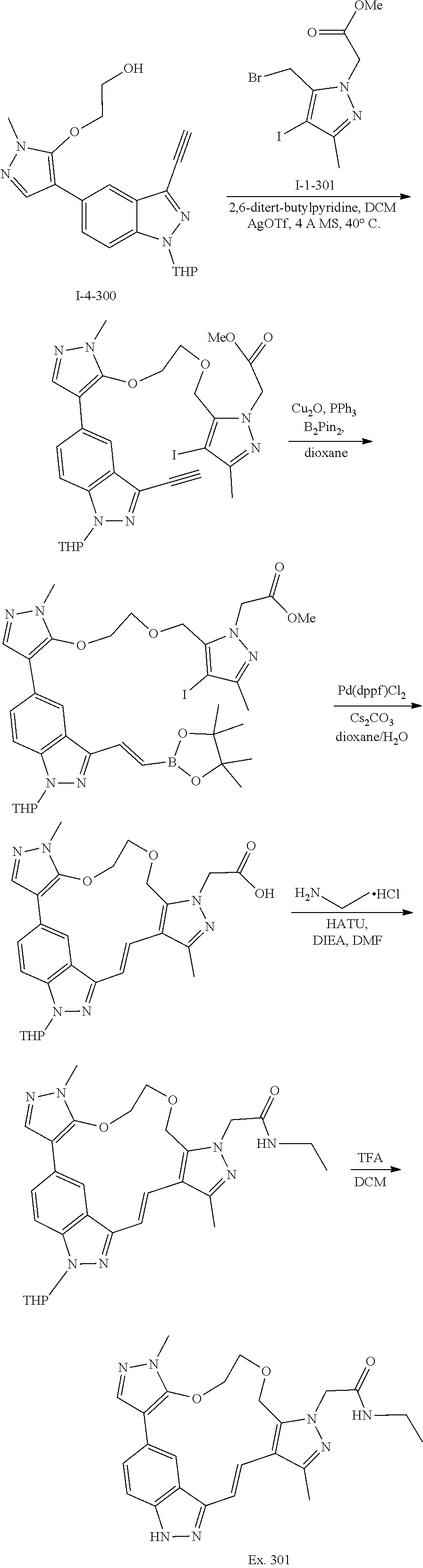

Step 1. To a mixture of I-4-300 (2.70 g, 7.37 mmol, 1 eq) in DCM (10 mL) was added I-1-2 (2.02 g, 5.42 mmol, 7.36^{e-1} eq), AgOTf (3.79 g, 14.7 mmol, 2 eq), 4A MS (7.37 mmol, 1 eq) and 2,6-ditert-butylpyridine (8.46 g, 44.2 mmol, 6 eq), the reaction mixture was stirred at 40° C. for 12 hours. On completion, the residue was diluted with water (10 mL) and extracted with DCM (2×10 mL). The combined organic layer was dried over Na₂SO₄, filtered and concentrated in vacuo to give a residue. The residue was purified by flash column chromatography to afford methyl 2-[5-[2-[4-(3-ethynyl-1-tetrahydropyran-2-yl-indazol-5-yl)-2-methyl-pyrazol-3-yl]oxyethoxymethyl]-4-iodo-3-methyl-pyrazol-1-yl]acetate (700 mg, 1.06 mmol, 14% yield) as yellow oil. ¹H NMR (400 MHz, DMSO-d6) δ 7.82-7.77 (m, 2H), 7.76-7.71 (m, 1H), 5.91-5.85 (m, 1H), 5.78-5.74 (m, 1H), 5.05-5.01 (m, 2H), 4.56 (s, 2H), 4.15-4.08 (m, 1H), 4.04-4.00 (m, 1H), 3.92-3.85 (m, 1H), 3.69-3.66 (m, 3H), 3.65-3.61 (m, 2H), 3.60-3.56 (m, 2H), 3.51-3.46 (m, 3H), 2.43-2.29 (m, 2H), 2.12 (s, 3H), 2.05-1.96 (m, 2H), 1.60 (s, 2H).

Step 2. To a mixture of methyl 2-[5-[2-[4-(3-ethynyl-1-tetrahydropyran-2-yl-indazol-5-yl)-2-methyl-pyrazol-3-yl]oxyethoxymethyl]-4-iodo-3-methyl-pyrazol-1-yl]acetate (700 mg, 1.06 mmol, 1 eq) in dioxane (4 mL) was added Cu₂O (76.0 mg, 531 umol, 0.5 eq), PPh₃ (278 mg, 1.06 mmol, 1 eq) and 4,4,5,5-tetramethyl-2-(4,4,5,5-tetramethyl-1,3,2-dioxaborolan-2-yl)-1,3,2-dioxaborolane (404 mg, 1.59 mmol, 1.5 eq), the reaction mixture was stirred at 80° C. for 12 hours. On completion, the residue was diluted with water (10 mL) and extracted with EA (2×10 mL). The combined organic layers was dried over Na₂SO₄, filtered and concentrated in vacuo to give a residue. The residue was purified by flash column chromatography and then purified by reverse phase to afford 2-[4-iodo-3-methyl-5-[2-[2-methyl-4-[1-tetrahydropyran -2-yl-3-[(E)-2-(4,4,5,5-tetramethyl-1,3,2-dioxaborolan-2-yl)vinyl]indazol-5-yl]pyrazol-3-yl]oxyethoxymethyl]pyrazol-1-yl]acetate (120 mg, 152 umol, 14% yield) as white solid. LCMS: [M+H]⁺ m/z=787.0

Step 3. To a mixture of 2-|4-iodo-3-methyl-5-|2-|2-methyl-4- |1-tetrahydropyran -2-yl-3-[(E)-2-(4,4,5,5-tetramethyl-1,3,2-dioxaborolan-2-yl)vinyl]indazol-5-yl]pyrazol-3-yl]oxyethoxymethyl]pyrazol-1-yl]acetate (120 mg, 152 umol, 1 eq) in H₂O (0.2 mL) and 1,4-dioxane (1 mL) was added Pd(dppf)Cl₂ (11.1 mg, 15.2 umol, 0.1 eq) and Cs₂CO₃ (149 mg, 457 umol, 3 eq), the reaction mixture was stirred at 90° C. for 8 hours. On completion, the residue was diluted with water (5 mL) and extracted with EA (2×5 mL). The combined organic layers was dried over Na₂SO₄, filtered and concentrated in vacuo. The residue was purified by prep-HPLC to afford 2-[(17E)-5,15-dimethyl-21-tetrahydropyran-2-yl-7,10-dioxa-4,5,13,14,20,21-hexazapentacyclo[17.5.2.0^{2,6}.0^{12,16}0.0^{22,26}]hexacosa-1(25),2(6),3,12(16),14,17,19,22(26),23-nonaen-13-yl]acetic acid (32.0 mg, 30% yield) as white solid. LCMS: [M+H]⁺ m/z=519.3

Step 4. To a mixture of 2-[(17E)-5,15-dimethyl-21-tetrahydropyran-2-yl-7,10-dioxa-4,5,13,14,20,21-hexazapentacyclo[17.5.2.0^{2,6}.0^{12,16}0.0^{22,26}]hexacosa-1(25),2(6),3,12(16),14,17,19,22(26),23-nonaen-13-yl]acetic acid (27.0 mg, 52.0 umol, 1 eq) in DMF (2 mL) was added HATU (23.7 mg, 62.4 umol, 1.2 eq) and DIEA (20.1 mg, 156 umol, 27.2 uL, 3 eq), the reaction mixture was stirred at 25° C. for 0.5 hour, and then the reaction mixture was added ethanamine; hydrochloride (27.0 mg, 228 umol, 4.39 eq), the reaction mixture was stirred at 25° C. for 2 hours. On completion, the residue was diluted with water (5 mL) and extracted with EA (2×5 mL). The combined organic layers was dried over Na₂SO₄, filtered and concentrated in vacuo to afford 2-[(17E)-5,15-dimethyl-21-tetrahydropyran-2-yl-7,10-dioxa-4,5,13,14,20,21-hexazapentacyclo[17.5.2.0^{2,6}.0^{12,16}0.0^{22,26}]hexacosa-1(25),2(6),3,12(16),14,17,19,22(26),23-nonaen-13-yl]-N-ethyl-acetamide (23.0 mg, 42.1 umol, 80% yield) as colourless gum.

LCMS: [M+H]⁺ m/z=546.3

Step 5. To a mixture of 2-[(17E)-5,15-dimethyl-21-tetrahydropyran-2-yl-7,10-dioxa-4,5,13,14,20,21-hexazapentacyclo[17.5.2.0^{2,6}.0^{12,16}0.0^{22,26}]hexacosa-1(25),2(6),3,12(16),14,17,19,22(26),23-nonaen-13-yl]-N-ethyl-acetamide (21.0 mg, 38.4 umol, 1 eq) in DCM (1 mL) was added TFA (16.1 g, 141 mmol, 10.5 mL, 3684 eq), the reaction mixture was stirred at 25° C. for 1 hour. On completion, the residue was concentrated in vacuo to give a residue. The residue was purified by prep-HPLC to afford Ex. 301 (3.74 mg, 5.85 umol, 15% yield, 90% purity, TFA) as yellow solid. ¹H NMR (400 MHz, DMSO-d6) δ=13.12-12.70 (m, 1H), 8.54 (s, 1H), 8.16 (t, J=5.4 Hz, 1H), 8.10 (d, J=17.5 Hz, 1H), 7.92 (s, 1H), 7.70 (dd, J=1.3, 8.8 Hz, 1H), 7.49 (d, J=8.9 Hz, 1H), 6.94 (d, J=17.6 Hz, 1H), 4.84 (s, 2H), 4.69 (s, 2H), 4.28 (br dd, J=2.2, 4.3 Hz, 2H), 3.99 (br s, 2H), 3.74 (s, 3H), 3.14-3.08 (m, 2H), 2.39 (s, 3H), 1.05 (t, J=7.3 Hz, 3H). LCMS: [M+H]⁺ m/z=461.9

### Preparation of (5-bromo-4-iodo-2-methyl-pyrazol-3-yl)methanol (I-1-302)

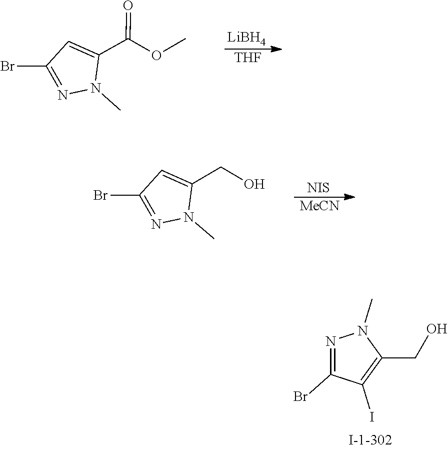

Step 1. To methyl 5-bromo-2-methyl-pyrazole-3-carboxylate (500 mg, 2.28 mmol) in THE (11.4 mL) at 0° C. was added, LiBH₄ (120 mg, 5.44 mmol). Stirred as temperature increase to RT over 18 hr. Reaction was quenched with water at 0° C. and the reaction was worked up with DCM and water (30 mL). The aqueous layer was extracted with DCM (2×20 mL). The combined organic layer was washed with brine and dried over sodium sulfate. The filtrate was concentrated and residue was crystallized with EA (10 mL) and Hexanes (30 mL) to afford (5-bromo-2-methyl-pyrazol-3-yl)methanol (333 mg, 1.74 mmol, 76.37% yield). LCMS: [M/M+2]⁺ m/z=190.9/192.9

Step 2. To (5-bromo-2-methyl-pyrazol-3-yl)methanol (333 mg, 1.74 mmol) in Acetonitrile (8.72 mL) was added NIS (470.64 mg, 2.09 mmol). Stir at 22° C. for 18 hr. Quenched with water (5 mL) and worked up with DCM and water (30 mL) and the layers were separated. The aqueous layer was extracted again with DCM (2×20 mL). The combined organic layer was washed with brine and dried over sodium sulfate. Filtrate was concentrated after filtration. Resulting residue was crystallized with EA and Hexanes to afford I-1-302 (488 mg, 1.54 mmol, 88.33% yield) as a beige solid. Further dried on house vacuum overnight. LCMS: [M/M+2]⁺ m/z=316.2/318.2

### Preparation of 2-[4-(3-ethynyl-1-tetrahydropyran-2-yl-indazol-5-yl)-2-methyl-pyrazol-3-yl]oxyethyl methanesulfonate (I-2-302)

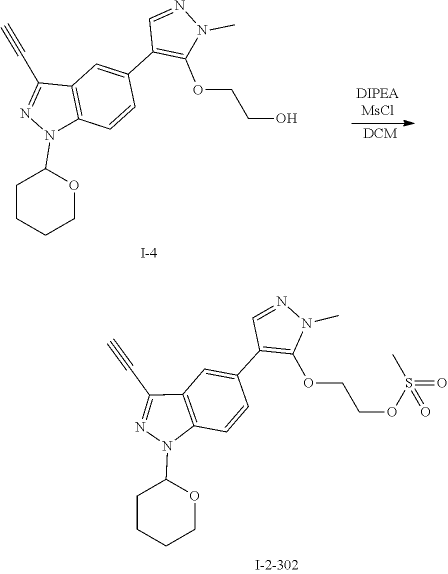

Step 1. To 2-[4-(3-ethynyl-1-tetrahydropyran-2-yl-indazol-5-yl)-2-methyl-pyrazol-3-yl]oxyethanol, I-4, (204 mg, 556.8 mol) in DCM (3 mL) was added base, DIPEA (1.67 mmol, 290 μL) followed by methanesulfonyl chloride (668.10 μmol, 52 L). Stirred at 0° C. as temperature warmed for 18 hr. Reaction was diluted with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 40-100% EA in Hexanes) provided I-2-302 (175 mg, 393.70 mol, 70.71% yield). LCMS: [M+H]⁺ m/z=444.9

### General Method C. Preparation of (17E)-8,14-dimethyl-2,8,10,11,13,14-hexahydro-3,5-etheno[1,4]dioxacyclopentadecino[10,11-c:15,14-c′:6,7-c″]tripyrazole-16-carbonitrile (Ex. 302)

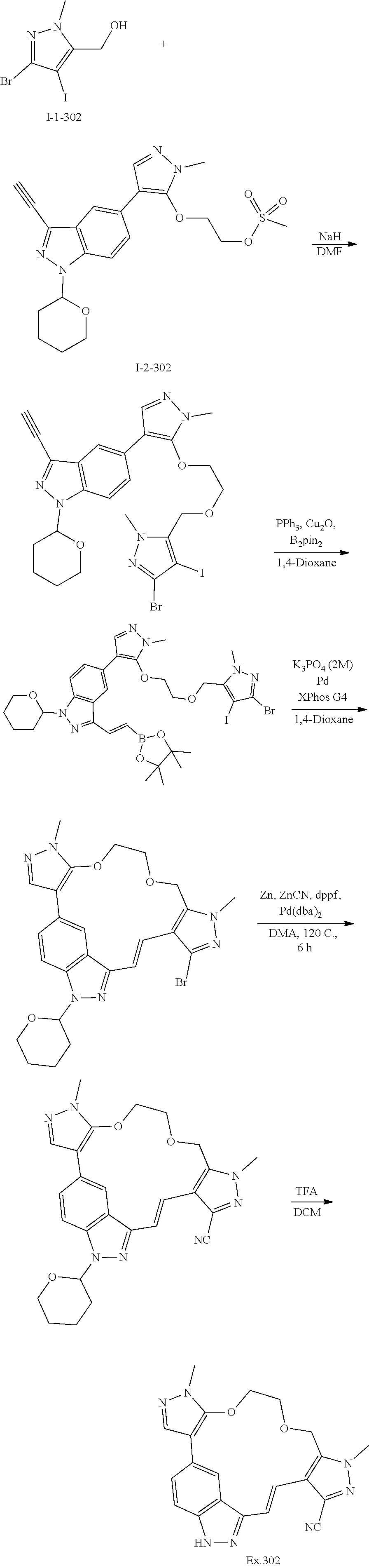

Step 1. To I-1-302 (107 mg, 337.5 μmol) in anhydrous DMF (1.1 mL) was added sodium hydride (13.50 mg, 337.5 μmol, 60% in mineral oil) at 0° C. under argon. Stirred as temperature increases to RT over 1 h. Cooled again and a solution of I-2-302 (100 mg, 224.97 mol) in DMF (1 mL) was added dropwise over 5 minutes. Stir as temperature increased to RT overnight. Cooled to 0° C. and quenched with saturated NH₄Cl (aq) (2 mL). Reaction mixture was diluted with DCM and water (10 mL) and the layers were separated. The aqueous layer was extracted again with DCM (2×10 mL). The combined organic layer was washed with brine and dried over sodium sulfate. Flash column chromatography (automated system, 12 g silica, 20-80% EA in Hexanes) twice provided I-3-302 (21.49 mg, 32.30 mol, 14.36% yield). LCMS: [M/M+2]⁺ m/z=664.8/666.8

Step 2 was performed in a similar manner to step 4 in General Method A to provided I-4-302 (19.96 mg, 25.16 mol, 77.90% yield). LCMS: [M/M+2]⁺ m/z=792.8/794.8

Step 3. To I-4-302 (66.8 mg, 81.45 mol) in solvent, 1,4-Dioxane (1.88 mL) was added K₃PO₄ (2 M, 122 μL). Stir as argon bubbled through and catalyst, Pd Xphos G4 (7.00 mg, 8.15 μmol) was added. Vessel sealed and heated to 85° C. for 1.5 hr. Reaction was diluted with DCM and water (10 mL) and the layers were separated. The aqueous layer was extracted again with DCM (2×10 mL). The combined organic layer was washed with brine and dried over sodium sulfate. Flash column chromatography (automated system, 12 g silica, 0-10% MeOH in DCM) followed by preparative HPLC (C18, 10-100% acetonitrile in water with 0.035% TFA) afforded I-5-302 (9.76 mg, 17.23 mol, 21.15% yield). LCMS: [M/M+2]⁺ m/z=538.9/540.9

Step 4. To I-5-302 (9.76 mg, 17.23 μmol) in DMA (1 mL) was added zinc (2.3 mg, 34.46 μMol) and zinc cyanide (22.3 mg, 189.52 μmol), argon was bubbled through as dppf (5.7 mg, 10.34 μmol) was added followed by Pd(dba)₂ (3 mg, 5.2 μmol). Stir under argon for about 5 minutes. Vessel was closed and heated to 120° C. and stirred for 6 hr. Reaction was diluted with DCM and water (10 mL) and the layers were separated. The aqueous layer was extracted again with DCM (2×10 mL). The combined organic layer was washed with brine and dried over sodium sulfate. Flash column chromatography twice (automated system, 12 g silica, 0-10% MeOH in DCM, then 0-10% MeOH/DCM (1/3) in EA) provided I-6-302 (3.85 mg, 7.51 mol, 43.59% yield). LCMS: [M+H]⁺ m/z=486.0

Step 5 was performed in a similar manner to step 6 in General Method A to provided Ex. 302 (1.77 mg, 4.30 mol, 68.74% yield, 97.61% purity). 1H NMR (400 MHz, DMSO-d6) δ=8.53 (s, 1H), 8.10 (d, J=17.6 Hz, 1H), 7.96 (s, 1H), 7.74 (d, J=8.9 Hz, 1H), 7.53 (d, J=8.9 Hz, 1H), 7.49 (d, J=17.4 Hz, 1H), 5.01 (s, 2H), 4.33-4.28 (m, 2H), 4.08-4.02 (m, 2H), 3.87 (s, 3H), 3.75 (s, 3H). LCMS: [M+H]⁺ m/z=401.96

### General Method D. Preparation of (17E)-8,14,16-trimethyl-2,8,10,11,13,14-hexahydro-3,5-etheno[1,4]dioxacyclopentadecino[10,11-c:15,14-c′:6,7-c″]tripyrazole (Ex. 303)

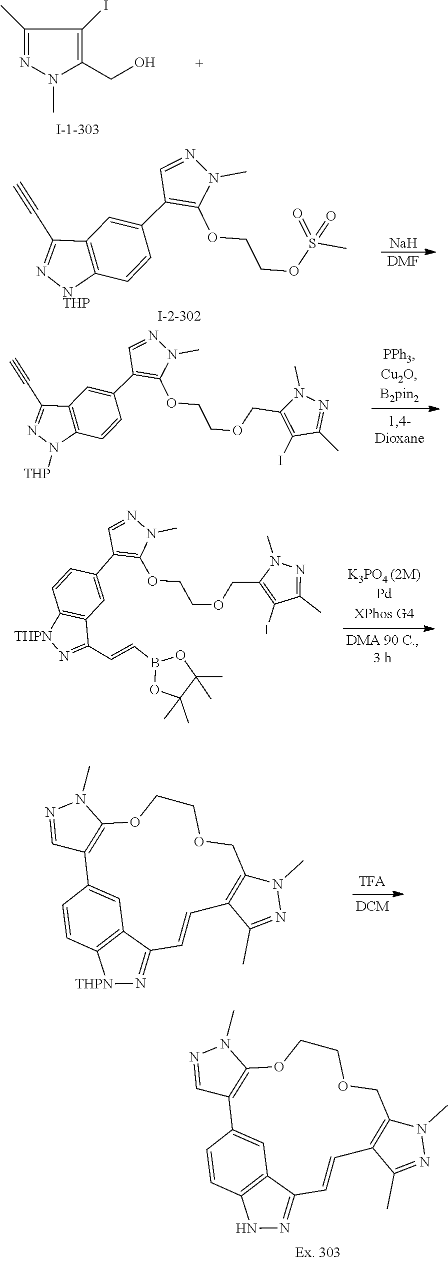

Intermediate I-1-303 was prepared similarly to I-1-302 starting from appropriate alcohol in step 2.

Step 1. To I-1-303 (56.70 mg, 224.97 mol) in DMF (1 mL) was added base, sodium hydride (8.1 mg, 202.47 μmol, 60% in mineral oil) followed by I-2-3 (50 mg, 112.49 μmol). Stirred at 22° C. for 18 hr. Reaction was cooled and quenched with 1 mL of saturated NH₄Cl(aq), diluted with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 40-100% EA in Hexanes) provided 3-ethynyl-5-[5-[2-[(4-iodo-2,5-dimethyl-pyrazol-3-yl)methoxy]ethoxy]-1-methyl-pyrazol-4-yl]-1-tetrahydropyran-2-yl-indazole (18.11 mg, 30.16 μmol, 26.81% yield). LCMS: [M+H]⁺ m/z=601.1

Step 2. To 3-ethynyl-5-[5-[2-[(4-iodo-2,5-dimethyl-pyrazol-3-yl)methoxy]ethoxy]-1-methyl-pyrazol-4-yl]-1-tetrahydropyran-2-yl-indazole (18.11 mg, 30.16 μmol) in solvent, 1,4-Dioxane (1 mL) was added 4,4,5,5-tetramethyl-2-(4,4,5,5-tetramethyl-1,3,2-dioxaborolan-2-yl)-1,3,2-dioxaborolane (11.5 mg, 45 Mol) and triphenylphosphine (11.9 mg, 45.2 mol). Stir as argon was bubbled through and catalyst, copper(I) oxide (4.3 mg, 30.2 mol) was added. Vessel sealed and heated to 80° C. for 1 hr. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 40-100% EA in Hexanes) provided 5-[5-[2-[(4-iodo-2,5-dimethyl-pyrazol-3-yl)methoxy]ethoxy]-1-methyl-pyrazol-4-yl]-1-tetrahydropyran-2-yl-3-[I(E)-2-(4,4,5,5-tetramethyl-1,3,2-dioxaborolan-2-yl)vinyl]indazole (21.97 mg, 30.16 mol, 100.00% yield). Taken forward without additional purification. LCMS: [M+H]⁺ m/z=729.2

Step 3. To 5-[5-[2-[(4-iodo-2,5-dimethyl-pyrazol-3-yl)methoxy]ethoxy]-1-methyl-pyrazol-4-yl]-1-tetrahydropyran-2-yl-3-[(E)-2-(4,4,5,5-tetramethyl-1,3,2-dioxaborolan-2-yl)vinyl]indazole (21.97 mg, 30.16 mol) in solvent, DMA (0.5 mL) was added aqueous K₃PO₄ (2 M, 45 L). Stir as argon was bubbled through and catalyst, Pd Xphos G4 (2.6 mg, 3.02 μmol) was added. Vessel sealed and heated to 90° C. for 3 hr. Reaction was diluted with DCM and water (5 ml) and the layers were separated. The aqueous layer was extracted again with DCM (2×5 mL). The combined organic layer was washed with brine and dried over sodium sulfate. Flash column chromatography (automated system, 12 g silica, 0-10% methanol in DCM) provided (14E)-16,29,30-trimethyl-31-tetrahydropyran-2-yl-32,33-dioxa-26,27,28,29,30,31-hexazapentacyclohexacosa-3,5(17),6(26),14,16(27),18,20(24),21,23(28)-nonaene (2.04 mg, 4.30 μmol, 14.25% yield).

LCMS: [M+H]+m/z=475.1

Step 4. To (14E)-16,29,30-trimethyl-31-tetrahydropyran-2-yl-32,33-dioxa-26,27,28,29,30,31-hexazapentacyclohexacosa-3,5(17),6(26),14,16(27),18,20(24),21,23(28)-nonaene (2.04 mg, 4.30 mol) in DCM (1 mL) was added trifluoroacetic acid (2.60 mmol, 0.2 mL). Stirred at 22° C. for 18 hr. Volatiles were removed under reduced pressure and residue was purified by preparative HPLC (C18, 21×100 mm, acetonitrile in water with 0.035% TFA) twice to afford Ex. 303 (1.09 mg, 2.12 μmol, 49.25% yield, 97.99% purity) as a TFA salt. 1H NMR (500 MHz, DMSO-d6) δ=12.90 (br s, 1H), 8.54 (br s, 1H), 8.06 (dd, J=2.6, 17.5 Hz, 1H), 7.92 (d, J=3.4 Hz, 1H), 7.70 (br d, J=6.9 Hz, 1H), 7.49 (dd, J=2.6, 8.3 Hz, 1H), 6.92 (dd, J=2.9, 17.8 Hz, 1H), 4.87 (d, J=2.3 Hz, 2H), 4.30 (br d, J=3.4 Hz, 2H), 4.02 (br d, J=3.4 Hz, 2H), 3.74 (d, J=2.9 Hz, 3H), 3.70 (d, J=2.9 Hz, 3H), 2.39 (d, J=2.3 Hz, 3H). LCMS: [M+H]⁺ m/z=391.0

### Preparation of tert-butyl-dimethyl-[(2S)-2-[2-methyl-4-(4,4,5,5-tetramethyl-1,3,2-dioxaborolan-2-yl)pyrazol-3-yl]oxypropoxy]silane (I-1-319)

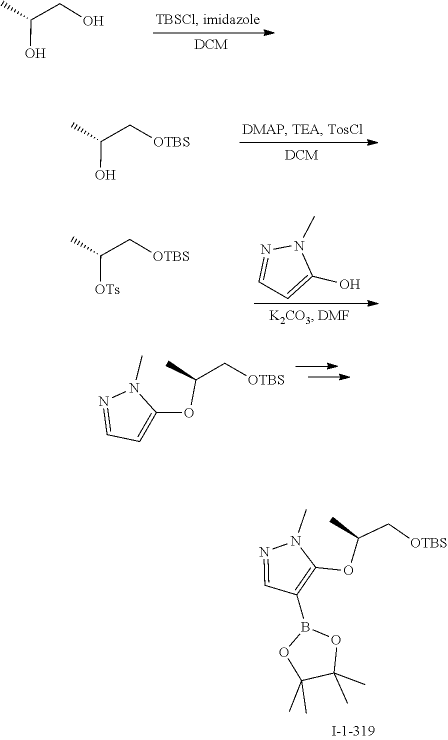

Step 1: To a solution of (2R)-propane-1,2-diol (10.0 g, 131 mmol, 1 eq) in DCM (100 mL) was added imidazole (8.95 g, 131 mmol, 1 eq) and TBSCl (19.8 g, 131 mmol, 1 eq) at 0° C. The mixture was stirred at 25° C. for 2 h. On completion, the mixture was quenched with water (200 mL) and extracted with DCM (250 mL×3), the combined organic phase was dried over anhydrous sodium sulfate, filtered and concentrated to give 5-bromo-6-chloro-3-iodo-1H-indazole (7.70 g, 99% yield) as a red solid.

Step 2. To a solution of (2R)-1-[tert-butyl(dimethyl)silyl]oxypropan-2-ol (24.0 g, 126 mmol, 1 eq) and TosCl (36.0 g, 189 mmol, 1.5 eq) in DCM (300 mL) was added DMAP (2.31 g, 18.9 mmol, 0.15 eq) and TEA (38.2 g, 378 mmol, 3 eq) at 0° C. The mixture was stirred at 25° C. for 12 h. On completion, the mixture was quenched with water (200 mL) and extracted with DCM (250 mL×3), the combined organic phase was dried over anhydrous sodium sulfate, filtered and concentrated to give [(1R)-2-[tert-butyl(dimethyl)silyl]oxy-1-methyl-ethyl]4-methylbenzenesulfonate (40.0 g, 116 mmol, 92% yield) as a yellow oil.

Step 3. To a solution of 2-methylpyrazol-3-ol (8.00 g, 81.5 mmol, 1 eq) and [(1R)-2-[tert-butyl(dimethyl)silyl]oxy-1-methyl-ethyl]4-methylbenzenesulfonate (33.7 g, 97.8 mmol, 1.2 eq) in DMF (200 mL) was added K₂CO₃ (33.8 g, 244 mmol, 3 eq). The mixture was stirred at 80° C. for 4 h. On completion, the mixture was quenched with water (400 mL) and extracted with EA (500 mL×3), the combined organic phase was dried over anhydrous sodium sulfate, filtered and concentrated to give a residue. The residue was purified by column chromatography (SiO₂, PE/EA=10:1 to 5:1) to give tert-butyl-dimethyl-[(2S)-2-(2-methylpyrazol-3-yl)oxypropoxy]silane (10.0 g, 36.9 mmol, 45% yield) as a yellow oil. LCMS: [M+H]⁺ m/z=271.7

Step 4. tert-butyl-dimethyl-[(2S)-2-(2-methylpyrazol-3-yl)oxypropoxy]silane was converted to I-1-319 in a similar manner to method to make I-2-1. LCMS: [M+H]⁺ m/z=397.2

### Preparation of 5-[5-[(1S)-2-[(5-ethyl-4-iodo-2-methyl-pyrazol-3-yl)methoxy]-1-methyl-ethoxy]-1-methyl-pyrazol-4-yl]-1-tetrahydropyran-2-yl-3-[(E)-2-(4,4,5,5-tetramethyl-1,3,2-dioxaborolan-2-yl)vinyl]indazole (I-2-319)

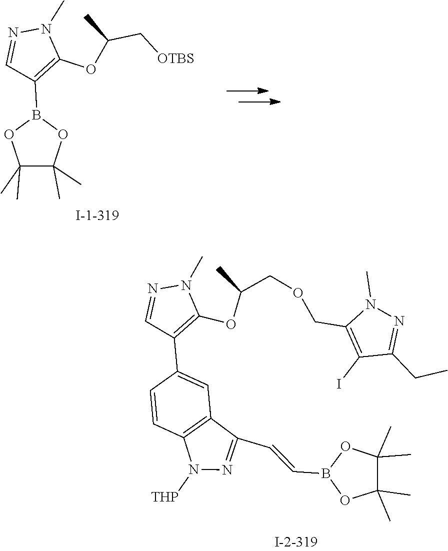

I-1-319 was converted to I-2-319 in a manner similar to those described in General Method A. LCMS: [M+H]⁺ m/z=757.2

### Preparation of (10S,17E)-16-ethyl-8,10,14-trimethyl-2,8,10,11,13,14-hexahydro-3,5-etheno[1,4]dioxacyclopentadecino[10,11-c:15,14-c′:6,7-c″]tripyrazole (Ex. 319)

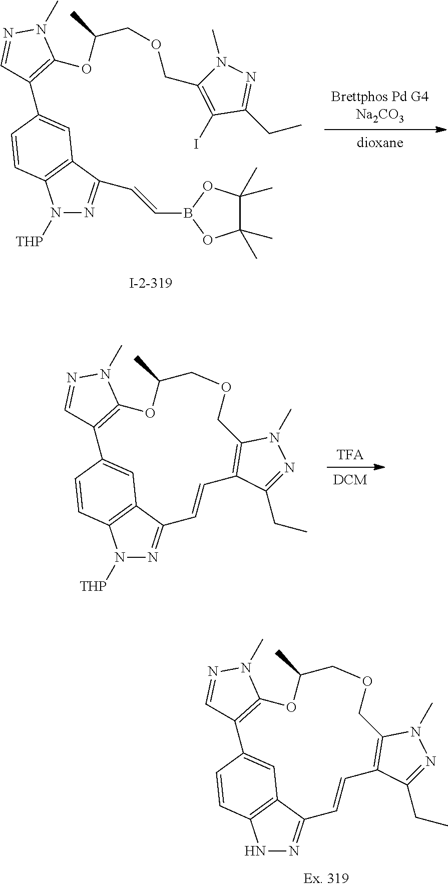

Step 1. To a solution of I-2-319 (500 mg, 660 umol, 1 eq) in dioxane (10 mL) was added Na₂CO₃ (1.5 M, 1.32 mL, 3 eq) and dicyclohexyl-[3,6-dimethoxy-2-(2,4,6-triisopropylphenyl)phenyl]phosphane;methanesulfonate;[2-[2-(methylamino)phenyl]phenyl]palladium(1+), Brettphos Pd G4 (60.8 mg, 66.1 umol, 0.1 eq). The mixture was stirred at 80° C. for 2 h under N₂. On completion, the mixture was filtered and concentrated to give a residue. The residue was purified by column chromatography (SiO₂, PE/THF=5:1 to 3:1) to give (8S,17E)-15-ethyl-5,8,13-trimethyl-21-tetrahydropyran-2-yl-7,10-dioxa-4,5,13,14,20,21-hexazapentacyclo[17.5.2.0^{2,6}.0^{12,16}.0^{22,26}]hexacosa-1(25),2(6),3,12(16),14,17,19,22(26),23-nonaene (230 mg, 320 umol, 48% yield, 70% purity) as a white solid. LCMS: [M+H]⁺ m/z=503.3

Step 2. To a solution of (8S,17E)-15-ethyl-5,8,13-trimethyl-21-tetrahydropyran-2-yl-7,10-dioxa-4,5,13,14,20,21-hexazapentacyclo[17.5.2.02,6.012,16.022,26]hexacosa-1(25),2(6), 3,12(16),14,17,19,22(26),23-nonaene (150 mg, 298 umol, 1 eq) in DCM (1 mL) was added TFA (4.62 g, 40.5 mmol, 135 eq). The mixture was stirred at 25° C. for 1 h. On completion, the mixture was concentrated to give a residue. The crude product was triturated with ACN (5 mL) at 25° C. for 5 minutes to give Example 319 (38.7 mg, 92.5 umol, 31.0% yield) as a white solid. ¹H NMR (400 MHz, METHANOL-d4) δ=8.51 (s, 1H), 8.15 (d, J=16.0 Hz, 1H), 7.79 (s, 1H), 7.64 (dd, J=1.2, 8.8 Hz, 1H), 7.46 (d, J=8.8 Hz, 1H), 6.89 (d, J=16.0 Hz, 1H), 4.78 (br d, J=16.0 Hz, 3H), 3.90-3.80 (m, 2H), 3.71 (s, 6H), 2.83 (dq, J=1.2, 8.0 Hz, 2H), 1.31 (t, J=8.0 Hz, 3H), 1.10 (d, J=6.0 Hz, 3H). [M+H]⁺ m/z=419.3

Ex. 320 was prepared in a manner similar to Method D. ¹H NMR (400 MHz, DMSO-d6) δ=8.55 (s, 1H), 7.93 (s, 1H), 7.81-7.68 (m, 2H), 7.53 (d, J=8.7 Hz, 1H), 7.11 (d, J=17.4 Hz, 1H), 4.84 (s, 2H), 4.33-4.27 (m, 2H), 4.08-4.02 (m, 2H), 3.74 (s, 3H), 2.68 (s, 3H). LCMS: [M+H]⁺ m/z=378.2

Ex. 321 was prepared in a manner similar to Method D. ¹H NMR (400 MHz, DMSO-d6) δ=8.53 (s, 1H), 8.08 (d, J=17.6 Hz, 1H), 7.95 (s, 1H), 7.74 (br d, J=8.7 Hz, 1H), 7.66-7.58 (m, 1H), 7.52 (br d, J=8.7 Hz, 1H), 7.12 (br d, J=17.6 Hz, 1H), 5.02 (s, 2H), 4.28 (br s, 2H), 4.05 (br d, J=2.5 Hz, 2H), 3.74 (s, 3H), 2.53 (br s, 3H). LCMS: [M+H]⁺ m/z=378.2

### Preparation of (4-iodo-5-methyl-isothiazol-3-yl)methanol (I-1-322)

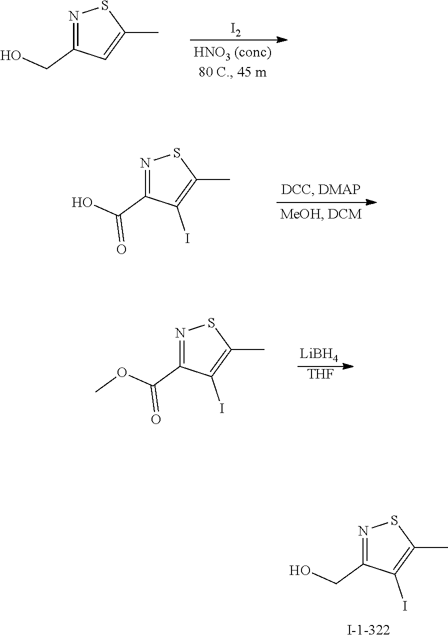

Step 1. To (5-methylisothiazol-3-yl)methanol (100 mg, 774 μmol) in a vial was added nitric acid (774 μmol, 3 mL, 70% purity) followed by Iodine (196 mg, 774 mol). The mixture was stirred and heated for 1 hr at 80° C. Reaction was cooled and carefully quenched with 1M sodium bisulfite(aq) (3 mL). The mixture was diluted with DCM and water (10 mL) and the layers were separated. The aqueous layer was extracted again with DCM (2×5 mL). The combined organic layer was washed with brine and dried over sodium sulfate. The filtrate was concentrated and triturated with hexanes to give 4-iodo-5-methyl-isothiazole-3-carboxylic acid (169 mg, 628 μmol, 81.14% yield). LCMS: [M+H]⁺ m/z=269.8

Step 2. To 4-iodo-5-methyl-isothiazole-3-carboxylic acid (167 mg, 620.7 μmol) in Methanol (1 mL) and DCM (1 mL) was added DCC (141 mg, 683 gmol), followed by DMAP (7.6 mg, 62 μmol). Stir at 22° C. for 18 hr. Reaction was diluted with DCM and water (10 mL) and the layers were separated. The aqueous layer was extracted again with DCM (2×5 mL). The combined organic layer was washed with brine and dried over sodium sulfate. Flash column chromatography (automated system with ELSD, 12 g silica, 0-60% EA in Hexanes) provided methyl 4-iodo-5-methyl-isothiazole-3-carboxylate (118.7 mg, 419.31 μmol, 67.56% yield). LCMS: [M+H]⁺ m/z=283.8

Step 3. To methyl 4-iodo-5-methyl-isothiazole-3-carboxylate (118.7 mg, 419.31 μmol) in THF (2 mL) was added lithium borohydride (18.27 mg, 838.61 μmol). Stirred as temperature increased over 1 hr. Reaction was cooled and quenched with water (5 mL) carefully and then diluted with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-60% EA in Hexanes) provided I-1-322 (82 mg, 321.47 μmol, 76.67% yield). LCMS: [M+H]⁺ m/z=255.8

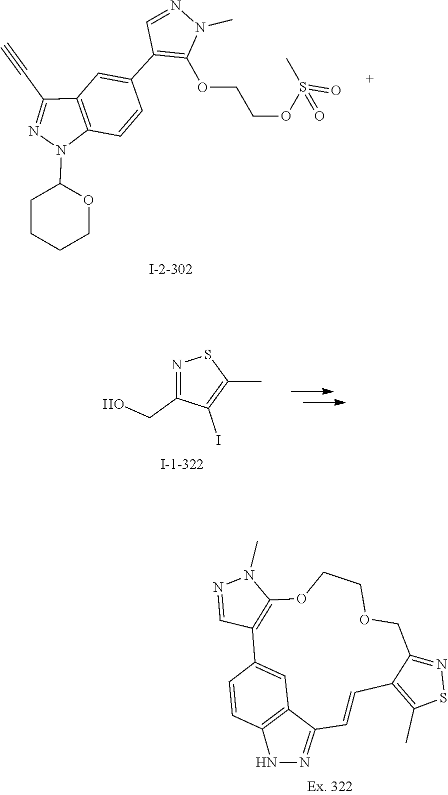

Ex. 322 was prepared in a manner similar to Method D from the appropriate intermediates: I-2-302 and I-1-322. ¹H NMR (500 MHz, DMSO-d6) δ=13.13 (s, 1H), 8.62 (s, 1H), 8.04 (d, J=17.5 Hz, 1H), 7.92 (s, 1H), 7.72 (dd, J=1.4, 8.8 Hz, 1H), 7.54 (d, J=8.8 Hz, 1H), 7.22 (d, J=17.2 Hz, 1H), 4.82 (s, 2H), 4.31 (br dd, J=2.9, 4.5 Hz, 2H), 4.07-4.00 (m, 2H), 3.74 (s, 3H), 2.77 (s, 3H). LCMS: [M+H]+n/z=393.9

| Ex # | Structure | LC | NMR (400 MHZ, DMSO-d6) |
|---|---|---|---|
| 300 | 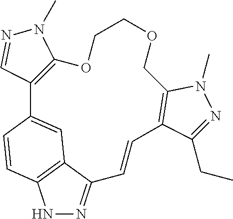 [SMILES: CCc1nn(C)c2c1/C=C/c1n[nH]c3ccc(cc13)-c1cnn(C)c1OCCOC2] | 404.8 | δ = 8.54 (s, 1H), 8.02 (d, J = 17.5 Hz, 1H), 7.92 (s, 1H), 7.70 (dd, J = 1.1, 8.8 Hz, 1H), 7.49 (d, J = 8.8 Hz, 1H), 6.90 (d, J = 17.5 Hz, 1H), 4.87 (s, 2H), 4.30 (dd, J = 2.4, 4.4 Hz, 2H), 4.05- 3.99 (m, 2H), 3.74 (s, 3H), 3.72 (s, 3H), 2.80 (q, J = 7.4 Hz, 2H), 1.27 (t, J = 7.4 Hz, 3H) |
| 301 | 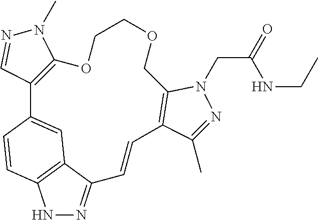 [SMILES: CCNC(=O)Cn1nc(C)c2c1COCCOc1c(cnn1C)-c1ccc3[nH]nc(c3c1)/C=C/2] | 461.9 | δ = 13.12-12.70 (m, 1H), 8.54 (s, 1H), 8.16 (t, J = 5.4 Hz, 1H), 8.10 (d, J = 17.5 Hz, 1H), 7.92 (s, 1H), 7.70 (dd, J = 1.3, 8.8 Hz, 1H), 7.49 (d, J = 8.9 Hz, 1H), 6.94 (d, J = 17.6 Hz, 1H), 4.84 (s, 2H), 4.69 (s, 2H), 4.28 (br dd, J = 2.2, 4.3 Hz, 2H), 3.99 (br s, 2H), 3.74 (s, 3H), 3.14-3.08 (m, 2H), 2.39 (s, 3H), 1.05 (t, J = 7.3 Hz, 3H) |
| 302 | 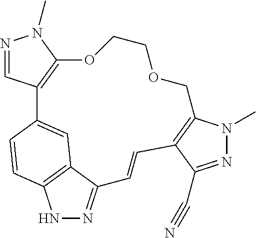 [SMILES: Cn1nc(C#N)c2c1COCCOc1c(cnn1C)-c1ccc3[nH]nc(c3c1)/C=C/2] | 402.2 | δ = 8.53 (s, 1H), 8.10 (d, J = 17.6 Hz, 1H), 7.96 (s, 1H), 7.74 (d, J = 8.9 Hz, 1H), 7.53 (d, J = 8.9 Hz, 1H), 7.49 (d, J = 17.4 Hz, 1H), 5.01 (s, 2H), 4.33- 4.28 (m, 2H), 4.08-4.02 (m, 2H), 3.87 (s, 3H), 3.75 (s, 3H |
| 303 | 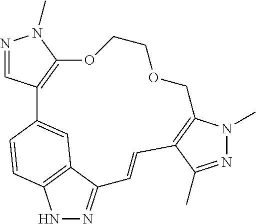 [SMILES: Cc1nn(C)c2c1/C=C/c1n[nH]c3ccc(cc13)-c1cnn(C)c1OCCOC2] | 391.0 | (500 MHz, DMSO-d6) δ = 12.90 (br s, 1H), 8.54 (br s, 1H), 8.06 (dd, J = 2.6, 17.5 Hz, 1H), 7.92 (d, J = 3.4 Hz, 1H), 7.70 (br d, J = 6.9 Hz, 1H), 7.49 (dd, J = 2.6, 8.3 Hz, 1H), 6.92 (dd, J = 2.9, 17.8 Hz, 1H), 4.87 (d, J = 2.3 Hz, 2H), 4.30 (br d, J = 3.4 Hz, 2H), 4.02 (br d, J = 3.4 Hz, 2H), 3.74 (d, J = 2.9 Hz, 3H), 3.70 (d, J = 2.9 Hz, 3H), 2.39 (d, J = 2.3 Hz, 3H) |
| 319 | 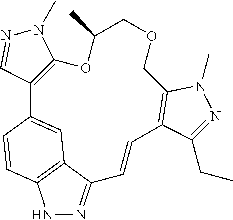 [SMILES: CCc1nn(C)c2c1/C=C/c1n[nH]c3ccc(cc13)-c1cnn(C)c1O[C@@H](C)COC2] | 419.3 | (400 MHZ, METHANOL-d4) δ = 8.51 (s, 1H), 8.15 (d, J = 16.0 Hz, 1H), 7.79 (s, 1H), 7.64 (dd, J = 1.2, 8.8 Hz, 1H), 7.46 (d, J = 8.8 Hz, 1H), 6.89 (d, J = 16.0 Hz, 1H), 4.78 (br d, J = 16.0 Hz, 3H), 3.90-3.80 (m, 2H), 3.71 (s, 6H), 2.83 (dq, J = 1.2, 8.0 Hz, 2H), 1.31 (t, J = 8.0 Hz, 3H), 1.10 (d, J = 6.0 Hz, 3H) |
| 320 | 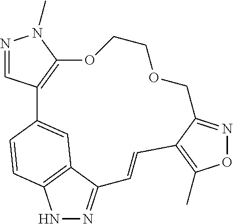 [SMILES: Cc1onc2c1/C=C/c1n[nH]c3ccc(cc13)-c1cnn(C)c1OCCOC2] | 378.2 | δ = 8.55 (s, 1H), 7.93 (s, 1H), 7.81- 7.68 (m, 2H), 7.53 (d, J = 8.7 Hz, 1H), 7.11 (d, J = 17.4 Hz, 1H), 4.84 (s, 2H), 4.33-4.27 (m, 2H), 4.08-4.02 (m, 2H), 3.74 (s, 3H), 2.68 (s, 3H |
| 321 | 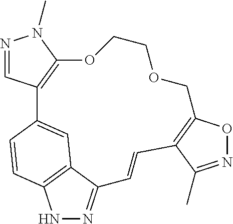 [SMILES: Cc1noc2c1/C=C/c1n[nH]c3ccc(cc13)-c1cnn(C)c1OCCOC2] | 378.3 | δ = 8.53 (s, 1H), 8.08 (d, J = 17.6 Hz, 1H), 7.95 (s, 1H), 7.74 (br d, J = 8.7 Hz, 1H), 7.66-7.58 (m, 1H), 7.52 (br d, J = 8.7 Hz, 1H), 7.12 (br d, J = 17.6 Hz, 1H), 5.02 (s, 2H), 4.28 (br s, 2H), 4.05 (br d, J = 2.5 Hz, 2H), 3.74 (s, 3H), 2.53 (br s, 3H) |
| 322 | 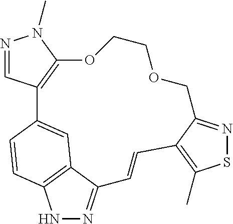 [SMILES: Cc1snc2c1/C=C/c1n[nH]c3ccc(cc13)-c1cnn(C)c1OCCOC2] | 393.9 | (500 MHZ, DMSO-d6) δ = 13.13 (s, 1H), 8.62 (s, 1H), 8.04 (d, J = 17.5 Hz, 1H), 7.92 (s, 1H), 7.72 (dd, J = 1.4, 8.8 Hz, 1H), 7.54 (d, J = 8.8 Hz, 1H), 7.22 (d, J = 17.2 Hz, 1H), 4.82 (s, 2H), 4.31 (br dd, J = 2.9, 4.5 Hz, 2H), 4.07- 4.00 (m, 2H), 3.74 (s, 3H), 2.77 (s, 3H) |

### Screen Assays

### Biochemical Assays for Inhibition of Wild-Type and Mutant EGFRs

The inhibitory activities against EGFR WT, EGFR (D770_N₇₇₁insNPG), EGFR Δ746-750 mutant, EGFR Δ746-750/C797S mutant, EGFR L858R mutant, EGFR L858R/T790M mutant, EGFR L858R/T790M/C797S mutant and ALK were evaluated at Reaction Biology Corporation (See, www.reactionbiology.com) using HotSpot assay platform, a radiometric assay based on conventional filter-binding assays, that directly measures kinase catalytic activity toward a specific substrate (Anastassiadis T, et al. Comprehensive Assay of Kinase Catalytic Activity Reveals Features of Kinase Inhibitor Selectivity. Nat Biotechnol. 2011, 29:1039-45). Briefly, specific kinase/substrate pairs along with required cofactors were prepared in reaction buffer; 20 mM Hepes pH 7.5, 10 mM MgClh, 1 mM EGTA, 0.02% Brij35, 0.02 mg/ml BSA, 0.1 mM Na₃VO₄, 2 mM DTT, 1% DMSO. Compounds were delivered into the reaction, followed -20 minutes later by addition of a mixture of ATP (Sigma, St. Louis MO) and ³³P ATP (Perkin Elmer, Waltham MA) to a final concentration of 10 μM. Reactions were carried out at room temperature for 120 min, followed by spotting of the reactions onto P81 ion exchange filter paper (Whatman Inc., Piscataway, NJ). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

TABLE 1
Inhibition of WT and mutant EGFR kinase activities.

| | | | EGFR | | | EGFR | |
|---|---|---|---|---|---|---|---|
| | EGFR | EGFR (L858R, | (L858R/T7 | EGFR (Δ746- | EGFR (Δ746- | (Δ770_N7 | EGFR |
| | (L858R) | T790M) | 90M/C797S) | 750) | 750/C797S) | 71insNPG) | wt IC₅₀, |
| Ex. # | IC₅₀, nM | IC₅₀, nM | IC₅₀, nM | IC₅₀, nM | IC₅₀, nM | IC₅₀, nM | nM |
| 1 | 848.20 | 250.60 | 258.90 | 68.37 | 176.90 | >1000 | >1000 |
| 2 | >1000 | | >1000 | >1000 | | >1000 | >1000 |
| 3 | | | 862.90 | | | | |
| 7 | 687.40 | | 201.60 | 898.20 | | 2117.00 | >1000 |
| 8 | 103.60 | | 24.34 | 44.83 | | 376.90 | 884.10 |
| 9 | 348.60 | | 27.22 | 284.20 | | 945.40 | 1167.00 |
| 10 | 181.20 | | 42.18 | 74.29 | | 712.60 | 972.60 |
| 11 | 1585.00 | 153.30 | 218.30 | 850.00 | 797.90 | >1000 | >1000 |
| 14 | 63.83 | | 19.50 | 124.10 | 34.70 | 311.70 | 339.10 |
| 15 | 104.80 | | 26.43 | 138.70 | 27.51 | 286.00 | 419.40 |
| 16 | 49.23 | | 48.35 | 131.50 | | 520.80 | 522.90 |
| 22 | 4.49 | 1.87 | 0.54 | 3.84 | 5.10 | 94.78 | 158.20 |
| 23 | 96.58 | 13.05 | 22.51 | 29.34 | 44.48 | 362.50 | 290.40 |
| 24 | >1000 | | 315.20 | 755.40 | | 1772.00 | >1000 |
| 28 | 1001.00 | | 578.20 | 819.20 | | >1000 | >1000 |
| 30 | 267.50 | | 36.82 | 216.50 | | 983.10 | 1304.00 |
| 31 | 67.69 | | 4.93 | 58.75 | | 731.50 | 927.00 |
| 32 | 242.20 | | 37.34 | 155.00 | | 456.70 | 1262.00 |
| 33 | 121.00 | 14.86 | 25.60 | 93.04 | 92.40 | 305.80 | 404.90 |
| 34 | 113.90 | | 40.90 | 153.50 | | 190.50 | 326.10 |
| 35 | 135.00 | | 30.85 | 101.20 | 99.63 | 1041.00 | 584.10 |
| 37 | 41.82 | | 17.33 | 64.04 | 49.91 | 211.70 | 156.80 |
| 39 | 221.60 | | 35.26 | 176.10 | | 1442.00 | 1073.00 |
| 43 | 144.30 | 13.46 | 34.53 | 74.81 | 133.10 | 541.30 | 543.30 |
| 44 | 285.10 | | 26.97 | 111.70 | 130.10 | >1000 | 1465.00 |
| 47 | 821.80 | 79.32 | 169.50 | 488.00 | 943.70 | >1000 | >1000 |
| 49 | 861.80 | 126.30 | 84.42 | 617.10 | 1040.00 | >1000 | >1000 |
| 55 | 35.16 | | 4.10 | 33.36 | 8.93 | 78.28 | 100.80 |
| 56 | 22.23 | 1.22 | 1.12 | 14.99 | 8.48 | 50.05 | 42.66 |
| 58 | 9.29 | | 1.57 | 11.10 | 7.29 | 221.80 | 49.17 |
| 59 | 7.63 | 0.70 | 1.02 | 18.63 | 2.42 | 126.80 | 44.11 |
| 60 | 100.70 | 6.86 | 3.27 | 89.69 | 89.43 | 677.90 | 580.60 |
| 61 | 1.23 | 0.29 | 0.17 | 1.08 | 1.96 | 9.21 | 6.19 |
| 63 | 827.80 | 43.34 | 33.49 | 295.70 | 227.60 | >1000 | 781.70 |
| 69 | 121.00 | 14.86 | 25.60 | 93.04 | 92.40 | 305.80 | 404.90 |
| 70 | 70.09 | 3.81 | 3.68 | 32.29 | 27.11 | 73.59 | 98.74 |
| 74 | 36.13 | 12.47 | 15.83 | 74.10 | 39.78 | 108.80 | 85.97 |
| 75 | 55.93 | 17.54 | 21.22 | 59.70 | 28.53 | 84.27 | 295.10 |
| 76 | 155.70 | 42.93 | 63.42 | 147.00 | 90.75 | 232.90 | 538.30 |
| 77 | 2084.00 | 427.00 | 453.40 | 749.40 | 803.30 | 1882.00 | >1000 |
| 78 | 688.40 | 205.60 | 295.80 | 211.60 | 774.60 | 909.90 | >1000 |
| 79 | 526.10 | 57.06 | 67.67 | 596.20 | 284.40 | 951.00 | 1908.00 |
| 80 | 24.72 | 4.37 | 11.22 | 38.72 | 39.20 | 59.11 | 50.81 |
| 81 | 16.76 | 1.70 | 7.42 | 29.63 | 33.37 | 40.28 | 43.76 |
| 82 | 15.26 | 1.24 | 5.77 | 27.87 | 29.84 | 56.75 | 67.83 |
| 83 | 19.04 | 1.48 | 5.29 | 34.62 | 31.28 | 55.41 | 61.47 |
| 84 | 284.80 | 30.78 | 28.23 | 380.60 | 130.00 | 551.30 | 1076.00 |
| 85 | >1000 | >1000 | >1000 | >1000 | >1000 | >1000 | >1000 |
| 86 | >1000 | >1000 | >1000 | >1000 | >1000 | >1000 | >1000 |
| 87 | >1000 | >1000 | >1000 | >1000 | >1000 | >1000 | >1000 |
| 89 | 4.66 | 0.06 | 0.45 | 19.71 | 6.67 | 191.00 | 63.97 |
| 90 | 1.02 | 0.06 | 0.81 | 11.61 | 4.65 | 81.82 | 17.06 |
| 91 | >1000 | 400.40 | >1000 | >1000 | >1000 | >1000 | >1000 |
| 92 | 4.10 | 0.41 | 3.26 | 7.95 | 6.65 | 6.34 | 14.49 |
| 93 | 11.45 | 0.86 | 5.02 | 49.16 | 16.68 | 138.10 | 69.15 |
| 94 | 9.99 | 0.82 | 5.75 | 17.65 | 14.09 | 17.23 | 44.12 |
| 95 | 30.13 | 1.95 | 4.93 | 121.00 | 23.03 | 524.30 | 167.10 |
| 96 | 74.89 | 5.29 | 9.45 | 284.70 | 94.14 | 195.80 | 242.50 |
| 97 | 131.80 | 10.80 | 28.47 | 221.00 | 100.80 | 304.10 | 412.60 |
| 98 | 41.06 | 3.56 | 12.12 | 84.93 | 26.96 | 148.10 | 119.10 |
| 100 | 87.71 | 10.40 | 19.21 | 197.90 | 91.04 | 250.30 | 289.50 |
| 102 | 25.41 | 4.10 | 13.05 | 48.12 | 44.74 | 94.54 | 62.40 |
| 103 | 103.10 | 11.48 | 40.43 | 104.80 | 170.80 | 343.50 | 358.00 |
| 106 | 34.81 | 2.49 | 10.83 | 57.63 | 68.51 | 111.50 | 107.20 |
| 107 | 13.63 | 2.28 | 11.33 | 34.14 | 42.18 | 66.47 | 41.90 |
| 108 | 26.18 | 3.86 | 11.92 | 30.85 | 49.20 | 103.00 | 101.40 |
| 109 | 164.90 | 15.93 | 47.10 | 38.01 | 152.10 | 298.10 | 855.60 |
| 110 | 30.44 | 4.99 | 8.54 | 14.41 | 28.51 | 112.20 | 150.40 |
| 111 | 8.04 | 0.39 | 1.91 | 3.29 | 9.18 | 16.84 | 63.29 |
| 112 | 1.31 | 0.21 | 0.98 | 0.61 | 2.75 | 1.12 | 8.28 |
| 113 | 96.80 | 8.66 | 9.91 | 69.87 | 67.41 | 727.50 | 488.50 |
| 114 | 0.10 | 0.11 | 0.98 | 0.17 | 0.92 | 0.91 | 0.38 |
| 115 | 37.28 | 1.60 | 14.02 | 12.94 | 60.07 | 32.30 | 147.00 |
| 116 | 0.58 | 0.38 | 2.71 | 1.06 | 2.85 | 18.47 | 3.98 |
| 117 | 19.99 | 2.20 | 6.18 | 6.91 | 8.36 | 53.45 | 70.12 |
| 118 | 34.38 | 4.65 | 8.42 | 21.10 | 31.66 | 65.70 | 130.40 |
| 119 | 0.61 | 0.26 | 2.12 | 0.63 | 1.25 | 8.50 | 2.34 |
| 120 | 7.91 | 0.33 | 2.62 | 7.76 | 5.80 | 99.75 | 41.30 |
| 121 | 94.13 | 6.14 | 13.00 | 40.68 | 41.33 | 283.10 | 295.50 |
| 122 | 11.95 | 1.46 | 2.09 | 7.45 | 6.79 | 58.02 | 94.18 |
| 123 | 1.50 | 0.33 | 0.75 | 1.30 | 0.80 | 10.10 | 11.44 |
| 124 | 18.29 | 3.32 | 2.32 | 18.34 | 13.88 | 139.90 | 168.30 |
| 125 | 11.91 | 43.58 | 7.49 | 20.58 | 4.99 | 175.10 | 137.70 |
| 126 | 18.64 | 2.07 | 2.80 | 16.09 | 19.84 | 132.80 | 164.40 |
| 127 | 1.81 | 0.15 | 0.43 | 2.82 | 0.85 | 25.56 | 11.11 |
| 128 | 21.89 | 0.77 | 1.01 | 33.74 | 15.22 | 382.90 | 263.80 |
| 129 | 0.47 | 0.09 | 0.33 | 0.58 | 0.40 | 6.50 | 3.02 |
| 130 | 84.34 | 7.32 | 5.66 | 81.06 | 38.40 | 850.60 | 456.80 |
| 131 | 4.02 | 0.57 | 1.90 | 1.65 | 4.09 | 6.79 | 17.67 |
| 132 | 157.00 | 20.08 | 35.32 | 39.37 | 67.03 | 283.60 | 720.60 |
| 133 | 0.09 | 0.07 | 0.24 | 0.12 | 0.35 | 1.62 | 0.54 |
| 134 | 182.40 | 24.01 | 12.64 | 173.30 | 90.75 | >1000 | 1210.00 |
| 135 | 128.10 | 19.96 | 38.61 | 48.27 | 97.70 | 802.40 | 828.60 |
| 136 | 14.56 | 0.80 | 1.54 | 15.19 | 12.12 | 334.20 | 103.00 |
| 137 | 7.80 | 0.60 | 1.02 | 6.59 | 4.80 | 129.10 | 55.30 |
| 138 | 3.50 | 0.61 | 0.55 | 3.64 | 1.40 | 85.17 | 23.76 |
| 140 | 11.55 | 1.19 | 1.30 | 9.35 | 6.14 | 185.60 | 84.82 |
| 142 | 0.90 | 1.29 | 1.83 | 1.11 | 1.32 | 16.69 | 2.14 |
| 143 | 39.61 | 1.58 | 4.63 | 39.44 | 41.25 | 1626.00 | 320.50 |
| 144 | 1.47 | 0.41 | 0.70 | 1.88 | 1.93 | 24.47 | 8.81 |
| 147 | 0.44 | 0.11 | 0.19 | 0.59 | 0.39 | 9.08 | 3.16 |
| 148 | 42.36 | 4.25 | 2.77 | 35.05 | 23.65 | 402.90 | 372.10 |
| 150 | 2.31 | 0.45 | 0.37 | 3.21 | 2.58 | 33.43 | 6.36 |
| 151 | 0.22 | 0.48 | 0.46 | 0.30 | 0.72 | 2.55 | 0.87 |
| 152 | 20.66 | 0.58 | 0.62 | 61.53 | 86.39 | 94.50 | 112.00 |
| 155 | 0.34 | 0.45 | 0.62 | 0.62 | 0.86 | 6.36 | 1.74 |
| 156 | 4.34 | 0.88 | 1.04 | 1.77 | 3.62 | 64.89 | 19.26 |
| 159 | 34.67 | 1.54 | 0.74 | 26.96 | 14.06 | 116.10 | 160.50 |
| 160 | 34.40 | 1.42 | 1.08 | 13.59 | 19.46 | 245.80 | 62.97 |
| 161 | 47.11 | 3.10 | 1.40 | 20.00 | 13.45 | 190.50 | 170.80 |
| 162 | 47.37 | 1.40 | 0.90 | 30.55 | 33.55 | 523.40 | 237.60 |
| 163 | 8.20 | 0.85 | 0.86 | 7.33 | 4.72 | 51.96 | 29.56 |
| 164 | 16.67 | 3.23 | 1.76 | 33.40 | 20.17 | 95.26 | 219.70 |
| 165 | 6.08 | 0.47 | 0.44 | 4.84 | 2.33 | 19.02 | 28.54 |
| 166 | 12.78 | 0.72 | 0.55 | 15.00 | 8.10 | 45.59 | 99.82 |
| 167 | 35.69 | 3.42 | 8.89 | 23.83 | 13.62 | 47.09 | 123.30 |
| 168 | 1.88 | 0.72 | 2.72 | 5.96 | 1.38 | 13.89 | 16.61 |
| 169 | 2.01 | 0.48 | 1.26 | 3.24 | 1.60 | 36.06 | 19.28 |
| 170 | 309.90 | 36.59 | 25.92 | 115.90 | 30.21 | 328.70 | 907.50 |
| 171 | 7.50 | 0.50 | 1.32 | 38.30 | 8.74 | 86.01 | 67.17 |
| 172 | 0.60 | 0.48 | 1.34 | 2.10 | 0.69 | 6.61 | 1.84 |
| 173 | 512.60 | 45.50 | 27.41 | 532.90 | 127.00 | >1000 | >1000 |
| 174 | 127.10 | 10.87 | 4.88 | 133.00 | 27.19 | 996.20 | 752.40 |
| 175 | 42.59 | 2.06 | 3.39 | 67.82 | 8.97 | 713.20 | 175.50 |
| 176 | 55.47 | 6.94 | 13.13 | 24.86 | 35.17 | 83.42 | 152.20 |
| 177 | 0.38 | 0.60 | 1.78 | 0.56 | 0.75 | 1.56 | 0.67 |
| 178 | 8.73 | 4.35 | 19.33 | 6.90 | 5.25 | 231.60 | 22.20 |
| 179 | >1000 | 1014.00 | 507.00 | >1000 | >1000 | >1000 | >1000 |
| 181 | 1.80 | 1.29 | 2.50 | 2.23 | 1.68 | 84.37 | 12.50 |
| 182 | 24.74 | 0.54 | 1.37 | 23.96 | 6.60 | 299.90 | 139.00 |
| 183 | 61.48 | 0.65 | 1.05 | 53.81 | 17.74 | 533.89 | 194.71 |
| 186 | 14.60 | 1.48 | 0.46 | 12.87 | 3.06 | 59.07 | 43.84 |
| 187 | 6.88 | 0.47 | 0.33 | 8.88 | 7.84 | 74.65 | 25.88 |
| 191 | 5.61 | 4.23 | 2.63 | 9.04 | 4.19 | 102.00 | 19.80 |
| 193 | 36.59 | 2.42 | 1.05 | 10.21 | 5.92 | 77.34 | 90.07 |
| 195 | 39.89 | 6.18 | 3.67 | 18.61 | 8.68 | 156.20 | 99.74 |
| 197 | 95.23 | 4.58 | 2.18 | 62.01 | 29.75 | 217.20 | 194.30 |
| 198 | 13.11 | 0.89 | 0.26 | 5.84 | 1.96 | 47.05 | 36.79 |
| 199 | 87.79 | 64.39 | 43.94 | 146.80 | 42.51 | >1000 | 1284.00 |
| 200 | 2.01 | 1.41 | 0.69 | 4.08 | 1.93 | 59.87 | 6.46 |
| 202 | 98.75 | 3.38 | 2.00 | 45.04 | 20.72 | 423.40 | 179.20 |
| 203 | 0.12 | 0.17 | 0.78 | 0.11 | 0.23 | 0.65 | 0.23 |
| 204 | 5.50 | 1.77 | 2.53 | 1.85 | 1.93 | 20.07 | 5.49 |
| 205 | 16.34 | 1.00 | 1.13 | 5.17 | 1.42 | 136.90 | 58.29 |
| 206 | 1.69 | 0.52 | 1.46 | 0.76 | 0.74 | 43.54 | 3.33 |
| 207 | >1000 | >1000 | >1000 | >1000 | >1000 | >1000 | >1000 |
| 211 | 400.00 | >1000 | >1000 | >1000 | 2872.00 | >1000 | >1000 |
| 212 | 52.34 | 0.57 | 0.42 | 16.91 | 3.87 | 138.80 | 153.70 |
| 213 | 82.57 | 0.42 | 0.20 | 21.06 | 5.07 | 431.00 | 295.90 |
| 214 | 0.35 | 0.20 | 0.17 | 0.22 | 0.48 | 8.31 | 0.77 |
| 215 | 1.70 | 1.49 | 0.89 | 0.69 | 1.93 | 3.75 | 1.71 |
| 216 | 2.22 | 0.44 | 0.37 | 0.47 | 0.63 | 28.91 | 2.43 |
| 217 | 37.06 | 0.69 | 1.42 | 3.61 | 1.94 | 107.10 | 71.68 |
| 218 | 28.76 | 1.99 | 2.04 | 8.48 | 16.64 | 161.10 | 118.90 |
| 219 | 4.73 | 5.30 | 3.84 | 3.02 | 5.13 | 21.23 | 7.25 |
| 220 | 515.10 | 23.09 | 27.49 | 398.10 | 474.80 | 574.50 | 1441.00 |
| 221 | 0.97 | 0.21 | 0.14 | 0.55 | 0.35 | 10.35 | 4.14 |
| 222 | 4.75 | 0.31 | 0.18 | 1.77 | 2.17 | 57.78 | 27.76 |
| 223 | 7.07 | 0.61 | 0.34 | 4.07 | 3.12 | 102.20 | 42.59 |
| 225 | 12.20 | 0.57 | 0.21 | 5.39 | 1.44 | 65.44 | 51.81 |
| 226 | 50.74 | 1.19 | 0.60 | 8.98 | 6.43 | 222.30 | 120.60 |
| 227 | 60.13 | 0.84 | 0.36 | 9.86 | 6.82 | 166.80 | 251.10 |
| 228 | 25.24 | 25.87 | 10.57 | 12.21 | 44.39 | 481.50 | 52.72 |
| 229 | 7.10 | 4.07 | 1.24 | 1.94 | 4.05 | 38.61 | 24.80 |
| 232 | 0.22 | 0.13 | 0.11 | 0.08 | 0.55 | 1.39 | 0.41 |
| 233 | 41.48 | 1.32 | 1.61 | 10.68 | 25.00 | 256.20 | 150.90 |
| 236 | 11.97 | 0.80 | 1.06 | 5.11 | 11.52 | 68.91 | 26.84 |
| 238 | 73.01 | 10.22 | 11.72 | 17.51 | 41.29 | 219.50 | 203.20 |
| 239 | 30.52 | 36.35 | 8.44 | 18.30 | 9.44 | 527.50 | 62.08 |
| 240 | 775.40 | 5.92 | 10.53 | 825.60 | 652.90 | >1000 | >1000 |
| 241 | 0.30 | 0.30 | 0.28 | 0.13 | 0.31 | 0.18 | 0.40 |
| 241 | 0.27 | 0.19 | 0.18 | 0.15 | 0.23 | 0.73 | 0.52 |
| 242 | 124.10 | 9.27 | 4.93 | 55.52 | 40.31 | 303.90 | 380.70 |
| 246 | 5.14 | 1.71 | 4.42 | 8.71 | 3.18 | 39.52 | 23.31 |
| 252 | 2.38 | 1.91 | 5.35 | 1.31 | 2.29 | 15.66 | 4.74 |
| 253 | 1.66 | 2.55 | 5.27 | 0.75 | 1.98 | 3.21 | 1.63 |
| 255 | 1.18 | 3.01 | 6.62 | 0.95 | 1.98 | 4.05 | 1.43 |
| 258 | 1.56 | 0.48 | 2.17 | 1.22 | 1.26 | 15.76 | 5.72 |
| 259 | 66.50 | 9.39 | 21.80 | 40.05 | 22.87 | 499.50 | 329.30 |
| 260 | 4.55 | 0.90 | 3.19 | 3.50 | 3.74 | 25.31 | 13.63 |
| 261 | 21.55 | 1.92 | 5.10 | 23.50 | 5.93 | 230.40 | 93.69 |
| 262 | 40.06 | 4.98 | 1.67 | 8.48 | 7.12 | 98.35 | 94.55 |
| 263 | 3.97 | 0.75 | 0.50 | 1.28 | 1.79 | 18.24 | 6.94 |
| 264 | 491.30 | 18.00 | 8.63 | 162.40 | 70.01 | 826.50 | 814.60 |
| 266 | >1000 | 282.50 | 60.43 | 971.30 | 577.30 | >1000 | >1000 |
| 267 | 240.60 | 8.31 | 5.42 | 116.90 | 106.90 | 1234.00 | 769.40 |
| 268 | 4.35 | 1.04 | 0.52 | 2.01 | 1.32 | 30.53 | 7.91 |
| 269 | 8.53 | 1.82 | 0.67 | 5.25 | 2.56 | 102.30 | 20.33 |
| 270 | 161.10 | 4.58 | 1.10 | 93.56 | 20.49 | 634.50 | 543.00 |
| 271 | 1580.00 | 56.74 | 31.62 | 471.60 | 421.90 | >1000 | >1000 |
| 319 | 181.9 | 24.72 | 60.47 | 217.9 | 425.1 | 1489 | 765.9 |

TABLE 2
Inhibition of ALK kinase activity

| Ex. # | ALK IC₅₀ (nM) |
|---|---|
| 8 | 2.09 |
| 22 | 1.35 |
| 35 | 6.93 |
| 58 | 4.79 |
| 119 | 1.05 |
| 120 | 1.82 |
| 134 | 21.6 |
| 147 | 0.99 |
| 159 | 3.72 |
| 163 | 1.13 |
| 181 | 1.05 |
| 200 | 1.49 |
| 214 | 2.47 |
| 215 | 2.58 |
| 216 | 2.04 |
| 219 | 1.31 |
| 232 | 0.90 |
| 256 | 2.00 |
| 275 | 1.46 |
| 277 | 9.87 |
| 280 | 1.08 |
| 288 | 1.82 |
| 289 | 15.4 |
| 300 | 5.79 |
| 301 | 29.01 |
| 302 | 19.95 |
| 303 | 27.52 |
| 319 | 5.054 |
| 320 | 32.22 |
| 321 | 20.84 |
| 322 | 59.9 |

### Inhibition of CLK, CLK2, CLK3 and CLK4 Kinase Activity Evaluated at Nanosyn

Kinase protein and substrate were pre-diluted in the HEPES assay buffer (100 mM HEPES, pH 7.5, 0.01% Triton X-100, 0.1% BSA, 5 mM MgCl₂, 1 mi DTT, 10 Sodium Orthovanadate, 10 M Beta-Glycerophosphate) dispensed into 384 well plate (5 μL per well). Control samples (0%-inhibition in the absence of inhibitor, DMSO only) and 100%-inhibition (in the absence of enzyme) were assembled in replicates of six and were used to calculate %-inhibition in the presence of compounds. Test compounds were added to the protein samples by acoustic dispensing (Labcyte Echo550). Concentration of DMSO was equalized to 1% in all samples. Reactions were initiated by addition of ATP by acoustic dispensing (Labcyte Echo550) and incubated according to assay specific incubation time. After incubation. 5 μL of Promega ADP-Glo reagent was added and incubated for 40 minutes. After 40 minutes, 10 uL of Promega kinase detection reagent was added. After 10 min of incubation with Kinase detection reagent, the luminescence was read on microplate reader (Biotek Synergy).

TABLE 3
Inhibition of CLK1, CLK2, CLK3 and CLK4 kinase activity

| | CLK1 | CLK2 | CLK3 | CLK4 |
|---|---|---|---|---|
| Ex. # | IC₅₀, nM | IC₅₀, nM | IC₅₀, nM | IC₅₀, nM |
| 6 | 0.34 | 0.23 | 178.00 | 0.52 |
| 7 | 0.20 | 0.08 | 45.20 | 0.48 |
| 43 | 0.10 | 0.14 | 236.00 | 0.25 |
| 47 | 9.92 | 48.30 | 1000.00 | 2.70 |
| 52 | 0.24 | 0.19 | 148.00 | 0.56 |
| 55 | 0.09 | 0.13 | 185.00 | 0.58 |
| 58 | 0.20 | 0.38 | 160.00 | 0.53 |
| 59 | 0.82 | 2.13 | 541.00 | 2.03 |
| 61 | 0.21 | 0.06 | 21.40 | 0.76 |
| 109 | 0.46 | 0.09 | 2.00 | 0.85 |
| 112 | 0.17 | 0.04 | 0.19 | 0.50 |
| 114 | 0.23 | 0.12 | 20.90 | 0.46 |
| 116 | 0.11 | 0.04 | 10.90 | 0.40 |
| 119 | 0.14 | 0.04 | 19.40 | 0.33 |
| 120 | 0.12 | 0.08 | 112.00 | 0.27 |
| 122 | 0.27 | 0.15 | 21.20 | 0.60 |
| 123 | 0.17 | 0.10 | 171.00 | 0.45 |
| 124 | 0.15 | 0.29 | 186.00 | 0.37 |
| 125 | 3.44 | 2.81 | 1000.00 | 4.31 |
| 126 | 0.32 | 0.12 | 188.00 | 0.49 |
| 132 | 0.19 | 0.02 | 0.28 | 0.30 |
| 134 | 0.29 | 0.19 | 230.00 | 0.61 |
| 137 | 1.37 | 2.13 | 79.20 | 1.55 |
| 141 | 0.11 | 0.03 | 41.90 | 0.13 |
| 142 | 11.90 | 13.90 | 1000.00 | 5.91 |
| 143 | 0.19 | 0.02 | 12.10 | 0.61 |
| 144 | 0.21 | 0.05 | 168.00 | 0.55 |
| 145 | 0.21 | 0.04 | 99.60 | 0.52 |
| 147 | 0.13 | 0.02 | 5.21 | 0.32 |
| 151 | 0.24 | 0.04 | 23.00 | 0.52 |
| 155 | 0.43 | 0.14 | 87.40 | 0.81 |
| 156 | 0.42 | 0.13 | 63.60 | 0.40 |
| 158 | 0.14 | 0.40 | 664.00 | 0.31 |
| 159 | 0.14 | 0.07 | 1000.00 | 0.38 |
| 160 | 0.22 | 0.30 | 38.20 | 0.41 |
| 161 | 0.27 | 0.14 | 164.00 | 0.57 |
| 162 | 0.11 | 0.07 | 784.00 | 0.25 |
| 163 | 0.28 | 0.06 | 215.00 | 0.63 |
| 164 | 0.39 | 0.75 | 1000.00 | 0.59 |
| 165 | 0.10 | 0.02 | 72.40 | 0.19 |
| 167 | 0.38 | 0.23 | 108.00 | 0.55 |
| 168 | 0.39 | 0.33 | 238.00 | 0.77 |
| 169 | 0.47 | 0.49 | 346.00 | 1.13 |
| 170 | 0.38 | 0.36 | 1000.00 | 0.43 |
| 171 | 0.85 | 0.62 | 977.00 | 0.57 |
| 172 | 0.32 | 0.08 | 64.70 | 0.59 |
| 173 | 0.70 | 0.89 | 1000.00 | 1.26 |
| 174 | 0.16 | 0.21 | 128.00 | 0.51 |
| 176 | 1.54 | 11.80 | 1000.00 | 1.44 |
| 177 | 0.47 | 0.23 | 59.10 | 1.08 |
| 178 | 1.08 | 2.38 | 61.80 | 1.56 |
| 179 | 2.75 | 9.57 | 1000.00 | 1.66 |
| 181 | 0.87 | 0.68 | 56.20 | 2.60 |
| 183 | 0.82 | 0.24 | 155.00 | 1.83 |
| 186 | 0.24 | 0.18 | 126.00 | 1.00 |
| 187 | 0.48 | 0.30 | 311.00 | 1.35 |
| 191 | 2.42 | 4.37 | 43.40 | 2.41 |
| 193 | 31.80 | 45.40 | 1000.00 | 21.00 |
| 195 | 36.60 | 53.50 | 435.00 | 22.30 |
| 197 | 1.16 | 3.06 | 78.60 | 1.59 |
| 198 | 0.76 | 1.61 | 1000.00 | 1.63 |
| 199 | 1.05 | 2.29 | 19.30 | 1.79 |
| 200 | 0.48 | 1.00 | 194.00 | 1.53 |
| 203 | 0.72 | 0.14 | 151.00 | 1.59 |
| 206 | 1.67 | 1.72 | 477.00 | 3.69 |
| 208 | 0.59 | 0.13 | 220.00 | 1.53 |
| 211 | 270.00 | 114.00 | 1.00 | 397.00 |
| 212 | 5.27 | 7.14 | 237.00 | 5.31 |
| 213 | 5.75 | 6.11 | 1.00 | 5.07 |
| 214 | 2.37 | 2.51 | 592.00 | 7.25 |
| 215 | 3.33 | 2.10 | 964.00 | 9.08 |
| 216 | 2.02 | 1.28 | 115.00 | 5.39 |
| 219 | 2.05 | 4.51 | 146.00 | 5.62 |
| 220 | 2.76 | 8.84 | 1.00 | 4.31 |
| 222 | 2.31 | 3.17 | 306.00 | 2.46 |
| 223 | 0.85 | 0.28 | 30.00 | 2.11 |
| 224 | 0.69 | 1.16 | 253.00 | 1.46 |
| 225 | 1.11 | 0.49 | 77.70 | 1.92 |
| 226 | 0.87 | 0.99 | 369.00 | 1.44 |
| 227 | 4.90 | 2.59 | 471.00 | 4.63 |
| 228 | 21.90 | 8.60 | 1000.00 | 34.00 |
| 229 | 131.00 | 59.60 | 1000.00 | 68.50 |
| 232 | 0.77 | 0.45 | 42.00 | 1.49 |
| 233 | 0.71 | 0.43 | 403.00 | 0.89 |
| 236 | 0.66 | 1.73 | 397.00 | 1.03 |
| 238 | 0.90 | 2.44 | 1000.00 | 1.07 |
| 240 | 1.12 | 12.50 | 1000.00 | 1.52 |
| 246 | 0.75 | 1.36 | 386.00 | 1.33 |
| 252 | 0.83 | 0.42 | 168.00 | 1.59 |
| 255 | 0.82 | 0.27 | 9.89 | 1.60 |
| 256 | 1.38 | 1.35 | 94.50 | 4.05 |
| 258 | 0.50 | 0.23 | 17.20 | 1.04 |
| 259 | 0.90 | 4.29 | 1000.00 | 1.30 |
| 260 | 4.90 | 5.21 | 361.00 | 3.15 |
| 261 | 0.20 | 0.33 | 209.00 | 0.44 |
| 262 | 0.82 | 0.49 | 104.00 | 1.50 |
| 263 | 0.64 | 0.71 | 32.70 | 1.31 |
| 264 | 1.06 | 3.08 | 519.00 | 1.59 |
| 268 | 0.57 | 0.18 | 45.10 | 1.22 |
| 269 | 0.94 | 0.32 | 238.00 | 1.76 |
| 275 | 0.80 | 1.03 | 241.00 | 2.25 |
| 277 | 4.28 | 2.99 | 51.10 | 2.77 |
| 280 | 0.52 | 0.24 | 242.00 | 1.96 |
| 288 | 0.32 | 0.24 | 131.00 | 1.31 |
| 290 | 69.40 | 102.00 | 1000.00 | 38.10 |
| 292 | 1.01 | 4.87 | 1000.00 | 1.36 |
| 294 | 1.27 | 3.25 | 1000.00 | 1.37 |
| 295 | 0.64 | 0.78 | 13.80 | 1.12 |
| 296 | 1.20 | 1.15 | 33.40 | 1.57 |
| 300 | 0.332 | 36.5 | 0.862 | 0.225 |
| 302 | 1.19 | 1000 | 1.23 | 1.33 |
| 320 | 0.324 | 69.7 | 0.2 | 0.155 |
| 321 | 1.35 | 418 | 1.92 | 0.847 |
| 322 | 1.04 | 34.9 | 0.322 | 0.636 |

What is claimed is:

1. A compound of the formula I 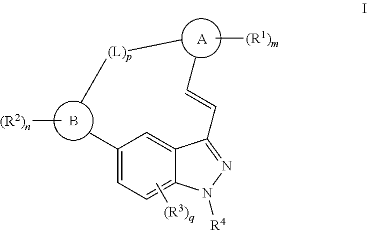
wherein
ring A is a 5- to 10-membered heteroarylene or C₆-C₁₀ arylene;
ring B is a 5-membered heteroarylene;
each L is independently —O—, —S—, —S(O)—, —S(O)₂—, —N(R⁵)C(O)—, —C(O)N(R⁵)—, —N(R⁵)—, —N(R⁵)S(O)—, —S(O)N(R⁵)—, —N(R⁵)S(O)₂—, —S(O)₂N(R⁵)—, or —C(R⁶)(R⁷)—, provided that (L)ₚ does not comprise an O—O, S—O, or N—N bond;
each R¹, R², and R³ when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
R⁴ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR′;
each R⁵, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{e}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{e}, —S(O)R^{e}, —S(O)₂Re, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{e}, —CN, or —NO₂;
each R⁶ and R⁷, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C1-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{c}R^{f}, —S(O)₂NR^{e}R^{f}, —NR^{e}R^{f}, —NR^{e}C(O)R^{f}, —NR^{c}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)ORe, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
or two of R¹, R⁶ and R⁷, taken together with the atom or atoms to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁵, R⁶ and R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C1-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C1-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C1-C₆ alkyl)₂, —OS(O)N(C₂-C₆ alkylene), —OS(O)₂N(H or C1-C₆ alkyl)₂, —OS(O)₂N(C₂-C₆ alkylene), —S(H or C₁-C₆ alkyl), —S(O)(H or C1-C₆ alkyl), —S(O)₂(H or C1-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C1-C₆ alkyl)C(O)—(H or C1-C₆ alkyl), —N(H or C1-C₆ alkyl)C(O)O(H or C₁-C₆ alkyl), —N(H or C₁-C₆ alkyl)C(O)N(H or C₁-C₆ alkyl)₂, —N(H or C1-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C1-C₆ alkyl)₂, —N(H or C1-C₆ alkyl)S(O)N(C2-C₆ alkylene), —N(H or C1-C₆ alkyl)S(O)₂N(H or C1-C₆ alkyl)₂, —N(H or C1-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C1-C₆ alkyl)₂, —P(O)(C₂-C₆ alkylene), —P(O)₂(H or C1-C₆ alkyl)₂, —P(O)₂(C₂-C₆ alkylene), —P(O)N(H or C1-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C1-C₆ alkyl), —P(O)₂O(H or C1-C₆ alkyl), —CN, or —NO₂;
m is 0, 1, 2, 3, or 4;
n is 0, 1, 2, or 3;
p is 4, 5, 6, 7,8 or 9; and
q is 0, 1, or 2;
or a pharmaceutically acceptable salt thereof;
provided that the compound is not of the formula 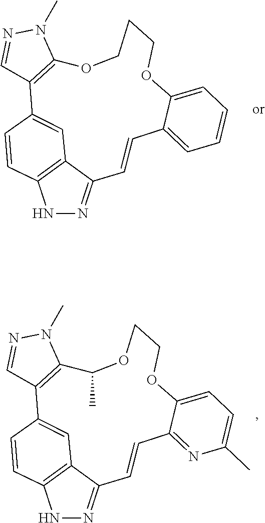 [SMILES: Cn1ncc2c1OCCCOc1ccccc1/C=C/c1n[nH]c3ccc-2cc13 ; Cc1ccc2c(n1)/C=C/c1n[nH]c3ccc(cc13)-c1cnn(C)c1[C@@H](C)OCCO2]
or a pharmaceutically acceptable salt thereof.

2. The compound of claim 1, or a pharmaceutically acceptable salt thereof, wherein (L)ₚ does not comprise a —NR⁵C(O)— directly covalently attached to ring A.

3. The compound of claim 1 or 2, or a pharmaceutically acceptable salt thereof, wherein ring A is a C₆-C₁₀ arylene, and m is 0, 1, or 2.

4. The compound of any one of the preceding claims, or a pharmaceutically acceptable salt thereof, wherein ring A is a phenylene, and m is 0, 1, or 2.

5. The compound of any one of the preceding claims, or a pharmaceutically acceptable salt thereof, wherein ring A is a phenylene, and m is 0 or 1.

6. The compound of any one of the preceding claims, or a pharmaceutically acceptable salt thereof, wherein ring A is a phenylene, m is 1, and R¹ is methyl, ethyl, F, Cl, Br, —CN, 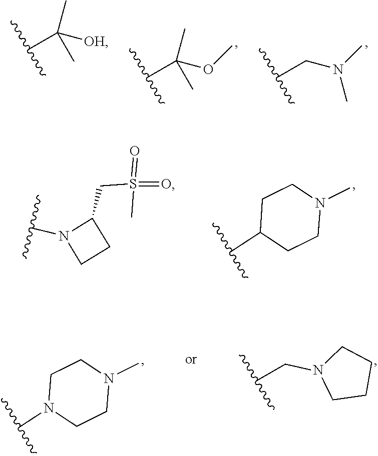
wherein each “ [symbol] ” represents a point of covalent attachment.

7. The compound of any one of the preceding claims, or a pharmaceutically acceptable salt thereof, wherein ring A is 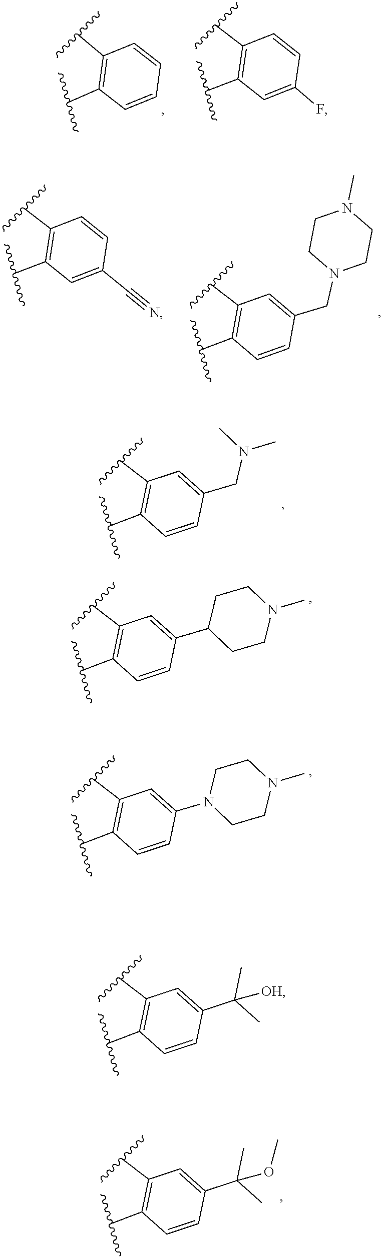 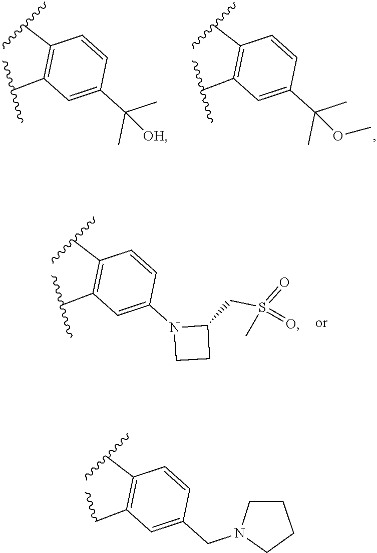
wherein each “ [symbol] ” represents a point of covalent attachment.

8. The compound of any one of claims 1 to 3, or a pharmaceutically acceptable salt thereof, wherein ring A is a 5- to 10-membered heteroarylene, and m is 0, 1, 2, or 3.

9. The compound of any one of claims 1 to 3, or a pharmaceutically acceptable salt thereof, wherein ring A is a 5- to 10-membered heteroarylene, and m is 0, 1, or 2.

10. The compound of any one of the claims 1 to 3, or 9, or a pharmaceutically acceptable salt thereof, having the formula II 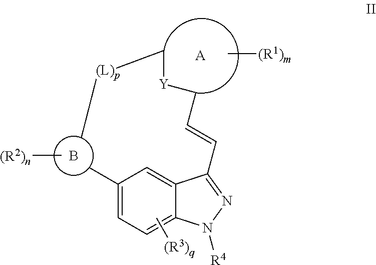
wherein Y is a single, a double bond, —O—, —S—, ═C(H)—, ═C(R⁵)—, —N(H)—, —N(R⁵)— or ═N—, and ring A is a 5- to 10-membered heteroarylene.

11. The compound of any one of claims 1 to 3, 9 or 10, or a pharmaceutically acceptable salt thereof, having the formula III 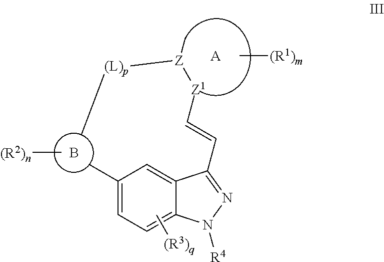
wherein Z is 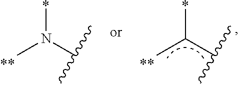
wherein * is a point of covalent attachment to (L)ₚ provided that * is not an N—O or N—N bond, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is 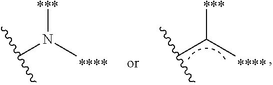
wherein *** is a point of covalent attachment to “ [symbol] ”, * is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, provided that both Z and Z¹ are not a nitrogen atom; and ring A is a 5- to 10-membered heteroarylene.

12. The compound of any one of claims 1 to 3, or 9 to 11, or a pharmaceutically acceptable salt thereof, having the formula IV 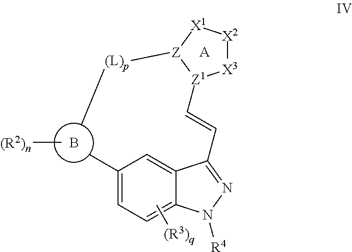
wherein
X¹, X², and X³ are each independently —O—, —S—, ═C(H)—, ═C(R¹)—, —N(H)—, —N(R¹)— or ═N—, provided that at least one of X¹, X², and X³ is not ═C(H)—, or ═C(R¹)—;
Z is 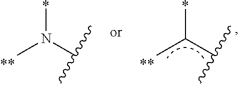
wherein * is a point of covalent attachment to (L)ₚ provided that * is not an N—O or N—N bond, ** is a point of covalent attachment to Z¹, “ [symbol] ” represents a point of covalent attachment to a ring atom of ring A, and “ [symbol] ” represents the condition that between the points of attachment ** and “ [symbol] ”, one bond is a single bond and one bond is a double bond; Z¹ is 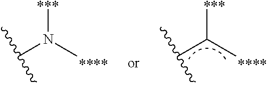
wherein *** is a point of covalent attachment to “ [symbol] ”, * is a point of covalent attachment to Z, “ [symbol] ” represents a point of covalent attachment to a ring atom of ring A, and “ [symbol] ” indicates the condition that between the points of attachment **** and “ [symbol] ”, one bond is a single bond and one bond is a double bond, provided that both Z and Z¹ are not a nitrogen atom; and
ring A is a 5- to 10-membered heteroarylene.

13. The compound of any one of the preceding claims, or a pharmaceutically acceptable salt thereof, wherein ring A is a 5- to 10-membered heteroarylene selected from the group consisting of 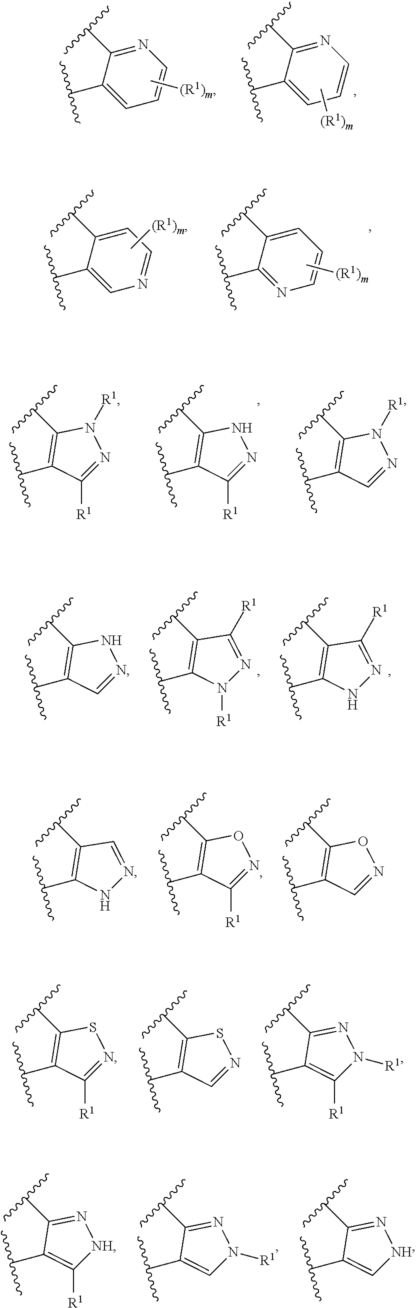 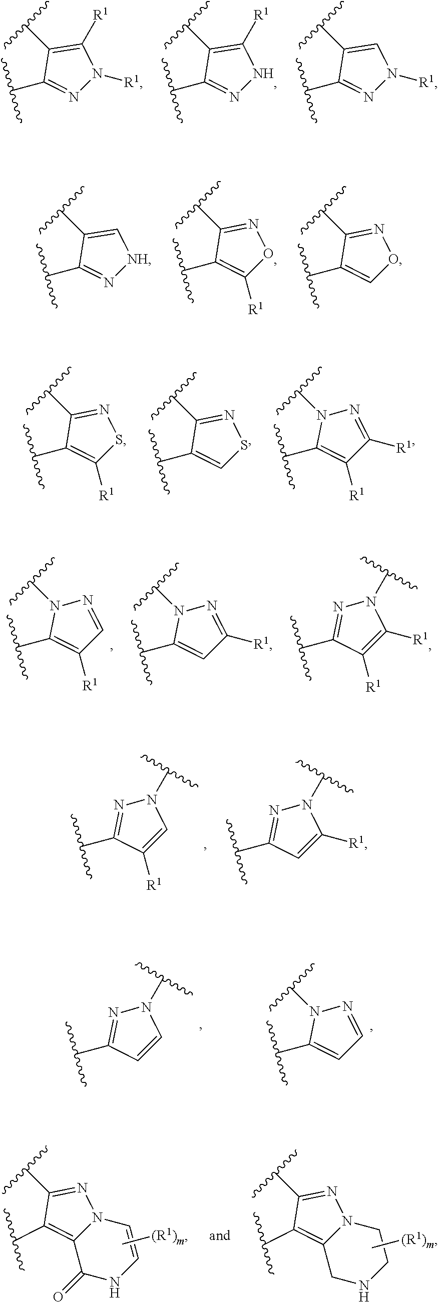
wherein each “ [symbol] ” represents a point of covalent attachment.

14. The compound of any one of the preceding claims, or a pharmaceutically acceptable salt thereof, wherein ring A is a 5- to 10-membered heteroarylene selected from the group consisting of 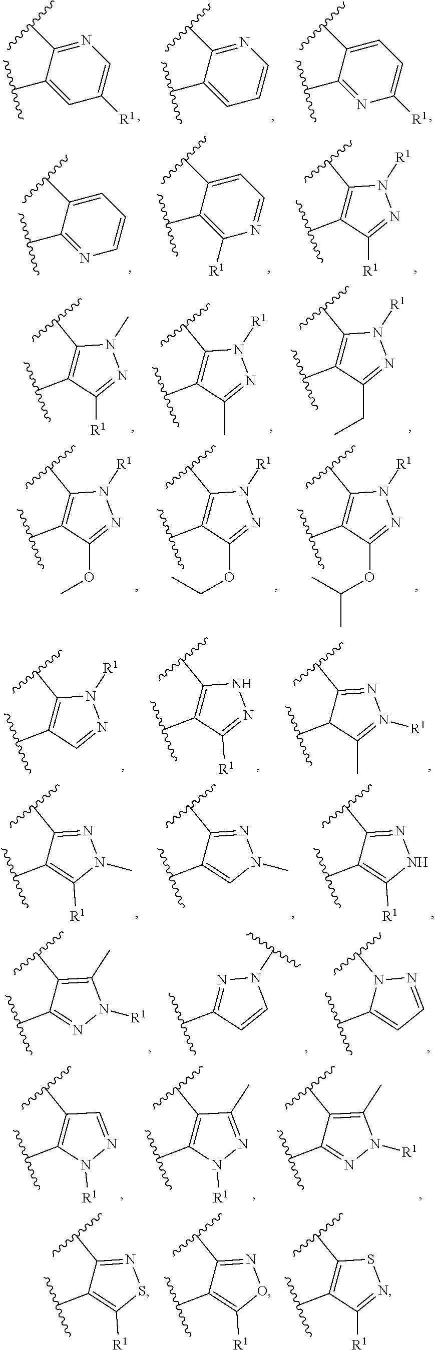 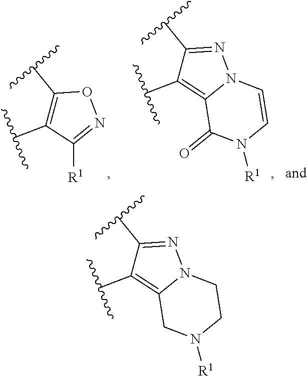
wherein each “ [symbol] ” represents a point of covalent attachment.

15. The compound of any one of the preceding claims, or a pharmaceutically acceptable salt thereof, wherein ring A is a 5- to 10-membered heteroarylene selected from the group consisting of 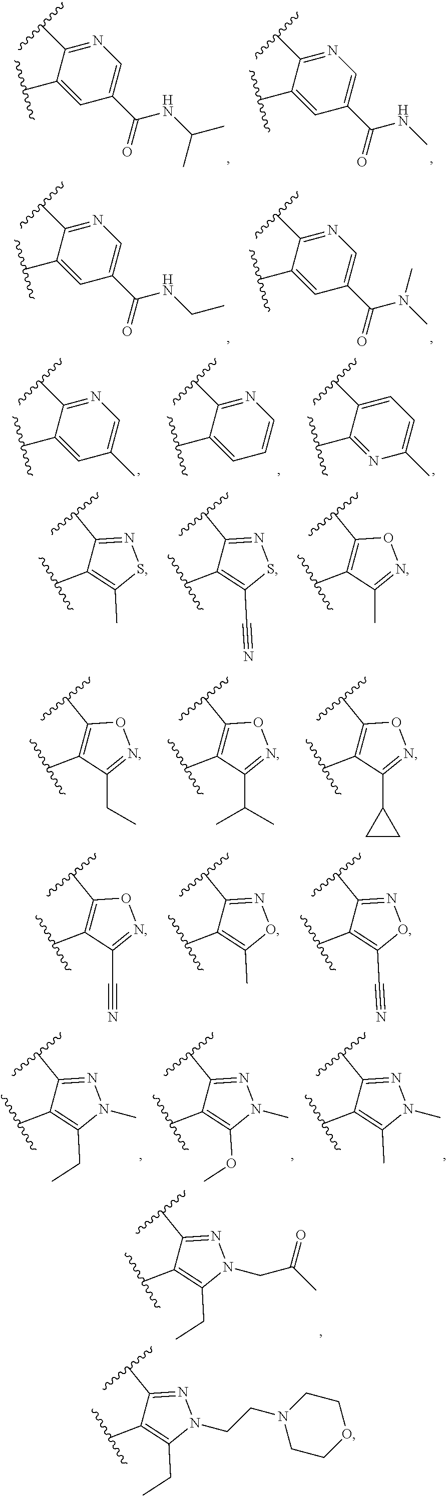 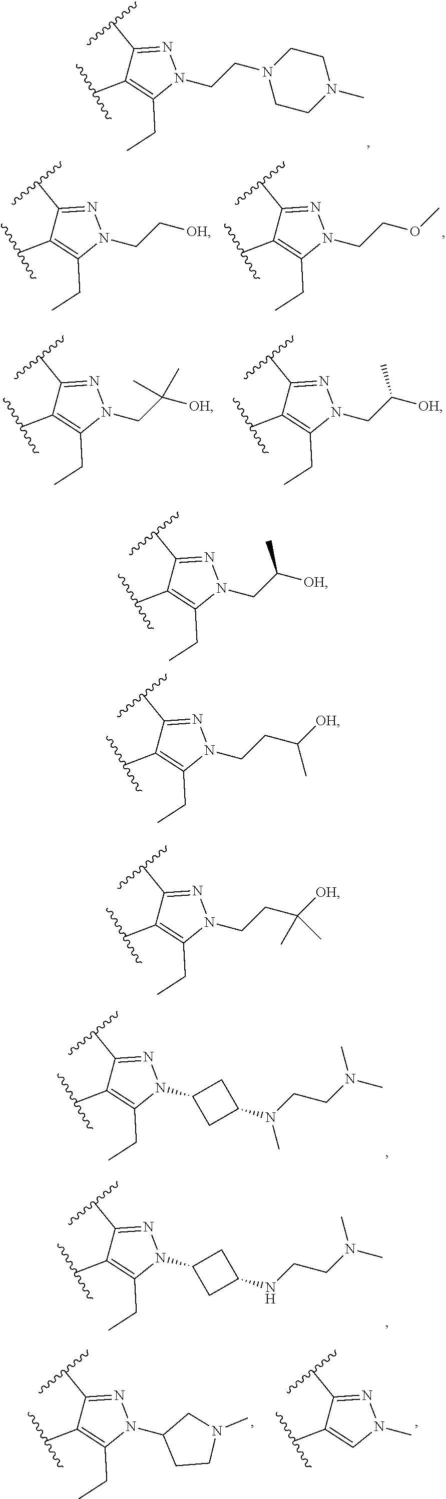 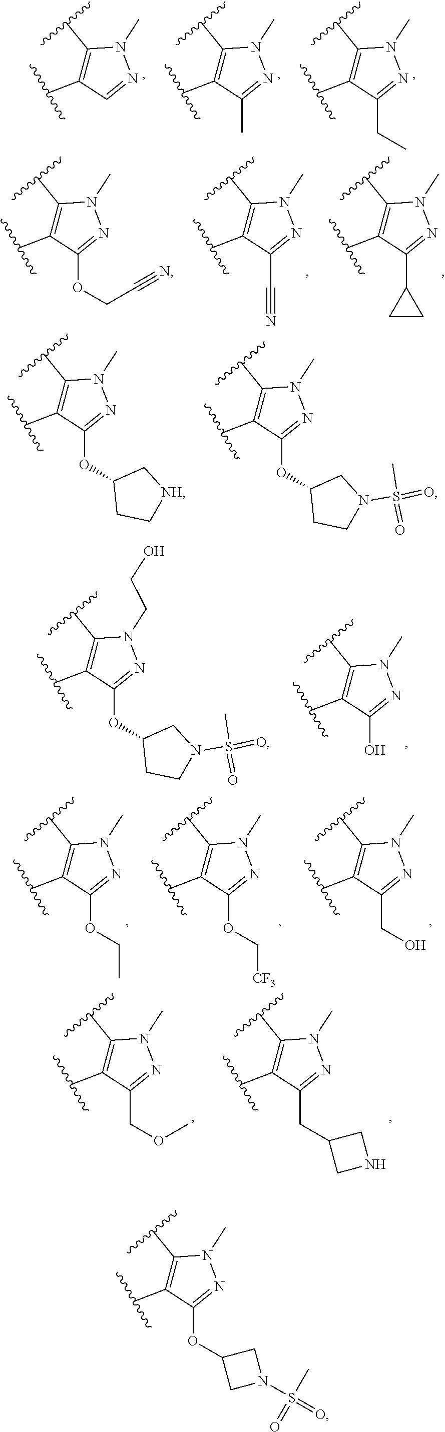 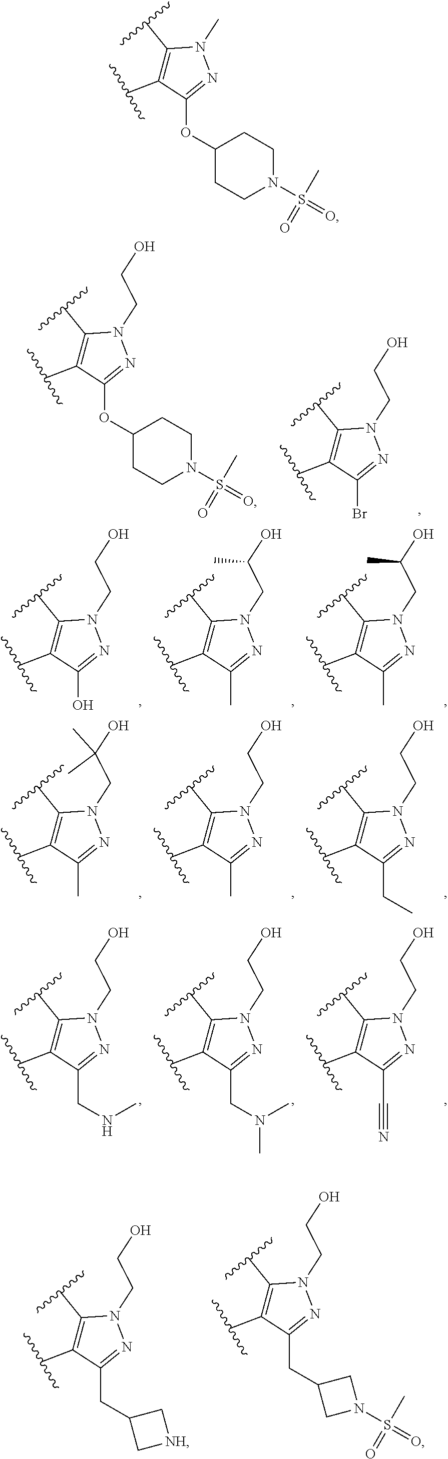 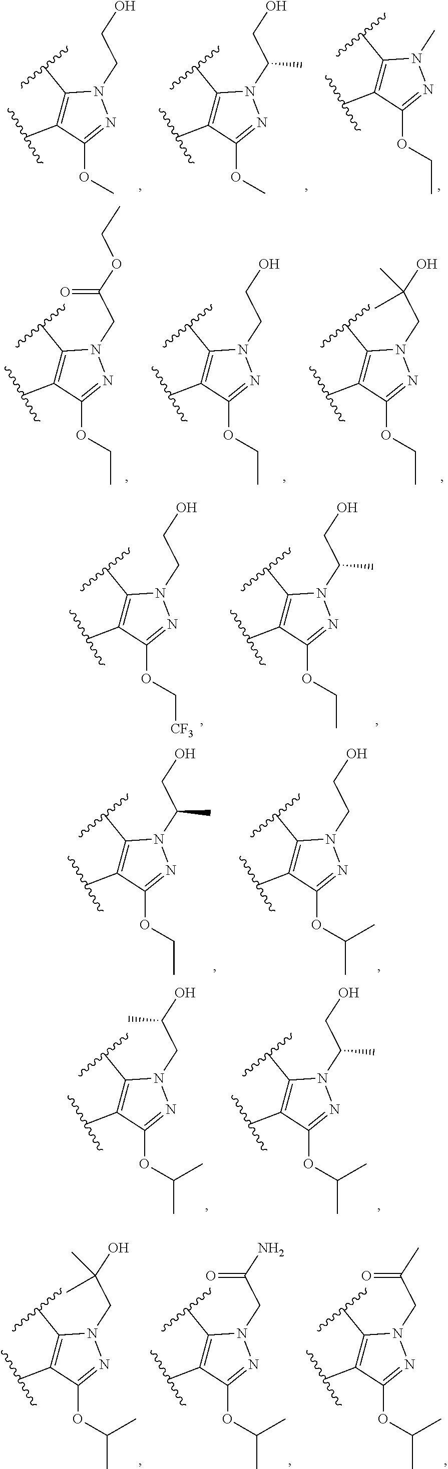 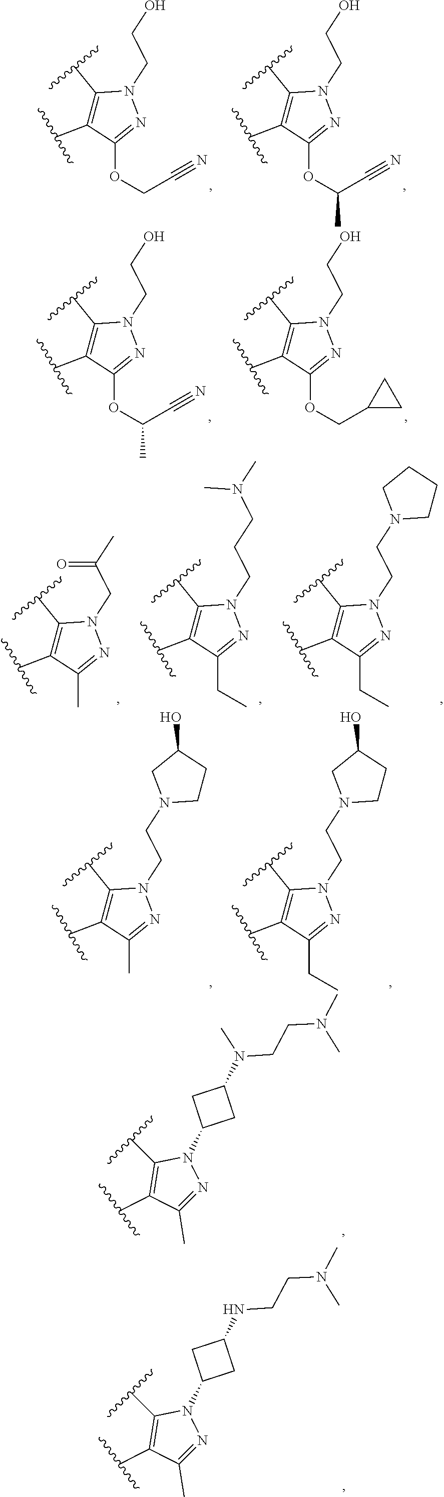 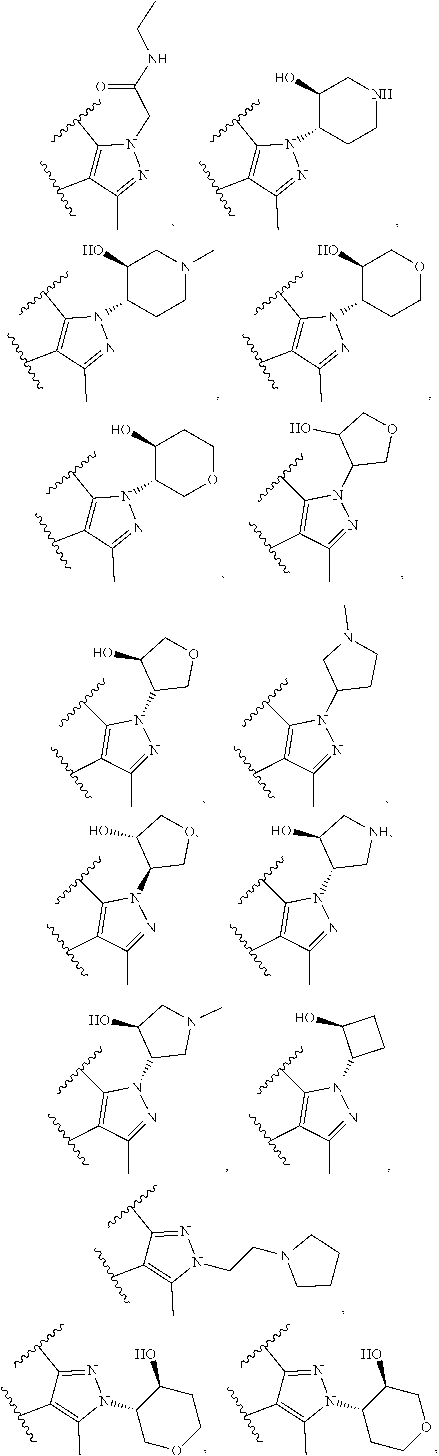 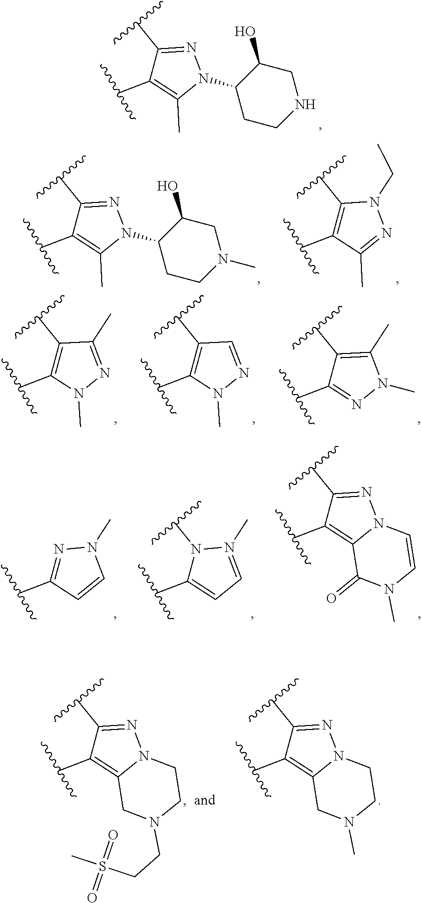
wherein each “ [symbol] ” represents a point of covalent attachment.

16. The compound of any one of the preceding claims, or a pharmaceutically acceptable salt thereof, wherein ring B is a 5-membered heteroarylene selected from the group consisting of 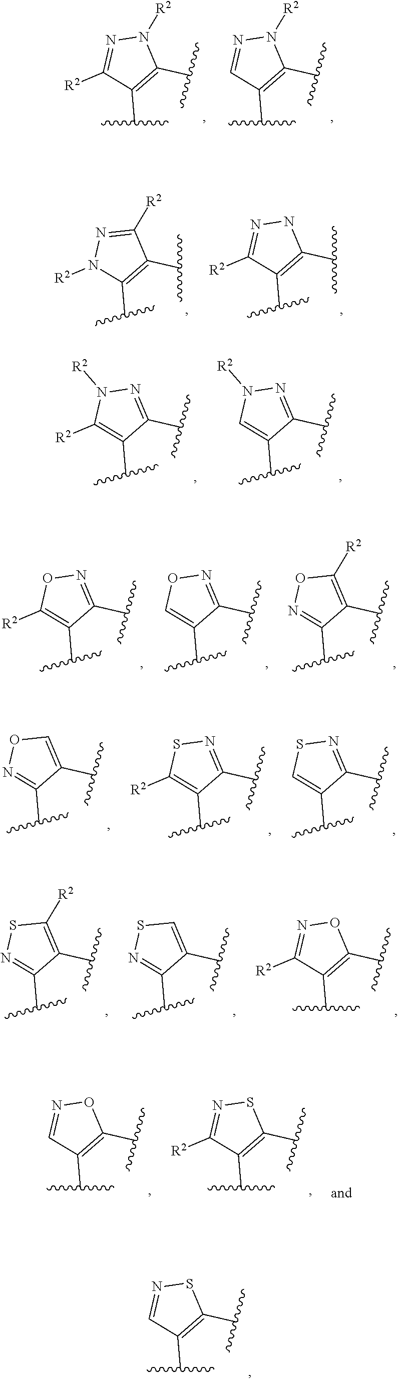
wherein each “ [symbol] ” represents a point of covalent attachment.

17. The compound of any one of the preceding claims, or a pharmaceutically acceptable salt thereof, wherein ring B is a 5-membered heteroarylene selected from the group consisting of 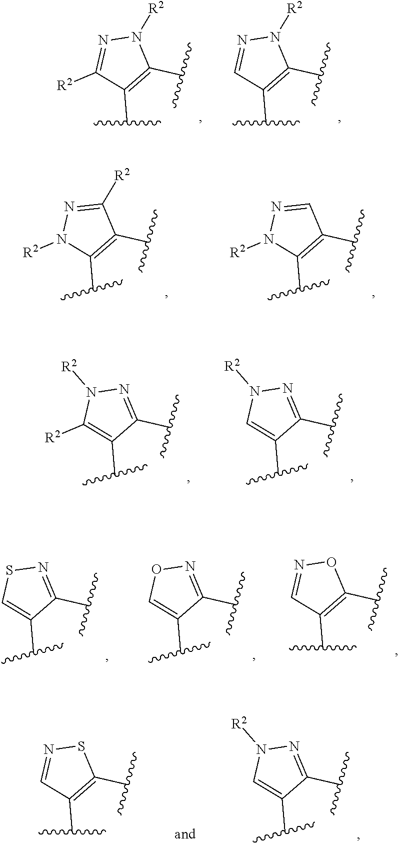
wherein each “ [symbol] ” represents a point of covalent attachment.

18. The compound of any one of the preceding claims, or a pharmaceutically acceptable salt thereof, wherein ring B is a 5-membered heteroarylene selected from the group consisting of 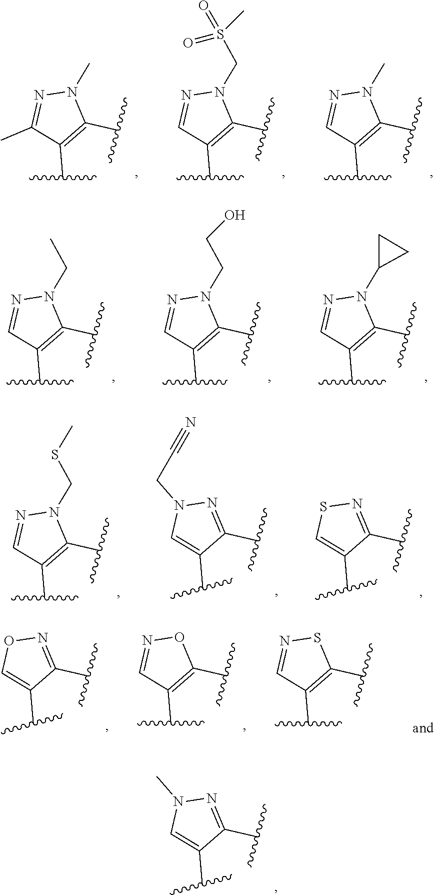
wherein each “ [symbol] ” represents a point of covalent attachment.

19. The compound of any one of the preceding claims, or a pharmaceutically acceptable salt thereof, q is 0, 1, or 2.

20. The compound of any one of the preceding claims, or a pharmaceutically acceptable salt thereof, q is 0 or 1.

21. The compound of any one of the preceding claims, or a pharmaceutically acceptable salt thereof, wherein R³ is F.

22. The compound of any one of the preceding claims, or a pharmaceutically acceptable salt thereof, wherein R⁴ is H or methyl.

23. The compound of any one of the preceding claims, or a pharmaceutically acceptable salt thereof, wherein each L is independently —C(R⁶)(R⁷)—, —C(O)—, —O—, or —N(R⁵)—, provided that (L)ₚ does not comprise a —O—O— or a —O—N(R⁵)— bond, and the point of covalent attachment of (L)ₚ to ring A does not form a —N—N— or a —O—N— bond.

24. The compound of any one of the preceding claims, or a pharmaceutically acceptable salt thereof, wherein -(L)ₚ- is —CR⁶R⁷—O(CR⁶R⁷)₂O—, —CR⁶R⁷—O(CR⁶R⁷)₃O—, —(CR⁶R⁷)C(O)N(R⁵)—(CR⁶R⁷)₂—, —(CR⁶R⁷)N(R⁵)C(O)—(CR⁶R⁷)₂—, —O(CR⁶R⁷)₂N(R⁵)C(O)—(CR⁶R⁷)O—, —N(R⁵)—C(O)(CR⁶R⁷)₂O(CR⁶R⁷)₂—, —CR⁶R⁷O(CR⁶R⁷)₂O—(CR⁶R⁷)₂, —O(CR⁶R⁷)₂₀(CR⁶R⁷)₂O—, —CR⁶R⁷O—CR⁶R⁷—C(O)N(R⁵)—(CR⁶R⁷)₂—, —(CR⁶R⁷)₃O(CR⁶R⁷)₂—, —(CR⁶R⁷)₂0(CR⁶R⁷)₃—, —CR⁶R⁷—N(R⁵)—(CR⁶R⁷)₄O—, —CR⁶R⁷—N(R⁵)—(CR⁶R⁷)₃O—, —CR⁶R⁷—N(R⁵)—(CR⁶R⁷)₂O—, —CR⁶R⁷—N(R⁵)—(CR⁶R⁷)₂—, —CR⁶R⁷—N(R⁵)—(CR⁶R⁷)₃O—, —CR⁶R⁷—N(R⁵)—(CR⁶R⁷)₃—, —O(CR⁶R⁷)₂O—CR⁶R⁷—, —O(CR⁶R⁷)₂O(CR⁶R⁷)₂—, —O(CR⁶R⁷)₂O(CR⁶R⁷)₃—, —(CR⁶R⁷)₂—N(R⁵)—(CR⁶R⁷)₃—, —O(CR⁶R⁷)₂—N(R⁵)—CR⁶R⁷—, —(CR⁶R⁷)₂—N(R⁵)—(CR⁶R⁷)₂—, —O—(CR⁶R⁷)₂—, —O—(CR⁶R⁷)₃—, —O—(CR⁶R⁷)₄—, —O—(CR⁶R⁷)₅—, —O—(CR⁶R⁷)₂O—, —O—(CR⁶R⁷)₃O—, —O—(CR⁶R⁷)₄O—, or —O—(CR⁶R⁷)₅O—.

25. The compound of any one of the preceding claims, or a pharmaceutically acceptable salt thereof, wherein each R⁵ is independently H, methyl, ethyl, —C(O)CH₃, or —C(O)CH₂CH₃; or R⁵, when present, and an R⁶ or R⁷, when present, taken together with the atom or atoms to which they are attached, combine to form a 3- to 7-membered heterocycloalkyl; wherein each hydrogen atom in the 3- to 7-membered heterocycloalkyl formed when R⁵ and an R⁶ or R⁷ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

26. The compound of any one of the preceding claims, or a pharmaceutically acceptable salt thereof, wherein an R⁶ and R⁷, when present, taken together with the atom or atoms to which they are attached, combine to form a 3- to 7-membered heterocycloalkyl; wherein each hydrogen atom in the 3- to 7-membered heterocycloalkyl formed when R⁶ and R⁷ are taken together is independently optionally substituted by —OR^{e}, —OC(O)R^{e}, —OC(O)NR^{c}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{c}, —CN, or —NO₂.

27. The compound of any one of the preceding claims, or a pharmaceutically acceptable salt thereof, wherein each R⁶, that is not taken together with R⁵ or an R⁷, is independently H or C₁-C₆ alkyl.

28. The compound of any one of the preceding claims, or a pharmaceutically acceptable salt thereof, wherein one R⁶, that is not taken together with R⁵ or an R⁷, is methyl, and the remaining R⁶ and R⁷ are H.

29. The compound of any one of the preceding claims, or a pharmaceutically acceptable salt thereof, wherein -(L)ₚ- is —O—(CH₂)₂O—CH₂—, —OC(H)(CH₃)—CH₂—O—CH₂—, —CH₂O—(CH₂)₂O—, —C(H)(CH₃)—O—(CH₂)₂O—, —CH₂N(H)—(CH₂)₂O—, —CH₂N(CH₃)—(CH₂)₂O—, —CH₂N(CH₂CH₃)—(CH₂)₂O—, —O(CH₂)₂N(H)CH₂—, —O(CH₂)₂N(CH₃)CH₂—, —OCH₂—C(H)(CH₃)—N(CH(CH₃)₂)CH₂—, —OCH₂—C(H)(CH₂F)—N(CH₃)CH₂—, —OCH₂—C(H)(CH₂CH₃)—N(CH₃)CH₂—, —O(CH₂)₂N(C(O)CH₃)CH₂—, —OC(H)(CH₃)CH₂N(H)CH₂—, —OC(H)(CH₃)CH₂N(CH₃)CH₂—, —OC(H)(CH₂CN)CH₂N(CH₃)CH₂—, —OC(H)(CH₂CH₃)CH₂N(CH₃)CH₂—, —OC(H)(CH₂F)CH₂N(CH₃)CH₂—, —OC(H)(CHF₂)CH₂N(CH₃)CH₂—, —OC(H)(CH₂OH)CH₂N(CH₃)CH₂—, —OC(H)(CH₃)C(O)N(CH₃)CH₂—, —OC(H)(cyclobutyl)CH₂N(CH₃)CH₂—, —OC(H)(CH₃)CH₂N(CH₂CH₃)CH₂—, —OC(H)(CH₃)CH₂N(CH(CH₃)₂)—CH₂—, —OC(H)(CH₃)CH₂N(CH₃)—C(H)(CH₃)—, —OC(H)(CH₃)CH₂N(CH(oxetan-3-yl)-CH₂—, —OC(H)(CH₃)CH₂N(cyclopropyl)-CH₂—, —OCH₂C(H)(CH₃)N(cyclopropyl)-CH₂—, —OC(H)(CH₃)CH₂N(C(O)CH₃)CH₂—, —O(C(H)(CH₃))CH₂N(C(O)CH₂CH₃)CH₂—, —CH₂N(H)—(CH₂)₂(C(H)CH₃)CH₂O—, —CH₂N(CH₃)—C(H)(CH₃)—CH₂O—, —CH₂N(CH₃)—(CH₂)₂(C(H)CH₃)CH₂O—, —CH₂N(CH₃)—(CH₂)—(C(H)CH₃)O—, —O(CH₂)₂—, —O(CH₂)₃—, —O(CH₂)₄—, —O(CH₂)₃O—, —O(CH₂)₄O—, —O(CH₂)₅O—, —O—(C(H)(CH₃)—(CH₂)₂O—, —O(CH₂)₂—C(H)(CH₃)—O—, —O(CH₂)₂C(H)(CH₃)CH₂O—, —O(CH₂)₃C(H)(CH₃)CH₂O—, —O(CH₂)₂N(H)C(O)—(CH₂)O—, —O(CH₂)₂N(CH₃)C(O)—(CH₂)O—, —O(CH₂)₂O(CH₂)₂O—, —OC(H)(CH₃)CH₂OCH₂—, —O(CH₂)₂OCH₂C(H)(CH₃)O—, —O(CH₂)₂OC(H)(CH₃)—CH₂O—,

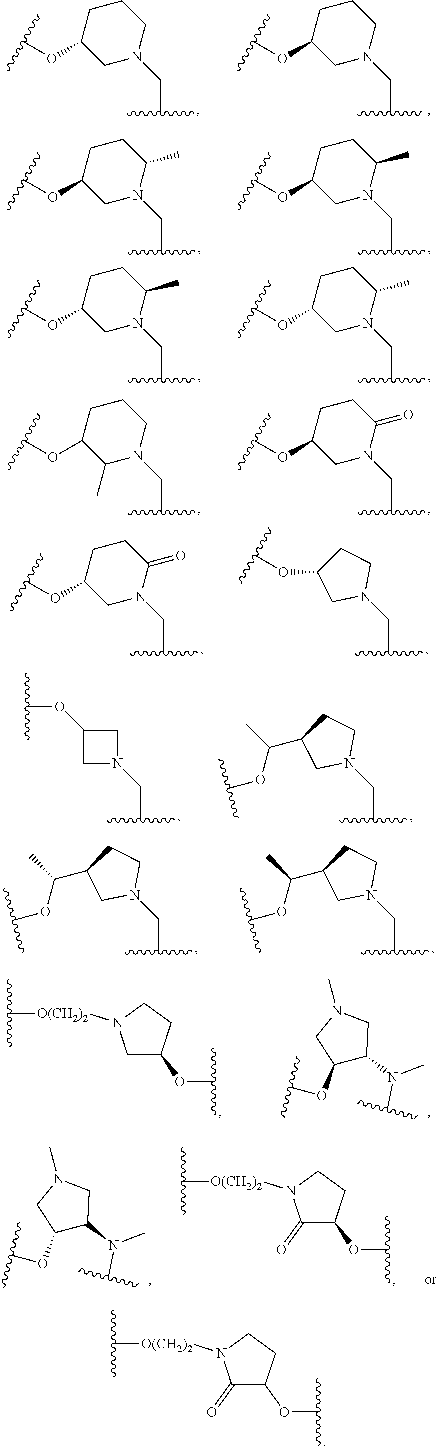

30. A compound selected from the group consisting of (11E)-1-methyl-1,18,19,21-tetrahydro-8H-10,7,4-(ethan[1]yl[1,2]diylidene)pyrazolo[3,4-f][1,4,12,13]benzodioxadiazacyclooctadecine;
(11E)-1-[(methanesulfonyl)methyl]-19,20-dihydro-1H,8H,18H-10,7,4-(ethan[1]yl[1,2]diylidene)pyrazolo[3,4-f][1,5,12,13]benzodioxadiazacyclooctadecine;
(11E)-1-methyl-18,19,20,21-tetrahydro-1H,8H-10,7,4-(ethan[1]yl[1,2]diylidene)pyrazolo[3,4-g][1,6,13,14]benzodioxadiazacyclononadecine;
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-[(4-methylpiperazin-1-yl)methyl]-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-[(dimethylamino)methyl]-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(1-methylpiperidin-4-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-13-(2-methoxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-3-methyl-13-(4-methylpiperazin-1-yl)-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-fluoro-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-(2-hydroxypropan-2-yl)-3-methyl-3,5,6,9,10,19-hexahydro-8H-20,22-etheno-7,10-methanodipyrazolo[3,4-i:4′,3′-m][1,8,5]benzodioxazacyclooctadecin-25-one;
(10R,16E)-14-{(2S)-2-[(methanesulfonyl)methyl]azetidin-1-yl}-3-methyl-3,5,6,9,10,19-hexahydro-8H-20,22-etheno-7,10-methanodipyrazolo[3,4-i:4′,3′-mi][1,8,5]benzodioxazacyclooctadecin-25-one;
(10R,16E)-3-methyl-25-oxo-3,5,6,9,10,19-hexahydro-8H-20,22-etheno-7,10-methanodipyrazolo|3,4-i:4′,3′-m∥1,8,5|benzodioxazacyclooctadecine-14-carbonitrile; and
(10R,16E)-3-methyl-3,5,6,9,10,19-hexahydro-8H-20,22-etheno-7,10-methanodipyrazolo[3,4-i:4′,3′-m][1,8,5]benzodioxazacyclooctadecine;
or a pharmaceutically acceptable salt thereof.

31. A compound selected from the group consisting of (18E)-8-methyl-N-(propan-2-yl)-8,9,11,12-tetrahydro-2H-3,5-ethenodipyrazolo [3′,4′:9,10;4″,3″:13,14][1,4]dioxacyclopentadecino[5,6-b]pyridine-16-carboxamide;
(18E)-N,8-dimethyl-8,9,11,12-tetrahydro-2H-3,5-ethenodipyrazolo [3′,4′:9,10;4″,3″:13,14][1,4]dioxacyclopentadecino[5,6-b]pyridine-16-carboxamide;
(18E)-N,N,8-trimethyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(18E)-N-ethyl-8-methyl-8,9,11,12-tetrahydro-2H-3,5-ethenodipyrazolo[3′,4′:9,10;4″,3″:13,14][1,4]dioxacyclopentadecino[5,6-b]pyridine-16-carboxamide;
(10S,18E)-8,10,16-trimethyl-2,8,11,12-tetrahydro-10H-3,5-ethenodipyrazolo[3′,4′:10,11;4″,3″:14,15][1,5]dioxacyclopentadecino[7,6-b]pyridine;
(10S,18E)-17-ethoxy-8,10,12-trimethyl-2,8,10,11,12,13-hexahydro-3,5-ethenodipyrazolo[3,4-j:4′,3′-n]pyrido[4,3-f][1,4]oxazacyclopentadecine; and
(10S,18E)-16-ethoxy-8,10,12-trimethyl-2,8,10,11,12,13-hexahydro-3,5-ethenodipyrazolo[3,4-j:4′,3′-n]pyrido[3,2-][1,4]oxazacyclopentadecine;
or a pharmaceutically acceptable salt thereof.

32. A compound selected from the group consisting of (17E)-8,14,16-trimethyl-2,8,9,11,12,14-hexahydro-3,5-etheno[1,4]dioxacyclopentadecino[11,10-c:15,14-c′:6,7-c″]tripyrazole;
2-[(17E)-8,9,16-trimethyl-8,9,11,12-tetrahydro-3,5-etheno[1,4]dioxacyclopentadecino[11,10-c:15,14-c′:6,7-c′]tripyrazol-14(2H)-yl]ethan-1-ol;
(17E)-8,14,16-trimethyl-2,11,12,14-tetrahydro-8H,10H-3,5-etheno[1,5]dioxacyclopentadecino[10,11-c:15,14-c′:6,7-c″]tripyrazole;
(17E)-19-fluoro-8,14,16-trimethyl-2,11,12,14-tetrahydro-8H,10H-3,5-etheno[1,5]dioxacyclopentadecino[10,11-c:15,14-c′:6,7-c″]tripyrazole;
(17E)-16-ethyl-8,14-dimethyl-2,11,12,14-tetrahydro-8H,10H-3,5-etheno[1,5]dioxacyclopentadecino[10,11-c:15,14-c′:6,7-c″]tripyrazole;
1-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-2-methylpropan-2-ol;
1-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]propan-2-one;
2-[(17E)-8,16-dimethyl-2,8,11,12-tetrahydro-10H,14H-3.5-etheno[1,5]dioxacyclopentadecino[10,11-c:15,14-c′:6,7-c″]tripyrazol-14-yl]ethan-1-ol;
(17E)-8,16-dimethyl-14-[2-(pyrrolidin-1-yl)ethyl]-2,11,12,14-tetrahydro-8H,10H-3,5-etheno[1,5]dioxacyclopentadecino[10,11-c:15,14-c′:6,7-c″]tripyrazole;
(3S)-1-{2-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]ethyl}pyrrolidin-3-ol;
(17E)-16-ethyl-8,12,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-8,10,14,16-tetramethyl-2,11,12,14-tetrahydro-8H,10H-3,5-etheno[1,5]dioxacyclopentadecino[10,11-c:15,14-c′:6,7-c″]tripyrazole;
(10R,17E)-8,10,14,16-tetramethyl-2,11,12,14-tetrahydro-8H,10H-3,5-etheno[1,5]dioxacyclopentadecino[10,11-c:15,14-c′:6,7-c″]tripyrazole;
(12S,17E)-16-ethyl-8,12,14-trimethyl-2,11,12,14-tetrahydro-8H,10H-3,5-etheno[1,5]dioxacyclopentadecino[10,11-c:15,14-c′:6,7-c″]tripyrazole;
(12R,17E)-16-ethyl-8,12,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-16-ethyl-8,10,14-trimethyl-2,11,12,14-tetrahydro-8H,10H-3,5-etheno[1,5]dioxacyclopentadecino[10,11-c:15,14-c′:6,7-c″]tripyrazole;
(10S,17E)-16-cyclopropyl-8,10,14-trimethyl-2,11,12,14-tetrahydro-8H,10H-3,5-etheno[1,5]dioxacyclopentadecino[10,11-c:15,14-c′:6,7-c″]tripyrazole;
(17E)-16-(methoxymethyl)-8,14-dimethyl-2,11,12,14-tetrahydro-8H,10H-3,5-etheno[1,5]dioxacyclopentadecino[10,11-c:15,14-c′:6,7-c″]tripyrazole;
(17E)-8,14,16-trimethyl-2,10,11,12,13,14-hexahydro-8H-3,5-ethenotripyrazolo[3,4-f:3′,4′-j:4″,3″-n][1,4]oxazacyclopentadecine;
1-|(17E)-8,14,16-trimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one;
(17E)-8,12,14,16-tetramethyl-2,10,11,12,13,14-hexahydro-8H-3,5-ethenotripyrazolo[3,4-f:3′,4′-j:4″,3″-n][1,4]oxazacyclopentadecine;
(17E)-6,8,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(17E)-16-ethyl-8,12,14-trimethyl-2,10,11,12,13,14-hexahydro-8H-3,5-ethenotripyrazolo[3,4-f:3′,4′-j:4″,3″-n][1,4]oxazacyclopentadecine;
(17E)-7,14,16-trimethyl-2,10,11,12,13,14-hexahydro-7H-3,5-ethenotripyrazolo[3,4-f:3′,4′-j:4″,3″-n][1,4]oxazacyclopentadecine;
(17E)-7,12,14,16-tetramethyl-2,10,11,12,13,14-hexahydro-7H-3,5-ethenotripyrazolo [3,4-f:3′,4′-j:4″,3″-n][1,4]oxazacyclopentadecine;
2-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(17E)-8,14,16-trimethyl-2,9,10,11,12,14-hexahydro-8H-3,5-ethenotripyrazolo[3,4-f:4′,3′-j:4″,3″-n][1,4]oxazacyclopentadecine;
(17E)-8,10,14,16-tetramethyl-2,9,10,11,12,14-hexahydro-8H-3,5-ethenotripyrazolo[3,4-f:4′,3′-j:4″,3″-n][1,4]oxazacyclopentadecine;
2-[(17E)-8,16-dimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
2-[(17E)-10-ethyl-8,16-dimethyl-2,8,9,10,11,12-hexahydro-14H-3,5-ethenotripyrazolo[3,4-f:4′,3′-j:4″,3″-n][1,4]oxazacyclopentadecin-14-yl]ethan-1-ol;
(19E)-8,13,16,18-tetramethyl-2,11,12,13,14,16-hexahydro-8H,10H-3,5-etheno[1,7]dioxacycloheptadecino[12,13-c:17,16-c′:8,9-c″]tripyrazole;
2-[(19E)-8,13,18-trimethyl-2,8,11,12,13,14-hexahydro-10H,16H-3,5-etheno[1,7]dioxacycloheptadecino[12,13-c:17,16-c′:8,9-c″]tripyrazol-16-yl]ethan-1-ol;
(19E)-8,16,18-trimethyl-2,11,12,16-tetrahydro-8H,10H-3,5-ethenotripyrazolo[3,4-h:3′,4′-l:4″,3″-p][1,7,4]dioxazacycloheptadecin-13(14H)-one;
(19E)-8,12,16,18-tetramethyl-2,11,12,16-tetrahydro-8H,10H-3,5-ethenotripyrazolo [3,4-h:3′,4′-l:4″,3″-p][1,7,4]dioxazacycloheptadecin-13(14H)-one;
(13R,19E)-8,13,16,18-tetramethyl-2,10,11,13,14,16-hexahydro-8H-3,5-etheno[1,4,7]trioxacycloheptadecino[12,13-c:17,16-c′:8,9-c″]tripyrazole;
(13S,19E)-8,13,16,18-tetramethyl-2,10,11,13,14,16-hexahydro-8H-3,5-ethenol[1,4,7]trioxacycloheptadecinol[12,13-c:17,16-c′:8,9-c″]tripyrazole;
2-[(19E)-8,13,18-trimethyl-2,8,10,11,13,14-hexahydro-16H-3,5-etheno[1,4,7]trioxacycloheptadecino[12,13-c:17,16-c′:8,9-c″]tripyrazol-16-yl]ethan-1-ol;
(19E)-8,13,18-trimethyl-16-[2-(pyrrolidin-1-yl)ethyl]-2,10,11,13,14,16-hexahydro-8H-3,5-etheno[1,4,7]trioxacycloheptadecino[12,13-c:17,16-c′:8,9-c′]tripyrazole;
(3S)-1-{2-[(17E)-16-ethyl-8-methyl-2,8,11,12-tetrahydro-10H,14H-3,5-etheno[1,5]dioxacyclopentadecino[10,11-c:15,14-c′:6,7-c″]tripyrazol-14-yl]ethyl}pyrrolidin-3-ol;
(19E)-22-fluoro-8,16,18-trimethyl-2,10,11,13,14,16-hexahydro-8H-3,5-etheno[1,4,7]trioxacycloheptadecino[12,13-c:17,16-c′:8,9-c ″]tripyrazole;
3-[(17E)-16-ethyl-8-methyl-2,8,11,12-tetrahydro-10H,14H-3,5-etheno[1,5]dioxacyclopentadecino[10,11-c:15,14-c:6,7-c″]tripyrazol-14-yl]-N,N-dimethylpropan-1-amine;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]pyrrolidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-1-methylpyrrolidin-3-ol;
(3R,4S)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]oxolan-3-ol;
(3S,4R)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]oxolan-3-ol;
(rac)-2-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]cyclobutan-1-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]piperidin-3-ol;
(rac)-4-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-1-methylpiperidin-3-ol;
(rac)-1,5-anhydr-3o-2,3-dideoxy-3-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-L-threo-pentitol;
(rac)-1,5-anhydro-2,4-dideoxy-2-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]-L-threo-pentitol;
(12R,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(12S,17E)-8,10,12,14,16-pentamethyl-2,9,10,11,12,14-hexahydro-8H-3,5-ethenotripyrazolo[3,4-f:4′,3′-j:4″,3″-n][1,4]oxazacyclopentadecine;
(rac)-(3S,4S)-1-methyl-4-[(17E)-8,10,16-trimethyl-2,8,9,10,11,12-hexahydro-14H-3,5-ethenotripyrazolo[3,4-f:4′,3′-j:4″,3″-n][1,4]oxazacyclopentadecin-14-yl]pyrrolidin-3-ol;
(11S,17E)-8,11,14,16-tetramethyl-2,9,10,11,12,14-hexahydro-8H-3,5-ethenotripyrazolo[3,4-f:4′,3′-j:4″,3″-n][1,4]oxazacyclopentadecine;
(17E)-8,9,16-trimethyl-14-[2-(pyrrolidin-1-yl)ethyl]-2,8,9,11,12,14-hexahydro-3,5-etheno[1,4]dioxacyclopentadecino[11,10-c:15,14-c′:6,7-c″]tripyrazole;
[(10S,17E)-8,10,14-trimethyl-2,11,12,14-tetrahydro-8H,10H-3,5-etheno[1,5]dioxacyclopentadecino[10,11-c:15,14-c′:6,7-c″]tripyrazol-16-yl]methanol;
N¹—{(1s,3s)-3-[(17E)-8,16-dimethyl-2,8,11,12-tetrahydro-10H,14H-3,5-etheno[1,5]dioxacyclopentadecinoo[10,11-c:15,14-c′:6,7-c″]tripyrazol-14-yl]cyclobutyl}-N¹,N′—,N²-trimethylethane-1,2-diamine;
(11S,17E)-8,10,11,14,16-pentamethyl-2,9,10,11,12,14-hexahydro-8H-3,5-ethenotripyrazolo[3,4-f:4′,3′-j:4″,3″-n][1,4]oxazacyclopentadecine;
N²—{(1s,3s)-3-[(17E)-8,16-dimethyl-2,8,11,12-tetrahydro-10H,14H-3,5-etheno[1,5]dioxacyclopentadecino[10,11-c:15,14-c′:6,7-c″]tripyrazol-14-yl]cyclobutyl}-N¹,N¹-dimethylethane-1,2-diamine;
(10R,15E)-3,12,14-trimethyl-5,6,9,10,12,18-hexahydro-3H,8H-19,21-etheno-7,10-methanotripyrazolo[3,4-i:3′,4′-m:4″,3″-q][1,8,4]dioxazacyclooctadecin-24-one;
(17E)-16-ethyl-8,10,14-trimethyl-2,9,10,11,12,14-hexahydro-8H-3,5-ethenotripyrazolo[3,4-f:4′,3′-j:4″,3″-n][1,4]oxazacyclopentadecine;
(17E)-10-ethyl-8,14,16-trimethyl-2,9,10,11,12,14-hexahydro-8H-3,5-ethenotripyrazolo[3,4-f:4′,3′-j:4″,3″-n][1,4]oxazacyclopentadecine;
2-[(10S,17E)-8,10,16-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-19-fluoro-8,10,12,16-tetramethyl-2,8,10,11,12,13-hexahydro-14H-3,5-ethenotripyrazolo[3,4f:3′,4′-j:4″,3″-n][1,4]oxazacyclopentadecin-14-yl]ethan-1-ol;
(17E)-8,10,16-trimethyl-14-(1-methylpyrrolidin-3-yl)-2,9,10,11,12,14-hexahydro-8H-3,5-ethenotripyrazolo[3,4-f:4′,3′-j:4″,3″-n][1,4]oxazacyclopentadecine;
2-[(10S,17E)-16-ethoxy-8,10,12-trimethyl-2,8,10,11,12,13-hexahydro-14H-3,5-ethenotripyrazolo[3,4f 3′,4′-j:4″,3″-n][1,4]oxazacyclopentadecin-14-yl]ethan-1-ol;
(10S,17E)-8,10,12,14,16-pentamethyl-2,10,11,12,13,14-hexahydro-8H-3,5-ethenotripyrazolo[3,4-f:3′,4′-j:4″,3″-n][1,4]oxazacyclopentadecine;
(10R,19E)-8,16,18-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(2S)-1-[(10S,17E)-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]propan-2-ol;
2-[(10S,17E)-19-chloro-8,10,12,16-tetramethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2R)-1-[(10S,17E)-8,10,12,16-tetramethyl-2,8,10,11,12,13-hexahydro-14H-3,5-ethenotripyrazolo[3,4f:3′,4′-j:4″,3″-n][1,4]oxazacyclopentadecin-14-yl]propan-2-ol;
2-[(10S,17E)-16-ethyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-ethyl-8,10,16-trimethyl-2,8,10,11,12,13-hexahydro-14H-3,5-ethenotripyrazolo[3,4f:3′,4′-j:4″,3″-n][1,4]oxazacyclopentadecin-14-yl]ethan-1-ol;
(10S,17E)-8,10,12,14-tetramethyl-16-[(piperidin-4-yl)oxy]-2,10,11,12,13,14-hexahydro-8H-3,5-ethenotripyrazolo[3,4-f:3′,4′-j:4″,3″-n][1,4]oxazacyclopentadecine;
2-[(10R,19E)-18-ethoxy-8-methyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[10S, 17E)-6,8,10,12,16-pentamethyl-2, 8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,14,16-tetramethyl-12-(oxetan-3-yl)-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-{[1-(methanesulfonyl)piperidin-4-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,13R,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
(10S,13S,17E)-8,10,12,13,14,16-hexamethyl-2,10,11,12,13,14-hexahydro-8H-3,5-ethenotripyrazolo[3,4-f:3′,4′-j:4″,3″-n][1,4]oxazacyclopentadecine;
2-{(10S,17E)-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-[(azetidin-3-yl)oxy]-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-16-{|1-(methanesulfonyl)azetidin-3-yl|oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(17E)-10-cyclobutyl-8,12,16-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-8,10,12,14-tetramethyl-16-{[(3S)-pyrrolidin-3-yl]oxy}-2,10,11,12,13,14-hexahydro-8H-3,5-ethenotripyrazolo[3,4-f:3′,4′-j:4″,3″-n][1,4]oxazacyclopentadecine;
(10S,17E)-16- {[(3S)-1-(methanesulfonyl)pyrrolidin-3-yl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(2S)-2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(10S,17E)-16-ethoxy-8,10,14-trimethyl-12-(propan-2-yl)-2,10,11,12,13,14-hexahydro-8H-3,5-ethenotripyrazolo[3,4-f:3′,4′-j:4″,3″-n][1,4]oxazacyclopentadecine;
(10S,17E)-8,10,14-trimethyl-2,11,12,14-tetrahydro-8H,10H-3,5-etheno[1,5]dioxacyclopentadecino[10,11-c:15,14-c′:6,7-c″]tripyrazole-16-carbonitrile;
(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10R,19E)-18-ethoxy-8,16-dimethyl-2,8,10,11,12,13,15,16-octahydro-3,5-etheno-10,14-methanotripyrazolo[3,4-h:3′,4′-1:4″,3″-p][1, 6]oxazacycloheptadecine;
(10S,17E)-12-cyclopropyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
(10S,17E)-8,10,12,14-tetramethyl-16-(2,2,2-trifluoroethoxy)-2,10,11,12,13,14-hexahydro-8H-3,5-ethenotripyrazolo[3,4-f:3′,4′-j:4″,3″-n][1,4]oxazacyclopentadecine;
{[(10S,17E)-8,10,12,14-tetramethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
(10S,17E)-10,14,16-trimethyl-8-[(methylsulfanyl)methyl]-12-(propan-2-yl)-2,10,11,12,13,14-hexahydro-8H-3,5-ethenotripyrazolo[3,4-f:3′,4′-j:4″,3″-n][1,4]oxazacyclopentadecine;
(10S,17E)-12-ethyl-8,10,14,16-tetramethyl-2,10,11,12,13,14-hexahydro-8H-3,5-ethenotripyrazolo[3,4-f:3′,4′-j:4″,3″-n][1,4]oxazacyclopentadecine;
2-[(10S,17E)-16-ethoxy-8-ethyl-10,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-8-ethyl-10,12-dimethyl-16-|(propan-2-yl)oxyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2R)-2-[(10S,17E)-16-ethoxy-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-19-fluoro-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-{[1-(methanesulfonyl)piperidin-4-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,19E)-8,16,18-trimethyl-2,8,10,11,12,13,15,16-octahydro-3,5-etheno-10,14-methanotripyrazolo[3,4-h:3′,4′-j:4″,3″-p][1,6]oxazacycloheptadecine;
2-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10S,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10S,17E)-16-{[(3S)-1-(methanesulfonyl)pyrrolidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-bromo-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2- {(10S,17E)-16-[(azetidin-3-yl)oxy]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-16-{[3-(methanesulfonyl)cyclobutyl]oxy}-8,10,12,14-tetramethyl-2,10,11,12,13,14-hexahydro-8H-3,5-ethenotripyrazolo[3,4-f:3′,4′-j:4″,3″-n][1,4]oxazacyclopentadecine;
(10S,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
(10R,19E)-18-ethoxy-8,16-dimethyl-2,8,11,12,15,16-hexahydro-3,5-etheno-10,14-methanotripyrazolo[3,4-h:3′,4′-l:4″,3″-p][1,6]oxazacycloheptadecin-13(10H)-one;
2-[(10R,19E)-18-ethoxy-22-fluoro-8-methyl-2,10,11,12,13,15-hexahydro-3,5-etheno-10,14-methanotripyrazolo[3,4-h:3′,4′-l:4″,3″-p∥1,6|oxazacycloheptadecin-16(8H)-yl|ethan-1-ol;
2-{(11S,17E)-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-12-cyclopropyl-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10R,17E)-16-ethoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(11S,17E)-12-cyclopropyl-16-ethoxy-8,11-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10R,19E)-22-fluoro-8-methyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
(2S)-2- {(10S,17E)-19-fluoro-8,10,12-trimethyl-16-[(propan-2-yl)oxy]-2, 8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(11S,17E)-6,8,11-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][,4]oxazacyclopentadecin-14-yl}ethan-1-ol;
2-{(10R,17E)-6,8,10-trimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
{[(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-cyclopropyl-16-ethoxy-6,8,10-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-cyclopropyl-16-ethoxy-6,8,11-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(10S,17E)-16-ethoxy-12-ethyl-19-fluoro-8,10,14-trimethyl-2,10,11,12,13,14-hexahydro-8H-3,5-ethenotripyrazolo[3,4-f:3′,4′-j:4″,3″-n][1,4]oxazacyclopentadecine;
(2S)-2-{(10R,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(10S,17E)-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecine-16-carbonitrile;
(10S,17E)-12-ethyl-8,10,14-trimethyl-2,10,11,12,13,14-hexahydro-8H-3,5-ethenotripyrazolo[3,4-f:3′,4′-j:4″,3″-n][1,4]oxazacyclopentadecin-16-ol;
(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-ol;
2-[(10S,17E)-19-fluoro-8,10,12-trimethyl-16-(2,2,2-trifluoroethoxy)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-{(10R,17E)-12-cyclopropyl-8,10-dimethyl-6-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(11S,17E)-12-cyclopropyl-8,11-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2S)-2-[(10S,17E)-16-ethoxy-19-fluoro-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
1-[(10R,17E)-16-ethoxy-14-(2-hydroxyethyl)-8,10-dimethyl-2,8,10,11,13,14-hexahydro-12H-3,5-ethenotripyrazolo[3,4-f:3′,4′-j:4″,3″-n][1,4]oxazacyclopentadecin-12-yl]ethan-1-one;
ethyl [(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]acetate;
(2S)-2-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(10S,17E)-14-(2-hydroxyethyl)-8,10,12-trimethyl-16-[(propan-2-yl)oxy]-2,12,13,14-tetrahydro-8H-3,5-ethenotripyrazolo[3,4-f:3′,4′-j:4″,3″-n][1,4]oxazacyclopentadecin-11(101H)-one;
2-[(10S,17E)-16-{[1-(methanesulfonyl)azetidin-3-yl]oxy}-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
1-[(10S,17E)-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]-2-methylpropan-2-ol;
2-{(10S,13R,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,13S,17E)-8,10,12,13-tetramethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-10-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(17E)-11-(fluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2S)-1-{(10R,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
(2S)-2-[(11S,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-[(10R,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(2S)-2-{(11S,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
(2S)-2-{(10R,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-1-ol;
2-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}acetanide;
(10S,17E)-19-fluoro-14-(2-hydroxyethyl)-8,10,12-trimethyl-2-(propan-2-yl)-2,10,11,12,13,14-hexahydro-8H-3,5-ethenotripyrazolo[3,4-f:3′,4′-j:4″,3″-n][1,4]oxazacyclopentadecin-16-ol;
2-[(8aR,19E)-1-(cyclopropylmethoxy)-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
(10R,18E)-17-ethoxy-8,15-dimethyl-2,8,11,12,14,15-hexahydro-10H-3,5-etheno-10,13-methanotripyrazolo[3,4-g:3′,4′-k:4″,3″-o][1,5]oxazacyclohexadecine;
{(17E)-14-(2-hydroxyethyl)-12-(propan-2-yl)-16-[(propan-2-yl)oxy]-2,10,11,12,13,14-hexahydro-7H-3,5-ethenotripyrazolo[3,4-f:3′,4′-j:4″,3″-n][1,4]oxazacyclopentadecin-7-yl}acetonitrile;
(17E)-16-ethoxy-8,14-dimethyl-2,8,10,11,13,14-hexahydro-3,5-etheno-10,12-methanotripyrazolo[3,4-f:3′,4′-j:4″,3″-n][1,4]oxazacyclopentadecine;
2-[(10S,17E)-16-ethoxy-19-fluoro-6,8,10-trimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(8aR,9R,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-1-ethoxy-22-fluoro-9,11-dimethyl-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-18-ethoxy-22-fluoro-8,21-dimethyl-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(10S,17E)-8-cyclopropyl-10,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(10S,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13R,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10R,13S,19E)-18-ethoxy-8,13,16-trimethyl-2,8,10,11,12,13,15,16-octahydro-3,5-etheno-10,14-methanotripyrazolo[3,4-h:3′,4′-l:4″,3″-p][1,6]oxazacycloheptadecine;
(10S,13R,19E)-18-ethoxy-8,13,16-trimethyl-2,8,10,11,12,13,15,16-octahydro-3,5-etheno-10,14-methanotripyrazolo[3,4-h:3′,4′-1:4″,3″-p][1,6]oxazacycloheptadecine;
2-{(10R,17E)-19-fluoro-8,10,12-trimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(8aR,9S,19E)-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9R,19E)-9,11-dimethyl-1-[(propan-2-yl)oxy]-7,8,8a,9,11,17-hexahydro-6H-14,16-ethenotripyrazolo[3,4f:3′,4′-j:4″,3″-n]pyrrolo[2,1-c][1,4]oxazacyclopentadecin-3(4H)-yl]ethan-1-ol;
2-[(19E)-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-[(8aR,9R,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(8aR,9S,19E)-22-fluoro-9,11-dimethyl-1-[(propan-2-yl)oxy]-7,8,8a,9,11,17-hexahydro-6H-14,16-ethenotripyrazolo[3,4-f:3′,4′-j:4″,3″-n]pyrrolo[2,1-c][1,4]oxazacyclopentadecin-3(4H)-yl]ethan-1-ol;
2-[(19E)-22-fluoro-8,21-dimethyl-18-[(propan-2-yl)oxy]-2,10,11,12,13,15-hexahydro-3,5-etheno-10,14-methanotripyrazolo[3,4-h:3′,4′-l:4″,3″-p][1,6]oxazacycloheptadecin-16(8H)-yl]ethan-1-ol;
2-{(10R,17E)-10-(hydroxymethyl)-8,12-dimethyl-16-|(propan-2-yl)oxy-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11S,17E)-16-ethoxy-11-ethyl-8,12-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-ethoxy-10-ethyl-8,12-dimethyl-2,8,10,11,12,13-hexahydro-14H-3,5-ethenotripyrazolo[3,4f:3′,4′-j:4″,3″-n][1,4]oxazacyclopentadecin-14-yl]ethan-1-ol;
2-[(10S,17E)-16-(methoxymethyl)-8,10,12-trimethyl-2,8,10,11,12,13-hexahydro-14H-3,5-ethenotripyrazolo[3,4f:3′,4′-j:4″,3″-n][1,4]oxazacyclopentadecin-14-yl]ethan-1-ol;
1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}-2-methylpropan-2-ol;
1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-one;
(10R,17E)-14-(2-hydroxyethyl)-8,12-dimethyl-16-[(propan-2-yl)oxy]-2,10,11,12,13,14-hexahydro-8H-3,5-ethenotripyrazolo[3,4-f:3′,4′-j:4″,3″-n][1,4]oxazacyclopentadecine-10-carbonitrile;
2-{(10S,17E)-8,10,12-trimethyl-16-[(methylamino)methyl]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-16-[(dimethylamino)methyl]-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
(2R)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
(2S)-2-{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}propanenitrile;
2-|[(4aS,7aS,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
2-[(4aR,7aR,13E)-12-ethoxy-3,6,8-trimethyl-4a,5,6,7,7a,8,9,16-octahydro-17,19-ethenotripyrazolo[3,4-f:3′,4′-j:4″,3″-n]pyrrolo[3,4-b][1,4]oxazacyclopentadecin-10(3H)-yl]ethan-1-ol;
{[(8aR,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
{[(8aR,9R,19E)-3-(2-hydroxyethyl)-9,11-dimethyl-3,4,7,8,8a,9,11,17-octahydro-6H-14,16-ethenotripyrazolo[3,4-f:3′,4′-j:4″,3″-n]pyrrolo[2,1-c][1,4]oxazacyclopentadecin-1-yl]oxy}acetonitrile;
2-{(10S,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10R,17E)-10-(difluoromethyl)-8,12-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(4aS,7aS,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(4aR,7aR,13E)-12-ethoxy-3,8,10-trimethyl-3,4a,5,6,7,7a,8,9,10,16-decahydro-17,19-ethenotripyrazolo[3,4-f:3′,4′-j:4″,3″-n]pyrrolo[3,4-b][1,4]oxazacyclopentadecine;
(2S)-1-{(10S,17E)-12-ethyl-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-{(11R,17E)-19-fluoro-8,11-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-{(10S,17E)-19-fluoro-8,10-dimethyl-12-(propan-2-yl)-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}ethan-1-ol;
2-[(11R,17E)-16-ethoxy-19-fluoro-8,11-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-|(10S,17E)-16-ethoxy-19-fluoro-8,10-dimethyl-12-(propan-2-yl)-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
{[(10S,17E)-19-fluoro-14-(2-hydroxyethyl)-8,10,12-trimethyl-2,10,11,12,13,14-hexahydro-8H-3,5-ethenotripyrazolo[3,4-f:3′,4′-j:4″,3″-n][1,4]oxazacyclopentadecin-16-yl]oxy}acetonitrile;
2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2-[(10S,17E)-16-ethoxy-12-ethyl-19-fluoro-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10R,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
(10S,17E)-16-ethoxy-12-ethyl-14-(2-hydroxyethyl)-8,10-dimethyl-2,10,11,12,13,14-hexahydro-8H-3,5-ethenotripyrazolo[3,4-f:3′,4′-j:4″,3″-n][1,4]oxazacyclopentadecine-6-carbonitrile;
2-[(10S,17E)-16-ethoxy-6-ethynyl-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
2-[(10S,17E)-16-(ethylamino)-8,10,12-trimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-1-{(10S,17E)-12-ethyl-19-fluoro-8,10-dimethyl-16-[(propan-2-yl)oxy]-2,8,10,11,12,13-hexahydro-14H-3,5-ethenotripyrazolo[3,4-f:3′,4′-j:4″,3″-n][1,4]oxazacyclopentadecin-14-yl}propan-2-ol;
2-[(10S,17E)-6-amino-16-ethoxy-12-ethyl-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]ethan-1-ol;
(2S)-2-[(10S,17E)-12-ethyl-19-fluoro-16-methoxy-8,10-dimethyl-2,8,10,11,12,13-hexahydro-14H-3,5-ethenotripyrazolo[3,4-f:3′,4′-j:4″,3″-n][1,4]oxazacyclopentadecin-14-yl]propan-1-ol;
2,2′-[(10S,17E)-10,12-dimethyl-16-[(propan-2-yl)oxy]-10,11,12,13-tetrahydro-8H-3,5-ethenotripyrazolo|3,4-f:3′,4′-j:4″,3″-n∥1,4|oxazacyclopentadecine-8,14(2H)-diyl|di(ethan-1-ol);
(17E)-16-ethyl-8,14-dimethyl-2,8,10,11,13,14-hexahydro-3,5-etheno[1,4]dioxacyclopentadecino[10,11-c:15,14-c′:6,7-c″]tripyrazole;
2-[(17E)-8,16-dimethyl-2,10,11,13-tetrahydro-3,5-etheno[1,4]dioxacyclopentadecino[10,11-c:15,14-c′:6,7-c′]tripyrazol-14(8H)-yl]-N-ethylacetamide;
(17E)-8,14-dimethyl-2,8,10,11,13,14-hexahydro-3,5-etheno[1,4]dioxacyclopentadecino[10,11-c:15,14-c′:6,7-c″]tripyrazole-16-carbonitrile;
(17E)-8,14,16-trimethyl-2,8,10,11,13,14-hexahydro-3,5-etheno[1,4]dioxacyclopentadecino[10,11-c:15,14-c′:6,7-c″]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″]tripyrazole;
(10S,17E)-8,10,14-trimethyl-2,8,10,11,13,14-hexahydro-3,5-etheno[1,4]dioxacyclopentadecino[10,11-c:15,14-c′:6,7-c″]tripyrazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]oxazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[6,5-d][1,2]thiazole-16-carbonitrile;
(10S,17E)-10,14-dimethyl-10,11,13,14-tetrahydro-2H-3,5-ethenodipyrazolo[4′,3′:13,14;4″,3″:9,10][1,4]dioxacyclopentadecino[5,6-c][1,2]oxazole-16-carbonitrile;
(10S,17E)-10, 14-dimethyl-10,11,13,14-tetrahydro-2H-3,5-ethenodipyrazolo[4′,3′:13,14;4″,3″:9,10][1,4]dioxacyclopentadecino[5,6-c][1,2]thiazole-16-carbonitrile; and
(10S,17E)-16-ethyl-8,10,14-trimethyl-2,8,10,11,13,14-hexahydro-3,5-etheno[1,4]dioxacyclopentadecino[10,11-c:15,14-c′:6,7-c″]tripyrazole;
or a pharmaceutically acceptable salt thereof.

33. A compound selected from the group consisting of (17E)-8,15,16-trimethyl-2,8,9,11,12,15-hexahydro-3,5-etheno[1,4]dioxacyclopentadecino[11,10-c:15,14-c′:6,7-c″]tripyrazole;
(17E)-8,15,16-trimethyl-2,11,12,15-tetrahydro-8H,10H-3,5-etheno[1,5]dioxacyclopentadecino[10,11-c:15,14-c′:6,7-c″]tripyrazole;
1-|(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]propan-2-one;
1-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]-2-methylpropan-2-ol;
2-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]ethan-1-ol;
(17E)-8,16-dimethyl-15-[2-(pyrrolidin-1-yl)ethyl]-2,11,12,15-tetrahydro-8H,10H-3,5-etheno[1,5]dioxacyclopentadecino[10,11-c:15,14-c′:6,7-c″]tripyrazole;
(10S,17E)-8,10,15,16-tetramethyl-2,10,11,12,13,15-hexahydro-8H-3,5-ethenotripyrazolo[3,4-f:3′,4′-j:4″,3″-n][1,4]oxazacyclopentadecine;
(17E)-8,15,16-trimethyl-2,10,11,12,13,15-hexahydro-8H-3,5-ethenotripyrazolo[3,4-f:3′,4′-j:4″,3″-n][1,4]oxazacyclopentadecine;
1-[(17E)-8,15,16-trimethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(17E)-8,12,15,16-tetramethyl-2,10,11,12,13,15-hexahydro-8H-3,5-ethenotripyrazolo[3,4-f:3′,4′-j:4″,3″-n][1,4]oxazacyclopentadecine;
1-[(10S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]ethan-1-one;
(10S,17E)-8,10,12,15,16-pentamethyl-2,10,11,12,13,15-hexahydro-8H-3,5-ethenotripyrazolo[3,4-f:3′,4′-j:4″,3″-n][1,4]oxazacyclopentadecine;
2-[(10S,17E)-8,10,16-trimethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
2-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]ethan-1-ol;
(10S,17E)-12-ethyl-8,10,15,16-tetramethyl-2,10,11,12,13,15-hexahydro-8H-3,5-ethenotripyrazolo[3,4-f:3′,4′-j:4″,3″-n][1,4]oxazacyclopentadecine;
1-[(1S,17E)-8,10,15,16-tetramethyl-2,8,10,11,13,15-hexahydro-12H-3,5-ethenotripyrazolo[3,4-f:3′,4′-j:4″,3″-n][1,4]oxazacyclopentadecin-12-yl]propan-1-one;
2-[(19E)-8,13,18-trimethyl-2,8,11,12,13,14-hexahydro-10H,17H-3,5-etheno[1,7]dioxacycloheptadecino[12,13-c:17,16-c′:8,9-c″]tripyrazol-17-yl]ethan-1-ol;
(13E)-3-methyl-3,5,6,7,8,16-hexahydro-9,12-(azeno)-17,19-ethenodipyrazolo [3,4-1:4′,3′-p][1,6]oxazacycloheptadecine;
(18E)-8-methyl-2,8,10,11,12,13-hexahydro-3,5-ethenotripyrazolo[1,5-f:3′,4′-j:4″,3″-n][1,6]oxazacyclopentadecine;
(17E)-15-(2-methoxyethyl)-8,16-dimethyl-2,11,12,15-tetrahydro-8H,10H-3,5-etheno[1,5]dioxacyclopentadecino[10,11-c:15,14-c′:6,7-c″]tripyrazole;
2-[(17E)-8,9,16-trimethyl-8,9,11,12-tetrahydro-3,5-etheno[1,4]dioxacyclopentadecino[11,10-c:15,14-c′:6,7-c′]tripyrazol-15(2H)-yl]ethan-1-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]piperidin-3-ol;
(rac)-4-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]-1-methylpiperidin-3-ol;
(17E)-8,9,16-trimethyl-15-[2-(pyrrolidin-1-yl)ethyl]-2,8,9,11,12,15-hexahydro-3,5-etheno[1,4]dioxacyclopentadecino[11,10-c:15,14-c′:6,7-c″]tripyrazole;
(rac)-1,5-anhydro-2,3-dideoxy-3-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]-L-threo-pentitol;
1,5-anhydro-2,4-dideoxy-2-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]-L-threo-pentitol;
(10S,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(10S,17E)-8,10,12,16-tetramethyl-15-[2-(4-methylpiperazin-1-yl)ethyl]-2,10,11,12,13,15-hexahydro-8H-3,5-ethenotripyrazolo[3,4-f:3′,4′-j:4″,3″-n][1,4]oxazacyclopentadecine;
(20E)-8,18-dimethyl-2,8,11,12-tetrahydro-10H-3,5-ethenodipyrazolo[3″,4″:10′,11′;4′″,3′″:14′,15′][1,5]dioxacyclopentadecino[6′,7′:3,4]pyrazolo[1, 5-a]pyrazin-19(18H)-one;
(10S,17E)-8,10,12,16-tetramethyl-15-[2-(morpholin-4-yl)ethyl]-2,10,11,12,13,15-hexahydro-8H-3,5-ethenotripyrazolo[3,4-f:3′,4′-j:4″,3″-n][1,4]oxazacyclopentadecine;
4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
N¹—{(1s,3s)-3-[(17E)-8,16-dimethyl-2,8,11,12-tetrahydro-10H,15H-3,5-etheno[1,5]dioxacyclopentadecino[10,11-c:15,14-c′:6,7-c″]tripyrazol-15-yl]cyclobutyl}-N¹,N²,N²-trimethylethane-1,2-diamine;
2-methyl-4-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]butan-2-ol;
2-methyl-1-|[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
N²- {(1s,3s)-3-[(17E)-8,16-dimethyl-2, 8,11,12-tetrahydro-10H,15H-3, 5-etheno[1,5]dioxacyclopentadecino[10,11-c:15,14-c′:6,7-c″]tripyrazol-15-yl]cyclobutyl}-N¹,N₁-dimethylethane-1,2-diamine;
(17E)-8,10,16-trimethyl-15-(1-methylpyrrolidin-3-yl)-2,9,10,11,12,15-hexahydro-8H-3,5-ethenotripyrazolo[3,4-f:4′,3′-j:4″,3″-n][1,4]oxazacyclopentadecine;
(10R,17E)-8,10,12,16-tetramethyl-15-[2-(pyrrolidin-1-yl)ethyl]-2,10,11,12,13,15-hexahydro-8H-3,5-ethenotripyrazolo[3,4-f:3′,4′-j:4″,3″-n][1,4]oxazacyclopentadecine;
(2S)-1-[(10S,17E)-8,10,12,16-tetramethyl-2, 8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,15-pentamethyl-2,10,11,12,13,15-hexahydro-8H-3,5-ethenotripyrazolo[4,3-f:3′,4′-j:4″,3″-n][1,4]oxazacyclopentadecine;
(2R)-1-[(10S,17E)-8,10,12,16-tetramethyl-2,8,10,11,12,13-hexahydro-15H-3,5-ethenotripyrazolo[3,4-f:3′,4′-j:4″,3″-n][1,4]oxazacyclopentadecin-15-yl]propan-2-ol;
(10S,17E)-8,10,12,14,16-pentamethyl-2,10,11,12,13,16-hexahydro-8H-3,5-ethenotripyrazolo[4,3-f:3′,4′-j:4″,3″-n][1,4]oxazacyclopentadecine;
(10S,17E)-8,10,12,16-tetramethyl-2,8,10,11,12,13-hexahydro-3,5-etheno[1,2]oxazolo[3,4-f]dipyrazolo[3,4-j:4′,3′-n][1,4]oxazacyclopentadecine;
(10S,17E)-8,10,12,16-tetramethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12,16-tetramethyl-2,8,10,11,12,13-hexahydro-3,5-ethenodipyrazolo [3,4-j:4′,3′-n][1,2]thiazolo[3,4-f][1,4]oxazacyclopentadecine;
(10S,20E)-18-[2-(methanesulfonyl)ethyl]-8,10,12-trimethyl-2,8,10,11,12,13,16,17,18,19-decahydro-3,5-ethenopyrazino[1′,2′:1,5]pyrazolo[3,4-f]dipyrazolo[3,4-j:4′,3′-n][1,4]oxazacyclopentadecine;
(10S,17E)-16-cyclopropyl-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethyl-8,10,12-trimethyl-2,8,10,11,12,13-hexahydro-3,5-etheno┌1,2┐oxazolo┌5,4-f┐dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-8,10,12-trimethyl-16-(propan-2-yl)-2,8,10,11,12,13-hexahydro-3,5-etheno[1,2]oxazolo[5,4-f]dipyrazolo[3,4-j:4′,3′-n][1,4]oxazacyclopentadecine;
(10S,17E)-16-ethoxy-8,10,12-trimethyl-2,8,10,11,12,13-hexahydro-3,5-etheno[1,2]oxazolo[5,4-f]dipyrazolo[3,4-j:4′,3′-n][1,4]oxazacyclopentadecine;
(10R,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,14,15-tetramethyl-12-(propan-2-yl)-2,10,11,12,13,15-hexahydro-8H-3,5-ethenotripyrazolo[4,3-f:3′,4′-j:4″,3″-n][1,4]oxazacyclopentadecine;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]oxazole;
(10S,17E)-8,10-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole-16-carbonitrile;
(10S,17E)-8,10,16-trimethyl-2,10,11,13-tetrahydro-8H-3,5-ethenodipyrazolo[3′,4′:10,11;4″,3″:14,15][1,4]dioxacyclopentadecino[7,6-d][1,2]thiazole;
(10S,17E)-8,10-dimethyl-2,10,11,13-tetrahydro-8H-3,5-ethenodipyrazolo [3′,4′:10,11;4″,3″:14,15][1,4]dioxacyclopentadecino[7,6-d][1,2]thiazole-16-carbonitrile;
(10S,17E)-8,10,16-trimethyl-2,8,10,11-tetrahydro-13H-3,5-ethenodipyrazolo[3′,4′:10, 11;4″,3″:14,15][1,4]dioxacyclopentadecino[6,7-c][1,2]oxazole;
(10S,17E)-8,10-dimethyl-2,8,10,11-tetrahydro-13H-3,5-ethenodipyrazolo [3′,4′:10,11;4″,3″:14,15][1,4]dioxacyclopentadecino[6,7-c][1,2]oxazole-16-carbonitrile;
(10S,17E)-8,10,16-trimethyl-2,8,10,11-tetrahydro-13H-3,5-ethenodipyrazolo [3′,4′:10,11;4″,3″:14,15][1,4]dioxacyclopentadecino[6,7-c][1,2]thiazole;
(10S,17E)-8,10-dimethyl-2,8,10,11-tetrahydro-13H-3,5-ethenodipyrazolo [3′,4′:10,11;4″,3″:14,15][1,4]dioxacyclopentadecino[6,7-c][1,2]oxazole-16-carbonitrile;
(10S,17E)-8,10-dimethyl-2,8,10,11-tetrahydro-13H-3,5-ethenodipyrazolo[3′,4′:10,11;4″,3″:14,15][1,4]dioxacyclopentadecino[6,7-c][1,2]thiazole-16-carbonitrile;
(17E)-8,16-dimethyl-2,18,10,11-tetrahydro-13H-3,5-ethenodipyrazolo [3′,4′:10,11;4″,3″:14,15][1,4]dioxacyclopentadecino[6,7-c][1,2]oxazole;
(17E)-8,16-dimethyl-2,10,11,13-tetrahydro-8H-3,5-ethenodipyrazolo[3′,4′:10,11;4″,3″:14,15][1,4]dioxacyclopentadecino[7,6-d][1,2]oxazole; and
(17E)-8,16-dimethyl-2, 8,10,11-tetrahydro-13H-3,5-ethenodipyrazolo [3′,4′:10,11;4″,3″:14,15][1,4]dioxacyclopentadecino[6,7-c][1,2]thiazole;
or a pharmaceutically acceptable salt thereof.

34. A pharmaceutical composition comprising a compound of any one of the preceding claims, and optionally one or more excipients.

35. A method of treating disease in a subject comprising, administering a therapeutically effective amount of a compound of any one of claims 1 to 33, or a pharmaceutical composition of claim 35.

36. A compound according to any one of claims 1 to 33, for use in a method of treating disease in a subject.

37. Use of a compound according to any one of claims 1 to 33 in the manufacture of a medicament for the treatment of disease in a subject.
